(19) United States
(12) Patent Application Publication
(10) Pub. No.: US 2026/0317356 A1
(43) Pub. Date: Oct. 15, 2026

(54) SMALL MOLECULE MODULATORS OF OGG1

(71) Applicant: AZKARRA THERAPEUTICS, INC., South San Francisco, CA (US)

(72) Inventors: Michael G. Johnson, San Francisco, CA (US); David C. Spellmeyer, Oakland, CA (US); Marc Adler, Orinda, CA (US); Dana K. Winter, Rigaud (CA); Stéphanie Roy, Lachine (CA); Guillaume Pelletier, Saint-Lazare (CA); Hong Wang, Montreal (CA); Polina Novoseltseva, Montreal (CA); Purvish Yogesh Patel, Montreal (CA); Harjeet Singh Soor, Montreal (CA); Pradeep Singh Chauhan, Montreal (CA)

(21) Appl. No.: 19/476,790

(22) PCT Filed: Apr. 18, 2024
(86) PCT No.: PCT/US2024/025135

§ 371 Date: Oct. 20, 2025

Related U.S. Application Data
(60) Provisional application No. 63/497,267, filed on Apr. 20, 2023.

(51) Int. Cl. C07F 9/6512; A61K 31/4035; A61K 31/404; A61K 31/4155; A61K 31/416; A61K 31/4178; A61K 31/4184; A61K 31/423; A61K 31/427; A61K 31/428; A61K 31/437; A61K 31/4439; A61K 31/454; A61K 31/4709; A61K 31/517; A61K 31/519; A61K 31/536; A61K 31/5377; A61K 31/675; C07D 209/46; C07D 209/50; C07D 231/56; C07D 239/80; C07D 261/20; C07D 265/18; C07D 401/04; C07D 401/12; C07D 403/04; C07D 403/06; C07D 403/12; C07D 405/04; C07D 405/12; C07D 413/04; C07D 413/12; C07D 417/12; C07D 471/04; C07D 491/107

(52) U.S. Cl. CPC C07F 9/65128; A61K 31/4035; A61K 31/404; A61K 31/4155; A61K 31/416; A61K 31/4178; A61K 31/4184; A61K 31/423; A61K 31/427; A61K 31/428; A61K 31/437; A61K 31/4439; A61K 31/454; A61K 31/4709; A61K 31/517; A61K 31/519; A61K 31/536; A61K 31/5377; A61K 31/675; C07D 209/46; C07D 209/50; C07D 231/56; C07D 239/80; C07D 261/20; C07D 265/18; C07D 401/04; C07D 401/12; C07D 403/04; C07D 403/06; C07D 403/12; C07D 405/04; C07D 405/12; C07D 413/04; C07D 413/12; C07D 417/12; C07D 471/04; C07D 491/107

(57) ABSTRACT

Small molecule modulators of OGG1, methods for the preparation of the compounds and the effect of these compounds on the enzymatic and functional activity of OGG1 are described. The methods further include treating disorders, including, cancer, cardiovascular disorders, neurological disorders, inflammatory disorders, autoimmune disorders, and infectious diseases.

### CROSS-REFERENCE TO RELATED APPLICATIONS

This application claims the benefit of U.S. Provisional Application Ser. No. 63/497,267, filed on Apr. 20, 2023, and entitled “Small Molecule Modulators of OGG1,” the entire contents of which are incorporated herein by reference.

### TECHNICAL FIELD

The disclosure relates to the preparation of small molecule compounds that inhibit the DNA repair enzyme 8-oxoguanine glycosylase 1 (OGG1) and the therapeutic agents that may be made from these compounds, and uses thereof.

### BACKGROUND

In cells, Reactive Oxygen Species (ROS) damage genomic DNA, particularly by the oxidation of individual nucleotide bases. The guanosine base is especially prone to oxidation, forming the 8-oxo-guanasine (8-oxo-G) or 2,6-diamino-4-hydroxy-5-formamidopyrimidine (FapyGua) mutagenic lesions. Left unrepaired, 8-oxo-G bases are mis-read by DNA polymerases and adenine is introduced as the complementary base instead of cytosine. Thus, after DNA replication, an 8-oxo-G lesion results in the G: C to T: A base transversion. Accumulated transversions result in replication stress ultimately triggering cell-cycle arrest, and in the most extreme cases, cell death. In order to avoid the mutagenic effect of oxidized bases in DNA, cells have specialized enzymes to recognize and remove the offending bases via the DNA Damage and Response (DDR) pathway of Base Excision Repair (BER). The enzyme 8-oxoguanine glycosylase-1 (OGG1) is responsible for recognition of the 8-oxo-G lesion in DNA, and it begins the process of removing 8-oxo-G by cleaving the glycosidic bond. Particularly in cells with high ROS, OGG1 is essential for removing 8-oxo-G lesions to maintain the integrity of genomic DNA. Aside from its enzymatic role in BER, OGG1 also acts as a transcriptional activator of pro-inflammatory genes through its association with oxidized sites in DNA.

Cancer cells with high levels of ROS and/or faulty mismatch repair mechanisms (MMR) are especially dependent on proficient OGG1 to avoid the accumulation of 8-oxo-G lesions in cellular and mitochondrial DNA. Inhibiting the enzymatic activity of OGG1 in these cells lead to the replication stress, cell cycle arrest, and apoptosis. Small molecule inhibitors of OGG1 catalytic activity are expected to directly kill cancer cells with high ROS and deficient MMR and to sensitize cancer cells to chemotherapeutics that increase ROS or otherwise increase the level of 8-oxo-G lesions in genomic DNA. Thus, it is the case that OGG1 inhibitor compounds may be used as a single agent therapeutic for cancers that are dependent on OGG1, and they may be used as a combination therapeutic for cancers that are sensitized to OGG1 inhibition through the act of a second therapeutic agent. Blocking the enzymatic activity of OGG1 can prevent the transcriptional activation of pro-inflammatory genes, and thus, OGG1 inhibitor compounds may be suitable as therapeutics for inflammatory and autoimmune disorders.

There remains a need for compounds and therapeutic agents that can block the enzymatic activity of OGG1 to prevent the transcriptional activation of pro-inflammatory genes, for the treatment of OGG1 mediated disorders, including, inflammatory and autoimmune disorders.

### SUMMARY

The present disclosure provides a compound of Formula (I):

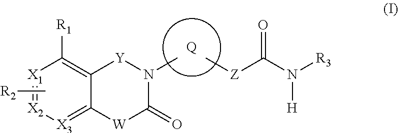

and stereoisomers, or pharmaceutically acceptable salts thereof, wherein the constituent members are defined herein throughout the disclosure.

The present disclosure further provides a pharmaceutical composition comprising a compound of the present disclosure, and stereoisomers and pharmaceutically acceptable salts thereof, and a pharmaceutically acceptable carrier.

The present disclosure further provides a compound of the present disclosure, and stereoisomers and pharmaceutically acceptable salts thereof, for use in the treatment of a disorder that is responsive to inhibition of OGG1.

The present disclosure further provides a compound of the present disclosure, and stereoisomers and pharmaceutically acceptable salts thereof, for use in the manufacture of a medicament for the treatment of a disorder that is responsive to inhibition of OGG1.

The present disclosure further provides a method of treating a disorder in a subject in need thereof, wherein the disorder is mediated by OGG1, comprising administering to the subject a compound or pharmaceutical composition of the present disclosure.

### DETAILED DESCRIPTION

The following description sets forth numerous exemplary configurations, methods, parameters, and the like. It should be recognized, however, that such description is not intended as a limitation on the scope of the present disclosure but is instead provided as a description of exemplary embodiments.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

“Alkyl”, as used herein, refers to an unbranched or branched saturated hydrocarbon chain. Alkyl can be used alone, or as part of another radical, such as —O-alkyl. In some embodiments, alkyl as used herein has 1 to 20 carbon atoms ((C₁₋₂₀)alkyl), 1 to 12 carbon atoms ((C₁₋₁₂)alkyl), 1 to 10 carbon atoms ((C₁₋₁₀)alkyl), 1 to 8 carbon atoms ((C₁₋₈)alkyl), 1 to 6 carbon atoms ((C₁₋₆)alkyl), 1 to 4 carbon atoms ((C₁₋₄)alkyl), or 1 to 3 carbon atoms ((C₁₋₃)alkyl). Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methyl pentyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

“Alkenyl”, as used herein, refers to an unbranched or branched hydrocarbon chain. The “alkenyl” group contains at least one double bond. The double bond of an alkenyl group can be unconjugated or conjugated to another group. The alkenyl may be branched or straight. In some embodiments, alkenyl as used herein has 2 to 20 carbon atoms ((C₂₋₂₀)alkenyl), 2 to 12 carbon atoms ((C₂₋₁₂)alkenyl), 2 to 10 carbon atoms ((C₂₋₁₀)alkenyl), 2 to 8 carbon atoms ((C₂₋₈)alkenyl), 2 to 6 carbon atoms ((C₂₋₆)alkenyl, 2 to 4 carbon atoms ((C₂₋₄)alkenyl), or 2 to 3 carbon atoms ((C₂₋₃)alkenyl). Examples of alkenyl groups include, but are not limited to, ethylenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl and the like. When an alkenyl residue having a specific number of carbons is named, all geometric isomers and all E-Z isomers having that number of carbons may be encompassed.

“Alkynyl”, as used herein, refers to an unbranched or branched unsaturated hydrocarbon chain. The “alkynyl” group contains at least one triple bond. The alkynyl may be branched or straight. The triple bond of an alkynyl group can be unconjugated or conjugated to another group. In some embodiments, alkynyl as used herein has 2 to 50 carbon atoms ((C₂₋₅₀)alkynyl), 2 to 20 carbon atoms ((C₂₋₂₀)alkynyl), 2 to 12 carbon atoms ((C₂₋₁₂)alkynyl), 2 to 10 carbon atoms ((C₂₋₁₀)alkynyl), 2 to 8 carbon atoms ((C₂₋₈)alkynyl), 2 to 6 carbon atoms ((C₂₋₆)alkynyl, 2 to 4 carbon atoms ((C₂₋₄)alkynyl), or 2 to 3 carbon atoms ((C₂₋₃)alkynyl). Examples of alkynyl groups include, but are not limited to, ethynyl, propynyl, butynyl, pentynyl, hexynyl, methylpropynyl, 4-methyl-1-butynyl, 4-propyl-2-pentynyl, 4-butyl-2-hexynyl and the like. When an alkynyl residue having a specific number of carbons is named, all geometric isomers having that number of carbons may be encompassed.

“Cycloalkyl”, as used herein, refers to a saturated or partially saturated, monocyclic, fused or spiro polycyclic, carbocycle having from 3 to 18 carbon atoms per ring. The cycloalkyl ring or carbocycle may be unsubstituted or substituted by one or more substituents, e.g., 1 to 5 substituents, at any point of attachment. The substituents can themselves be unsubstituted or substituted. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, and derivatives thereof.

“Heterocycle”, “heterocyclic”, “heterocyclyl”, or “heterocyclediyl”, as used herein, refers to a saturated or partially unsaturated and non-aromatic monocyclic or fused polycyclic or spiro polycyclic ring structure of 4-to-18 atoms containing carbon and heteroatoms taken from oxygen, nitrogen, or sulfur wherein there is not delocalized π-electrons (aromaticity) shared among all ring carbons or heteroatoms. A heterocyclyl ring structure attaches to a single point of a moiety of the formulae described herein, while a heterocyclediyl ring structure attaches to two points of a moiety or moieties of formulae described herein. The heterocycle, heterocyclyl, or heterocyclediyl ring structure may be unsubstituted or substituted by one or more substituents. The substituents can themselves be unsubstituted or substituted. Examples of heterocycle, heterocyclyl, or heterocyclediyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, homotropanyl, dihydrothiophen-2(3H)-onyl, tetrahydrothiophene 1,1-dioxide, 2,5-dihydro-1H-pyrrolyl, imidazolidin-2-one, pyrrolidin-2-one, dihydrofuran-2(3H)-one, 1,3-dioxolan-2-one, isothiazolidine 1,1-dioxide, 4,5-dihydro-1H-imidazolyl, 4,5-dihydrooxazolyl, oxiranyl, pyrazolidinyl, 4H-1,4-thiazinyl, thiomorpholinyl, 1,2,3,4-tetrahydropyridinyl, 1,2,3,4-tetrahydropyrazinyl, 1,3-oxazinan-2-one, tetrahydro-2H-thiopyran 1,1-dioxide, 7-oxabicyclo[2.2.1]heptanyl, 1,2-thiazepane 1,1-dioxide, octahydro-2H-quinolizinyl, 1,3-diazabicyclo[2.2.2]octanyl, 2,3-dihydrobenzo[b][1,4]dioxine, 3-azabicyclo[3.2.1]octanyl, 8-azaspiro[4.5]decane, 8-oxa-3-azabicyclo[3.2.1]octanyl, 2-azabicyclo[2.2.1]heptane, 2,8-diazaspiro[5.5]undecanyl, 2-azaspiro[5.5]undecanyl, 3-azaspiro[5.5]undecanyl, decahydroisoquinolinyl, 1-oxa-8-azaspiro[4.5]decanyl, 8-azabicyclo[3.2.1]octanyl, 1,4′-bipiperidinyl, azepanyl, 8-oxa-3-azabicyclo[3.2.1]octanyl, 3,4-dihydro-2H-benzo[b][1,4]oxazinyl, 5,6,7,8-tetrahydroimidazo[1,2-a]pyridinyl, 1,4-diazepanyl, phenoxathiinyl, benzo[d][1,3]dioxolyl, 2,3-dihydrobenzofuranyl, 2,3-dihydrobenzo[b][1,4] dioxinyl, 4-(piperidin-4-yl)morpholinyl, 3-azaspiro[5.5]undecanyl, decahydroquinolinyl, piperazin-2-one, 1-(pyrrolidin-2-ylmethyl)pyrrolidinyl, 1,3′-bipyrrolidinyl, and 6,7,8,9-tetrahydro-1H,5H-pyrazolo[1,2-a][1,2]diazepinyl.

“Aryl”, as used herein, refers to a monocyclic or polycyclic group having at least one hydrocarbon aromatic ring wherein all of the ring atoms of the at least one hydrocarbon aromatic ring are carbon. Aryl may include groups with a single aromatic ring (e.g., phenyl) and multiple fused aromatic rings (e.g., naphthyl, anthryl). Aryl may further include groups with one or more aromatic hydrocarbon rings fused to one or more non-aromatic hydrocarbon rings (e.g., fluorenyl; 2,3-dihydro-1H-indene; 1,2,3,4-tetrahydronaphthalene). In certain embodiments, aryl includes groups with an aromatic hydrocarbon ring fused to a non-aromatic ring wherein the non-aromatic ring comprises at least one ring hetero atom independently selected from the group consisting of N, O, and S. For example, in some embodiments, aryl includes groups with a phenyl ring fused to a non-aromatic ring, wherein the non-aromatic ring comprises at least one ring hetero atom independently selected from the group consisting of N, O, and S (e.g., chromane; thiochromane; 2,3-dihydrobenzofuran; indoline). In some embodiments, aryl as used herein has from 6 to 14 carbon atoms ((C₆-C₁₄)aryl), or 6 to 10 carbon atoms ((C₆-C₁₀)aryl). Where the aryl includes fused rings, the aryl may connect to one or more substituents or moieties of the formulae described herein through any atom of the fused ring for which valency permits.

“Heteroaryl”, as used herein, refers to a monocyclic or polycyclic group comprising at least one aromatic ring, wherein the aromatic ring comprises at least one ring heteroatom independently selected from the group consisting of N, O, and S. The heteroaryl group may comprise 5, 6, 7, 8, 9, 10, 11, 12, or more ring atoms, where ring atoms refer to the sum of carbon and heteroatoms in the one or more rings (e.g., be a 5-membered, 6-membered, 7-membered, 8-membered, 9-membered, 10-membered, 11-membered, or 12-membered heteroaryl). In some embodiments, heteroaryl includes groups with an aromatic ring that comprises at least one ring heteroatom independently selected from the group consisting of N, O, and S, (e.g., pyridinyl, pyrazinyl, furanyl, thiophenyl). In certain embodiments, heteroaryl includes polycyclic groups with an aromatic ring comprising at least one ring heteroatom, fused to a non-aromatic hydrocarbon ring (e.g., 5,6,7,8-tetrahydroquinolinyl; 4,5,6,7-tetrahydroisobenzofuranyl). In some embodiments, heteroaryl includes polycyclic groups with an aromatic ring comprising at least one ring heteroatom fused to an aromatic hydrocarbon ring (e.g., quinolinyl, quinoxalinyl, benzothiazolyl). In still further embodiments, heteroaryl includes polycyclic groups with two fused aromatic rings, wherein each ring comprises at least one ring heteroatom (e.g., naphthyridinyl). Heteroaryl may include groups comprising 1 to 5 ring heteroatoms, 1 to 4 ring heteroatoms, 1 to 3 ring heteroatoms, 1 or 2 ring heteroatoms, or 1 ring heteroatom, wherein each ring heteroatom is independently selected from the group consisting of N, O, and S. In one example, a heteroaryl has 3 to 8 ring carbon atoms, with 1 to 3 ring heteroatoms independently selected from N, O, and S. Examples of heteroaryl groups include, without limitations, pyridyl, pyridazinyl, pyrimidinyl, benzothiazolyl, and pyrazolyl.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

“Amino”, as used herein, means a substituent containing at least one nitrogen atom. For example, NH₂, —NH(alkyl) or alkylamino, —N(alkyl)₂ or dialkylamino, amide, carboxamide, urea, and sulfamide are included in the term “amino”.

“Cyano”, as used herein, refers to a substituent having a carbon atom joined to a nitrogen atom by a triple bond, i.e.,

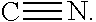

“Hydroxyl” or “hydroxy”, as used herein, refers to an OH group.

“Halogen” or “halo”, as used herein, refers to fluoro, chloro, bromo, or iodo radicals.

“Haloalkyl,” as used herein, refers to an alkyl group substituted with one or more halogen.

“Halocycloalkyl”, as used herein, refers to a cycloalkyl group substituted with one or more halogen.

“Haloaryl”, as used herein, refers to an aryl group substituted with one or more halogen.

“Oxo”, as used herein, refers to an “═O” group.

It should be understood that when a range of values is listed, it is intended to encompass each value and sub-range within the range. For example, “C₁₋₆alkyl” (which may also be referred to as C₁-C₆ alkyl, C₁-C₆ alkyl, or C₁₋₆ alkyl) is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₅, and C₅₋₆ alkyl.

It should also be noted that any carbon as well as heteroatom with unsatisfied valences in the text, schemes, examples and tables herein is assumed to have the sufficient number of hydrogen atom(s) to satisfy the valences.

As used herein, references to hydrogen may also refer to a deuterium substitution if desired. The term “deuterium” as used herein means a stable isotope of hydrogen having odd numbers of protons and neutrons.

Compounds of the various Formulae and stereoisomers and pharmaceutically acceptable salts thereof may exist in their tautomeric form (for example, as an amide or imino ether). All such tautomeric forms are contemplated herein as part of the present disclosure.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans-configuration.

The compounds of the various Formulae may contain asymmetric or chiral centers, and, therefore, exist in different stereoisomeric forms. It is intended that all stereoisomeric forms of the compounds of the various Formulae as well as mixtures thereof, including racemic mixtures, form part of the present disclosure. In some embodiments, isomers of the compounds herein are stereoisomers. In addition, the present disclosure embraces all geometric and positional isomers. For example, if a compound of the various Formulae incorporates a double bond or a fused ring, both the cis- and trans-forms, as well as mixtures, are embraced within the scope of the present disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers, and converting (e.g., hydrolyzing) the individual diastereomers to the corresponding pure enantiomers. Also, some of the compounds of the various Formulae may be atropisomers (e.g., substituted biaryls) and are considered as part of the present disclosure. Enantiomers can also be separated by use of a chiral HPLC column.

In some embodiments, the compounds of Formulae I, II, IIIa, IIIb, IVa, IVb, and pharmaceutically acceptable salts thereof are enantiomers. In some embodiments, the compounds and pharmaceutically acceptable salts thereof are the (S)-enantiomer. In other embodiments the compounds and pharmaceutically acceptable salts thereof are the (R)-enantiomer. In some embodiments, the compounds and pharmaceutically acceptable salts thereof are the (+) enantiomer or (−) enantiomer.

Some embodiments are directed to isotopically-labelled compounds of the present disclosure which are identical to those recited herein but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, fluorine and chlorine, such as ²H (or D), ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of the various Formulae (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the various Formulae can generally be prepared by following procedures analogous to those disclosed in the Schemes and/or in the Examples herein below by substituting an appropriate isotopically labelled reagent for a non-isotopically labelled reagent.

In some embodiments, the compound comprises at least one deuterium atom. For example, one or more hydrogen atoms in a compound of the present disclosure can be replaced or substituted by deuterium. In some embodiments, the compound comprises two or more deuterium atoms. In some embodiments, the compound comprises 1, 2, 3, 4, 5, 6, 7, 8, 9, 10, 11 or 12 deuterium atoms.

The compounds of Formulae I, II, IIIa, IIIb, IVa, and IVb may form salts which are also within the scope of the present disclosure. Reference to a compound of the Formula herein is understood to include reference to salts thereof, unless otherwise indicated.

The present disclosure is directed to compounds as described herein and stereoisomers and pharmaceutically acceptable salts thereof. The present disclosure is also directed to pharmaceutical compositions comprising one or more compounds as described herein and stereoisomers and pharmaceutically acceptable salts thereof.

“Pharmaceutically acceptable”, as used herein, refers to that which is useful in preparing a pharmaceutical composition that is generally safe, non-toxic and not biologically or otherwise undesirable, and includes that which is acceptable for veterinary use as well as human pharmaceutical use. For example, provided herein is a pharmaceutical composition comprising a compound of Formulae I, II, IIIa, IIIb, IVa, or IVb and stereoisomers and pharmaceutically acceptable salts thereof, and a pharmaceutically acceptable excipient.

“Pharmaceutically acceptable salt”, as used herein, refers to a salt which is generally safe, non-toxic and not biologically or otherwise undesirable, and includes that which is acceptable for veterinary use as well as human pharmaceutical use. Such salts may include acid addition salts and base addition salts. Acid addition salts may be formed with inorganic acid such as, but not limited to, hydrochloric acid, hydrobromic acid, sulfuric acid, nitric acid, phosphoric acid and the like; or an organic acid such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, or undecylenic acid. Salts derived from inorganic bases may include, but are not limited to, sodium, potassium, lithium, ammonium, calcium, magnesium, iron, zinc, copper, manganese, and aluminum salts. Salts derived from organic bases may include, but are not limited to, salts of primary, secondary, or tertiary amines; substituted amines including naturally occurring substituted amines; cyclic amines; ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, or N-ethylpiperidine.

The term “carrier”, as used herein, encompasses carriers, excipients, and diluents and refers to a material, composition, or vehicle such as a liquid or solid filler, diluent, excipient, solvent, or encapsulating material involved in carrying or transporting a pharmaceutical agent from one organ, or portion of the body, to another organ, or portion of the body, of a subject. Excipients should be selected on the basis of compatibility and the release profile properties of the desired dosage form. Exemplary carrier materials include, e.g., binders, suspending agents, disintegration agents, filling agents, surfactants, solubilizers, stabilizers, lubricants, wetting agents, diluents, spray-dried dispersions, and the like.

“Pharmaceutically compatible carrier materials” may include, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Hoover, John E., Remington's Pharmaceutical Sciences, Mack Publishing Co., Easton, Pa. 1975.

“Solvate”, as used herein, refers to a complex of variable stoichiometry formed by a solute and solvent. Such solvents for the purpose of the present disclosure may not interfere with the biological activity of the solute. Examples of suitable solvents include, but are not limited to, water, methanol, ethanol, and acetic acid. Solvates wherein water is the solvent molecule are typically referred to as hydrates. Hydrates include compositions containing stoichiometric amounts of water, as well as compositions containing variable amounts of water.

### Compounds

The present disclosure provides a compound of Formula I:

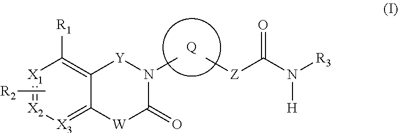

or a stereoisomer, or a pharmaceutically acceptable salt thereof, wherein:
X₁, X₂ and X₃ are each independently CH, C, or N;
R₁ is R, halo, tert-butyl, cyclopropyl, cyclobutyl, CF₃, CHF₂, CH₂F, OH, CN, CHO, C(O)Me, C(O)OMe, C(O)NH₂, CHCH₂, NR′R″, R′NR″R′″;
R₂ is absent, C₁-C₃ alkyl, C₁-C₃ alkene, NR′R″, NHR′NR″R″, R′NR″R″, NHC(O)R′NR″R″, OR, CN, NO₂, C(O)R, R′OR″, R′NR″C(O)R″, C(O)OR, C(O)NR′R″, C(O)NHR′NR″R″, P(O)R′R″, R′NR″S(O)₂R″, —O—R′NR″R″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹;
Y is CR′R″, C(O), O, S, or NR;
W is CH₂, O, S, NR or a single bond;
Q is A², optionally comprising one or more of O, N, or NR;
Z is CH₂, S, O, NR or a single bond;
R₃ is A³, B², or a fused bicyclic aromatic ring system, wherein the fused bicyclic aromatic ring system optionally comprises one or more of N, NR, O, or S;
A¹, A², and A³ are each independently a 3- to 7-membered carbocyclic or heterocyclic ring;
B¹ and B² are each independently a 5- or 6-membered aromatic or heteroaromatic ring;
wherein R, R′, R″, and R′″ are each independently H, C₁₋₆ alkyl, or C₁₋₆ alkene;
wherein the R, R′, R″, R″, A¹, A², A³, B¹, B², and the fused bicyclic aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

In some embodiments, the compound is of Formula (II):

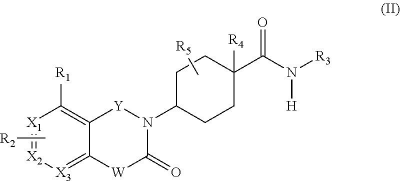

or a stereoisomer, or a pharmaceutically acceptable salt thereof, wherein:
X₁, X₂ and X₃ are independently CH, C, or N;
R₁ is R, halo, tert-butyl, cyclopropyl, cyclobutyl, CF₃, CHF₂, CH₂F, OH, CN, CHO, C(O)Me, C(O)OMe, C(O)NH₂, CHCH₂, NR′R″, R′NR″R″;
R₂ is absent, C₁-C₃ alkyl, C₁-C₃ alkene, NR′R″, NHR′NR″R″, R′NR″R″, NHC(O)R′NR″R″, OR, CN, NO₂, C(O)R, R′OR″, R′NR″C(O)R″, C(O)OR, C(O)NR′R″, C(O)NHR′NR″R″, P(O)R′R″, R′NR″S(O)₂R″, —O—R′NR″R″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹;
Y is CR′R″, C(O), O, S, or NR;
W is CH₂, O, S, NR or a single bond;
R⁵ is H₂, C₁₋₆ alkyl, C₁₋₆ alkene, OR, NR′R″, halo, or CN;
R⁴ is H, C₁₋₆ alkyl, OR, NR′R″, halo, CN;
R₃ is A³, B², or a fused bicyclic aromatic ring system, wherein the fused bicyclic aromatic ring system optionally comprises one or more of N, NR, O, or S;
A¹ and A³ are each independently a 3- to 7-membered carbocyclic or heterocyclic ring; and
B¹ and B² are each independently a 5- or 6-membered aromatic or heteroaromatic ring;
wherein R₄ and/or R₅ are optional;
wherein R, R′, R″, and R′″ are each independently H, C₁₋₆ alkyl, or C₁₋₆ alkene; and
wherein the R, R′, R″, R′″, A¹, A³, B¹, B², and the fused aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

In some embodiments, the compound is of Formula (IIIa) or (IIIb):

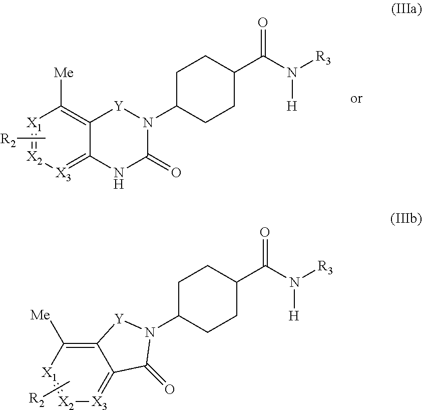

or a stereoisomer, or a pharmaceutically acceptable salt thereof, wherein:
X₁, X₂ and X₃ are independently CH, C, or N;
R₂ is absent, C₁-C₃ alkyl, C₁-C₃ alkene, NR′R″, NHR′NR″R″, R′NR″R″, NHC(O)R′NR″R″, OR, CN, NO₂, C(O)R, R′OR″, R′NR″C(O)R″, C(O)OR, C(O)NR′R″, C(O)NHR′NR″R″, P(O)R′R″, R′NR″S(O)₂R″, —O—R′NR″R″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹;
Y is CR′R″, C(O), O, S, or NR;
R₃ is A³, B², or a fused bicyclic aromatic ring system, wherein the fused bicyclic aromatic ring system optionally comprises one or more of N, NR, O, or S;
A¹ and A³ are each independently a 3- to 7-membered carbocyclic or heterocyclic ring; and
B¹ and B² are each independently a 5- or 6-membered aromatic or heteroaromatic ring;
wherein R, R′, R″, and R′″ are each independently H, C₁₋₆ alkyl, or C₁₋₆ alkene; and
wherein the R, R′, R″, R″, A¹, A³, B¹, B², and the fused bicyclic aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

In some embodiments, the compound of Formula (IIIa) has the following formula:

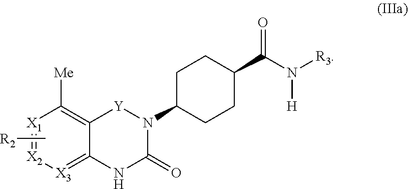

In other embodiments, the compound of Formula (IIIb) has the following formula:

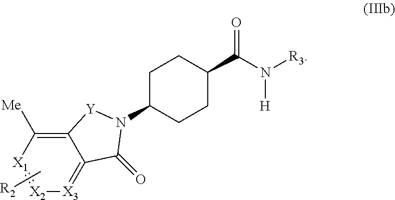

In some embodiments, the compound is of Formula (IVa) or (IVb):

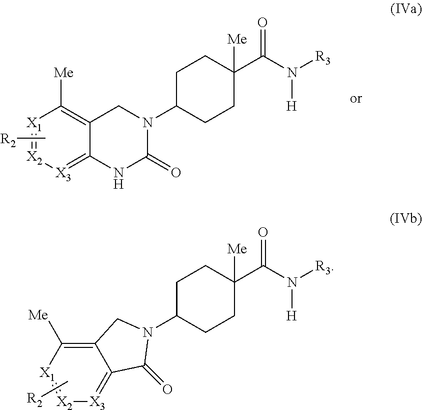

wherein:
X₁, X₂ and X₃ are independently CH, C, or N
R₂ is absent, C₁-C₃ alkyl, C₁-C₃ alkene, NR′R″, NHR′NR″R″, R′NR″R″, NHC(O)R′NR″R″, OR, CN, NO₂, C(O)R, R′OR″, R′NR″C(O)R″, C(O)OR, C(O)NR′R″, C(O)NHR′NR″R″, P(O)R′R″, R′NR″S(O)₂R″, —O—R′NR″R″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹;
R₃ is A³, B², or a fused bicyclic aromatic ring system, wherein the fused bicyclic aromatic ring system optionally comprises one or more of N, NR, O, or S;
A¹ and A³ are each independently a 3- to 7-membered carbocyclic or heterocyclic ring; and
B¹ and B² are each independently a 5- or 6-membered aromatic or heteroaromatic ring;
wherein R, R′, R″, and R′″ are each independently H, C₁₋₆ alkyl, or C₁₋₆ alkene; and
wherein the R, R′, R″, R″, A¹, A³, B¹, B², and the fused bicyclic aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

In some embodiments, the compound of Formula (IVa) has the following formula:

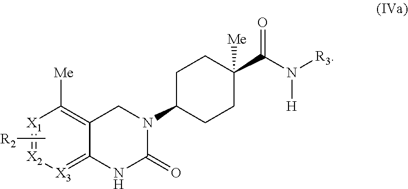

In other embodiments, the compound of Formula (IVb) has the following formula:

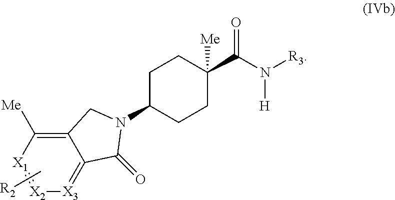

In some embodiments of the compounds, stereoisomers, or pharmaceutically acceptable salts thereof described herein, at least two of X₁, X₂, and X₃ are CH or N.

In some embodiments of the compounds, stereoisomers, or pharmaceutically acceptable salts thereof described herein, R₁ is H, C₁₋₆ alkyl, or halo.

In some embodiments of the compounds, stereoisomers, or pharmaceutically acceptable salts thereof described herein, R₂ is absent, NR′R″, OR, CN, a 5- or 6-membered heteroaromatic ring.

In some embodiments of the compounds, stereoisomers, or pharmaceutically acceptable salts thereof described herein, R₂ is attached to X₂ or X₃.

In some embodiments of the compounds, stereoisomers, or pharmaceutically acceptable salts thereof described herein, the A¹ or B¹ of R₂ is

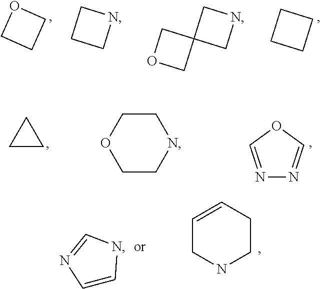

optionally substituted with one or two substituents selected from the group consisting of C₁₋₆ alkyl, —O—C₁₋₆ alkyl, CN, OH, halo, and NH₂.

In some embodiments of the compounds, stereoisomers, or pharmaceutically acceptable salts thereof described herein, Y is CR′R″ or CH₂.

In some embodiments of the compounds, stereoisomers, or pharmaceutically acceptable salts thereof described herein, in Formula (IIIa) the Y is NR, or wherein in Formula (IIIb) the Y is NR, O, or S.

In some embodiments of the compounds, stereoisomers, or pharmaceutically acceptable salts thereof described herein, Q is

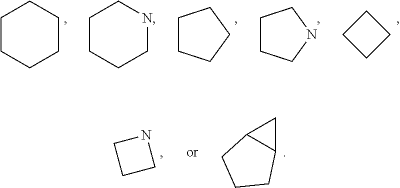

In some embodiments of the compounds, stereoisomers, or pharmaceutically acceptable salts thereof described herein, R₄ is a C₁₋₃ alkyl.

In some embodiments of the compounds, stereoisomers, or pharmaceutically acceptable salts thereof described herein, Z is a single bond.

In some embodiments of the compounds, stereoisomers, or pharmaceutically acceptable salts thereof described herein, the 3- to 7-membered carbocyclic or heterocyclic ring comprises a spirocyclic ring.

In some embodiments of the compounds, stereoisomers, or pharmaceutically acceptable salts thereof described herein, the A³ or B² of R₃ is substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

In some embodiments of the compounds, stereoisomers, or pharmaceutically acceptable salts thereof described herein, Rs is the 5- or 6-membered aromatic or heteroaromatic ring or fused bicyclic aromatic ring system substituted with one or two substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

In some embodiments of the compounds, stereoisomers, or pharmaceutically acceptable salts thereof described herein, R₃ is

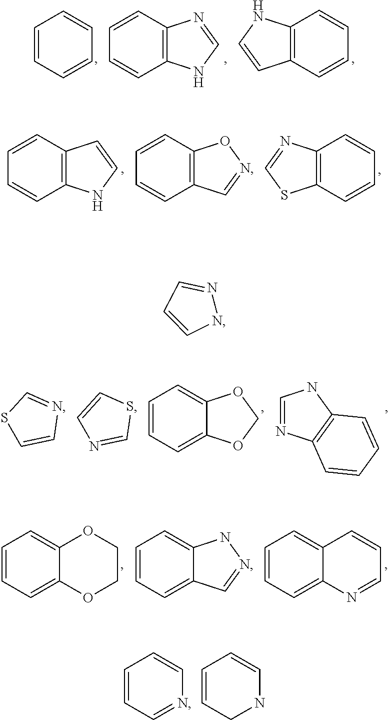

and is unsubstituted or substituted with one, two, or three substituents selected from the group consisting of CN, CF₃, OR, ═O, C₁₋₆ alkyl, —O—C₁₋₆ alkyl, C₁₋₆ alkene, ROH, halo, and a 3- to 7-membered carbocyclic or heterocyclic ring.

In some embodiments of the compounds, stereoisomers, or pharmaceutically acceptable salts thereof described herein, A² is substituted with one or two substituents selected form the group consisting of CN, CF₃, OR, ═O, ROH, NR′R″, C₁₋₆ alkyl, —O—C₁₋₆ alkyl, C₁₋₆ alkene, F, Cl, Br, I, and a 3- to 7-membered carbocyclic or heterocyclic ring.

In some embodiments of the compounds, stereoisomers, or pharmaceutically acceptable salts thereof described herein, the compound is selected from the group consisting of

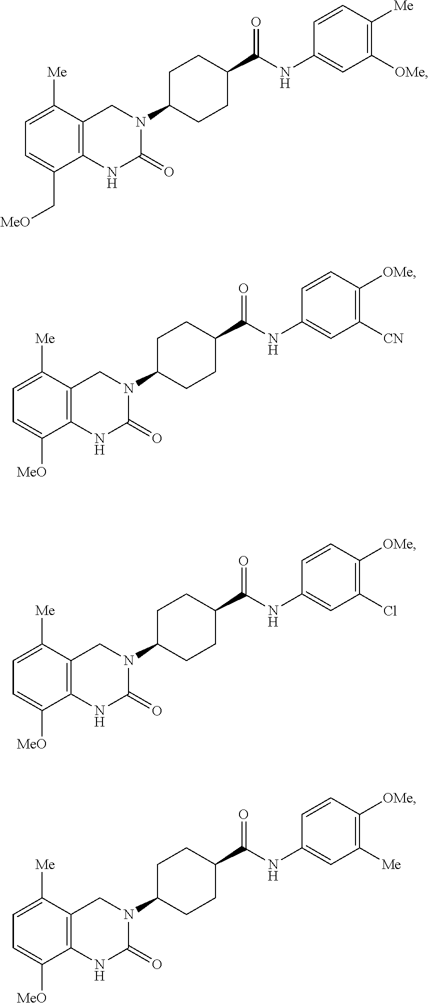 [SMILES: COCc1ccc(C)c2c1NC(=O)N([C@H]1CC[C@@H](C(=O)Nc3ccc(C)c(OC)c3)CC1)C2 ; COc1ccc(NC(=O)[C@H]2CC[C@@H](N3Cc4c(C)ccc(OC)c4NC3=O)CC2)cc1C#N ; COc1ccc(NC(=O)[C@H]2CC[C@@H](N3Cc4c(C)ccc(OC)c4NC3=O)CC2)cc1Cl ; COc1ccc(NC(=O)[C@H]2CC[C@@H](N3Cc4c(C)ccc(OC)c4NC3=O)CC2)cc1C]

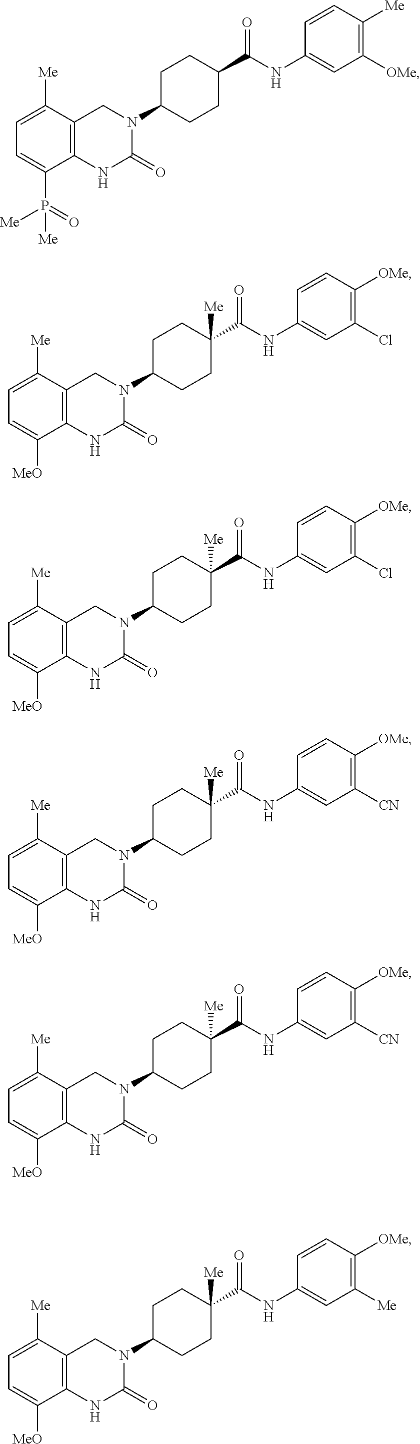 [SMILES: COc1cc(NC(=O)[C@H]2CC[C@@H](N3Cc4c(C)ccc(P(C)(C)=O)c4NC3=O)CC2)ccc1C ; COc1ccc(NC(=O)[C@]2(C)CC[C@@H](N3Cc4c(C)ccc(OC)c4NC3=O)CC2)cc1Cl ; COc1ccc(NC(=O)[C@]2(C)CC[C@H](N3Cc4c(C)ccc(OC)c4NC3=O)CC2)cc1Cl ; COc1ccc(NC(=O)[C@]2(C)CC[C@@H](N3Cc4c(C)ccc(OC)c4NC3=O)CC2)cc1C#N ; COc1ccc(NC(=O)[C@]2(C)CC[C@H](N3Cc4c(C)ccc(OC)c4NC3=O)CC2)cc1C#N ; COc1ccc(NC(=O)[C@]2(C)CC[C@@H](N3Cc4c(C)ccc(OC)c4NC3=O)CC2)cc1C]

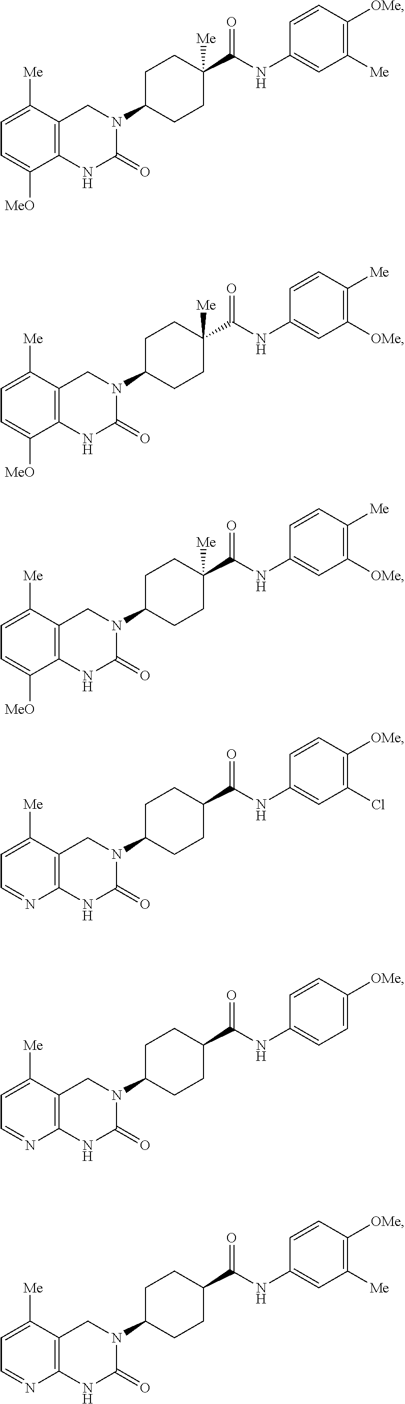 [SMILES: COc1ccc(NC(=O)[C@]2(C)CC[C@H](N3Cc4c(C)ccc(OC)c4NC3=O)CC2)cc1C ; COc1cc(NC(=O)[C@]2(C)CC[C@@H](N3Cc4c(C)ccc(OC)c4NC3=O)CC2)ccc1C ; COc1cc(NC(=O)[C@]2(C)CC[C@H](N3Cc4c(C)ccc(OC)c4NC3=O)CC2)ccc1C ; COc1ccc(NC(=O)[C@H]2CC[C@@H](N3Cc4c(C)ccnc4NC3=O)CC2)cc1Cl ; COc1ccc(NC(=O)[C@H]2CC[C@@H](N3Cc4c(C)ccnc4NC3=O)CC2)cc1 ; COc1ccc(NC(=O)[C@H]2CC[C@@H](N3Cc4c(C)ccnc4NC3=O)CC2)cc1C]

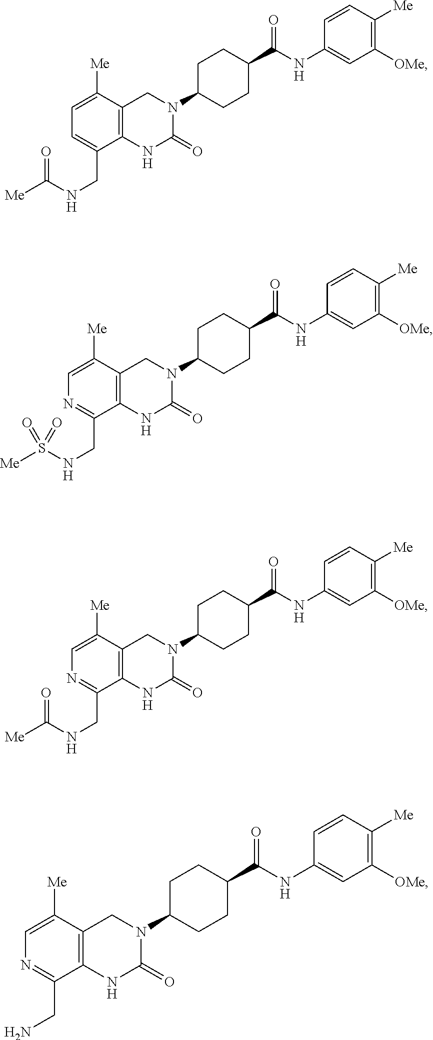 [SMILES: COc1cc(NC(=O)[C@H]2CC[C@@H](N3Cc4c(C)ccc(CNC(C)=O)c4NC3=O)CC2)ccc1C ; COc1cc(NC(=O)[C@H]2CC[C@@H](N3Cc4c(C)cnc(CNS(C)(=O)=O)c4NC3=O)CC2)ccc1C ; COc1cc(NC(=O)[C@H]2CC[C@@H](N3Cc4c(C)cnc(CNC(C)=O)c4NC3=O)CC2)ccc1C ; COc1cc(NC(=O)[C@H]2CC[C@@H](N3Cc4c(C)cnc(CN)c4NC3=O)CC2)ccc1C]

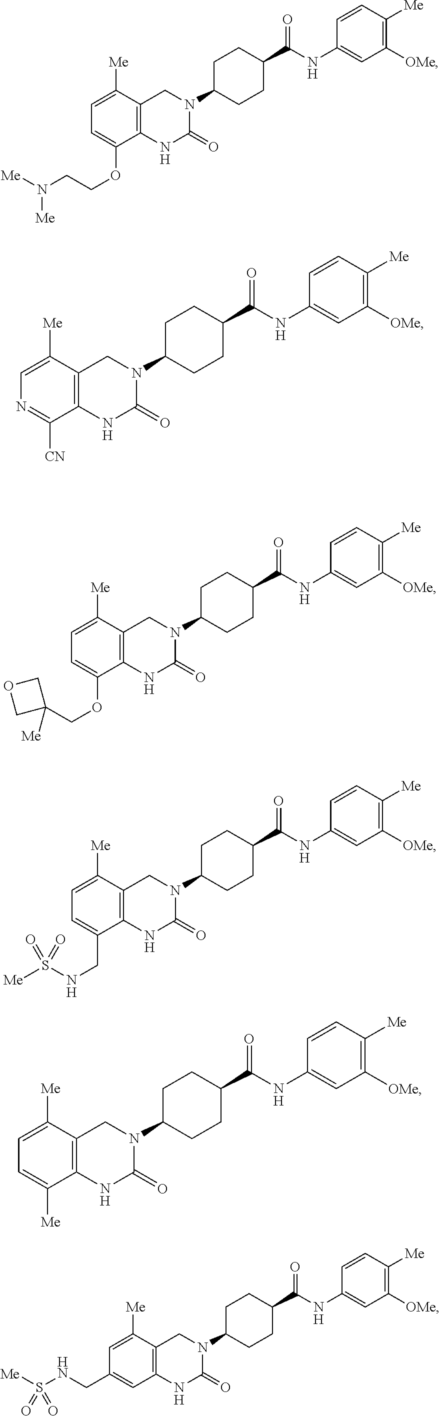 [SMILES: COc1cc(NC(=O)[C@H]2CC[C@@H](N3Cc4c(C)ccc(OCCN(C)C)c4NC3=O)CC2)ccc1C ; COc1cc(NC(=O)[C@H]2CC[C@@H](N3Cc4c(C)cnc(C#N)c4NC3=O)CC2)ccc1C ; COc1cc(NC(=O)[C@H]2CC[C@@H](N3Cc4c(C)ccc(OCC5(C)COC5)c4NC3=O)CC2)ccc1C ; COc1cc(NC(=O)[C@H]2CC[C@@H](N3Cc4c(C)ccc(CNS(C)(=O)=O)c4NC3=O)CC2)ccc1C ; COc1cc(NC(=O)[C@H]2CC[C@@H](N3Cc4c(C)ccc(C)c4NC3=O)CC2)ccc1C ; COc1cc(NC(=O)[C@H]2CC[C@@H](N3Cc4c(C)cc(CNS(C)(=O)=O)cc4NC3=O)CC2)ccc1C]

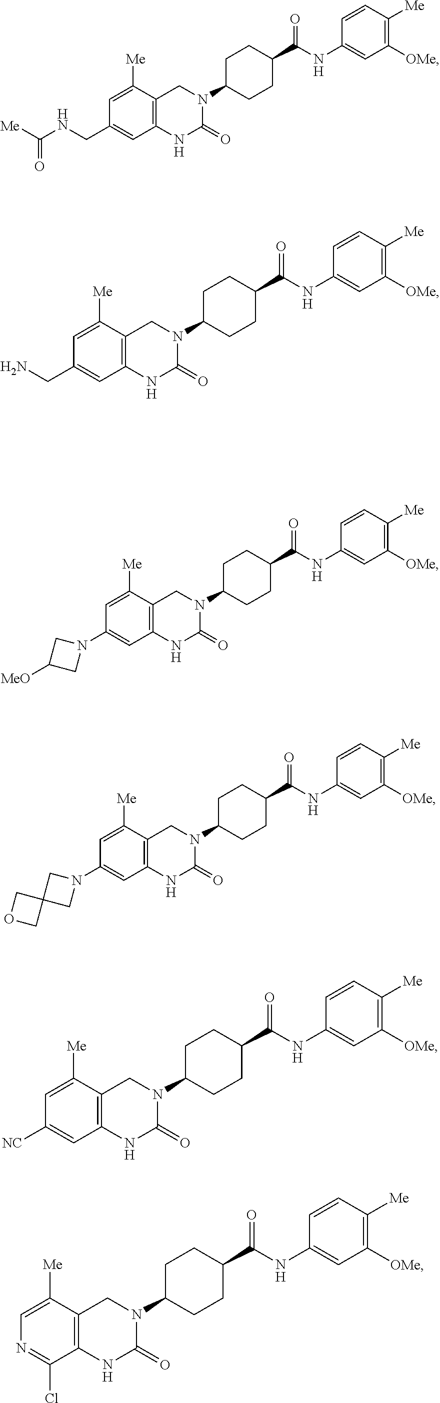 [SMILES: COc1cc(NC(=O)[C@H]2CC[C@@H](N3Cc4c(C)cc(CNC(C)=O)cc4NC3=O)CC2)ccc1C ; COc1cc(NC(=O)[C@H]2CC[C@@H](N3Cc4c(C)cc(CN)cc4NC3=O)CC2)ccc1C ; COc1cc(NC(=O)[C@H]2CC[C@@H](N3Cc4c(C)cc(N5CC(OC)C5)cc4NC3=O)CC2)ccc1C ; COc1cc(NC(=O)[C@H]2CC[C@@H](N3Cc4c(C)cc(N5CC6(COC6)C5)cc4NC3=O)CC2)ccc1C ; COc1cc(NC(=O)[C@H]2CC[C@@H](N3Cc4c(C)cc(C#N)cc4NC3=O)CC2)ccc1C ; COc1cc(NC(=O)[C@H]2CC[C@@H](N3Cc4c(C)cnc(Cl)c4NC3=O)CC2)ccc1C]

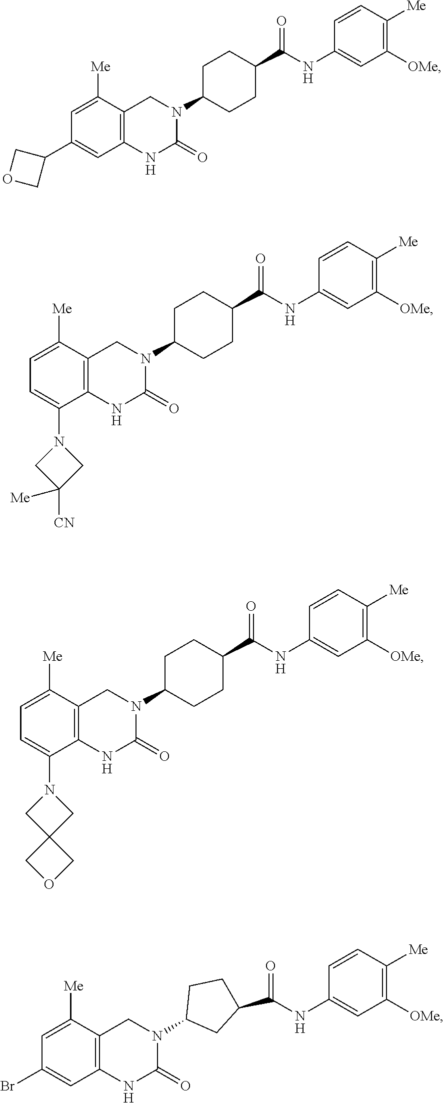 [SMILES: COc1cc(NC(=O)[C@H]2CC[C@@H](N3Cc4c(C)cc(C5COC5)cc4NC3=O)CC2)ccc1C ; COc1cc(NC(=O)[C@H]2CC[C@@H](N3Cc4c(C)ccc(N5CC(C)(C#N)C5)c4NC3=O)CC2)ccc1C ; COc1cc(NC(=O)[C@H]2CC[C@@H](N3Cc4c(C)ccc(N5CC6(COC6)C5)c4NC3=O)CC2)ccc1C ; COc1cc(NC(=O)[C@@H]2CC[C@@H](N3Cc4c(C)cc(Br)cc4NC3=O)C2)ccc1C]

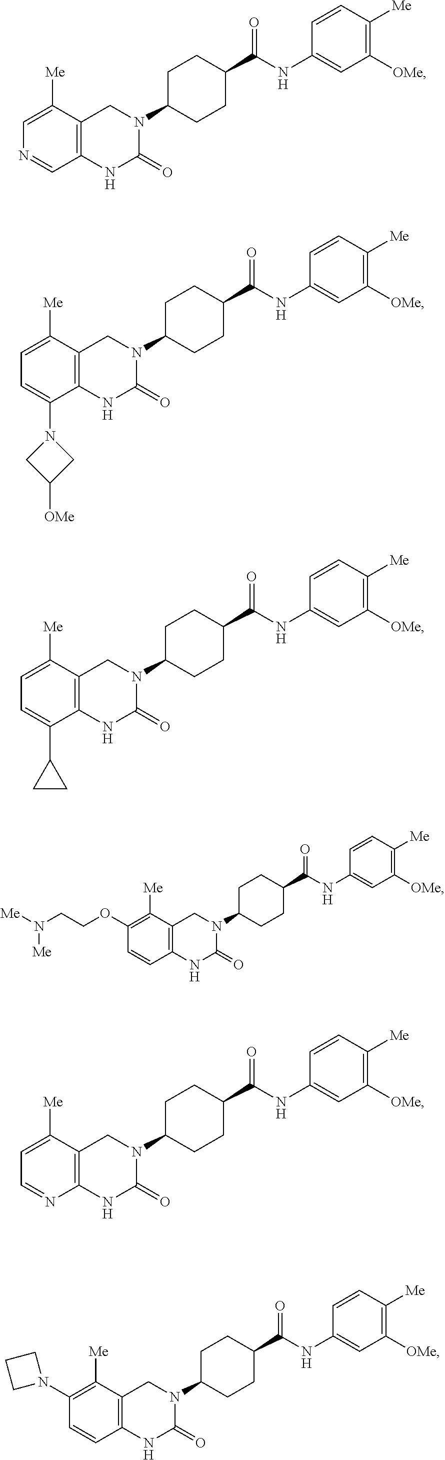 [SMILES: COc1cc(NC(=O)[C@H]2CC[C@@H](N3Cc4c(C)cncc4NC3=O)CC2)ccc1C ; COc1cc(NC(=O)[C@H]2CC[C@@H](N3Cc4c(C)ccc(N5CC(OC)C5)c4NC3=O)CC2)ccc1C ; COc1cc(NC(=O)[C@H]2CC[C@@H](N3Cc4c(C)ccc(C5CC5)c4NC3=O)CC2)ccc1C ; COc1cc(NC(=O)[C@H]2CC[C@@H](N3Cc4c(ccc(OCCN(C)C)c4C)NC3=O)CC2)ccc1C ; COc1cc(NC(=O)[C@H]2CC[C@@H](N3Cc4c(C)ccnc4NC3=O)CC2)ccc1C ; COc1cc(NC(=O)[C@H]2CC[C@@H](N3Cc4c(ccc(N5CCC5)c4C)NC3=O)CC2)ccc1C]

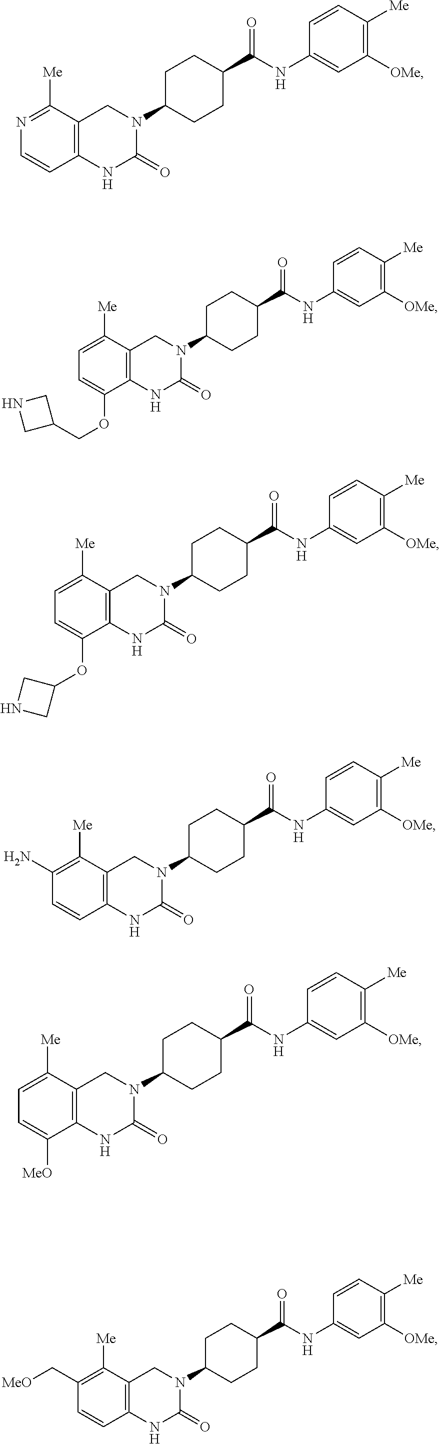 [SMILES: COc1cc(NC(=O)[C@H]2CC[C@@H](N3Cc4c(ccnc4C)NC3=O)CC2)ccc1C ; COc1cc(NC(=O)[C@H]2CC[C@@H](N3Cc4c(C)ccc(OCC5CNC5)c4NC3=O)CC2)ccc1C ; COc1cc(NC(=O)[C@H]2CC[C@@H](N3Cc4c(C)ccc(OC5CNC5)c4NC3=O)CC2)ccc1C ; COc1cc(NC(=O)[C@H]2CC[C@@H](N3Cc4c(ccc(N)c4C)NC3=O)CC2)ccc1C ; COc1cc(NC(=O)[C@H]2CC[C@@H](N3Cc4c(C)ccc(OC)c4NC3=O)CC2)ccc1C ; COCc1ccc2c(c1C)CN([C@H]1CC[C@@H](C(=O)Nc3ccc(C)c(OC)c3)CC1)C(=O)N2]

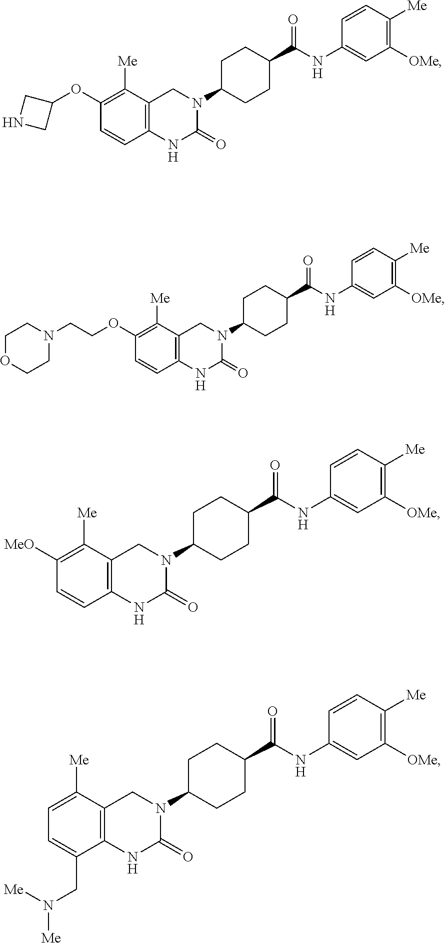 [SMILES: COc1cc(NC(=O)[C@H]2CC[C@@H](N3Cc4c(ccc(OC5CNC5)c4C)NC3=O)CC2)ccc1C ; COc1cc(NC(=O)[C@H]2CC[C@@H](N3Cc4c(ccc(OCCN5CCOCC5)c4C)NC3=O)CC2)ccc1C ; COc1cc(NC(=O)[C@H]2CC[C@@H](N3Cc4c(ccc(OC)c4C)NC3=O)CC2)ccc1C ; COc1cc(NC(=O)[C@H]2CC[C@@H](N3Cc4c(C)ccc(CN(C)C)c4NC3=O)CC2)ccc1C]

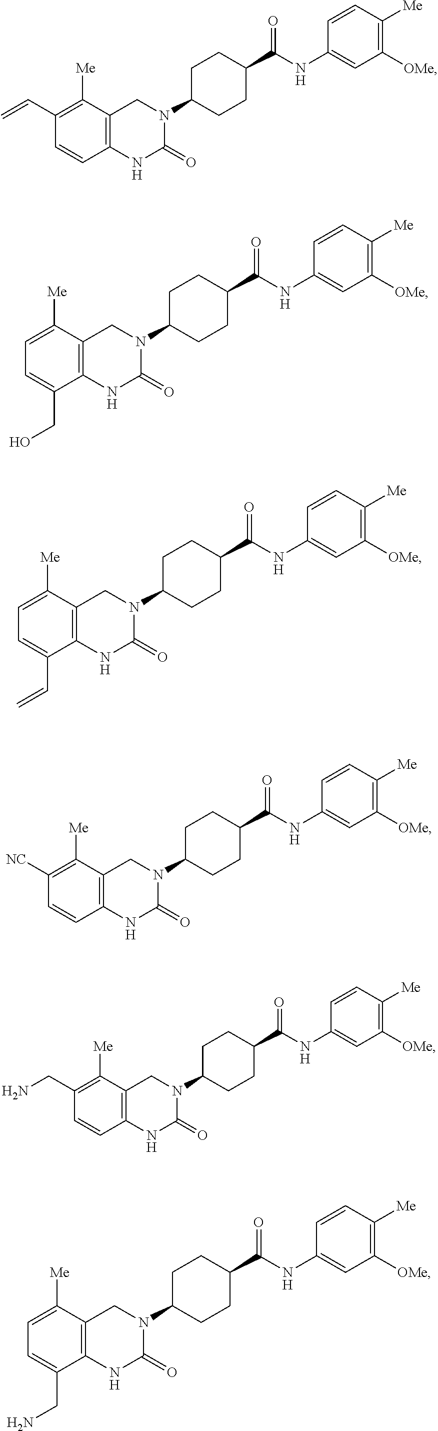 [SMILES: C=Cc1ccc2c(c1C)CN([C@H]1CC[C@@H](C(=O)Nc3ccc(C)c(OC)c3)CC1)C(=O)N2 ; COc1cc(NC(=O)[C@H]2CC[C@@H](N3Cc4c(C)ccc(CO)c4NC3=O)CC2)ccc1C ; C=Cc1ccc(C)c2c1NC(=O)N([C@H]1CC[C@@H](C(=O)Nc3ccc(C)c(OC)c3)CC1)C2 ; COc1cc(NC(=O)[C@H]2CC[C@@H](N3Cc4c(ccc(C#N)c4C)NC3=O)CC2)ccc1C ; COc1cc(NC(=O)[C@H]2CC[C@@H](N3Cc4c(ccc(CN)c4C)NC3=O)CC2)ccc1C ; COc1cc(NC(=O)[C@H]2CC[C@@H](N3Cc4c(C)ccc(CN)c4NC3=O)CC2)ccc1C]

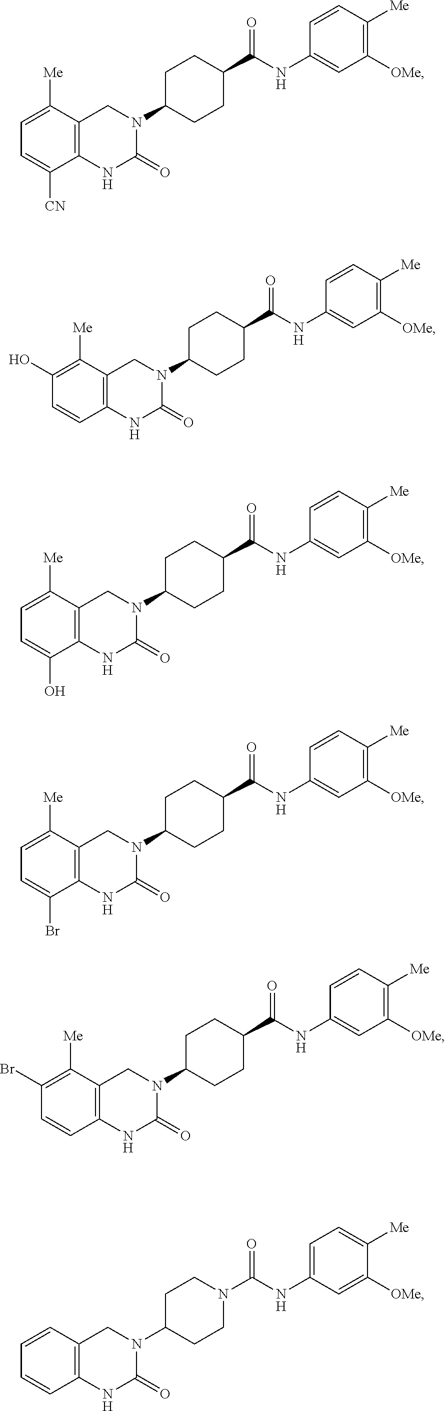 [SMILES: COc1cc(NC(=O)[C@H]2CC[C@@H](N3Cc4c(C)ccc(C#N)c4NC3=O)CC2)ccc1C ; COc1cc(NC(=O)[C@H]2CC[C@@H](N3Cc4c(ccc(O)c4C)NC3=O)CC2)ccc1C ; COc1cc(NC(=O)[C@H]2CC[C@@H](N3Cc4c(C)ccc(O)c4NC3=O)CC2)ccc1C ; COc1cc(NC(=O)[C@H]2CC[C@@H](N3Cc4c(C)ccc(Br)c4NC3=O)CC2)ccc1C ; COc1cc(NC(=O)[C@H]2CC[C@@H](N3Cc4c(ccc(Br)c4C)NC3=O)CC2)ccc1C ; COc1cc(NC(=O)N2CCC(N3Cc4ccccc4NC3=O)CC2)ccc1C]

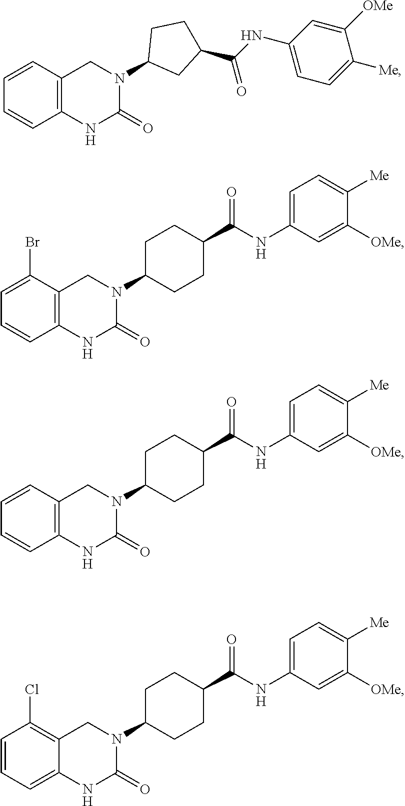 [SMILES: COc1cc(NC(=O)[C@@H]2CC[C@H](N3Cc4ccccc4NC3=O)C2)ccc1C ; COc1cc(NC(=O)[C@H]2CC[C@@H](N3Cc4c(Br)cccc4NC3=O)CC2)ccc1C ; COc1cc(NC(=O)[C@H]2CC[C@@H](N3Cc4ccccc4NC3=O)CC2)ccc1C ; COc1cc(NC(=O)[C@H]2CC[C@@H](N3Cc4c(Cl)cccc4NC3=O)CC2)ccc1C]

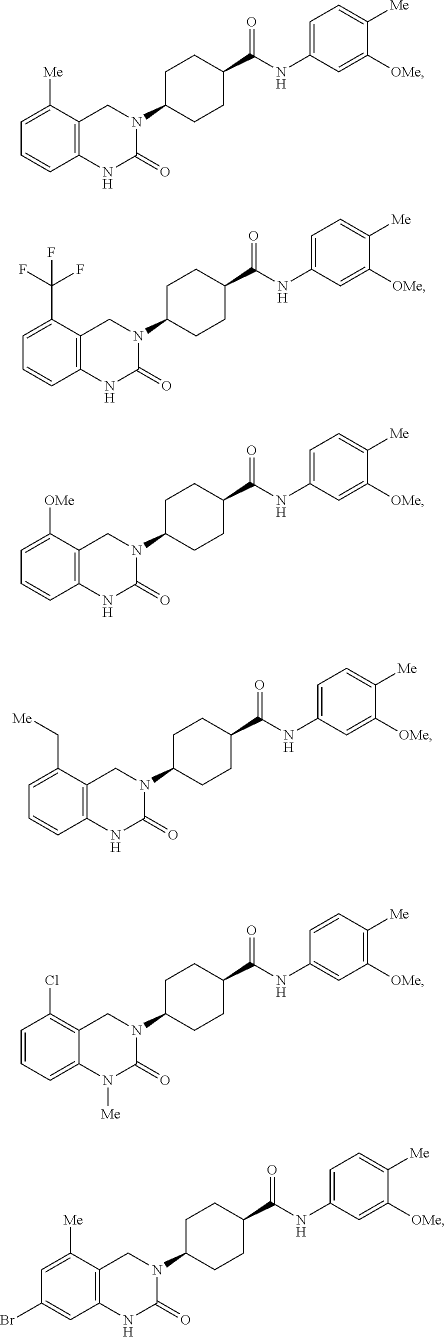 [SMILES: COc1cc(NC(=O)[C@H]2CC[C@@H](N3Cc4c(C)cccc4NC3=O)CC2)ccc1C ; COc1cc(NC(=O)[C@H]2CC[C@@H](N3Cc4c(cccc4C(F)(F)F)NC3=O)CC2)ccc1C ; COc1cc(NC(=O)[C@H]2CC[C@@H](N3Cc4c(cccc4OC)NC3=O)CC2)ccc1C ; CCc1cccc2c1CN([C@H]1CC[C@@H](C(=O)Nc3ccc(C)c(OC)c3)CC1)C(=O)N2 ; COc1cc(NC(=O)[C@H]2CC[C@@H](N3Cc4c(Cl)cccc4N(C)C3=O)CC2)ccc1C ; COc1cc(NC(=O)[C@H]2CC[C@@H](N3Cc4c(C)cc(Br)cc4NC3=O)CC2)ccc1C]

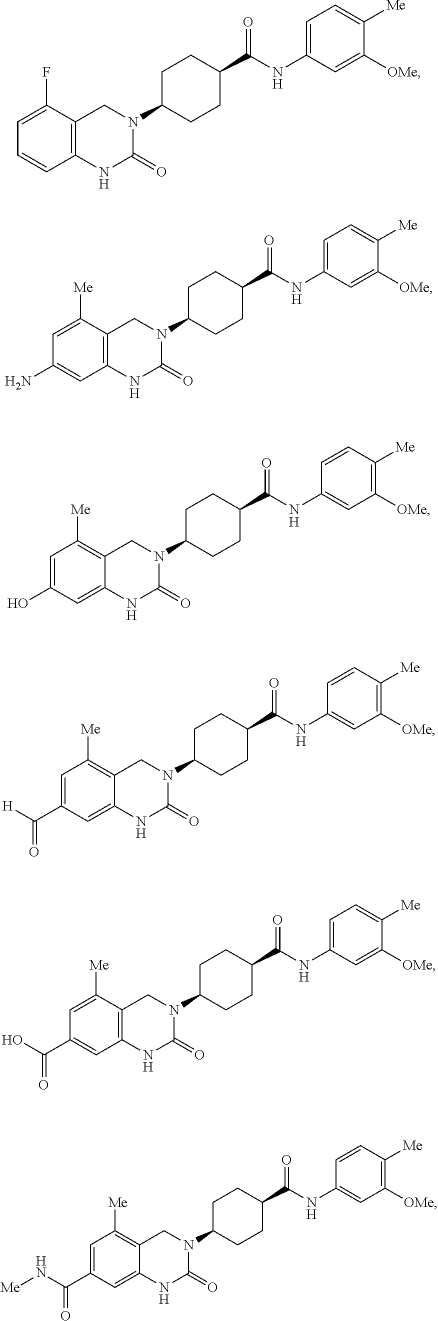 [SMILES: COc1cc(NC(=O)[C@H]2CC[C@@H](N3Cc4c(F)cccc4NC3=O)CC2)ccc1C ; COc1cc(NC(=O)[C@H]2CC[C@@H](N3Cc4c(C)cc(N)cc4NC3=O)CC2)ccc1C ; COc1cc(NC(=O)[C@H]2CC[C@@H](N3Cc4c(C)cc(O)cc4NC3=O)CC2)ccc1C ; COc1cc(NC(=O)[C@H]2CC[C@@H](N3Cc4c(C)cc(C=O)cc4NC3=O)CC2)ccc1C ; COc1cc(NC(=O)[C@H]2CC[C@@H](N3Cc4c(C)cc(C(=O)O)cc4NC3=O)CC2)ccc1C ; CNC(=O)c1cc(C)c2c(c1)NC(=O)N([C@H]1CC[C@@H](C(=O)Nc3ccc(C)c(OC)c3)CC1)C2]

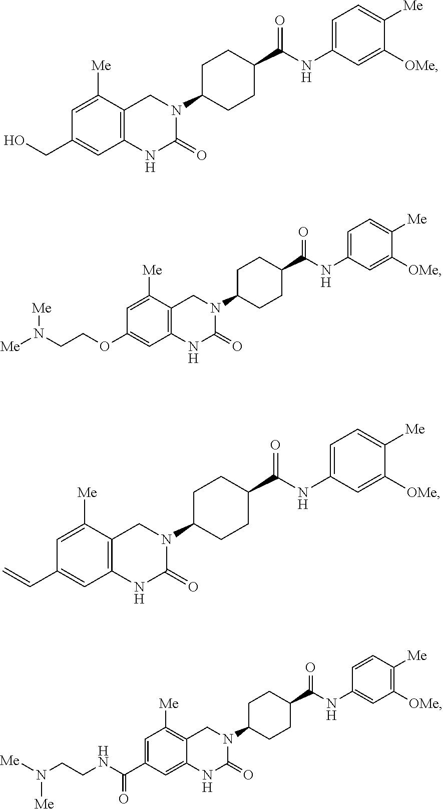 [SMILES: COc1cc(NC(=O)[C@H]2CC[C@@H](N3Cc4c(C)cc(CO)cc4NC3=O)CC2)ccc1C ; COc1cc(NC(=O)[C@H]2CC[C@@H](N3Cc4c(C)cc(OCCN(C)C)cc4NC3=O)CC2)ccc1C ; C=Cc1cc(C)c2c(c1)NC(=O)N([C@H]1CC[C@@H](C(=O)Nc3ccc(C)c(OC)c3)CC1)C2 ; COc1cc(NC(=O)[C@H]2CC[C@@H](N3Cc4c(C)cc(C(=O)NCCN(C)C)cc4NC3=O)CC2)ccc1C]

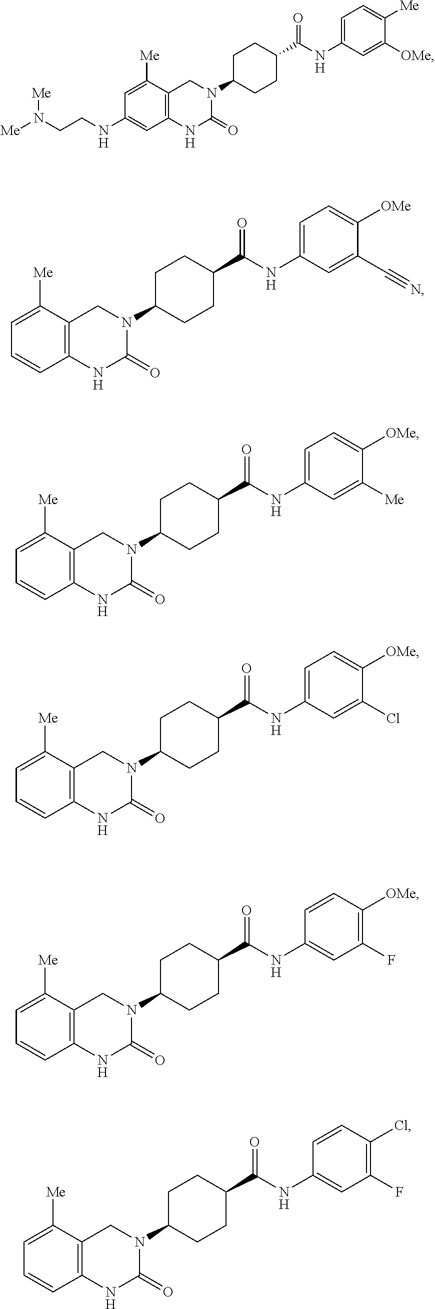 [SMILES: COc1cc(NC(=O)[C@H]2CC[C@H](N3Cc4c(C)cc(NCCN(C)C)cc4NC3=O)CC2)ccc1C ; COc1ccc(NC(=O)[C@H]2CC[C@@H](N3Cc4c(C)cccc4NC3=O)CC2)cc1C#N ; COc1ccc(NC(=O)[C@H]2CC[C@@H](N3Cc4c(C)cccc4NC3=O)CC2)cc1C ; COc1ccc(NC(=O)[C@H]2CC[C@@H](N3Cc4c(C)cccc4NC3=O)CC2)cc1Cl ; COc1ccc(NC(=O)[C@H]2CC[C@@H](N3Cc4c(C)cccc4NC3=O)CC2)cc1F ; Cc1cccc2c1CN([C@H]1CC[C@@H](C(=O)Nc3ccc(Cl)c(F)c3)CC1)C(=O)N2]

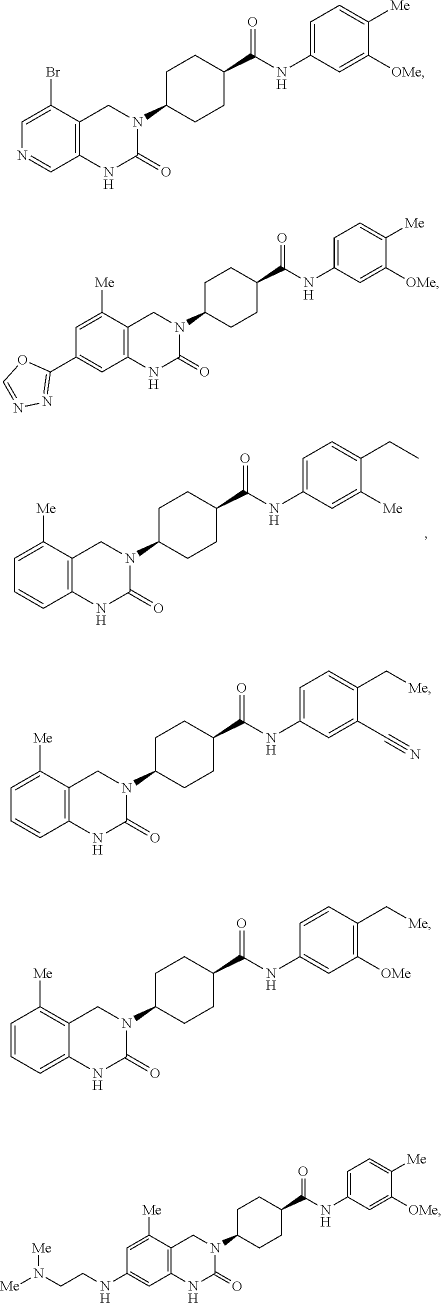 [SMILES: COc1cc(NC(=O)[C@H]2CC[C@@H](N3Cc4c(Br)cncc4NC3=O)CC2)ccc1C ; COc1cc(NC(=O)[C@H]2CC[C@@H](N3Cc4c(C)cc(-c5nnco5)cc4NC3=O)CC2)ccc1C ; CCc1ccc(NC(=O)[C@H]2CC[C@@H](N3Cc4c(C)cccc4NC3=O)CC2)cc1C ; CCc1ccc(NC(=O)[C@H]2CC[C@@H](N3Cc4c(C)cccc4NC3=O)CC2)cc1C#N ; CCc1ccc(NC(=O)[C@H]2CC[C@@H](N3Cc4c(C)cccc4NC3=O)CC2)cc1OC ; COc1cc(NC(=O)[C@H]2CC[C@@H](N3Cc4c(C)cc(NCCN(C)C)cc4NC3=O)CC2)ccc1C]

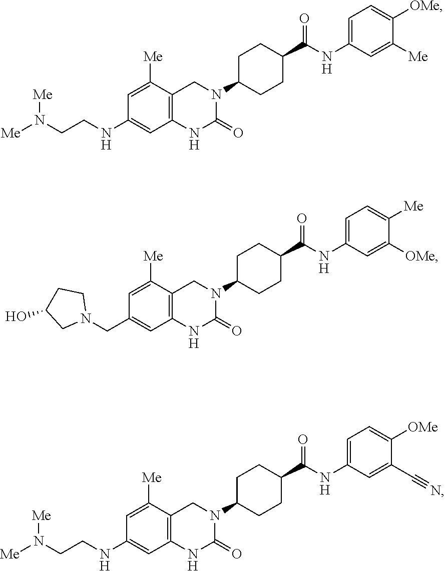 [SMILES: COc1ccc(NC(=O)[C@H]2CC[C@@H](N3Cc4c(C)cc(NCCN(C)C)cc4NC3=O)CC2)cc1C ; COc1cc(NC(=O)[C@H]2CC[C@@H](N3Cc4c(C)cc(CN5CC[C@@H](O)C5)cc4NC3=O)CC2)ccc1C ; COc1ccc(NC(=O)[C@H]2CC[C@@H](N3Cc4c(C)cc(NCCN(C)C)cc4NC3=O)CC2)cc1C#N]

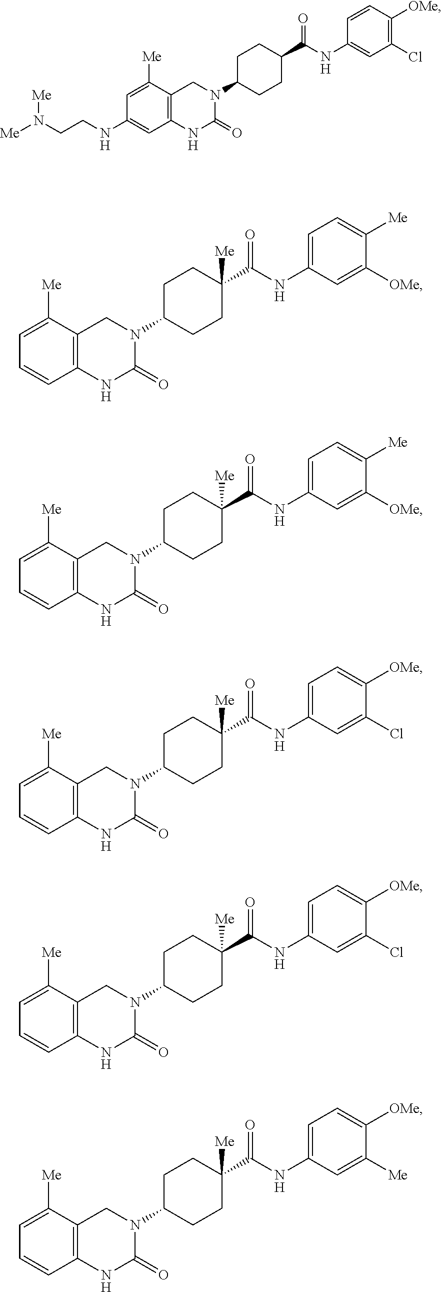 [SMILES: COc1ccc(NC(=O)[C@H]2CC[C@@H](N3Cc4c(C)cc(NCCN(C)C)cc4NC3=O)CC2)cc1Cl ; COc1cc(NC(=O)[C@]2(C)CC[C@H](N3Cc4c(C)cccc4NC3=O)CC2)ccc1C ; COc1cc(NC(=O)[C@]2(C)CC[C@@H](N3Cc4c(C)cccc4NC3=O)CC2)ccc1C ; COc1ccc(NC(=O)[C@]2(C)CC[C@H](N3Cc4c(C)cccc4NC3=O)CC2)cc1Cl ; COc1ccc(NC(=O)[C@]2(C)CC[C@@H](N3Cc4c(C)cccc4NC3=O)CC2)cc1Cl ; COc1ccc(NC(=O)[C@]2(C)CC[C@H](N3Cc4c(C)cccc4NC3=O)CC2)cc1C]

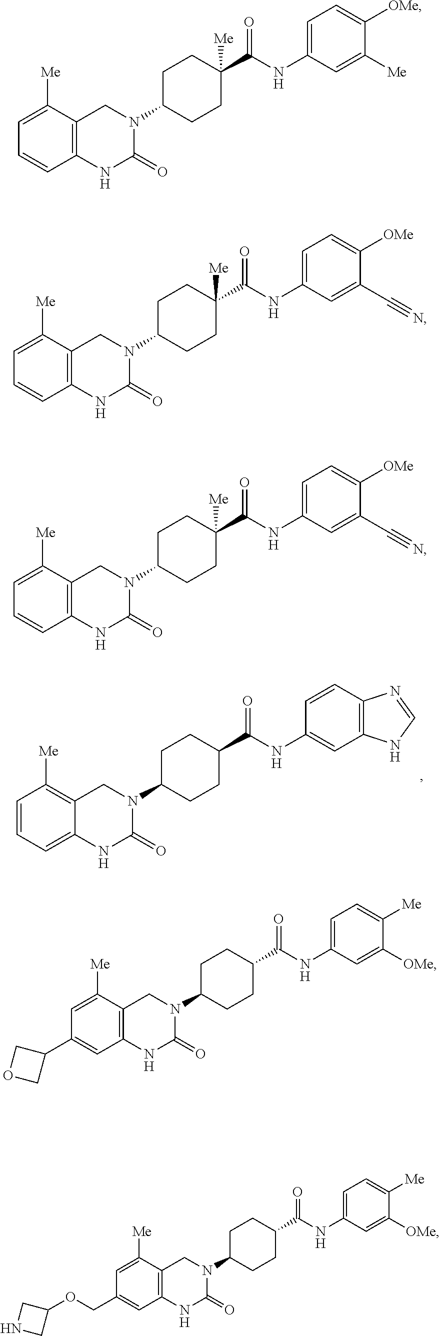 [SMILES: COc1ccc(NC(=O)[C@]2(C)CC[C@@H](N3Cc4c(C)cccc4NC3=O)CC2)cc1C ; COc1ccc(NC(=O)[C@]2(C)CC[C@H](N3Cc4c(C)cccc4NC3=O)CC2)cc1C#N ; COc1ccc(NC(=O)[C@]2(C)CC[C@@H](N3Cc4c(C)cccc4NC3=O)CC2)cc1C#N ; Cc1cccc2c1CN([C@H]1CC[C@@H](C(=O)Nc3ccc4nc[nH]c4c3)CC1)C(=O)N2 ; COc1cc(NC(=O)[C@H]2CC[C@H](N3Cc4c(C)cc(C5COC5)cc4NC3=O)CC2)ccc1C ; COc1cc(NC(=O)[C@H]2CC[C@H](N3Cc4c(C)cc(COC5CNC5)cc4NC3=O)CC2)ccc1C]

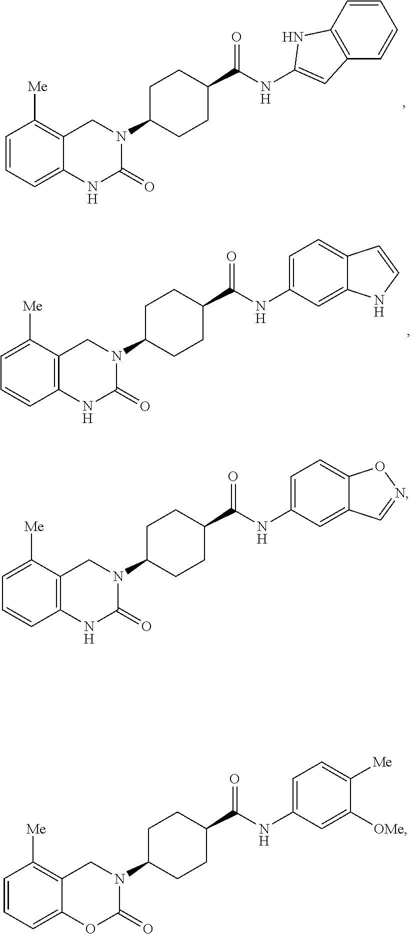 [SMILES: Cc1cccc2c1CN([C@H]1CC[C@@H](C(=O)Nc3cc4ccccc4[nH]3)CC1)C(=O)N2 ; Cc1cccc2c1CN([C@H]1CC[C@@H](C(=O)Nc3ccc4cc[nH]c4c3)CC1)C(=O)N2 ; Cc1cccc2c1CN([C@H]1CC[C@@H](C(=O)Nc3ccc4oncc4c3)CC1)C(=O)N2 ; COc1cc(NC(=O)[C@H]2CC[C@@H](N3Cc4c(C)cccc4OC3=O)CC2)ccc1C]

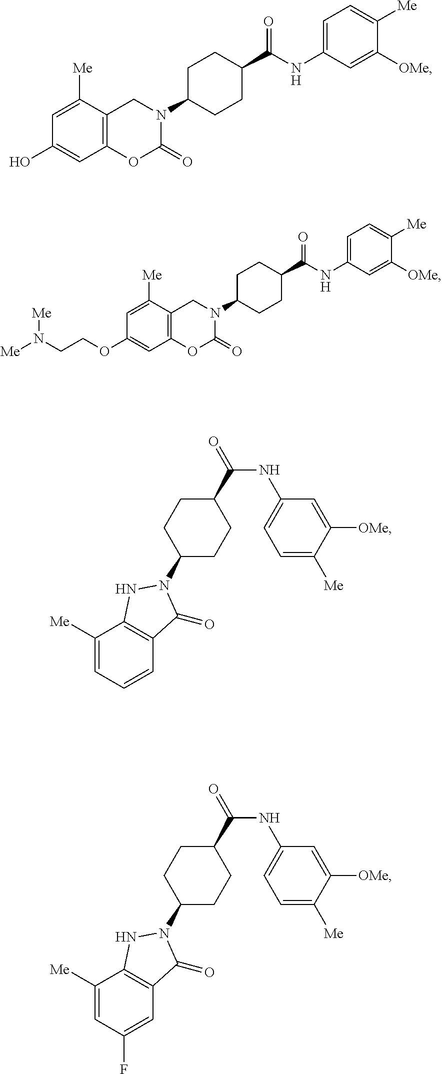 [SMILES: COc1cc(NC(=O)[C@H]2CC[C@@H](N3Cc4c(C)cc(O)cc4OC3=O)CC2)ccc1C ; COc1cc(NC(=O)[C@H]2CC[C@@H](N3Cc4c(C)cc(OCCN(C)C)cc4OC3=O)CC2)ccc1C ; COc1cc(NC(=O)[C@H]2CC[C@@H](n3[nH]c4c(C)cccc4c3=O)CC2)ccc1C ; COc1cc(NC(=O)[C@H]2CC[C@@H](n3[nH]c4c(C)cc(F)cc4c3=O)CC2)ccc1C]

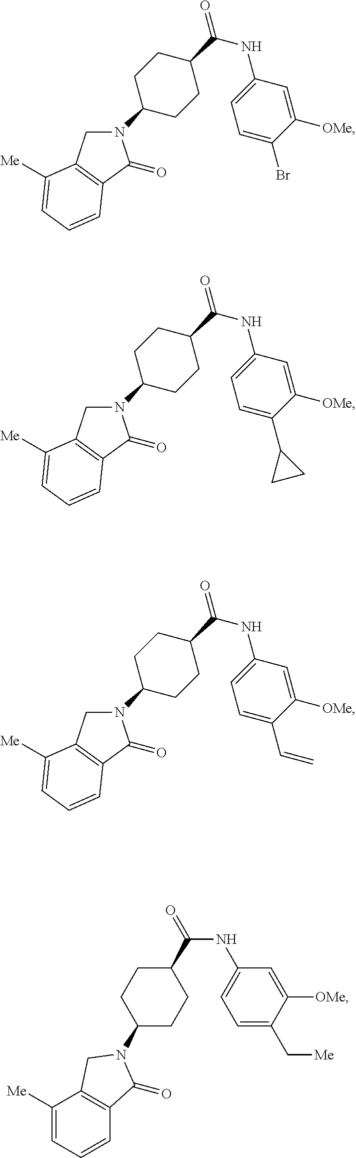 [SMILES: COc1cc(NC(=O)[C@H]2CC[C@@H](N3Cc4c(C)cccc4C3=O)CC2)ccc1Br ; COc1cc(NC(=O)[C@H]2CC[C@@H](N3Cc4c(C)cccc4C3=O)CC2)ccc1C1CC1 ; C=Cc1ccc(NC(=O)[C@H]2CC[C@@H](N3Cc4c(C)cccc4C3=O)CC2)cc1OC ; CCc1ccc(NC(=O)[C@H]2CC[C@@H](N3Cc4c(C)cccc4C3=O)CC2)cc1OC]

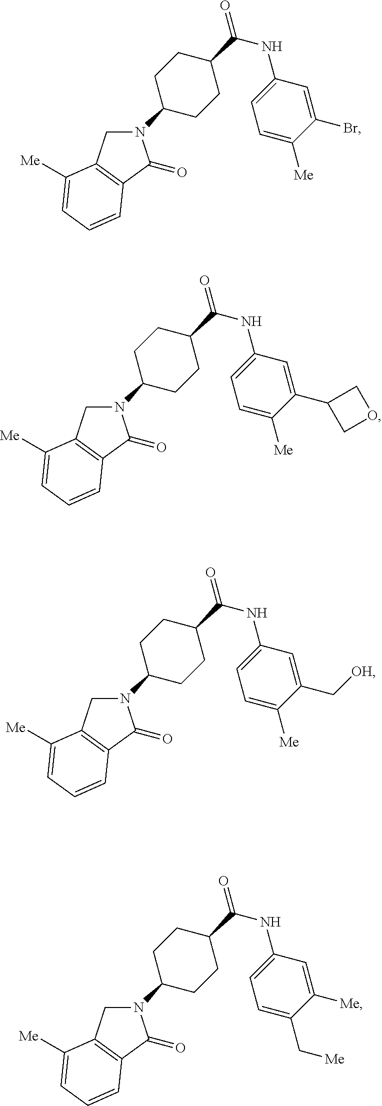 [SMILES: Cc1ccc(NC(=O)[C@H]2CC[C@@H](N3Cc4c(C)cccc4C3=O)CC2)cc1Br ; Cc1ccc(NC(=O)[C@H]2CC[C@@H](N3Cc4c(C)cccc4C3=O)CC2)cc1C1COC1 ; Cc1ccc(NC(=O)[C@H]2CC[C@@H](N3Cc4c(C)cccc4C3=O)CC2)cc1CO ; CCc1ccc(NC(=O)[C@H]2CC[C@@H](N3Cc4c(C)cccc4C3=O)CC2)cc1C]

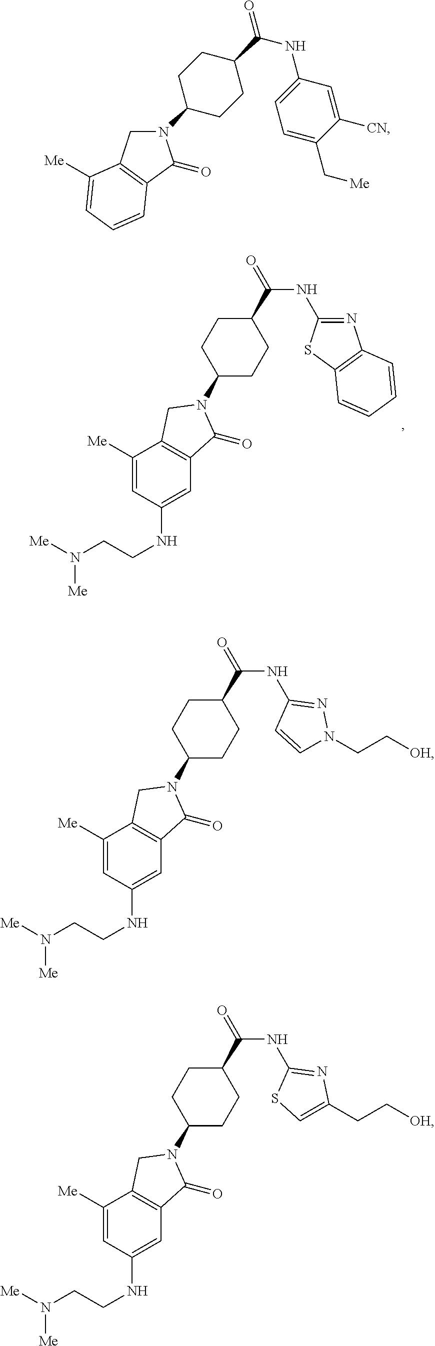 [SMILES: CCc1ccc(NC(=O)[C@H]2CC[C@@H](N3Cc4c(C)cccc4C3=O)CC2)cc1C#N ; Cc1cc(NCCN(C)C)cc2c1CN([C@H]1CC[C@@H](C(=O)Nc3nc4ccccc4s3)CC1)C2=O ; Cc1cc(NCCN(C)C)cc2c1CN([C@H]1CC[C@@H](C(=O)Nc3ccn(CCO)n3)CC1)C2=O ; Cc1cc(NCCN(C)C)cc2c1CN([C@H]1CC[C@@H](C(=O)Nc3nc(CCO)cs3)CC1)C2=O]

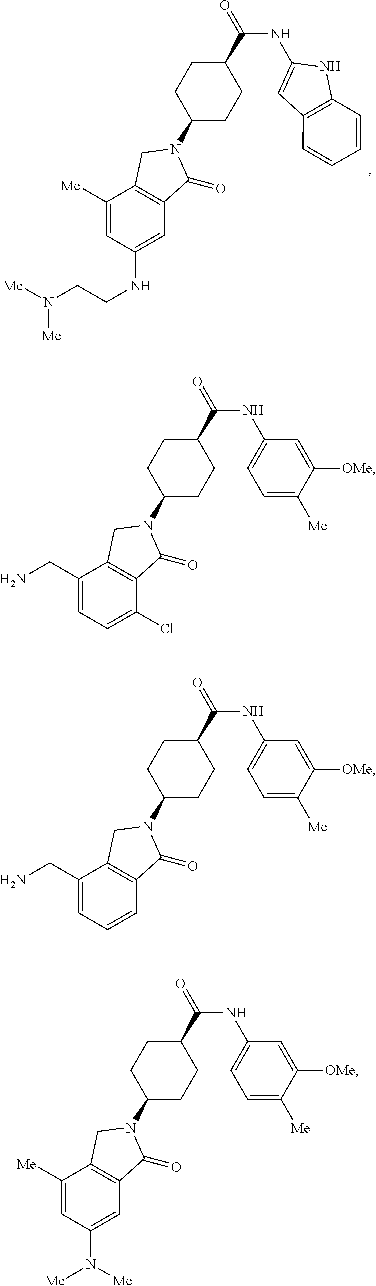 [SMILES: Cc1cc(NCCN(C)C)cc2c1CN([C@H]1CC[C@@H](C(=O)Nc3cc4ccccc4[nH]3)CC1)C2=O ; COc1cc(NC(=O)[C@H]2CC[C@@H](N3Cc4c(CN)ccc(Cl)c4C3=O)CC2)ccc1C ; COc1cc(NC(=O)[C@H]2CC[C@@H](N3Cc4c(CN)cccc4C3=O)CC2)ccc1C ; COc1cc(NC(=O)[C@H]2CC[C@@H](N3Cc4c(C)cc(N(C)C)cc4C3=O)CC2)ccc1C]

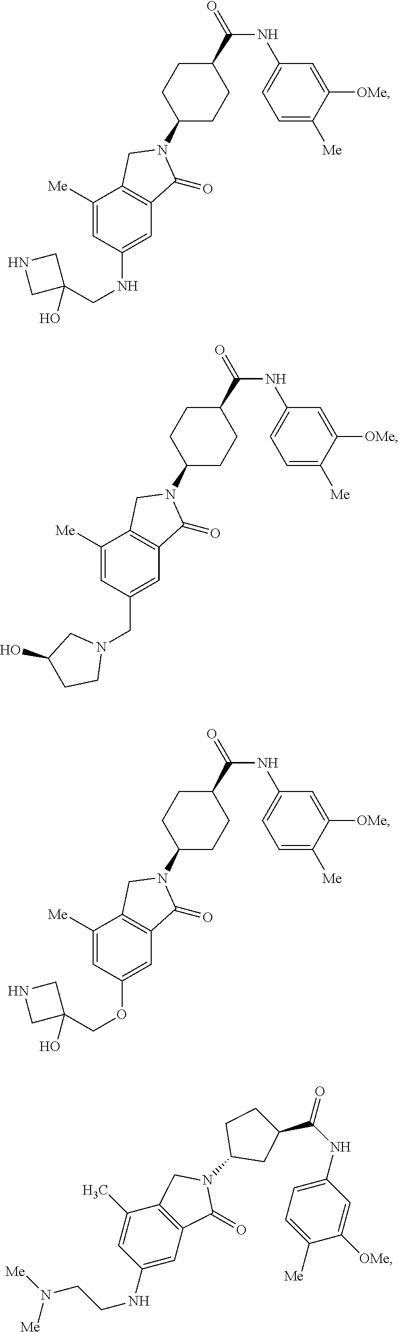 [SMILES: COc1cc(NC(=O)[C@H]2CC[C@@H](N3Cc4c(C)cc(NCC5(O)CNC5)cc4C3=O)CC2)ccc1C ; COc1cc(NC(=O)[C@H]2CC[C@@H](N3Cc4c(C)cc(CN5CC[C@@H](O)C5)cc4C3=O)CC2)ccc1C ; COc1cc(NC(=O)[C@H]2CC[C@@H](N3Cc4c(C)cc(OCC5(O)CNC5)cc4C3=O)CC2)ccc1C ; COc1cc(NC(=O)[C@@H]2CC[C@@H](N3Cc4c(C)cc(NCCN(C)C)cc4C3=O)C2)ccc1C]

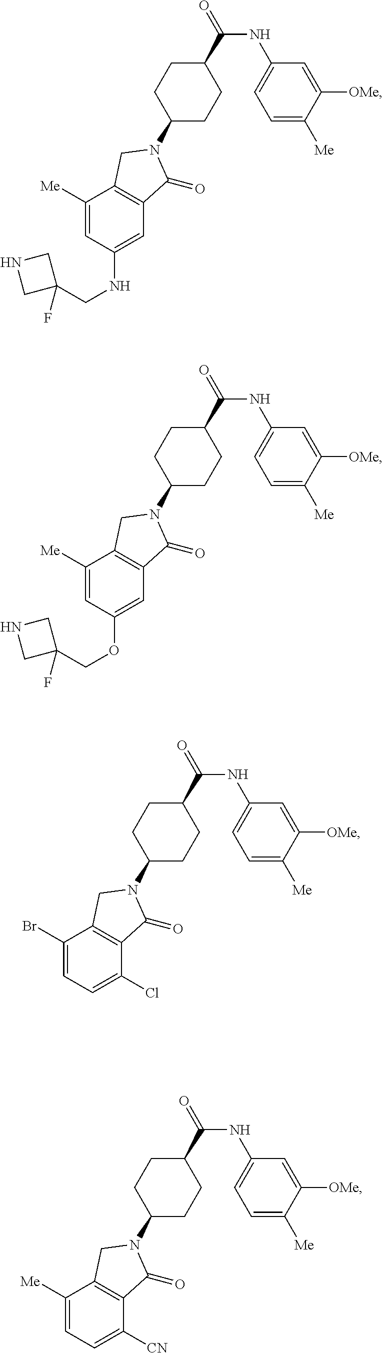 [SMILES: COc1cc(NC(=O)[C@H]2CC[C@@H](N3Cc4c(C)cc(NCC5(F)CNC5)cc4C3=O)CC2)ccc1C ; COc1cc(NC(=O)[C@H]2CC[C@@H](N3Cc4c(C)cc(OCC5(F)CNC5)cc4C3=O)CC2)ccc1C ; COc1cc(NC(=O)[C@H]2CC[C@@H](N3Cc4c(Br)ccc(Cl)c4C3=O)CC2)ccc1C ; COc1cc(NC(=O)[C@H]2CC[C@@H](N3Cc4c(C)ccc(C#N)c4C3=O)CC2)ccc1C]

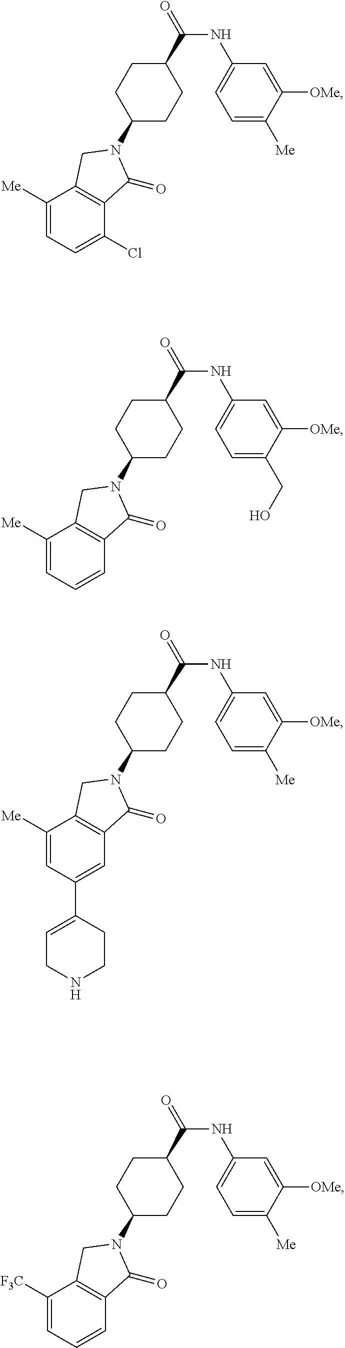 [SMILES: COc1cc(NC(=O)[C@H]2CC[C@@H](N3Cc4c(C)ccc(Cl)c4C3=O)CC2)ccc1C ; COc1cc(NC(=O)[C@H]2CC[C@@H](N3Cc4c(C)cccc4C3=O)CC2)ccc1CO ; COc1cc(NC(=O)[C@H]2CC[C@@H](N3Cc4c(C)cc(C5=CCNCC5)cc4C3=O)CC2)ccc1C ; COc1cc(NC(=O)[C@H]2CC[C@@H](N3Cc4c(cccc4C(F)(F)F)C3=O)CC2)ccc1C]

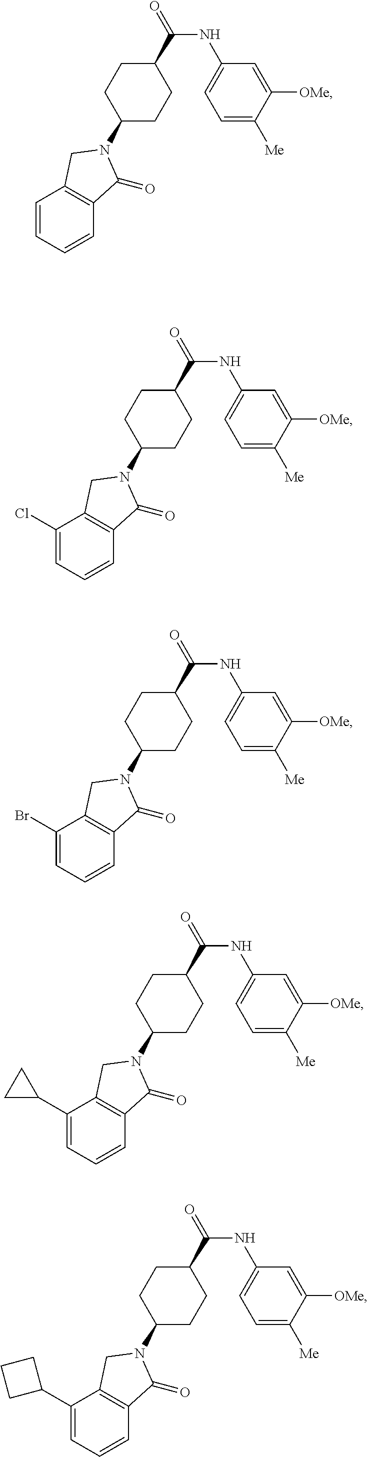 [SMILES: COc1cc(NC(=O)[C@H]2CC[C@@H](N3Cc4ccccc4C3=O)CC2)ccc1C ; COc1cc(NC(=O)[C@H]2CC[C@@H](N3Cc4c(Cl)cccc4C3=O)CC2)ccc1C ; COc1cc(NC(=O)[C@H]2CC[C@@H](N3Cc4c(Br)cccc4C3=O)CC2)ccc1C ; COc1cc(NC(=O)[C@H]2CC[C@@H](N3Cc4c(cccc4C4CC4)C3=O)CC2)ccc1C ; COc1cc(NC(=O)[C@H]2CC[C@@H](N3Cc4c(cccc4C4CCC4)C3=O)CC2)ccc1C]

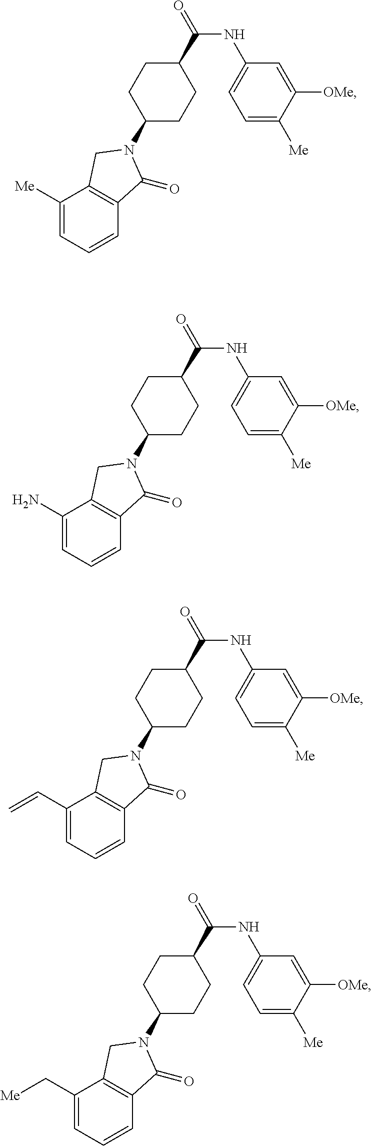 [SMILES: COc1cc(NC(=O)[C@H]2CC[C@@H](N3Cc4c(C)cccc4C3=O)CC2)ccc1C ; COc1cc(NC(=O)[C@H]2CC[C@@H](N3Cc4c(N)cccc4C3=O)CC2)ccc1C ; C=Cc1cccc2c1CN([C@H]1CC[C@@H](C(=O)Nc3ccc(C)c(OC)c3)CC1)C2=O ; CCc1cccc2c1CN([C@H]1CC[C@@H](C(=O)Nc3ccc(C)c(OC)c3)CC1)C2=O]

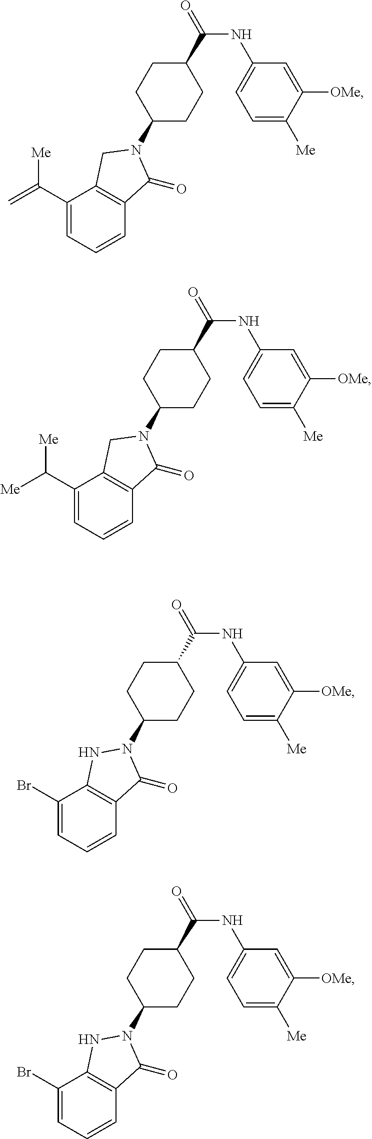 [SMILES: C=C(C)c1cccc2c1CN([C@H]1CC[C@@H](C(=O)Nc3ccc(C)c(OC)c3)CC1)C2=O ; COc1cc(NC(=O)[C@H]2CC[C@@H](N3Cc4c(cccc4C(C)C)C3=O)CC2)ccc1C ; COc1cc(NC(=O)[C@H]2CC[C@H](n3[nH]c4c(Br)cccc4c3=O)CC2)ccc1C ; COc1cc(NC(=O)[C@H]2CC[C@@H](n3[nH]c4c(Br)cccc4c3=O)CC2)ccc1C]

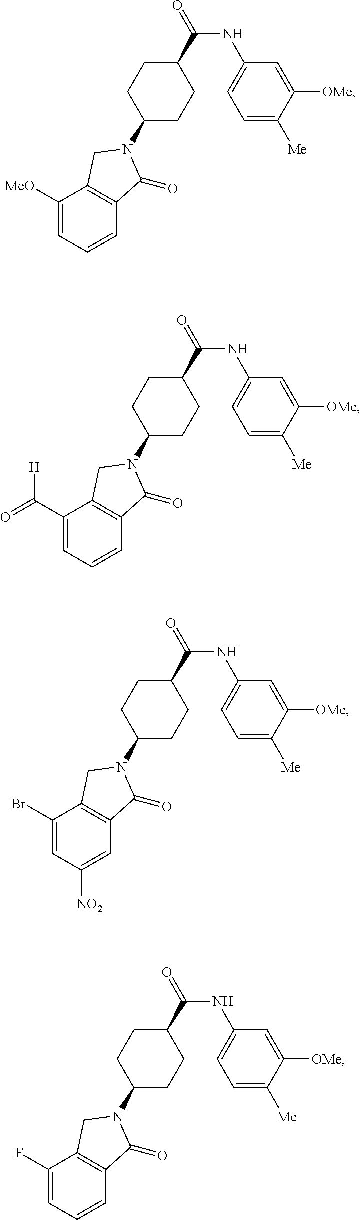 [SMILES: COc1cc(NC(=O)[C@H]2CC[C@@H](N3Cc4c(OC)cccc4C3=O)CC2)ccc1C ; COc1cc(NC(=O)[C@H]2CC[C@@H](N3Cc4c(C=O)cccc4C3=O)CC2)ccc1C ; COc1cc(NC(=O)[C@H]2CC[C@@H](N3Cc4c(Br)cc([N+](=O)[O-])cc4C3=O)CC2)ccc1C ; COc1cc(NC(=O)[C@H]2CC[C@@H](N3Cc4c(F)cccc4C3=O)CC2)ccc1C]

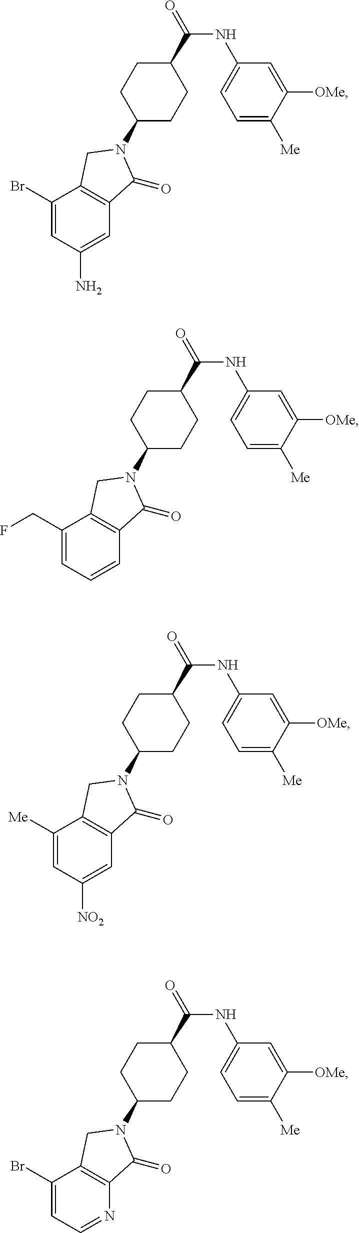 [SMILES: COc1cc(NC(=O)[C@H]2CC[C@@H](N3Cc4c(Br)cc(N)cc4C3=O)CC2)ccc1C ; COc1cc(NC(=O)[C@H]2CC[C@@H](N3Cc4c(CF)cccc4C3=O)CC2)ccc1C ; COc1cc(NC(=O)[C@H]2CC[C@@H](N3Cc4c(C)cc([N+](=O)[O-])cc4C3=O)CC2)ccc1C ; COc1cc(NC(=O)[C@H]2CC[C@@H](N3Cc4c(Br)ccnc4C3=O)CC2)ccc1C]

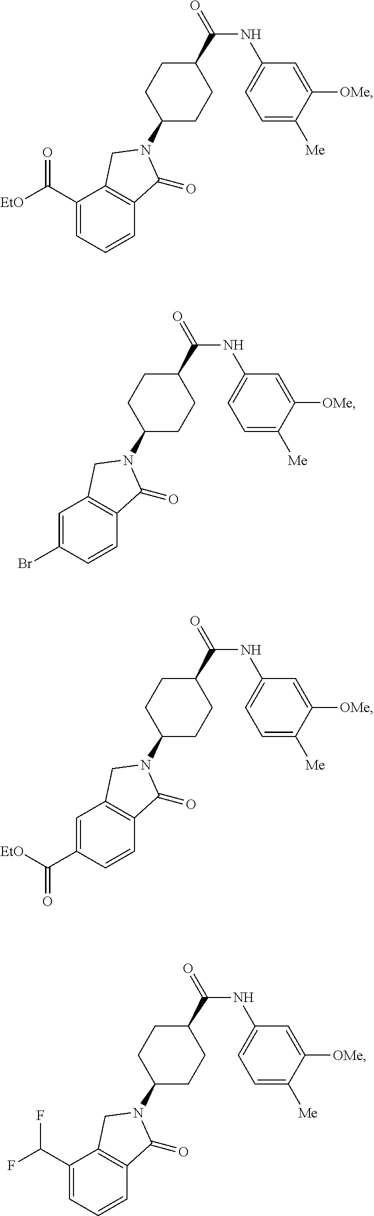 [SMILES: CCOC(=O)c1cccc2c1CN([C@H]1CC[C@@H](C(=O)Nc3ccc(C)c(OC)c3)CC1)C2=O ; COc1cc(NC(=O)[C@H]2CC[C@@H](N3Cc4cc(Br)ccc4C3=O)CC2)ccc1C ; CCOC(=O)c1ccc2c(c1)CN([C@H]1CC[C@@H](C(=O)Nc3ccc(C)c(OC)c3)CC1)C2=O ; COc1cc(NC(=O)[C@H]2CC[C@@H](N3Cc4c(cccc4C(F)F)C3=O)CC2)ccc1C]

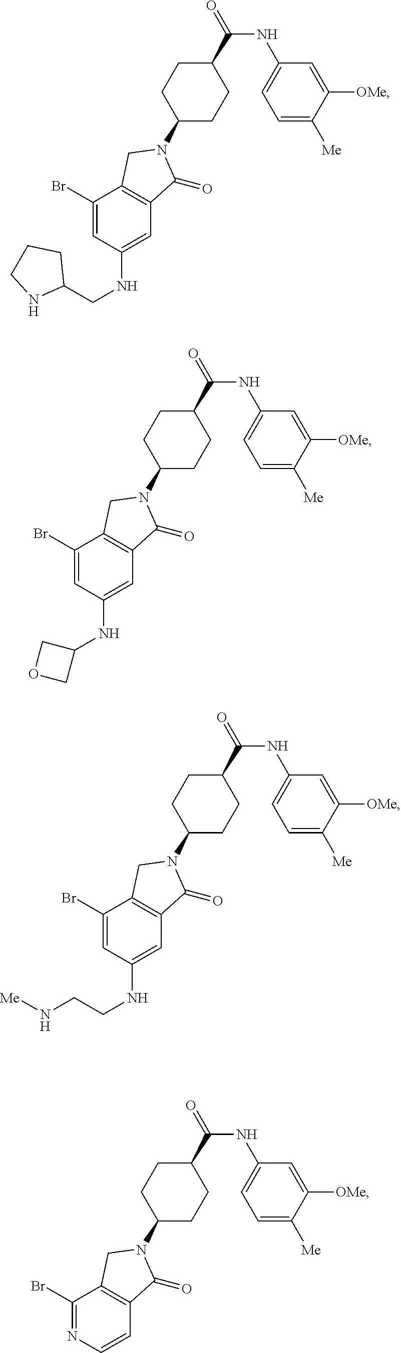 [SMILES: COc1cc(NC(=O)[C@H]2CC[C@@H](N3Cc4c(Br)cc(NCC5CCCN5)cc4C3=O)CC2)ccc1C ; COc1cc(NC(=O)[C@H]2CC[C@@H](N3Cc4c(Br)cc(NC5COC5)cc4C3=O)CC2)ccc1C ; CNCCNc1cc(Br)c2c(c1)C(=O)N([C@H]1CC[C@@H](C(=O)Nc3ccc(C)c(OC)c3)CC1)C2 ; COc1cc(NC(=O)[C@H]2CC[C@@H](N3Cc4c(ccnc4Br)C3=O)CC2)ccc1C]

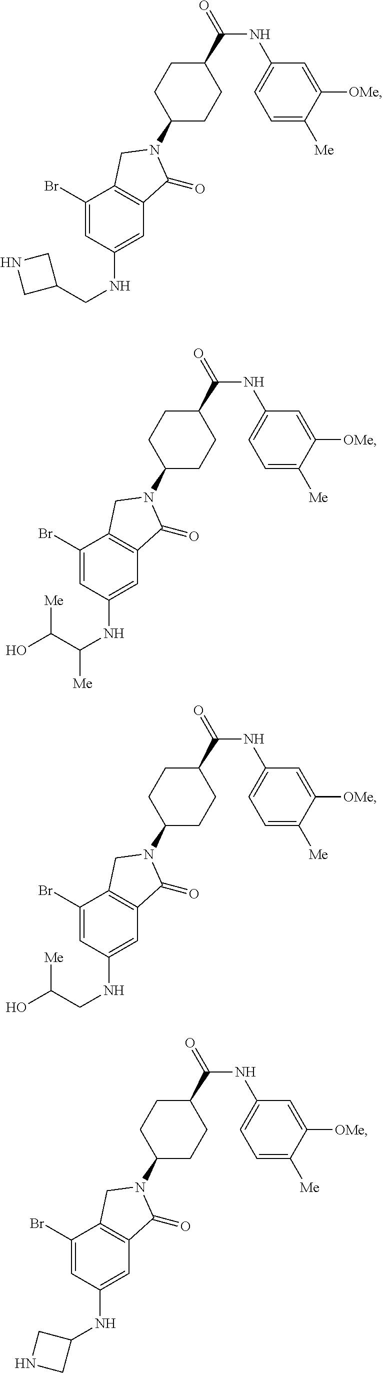 [SMILES: COc1cc(NC(=O)[C@H]2CC[C@@H](N3Cc4c(Br)cc(NCC5CNC5)cc4C3=O)CC2)ccc1C ; COc1cc(NC(=O)[C@H]2CC[C@@H](N3Cc4c(Br)cc(NC(C)C(C)O)cc4C3=O)CC2)ccc1C ; COc1cc(NC(=O)[C@H]2CC[C@@H](N3Cc4c(Br)cc(NCC(C)O)cc4C3=O)CC2)ccc1C ; COc1cc(NC(=O)[C@H]2CC[C@@H](N3Cc4c(Br)cc(NC5CNC5)cc4C3=O)CC2)ccc1C]

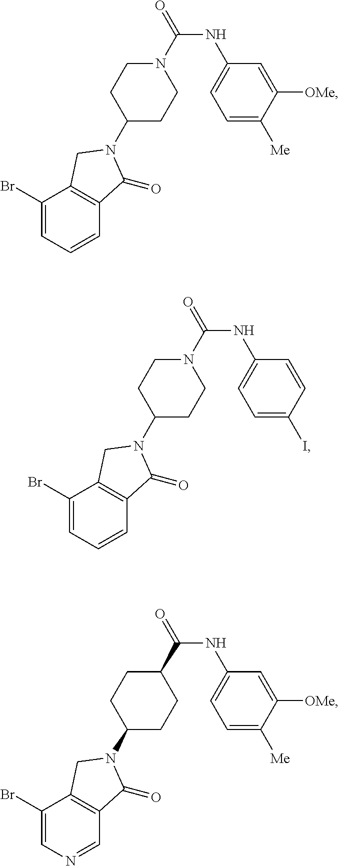 [SMILES: COc1cc(NC(=O)N2CCC(N3Cc4c(Br)cccc4C3=O)CC2)ccc1C ; O=C(Nc1ccc(I)cc1)N1CCC(N2Cc3c(Br)cccc3C2=O)CC1 ; COc1cc(NC(=O)[C@H]2CC[C@@H](N3Cc4c(Br)cncc4C3=O)CC2)ccc1C]

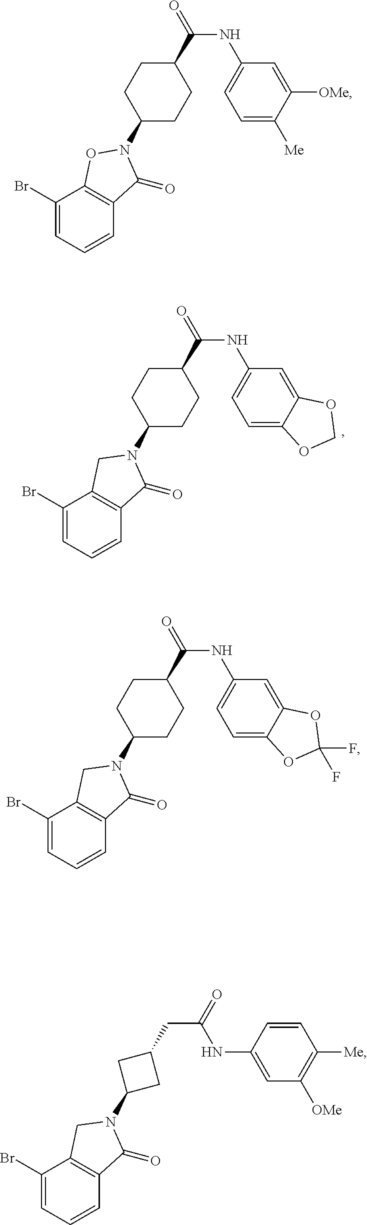 [SMILES: COc1cc(NC(=O)[C@H]2CC[C@@H](n3oc4c(Br)cccc4c3=O)CC2)ccc1C ; O=C1c2cccc(Br)c2CN1[C@H]1CC[C@@H](C(=O)Nc2ccc3c(c2)OCO3)CC1 ; O=C1c2cccc(Br)c2CN1[C@H]1CC[C@@H](C(=O)Nc2ccc3c(c2)OC(F)(F)O3)CC1 ; COc1cc(NC(=O)C[C@H]2C[C@H](N3Cc4c(Br)cccc4C3=O)C2)ccc1C]

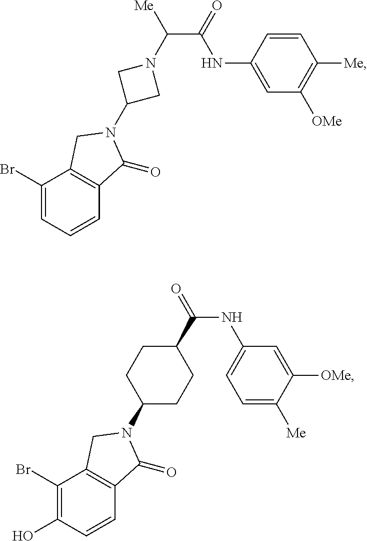 [SMILES: COc1cc(NC(=O)C(C)N2CC(N3Cc4c(Br)cccc4C3=O)C2)ccc1C ; COc1cc(NC(=O)[C@H]2CC[C@@H](N3Cc4c(ccc(O)c4Br)C3=O)CC2)ccc1C]

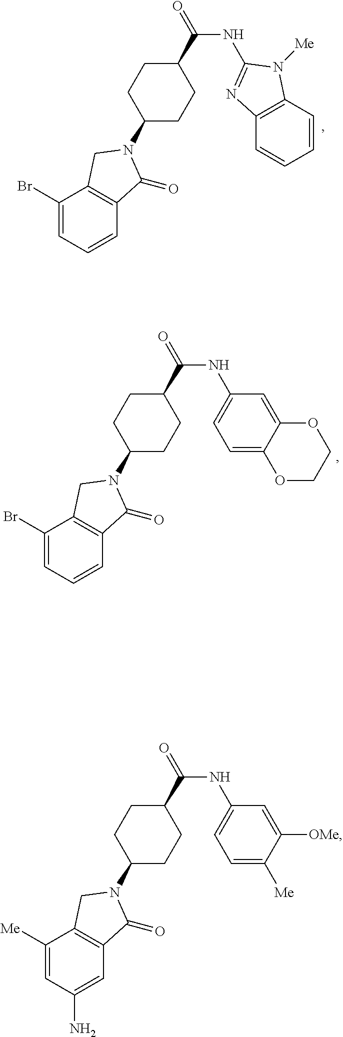 [SMILES: Cn1c(NC(=O)[C@H]2CC[C@@H](N3Cc4c(Br)cccc4C3=O)CC2)nc2ccccc21 ; O=C1c2cccc(Br)c2CN1[C@H]1CC[C@@H](C(=O)Nc2ccc3c(c2)OCCO3)CC1 ; COc1cc(NC(=O)[C@H]2CC[C@@H](N3Cc4c(C)cc(N)cc4C3=O)CC2)ccc1C]

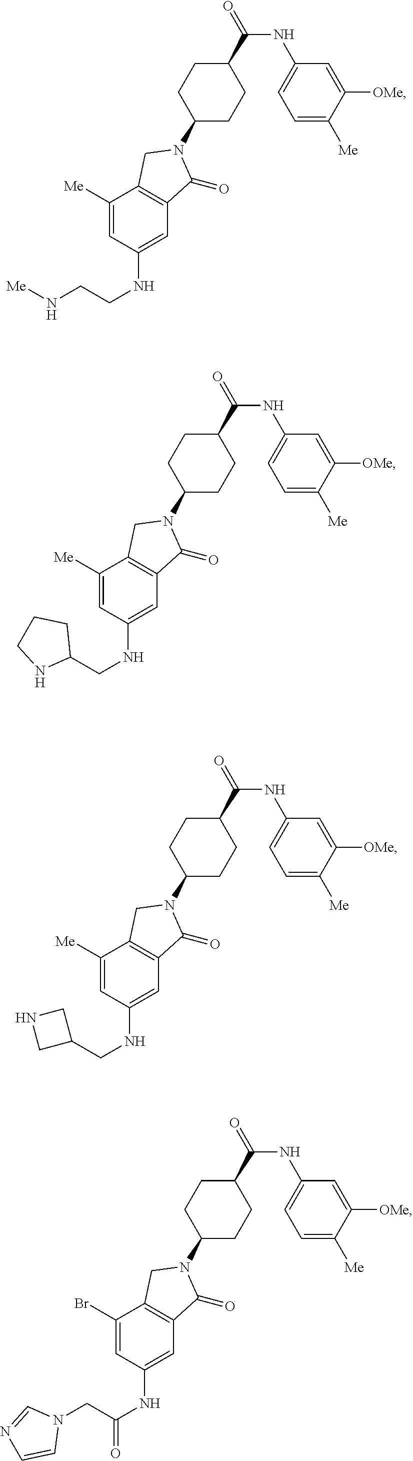 [SMILES: CNCCNc1cc(C)c2c(c1)C(=O)N([C@H]1CC[C@@H](C(=O)Nc3ccc(C)c(OC)c3)CC1)C2 ; COc1cc(NC(=O)[C@H]2CC[C@@H](N3Cc4c(C)cc(NCC5CCCN5)cc4C3=O)CC2)ccc1C ; COc1cc(NC(=O)[C@H]2CC[C@@H](N3Cc4c(C)cc(NCC5CNC5)cc4C3=O)CC2)ccc1C ; COc1cc(NC(=O)[C@H]2CC[C@@H](N3Cc4c(Br)cc(NC(=O)Cn5ccnc5)cc4C3=O)CC2)ccc1C]

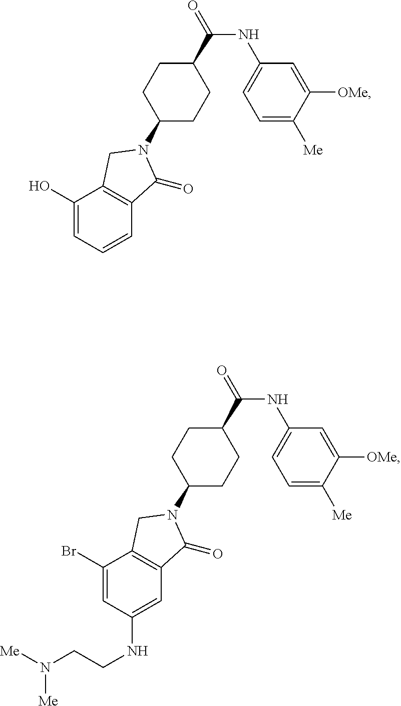 [SMILES: COc1cc(NC(=O)[C@H]2CC[C@@H](N3Cc4c(O)cccc4C3=O)CC2)ccc1C ; COc1cc(NC(=O)[C@H]2CC[C@@H](N3Cc4c(Br)cc(NCCN(C)C)cc4C3=O)CC2)ccc1C]

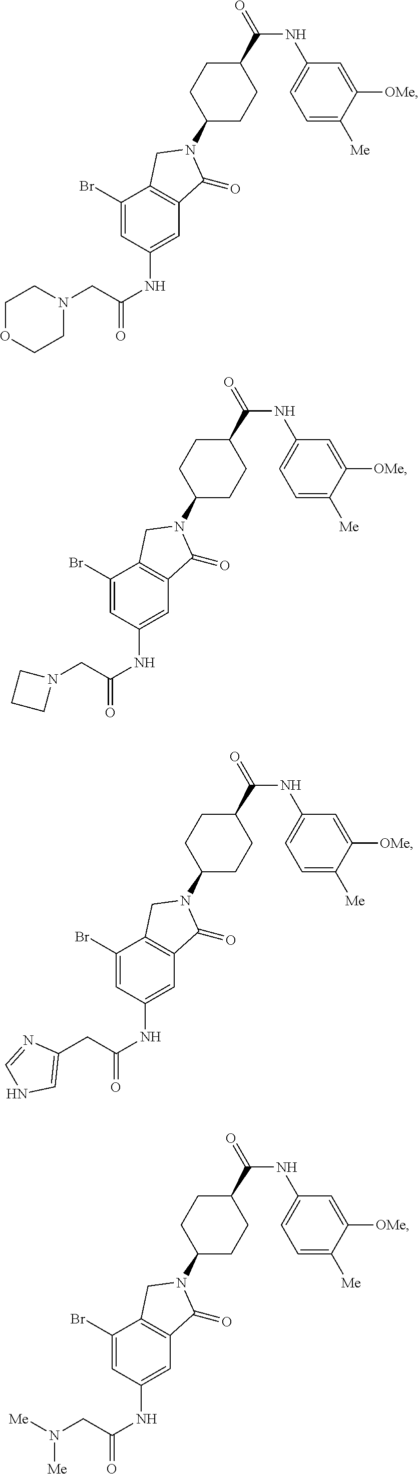 [SMILES: COc1cc(NC(=O)[C@H]2CC[C@@H](N3Cc4c(Br)cc(NC(=O)CN5CCOCC5)cc4C3=O)CC2)ccc1C ; COc1cc(NC(=O)[C@H]2CC[C@@H](N3Cc4c(Br)cc(NC(=O)CN5CCC5)cc4C3=O)CC2)ccc1C ; COc1cc(NC(=O)[C@H]2CC[C@@H](N3Cc4c(Br)cc(NC(=O)Cc5c[nH]cn5)cc4C3=O)CC2)ccc1C ; COc1cc(NC(=O)[C@H]2CC[C@@H](N3Cc4c(Br)cc(NC(=O)CN(C)C)cc4C3=O)CC2)ccc1C]

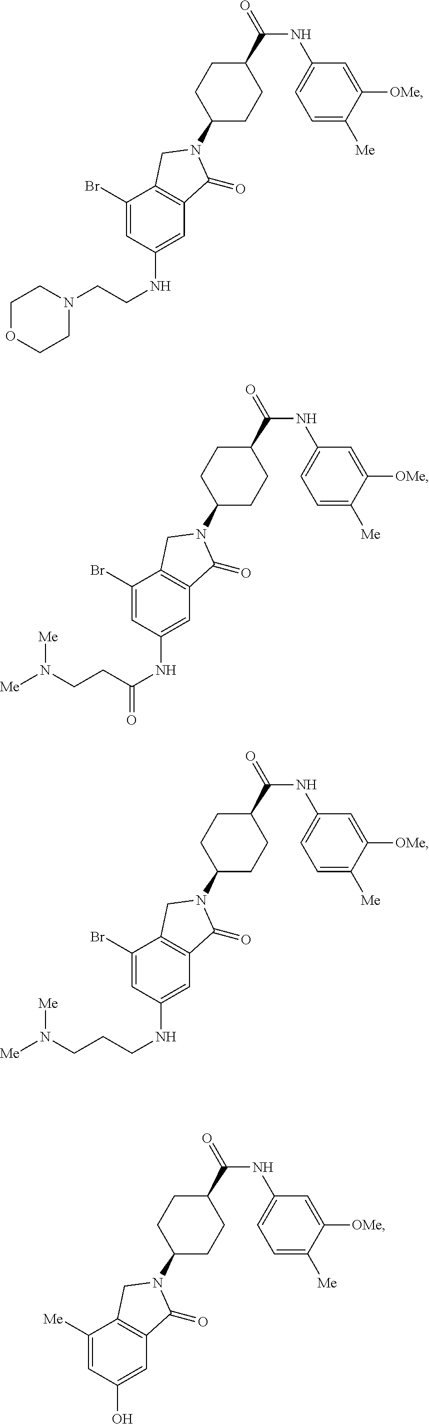 [SMILES: COc1cc(NC(=O)[C@H]2CC[C@@H](N3Cc4c(Br)cc(NCCN5CCOCC5)cc4C3=O)CC2)ccc1C ; COc1cc(NC(=O)[C@H]2CC[C@@H](N3Cc4c(Br)cc(NC(=O)CCN(C)C)cc4C3=O)CC2)ccc1C ; COc1cc(NC(=O)[C@H]2CC[C@@H](N3Cc4c(Br)cc(NCCCN(C)C)cc4C3=O)CC2)ccc1C ; COc1cc(NC(=O)[C@H]2CC[C@@H](N3Cc4c(C)cc(O)cc4C3=O)CC2)ccc1C]

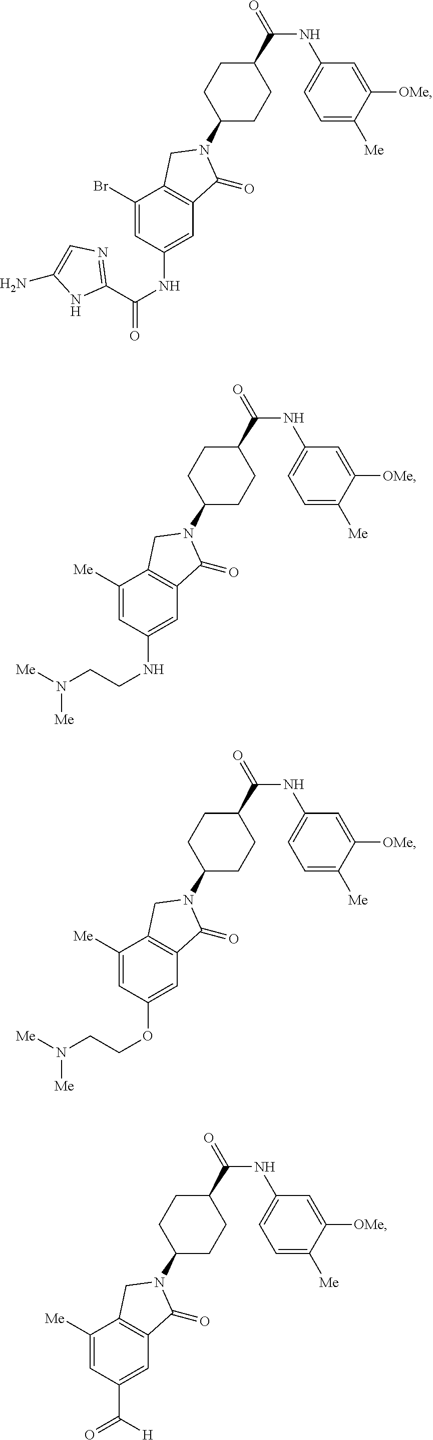 [SMILES: COc1cc(NC(=O)[C@H]2CC[C@@H](N3Cc4c(Br)cc(NC(=O)c5ncc(N)[nH]5)cc4C3=O)CC2)ccc1C ; COc1cc(NC(=O)[C@H]2CC[C@@H](N3Cc4c(C)cc(NCCN(C)C)cc4C3=O)CC2)ccc1C ; COc1cc(NC(=O)[C@H]2CC[C@@H](N3Cc4c(C)cc(OCCN(C)C)cc4C3=O)CC2)ccc1C ; COc1cc(NC(=O)[C@H]2CC[C@@H](N3Cc4c(C)cc(C=O)cc4C3=O)CC2)ccc1C]

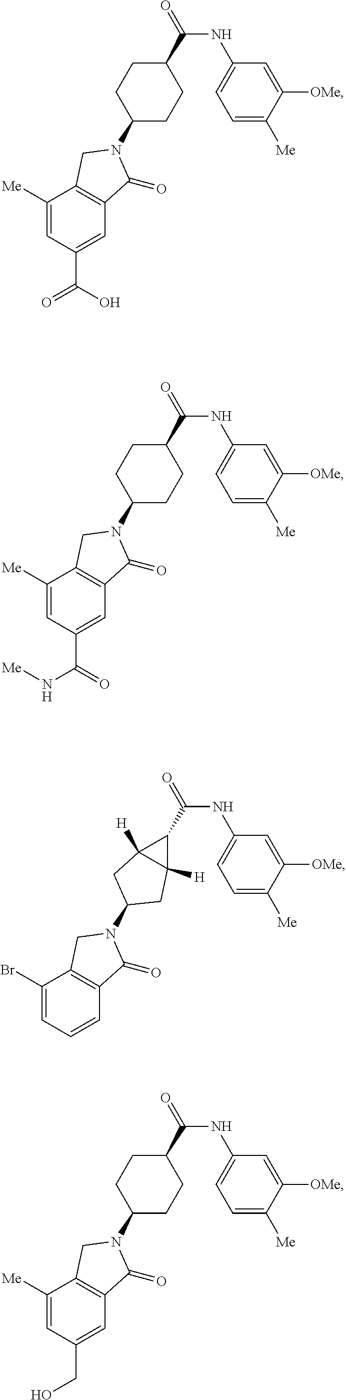 [SMILES: COc1cc(NC(=O)[C@H]2CC[C@@H](N3Cc4c(C)cc(C(=O)O)cc4C3=O)CC2)ccc1C ; CNC(=O)c1cc(C)c2c(c1)C(=O)N([C@H]1CC[C@@H](C(=O)Nc3ccc(C)c(OC)c3)CC1)C2 ; COc1cc(NC(=O)[C@@H]2[C@@H]3C[C@@H](N4Cc5c(Br)cccc5C4=O)C[C@@H]32)ccc1C ; COc1cc(NC(=O)[C@H]2CC[C@@H](N3Cc4c(C)cc(CO)cc4C3=O)CC2)ccc1C]

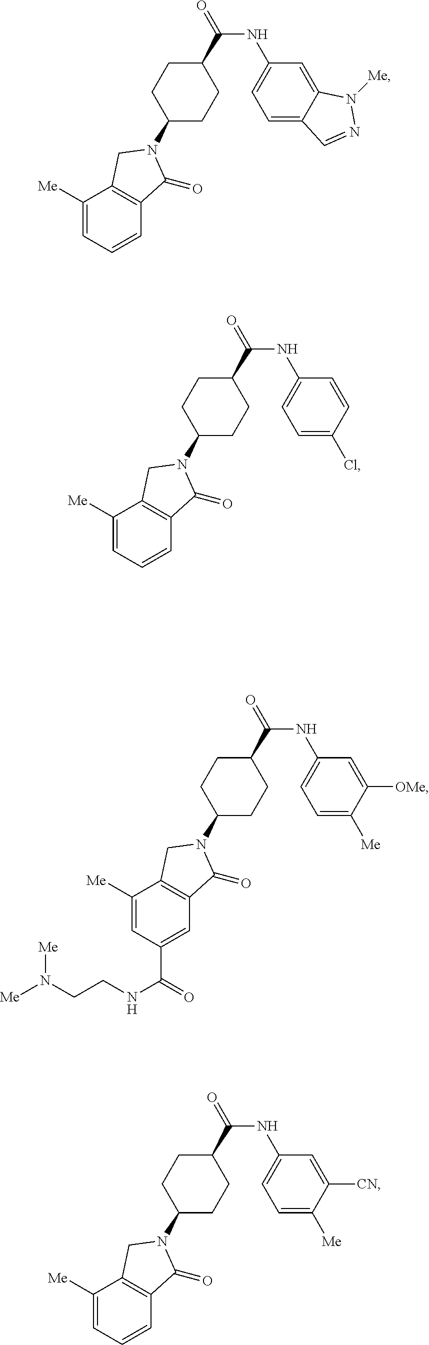 [SMILES: Cc1cccc2c1CN([C@H]1CC[C@@H](C(=O)Nc3ccc4cnn(C)c4c3)CC1)C2=O ; Cc1cccc2c1CN([C@H]1CC[C@@H](C(=O)Nc3ccc(Cl)cc3)CC1)C2=O ; COc1cc(NC(=O)[C@H]2CC[C@@H](N3Cc4c(C)cc(C(=O)NCCN(C)C)cc4C3=O)CC2)ccc1C ; Cc1ccc(NC(=O)[C@H]2CC[C@@H](N3Cc4c(C)cccc4C3=O)CC2)cc1C#N]

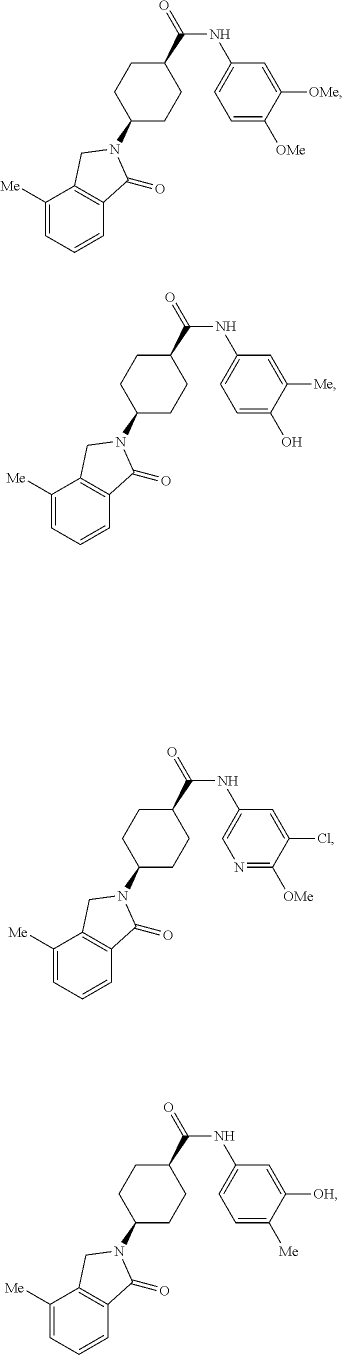 [SMILES: COc1ccc(NC(=O)[C@H]2CC[C@@H](N3Cc4c(C)cccc4C3=O)CC2)cc1OC ; Cc1cc(NC(=O)[C@H]2CC[C@@H](N3Cc4c(C)cccc4C3=O)CC2)ccc1O ; COc1ncc(NC(=O)[C@H]2CC[C@@H](N3Cc4c(C)cccc4C3=O)CC2)cc1Cl ; Cc1ccc(NC(=O)[C@H]2CC[C@@H](N3Cc4c(C)cccc4C3=O)CC2)cc1O]

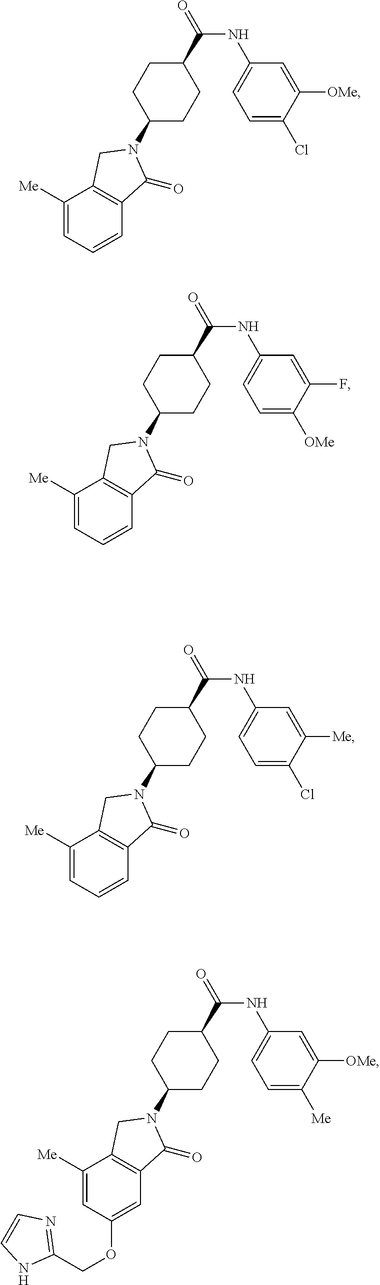 [SMILES: COc1cc(NC(=O)[C@H]2CC[C@@H](N3Cc4c(C)cccc4C3=O)CC2)ccc1Cl ; COc1ccc(NC(=O)[C@H]2CC[C@@H](N3Cc4c(C)cccc4C3=O)CC2)cc1F ; Cc1cc(NC(=O)[C@H]2CC[C@@H](N3Cc4c(C)cccc4C3=O)CC2)ccc1Cl ; COc1cc(NC(=O)[C@H]2CC[C@@H](N3Cc4c(C)cc(OCc5ncc[nH]5)cc4C3=O)CC2)ccc1C]

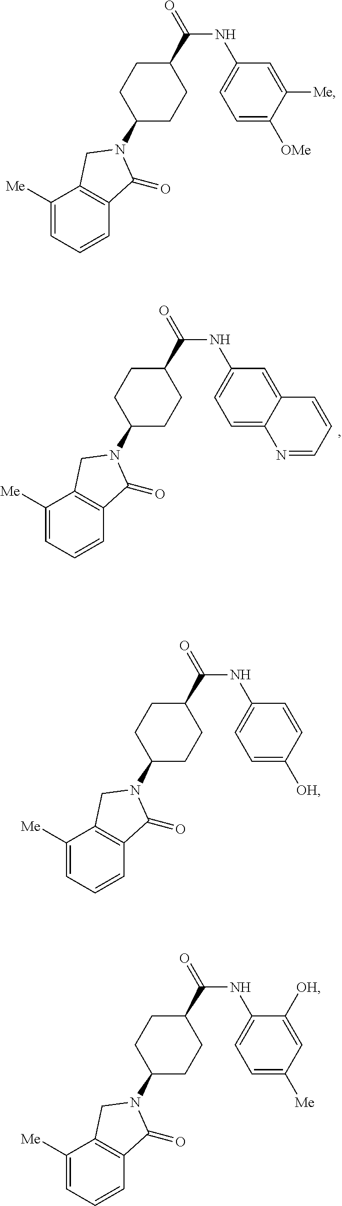 [SMILES: COc1ccc(NC(=O)[C@H]2CC[C@@H](N3Cc4c(C)cccc4C3=O)CC2)cc1C ; Cc1cccc2c1CN([C@H]1CC[C@@H](C(=O)Nc3ccc4ncccc4c3)CC1)C2=O ; Cc1cccc2c1CN([C@H]1CC[C@@H](C(=O)Nc3ccc(O)cc3)CC1)C2=O ; Cc1ccc(NC(=O)[C@H]2CC[C@@H](N3Cc4c(C)cccc4C3=O)CC2)c(O)c1]

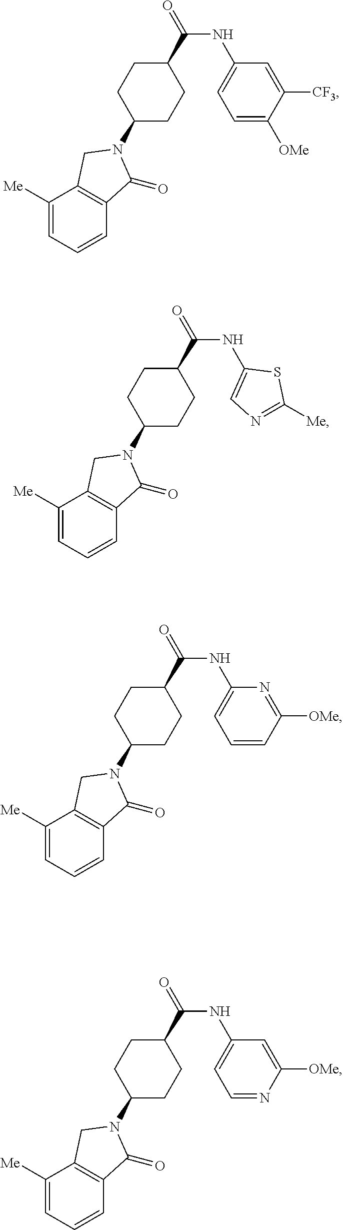 [SMILES: COc1ccc(NC(=O)[C@H]2CC[C@@H](N3Cc4c(C)cccc4C3=O)CC2)cc1C(F)(F)F ; Cc1ncc(NC(=O)[C@H]2CC[C@@H](N3Cc4c(C)cccc4C3=O)CC2)s1 ; COc1cccc(NC(=O)[C@H]2CC[C@@H](N3Cc4c(C)cccc4C3=O)CC2)n1 ; COc1cc(NC(=O)[C@H]2CC[C@@H](N3Cc4c(C)cccc4C3=O)CC2)ccn1]

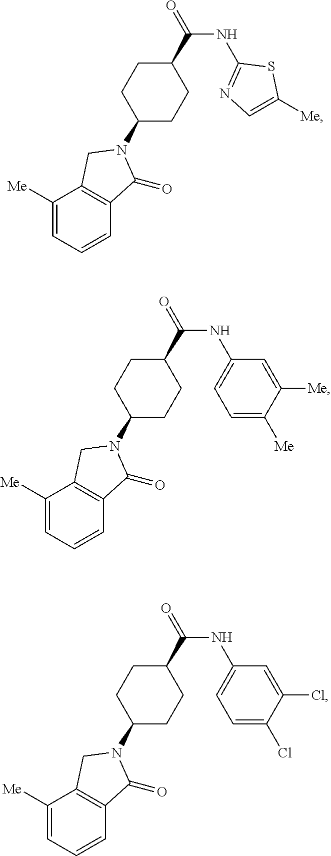 [SMILES: Cc1cnc(NC(=O)[C@H]2CC[C@@H](N3Cc4c(C)cccc4C3=O)CC2)s1 ; Cc1ccc(NC(=O)[C@H]2CC[C@@H](N3Cc4c(C)cccc4C3=O)CC2)cc1C ; Cc1cccc2c1CN([C@H]1CC[C@@H](C(=O)Nc3ccc(Cl)c(Cl)c3)CC1)C2=O]

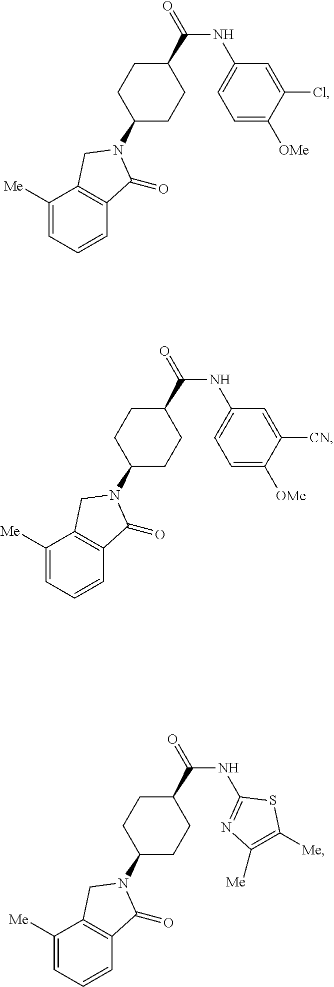 [SMILES: COc1ccc(NC(=O)[C@H]2CC[C@@H](N3Cc4c(C)cccc4C3=O)CC2)cc1Cl ; COc1ccc(NC(=O)[C@H]2CC[C@@H](N3Cc4c(C)cccc4C3=O)CC2)cc1C#N ; Cc1cccc2c1CN([C@H]1CC[C@@H](C(=O)Nc3nc(C)c(C)s3)CC1)C2=O]

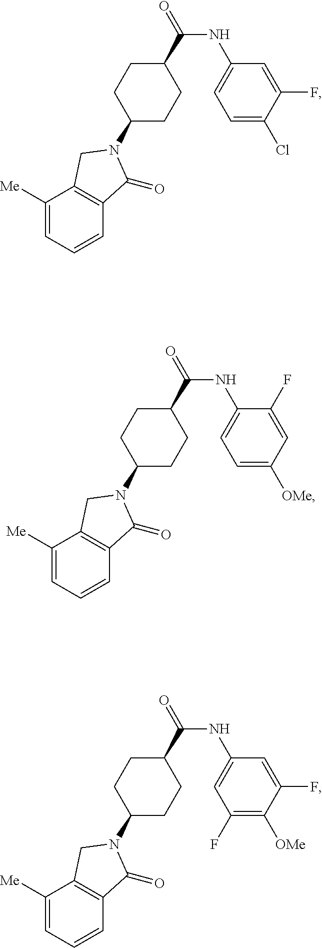 [SMILES: Cc1cccc2c1CN([C@H]1CC[C@@H](C(=O)Nc3ccc(Cl)c(F)c3)CC1)C2=O ; COc1ccc(NC(=O)[C@H]2CC[C@@H](N3Cc4c(C)cccc4C3=O)CC2)c(F)c1 ; COc1c(F)cc(NC(=O)[C@H]2CC[C@@H](N3Cc4c(C)cccc4C3=O)CC2)cc1F]

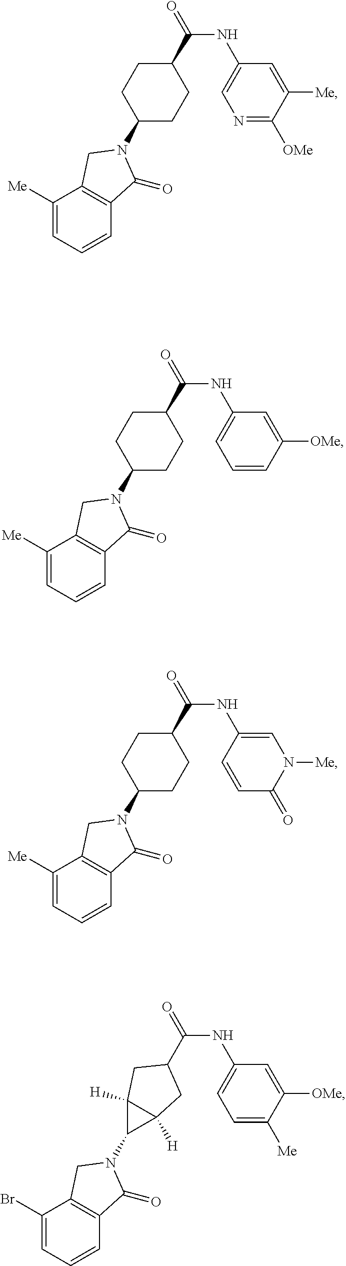 [SMILES: COc1ncc(NC(=O)[C@H]2CC[C@@H](N3Cc4c(C)cccc4C3=O)CC2)cc1C ; COc1cccc(NC(=O)[C@H]2CC[C@@H](N3Cc4c(C)cccc4C3=O)CC2)c1 ; Cc1cccc2c1CN([C@H]1CC[C@@H](C(=O)Nc3ccc(=O)n(C)c3)CC1)C2=O ; COc1cc(NC(=O)C2C[C@@H]3[C@H](C2)[C@H]3N2Cc3c(Br)cccc3C2=O)ccc1C]

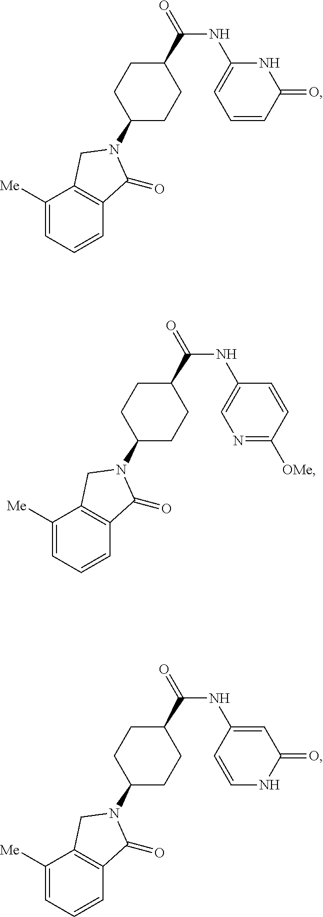 [SMILES: Cc1cccc2c1CN([C@H]1CC[C@@H](C(=O)Nc3cccc(=O)[nH]3)CC1)C2=O ; COc1ccc(NC(=O)[C@H]2CC[C@@H](N3Cc4c(C)cccc4C3=O)CC2)cn1 ; Cc1cccc2c1CN([C@H]1CC[C@@H](C(=O)Nc3cc[nH]c(=O)c3)CC1)C2=O]

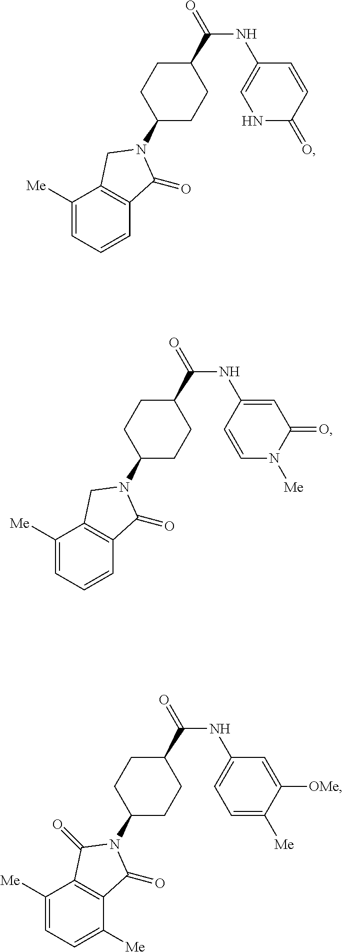 [SMILES: Cc1cccc2c1CN([C@H]1CC[C@@H](C(=O)Nc3ccc(=O)[nH]c3)CC1)C2=O ; Cc1cccc2c1CN([C@H]1CC[C@@H](C(=O)Nc3ccn(C)c(=O)c3)CC1)C2=O ; COc1cc(NC(=O)[C@H]2CC[C@@H](N3C(=O)c4c(C)ccc(C)c4C3=O)CC2)ccc1C]

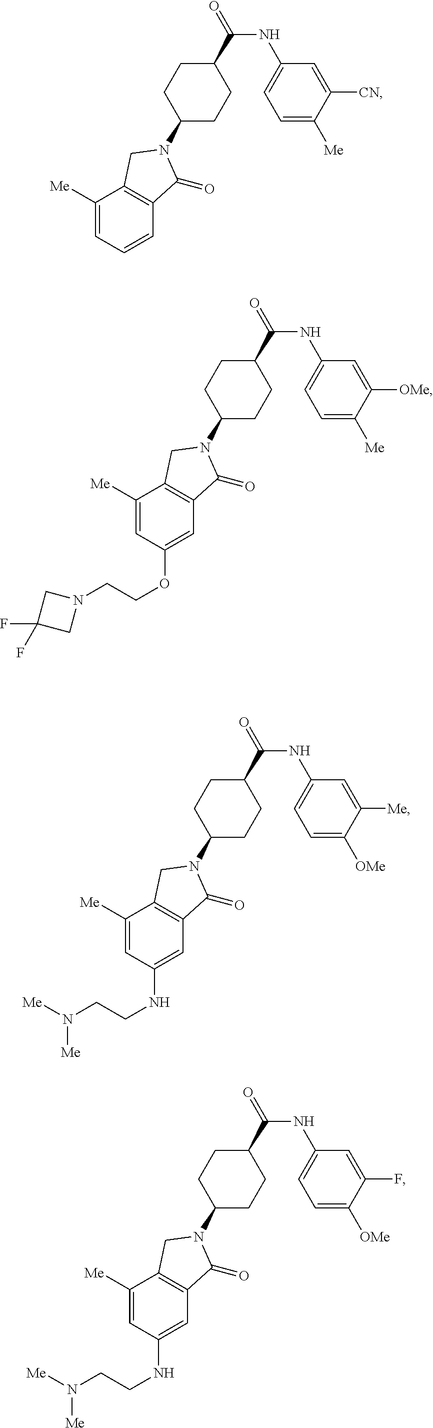 [SMILES: Cc1ccc(NC(=O)[C@H]2CC[C@@H](N3Cc4c(C)cccc4C3=O)CC2)cc1C#N ; COc1cc(NC(=O)[C@H]2CC[C@@H](N3Cc4c(C)cc(OCCN5CC(F)(F)C5)cc4C3=O)CC2)ccc1C ; COc1ccc(NC(=O)[C@H]2CC[C@@H](N3Cc4c(C)cc(NCCN(C)C)cc4C3=O)CC2)cc1C ; COc1ccc(NC(=O)[C@H]2CC[C@@H](N3Cc4c(C)cc(NCCN(C)C)cc4C3=O)CC2)cc1F]

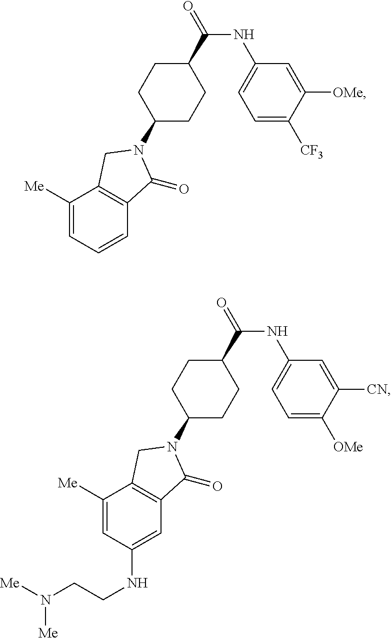 [SMILES: COc1cc(NC(=O)[C@H]2CC[C@@H](N3Cc4c(C)cccc4C3=O)CC2)ccc1C(F)(F)F ; COc1ccc(NC(=O)[C@H]2CC[C@@H](N3Cc4c(C)cc(NCCN(C)C)cc4C3=O)CC2)cc1C#N]

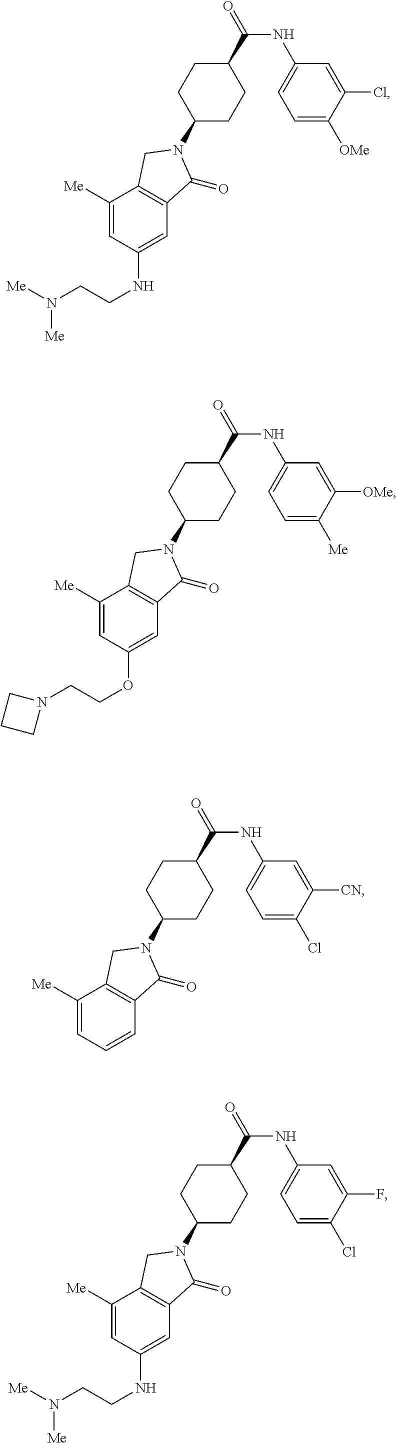 [SMILES: COc1ccc(NC(=O)[C@H]2CC[C@@H](N3Cc4c(C)cc(NCCN(C)C)cc4C3=O)CC2)cc1Cl ; COc1cc(NC(=O)[C@H]2CC[C@@H](N3Cc4c(C)cc(OCCN5CCC5)cc4C3=O)CC2)ccc1C ; Cc1cccc2c1CN([C@H]1CC[C@@H](C(=O)Nc3ccc(Cl)c(C#N)c3)CC1)C2=O ; Cc1cc(NCCN(C)C)cc2c1CN([C@H]1CC[C@@H](C(=O)Nc3ccc(Cl)c(F)c3)CC1)C2=O]

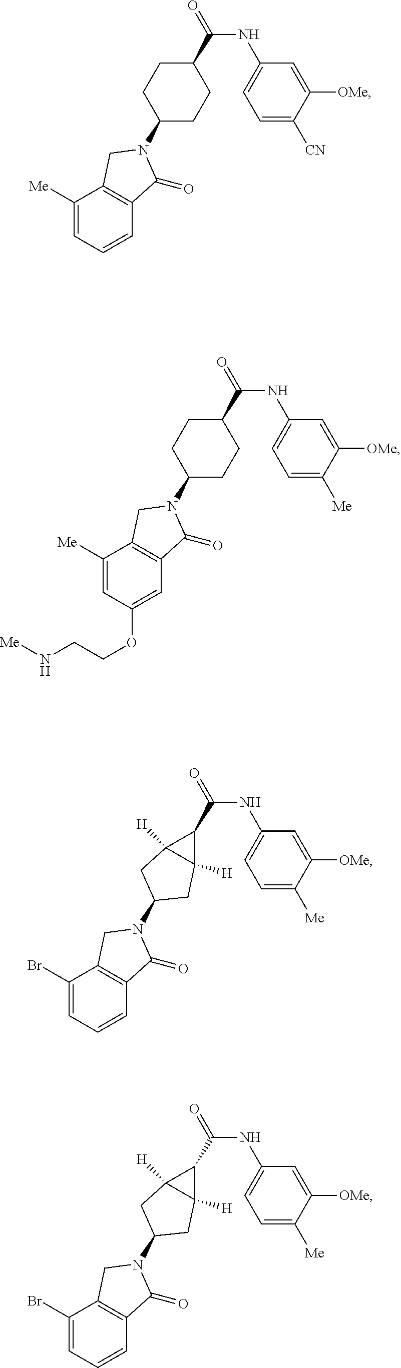 [SMILES: COc1cc(NC(=O)[C@H]2CC[C@@H](N3Cc4c(C)cccc4C3=O)CC2)ccc1C#N ; CNCCOc1cc(C)c2c(c1)C(=O)N([C@H]1CC[C@@H](C(=O)Nc3ccc(C)c(OC)c3)CC1)C2 ; COc1cc(NC(=O)[C@@H]2[C@@H]3C[C@H](N4Cc5c(Br)cccc5C4=O)C[C@@H]32)ccc1C ; COc1cc(NC(=O)[C@H]2[C@@H]3C[C@H](N4Cc5c(Br)cccc5C4=O)C[C@@H]32)ccc1C]

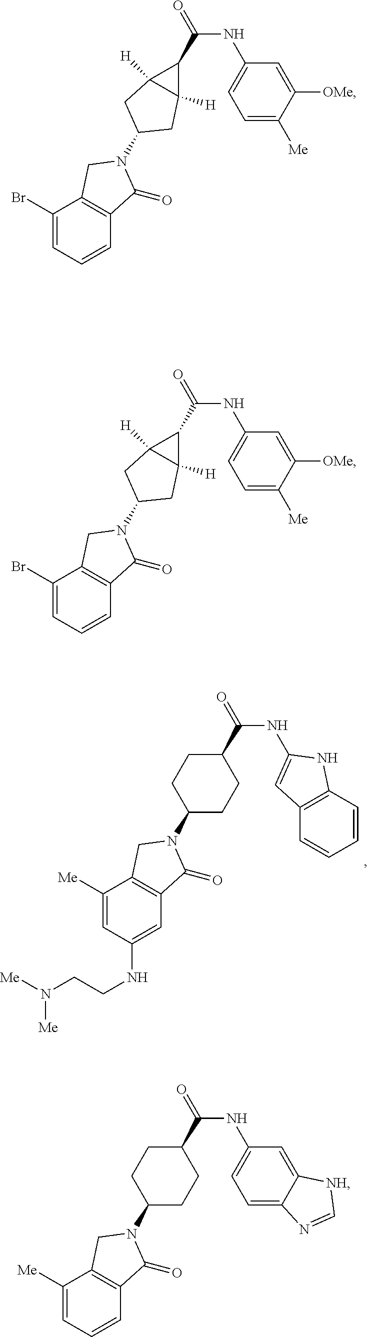 [SMILES: COc1cc(NC(=O)[C@@H]2[C@@H]3C[C@@H](N4Cc5c(Br)cccc5C4=O)C[C@@H]32)ccc1C ; COc1cc(NC(=O)[C@H]2[C@@H]3C[C@@H](N4Cc5c(Br)cccc5C4=O)C[C@@H]32)ccc1C ; Cc1cc(NCCN(C)C)cc2c1CN([C@H]1CC[C@@H](C(=O)Nc3cc4ccccc4[nH]3)CC1)C2=O ; Cc1cccc2c1CN([C@H]1CC[C@@H](C(=O)Nc3ccc4nc[nH]c4c3)CC1)C2=O]

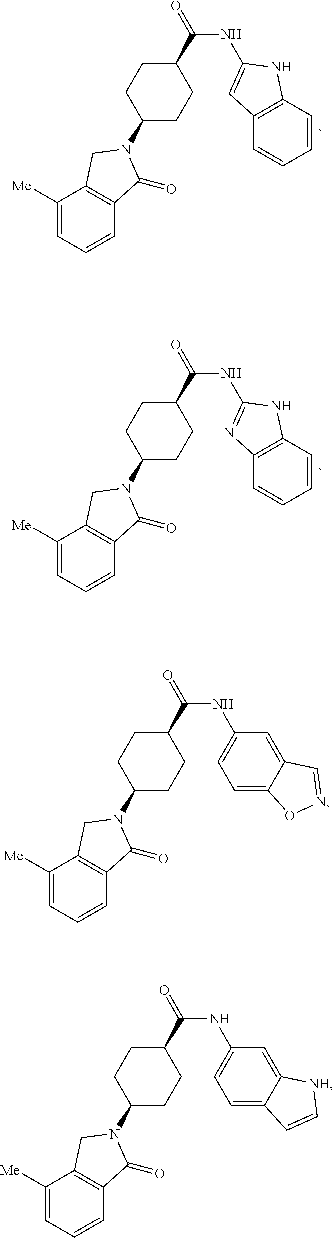 [SMILES: Cc1cccc2c1CN([C@H]1CC[C@@H](C(=O)Nc3cc4ccccc4[nH]3)CC1)C2=O ; Cc1cccc2c1CN([C@H]1CC[C@@H](C(=O)Nc3nc4ccccc4[nH]3)CC1)C2=O ; Cc1cccc2c1CN([C@H]1CC[C@@H](C(=O)Nc3ccc4oncc4c3)CC1)C2=O ; Cc1cccc2c1CN([C@H]1CC[C@@H](C(=O)Nc3ccc4cc[nH]c4c3)CC1)C2=O]

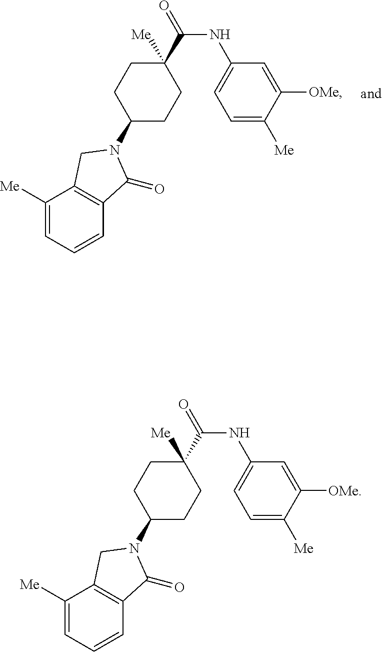 [SMILES: COc1cc(NC(=O)[C@]2(C)CC[C@H](N3Cc4c(C)cccc4C3=O)CC2)ccc1C ; COc1cc(NC(=O)[C@]2(C)CC[C@@H](N3Cc4c(C)cccc4C3=O)CC2)ccc1C]

In further embodiments, the compounds, stereoisomers, or pharmaceutically acceptable salts thereof described herein are selected from the group consisting of

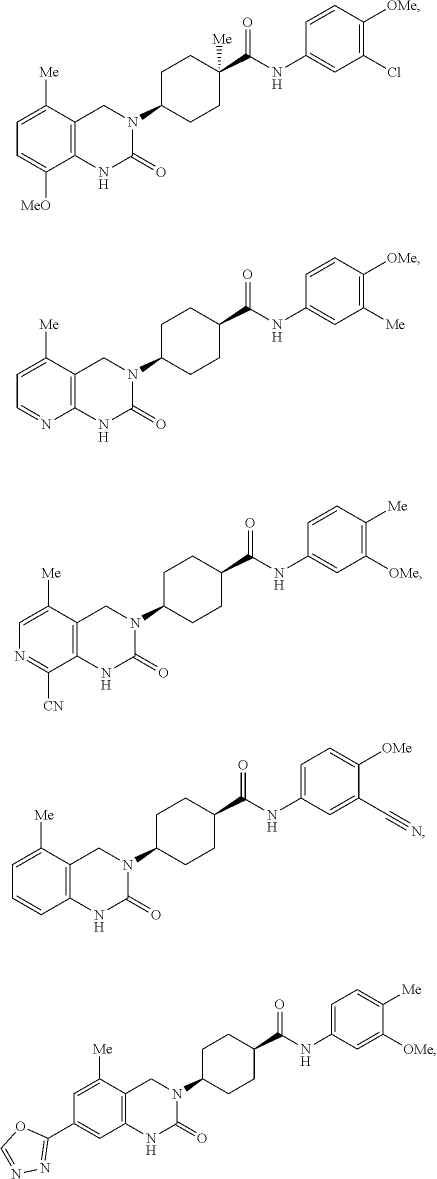 [SMILES: COc1ccc(NC(=O)[C@]2(C)CC[C@H](N3Cc4c(C)ccc(OC)c4NC3=O)CC2)cc1Cl ; COc1ccc(NC(=O)[C@H]2CC[C@@H](N3Cc4c(C)ccnc4NC3=O)CC2)cc1C ; COc1cc(NC(=O)[C@H]2CC[C@@H](N3Cc4c(C)cnc(C#N)c4NC3=O)CC2)ccc1C ; COc1ccc(NC(=O)[C@H]2CC[C@@H](N3Cc4c(C)cccc4NC3=O)CC2)cc1C#N ; COc1cc(NC(=O)[C@H]2CC[C@@H](N3Cc4c(C)cc(-c5nnco5)cc4NC3=O)CC2)ccc1C]

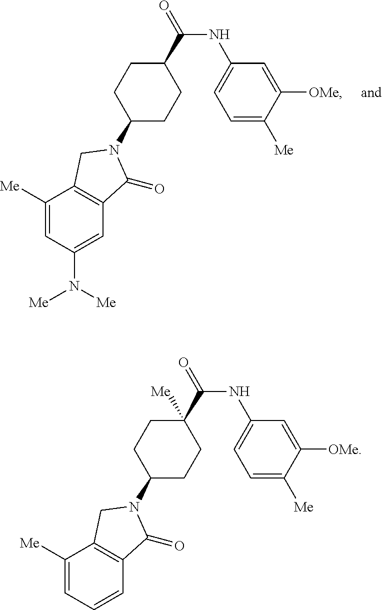 [SMILES: COc1cc(NC(=O)[C@H]2CC[C@@H](N3Cc4c(C)cc(N(C)C)cc4C3=O)CC2)ccc1C ; COc1cc(NC(=O)[C@]2(C)CC[C@H](N3Cc4c(C)cccc4C3=O)CC2)ccc1C]

In further embodiments, the compounds, stereoisomers, or pharmaceutically acceptable salts thereof described herein is

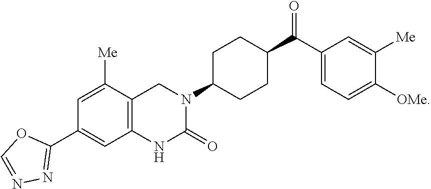 [SMILES: COc1ccc(C(=O)[C@H]2CC[C@@H](N3Cc4c(C)cc(-c5nnco5)cc4NC3=O)CC2)cc1C]

In further embodiments, the compounds, stereoisomers, or pharmaceutically acceptable salts thereof described herein is

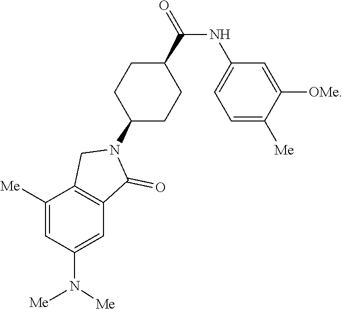 [SMILES: COc1cc(NC(=O)[C@H]2CC[C@@H](N3Cc4c(C)cc(N(C)C)cc4C3=O)CC2)ccc1C]

In some embodiments, the compounds, stereoisomers, or pharmaceutically acceptable salts thereof described herein are suitable for use in medicine. In further aspects, the compounds, stereoisomers, or pharmaceutically acceptable salts thereof described herein are for use in the treatment of a disorder that is responsive to inhibition of OGG1.

In some embodiments, the compounds, stereoisomers, or pharmaceutically acceptable salts thereof described herein are for use in the manufacture of a medicament for the treatment of a disorder that is responsive to inhibition of OGG1. In some aspects, the disorder is selected from the group consisting of cancer, cardiovascular disorder, neurological disorder, inflammatory disorder, autoimmune disorder, and infectious disease, as further described herein.

### Pharmaceutical Compositions

The compounds of Formulae I, II, IIIa, IIIb, IVa, and IVb, and stereoisomers and pharmaceutically acceptable salts thereof may be used on their own but will generally be administered in the form of a pharmaceutical composition in which the disclosed compound and stereoisomers and pharmaceutically acceptable salts thereof are in association with a pharmaceutically acceptable adjuvant, diluent, or carrier. Conventional procedures for the selection and preparation of suitable pharmaceutical formulations are described in, for example, “Pharmaceuticals—The Science of Dosage Form Designs”, M. E. Aulton, Churchill Livingstone, 1988, which is hereby incorporated by reference in its entirety.

The present disclosure also provides a pharmaceutical composition comprising a compound of Formulae I, II, IIIa, IIIb, IVa, or IVb, and stereoisomers and pharmaceutically acceptable salts thereof, and a pharmaceutically acceptable carrier.

The present disclosure also provides a compound of Formulae I, II, IIIa, IIIb, IVa, or IVb, and stereoisomers and pharmaceutically acceptable salts thereof, for use in medicine.

The present disclosure also provides a pharmaceutical composition comprising a compound of Formulae I, II, IIIa, IIIb, IVa, or IVb, and stereoisomers and pharmaceutically acceptable salts thereof, in association with a pharmaceutically acceptable adjuvant, diluent or carrier.

The present disclosure further provides a process for the preparation of a pharmaceutical composition of the present disclosure which comprises mixing a compound of Formulae I, II, IIIa, IIIb, IVa, or IVb, and stereoisomers and pharmaceutically acceptable salts thereof with a pharmaceutically acceptable adjuvant, diluent or carrier.

Depending on the mode of administration, the pharmaceutical composition will comprise from about 0.05 to about 99 wt-% (percent by weight), more particularly from about 0.05 to about 80 wt-%, still more particularly from about 0.10 to about 70 wt-%, and even more particularly from about 0.10 to about 50 wt-%, of active ingredient, all percentages by weight being based on the total composition.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the present disclosure (including stereoisomers and pharmaceutically acceptable salts thereof) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the present disclosure may also be administered by means of a dry powder inhaler. The inhaler may be a single or a multi dose inhaler, and may be a breath actuated dry powder inhaler.

One possibility is to mix the finely divided compound of the present disclosure with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively, the finely divided compound may be coated by another substance. The powder mixture may also be dispensed into hard gelatin capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

Another possibility is to process the compound as an amorphous dispersion in a polymer matrix such as hydroxypropyl methylcellulose (HPMC) or hydroxypropyl methylcellulose acetate succinate (HPMCAS). As the name suggests, spray-dried dispersions (SDDs) are obtained by dissolving drug and polymer in an organic solvent, atomizing the resulting solution into droplets, and evaporation to dried solid particles. SDDs are usually amenable for use a variety of final oral dosage forms, including capsules and tablets.

For oral administration the compound of the present disclosure may be admixed with an adjuvant or a carrier, for example, lactose, saccharose, sorbitol, mannitol; a starch, for example, potato starch, corn starch or amylopectin; a cellulose derivative; a binder, for example, gelatin or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatin, talcum and titanium dioxide. Alternatively, the tablet may be coated with a suitable polymer dissolved in a readily volatile organic solvent.

For the preparation of soft gelatin capsules, the compound of the present disclosure may be admixed with, for example, a vegetable oil or polyethylene glycol. Hard gelatin capsules may contain granules of the compound using the above-mentioned excipients for tablets. Further, liquid or semisolid formulations of the compound of the present disclosure may be filled into hard gelatin capsules.

Liquid preparations for oral application may be in the form of syrups or suspensions, for example, solutions containing the compound of the present disclosure, the balance being sugar and a mixture of ethanol, water, glycerol and propylene glycol. Optionally, such liquid preparations may contain coloring agents, flavoring agents, saccharine and/or carboxymethylcellulose as a thickening agent or other excipients known to those skilled in art.

### Methods of Treatment

The terms “treat,” “treating,” or “treatment”, as used herein, refer to any indicia of success in the amelioration of a disorder (such as injury, disease pathology, or condition), including any objective or subjective parameter such as abatement; remission; diminishing of symptoms or making the disorder more tolerable to the subject; slowing or stopping the rate of degeneration, decline, or development; slowing the progression of disorder; making the final point of degeneration less debilitating; improving a subject's physical or mental well-being; or relieving or causing regression of the disorder. The treatment of symptoms, including the amelioration of symptoms, can be based on objective or subjective parameters, which may include the results of a physical examination, a neuropsychiatric exam, and/or a psychiatric evaluation. Certain methods and uses disclosed herein may treat cancer by, for example, causing remission of cancer, slowing the rate of growth of cancer cells, slowing the rate of spread of cancer cells, reducing metastasis, or reducing the growth of metastatic tumors, reducing the size of one or more tumors, reducing the number of one or more tumors, or any combinations thereof.

The terms “administered”, “administration”, or “administering”, as used herein, refers to either directly administering a disclosed compound (and stereoisomers and pharmaceutically acceptable salts thereof) or a composition to a subject, including an animal, in need of treatment by bringing such individual in contact with, or otherwise exposing such individual to, such compound.

As used herein, the term “subject” encompasses mammals and non-mammals. Examples of mammals include, but are not limited to, any member of the class Mammalia: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the present disclosure, the mammal is a human.

A “patient” is a mammal, e.g., a human, mouse, rat, guinea pig, dog, cat, horse, cow, pig, or non-human primate, such as a monkey, chimpanzee, baboon or rhesus. “Patient” includes both humans and animals.

“Inhibition”, as used herein, refers to reducing the activity of or complete inhibition of the target. activity may include reducing the activity of a component of a pathway to a level that is still detectable. Full inhibition may include stopping all activity of a component of a pathway (such as stopping the activity of OGG1), or reducing the activity of a component of a pathway to a level below detection. Inhibition of a component of a pathway may be measured directly or indirectly, using any methods known in the art.

The term “disorder”, as used herein, refers to and is used interchangeably with the terms disease, condition, or illness, unless otherwise indicated.

In some embodiments, the present disclosure provides compounds which are suitable for use in the treatment of one or more disorders which are linked to OGG1. “Responsive to inhibition of OGG1”, as used herein, refers to disorders expected to improve with administration of inhibiting doses of the compound. Cancer is a disorder that may be responsive to treatment by administration of the compound. Some specific cancers responsive to treatment by administration of the compound can be identified by analysis of in vitro expression levels and abnormal activity of OGG1 in cancer cell lines. Additionally, in vitro cell viability assays can be used to identify specific cancer cell lines that are responsive to treatment by administration of the compound. Cancer cell lines responsive to OGG1 inhibitor compounds in vitro are predicted to be responsive to OGG1 inhibitor compounds in vivo, for example in xenograft models for cancer growth inhibition in mice. Also, some cancers may be responsive to OGG1 inhibitors indirectly by activating the immune system of the patient rather than by causing death of the cancer cells directly. Additional methods may be used to identify OGG1-responsive cancers such tumor-specific mutations in particular genes, tumor gene expression patterns as well as biomarkers in the blood for example circulating tumor DNA, RNA or proteins.

The compounds of Formulae I, II, IIIa, IIIb, IVa, or IVb, and stereoisomers and pharmaceutically acceptable salts thereof may have activity as pharmaceuticals, as discussed herein.

The present disclosure provides a compound of Formulae I, II, IIIa, IIIb, IVa, or IVb, and stereoisomers and pharmaceutically acceptable salts thereof, or a pharmaceutical composition thereof for use in the treatment of a disorder in a subject in need thereof.

The present disclosure provides a compound of Formulae I, II, IIIa, IIIb, IVa, or IVb, and stereoisomers and pharmaceutically acceptable salts thereof, or a pharmaceutical composition thereof for the treatment of a disorder in a subject in need thereof.

The present disclosure provides a compound of Formulae I, II, IIIa, IIIb, IVa, or IVb, and stereoisomers and pharmaceutically acceptable salts thereof, or a pharmaceutical composition thereof, for use in the treatment of a disorder that is responsive to inhibition of OGG1.

The present disclosure provides a compound of Formulae I, II, IIIa, IIIb, IVa, or IVb, and stereoisomers and pharmaceutically acceptable salts thereof, or a pharmaceutical composition thereof, in the treatment of a disorder that is responsive to inhibition of OGG1.

The present disclosure provides a compound of Formulae I, II, IIIa, IIIb, IVa, or IVb, and stereoisomers and pharmaceutically acceptable salts thereof, or a pharmaceutical composition thereof, for use in the manufacture of a medicament for the treatment of a disorder that is responsive to inhibition of OGG1.

The present disclosure also provides a method of treating a disorder in a subject in need thereof, wherein the disorder is mediated by OGG1, comprising administering to the subject a compound of Formulae I, II, IIIa, IIIb, IVa, or IVb, and stereoisomers and pharmaceutically acceptable salts thereof, or a pharmaceutical composition thereof.

In some embodiments, the disorder is selected from the group consisting of cancer, cardiovascular disorder, neurological disorder, inflammatory disorder, autoimmune disorder, and infectious disease.

In some embodiments, wherein the disorder is cancer. In some embodiments, the cancer is of solid organ origin or of hematopoietic origin.

In some embodiments, the disorder is cancer, the cancer is of solid organ origin, and the solid organ is selected from the group consisting of the brain, breast, colon, endometrium, esophagus, head and neck, upper gastrointestinal tract, respiratory tract, lung, kidney, liver, lower gastrointestinal tract, small intestine, large intestine, ovary, pancreas, prostate, stomach, testes, and urinary tract. In some embodiments, the disorder is cancer, and the cancer is adenocarcinoma. In some embodiments, the disorder is non-small cell lung cancer. In some embodiments, the cancer is squamous cell carcinoma of the lung (SCCL).

In some embodiments, the disorder is cancer, and the cancer is leukemia or lymphoma. In some embodiments, the leukemia is acute lymphocytic leukemia (ALL), acute myelogenous leukemia (AML), chronic lymphocytic leukemia (CLL), or chronic myelogenous leukemia (CML). In some embodiments, the lymphoma is Hodgkin's lymphoma, non-Hodgkin's lymphoma, multiple myeloma, B-cell lymphoma, diffuse large B-cell lymphoma (DLBCL), chronic lymphocyteic lymphoma (CLL), T-cell lymphoma, hairy cell lymphoma, or Burkett's lymphoma.

In some embodiments, the disorder is cancer, and the cancer is selected from the group consisting of cancer of the bladder, bone cancer, cancer of the cervix, cancer of the epithelium, cancer of the gallbladder, cancer of the rectum, skin cancer, thyroid cancer, and cancer of the uterus.

In some embodiments, the disorder is an inflammatory disorder, and the inflammatory disorder is selected from the group consisting of inflammatory bowel disease (IBD), atherosclerosis, rheumatoid arthritis, fibrosis, Alzheimer's disease, and Parkinson's disease.

In some embodiments, a compound of Formulae I, II, IIIa, IIIb, IVa, or IVb, and stereoisomers and pharmaceutically acceptable salts thereof, is administered to a subject in need thereof to inhibit a component of the OGG1 pathway. In certain embodiments, the compound or stereoisomer or a pharmaceutically acceptable salt thereof, is administered as a pharmaceutical composition, as described herein.

In some embodiments, inhibition of a component of the OGG1 pathway is measured directly, for example by measuring the product of a reaction catalyzed by a OGG1 pathway component.

In some embodiments, inhibition of a component of the OGG1 pathway is measured indirectly, for example by measuring the level of expression of one or more genes that are regulated by OGG1.

### EXAMPLES

### General Synthetic Procedures

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

TABLE 1
ABBREVIATIONS

| | |
|---|---|
| Ac | acetyl |
| ACN | acetonitrile |
| APTB | 5-[di(1-adamantyl)phosphino]-12,32,52-triphenyl- |
| | 12H-[1,42]bipyrazole |
| aq. | aqueous |
| atm | atmospheres |
| Boc | tert-butoxy carbonyl |
| Boc₂O | Di-t-butyl dicarbonate |
| BrettPhos | 2-(Dicyclohexylphosphino)3,6-dimethoxy-2′,4′,6′- |
| | triisopropyl-1,1′-biphenyl |
| DAST | Diethylaminosulfur trifluoride |
| DCM | DCM |
| DIPEA | N,N-Diisopropyl ethylamine |
| DMP; Dess-Martin | 1,1,1-Tris(acetyloxy)-1,1-dihydro-1,2-benziodoxol- |
| | 3-(1H)-one |
| DMA | Dimethyl adipate |
| DMF | Dimethylformamide |
| DMSO | dimethylsulfoxide |
| eq(s). | equivalent(s) |
| EDCI | 1-Ethyl-3-(3-dimethylaminopropyl)carbodiimide |
| EtOAc/EtOAc | EtOAc |
| Et | Ethyl |
| EtOH | ethanol |
| Et₃N | triethylamine |
| g | gram(s) |
| h | hour(s) |
| HATU | (Dimethylamino)-N,N-dimethyl(3H- |
| | [1,2,3]triazolo[4,5-b]pyridin-3- |
| | yloxy)methaniminium hexafluorophosphate |
| Hex | hexane |
| HOBt | 1-Hydroxybenzotriazole |
| HPLC | High pressure liquid chromatography |
| IPA | isopropanol |
| LCMS; LC-MS | liquid chromatography mass spectrometry |
| MeOH | methanol |
| mg | milligram(s) |
| min | Minute(s) |
| mL; ml | milliliter(s) |
| MS | mass spectrometry |
| mW | megawatt |
| NMe | N-methyl |
| NMP | N-Methyl-2-pyrrolidone |
| NMR | Nuclear magnetic resonance |
| Pd₂dba₃ | Tris(dibenzylideneacetone) dipalladium(0) |
| Ph | phenyl |
| r.t.; rt; RT | Room temperature |
| S.; sat. | saturated |
| TEA | triethylamine |
| TFA | trifluoroacetic acid |
| THF | tetrahydrofuran |
| TLC | Thin layer chromatography |
| Ts | Tosyl; 4-Methylphenylsulfonyl |
| X-Phos | 2-Dicyclohexylphosphino-2′,4′,6′- |
| | triisopropylbiphenyl |

Routine ¹H NMR spectra were recorded on 400, or 500 MHz spectrometers (Agilent, Oxford or Bruker) at ambient temperature. NMR solvents, d-chloroform (CDCl₃), d₆-dimethylsulfoxide (DMSO-d₆), and deuterated methanol (CD₃OD) were purchased from commercial suppliers and used without further purification. Spectra were processed using the automatic phasing and polynomial baseline correction features of the software. In cases where two adjacent peaks of equal or unequal height were observed, these two peaks may be labeled as either a multiplet or as a doublet. In the case of a doublet, a coupling constant using this software may be assigned. In any given example, one or more protons may not be observed due to obscurity by water and/or solvent peaks. Spectral data are reported as follows: chemical shift (multiplicity [singlet(s), broad singlet (bs), doublet (d), triplet (t), quartet (q), sextuplet (sex), multiplet (m), apparent (app), doublet of doublets (dd), doublet of doublet of doublets (ddd), doublet of triplets (dt)], coupling constant, integration). Chemical shifts are reported in ppm (δ), and coupling constants are reported in Hz. ¹H Resonances are referenced to solvent residual peaks for CDCl₃ (7.26 ppm), DMSO-d₆ (2.50 ppm), CD₃OD (3.30 ppm).

The HPLC-UV/MS instrumentation for product analysis consisted of a Waters Alliance 2695 with a column heater coupled with a ZQ 4000 mass spectrometer. The MS was equipped with an electrospray ionization (ESI) source and used in scan mode (100-1200 amu, source temperature: 150° C.) for both positive and negative ionization. The HPLC was equipped with a Waters photodiode array detector (PDA) 2998 (range used: 195-320 nm). The analytical method was developed on a XBridge C18 column (3.5 μm particle size, 4.6×30 mm) with a 10 mM buffer (ammonium formate pH 3.8 or ammonium bicarbonate pH 10)—A % and acetonitrile—B % as the mobile phase. A flow rate of 3 mL/min at 25° C. was set and the following gradient was used: 1) 5% B isocratic for 0.2 min, 5%-100% B in 1.8 min, 100% B for 1 min. or 2) 5% B isocratic for 0.2 min, 5%-100% B in 5.8 min, 100% B for 1 min.

In some other instances, HPLC-UV/MS instrumentation for product analysis consisted of a Agilent LCMS, Column: Waters X-Bridge C18 (50 mm×4.6 mm×3.5 μm); Column Temperature: 40° C.; Flow Rate: 1.5 mL/min; Mobile Phase: from 90% [water+10 mM NH₄HCO₃] and 5% [CH₃CN] to 0% [water+10 mM NH₄HCO₃] and 100% [CH₃CN] in 6.0 min, then under this condition for 2.0 min, finally changed to 90% [water+10 mM NH₄HCO₃] and 5% [CH₃CN] in 0.1 min and under this condition for 1 min.

### Prep-HPLC Conditions

An example of preparative HPLC condition employed to purify products is described below. Purifications were not limited to the gradient illustrated below and variations of the illustrated gradient were made according to polarity of the products obtained.

### 1.1 Chromatographic Equipment

Gilson Prep-HPLC system: GX-281 sample manager, 306 pump, 806 Manometric module, 811D DYNAMIC Mixer, UV/VIS-156
   1.2 Chromatographic condition
Column: Waters X-Bridge™ Prep C18 5 μm OBD™, 19×250 mm
Flowrate: 20 mL/min
Gradient:

TABLE 2
Chromatographic gradient conditions

| Time(min) | ACN | Water (10 mM NH₄HCO₃) |
|---|---|---|
| 0 | 10% | 90% |
| 1.0 | 10% | 90% |
| 3.95 | 45% | 55% |
| 19.10 | 60% | 40% |
| 19.35 | 95% | 5% |
| 24.50 | 95% | 5% |
| 24.80 | 10% | 90% |
| 30.80 | 10% | 90% |

Wavelength: 214 nm and 254 nm.

The following synthetic examples are provided to illustrate the present disclosure, and should not be construed as limiting thereof. In these examples, all parts and percentages are by weight, unless otherwise noted.

### Example 1

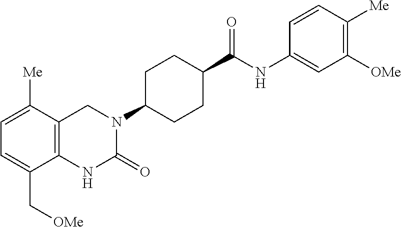 [SMILES: COCc1ccc(C)c2c1NC(=O)N([C@H]1CC[C@@H](C(=O)Nc3ccc(C)c(OC)c3)CC1)C2]

### (1s,4s)-N-(3-Methoxy-4-methylphenyl)-4-(8-(methoxymethyl)-5-methyl-2-oxo-1,2-dihydroquinazolin-3(4H)-yl)cyclohexanecarboxamide

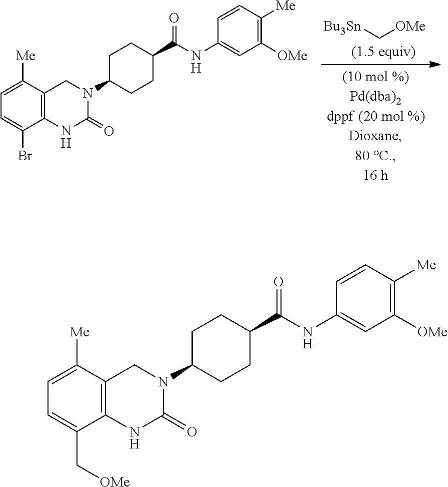

### Step 1

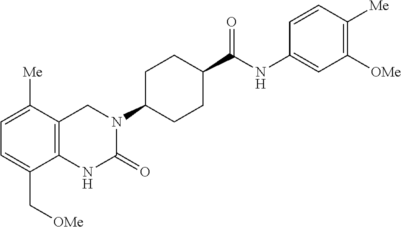 [SMILES: COCc1ccc(C)c2c1NC(=O)N([C@H]1CC[C@@H](C(=O)Nc3ccc(C)c(OC)c3)CC1)C2]

### (1s,4s)-N-(3-Methoxy-4-methylphenyl)-4-(8-(methoxymethyl)-5-methyl-2-oxo-1,2-dihydroquinazolin-3(4H)-yl)cyclohexanecarboxamide

To a microwave tube was added (1s,4s)-4-(8-bromo-5-methyl-2-oxo-1,2-dihydroquinazolin-3(4H)-yl)-N-(3-methoxy-4-methylphenyl)cyclohexanecarboxamide (50.0 mg, 84.3 μmol) and 1,4-dioxane (1.05 mL). The resulting mixture was purged with N₂ for 5 minutes then dppf (9.64 mg, 16.9 μmol), Pd (dba) 2 (4.85 mg, 8.43 μmol) was added to the mixture followed by tributyl(methoxymethyl)stannane (43.7 mg, 126 μmol). The reaction was then stirred at 80° C. for 16 h, afterwhich the reaction mixture was concentrated and directly purified. The crude residue was purified using C18 reverse phase column using MeCN:AMF (10 mM) as an eluent (5% to 65% yield). The purified product was isolated as a white powder after lyophilization (2.1 mg, 5.5% yield). LCMS ESI m/z 452.3 [M+H]⁺. ¹H NMR (400 MHz, dmso) δ 9.71 (s, 1H), 7.92 (s, 1H), 7.29 (d, J=1.7 Hz, 1H), 7.11 (dd, J=8.0, 1.8 Hz, 1H), 7.00 (dd, J=14.9, 7.9 Hz, 2H), 6.71 (d, J=7.7 Hz, 1H), 4.41 (s, 2H), 4.24 (s, 2H), 4.23-4.13 (m, 1H), 3.75 (s, 3H), 3.25 (s, 3H), 2.69-2.61 (m, 1H), 2.16 (s, 3H), 2.15-2.10 (m, 2H), 2.08 (s, 3H), 2.06-1.95 (m, 2H), 1.73-1.60 (m, 2H), 1.52-1.41 (m, 2H).

### Example 2

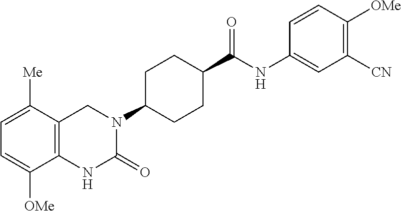 [SMILES: COc1ccc(NC(=O)[C@H]2CC[C@@H](N3Cc4c(C)ccc(OC)c4NC3=O)CC2)cc1C#N]

### (1s,4s)-N-(3-Cyano-4-methoxyphenyl)-4-(8-methoxy-5-methyl-2-oxo-1,2-dihydroquinazolin-3(4H)-yl)cyclohexanecarboxamide

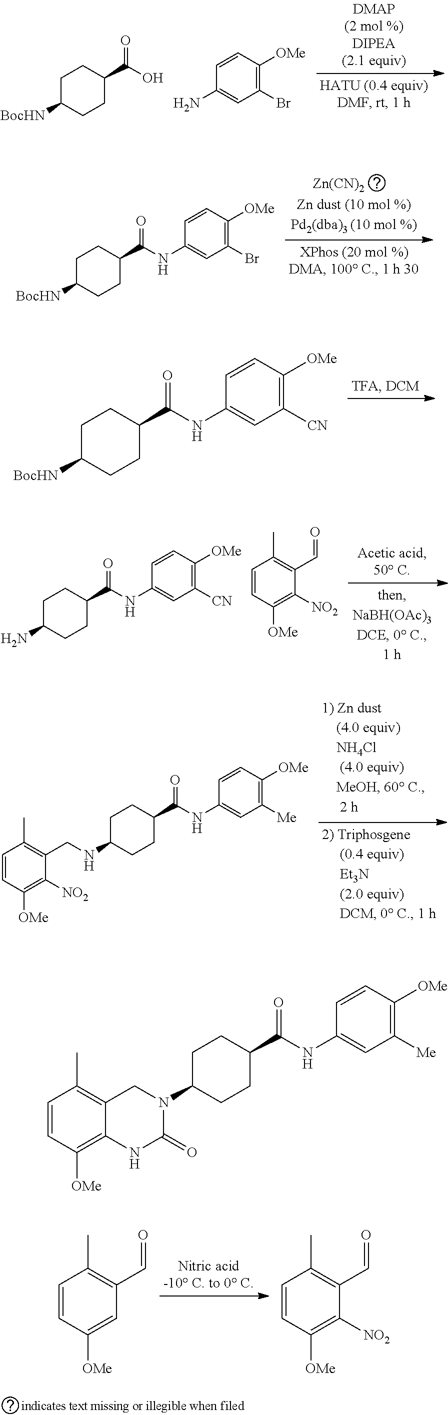

### Step 1

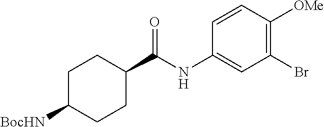 [SMILES: COc1ccc(NC(=O)[C@H]2CC[C@@H](NC(=O)OC(C)(C)C)CC2)cc1Br]

### tert-Butyl ((1s,4s)-4-((3-bromo-4-methoxyphenyl)carbamoyl)cyclohexyl)carbamate

To a solution of cis-4-(Boc-amino)cyclohexanecarboxylic acid (1.02 g, 4.11 mmol) and 3-bromo-4-methoxyaniline (892 mg, 4.19 mmol) and DMF (8.22 mL) was added N,N-diisopropylethylamine (1.52 mL, 8.63 mmol), N,N-dimethylaminopyridine (10.2 mg, 82.2 μmol) and HATU (644 mg, 1.64 mmol) at r.t. Then the reaction mixture was stirred at r.t. for 1 h. The reaction mixture was then diluted with EtOAc, washed with 1M HCl aq. and sat. NaHCO₃ and brine. The combined organic layers was dried over Na₂SO₄ and concentrated under reduced pressure. The crude product was purified using normal phase column (25 g) using Methanol: DCM (0 to 15% yield). The pure product was isolated as a white solid (0.717 g, 41% yield). LCMS ESI m/z 326.9, 328.9 [M+H]⁺.

### Step 2

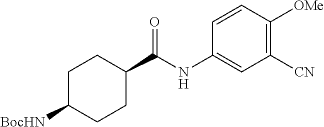 [SMILES: COc1ccc(NC(=O)[C@H]2CC[C@@H](NC(=O)OC(C)(C)C)CC2)cc1C#N]

### tert-Butyl ((1s,4s)-4-((3-Cyano-4-methoxyphenyl)carbamoyl)cyclohexyl)carbamate

A microwave vial was loaded with 2-dicyclohexylphosphino-2′,4′,6′-triisopropylbiphenyl (165 mg, 336 μmol), tris(dibenzylideneacetone)-dipalladium (0) (154 mg, 168 μmol), Zinc dust (11.0 mg, 168 μmol), Zinc cyanide (207 mg, 1.76 mmol), tert-butyl ((1s,4s)-4-((3-bromo-4-methoxyphenyl)carbamoyl)cyclohexyl)carbamate (717 mg, 1.68 mmol) and was capped and purged with nitrogen using vacuum and nitrogen backfilling (3 times). In the meantime, DMA (8.39 mL) was degassed using a stream of nitrogen bubbled in the solvent for 15 minutes. Degassed DMA was added to the reagents and the reaction mixture was heated and stirred at 100° C. for 1 h30. The reaction mixture was cooled to room temperature and filtered through Celite. The filtrate was diluted with a saturated solution of sodium bicarbonate and it was extracted 3 times with EtOAc. Organics were combined, washed with water, brine, dried with anhydrous sodium sulfate, filtered and concentrated to dryness. The crude residue was purified by flash chromatography on silica gel (50 g, 40 to 100% EtOAc in heptanes) to yield tert-Butyl ((1s,4s)-4-((3-Cyano-4-methoxyphenyl)carbamoyl)cyclohexyl)carbamate (515 mg, 82% yield) as a white solid. LCMS ESI m/z 274.0 [M+H]⁺.

### Step 3

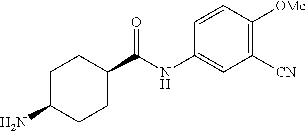 [SMILES: COc1ccc(NC(=O)[C@H]2CC[C@@H](N)CC2)cc1C#N]

### (1s,4s)-4-Amino-N-(3-cyano-4-methoxyphenyl)cyclohexanecarboxamide

To a solution of tert-Butyl ((1s,4s)-4-((3-Cyano-4-methoxyphenyl)carbamoyl)cyclohexyl)carbamate (515 mg, 1.38 mmol) in DCM (500 μL) was added trifluoroacetic acid (500 μL, 6.52 mmol) and the reaction mixture was stirred at room temperature for 3 hours. The reaction mixture was concentrated to dryness and a saturated solution of sodium bicarbonate was added to the crude residue. It was extracted 3 times with EtOAc. Organics were combined, washed with brine, dried with anhydrous sodium sulfate, filtered and concentrated to dryness to yield (1s,4s)-4-Amino-N-(3-cyano-4-methoxyphenyl)cyclohexanecarboxamide (244 mg, 65% yield) as a pale orange solid. LCMS ESI m/z 274.0 [M+H]⁺.

### Step 4

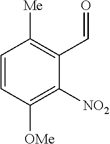 [SMILES: COc1ccc(C)c(C=O)c1[N+](=O)[O-]]

### 3-Methoxy-6-methyl-2-nitrobenzaldehyde

To a flask at 0° C. was added fuming nitric acid (5.40 mL, 106 mmol) and nitric acid (1.52 mL, 23.1 mmol). Then the internal temperature of the mixture was cooled down to −10° C. Then 5-methoxy-2-methylbenzaldehyde (2.00 g, 12.9 mmol) was added dropwise directly onto the mixture. While making sure the temperature does not go above 0° C. The mixture was kept between-10 and 0° C. for 30 min. The reaction was then quenched by adding it to ice and stirring until ice had melted. The crude material was filtered and washed with cold water. The crude material was then purified using normal phase column (25 g) from 0 to 20% EtOAc: Heptanes. The pure desired product 3-Methoxy-6-methyl-2-nitrobenzaldehyde was isolated as a yellow powder (0.582 g, 23% yield). ¹H NMR (400 MHz, CDCl₃) δ 10.21 (s, 1H), 7.35 (d, J=8.7 Hz, 1H), 7.19 (d, J=8.6 Hz, 1H), 3.91 (s, 3H), 2.60 (s, 3H).

### Step 5

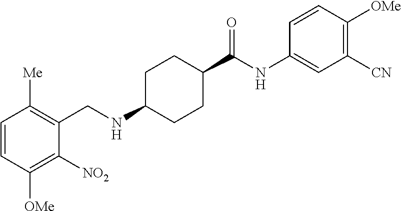 [SMILES: COc1ccc(NC(=O)[C@H]2CC[C@@H](NCc3c(C)ccc(OC)c3[N+](=O)[O-])CC2)cc1C#N]

### (1s,4s)-N-(3-Cyano-4-methoxyphenyl)-4-((3-methoxy-6-methyl-2-nitrobenzyl)amino)cyclohexanecarboxamide

To a vial with 3-methoxy-6-methyl-2-nitrobenzaldehyde (90.0 mg, 461 μmol) was added DCE (2.31 mL), (1s,4s)-4-Amino-N-(3-cyano-4-methoxyphenyl)cyclohexanecarboxamide (126 mg, 461 μmol) and acetic acid (2.65 μL, 46.1 μmol). The mixture was then heated to 50° C. overnight. The solution was cooled to 0° C. and sodium borohydride (21.9 mg, 576 μmol) was added to a cold reaction mixture and stirred for 1 h. After reaction completion, the reaction was quenched with sat. NaHCO₃, extracted with EtOAc (×3). The combined organic layers was washed with brine (×1), dried over Na₂SO₄ and concentrated under reduced pressure. The crude product was then purified using normal phase column (10 g) using MeOH: DCM as an eluent from 0 to 15%. The desired product was isolated as a white powder (46% yield, 94.2 mg). LCMS ESI m/z 453.3 [M+H]⁺.

### Step 6

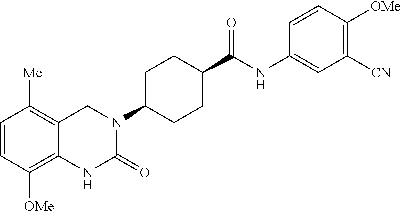 [SMILES: COc1ccc(NC(=O)[C@H]2CC[C@@H](N3Cc4c(C)ccc(OC)c4NC3=O)CC2)cc1C#N]

### (1s,4s)-N-(3-Cyano-4-methoxyphenyl)-4-(8-methoxy-5-methyl-2-oxo-1,2-dihydroquinazolin-3(4H)-yl)cyclohexanecarboxamide

To a solution of (1s,4s)-N-(3-cyano-4-methoxyphenyl)-4-((3-methoxy-6-methyl-2-nitrobenzyl)amino)cyclohexanecarboxamide (94.2 mg, 208 μmol) in MeOH (2.22 mL) was added zinc (54.5 mg, 833 μmol) and ammonium chloride (44.5 mg, 833 μmol). The reaction mixture was heated at 60° C. for 2 h. The reaction mixture was cooled, filtered through celite and evaporated in vacuo to obtain crude residue. The crude residue was dissolved in DCM (2.22 mL) and cooled down to 0° C. Then, triethylamine (58.3 μL, 416 μmol) was added to the mixture followed by triphosgene (25.2 mg, 83.3 μmol). After 1 h at 0° C., the reaction was quenched with water and concentrated under reduced pressure. The crude residue was purified using reverse phase column (C18, 10 g) using MeCN:AMF (10 mM) 0 to 70%. The product was isolated as a pale yellow solid (16% yield, 15.2 mg). LCMS ESI m/z 449.3 [M+H]⁺. ¹H NMR (400 MHz, DMSO-d₆) δ 9.95 (s, 1H), 7.99 (d, J=2.6 Hz, 1H), 7.78 (dd, J=9.2, 2.6 Hz, 1H), 7.74 (s, 1H), 7.23 (d, J=9.3 Hz, 1H), 6.78-6.65 (m, 2H), 4.21 (s, 2H), 4.21-4.15 (m, 1H), 3.88 (s, 3H), 3.74 (s, 3H), 2.72-2.58 (m, 1H), 2.18-2.06 (m, 5H), 2.06-1.90 (m, 2H), 1.75-1.60 (m, 2H), 1.52-1.41 (m, 2H).

### Example 3

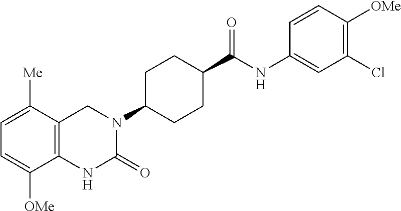 [SMILES: COc1ccc(NC(=O)[C@H]2CC[C@@H](N3Cc4c(C)ccc(OC)c4NC3=O)CC2)cc1Cl]

### (1s,4s)-N-(3-Chloro-4-methoxyphenyl)-4-(8-methoxy-5-methyl-2-oxo-1,2-dihydroquinazolin-3(4H)-yl)cyclohexanecarboxamide

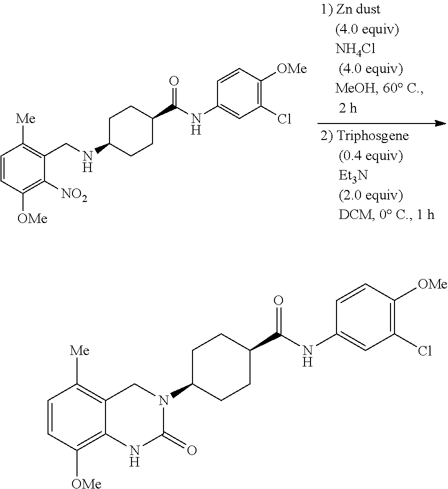 [SMILES: COc1ccc(NC(=O)[C@H]2CC[C@@H](NCc3c(C)ccc(OC)c3[N+](=O)[O-])CC2)cc1Cl ; COc1ccc(NC(=O)[C@H]2CC[C@@H](N3Cc4c(C)ccc(OC)c4NC3=O)CC2)cc1Cl]

### Step 1

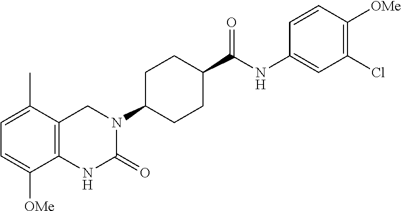 [SMILES: COc1ccc(NC(=O)[C@H]2CC[C@@H](N3Cc4c(C)ccc(OC)c4NC3=O)CC2)cc1Cl]

### (1s,4s)-N-(3-chloro-4-methoxyphenyl)-4-(8-methoxy-5-methyl-2-oxo-1,2-dihydroquinazolin-3(4H)-yl)cyclohexanecarboxamide

Following the procedure described in Example 2 and making non-critical variations as required to replace 3-bromo-4-methoxyaniline with 3-chloro-4-methoxyaniline, (1s,4s)-N-(3-chloro-4-methoxyphenyl)-4-(8-methoxy-5-methyl-2-oxo-1,2-dihydroquinazolin-3(4H)-yl)cyclohexanecarboxamide was obtained as a white solid (43 mg, 36% Yield). LCMS ESI m/z: 458.4 [M+H]⁺. ¹H NMR (400 MHz, DMSO-d₆) δ 9.79 (s, 1H), 7.81 (d, J=2.3 Hz, 1H), 7.74 (s, 1H), 7.45 (dd, J=8.9, 2.3 Hz, 1H), 7.10 (d, J=8.9 Hz, 1H), 6.68 (d, J=8.4 Hz, 1H), 6.65 (d, J=8.4 Hz, 1H), 4.25-4.15 (m, 3H), 3.81 (s, 3H), 3.74 (s, 3H), 2.64 (s, 1H), 2.19-2.06 (m, 5H), 2.06-1.92 (m, 2H), 1.73-1.61 (m, 2H), 1.52-1.40 (m, 2H).

### Example 4

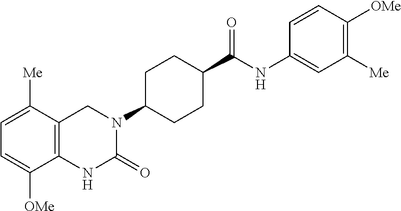 [SMILES: COc1ccc(NC(=O)[C@H]2CC[C@@H](N3Cc4c(C)ccc(OC)c4NC3=O)CC2)cc1C]

### (1s,4s)-N-(3-Methyl-4-methoxyphenyl)-4-(8-methoxy-5-methyl-2-oxo-1,2-dihydroquinazolin-3(4H)-yl)cyclohexanecarboxamide

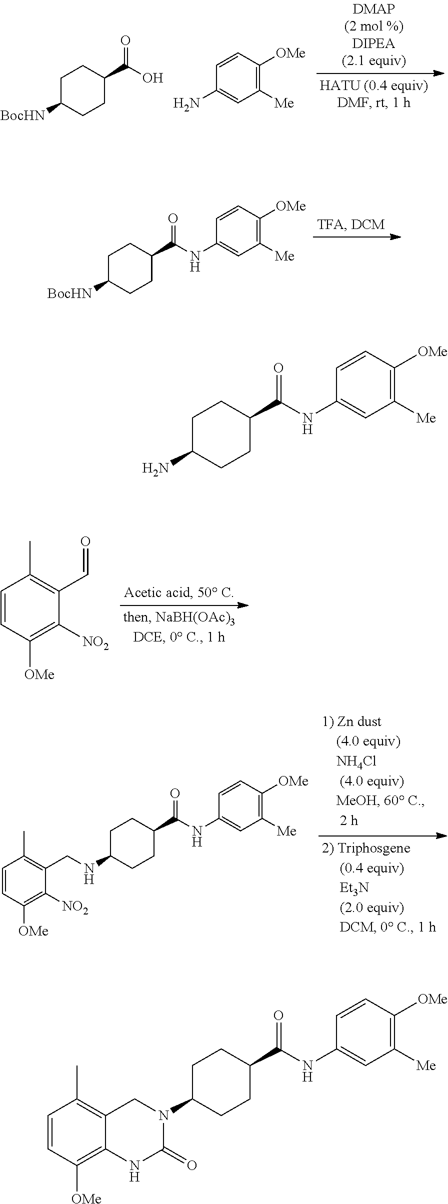

### Step 1-5

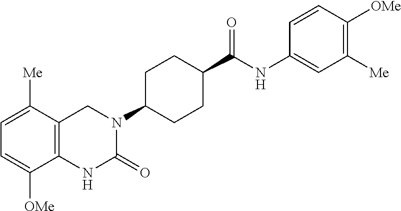 [SMILES: COc1ccc(NC(=O)[C@H]2CC[C@@H](N3Cc4c(C)ccc(OC)c4NC3=O)CC2)cc1C]

### (1s,4s)-N-(3-Methyl-4-methoxyphenyl)-4-(8-methoxy-5-methyl-2-oxo-1,2-dihydroquinazolin-3(4H)-yl)cyclohexanecarboxamide

Following the procedure described in Example 2 and making non-critical variations as required to replace 3-bromo-4-methoxyaniline with 3-methyl-4-methoxyaniline, ((1s,4s)-N-(3-Methyl-4-methoxyphenyl)-4-(8-methoxy-5-methyl-2-oxo-1,2-dihydroquinazolin-3(4H)-yl)cyclohexanecarboxamide was obtained as a white solid (23.4 mg, 14% Yield). LCMS ESI m/z: 438.4 [M+H]⁺. ¹H NMR (400 MHz, DMSO-d₆) δ 9.55 (s, 1H), 7.73 (s, 1H), 7.42-7.31 (m, 2H), 6.89-6.81 (m, 1H), 6.79-6.65 (m, 2H), 4.21 (s, 2H), 4.21-4.15 (m, 1H), 3.74 (d, J=1.7 Hz, 6H), 2.66-2.57 (m, 1H), 2.10 (d, J=2.3 Hz, 8H), 2.07-1.91 (m, 2H), 1.74-1.56 (m, 2H), 1.53-1.39 (m, 2H).

### Example 5

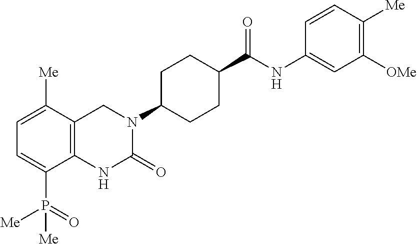 [SMILES: COc1cc(NC(=O)[C@H]2CC[C@@H](N3Cc4c(C)ccc(P(C)(C)=O)c4NC3=O)CC2)ccc1C]

### (1s,4s)-4-(8-(Dimethylphosphoryl)-5-methyl-2-oxo-1,2-dihydroquinazolin-3(4H)-yl)-N-(3-methoxy-4-methylphenyl)cyclohexanecarboxamide

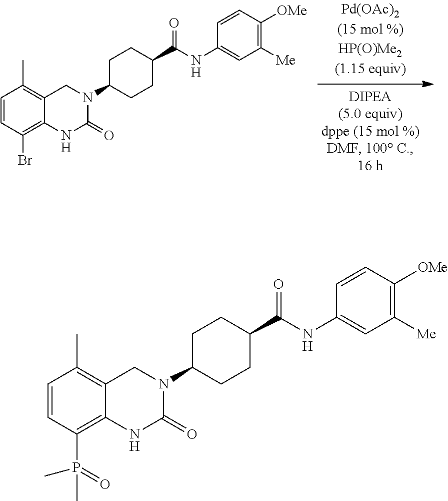 [SMILES: COc1ccc(NC(=O)[C@H]2CC[C@@H](N3Cc4c(C)ccc(Br)c4NC3=O)CC2)cc1C ; COc1ccc(NC(=O)[C@H]2CC[C@@H](N3Cc4c(C)ccc(P(C)(C)=O)c4NC3=O)CC2)cc1C]

### Step 1

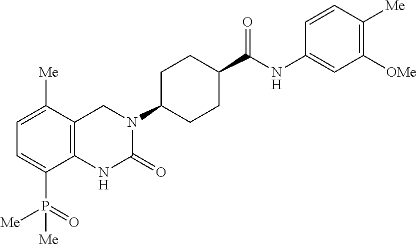 [SMILES: COc1cc(NC(=O)[C@H]2CC[C@@H](N3Cc4c(C)ccc(P(C)(C)=O)c4NC3=O)CC2)ccc1C]

### (1s,4s)-4-(8-(Dimethylphosphoryl)-5-methyl-2-oxo-1,2-dihydroquinazolin-3(4H)-yl)-N-(3-methoxy-4-methylphenyl)cyclohexanecarboxamide

To a microwave tube was added (1s,4s)-4-(8-bromo-5-methyl-2-oxo-1,2-dihydroquinazolin-3(4H)-yl)-N-(3-methoxy-4-methylphenyl)cyclohexanecarboxamide (50.0 mg, 84.3 μmol) and DMF (1.69 mL). The mixture was then purged with N₂ for 5 minutes. Then Pd(OAc)₂ (2.84 mg, 12.6 μmol), dimethylphosphine oxide (7.96 mg, 96.9 μmol), and 1,3-bis(diphenylphosphino) propane (5.38 mg, 12.6 μmol) were added to the mixture followed by N, N-Diisopropylethylamine (73.4 μL, 421 μmol). The mixture was then heated to 100° C. for 16 h. The crude reaction mixture was then quenched with NaHCO₃ (sat.), extracted with EtOAc(×3), washed with brine (×1), dried over Na₂SO₄ and concentrated under reduced pressure. The crude product was purified using reverse phase column (10 g, C18) using 0-80% MeCN:AMF (10 mM). The pure product was isolated after lyophilization as a pale yellow powder (21% yield, 8.49 mg). LCMS ESI m/z: 484.4 [M+H]⁺. ¹H NMR (400 MHz, DMSO-d₆) δ 10.12 (s, 1H), 9.71 (s, 1H), 8.32 (s, 1H), 7.31-7.22 (m, 2H), 7.11 (dd, J=8.1, 1.8 Hz, 1H), 7.02 (d, J=8.1 Hz, 1H), 6.83 (d, J=6.2 Hz, 1H), 4.24 (s, 2H), 4.18 (ddd, J=12.0, 7.6, 3.1 Hz, 1H), 3.75 (s, 3H), 2.65 (s, 1H), 2.20 (s, 3H), 2.16-2.09 (m, 2H), 2.08 (s, 3H), 2.06-1.94 (m, 2H), 1.78-1.68 (m, 6H), 1.68-1.60 (m, 1H), 1.51-1.41 (m, 2H).

### Examples 6 and Example 7

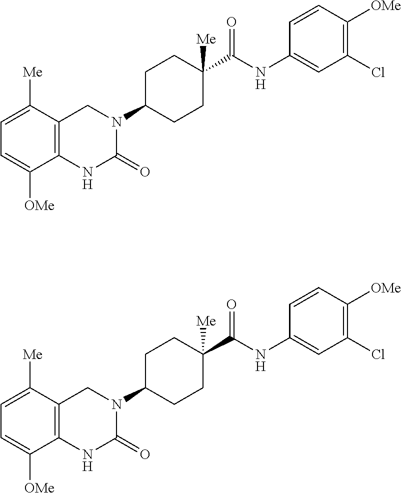 [SMILES: COc1ccc(NC(=O)[C@]2(C)CC[C@@H](N3Cc4c(C)ccc(OC)c4NC3=O)CC2)cc1Cl ; COc1ccc(NC(=O)[C@]2(C)CC[C@H](N3Cc4c(C)ccc(OC)c4NC3=O)CC2)cc1Cl]

### (1r,4r)-N-(3-Chloro-4-methoxyphenyl)-4-(8-methoxy-5-methyl-2-oxo-1,2-dihydroquinazolin-3(4H)-yl)-1-methylcyclohexanecarboxamide and (1s,4s)-N-(3-Chloro-4-methoxyphenyl)-4-(8-methoxy-5-methyl-2-oxo-1,2-dihydroquinazolin-3(4H)-yl)-1-methylcyclohexanecarboxamide

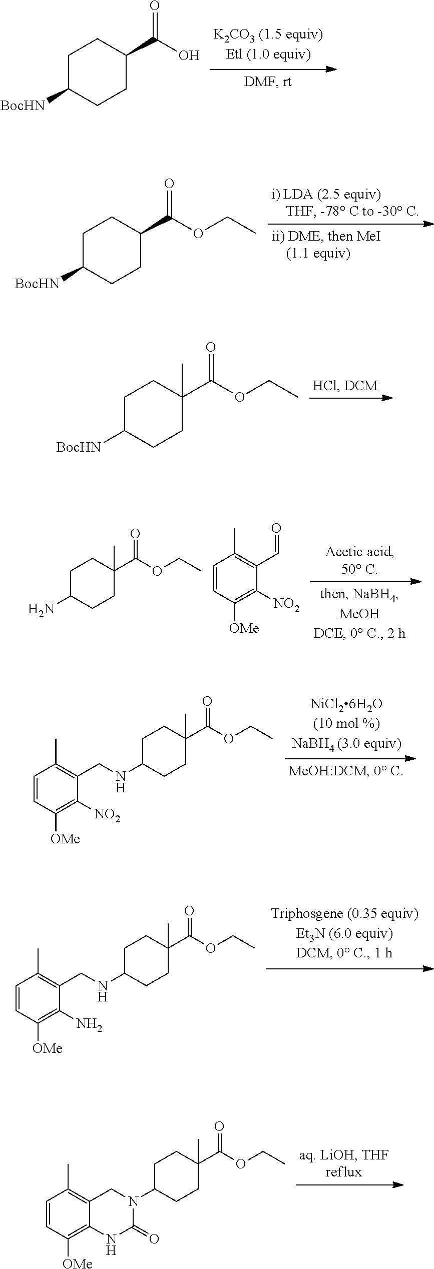

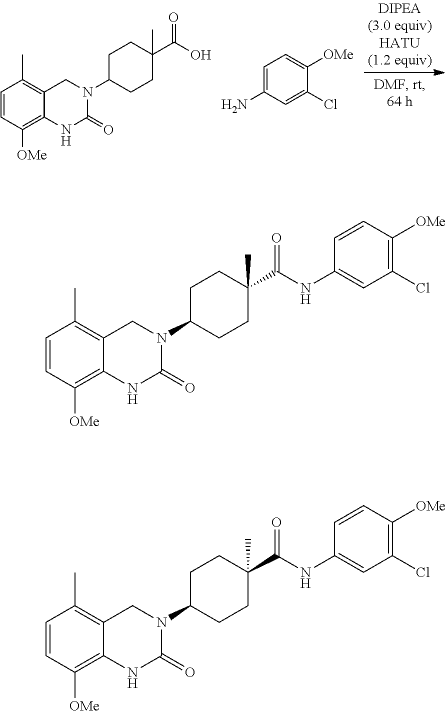 [SMILES: COc1ccc(C)c2c1NC(=O)N(C1CCC(C)(C(=O)O)CC1)C2 ; COc1ccc(N)cc1Cl ; COc1ccc(NC(=O)[C@]2(C)CC[C@@H](N3Cc4c(C)ccc(OC)c4NC3=O)CC2)cc1Cl ; COc1ccc(NC(=O)[C@]2(C)CC[C@H](N3Cc4c(C)ccc(OC)c4NC3=O)CC2)cc1Cl]

### Step 1

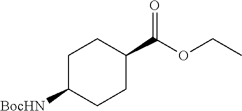 [SMILES: CCOC(=O)[C@H]1CC[C@@H](NC(=O)OC(C)(C)C)CC1]

### (1s,4s)-Ethyl 4-((tert-butoxycarbonyl)amino)cyclohexanecarboxylate

To a solution of cis-4-(Boc-amino)cyclohexanecarboxylic acid (5.00 g, 20.1 mmol) in DMF (50.3 mL) was added potassium carbonate (4.18 g, 30.2 mmol) and iodoethane (1.65 mL, 20.1 mmol) and the reaction mixture was stirred at room temperature for 16 h. Water was added and the product was extracted 3 times with EtOAc. Organics were combined, washed with water, brine, dried with Na₂SO₄, filtered and concentrated. The crude residue was purified by flash chromatography on silica gel (70 g column, 5 to 60% EtOAc in heptanes) to yield: (1s,4s)-Ethyl 4-((tert-butoxycarbonyl)amino)cyclohexanecarboxylate (5.73 g, 105% yield) as a colorless oil. ¹H NMR (400 MHz, CDCl₃) δ 4.62-4.51 (m, 1H), 4.13 (q, J=7.1 Hz, 2H), 3.70-3.58 (m, 1H), 2.43 (tt, J=7.7, 4.0 Hz, 1H), 1.89-1.78 (m, 2H), 1.76-1.63 (m, 4H), 1.60-1.56 (m, 1H), 1.43 (s, 9H), 1.25 (t, J=7.1 Hz, 3H).

### Step 2

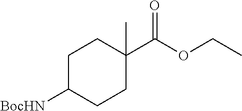 [SMILES: CCOC(=O)C1(C)CCC(NC(=O)OC(C)(C)C)CC1]

### cis and trans-Ethyl 4-((tert-butoxycarbonyl)amino)-1-methylcyclohexanecarboxylate

To a solution of diisopropylamine (389 μL, 2.78 mmol) dissolved in THF (2.4 mL) and cooled to 0° C. under N₂. n-Butyllithium (1.11 mL, 2.78 mmol) was added slowly. The mixture was stirred at 0° C. for 15 min and then cooled to −78° C. (1s,4s)-ethyl 4-((tert butoxycarbonyl)amino)cyclohexanecarboxylate (301 mg, 1.11 mmol) dissolved in THF (1.5 mL) was added. The mixture was warmed to −30° C. and stirred at this temperature for 30 min. Then DME (2.5 mL) was added to the solution, and the mixture was stirred at −60° C. for another 45 min. iodomethane (76.8 μL, 1.22 mmol) in THF (200 μL) was then added, and the solution was stirred for 1 hour at −60° C. The mixture was quenched with saturated aqueous NH₄Cl, and the pH was adjusted to 6 with 1 N aqueous HCl. The mixture was extracted with EtOAc (30 mL×2). The organic phase was washed with water (20 mL) and brine (15 mL×2), dried over Na₂SO₄ and concentrated in vacuo. The crude residue was purified by flash chromatography on silica gel (25 g column, 5 to 60% EtOAc in heptanes) to yield ethyl 4-((tert-butoxycarbonyl)amino)-1-methylcyclohexanecarboxylate (265 mg, 84% yield) as a pale yellow oil. Isolated as an inseparable mixture of diastereoisomers. LCMS ESI m/z 186.2 [M-Boc]⁺.

### Step 3

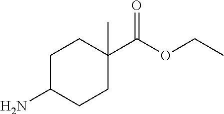

### Ethyl 4-amino-1-methylcyclohexanecarboxylate

To a solution of mixture of isomers of ethyl 4-((tert-butoxycarbonyl)amino)-1-methylcyclohexanecarboxylate (2.54 g, 8.90 mmol) in DCM (3 mL) was added hydrochloric acid (2.70 mL, 89.0 mmol) and the reaction mixture was stirred for 16 h. The reaction mixture was concentrated under reduced pressure and the crude solid was triturated in MTBE for 30 minutes. The white solid was filtered off. The solid was dissolved in DCM and washed with sat. NaHCO₃, organic layer was dried over Na₂SO₄, filtered and concentrated to yield: cis and trans-ethyl 4-amino-1-methylcyclohexanecarboxylate (1.12 g, 68% yield) as colorless oil. LCMS ESI m/z 186.3 [M+H]⁺.

### Step 4

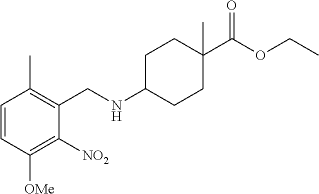 [SMILES: CCOC(=O)C1(C)CCC(NCc2c(C)ccc(OC)c2[N+](=O)[O-])CC1]

### Ethyl 4-((3-methoxy-6-methyl-2-nitrobenzyl)amino)-1-methylcyclohexanecarboxylate

To a solution of 3-methoxy-6-methyl-2-nitrobenzaldehyde (880 mg, 4.51 mmol) and ethyl 4-amino-1-methylcyclohexanecarboxylate (1.07 mL, 4.51 mmol) in DCE (15.0 mL) was added acetic acid (430 μL, 7.48 mmol) and the reaction mixture was stirred at 50° C. for 16 h. The reaction mixture was then cooled to 0° C. prior to the slow portion wise addition of sodium borohydride (266 mg, 6.76 mmol), followed by dropwise addition of 15 mL of MeOH. The reaction mixture was stirred from 0° C. to room temperature over 2 h. Upon completion of the reaction, a solution of ammonium chloride was added to the reaction mixture. The desired product was extracted 3 times with EtOAc. Organics were combined, washed with brine, dried with anhydrous sodium sulfate, filtered and concentrated to dryness. The crude residue was purified by flash chromatography on silica gel (80 g column, from 0 to 40% (20% MeOH in DCM) in DCM) to yield ethyl 4-((3-methoxy-6-methyl-2-nitrobenzyl)amino)-1-methylcyclohexanecarboxylate (1.34 g, 82% yield) as orange oil. LCMS ESI m/z 365.3 [M+H]⁺.

### Step 5

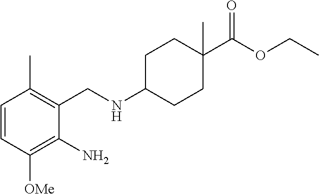 [SMILES: CCOC(=O)C1(C)CCC(NCc2c(C)ccc(OC)c2N)CC1]

### Ethyl 4-((2-amino-3-methoxy-6-methylbenzyl)amino)-1-methylcyclohexanecarboxylate

To a cold 0° C. solution of ethyl 4-((3-methoxy-6-methyl-2-nitrobenzyl)amino)-1-methylcyclohexanecarboxylate (1.30 g, 3.57 mmol) in DCM (43.7 mL) and MeOH (43.7 mL) (1:1 mixture) was added Nickel (II) chloride hexahydrate (87.3 mg, 367 μmol) followed by slow portion wise addition of sodium borohydride (422 mg, 10.7 mmol). The reaction mixture was stirred at 0° C. for 0.5 h and slowly turned black. A saturated solution of sodium bicarbonate was added to reaction mixture and it was extracted 3 times with DCM. Organics were combined, washed with brine, dried with anhydrous sodium sulfate, filtered and concentrated to dryness to provide ethyl 4-((2-amino-3-methoxy-6-methylbenzyl)amino)-1-methylcyclohexanecarboxylate (1.20 g, 100% yield) as crude product. LCMS ESI m/z 335.1 [M+H]⁺.

### Step 6

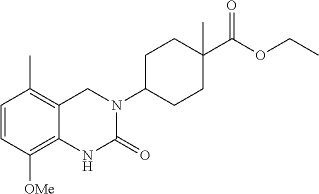 [SMILES: CCOC(=O)C1(C)CCC(N2Cc3c(C)ccc(OC)c3NC2=O)CC1]

### Ethyl 4-(8-methoxy-5-methyl-2-oxo-1,2-dihydroquinazolin-3(4H)-yl)-1-methylcyclohexanecarboxylate

To a solution of ethyl 4-((2-amino-3-methoxy-6-methylbenzyl)amino)-1-methylcyclohexanecarboxylate (1.15 g, 3.44 mmol) in DCM (160 mL) was added Triphosgene (364 mg, 1.20 mmol) followed by dropwise addition of a solution of triethylamine (2.89 mL, 20.6 mmol) in 0.5 mL of DCM. The reaction mixture was stirred at room temperature for 2 h. After complete conversion of the starting material the reaction mixture was concentrated to dryness and the crude residue was purified by flash chromatography on silica gel (50 g column, from 1 to 60% (20% MeOH in DCM) in DCM) to yield ethyl 4-(8-methoxy-5-methyl-2-oxo-1,2-dihydroquinazolin-3(4H)-yl)-1-methylcyclohexanecarboxylate (1.00 g, 81% yield) as a beige solid. LCMS ESI m/z 361.3 [M+H]⁺.

### Step 7

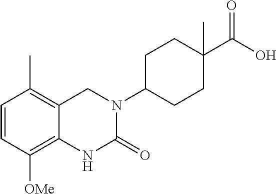 [SMILES: COc1ccc(C)c2c1NC(=O)N(C1CCC(C)(C(=O)O)CC1)C2]

### 4-(8-Methoxy-5-methyl-2-oxo-1,2-dihydroquinazolin-3(4H)-yl)-1-methylcyclohexanecarboxylic Acid

To a solution of ethyl 4-(8-methoxy-5-methyl-2-oxo-1,2-dihydroquinazolin-3(4H)-yl)-1-methylcyclohexanecarboxylate (1.00 g, 2.77 mmol) in THF was added lithium hydroxide (5.55 mL, 5.55 mmol) and the reaction mixture was stirred at 100° C. for 16 h. The reaction mixture was transferred into a microwave vial and heat to 170° C. for 2×30 min under microwave radiation. Once cooled to rt, the solution was acidified using 1M HCl and the desired product extracted 3 times with EtOAc. Organics were combined, washed with water, brine, dried with Na₂SO₄, filtered and concentrated to yield 4-(8-methoxy-5-methyl-2-oxo-1,2-dihydroquinazolin-3(4H)-yl)-1-methylcyclohexanecarboxylic acid (454 mg, 49% yield) pale yellow solid. LCMS ESI m/z 333.1 [M+H]⁺.

### Step 8

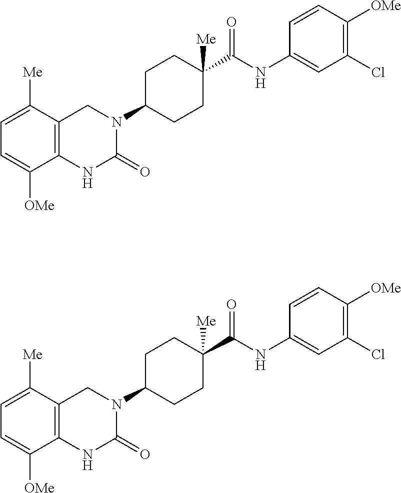 [SMILES: COc1ccc(NC(=O)[C@]2(C)CC[C@@H](N3Cc4c(C)ccc(OC)c4NC3=O)CC2)cc1Cl ; COc1ccc(NC(=O)[C@]2(C)CC[C@H](N3Cc4c(C)ccc(OC)c4NC3=O)CC2)cc1Cl]

### (1r,4r)-N-(3-Chloro-4-methoxyphenyl)-4-(8-methoxy-5-methyl-2-oxo-1,2-dihydroquinazolin-3(4H)-yl)-1-methylcyclohexanecarboxamide and (1s,4s)-N-(3-Chloro-4-methoxyphenyl)-4-(8-methoxy-5-methyl-2-oxo-1,2-dihydroquinazolin-3(4H)-yl)-1-methylcyclohexanecarboxamide

To a solution of 4-(8-methoxy-5-methyl-2-oxo-1,2-dihydroquinazolin-3(4H)-yl)-1-methylcyclohexanecarboxylic acid (114 mg, 341 μmol) and 3-chloro-4-methoxyaniline (170 mg, 1.04 mmol) in DMF (2.00 mL) was added N,N-diisopropylethylamine (180 μL, 1.02 mmol) and finally, HATU (159 mg, 410 μmol). The reaction mixture was stirred for 64 hours. The reaction mixture was diluted with 1:1 NH₄Cl (sat) and water and it was extracted 3 times with EtOAc. Organics were combined, washed with water, brine, dried with anhydrous sodium sulfate, filtered and concentrated to dryness. Crude residue was purified by reverse phase flash chromatography on C18 (120 g column, from 0 to 60% MeCN in water-10 mM Ammonium formate pH=3.8). Pure fractions were directly lyophilized to yield (1r,4r)-N-(3-Chloro-4-methoxyphenyl)-4-(8-methoxy-5-methyl-2-oxo-1,2-dihydroquinazolin-3(4H)-yl)-1-methylcyclohexanecarboxamide (Example 6) (38.7 mg, 24% yield) as a white solid. LCMS ESI m/z 472.4 [M+H]⁺. ¹H NMR (400 MHz, dmso-d₆) δ 9.34 (s, 1H), 7.76 (d, J=2.5 Hz, 1H), 7.71 (s, 1H), 7.51 (dd, J=8.9, 2.5 Hz, 1H), 7.09 (d, J=9.0 Hz, 1H), 6.72 (d, J=8.3 Hz, 1H), 6.64 (d, J=8.5 Hz, 1H), 4.20-4.13 (m, 1H), 4.12 (s, 2H), 3.80 (s, 3H), 3.71 (s, 3H), 2.34 (d, J=12.8 Hz, 2H), 2.03 (s, 3H), 1.75-1.60 (m, 2H), 1.48 (d, J=10.1 Hz, 2H), 1.31 (t, J=12.2 Hz, 2H), 1.17 (s, 3H) and (1s,4s)-N-(3-chloro-4-methoxyphenyl)-4-(8-methoxy-5-methyl-2-oxo-1,2-dihydroquinazolin-3(4H)-yl)-1-methylcyclohexanecarboxamide (Example 7) (57.2 mg, 35% yield) as white solid. LCMS ESI m/z 472.4 [M+H]⁺. ¹H NMR (400 MHz, DMSO-d6) δ 9.25 (s, 1H), 7.78 (d, J=2.5 Hz, 1H), 7.73 (s, 1H), 7.53 (dd, J=9.0, 2.5 Hz, 1H), 7.07 (d, J=9.1 Hz, 1H), 6.74 (d, J=8.3 Hz, 1H), 6.67 (d, J=8.3 Hz, 1H), 4.30 (s, 2H), 4.17-4.06 (m, 1H), 3.79 (s, 3H), 3.72 (s, 3H), 2.15 (s, 3H), 1.95-1.82 (m, 2H), 1.81-1.74 (m, 4H), 1.49 (dd, J=11.1, 2.5 Hz, 2H), 1.29 (s, 3H). Arbitrary stereoisomer assignment of (1r,4r) to Example 6 and (1s,4s) to Example 7.

### Examples 8 and Example 9

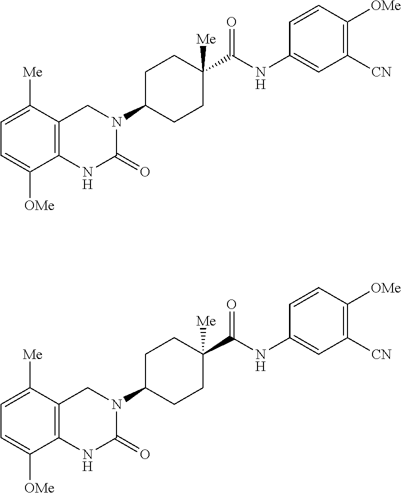 [SMILES: COc1ccc(NC(=O)[C@]2(C)CC[C@@H](N3Cc4c(C)ccc(OC)c4NC3=O)CC2)cc1C#N ; COc1ccc(NC(=O)[C@]2(C)CC[C@H](N3Cc4c(C)ccc(OC)c4NC3=O)CC2)cc1C#N]

### (1r,4r)-N-(3-Cyano-4-methoxyphenyl)-4-(8-methoxy-5-methyl-2-oxo-1,2-dihydroquinazolin-3(4H)-yl)-1-methylcyclohexanecarboxamide and (1s,4s)-N-(3-Cyano-4-methoxyphenyl)-4-(8-methoxy-5-methyl-2-oxo-1,2-dihydroquinazolin-3(4H)-yl)-1-methylcyclohexanecarboxamide

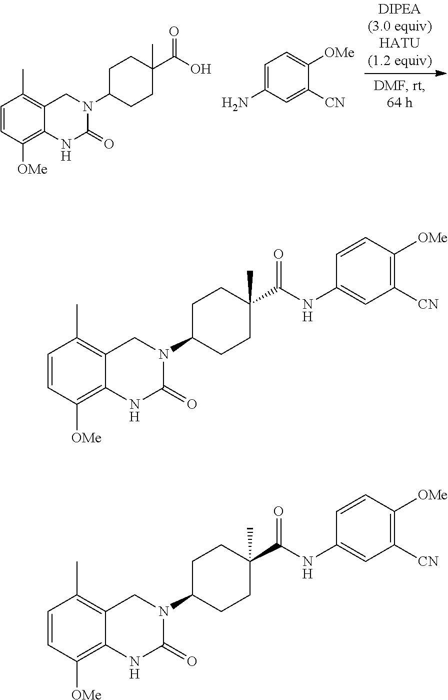 [SMILES: COc1ccc(C)c2c1NC(=O)N(C1CCC(C)(C(=O)O)CC1)C2 ; COc1ccc(N)cc1C#N ; COc1ccc(NC(=O)[C@]2(C)CC[C@@H](N3Cc4c(C)ccc(OC)c4NC3=O)CC2)cc1C#N ; COc1ccc(NC(=O)[C@]2(C)CC[C@H](N3Cc4c(C)ccc(OC)c4NC3=O)CC2)cc1C#N]

### Step 1

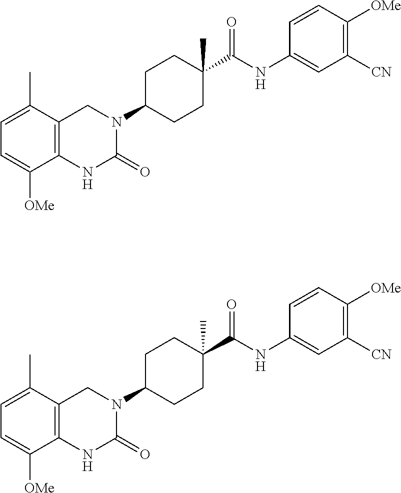 [SMILES: COc1ccc(NC(=O)[C@]2(C)CC[C@@H](N3Cc4c(C)ccc(OC)c4NC3=O)CC2)cc1C#N ; COc1ccc(NC(=O)[C@]2(C)CC[C@H](N3Cc4c(C)ccc(OC)c4NC3=O)CC2)cc1C#N]

### (1r,4r)-N-(3-Cyano-4-methoxyphenyl)-4-(8-methoxy-5-methyl-2-oxo-1,2-dihydroquinazolin-3(4H)-yl)-1-methylcyclohexanecarboxamide and (1s,4s)-N-(3-Cyano-4-methoxyphenyl)-4-(8-methoxy-5-methyl-2-oxo-1,2-dihydroquinazolin-3(4H)-yl)-1-methylcyclohexanecarboxamide

To a solution of 4-(8-methoxy-5-methyl-2-oxo-1,2-dihydroquinazolin-3(4H)-yl)-1-methylcyclohexanecarboxylic acid (114 mg, 341 μmol) and 5-amino-2-methoxybenzonitrile (158 mg, 1.04 mmol) in DMF (2.00 mL) was added N,N-diisopropylethylamine (180 μL, 1.02 mmol) and finally, HATU (159 mg, 410 μmol). The reaction mixture was stirred for 64 hours. The reaction mixture was diluted with 1:1 NH₄Cl (sat) and water and it was extracted 3 times with EtOAc. Organics were combined, washed with water, brine, dried with anhydrous sodium sulfate, filtered and concentrated to dryness. Crude residue was purified by reverse phase flash chromatography on C18 (80 g column, 0 to 60% MeCN in water-10 mM Ammonium formate pH=3.8). Pure fractions were directly lyophilized to yield (1r,4r)-N-(3-cyano-4-methoxyphenyl)-4-(8-methoxy-5-methyl-2-oxo-1,2-dihydroquinazolin-3(4H)-yl)-1-methylcyclohexanecarboxamide (Example 8) (30.7 mg, 19% yield) as off white solid. LCMS ESI m/z 463.4 [M+H]⁺; ¹H NMR (400 MHz, DMSO-d₆) δ 9.54 (s, 1H), 8.36 (s, 1H), 7.97 (d, J=2.6 Hz, 1H), 7.84 (dd, J=9.2, 2.6 Hz, 1H), 7.72 (s, 1H), 7.22 (d, J=9.2 Hz, 1H), 6.72 (d, J=8.3 Hz, 1H), 6.64 (d, J=8.5 Hz, 1H), 4.20-4.13 (m, 1H), 4.12 (s, 2H), 3.87 (s, 3H), 3.71 (s, 3H), 2.35 (d, J=12.9 Hz, 2H), 2.03 (s, 3H), 1.68 (q, J=12.2 Hz, 2H), 1.48 (d, J=10.5 Hz, 2H), 1.32 (t, J=12.0 Hz, 2H), 1.17 (s, 3H) and (1s,4s)-N-(3-cyano-4-methoxyphenyl)-4-(8-methoxy-5-methyl-2-oxo-1,2-dihydroquinazolin-3(4H)-yl)-1-methylcyclohexanecarboxamide (Example 9) (85.4 mg, 54% yield) as off white solid formate salt; LCMS ESI m/z 463.4 [M+H]⁺; ¹H NMR (400 MHz, DMSO-d₆) δ 9.39 (s, 1H), 7.97 (d, J=2.6 Hz, 1H), 7.85 (dd, J=9.2, 2.6 Hz, 1H), 7.73 (s, 1H), 7.20 (d, J=9.3 Hz, 1H), 6.74 (d, J=8.3 Hz, 1H), 6.67 (d, J=8.3 Hz, 1H), 4.31 (s, 2H), 4.12 (tt, J=11.8, 3.6 Hz, 1H), 3.86 (s, 3H), 3.72 (s, 3H), 2.15 (s, 3H), 1.97-1.82 (m, 2H), 1.81-1.74 (m, 4H), 1.55-1.46 (m, 2H), 1.30 (s, 3H). Arbitrary stereoisomer assignment of (1r,4r) to Example 8 and (1s,4s) to Example 9.

### Examples 10 and Example 11

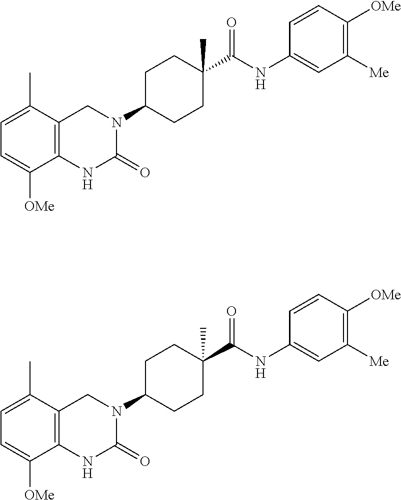 [SMILES: COc1ccc(NC(=O)[C@]2(C)CC[C@@H](N3Cc4c(C)ccc(OC)c4NC3=O)CC2)cc1C ; COc1ccc(NC(=O)[C@]2(C)CC[C@H](N3Cc4c(C)ccc(OC)c4NC3=O)CC2)cc1C]

### (1r,4r)-N-(3-Methyl-4-methoxyphenyl)-4-(8-methoxy-5-methyl-2-oxo-1,2-dihydroquinazolin-3(4H)-yl)-1-methylcyclohexanecarboxamide and (1s,4s)-N-(3-Methyl-4-methoxyphenyl)-4-(8-methoxy-5-methyl-2-oxo-1,2-dihydroquinazolin-3(4H)-yl)-1-methylcyclohexanecarboxamide

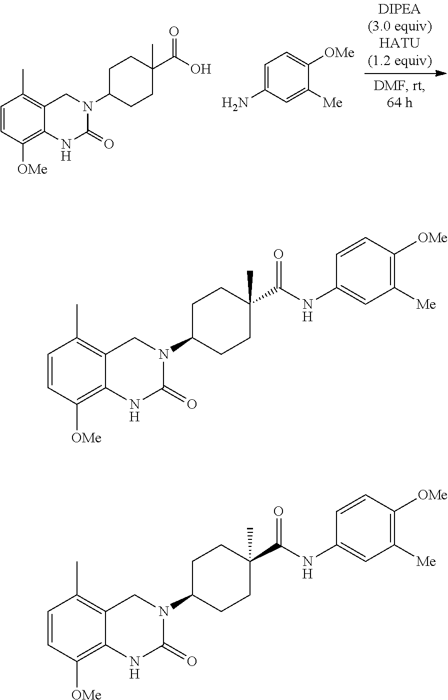 [SMILES: COc1ccc(C)c2c1NC(=O)N(C1CCC(C)(C(=O)O)CC1)C2 ; COc1ccc(N)cc1C ; COc1ccc(NC(=O)[C@]2(C)CC[C@@H](N3Cc4c(C)ccc(OC)c4NC3=O)CC2)cc1C ; COc1ccc(NC(=O)[C@]2(C)CC[C@H](N3Cc4c(C)ccc(OC)c4NC3=O)CC2)cc1C]

### Step 1

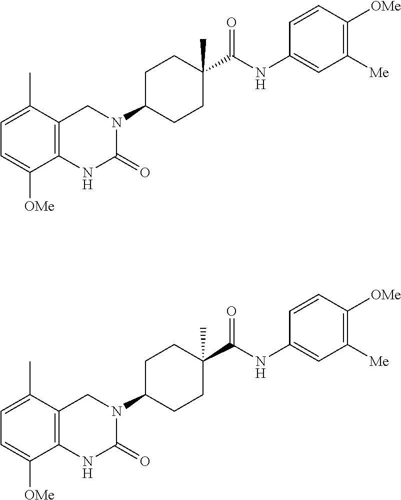 [SMILES: COc1ccc(NC(=O)[C@]2(C)CC[C@@H](N3Cc4c(C)ccc(OC)c4NC3=O)CC2)cc1C ; COc1ccc(NC(=O)[C@]2(C)CC[C@H](N3Cc4c(C)ccc(OC)c4NC3=O)CC2)cc1C]

### (1r,4r)-N-(3-Methyl-4-methoxyphenyl)-4-(8-methoxy-5-methyl-2-oxo-1,2-dihydroquinazolin-3(4H)-yl)-1-methylcyclohexanecarboxamide and (1s,4s)-N-(3-Methyl-4-methoxyphenyl)-4-(8-methoxy-5-methyl-2-oxo-1,2-dihydroquinazolin-3(4H)-yl)-1-methylcyclohexanecarboxamide

To a solution of 4-(8-methoxy-5-methyl-2-oxo-1,2-dihydroquinazolin-3(4H)-yl)-1-methylcyclohexanecarboxylic acid (114 mg, 341 μmol) and 4-methoxy-3-methylaniline (143 mg, 1.02 mmol) in DMF (2.00 mL) was added N,N-diisopropylethylamine (180 μL, 1.02 mmol) and finally, HATU (159 mg, 410 μmol). The reaction mixture was stirred for 64 hours. The reaction mixture was diluted with 1:1 NH₄Cl (sat) and water and it was extracted 3 times with EtOAc. Organics were combined, washed with water, brine, dried with anhydrous sodium sulfate, filtered and concentrated to dryness. Crude residue was purified by reverse phase flash chromatography on C18 (80 g column, 0 to 60% MeCN in water-10 mM Ammonium formate pH=3.8). Pure fractions were directly lyophilized to yield (1r,4r)-N-(3-methyl-4-methoxyphenyl)-4-(8-methoxy-5-methyl-2-oxo-1,2-dihydroquinazolin-3(4H)-yl)-1-methylcyclohexanecarboxamide (Example 10) (52.4 mg, 34% yield) as off white solid; LCMS ESI m/z 452.4 [M+H]⁺; ¹H NMR (400 MHz, DMSO-d₆) δ 9.03 (s, 1H), 7.72 (s, 1H), 7.41-7.34 (m, 2H), 6.82 (d, J=8.6 Hz, 1H), 6.74 (d, J=8.3 Hz, 1H), 6.67 (d, J=8.3 Hz, 1H), 4.30 (s, 2H), 4.17-4.06 (m, 1H), 3.72 (s, 6H), 2.14 (s, 3H), 2.10 (s, 3H), 1.95-1.81 (m, 2H), 1.81-1.74 (m, 4H), 1.49 (d, J=8.9 Hz, 2H), 1.28 (s, 3H); and (1s,4s)-N-(3-methyl-4-methoxyphenyl)-4-(8-methoxy-5-methyl-2-oxo-1,2-dihydroquinazolin-3(4H)-yl)-1-methylcyclohexanecarboxamide (Example 11) (29.9 mg, 19% yield) as a beige solid. LCMS ESI m/z 452.3 [M+H]⁺; ¹H NMR (400 MHz, DMSO-d₆) δ 9.14 (s, 1H), 7.71 (s, 1H), 7.38-7.31 (m, 2H), 6.85 (d, J=9.4 Hz, 1H), 6.72 (d, J=8.3 Hz, 1H), 6.64 (d, J=8.4 Hz, 1H), 4.22-4.13 (m, 1H), 4.12 (s, 2H), 3.73 (s, 3H), 3.71 (s, 3H), 2.35 (d, J=12.8 Hz, 2H), 2.11 (s, 3H), 2.03 (s, 3H), 1.76-1.61 (m, 2H), 1.47 (d, J=10.2 Hz, 2H), 1.35-1.22 (m, 2H), 1.16 (s, 3H). Arbitrary stereoisomer assignment of (1r,4r) to Example 10 and (1s,4s) to Example 11.

### Examples 12 and 13

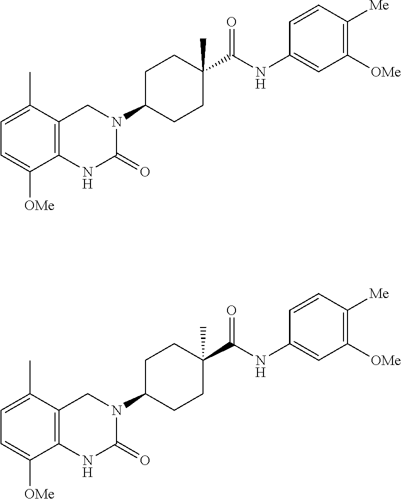 [SMILES: COc1cc(NC(=O)[C@]2(C)CC[C@@H](N3Cc4c(C)ccc(OC)c4NC3=O)CC2)ccc1C ; COc1cc(NC(=O)[C@]2(C)CC[C@H](N3Cc4c(C)ccc(OC)c4NC3=O)CC2)ccc1C]

### (1r,4r)-N-(3-Methoxy-4-methylphenyl)-4-(8-methoxy-5-methyl-2-oxo-1,2-dihydroquinazolin-3(4H)-yl)-1-methylcyclohexanecarboxamide and (1s,4s)-N-(3-Methoxy-4-methylphenyl)-4-(8-methoxy-5-methyl-2-oxo-1,2-dihydroquinazolin-3(4H)-yl)-1-methylcyclohexanecarboxamide

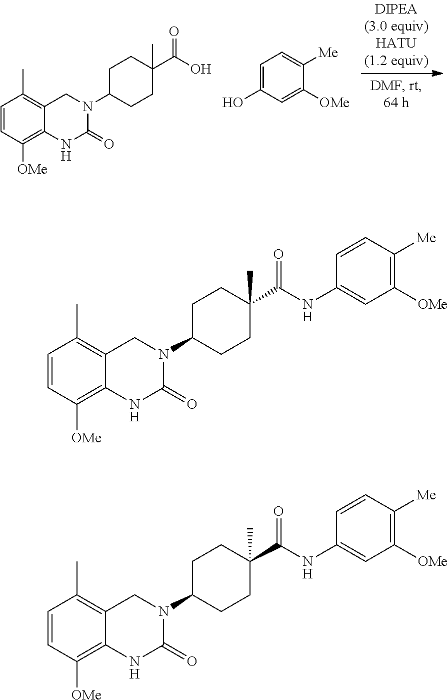 [SMILES: COc1ccc(C)c2c1NC(=O)N(C1CCC(C)(C(=O)O)CC1)C2 ; COc1cc(O)ccc1C ; COc1cc(NC(=O)[C@]2(C)CC[C@@H](N3Cc4c(C)ccc(OC)c4NC3=O)CC2)ccc1C ; COc1cc(NC(=O)[C@]2(C)CC[C@H](N3Cc4c(C)ccc(OC)c4NC3=O)CC2)ccc1C]

### Step 1

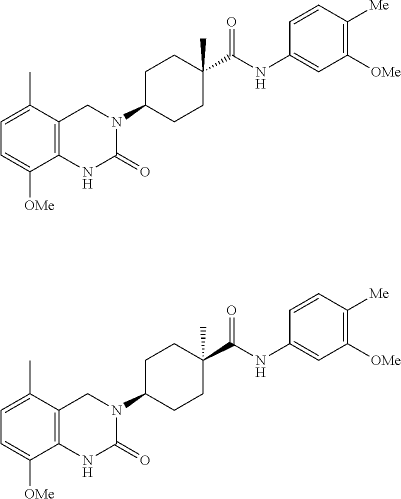 [SMILES: COc1cc(NC(=O)[C@]2(C)CC[C@@H](N3Cc4c(C)ccc(OC)c4NC3=O)CC2)ccc1C ; COc1cc(NC(=O)[C@]2(C)CC[C@H](N3Cc4c(C)ccc(OC)c4NC3=O)CC2)ccc1C]

### (1r,4r)-N-(3-Methoxy-4-methylphenyl)-4-(8-methoxy-5-methyl-2-oxo-1,2-dihydroquinazolin-3(4H)-yl)-1-methylcyclohexanecarboxamide and (1s,4s)-N-(3-Methoxy-4-methylphenyl)-4-(8-methoxy-5-methyl-2-oxo-1,2-dihydroquinazolin-3(4H)-yl)-1-methylcyclohexanecarboxamide

To a solution of 4-(8-methoxy-5-methyl-2-oxo-1,2-dihydroquinazolin-3(4H)-yl)-1-methylcyclohexanecarboxylic acid (114 mg, 341 μmol) and 4-methyl-3-methoxyaniline (143 mg, 1.02 mmol) in DMF (2.00 mL) was added N,N-diisopropylethylamine (180 μL, 1.02 mmol) and finally, HATU (159 mg, 410 μmol). The reaction mixture was stirred for 64 hours. The reaction mixture was diluted with 1:1 NH₄Cl (sat) and water and it was extracted 3 times with EtOAc. Organics were combined, washed with water, brine, dried with anhydrous sodium sulfate, filtered and concentrated to dryness. Crude residue was purified by reverse phase flash chromatography on C18 (80 g column, 0 to 60% MeCN in water-10 mM Ammonium formate pH=3.8). Pure fractions were directly lyophilized to yield (1r,4r)-N-(3-methyl-4-methoxyphenyl)-4-(8-methoxy-5-methyl-2-oxo-1,2-dihydroquinazolin-3(4H)-yl)-1-methylcyclohexanecarboxamide (Example 12) (20.8 mg, 13% yield) as beige solid; LCMS ESI m/z 452.4 [M+H]⁺; ¹H NMR (400 MHz, DMSO-d₆) δ 9.15 (s, 1H), 7.73 (s, 1H), 7.33 (d, J=1.7 Hz, 1H), 7.17 (dd, J=8.1, 1.8 Hz, 1H), 6.99 (d, J=8.4 Hz, 1H), 6.74 (d, J=8.3 Hz, 1H), 6.67 (d, J=8.4 Hz, 1H), 4.31 (s, 2H), 4.13 (tt, J=12.0, 3.7 Hz, 1H), 3.73 (s, 3H), 3.72 (s, 3H), 2.15 (s, 3H), 2.06 (s, 3H), 1.95-1.83 (m, 2H), 1.82-1.75 (m, 4H), 1.50 (dd, J=11.8, 2.9 Hz, 2H), 1.30 (s, 3H); and (1s,4s)-N-(3-methyl-4-methoxyphenyl)-4-(8-methoxy-5-methyl-2-oxo-1,2-dihydroquinazolin-3(4H)-yl)-1-methylcyclohexanecarboxamide (Example 13) (33.1 mg, 21% yield) as a white solid. LCMS ESI m/z 452.4 [M+H]⁺; ¹H NMR (400 MHz, DMSO-d₆) δ 9.25 (s, 1H), 7.72 (s, 1H), 7.29 (d, J=1.7 Hz, 1H), 7.17 (dd, J=8.0, 1.8 Hz, 1H), 7.01 (d, J=8.4 Hz, 1H), 6.72 (d, J=8.3 Hz, 1H), 6.64 (d, J=8.5 Hz, 1H), 4.17 (tt, J=12.3, 3.3 Hz, 1H), 4.12 (s, 2H), 3.74 (s, 3H), 3.71 (s, 3H), 2.37 (d, J=12.7 Hz, 2H), 2.07 (s, 3H), 2.02 (s, 3H), 1.68 (qd, J=13.1, 2.0 Hz, 2H), 1.48 (dd, J=11.4, 1.8 Hz, 2H), 1.35-1.26 (m, 2H), 1.18 (s, 3H).

### Examples 14 and Example 15

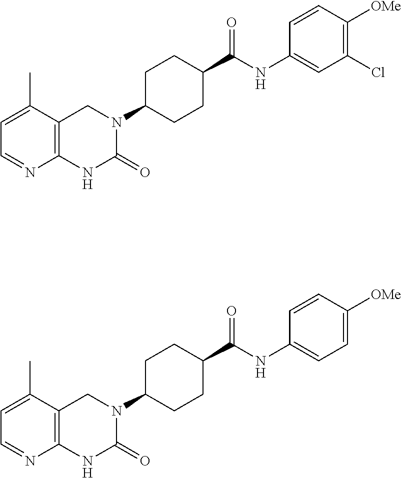 [SMILES: COc1ccc(NC(=O)[C@H]2CC[C@@H](N3Cc4c(C)ccnc4NC3=O)CC2)cc1Cl ; COc1ccc(NC(=O)[C@H]2CC[C@@H](N3Cc4c(C)ccnc4NC3=O)CC2)cc1]

### (1s,4s)-N-(3-Chloro-4-methoxyphenyl)-4-(5-methyl-2-oxo-1,2-dihydropyrido[2,3-d]pyrimidin-3(4H)-yl)cyclohexanecarboxamide and (1s,4s)-N-(4-Methoxyphenyl)-4-(5-methyl-2-oxo-1,2-dihydropyrido[2,3-d]pyrimidin-3(4H)-yl)cyclohexanecarboxamide

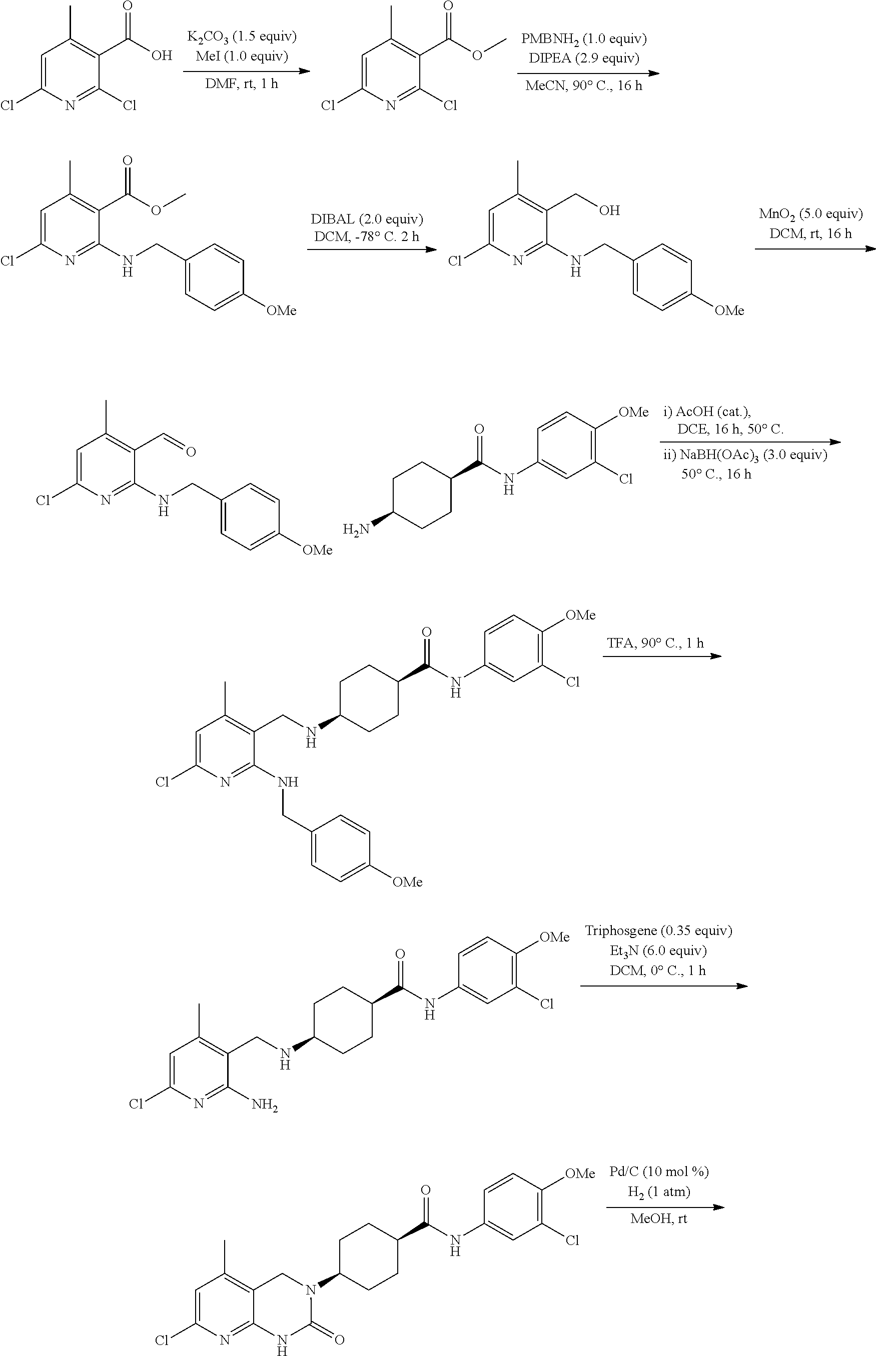

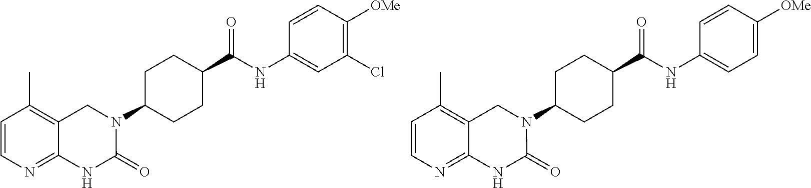 [SMILES: COc1ccc(NC(=O)[C@H]2CC[C@@H](N3Cc4c(C)ccnc4NC3=O)CC2)cc1Cl ; COc1ccc(NC(=O)[C@H]2CC[C@@H](N3Cc4c(C)ccnc4NC3=O)CC2)cc1]

### Step 1

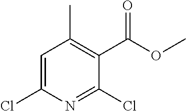

### Methyl 2,6-dichloro-4-methylnicotinate

To a solution of 2,6-dichloro-4-methylnicotinic acid (2.00 g, 9.71 mmol) in DMF (24.3 mL) was added potassium carbonate (2.01 g, 14.6 mmol) and iodomethane (610 μL, 9.71 mmol) and the reaction mixture was stirred at room temperature for 1 h. Water was added and the product was extracted 3 times with EtOAc. Organics were combined, washed with water, brine, dried with Na₂SO₄, filtered and concentrated. The crude residue was purified by flash chromatography on silica gel (70 g column, 5 to 60% EtOAc in heptanes) to yield methyl 2,6-dichloro-4-methylnicotinate (2.13 g, 100% yield) as a white solid. LCMS ESI m/z 220.0 [M+H]⁺.

### Step 2

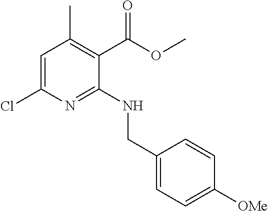 [SMILES: COC(=O)c1c(C)cc(Cl)nc1NCc1ccc(OC)cc1]

### Methyl 6-chloro-2-((4-methoxybenzyl)amino)-4-methylnicotinate

To a cold 0° C. solution of methyl 2,6-dichloro-4-methylnicotinate (825 mg, 3.75 mmol) in MeCN (7.50 mL) was added N,N-diisopropylethylamine (1.90 mL, 10.8 mmol) and 4-methoxybenzylamine (500 uL, 3.75 mmol) and the reaction mixture was stirred at 0° C. for 10 minutes. Temperature was raised to 90° C. overnight (16 h). The reaction was stopped, concentrated to dryness and directly purified by flash chromatography on silica gel (50 g column, 0 to 45% EtOAc in heptanes) to yield methyl 6-chloro-2-((4-methoxybenzyl)amino)-4-methylnicotinate (1.01 g, 84% yield) as a white solid. LCMS ESI m/z 320.9 [M+H]⁺. ¹H NMR (400 MHz, CDCl₃) δ 8.27 (s, 1H), 7.29 (d, J=8.7 Hz, 2H), 6.86 (d, J=8.7 Hz, 2H), 6.40 (s, 1H), 4.59 (s, 2H), 3.84 (s, 3H), 3.79 (s, 3H), 2.42 (s, 3H).

### Step 3

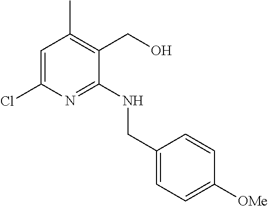 [SMILES: COc1ccc(CNc2nc(Cl)cc(C)c2CO)cc1]

### (6-Chloro-2-((4-methoxybenzyl)amino)-4-methylpyridin-3-yl)methanol

To a −78° C. solution of methyl 4-((4-methoxybenzyl)amino)-6-methylpyrimidine-5-carboxylate (641 mg, 2.00 mmol) in DCM (9.99 mL) under nitrogen atmosphere was dropwise added a 1M solution of diisobutylaluminum hydride (4.00 mL, 4.00 mmol) in toluene. The reaction mixture was stirred at −78° C. for 2 h. To the reaction mixture was added a 1M solution of HCl at −78° C. and the reaction mixture was allowed to warm to room temperature. The reaction mixture was extracted twice with DCM and once with a 20% IPA/CHCl₃ mixture. Organics were combined, washed with brine, dried with anhydrous sodium sulfate, filtered and concentrated to dryness. The crude residue was purified by flash chromatography on silica gel (50 g column, 30 to 80% EtOAc in heptanes) to yield (6-chloro-2-((4-methoxybenzyl)amino)-4-methylpyridin-3-yl)methanol (451 mg, 80% yield) as a off white solid. LCMS ESI m/z 292.9 [M+H]⁺.

### Step 4

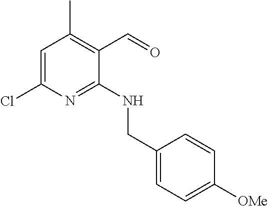 [SMILES: COc1ccc(CNc2nc(Cl)cc(C)c2C=O)cc1]

### 6-Chloro-2-((4-methoxybenzyl)amino)-4-methylnicotinaldehyde

To a solution of (6-chloro-2-((4-methoxybenzyl)amino)-4-methylpyridin-3-yl)methanol (430 mg, 1.47 mmol) in DCM (7.34 mL) was added manganese (IV) oxide activated (709 mg, 7.34 mmol) and the reaction mixture was stirred at room temperature for 16 h. The reaction mixture was filtered through Celite and rinsed with DCM. The filtrate was concentrated to dryness. The crude residue was purified by flash chromatography on silica gel (50 g column, 5 to 60% EtOAc in heptanes) to yield 6-Chloro-2-((4-methoxybenzyl)amino)-4-methylnicotinaldehyde (293 mg, 69% yield) as a pale yellow solid. LCMS ESI m/z 290.9 [M+H]⁺.

### Step 5

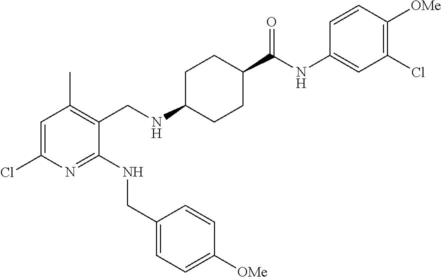 [SMILES: COc1ccc(CNc2nc(Cl)cc(C)c2CN[C@H]2CC[C@@H](C(=O)Nc3ccc(OC)c(Cl)c3)CC2)cc1]

### (1s,4s)-4-(((6-Chloro-2-((4-methoxybenzyl)amino)-4-methylpyridin-3-yl)methyl)amino)-N-(3-chloro-4-methoxyphenyl)cyclohexanecarboxamide

6-Chloro-2-((4-methoxybenzyl)amino)-4-methylnicotinaldehyde (225 mg, 774 μmol) and (1s,4s)-4-amino-N-(3-chloro-4-methoxyphenyl)cyclohexanecarboxamide (225 mg, 797 μmol) were place in a vial and DCE (5.16 mL) was added followed by a drop of acetic acid. The reagents were mixed for 16 hour at 50° C. Sodium triacetoxyborohydride (490 mg, 2.32 mmol) was added in one portion. The reaction mixture was stirred at 50° C. for 16 h. MeOH was added to the reaction mixture and the resulting solution was stirred at room temperature for 1h. Concentrated to dryness. The crude residue was purified by flash chromatography on silica gel (from 1 to 60% (20% MeOH in DCM) in DCM) to yield (1s,4s)-4-(((6-chloro-2-((4-methoxybenzyl)amino)-4-methylpyridin-3-yl)methyl)amino)-N-(3-chloro-4-methoxyphenyl)cyclohexanecarboxamide (255 mg, 59% yield) as a pale yellow solid. LCMS ESI m/z 556.9, 558.8 [M+H]⁺.

### Step 6

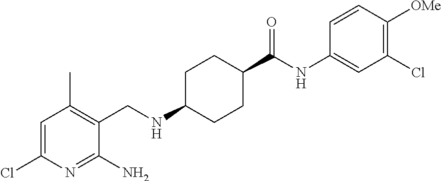 [SMILES: COc1ccc(NC(=O)[C@H]2CC[C@@H](NCc3c(C)cc(Cl)nc3N)CC2)cc1Cl]

### (1s,4s)-4-(((2-Amino-6-chloro-4-methylpyridin-3-yl)methyl)amino)-N-(3-chloro-4-methoxyphenyl)cyclohexanecarboxamide

To a vial loaded with (1s,4s)-4-(((6-chloro-2-((4-methoxybenzyl)amino)-4-methylpyridin-3-yl)methyl)amino)-N-(3-chloro-4-methoxyphenyl)cyclohexanecarboxamide (160 mg, 287 μmol) was added trifluoroacetic acid (220 μL, 2.87 mmol) and the reaction mixture was stirred at 90° C. for 1 h. The reaction mixture was concentrated to dryness and a saturated solution of sodium bicarbonate was added to reaction mixture and it was extracted 3 times with DCM. Organics were combined, washed with brine, dried with anhydrous sodium sulfate, filtered and concentrated to dryness (1s,4s)-4-(((2-amino-6-chloro-4-methylpyridin-3-yl)methyl)amino)-N-(3-chloro-4-methoxyphenyl)cyclohexanecarboxamide (121 mg, 96% yield) brown solid. LCMS ESI m/z 436.8, 438.8 [M+H]⁺.

### Step 7

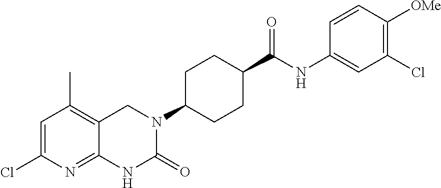 [SMILES: COc1ccc(NC(=O)[C@H]2CC[C@@H](N3Cc4c(C)cc(Cl)nc4NC3=O)CC2)cc1Cl]

### (1s,4s)-N-(3-Chloro-4-methoxyphenyl)-4-(7-chloro-5-methyl-2-oxo-1,2-dihydropyrido[2,3-d]pyrimidin-3(4H)-yl)cyclohexanecarboxamide

To a solution of (1s,4s)-4-(((2-amino-6-chloro-4-methylpyridin-3-yl)methyl)amino)-N-(3-chloro-4-methoxyphenyl)cyclohexanecarboxamide (121 mg, 277 μmol) in DCM (12.9 mL) was added triphosgene (29.3 mg, 96.8 μmol) followed by dropwise addition of a solution of triethylamine (233 μL, 1.66 mmol) in 0.5 mL of DCM. The reaction mixture was stirred at room temperature for 2 h. After complete conversion of the starting material the reaction mixture was concentrated to dryness and the crude residue was purified by flash chromatography on silica gel (25 g column, 1 to 60% (20% MeOH in DCM) in DCM) to yield (1s,4s)-N-(3-Chloro-4-methoxyphenyl)-4-(7-chloro-5-methyl-2-oxo-1,2-dihydropyrido[2,3-d]pyrimidin-3(4H)-yl)cyclohexanecarboxamide (77.1 mg, 60% yield) as a brownish solid. LCMS ESI m/z 462.9, 464.9 [M+H]⁺.

### Step 8

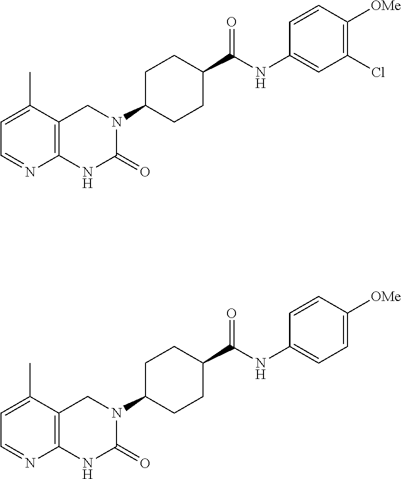 [SMILES: COc1ccc(NC(=O)[C@H]2CC[C@@H](N3Cc4c(C)ccnc4NC3=O)CC2)cc1Cl ; COc1ccc(NC(=O)[C@H]2CC[C@@H](N3Cc4c(C)ccnc4NC3=O)CC2)cc1]

### (1s,4s)-N-(3-Chloro-4-methoxyphenyl)-4-(5-methyl-2-oxo-1,2-dihydropyrido[2,3-d]pyrimidin-3(4H)-yl)cyclohexanecarboxamide and (1s,4s)-N-(4-Methoxyphenyl)-4-(5-methyl-2-oxo-1,2-dihydropyrido[2,3-d]pyrimidin-3(4H)-yl)cyclohexanecarboxamide

A solution of (1s,4s)-N-(3-chloro-4-methoxyphenyl)-4-(7-chloro-5-methyl-2-oxo-1,2-dihydropyrido[2,3-d]pyrimidin-3(4H)-yl)cyclohexanecarboxamide (77.1 mg, 166 μmol) in MeOH (7.74 mL) was purged with nitrogen prior to the addition of 10% Palladium (17.7 mg, 16.6 μmol) on carbon. The reaction flask was purged with hydrogen and the reaction was stirred at room temperature for 16 h. The reaction mixture was purged with nitrogen and was filtered through Celite and concentrated under reduced pressure. Crude residue was purified by reverse phase flash chromatography on C18 (30 g column, 2 to 60% MeCN in water-10 mM Ammonium formate pH=3.8). Pure fractions were directly lyophilized to yield (1s,4s)-N-(3-Chloro-4-methoxyphenyl)-4-(5-methyl-2-oxo-1,2-dihydropyrido[2,3-d]pyrimidin-3(4H)-yl)cyclohexanecarboxamide (Example 14) (11.7 mg, 16% yield) as white solid. LCMS ESI m/z 429.4 [M+H]⁺. ¹H NMR (400 MHz, dmso-d6) δ 9.78 (s, 1H), 9.43 (s, 1H), 7.88 (d, J=4.8 Hz, 1H), 7.83-7.77 (m, 1H), 7.43 (dt, J=8.9, 2.2 Hz, 1H), 7.08 (dd, J=9.1, 1.9 Hz, 1H), 6.73 (d, J=5.0 Hz, 1H), 4.25 (s, 2H), 4.23-4.13 (m, 1H), 3.79 (s, 3H), 2.64-2.59 (m, 1H), 2.16 (s, 3H), 2.14-2.04 (m, 2H), 1.98 (q, J=11.3 Hz, 2H), 1.65 (t, J=12.9 Hz, 2H), 1.45 (d, J=10.9 Hz, 2H); and (1s,4s)-N-(4-methoxyphenyl)-4-(5-methyl-2-oxo-1,2-dihydropyrido[2,3-d]pyrimidin-3(4H)-yl)cyclohexanecarboxamide (Example 15) (15.3 mg, 23% yield) as white solid. LCMS ESI m/z 395.3 [M+H]⁺. ¹H NMR (400 MHz, DMSO-d₆) δ 9.62 (s, 1H), 9.43 (s, 1H), 7.88 (d, J=5.1 Hz, 1H), 7.50 (d, J=9.0 Hz, 2H), 6.85 (d, J=9.0 Hz, 2H), 6.73 (d, J=5.2 Hz, 1H), 4.24 (s, 2H), 4.18 (tt, J=11.9, 3.5 Hz, 1H), 3.69 (s, 3H), 2.64-2.58 (m, 1H), 2.15 (s, 3H), 2.10 (d, J=13.2 Hz, 2H), 2.05-1.93 (m, 2H), 1.69-1.58 (m, 2H), 1.44 (d, J=9.5 Hz, 2H).

### Preparation of Common Intermediate (1s,4s)-4-amino-N-(3-methoxy-4-methylphenyl)cyclohexanecarboxamide

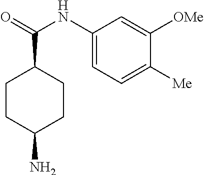 [SMILES: COc1cc(NC(=O)[C@H]2CC[C@@H](N)CC2)ccc1C]

### (1s,4s)-4-amino-N-(3-methoxy-4-methylphenyl)cyclohexanecarboxamide

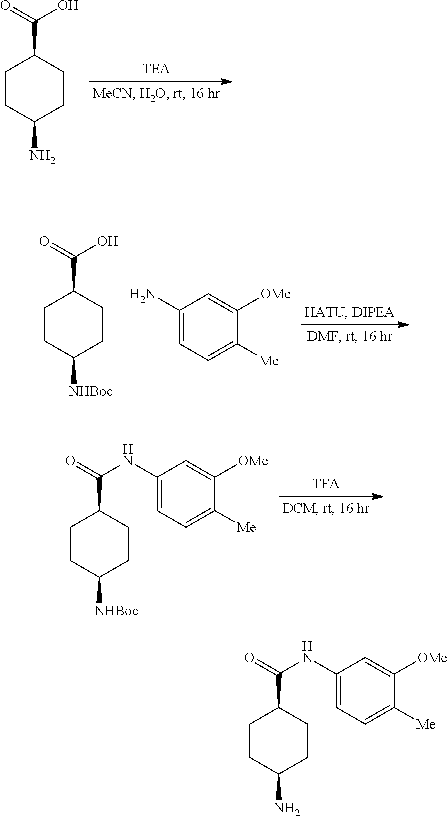 [SMILES: N[C@H]1CC[C@@H](C(=O)O)CC1 ; CC(C)(C)OC(=O)N[C@H]1CC[C@@H](C(=O)O)CC1 ; COc1cc(N)ccc1C ; COc1cc(NC(=O)[C@H]2CC[C@@H](NC(=O)OC(C)(C)C)CC2)ccc1C ; COc1cc(NC(=O)[C@H]2CC[C@@H](N)CC2)ccc1C]

### Step 1

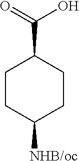

### (1s,4s)-4-(tert-butoxycarbonylamino)cyclohexanecarboxylic Acid

To a solution of 4-aminocyclohexanecarboxylic acid (90 g, 630 mmol) in acetonitrile (900 mL) and water (900 mL) was added tert-butoxycarbonyl tert-butyl carbonate (137 g, 629 mmol) and triethylamine (64 g, 630 mmol, 88 mL). The resulting mixture was stirred at 25° C. for 16 hours. The reaction mixture was concentrated under reduced pressure. The residue was acidified with 1N aqueous hydrochloric acid solution until the solution reached pH 5-6, followed by extraction of the aqueous phase with dichloromethane (200 mL). The organic extract was dried over anhydrous magnesium sulfate, filtered and the filtrate concentrated under reduced pressure to give (1s,4s)-4-(tert-butoxycarbonylamino)cyclohexanecarboxylic acid (120 g, 493 mmol) as a white solid. ¹H NMR (400 MHz, DMSO-d₆) δ 12.02 (s, 1H), 6.72 (d, J=6.2 Hz, 1H), 3.33-3.08 (m, 1H), 2.38-2.36 (m, 1H), 1.88-1.83 (m, 2H), 1.58-1.39 (m, 6H), 1.37 (s, 9H).

### Step 2

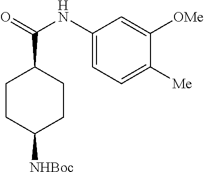 [SMILES: COc1cc(NC(=O)[C@H]2CC[C@@H](NC(=O)OC(C)(C)C)CC2)ccc1C]

### tert-butyl (1s,4s)-4-(3-methoxy-4-methylphenylcarbamoyl)cyclohexylcarbamate

A solution of 4-(tert-butoxycarbonylamino)cyclohexanecarboxylic acid (40 g, 160 mmol) in DMF (200 mL) was added N-ethyl-N-isopropyl-propan-2-amine (63.7 g, 493 mmol, 85.9 mL), N,N,N′,N′-tetramethyl-1-(3-oxido-2,3-dihydrotriazolo[4,5-b]pyridin-3-ium-1-yl)methanediamine; hexafluorophosphate (75.4 g, 197 mmol) and 3-methoxy-4-methyl-aniline (27.1 g, 197 mmol) was stirred at room temperature for 16 hours. The mixture was poured into ethyl acetate (300 mL), the organic layer washed with water (150 mL*3), and then the organic layer dried with Na₂SO₄, filtered and the filtrate concentrated under reduced pressure to give the crude product. Purified product was isolated by silica gel chromatography (5-10% ethyl acetate in hexanes) to give tert-butyl N-[4-[(3-methoxy-4-methyl-phenyl)carbamoyl]cyclohexyl]-carbamate (53.3 g, 147 mmol) as a white solid. LCMS ESI m/z: 307.2 [M+H−56]⁺.

### Step 3

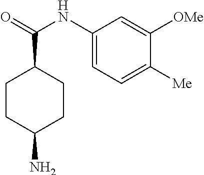 [SMILES: COc1cc(NC(=O)[C@H]2CC[C@@H](N)CC2)ccc1C]

### (1s,4s)-4-amino-N-(3-methoxy-4-methylphenyl)cyclohexanecarboxamide

To a solution of tert-butyl N-[4-[(3-methoxy-4-methyl-phenyl)carbamoyl]cyclohexyl]carbamate (80 g, 220 mmol) in DCM (78 mL) was added 2,2,2-trifluoroacetic acid (33.8 g, 297 mmol, 22.9 mL). The resulting mixture was stirred at 25° C. for 16 hours. The reaction was basified with saturated sodium bicarbonate solution until the solution reached pH 7-8 and then extracted with ethyl acetate (100 ml*3). The organic extracts were dried by Na₂SO₄, filtered and the filtrate concentrated under reduced pressure to give 4-amino-N-(3-methoxy-4-methyl-phenyl)-cyclohexanecarboxamide (50 g, 190 mmol) as an off-white solid. LCMS ESI m/z: 263.2 [M+H]⁺. The product was used in subsequent steps without further purification. ¹H NMR (400 MHz, DMSO-d₆) δ 9.81 (s, 1H), 7.35 (d, J=1.6 Hz, 1H), 7.14-6.94 (m, 2H), 3.74 (s, 3H), 3.28-3.08 (m, 1H), 2.54-2.52 (m, 1H), 2.08 (s, 3H), 1.98-1.88 (m, 2H), 1.86-1.56 (m, 6H).

### Example 16

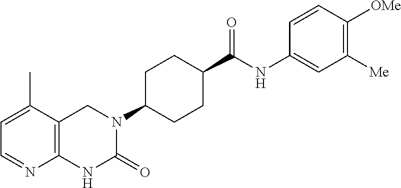 [SMILES: COc1ccc(NC(=O)[C@H]2CC[C@@H](N3Cc4c(C)ccnc4NC3=O)CC2)cc1C]

### (1s,4s)-N-(4-Methoxy-3-methylphenyl)-4-(5-methyl-2-oxo-1,2-dihydropyrido[2,3-d]pyrimidin-3(4H)-yl)cyclohexanecarboxamide

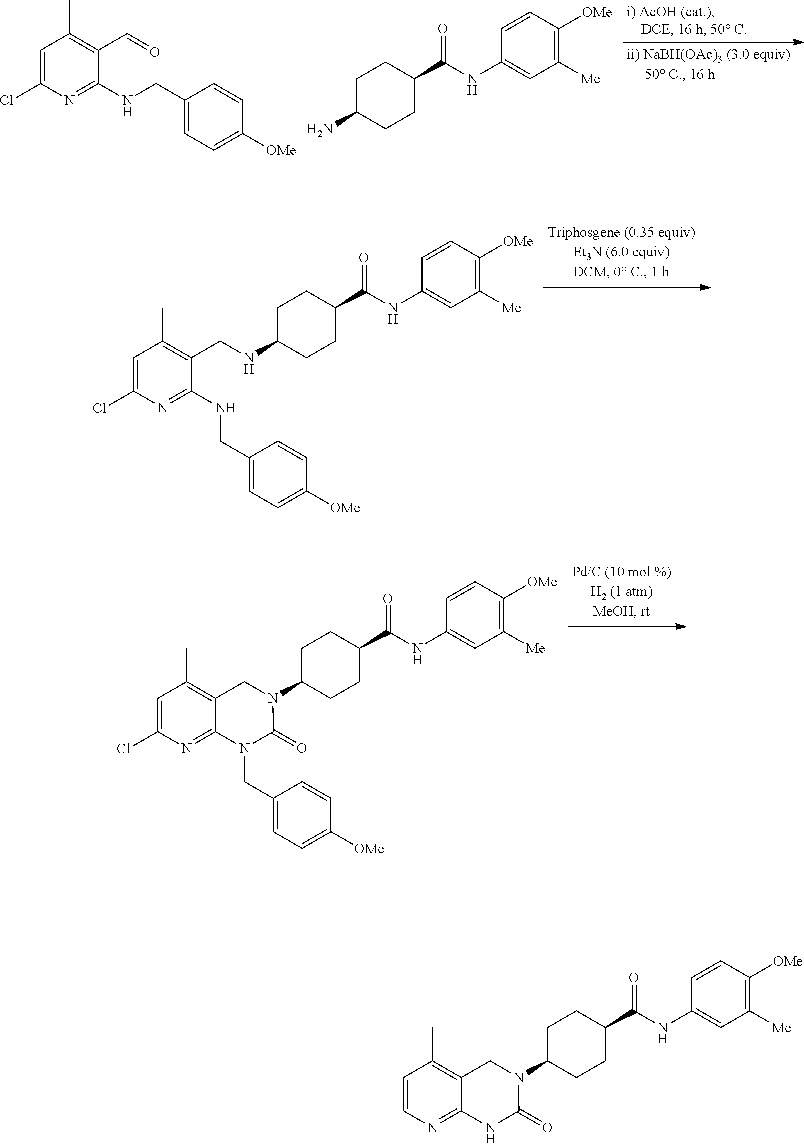 [SMILES: COc1ccc(CNc2nc(Cl)cc(C)c2C=O)cc1 ; COc1ccc(NC(=O)[C@H]2CC[C@@H](N)CC2)cc1C ; COc1ccc(CNc2nc(Cl)cc(C)c2CN[C@H]2CC[C@@H](C(=O)Nc3ccc(OC)c(C)c3)CC2)cc1 ; COc1ccc(CN2C(=O)N([C@H]3CC[C@@H](C(=O)Nc4ccc(OC)c(C)c4)CC3)Cc3c(C)cc(Cl)nc32)cc1 ; COc1ccc(NC(=O)[C@H]2CC[C@@H](N3Cc4c(C)ccnc4NC3=O)CC2)cc1C]

### Step 1

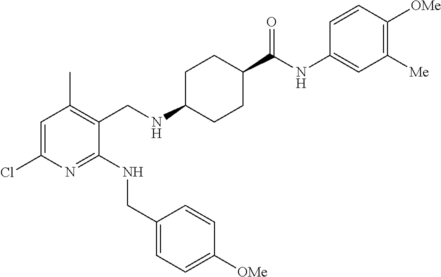 [SMILES: COc1ccc(CNc2nc(Cl)cc(C)c2CN[C@H]2CC[C@@H](C(=O)Nc3ccc(OC)c(C)c3)CC2)cc1]

### (1s,4s)-4-(((6-Chloro-2-((4-methoxybenzyl)amino)-4-methylpyridin-3-yl)methyl)amino)-N-(4-methoxy-3-methylphenyl)cyclohexanecarboxamide

6-Chloro-2-((4-methoxybenzyl)amino)-4-methylnicotinaldehyde (100 mg, 344 μmol) and (1s,4s)-4-amino-N-(3-methoxy-4-methylphenyl)cyclohexanecarboxamide (90.2 mg, 344 μmol) were place in a vial and DCE (2.29 mL) was added followed by a drop of acetic acid. The reagents were mixed for 16 hour at 50° C. Sodium triacetoxyborohydride (218 mg, 1.03 mmol) was added in one portion. The reaction mixture was stirred at 50° C. for 16 h. MeOH was added to the reaction mixture and the resulting solution was stirred at room temperature for 1h. Concentrated to dryness. The crude residue was purified by flash chromatography on silica gel (from 1 to 60% (20% MeOH in DCM) in DCM) to yield (1s,4s)-4-(((6-Chloro-2-((4-methoxybenzyl)amino)-4-methylpyridin-3-yl)methyl)amino)-N-(4-methoxy-3-methylphenyl)cyclohexanecarboxamide (114 mg, 62% yield) as a purple solid. LCMS ESI m/z 537.2 [M+H]⁺.

### Step 2

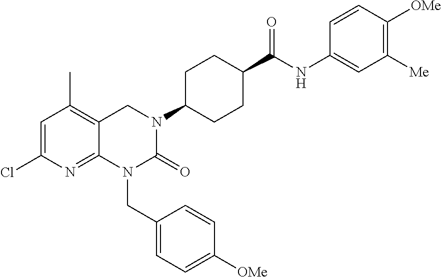 [SMILES: COc1ccc(CN2C(=O)N([C@H]3CC[C@@H](C(=O)Nc4ccc(OC)c(C)c4)CC3)Cc3c(C)cc(Cl)nc32)cc1]

### (1s,4s)-4-(7-Chloro-1-(4-methoxybenzyl)-5-methyl-2-oxo-1,2-dihydropyrido[2,3-d]pyrimidin-3(4H)-yl)-N-(4-methoxy-3-methylphenyl)cyclohexanecarboxamide

To a solution of (1s,4s)-4-(((6-chloro-2-((4-methoxybenzyl)amino)-4-methylpyridin-3-yl)methyl)amino)-N-(4-methoxy-3-methylphenyl)cyclohexanecarboxamide (105 mg, 195 μmol) in DCM (9.10 mL) was added triphosgene (20.7 mg, 68.4 μmol) followed by dropwise addition of a solution of triethylamine (164 uL, 1.17 mmol) in 0.5 mL of DCM. The reaction mixture was concentrated to dryness and the crude residue was purified by flash chromatography on silica gel (25 g column, 1 to 60% (20% MeOH in DCM) in DCM) to yield (1s,4s)-4-(7-Chloro-1-(4-methoxybenzyl)-5-methyl-2-oxo-1,2-dihydropyrido[2,3-d]pyrimidin-3(4H)-yl)-N-(4-methoxy-3-methylphenyl)cyclohexanecarboxamide (35.0 mg, 32% yield) as a brownish solid. LCMS ESI m/z 564.1 [M+H]⁺.

### Step 3

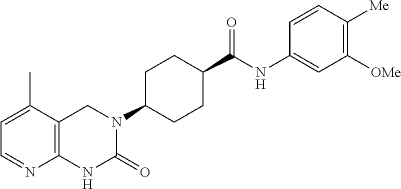 [SMILES: COc1cc(NC(=O)[C@H]2CC[C@@H](N3Cc4c(C)ccnc4NC3=O)CC2)ccc1C]

### (1s,4s)-N-(4-Methoxy-3-methylphenyl)-4-(5-methyl-2-oxo-1,2-dihydropyrido[2,3-d]pyrimidin-3(4H)-yl)cyclohexanecarboxamide

To a solution of (1s,4s)-4-(7-Chloro-1-(4-methoxybenzyl)-5-methyl-2-oxo-1,2-dihydropyrido[2,3-d]pyrimidin-3(4H)-yl)-N-(4-methoxy-3 methylphenyl)cyclohexanecarboxamide (35.0 mg, 62.2 μmol) in MeOH (3.50 mL) placed under nitrogen was added palladium (7.01 mg, 6.59 μmol) (Pd 10% wt/C) and the reaction mixture was then purged with hydrogen and stirred under hydrogen atmosphere (1 atm) at room temperature for 1 day. The reaction mixture was purged with nitrogen and filtered through Celite. Concentrated under reduced pressure. Crude residue was purified by reverse phase flash chromatography on C18 (30 g column, 2 to 60% MeCN in water-10 mM Ammonium formate pH=3.8). Pure fractions were directly lyophilized to yield (1s,4s)-N-(4-Methoxy-3-methylphenyl)-4-(5-methyl-2-oxo-1,2-dihydropyrido[2,3-d]pyrimidin-3(4H)-yl)cyclohexanecarboxamide (2.89 mg, 11% yield) as a white solid. LCMS ESI m/z 409.3 [M+H]⁺. ¹H NMR (400 MHz, DMSO-d₆) δ 9.54 (s, 1H), 9.44 (s, 1H), 7.88 (d, J=5.1 Hz, 1H), 7.36 (s, 1H), 7.37-7.33 (m, 1H), 6.83 (d, J=9.1 Hz, 1H), 6.73 (d, J=5.3 Hz, 1H), 4.24 (s, 2H), 4.22-4.14 (m, 1H), 3.72 (s, 3H), 2.63-2.57 (m, 1H), 2.15 (s, 3H), 2.10 (s, 3H), 2.08-2.06 (m, 2H), 2.00 (q, J=11.8 Hz, 2H), 1.70-1.55 (m, 2H), 1.43 (dd, J=11.8, 2.4 Hz, 2H).

### Example 17

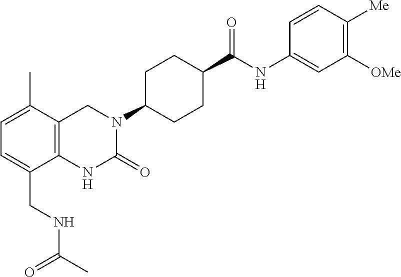 [SMILES: COc1cc(NC(=O)[C@H]2CC[C@@H](N3Cc4c(C)ccc(CNC(C)=O)c4NC3=O)CC2)ccc1C]

### (1s,4s)-4-(8-(Acetamidomethyl)-5-methyl-2-oxo-1,2-dihydroquinazolin-3(4H)-yl)-N-(3-methoxy-4-methylphenyl)cyclohexanecarboxamide

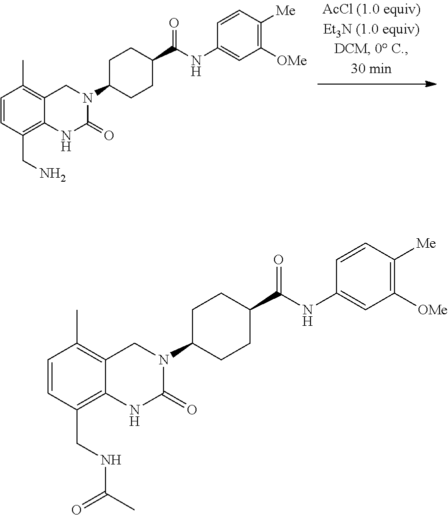 [SMILES: COc1cc(NC(=O)[C@H]2CC[C@@H](N3Cc4c(C)ccc(CN)c4NC3=O)CC2)ccc1C ; COc1cc(NC(=O)[C@H]2CC[C@@H](N3Cc4c(C)ccc(CNC(C)=O)c4NC3=O)CC2)ccc1C]

### Step 1

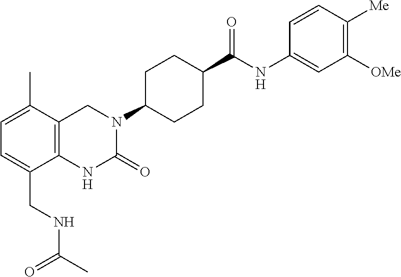 [SMILES: COc1cc(NC(=O)[C@H]2CC[C@@H](N3Cc4c(C)ccc(CNC(C)=O)c4NC3=O)CC2)ccc1C]

### (1s,4s)-4-(8-(Acetamidomethyl)-5-methyl-2-oxo-1,2-dihydroquinazolin-3(4H)-yl)-N-(3-methoxy-4-methylphenyl)cyclohexanecarboxamide

To a vial with (1,4s)-4-(8-(aminomethyl)-5-methyl-2-oxo-1,2-dihydroquinazolin-3(4H)-yl)-N-(3-methoxy-4-methylphenyl)cyclohexanecarboxamide from Example 58 (64.1 mg, 103 μmol) in DCM (1.03 mL) and triethylamine (14.3 μL, 103 μmol) at 0° C. was added acetyl chloride (7.46 μL, 103 μmol). After 30 minutes at 0° C., the crude reaction mixture was concentrated under reduced pressure and purified using reverse phase column (10 g, C18, using MeCN:AMF (10 mM) 0% to 80% yield). The desired product fractions were combined and was lyophilized yielding desired product (12.4 mg, 25% yield) as a white powder. LCMS ESI m/z 479.4 [M+H]⁺. ¹H NMR (400 MHz, DMSO-d₆) δ 9.68 (s, 1H), 8.58 (s, 1H), 8.44 (t, J=6.2 Hz, 1H), 7.26 (d, J=1.6 Hz, 1H), 7.08 (dd, J=8.0, 1.7 Hz, 1H), 6.99 (d, J=8.1 Hz, 1H), 6.92 (d, J=7.7 Hz, 1H), 6.66 (d, J=7.8 Hz, 1H), 4.17 (s, 2H), 4.16-4.11 (m, 1H), 4.09 (d, J=6.2 Hz, 2H), 3.72 (s, 3H), 2.62 (s, 1H), 2.12 (s, 4H), 2.06 (d, J=6.3 Hz, 4H), 2.04-1.91 (m, 2H), 1.81 (s, 3H), 1.63 (t, J=13.2 Hz, 2H), 1.43 (d, J=9.3 Hz, 2H).

### Example 18

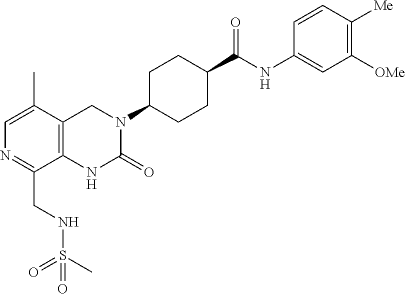 [SMILES: COc1cc(NC(=O)[C@H]2CC[C@@H](N3Cc4c(C)cnc(CNS(C)(=O)=O)c4NC3=O)CC2)ccc1C]

### (1s,4s)-N-(3-Methoxy-4-methylphenyl)-4-(5-methyl-8-(methylsulfonamidomethyl)-2-oxo-1,2-dihydropyrido[3,4-d]pyrimidin-3(4H)-yl)cyclohexanecarboxamideoxamide

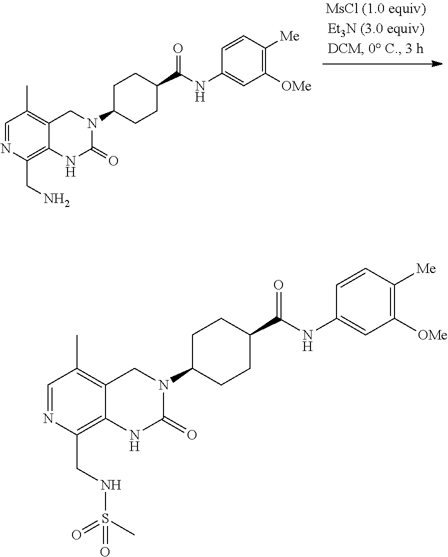 [SMILES: COc1cc(NC(=O)[C@H]2CC[C@@H](N3Cc4c(C)cnc(CN)c4NC3=O)CC2)ccc1C ; COc1cc(NC(=O)[C@H]2CC[C@@H](N3Cc4c(C)cnc(CNS(C)(=O)=O)c4NC3=O)CC2)ccc1C]

### Step 1

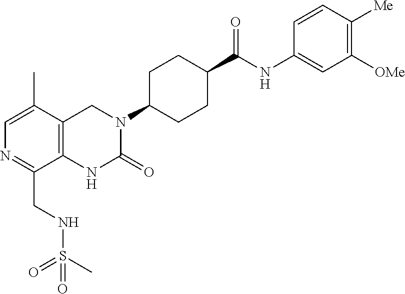 [SMILES: COc1cc(NC(=O)[C@H]2CC[C@@H](N3Cc4c(C)cnc(CNS(C)(=O)=O)c4NC3=O)CC2)ccc1C]

### (1s,4s)-N-(3-Methoxy-4-methylphenyl)-4-(5-methyl-8-(methylsulfonamidomethyl)-2-oxo-1,2-dihydropyrido[3,4-d]pyrimidin-3(4H)-yl)cyclohexanecarboxamideoxamide

To a solution of (1s,4s)-4-(8-(aminomethyl)-5-methyl-2-oxo-1,2-dihydropyrido[3,4-d]pyrimidin-3(4H)-yl)-N-(3-methoxy-4-methylphenyl)cyclohexanecarboxamide from Example 20 (26.0 mg, 59.4 μmol) in DCM (432 μL) was added triethylamine (9.06 μL, 178 μmol) and methanesulfonyl chloride (4.69 μL, 59.4 μmol) and the reaction mixture was stirred at 0° C. for 3 h. The reaction mixture was concentrated to dryness and directly loaded on a C18 column and purified by reverse phase flash chromatography (30 g, 2 to 60% MeCN in water-10 mM Ammonium formate pH=3.8). Fractions were directly lyophilized to yield (1s,4s)-N-(3-Methoxy-4-methylphenyl)-4-(5-methyl-8-(methylsulfonamidomethyl)-2-oxo-1,2-dihydropyrido[3,4-d]pyrimidin-3(4H)-yl)cyclohexanecarboxamideoxamide (3.78 mg, 12% yield) as a white solid. LCMS ESI m/z 516.3 [M+H]⁺. ¹H NMR (400 MHz, DMSO-d₆) δ 9.69 (s, 1H), 8.38 (s, 1H), 8.14 (s, 1H), 7.88 (s, 1H), 7.32 (t, J=5.9 Hz, 1H), 7.26 (d, J=1.7 Hz, 1H), 7.08 (dd, J=8.0, 1.8 Hz, 1H), 6.99 (d, J=8.2 Hz, 1H), 4.26 (s, 2H), 4.20 (d, J=6.1 Hz, 2H), 4.17-4.13 (m, 1H), 3.72 (s, 3H), 2.88 (s, 3H), 2.65-2.60 (m, 1H), 2.13 (s, 3H), 2.11-2.06 (m, 2H), 2.05 (s, 3H), 2.03-1.91 (m, 2H), 1.70-1.57 (m, 2H), 1.44 (dd, J=11.6, 1.7 Hz, 2H).

### Example 19

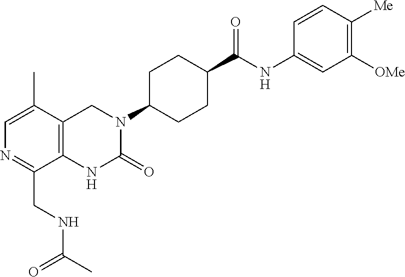 [SMILES: COc1cc(NC(=O)[C@H]2CC[C@@H](N3Cc4c(C)cnc(CNC(C)=O)c4NC3=O)CC2)ccc1C]

### (1s,4s)-4-(8-(Acetamidomethyl)-5-methyl-2-oxo-1,2-dihydropyrido[3,4-d]pyrimidin-3(4H)-yl)-N-(3-methoxy-4-methylphenyl)cyclohexanecarboxamide

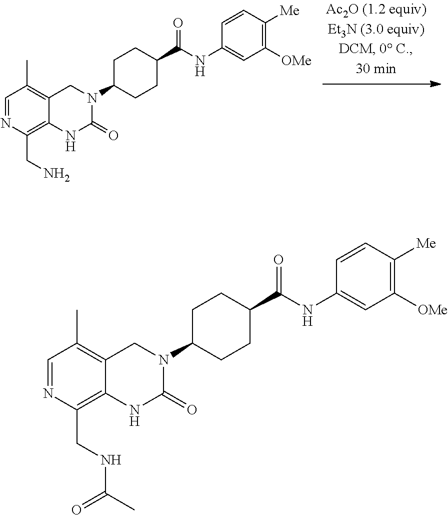 [SMILES: COc1cc(NC(=O)[C@H]2CC[C@@H](N3Cc4c(C)cnc(CN)c4NC3=O)CC2)ccc1C ; COc1cc(NC(=O)[C@H]2CC[C@@H](N3Cc4c(C)cnc(CNC(C)=O)c4NC3=O)CC2)ccc1C]

### Step 1

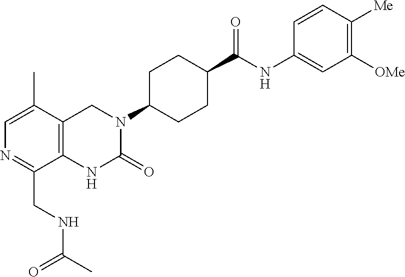 [SMILES: COc1cc(NC(=O)[C@H]2CC[C@@H](N3Cc4c(C)cnc(CNC(C)=O)c4NC3=O)CC2)ccc1C]

### (1s,4s)-4-(8-(Acetamidomethyl)-5-methyl-2-oxo-1,2-dihydroquinazolin-3(4H)-yl)-N-(3-methoxy-4-methylphenyl)cyclohexanecarboxamide

To a solution of (1s,4s)-4-(8-(aminomethyl)-5-methyl-2-oxo-1,2-dihydropyrido[3,4-d]pyrimidin-3(4H)-yl)-N-(3-methoxy-4-methylphenyl)cyclohexanecarboxamide from Example 20 (26.0 mg, 59.4 μmol) in DCM (432 μL) was added triethylamine (9.06 μL, 178 μmol) and acetic anhydride (6.29 μL, 66.6 μmol) and the reaction mixture was stirred at room temperature for 3 h. The reaction mixture was concentrated to dryness and directly loaded on a C18 column and purified by reverse phase flash chromatography (30 g, 2 to 60% MeCN in water-10 mM Ammonium formate pH=3.8). Fractions were directly lyophilized to yield (1s,4s)-4-(8-(Acetamidomethyl)-5-methyl-2-oxo-1,2-dihydroquinazolin-3(4H)-yl)-N-(3-methoxy-4-methylphenyl)cyclohexanecarboxamide (8.27 mg, 29% yield) as a white solid. LCMS ESI m/z 480.3 [M+H]⁺. ¹H NMR (400 MHz, dmso-d6) δ 9.68 (s, 1H), 8.82 (s, 1H), 8.68 (t, J=6.2 Hz, 1H), 7.84 (s, 1H), 7.26 (d, J=1.6 Hz, 1H), 7.08 (dd, J=8.1, 1.7 Hz, 1H), 6.99 (d, J=8.1 Hz, 1H), 4.23 (s, 2H), 4.22 (d, J=6.6 Hz, 2H), 4.19-4.10 (m, 1H), 3.72 (s, 3H), 2.65-2.59 (m, 1H), 2.11 (s, 3H), 2.10-2.06 (m, 2H), 2.05 (s, 3H), 2.02-1.89 (m, 2H), 1.82 (s, 3H), 1.69-1.56 (m, 2H), 1.47-1.37 (m, 2H).

### Example 20

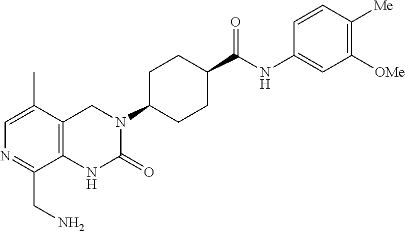 [SMILES: COc1cc(NC(=O)[C@H]2CC[C@@H](N3Cc4c(C)cnc(CN)c4NC3=O)CC2)ccc1C]

### (1s,4s)-4-(8-(Aminomethyl)-5-methyl-2-oxo-1,2-dihydropyrido[3,4-d]pyrimidin-3(4H)-yl)-N-(3-methoxy-4-methylphenyl)cyclohexanecarboxamide

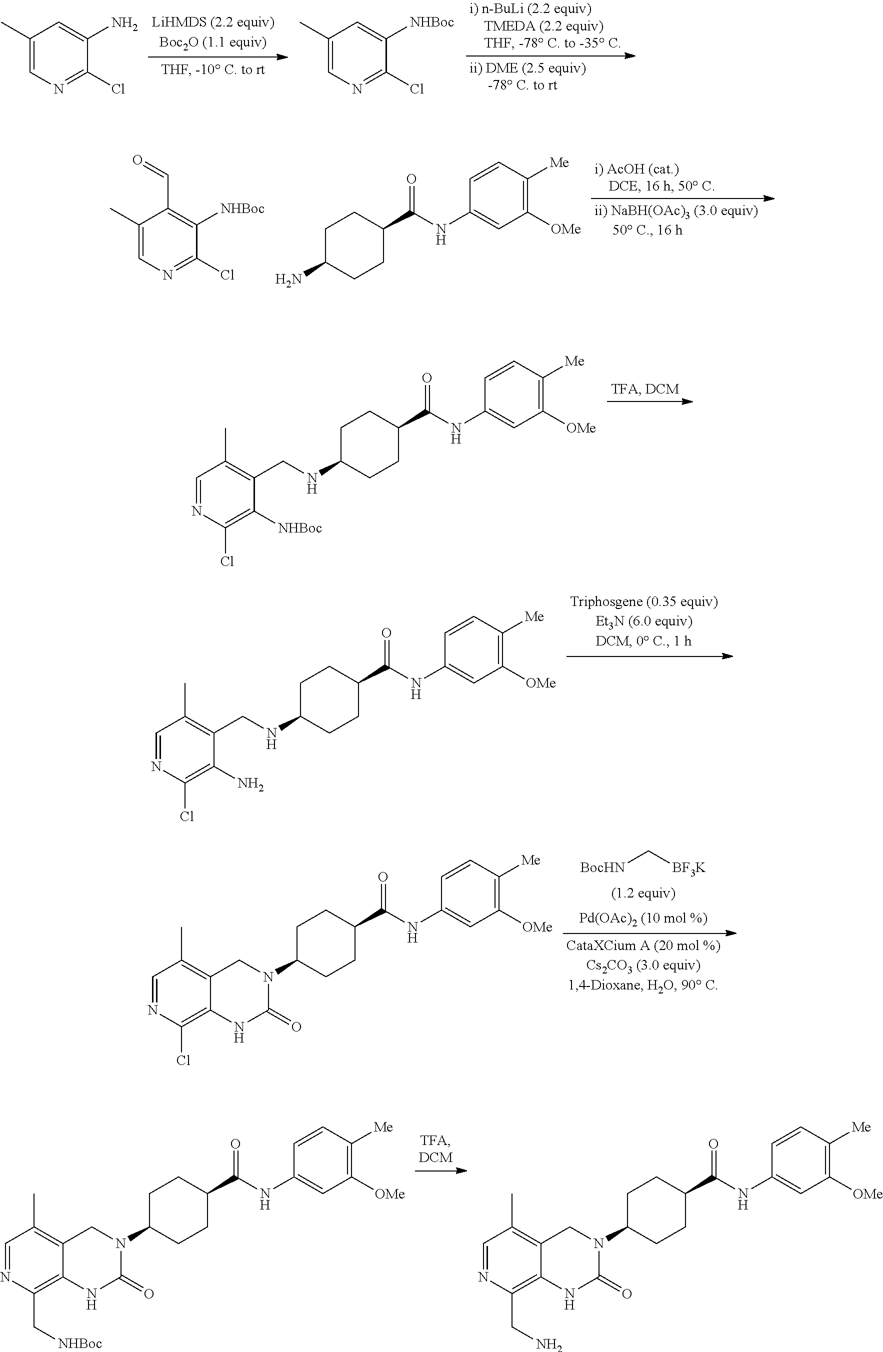

### Step 1

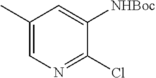 [SMILES: Cc1cnc(Cl)c(NC(=O)OC(C)(C)C)c1]

### tert-Butyl (2-chloro-5-methylpyridin-3-yl)carbamate

To a −10° C. solution of 3-amino-2-chloro-5-methylpyridine (1.50 g, 10.3 mmol) in THF (25.9 mL) was added dropwise lithium bis(trimethylsilyl)amide solution in THF (22.8 mL, 22.8 mmol) and the reaction mixture was stirred for 30 minutes after which, a solution of di-tert-butyl dicarbonate (2.61 mL, 11.4 mmol) in THF (25.9 mL) was added dropwise. The reaction mixture was stirred at −10° C. for another 30 minutes and allowed to warm to room temperature. Upon completion of the reaction, a saturated solution of ammonium chloride was added and the product was extracted three time with EtOAc. Organics were combined, washed with water, brine, dried with Na₂SO₄, filtered and concentrated to yield a crude yellow oil. It was purified by flash chromatography on silica gel (40 g column, 0 to 60% EtOAc in heptanes) to yield tert-Butyl(2-chloro-5-methylpyridin-3-yl)carbamate (200 mg, 8% yield) as a yellow oil. LCMS ESI m/z 242.9 [M+H]⁺.

### Step 2

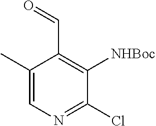 [SMILES: Cc1cnc(Cl)c(NC(=O)OC(C)(C)C)c1C=O]

### tert-Butyl(2-chloro-4-formyl-5-methylpyridin-3-yl)carbamate

Into a 50 mL round-bottomed flask, purged and maintained with an inert atmosphere of nitrogen, was placed a solution of tert-butyl(2-chloro-5-methylpyridin-3-yl)carbamate (400 mg, 1.65 mmol) in THF (8.24 mL) and N,N,N′,N′-tetramethylethylenediamine (546 μL, 3.63 mmol) (TMEDA). The vessel was cooled to −78° C. and n-butyllithium (1.44 mL, 3.61 mmol) was added dropwise. The resulting solution was stirred for 30 min at −35° C. The resulting solution was cooled to −78° C. and dimethylformamide (320 μL, 4.12 mmol) was added dropwise with stirring. After 30 min, at −78° C. the reaction mixture was placed at −35° C. and slowly allowed to warm to room temperature. A saturated solution of ammonium chloride was added to reaction mixture and it was extracted 3 times with EtOAc. Organics were combined, washed with brine, dried with anhydrous sodium sulfate, filtered and concentrated to dryness. The crude residue was purified by flash chromatography on silica gel (30 g column, 0 to 70% EtOAc in heptanes) to yield tert-Butyl(2-chloro-4-formyl-5-methylpyridin-3-yl)carbamate (163 mg, 37% yield) as a yellow solid. LCMS ESI m/z 214.0 [M-tBu]⁺.

### Step 3

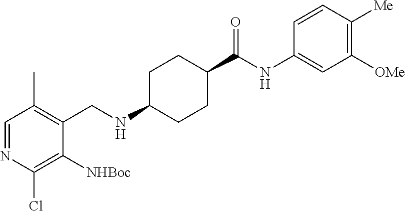 [SMILES: COc1cc(NC(=O)[C@H]2CC[C@@H](NCc3c(C)cnc(Cl)c3NC(=O)OC(C)(C)C)CC2)ccc1C]

### tert-butyl(2-chloro-4-((((1s,4s)-4-((3-methoxy-4-methylphenyl)carbamoyl)cyclohexyl)amino)methyl)-5-methylpyridin-3-yl)carbamate

tert-Butyl(2-chloro-4-formyl-5-methylpyridin-3-yl)carbamate (163 mg, 602 μmol) and (1s,4s)-4-amino-N-(3-methoxy-4-methylphenyl)cyclohexanecarboxamide (160 mg, 608 μmol) were place in a vial with DCE (4.01 mL) and a drop of acetic acid. The reagents were mixed for 16 hours at 50° C. Reaction mixture was cooled to room temperature and sodium triacetoxyborohydride (381 mg, 1.81 mmol) was added in one portion. The reaction mixture was stirred at 23° C. for 3 h. MeOH was added to the reaction mixture and the resulting solution was stirred at room temperature for 30 min. Concentrated to dryness. The crude residue was purified by flash chromatography on silica gel (25 g column, 1 to 60% (20% MeOH in DCM) in DCM) to yield tert-butyl(2-chloro-4-((((1s,4s)-4-((3-methoxy-4-methylphenyl)carbamoyl)cyclohexyl)amino)methyl)-5-methylpyridin-3-yl)carbamate (270 mg, 87% yield) as a beige solid. LCMS ESI m/z 517.0 [M+H]⁺.

### Step 4

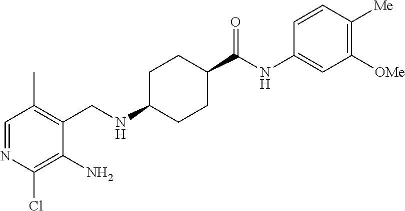 [SMILES: COc1cc(NC(=O)[C@H]2CC[C@@H](NCc3c(C)cnc(Cl)c3N)CC2)ccc1C]

### (1s,4s)-4-(((3-Amino-2-chloro-5-methylpyridin-4-yl)methyl)amino)-N-(3-methoxy-4-methylphenyl)cyclohexanecarboxamide

To a solution of (1s,4s)-N-(3-methoxy-4-methylphenyl)-4-(((3-((4-methoxybenzyl)amino)-5-methylpyridin-4-yl)methyl)amino)cyclohexanecarboxamide (250 mg, 484 μmol) in DCM was added trifluoroacetic acid (37.0 μL, 484 μmol) and the reaction mixture was stirred at room temperature for 1 h. The reaction mixture was concentrated to dryness and a saturated solution of sodium bicarbonate was added to the crude residue and it was extracted 3 times with EtOAc. Organics were combined, washed with brine, dried with anhydrous sodium sulfate, filtered and concentrated to dryness to yield (1s,4s)-4-(((3-Amino-2-chloro-5-methylpyridin-4-yl)methyl)amino)-N-(3-methoxy-4-methylphenyl)cyclohexanecarboxamide (224 mg, quant.) as an off white solid. LCMS ESI m/z 417.2 [M+H]⁺.

### Step 5

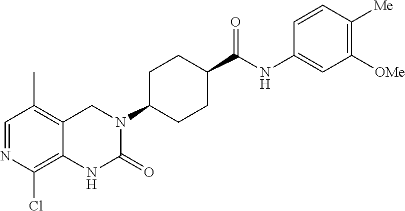 [SMILES: COc1cc(NC(=O)[C@H]2CC[C@@H](N3Cc4c(C)cnc(Cl)c4NC3=O)CC2)ccc1C]

### (1s,4s)-4-(8-Chloro-5-methyl-2-oxo-1,2-dihydropyrido[3,4-d]pyrimidin-3(4H)-yl)-N-(3-methoxy-4-methylphenyl)cyclohexanecarboxamide

To a suspension of (1s,4s)-4-(((3-amino-2-chloro-5-methylpyridin-4-yl)methyl)amino)-N-(3-methoxy-4-methylphenyl)cyclohexanecarboxamide (201 mg, 482 μmol) in DCM (22.4 mL) was added triphosgene (51.1 mg, 169 μmol) followed by dropwise addition of a solution of triethylamine (405 μL, 2.89 mmol) in 0.5 mL of DCM. The reaction mixture was stirred at room temperature for 2 h. After complete conversion of the starting material the reaction mixture was concentrated to dryness and the crude residue was purified by reverse phase flash chromatography on C18 (30 g column, 0 to 70% MeCN in water-10 mM Ammonium formate pH=3.8). Pure fractions were directly lyophilized to yield (1s,4s)-4-(8-Chloro-5-methyl-2-oxo-1,2-dihydropyrido[3,4-d]pyrimidin-3(4H)-yl)-N-(3-methoxy-4-methylphenyl)cyclohexanecarboxamide (198 mg, 93% yield) as a white solid. LCMS ESI m/z 443.0 [M+H]⁺. ¹H NMR (400 MHz, DMSO-d₆) δ 9.67 (s, 1H), 8.60 (s, 1H), 7.76 (s, 1H), 7.28 (d, J=1.6 Hz, 1H), 7.09 (dd, J=8.0, 1.7 Hz, 1H), 7.00 (d, J=8.3 Hz, 1H), 4.31 (s, 2H), 4.19 (tt, J=11.9, 3.6 Hz, 1H), 3.73 (s, 3H), 2.68-2.59 (m, 1H), 2.14 (s, 3H), 2.13-2.08 (m, 2H), 2.06 (s, 3H), 1.99 (qd, J=12.5, 3.3 Hz, 2H), 1.73-1.60 (m, 2H), 1.51-1.41 (m, 2H).

### Step 6

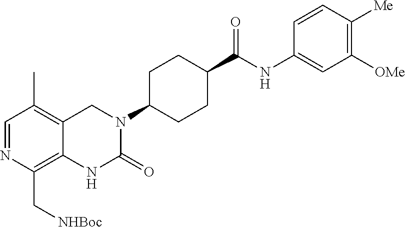 [SMILES: COc1cc(NC(=O)[C@H]2CC[C@@H](N3Cc4c(C)cnc(CNC(=O)OC(C)(C)C)c4NC3=O)CC2)ccc1C]

### tert-Butyl ((3-((1s,4s)-4-((3-methoxy-4-methylphenyl)carbamoyl)cyclohexyl)-5-methyl-2-oxo-1,2,3,4-tetrahydropyrido[3,4-d]pyrimidin-8-yl)methyl)carbamate

A microwave vial was charged with, potassium (((tert-butoxycarbonyl)amino)methyl)trifluoroborate (13.2 mg, 54.2 μmol), (1s,4s)-4-(8-chloro-5-methyl-2-oxo-1,2-dihydropyrido[3,4-d]pyrimidin-3(4H)-yl)-N-(3-methoxy-4-methylphenyl)cyclohexanecarboxamide (20.0 mg, 45.2 μmol) and cesium carbonate (44.1 mg, 135 μmol). Water (75.3 μL) and 1,4-dioxane (376 μL) were added, the vial was capped with a septum and the reaction mixture was degassed with a nitrogen flow for 10 minutes. Finally, palladium (II) Acetate (1.01 mg, 4.52 μmol) and butyldi-1-adamantylphosphine (CataXCium A) (3.41 mg, 9.03 μmol) were then added to the vial under nitrogen and the reaction mixture was capped and stirred at 90° C. for 16 h. The reaction mixture was cold to room temperature and the reaction mixture filtered through a short silica gel pad. Concentration to dryness provided with crude desired product that was used directly for next step tert-Butyl ((3-((1s,4s)-4-((3-methoxy-4-methylphenyl)carbamoyl)cyclohexyl)-5-methyl-2-oxo-1,2,3,4-tetrahydropyrido[3,4-d]pyrimidin-8-yl)methyl)carbamate (24.5 mg, 101% yield) as an off white solid. LCMS ESI m/z 538.4 [M+H]⁺.

### Step 7

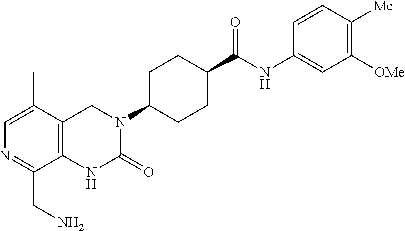 [SMILES: COc1cc(NC(=O)[C@H]2CC[C@@H](N3Cc4c(C)cnc(CN)c4NC3=O)CC2)ccc1C]

### (1s,4s)-4-(8-(Aminomethyl)-5-methyl-2-oxo-1,2-dihydropyrido[3,4-d]pyrimidin-3(4H)-yl)-N-(3-methoxy-4-methylphenyl)cyclohexanecarboxamide

To a solution of (1s,4s)-4-(8-(Aminomethyl)-5-methyl-2-oxo-1,2-dihydropyrido[3,4-d]pyrimidin-3(4H)-yl)-N-(3-methoxy-4-methylphenyl)cyclohexanecarboxamide (20.0 mg, 37.2 μmol) in DCM (200 μL) was added trifluoroacetic acid (200.0 μL, 2.61 mmol) and the reaction mixture was stirred at room temperature for 1 h. The reaction was concentrated to dryness and directly purified by reverse phase flash chromatography on C18 (30 g column, 2 to 60% MeCN in water-10 mM Ammonium formate pH=3.8). Pure fractions were directly lyophilized to yield (1s,4s)-4-(8-(Aminomethyl)-5-methyl-2-oxo-1,2-dihydropyrido[3,4-d]pyrimidin-3(4H)-yl)-N-(3-methoxy-4-methylphenyl)cyclohexanecarboxamide (13.1 mg, 80% yield) as a white solid. LCMS ESI m/z 438.3 [M+H]⁺. ¹H NMR (400 MHz, DMSO-d₆) δ 9.72 (s, 1H), 8.27 (s, 1H), 7.89 (s, 1H), 7.29 (d, J=1.7 Hz, 1H), 7.12 (dd, J=8.0, 1.9 Hz, 1H), 7.02 (d, J=8.1 Hz, 1H), 4.29 (s, 2H), 4.26-4.16 (m, 1H), 3.94 (s, 2H), 3.75 (s, 3H), 2.66-2.61 (m, 1H), 2.15 (s, 3H), 2.14-2.08 (m, 2H), 2.08 (s, 3H), 2.06-1.94 (m, 2H), 1.72-1.60 (m, 2H), 1.50-1.41 (m, 2H).

### Example 21

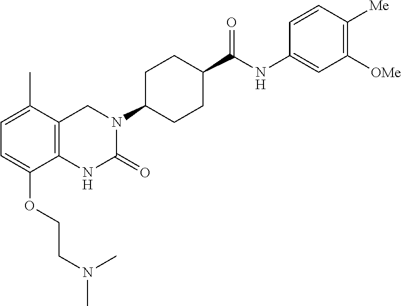 [SMILES: COc1cc(NC(=O)[C@H]2CC[C@@H](N3Cc4c(C)ccc(OCCN(C)C)c4NC3=O)CC2)ccc1C]

### (1s,4s)-4-(8-(2-(Dimethylamino)ethoxy)-5-methyl-2-oxo-1,2-dihydroquinazolin-3(4H)-yl)-N-(3-methoxy-4-methylphenyl)cyclohexanecarboxamide

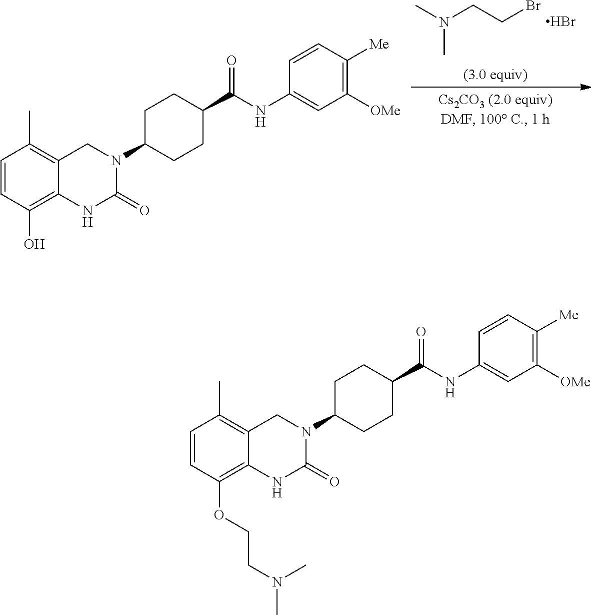

### Step 1

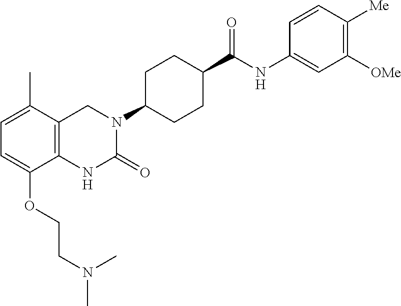 [SMILES: COc1cc(NC(=O)[C@H]2CC[C@@H](N3Cc4c(C)ccc(OCCN(C)C)c4NC3=O)CC2)ccc1C]

### (1s,4s)-4-(8-(2-(Dimethylamino)ethoxy)-5-methyl-2-oxo-1,2-dihydroquinazolin-3(4H)-yl)-N-(3-methoxy-4-methylphenyl)cyclohexanecarboxamide

To a flask with (1s,4s)-4-(8-hydroxy-5-methyl-2-oxo-1,2-dihydroquinazolin-3(4H)-yl)-N-(3-methoxy-4-methylphenyl)cyclohexanecarboxamide from Example 61 (25.0 mg, 44.9 μmol) was added DMF (4.49 mL) and Cs₂CO₃ (29.5 mg, 89.7 μmol). The mixture was stirred at r.t. for 2 min and then 2-bromo-N,N-dimethylethanamine hydrobromide (31.4 mg, 135 μmol) was added and the reaction mixture was heated to 100° C. for 1 hour. After reaction completion sat. NaHCO₃ was added to the reaction mixture and it was extracted with EtOAc (×3), washed with brine, dried over Na₂SO₄ and concentrated under reduced pressure. The crude residue was purified using reverse phase column (10 g, C18) using 0 to 80% MeCN:AMF (10 mM). The product was concentrated and lyophilized. The final product was isolated as a pale yellow solid (11.7 mg, 52% yield). LCMS ESI m/z 495.4 [M+H]⁺. ¹H NMR (400 MHz, DMSO-d₆) δ 9.72 (s, 1H), 8.38-8.27 (m, 1H), 7.29 (s, 1H), 7.12 (d, J=8.0 Hz, 1H), 7.02 (d, J=8.1 Hz, 1H), 6.80 (d, J=8.2 Hz, 1H), 6.66 (d, J=8.3 Hz, 1H), 4.23 (s, 2H), 4.23-4.15 (m, 1H), 3.97 (t, J=5.4 Hz, 2H), 3.75 (s, 3H), 2.65 (s, J=0.8 Hz, 1H), 2.59 (t, J=5.4 Hz, 2H), 2.26-2.19 (m, 6H), 2.17-2.07 (m, 8H), 2.06-1.93 (m, 2H), 1.75-1.59 (m, 2H), 1.52-1.39 (m, 2H).

### Example 22

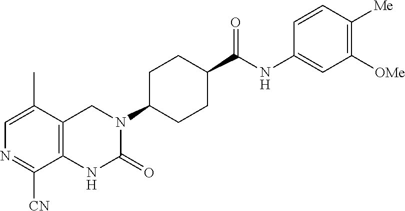 [SMILES: COc1cc(NC(=O)[C@H]2CC[C@@H](N3Cc4c(C)cnc(C#N)c4NC3=O)CC2)ccc1C]

### (1s,4s)-4-(8-Cyano-5-methyl-2-oxo-1,2-dihydropyrido[3,4-d]pyrimidin-3(4H)-yl)-N-(3-methoxy-4-methylphenyl)cyclohexanecarboxamide

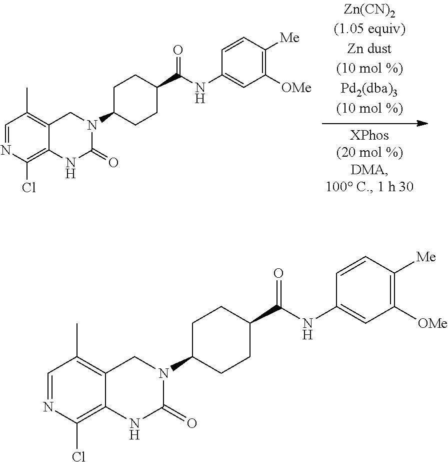 [SMILES: COc1cc(NC(=O)[C@H]2CC[C@@H](N3Cc4c(C)cnc(Cl)c4NC3=O)CC2)ccc1C ; COc1cc(NC(=O)[C@H]2CC[C@@H](N3Cc4c(C)cnc(Cl)c4NC3=O)CC2)ccc1C]

### Step 1

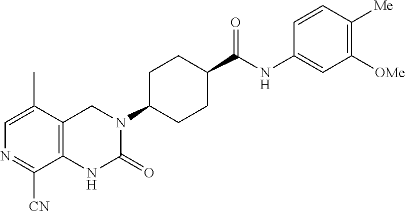 [SMILES: COc1cc(NC(=O)[C@H]2CC[C@@H](N3Cc4c(C)cnc(C#N)c4NC3=O)CC2)ccc1C]

### (1s,4s)-4-(8-Cyano-5-methyl-2-oxo-1,2-dihydropyrido[3,4-d]pyrimidin-3(4H)-yl)-N-(3-methoxy-4-methylphenyl)cyclohexanecarboxamide

A microwave vial was loaded with 2-dicyclohexylphosphino-2′,4′,6′-triisopropylbiphenyl (22.2 mg, 45.2 μmol), tris(dibenzylideneacetone)-dipalladium (0) (20.7 mg, 22.6 μmol), zinc (1.48 mg, 22.6 μmol), zinc cyanide (27.8 mg, 237 μmol), (1s,4s)-4-(8-chloro-5-methyl-2-oxo-1,2-dihydropyrido[3,4-d]pyrimidin-3(4H)-yl)-N-(3-methoxy-4-methylphenyl)cyclohexanecarboxamide (100 mg, 226 μmol) and was capped and purged with nitrogen using vacuum and nitrogen backfilling (3 times). In the In the meantime, DMA (4.52 mL) was degassed using a stream of nitrogen bubbled in the solvent for 15 minutes. DMA (4.52 mL) was added to the reagents and the reaction mixture was heated and stirred at 100° C. for 90 min. The reaction mixture was cooled to room temperature and filtered through Celite. The filtrate was diluted with a saturated solution of sodium bicarbonate and it was extracted 3 times with EtOAc. Organics were combined, washed with water, brine, dried with anhydrous sodium sulfate, filtered and concentrated to dryness. The crude residue was purified by flash chromatography on silica gel (25 g column, 20 to 80% EtOAc in DCM) to yield (1s,4s)-4-(8-cyano-5-methyl-2-oxo-1,2-dihydropyrido[3,4-d]pyrimidin-3(4H)-yl)-N-(3-methoxy-4-methylphenyl)cyclohexanecarboxamide (64.8 mg, 66% yield) as an off white solid. LCMS ESI m/z 434.1 [M+H]⁺. ¹H NMR (400 MHz, DMSO-D6) δ 9.70 (s, 1H), 9.51 (s, 1H), 8.07 (s, 1H), 7.28 (d, J=1.6 Hz, 1H), 7.09 (dd, J=8.1, 1.8 Hz, 1H), 7.00 (d, J=8.4 Hz, 1H), 4.33 (s, 2H), 4.16 (tt, J=12.2, 3.9 Hz, 1H), 3.73 (s, 3H), 2.67-2.62 (m, 1H), 2.22 (s, 3H), 2.11 (d, J=13.3 Hz, 2H), 2.06 (s, 3H), 1.98 (qd, J=12.5, 1.7 Hz, 2H), 1.71-1.59 (m, J=13.0 Hz, 2H), 1.46 (dd, J=11.9, 2.6 Hz, 2H).

### Example 23

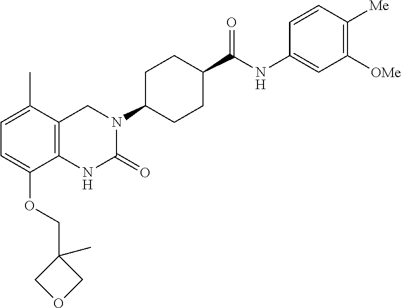 [SMILES: COc1cc(NC(=O)[C@H]2CC[C@@H](N3Cc4c(C)ccc(OCC5(C)COC5)c4NC3=O)CC2)ccc1C]

### (1s,4s)-N-(3-Methoxy-4-methylphenyl)-4-(5-methyl-8-((3-methyloxetan-3-yl)methoxy)-2-oxo-1,2-dihydroquinazolin-3(4H)-yl)cyclohexanecarboxamide

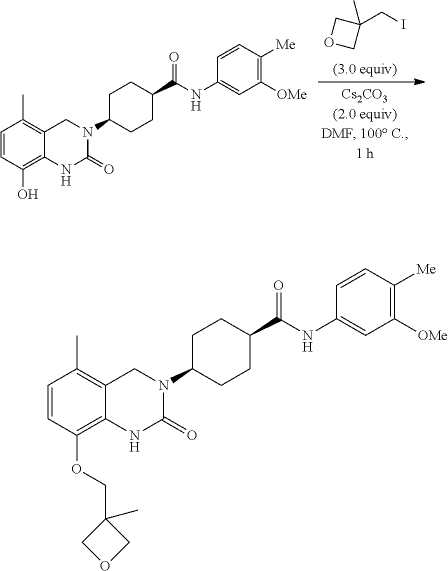

### Step 1

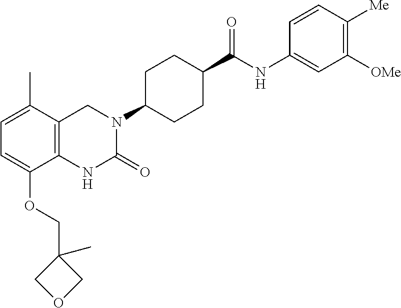 [SMILES: COc1cc(NC(=O)[C@H]2CC[C@@H](N3Cc4c(C)ccc(OCC5(C)COC5)c4NC3=O)CC2)ccc1C]

### (1s,4s)-N-(3-Methoxy-4-methylphenyl)-4-(5-methyl-8-((3-methyloxetan-3-yl)methoxy)-2-oxo-1,2-dihydroquinazolin-3(4H)-yl)cyclohexanecarboxamide

To a flask with (1s,4s)-4-(8-hydroxy-5-methyl-2-oxo-1,2-dihydroquinazolin-3(4H)-yl)-N-(3-methoxy-4-methylphenyl)cyclohexanecarboxamide from Example 61 (25.0 mg, 44.9 μmol) was added Cs₂CO₃ (29.5 mg, 89.7 μmol) and DMF (449 uL). To the mix after 2 min of stirring at r.t. was added 3-(iodomethyl)-3-methyloxetane (28.5 mg, 135 μmol). The reaction was then heated to 100° C. for 1 h. After 1 hour, the reaction was quenched with sat. NaHCO₃ and extracted with EtOAc (×3), washed with brine, dried over Na₂SO₄ and concentrated under reduced pressure to yield the crude residue. The crude residue was then purified using reverse phase column (10 g C18, 0 to 80% MeCN:AMF (10 mM). After lyophilization, the product was repurified using normal phase column (10 g, 0 to 20% MeOH:DCM). The product was lyophilized to yield (1s,4s)-N-(3-methoxy-4-methylphenyl)-4-(5-methyl-8-((3-methyloxetan-3-yl)methoxy)-2-oxo-1,2-dihydroquinazolin-3(4H)-yl)cyclohexanecarboxamide (0.9 mg, 4% yield) as a white powder. LCMS ESI m/z 434.1 [M+H]⁺. ¹H NMR (400 MHz, DMSO-d₆) δ 9.71 (s, 1H), 7.79 (s, 1H), 7.29 (s, 1H), 7.10 (s, 1H), 7.02 (d, J=8.0 Hz, 1H), 6.79 (s, 1H), 6.69 (d, J=7.4 Hz, 1H), 4.46 (d, J=5.8 Hz, 2H), 4.32 (d, J=5.2 Hz, 2H), 4.23 (s, 2H), 4.03 (s, 2H), 3.75 (s, 3H), 2.12 (s, 5H), 2.08 (s, 4H), 2.04-1.98 (m, 2H), 1.67 (s, 2H), 1.48 (s, 2H), 1.37 (s, 3H), 1.23 (s, 2H).

### Example 24

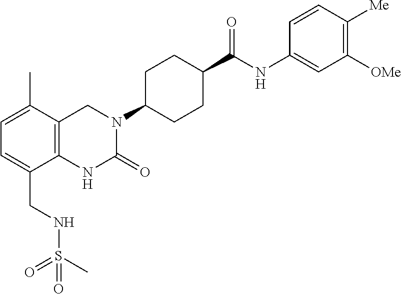 [SMILES: COc1cc(NC(=O)[C@H]2CC[C@@H](N3Cc4c(C)ccc(CNS(C)(=O)=O)c4NC3=O)CC2)ccc1C]

### (1s,4s)-N-(3-Methoxy-4-methylphenyl)-4-(5-methyl-8-(methylsulfonamidomethyl)-2-oxo-1,2-dihydroquinazolin-3(4H)-yl)cyclohexanecarboxamide

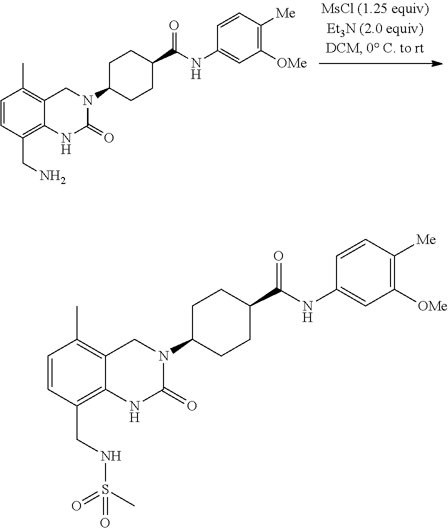 [SMILES: COc1cc(NC(=O)[C@H]2CC[C@@H](N3Cc4c(C)ccc(CN)c4NC3=O)CC2)ccc1C ; COc1cc(NC(=O)[C@H]2CC[C@@H](N3Cc4c(C)ccc(CNS(C)(=O)=O)c4NC3=O)CC2)ccc1C]

### Step 1

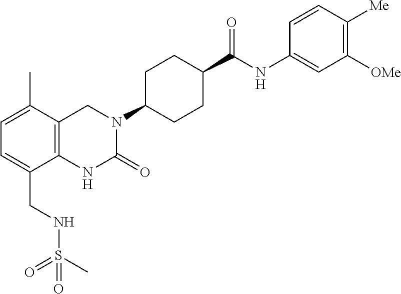 [SMILES: COc1cc(NC(=O)[C@H]2CC[C@@H](N3Cc4c(C)ccc(CNS(C)(=O)=O)c4NC3=O)CC2)ccc1C]

### (1s,4s)-N-(3-Methoxy-4-methylphenyl)-4-(5-methyl-8-(methylsulfonamidomethyl)-2-oxo-1,2-dihydroquinazolin-3(4H)-yl)cyclohexanecarboxamide

To a flask with (1s,4s)-4-(8-(aminomethyl)-5-methyl-2-oxo-1,2-dihydroquinazolin-3(4H)-yl)-N-(3-methoxy-4-methylphenyl)cyclohexanecarboxamide from Example 58 (30.0 mg, 68.7 μmol) was added DCM (687 uL) and NEt₃ (19.3 uL, 137 μmol). The mixture was cooled down to 0° C., then methanesulfonyl chloride (6.78 uL, 85.9 μmol) was added dropwise and stirred for 15 min. The reaction mixture was stirred at r.t. for 1 h. After reaction completion sat. NaHCO₃ was added to the reaction mixture and extracted with EtOAc (×3), washed with brine, dried over Na₂SO₄ and concentrated under reduced pressure. The crude residue was purified using a reverse phase column (10 g, C18) using 0 to 100% MeCN:AMF (10 mM). The product was the concentrated and lyophilized. The product was repurified using normal phase column (10 g, 0 to 20% MeOH: DCM). The product was concentrated and lyophilized. (1s,4s)-N-(3-Methoxy-4-methylphenyl)-4-(5-methyl-8-(methylsulfonamidomethyl)-2-oxo-1,2-dihydroquinazolin-3(4H)-yl)cyclohexanecarboxamide was isolated in (2.91 mg, 8.2% yield). LCMS ESI m/z 515.4 [M+H]⁺. ¹H NMR (400 MHz, DMSO-d₆) δ 9.70 (s, 1H), 8.09 (s, 1H), 7.38 (t, J=6.3 Hz, 1H), 7.28 (s, 1H), 7.09 (s, 1H), 7.01 (d, J=7.9 Hz, 2H), 6.72 (d, J=8.0 Hz, 1H), 4.22 (s, 2H), 4.21-4.14 (m, 1H), 4.12-4.03 (m, 2H), 3.74 (s, 3H), 2.90 (s, 2H), 2.18-2.12 (m, 4H), 2.12-2.06 (m, 5H), 2.06-1.96 (m, 3H), 1.73-1.60 (m, 2H), 1.50-1.41 (m, 2H)

### Example 25

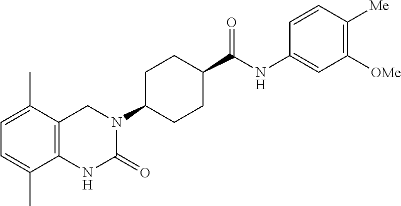 [SMILES: COc1cc(NC(=O)[C@H]2CC[C@@H](N3Cc4c(C)ccc(C)c4NC3=O)CC2)ccc1C]

### (1s,4s)-4-(5,8-Dimethyl-2-oxo-1,2-dihydroquinazolin-3(4H)-yl)-N-(3-methoxy-4-methylphenyl)cyclohexanecarboxamide

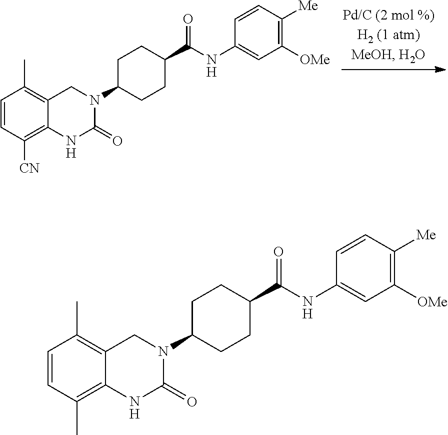 [SMILES: COc1cc(NC(=O)[C@H]2CC[C@@H](N3Cc4c(C)ccc(C#N)c4NC3=O)CC2)ccc1C ; COc1cc(NC(=O)[C@H]2CC[C@@H](N3Cc4c(C)ccc(C)c4NC3=O)CC2)ccc1C]

### Step 1

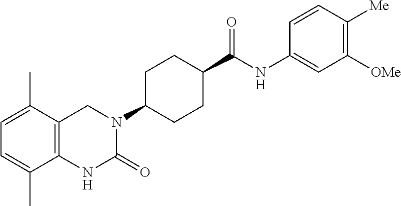 [SMILES: COc1cc(NC(=O)[C@H]2CC[C@@H](N3Cc4c(C)ccc(C)c4NC3=O)CC2)ccc1C]

### (1s,4s)-4-(5,8-Dimethyl-2-oxo-1,2-dihydroquinazolin-3(4H)-yl)-N-(3-methoxy-4-methylphenyl)cyclohexanecarboxamide

To a flask with (1s,4s)-4-(8-cyano-5-methyl-2-oxo-1,2-dihydroquinazolin-3(4H)-yl)-N-(3-methoxy-4-methylphenyl)cyclohexanecarboxamide from Example 59 (18.2 mg, 42.1 μmol) and palladium on carbon 10% loading (896 mg). The flask was then purged with N₂ (g) for 15 minutes then MeOH (553 μL) and water (111 μL) were added under nitrogen. The reaction was then placed under hydrogen atmosphere using a balloon and the mixture was directly sparged for 5 minutes before leaving it under a H₂(g) atmosphere overnight. After overnight stirring the reaction mixture was flushed with nitrogen before workup. The reaction mixture was diluted with EtOAc and MeOH and filtered over celite and concentrated under reduced pressure to yield the crude mixture. The product was then purified using reverse phase column chromatography (C18, 10 g 0 to 80% MeCN:AMF (10 mM)). (1s,4s)-4-(5,8-Dimethyl-2-oxo-1,2-dihydroquinazolin-3(4H)-yl)-N-(3-methoxy-4-methylphenyl)cyclohexanecarboxamide was isolated (5.93 mg, 33% yield) as a pale yellow solid after lyophilization. LCMS ESI m/z 422.3 [M+H]⁺. ¹H NMR (400 MHz, DMSO-d₆) δ 9.72 (s, 1H), 8.27 (s, 1H), 7.29 (s, 1H), 7.17-7.08 (m, 1H), 7.02 (d, J=8.1 Hz, 1H), 6.85 (d, J=7.6 Hz, 1H), 6.62 (d, J=7.6 Hz, 1H), 4.26-4.21 (m, 1H), 4.20 (s, 2H), 3.75 (s, 3H), 2.69-2.61 (m, 1H), 2.18-2.07 (m, 11H), 2.07-1.92 (m, 2H), 1.75-1.59 (m, 2H), 1.53-1.40 (m, 2H)

### Example 26

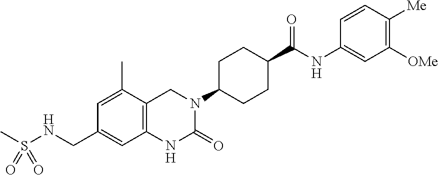 [SMILES: COc1cc(NC(=O)[C@H]2CC[C@@H](N3Cc4c(C)cc(CNS(C)(=O)=O)cc4NC3=O)CC2)ccc1C]

### (1s,4s)-N-(3-Methoxy-4-methylphenyl)-4-(5-methyl-7-(methylsulfonamidomethyl)-2-oxo-1,2-dihydroquinazolin-3(4H)-yl)cyclohexanecarboxamide

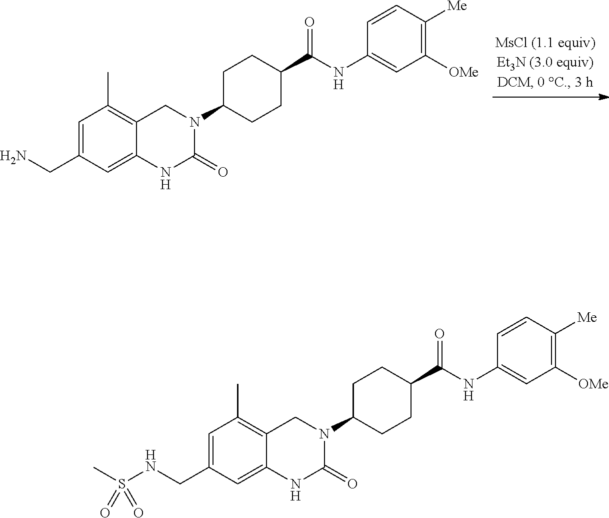 [SMILES: COc1cc(NC(=O)[C@H]2CC[C@@H](N3Cc4c(C)cc(CN)cc4NC3=O)CC2)ccc1C ; COc1cc(NC(=O)[C@H]2CC[C@@H](N3Cc4c(C)cc(CNS(C)(=O)=O)cc4NC3=O)CC2)ccc1C]

### Step 1

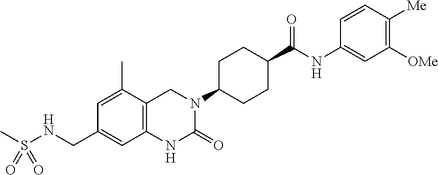 [SMILES: COc1cc(NC(=O)[C@H]2CC[C@@H](N3Cc4c(C)cc(CNS(C)(=O)=O)cc4NC3=O)CC2)ccc1C]

### (1s,4s)-N-(3-Methoxy-4-methylphenyl)-4-(5-methyl-7-(methylsulfonamidomethyl)-2-oxo-1,2-dihydroquinazolin-3(4H)-yl)cyclohexanecarboxamide

To a solution of ((1s,4s)-4-(7-(aminomethyl)-5-methyl-2-oxo-1,2-dihydroquinazolin-3(4H)-yl)-N-(3-methoxy-4-methylphenyl)cyclohexanecarboxamide from Example 28 (30.0 mg, 68.7 μmol) in DCM (500 uL) was added triethylamine (28.9 μL, 206 μmol) and methanesulfonyl chloride (5.97 μL, 75.6 μmol) and the reaction mixture was stirred at room temperature for 3 h. The reaction mixture was concentrated to dryness and directly loaded on a C18 column and purified by reverse phase flash chromatography (30 g column, 2 to 60% MeCN in water-10 mM ammonium formate pH=3.8). The product was lyophilized to yield (1s,4s)-N-(3-Methoxy-4-methylphenyl)-4-(5-methyl-7-(methylsulfonamidomethyl)-2-oxo-1,2-dihydroquinazolin-3(4H)-yl)cyclohexanecarboxamide (6.15 mg, 17% yield) as an off white solid. LCMS ESI m/z 515.3 [M+H]⁺. ¹H NMR (400 MHz, DMSO-d₆) δ 9.66 (s, 1H), 9.10 (s, 1H), 7.43 (t, J=6.3 Hz, 1H), 7.27 (d, J=1.7 Hz, 1H), 7.09 (dd, J=8.1, 1.8 Hz, 1H), 7.00 (d, J=8.4 Hz, 1H), 6.64 (s, 1H), 6.55 (s, 1H), 4.18 (s, 2H), 4.24-4.11 (m, 1H), 3.96 (d, J=6.2 Hz, 2H), 3.73 (s, 3H), 2.83 (s, 3H), 2.64-2.60 (m, 1H), 2.13 (s, 3H), 2.11-2.07 (m, 2H), 2.06 (s, 3H), 1.98 (qd, J=12.5, 3.2 Hz, 2H), 1.69-1.57 (m, 2H), 1.43 (dd, J=11.6, 2.4 Hz, 2H).

### Example 27

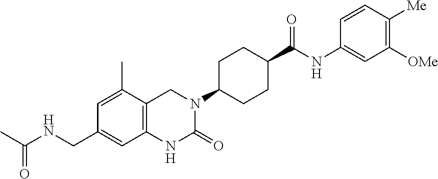 [SMILES: COc1cc(NC(=O)[C@H]2CC[C@@H](N3Cc4c(C)cc(CNC(C)=O)cc4NC3=O)CC2)ccc1C]

### (1s,4s)-4-(7-(Acetamidomethyl)-5-methyl-2-oxo-1,2-dihydroquinazolin-3(4H)-yl)-N-(3-methoxy-4-methylphenyl)cyclohexanecarboxamide

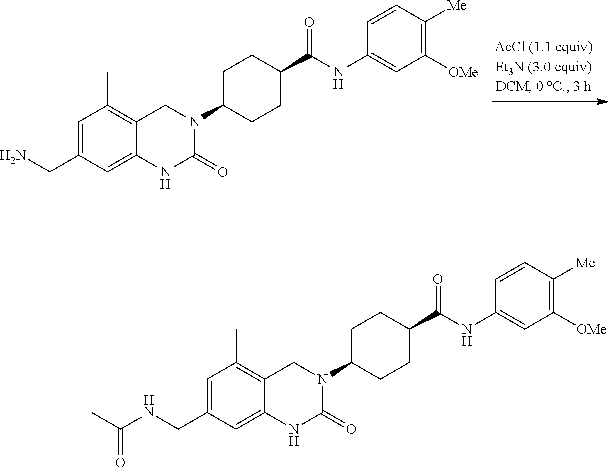 [SMILES: COc1cc(NC(=O)[C@H]2CC[C@@H](N3Cc4c(C)cc(CN)cc4NC3=O)CC2)ccc1C ; COc1cc(NC(=O)[C@H]2CC[C@@H](N3Cc4c(C)cc(CNC(C)=O)cc4NC3=O)CC2)ccc1C]

### Step 1

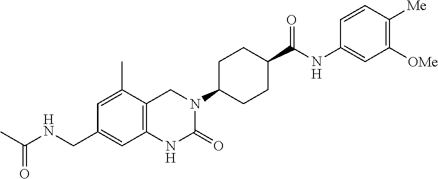 [SMILES: COc1cc(NC(=O)[C@H]2CC[C@@H](N3Cc4c(C)cc(CNC(C)=O)cc4NC3=O)CC2)ccc1C]

### (1s,4s)-4-(7-(Acetamidomethyl)-5-methyl-2-oxo-1,2-dihydroquinazolin-3(4H)-yl)-N-(3-methoxy-4-methylphenyl)cyclohexanecarboxamide

To a solution of ((1s,4s)-4-(7-(aminomethyl)-5-methyl-2-oxo-1,2-dihydroquinazolin-3(4H)-yl)-N-(3-methoxy-4-methylphenyl)cyclohexanecarboxamide from Example 28 (30.0 mg, 68.7 μmol) in DCM (500 uL) was added triethylamine (28.9 μL, 206 μmol) and acetyl chloride (5.48 μL, 75.6 μmol) and the reaction mixture was stirred at room temperature for 3 h. The reaction mixture was concentrated to dryness and directly loaded on a C18 column and purified by reverse phase flash chromatography (30 g column, 2 to 60% MeCN in water-10 mM Ammonium formate pH=3.8). The product was directly lyophilized to yield (1s,4s)-4-(7-(acetamidomethyl)-5-methyl-2-oxo-1,2-dihydroquinazolin-3(4H)-yl)-N-(3-methoxy-4-methylphenyl)cyclohexanecarboxamide (8.28 mg, 25% yield) as a white solid. LCMS ESI m/z 479.4 [M+H]⁺. ¹H NMR (400 MHz, DMSO-d₆) δ 9.66 (s, 1H), 9.03 (s, 1H), 8.21 (t, J=5.8 Hz, 1H), 7.27 (d, J=1.7 Hz, 1H), 7.09 (dd, J=8.0, 1.8 Hz, 1H), 7.00 (d, J=8.0 Hz, 1H), 6.56 (s, 1H), 6.46 (s, 1H), 4.22-4.13 (m, 1H), 4.17 (s, 2H), 4.05 (d, J=5.9 Hz, 2H), 3.73 (s, 3H), 2.66-2.59 (m, 1H), 2.11 (s, 3H), 2.10-2.07 (m, 2H), 2.06 (s, 3H), 1.98 (qd, J=12.7, 3.5 Hz, 2H), 1.81 (s, 3H), 1.69-1.57 (m, 2H), 1.47-1.37 (m, 2H).

### Example 28

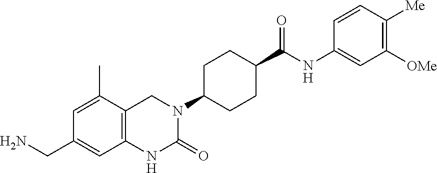 [SMILES: COc1cc(NC(=O)[C@H]2CC[C@@H](N3Cc4c(C)cc(CN)cc4NC3=O)CC2)ccc1C]

### (1s,4s)-4-(7-(Aminomethyl)-5-methyl-2-oxo-1,2-dihydroquinazolin-3(4H)-yl)-N-(3-methoxy-4-methylphenyl)cyclohexanecarboxamide

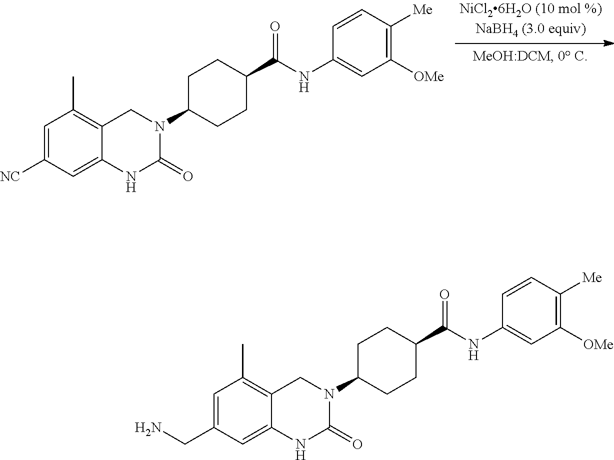 [SMILES: COc1cc(NC(=O)[C@H]2CC[C@@H](N3Cc4c(C)cc(C#N)cc4NC3=O)CC2)ccc1C ; COc1cc(NC(=O)[C@H]2CC[C@@H](N3Cc4c(C)cc(CN)cc4NC3=O)CC2)ccc1C]

### Step 1

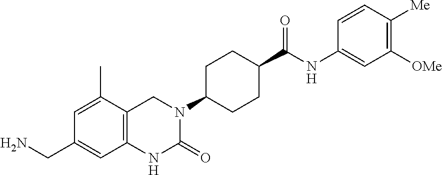 [SMILES: COc1cc(NC(=O)[C@H]2CC[C@@H](N3Cc4c(C)cc(CN)cc4NC3=O)CC2)ccc1C]

### (1s,4s)-4-(7-(Aminomethyl)-5-methyl-2-oxo-1,2-dihydroquinazolin-3(4H)-yl)-N-(3-methoxy-4-methylphenyl)cyclohexanecarboxamide

To a cold 0° C. solution of (1s,4s)-4-(7-cyano-5-methyl-2-oxo-1,2-dihydroquinazolin-3(4H)-yl)-N-(3-methoxy-4-methylphenyl)cyclohexanecarboxamide from Example 31 (20.0 mg, 46.2 μmol) in DCM (567 uL) and MeOH (567 μL) (1:1 mixture) was added Nickel (II) chloride hexahydrate (1.13 mg, 4.76 μmol) followed by slow portion wise addition of sodium borohydride (5.28 uL, 139 μmol). The reaction mixture was stirred at 0° C. for 1 h and slowly turned black. A saturated solution of sodium bicarbonate was added to reaction mixture and it was extracted 3 times with DCM. Organics were combined, washed with brine, dried with anhydrous sodium sulfate, filtered and concentrated to dryness. Crude residue was purified by reverse phase flash chromatography on C18 (30 g column, 2 to 60% MeCN in water-10 mM ammonium formate pH=3.8). The product was directly lyophilized to yield (1s,4s)-4-(7-(aminomethyl)-5-methyl-2-oxo-1,2-dihydroquinazolin-3(4H)-yl)-N-(3-methoxy-4-methylphenyl)cyclohexanecarboxamide (8.52 mg, 42% yield) as white solid. LCMS ESI m/z 437.4 [M+H]⁺. ¹H NMR (400 MHz, DMSO-d₆) δ 9.70 (s, 1H), 9.13 (s, 1H), 8.32 (s, 1H), 7.29 (s, 1H), 7.12 (d, J=7.3 Hz, 1H), 7.02 (d, J=8.1 Hz, 1H), 6.71 (s, 1H), 6.57 (s, 1H), 4.21 (s, 2H), 4.24-4.16 (m, 1H), 3.75 (s, 3H), 3.67 (s, 2H), 2.68-2.65 (m, 1H), 2.15 (s, 3H), 2.13-2.09 (m, 2H), 2.08 (s, 3H), 2.01 (dd, J=23.5, 12.3 Hz, 2H), 1.71-1.59 (m, 2H), 1.45 (d, J=10.0 Hz, 2H).

### Example 29

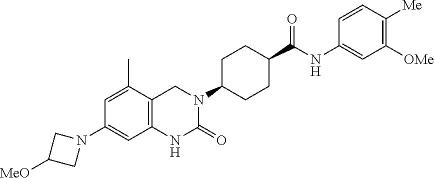 [SMILES: COc1cc(NC(=O)[C@H]2CC[C@@H](N3Cc4c(C)cc(N5CC(OC)C5)cc4NC3=O)CC2)ccc1C]

### (1s,4s)-N-(3-Methoxy-4-methylphenyl)-4-(7-(3-methoxyazetidin-1-yl)-5-methyl-2-oxo-1,2-dihydroquinazolin-3(4H)-yl)cyclohexanecarboxamide

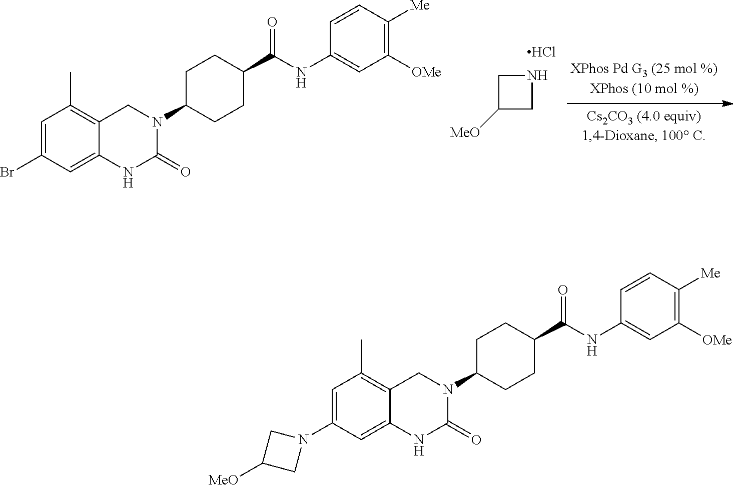 [SMILES: COc1cc(NC(=O)[C@H]2CC[C@@H](N3Cc4c(C)cc(Br)cc4NC3=O)CC2)ccc1C ; COC1CNC1 ; Cl ; COc1cc(NC(=O)[C@H]2CC[C@@H](N3Cc4c(C)cc(N5CC(OC)C5)cc4NC3=O)CC2)ccc1C]

### Step 1

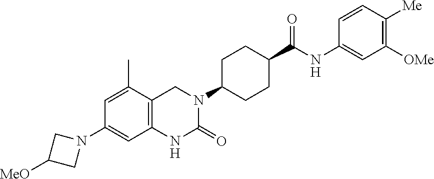 [SMILES: COc1cc(NC(=O)[C@H]2CC[C@@H](N3Cc4c(C)cc(N5CC(OC)C5)cc4NC3=O)CC2)ccc1C]

### (1s,4s)-N-(3-Methoxy-4-methylphenyl)-4-(7-(3-methoxyazetidin-1-yl)-5-methyl-2-oxo-1,2-dihydroquinazolin-3(4H)-yl)cyclohexanecarboxamide

To a microwave vial was added (1s,4s)-4-(7-bromo-5-methyl-2-oxo-1,2-dihydroquinazolin-3(4H)-yl)-N-(3-methoxy-4-methylphenyl)cyclohexanecarboxamide (50.0 mg, 103 μmol) (Example 74), XPhos Pd G3 (8.97 mg, 10.3 μmol), cesium carbonate (76.9 mg, 231 μmol), 3-methoxyazetidine hydrochloride (50.8 mg, 411 μmol) and 2-dicyclohexylphosphino-2′,4′,6′-triisopropylbiphenyl (2.53 mg, 5.14 μmol). The vial was sealed with a microwave cap purged with nitrogen (under vacuum+nitrogen backfilled, 3 times). In the meantime, 1,4-dioxane was degassed by bubbling using a nitrogen stream for 15 minutes. Degassed 1,4-Dioxane (1.00 mL) was added to the reaction flask and it was heated to 100° C. for 3 h. The reaction was then cooled to room temperature. Solvent was evaporated to dryness. Crude residue was purified by reverse phase flash chromatography on C18 (30 g, 2 to 60% MeCN in water-10 mM ammonium formate pH=3.8). Pure fractions were directly lyophilized to yield (1s,4s)-N-(3-Methoxy-4-methylphenyl)-4-(7-(3-methoxyazetidin-1-yl)-5-methyl-2-oxo-1,2-dihydroquinazolin-3(4H)-yl)cyclohexanecarboxamide (8.91 mg, 18% yield) as a pale yellow solid. LCMS ESI m/z 493.4 [M+H]⁺. ¹H NMR (400 MHz, DMSO-d₆) δ 9.66 (s, 1H), 8.83 (s, 1H), 7.26 (d, J=1.6 Hz, 1H), 7.09 (dd, J=8.1, 1.7 Hz, 1H), 7.00 (d, J=8.0 Hz, 1H), 5.81 (d, J=1.7 Hz, 1H), 5.68 (d, J=2.0 Hz, 1H), 4.28-4.22 (m, 1H), 4.22-4.12 (m, 1H), 4.09 (s, 2H), 3.92 (t, J=7.0 Hz, 2H), 3.73 (s, 3H), 3.45 (dd, J=7.9, 4.4 Hz, 2H), 3.20 (s, 3H), 2.64-2.59 (m, 1H), 2.15-2.08 (m, 2H), 2.06 (s, 3H), 2.05 (s, 3H), 2.02-1.90 (m, 2H), 1.67-1.56 (m, 2H), 1.45-1.37 (m, 2H).

### Example 30

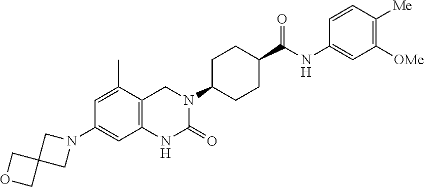 [SMILES: COc1cc(NC(=O)[C@H]2CC[C@@H](N3Cc4c(C)cc(N5CC6(COC6)C5)cc4NC3=O)CC2)ccc1C]

### (1s,4s)-N-(3-Methoxy-4-methylphenyl)-4-(5-methyl-2-oxo-7-(2-oxa-6-azaspiro[3.3]heptan-6-yl)-1,2-dihydroquinazolin-3(4H)-yl)cyclohexanecarboxamide

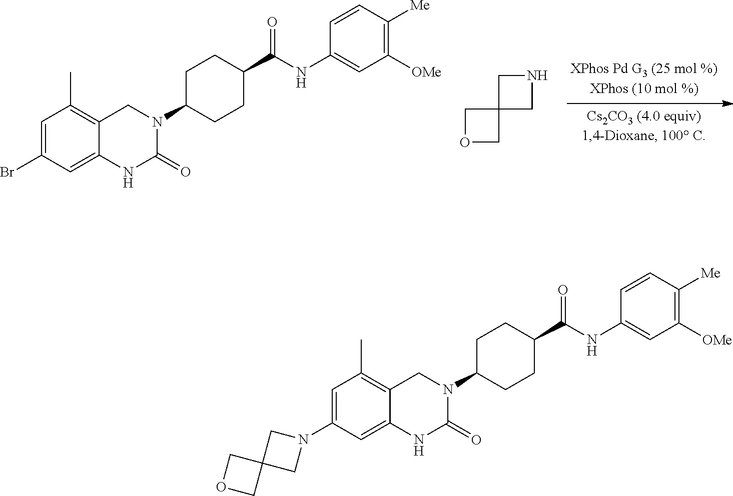 [SMILES: COc1cc(NC(=O)[C@H]2CC[C@@H](N3Cc4c(C)cc(Br)cc4NC3=O)CC2)ccc1C ; C1NCC12COC2 ; COc1cc(NC(=O)[C@H]2CC[C@@H](N3Cc4c(C)cc(N5CC6(COC6)C5)cc4NC3=O)CC2)ccc1C]

### Step 1

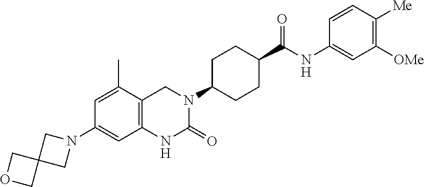 [SMILES: COc1cc(NC(=O)[C@H]2CC[C@@H](N3Cc4c(C)cc(N5CC6(COC6)C5)cc4NC3=O)CC2)ccc1C]

### (1s,4s)-N-(3-Methoxy-4-methylphenyl)-4-(5-methyl-2-oxo-7-(2-oxa-6-azaspiro[3.3]heptan-6-yl)-1,2-dihydroquinazolin-3(4H)-yl)cyclohexanecarboxamide

To a microwave vial was added (1s,4s)-4-(7-bromo-5-methyl-2-oxo-1,2-dihydroquinazolin-3(4H)-yl)-N-(3-methoxy-4-methylphenyl)cyclohexanecarboxamide (50.0 mg, 103 μmol) (Example 74), 2-Oxa-6-azaspiro[3.3]heptane (40.8 mg, 411 μmol), cesium carbonate (85.4 mg, 257 μmol), XPhos Pd G3 (22.4 mg, 25.7 μmol) and 2-dicyclohexylphosphino-2′,4′,6′-triisopropylbiphenyl (5.05 mg, 10.3 μmol). The vial was capped and purged with nitrogen under vacuum (nitrogen back filled) 3 times. In the meantime, 1,4-dioxane was degassed using nitrogen bubbling for 15 minutes. Then, degassed 1,4-Dioxane (514 μL) was added to the reaction mixture and it was heated to 100° C. for 2 h. The reaction mixture was filtered through Celite and the filtrate was concentrated under reduced pressure. The crude residue was purified by reverse phase flash chromatography on C18 (30 g column, 2 to 60% MeCN in water-10 mM ammonium formate pH=3.8). Pure fractions were directly lyophilized to yield (1s,4s)-N-(3-methoxy-4-methylphenyl)-4-(5-methyl-2-oxo-7-(2-oxa-6-azaspiro[3.3]heptan-6-yl)-1,2-dihydroquinazolin-3(4H)-yl)cyclohexanecarboxamide (29.2 mg, 56% yield) as a pale yellow solid. LCMS ESI m/z 505.4 [M+H]⁺. 1H NMR (400 MHz, DMSO-d₆) δ 9.66 (s, 1H), 8.84 (s, 1H), 7.26 (d, J=1.6 Hz, 1H), 7.09 (dd, J=8.0, 1.8 Hz, 1H), 7.00 (d, J=8.1 Hz, 1H), 5.80 (d, J=1.7 Hz, 1H), 5.68 (d, J=1.9 Hz, 1H), 4.67 (s, 4H), 4.15 (tt, J=11.9, 3.3 Hz, 1H), 4.08 (s, 2H), 3.83 (s, 4H), 3.73 (s, 3H), 2.66-2.57 (m, 1H), 2.13-2.07 (m, 2H), 2.06 (s, 3H), 2.05 (s, 3H), 1.96 (qd, J=12.5, 2.8 Hz, 2H), 1.69-1.56 (m, 2H), 1.46-1.35 (m, 2H).

### Example 31

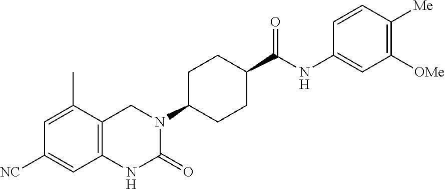 [SMILES: COc1cc(NC(=O)[C@H]2CC[C@@H](N3Cc4c(C)cc(C#N)cc4NC3=O)CC2)ccc1C]

### (1s,4s)-4-(7-Cyano-5-methyl-2-oxo-1,2-dihydroquinazolin-3(4H)-yl)-N-(3-methoxy-4-methylphenyl)cyclohexanecarboxamide

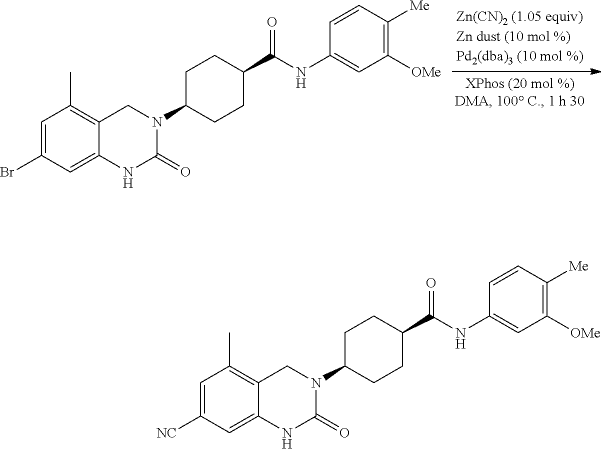 [SMILES: COc1cc(NC(=O)[C@H]2CC[C@@H](N3Cc4c(C)cc(Br)cc4NC3=O)CC2)ccc1C ; COc1cc(NC(=O)[C@H]2CC[C@@H](N3Cc4c(C)cc(C#N)cc4NC3=O)CC2)ccc1C]

### Step 1

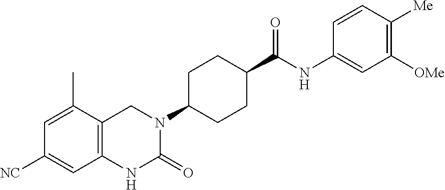 [SMILES: COc1cc(NC(=O)[C@H]2CC[C@@H](N3Cc4c(C)cc(C#N)cc4NC3=O)CC2)ccc1C]

### (1s,4s)-4-(7-Cyano-5-methyl-2-oxo-1,2-dihydroquinazolin-3(4H)-yl)-N-(3-methoxy-4-methylphenyl)cyclohexanecarboxamide

A microwave vial was loaded with 2-dicyclohexylphosphino-2′,4′,6′-triisopropylbiphenyl (10.1 mg, 20.6 μmol), tris(dibenzylideneacetone)-dipalladium (0) (9.41 mg, 10.3 μmol), Zinc (673 μg, 10.3 μmol), Zinc cyanide (12.7 mg, 108 μmol), (1s,4s)-4-(7-bromo-5-methyl-2-oxo-1,2-dihydroquinazolin-3(4H)-yl)-N-(3-methoxy-4-methylphenyl)cyclohexanecarboxamide (Example 74) (50.0 mg, 103 μmol) and was capped and purged with nitrogen using vacuum and nitrogen backfilling (3 times). In the meantime, DMA (2.06 mL) was degassed using a stream of nitrogen bubbled in the solvent for 15 minutes. Degassed DMA (2.06 mL) was added to the reagents and the reaction mixture was heated and stirred at 100° C. for 1 h. The reaction mixture was cooled to room temperature and filtered through Celite. The filtrate was directly loaded on C18 column and the product was eluted from 20 to 70% MeCN in water-10 mM ammonium formate pH=3.8. Pure fractions were directly lyophilized to yield (1s,4s)-4-(7-cyano-5-methyl-2-oxo-1,2-dihydroquinazolin-3(4H)-yl)-N-(3-methoxy-4-methylphenyl)cyclohexanecarboxamide (17.7 mg, 40% yield) as a white solid. LCMS ESI m/z 433.3 [M+H]⁺. ¹H NMR (400 MHz, DMSO-d₆) δ 9.67 (s, 1H), 9.38 (s, 1H), 7.27 (d, J=1.6 Hz, 1H), 7.16 (s, 1H), 7.09 (dd, J=8.0, 1.8 Hz, 1H), 7.00 (d, J=8.4 Hz, 1H), 6.88 (s, 1H), 4.28 (s, 2H), 4.22-4.12 (m, 1H), 3.73 (s, 3H), 2.67-2.58 (m, 1H), 2.19 (s, 3H), 2.10 (d, J=13.4 Hz, 2H), 2.06 (s, 3H), 1.99 (qd, J=12.7, 3.2 Hz, 2H), 1.69-1.57 (m, 2H), 1.49-1.39 (m, 2H).

### Example 32

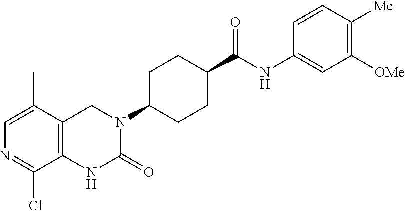 [SMILES: COc1cc(NC(=O)[C@H]2CC[C@@H](N3Cc4c(C)cnc(Cl)c4NC3=O)CC2)ccc1C]

### (1s,4s)-4-(8-Chloro-5-methyl-2-oxo-1,2-dihydropyrido[3,4-d]pyrimidin-3(4H)-yl)-N-(3-methoxy-4-methylphenyl)cyclohexanecarboxamide

(1s,4s)-4-(8-Chloro-5-methyl-2-oxo-1,2-dihydropyrido[3,4-d]pyrimidin-3(4H)-yl)-N-(3-methoxy-4-methylphenyl)cyclohexanecarboxamide was made from procedures and steps depicted in Example 20. The product was isolated (198 mg, 93% yield) as a white solid. LCMS ESI m/z 443.0 [M+H]⁺. ¹H NMR (400 MHz, DMSO-d₆) δ 9.67 (s, 1H), 8.60 (s, 1H), 7.76 (s, 1H), 7.28 (d, J=1.6 Hz, 1H), 7.09 (dd, J=8.0, 1.7 Hz, 1H), 7.00 (d, J=8.3 Hz, 1H), 4.31 (s, 2H), 4.19 (tt, J=11.9, 3.6 Hz, 1H), 3.73 (s, 3H), 2.68-2.59 (m, 1H), 2.14 (s, 3H), 2.13-2.08 (m, 2H), 2.06 (s, 3H), 1.99 (qd, J=12.5, 3.3 Hz, 2H), 1.73-1.60 (m, 2H), 1.51-1.41 (m, 2H).

### Example 33

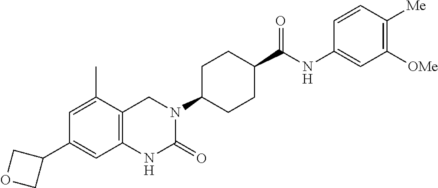 [SMILES: COc1cc(NC(=O)[C@H]2CC[C@@H](N3Cc4c(C)cc(C5COC5)cc4NC3=O)CC2)ccc1C]

### (1s,4s)-N-(3-Methoxy-4-methylphenyl)-4-(5-methyl-7-(oxetan-3-yl)-2-oxo-1,2-dihydroquinazolin-3(4H)-yl)cyclohexanecarboxamide

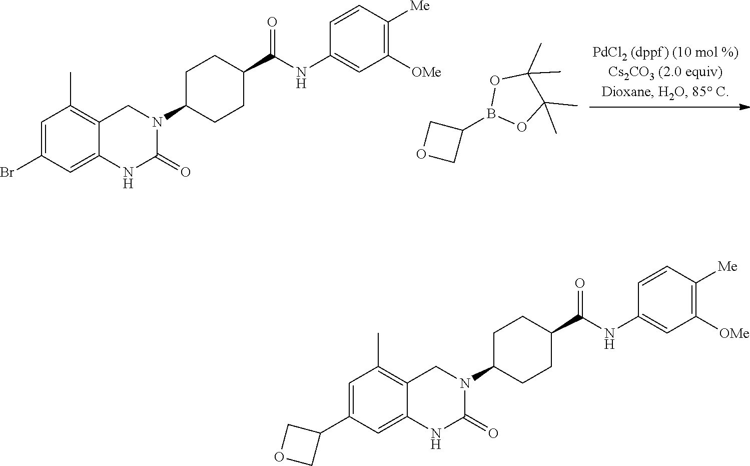 [SMILES: COc1cc(NC(=O)[C@H]2CC[C@@H](N3Cc4c(C)cc(Br)cc4NC3=O)CC2)ccc1C ; CC1(C)OB(C2COC2)OC1(C)C ; COc1cc(NC(=O)[C@H]2CC[C@@H](N3Cc4c(C)cc(C5COC5)cc4NC3=O)CC2)ccc1C]

### Step 1

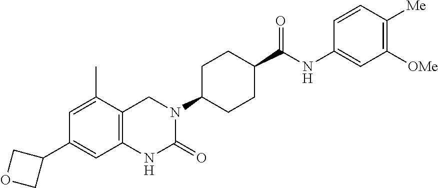 [SMILES: COc1cc(NC(=O)[C@H]2CC[C@@H](N3Cc4c(C)cc(C5COC5)cc4NC3=O)CC2)ccc1C]

### (1s,4s)-N-(3-Methoxy-4-methylphenyl)-4-(5-methyl-7-(oxetan-3-yl)-2-oxo-1,2-dihydroquinazolin-3(4H)-yl)cyclohexanecarboxamide

A microwave vial was charged with 4,4,5,5-tetramethyl-2-(oxetan-3-yl)-1,3,2-dioxaborolane (21.9 mg, 113 μmol), cesium carbonate (67.0 mg, 206 μmol), (1s,4s)-4-(7-bromo-5-methyl-2-oxo-1,2-dihydroquinazolin-3(4H)-yl)-N-(3-methoxy-4-methylphenyl)cyclohexanecarboxamide (Example 74) (50.0 mg, 103 μmol) and 1,4-dioxane (467 μL) and Water (93.5 μL). The vial was capped with a septum and purged with nitrogen for 15 minutes. 1,1′-Bis(diphenylphosphino)ferrocene dichloropalladium (II) (7.68 mg, 10.3 μmol) was added, the vial was purged for another minute and capped under nitrogen before heating to 85° C. for 16 h. Reaction was cooled to room temperature and silica gel was added. Purification by flash chromatography was done (dry loading, 25 g column, from 1 to 60% (20% MeOH in DCM) in DCM) to yield 10 mg of product. This material was purified again by reverse phase flash chromatography on C18 (30 g column, 2 to 60% MeCN in water-10 mM Ammonium bicarbonate pH=10). Pure fractions were directly lyophilized to yield (1s,4s)-N-(3-methoxy-4-methylphenyl)-4-(5-methyl-7-(oxetan-3-yl)-2-oxo-1,2-dihydroquinazolin-3(4H)-yl)cyclohexanecarboxamide (3.27 mg, 6.9% yield) as a white solid. LCMS ESI m/z 464.3 [M+H]⁺. ¹H NMR (400 MHz, DMSO-d₆) δ 9.67 (s, 1H), 9.03 (s, 1H), 7.27 (s, 1H), 7.10 (dd, J=8.1, 1.8 Hz, 1H), 7.00 (d, J=8.2 Hz, 1H), 6.75 (s, 1H), 6.60 (s, 1H), 6.36 (d, J=15.9 Hz, 1H), 6.18 (dt, J=15.9, 5.1 Hz, 1H), 4.80 (t, J=5.6 Hz, 1H), 4.26-4.12 (m, 2H), 4.19 (s, 2H), 4.06 (t, J=4.6 Hz, 1H), 3.74 (s, 3H), 2.64-2.60 (m, 1H), 2.13 (s, 3H), 2.12-2.08 (m, 2H), 2.06 (s, 3H), 2.02-1.91 (m, 2H), 1.70-1.58 (m, 2H), 1.48-1.39 (m, 2H).

### Example 34

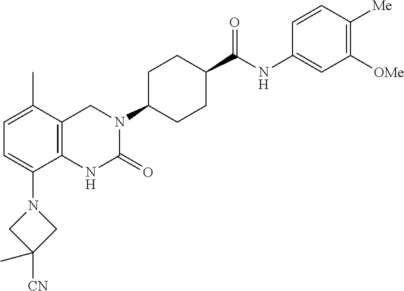 [SMILES: COc1cc(NC(=O)[C@H]2CC[C@@H](N3Cc4c(C)ccc(N5CC(C)(C#N)C5)c4NC3=O)CC2)ccc1C]

### (1s,4s)-4-(8-(3-Cyano-3-methylazetidin-1-yl)-5-methyl-2-oxo-1,2-dihydroquinazolin-3(4H)-yl)-N-(3-methoxy-4-methylphenyl)cyclohexanecarboxamide

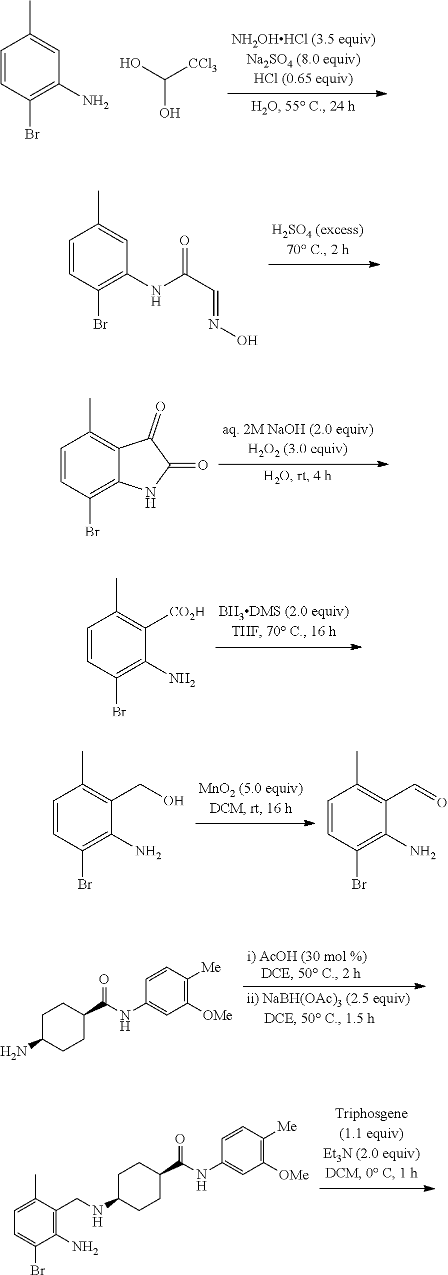

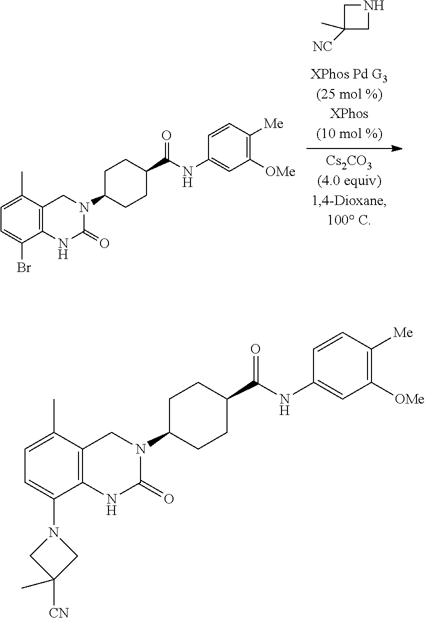

### Step 1

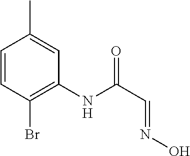

### (E)-N-(2-Bromo-5-methylphenyl)-2-(hydroxyimino)acetamide

Sodium sulfate (122 g, 860 mmol) was dissolved in water (726 mL) in a 2000 mL Erlenmeyer. Then, hydroxylamine hydrochloride (27.4 g, 387 mmol), 2-bromo-5-methylaniline (14.4 mL, 107 mmol) and an 2 M aqueous solution of concentrated hydrochloric acid (36.0 mL, 72.0 mmol) were added (a white precipitate of the aniline sulfate was formed). The mixture was heated at 55° C. and chloral hydrate (21.3 g, 129 mmol) was added. The suspension was stirred with strong agitation at 55° C. overnight. After that period, a gum precipitated out of the solution and was filtrated through a Buchner funnel and washed thoroughly with water (500 mL) and then heptanes (4×400 mL). The solid was then dried over high vacuum overnight providing crude (85% pure) (E)-N-(2-Bromo-5-methylphenyl)-2-(hydroxyimino)acetamide (27.6 g, 100% yield) which was used as is for the next step. ¹H NMR (400 MHz, DMSO-d₆) δ12.42 (s, 1H), 9.39 (s, 1H), 7.72 (d, J=1.1 Hz, 1H), 7.63 (s, 1H), 7.53 (d, J=8.2 Hz, 1H), 6.96 (dd, J=8.3, 1.8 Hz, 1H), 2.27 (s, 3H).

### Step 2

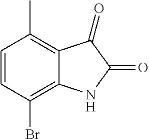

### 7-Bromo-4-methylindoline-2,3-dione

Crude (E)-N-(2-bromo-5-methylphenyl)-2-(hydroxyimino)acetamide (28.0 g, 109 mmol) was added portionwise to a rapidly stirring solution of concentrated sulfuric acid (80 mL, 109 mmol) warmed to 55° C. (at a rate to keep the reaction temperature between 55-70° C.). The resulting solution was warmed to 70° C. and stirred for 20 min and then cooled to ambient temperature. The purplish-red mixture was poured carefully onto crushed ice (ca. 1000 g) and the mixture was allowed to stand for 1 h. The purple precipitate was collected by filtration, washed with water (3×200 mL), and then dried under vacuum and gave crude 7-bromo-4-methylindoline-2,3-dione (26.1 g, 100% yield) as a brown solid. Used as is for the next reaction. ¹H NMR (400 MHz, DMSO-d₆) δ 11.23 (s, 1H), 7.62 (d, J=8.3 Hz, 1H), 6.82 (d, J=8.4 Hz, 1H), 2.40 (s, 3H).

### Step 3

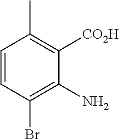

### 2-Amino-3-bromo-6-methylbenzoic Acid

To an ice-cold solution of 7-bromo-4-methylindoline-2,3-dione (27.0 g, 112 mmol) in 2 M sodium hydroxide (112 mL, 225 mmol) in water was added hydrogen peroxide (35 mL, 337 mmol) dropwise. The mixture was warmed to room temperature and stirred for 2 hours. The mixture was carefully acidified by addition of a saturated citric acid solution. The solid precipitate was collected by filtration, washed with water, and dried under vacuum. A dark brown solid was isolated. 2-Amino-3-bromo-6-methylbenzoic acid was isolated and used as is for the next step (10.5 g, 41% yield over 3 steps). ¹H NMR (400 MHz, DMSO-d₆) δ 7.37 (d, J=8.1 Hz, 1H), 6.40 (d, J=8.1 Hz, 1H), 2.28 (s, 3H).

### Step 4

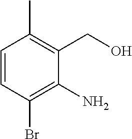

### (2-Amino-3-bromo-6-methylphenyl)methanol

To a flame-dried, nitrogen-flushed 100 mL round-bottom flask was added 2-amino-3-bromo-6-methylbenzoic acid (1.85 g, 8.06 mmol). It was dissolved in anhydrous THF (40.3 mL), and it was then cooled to 0° C. using a water ice bath. Borane dimethylsulfide complex (8.06 mL, 16.1 mmol) in THF was then added dropwise generating gas during the addition. After gas evolution subsided, the reaction was heated to reflux (70° C.) in a oil bath for 16 hours. After that period, the reaction was cooled to room temperature and quenched carefully by addition of MeOH (10 mL). The reaction was stirred at room temperature for 1 hour and then evaporated to dryness. (2-Amino-3-bromo-6-methylphenyl) methanol (1.74 g, 100% yield) isolated as a yellow solid. The product was carried over the next step. ¹H NMR (400 MHz, CDCl₃) δ 7.30 (d, J=8.1 Hz, 1H), 6.60 (d, J=8.4 Hz, 1H), 4.83 (s, 2H), 3.47 (d, J=11.7 Hz, 1H), 2.29 (s, 3H).

### Step 5

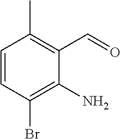

### 2-Amino-3-bromo-6-methylbenzaldehyde

To a 100 mL round-bottom flask was added (2-amino-3-bromo-6-methylphenyl) methanol (1.63 g, 7.56 mmol) and it was diluted with DCM (37.8 mL). The reaction was initiated by addition of manganese (IV) oxide activated (3.65 g, 37.8 mmol). The reaction was stirred at room temperature until judged to be finished. After 4 hours, the suspension was then filtered through Celite and the filtrate was evaporated to dryness. It resulted in a clear yellow oil which solidified over time to a greenish solid. 2-Amino-3-bromo-6-methylbenzaldehyde (1.40 g, 85% yield) was carried over the next step without purification 1H NMR (400 MHz, CDCl₃) δ 10.30 (s, 1H), 7.47 (d, J=7.9 Hz, 1H), 6.37 (d, J=7.9 Hz, 1H), 2.55 (s, 3H).

### Step 6

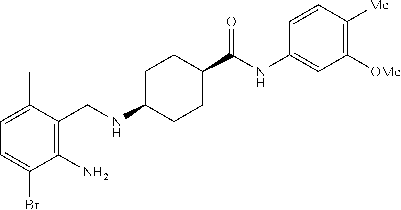 [SMILES: COc1cc(NC(=O)[C@H]2CC[C@@H](NCc3c(C)ccc(Br)c3N)CC2)ccc1C]

### (1s,4s)-4-((2-Amino-3-bromo-6-methylbenzyl)amino)-N-(3-methoxy-4-methylphenyl)cyclohexanecarboxamide

To a 100 mL round-bottom flask which contained 2-amino-3-bromo-6-methylbenzaldehyde (1.40 g, 6.54 mmol) was added anhydrous DCE (32.7 mL). Then, (1s,4s)-4-amino-N-(3-methoxy-4-methylphenyl)cyclohexanecarboxamide (2.57 g, 9.81 mmol) and AcOH (112 μL, 1.96 mmol) was added. The solution was stirred for 2 hours at 50° C. Then, sodium triacetoxyborohydride (3.56 g, 16.4 mmol) were added in one portion and the reaction was then heated to 50° C. to promote condensation/reduction. Reaction was cooled to room temperature and the crude was evaporated to dryness which resulted in a gummy black solid. The gum was purified by C18 reverse chromatography using a gradient of 100% AMF to 60% MeCN in 40% AMF. The fractions containing the desired product were combined and evaporated to dryness. The product was carried to the next step as is. (1s,4s)-4-((2-Amino-3-bromo-6-methylbenzyl)amino)-N-(3-methoxy-4-methylphenyl)cyclohexanecarboxamide (1.17 g, 39% yield) obtained as a white solid. LCMS ESI m/z 458.4, 460.4 [M+H]⁺.

### Step 7

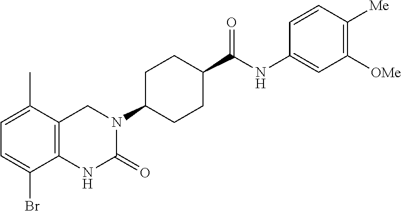 [SMILES: COc1cc(NC(=O)[C@H]2CC[C@@H](N3Cc4c(C)ccc(Br)c4NC3=O)CC2)ccc1C]

### (1s,4s)-4-(8-Bromo-5-methyl-2-oxo-1,2-dihydroquinazolin-3(4H)-yl)-N-(3-methoxy-4-methylphenyl)cyclohexanecarboxamide

To a 250 mL round-bottom flask was added (1s,4s)-4-((2-Amino-3-bromo-6-methylbenzyl)amino)-N-(3-methoxy-4-methylphenyl)cyclohexanecarboxamide (140 mg, 304 μmol). It was diluted in DCM (12.7 mL). The reaction was initiated by addition of triphosgene (846 mg, 2.80 mmol) and triethylamine (712 μL, 5.08 mmol) dropwise. The reaction was found to be slightly exothermic. The reaction was found to be done after 3 hours. The reaction was stopped by evaporation of the crude to dryness. It resulted in a white solid. Purification using a gradient of 40% EtOAc in heptanes to 100% EtOAc provided (1s,4s)-4-(8-Bromo-5-methyl-2-oxo-1,2-dihydroquinazolin-3(4H)-yl)-N-(3-methoxy-4-methylphenyl)cyclohexanecarboxamide (960 mg, 78% yield) as a white solid. LCMS ESI m/z 486.0, 488.0 [M+H]⁺. (see Example 62)

### Step 8

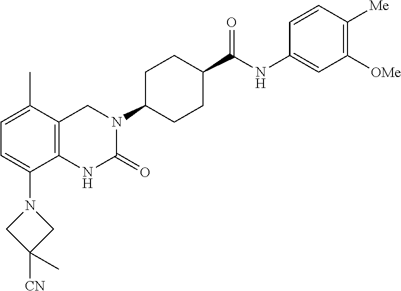 [SMILES: COc1cc(NC(=O)[C@H]2CC[C@@H](N3Cc4c(C)ccc(N5CC(C)(C#N)C5)c4NC3=O)CC2)ccc1C]

### (1s,4s)-4-(8-(3-Cyano-3-methylazetidin-1-yl)-5-methyl-2-oxo-1,2-dihydroquinazolin-3(4H)-yl)-N-(3-methoxy-4-methylphenyl)cyclohexanecarboxamide

To a flame-dried, nitrogen-flushed 0.5-2.0 mL Biotage vial was added (1s,4s)-4-(8-Bromo-5-methyl-2-oxo-1,2-dihydroquinazolin-3(4H)-yl)-N-(3-methoxy-4-methylphenyl)cyclohexanecarboxamide (25.0 mg, 51.4 μmol) and it was diluted with 1,4-dioxane (0.257 mL). The solvent was sparged with nitrogen for 5 minutes. Then, XPhos Pd G3 (8.97 mg, 10.3 μmol), cesium carbonate (76.9 mg, 231 μmol), 3-methylazetidine-3-carbonitrile hydrochloride (35.1 mg, 257 μmol) and 2-dicyclohexylphosphino-2′,4′,6′-triisopropylbiphenyl (2.53 mg, 5.14 μmol) were added in that order. The vial was sealed with a microwave cap and heated to 100° C. for 1.5 h. The reaction was then cooled to room temperature and opened to air. The solvent was evaporated to dryness. Purification over silica gel of the crude was performed using a gradient of 40% EtOAc in 60% heptanes to 100% EtOAc to 20% MeOH in 80% EtOAc. Evaporation of clean fraction to dryness provided (1s,4s)-4-(8-(3-cyano-3-methylazetidin-1-yl)-5-methyl-2-oxo-1,2-dihydroquinazolin-3(4H)-yl)-N-(3-methoxy-4-methylphenyl)cyclohexanecarboxamide as a yellow solid (7.90 mg, 29% yield). LCMS ESI m/z 502.2 [M+H]⁺. ¹H NMR (400 MHz, DMSO-d₆) δ 9.67 (s, 1H), 7.68 (s, 1H), 7.27 (s, 1H), 7.09 (d, J=7.9 Hz, 1H), 7.00 (d, J=8.1 Hz, 1H), 6.67 (d, J=8.2 Hz, 1H), 6.55 (d, J=8.3 Hz, 1H), 4.22-4.11 (m, 1H), 4.17 (s, 2H), 4.00 (d, J=7.0 Hz, 2H), 3.72 (d, J=8.3 Hz, 2H), 3.73 (s, 3H), 2.67-2.60 (m, 1H), 2.14-2.03 (m, 2H), 2.09 (s, 3H), 2.06 (s, 3H), 2.04-1.97 (m, 2H), 1.73-1.59 (m, 2H), 1.66 (s, 3H), 1.52-1.38 (m, 2H).

### Example 35

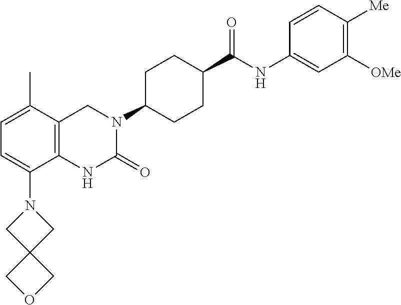 [SMILES: COc1cc(NC(=O)[C@H]2CC[C@@H](N3Cc4c(C)ccc(N5CC6(COC6)C5)c4NC3=O)CC2)ccc1C]

### (1s,4s)-N-(3-Methoxy-4-methylphenyl)-4-(5-methyl-2-oxo-8-(2-oxa-6-azaspiro[3.3]heptan-6-yl)-1,2-dihydroquinazolin-3(4H)-yl)cyclohexanecarboxamide

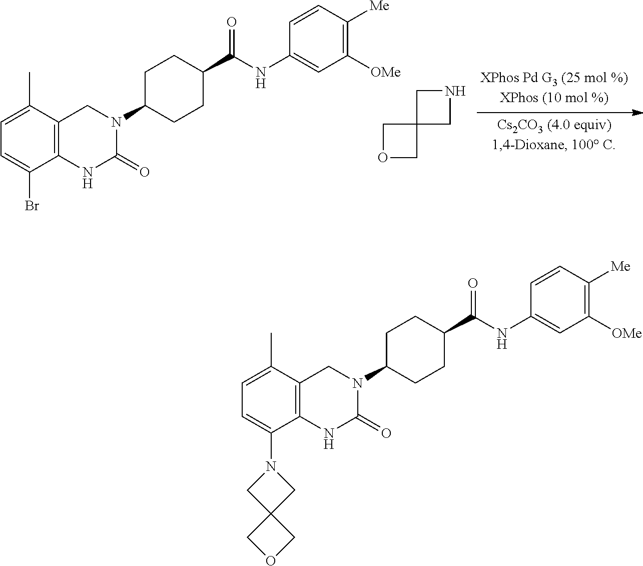

### Step 1

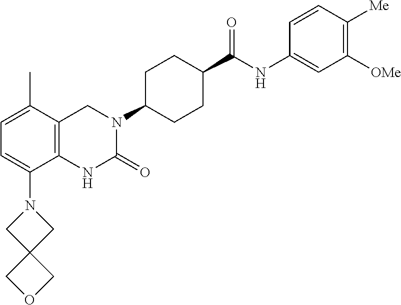 [SMILES: COc1cc(NC(=O)[C@H]2CC[C@@H](N3Cc4c(C)ccc(N5CC6(COC6)C5)c4NC3=O)CC2)ccc1C]

### (1s,4s)-N-(3-Methoxy-4-methylphenyl)-4-(5-methyl-2-oxo-8-(2-oxa-6-azaspiro[3.3]heptan-6-yl)-1,2-dihydroquinazolin-3(4H)-yl)cyclohexanecarboxamide

To a flame-dried, nitrogen-flushed 0.5-2.0 mL Biotage vial was added (1s,4s)-4-(8-Bromo-5-methyl-2-oxo-1,2-dihydroquinazolin-3(4H)-yl)-N-(3-methoxy-4-methylphenyl)cyclohexanecarboxamide from Example 62 (25.0 mg, 51.4 μmol) and it was diluted with 1,4-Dioxane (257 mL). The solvent was sparged with nitrogen for 5 minutes. Then, XPhos Pd G3 (8.97 mg, 10.3 μmol), cesium carbonate (76.9 mg, 231 μmol), 2-Oxa-6-azaspiro[3.3]heptane (20.4 mg, 206 μmol) and 2-dicyclohexylphosphino-2′,4′,6′-triisopropylbiphenyl (2.53 mg, 5.14 μmol) were added in that order. The vial was sealed with a microwave cap and heated to 100° C. overnight. The reaction was then cooled to room temperature and opened to air. The solvent was evaporated to dryness. Puh to 100% EtOAc to 20% MeOH in 80% EtOAc. Evaporation of clean fractions to dryness provided (1s,4s)-N-(3-Methoxy-4-methylphenyl)-4-(5-methyl-2-oxo-8-(2-oxa-6-azaspiro[3.3]heptan-6-yl)-1,2-dihydroquinazolin-3(4H)-yl)cyclohexanecarboxamide which was lyophilized to provide as a yellow solid (10.5 mg, 37% yield). LCMS ESI m/z 505.3 [M+H]⁺. ¹H NMR (400 MHz, DMSO-d₆) δ 9.67 (s, 1H), 7.35 (s, 1H), 7.27 (s, 1H), 7.08 (s, 1H), 7.00 (d, J=8.1 Hz, 1H), 6.63 (d, J=8.4 Hz, 1H), 6.46 (d, J=7.7 Hz, 1H), 4.67 (s, 4H), 4.20-4.11 (m, 1H), 4.16 (s, 2H), 3.86 (s, 4H), 3.73 (s, 3H), 2.67-2.61 (m, 1H), 2.14-2.06 (m, 2H), 2.07 (s, 3H), 2.06 (s, 3H) 2.01-1.92 (m, 2H), 1.71-1.58 (m, 2H), 1.48-1.39 (m, 2H).

### Example 36

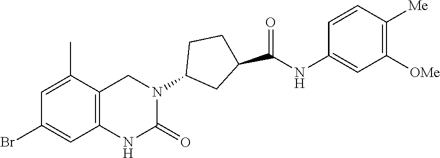 [SMILES: COc1cc(NC(=O)[C@@H]2CC[C@@H](N3Cc4c(C)cc(Br)cc4NC3=O)C2)ccc1C]

### (1R,3R)-3-(7-Bromo-5-methyl-2-oxo-1,2-dihydroquinazolin-3(4H)-yl)-N-(3-methoxy-4-methylphenyl)cyclopentanecarboxamide

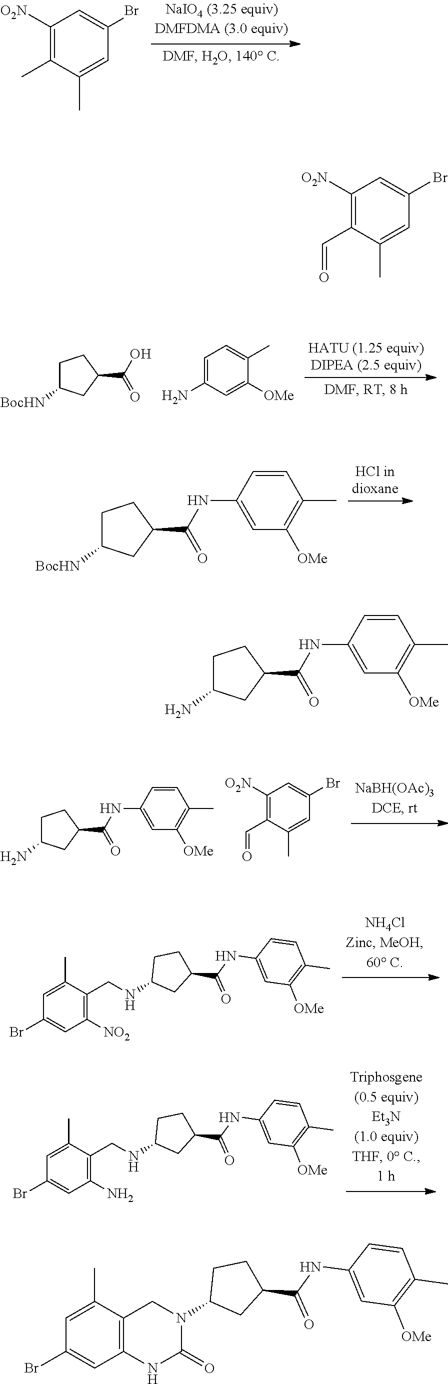

### Step 1

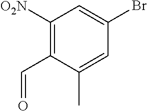

### 4-Bromo-2-methyl-6-nitrobenzaldehyde

To a stirred solution of 5-bromo-1,2-dimethyl-3-nitrobenzene (1.00 g, 4.26 mmol) in DMF (10.6 mL) was added N,N-dimethylformamide dimethyl acetal (DMFDMA) (1.70 mL, 12.8 mmol) After being heated at 140° C. for 24 h, the dark red solution was cooled to room temperature and added quickly to a rapidly stirred solution of sodium periodate (NaIO₄) (2.99 g, 13.8 mmol) in water (7.5 mL) and DMF (2.5 mL) at 0° C. The reaction flask was washed with DMF (2 mL) at 0° C. and added to NaIO₄ mixture. The reaction was stirred at 0° C. for 3 h before being warmed to rt. The orange solution was filtered and rinsed with EtOAc (80 mL). The filtrate was then washed with H₂O (3×25 mL) and sat brine (3×25 mL). The dried (Na₂SO₄) extract was concentrated in vacuo to a dark orange oil and purified via flash chromatography over silica gel eluting from 10-70% EtOAc/heptanes to yield 4-bromo-2-methyl-6-nitrobenzaldehyde (228 mg, 22% yield) pale orange solid. LCMS ESI m/z 244.0 [M+H]⁺. ¹H NMR (400 MHz, CDCl₃) δ 10.28 (s, 1H), 8.11 (d, J=1.5 Hz, 1H), 7.74 (s, 1H), 2.50 (s, 3H).

### Step 2

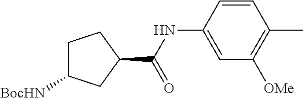 [SMILES: COc1cc(NC(=O)[C@@H]2CC[C@@H](NC(=O)OC(C)(C)C)C2)ccc1C]

### tert-Butyl ((1R,3R)-3-((3-methoxy-4-methylphenyl)carbamoyl)cyclopentyl)carbamate

To a solution of (1R,3R)-3-((tert-butoxycarbonyl)amino)cyclopentanecarboxylic acid (500 mg, 2.14 mmol) in DMF (10.7 mL) was added HATU (995 mg, 2.56 mmol) and N,N-diisopropylethylamine (931 μL, 5.34 mmol) and was left to stir for 30 minutes. Then, 3-Methoxy-4-methylaniline (299 mg, 2.14 mmol) was added and the reaction left to stir for 8 hours. After full conversion, brine and EtOAc 1:1 were added and the mixture was extracted x3 with EtOAc. The combined organic layers were washed with brine and concentrated under vacuum to afford tert-Butyl ((1R,3R)-3-((3-methoxy-4-methylphenyl)carbamoyl)cyclopentyl) carbamate (745 mg, 100% yield) as a light white powder. LCMS ESI m/z 293.3 [M-tBu]⁺.

### Step 2

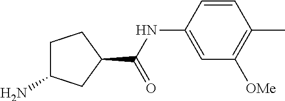 [SMILES: COc1cc(NC(=O)[C@@H]2CC[C@@H](N)C2)ccc1C]

### (1R,3R)-3-Amino-N-(3-methoxy-4-methylphenyl)cyclopentanecarboxamide

To a solution of tert-Butyl ((1R,3R)-3-((3-methoxy-4-methylphenyl)carbamoyl)cyclopentyl) carbamate (745 mg, 2.14 mmol) in DCM (4.28 mL) was added hydrochloric acid (3.74 mL, 15.0 mmol). The solution was left to stir until disappearance of starting material was observed. The crude reaction mixture was then concentrated under vacuum and the resulting residue was dissolved in DMSO and subjected to reverse phase chromatography (C18, 100% AMF to 100% MeCN). The fractions containing product were concentrating under vacuum to afford (1R,3R)-3-amino-N-(3-methoxy-4-methylphenyl)cyclopentanecarboxamide (160 mg, 30% yield) as a white solid. LCMS ESI m/z 249.3 [M+H]⁺.

### Step 3

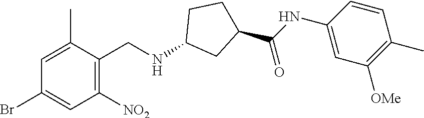 [SMILES: COc1cc(NC(=O)[C@@H]2CC[C@@H](NCc3c(C)cc(Br)cc3[N+](=O)[O-])C2)ccc1C]

### (1R,3R)-3-((4-bromo-2-methyl-6-nitrobenzyl)amino)-N-(3-methoxy-4-methylphenyl)cyclopentanecarboxamide

To a solution of (1R,3R)-3-amino-N-(3-methoxy-4-methylphenyl)cyclopentanecarboxamide (155 mg, 527 μmol) in DCE (2.93 mL) was added-bromo-2-methyl-6-nitrobenzaldehyde (143 mg, 585 μmol). After 2 hours of stirring, sodium triacetoxyborohydride (127 mg, 585 μmol) was added quickly. The solution was left to stir until disappearance of amine was observed. The reaction was quenched with water and diluted with EtOAc. The solution was extracted x3 with EtOAc. The organic layers were pooled and concentrated under vacuum to afford a yellow residue. The residue was dissolved in DMSO and subjected to reverse phase chromotography using a gradient of Formic acid buffered water: MeCN (5-95%). The fractions collected were concentrated under vacumm to afford (1R,3R)-3-((4-bromo-2-methyl-6-nitrobenzyl)amino)-N-(3-methoxy-4-methylphenyl)cyclopentanecarboxamide (140 mg, 50% yield) as a light yellow powder. LCMS ESI m/z 478.3 [M+H]⁺.

### Step 4

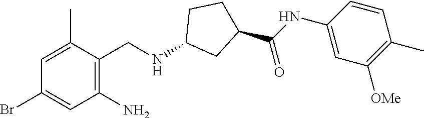 [SMILES: COc1cc(NC(=O)[C@@H]2CC[C@@H](NCc3c(C)cc(Br)cc3N)C2)ccc1C]

### (1R,3R)-3-((2-Amino-4-bromo-6-methylbenzyl)amino)-N-(3-methoxy-4-methylphenyl)cyclopentanecarboxamide

To a solution of (1R,3R)-3-((4-bromo-2-methyl-6-nitrobenzyl)amino)-N-(3-methoxy-4-methylphenyl)cyclopentanecarboxamide (30.0 mg, 63.0 μmol) in MeOH (500 μL) was added ammonium chloride (13.5 mg, 252 μmol) and Zinc (2.36 μL, 252 μmol). The solution was warmed to 60° C. and stirred for 2 hours. The solution was then filtered through a 0.45 μm syringe filter. The resulting solution was suspended in EtOAc and saturated sodium bicarbonate. The solution was extracted ×3 with EtOAc. The pooled organic layers were concentrated under vacuum to afford (1R,3R)-3-((2-Amino-4-bromo-6-methylbenzyl)amino)-N-(3-methoxy-4-methylphenyl)cyclopentanecarboxamide (28.1 mg, 100% yield) as a white solid. The product was carried through to the next step without purification. LCMS ESI m/z 446.3 [M+H]⁺.

### Step 5

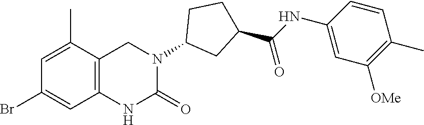 [SMILES: COc1cc(NC(=O)[C@@H]2CC[C@@H](N3Cc4c(C)cc(Br)cc4NC3=O)C2)ccc1C]

### (1R,3R)-3-(7-bromo-5-methyl-2-oxo-1,2-dihydroquinazolin-3(4H)-yl)-N-(3-methoxy-4-methylphenyl)cyclopentanecarboxamide

(1R,3R)-3-((2-Amino-4-bromo-6-methylbenzyl)amino)-N-(3-methoxy-4-methylphenyl)cyclopentanecarboxamide (28.1 mg, 63.0 μmol) was dissolved in THF (420 μL) and the resulting mixture was then cooled down to 0° C. Then, triethylamine (8.83 μL, 63.0 μmol) was added to the reaction mixture followed by triphosgene (9.35 mg, 31.5 μmol). Upon disappearance of starting material, the solution was quenched with water and extracted x3 with EtOAc. The pooled fractions were concentrated under vacuum to afford a yellow solid. The solid was dissolved in minimal DMSO and subjected to reverse-phase chromotography (AmF:MeCN, 30-95%). The fractions containing product were collected and lyophilized to afford (1R,3R)-3-(7-bromo-5-methyl-2-oxo-1,2-dihydroquinazolin-3(4H)-yl)-N-(3-methoxy-4-methylphenyl)cyclopentanecarboxamide (1.49 mg, 5% yield) as a slightly yellow solid. LCMS ESI m/z 474.1. [M+H]⁺. ¹H NMR (400 MHz, DMSO-d₆) δ 9.83 (s, 1H), 9.25 (s, 1H), 7.36 (s, 1H), 7.07-6.99 (m, 2H), 6.93 (d, J=1.2 Hz, 1H), 6.78 (d, J=1.6 Hz, 1H), 4.80 (dd, J=16.8, 8.4 Hz, 1H), 4.26 (s, 2H), 3.75 (s, 3H), 3.06-2.96 (m, 1H), 2.21 (s, 3H), 2.08 (s, 3H), 2.05-2.00 (m, 1H), 1.99-1.92 (m, 2H), 1.86-1.79 (m, 2H), 1.73 (m, 1H).

### Example 37

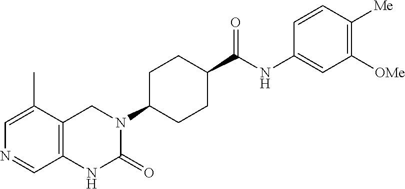 [SMILES: COc1cc(NC(=O)[C@H]2CC[C@@H](N3Cc4c(C)cncc4NC3=O)CC2)ccc1C]

### (1s,4s)-N-(3-Methoxy-4-methylphenyl)-4-(5-methyl-2-oxo-1,2-dihydropyrido[3,4-d]pyrimidin-3(4H)-yl)cyclohexanecarboxamide

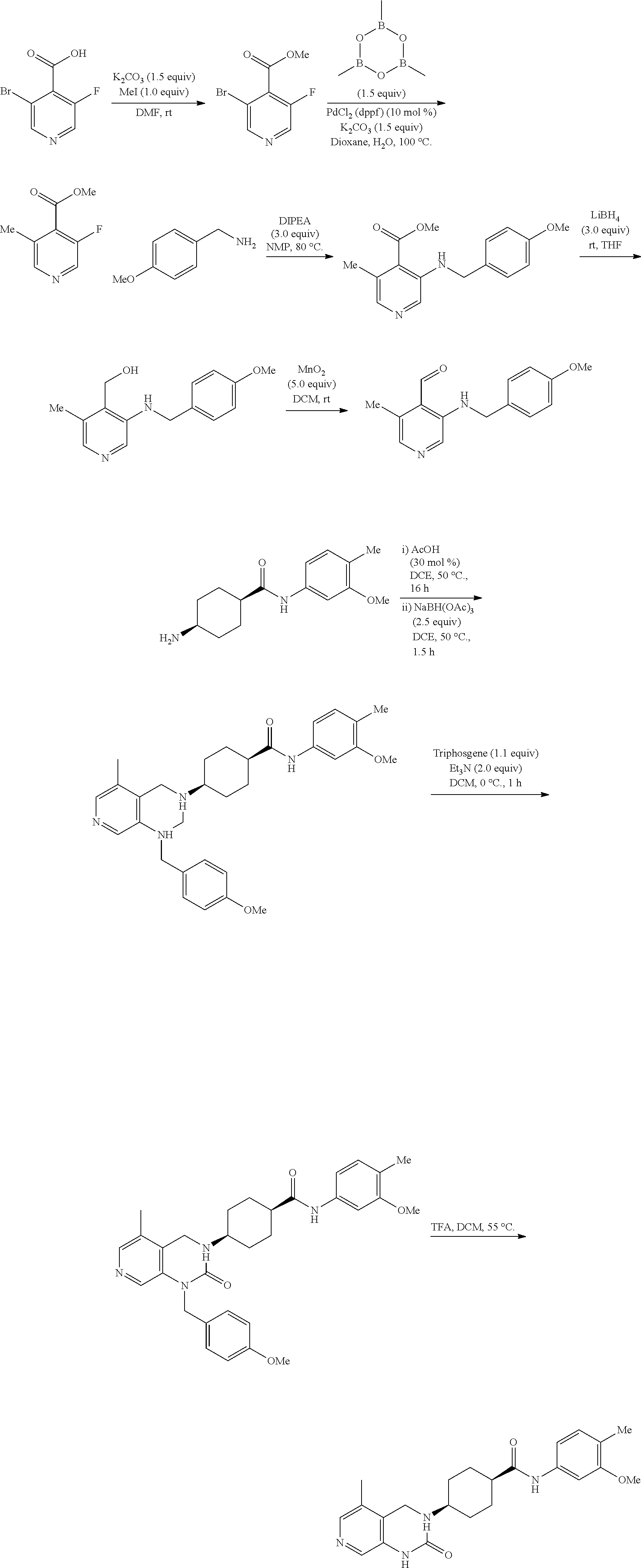

### Step 1

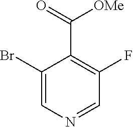 [SMILES: COC(=O)c1c(F)cncc1Br]

### Methyl 3-bromo-5-fluoroisonicotinate

To a solution of iodomethane (597 μL, 9.50 mmol) in DMF (23.8 mL) was added 3-bromo-5-fluoroisonicotinic acid (2.20 g, 9.50 mmol) and potassium carbonate (1.97 g, 14.3 mmol). The reaction mixture was stirred at room temperature for 16 h. Water was added and the product was extracted 3 times with EtOAc. Organics were combined, washed with water, brine, dried with Na₂SO₄, filtered and concentrated. The crude residue was purified by flash chromatography on silica gel (70 g column, 5 to 60% EtOAc in heptanes) to yield methyl 3-bromo-5-fluoroisonicotinate (1.88 g, 85% yield) as a translucent oil. LCMS ESI m/z 274.9 [M+H]⁺.

### Step 2

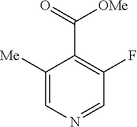 [SMILES: COC(=O)c1c(C)cncc1F]

### Methyl 3-fluoro-5-methylisonicotinate

A microwave vial was charged with potassium carbonate (472 mg, 3.42 mmol), methyl 3-fluoro-5-bromoisonicotinate (400 mg, 1.71 mmol) and trimethylboroxine (362 uL, 2.56 mmol). 1,4-dioxane (14.2 mL) and Water (2.85 mL) were added and the mixture was degassed for a 15 minutes period using a flow of nitrogen. Finally, 1,1′-Bis(diphenylphosphino)ferrocene dichloropalladium (II) (128 mg, 171 μmol) was added under nitrogen, the vial was capped and the reaction mixture was stirred at 100° C. for 16 h. The reaction mixture was cooled to room temperature. Silica gel was added directly and the dry loaded crude mixture was purified by silica gel flash chromatography eluting from 0 to 60% EtOAc in heptanes to yield methyl 3-fluoro-5-methylisonicotinate (249 mg, 86% yield) as a white solid. LCMS ESI m/z 170.0 [M+H]⁺.

### Step 3

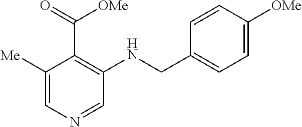 [SMILES: COC(=O)c1c(C)cncc1NCc1ccc(OC)cc1]

### Methyl 3-((4-methoxybenzyl)amino)-5-methylisonicotinate

To a solution of methyl 3-fluoro-5-methylnicotinate (217 mg, 1.28 mmol) in NMP (6.41 mL) was added N,N-diisopropylethylamine (677 μL, 3.85 mmol) and 4-Methoxybenzylamine (171 uL, 1.28 mmol). The reaction mixture was stirred at 80° C. for 16 h. A saturated solution of sodium bicarbonate was added to the reaction mixture and it was extracted 3 times with EtOAc. Organics were combined, washed with water, brine, dried with anhydrous sodium sulfate, filtered and concentrated to dryness. The crude residue was purified by flash chromatography on silica gel (50 g column, from 5 to 60% EtOAc in heptanes) to yield methyl 3-((4-methoxybenzyl)amino)-5-methylisonicotinate (12.6 mg, 3.4% yield) as a yellow oil. LCMS ESI m/z 287.0 [M+H]⁺.

### Step 4

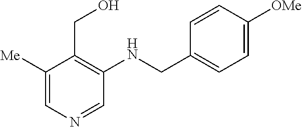 [SMILES: COc1ccc(CNc2cncc(C)c2CO)cc1]

### (3-((4-Methoxybenzyl)amino)-5-methylpyridin-4-yl)methanol

To a solution of methyl 3-((4-methoxybenzyl)amino)-5-methylisonicotinate (132 mg, 461 μmol) in THF (2.31 mL) was added a 2M solution of lithiumborohydride (692 μL, 1.38 mmol) and the reaction mixture was stirred at room temperature for 16 h. MeOH was added to the reaction mixture and it was stirred for 1 h and then concentrated to dryness to yield (3-((4-methoxybenzyl)amino)-5-methylpyridin-4-yl) methanol (119 mg, 100% yield) as a white solid. The product was carried over the next step without purification. LCMS ESI m/z 259.1 [M+H]⁺.

### Step 5

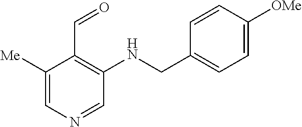 [SMILES: COc1ccc(CNc2cncc(C)c2C=O)cc1]

### 3-((4-Methoxybenzyl)amino)-5-methylisonicotinaldehyde

To a solution of (3-((4-methoxybenzyl)amino)-5-methylpyridin-4-yl)methanol (119 mg, 461 μmol) in DCM (2.30 mL) was added manganese (IV) oxide activated (222 mg, 2.30 mmol) and the reaction mixture was stirred at room temperature for 16 h. The reaction mixture was filtered through Celite and rinsed with DCM. The filtrate was concentrated to dryness. The crude residue was purified by flash chromatography on silica gel (25 g column, 10 to 80% EtOAc in heptanes) to yield 3-((4-Methoxybenzyl)amino)-5-methylisonicotinaldehyde (65.3 mg, 55% yield) as a yellow solid. LCMS ESI m/z 257.2 [M+H]⁺.

### Step 6

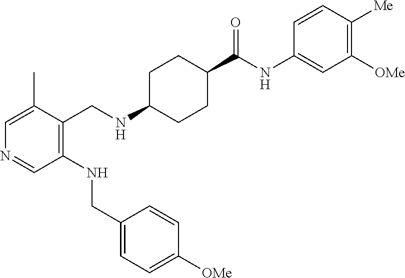 [SMILES: COc1ccc(CNc2cncc(C)c2CN[C@H]2CC[C@@H](C(=O)Nc3ccc(C)c(OC)c3)CC2)cc1]

### (1s,4s)-4-(((3-amino-5-methylpyridin-4-yl)methyl)amino)-N-(3-methoxy-4-methylphenyl)cyclohexanecarboxamide

3-((4-Methoxybenzyl)amino)-5-methylisonicotinaldehyde (65.0 mg, 254 μmol) and (1s,4s)-4-amino-N-(3-methoxy-4-methylphenyl)cyclohexanecarboxamide (67.2 mg, 256 μmol) were place in a vial and DCE (1.69 mL) was added followed by a drop of acetic acid. The reagents were mixed for 16 hours at 50° C. The reaction mixture was cooled to room temperature and Sodium triacetoxyborohydride (160 mg, 761 μmol) was added in one portion. The reaction mixture was stirred at 23° C. for 48 h. MeOH was added to the reaction mixture and the resulting solution was stirred at room temperature for 1 h and concentrated to dryness. The crude residue was purified by flash chromatography on silica gel (from 1 to 60% (20% MeOH in DCM) in DCM) to yield (1s,4s)-4-(((3-amino-5-methylpyridin-4-yl)methyl)amino)-N-(3-methoxy-4-methylphenyl)cyclohexanecarboxamide (54.7 mg, 43% yield) as a pale yellow solid. LCMS ESI m/z 503.2 [M+H]⁺.

### Step 7

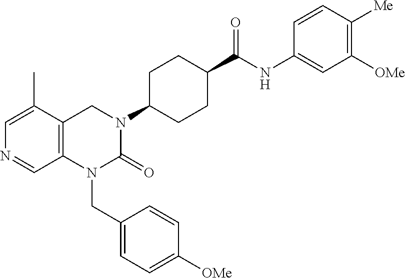 [SMILES: COc1ccc(CN2C(=O)N([C@H]3CC[C@@H](C(=O)Nc4ccc(C)c(OC)c4)CC3)Cc3c(C)cncc32)cc1]

### (1s,4s)-N-(3-Methoxy-4-methylphenyl)-4-(1-(4-methoxybenzyl)-5-methyl-2-oxo-1,2-dihydropyrido[3,4-d]pyrimidin-3(4H)-yl)cyclohexanecarboxamide

To a solution of (1s,4s)-4-(((3-amino-5-methylpyridin-4-yl)methyl)amino)-N-(3-methoxy-4-methylphenyl)cyclohexanecarboxamide (55.0 mg, 109 μmol) in DCM (547 μL) was added triphosgene (11.6 mg, 38.3 μmol) followed by triethylamine (30.7 μL, 219 μmol) and the reaction mixture was stirred at room temperature for 5 h. The reaction mixture was concentrated to dryness and the crude residue was purified by flash chromatography on silica gel (25 g column, from 1 to 60% (20% MeOH in DCM) in DCM) to yield (1s,4s)-N-(3-methoxy-4-methylphenyl)-4-(1-(4-methoxybenzyl)-5-methyl-2-oxo-1,2-dihydropyrido[3,4-d]pyrimidin-3(4H)-yl)cyclohexanecarboxamide (13.7 mg, 24% yield) as a colorless gum. LCMS ESI m/z 529.2 [M+H]⁺.

### Step 8

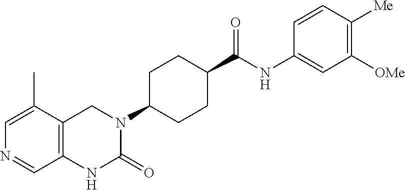 [SMILES: COc1cc(NC(=O)[C@H]2CC[C@@H](N3Cc4c(C)cncc4NC3=O)CC2)ccc1C]

### (1s,4s)-N-(3-Methoxy-4-methylphenyl)-4-(5-methyl-2-oxo-1,2-dihydropyrido[3,4-d]pyrimidin-3(4H)-yl)cyclohexanecarboxamide

To a solution of (1s,4s)-N-(3-methoxy-4-methylphenyl)-4-(1-(4-methoxybenzyl)-5-methyl-2-oxo-1,2-dihydropyrido[3,4-d]pyrimidin-3(4H)-yl)cyclohexanecarboxamide (13.7 mg, 25.9 μmol) in DCM (0.2 mL) was added trifluoroacetic acid (0.2 mL, 2.61 mmol). The reaction mixture was heated to 55° C. for 4 h. The reaction mixture was concentrated under reduced pressure and the crude residue was purified by reverse phase flash chromatography on C18 (30 g column, from 0 to 60% MeCN in water-10 mM ammonium formate pH=3.8). Pure fractions were directly lyophilized to yield (1s,4s)-N-(3-methoxy-4-methylphenyl)-4-(5-methyl-2-oxo-1,2-dihydropyrido[3,4-d]pyrimidin-3(4H)-yl)cyclohexanecarboxamide (1.58 mg, 15% yield) as a white solid. LCMS ESI m/z 409.9 [M+H]⁺. ¹H NMR (400 MHz, DMSO-d₆) δ 9.67 (s, 1H), 9.27 (s, 1H), 7.88 (s, 1H), 7.84 (s, 1H), 7.28 (d, J=1.5 Hz, 1H), 7.10 (dd, J=8.1, 1.7 Hz, 1H), 7.00 (d, J=8.2 Hz, 1H), 4.27 (s, 2H), 4.19 (tt, J=12.2, 3.6 Hz, 1H), 3.74 (s, 3H), 2.66-2.60 (m, 1H), 2.13 (s, 3H), 2.16-2.08 (m, 2H), 2.06 (s, 3H), 2.04-1.94 (m, 2H), 1.70-1.57 (m, 2H), 1.44 (dd, J=12.2, 3.1 Hz, 2H).

### Example 38

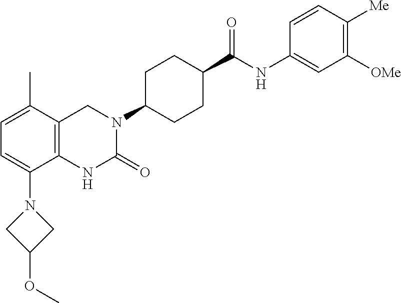 [SMILES: COc1cc(NC(=O)[C@H]2CC[C@@H](N3Cc4c(C)ccc(N5CC(OC)C5)c4NC3=O)CC2)ccc1C]

### (1s,4s)-N-(3-Methoxy-4-methylphenyl)-4-(8-(3-methoxyazetidin-1-yl)-5-methyl-2-oxo-1,2-dihydroquinazolin-3(4H)-yl)cyclohexanecarboxamide

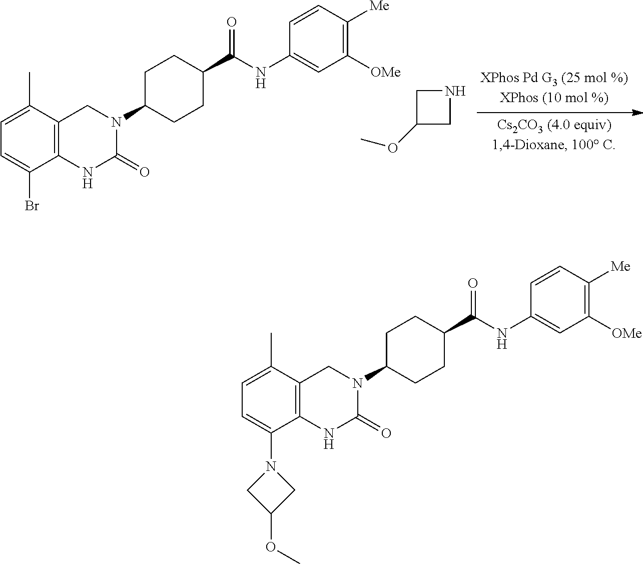

### Step 1

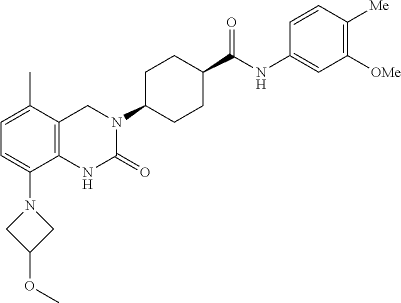 [SMILES: COc1cc(NC(=O)[C@H]2CC[C@@H](N3Cc4c(C)ccc(N5CC(OC)C5)c4NC3=O)CC2)ccc1C]

### (1s,4s)-N-(3-Methoxy-4-methylphenyl)-4-(8-(3-methoxyazetidin-1-yl)-5-methyl-2-oxo-1,2-dihydroquinazolin-3(4H)-yl)cyclohexanecarboxamide

To a flame-dried, nitrogen-flushed 0.5-2.0 mL Biotage vial was added (1s,4s)-4-(8-Bromo-5-methyl-2-oxo-1,2-dihydroquinazolin-3(4H)-yl)-N-(3-methoxy-4-methylphenyl)cyclohexanecarboxamide from Example 34 (25.0 mg, 51.4 μmol) and it was diluted with 1,4-Dioxane (257 mL). The solvent was sparged with nitrogen for 5 minutes. Then, XPhos Pd G3 (8.97 mg, 10.3 μmol), Cesium carbonate (76.9 mg, 231 μmol), 3-Methoxyazetidine hydrochloride (32.4 mg, 262 μmol) and 2-dicyclohexylphosphino-2′,4′,6′-triisopropylbiphenyl (2.53 mg, 5.14 μmol) were added in that order. The vial was sealed with a microwave cap and heated to 100° C. overnight. The reaction was then cooled to room temperature and opened to air. Solvent was evaporated to dryness. Purification of the crude was performed using Biotage purification system over 12 g (100% aqueous ammonium formate (AMF) solution (pH=3.8) to 70% MeCN in AMF. Lyophilization of the fraction provided (1s,4s)-N-(3-methoxy-4-methylphenyl)-4-(8-(3-methoxyazetidin-1-yl)-5-methyl-2-oxo-1,2-dihydroquinazolin-3(4H)-yl)cyclohexanecarboxamide as a white solid (3.30 mg, 12% yield) isolated. LCMS ESI m/z 493.3 [M+H]⁺. ¹H NMR (400 MHz, DMSO-d₆) δ 9.68 (s, 1H), 7.27 (s, 1H), 7.23 (s, 1H), 7.09 (d, J=8.1 Hz, 1H), 7.00 (d, J=8.1 Hz, 1H), 6.65 (d, J=8.3 Hz, 1H), 6.51 (d, J=8.3 Hz, 1H), 4.23-4.09 (m, 2H), 4.18 (s, 2H), 4.02 (dd, J=7.7, 6.2 Hz, 2H), 3.73 (s, 3H), 3.40 (dd, J=7.8, 5.4 Hz, 2H), 3.19 (s, 3H), 2.64-2.61 (m, 1H), 2.09-2.02 (m, 2H), 2.06 (s, 3H), 2.03 (s, 3H), 2.02-1.91 (m, 2H), 1.71-1.59 (m, 2H), 1.49-1.41 (m, 2H).

### Example 39

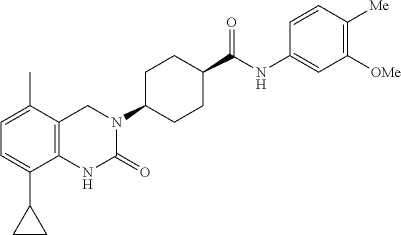 [SMILES: COc1cc(NC(=O)[C@H]2CC[C@@H](N3Cc4c(C)ccc(C5CC5)c4NC3=O)CC2)ccc1C]

### (1s,4s)-4-(8-Cyclopropyl-5-methyl-2-oxo-1,2-dihydroquinazolin-3(4H)-yl)-N-(3-methoxy-4-methylphenyl)cyclohexanecarboxamide

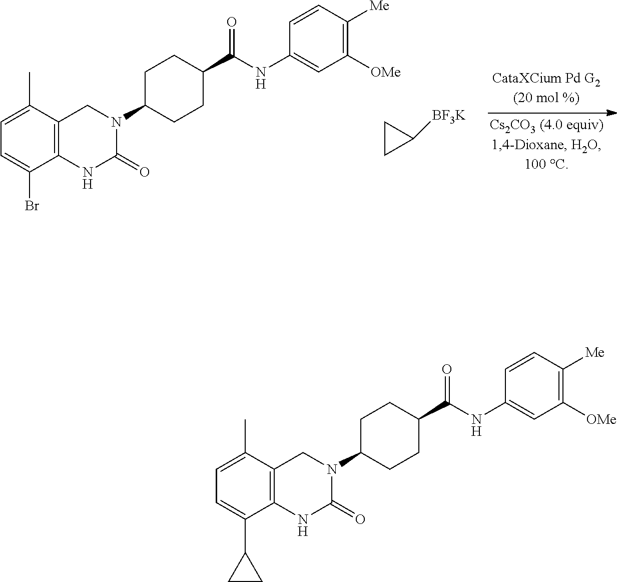

### Step 1

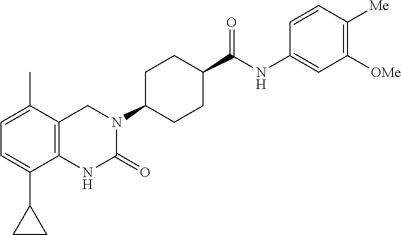 [SMILES: COc1cc(NC(=O)[C@H]2CC[C@@H](N3Cc4c(C)ccc(C5CC5)c4NC3=O)CC2)ccc1C]

### (1s,4s)-4-(8-Cyclopropyl-5-methyl-2-oxo-1,2-dihydroquinazolin-3(4H)-yl)-N-(3-methoxy-4-methylphenyl)cyclohexanecarboxamide

To a flame-dried, nitrogen-flushed 0.5-2.0 mL Biotage vial was added (1s,4s)-4-(8-Bromo-5-methyl-2-oxo-1,2-dihydroquinazolin-3(4H)-yl)-N-(3-methoxy-4-methylphenyl)cyclohexanecarboxamide from Example 34 (30.0 mg, 61.7 μmol) and it was diluted with 1,4-Dioxane (308 μL), H₂O (308 μL). The solvent was sparged with nitrogen for 5 minutes. Then, chloro [(di(1-adamantyl)-N-butylphosphine)-2-(2-aminobiphenyl)]palladium (II) (8.41 mg, 12.3 μmol), cesium carbonate (61.5 mg, 185 μmol), and potassium cyclopropyltrifluoroborate (45.6 mg, 308 μmol) were added in that order. The vial was sealed with a microwave cap and heated to 100° C. 4 hours. The reaction was then cooled to room temperature and opened to air. The reaction was stopped by evaporation to dryness. It resulted in a black residue which was purified by reverse chromatography over C18. Purification of the crude was performed using a C18 column. A gradient of 100% aqueous ammonium formate (AMF) solution (pH=3.8) to 90% MeCN in AMF was used. Lyophilization of the fraction provided (1s,4s)-4-(8-Cyclopropyl-5-methyl-2-oxo-1,2-dihydroquinazolin-3(4H)-yl)-N-(3-methoxy-4-methylphenyl)cyclohexanecarboxamide as a white solid (10.5 mg, 37% yield). LCMS ESI m/z 448.3 [M+H]⁺. ¹H NMR (400 MHz, DMSO-d₆) δ 9.67 (s, 1H), 8.06 (s, 1H), 7.27 (s, 1H), 7.09 (d, J=8.2 Hz, 1H), 7.00 (d, J=8.0 Hz, 1H), 6.72 (d, J=7.8 Hz, 1H), 6.62 (d, J=7.9 Hz, 1H), 4.26-4.14 (m, 1H), 4.20 (s, 2H), 3.74 (s, 3H), 2.68-2.59 (m, 1H), 2.17-2.04 (m, 2H), 2.11 (s, 3H), 2.07 (s, 3H), 2.04-1.95 (m, 2H), 1.90-1.81 (m, 1H), 1.73-1.61 (m, 2H), 1.50-1.42 (m, 2H), 0.88-0.81 (m, 2H), 0.49-0.43 (m, 2H).

### Example 40

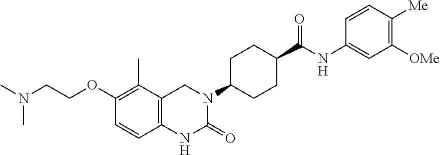 [SMILES: COc1cc(NC(=O)[C@H]2CC[C@@H](N3Cc4c(ccc(OCCN(C)C)c4C)NC3=O)CC2)ccc1C]

### (1s,4s)-4-(6-(2-(Dimethylamino)ethoxy)-5-methyl-2-oxo-1,2-dihydroquinazolin-3(4H)-yl)-N-(3-methoxy-4-methylphenyl)cyclohexanecarboxamide

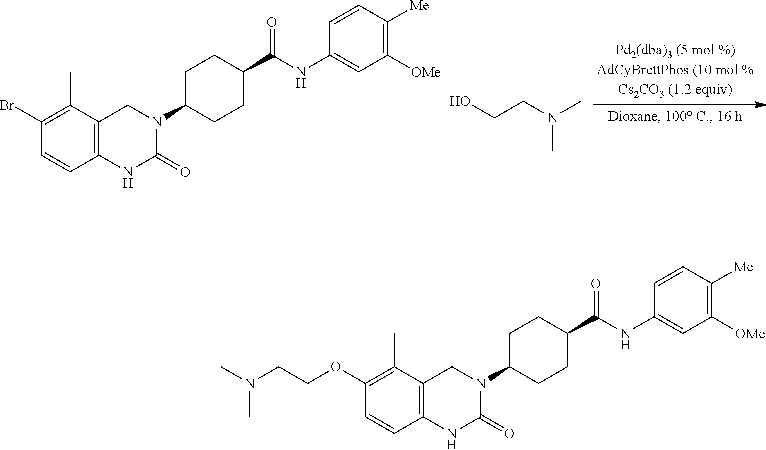

### Step 1

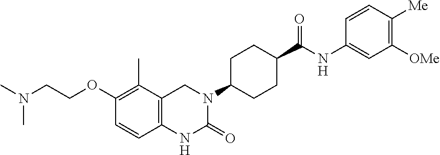 [SMILES: COc1cc(NC(=O)[C@H]2CC[C@@H](N3Cc4c(ccc(OCCN(C)C)c4C)NC3=O)CC2)ccc1C]

### (1s,4s)-4-(6-(2-(Dimethylamino)ethoxy)-5-methyl-2-oxo-1,2-dihydroquinazolin-3(4H)-yl)-N-(3-methoxy-4-methylphenyl)cyclohexanecarboxamide

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide from Example 63 (20.0 mg, 41.1 μmol) and it was diluted with anhydrous 1,4-Dioxane (206 μL). The solvent was sparged with nitrogen for 5 minutes. Then, 2-Dimethylaminoethanol (12.5 μL, 123 μmol), Cesium carbonate (41.0 mg, 123 μmol), AdCyBrettPhos (2.42 mg, 4.11 μmol), and Tris(dibenzylideneacetone)-dipalladium(0) (1.92 mg, 2.06 μmol) were added in that order. The vial was sealed with a microwave cap and heated to 100° C. for 3 hours. The reaction was then cooled to room temperature and opened to air. It was diluted with EtOAc, transferred to a 60 mL extraction funnel and diluted further with brine. The organics were washed with brine (3×20 mL), dries over Na₂SO₄, filtered and evaporated to dryness. It resulted in a black oil which was purified by reverse phase chromatography. Purification of the crude was performed using a C18 column. A gradient of 100% aqueous ammonium formate (AMF) solution (pH=3.8) to 70% MeCN in AMF was used. Evaporation and repurification with ammonium bicarbonate (AMB) instead of AMF under a similar gradient and lyophilization of purest fraction gave (1s,4s)-4-(6-(2-(Dimethylamino)ethoxy)-5-methyl-2-oxo-1,2-dihydroquinazolin-3(4H)-yl)-N-(3-methoxy-4-methylphenyl)cyclohexanecarboxamide as a white solid (3.60 mg, 6.9% yield). LCMS ESI m/z 495.2 [M+H]⁺. ¹H NMR (400 MHz, DMSO-d₆) δ 9.68 (s, 1H), 8.89 (s, 1H), 7.27 (d, J=1.6 Hz, 1H), 7.10 (dd, J=8.2, 1.8 Hz, 1H), 7.00 (d, J=8.2 Hz, 1H), 6.74 (d, J=8.5 Hz, 1H), 6.51 (d, J=8.8 Hz, 1H), 4.23-4.14 (m, 1H), 4.19 (s, 2H), 3.91 (t, J=5.6 Hz, 2H), 3.73 (s, 3H), 2.64-2.61 (m, 1H), 2.57 (t, J=5.6 Hz, 2H), 2.18 (s, 6H), 2.14-2.02 (m, 2H), 2.06 (s, 3H), 2.02-1.92 (m, 2H), 2.00 (s, 3H), 1.69-1.57 (m, 2H), 1.48-1.39 (m, 2H).

### Example 41

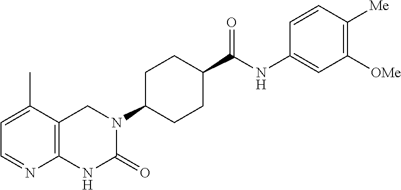 [SMILES: COc1cc(NC(=O)[C@H]2CC[C@@H](N3Cc4c(C)ccnc4NC3=O)CC2)ccc1C]

### (1s,4s)-N-(3-Methoxy-4-methylphenyl)-4-(5-methyl-2-oxo-1,2-dihydropyrido[2,3-d]pyrimidin-3(4H)-yl)cyclohexanecarboxamide

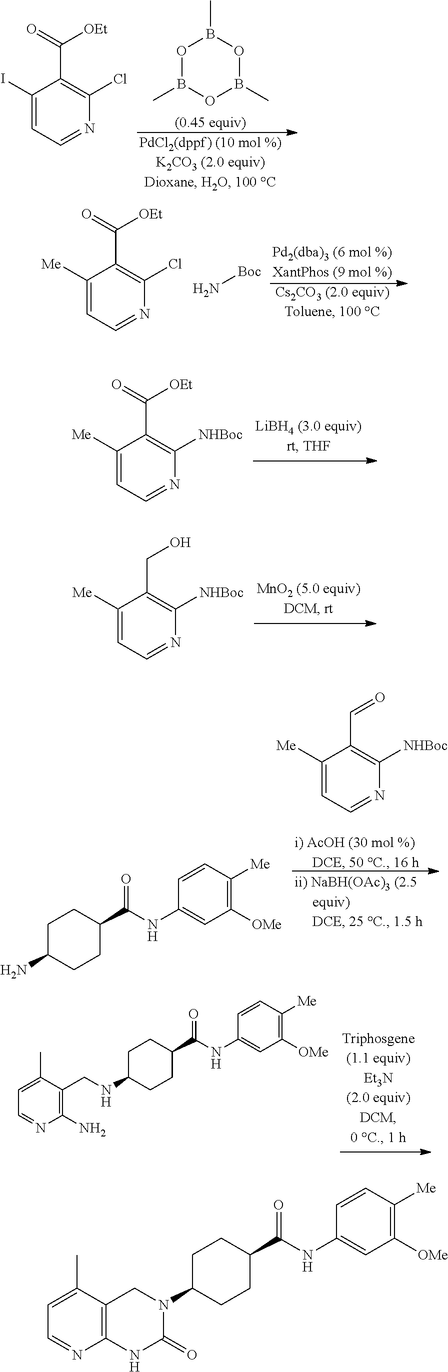

### Step 1

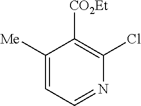 [SMILES: CCOC(=O)c1c(C)ccnc1Cl]

### Methyl 2-chloro-4-methylnicotinate

A microwave vial was charged with potassium carbonate (344 mg, 2.49 mmol), ethyl 2-chloro-4-iodonicotinate (388 mg, 1.25 mmol) and trimethylboroxine (79.1 uL, 561 μmol). Water (2.08 mL) and 1,4-dioxane (10.4 mL) were added, the vial was capped with a septum and the reaction mixture was degassed with a nitrogen flow for 10 minutes. Finally, 1,1′-Bis(diphenylphosphino)ferrocene dichloropalladium (II) (93.0 mg, 125 μmol) was then added to the vial under nitrogen and the reaction mixture was capped and stirred at 100° C. for 24 h. The reaction mixture was cooled to room temperature, silica gel was added directly and then dry loaded crude mixture was purified by silica gel flash chromatography eluting from 0 to 60% EtOAc in heptanes to yield methyl 2-chloro-4-methylnicotinate (186 mg, 75% yield) as a pale yellow oil. LCMS ESI m/z 200.0 [M+H]⁺.

### Step 2

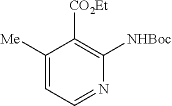 [SMILES: CCOC(=O)c1c(C)ccnc1NC(=O)OC(C)(C)C]

### Methyl 2-((tert-butoxycarbonyl)amino)-4-methylnicotinate

tert-Butyl carbamate (168 mg, 1.40 mmol), tris(dibenzylideneacetone)-dipalladium (0) (38.5 mg, 42.1 μmol), 4,4-bis(diphenylphosphino)-9,9-dimethylxanthene (37.3 mg, 63.1 μmol), Cesium carbonate (457 mg, 1.40 mmol) and ethyl 2-chloro-4-methylnicotinate (140 mg, 701 μmol) were placed in a microwave vial and capped under nitrogen (vacuum and backfilled with nitrogen). In the meantime, toluene was purged with nitrogen for 10 minutes. Toluene (4.68 mL) was then added to the reaction vessel and the reaction mixture was stirred under nitrogen at 100° C. for 5 h. The reaction mixture was cooled to room temperature and directly loaded to silica gel column and eluted from 0 to 50% EtOAc in heptane to yield methyl 2-chloro-4-methylnicotinate (105 mg, 53% yield) as a yellow solid. LCMS ESI m/z 222.0 [M+H]⁺.

### Step 3

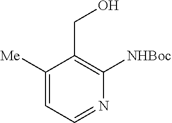 [SMILES: Cc1ccnc(NC(=O)OC(C)(C)C)c1CO]

### tert-Butyl (3-(hydroxymethyl)-4-methylpyridin-2-yl)carbamate

To a solution of methyl 2-((tert-butoxycarbonyl)amino)-4-methylnicotinate (79.4 mg, 478 μmol) in THF (2.39 mL) was added a 2M solution of Lithiumborohydride (717 μL, 1.43 mmol) in THF and the reaction mixture was stirred at room temperature for 16 h. A saturated solution of sodium bicarbonate was added to reaction mixture and it was extracted 3 times with EtOAc. Organics were combined, washed with brine, dried with anhydrous sodium sulfate, filtered and concentrated to dryness to yield tert-butyl (3-(hydroxymethyl)-4-methylpyridin-2-yl)carbamate (46.4 mg, 70% yield) brown gum which was used without purification in the next step. LCMS ESI m/z 238.9 [M+H]⁺.

### Step 4

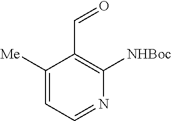 [SMILES: Cc1ccnc(NC(=O)OC(C)(C)C)c1C=O]

### tert-Butyl (3-formyl-4-methylpyridin-2-yl)carbamate

To a solution of tert-butyl (3-(hydroxymethyl)-4-methylpyridin-2-yl)carbamate (99.6 mg, 418 μmol) in DCM (2.09 mL) was added manganese (IV) oxide activated (202 mg, 2.09 mmol) and the reaction mixture was stirred at room temperature for 16 h. The reaction mixture was filtered through Celite and rinsed with DCM. The filtrate was concentrated to dryness to yield tert-Butyl (3-formyl-4-methylpyridin-2-yl) carbamate (70.5 mg, 71% yield) as a light yellow solid which was used without purification in the next step. LCMS ESI m/z 236.8 [M+H]⁺.

### Step 5

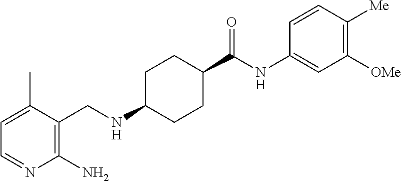 [SMILES: COc1cc(NC(=O)[C@H]2CC[C@@H](NCc3c(C)ccnc3N)CC2)ccc1C]

### (1s,4s)-4-(((2-Amino-4-methylpyridin-3-yl)methyl)amino)-N-(3-methoxy-4-methylphenyl)cyclohexanecarboxamide

tert-Butyl (3-formyl-4-methylpyridin-2-yl)carbamate (70.5 mg, 298 μmol) and (1s,4s)-4-amino-N-(3-methoxy-4-methylphenyl)cyclohexanecarboxamide (78.3 mg, 298 μmol) were place in a vial and DCE (1.99 mL) was added followed by a drop of acetic acid. The reagents were mixed for 16 hour at 50° C. Reaction mixture was cooled to room temperature and sodium triacetoxyborohydride (189 mg, 895 μmol) was added in one portion. The reaction mixture was stirred at 23° C. for 1 h. The reaction mixture was cooled to room temperature and MeOH was added, and the reaction mixture was concentrated to dryness. The crude residue was purified by flash chromatography on silica gel (25 g column, from 1 to 60% (20% MeOH/DCM) in DCM) to yield (1s,4s)-4-(((2-amino-4-methylpyridin-3-yl)methyl)amino)-N-(3-methoxy-4-methylphenyl)cyclohexanecarboxamide (74.3 mg, 65% yield) as a pale yellow gum. LCMS ESI m/z 383.2 [M+H]⁺.

### Step 6

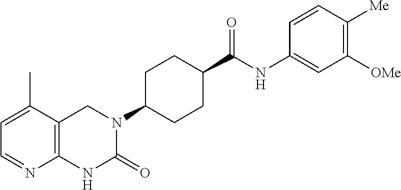 [SMILES: COc1cc(NC(=O)[C@H]2CC[C@@H](N3Cc4c(C)ccnc4NC3=O)CC2)ccc1C]

### (1s,4s)-N-(3-Methoxy-4-methylphenyl)-4-(5-methyl-2-oxo-1,2-dihydropyrido[2,3-d]pyrimidin-3(4H)-yl)cyclohexanecarboxamide

To a solution of (1s,4s)-4-(((2-amino-4-methylpyridin-3-yl)methyl)amino)-N-(3-methoxy-4-methylphenyl)cyclohexanecarboxamide (10.0 mg, 26.1 μmol) in DCM (131 μL) was added triphosgene (2.77 mg, 9.15 μmol) and triethylamine (7.32 μL, 52.3 μmol) and the reaction mixture was stirred at room temperature for 5 h. The reaction mixture was concentrated to dryness and the crude residue was purified by reverse phase flash chromatography on C18 (30 g column, from 0 to 60% MeCN in water-10 mM Ammonium formate pH=3.8). Pure fractions were directly lyophilized to yield (1s,4s)-N-(3-Methoxy-4-methylphenyl)-4-(5-methyl-2-oxo-1,2-dihydropyrido[2,3-d]pyrimidin-3(4H)-yl)cyclohexanecarboxamide (10.7 mg, 100% yield) white solid. LCMS ESI m/z 409.3 [M+H]⁺. ¹H NMR (400 MHz, DMSO-d₆) δ 9.69 (s, 1H), 9.43 (s, 1H), 7.88 (d, J=5.0 Hz, 1H), 7.27 (s, 1H), 7.10 (d, J=8.7 Hz, 1H), 7.00 (d, J=8.1 Hz, 1H), 6.73 (d, J=5.1 Hz, 1H), 4.25 (s, 2H), 4.23-4.14 (m, 1H), 3.73 (s, 3H), 2.67-2.60 (m, 1H), 2.15 (s, 3H), 2.11 (d, J=14.2 Hz, 2H), 2.06 (s, 3H), 2.02-1.92 (m, 2H), 1.70-1.58 (m, 2H), 1.45 (d, J=9.7 Hz, 2H).

### Example 42

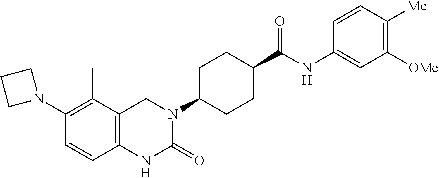 [SMILES: COc1cc(NC(=O)[C@H]2CC[C@@H](N3Cc4c(ccc(N5CCC5)c4C)NC3=O)CC2)ccc1C]

### (1s,4s)-4-(6-(Azetidin-1-yl)-5-methyl-2-oxo-1,2-dihydroquinazolin-3(4H)-yl)-N-(3-methoxy-4-methylphenyl)cyclohexanecarboxamide

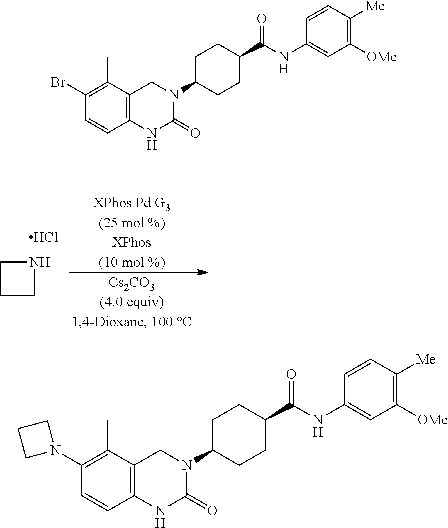

### Step 1

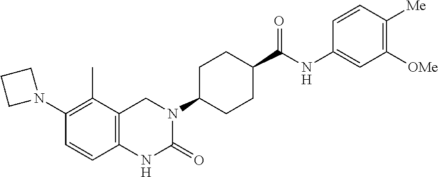 [SMILES: COc1cc(NC(=O)[C@H]2CC[C@@H](N3Cc4c(ccc(N5CCC5)c4C)NC3=O)CC2)ccc1C]

### (1s,4s)-4-(6-(Azetidin-1-yl)-5-methyl-2-oxo-1,2-dihydroquinazolin-3(4H)-yl)-N-(3-methoxy-4-methylphenyl)cyclohexanecarboxamide

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide from Example 63 (25.0 mg, 51.4 μmol) and it was diluted with 1,4-dioxane (257 mL). The solvent was sparged with nitrogen for 5 minutes. Then, XPhos Pd G3 (8.97 mg, 10.3 μmol), cesium carbonate (76.9 mg, 231 μmol), azetidine hydrochloride (15.2 mg, 154 μmol) and 2-dicyclohexylphosphino-2′,4′,6′-triisopropylbiphenyl (2.53 mg, 5.14 μmol) were added in that order. The vial was sealed with a microwave cap and heated to 100° C. overnight. The reaction was then cooled to room temperature and opened to air. Solvent was evaporated to dryness. Purification of the crude was performed using a gradient of 100% aqueous ammonium formate (AMF) solution (pH=3.8) to 70% MeCN in AMF was used. Lyophilization of the fraction provided (1s,4s)-4-(6-(Azetidin-1-yl)-5-methyl-2-oxo-1,2-dihydroquinazolin-3(4H)-yl)-N-(3-methoxy-4-methylphenyl)cyclohexanecarboxamide as a white solid (2.60 mg, 10% yield). LCMS ESI m/z 463.2 [M+H]⁺. ¹H NMR (400 MHz, dmso-d6) δ 9.68 (s, 1H), 8.81 (s, 1H), 7.27 (s, 1H), 7.10 (d, J=8.2 Hz, 1H), 7.00 (d, J=8.4 Hz, 1H), 6.48 (d, J=8.3 Hz, 1H), 6.36 (d, J=8.4 Hz, 1H), 4.22-4.11 (m, 1H), 4.17 (s, 2H), 3.73 (s, 3H), 3.65 (t, J=7.0 Hz, 4H), 2.65-2.60 (m, 1H), 2.15-2.08 (m, 4H), 2.03 (s, 3H), 2.04-1.94 (m, 2H), 1.94 (s, 3H), 1.69-1.56 (m, 2H), 1.46-1.37 (m, 2H).

### Example 43

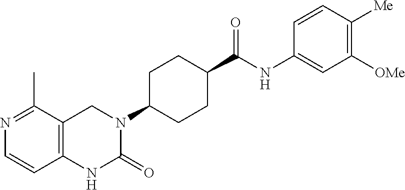 [SMILES: COc1cc(NC(=O)[C@H]2CC[C@@H](N3Cc4c(ccnc4C)NC3=O)CC2)ccc1C]

### (1s,4s)-N-(3-Methoxy-4-methylphenyl)-4-(5-methyl-2-oxo-1,2-dihydropyrido[4,3-d]pyrimidin-3(4H)-yl)cyclohexanecarboxamide

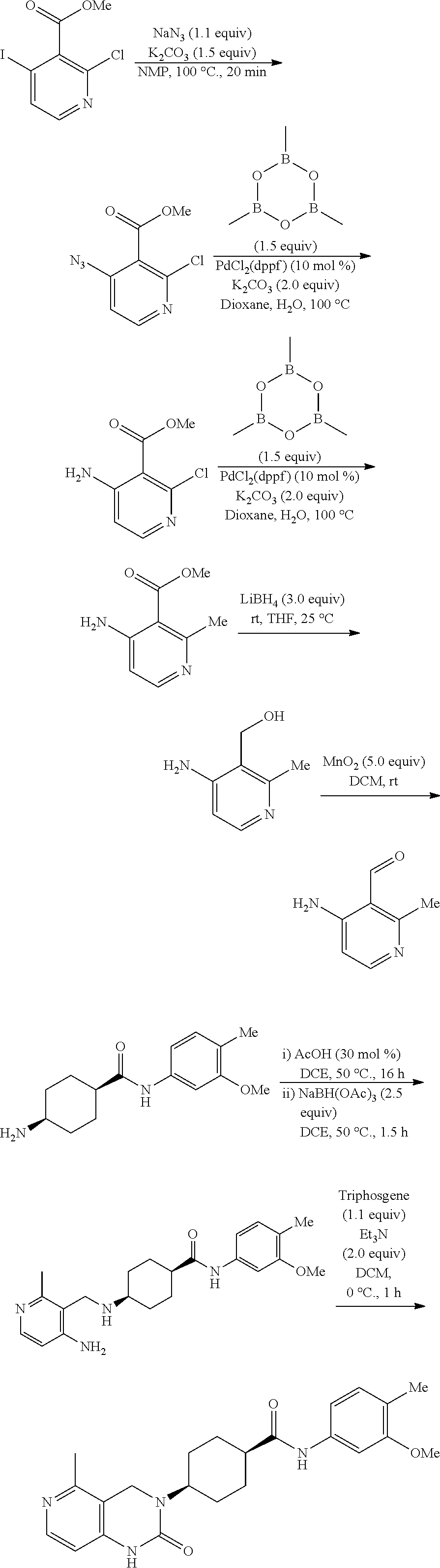

### Step 1

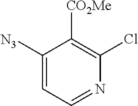 [SMILES: COC(=O)c1c(N=[N+]=[N-])ccnc1Cl]

### Methyl 4-azido-2-chloronicotinate

To a solution of methyl 2-chloro-4-iodonicotinate (448 mg, 1.51 mmol) in NMP (7.53 mL) was added potassium carbonate (312 mg, 2.26 mmol) and sodium azide (108 μL, 1.66 mmol) and the reaction mixture was heated at 100° C. and stirred for 20 minutes. A saturated solution of sodium bicarbonate was added to reaction mixture and it was extracted 3 times with EtOAc. Organics were combined, washed with brine, dried with anhydrous sodium sulfate, filtered and concentrated to dryness. The crude residue was purified by flash chromatography on silica gel (50 g column, from 2 to 60% EtOAc in heptanes) to yield methyl 4-azido-2-chloronicotinate (149 mg, 47% yield) as a white solid. LCMS ESI m/z 185.0 [M-N₂]⁺. ¹H NMR (400 MHz, CDCl₃) δ 8.38 (d, J=5.6 Hz, 1H), 7.07 (d, J=5.6 Hz, 1H), 3.97 (s, 3H).

### Step 2

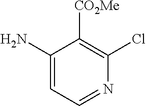 [SMILES: COC(=O)c1c(N)ccnc1Cl]

### Methyl 4-amino-2-chloronicotinate

A microwave vial was charged with potassium carbonate (194 mg, 1.40 mmol), methyl 4-azido-2-chloronicotinate (149 mg, 701 μmol) and trimethylboroxine (148 μL, 1.05 mmol). 1,4-Dioxane (5.84 mL) and water (1.17 mL) were added and the mixture was degassed for a 15 minutes period using a flow of nitrogen. Finally, 1,1′-bis(diphenylphosphino)ferrocene dichloropalladium (II) (52.3 mg, 70.1 μmol) was added under nitrogen, the vial was capped and the reaction mixture was stirred at 100° C. for 16 h. The reaction mixture was cold to room temperature, silica gel was added directly and the dry loaded crude mixture was purified by silica gel flash chromatography eluting from 0 to 60% (20% MeOH in DCM) in DCM to yield methyl 4-amino-2-chloronicotinate (104 mg, 79% yield) as a brownish solid. LCMS ESI m/z 186.9 [M+H]⁺.

### Step 3

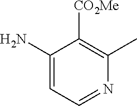

### Methyl 4-amino-2-methylnicotinate

A microwave vial was charged with, potassium carbonate (148 mg, 1.07 mmol), methyl 4-amino-2-chloronicotinate (100 mg, 536 μmol) and trimethylboroxine (114 uL, 804 μmol). 1,4-dioxane (4.47 mL) and water (893 uL) were added and the mixture was degassed for a 15 minutes period using a flow of nitrogen. Finally, 1,1′-Bis(diphenylphosphino)ferrocene dichloropalladium (II) (40.0 mg, 53.6 μmol) was added under nitrogen, the vial was capped and the reaction mixture was stirred at 100° C. for 16 h. The reaction mixture was cold to room temperature, silica gel was added directly and the dry loaded crude mixture was purified by silica gel flash chromatography eluting from 0 to 60% (20% MeOH in DCM) in DCM to yield methyl 4-amino-2-methylnicotinate (79.4 mg, 89% yield) as a brown solid. LCMS ESI m/z 167.1 [M+H]⁺.

### Step 4

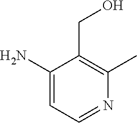

### (4-Amino-2-methylpyridin-3-yl)methanol

To a solution of methyl 4-amino-2-methylnicotinate (79.4 mg, 478 μmol) in THF (2.39 mL) was added a 2M solution of lithiumborohydride (717 μL, 1.43 mmol) in THF and the reaction mixture was stirred at room temperature for 16 h. A saturated solution of sodium bicarbonate was added to reaction mixture and it was extracted 3 times with EtOAc. Organics were combined, washed with brine, dried with anhydrous sodium sulfate, filtered and concentrated to dryness to yield crude methyl 4-amino-2-methylnicotinate (46.4 mg, 70% yield) brown gum used without purification. LCMS ESI m/z 138.9 [M+H]⁺.

### Step 5

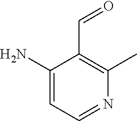

### 4-Amino-2-methylnicotinaldehyde

To a solution of (4-amino-2-methylpyridin-3-yl)methanol (46.4 mg, 336 μmol) in DCM (1.68 mL) was added Manganese (IV) oxide activated (162 mg, 1.68 mmol) and the reaction mixture was stirred at room temperature for 16 h. The reaction mixture was filtered through Celite and rinsed with DCM. The filtrate was concentrated to dryness to yield 4-amino-2-methylnicotinaldehyde (16.1 mg, 35% yield) as a light brown oil; no purification, use as crude for next step. LCMS ESI m/z 137.0 [M+H]⁺.

### Step 6

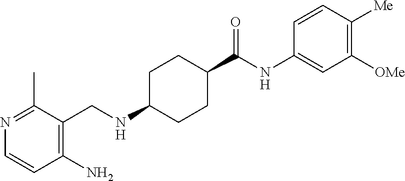 [SMILES: COc1cc(NC(=O)[C@H]2CC[C@@H](NCc3c(N)ccnc3C)CC2)ccc1C]

### (1s,4s)-4-(((4-Amino-2-methylpyridin-3-yl)methyl)amino)-N-(3-methoxy-4-methylphenyl)cyclohexanecarboxamide

4-Amino-2-methylnicotinaldehyde (16.0 mg, 118 μmol) and (1s,4s)-4-amino-N-(3-methoxy-4-methylphenyl)cyclohexanecarboxamide (30.8 mg, 118 μmol) were place in a vial and DCE (783 μL) was added followed by a drop of acetic acid. The reagents were mixed for 16 hour at 50° C. Sodium triacetoxyborohydride (74.4 mg, 353 μmol) was added in one portion. The reaction mixture was stirred at 50° C. for 1 h. The reaction mixture was cooled to room temperature and MeOH was added. The solution was concentrated to dryness and the crude residue was purified by reverse phase flash chromatography on C18 (30 g column, 0 to 60% MeCN in water-10 mM Ammonium formate pH=3.8). Pure fractions were concentrated to dryness to yield (1s,4s)-4-(((4-Amino-2-methylpyridin-3-yl)methyl)amino)-N-(3-methoxy-4-methylphenyl)cyclohexanecarboxamide (20.7 mg, 46% yield) as a white solid. LCMS ESI m/z 383.1 [M+H]⁺.

### Step 7

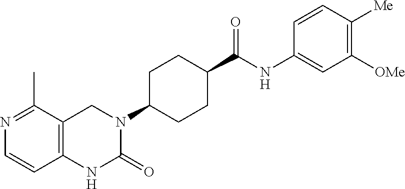 [SMILES: COc1cc(NC(=O)[C@H]2CC[C@@H](N3Cc4c(ccnc4C)NC3=O)CC2)ccc1C]

### (1s,4s)-N-(3-methoxy-4-methylphenyl)-4-(5-methyl-2-oxo-1,2-dihydropyrido[4,3-d]pyrimidin-3(4H)-yl)cyclohexanecarboxamide

To a suspension of (1s,4s)-4-(((4-amino-2-methylpyridin-3-yl)methyl)amino)-N-(3-methoxy-4-methylphenyl)cyclohexanecarboxamide (20.7 mg, 54.1 μmol) in DCM (1 mL) was added triphosgene (5.74 mg, 18.9 μmol) followed by dropwise addition of a solution of triethylamine (15.2 μL, 108 μmol) in 0.5 mL of DCM. The reaction mixture was stirred at room temperature for 4 h. After complete conversion of the starting material the reaction mixture was concentrated to dryness and the residual white solid was purified by reverse phase flash chromatography on C18 (30 g column, from 0 to 60% MeCN in water-10 mM Ammonium formate pH=3.8). Purest fractions were directly lyophilized to yield (1s,4s)-N-(3-methoxy-4-methylphenyl)-4-(5-methyl-2-oxo-1,2-dihydropyrido[4,3-d]pyrimidin-3(4H)-yl)cyclohexanecarboxamide (10.7 mg, 48% yield) as a white solid; formate salt. LCMS ESI m/z 409.3 [M+H]⁺. ¹H NMR (400 MHz, DMSO-d₆) δ 9.69 (s, 1H), 9.47 (s, 1H), 8.18 (s, 1H), 8.02 (d, J=5.4 Hz, 1H), 7.28 (d, J=1.6 Hz, 1H), 7.10 (dd, J=8.1, 1.8 Hz, 1H), 7.00 (d, J=8.3 Hz, 1H), 6.51 (d, J=5.4 Hz, 1H), 4.26 (s, 2H), 4.23-4.13 (m, 1H), 3.73 (s, 3H), 2.62-2.57 (m, 1H), 2.27 (s, 3H), 2.11 (d, J=13.2 Hz, 2H), 2.06 (s, 3H), 2.03-1.93 (m, 2H), 1.70-1.58 (m, 2H), 1.45 (dd, J=12.1, 3.2 Hz, 2H).

### Example 44

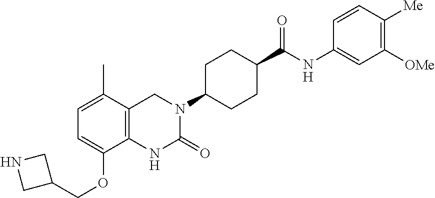 [SMILES: COc1cc(NC(=O)[C@H]2CC[C@@H](N3Cc4c(C)ccc(OCC5CNC5)c4NC3=O)CC2)ccc1C]

### (1s,4s)-4-(8-(Azetidin-3-ylmethoxy)-5-methyl-2-oxo-1,2-dihydroquinazolin-3(4H)-yl)-N-(3-methoxy-4-methylphenyl)cyclohexanecarboxamide

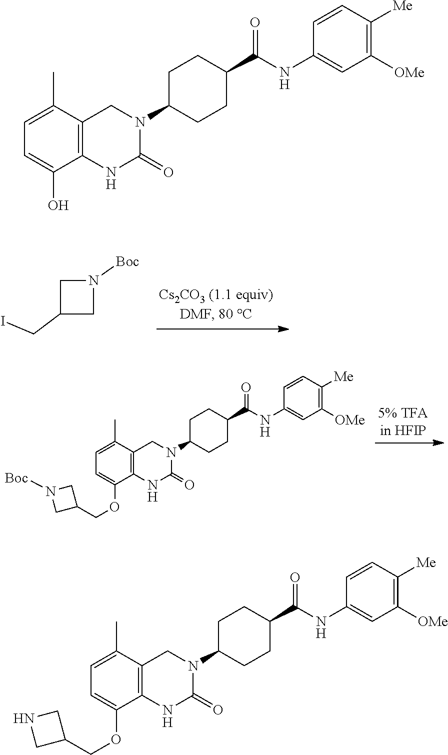 [SMILES: COc1cc(NC(=O)[C@H]2CC[C@@H](N3Cc4c(C)ccc(O)c4NC3=O)CC2)ccc1C ; CC(C)(C)OC(=O)N1CC(CI)C1 ; COc1cc(NC(=O)[C@H]2CC[C@@H](N3Cc4c(C)ccc(OCC5CN(C(=O)OC(C)(C)C)C5)c4NC3=O)CC2)ccc1C ; COc1cc(NC(=O)[C@H]2CC[C@@H](N3Cc4c(C)ccc(OCC5CNC5)c4NC3=O)CC2)ccc1C]

### Step 1

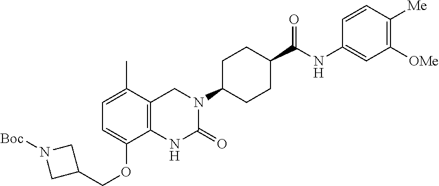 [SMILES: COc1cc(NC(=O)[C@H]2CC[C@@H](N3Cc4c(C)ccc(OCC5CN(C(=O)OC(C)(C)C)C5)c4NC3=O)CC2)ccc1C]

### tert-Butyl 3-(((3-((1s,4s)-4-((3-Methoxy-4-methylphenyl)carbamoyl)cyclohexyl)-5-methyl-2-oxo-1,2,3,4-tetrahydroquinazolin-8-yl)oxy)methyl)azetidine-1-carboxylate

To flame-dried, nitrogen-flushed 0.5-2.0 mL Biotage vial was added (1s,4s)-4-(8-hydroxy-5-methyl-2-oxo-1,2-dihydroquinazolin-3(4H)-yl)-N-(3-methoxy-4-methylphenyl)cyclohexanecarboxamide from Example 61 (15.0 mg, 17.7 μmol) and it was diluted with anhydrous DMF (177 μL)). The solvent was sparged with nitrogen for 5 minutes. Then, tert-Butyl 3-(iodomethyl)azetidine-1-carboxylate (7.00 μL, 53.7 μmol) and Cesium carbonate (6.48 mg, 19.5 μmol) were added in that order. The vial was sealed with a microwave cap and heated to 80° C. for 2 hours. After 2 hours, the reaction was then cooled to room temperature and opened to air. It resulted in a black residue which was purified by reverse phase chromatography over C18 (12 g SNAP Ultra, 100% ammonium formate solution, AMF, pH=3.8 to 90% MeCN in 10% AMF). The fractions containing desired product were combined and evaporated to dryness to provide tert-Butyl 3-(((3-((1s,4s)-4-((3-Methoxy-4-methylphenyl)carbamoyl)cyclohexyl)-5-methyl-2-oxo-1,2,3,4-tetrahydroquinazolin-8-yl)oxy)methyl)azetidine-1-carboxylate (6.50 mg, 62% yield) was isolated as a beige solid. LCMS ESI m/z 593.2 [M+H]⁺.

### Step 2

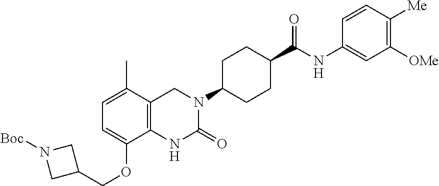 [SMILES: COc1cc(NC(=O)[C@H]2CC[C@@H](N3Cc4c(C)ccc(OCC5CN(C(=O)OC(C)(C)C)C5)c4NC3=O)CC2)ccc1C]

### (1,4s)-4-(8-(Azetidin-3-ylmethoxy)-5-methyl-2-oxo-1,2-dihydroquinazolin-3(4H)-yl)-N-(3-methoxy-4-methylphenyl)cyclohexanecarboxamide

To a 20 mL vial which contained tert-Butyl 3-(((3-((1s,4s)-4-((3-Methoxy-4-methylphenyl)carbamoyl)cyclohexyl)-5-methyl-2-oxo-1,2,3,4-tetrahydroquinazolin-8-yl)oxy)methyl)azetidine-1-carboxylate (6.50 mg, 11.0 μmol) was added a solution of 5% TFA in HFIP (2 mL) dropwise and reaction stirred for 1 hour. The crude was then evaporated to dryness to provide a translucent oil. Purification of the crude was performed using Biotage purification system over 12 g C18 column. A gradient of 100% aqueous ammonium formate (AMF) solution (pH=3.8) to 70% MeCN in AMF was used. Lyophilization of the fraction provided (1s,4s)-4-(8-(Azetidin-3-ylmethoxy)-5-methyl-2-oxo-1,2-dihydroquinazolin-3(4H)-yl)-N-(3-methoxy-4-methylphenyl)cyclohexanecarboxamide as a white solid (5.20 mg, 82% yield) isolated as a formate salt. LCMS ESI m/z 493.3 [M+H]⁺. ¹H NMR (400 MHz, DMSO-d₆) δ 9.71 (s, 1H), 8.39 (s, 1H), 8.23 (s, 1H), 7.27 (s, 1H), 7.09 (d, J=7.8 Hz, 1H), 7.01 (d, J=8.1 Hz, 1H), 6.75 (d, J=8.4 Hz, 1H), 6.67 (d, J=8.3 Hz, 1H), 4.29-4.13 (m, 1H), 4.21 (s, 2H), 4.02 (d, J=4.2 Hz, 2H), 3.91 (t, J=9.1 Hz, 2H), 3.79-3.69 (m, 2H), 3.73 (s, 3H), 3.16-3.08 (m, 1H), 2.67-2.60 (m, 1H), 2.16-2.03 (m, 2H), 2.10 (s, 3H), 2.06 (s, 3H), 2.01-1.93 (m, 2H), 1.72-1.60 (m, 2H), 1.48-1.40 (m, 2H).

### Example 45

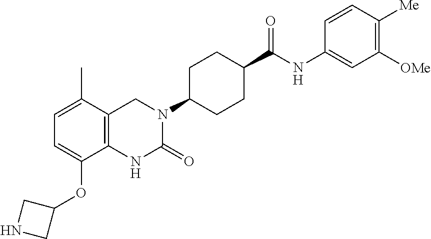 [SMILES: COc1cc(NC(=O)[C@H]2CC[C@@H](N3Cc4c(C)ccc(OC5CNC5)c4NC3=O)CC2)ccc1C]

### ((1s,4s)-4-(8-(Azetidin-3-yloxy)-5-methyl-2-oxo-1,2-dihydroquinazolin-3(4H)-yl)-N-(3-methoxy-4-methylphenyl)cyclohexanecarboxamide

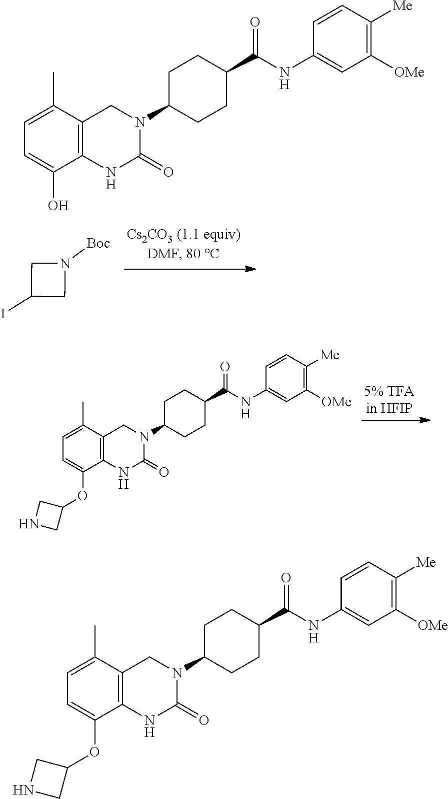 [SMILES: COc1cc(NC(=O)[C@H]2CC[C@@H](N3Cc4c(C)ccc(O)c4NC3=O)CC2)ccc1C ; CC(C)(C)OC(=O)N1CC(I)C1 ; COc1cc(NC(=O)[C@H]2CC[C@@H](N3Cc4c(C)ccc(OC5CNC5)c4NC3=O)CC2)ccc1C ; COc1cc(NC(=O)[C@H]2CC[C@@H](N3Cc4c(C)ccc(OC5CNC5)c4NC3=O)CC2)ccc1C]

### Step 1

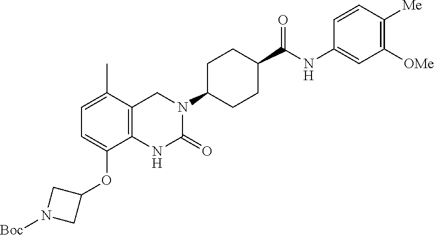 [SMILES: COc1cc(NC(=O)[C@H]2CC[C@@H](N3Cc4c(C)ccc(OC5CN(C(=O)OC(C)(C)C)C5)c4NC3=O)CC2)ccc1C]

### tert-Butyl 3-((3-((1s,4s)-4-((3-methoxy-4-methylphenyl)carbamoyl)cyclohexyl)-5-methyl-2-oxo-1,2,3,4-tetrahydroquinazolin-8-yl)oxy)azetidine-1-carboxylate

To flame-dried, nitrogen-flushed 0.5-2.0 mL Biotage vial was added (1s,4s)-4-(8-hydroxy-5-methyl-2-oxo-1,2-dihydroquinazolin-3(4H)-yl)-N-(3-methoxy-4-methylphenyl)cyclohexanecarboxamide from Example 61 (15.0 mg, 35.4 μmol) and it was diluted with DMF (177 μL). The solvent was sparged with nitrogen for 5 minutes. Then, N-Boc-3-iodoazetidine (13.3 μL, 107 μmol), cesium carbonate (13.0 mg, 39.0 μmol) were added in that order. The vial was sealed with a microwave cap and heated to 100° C. overnight. More cesium carbonate (13.0 mg, 39.0 μmol) was added. After another 30 minutes, the reaction was then cooled to room temperature and opened to air. It resulted in a black residue which was purified by reverse phase chromatography over C18 (12 g, 70% ammonium formate solution in 30% MeCN, AMF, pH=3.8 to 100% MeCN in 30% AMF). The fractions containing desired product were combined and evaporated to dryness. tert-Butyl 3-((3-((1s,4s)-4-((3-methoxy-4-methylphenyl)carbamoyl)cyclohexyl)-5-methyl-2-oxo-1,2,3,4-tetrahydroquinazolin-8-yl)oxy)azetidine-1-carboxylate (13.5 mg, 66% yield) was isolated as a beige solid. Carried over the next step (deprotection). LCMS ESI m/z 479.2 [M-Boc]⁺.

### Step 2

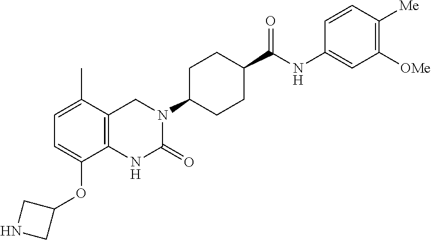 [SMILES: COc1cc(NC(=O)[C@H]2CC[C@@H](N3Cc4c(C)ccc(OC5CNC5)c4NC3=O)CC2)ccc1C]

### ((1s,4s)-4-(8-(Azetidin-3-yloxy)-5-methyl-2-oxo-1,2-dihydroquinazolin-3(4H)-yl)-N-(3-methoxy-4-methylphenyl)cyclohexanecarboxamide

To a 20 mL vial which contained tert-butyl 3-((3-((1s,4s)-4-((3-methoxy-4-methylphenyl)carbamoyl)cyclohexyl)-5-methyl-2-oxo-1,2,3,4-tetrahydroquinazolin-8-yl)oxy)azetidine-1-carboxylate (13.5 mg, 23.3 μmol) was added a solution of 5% TFA in HFIP (2 mL) dropwise and reaction stirred for 1 hour. The crude was then evaporated to dryness to provide a translucent oil. Purification of the crude with a gradient of 100% aqueous ammonium formate (AMF) solution (pH=3.8) to 70% MeCN in AMF was used. Lyophilization of the fraction provided desired product as a white solid. ((1s,4s)-4-(8-(Azetidin-3-yloxy)-5-methyl-2-oxo-1,2-dihydroquinazolin-3(4H)-yl)-N-(3-methoxy-4-methylphenyl)cyclohexanecarboxamide (6.50 mg, 51% yield) isolated as a white solid and as a formate salt. LCMS ESI m/z 479.3 [M+H]⁺. ¹H NMR (400 MHz, DMSO-d₆) δ 9.70 (s, 1H), 8.29 (s, 1H), 8.10 (s, 1H), 7.27 (d, J=1.7 Hz, 1H), 7.10 (dd, J=8.1, 1.8 Hz, 1H), 7.00 (d, J=8.5 Hz, 1H), 6.62 (d, J=8.5 Hz, 1H), 6.48 (d, J=8.3 Hz, 1H), 4.98-4.86 (m, 1H), 4.28-4.15 (m, 1H), 4.21 (s, 2H), 3.98-3.86 (m, 2H), 3.79-3.67 (m, 2H), 3.73 (s, 3H), 3.55-3.19 (m, 1H), 2.67-2.60 (m, 1H), 2.15-2.03 (m, 2H), 2.09 (s, 3H), 2.06 (s, 3H), 2.02-1.93 (m, 2H), 1.71-1.59 (m, 2H), 1.49-1.40 (m, 2H)

### Example 46

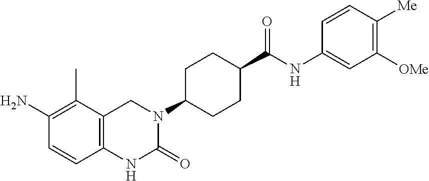 [SMILES: COc1cc(NC(=O)[C@H]2CC[C@@H](N3Cc4c(ccc(N)c4C)NC3=O)CC2)ccc1C]

### (1s,4s)-4-(6-Amino-5-methyl-2-oxo-1,2-dihydroquinazolin-3(4H)-yl)-N-(3-methoxy-4-methylphenyl)cyclohexanecarboxamide

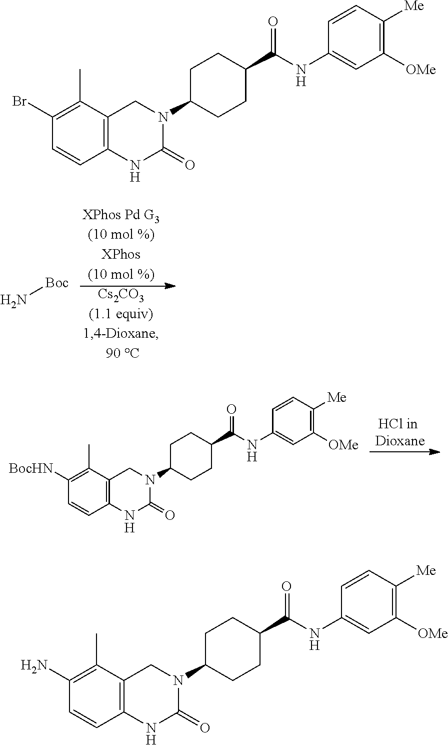 [SMILES: COc1cc(NC(=O)[C@H]2CC[C@@H](N3Cc4c(ccc(Br)c4C)NC3=O)CC2)ccc1C ; CC(C)(C)OC(N)=O ; COc1cc(NC(=O)[C@H]2CC[C@@H](N3Cc4c(ccc(NC(=O)OC(C)(C)C)c4C)NC3=O)CC2)ccc1C ; COc1cc(NC(=O)[C@H]2CC[C@@H](N3Cc4c(ccc(N)c4C)NC3=O)CC2)ccc1C]

### Step 1

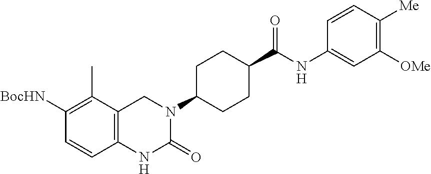 [SMILES: COc1cc(NC(=O)[C@H]2CC[C@@H](N3Cc4c(ccc(NC(=O)OC(C)(C)C)c4C)NC3=O)CC2)ccc1C]

### tert-Butyl (3-((1s,4s)-4-((3-methoxy-4-methylphenyl)carbamoyl)cyclohexyl)-5-methyl-2-oxo-1,2,3,4-tetrahydroquinazolin-6-yl)carbamate

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide from Example 63 (50.0 mg, 103 μmol) and it was diluted with 1,4-Dioxane (514 mL). The solvent was sparged with nitrogen for 5 minutes. Then, tert-Butyl carbamate (24.6 mg, 206 μmol) and 2-dicyclohexylphosphino-2′,4′,6′-triisopropylbiphenyl (5.05 mg, 10.3 μmol), cesium carbonate (37.6 mg, 113 μmol) and XPhos Pd G3 (8.97 mg, 10.3 μmol) were added in that order. The vial was sealed with a microwave cap and heated to 100° C. overnight. The reaction was then cooled to room temperature and opened to air. The crude suspension was directly subjected to deprotection. LCMS ESI m/z 523.3 [M+H]⁺.

### Step 2

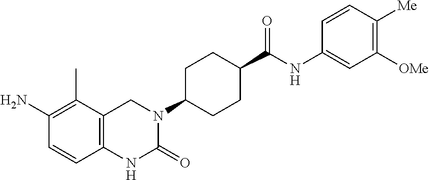 [SMILES: COc1cc(NC(=O)[C@H]2CC[C@@H](N3Cc4c(ccc(N)c4C)NC3=O)CC2)ccc1C]

### (1s,4s)-4-(6-Amino-5-methyl-2-oxo-1,2-dihydroquinazolin-3(4H)-yl)-N-(3-methoxy-4-methylphenyl)cyclohexanecarboxamide

To the crude mixture of tert-Butyl (3-((1s,4s)-4-((3-methoxy-4-methylphenyl)carbamoyl)cyclohexyl)-5-methyl-2-oxo-1,2,3,4-tetrahydroquinazolin-6-yl)carbamate (53.7 mg, 103 μmol) was added directly an excess amount of Hydrochloric acid in Dioxane (257 uL, 1.03 mmol). The mixture was stirred at room temperature for 1 hour. The reaction was evaporated to dryness. Purification of the crude was performed using a gradient of 100% aqueous ammonium bicarbonate (AMB) solution (pH=10.0) to 80% MeCN in AMB was used. Lyophilization of the fraction provided (1s,4s)-4-(6-Amino-5-methyl-2-oxo-1,2-dihydroquinazolin-3(4H)-yl)-N-(3-methoxy-4-methylphenyl)cyclohexanecarboxamide as a white solid (26.0 mg, 59% yield) over 2 steps. LCMS ESI m/z 423.3 [M+H]⁺. ¹H NMR (400 MHz, DMSO-d₆) δ 9.69 (s, 1H), 8.64 (s, 1H), 7.28 (s, 1H), 7.10 (d, J=8.4 Hz, 1H), 7.01 (d, J=7.6 Hz, 1H), 6.42 (d, J=8.3 Hz, 1H), 6.33 (d, J=8.5 Hz, 1H), 4.41 (s, 2H), 4.24-4.13 (m, 1H), 4.16 (s, 2H), 3.74 (s, 3H), 2.66-2.60 (m, 1H), 2.16-1.96 (m, 4H), 2.07 (s, 3H), 1.88 (s, 3H), 1.68-1.56 (m, 2H), 1.43-1.40 (m, 2H).

### Example 47

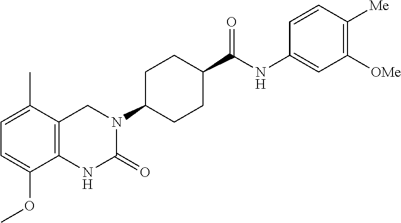 [SMILES: COc1cc(NC(=O)[C@H]2CC[C@@H](N3Cc4c(C)ccc(OC)c4NC3=O)CC2)ccc1C]

### (1s,4s)-N-(3-Methoxy-4-methylphenyl)-4-(8-methoxy-5-methyl-2-oxo-1,2-dihydroquinazolin-3(4H)-yl)cyclohexanecarboxamide

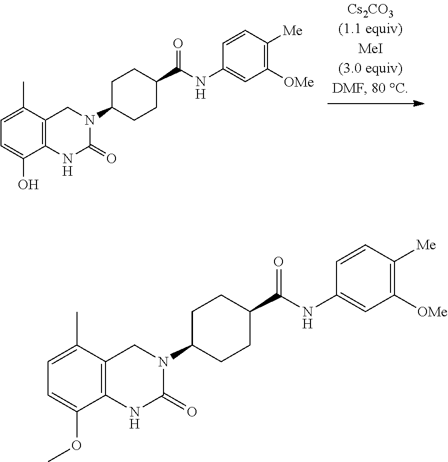 [SMILES: COc1cc(NC(=O)[C@H]2CC[C@@H](N3Cc4c(C)ccc(O)c4NC3=O)CC2)ccc1C ; COc1cc(NC(=O)[C@H]2CC[C@@H](N3Cc4c(C)ccc(OC)c4NC3=O)CC2)ccc1C]

### Step 1

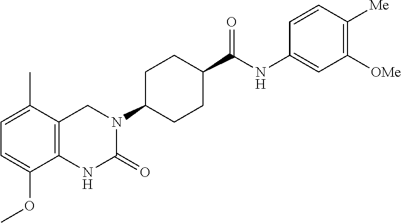 [SMILES: COc1cc(NC(=O)[C@H]2CC[C@@H](N3Cc4c(C)ccc(OC)c4NC3=O)CC2)ccc1C]

### (1s,4s)-N-(3-Methoxy-4-methylphenyl)-4-(8-methoxy-5-methyl-2-oxo-1,2-dihydroquinazolin-3(4H)-yl)cyclohexanecarboxamide

To a flame-dried, nitrogen-flushed 0.5-2.0 mL Biotage vial was added 1S,4S)-4-(8-hydroxy-5-methyl-2-oxo-1,2-dihydroquinazolin-3(4H)-yl)-N-(3-methoxy-4-methylphenyl)cyclohexanecarboxamide from Example 61 (15.0 mg, 35.4 μmol) and it was diluted with anhydrous DMF (177 uL). The solvent was sparged with nitrogen for 5 minutes. Then, iodomethane (6.68 μL, 106 μmol) and cesium carbonate (13.0 mg, 39.0 μmol) were added in that order. The vial was sealed with a microwave cap and heated to 80° C. for 2 hours. The reaction was then cooled to room temperature and opened to air. It resulted in a brown residue which was purified by reverse phase chromatography over C18 (12 g, 100% ammonium formate solution, AMF, pH=3.8 to 70% MeCN in 30% AMF). The fractions containing desired product were combined and lyophilized overnight. (1s,4s)-N-(3-Methoxy-4-methylphenyl)-4-(8-methoxy-5-methyl-2-oxo-1,2-dihydroquinazolin-3(4H)-yl)cyclohexanecarboxamide (6.30 mg, 38% yield) was isolated as a white solid. LCMS ESI m/z 438.2 [M+H]⁺. ¹H NMR (400 MHz, DMSO-d₆) δ 9.69 (s, 1H), 7.73 (s, 1H), 7.27 (s, 1H), 7.09 (d, J=8.4 Hz, 1H), 7.00 (d, J=8.1 Hz, 1H), 6.74 (d, J=8.3 Hz, 1H), 6.67 (d, J=8.0 Hz, 1H), 4.27-4.07 (m, 1H), 4.20 (s, 2H), 3.73 (s, 3H), 3.72 (s, 3H), 2.67-2.61 (m, 1H), 2.15-2.03 (m, 2H), 2.09 (s, 3H), 2.06 (s, 3H), 2.03-1.93 (m, 2H), 1.71-1.59 (m, 2H), 1.49-1.39 (m, 2H).

### Example 48

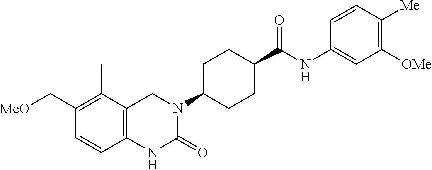 [SMILES: COCc1ccc2c(c1C)CN([C@H]1CC[C@@H](C(=O)Nc3ccc(C)c(OC)c3)CC1)C(=O)N2]

### (1s,4s)-N-(3-Methoxy-4-methylphenyl)-4-(6-(methoxymethyl)-5-methyl-2-oxo-1,2-dihydroquinazolin-3(4H)-yl)cyclohexanecarboxamide

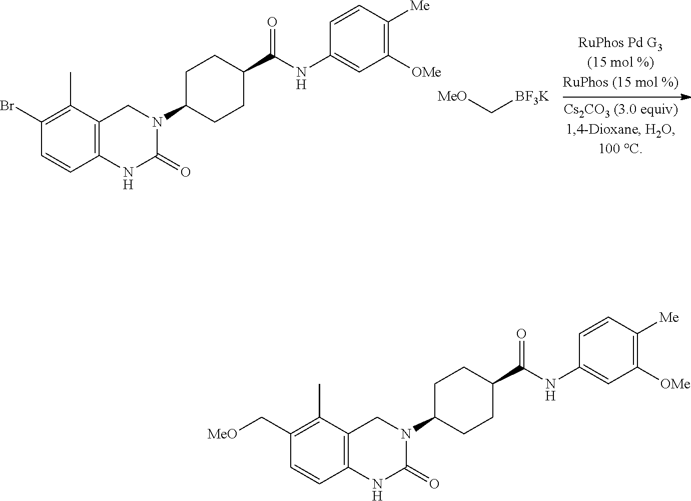

### Step 1

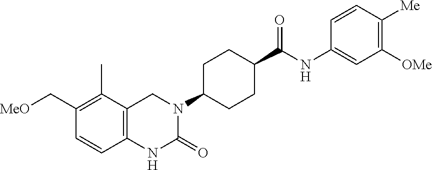 [SMILES: COCc1ccc2c(c1C)CN([C@H]1CC[C@@H](C(=O)Nc3ccc(C)c(OC)c3)CC1)C(=O)N2]

### (1s,4s)-N-(3-Methoxy-4-methylphenyl)-4-(6-(methoxymethyl)-5-methyl-2-oxo-1,2-dihydroquinazolin-3(4H)-yl)cyclohexanecarboxamide

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide from Example 63 (200 mg, 411 μmol) and it was diluted with 1,4-dioxane (2.05 mL) and Water (205 uL). The solvent was sparged with nitrogen for 5 minutes. Then, potassium trifluoro(methoxymethyl)borate (375 mg, 1.23 mmol) and RuPhos (29.4 mg, 61.7 μmol), RuPhos Pd G3 (54.3 mg, 61.7 μmol) and cesium carbonate (402 mg, 1.23 mmol) were added in that order. The vial was sealed with a microwave cap and heated to 100° C. overnight. The reaction was then cooled to room temperature and opened to air. The solvent was then evaporated to dryness and residue was purified over a gradient of 100% aqueous ammonium formate (AMF) solution (pH=3.8) to 70% MeCN in AMF was used. Product came off at 40% MeCN in AMF. Lyophilization of clean fraction provided (1s,4s)-N-(3-methoxy-4-methylphenyl)-4-(6-(methoxymethyl)-5-methyl-2-oxo-1,2-dihydroquinazolin-3(4H)-yl)cyclohexanecarboxamide as a white solid (8.50 mg, 4.2% yield). LCMS ESI m/z 452.3 [M+H]⁺. ¹H NMR (400 MHz, DMSO-d₆) δ 9.69 (s, 1H), 9.09 (s, 1H), 7.27 (s, 1H), 7.10 (d, J=8.7 Hz, 1H), 7.01 (d, J=8.1 Hz, 2H), 6.53 (d, J=7.0 Hz, 1H), 4.27 (s, 2H), 4.23-4.13 (m, 1H), 4.23 (s, 2H), 3.74 (s, 3H), 3.21 (s, 3H), 2.63-2.61 (m, 1H), 2.15-2.04 (m, 2H), 2.08 (s, 3H), 2.07 (s, 3H), 2.03-1.92 (m, 2H), 1.69-1.60 (m, 2H), 1.48-1.40 (m, 2H).

### Example 49

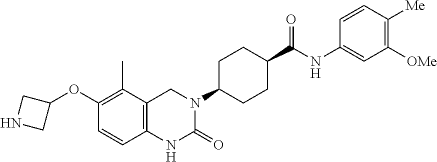 [SMILES: COc1cc(NC(=O)[C@H]2CC[C@@H](N3Cc4c(ccc(OC5CNC5)c4C)NC3=O)CC2)ccc1C]

### (1s,4s)-4-(6-(Azetidin-3-yloxy)-5-methyl-2-oxo-1,2-dihydroquinazolin-3(4H)-yl)-N-(3-methoxy-4-methylphenyl)cyclohexanecarboxamide

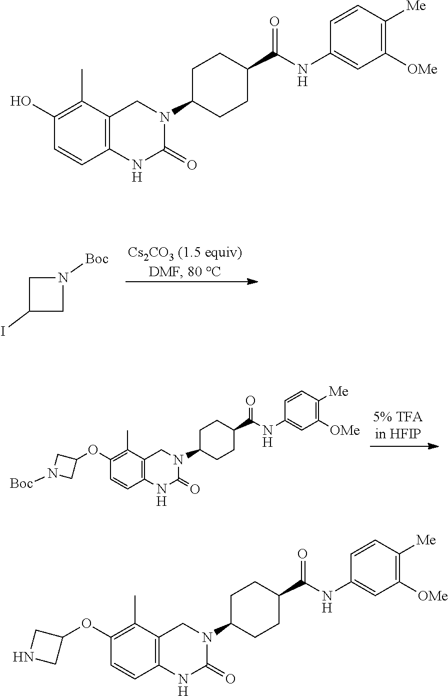 [SMILES: COc1cc(NC(=O)[C@H]2CC[C@@H](N3Cc4c(ccc(O)c4C)NC3=O)CC2)ccc1C ; CC(C)(C)OC(=O)N1CC(I)C1 ; COc1cc(NC(=O)[C@H]2CC[C@@H](N3Cc4c(ccc(OC5CN(C(=O)OC(C)(C)C)C5)c4C)NC3=O)CC2)ccc1C ; COc1cc(NC(=O)[C@H]2CC[C@@H](N3Cc4c(ccc(OC5CNC5)c4C)NC3=O)CC2)ccc1C]

### Step 1

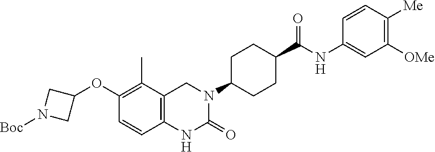 [SMILES: COc1cc(NC(=O)[C@H]2CC[C@@H](N3Cc4c(ccc(OC5CN(C(=O)OC(C)(C)C)C5)c4C)NC3=O)CC2)ccc1C]

### tert-butyl 3-((3-((1s,4s)-4-((3-Methoxy-4-methylphenyl)carbamoyl)cyclohexyl)-5-methyl-2-oxo-1,2,3,4-tetrahydroquinazolin-6-yl)oxy)azetidine-1-carboxylate

To a flame-dried, nitrogen-flushed 0.5-2.0 mL Biotage vial was added (1s,4s)-4-(6-hydroxy-5-methyl-2-oxo-1,2-dihydroquinazolin-3(4H)-yl)-N-(3-methoxy-4-methylphenyl)cyclohexanecarboxamide from Example 60 (15.0 mg, 35.4 μmol) and it was diluted with anhydrous DMF (177 μL). The solvent was sparged with nitrogen for 5 minutes. Then, cesium carbonate (17.7 mg, 53.1 μmol) and N-Boc-3-iodoazetidine (30.1 mg, 106 μmol) were added in that order. The vial was sealed with a microwave cap and heated to 80° C. over 4 hours. The reaction was then cooled to room temperature and opened to air. The crude was purified by reverse chromatography over C18. A gradient of 100% aqueous ammonium formate (AMF) solution (pH=3.8) to 70% MeCN in AMF was used. Evaporation of the fraction provided desired product as a white solid. tert-Butyl 3-((3-((1s,4s)-4-((3-Methoxy-4-methylphenyl)carbamoyl)cyclohexyl)-5-methyl-2-oxo-1,2,3,4-tetrahydroquinazolin-6-yl)oxy)azetidine-1-carboxylate

(18.0 mg, 87% yield) isolated. ¹H NMR (400 MHz, DMSO-d6) δ 9.68 (s, 1H), 8.92 (s, 1H), 7.27 (s, 1H), 7.09 (d, J=7.3 Hz, 1H), 7.00 (d, J=7.6 Hz, 1H), 6.50 (dd, J=17.5, 8.7 Hz, 2H), 4.86-4.76 (m, 1H), 4.26-4.13 (m, 4H), 3.77-3.69 (m, 4H), 2.70-2.51 (m, 3H), 2.14-1.92 (m, 4H), 2.06 (s, 3H), 2.02 (s, 3H), 1.70-1.58 (m, 2H), 1.47-1.32 (m, 2H), 1.36 (s, 9H).

### Step 2

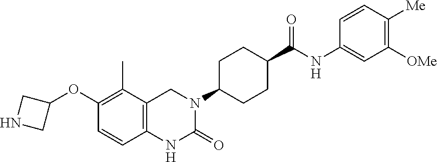 [SMILES: COc1cc(NC(=O)[C@H]2CC[C@@H](N3Cc4c(ccc(OC5CNC5)c4C)NC3=O)CC2)ccc1C]

### (1s,4s)-4-(6-(Azetidin-3-yloxy)-5-methyl-2-oxo-1,2-dihydroquinazolin-3(4H)-yl)-N-(3-methoxy-4-methylphenyl)cyclohexanecarboxamide

To a 20 mL vial which contained tert-Butyl 3-((3-((1s,4s)-4-((3-Methoxy-4-methylphenyl)carbamoyl)cyclohexyl)-5-methyl-2-oxo-1,2,3,4-tetrahydroquinazolin-6-yl)oxy)azetidine-1-carboxylate (18.0 mg, 31.1 μmol) was added a solution of 5% TFA in HFIP (2 mL) dropwise and reaction stirred for 1 hour. The crude was then evaporated to dryness to provide a translucent oil. Purification of the crude was performed using a gradient of 100% aqueous ammonium formate (AMF) solution (pH=3.8) to 70% MeCN in AMF was used. Lyophilization of the fractions provided (1s,4s)-4-(6-(Azetidin-3-yloxy)-5-methyl-2-oxo-1,2-dihydroquinazolin-3(4H)-yl)-N-(3-methoxy-4-methylphenyl)cyclohexanecarboxamide as a white solid (4.50 mg, 28% yield), as a formate salt. LCMS ESI m/z 479.4 [M+H]⁺. ¹H NMR (400 MHz, DMSO-d₆) δ 9.69 (s, 1H), 8.91 (s, 1H), 8.26 (s, 1H), 7.26 (d, J=1.3 Hz, 1H), 7.09 (dd, J=8.0, 1.5 Hz, 1H), 7.00 (d, J=8.2 Hz, 1H), 6.47 (q, J=8.7 Hz, 2H), 4.82 (p, J=6.6 Hz, 1H), 4.26-4.10 (m, 1H), 4.19 (s, 2H), 3.86 (t, J=7.6 Hz, 2H), 3.73 (s, 3H), 3.58 (dd, J=8.2, 6.7 Hz, 2H), 2.67-2.59 (m, 1H), 2.14-2.04 (m, 2H), 2.06 (s, 3H), 2.04-1.91 (m, 2H), 2.02 (s, 3H), 1.69-1.56 (m, 2H), 1.46-1.36 (m, 2H).

### Example 50

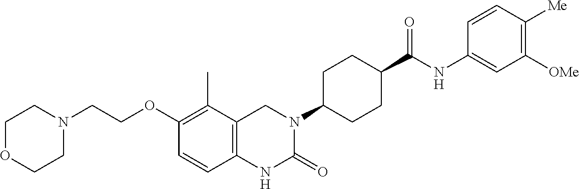 [SMILES: COc1cc(NC(=O)[C@H]2CC[C@@H](N3Cc4c(ccc(OCCN5CCOCC5)c4C)NC3=O)CC2)ccc1C]

### (1s,4s)-N-(3-Methoxy-4-methylphenyl)-4-(5-methyl-6-(2-morpholinoethoxy)-2-oxo-1,2-dihydroquinazolin-3(4H)-yl)cyclohexanecarboxamide

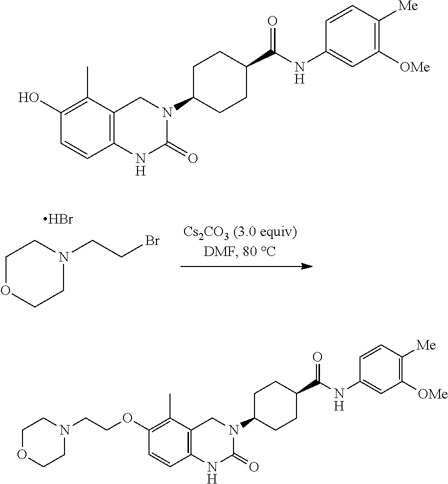 [SMILES: COc1cc(NC(=O)[C@H]2CC[C@@H](N3Cc4c(ccc(O)c4C)NC3=O)CC2)ccc1C ; Br ; BrCCN1CCOCC1 ; COc1cc(NC(=O)[C@H]2CC[C@@H](N3Cc4c(ccc(OCCN5CCOCC5)c4C)NC3=O)CC2)ccc1C]

### Step 1

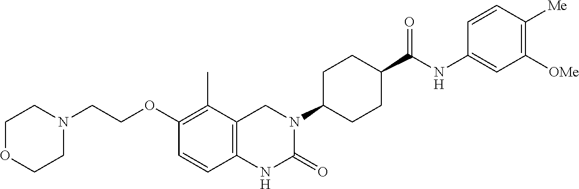 [SMILES: COc1cc(NC(=O)[C@H]2CC[C@@H](N3Cc4c(ccc(OCCN5CCOCC5)c4C)NC3=O)CC2)ccc1C]

### (1s,4s)-N-(3-Methoxy-4-methylphenyl)-4-(5-methyl-6-(2-morpholinoethoxy)-2-oxo-1,2-dihydroquinazolin-3(4H)-yl)cyclohexanecarboxamide

To a flame-dried, nitrogen-flushed 0.5-2.0 mL Biotage vial was added 1S,4S)-4-(6-hydroxy-5-methyl-2-oxo-1,2-dihydroquinazolin-3(4H)-yl)-N-(3-methoxy-4-methylphenyl)cyclohexanecarboxamide from Example 60 (15.0 mg, 35.4 μmol) and it was diluted with anhydrous DMF (177 μL). The solvent was sparged with nitrogen for 5 minutes. Then, 4-(2-bromoethyl)morpholine hydrobromide (29.2 mg, 106 μmol) and cesium carbonate (35.3 mg, 106 μmol) were added in that order. The vial was sealed with a microwave cap and heated to 80° C. in an oil bath. The reaction was stopped after 4 hours. Purification of the crude directly was performed using C18 and a gradient of 100% aqueous ammonium formate (AMF) solution (pH=3.8) to 70% MeCN in AMF was used. Fractions were evaporated to dryness over rotovap. The powder was then re-purified, this time using AMB instead of AMF. Purification of the crude was performed using 12 g C18 column. A gradient of 80% aqueous ammonium Bicarbonate (AMB) solution (pH=10.0) in 20% MeCN to 90% MeCN in AMB was used. Lyophilization of the fraction provided (1s,4s)-N-(3-Methoxy-4-methylphenyl)-4-(5-methyl-6-(2-morpholinoethoxy)-2-oxo-1,2-dihydroquinazolin-3(4H)-yl)cyclohexanecarboxamide as a white solid (1.70 mg, 8.0% yield), as a formate salt. LCMS ESI m/z 537.5 [M+H]⁺. ¹H NMR (400 MHz, DMSO-d₆+D₂O) δ 9.73 (s, 1H), 8.84 (s, 1H), 8.38 (s, 1H), 7.23 (s, 1H), 7.05-6.98 (m, 2H), 6.73 (d, J=8.8 Hz, 2H), 6.51 (d, J=8.9 Hz, 2H), 4.21 (s, 2H), 4.21-4.09 (m, 1H), 3.94 (t, J=5.7 Hz, 2H), 3.71 (s, 3H), 3.54-3.50 (m, 4H), 2.63-2.59 (m, 3H), 2.42-2.40 (m, 2H), 2.11-1.88 (m, 10H), 1.67-1.55 (m, 2H), 1.45-1.38 (m, 2H).

### Example 51

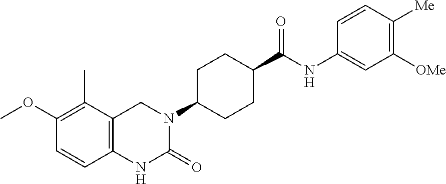 [SMILES: COc1cc(NC(=O)[C@H]2CC[C@@H](N3Cc4c(ccc(OC)c4C)NC3=O)CC2)ccc1C]

### (1s,4s)-N-(3-Methoxy-4-methylphenyl)-4-(6-methoxy-5-methyl-2-oxo-1,2-dihydroquinazolin-3(4H)-yl)cyclohexanecarboxamide

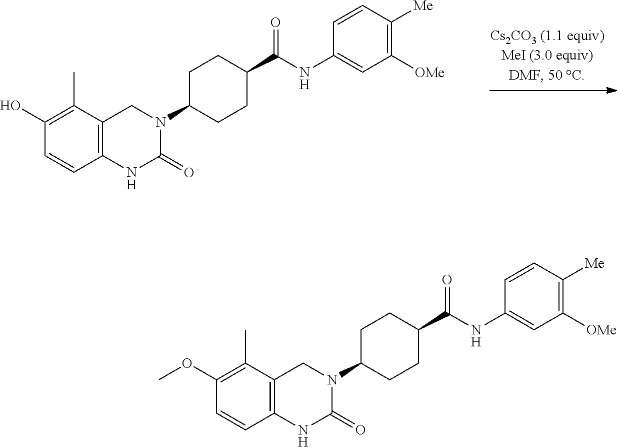 [SMILES: COc1cc(NC(=O)[C@H]2CC[C@@H](N3Cc4c(ccc(O)c4C)NC3=O)CC2)ccc1C ; COc1cc(NC(=O)[C@H]2CC[C@@H](N3Cc4c(ccc(OC)c4C)NC3=O)CC2)ccc1C]

### Step 1

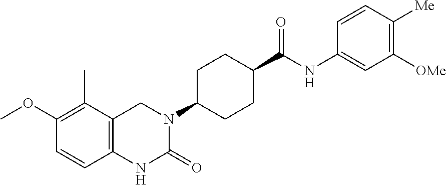 [SMILES: COc1cc(NC(=O)[C@H]2CC[C@@H](N3Cc4c(ccc(OC)c4C)NC3=O)CC2)ccc1C]

### (1s,4s)-N-(3-Methoxy-4-methylphenyl)-4-(6-methoxy-5-methyl-2-oxo-1,2-dihydroquinazolin-3(4H)-yl)cyclohexanecarboxamide

To a flame-dried, nitrogen-flushed 0.5-2.0 mL Biotage vial was added (1s,4s)-4-(6-hydroxy-5-methyl-2-oxo-1,2-dihydroquinazolin-3(4H)-yl)-N-(3-methoxy-4-methylphenyl)cyclohexanecarboxamide from Example 60 (10.0 mg, 23.6 μmol) and it was diluted with anhydrous DMF (118 μL). The solvent was sparged with nitrogen for 5 minutes. Then, iodomethane (2.97 μL, 47.2 μmol) and cesium carbonate (7.85 mg, 23.6 μmol) were added in that order. The vial was sealed with a microwave cap and heated to 50° C. and monitored by LCMS. After 1 hour, the reaction was then cooled to room temperature and opened to air. The product was then purified by reverse chromatography over 12 g C18 column. A gradient of 100% aqueous ammonium bicarbonate (AMB) solution (pH=10.0) to 80% MeCN in AMB was used. Lyophilization of the fraction provided (1s,4s)-N-(3-Methoxy-4-methylphenyl)-4-(6-methoxy-5-methyl-2-oxo-1,2-dihydroquinazolin-3(4H)-yl)cyclohexanecarboxamide as a white solid (6.50 mg, 63% yield). LCMS ESI m/z 438.3 [M+H]⁺. ¹H NMR (400 MHz, DMSO-D6) δ 9.68 (s, 1H), 8.89 (s, 1H), 7.27 (s, 1H), 7.09 (d, J=8.6 Hz, 1H), 7.00 (d, J=7.8 Hz, 1H), 6.73 (d, J=8.5 Hz, 1H), 6.53 (d, J=8.5 Hz, 1H), 4.23-4.13 (m, 2H), 3.73 (s, 3H), 3.67 (s, 3H), 2.62-2.57 (m, 1H), 2.12-2.03 (m, 2H), 2.06 (s, 3H), 2.02-1.92 (m, 2H), 1.99 (s, 3H), 1.68-1.57 (m, 2H), 1.46-1.38 (m, 2H).

### Example 52

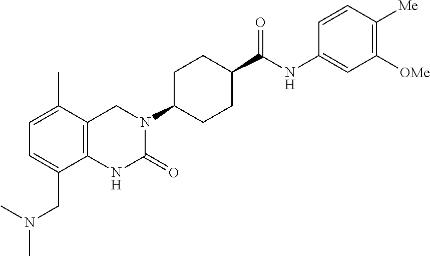 [SMILES: COc1cc(NC(=O)[C@H]2CC[C@@H](N3Cc4c(C)ccc(CN(C)C)c4NC3=O)CC2)ccc1C]

### (1s,4s)-4-(8-((Dimethylamino)methyl)-5-methyl-2-oxo-1,2-dihydroquinazolin-3(4H)-yl)-N-(3-methoxy-4-methylphenyl)cyclohexanecarboxamide

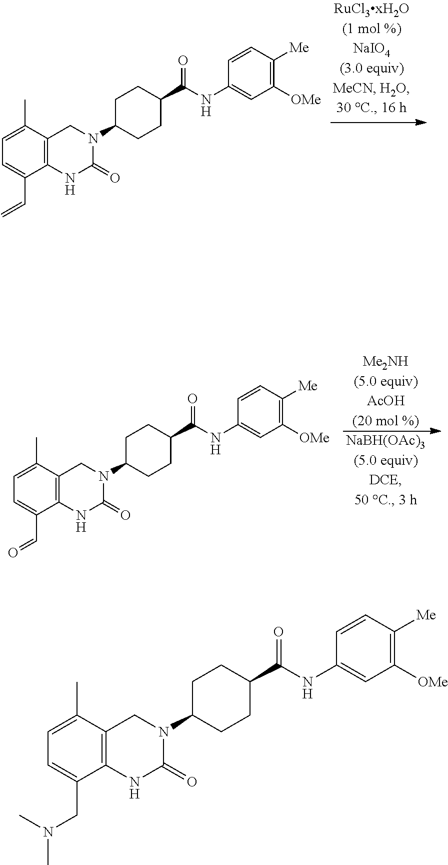 [SMILES: C=Cc1ccc(C)c2c1NC(=O)N([C@H]1CC[C@@H](C(=O)Nc3ccc(C)c(OC)c3)CC1)C2 ; COc1cc(NC(=O)[C@H]2CC[C@@H](N3Cc4c(C)ccc(C=O)c4NC3=O)CC2)ccc1C ; COc1cc(NC(=O)[C@H]2CC[C@@H](N3Cc4c(C)ccc(CN(C)C)c4NC3=O)CC2)ccc1C]

### Step 1

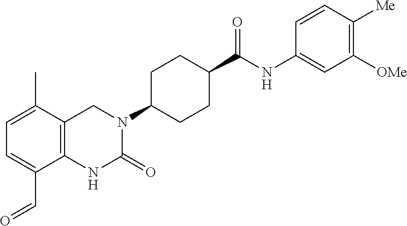 [SMILES: COc1cc(NC(=O)[C@H]2CC[C@@H](N3Cc4c(C)ccc(C=O)c4NC3=O)CC2)ccc1C]

### (1s,4s)-4-(8-Formyl-5-methyl-2-oxo-1,2-dihydroquinazolin-3(4H)-yl)-N-(3-methoxy-4-methylphenyl)cyclohexanecarboxamide

To a 20 mL scintillation vial was added (1s,4s)-N-(3-methoxy-4-methylphenyl)-4-(5-methyl-2-oxo-8-vinyl-1,2-dihydroquinazolin-3(4H)-yl)cyclohexanecarboxamide from Example 55 (30.0 mg, 69.2 μmol) and sodium periodate (44.9 mg, 208 μmol) was added along with MeCN (346 μL) and then a solution of ruthenium (III) chloride hydrate (144 ug, 0.69 μmol) in Water (346 μL). The biphasic mixture was stirred at room temperature until the next day. A second portion of ruthenium (III) chloride hydrate (144 ug, 0.69 μmol) was added and reaction stirred for an extra 3 hours. The reaction was diluted with EtOAc (20 mL) and water (10 mL). The biphasic mixture was transferred to a 60 mL extraction funnel and layers were separated. The organic layer was extracted with more EtOAc (2×20 mL), and it was washed with brine (2×20 mL), dried over Na₂SO₄, filtered and evaporated to dryness. The crude was carried to the next step without purification. (1s,4s)-4-(8-formyl-5-methyl-2-oxo-1,2-dihydroquinazolin-3(4H)-yl)-N-(3-methoxy-4-methylphenyl)cyclohexanecarboxamide was obtained (30.1 mg, 100% yield) as a green oil. LCMS ESI m/z 436.5 [M+H]⁺.

### Step 2

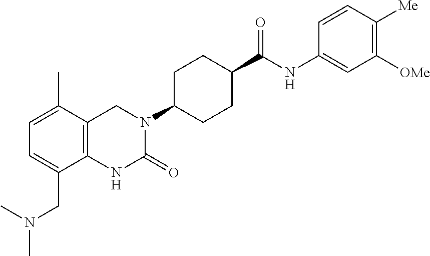 [SMILES: COc1cc(NC(=O)[C@H]2CC[C@@H](N3Cc4c(C)ccc(CN(C)C)c4NC3=O)CC2)ccc1C]

### (1s,4s)-4-(8-((Dimethylamino)methyl)-5-methyl-2-oxo-1,2-dihydroquinazolin-3(4H)-yl)-N-(3-methoxy-4-methylphenyl)cyclohexanecarboxamide

To a 20 mL scintillation vial was added (1s,4s)-4-(8-formyl-5-methyl-2-oxo-1,2-dihydroquinazolin-3(4H)-yl)-N-(3-methoxy-4-methylphenyl)cyclohexanecarboxamide (30.1 mg, 69.2 μmol) crude was added along with DCE (692 μL) and then a 5.8M solution of dimethylamine (61.8 uL, 346 μmol) in ethanol was added. Acetic acid (0.795 uL, 13.8 μmol) was added and reaction was sealed and heated to 50° C. Reaction was stirred for 30 minutes then sodium triacetoxyborohydride (75.2 mg, 346 μmol) was added. Reaction was kept stirring at 50° C. for 3 hours. The reaction was then quenched by evaporation to dryness. Direct loading on C18 and purification of the crude with a gradient of 100% aqueous ammonium bicarbonate (AMB) solution (pH=10.0) to 80% MeCN in AMB. Lyophilization of pure fractions provided (1s,4s)-4-(8-((Dimethylamino)methyl)-5-methyl-2-oxo-1,2-dihydroquinazolin-3(4H)-yl)-N-(3-methoxy-4-methylphenyl)cyclohexanecarboxamide as a white solid (3.90 mg, 12% yield). LCMS ESI m/z 465.4 [M+H]⁺. ¹H NMR (400 MHz, DMSO-d₆) δ 9.68 (s, 1H), 8.75 (s, 1H), 7.27 (d, J=1.7 Hz, 1H), 7.09 (dd, J=7.9, 1.8 Hz, 1H), 7.00 (d, J=8.2 Hz, 1H), 6.85 (d, J=7.5 Hz, 1H), 6.65 (d, J=7.7 Hz, 1H), 4.26-3.98 (m, 3H), 3.73 (s, 3H), 3.38 (s, 2H), 2.62-2.56 (m, 1H), 2.16 (s, 3H), 2.13 (s, 6H), 2.16-2.04 (m, 2H), 2.06 (s, 3H), 2.02-1.94 (m, 2H), 1.70-1.59 (m, 2H), 1.47-1.41 (m, 2H).

### Example 53

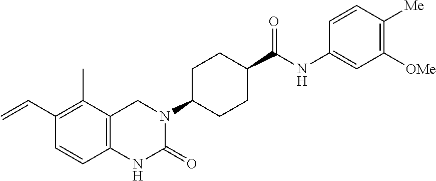 [SMILES: C=Cc1ccc2c(c1C)CN([C@H]1CC[C@@H](C(=O)Nc3ccc(C)c(OC)c3)CC1)C(=O)N2]

### (1s,4s)-N-(3-methoxy-4-methylphenyl)-4-(5-methyl-2-oxo-6-vinyl-1,2-dihydroquinazolin-3(4H)-yl)cyclohexanecarboxamide

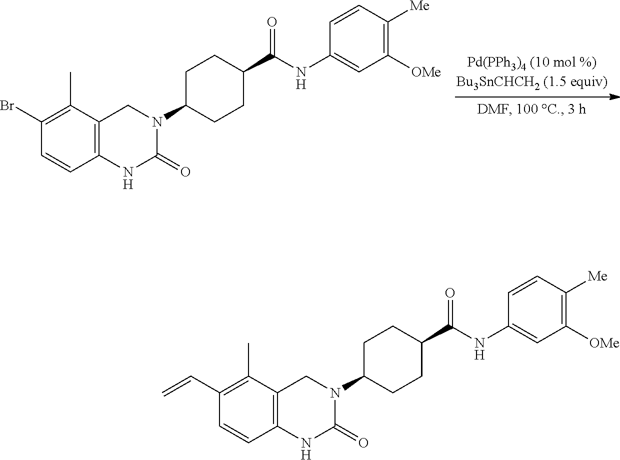 [SMILES: COc1cc(NC(=O)[C@H]2CC[C@@H](N3Cc4c(ccc(Br)c4C)NC3=O)CC2)ccc1C ; C=Cc1ccc2c(c1C)CN([C@H]1CC[C@@H](C(=O)Nc3ccc(C)c(OC)c3)CC1)C(=O)N2]

### Step 1

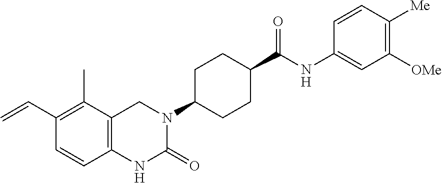 [SMILES: C=Cc1ccc2c(c1C)CN([C@H]1CC[C@@H](C(=O)Nc3ccc(C)c(OC)c3)CC1)C(=O)N2]

### (1s,4s)-N-(3-Methoxy-4-methylphenyl)-4-(5-methyl-2-oxo-6-vinyl-1,2-dihydroquinazolin-3(4H)-yl)cyclohexanecarboxamide

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide (100 mg, 206 μmol) and it was diluted with DMF (1.03 mL). The solvent was sparged with nitrogen for 5 minutes. Then, tributyl(vinyl) tin, 97% (92.9 μL, 308 μmol) and tetrakis(triphenylphosphine) palladium (0) (24.0 mg, 20.6 μmol) were added in that order. The vial was sealed with a microwave cap and heated to 100° C. for 3 hours. The reaction was then cooled to room temperature and opened to air. It resulted in a black residue which was purified by reverse phase chromatography over C18 (30 g SNAP Ultra, 100% ammonium formate solution, AMF, pH=10.0 to 70% MeCN in 30% AMF). Desired product was isolated accordingly but it was contaminated with triphenylphosphine oxide. A second purification over C18 using 30% MeCN in 70% AMF to 80% MeCN in 20% AMF was performed. Evaporation of fractions to dryness provided a white solid. (1s,4s)-N-(3-methoxy-4-methylphenyl)-4-(5-methyl-2-oxo-6-vinyl-1,2-dihydroquinazolin-3(4H)-yl)cyclohexanecarboxamide (42.0 mg, 47% yield). LCMS ESI m/z 434.4 [M+H]⁺. ¹H NMR (400 MHz, DMSO-d₆) δ 9.68 (s, 1H), 9.12 (s, 1H), 7.27-7.25 (m, 2H), 7.09 (d, J=7.7 Hz, 1H), 7.00 (d, J=7.9 Hz, 1H), 6.89 (dd, J=17.4, 11.0 Hz, 1H), 6.58 (d, J=8.2 Hz, 1H), 5.48 (d, J=16.9 Hz, 1H), 5.13 (d, J=11.1 Hz, 1H), 4.30-4.10 (m, 3H), 3.73 (s, 3H), 2.66-2.60 (m, 1H), 2.14-2.06 (m, 2H), 2.14 (s, 3H), 2.06 (s, 3H), 2.03-1.93 (m, 2H), 1.69-1.57 (m, 2H), 1.47-1.38 (m, 2H).

### Example 54

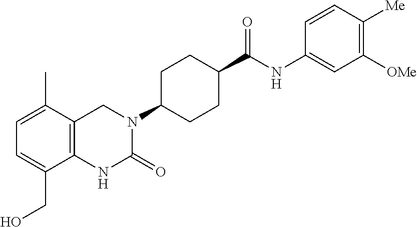 [SMILES: COc1cc(NC(=O)[C@H]2CC[C@@H](N3Cc4c(C)ccc(CO)c4NC3=O)CC2)ccc1C]

### (1s,4s)-4-(8-(Hydroxymethyl)-5-methyl-2-oxo-1,2-dihydroquinazolin-3(4H)-yl)-N-(3-methoxy-4-methylphenyl)cyclohexanecarboxamide

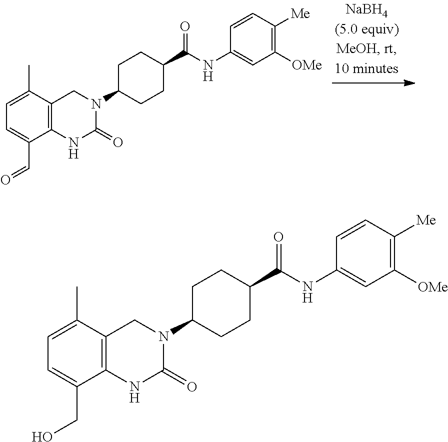 [SMILES: COc1cc(NC(=O)[C@H]2CC[C@@H](N3Cc4c(C)ccc(C=O)c4NC3=O)CC2)ccc1C ; COc1cc(NC(=O)[C@H]2CC[C@@H](N3Cc4c(C)ccc(CO)c4NC3=O)CC2)ccc1C]

### Step 1

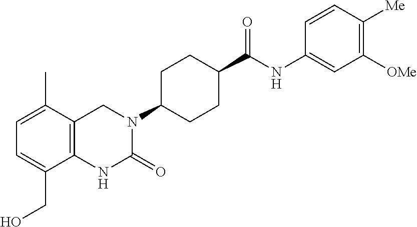 [SMILES: COc1cc(NC(=O)[C@H]2CC[C@@H](N3Cc4c(C)ccc(CO)c4NC3=O)CC2)ccc1C]

### (1s,4s)-4-(8-(Hydroxymethyl)-5-methyl-2-oxo-1,2-dihydroquinazolin-3(4H)-yl)-N-(3-methoxy-4-methylphenyl)cyclohexanecarboxamide

To a 100 mL round-bottom flask was added crude of (1s,4s)-4-(8-formyl-5-methyl-2-oxo-1,2-dihydroquinazolin-3(4H)-yl)-N-(3-methoxy-4-methylphenyl)cyclohexanecarboxamide intermediate from the preparation of Example 52 (26.9 mg, 61.7 μmol) and it was dissolved in MeOH (617 μL). The reaction was initiated by addition of sodium borohydride (11.7 μL, 308 μmol) and stirred at rt. Full conversion obtained after 5 minutes. The solvent was then evaporated to dryness and purified by reversed by C18. (30 g SNAP Ultra, 100% ammonium formate solution, AMF, pH=3.8 to 70% MeCN in 30% AMF). Evaporation of mixed fraction gave a white solid. The product was repurified twice, first over C18 and using AMB gradient (30 g SNAP Ultra, 100% ammonium bicarbonate solution, AMB, pH=10.0 to 70% MeCN in 30% AMB) followed by a purification over silica gel 50% EtOAc in Heptanes to 100% EtOAc to 10% MeOH in EtOAc provided (1s,4s)-4-(8-(Hydroxymethyl)-5-methyl-2-oxo-1,2-dihydroquinazolin-3(4H)-yl)-N-(3-methoxy-4-methylphenyl)cyclohexanecarboxamide (5.1 mg, 19% yield). LCMS ESI m/z 438.4 [M+H]⁺. ¹H NMR (400 MHz, DMSO-d₆) δ9.69 (s, 1H), 8.02 (s, 1H), 7.27 (d, J=1.5 Hz, 1H), 7.09 (dd, J=7.7, 1.7 Hz, 1H), 7.00 (d, J=8.4 Hz, 1H), 6.95 (d, J=7.8 Hz, 1H), 6.67 (d, J=7.9 Hz, 1H), 5.28 (t, J=5.4 Hz, 1H), 4.44 (d, J=5.2 Hz, 2H), 4.26-4.12 (m, 3H), 3.73 (s, 3H), 2.64-2.60 (m, 1H), 2.17-2.02 (m, 2H), 2.13 (s, 3H), 2.06 (s, 3H), 2.02-1.93 (m, 2H), 1.70-1.58 (m, 2H), 1.49-1.41 (m, 2H).

### Example 55

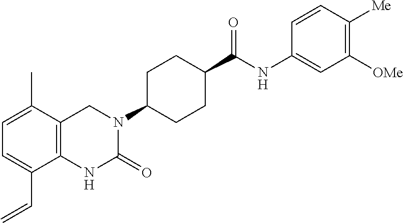 [SMILES: C=Cc1ccc(C)c2c1NC(=O)N([C@H]1CC[C@@H](C(=O)Nc3ccc(C)c(OC)c3)CC1)C2]

### (1s,4s)-N-(3-Methoxy-4-methylphenyl)-4-(5-methyl-2-oxo-8-vinyl-1,2-dihydroquinazolin-3(4H)-yl)cyclohexanecarboxamide

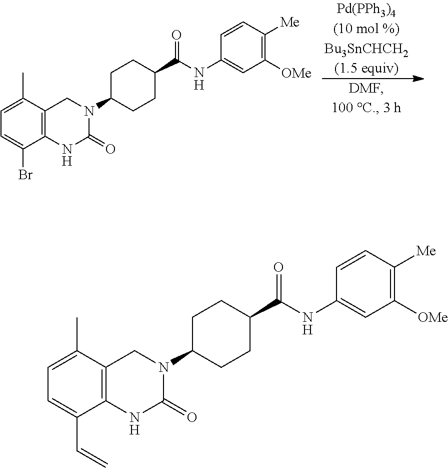 [SMILES: COc1cc(NC(=O)[C@H]2CC[C@@H](N3Cc4c(C)ccc(Br)c4NC3=O)CC2)ccc1C ; C=Cc1ccc(C)c2c1NC(=O)N([C@H]1CC[C@@H](C(=O)Nc3ccc(C)c(OC)c3)CC1)C2]

### Step 1

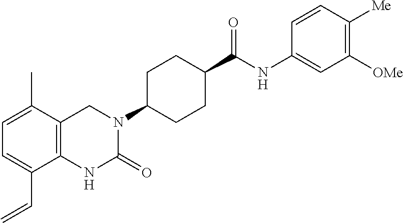 [SMILES: C=Cc1ccc(C)c2c1NC(=O)N([C@H]1CC[C@@H](C(=O)Nc3ccc(C)c(OC)c3)CC1)C2]

### (1s,4s)-N-(3-Methoxy-4-methylphenyl)-4-(5-methyl-2-oxo-8-vinyl-1,2-dihydroquinazolin-3(4H)-yl)cyclohexanecarboxamide

To a flame-dried, nitrogen-flushed 0.5-2.0 mL Biotage vial was added (1s,4s)-4-(8-bromo-5-methyl-2-oxo-1,2-dihydroquinazolin-3(4H)-yl)-N-(3-methoxy-4-methylphenyl)cyclohexanecarboxamide from Example 34 (100 mg, 206 μmol) and it was diluted with DMF (1.03 mL). The solvent was sparged with nitrogen for 5 minutes. Then, tributyl(vinyl) tin, 97% (92.9 μL, 308 μmol) and tetrakis(triphenylphosphine) palladium (0) (24.0 mg, 20.6 μmol) were added in that order. The vial was sealed with a microwave cap and heated to 100° C. for 3 hours. The reaction was then cooled to room temperature and opened to air. It resulted in a black residue which was purified by reverse phase chromatography over C18 (30 g SNAP Ultra, 100% ammonium formate solution, AMF, pH=10.0 to 70% MeCN in 30% AMF). Desired product was formed accordingly but it was contaminated with triphenylphosphine oxide. A second purification of that product following a gradient using 30% MeCN in 70% AMF to 80% MeCN in 20% AMF was performed. Evaporation of fractions to dryness provided a white solid. (1s,4s)-N-(3-Methoxy-4-methylphenyl)-4-(5-methyl-2-oxo-8-vinyl-1,2-dihydroquinazolin-3(4H)-yl)cyclohexanecarboxamide (39.0 mg, 44% yield). LCMS ESI m/z 434.2 [M+H]⁺.

¹H NMR (400 MHz, DMSO-d₆) δ 9.70 (s, 1H), 8.64 (s, 1H), 8.35 (s, 1H), 7.28-7.25 (m, 2H), 7.16-7.08 (m, 2H), 7.00 (d, J=8.5 Hz, 1H), 6.72 (d, J=8.0 Hz, 1H), 5.61 (d, J=16.9 Hz, 1H), 5.16 (d, J=12.2 Hz, 1H), 4.21-4.13 (m, 3H), 3.73 (s, 3H), 2.68-2.61 (m, 1H), 2.19-2.02 (m, 8H), 2.01-1.94 (m, 2H), 1.70-1.60 (m, 2H), 1.49-1.41 (m, 2H). Isolated as a formate salt.

### Example 56

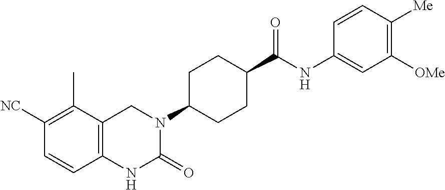 [SMILES: COc1cc(NC(=O)[C@H]2CC[C@@H](N3Cc4c(ccc(C#N)c4C)NC3=O)CC2)ccc1C]

### (1s,4s)-4-(6-Cyano-5-methyl-2-oxo-1,2-dihydroquinazolin-3(4H)-yl)-N-(3-methoxy-4-methylphenyl)cyclohexanecarboxamide

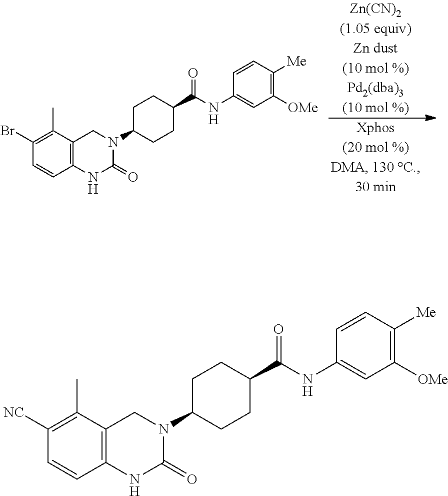 [SMILES: COc1cc(NC(=O)[C@H]2CC[C@@H](N3Cc4c(ccc(Br)c4C)NC3=O)CC2)ccc1C ; COc1cc(NC(=O)[C@H]2CC[C@@H](N3Cc4c(ccc(C#N)c4C)NC3=O)CC2)ccc1C]

### Step 1

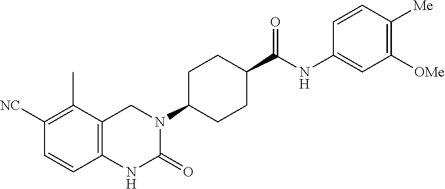 [SMILES: COc1cc(NC(=O)[C@H]2CC[C@@H](N3Cc4c(ccc(C#N)c4C)NC3=O)CC2)ccc1C]

### (1s,4s)-4-(6-Cyano-5-methyl-2-oxo-1,2-dihydroquinazolin-3(4H)-yl)-N-(3-methoxy-4-methylphenyl)cyclohexanecarboxamide

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide from Example 63 (20.0 mg, 41.1 μmol) and it was diluted with DMA (548 μL). The solvent was sparged with nitrogen for 5 minutes. Then, tris(dibenzylideneacetone)-dipalladium (0) (7.53 mg, 8.22 μmol) and zinc (0.54 mg, 8.22 μmol), zinc cyanide (9.66 mg, 82.2 μmol) and 2-dicyclohexylphosphino-2′,4′,6′-triisopropylbiphenyl (8.08 mg, 16.4 μmol) were added in that order. The vial was sealed with a microwave cap and heated to 130° C. in a microwave for 30 minutes. The reaction was then cooled to room temperature and opened to air. The solution was directly purified by reverse flash chromatography over C18 (12 g SNAP Ultra, 100% ammonium bicarbonate solution, AMB, pH=10.0 to 70% MeCN in 30% AMB). Fractions containing desired product were combined and evaporated to dryness under lyophilization. It resulted in a white solid. (1s,4s)-4-(6-Cyano-5-methyl-2-oxo-1,2-dihydroquinazolin-3(4H)-yl)-N-(3-methoxy-4-methylphenyl)cyclohexanecarboxamide (26.0 mg, 66% yield). LCMS ESI m/z 433.2 [M+H]⁺. ¹H NMR (400 MHz, DMSO-d₆) δ 9.68 (s, 1H), 9.60 (s, 1H), 7.50 (d, J=8.4 Hz, 1H), 7.27 (d, J=1.8 Hz, 1H), 7.10 (dd, J=7.9, 1.8 Hz, 1H), 7.00 (d, J=7.9 Hz, 1H), 6.69 (d, J=8.3 Hz, 1H), 4.28 (s, 2H), 4.20-4.13 (m, 1H), 3.73 (s, 3H), 2.63 (br s, 1H), 2.30 (s, 3H), 2.15-2.03 (m, 2H), 2.06 (s, 3H), 2.02-1.95 (m, 2H), 1.68-1.60 (m, 2H), 1.45-1.43 (m, 2H).

### Example 57

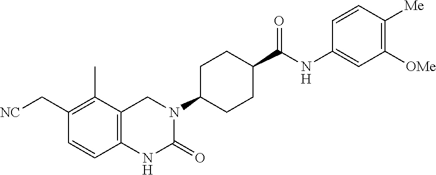 [SMILES: COc1cc(NC(=O)[C@H]2CC[C@@H](N3Cc4c(ccc(CC#N)c4C)NC3=O)CC2)ccc1C]

### (1s,4s)-4-(6-(Aminomethyl)-5-methyl-2-oxo-1,2-dihydroquinazolin-3(4H)-yl)-N-(3-methoxy-4-methylphenyl)cyclohexanecarboxamide

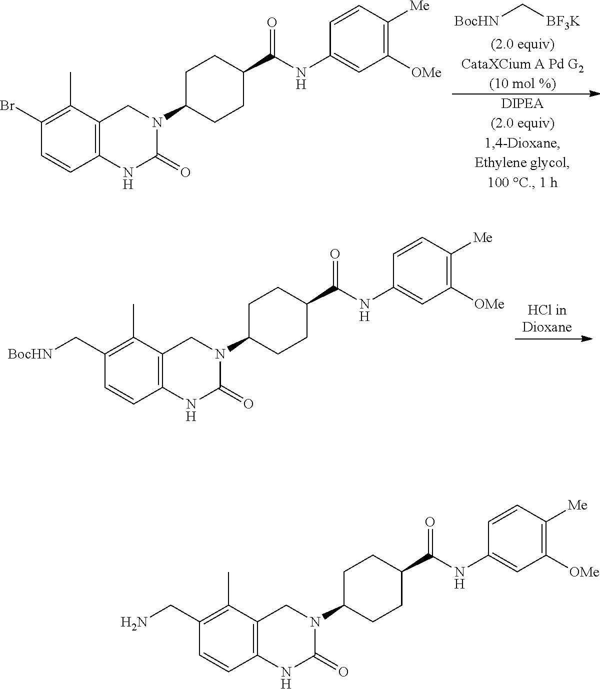

### Step 1

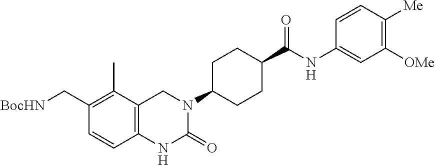 [SMILES: COc1cc(NC(=O)[C@H]2CC[C@@H](N3Cc4c(ccc(CNC(=O)OC(C)(C)C)c4C)NC3=O)CC2)ccc1C]

### tert-Butyl ((3-((1s,4s)-4-((3-methoxy-4-methylphenyl)carbamoyl)cyclohexyl)-5-methyl-2-oxo-1,2,3,4-tetrahydroquinazolin-6-yl)methyl)carbamate

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide from Example 63 (40.0 mg, 82.2 μmol) and it was diluted with 1,4-Dioxane (411 μL) and Ethylene glycol (411 μL). The solvent was sparged with nitrogen for 5 minutes. Then, chloro[(di(1-adamantyl)-N-butylphosphine)-2-(2-aminobiphenyl)]palladium (II) (CataCXium A Pd G2) (5.61 mg, 8.22 μmol) and N,N-Diisopropylethylamine (31.8 μL, 181 μmol), and potassium (((tert-butoxycarbonyl)amino)methyl)trifluoroborate (40.2 mg, 164 μmol) were added in that order. The vial was sealed with a microwave cap and heated to 100° C. for 1 hour. The reaction was then cooled to room temperature and opened to air. It resulted in a black residue which was diluted with EtOAc (30 mL) and transferred to a 60 mL extraction funnel. Brine was added and layers separated. The EtOAc layer was washed with more brine (2×30 mL), dried over Na₂SO₄, filtered and evaporated to dryness. It resulted in a black residue. The crude was carried over the next step without purification. tert-Butyl ((3-((1s,4s)-4-((3-methoxy-4-methylphenyl)carbamoyl)cyclohexyl)-5-methyl-2-oxo-1,2,3,4-tetrahydroquinazolin-6-yl)methyl)carbamate (44.1 mg, 100% yield). LCMS ESI m/z 537.2 [M+H]⁺.

### Step 2

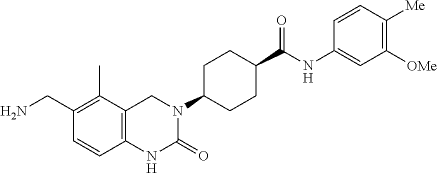 [SMILES: COc1cc(NC(=O)[C@H]2CC[C@@H](N3Cc4c(ccc(CN)c4C)NC3=O)CC2)ccc1C]

### (1s,4s)-4-(6-(aminomethyl)-5-methyl-2-oxo-1,2-dihydroquinazolin-3(4H)-yl)-N-(3-methoxy-4-methylphenyl)cyclohexanecarboxamide

To a 20 mL vial was added tert-Butyl ((3-((1s,4s)-4-((3-methoxy-4-methylphenyl)carbamoyl)cyclohexyl)-5-methyl-2-oxo-1,2,3,4-tetrahydroquinazolin-6-yl)methyl)carbamate (44.1 mg, 82.2 μmol) and it was diluted and reacted with hydrochloric acid in dioxane (20.6 μL, 82.2 μmol). Reaction was found to be done after 30 minutes at rt. Evaporation of the crude to dryness provided crude desired product. Purification of the crude was performed using C18 column and a gradient of 100% aqueous ammonium formate (AMF) solution (pH=3.8) to 70% MeCN in AMF was used. Product came off at 40% MeCN in AMF. Lyophilization of the fraction provided(1s,4s)-4-(6-(aminomethyl)-5-methyl-2-oxo-1,2-dihydroquinazolin-3(4H)-yl)-N-(3-methoxy-4-methylphenyl)cyclohexanecarboxamide as a white solid (13.0 mg, 36% yield) isolated over 2 steps. LCMS ESI m/z 537.2 [M+H]⁺. ¹H NMR (400 MHz, DMSO-d₆) δ 9.71 (s, 1H), 9.10 (s, 1H), 8.34 (s, 1H), 7.28 (s, 1H), 7.09 (t, J=7.5 Hz, 2H), 7.01 (d, J=8.0 Hz, 1H), 6.57 (d, J=7.7 Hz, 1H), 4.23 (s, 2H), 4.23-4.19 (m, 1H), 3.78-3.74 (m, 2H), 3.74 (s, 3H), 2.64 (br s, 1H), 2.12 (s, 3H), 2.07 (s, 3H), 2.12-2.04 (m, 2H), 2.04-1.92 (m, 2H), 1.70-1.57 (m, 2H), 1.48-1.38 (m, 2H). Obtained as a formate salt.

### Example 58

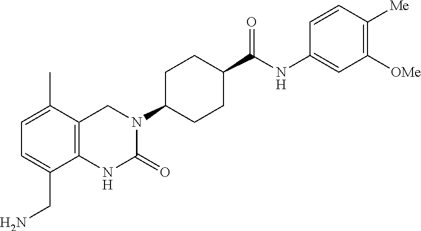 [SMILES: COc1cc(NC(=O)[C@H]2CC[C@@H](N3Cc4c(C)ccc(CN)c4NC3=O)CC2)ccc1C]

### (1s,4s)-4-(8-(Aminomethyl)-5-methyl-2-oxo-1,2-dihydroquinazolin-3(4H)-yl)-N-(3-methoxy-4-methylphenyl)cyclohexanecarboxamide

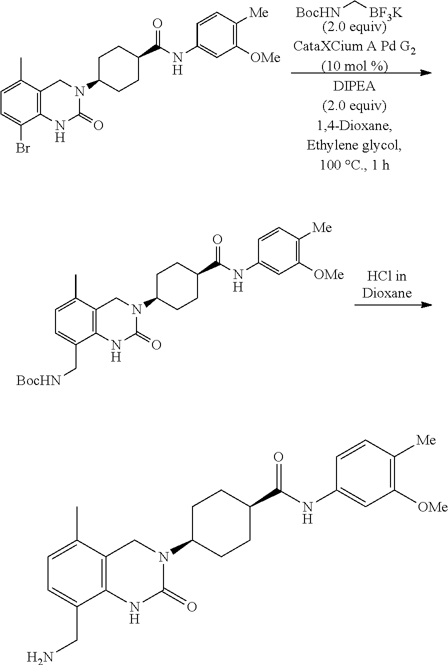

### Step 1

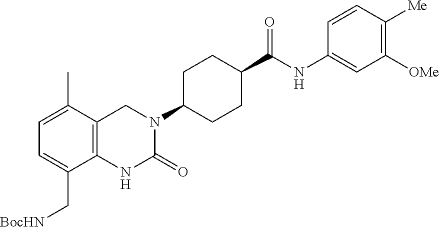 [SMILES: COc1cc(NC(=O)[C@H]2CC[C@@H](N3Cc4c(C)ccc(CNC(=O)OC(C)(C)C)c4NC3=O)CC2)ccc1C]

### tert-butyl ((3-((1s,4s)-4-((3-methoxy-4-methylphenyl)carbamoyl)cyclohexyl)-5-methyl-2-oxo-1,2,3,4-tetrahydroquinazolin-8-yl)methyl)carbamate

To a flame-dried, nitrogen-flushed 0.5-2.0 mL Biotage vial was added (1s,4s)-4-(8-bromo-5-methyl-2-oxo-1,2-dihydroquinazolin-3(4H)-yl)-N-(3-methoxy-4-methylphenyl)cyclohexanecarboxamide from Example 62 (40.0 mg, 82.2 μmol) and it was diluted with 1,4-Dioxane (411 μL) and ethylene glycol (411 μL). The solvent was sparged with nitrogen for 5 minutes. Then, chloro[(di (1-adamantyl)-N-butylphosphine)-2-(2-aminobiphenyl)]palladium (II) (5.61 mg, 8.22 μmol) and N,N-Diisopropylethylamine (31.8 μL, 181 μmol), and potassium (((tert-butoxycarbonyl)amino)methyl)trifluoroborate (40.2 mg, 164 μmol) were added in that order. The vial was sealed with a microwave cap and heated to 100° C. for 1 hour. The reaction was then cooled to room temperature and opened to air. It resulted in a black residue which was diluted with EtOAc (30 mL) and transferred to a 60 mL extraction funnel. Brine was added and layers separated. The EtOAc layer was washed with more brine (2×30 mL), dried over Na₂SO₄, filtered and evaporated to dryness. It resulted in a black residue. The crude was carried over the next step without purification. tert-butyl ((3-((1s,4s)-4-((3-methoxy-4-methylphenyl)carbamoyl)cyclohexyl)-5-methyl-2-oxo-1,2,3,4-tetrahydroquinazolin-8-yl)methyl)carbamate (44.1 mg, 100% yield) used as is and purify at next step.

### Step 2

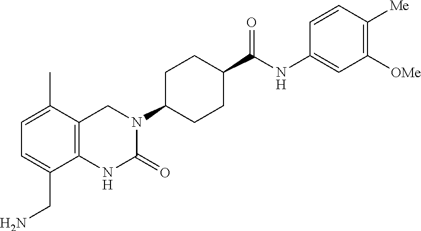 [SMILES: COc1cc(NC(=O)[C@H]2CC[C@@H](N3Cc4c(C)ccc(CN)c4NC3=O)CC2)ccc1C]

### (1s,4s)-4-(8-(Aminomethyl)-5-methyl-2-oxo-1,2-dihydroquinazolin-3(4H)-yl)-N-(3-methoxy-4-methylphenyl)cyclohexanecarboxamide

To a 20 mL vial was added tert-butyl ((3-((1s,4s)-4-((3-methoxy-4-methylphenyl)carbamoyl)cyclohexyl)-5-methyl-2-oxo-1,2,3,4-tetrahydroquinazolin-8-yl)methyl)carbamate (22.1 mg, 41.1 μmol) and it was diluted and reacted with Hydrochloric acid 4N in Dioxane (10.3 μL, 41.1 μmol). Reaction was found to be done after 30 minutes at rt. Evaporation of the crude to dryness provided desired product crude. Purification of the crude was performed using Biotage purification system over C18 column. A gradient of 100% aqueous ammonium formate (AMF) solution (pH=3.8) to 70% MeCN in AMF was used. Product came off at 40% MeCN in AMF. Lyophilization of the fraction provided (1s,4s)-4-(8-(Aminomethyl)-5-methyl-2-oxo-1,2-dihydroquinazolin-3(4H)-yl)-N-(3-methoxy-4 methylphenyl)cyclohexanecarboxamide as a white solid (5.00 mg, 25% yield) isolated over 2 steps. LCMS ESI m/z 437.2 [M+H]⁺. ¹H NMR (400 MHz, DMSO-d₆) δ 9.70 (s, 1H), 9.15 (s, 1H), 8.26 (s, 3H), 7.27 (d, J=1.6 Hz, 1H), 7.10 (dd, J=8.1, 1.7 Hz, 1H), 7.00 (d, J=8.4 Hz, 1H), 6.94 (d, J=7.7 Hz, 1H), 6.67 (d, J=7.9 Hz, 1H), 4.25-4.14 (m, 3H), 3.82 (s, 2H), 3.73 (s, 3H), 2.64 (br s, 1H), 2.17-2.08 (m, 2H), 2.14 (s, 3H), 2.02 (s, 3H), 2.02-1.94 (m, 2H), 1.71-1.60 (m, 2H), 1.47-1.41 (m, 2H). Obtained as a formate salt.

### Example 59

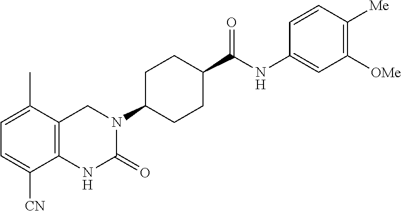 [SMILES: COc1cc(NC(=O)[C@H]2CC[C@@H](N3Cc4c(C)ccc(C#N)c4NC3=O)CC2)ccc1C]

### (1s,4s)-4-(8-Cyano-5-methyl-2-oxo-1,2-dihydroquinazolin-3(4H)-yl)-N-(3-methoxy-4-methylphenyl)cyclohexanecarboxamide

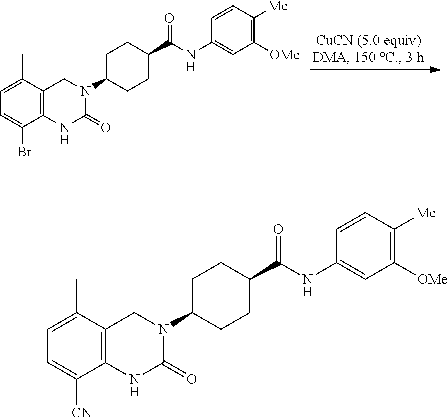 [SMILES: COc1cc(NC(=O)[C@H]2CC[C@@H](N3Cc4c(C)ccc(Br)c4NC3=O)CC2)ccc1C ; COc1cc(NC(=O)[C@H]2CC[C@@H](N3Cc4c(C)ccc(C#N)c4NC3=O)CC2)ccc1C]

### Step 1

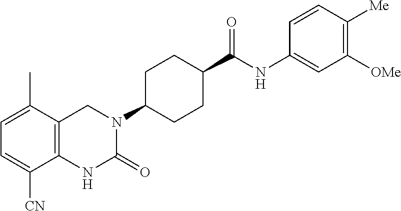 [SMILES: COc1cc(NC(=O)[C@H]2CC[C@@H](N3Cc4c(C)ccc(C#N)c4NC3=O)CC2)ccc1C]

### (1s,4s)-4-(8-Cyano-5-methyl-2-oxo-1,2-dihydroquinazolin-3(4H)-yl)-N-(3-methoxy-4-methylphenyl)cyclohexanecarboxamide

To a flame-dried, nitrogen-flushed 3 mL seal tube was added (1s,4s)-4-(8-bromo-5-methyl-2-oxo-1,2-dihydroquinazolin-3(4H)-yl)-N-(3-methoxy-4-methylphenyl)cyclohexanecarboxamide from Example 62 (30.0 mg, 61.7 μmol) along with copper (I) cyanide (27.9 mg, 308 μmol). The reagents were suspended in DMF (206 μL) anhydrous and sparged with nitrogen for 5 minutes. The tube was sealed and heated to 150° C. for 6 hours. The reaction was cooled back to room temperature and directly purified by reverse C18 purification. (30 g SNAP Ultra, 100% ammonium formate solution, AMB, pH=10.0 to 70% MeCN in 30% AMB). Product came off at 50% AMB in MeCN. Evaporation to dryness over lyophilization provided (1s,4s)-4-(8-Cyano-5-methyl-2-oxo-1,2-dihydroquinazolin-3(4H)-yl)-N-(3-methoxy-4-methylphenyl)cyclohexanecarboxamide as a yellow powder (5.40 mg, 19% yield). LCMS ESI m/z 433.2 [M+H]⁺. ¹H NMR (400 MHz, DMSO-d₆) δ 9.69 (s, 1H), 8.97 (s, 1H), 7.43 (d, J=8.0 Hz, 1H), 7.27 (d, J=1.6 Hz, 1H), 7.09 (dd, J=8.1, 1.8 Hz, 1H), 7.00 (d, J=8.1 Hz, 1H), 6.87 (d, J=8.1 Hz, 1H), 4.25 (s, 2H), 4.18-4.12 (m, 1H), 3.73 (s, 3H), 2.63 (br s, 1H), 2.22 (s, 3H), 2.13-2.05 (m, 2H), 2.01 (s, 3H), 2.01-1.95 (m, 2H), 1.68-1.61 (m, 2H), 1.46 (brd, J=9.4 Hz, 2H).

### Example 60

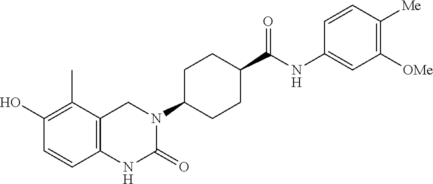 [SMILES: COc1cc(NC(=O)[C@H]2CC[C@@H](N3Cc4c(ccc(O)c4C)NC3=O)CC2)ccc1C]

### (1s,4s)-4-(6-Hydroxy-5-methyl-2-oxo-1,2-dihydroquinazolin-3(4H)-yl)-N-(3-methoxy-4-methylphenyl)cyclohexanecarboxamide

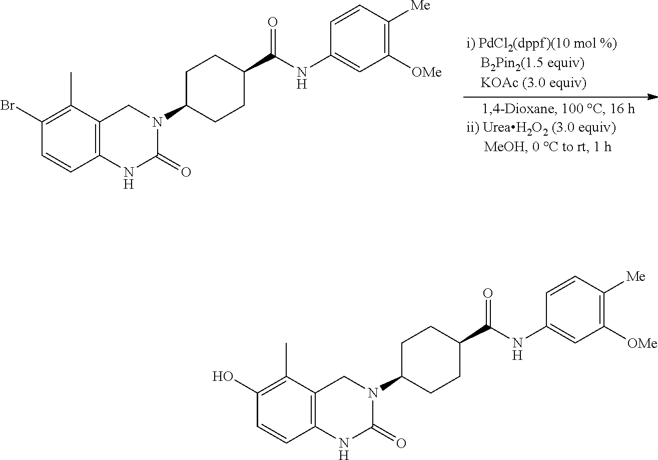 [SMILES: COc1cc(NC(=O)[C@H]2CC[C@@H](N3Cc4c(ccc(Br)c4C)NC3=O)CC2)ccc1C ; COc1cc(NC(=O)[C@H]2CC[C@@H](N3Cc4c(ccc(O)c4C)NC3=O)CC2)ccc1C]

### Step 1

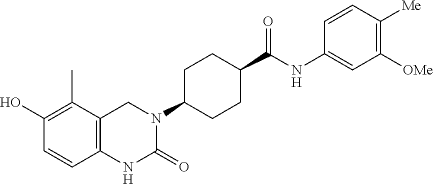 [SMILES: COc1cc(NC(=O)[C@H]2CC[C@@H](N3Cc4c(ccc(O)c4C)NC3=O)CC2)ccc1C]

### (1s,4s)-4-(6-Hydroxy-5-methyl-2-oxo-1,2-dihydroquinazolin-3(4H)-yl)-N-(3-methoxy-4-methylphenyl)cyclohexanecarboxamide

To a flame-dried, nitrogen-flushed 0.5-2.0 mL Biotage vial was added (1s,4s)-4-(6-bromo-5-methyl-2-oxo-1,2-dihydroquinazolin-3(4H)-yl)-N-(3-methoxy-4-methylphenyl)cyclohexanecarboxamide from Example 63 (20.0 mg, 41.1 μmol) and it was diluted with 1,4-Dioxane (411 μL). The solvent was sparged with nitrogen for 5 minutes. Then, 1,1′-bis(diphenylphosphino)ferrocene dichloropalladium (II) (3.07 mg, 4.11 μmol) and potassium acetate (12.4 mg, 123 μmol), and bis(pinacolato)diboron (16.1 mg, 61.7 μmol) were added in that order. The vial was sealed with a microwave cap and heated to 100° C. overnight. The reaction was then cooled to room temperature and opened to air. Methanol (1 mL) was added followed by Urea hydrogen peroxide (12.0 mg, 123 μmol) to the flask. It was found to be complete after 30 minutes. The reaction was stopped by dilution with EtOAc (20 mL) and transferred to a 60 mL extraction funnel. Water was added and layers were separated. The organic layer was separated, dried over Na₂SO₄, filtered and evaporated to dryness. It resulted in a black residue which was purified by reverse phase chromatography over C18 (12 g SNAP Ultra, 100% ammonium formate solution, AMF, pH=3.8 to 70% MeCN in 30% AMF). The fractions containing desired product were combined and lyophilized overnight. (1s,4s)-4-(6-Hydroxy-5-methyl-2-oxo-1,2-dihydroquinazolin-3(4H)-yl)-N-(3-methoxy-4-methylphenyl)cyclohexanecarboxamide (3.20 mg, 18% yield) was isolated as a beige solid. LCMS ESI m/z 424.1 [M+H]⁺. ¹H NMR (400 MHz, DMSO-d₆) δ 9.68 (s, 1H), 8.82 (s, 1H), 8.76 (s, 1H), 7.27 (d, J=1.3 Hz, 1H), 7.10 (dd, J=8.0, 1.6 Hz, 1H), 7.01 (d, J=8.1 Hz, 1H), 6.55 (d, J=8.5 Hz, 1H), 6.39 (d, J=8.4 Hz, 1H), 4.22-4.12 (m, 1H), 4.15 (s, 2H), 3.74 (s, 3H), 2.66-2.61 (m, 1H), 2.14-2.02 (m, 2H), 2.07 (s, 3H), 2.01-1.91 (m, 2H), 1.96 (s, 3H), 1.69-1.57 (m, 2H), 1.46-1.36 (m, 2H).

### Example 61

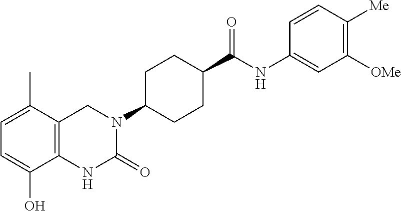 [SMILES: COc1cc(NC(=O)[C@H]2CC[C@@H](N3Cc4c(C)ccc(O)c4NC3=O)CC2)ccc1C]

### (1s,4s)-4-(8-Hydroxy-5-methyl-2-oxo-1,2-dihydroquinazolin-3(4H)-yl)-N-(3-methoxy-4-methylphenyl)cyclohexanecarboxamide

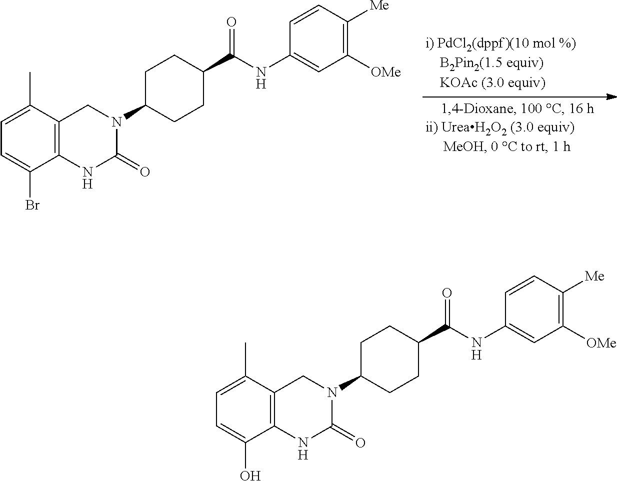 [SMILES: COc1cc(NC(=O)[C@H]2CC[C@@H](N3Cc4c(C)ccc(Br)c4NC3=O)CC2)ccc1C ; COc1cc(NC(=O)[C@H]2CC[C@@H](N3Cc4c(C)ccc(O)c4NC3=O)CC2)ccc1C]

### Step 1

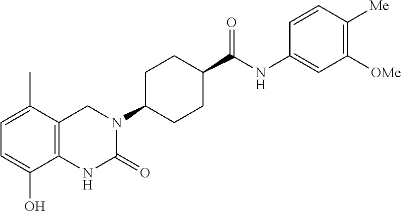 [SMILES: COc1cc(NC(=O)[C@H]2CC[C@@H](N3Cc4c(C)ccc(O)c4NC3=O)CC2)ccc1C]

### (1s,4s)-4-(8-Hydroxy-5-methyl-2-oxo-1,2-dihydroquinazolin-3(4H)-yl)-N-(3-methoxy-4-methylphenyl)cyclohexanecarboxamide

To a flame-dried, nitrogen-flushed 0.5-2.0 mL Biotage vial was added (1s,4s)-4-(8-bromo-5-methyl-2-oxo-1,2-dihydroquinazolin-3(4H)-yl)-N-(3-methoxy-4-methylphenyl)cyclohexanecarboxamide from Example 62 (30.0 mg, 61.7 μmol) and it was diluted with 1,4-dioxane (617 μL). The solvent was sparged with nitrogen for 5 minutes. Then, 1,1′-bis(diphenylphosphino)ferrocene dichloropalladium (II) (4.61 mg, 6.17 μmol) and potassium acetate (18.5 mg, 185 μmol), and bis(pinacolato)diboron (24.2 mg, 92.5 μmol) were added in that order. The vial was sealed with a microwave cap and heated to 90° C. overnight. The reaction was then cooled to room temperature and opened to air. Methanol (1 mL) was added followed by Urea hydrogen peroxide (17.9 mg, 185 μmol) to the flask. It was found to be complete after 30 minutes and the reaction was stopped by dilution with EtOAc (20 mL) and transferred to a 60 mL extraction funnel. Water was added and layers were separated. The organic layer was separated, dried over Na₂SO₄, filtered and evaporated to dryness. It resulted in a black residue which was purified by reverse phase chromatography over C18 (12 g SNAP Ultra, 100% ammonium formate solution, AMF, pH=10.0 to 70% MeCN in 30% AMF). The fractions containing desired product were combined and lyophilized overnight. (1s,4s)-4-(8-Hydroxy-5-methyl-2-oxo-1,2-dihydroquinazolin-3(4H)-yl)-N-(3-methoxy-4-methylphenyl)cyclohexanecarboxamide (6.20 mg, 23% yield) was isolated as a beige solid. LCMS ESI m/z 424.1 [M+H]⁺. ¹H NMR (400 MHz, DMSO-d₆) δ 9.69 (s, 1H), 9.36 (br s, 1H), 7.49 (s, 1H), 7.27 (s, 1H), 7.10 (dd, J=1.5, 7.5 Hz, 1H), 7.00 (d, J=8.1 Hz, 1H), 6.60-6.45 (m, 2H), 4.24-4.11 (m, 3H), 3.73 (s, 3H), 2.63 (br s, 1H), 2.06-2.00 (m, 8H), 2.02-1.89 (m, 2H), 1.67-1.61 (m, 2H), 1.45-1.42 (m, 2H).

### Example 62

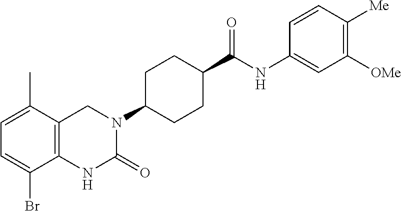 [SMILES: COc1cc(NC(=O)[C@H]2CC[C@@H](N3Cc4c(C)ccc(Br)c4NC3=O)CC2)ccc1C]

### (1s,4s)-4-(8-Bromo-5-methyl-2-oxo-1,2-dihydroquinazolin-3(4H)-yl)-N-(3-methoxy-4-methylphenyl)cyclohexanecarboxamide

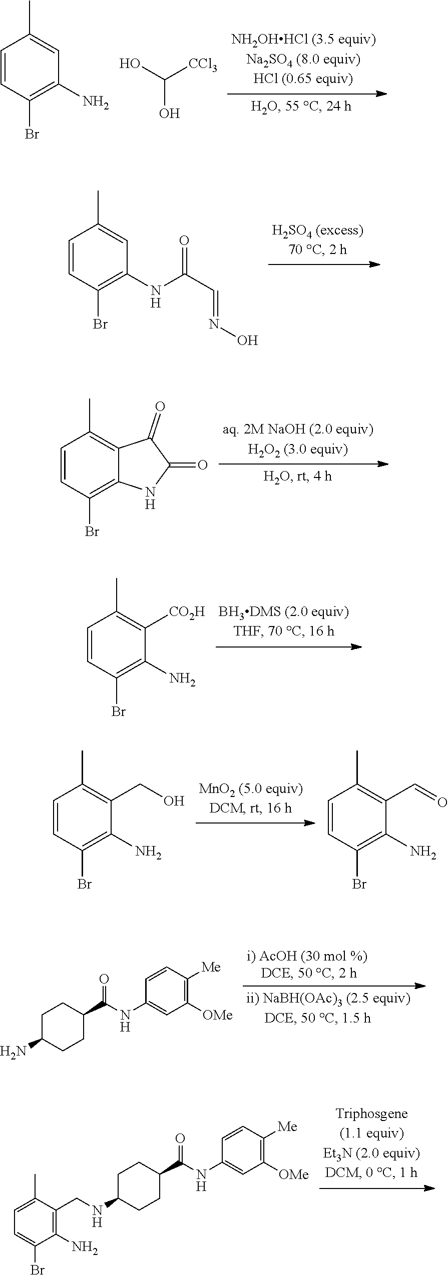

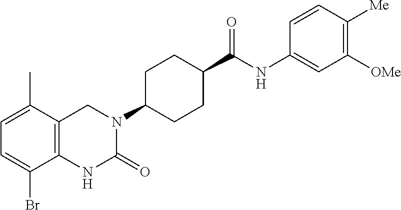 [SMILES: COc1cc(NC(=O)[C@H]2CC[C@@H](N3Cc4c(C)ccc(Br)c4NC3=O)CC2)ccc1C]

### Steps 1-7

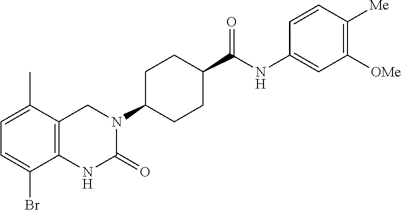 [SMILES: COc1cc(NC(=O)[C@H]2CC[C@@H](N3Cc4c(C)ccc(Br)c4NC3=O)CC2)ccc1C]

### (1s,4s)-4-(8-Bromo-5-methyl-2-oxo-1,2-dihydroquinazolin-3(4H)-yl)-N-(3-methoxy-4-methylphenyl)cyclohexanecarboxamide

The synthesis of Example 62 was already depicted in Example 34 scheme and procedures. Following these steps, (1s,4s)-4-(8-Bromo-5-methyl-2-oxo-1,2-dihydroquinazolin-3(4H)-yl)-N-(3-methoxy-4-methylphenyl)cyclohexanecarboxamide (960 mg, 78% yield) was isolated as a white solid. LCMS ESI m/z 486.0, 488.0 [M+H]⁺. ¹H NMR (400 MHz, DMSO-d₆) δ 9.69 (s, 1H), 7.74 (s, 1H), 7.29 (dd, J=11.2, 4.8 Hz, 2H), 7.09 (dd, J=8.0, 1.7 Hz, 1H), 7.00 (d, J=8.2 Hz, 1H), 6.70 (d, J=8.3 Hz, 1H), 4.25 (s, 2H), 4.17 (tt, J=12.3, 4.2 Hz, 1H), 3.73 (s, 3H), 2.67-2.60 (m, 1H), 2.16-2.04 (m, 8H), 2.02-1.93 (m, 2H), 1.70-1.59 (m, 2H), 1.48-1.42 (m, 2H).

### Example 63

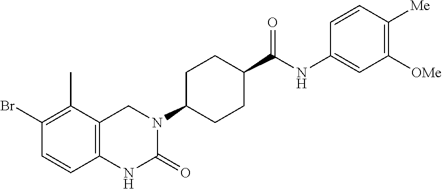 [SMILES: COc1cc(NC(=O)[C@H]2CC[C@@H](N3Cc4c(ccc(Br)c4C)NC3=O)CC2)ccc1C]

### (1s,4s)-4-(6-Bromo-5-methyl-2-oxo-1,2-dihydroquinazolin-3(4H)-yl)-N-(3-methoxy-4-methylphenyl)cyclohexanecarboxamide

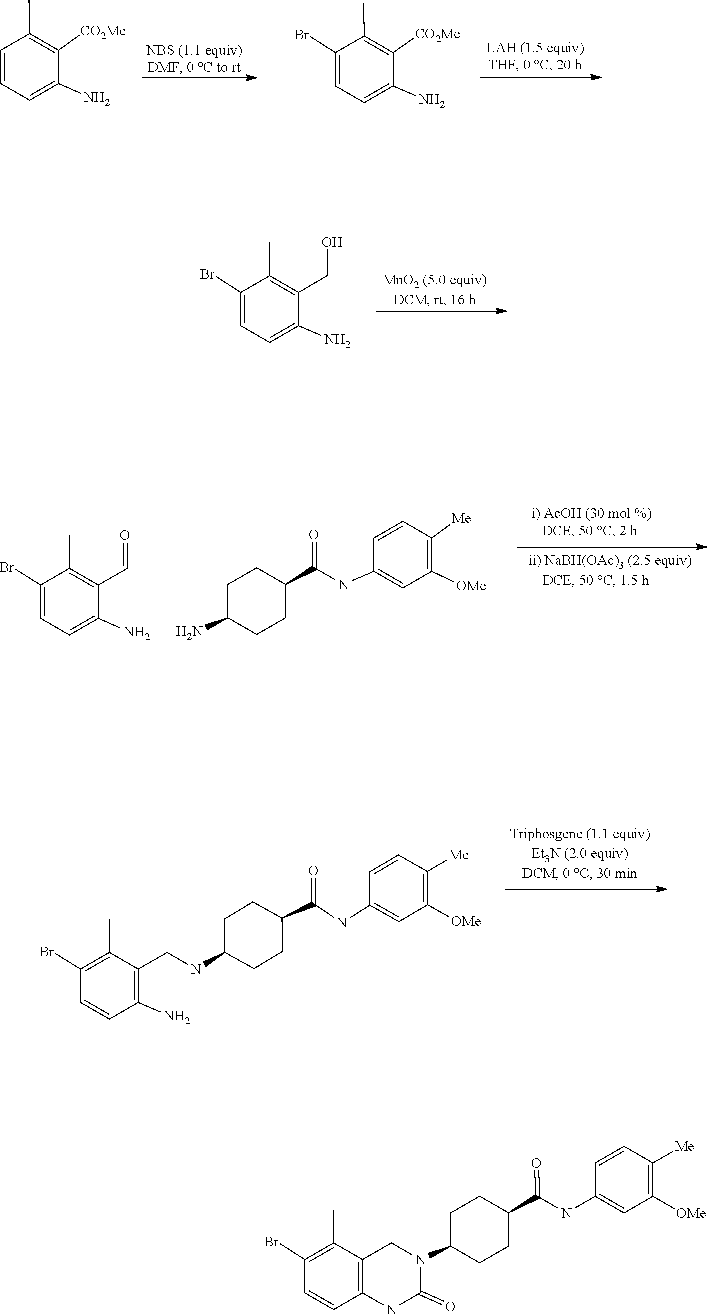

### Step 1

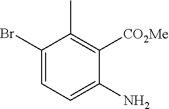

### Methyl 6-amino-3-bromo-2-methylbenzoate

To a stirred solution of methyl 2-amino-6-methylbenzoate (300 mg, 1.82 mmol) in DMF (3.63 mL) was cooled in an ice bath, and a solution of n-bromosuccinimide (323 mg, 1.82 mmol) in DMF (3.63 mL) was added dropwise during a 30 minute period, while maintaining the reaction mixture temperature at about 0-5° C. Upon completion of addition, the reaction mixture was stirred at ambient temperature for about 20 hours. After this time, the reaction mixture was poured into 12 mL of sodium bicarbonate solution. EtOAc was added and transferred to a 125 mL extraction funnel. Separate layers and aqueous layer was extracted with more EtOAc (3×20 mL). The organic extracts were combined and washed with brine (2×30 mL), dried over Na₂SO₄, filtered and evaporated to dryness. It resulted in a clear orange oil. Methyl 6-amino-3-bromo-2-methylbenzoate (412 mg, 93% yield) was isolated and carried forward without purification. LCMS ESI m/z 244.0, 246.0 [M+H]⁺. ¹H NMR (400 MHz, CDCl₃) δ 7.36 (d, J=8.7 Hz, 1H), 6.51 (d, J=8.7 Hz, 1H), 3.91 (s, 3H), 2.43 (s, 3H).

### Step 2

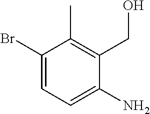

### (6-Amino-3-bromo-2-methylphenyl) methanol

To a flame-dried, nitrogen-flushed 50 mL round-bottom flask was added methyl 6-amino-3-bromo-2-methylbenzoate (12.0 g, 49.2 mmol) and it was diluted with THF (197 mL) anhydrous. The solution was cooled to 0° C., and lithium aluminum hydride (2.80 g, 73.7 mmol) solid. The solution was warmed to room temperature and stirred for 20 hours. The reaction was then quenched by addition of sodium sulfate decahydrate portionwise until gas ceased to evolve. Sodium hydroxide 2.0 M in water was added along with more sodium sulfate. The suspension was then stirred for overnight to quench the aluminum salts. After that period, suspension was filtered over Celite, cake was washed with THE wet (300 mL) and the filtrate was evaporated to dryness. (6-Amino-3-bromo-2-methylphenyl) methanol was isolated as a yellow solid (8.30 g, 78% yield). LCMS ESI m/z 216.0, 218.0 [M+H]⁺. ¹H NMR (400 MHz, DMSO-d₆) δ 7.13 (d, J=8.6 Hz, 1H), 6.45 (d, J=8.6 Hz, 1H), 5.06 (s, 2H), 4.80 (t, J=5.4 Hz, 1H), 4.43 (d, J=5.4 Hz, 2H), 2.30 (s, 3H).

### Step 3

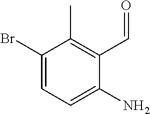

### 6-Amino-3-bromo-2-methylbenzaldehyde

To a 100 mL round-bottom flask was added (6-Amino-3-bromo-2-methylphenyl) methanol (2.00 g, 9.26 mmol) and it was diluted with DCM (46.3 mL) and the reaction was initiated by addition of manganese (IV) oxide activated (3.58 g, 37.0 mmol). The reaction was stirred at room temperature vigorously and stirred 12 hours. The reaction was stopped at that point and the black suspension was filtered over Celite. The celite cake was washed with DCM (40 mL). Filtrate was evaporated to dryness. It resulted in a yellow solid. 6-Amino-3-bromo-2-methylbenzaldehyde was isolated and carried over the next step without purification (1.90 g, 96% yield). ¹H NMR (400 MHz, CDCl₃) δ 10.40 (s, 1H), 7.41 (d, J=8.9 Hz, 1H), 6.42 (d, J=8.9 Hz, 1H), 2.66 (s, 3H).

### Step 4

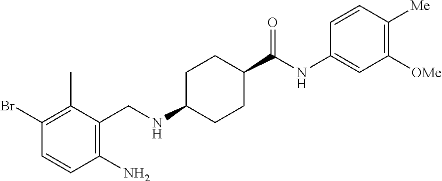 [SMILES: COc1cc(NC(=O)[C@H]2CC[C@@H](NCc3c(N)ccc(Br)c3C)CC2)ccc1C]

### (1s,4s)-4-((6-amino-3-bromo-2-methylbenzyl)amino)-N-(3-methoxy-4-methylphenyl)cyclohexanecarboxamide

To a 20 mL scintillation vial which contained 6-Amino-3-bromo-2-methylbenzaldehyde (16.0 mg, 74.7 μmol) and it was dissolved in DCE (374 μL) anhydrous. Then, (1s,4s)-4-amino-N-(3-methoxy-4-methylphenyl)cyclohexanecarboxamide (29.4 mg, 112 μmol) was added and AcOH (1 drop) was added. The solution was stirred for 30 minutes. Then, sodium triacetoxyborohydride (48.8 mg, 224 μmol) was added in one portion and the reaction was reacted at room temperature for 24 hours at rt. The reaction was stopped by evaporation of the solvent to dryness which resulted in a orange gum. The gum was purified by C18 reverse chromatography using a gradient of 100% AMF to 60% MeCN in 40% AMF. The fractions containing the desired product were combined and evaporated to dryness. (1s,4s)-4-((6-amino-3-bromo-2-methylbenzyl)amino)-N-(3-methoxy-4-methylphenyl)cyclohexanecarboxamide (19.0 mg, 55% yield) obtained as a white solid. LCMS ESI m/z 460.0, 462.0 [M+H]⁺. ¹H NMR (400 MHz, DMSO-d₆) δ 9.69 (s, 1H), 7.95 (d, J=13.6 Hz, 1H), 7.33 (s, 1H), 7.12 (d, J=8.5 Hz, 1H), 6.99 (d, J=9.0 Hz, 2H), 6.45 (d, J=8.4 Hz, 1H), 3.71 (s, 3H), 3.61 (s, 2H), 2.75 (s, 1H), 2.32 (s, 3H), 2.05 (s, 3H), 1.96-1.50 (m, 4H), 1.55-1.30 (m, 4H).

### Step 5

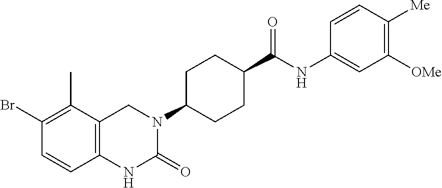 [SMILES: COc1cc(NC(=O)[C@H]2CC[C@@H](N3Cc4c(ccc(Br)c4C)NC3=O)CC2)ccc1C]

### (1s,4s)-4-(6-Bromo-5-methyl-2-oxo-1,2-dihydroquinazolin-3(4H)-yl)-N-(3-methoxy-4-methylphenyl)cyclohexanecarboxamide

To a 20 mL scintillation vial was added (1s,4s)-4-((6-amino-3-bromo-2-methylbenzyl)amino)-N-(3-methoxy-4-methylphenyl)cyclohexanecarboxamide (19.0 mg, 41.3 μmol) and it was diluted with DCM (206 μL) and cooled to 0° C. The reaction was initiated by addition of triphosgene (13.7 mg, 45.4 μmol) and triethylamine (11.6 μL, 82.5 μmol) dropwise. The reaction was found to be slightly exothermic. The reaction was found to be done after 30 minutes. The reaction was stopped by evaporation of the crude to dryness. It resulted in a white solid. Purification of the solid was done by flash chromatography using 10 g silica gel column with 50% EtOAc in Heptanes to 100% EtOAc. Evaporation to dryness provided (1s,4s)-4-(6-Bromo-5-methyl-2-oxo-1,2-dihydroquinazolin-3(4H)-yl)-N-(3-methoxy-4-methylphenyl)cyclohexanecarboxamide (7.00 mg, 35% yield) as a white solid. LCMS ESI m/z 486.1, 488.1 [M+H]⁺. ¹H NMR (400 MHz, DMSO-d₆) δ 9.68 (s, 1H), 9.21 (s, 1H), 7.32 (d, J=6.2 Hz, 1H), 7.27 (s, 1H), 7.10 (d, J=7.3 Hz, 1H), 7.01 (d, J=8.3 Hz, 1H), 6.54 (d, J=7.8 Hz, 1H), 4.27 (s, 2H), 4.17 (t, J=11.8 Hz, 1H), 3.74 (s, 3H), 2.63-2.58 (m, 1H), 2.21 (s, 3H), 2.15-1.92 (m, 7H), 1.67-1.59 (m, 2H), 1.47-1.38 (m, 2H).

### Example 64

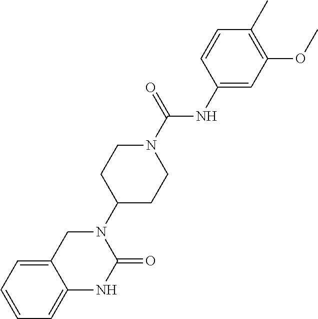 [SMILES: COc1cc(NC(=O)N2CCC(N3Cc4ccccc4NC3=O)CC2)ccc1C]

### N-(3-methoxy-4-methylphenyl)-4-(2-oxo-1,2-dihydroquinazolin-3(4H)-yl)piperidine-1-carboxamide

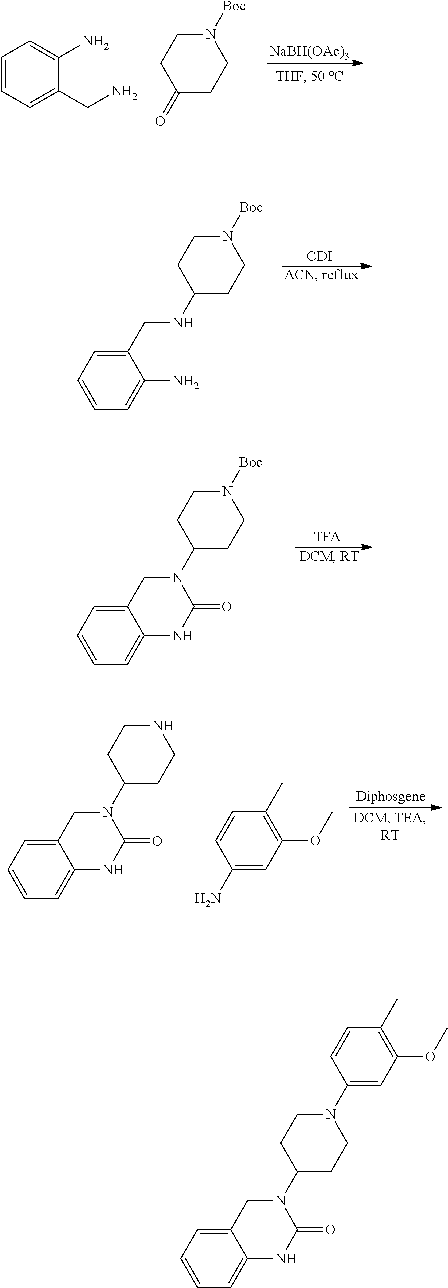

### Step 1

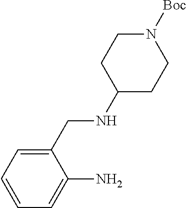 [SMILES: CC(C)(C)OC(=O)N1CCC(NCc2ccccc2N)CC1]

### tert-butyl 4-(2-aminobenzylamino) piperidine-1-carboxylate

To a solution of 2-(aminomethyl) aniline (2.44 g, 20.0 mmol) and tert-butyl 4-oxopiperidine-1-carboxylate (3.98 g, 20.0 mmol) in THF (5 mL) was added STAB (12.72 g, 60.00 mmol). The mixture was stirred at 50° C. for 3 hr. The mixture was poured into water and extracted with EtOAc. The organic extracts were washed with brine, dried by Na₂SO₄, filtered and concentrated to give a crude oil. The crude product was purified by silica gel chromatography to give tert-butyl 4-[(2-aminophenyl)methylamino]piperidine-1-carboxylate (2.0 g, 6.6 mmol, 33% yield) as a yellow solid. LCMS ESI m/z: 306.0 [M+H]⁺.

### Step 2

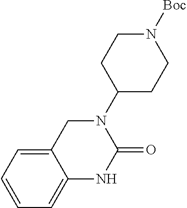 [SMILES: CC(C)(C)OC(=O)N1CCC(N2Cc3ccccc3NC2=O)CC1]

### tert-butyl 4-(2-oxo-1,2-dihydroquinazolin-3(4H)-yl)piperidine-1-carboxylate

To a solution of tert-butyl 4-[(2-aminophenyl)methylamino]piperidine-1-carboxylate (2.0 g, 6.6 mmol) in MeCN (5 mL) was added CDI (1.13 g, 7.86 mmol). The mixture was stirred at 90° C. for 3 hours. The mixture was poured into water and extracted with EtOAc. The organic extracts were washed with brine, dried by Na₂SO₄, filtered and concentrated to give a crude oil. The crude product was purified by silica gel chromatography to give tert-butyl 4-(2-oxo-1,4-dihydroquinazolin-3-yl)piperidine-1-carboxylate (2.0 g, 6.0 mmol, 92% yield) as a yellow solid. LCMS ESI m/z: 275.9 [M+H]⁺.

### Step 3

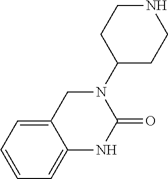 [SMILES: O=C1Nc2ccccc2CN1C1CCNCC1]

### 3-(piperidin-4-yl)-3,4-dihydroquinazolin-2(1H)-one

To a solution of tert-butyl 4-(2-oxo-1,4-dihydroquinazolin-3-yl)piperidine-1-carboxylate (2.1 g, 6.3 mmol) in DCM (28 mL) was added TFA (12 ml, 18 g, 160 mmol). The mixture was stirred at RT for 2 hours. The reaction mixture was concentrated. The residue was diluted in water, basified with NaHCO₃ until the solution reached pH=9. The solution was extracted with DCM, the combined organic phases were dried and concentrated to give 3-(piperidin-4-yl)-3,4-dihydroquinazolin-2 (1H)-one (1.3 g, 5.6 mmol, 89% yield) as a yellow oil. LCMS ESI m/z: 232.0 [M+H]⁺.

### Step 4

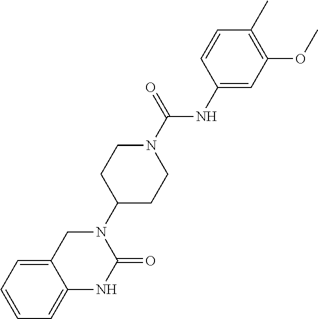 [SMILES: COc1cc(NC(=O)N2CCC(N3Cc4ccccc4NC3=O)CC2)ccc1C]

### N-(3-methoxy-4-methylphenyl)-4-(2-oxo-1,2-dihydroquinazolin-3(4H)-yl)piperidine-1-carboxamide

To a solution of diphosgene (433 mg, 2.19 mmol, 264 μL) and treithylamine (221 mg, 2.19 mmol, 305 μL) in DCM (8 mL) at 0° C. was added 3-methoxy-4-methyl-aniline (300 mg, 2.19 mmol). The solution was stirred at 0° C. for 2 hours. A solution of 3-(4-piperidyl)-1,4-dihydroquinazolin-2-one (290 mg, 1.25 mmol) and triethylamine (380 mg, 3.76 mmol, 524 μL) in DCM (10 mL) was added dropwise to the reaction mixture at 0° C. The resulting mixture was stirred at 0° C. for another 1 hour. The mixture was poured into water and extracted with DCM. The organic extracts were washed with brine, dried by Na₂SO₄, filtered and concentrated to give a crude oil. The crude product was purified by prep-HPLC to give N-(3-methoxy-4-methyl-phenyl)-4-(2-oxo-1,4-dihydroquinazolin-3-yl)piperidine-1-carboxamide (39 mg, 99 μmol, 7.9% yield) as a white solid. LCMS ESI m/z: 394.9 [M+H]⁺. ¹H NMR (400 MHz, DMSO-d₆) δ 9.21 (s, 1H), 8.43 (s, 1H), 7.18 (d, J=1.7 Hz, 1H), 7.11 (t, J=7.3 Hz, 2H), 7.02-6.96 (m, 2H), 6.88-6.82 (m, 1H), 6.77 (d, J=7.4 Hz, 1H), 4.39-4.33 (m, 1H), 4.31 (s, 2H), 4.28-4.24 (m, 2H), 3.73 (s, 3H), 2.84 (t, J=11.9 Hz, 2H), 2.06 (s, 3H), 1.79-1.68 (m, 2H), 1.61-1.56 (m, 2H).

### Example 65

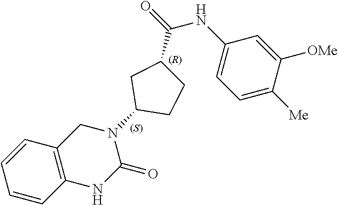

### (1R,3S)—N-(3-methoxy-4-methylphenyl)-3-(2-oxo-1,2-dihydroquinazolin-3(4H)-yl)cyclopentanecarboxamide

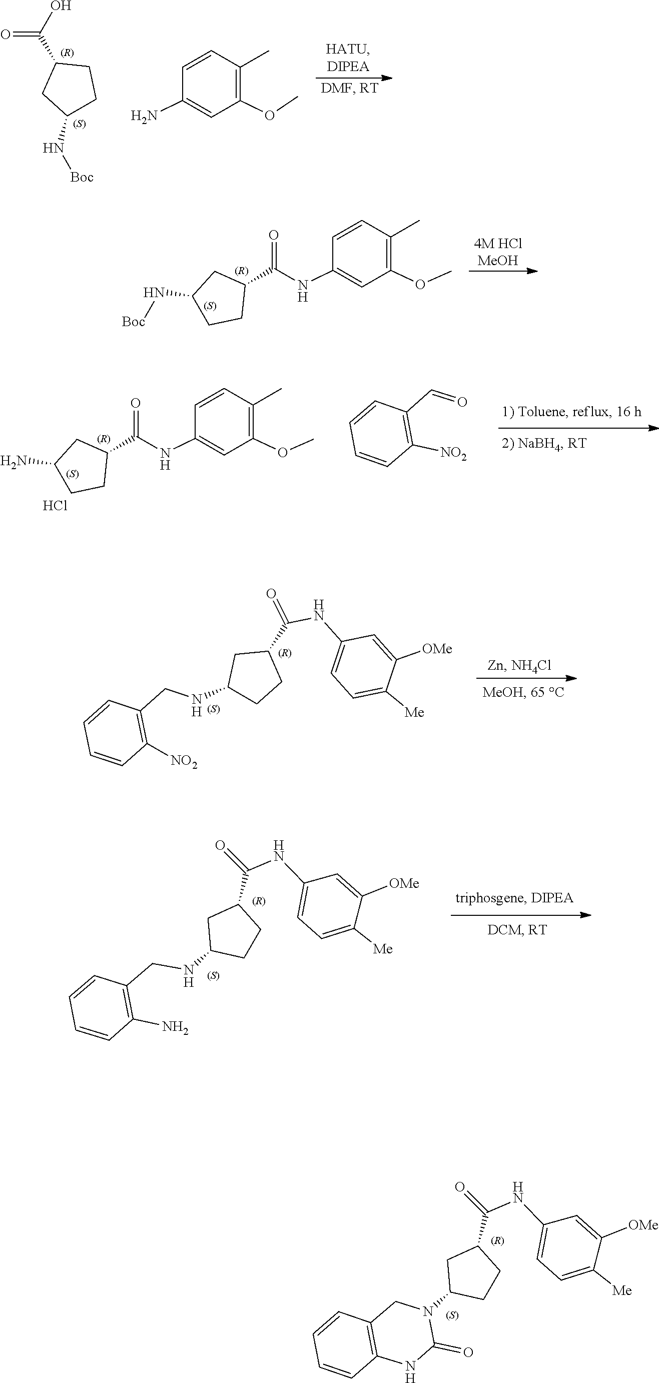

### Step 1

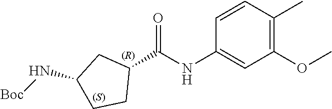

### tert-butyl (1S,3R)-3-(3-methoxy-4-methylphenylcarbamoyl)cyclopentylcarbamate

To a solution of (1R,3S)-3-(tert-butoxycarbonylamino)cyclopentanecarboxylic acid (500 mg, 2.18 mmol) in DMF (8 mL) was added HATU (1.24 g, 3.27 mmol), DIPEA (846 mg, 6.54 mmol, 1.14 mL) and 3-methoxy-4-methyl-aniline (329 mg, 2.40 mmol) at 25° C. The reaction mixture was stirred at 25° C. for 16 hours. The reaction mixture was diluted with water and extracted with EtOAc. The organic layer was dried over anhydrous Na₂SO₄, filtered and concentrated to get crude product, which was purified by silica gel chromatography to give tert-butyl (1S,3R)-3-(3-methoxy-4-methylphenylcarbamoyl)cyclopentylcarbamate (700 mg, 2.01 mmol, 92.1% yield) as a white solid. LCMS (ESI) m/z: 249.0 [M−100+H]⁺.

### Step 2

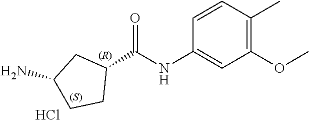

### (1R,3S)-3-amino-N-(3-methoxy-4-methylphenyl)cyclopentanecarboxamide hydrochloride

A solution of tert-butyl (1S,3R)-3-(3-methoxy-4-methylphenylcarbamoyl)cyclopentylcarbamate (700 mg, 2.01 mmol) in 4 M HCl-MeOH solution (10 mL) was stirred at 25° C. for 3 h. The mixture was concentrated to give (1R,3S)-3-amino-N-(3-methoxy-4-methylphenyl)cyclopentanecarboxamide hydrochloride (700 mg, crude) as a white solid. LCMS (ESI) m/z: 249.0 [M+H]⁺.

### Step 3

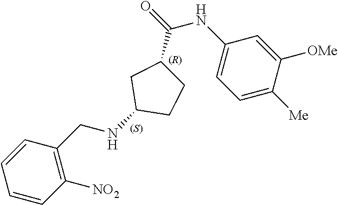

### (1R,3S)—N-(3-methoxy-4-methylphenyl)-3-(2-nitrobenzylamino)cyclopentanecarboxamide

To a solution of (1R,3S)-3-amino-N-(3-methoxy-4-methylphenyl)cyclopentanecarboxamide hydrochloride (500 mg, 2.01 mmol) in DCM (3 mL) and MeOH (1 mL) was added 2-nitrobenzaldehyde (609 mg, 4.03 mmol) at 25° C. After 30 min, NaBH(OAc)₃ (854 mg, 4.03 mmol) was added to the reaction mixture. The resulting reaction was stirred at 40° C. for 16 hours. The mixture was concentrated to get crude product, which was purified by silica gel chromatography to give (1R,3S)—N-(3-methoxy-4-methylphenyl)-3-(2-nitrobenzylamino)cyclopentanecarboxamide (300 mg, 782 μmol, 38.9% yield) as a yellow solid. LCMS (ESI) m/z: 383.9 [M+H]⁺.

### Step 4

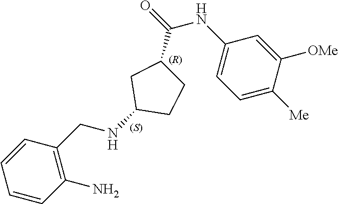

### (1R,3S)-3-(2-aminobenzylamino)-N-(3-methoxy-4-methylphenyl)cyclopentanecarboxamide

To a solution of (1R,3S)—N-(3-methoxy-4-methylphenyl)-3-(2-nitrobenzylamino)cyclopentanecarboxamide (150 mg, 391 μmol) in MeOH (2 mL) was added NH₄Cl (105 mg, 1.96 mmol) and zinc powder (128 mg, 1.96 mmol) at 25° C. The reaction was stirred at 65° C. for 2 hours. The mixture was filtered through a celite pad and the filtrate was concentrated to give (1R,3S)-3-(2-aminobenzylamino)-N-(3-methoxy-4-methylphenyl)cyclopentanecarboxamide (120 mg, crude) as a yellow solid. The crude was used directly in the next step. LCMS (ESI) m/z: 354.0 [M+H]⁺.

### Step 5

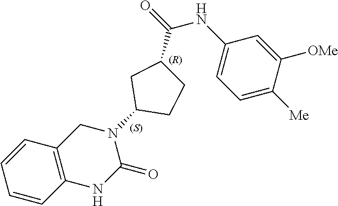

### (1R,3S)—N-(3-methoxy-4-methylphenyl)-3-(2-oxo-1,2-dihydroquinazolin-3(4H)-yl)cyclopentanecarboxamide

To a solution of (1R,3S)-3-(2-aminobenzylamino)-N-(3-methoxy-4-methylphenyl)cyclopentanecarboxamide (150 mg, 424 μmol) in DCM (2 mL) was added DIPEA (165 mg, 1.27 mmol, 222 μL) and dropwise triphosgene (139 mg, 467 μmol) in DCM (2 mL) at 0° C. The reaction was stirred at 25° C. for 1 hour. The reaction mixture was concentrated to get crude product, which was purified by prep-HPLC to give (1R,3S)—N-(3-methoxy-4-methylphenyl)-3-(2-oxo-1,2-dihydroquinazolin-3(4H)-yl)cyclopentanecarboxamide (30 mg, 79 μmol, 19% yield) as a white solid. LCMS (ESI) m/z: 379.9 [M+H]⁺. ¹H NMR (400 MHz, DMSO-d₆) δ 9.84 (s, 1H), 9.20 (s, 1H), 7.36 (s, 1H), 7.17 (d, J=7.4 Hz, 1H), 7.12 (t, J=7.6 Hz, 1H), 7.08-6.99 (m, 2H), 6.87 (t, J=7.4 Hz, 1H), 6.78 (d, J=7.9 Hz, 1H), 4.87-4.73 (m, 1H), 4.35 (s, 2H), 3.75 (s, 3H), 2.92-2.80 (m, 1H), 2.08 (s, 3H), 1.97-1.66 (m, 6H).

### Example 66

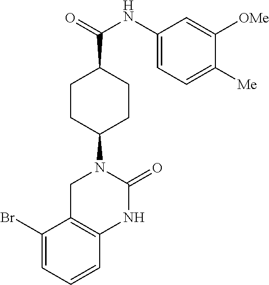 [SMILES: COc1cc(NC(=O)[C@H]2CC[C@@H](N3Cc4c(Br)cccc4NC3=O)CC2)ccc1C]

### (1s,4s)-4-(5-bromo-2-oxo-1,2-dihydroquinazolin-3(4H)-yl)-N-(3-methoxy-4-methylphenyl)cyclohexanecarboxamide

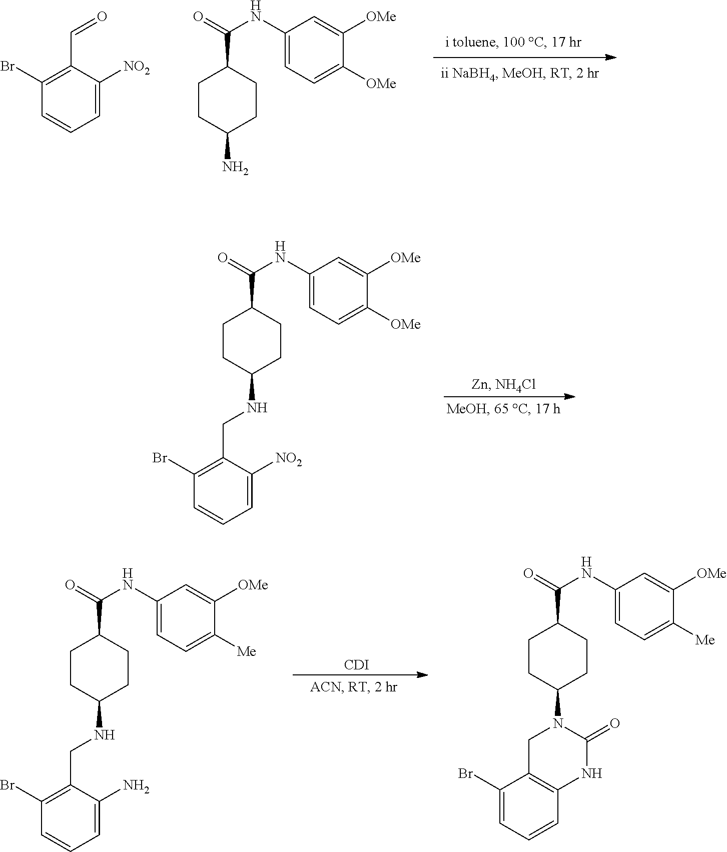 [SMILES: O=Cc1c(Br)cccc1[N+](=O)[O-] ; COc1ccc(NC(=O)[C@H]2CC[C@@H](N)CC2)cc1OC ; COc1ccc(NC(=O)[C@H]2CC[C@@H](NCc3c(Br)cccc3[N+](=O)[O-])CC2)cc1OC ; COc1cc(NC(=O)[C@H]2CC[C@@H](NCc3c(N)cccc3Br)CC2)ccc1C ; "CDI" ; COc1cc(NC(=O)[C@H]2CC[C@@H](N3Cc4c(Br)cccc4NC3=O)CC2)ccc1C]

### Step 1

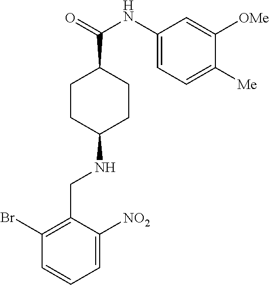 [SMILES: COc1cc(NC(=O)[C@H]2CC[C@@H](NCc3c(Br)cccc3[N+](=O)[O-])CC2)ccc1C]

### (1s,4s)-4-(2-bromo-6-nitrobenzylamino)-N-(3-methoxy-4-methylphenyl)cyclohexanecarboxamide

To a 4-amino-N-(3-methoxy-4-methyl-phenyl)cyclohexanecarboxamide (600 mg, 2.29 mmol) in toluene (20 mL) was added 2-bromo-6-nitro-benzaldehyde (500 mg, 2.17 mmol). The mixture was stirred at 100° C. for 17 hours. The reaction mixture was cooled to RT and concentrated in vacuo. The residue was diluted in MeOH (10 mL). NaBH₄ (260 mg, 6.86 mmol) was added to the mixture in portions. The resulting mixture was stirred at 25° C. for 2 hours. The mixture was poured into water (10 mL) and extracted with EtOAc (20 mL). The organic phase was dried and concentrated in vacuo. The residue was purified by silica gel chromatography (PE:EtOAc=30:1 to 1:1) to give (1s,4s)-4-(2-bromo-6-nitrobenzylamino)-N-(3-methoxy-4-methylphenyl)cyclohexanecarboxamide (500 mg, 1.05 mmol, 45.9% yield) as a yellow solid. LCMS ESI m/z 475.7 [M+H]⁺.

### Step 2

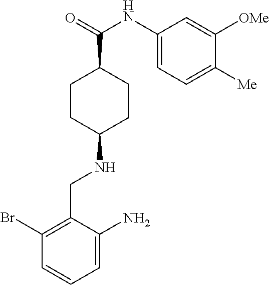 [SMILES: COc1cc(NC(=O)[C@H]2CC[C@@H](NCc3c(N)cccc3Br)CC2)ccc1C]

### (1s,4s)-4-((2-amino-6-bromobenzyl)amino)-N-(3-methoxy-4-methylphenyl)cyclohexane-1-carboxamide

To a solution of (1s,4s)-4-(2-bromo-6-nitrobenzylamino)-N-(3-methoxy-4-methylphenyl)cyclohexanecarboxamide (340 mg, 714 μmol) in MeOH (10 mL) was added NH₄Cl (191 mg, 3.57 mmol) and zinc powder (232 mg, 3.57 mmol). The mixture was stirred at 65° C. for 17 hours. The mixture was filtered and concentrated in vacuum, the residue was purified by silica gel chromatography (PE:EtOAc=30:1 to 1:1) to give (1,4s)-4-(2-amino-6-bromobenzylamino)-N-(3-methoxy-4-methylphenyl)cyclohexanecarboxamide (300 mg, 672 μmol, 94.2% yield) as a yellow solid. LCMS ESI m/z 447.8 [M+H]⁺.

### Step 3

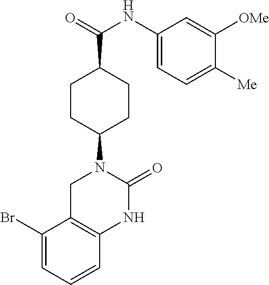 [SMILES: COc1cc(NC(=O)[C@H]2CC[C@@H](N3Cc4c(Br)cccc4NC3=O)CC2)ccc1C]

### (1s,4s)-4-(5-bromo-2-oxo-1,2-dihydroquinazolin-3(4H)-yl)-N-(3-methoxy-4-methylphenyl)cyclohexanecarboxamide

A mixture of (1s,4s)-4-(2-amino-6-bromobenzylamino)-N-(3-methoxy-4-methylphenyl)cyclohexanecarboxamide (340 mg, 762 μmol) and CDI (370 mg, 2.29 mmol) in MeCN (10 mL) was stirred at 25° C. for 2 hours. The reaction mixture was concentrated and purified by silica gel chromatography (PE:EtOAc=30:1-1:1) to give (1s,4s)-4-(5-bromo-2-oxo-1,2-dihydroquinazolin-3(4H)-yl)-N-(3-methoxy-4-methylphenyl)cyclohexanecarboxamide (300 mg, 635 μmol, 83.4% yield) as a white solid. LCMS ESI m/z 474.8 [M+H]⁺. ¹H NMR (400 MHz, DMSO-d₆) δ 9.69 (s, 1H), 9.40 (s, 1H), 7.28 (d, J=1.6 Hz, 1H), 7.17-6.92 (m, 4H), 6.76 (dd, J=7.5, 1.4 Hz, 1H), 4.30 (s, 2H), 4.22-4.17 (m, 1H), 3.75 (s, 3H), 2.66-2.64 (m, 1H), 2.19-1.89 (m, 7H), 1.73-1.56 (m, 2H), 1.49-1.45 (m, 2H).

### Example 67

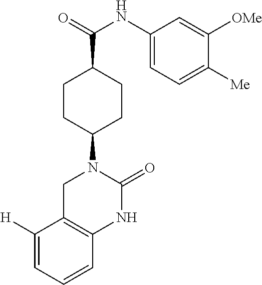 [SMILES: COc1cc(NC(=O)[C@H]2CC[C@@H](N3Cc4ccccc4NC3=O)CC2)ccc1C]

### (1s,4s)-N-(3-methoxy-4-methylphenyl)-4-(2-oxo-1,2-dihydroquinazolin-3(4H)-yl)cyclohexanecarboxamide

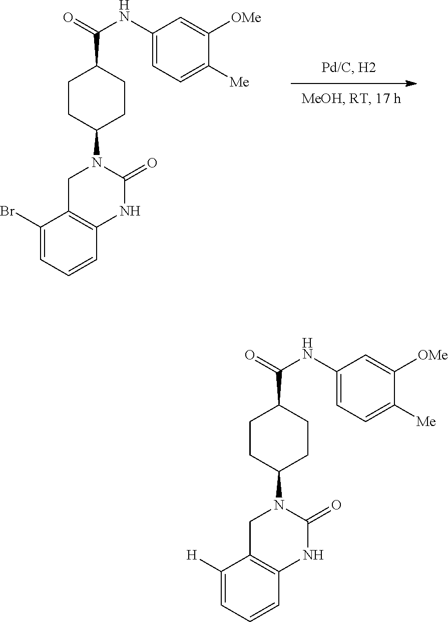 [SMILES: COc1cc(NC(=O)[C@H]2CC[C@@H](N3Cc4c(Br)cccc4NC3=O)CC2)ccc1C ; COc1cc(NC(=O)[C@H]2CC[C@@H](N3Cc4ccccc4NC3=O)CC2)ccc1C]

### Step 1

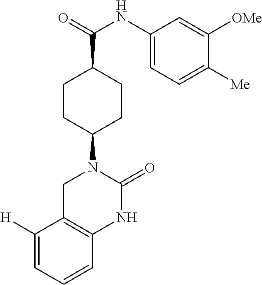 [SMILES: COc1cc(NC(=O)[C@H]2CC[C@@H](N3Cc4ccccc4NC3=O)CC2)ccc1C]

### (1s,4s)-N-(3-methoxy-4-methylphenyl)-4-(2-oxo-1,2-dihydroquinazolin-3(4H)-yl)cyclohexanecarboxamide

To a solution of (1s,4s)-4-(5-bromo-2-oxo-1,2-dihydroquinazolin-3(4H)-yl)-N-(3-methoxy-4-methylphenyl)cyclohexanecarboxamide (30 mg, 64 μmol) in MeOH (5 mL) was added Pd/C (64 μmol). The mixture was stirred at 25° C. for 17 hours under hydrogen atmosphere (balloon). The mixture was filtered and concentrated. The residue was purified by prep-HPLC to give (1s,4s)-N-(3-methoxy-4-methylphenyl)-4-(2-oxo-1,2-dihydroquinazolin-3(4H)-yl)cyclohexanecarboxamide (5.5 mg, 14 μmol, 22% yield) as a white solid. LCMS ESI m/z 393.9 [M+H]⁺. ¹H NMR (400 MHz, DMSO-d₆) δ 9.68 (s, 1H), 9.14 (s, 1H), 7.30 (d, J=1.4 Hz, 1H), 7.16-7.01 (m, 4H), 6.89-6.67 (m, 2H), 4.25 (s, 2H), 4.22-4.18 (m, 1H), 3.76 (s, 3H), 2.66-2.64 (m, 1H), 2.21-1.89 (m, 7H), 1.75-1.57 (m, 2H), 1.46-1.41 (m, 2H).

### Example 68

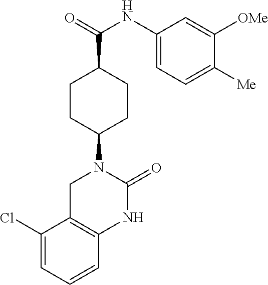 [SMILES: COc1cc(NC(=O)[C@H]2CC[C@@H](N3Cc4c(Cl)cccc4NC3=O)CC2)ccc1C]

### (1s,4s)-4-(5-chloro-2-oxo-1,2-dihydroquinazolin-3(4H)-yl)-N-(3-methoxy-4-methylphenyl)cyclohexanecarboxamide

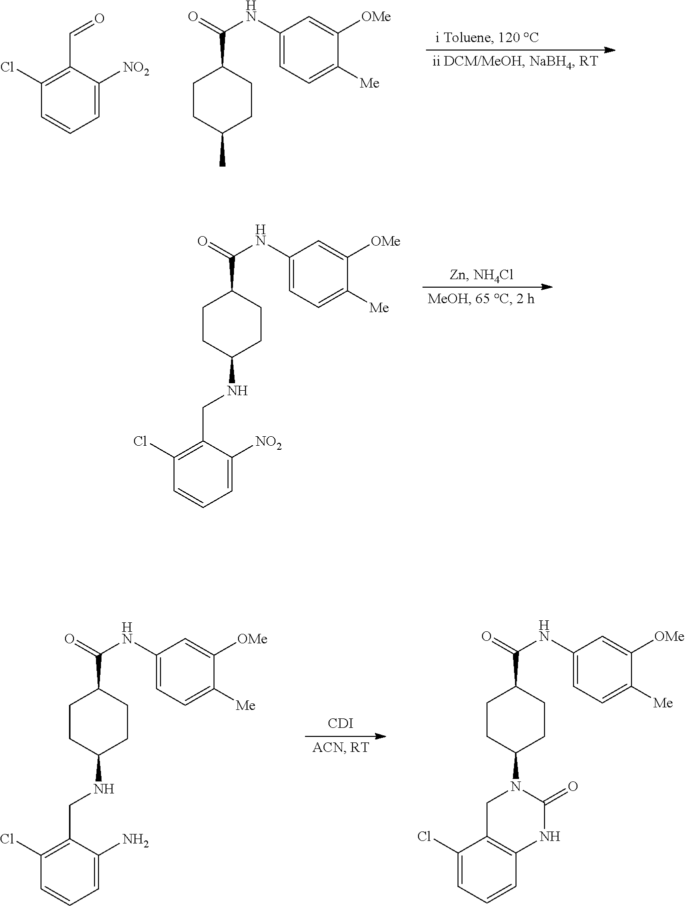 [SMILES: O=Cc1c(Cl)cccc1[N+](=O)[O-] ; COc1cc(NC(=O)[C@H]2CC[C@@H](C)CC2)ccc1C ; COc1cc(NC(=O)[C@H]2CC[C@@H](NCc3c(Cl)cccc3[N+](=O)[O-])CC2)ccc1C ; COc1cc(NC(=O)[C@H]2CC[C@@H](NCc3c(N)cccc3Cl)CC2)ccc1C ; "CDI" ; COc1cc(NC(=O)[C@H]2CC[C@@H](N3Cc4c(Cl)cccc4NC3=O)CC2)ccc1C]

### Step 1

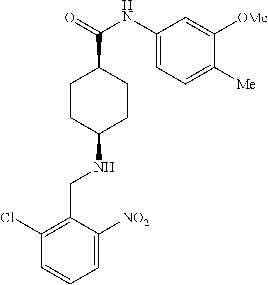 [SMILES: COc1cc(NC(=O)[C@H]2CC[C@@H](NCc3c(Cl)cccc3[N+](=O)[O-])CC2)ccc1C]

### (1s,4s)-4-(2-chloro-6-nitrobenzylamino)-N-(3-methoxy-4-methylphenyl)cyclohexanecarboxamide

A mixture of 2-chloro-6-nitro-benzaldehyde (200 mg, 1.08 mmol) and 4-amino-N-(3-methoxy-4-methyl-phenyl)cyclohexanecarboxamide (283 mg, 1.08 mmol) in toluene (10 mL) was stirred at 120° C. for 4 hours. The mixture was concentrated under reduced pressure to afford the crude product. The residue was diluted in MeOH/DCM=2/1 (10 mL). NaBH₄ (122 mg, 3.23 mmol) was added to the reaction mixture. The resulting mixture was stirred at rt for 3 hours. The mixture was diluted with water (20 mL) and extracted with EtOAc (30 mL*3). The organic layers were dried over anhydrous Na₂SO₄, filtered and concentrated to get crude product, which was purified by silica gel chromatography give to (1s,4s)-4-(2-chloro-6-nitrobenzylamino)-N-(3-methoxy-4-methylphenyl)cyclohexanecarboxamide (190 mg, 40% yield). LCMS (ESI) m/z: 431.8 [M+H]⁺.

### Step 2

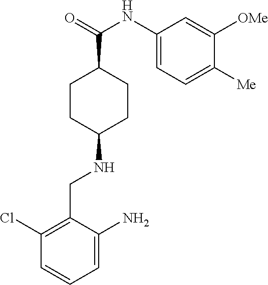 [SMILES: COc1cc(NC(=O)[C@H]2CC[C@@H](NCc3c(N)cccc3Cl)CC2)ccc1C]

### (1s,4s)-4-(2-amino-6-chlorobenzylamino)-N-(3-methoxy-4-methylphenyl)cyclohexanecarboxamide

A mixture of (1s,4s)-4-(2-chloro-6-nitrobenzylamino)-N-(3-methoxy-4-methylphenyl)cyclohexanecarboxamide (188 mg, 435 μmol), zinc powder (142 mg, 2.18 mmol) and NH₄Cl (228 mg, 4.35 mmol) in MeOH (5 mL) was stirred at 65° C. for 2 hours. The mixture was filtered and concentrated to get crude product, which was purified by silica gel chromatography give (1s,4s)-4-(2-amino-6-chlorobenzylamino)-N-(3-methoxy-4-methylphenyl)cyclohexanecarboxamide (150 mg, 85% yield). LCMS (ESI) m/z: 431.8 [M+H]⁺.

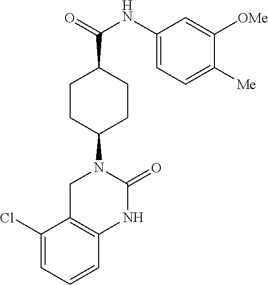 [SMILES: COc1cc(NC(=O)[C@H]2CC[C@@H](N3Cc4c(Cl)cccc4NC3=O)CC2)ccc1C]

### (1s,4s)-4-(5-chloro-2-oxo-1,2-dihydroquinazolin-3(4H)-yl)-N-(3-methoxy-4-methylphenyl)cyclohexanecarboxamide

A solution of (1s,4s)-4-(2-amino-6-chlorobenzylamino)-N-(3-methoxy-4-methylphenyl)cyclohexanecarboxamide (150 mg, 373 μmol) and di(imidazol-1-yl)methanone (181 mg, 1.12 mmol) in ACN (5 mL) was stirred at rt for 2 hours under nitrogen. The mixture was concentrated to get crude product, which was purified by prep-HPLC to give (1s,4s)-4-(5-chloro-2-oxo-1,2-dihydroquinazolin-3(4H)-yl)-N-(3-methoxy-4-methylphenyl)cyclohexanecarboxamide (11 mg, 5.0% yield) as a white solid. LCMS (ESI) m/z: 412.8 [M+H]⁺. ¹H NMR (400 MHz, DMSO-d₆) δ 9.69 (s, 1H), 9.41 (s, 1H), 7.28 (d, J=1.7 Hz, 1H), 7.17-7.08 (m, 2H), 7.02 (d, J=8.5 Hz, 1H), 6.95 (dd, J=8.0, 0.9 Hz, 1H), 6.78-6.69 (m, 1H), 4.33 (s, 2H), 4.22-4.18 (m, 1H), 3.75 (s, 3H), 2.66-2.64 (m, 1H), 2.14-2.10 (m, 2H), 2.08 (s, 3H), 2.02-1.96 (m, 2H), 1.68-1.64 (m, 2H), 1.49-1.46 (m, 2H).

### Example 69

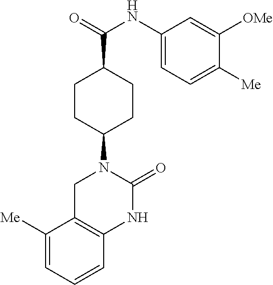 [SMILES: COc1cc(NC(=O)[C@H]2CC[C@@H](N3Cc4c(C)cccc4NC3=O)CC2)ccc1C]

### (1s,4s)-N-(3-methoxy-4-methylphenyl)-4-(5-methyl-2-oxo-1,2-dihydroquinazolin-3(4H)-yl)cyclohexanecarboxamide

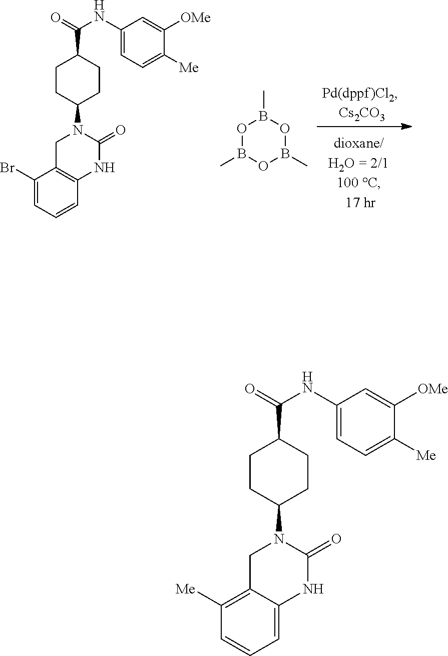

### Step 1

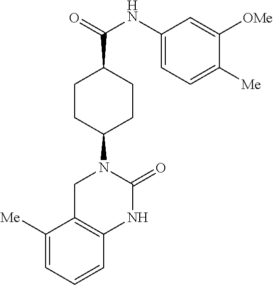 [SMILES: COc1cc(NC(=O)[C@H]2CC[C@@H](N3Cc4c(C)cccc4NC3=O)CC2)ccc1C]

### (1s,4s)-N-(3-methoxy-4-methylphenyl)-4-(5-methyl-2-oxo-1,2-dihydroquinazolin-3(4H)-yl)cyclohexanecarboxamide

To a solution of 2,4,6-trimethyl-1,3,5,2,4,6-trioxatriborinane (3.5 M, 182 μL) and 4-(5-bromo-2-oxo-1,4-dihydroquinazolin-3-yl)-N-(3-methoxy-4-methyl-phenyl)cyclohexanecarboxamide (151 mg, 319 μmol) in H₂O (1 mL) and dioxane (2 mL) was added Cs₂CO₃ (311 mg, 956 μmol) and Pd(dppf)Cl₂ (10 mg, 32 μmol). The mixture was stirred at 100° C. for 17 hours under nitrogen. The mixture was filtered and concentrated. The residue was purified by prep-HPLC to give (1s,4s)-N-(3-methoxy-4-methylphenyl)-4-(5-methyl-2-oxo-1,2-dihydroquinazolin-3(4H)-yl)cyclohexanecarboxamide (10 mg, 25 μmol, 7.7% yield) as a white solid. LCMS ESI m/z 407.9 [M+H]⁺, ¹HNMR (400 MHz, DMSO-d₆) δ 9.68 (s, 1H), 9.05 (s, 1H), 7.29 (d, J=1.7 Hz, 1H), 7.12 (dd, J=8.0, 1.8 Hz, 1H), 7.05-6.95 (m, 2H), 6.68 (d, J=7.5 Hz, 1H), 6.58 (d, J=7.8 Hz, 1H), 4.24-4.20 (m, 3H), 3.75 (s, 3H), 2.66-2.64 (m, 1H), 2.19-1.93 (m, 10H), 1.72-1.59 (m, 2H), 1.48-1.44 (m, 2H).

### Example 70

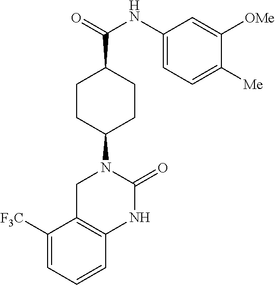 [SMILES: COc1cc(NC(=O)[C@H]2CC[C@@H](N3Cc4c(cccc4C(F)(F)F)NC3=O)CC2)ccc1C]

### (1s,4s)-N-(3-methoxy-4-methylphenyl)-4-(2-oxo-5-(trifluoromethyl)-1,2-dihydroquinazolin-3(4H)-yl)cyclohexanecarboxamide

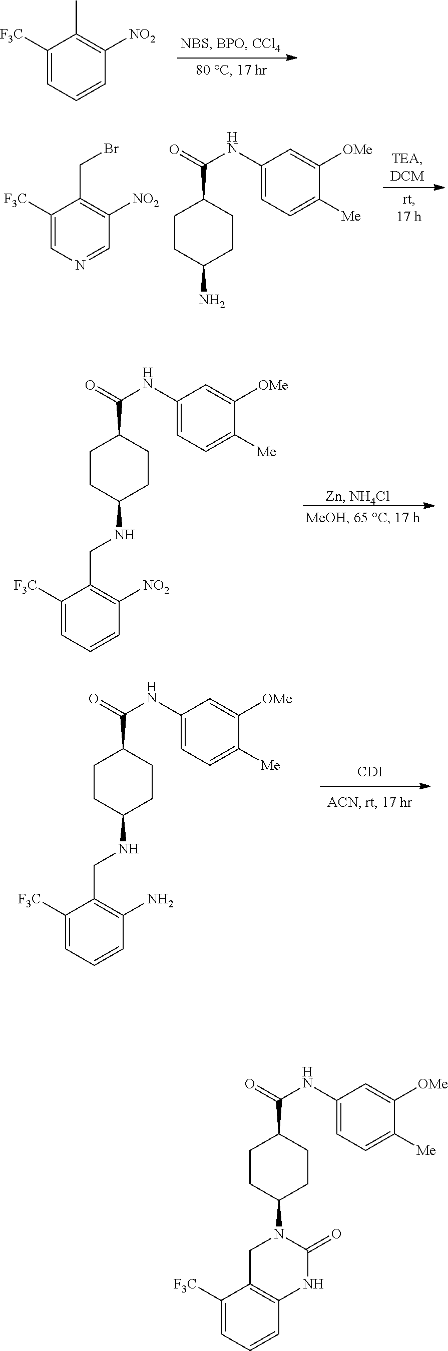

### Step 1

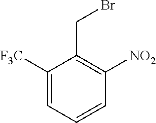 [SMILES: O=[N+]([O-])c1cccc(C(F)(F)F)c1CBr]

### 2-(bromomethyl)-1-nitro-3-(trifluoromethyl)benzene

To a solution of 2-methyl-1-nitro-3-(trifluoromethyl)benzene (1.00 g, 4.87 mmol) in CCL₄ (10 mL) was added benzoyl benzenecarboperoxoate (236 mg, 975 μmol) and NBS (0.95 g, 5.4 mmol). The mixture was stirred at 80° C. for 17 hours. One new spot was detected by TLC. The mixture was poured into water (20 mL) and extracted with EtOAc (20 mL*3). The organic phases were concentrated in vacuo and the residue was purified by silica gel chromatography (PE:EtOAc=30:1 to 1:1) to give 2-(bromomethyl)-1-nitro-3-(trifluoromethyl)benzene (1.00 g, 3.52 mmol, 72.2% yield) as a yellow oil. The isolated product was used in the subsequent step without further characterization.

### Step 2

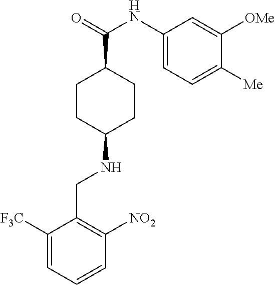 [SMILES: COc1cc(NC(=O)[C@H]2CC[C@@H](NCc3c([N+](=O)[O-])cccc3C(F)(F)F)CC2)ccc1C]

### (1s,4s)-N-(3-methoxy-4-methylphenyl)-4-(2-nitro-6 (trifluoromethyl)benzylamino)cyclohexanecarboxamide

To a solution of 4-amino-N-(3-methoxy-4-methyl-phenyl)cyclohexanecarboxamide (252 mg, 960 μmol) in DMF (5 mL) was added TEA (291 mg, 2.88 mmol, 402 μL) and 2-(bromomethyl)-1-nitro-3-(trifluoromethyl)benzene (1.00 g, 3.52 mmol). The resulting mixture was stirred at 25° C. for 17 hours. The mixture was poured into water (20 mL) and extracted with EtOAc (20 mL*3). The organic phases were concentrated in vacuo. The residue was purified by silica gel column (PE:EtOAc=30:1 to 1:1) to give (1s,4s)-N-(3-methoxy-4-methylphenyl)-4-(2-nitro-6-(trifluoromethyl)benzylamino)cyclohexanecarboxamide (400 mg, 859 μmol, 89.5% yield) as a yellow solid. LCMS ESI m/z 465.8 [M+H]⁺.

### Step 3

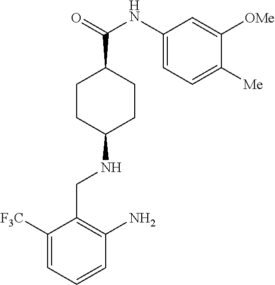 [SMILES: COc1cc(NC(=O)[C@H]2CC[C@@H](NCc3c(N)cccc3C(F)(F)F)CC2)ccc1C]

### (1s,4s)-4-(2-amino-6-(trifluoromethyl)benzylamino)-N-(3-methoxy-4-methylphenyl)cyclohexanecarboxamide

To a solution of (1s,4s)-N-(3-methoxy-4-methylphenyl)-4-(2-nitro-6-(trifluoromethyl)benzylamino)cyclohexanecarboxamide (500 mg, 1.07 mmol) in MeOH (10 mL) was added zinc powder (351 mg, 5.37 mmol) and NH₄Cl (287 mg, 5.37 mmol). The mixture was stirred at 65° C. for 17 hours. The mixture was filtered and concentrated in vacuo to give crude product, which was purified by silica gel column (PE:EtOAc=30:1 to 1:1) to give (1s,4s)-4-(2-amino-6-(trifluoromethyl)benzylamino)-N-(3-methoxy-4-methylphenyl)cyclohexanecarboxamide (400 mg, 919 μmol, 85.5% yield) as a yellow solid. LCMS ESI m/z 435.8 [M+H]⁺

### Step 4

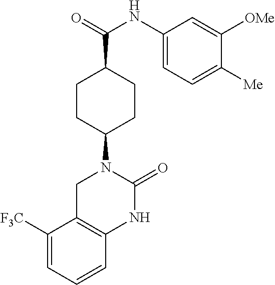 [SMILES: COc1cc(NC(=O)[C@H]2CC[C@@H](N3Cc4c(cccc4C(F)(F)F)NC3=O)CC2)ccc1C]

### (1s,4s)-N-(3-methoxy-4-methylphenyl)-4-(2-oxo-5-(trifluoromethyl)-1,2-dihydroquinazolin-3(4H)-yl)cyclohexanecarboxamide

To a solution of (1s,4s)-4-(2-amino-6-(trifluoromethyl)benzylamino)-N-(3-methoxy-4-methylphenyl)cyclohexanecarboxamide (200 mg, 459 μmol) in ACN (5 mL) was added CDI (197 mg, 1.38 mmol). The mixture was stirred at 25° C. for 17 hours. The mixture was concentrated to a residue, which was purified by prep-HPLC to give (1s,4s)-N-(3-methoxy-4-methylphenyl)-4-(2-oxo-5-(trifluoromethyl)-1,2-dihydroquinazolin-3(4H)-yl)cyclohexanecarboxamide (20 mg, 43 μmol, 9.4% yield) as a white solid. LCMS ESI m/z 461.8 [M+H]⁺. ¹HNMR (400 MHz, DMSO-d₆) δ 9.70 (s, 1H), 9.58 (s, 1H), 7.36 (t, J=7.9 Hz, 1H), 7.31 (d, J=1.5 Hz, 1H), 7.24 (d, J=7.7 Hz, 1H), 7.11-7.02 (m, 3H), 4.35 (s, 2H), 4.25-4.13 (m, 1H), 3.76 (s, 3H), 2.65-2.63 (m, 1H), 2.11-2.09 (m, 5H), 2.03-1.89 (m, 2H), 1.72-1.57 (m, 2H), 1.47-1.44 (m, 2H).

### Example 71

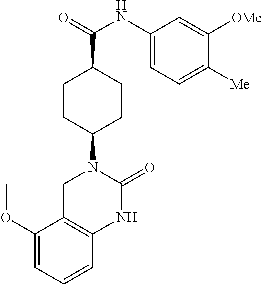 [SMILES: COc1cc(NC(=O)[C@H]2CC[C@@H](N3Cc4c(cccc4OC)NC3=O)CC2)ccc1C]

### (1s,4s)-4-(5-methoxy-2-oxo-1,2-dihydroquinazolin-3(4H)-yl)-N-(3-methoxy-4-methylphenyl)cyclohexanecarboxamide

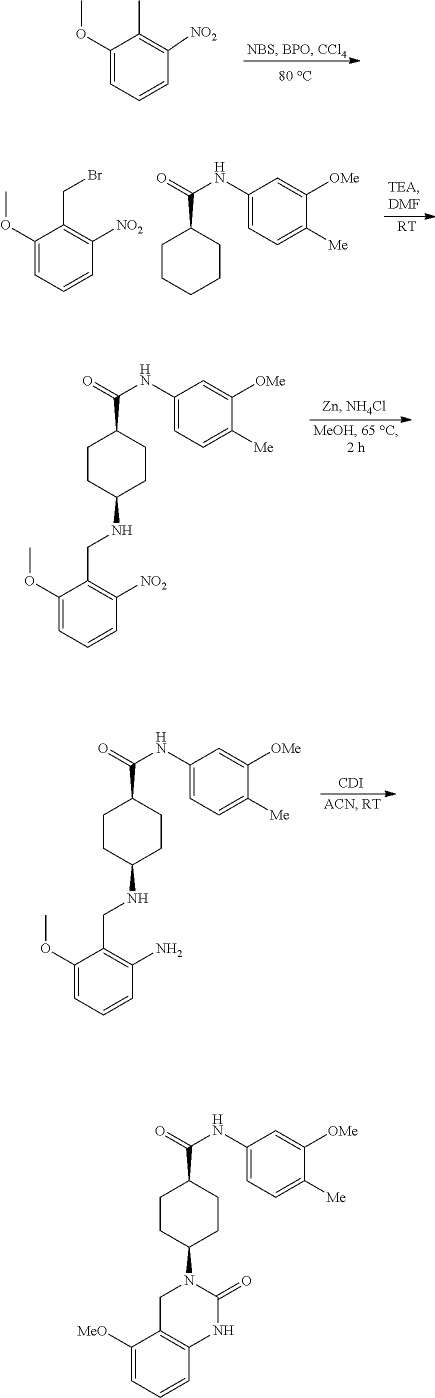

### Step 1

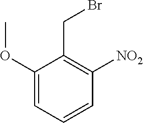

### 2-(bromomethyl)-1-methoxy-3-nitrobenzene

A mixture of 1-methoxy-2-methyl-3-nitro-benzene (1.00 g, 5.98 mmol), 1-bromopyrrolidine-2,5-dione (1.60 g, 8.97 mmol) and benzoyl benzenecarboperoxoate (145 mg, 598 μmol) in CCl₄ (15 mL) was stirred at 80° C. for 4 hours. The reaction mixture was poured into water (30 mL) and extracted with EtOAc (30 mL*3). The organic layers were dried and concentrated. The crude material was purified by silica gel chromatography to give 2-(bromomethyl)-1-methoxy-3-nitro-benzene (1.00 g, 4.06 mmol, 67.9% yield) as a yellow solid. ¹H NMR (400 MHz, CDCl₃) δ 7.54 (dd, J=8.2, 1.0 Hz, 1H), 7.43 (t, J=8.3 Hz, 1H), 7.15 (d, J=8.3 Hz, 1H), 4.83 (s, 2H), 3.98 (s, 3H).

### Step 2

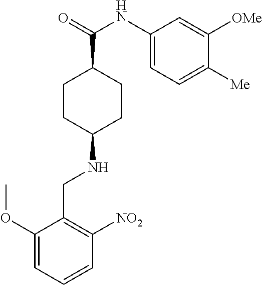 [SMILES: COc1cc(NC(=O)[C@H]2CC[C@@H](NCc3c(OC)cccc3[N+](=O)[O-])CC2)ccc1C]

### (1s,4s)-N-(3-methoxy-4-methylphenyl)-4-(2-methoxy-6-nitrobenzylamino)cyclohexanecarboxamide

A solution of 2-(bromomethyl)-1-methoxy-3-nitro-benzene (100 mg, 406 μmol), 4-amino-N-(3-methoxy-4-methyl-phenyl)cyclohexanecarboxamide (107 mg, 406 μmol) and N, N-diethylethanamine (123 mg, 1.22 mmol, 170 μL) in DMF (2 mL) was stirred at 25° C. for 3 hours. The reaction mixture was poured into water (20 mL) and extracted with EtOAc (30 mL). The organic layer was dried and concentrated to the crude product (1s,4s)-N-(3-methoxy-4-methylphenyl)-4-(2-methoxy-6-nitrobenzylamino)cyclohexanecarboxamide (160 mg, 374 μmol, 92.1% yield) as a yellow oil. LCMS (ESI) m/z: 428.2 [M+H]⁺.

### Step 3 and 4

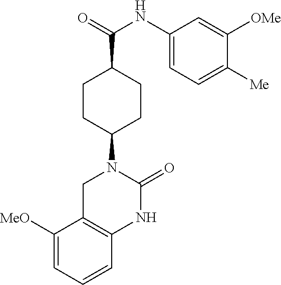 [SMILES: COc1cc(NC(=O)[C@H]2CC[C@@H](N3Cc4c(cccc4OC)NC3=O)CC2)ccc1C]

### (1s,4s)-4-(5-methoxy-2-oxo-1,2-dihydroquinazolin-3(4H)-yl)-N-(3-methoxy-4-methylphenyl)cyclohexanecarboxamide

Following the general procedure above in Example 66, but starting with (1s,4s)-N-(3-methoxy-4-methylphenyl)-4-(2-methoxy-6-nitrobenzylamino)cyclohexanecarboxamide gave the title compound as a white solid. LCMS (ESI) m/z: 424.2 [M+H]⁺. ¹H NMR (400 MHz, CDCl₃) δ 7.36 (s, 1H), 7.24 (s, 1H), 7.08 (t, J=8.4 Hz, 2H), 6.81 (dd, J=8.0, 1.9 Hz, 1H), 6.55 (s, 1H), 6.44 (d, J=8.2 Hz, 1H), 6.23 (d, J=7.8 Hz, 1H), 4.46-4.38 (m, 1H), 4.32 (s, 2H), 3.87 (s, 3H), 3.79 (s, 3H), 2.66-2.64 (m, 1H), 2.28-2.24 (m, 2H), 2.19 (s, 3H), 2.10-1.97 (m, 2H), 1.86-1.70 (m, 2H), 1.68-1.53 (m, 2H).

### Example 72

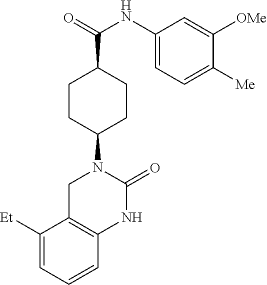 [SMILES: CCc1cccc2c1CN([C@H]1CC[C@@H](C(=O)Nc3ccc(C)c(OC)c3)CC1)C(=O)N2]

### (1s,4s)-4-(5-ethyl-2-oxo-1,2-dihydroquinazolin-3(4H)-yl)-N-(3-methoxy-4-methylphenyl)cyclohexanecarboxamide

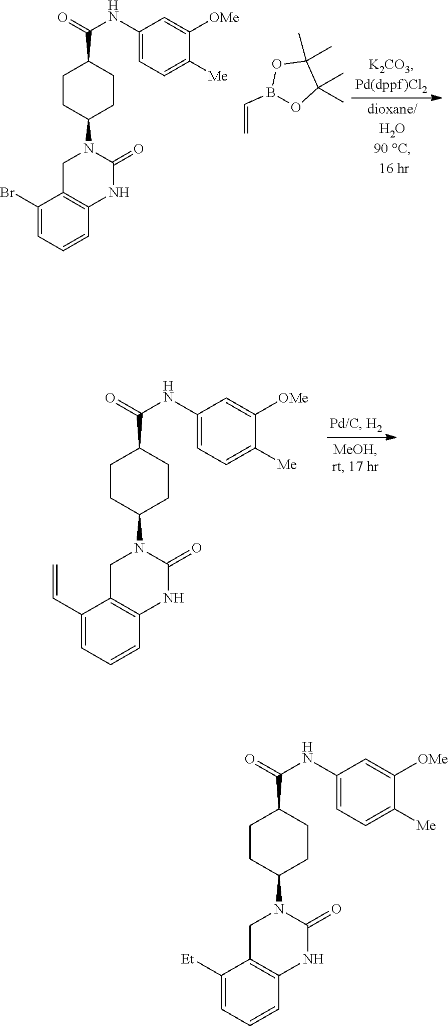

### Step 1

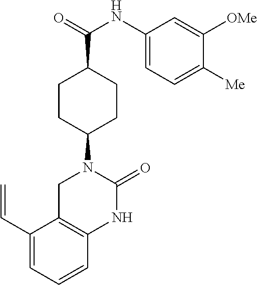 [SMILES: C=Cc1cccc2c1CN([C@H]1CC[C@@H](C(=O)Nc3ccc(C)c(OC)c3)CC1)C(=O)N2]

### (1s,4s)-N-(3-methoxy-4-methylphenyl)-4-(2-oxo-5-vinyl-1,2-dihydroquinazolin-3(4H)-yl)cyclohexanecarboxamide

To a 4-(5-bromo-2-oxo-1,4-dihydroquinazolin-3-yl)-N-(3-methoxy-4-methyl-phenyl)cyclohexanecarboxamide (150 mg, 318 μmol) in dioxane/H₂O (5/1, 12 mL) was added 4,4,5,5-tetramethyl-2-vinyl-1,3,2-dioxaborolane (154 mg, 97.8 μmol), Pd(dppf)Cl₂ (23 mg, 32 μmol) and Cs₂CO₃ (207 mg, 635 μmol). The mixture was stirred at 100° C. for 17 hours under nitrogen. The mixture was poured into water (20 mL) and extracted with EtOAc (10 mL*3). The organic layers were dried and concentrated to a crude, which was purified by silica gel chromatography (PE:EtOAc=30:1 to 1:1) to give (1s,4s)-N-(3-methoxy-4-methylphenyl)-4-(2-oxo-5-vinyl-1,2-dihydroquinazolin-3(4H)-yl)cyclohexanecarboxamide (120 mg, 286 μmol, 67.6% yield) as a yellow solid. LCMS ESI m/z 419.9 [M+H]⁺.

### Step 2

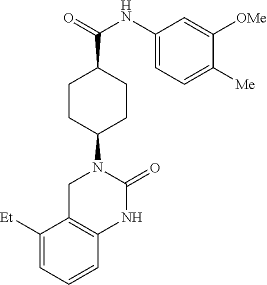 [SMILES: CCc1cccc2c1CN([C@H]1CC[C@@H](C(=O)Nc3ccc(C)c(OC)c3)CC1)C(=O)N2]

### (1s,4s)-4-(5-ethyl-2-oxo-1,2-dihydroquinazolin-3(4H)-yl)-N-(3-methoxy-4-methylphenyl)cyclohexanecarboxamide

To a solution of (1s,4s)-N-(3-methoxy-4-methylphenyl)-4-(2-oxo-5-vinyl-1,2-dihydroquinazolin-3(4H)-yl)cyclohexanecarboxamide (120 mg, 286 μmol) in MeOH (2 mL) was added Pd/C (10 mg). The resulting mixture was stirred at 25° C. under H₂ for 17 hours. The mixture was filtered and concentrated in vacuo. The residue was purified by prep-HPLC to give (1s,4s)-4-(5-ethyl-2-oxo-1,2-dihydroquinazolin-3(4H)-yl)-N-(3-methoxy-4-methylphenyl)cyclohexanecarboxamide (10.5 mg, 24.9 μmol, 8.71% yield) as a white solid. LCMS ESI m/z 422.3 [M+H]⁺. ¹HNMR (400 MHz, DMSO-d₆) δ 9.69 (s, 1H), 9.08 (s, 1H), 7.30 (d, J=1.7 Hz, 1H), 7.13-6.98 (m, 3H), 6.72 (d, J=7.3 Hz, 1H), 6.62 (d, J=7.8 Hz, 1H), 4.24 (s, 2H), 4.22-4.20 (m, 1H), 3.76 (s, 3H), 2.66-2.64 (m, 1H), 2.54-2.52 (m, 1H), 2.48-2.46 (m, 1H), 2.17-1.93 (m, 7H), 1.73-1.59 (m, 2H), 1.48-1.44 (m, 2H), 1.12 (t, J=7.5 Hz, 3H).

### Example 73

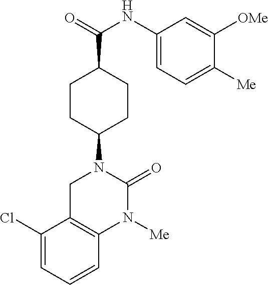 [SMILES: COc1cc(NC(=O)[C@H]2CC[C@@H](N3Cc4c(Cl)cccc4N(C)C3=O)CC2)ccc1C]

### (1s,4s)-4-(5-chloro-1-methyl-2-oxo-1,2-dihydroquinazolin-3(4H)-yl)-N-(3-methoxy-4-methylphenyl)cyclohexanecarboxamide

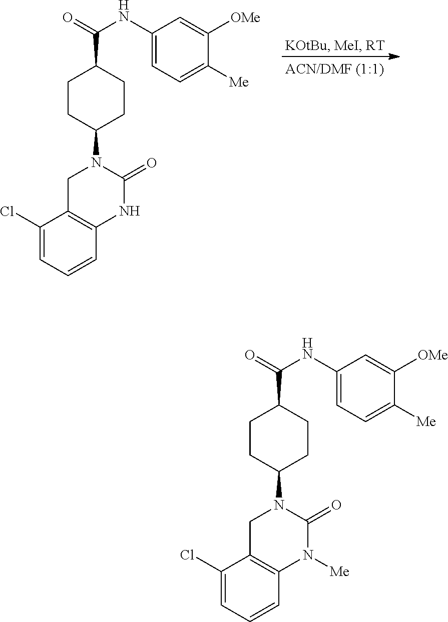 [SMILES: COc1cc(NC(=O)[C@H]2CC[C@@H](N3Cc4c(Cl)cccc4NC3=O)CC2)ccc1C ; COc1cc(NC(=O)[C@H]2CC[C@@H](N3Cc4c(Cl)cccc4N(C)C3=O)CC2)ccc1C]

### Step 1

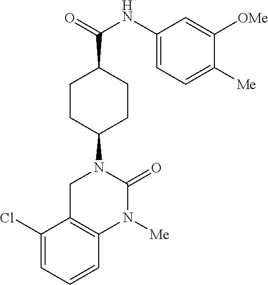 [SMILES: COc1cc(NC(=O)[C@H]2CC[C@@H](N3Cc4c(Cl)cccc4N(C)C3=O)CC2)ccc1C]

### (1s,4s)-4-(5-chloro-1-methyl-2-oxo-1,2-dihydroquinazolin-3(4H)-yl)-N-(3-methoxy-4-methylphenyl)cyclohexanecarboxamide

A mixture of (1s,4s)-4-(5-chloro-2-oxo-1,2-dihydroquinazolin-3(4H)-yl)-N-(3-methoxy-4-methylphenyl)cyclohexanecarboxamide (Example 68) (50.0 mg, 117 μmol), potassium; 2-methylpropan-2-olate (10.5 mg, 93.5 μmol) and iodomethane (13.3 mg, 93.5 μmol, 5.82 μL) in ACN/DMF (1/1, 5 mL) was stirred at room temperature for 1 hour. The reaction mixture was diluted with water (20 mL) and extracted with EtOAc (30 mL). The organic layer was dried over anhydrous Na₂SO₄, filtered and concentrated to get crude product, which was purified by prep-HPLC to give (1s,4s)-4-(5-chloro-1-methyl-2-oxo-1,2-dihydroquinazolin-3(4H)-yl)-N-(3-methoxy-4-methylphenyl)cyclohexanecarboxamide (6.0 mg, 10% yield) as a white solid. LCMS (ESI) m/z: 443.8 [M+H]⁺. ¹H NMR (400 MHz, DMSO-d₆) δ 9.70 (s, 1H), 7.32-7.27 (m, 2H), 7.13-7.03 (m, 2H), 7.02 (d, J=8.5 Hz, 1H), 6.92 (d, J=8.1 Hz, 1H), 4.31 (s, 2H), 4.19-4.16 (m, 1H), 3.76 (s, 3H), 3.18 (s, 3H), 2.66-2.64 (m, 1H), 2.12-2.09 (m, 5H), 2.04-1.93 (m, 2H), 1.72-1.61 (m, 2H), 1.51-1.43 (m, 2H).

### Example 74

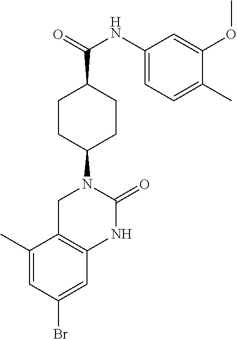

### (1s,4s)-4-(7-bromo-5-methyl-2-oxo-1,2-dihydroquinazolin-3(4H)-yl)-N-(3-methoxy-4-methylphenyl)cyclohexanecarboxamide

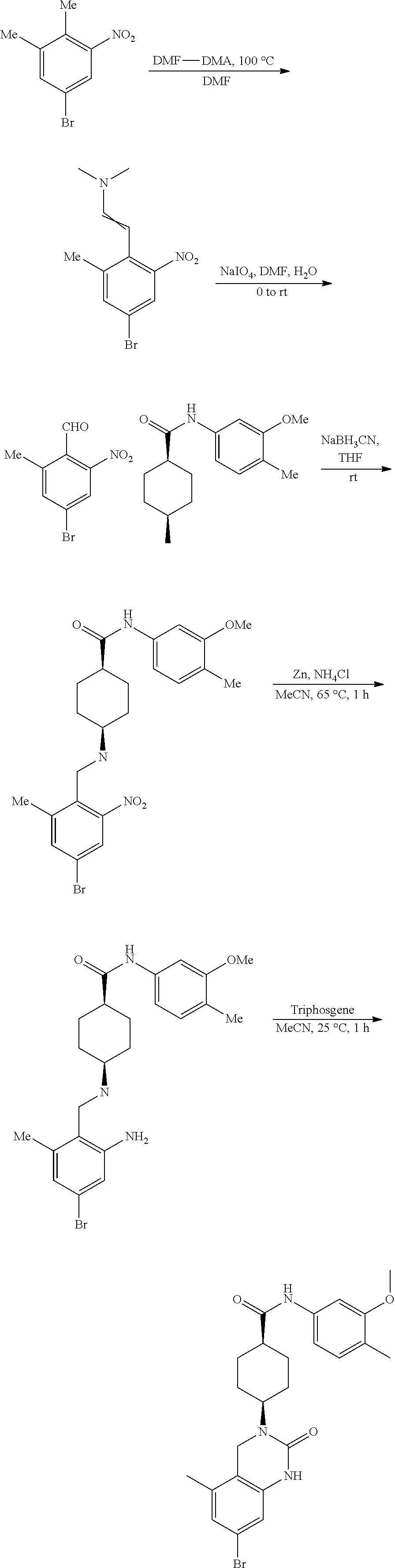

### Step 1

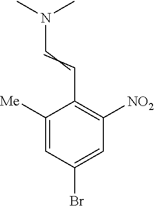 [SMILES: Cc1cc(Br)cc([N+](=O)[O-])c1C=CN(C)C]

### 2-(4-bromo-2-methyl-6-nitrophenyl)-N,N-dimethylethenamine

A solution of 5-bromo-1,2-dimethyl-3-nitro-benzene (8.8 g, 38 mmol) and 1,1-dimethoxy-N,N-dimethyl-methanamine (9.12 g, 76.5 mmol, 10.2 mL) in DMF (40 mL) was stirred for 16 hours at 100° C. The reaction mixture was directly used for the next step.

### Step 2

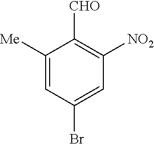 [SMILES: Cc1cc(Br)cc([N+](=O)[O-])c1C=O]

### 4-bromo-2-methyl-6-nitrobenzaldehyde

To a solution of NaIO₄ (32.7 g, 153 mmol) in water (10 mL) and DMF (10 mL) was added 2-(4-bromo-2-methyl-6-nitrophenyl)-N,N-dimethylethenamine (10.9 g, 38.2 mmol) at 0-10° C. The mixture was stirred for 5 hours at 25° C. EtOAc (250 mL) was added to the reaction mixture. The solution was washed with water (3×150 mL). The combined organic extracts were dried over Na₂SO₄ and evaporated in vacuo. The residue was purified by flash chromatography on silica gel (0-10% EtOAc in PE) to get 4-bromo-2-methyl-6-nitro-benzaldehyde (2.00 g, 8.20 mmol, 21.4% yield) as a white solid (TLC:PE:EtOAc=5:1, R_{f}=0.5). ¹H NMR (400 MHz, CDCl₃) δ 10.28 (s, 1H), 8.10 (d, J=1.6 Hz, 1H), 7.74 (d, J=0.8 Hz, 1H), 2.50 (s, 3H).

### Step 3

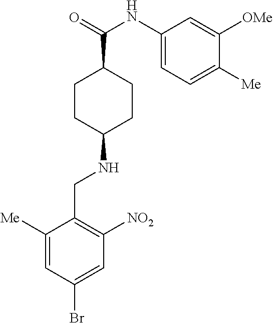 [SMILES: COc1cc(NC(=O)[C@H]2CC[C@@H](NCc3c(C)cc(Br)cc3[N+](=O)[O-])CC2)ccc1C]

### (1s,4s)-4-(4-bromo-2-methyl-6-nitrobenzylamino)-N-(3-methoxy-4-methylphenyl)cyclohexanecarboxamide

A solution of 4-bromo-2-methyl-6-nitro-benzaldehyde (233 mg, 953 μmol) and 4-amino-N-(3-methoxy-4-methyl-phenyl)cyclohexanecarboxamide (0.10 g, 380 μmol) in THF (3 mL) was stirred for 16 hours at 25° C. Sodium cyanoborohydride (120 mg, 1.91 mmol) was added to the reaction mixture. The resulting mixture was stirred for 1 hour at 25° C. The reaction mixture was worked up with EtOAc (50 mL) and water (3×50 mL). The combined organic extracts were dried over Na₂SO₄ and evaporated in vacuo. The residue was purified by flash chromatography on silica gel (0-40% EtOAc in PE) to get (1s,4s)-4-(4-bromo-2-methyl-6-nitrobenzylamino)-N-(3-methoxy-4-methylphenyl)cyclohexanecarboxamide (50 mg, 100 μmol, 27% yield) as a white solid (TLC:PE/EtOAc=2/1, R_{f}=0.3). LCMS (ESI) m/z: 489.7 [M+H]⁺.

### Step 4

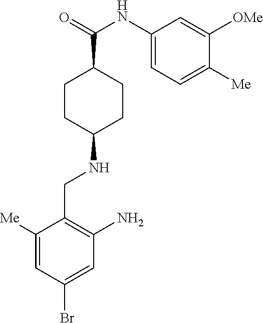 [SMILES: COc1cc(NC(=O)[C@H]2CC[C@@H](NCc3c(C)cc(Br)cc3N)CC2)ccc1C]

### (1s,4s)-4-(2-amino-4-bromo-6-methylbenzylamino)-N-(3-methoxy-4-methylphenyl)cyclohexanecarboxamide

A solution of (1s,4s)-4-(4-bromo-2-methyl-6-nitrobenzylamino)-N-(3-methoxy-4-methylphenyl)cyclohexanecarboxamide (50 mg, 100 μmol), NH₄Cl (54 mg, 1.0 mmol) and zinc powder (66 mg, 1.0 mmol) in MeOH (10 mL) was stirred for 1 hour at 65° C. The reaction mixture was filtered through a celite pad and the filtrate was concentrated to get (1s,4s)-4-(2-amino-4-bromo-6-methylbenzylamino)-N-(3-methoxy-4-methylphenyl)cyclohexanecarboxamide (0.046 g, 100 μmol, 98% yield) as a white solid. LCMS (ESI) m/z: 459.8 [M+H]⁺.

### Step 5

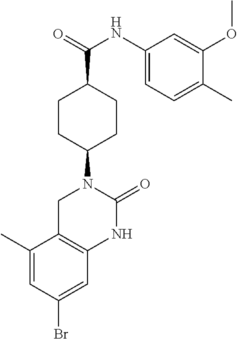

### (1s,4s)-4-(7-bromo-5-methyl-2-oxo-1,2-dihydroquinazolin-3(4H)-yl)-N-(3-methoxy-4-methylphenyl)cyclohexanecarboxamide

A solution of (1s,4s)-4-(2-amino-4-bromo-6-methylbenzylamino)-N-(3-methoxy-4-methylphenyl)cyclohexanecarboxamide (0.060 g, 130 μmol) and bis(trichloromethyl)carbonate (116 mg, 391 μmol) in CH₃CN (10 mL) was stirred for 1 hour at 25° C. The reaction mixture was diluted in EtOAc (50 mL) and washed with water (3×50 mL). The combined organic extracts were dried over Na₂SO₄ and evaporated in vacuo. The residue was purified by Prep-HPLC to get (1s,4s)-4-(7-bromo-5-methyl-2-oxo-1,2-dihydroquinazolin-3(4H)-yl)-N-(3-methoxy-4-methylphenyl)cyclohexanecarboxamide (0.0134 g, 27.6 μmol, 21.1% yield) as a white solid. LCMS (ESI) m/z: 486.1 [M+H]⁺. ¹H NMR (400 MHz, MeOD-d₄) δ 7.25 (d, J=1.6 Hz, 1H), 7.05-7.02 (m, 1H), 6.99-6.96 (m, 1H), 6.92 (d, J=1.2 Hz, 1H), 6.78 (d, J=1.6 Hz, 1H), 4.34-4.30 (m, 3H), 3.82 (s, 3H), 2.71-2.68 (m, 1H), 2.27-2.09 (m, 10H), 1.85-1.70 (m, 2H), 1.65-1.55 (m, 2H).

### Example 75

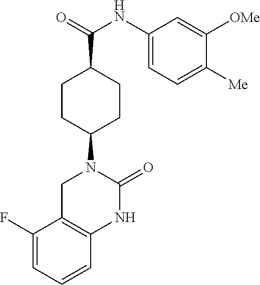 [SMILES: COc1cc(NC(=O)[C@H]2CC[C@@H](N3Cc4c(F)cccc4NC3=O)CC2)ccc1C]

### (1s,4s)-4-(5-fluoro-2-oxo-1,2-dihydroquinazolin-3(4H)-yl)-N-(3-methoxy-4-methylphenyl)cyclohexanecarboxamide

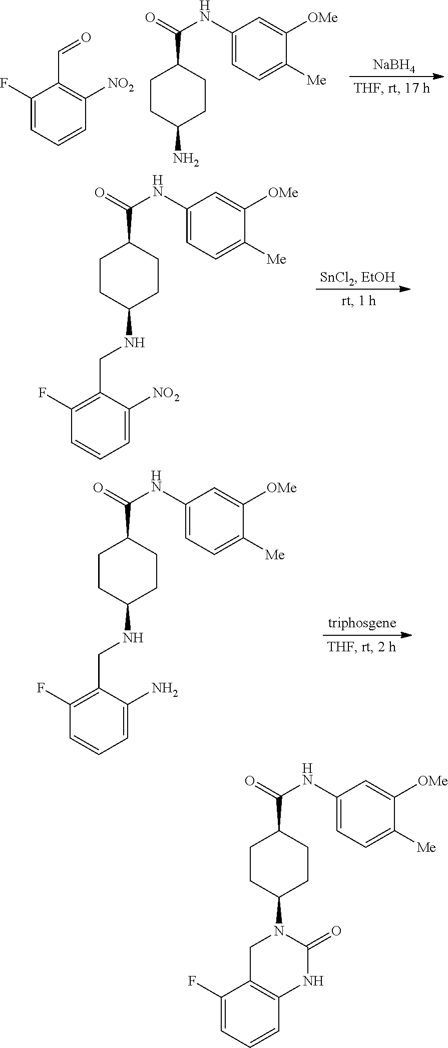

### Step 1

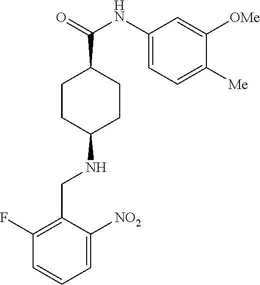 [SMILES: COc1cc(NC(=O)[C@H]2CC[C@@H](NCc3c(F)cccc3[N+](=O)[O-])CC2)ccc1C]

### (1s,4s)-4-(2-fluoro-6-nitrobenzylamino)-N-(3-methoxy-4-methylphenyl)cyclohexanecarboxamide

A solution of 2-fluoro-6-nitro-benzaldehyde (516 mg, 3.05 mmol) and 4-amino-N-(3-methoxy-4-methyl-phenyl)cyclohexanecarboxamide (0.40 g, 1.52 mmol) in THF (10 mL) was stirred for 16 hours at 25° C. Sodium borohydride (116 mg, 3.05 mmol) was added to the mixture, and the resulting mixture was stirred for another 1 hour at 25° C. The reaction mixture was quenched by sat. NH₄Cl (30 mL), and the mixture was worked up with EtOAc (50 mL) and H₂O (3×50 mL). The combined organic extracts were dried over Na₂SO₄ and evaporated in vacuo. The residue was purified by flash chromatography on silica gel (0-30% EtOAc in PE) to get (1s,4s)-4-(2-fluoro-6-nitrobenzylamino)-N-(3-methoxy-4-methylphenyl)cyclohexanecarboxamide (0.14 g, 340 μmol, 22% yield) as a white solid. LCMS ESI m/z 416.2 [M+H]⁺

### Step 2

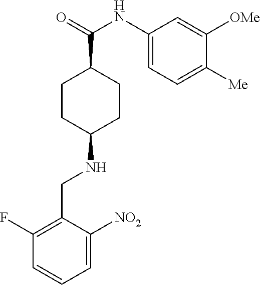 [SMILES: COc1cc(NC(=O)[C@H]2CC[C@@H](NCc3c(F)cccc3[N+](=O)[O-])CC2)ccc1C]

### (1s,4s)-4-(2-amino-6-fluorobenzylamino)-N-(3-methoxy-4-methylphenyl)cyclohexanecarboxamide

A solution of (1s,4s)-4-(2-fluoro-6-nitrobenzylamino)-N-(3-methoxy-4-methylphenyl)cyclohexanecarboxamide (0.12 g, 290 μmol) and SnCl₂ (165 mg, 867 μmol) in EtOH (5 mL) was stirred for 1 h at 25° C. The reaction mixture was filtered through a celite pad and the filtrate was concentrated to get (1s,4s)-4-(2-amino-6-fluorobenzylamino)-N-(3-methoxy-4-methylphenyl)cyclohexanecarboxamide (0.10 g, 260 μmol, 90% yield) as a white solid. LCMS ESI m/z 385.9 [M+H]⁺.

### Step 3

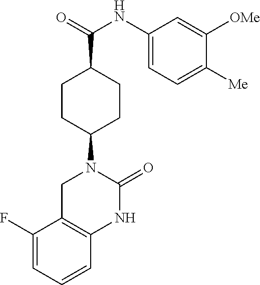 [SMILES: COc1cc(NC(=O)[C@H]2CC[C@@H](N3Cc4c(F)cccc4NC3=O)CC2)ccc1C]

### (1s,4s)-4-(5-fluoro-2-oxo-1,2-dihydroquinazolin-3(4H)-yl)-N-(3-methoxy-4-methylphenyl)cyclohexanecarboxamide

To a solution of (1s,4s)-4-(2-amino-6-fluorobenzylamino)-N-(3-methoxy-4-methylphenyl)cyclohexanecarboxamide (25 mg, 65 μmol) and triphosgene (38 mg, 130 μmol) in THF (2 mL) was stirred at 25° C. for 2 hours. The mixture was quenched with water (10 mL) and extracted with EtOAc (10 mL*2). The organic layers were dried and concentrated. The residue was purified by prep-HPLC to give (1s,4s)-4-(5-fluoro-2-oxo-1,2-dihydroquinazolin-3(4H)-yl)-N-(3-methoxy-4-methylphenyl)cyclohexanecarboxamide (10 mg, 23 μmol, 35% yield) as a white solid. LCMS ESI m/z 411.9 [M+H]⁺. ¹H NMR (400 MHz, DMSO-d₆) δ 9.69 (s, 1H), 9.41 (s, 1H), 7.29 (d, J=1.6 Hz, 1H), 7.19-7.08 (m, 2H), 7.02 (d, J=8.2 Hz, 1H), 6.70 (t, J=8.8 Hz, 1H), 6.59 (d, J=8.0 Hz, 1H), 4.35-4.16 (m, 3H), 3.76 (s, 3H), 2.66-2.64 (m, 1H), 2.13-2.09 (m, 5H), 2.04-1.91 (m, 2H), 1.74-1.59 (m, 2H), 1.47-1.43 (m, 2H).

### Example 76

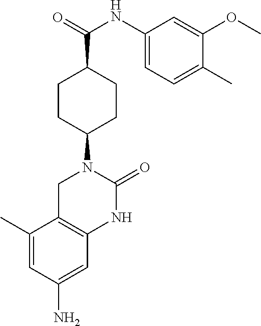

### (1s,4s)-4-(7-amino-5-methyl-2-oxo-1,2-dihydroquinazolin-3(4H)-yl)-N-(3-methoxy-4-methylphenyl)cyclohexanecarboxamide

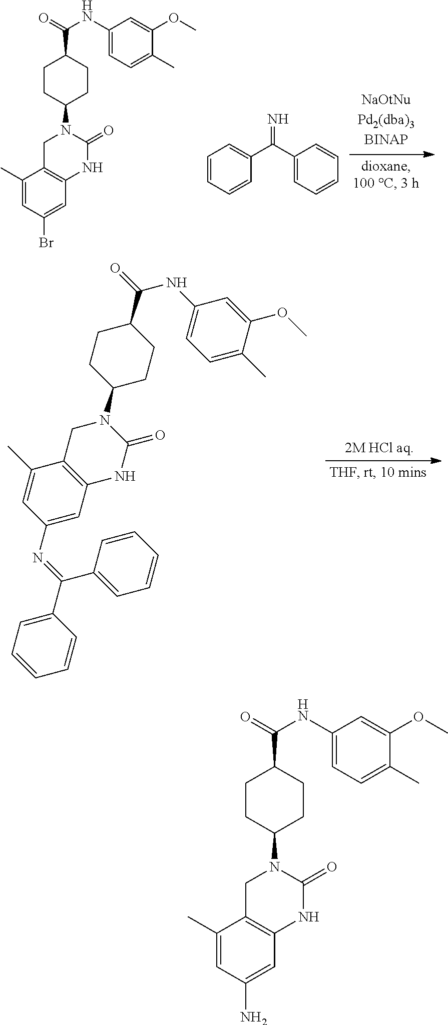

### Step 1

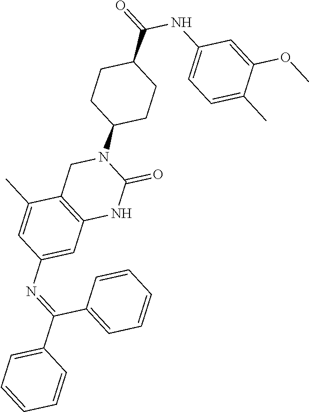 [SMILES: COc1cc(NC(=O)[C@H]2CC[C@@H](N3Cc4c(C)cc(N=C(c5ccccc5)c5ccccc5)cc4NC3=O)CC2)ccc1C]

### (1s,4s)-4-(7-(diphenylmethyleneamino)-5-methyl-2-oxo-1,2-dihydroquinazolin-3(4H)-yl)-N-(3-methoxy-4-methylphenyl)cyclohexanecarboxamide

To a solution of 4-(7-bromo-5-methyl-2-oxo-1,4-dihydroquinazolin-3-yl)-N-(3-methoxy-4-methyl-phenyl)cyclohexanecarboxamide (100 mg, 206 μmol) and diphenylmethanimine (75 mg, 410 μmol) in dioxane (2 mL) was added Pd₂(dba)₃ (19 mg, 21 μmol), BINAP (26 mg, 41 μmol) and sodium tert-butoxide (59 mg, 620 μmol). The mixture was stirred at 100° C. for 3 hours. The reaction mixture was filtered and concentrated to give a crude oil. The crude product was purified by silica gel chromatography to give (1s,4s)-4-(7-(diphenylmethyleneamino)-5-methyl-2-oxo-1,2-dihydroquinazolin-3(4H)-yl)-N-(3-methoxy-4-methylphenyl)cyclohexanecarboxamide (60 mg, 72 μmol, 35% yield, 70% purity) as a yellow solid. LCMS ESI m/z: 587.3 [M+H]⁺.

### Step 2

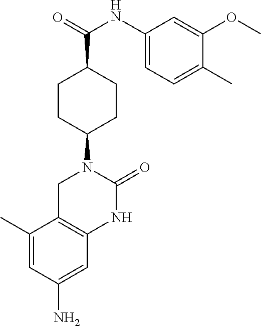

### (1s,4s)-4-(7-amino-5-methyl-2-oxo-1,2-dihydroquinazolin-3(4H)-yl)-N-(3-methoxy-4-methylphenyl)cyclohexanecarboxamide

To a solution of (1s,4s)-4-(7-(diphenylmethyleneamino)-5-methyl-2-oxo-1,2-dihydroquinazolin-3(4H)-yl)-N-(3-methoxy-4-methylphenyl)cyclohexanecarboxamide (45 mg, 77 μmol) in THF (2 mL) was added 2M HCl aq. (0.1 mL). The resulting solution was stirred for 10 minutes at room temperature, then partitioned between water and EtOAc. The aqueous layer was separated, basified to pH=9 with concentrated aqueous ammonia, extracted with DCM (3×10 mL). The combined organics were dried, filtered and concentrated in vacuo to give a yellow solid, which was purified by prep-HPLC to give (1s,4s)-4-(7-amino-5-methyl-2-oxo-1,2-dihydroquinazolin-3(4H)-yl)-N-(3-methoxy-4-methylphenyl)cyclohexanecarboxamide (6.7 mg, 16 μmol, 20% yield) as a white solid. LCMS ESI m/z: 422.9 [M+H]⁺. ¹H NMR (400 MHz, DMSO-d₆) δ 9.68 (s, 1H), 8.79 (s, 1H), 7.29 (d, J=1.6 Hz, 1H), 7.11 (dd, J=8.0, 1.8 Hz, 1H), 7.02 (d, J=8.1 Hz, 1H), 5.95 (d, J=1.5 Hz, 1H), 5.85 (d, J=1.9 Hz, 1H), 4.85 (s, 2H), 4.21-4.19 (m, 1H), 4.07 (s, 2H), 3.76 (s, 3H), 2.66-2.64 (m, 1H), 2.13-2.09 (m, 5H), 2.04-1.92 (m, 5H), 1.68-2.63 (m, 2H), 1.46-1.42 (m, 2H).

### Example 77

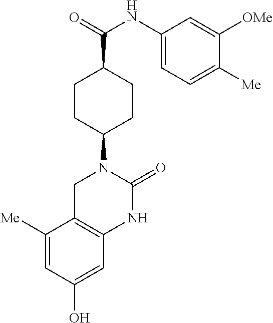 [SMILES: COc1cc(NC(=O)[C@H]2CC[C@@H](N3Cc4c(C)cc(O)cc4NC3=O)CC2)ccc1C]

### (1s,4s)-4-(7-hydroxy-5-methyl-2-oxo-1,2-dihydroquinazolin-3(4H)-yl)-N-(3-methoxy-4-methylphenyl)cyclohexanecarboxamide

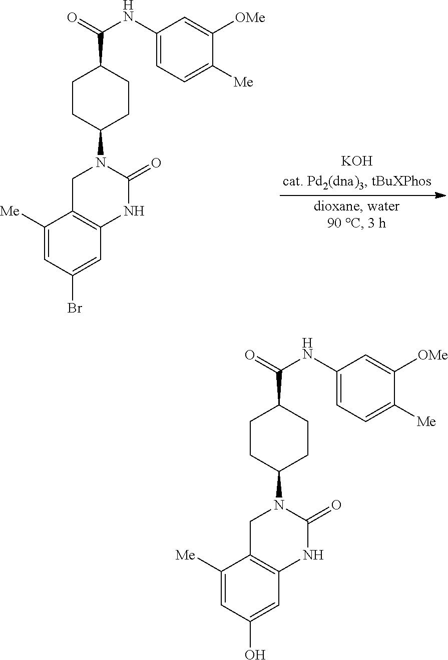 [SMILES: COc1cc(NC(=O)[C@H]2CC[C@@H](N3Cc4c(C)cc(Br)cc4NC3=O)CC2)ccc1C ; COc1cc(NC(=O)[C@H]2CC[C@@H](N3Cc4c(C)cc(O)cc4NC3=O)CC2)ccc1C]

### Step 1

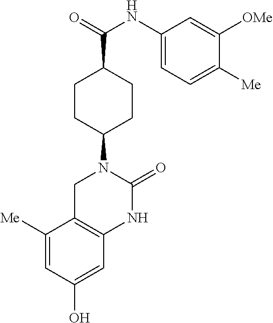 [SMILES: COc1cc(NC(=O)[C@H]2CC[C@@H](N3Cc4c(C)cc(O)cc4NC3=O)CC2)ccc1C]

### (1s,4s)-4-(7-hydroxy-5-methyl-2-oxo-1,2-dihydroquinazolin-3(4H)-yl)-N-(3-methoxy-4-methylphenyl)cyclohexanecarboxamide

A mixture of (1s,4s)-4-(7-bromo-5-methyl-2-oxo-1,2-dihydroquinazolin-3(4H)-yl)-N-(3-methoxy-4-methylphenyl)cyclohexanecarboxamide (100 mg, 205.59 μmol), KOH (23 mg, 410 μmol). Pd₂(dba)₃ (19 mg, 21 μmol) and t-BuXPhos (8.7 mg, 21 μmol) in dioxane/H₂O (4:1, 5 mL) was stirred at 90° C. for 3 hours under N₂. The reaction mixture was diluted with water (20 mL) and extracted with EtOAc (30 mL). The organic layer was dried over anhydrous Na₂SO₄, filtered and concentrated to get crude product, which was purified by prep-HPLC to give (1s,4s)-4-(7-hydroxy-5-methyl-2-oxo-1,2-dihydroquinazolin-3(4H)-yl)-N-(3-methoxy-4-methylphenyl)cyclohexanecarboxamide (13 mg, 14% yield) as a white solid. LCMS (ESI) m/z: 424.2 [M+H]⁺. ¹H NMR (400 MHz, DMSO-d₆) δ 9.68 (s, 1H), 9.14 (s, 1H), 8.91 (s, 1H), 7.28 (d, J=1.5 Hz, 1H), 7.11 (dd, J=8.0, 1.7 Hz, 1H), 7.02 (d, J=8.1 Hz, 1H), 6.12 (d, J=1.8 Hz, 1H), 6.07 (d, J=2.1 Hz, 1H), 4.22-4.16 (m, 1H), 4.11 (s, 2H), 3.75 (s, 3H), 2.65-2.63 (m, 1H), 2.13-2.10 (m, 2H), 2.08 (s, 3H), 2.05 (s, 3H), 2.01-1.91 (m, 2H), 1.68-1.60 (m, 2H), 1.45-1.43 (m, 2H).

### Example 78

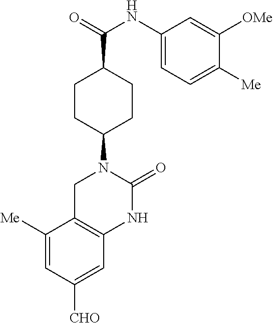 [SMILES: COc1cc(NC(=O)[C@H]2CC[C@@H](N3Cc4c(C)cc(C=O)cc4NC3=O)CC2)ccc1C]

### (1s,4s)-4-(7-formyl-5-methyl-2-oxo-1,2-dihydroquinazolin-3(4H)-yl)-N-(3-methoxy-4-methylphenyl)cyclohexanecarboxamide

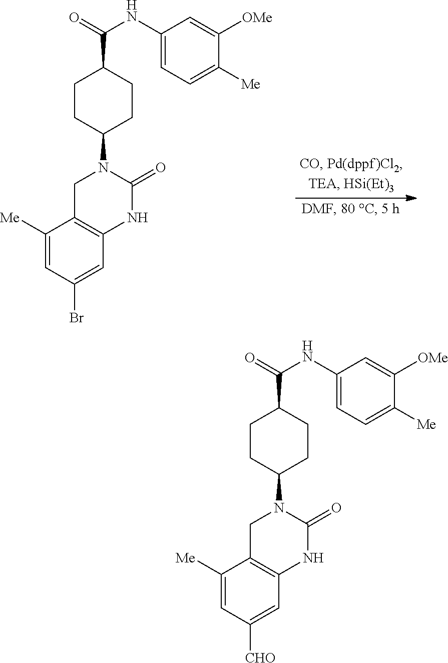 [SMILES: COc1cc(NC(=O)[C@H]2CC[C@@H](N3Cc4c(C)cc(Br)cc4NC3=O)CC2)ccc1C ; COc1cc(NC(=O)[C@H]2CC[C@@H](N3Cc4c(C)cc(C=O)cc4NC3=O)CC2)ccc1C]

### Step 1

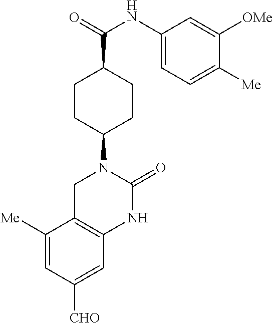 [SMILES: COc1cc(NC(=O)[C@H]2CC[C@@H](N3Cc4c(C)cc(C=O)cc4NC3=O)CC2)ccc1C]

### (1s,4s)-4-(7-formyl-5-methyl-2-oxo-1,2-dihydroquinazolin-3(4H)-yl)-N-(3-methoxy-4-methylphenyl)cyclohexanecarboxamide

A mixture of 4-(7-bromo-5-methyl-2-oxo-1,4-dihydroquinazolin-3-yl)-N-(3-methoxy-4-methyl-phenyl)cyclohexanecarboxamide (0.17 g, 350 μmol), N,N-diethylethanamine (71 mg, 700 μmol, 97 μL), HSi(Et)₃ (406 mg, 3.50 mmol) and Pd(dppf)Cl₂ (26 mg, 35 μmol) in DMF (10 mL) was stirred for 5 hours at 80° C. under carbon monoxide atmosphere (balloon). The reaction mixture was filtered and concentrated. The residue was purified by flash chromatography on silica gel (0-10% MeOH in DCM) and Prep-HPLC to get (1s,4s)-4-(7-formyl-5-methyl-2-oxo-1,2-dihydroquinazolin-3(4H)-yl)-N-(3-methoxy-4-methylphenyl)cyclohexanecarboxamide (0.058 g, 130 μmol, 38% yield) as a white solid. LCMSESI m/z: 436.3 [M+H]⁺. ¹H NMR (400 MHz, MeOD-d₄) δ 9.84 (s, 1H), 7.32 (s, 1H), 7.26 (d, J=1.6 Hz, 1H), 7.12 (s, 1H), 7.04 (d, J=8.0 Hz, 1H), 6.97 (dd, J=8.0, 2.0 Hz, 1H), 4.46 (s, 2H), 4.40-4.30 (m, 1H), 3.83 (s, 3H), 2.73-2.71 (m, 1H), 2.34 (s, 3H), 2.29-2.16 (m, 4H), 2.14 (s, 3H), 1.85-1.73 (m, 2H), 1.63-1.55 (m, 2H).

### Example 79

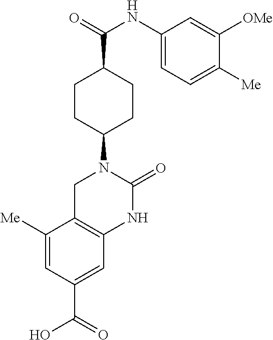 [SMILES: COc1cc(NC(=O)[C@H]2CC[C@@H](N3Cc4c(C)cc(C(=O)O)cc4NC3=O)CC2)ccc1C]

### 3-((1s,4s)-4-(3-methoxy-4-methylphenylcarbamoyl)cyclohexyl)-5-methyl-2-oxo-1,2,3,4-tetrahydroquinazoline-7-carboxylic Acid

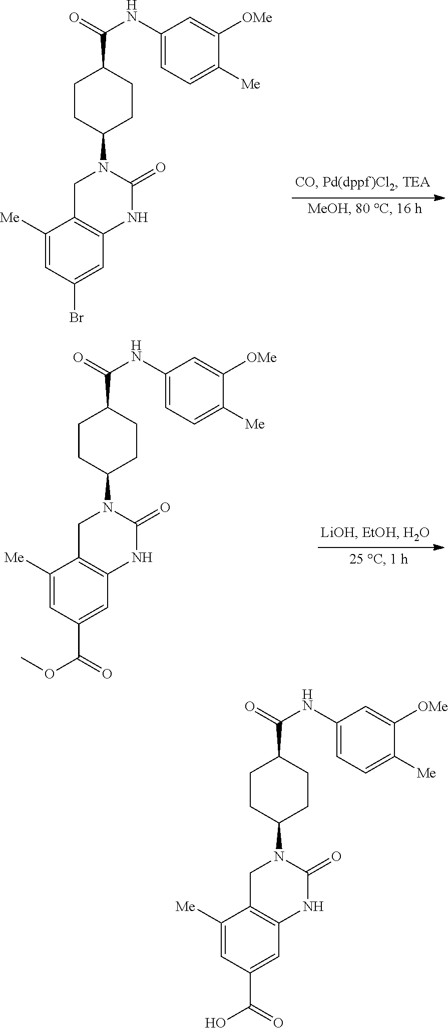

### Step 1

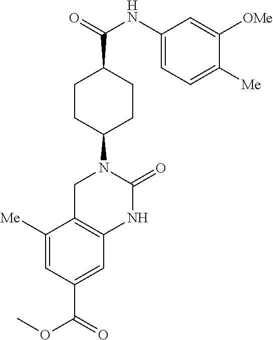 [SMILES: COC(=O)c1cc(C)c2c(c1)NC(=O)N([C@H]1CC[C@@H](C(=O)Nc3ccc(C)c(OC)c3)CC1)C2]

### methyl 3-((1s,4s)-4-(3-methoxy-4-methylphenylcarbamoyl)cyclohexyl)-5-methyl-2-oxo-1,2,3,4-tetrahydroquinazoline-7-carboxylate

A solution of 4-(7-bromo-5-methyl-2-oxo-1,4-dihydroquinazolin-3-yl)-N-(3-methoxy-4-methyl-phenyl)cyclohexanecarboxamide (0.15 g, 310 μmol) and N,N-diethylethanamine (62 mg, 620 μmol, 86 μL) and Pd(dppf)Cl₂ (23 mg, 31 μmol) in Methanol (15 mL) was stirred for 16 hours at 80° C. under carbon monoxide atmosphere (balloon). The mixture was concentrated and the residue was purified by flash chromatography on silica gel (0-50% EtOAc in DCM) to get methyl 3-((1s,4s)-4-(3-methoxy-4-methylphenylcarbamoyl)cyclohexyl)-5-methyl-2-oxo-1,2,3,4-tetrahydroquinazoline-7-carboxylate (0.14 g, 300 μmol, 98% yield) as a white solid (TLC: PE/EtOAc=1/1, R_{f}=0.5). LCMS ESI m/z: 465.9 [M+H]⁺.

### Step 2

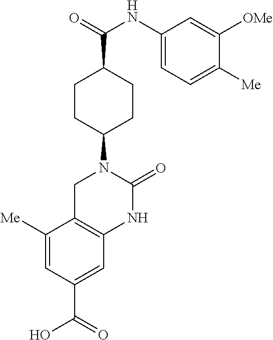 [SMILES: COc1cc(NC(=O)[C@H]2CC[C@@H](N3Cc4c(C)cc(C(=O)O)cc4NC3=O)CC2)ccc1C]

### 3-((1s,4s)-4-(3-methoxy-4-methylphenylcarbamoyl)cyclohexyl)-5-methyl-2-oxo-1,2,3,4-tetrahydroquinazoline-7-carboxylic acid

A solution of methyl 3-((1s,4s)-4-(3-methoxy-4-methylphenylcarbamoyl)cyclohexyl)-5-methyl-2-oxo-1,2,3,4-tetrahydroquinazoline-7-carboxylate (0.12 g, 260 μmol) and LiOH (19 mg, 770 μmol) in Water (0.5 mL)/Ethanol (2 mL) was stirred for 1 hour at 25° C. The mixture was concentrated, extracted with EtOAc (2×50 mL). The aqueous solution was added 6M HCl until the solution reached pH<4. Then the solution was extracted with EtOAc (50 ml×2). The organic layers were dried over Na₂SO₄ and concentrated. The residue was purified by Prep-HPLC to get 3-((1s,4s)-4-(3-methoxy-4-methylphenylcarbamoyl)cyclohexyl)-5-methyl-2-oxo-1,2,3,4-tetrahydroquinazoline-7-carboxylic acid (0.0108 g, 23.9 μmol, 9.28% yield) as a white solid. LCMSESI m/z: 452.2 [M+H]⁺. ¹H NMR (400 MHz, MeOD-d₄) 07.43 (s, 1H), 7.26 (s, 2H), 7.04 (d, J=8.0 Hz, 1H), 6.97 (dd, J=8.0, 2.0 Hz, 1H), 4.43 (s, 2H), 4.40-4.26 (m, 1H), 3.83 (s, 3H), 2.72-2.70 (m, 1H), 2.35-2.07 (m, 10H), 1.88-1.73 (m, 2H), 1.65-1.58 (m, 2H).

### Example 80

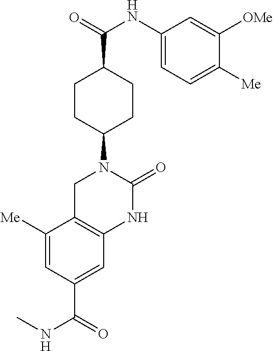 [SMILES: CNC(=O)c1cc(C)c2c(c1)NC(=O)N([C@H]1CC[C@@H](C(=O)Nc3ccc(C)c(OC)c3)CC1)C2]

### 3-((1s,4s)-4-(3-methoxy-4-methylphenylcarbamoyl)cyclohexyl)-N,5-dimethyl-2-oxo-1,2,3,4-tetrahydroquinazoline-7-carboxamide

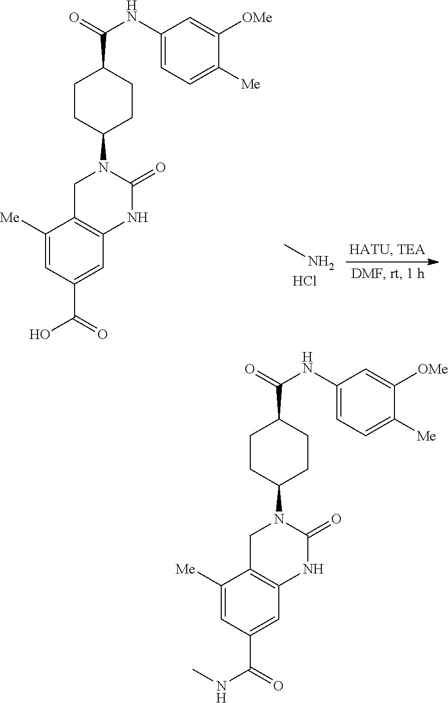

### Step 1

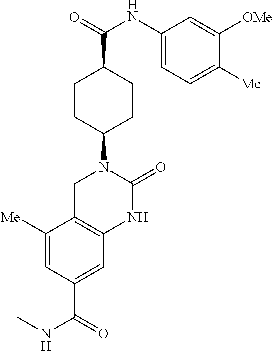 [SMILES: CNC(=O)c1cc(C)c2c(c1)NC(=O)N([C@H]1CC[C@@H](C(=O)Nc3ccc(C)c(OC)c3)CC1)C2]

### 3-((1s,4s)-4-(3-methoxy-4-methylphenylcarbamoyl)cyclohexyl)-N,5-dimethyl-2-oxo-1,2,3,4-tetrahydroquinazoline-7-carboxamide

A solution of methanamine (21 mg, 310 μmol, hydrochloride salt), [dimethylamino(triazolo[4,5-b]pyridin-3-yloxy)methylene]-dimethyl-ammonium; hexafluorophosphate (71 mg, 190 μmol) and N,N-diethylethanamine (47 mg, 470 μmol, 65 μL) in DMF (5 mL) was stirred for 10 minutes. 3-[4-[(3-methoxy-4-methyl-phenyl)carbamoyl]cyclohexyl]-5-methyl-2-oxo-1,4-dihydroquinazoline-7-carboxylic acid (0.070 g, 150 μmol) was added, the mixture was stirred for 1 hour at 25° C. The solution was poured into EtOAc (50 mL) and washed with H₂O (3×50 mL). The combined organic extracts were dried over Na₂SO₄ and evaporated in vacuo. The residue was purified by Prep-HPLC to get 3-((1s,4s)-4-(3-methoxy-4-methylphenylcarbamoyl)cyclohexyl)-N, 5-dimethyl-2-oxo-1,2,3,4-tetrahydroquinazoline-7-carboxamide (14 mg, 31 μmol, 20% yield) as a white solid. LCMS ESI m/z: 465.3 [M+H]⁺. ¹H NMR (400 MHz, DMSO-d₆) δ 9.69 (s, 1H), 9.21 (s, 1H), 8.30-8.20 (m, 1H), 7.29 (d, J=1.6 Hz, 1H), 7.18-7.08 (m, 2H), 7.05-7.00 (m, 2H), 4.26 (s, 2H), 4.25-4.05 (m, 1H), 3.76 (s, 3H), 2.73 (d, J=4.5 Hz, 3H), 2.66-2.64 (m, 1H), 2.20 (s, 3H), 2.15-2.12 (m, 2H), 2.09 (s, 3H), 2.05-1.95 (m, 2H), 1.70-1.60 (m, 2H), 1.50-1.40 (m, 2H).

### Example 81

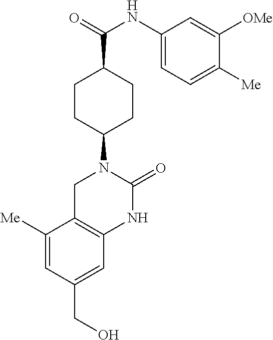 [SMILES: COc1cc(NC(=O)[C@H]2CC[C@@H](N3Cc4c(C)cc(CO)cc4NC3=O)CC2)ccc1C]

### (1s,4s)-4-(7-(hydroxymethyl)-5-methyl-2-oxo-1,2-dihydroquinazolin-3(4H)-yl)-N-(3-methoxy-4-methylphenyl)cyclohexanecarboxamide

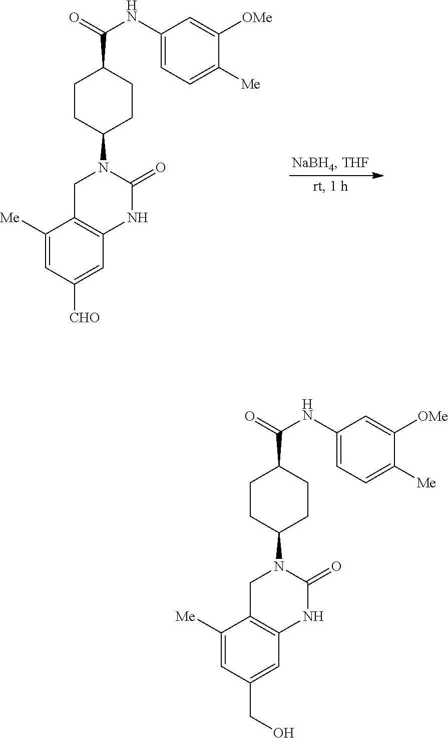 [SMILES: COc1cc(NC(=O)[C@H]2CC[C@@H](N3Cc4c(C)cc(C=O)cc4NC3=O)CC2)ccc1C ; COc1cc(NC(=O)[C@H]2CC[C@@H](N3Cc4c(C)cc(CO)cc4NC3=O)CC2)ccc1C]

### Step 1

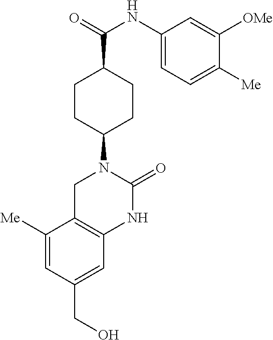 [SMILES: COc1cc(NC(=O)[C@H]2CC[C@@H](N3Cc4c(C)cc(CO)cc4NC3=O)CC2)ccc1C]

### (1s,4s)-4-(7-(hydroxymethyl)-5-methyl-2-oxo-1,2-dihydroquinazolin-3(4H)-yl)-N-(3-methoxy-4-methylphenyl)cyclohexanecarboxamide

To a solution of (1s,4s)-4-(7-formyl-5-methyl-2-oxo-1,2-dihydroquinazolin-3(4H)-yl)-N-(3-methoxy-4-methylphenyl)cyclohexanecarboxamide (0.040 g, 92 μmol) in THF (5 mL) was added sodium borohydride (3.5 mg, 92 μmol). The mixture was stirred for 1 hour at 25° C. The mixture was poured into EtOAc (50 mL) and washed with H₂O (3×50 mL). The combined organic extracts were dried over Na₂SO₄ and evaporated in vacuo. The residue was purified by Prep-HPLC to get (1s,4s)-4-(7-(hydroxymethyl)-5-methyl-2-oxo-1,2-dihydroquinazolin-3(4H)-yl)-N-(3-methoxy-4-methylphenyl)cyclohexanecarboxamide (0.0146 g, 33.3 μmol, 36.3% yield) as a white solid. LCMS ESI m/z: 438.3 [M+H]⁺. ¹H NMR (400 MHz, DMSO-d₆) δ 9.68 (s, 1H), 9.05 (s, 1H), 7.29 (d, J=1.6 Hz, 1H), 7.11 (dd, J=8.0, 2.0 Hz, 1H), 7.02 (d, J=8.0 Hz, 1H), 6.62 (s, 1H), 6.57 (s, 1H), 5.06 (t, J=5.6 Hz, 1H), 4.34 (d, J=5.6 Hz, 2H), 4.25-4.15 (m, 3H), 3.75 (s, 3H), 2.66-2.64 (m, 1H), 2.27-1.82 (m, 10H), 1.76-1.59 (m, 2H), 1.50-1.40 (m, 2H).

### Example 82

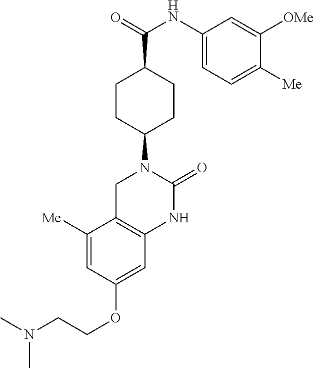 [SMILES: COc1cc(NC(=O)[C@H]2CC[C@@H](N3Cc4c(C)cc(OCCN(C)C)cc4NC3=O)CC2)ccc1C]

### (1s,4s)-4-(7-(2-(dimethylamino)ethoxy)-5-methyl-2-oxo-1,2-dihydroquinazolin-3(4H)-yl)-N-(3-methoxy-4-methylphenyl)cyclohexanecarboxamide

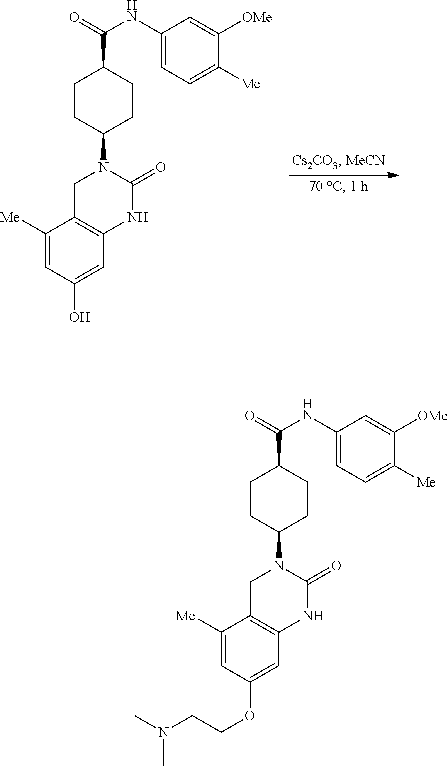 [SMILES: COc1cc(NC(=O)[C@H]2CC[C@@H](N3Cc4c(C)cc(O)cc4NC3=O)CC2)ccc1C ; COc1cc(NC(=O)[C@H]2CC[C@@H](N3Cc4c(C)cc(OCCN(C)C)cc4NC3=O)CC2)ccc1C]

### Step 1

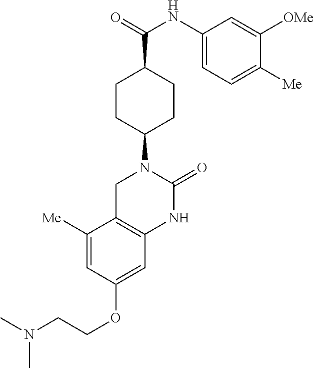 [SMILES: COc1cc(NC(=O)[C@H]2CC[C@@H](N3Cc4c(C)cc(OCCN(C)C)cc4NC3=O)CC2)ccc1C]

### (1s,4s)-4-(7-(2-(dimethylamino)ethoxy)-5-methyl-2-oxo-1,2-dihydroquinazolin-3(4H)-yl)-N-(3-methoxy-4-methylphenyl)cyclohexanecarboxamide

A mixture of (1s,4s)-4-(7-hydroxy-5-methyl-2-oxo-1,2-dihydroquinazolin-3(4H)-yl)-N-(3-methoxy-4-methylphenyl)cyclohexanecarboxamide (Example 77) (30 mg, 71 μmol), 2-chloro-N,N-dimethyl-ethanamine (10 mg, 71 μmol, hydrochloride salt) and Cs₂CO₃ (69 mg, 210 μmol) in MeCN (1 mL) was stirred at 70° C. for 16 hours. The reaction mixture was filtered and concentrated to the crude product. The residue was purified by Prep-HPLC to give (1s,4s)-4-(7-(2-(dimethylamino)ethoxy)-5-methyl-2-oxo-1,2-dihydroquinazolin-3(4H)-yl)-N-(3-methoxy-4-methylphenyl)cyclohexanecarboxamide (14 mg, 28 μmol, 40% yield) as a light-yellow solid. LCMS (ESI) m/z: 494.9 [M+H]⁺. ¹H NMR (400 MHz, DMSO-d₆) δ 9.68 (s, 1H), 8.98 (s, 1H), 7.29 (d, J=1.7 Hz, 1H), 7.11 (dd, J=8.0, 1.8 Hz, 1H), 7.02 (d, J=8.1 Hz, 1H), 6.32 (d, J=2.2 Hz, 1H), 6.18 (d, J=2.2 Hz, 1H), 4.23-4.12 (m, 3H), 3.93 (t, J=5.8 Hz, 2H), 3.75 (s, 3H), 2.66-2.64 (m, 1H), 2.59 (t, J=5.8 Hz, 2H), 2.22 (s, 6H), 2.15-2.08 (m, 8H), 2.04-1.98 (m, 2H), 1.68-1.65 (m, 2H), 1.46-1.43 (m, 2H).

### Example 83

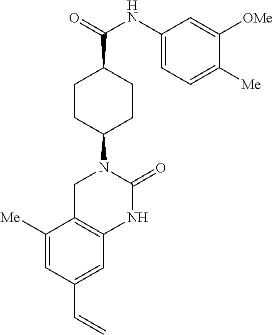 [SMILES: C=Cc1cc(C)c2c(c1)NC(=O)N([C@H]1CC[C@@H](C(=O)Nc3ccc(C)c(OC)c3)CC1)C2]

### (1s,4s)-N-(3-methoxy-4-methylphenyl)-4-(5-methyl-2-oxo-7-vinyl-1,2-dihydroquinazolin-3(4H)-yl)cyclohexanecarboxamide

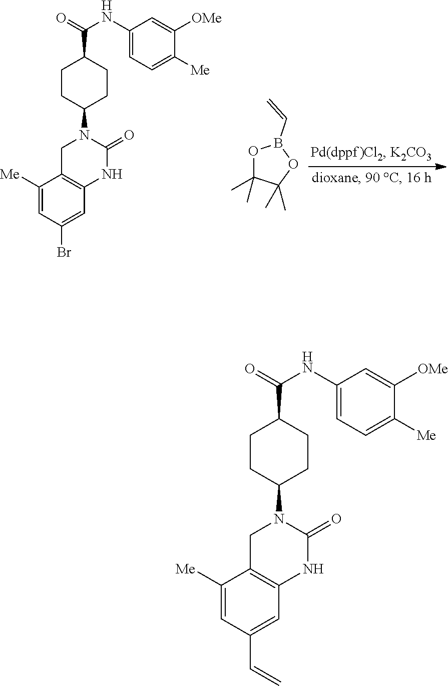 [SMILES: COc1cc(NC(=O)[C@H]2CC[C@@H](N3Cc4c(C)cc(Br)cc4NC3=O)CC2)ccc1C ; C=CB1OC(C)(C)C(C)(C)O1 ; C=Cc1cc(C)c2c(c1)NC(=O)N([C@H]1CC[C@@H](C(=O)Nc3ccc(C)c(OC)c3)CC1)C2]

### Step 1

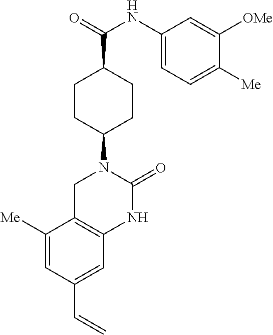 [SMILES: C=Cc1cc(C)c2c(c1)NC(=O)N([C@H]1CC[C@@H](C(=O)Nc3ccc(C)c(OC)c3)CC1)C2]

### (1s,4s)-N-(3-methoxy-4-methylphenyl)-4-(5-methyl-2-oxo-7-vinyl-1,2-dihydroquinazolin-3(4H)-yl)cyclohexanecarboxamide

A solution of 4-(7-bromo-5-methyl-2-oxo-1,4-dihydroquinazolin-3-yl)-N-(3-methoxy-4-methyl-phenyl)cyclohexanecarboxamide (0.050 g, 100 μmol), 4,4,5,5-tetramethyl-2-vinyl-1,3,2-dioxaborolane (32 mg, 210 μmol), tripotassium carbonate (43 mg, 310 μmol) and Pd(dppf)Cl₂ (7.5 mg, 10 μmol) in dioxane (3 mL) was stirred for 16 hours at 90° C. under N₂. The mixture was poured into EtOAc (50 mL) and washed with H₂O (3×50 mL). The combined organic extracts were dried over Na₂SO₄ and evaporated in vacuo. The residue was purified by Prep-HPLC to get (1s,4s)-N-(3-methoxy-4-methylphenyl)-4-(5-methyl-2-oxo-7-vinyl-1,2-dihydroquinazolin-3(4H)-yl)cyclohexanecarboxamide (0.0108 g, 24.0 μmol, 23.4% yield) as a white solid. LCMS (ESI) m/z: 434.3 [M+H]⁺. ¹H NMR (400 MHz, MeOD-d₄) δ 7.25 (d, J=1.6 Hz, 1H), 7.03 (d, J=8.0 Hz, 1H), 6.96 (dd, J=8.0, 2.0 Hz, 1H), 6.83 (s, 1H), 6.68 (s, 1H), 6.65-6.55 (m, 1H), 5.69 (d, J=17.6 Hz, 1H), 5.17 (d, J=11.2 Hz, 1H), 4.40-4.25 (m, 3H), 3.83 (s, 3H), 2.72-2.70 (m, 1H), 2.32-2.05 (m, 10H), 1.85-1.75 (m, 2H), 1.65-1.55 (m, 2H).

### Example 84

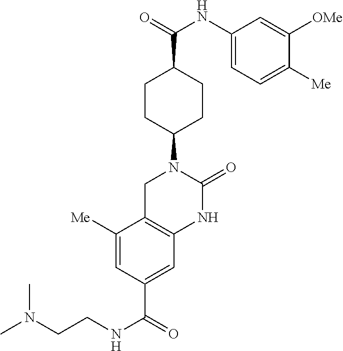 [SMILES: COc1cc(NC(=O)[C@H]2CC[C@@H](N3Cc4c(C)cc(C(=O)NCCN(C)C)cc4NC3=O)CC2)ccc1C]

### N-(2-(dimethylamino)ethyl)-3-((1s,4s)-4-(3-methoxy-4-methylphenylcarbamoyl)cyclohexyl)-5-methyl-2-oxo-1,2,3,4-tetrahydroquinazoline-7-carboxamide

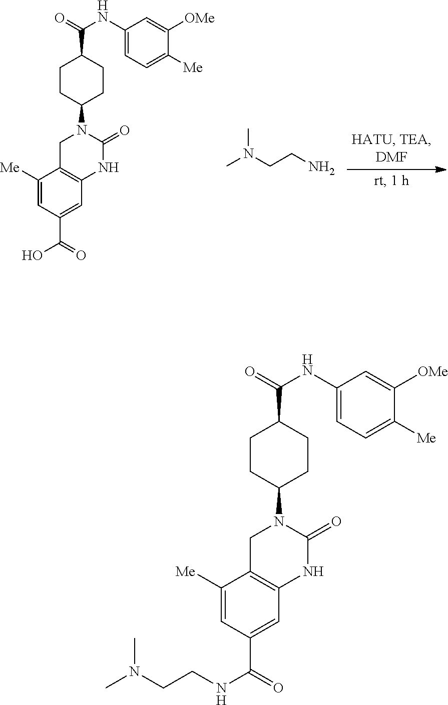

### Step 1

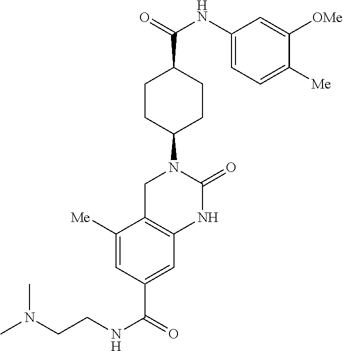 [SMILES: COc1cc(NC(=O)[C@H]2CC[C@@H](N3Cc4c(C)cc(C(=O)NCCN(C)C)cc4NC3=O)CC2)ccc1C]

### N-(2-(dimethylamino)ethyl)-3-((1s,4s)-4-(3-methoxy-4-methylphenylcarbamoyl)cyclohexyl)-5-methyl-2-oxo-1,2,3,4-tetrahydroquinazoline-7-carboxamide

A solution of N′,N′-dimethylethane-1,2-diamine (9.8 mg, 110 μmol, 12 μL), [dimethylamino (triazolo[4,5-b]pyridin-3-yloxy)methylene]-dimethyl-ammonium; hexafluorophosphate (51 mg, 130 μmol) and N,N-diethylethanamine (34 mg, 330 μmol, 46 μL), 3-[4-[(3-methoxy-4-methyl-phenyl)carbamoyl]cyclohexyl]-5-methyl-2-oxo-1,4-dihydroquinazoline-7-carboxylic acid (Example 79) (0.050 g, 110 μmol) in DMF (2 mL) was stirred for 1 hour at 25° C. The solution was poured into EtOAc (50 mL) and washed with H₂O (3×50 mL). The combined organic extracts were dried over Na₂SO₄ and evaporated in vacuo. The residue was purified by Prep-HPLC to get N-(2-(dimethylamino)ethyl)-3-((1s,4s)-4-(3-methoxy-4-methylphenylcarbamoyl)cyclohexyl)-5-methyl-2-oxo-1,2,3,4-tetrahydroquinazoline-7-carboxamide (0.014 g, 27 μmol, 24% yield) as a white solid. LCMS (ESI) m/z: 521.9 [M+H]⁺. ¹H NMR (400 MHz, MeOD-d₄) δ 8.48 (s, 1H), 7.8-7.23 (m, 2H), 7.08 (s, 1H), 7.04 (d, J=8.4 Hz, 1H), 6.98 (dd, J=8.0, 2.0 Hz, 1H), 4.43 (s, 2H), 4.40-4.30 (m, 1H), 3.84 (s, 3H), 3.64 (t, J=6.0 Hz, 2H), 3.10-3.06 (m, 2H), 2.73-2.71 (m, 7H), 2.31 (s, 3H), 2.26-2.08 (m, 7H), 1.85-1.75 (m, 2H), 1.65-1.55 (m, 2H).

### Example 85

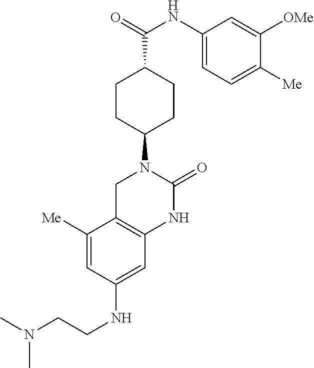 [SMILES: COc1cc(NC(=O)[C@H]2CC[C@H](N3Cc4c(C)cc(NCCN(C)C)cc4NC3=O)CC2)ccc1C]

### (1r,4r)-4-(7-(2-(dimethylamino)ethylamino)-5-methyl-2-oxo-1,2-dihydroquinazolin-3(4H)-yl)-N-(3-methoxy-4-methylphenyl)cyclohexanecarboxamide

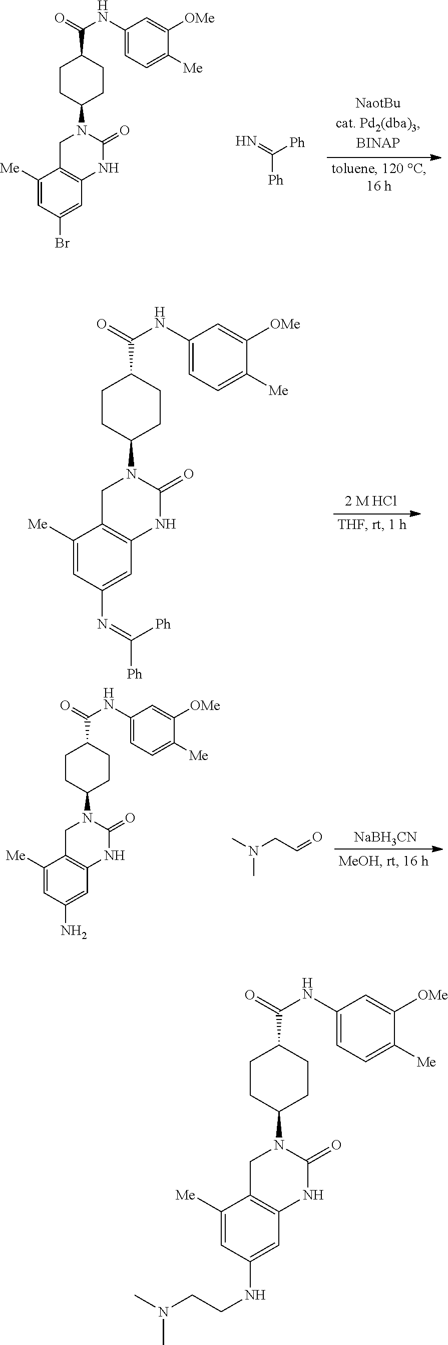

### Step 1

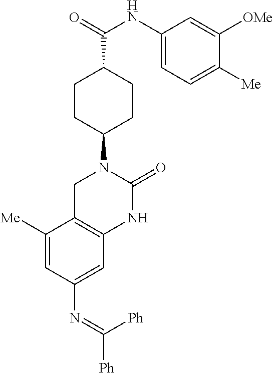 [SMILES: COc1cc(NC(=O)[C@H]2CC[C@H](N3Cc4c(C)cc(N=C(c5ccccc5)c5ccccc5)cc4NC3=O)CC2)ccc1C]

### (1r,4r)-4-(7-(diphenylmethyleneamino)-5-methyl-2-oxo-1,2-dihydroquinazolin-3(4H)-yl)-N-(3-methoxy-4-methylphenyl)cyclohexanecarboxamide

A mixture of diphenylmethanimine (559 mg, 3.08 mmol), benzyl-[1-[2-[benzyl(phenyl)phosphanyl]-1-naphthyl]-2-naphthyl]-phenyl-phosphane (40 mg, 62 μmol), 4-(7-bromo-5-methyl-2-oxo-1,4-dihydroquinazolin-3-yl)-N-(3-methoxy-4-methyl-phenyl)cyclohexanecarboxamide (Example 74) (0.30 g, 620 μmol), NaOtBu (119 mg, 1.23 mmol) and Pd₂(dba)₃ (56 mg, 62 μmol) in toluene (10 mL) was stirred for 16 hours at 120° C. The mixture was poured into EtOAc (50 mL) and washed with H₂O (3×50 mL). The combined organic extracts were dried over Na₂SO₄ and evaporated in vacuo. The residue was purified by flash chromatography on silica gel (0-5% MeOH in DCM) to get (1r,4r)-4-(7-(diphenylmethyleneamino)-5-methyl-2-oxo-1,2-dihydroquinazolin-3(4H)-yl)-N-(3-methoxy-4-methylphenyl)cyclohexanecarboxamide (0.30 g, 510 μmol, 83% yield) as a white solid (TLC: DCM/MeOH=20/1, R_{f}=0.4). LCMS (ESI) m/z: 586.8 [M+H]⁺.

### Step 2

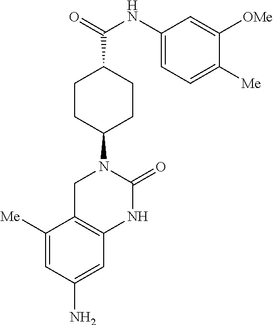 [SMILES: COc1cc(NC(=O)[C@H]2CC[C@H](N3Cc4c(C)cc(N)cc4NC3=O)CC2)ccc1C]

### (1r,4r)-4-(7-amino-5-methyl-2-oxo-1,2-dihydroquinazolin-3(4H)-yl)-N-(3-methoxy-4-methylphenyl)cyclohexanecarboxamide

To a solution of (1r,4r)-4-(7-(diphenylmethyleneamino)-5-methyl-2-oxo-1,2-dihydroquinazolin-3(4H)-yl)-N-(3-methoxy-4-methylphenyl)cyclohexanecarboxamide (0.30 g, 510 μmol) in THF (10 mL) was added 2M HCl (3 mL). The mixture was concentrated and recrystallized from EtOAc (10 mL) to get (1r,4r)-4-(7-amino-5-methyl-2-oxo-1,2-dihydroquinazolin-3(4H)-yl)-N-(3-methoxy-4-methylphenyl)cyclohexanecarboxamide (0.20 g, 470 μmol, 93% yield) as a yellow solid. LCMS (ESI) m/z: 422.9 [M+H]⁺.

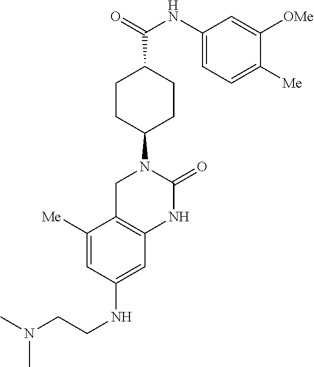 [SMILES: COc1cc(NC(=O)[C@H]2CC[C@H](N3Cc4c(C)cc(NCCN(C)C)cc4NC3=O)CC2)ccc1C]

### (1r,4r)-4-(7-(2-(dimethylamino)ethylamino)-5-methyl-2-oxo-1,2-dihydroquinazolin-3(4H)-yl)-N-(3-methoxy-4-methylphenyl)cyclohexanecarboxamide

To a stirred solution of (1r,4r)-4-(7-amino-5-methyl-2-oxo-1,2-dihydroquinazolin-3(4H)-yl)-N-(3-methoxy-4-methylphenyl)cyclohexanecarboxamide (0.20 g, 470 μmol) and 2-(dimethylamino)acetaldehyde (82 mg, 950 μmol) at 25° C. in MeOH (10 mL) was added sodium cyanoborohydride (89 mg, 1.4 mmol) for 16 hour. The reaction mixture was poured into EtOAc (50 mL) and washed with H₂O (3×50 mL). The combined organic extracts were dried over Na₂SO₄ and evaporated in vacuo. The residue was purified by Prep-HPLC to get (1r,4r)-4-(7-(2-(dimethylamino)ethylamino)-5-methyl-2-oxo-1,2-dihydroquinazolin-3(4H)-yl)-N-(3-methoxy-4-methylphenyl)cyclohexanecarboxamide (0.0148 g, 29.9 μmol, 6.31% yield) as a white solid. LCMS ESI m/z: 493.9 [M+H]⁺. ¹H NMR (400 MHz, DMSO-d₆) δ 9.74 (s, 1H), 8.80 (s, 1H), 7.36 (s, 1H), 7.05-6.98 (m, 2H), 5.99 (s, 1H), 5.85 (s, 1H), 5.18-5.14 (m, 1H), 4.20-4.05 (m, 3H), 3.74 (s, 3H), 3.05-2.95 (m, 2H), 2.45-2.35 (m, 2H), 2.34-2.28 (m, 1H), 2.16 (s, 6H), 2.10-1.05 (m, 5H), 2.00-1.87 (m, 2H), 1.70-1.65 (m, 2H), 1.64-1.50 (m, 2H).

### Example 86

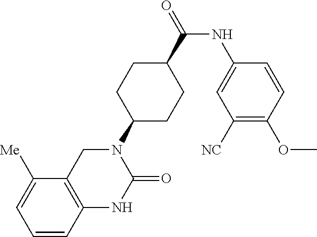 [SMILES: COc1ccc(NC(=O)[C@H]2CC[C@@H](N3Cc4c(C)cccc4NC3=O)CC2)cc1C#N]

### (1s,4s)-N-(3-cyano-4-methoxyphenyl)-4-(5-methyl-2-oxo-1,2-dihydroquinazolin-3(4H)-yl)cyclohexanecarboxamide

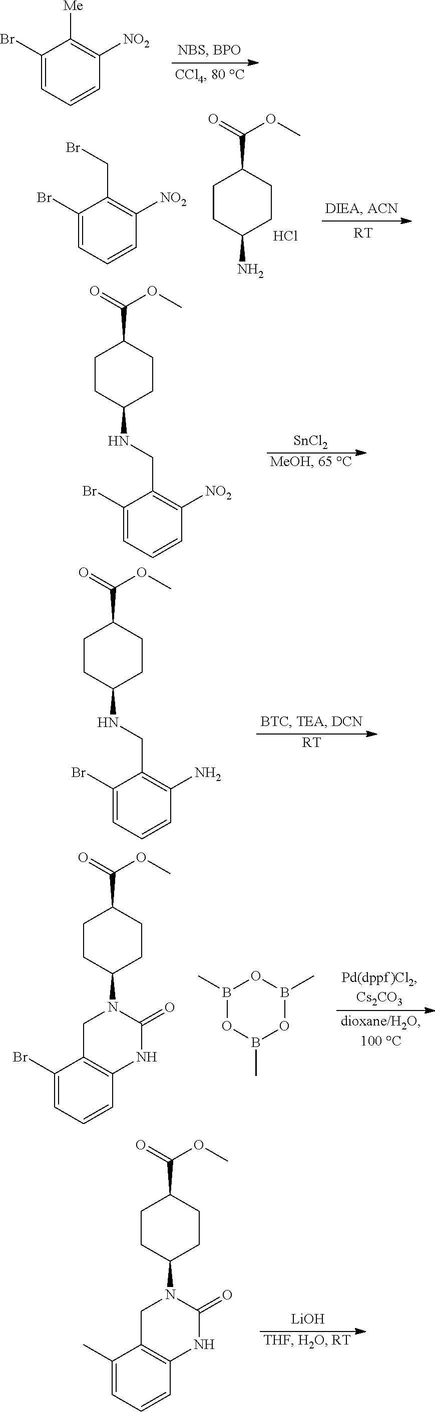

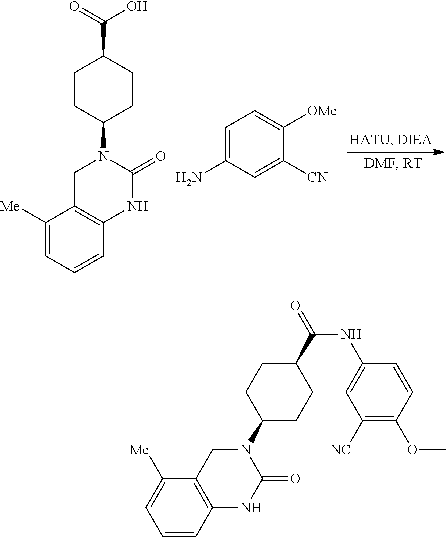

### Step 1

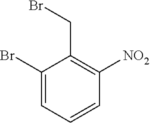 [SMILES: O=[N+]([O-])c1cccc(Br)c1CBr]

### 1-bromo-2-(bromomethyl)-3-nitrobenzene

To a solution of 1-bromo-2-methyl-3-nitro-benzene (15 g, 69 mmol) in CCl₄ (150 mL) was added benzoyl benzenecarboperoxoate (1.68 g, 6.94 mmol) and NBS (13.6 g, 76.4 mmol). The mixture was stirred at 80° C. overnight. The solution was poured into water (50 mL) and extracted with EtOAc (50 mL*3). The organic layers were washed with brine, dried by Na₂SO₄ and concentrated. The residue was purified by silica gel chromatography to give 1-bromo-2-(bromomethyl)-3-nitro-benzene (18 g, 61 mmol, 88% yield) as a light-yellow solid. ¹H NMR (400 MHz, CDCl₃) δ 7.90-7.85 (m, 2H), 7.35 (t, J=8.1 Hz, 1H), 4.89 (s, 2H).

### Step 2

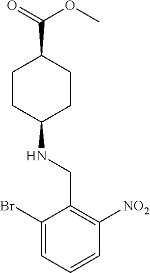 [SMILES: COC(=O)[C@H]1CC[C@@H](NCc2c(Br)cccc2[N+](=O)[O-])CC1]

### (1s,4s)-methyl 4-(2-bromo-6-nitrobenzylamino)cyclohexanecarboxylate

To a solution of 1-bromo-2-(bromomethyl)-3-nitro-benzene (5.00 g, 17.0 mmol), methyl 4-aminocyclohexanecarboxylate (4.00 g, 25.4 mmol) in ACN (49 mL) was added DIPEA (8.76 g, 67.8 mmol, 11.8 mL). The resulting mixture was stirred at room temperature overnight. The solution was poured into water and extracted with EtOAc. The organic layers were washed with brine, dried by Na₂SO₄ and concentrated. The residue was purified by silica gel chromatography to give (1s,4s)-methyl 4-(2-bromo-6-nitrobenzylamino)cyclohexanecarboxylate (5.00 g, 13.5 mmol, 79.5% yield) as yellow oil. LCMS (ESI) m/z: 370.7 [M+H]⁺.

### Step 3

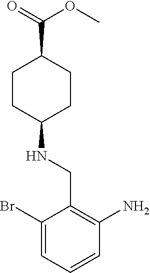 [SMILES: COC(=O)[C@H]1CC[C@@H](NCc2c(N)cccc2Br)CC1]

### (1s,4s)-methyl 4-(2-amino-6-bromobenzylamino)cyclohexanecarboxylate

A solution of (1s,4s)-methyl 4-(2-bromo-6-nitrobenzylamino)cyclohexanecarboxylate (3.4 g, 9.2 mmol) and tin (II) chloride dihydrate (5.63 g, 45.8 mmol) in MeOH (20 mL) was stirred at 65° C. overnight. The reaction mixture was cooled to RT and adjusted pH to 8 by NaHCO₃. The solution was filtered through a celite pad, and the filtrate was extracted by EtOAc. The organic layers were washed by brine, dried and concentrated. The residue was purified by silica gel chromatography to give (1s,4s)-methyl 4-(2-amino-6-bromobenzylamino)cyclohexanecarboxylate (2.40 g, 7.03 mmol, 76.8% yield) as yellow oil. LCMS (ESI) m/z: 343.1 [M+H]⁺.

### Step 4

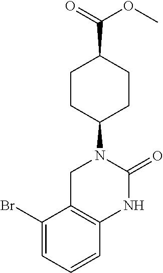 [SMILES: COC(=O)[C@H]1CC[C@@H](N2Cc3c(Br)cccc3NC2=O)CC1]

### (1s,4s)-methyl 4-(5-bromo-2-oxo-1,2-dihydroquinazolin-3(4H)-yl)cyclohexanecarboxylate

To a solution of (1s,4s)-methyl 4-(2-amino-6-bromobenzylamino)cyclohexanecarboxylate (1.50 g, 4.40 mmol) and BTC (2.6 g, 8.8 mmol) in DCM (10 mL) was added TEA (1.33 g, 13.2 mmol, 1.84 mL). The resulting mixture was stirred at 25° C. for 2 hours. The reaction mixture was poured into water and extracted with DCM. The organic layers were washed with brine, dried by Na₂SO₄, and concentrated to give (1s,4s)-methyl 4-(5-bromo-2-oxo-1,2-dihydroquinazolin-3(4H)-yl)cyclohexanecarboxylate (1.2 g, 3.3 mmol, 74% yield) as a white solid. LCMS (ESI) m/z: 369.1 [M+H]⁺.

### Step 5

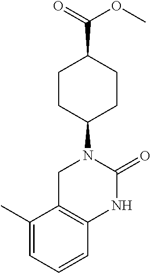

### (1s,4s)-methyl 4-(5-methyl-2-oxo-1,2-dihydroquinazolin-3(4H)-yl)cyclohexanecarboxylate

To a solution of (1s,4s)-methyl 4-(5-bromo-2-oxo-1,2-dihydroquinazolin-3(4H)-yl)cyclohexanecarboxylate (1.46 g, 3.98 mmol) and 2,4,6-trimethyl-1,3,5,2,4,6-trioxatriborinane (998 mg, 7.95 mmol) in dioxane (15 mL) and H₂O (5 mL) was added Pd(dppf)Cl₂ (291 mg, 398 μmol) and Cs₂CO₃ (3.89 g, 11.9 mmol). The mixture was stirred at 100° C. overnight under N₂. The reaction mixture was poured into water and extracted with EtOAc. The organic phases were washed with brine, dried by Na₂SO₄. The residue was purified by silica gel chromatography to give (1s,4s)-methyl 4-(5-methyl-2-oxo-1,2-dihydroquinazolin-3(4H)-yl)cyclohexanecarboxylate (900 mg, 2.98 mmol, 74.9% yield) as white solid. LCMS (ESI) m/z: 303.2 [M+H]⁺

### Step 6

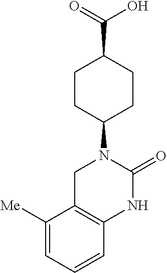 [SMILES: Cc1cccc2c1CN([C@H]1CC[C@@H](C(=O)O)CC1)C(=O)N2]

### (1s,4s)-4-(5-methyl-2-oxo-1,2-dihydroquinazolin-3(4H)-yl)cyclohexanecarboxylic Acid

To a solution of (1s,4s)-methyl 4-(5-methyl-2-oxo-1,2-dihydroquinazolin-3(4H)-yl)cyclohexanecarboxylate (1.2 g, 4.0 mmol) in H₂O (5 mL) and THF (5 mL) was added LiOH (285 mg, 11.9 mmol). The mixture was stirred at rt overnight. The solution was added dropwise 1M HCl until pH reached 5-6. The solids were collected and dried to give (1s,4s)-4-(5-methyl-2-oxo-1,2-dihydroquinazolin-3(4H)-yl)cyclohexanecarboxylic acid (700 mg, 2.4 mmol, 61% yield) as a white solid. LCMS (ESI) m/z: 289.2 [M+H]⁺. ¹H NMR (400 MHz, DMSO-d₆) δ 12.26 (b, 1H), 9.05 (s, 1H), 6.99 (t, J=7.7 Hz, 1H), 6.69 (d, J=7.5 Hz, 1H), 6.58 (d, J=7.9 Hz, 1H), 4.19 (s, 2H), 4.17-4.11 (m, 1H), 2.60-2.58 (m, 1H), 2.16-2.14 (m, 5H), 1.78-1.6 (m, 2H), 1.46-1.45 (m, 4H).

### Step 7

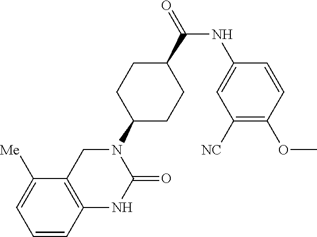 [SMILES: COc1ccc(NC(=O)[C@H]2CC[C@@H](N3Cc4c(C)cccc4NC3=O)CC2)cc1C#N]

### (1s,4s)-N-(3-cyano-4-methoxyphenyl)-4-(5-methyl-2-oxo-1,2-dihydroquinazolin-3(4H)-yl)cyclohexanecarboxamide

To a solution of (1s,4s)-4-(5-methyl-2-oxo-1,2-dihydroquinazolin-3(4H)-yl)cyclohexanecarboxylic acid (50 mg, 170 μmol), HATU (79 mg, 210 μmol), DIPEA (27 mg, 210 μmol) in DMF (3 mL) was added 5-amino-2-methoxy-benzonitrile (28 mg, 190 μmol). The solution was filtered and purified by Prep-HPLC to give (1s,4s)-N-(3-cyano-4-methoxyphenyl)-4-(5-methyl-2-oxo-1,2-dihydroquinazolin-3(4H)-yl)cyclohexanecarboxamide (32 mg, 76 μmol, 44% yield) as white solid. LCMS (ESI) m/z: 418.9 [M+H]⁺. ¹H NMR (400 MHz, DMSO-d₆) δ 9.91 (s, 1H), 9.04 (s, 1H), 7.98 (d, J=2.6 Hz, 1H), 7.77 (dd, J=9.2, 2.6 Hz, 1H), 7.21 (d, J=9.2 Hz, 1H), 6.97 (t, J=7.7 Hz, 1H), 6.67 (d, J=7.5 Hz, 1H), 6.57 (d, J=7.8 Hz, 1H), 4.23-4.14 (m, 3H), 3.87 (s, 3H), 2.65-2.63 (m, 1H), 2.15-2.10 (m, 5H), 2.04-1.92 (m, 2H), 1.69-1.63 (m, 2H), 1.46-1.43 (m, 2H).

### Example 87

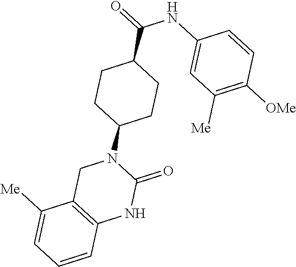 [SMILES: COc1ccc(NC(=O)[C@H]2CC[C@@H](N3Cc4c(C)cccc4NC3=O)CC2)cc1C]

### (1s,4s)-N-(4-methoxy-3-methylphenyl)-4-(5-methyl-2-oxo-1,2-dihydroquinazolin-3(4H)-yl)cyclohexanecarboxamide

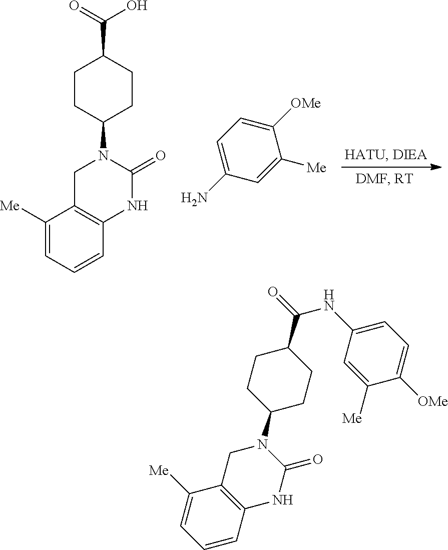 [SMILES: Cc1cccc2c1CN([C@H]1CC[C@@H](C(=O)O)CC1)C(=O)N2 ; COc1ccc(N)cc1C ; COc1ccc(NC(=O)[C@H]2CC[C@@H](N3Cc4c(C)cccc4NC3=O)CC2)cc1C]

### Step 1

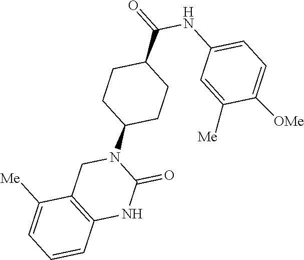 [SMILES: COc1ccc(NC(=O)[C@H]2CC[C@@H](N3Cc4c(C)cccc4NC3=O)CC2)cc1C]

### (1s,4s)-N-(4-methoxy-3-methylphenyl)-4-(5-methyl-2-oxo-1,2-dihydroquinazolin-3(4H)-yl)cyclohexanecarboxamide

Following the synthetic procedure in Example 86, but starting with 4-methoxy-3-methylaniline gave the title compound as a white solid. LCMS (ESI) m/z: 407.9 [M+H]⁺. ¹H NMR (400 MHz, DMSO-d₆) δ 9.52 (s, 1H), 9.03 (s, 1H), 7.36 (d, J=2.3 Hz, 2H), 6.97 (t, J=7.7 Hz, 1H), 6.87-6.81 (m, 1H), 6.68 (d, J=7.5 Hz, 1H), 6.57 (d, J=7.8 Hz, 1H), 4.22-4.19 (m, 3H), 3.73 (s, 3H), 2.62-2.60 (m, 1H), 2.16-1.95 (m, 10H), 1.67-1.63 (m, 2H), 1.46-1.42 (m, 2H).

### Example 88

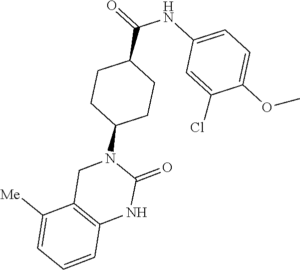 [SMILES: COc1ccc(NC(=O)[C@H]2CC[C@@H](N3Cc4c(C)cccc4NC3=O)CC2)cc1Cl]

### (1s,4s)-N-(3-chloro-4-methoxyphenyl)-4-(5-methyl-2-oxo-1,2-dihydroquinazolin-3(4H)-yl)cyclohexanecarboxamide

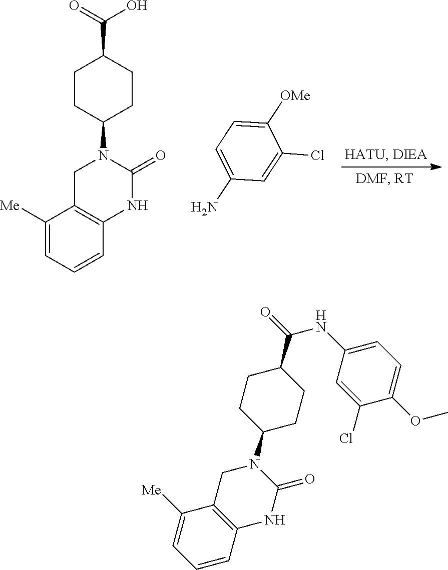 [SMILES: Cc1cccc2c1CN([C@H]1CC[C@@H](C(=O)O)CC1)C(=O)N2 ; COc1ccc(N)cc1Cl ; COc1ccc(NC(=O)[C@H]2CC[C@@H](N3Cc4c(C)cccc4NC3=O)CC2)cc1Cl]

### Step 1

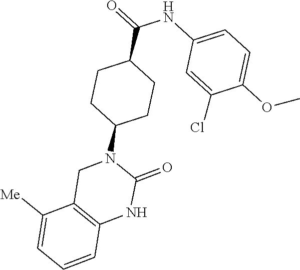 [SMILES: COc1ccc(NC(=O)[C@H]2CC[C@@H](N3Cc4c(C)cccc4NC3=O)CC2)cc1Cl]

### (1s,4s)-N-(3-chloro-4-methoxyphenyl)-4-(5-methyl-2-oxo-1,2-dihydroquinazolin-3(4H)-yl)cyclohexanecarboxamide

Following the synthetic procedure in Example 86, but starting with 3-chloro-4-methoxyaniline gave the title compound as a white solid. LCMS (ESI) m/z: 427.9 [M+H]⁺. ¹H NMR (400 MHz, DMSO-d₆) δ 9.76 (s, 1H), 9.03 (s, 1H), 7.79 (d, J=2.5 Hz, 1H), 7.45 (dd, J=8.9, 2.5 Hz, 1H), 7.09 (d, J=9.0 Hz, 1H), 6.97 (t, J=7.7 Hz, 1H), 6.67 (d, J=7.5 Hz, 1H), 6.57 (d, J=8.0 Hz, 1H), 4.25-4.13 (m, 3H), 3.80 (s, 3H), 2.64-2.60 (m, 1H), 2.17-1.92 (m, 7H), 1.68-1.62 (m, 2H), 1.47-1.42 (m, 2H).

### Example 89

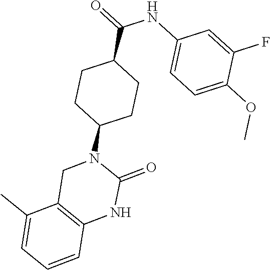 [SMILES: COc1ccc(NC(=O)[C@H]2CC[C@@H](N3Cc4c(C)cccc4NC3=O)CC2)cc1F]

### (1s,4s)-N-(3-fluoro-4-methoxyphenyl)-4-(5-methyl-2-oxo-1,2-dihydroquinazolin-3(4H)-yl)cyclohexanecarboxamide

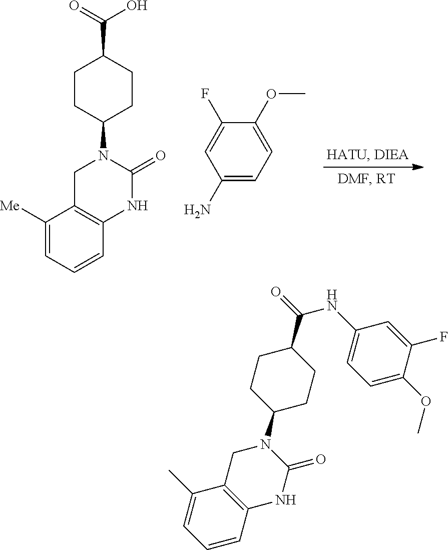

### Step 1

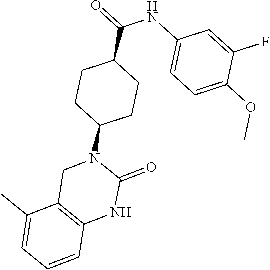 [SMILES: COc1ccc(NC(=O)[C@H]2CC[C@@H](N3Cc4c(C)cccc4NC3=O)CC2)cc1F]

### (1s,4s)-N-(3-fluoro-4-methoxyphenyl)-4-(5-methyl-2-oxo-1,2-dihydroquinazolin-3(4H)-yl)cyclohexanecarboxamide

Following the synthetic procedure in Example 86, but starting with 3-fluoro-4-methoxyaniline gave the title compound as a white solid. LCMS (ESI) m/z: 412.2 [M+H]⁺. ¹H NMR (400 MHz, DMSO-d₆) δ 9.79 (s, 1H), 9.05 (s, 1H), 7.61 (dd, J=13.9, 2.4 Hz, 1H), 7.32-7.25 (m, 1H), 7.10 (t, J=9.3 Hz, 1H), 6.99 (t, J=7.7 Hz, 1H), 6.69 (d, J=7.5 Hz, 1H), 6.58 (d, J=7.8 Hz, 1H), 4.22 (s, 2H), 4.21-4.15 (m, 1H), 3.80 (s, 3H), 2.65-2.63 (m, 1H), 2.15 (s, 3H), 2.13-2.10 (m, 2H), 2.04-1.99 (m, 2H), 1.72-1.59 (m, 2H), 1.44-1.44 (m, 2H).

### Example 90

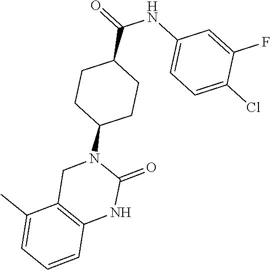 [SMILES: Cc1cccc2c1CN([C@H]1CC[C@@H](C(=O)Nc3ccc(Cl)c(F)c3)CC1)C(=O)N2]

### (1s,4s)-N-(4-chloro-3-fluorophenyl)-4-(5-methyl-2-oxo-1,2-dihydroquinazolin-3(4H)-yl)cyclohexanecarboxamide

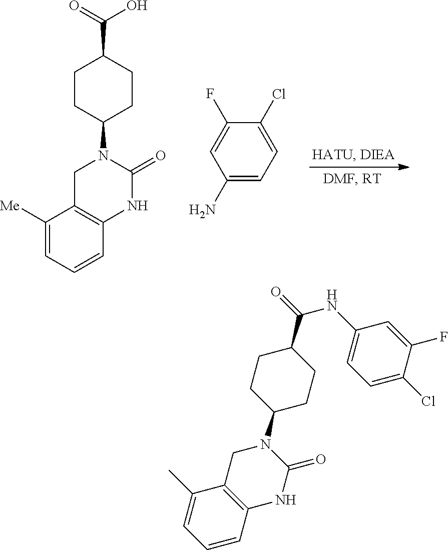 [SMILES: Cc1cccc2c1CN([C@H]1CC[C@@H](C(=O)O)CC1)C(=O)N2 ; Nc1ccc(Cl)c(F)c1 ; Cc1cccc2c1CN([C@H]1CC[C@@H](C(=O)Nc3ccc(Cl)c(F)c3)CC1)C(=O)N2]

### Step 1

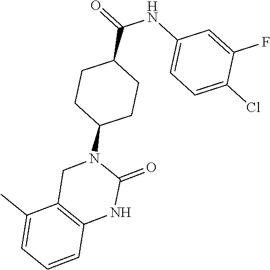 [SMILES: Cc1cccc2c1CN([C@H]1CC[C@@H](C(=O)Nc3ccc(Cl)c(F)c3)CC1)C(=O)N2]

### (1s,4s)-N-(4-chloro-3-fluorophenyl)-4-(5-methyl-2-oxo-1,2-dihydroquinazolin-3(4H)-yl)cyclohexanecarboxamide

Following the synthetic procedure in Example 86, but starting with 4-chloro-3-fluoroaniline gave the title compound as a white solid. LCMS (ESI) m/z: 416.2 [M+H]⁺. ¹H NMR (400 MHz, DMSO-d₆) δ 10.12 (s, 1H), 9.05 (s, 1H), 7.83 (dd, J=12.1, 2.3 Hz, 1H), 7.50 (t, J=8.7 Hz, 1H), 7.42-7.34 (m, 1H), 6.99 (t, J=7.7 Hz, 1H), 6.69 (d, J=7.5 Hz, 1H), 6.58 (d, J=7.8 Hz, 1H), 4.28-4.12 (m, 3H), 2.69-2.66 (m, 1H), 2.17-2.07 (m, 5H), 2.04-1.93 (m, 2H), 1.71-1.66 (m, 2H), 1.48-1.45 (m, 2H).

### Example 91

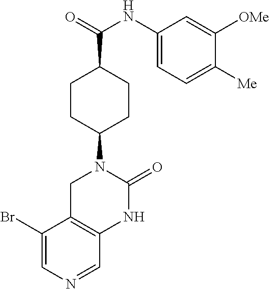 [SMILES: COc1cc(NC(=O)[C@H]2CC[C@@H](N3Cc4c(Br)cncc4NC3=O)CC2)ccc1C]

### (1s,4s)-4-(5-bromo-2-oxo-1,2-dihydropyrido[3,4-d]pyrimidin-3(4H)-yl)-N-(3-methoxy-4-methylphenyl)cyclohexanecarboxamide

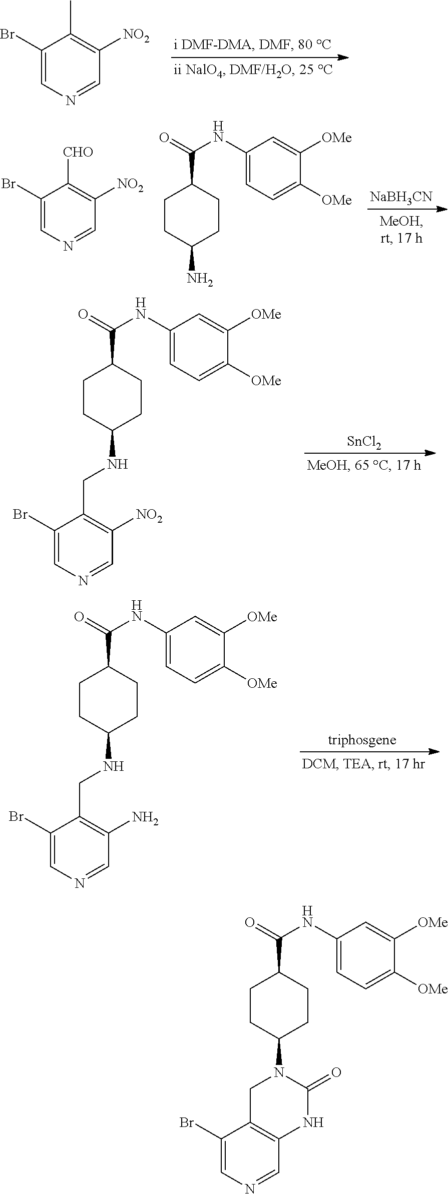

### Step 1

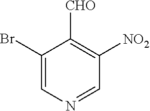 [SMILES: O=Cc1c(Br)cncc1[N+](=O)[O-]]

### 3-bromo-5-nitroisonicotinaldehyde

To a solution of 3-bromo-4-methyl-5-nitro-pyridine (10 g, 46 mmol) in DMF (50 mL) was added DMF-DMA (8.03 g, 92.2 mmol, 8.54 mL). The resulting mixture was stirred at 80° C. for 17 hours. The reaction mixture was cooled to room temperature, then added to the solution of NaIO₄ (11.79 g, 55.13 mmol) in H₂O (50 mL) at room temperature. The mixture was stirred at 25° C. for 17 hours. The solution was extracted with EtOAc (200 mL) and washed with water (50 mL*3). The organic phases were dried and concentrated in vacuo. The residue was purified by silica column (PE:EtOAc=30:1-1:1) to give 3-bromo-5-nitroisonicotinaldehyde (6.0 g, 26 mmol, 56% yield) as a yellow solid. ¹H NMR (400 MHz, DMSO-d₆) δ 10.24 (s, 1H), 9.37 (s, 1H), 9.29 (s, 1H).

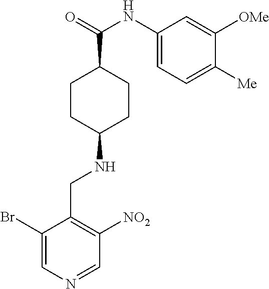 [SMILES: COc1cc(NC(=O)[C@H]2CC[C@@H](NCc3c(Br)cncc3[N+](=O)[O-])CC2)ccc1C]

### (1s,4s)-4-((3-bromo-5-nitropyridin-4-yl)methylamino)-N-(3-methoxy-4-methylphenyl)cyclohexanecarboxamide

To a solution of 3-bromo-5-nitro-pyridine-4-carbaldehyde (2.2 g, 9.5 mmol) in MeOH (10 mL) was added (1s,4s)-4-amino-N-(3-methoxy-4-methylphenyl)cyclohexanecarboxamide (2.25 g, 8.57 mmol). The solution was stirred at 25° C. for 17 hours. Sodium cyanoborohydride (1.8 g, 29 mmol) was added to the solution in portions. The resulting mixture was stirred at room temperature for another 16 hours. The mixture was poured into water (20 mL) and extracted with EtOAc (20 mL*3). The organic phases were dried and concentrated in vacuo. The residue was purified by silica column (PE:EtOAc=20:1 to 1:1) to give (1s,4s)-4-((3-bromo-5-nitropyridin-4-yl)methylamino)-N-(3-methoxy-4-methylphenyl)cyclohexanecarboxamide (2.0 g, 4.2 mmol, 44% yield) as a yellow solid. LCMS ESI m/z 479.1 [M+H]⁺.

### Step 3

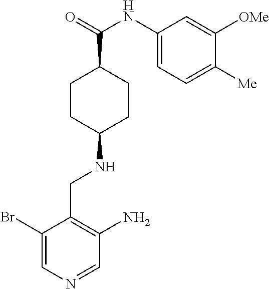 [SMILES: COc1cc(NC(=O)[C@H]2CC[C@@H](NCc3c(N)cncc3Br)CC2)ccc1C]

### (1s,4s)-4-((3-amino-5-bromopyridin-4-yl)methylamino)-N-(3-methoxy-4-methylphenyl)cyclohexanecarboxamide

To a solution of (1s,4s)-4-((3-bromo-5-nitropyridin-4-yl)methylamino)-N-(3-methoxy-4-methylphenyl)cyclohexanecarboxamide (350 mg, 730 μmol) in MeOH (10 mL) was added SnCl₂ (278 mg, 1.47 mmol). The mixture was stirred at 65° C. for 17 hours. The mixture was filtered through a celite pad, and the filtrate was poured into water (30 mL). The solution was extracted with EtOAc (30 mL*3), the organic phases were dried and concentrated in vacuo. The residue was purified by silica column (PE:EtOAc=20:1 to 1:1) to give (1s,4s)-4-((3-amino-5-bromopyridin-4-yl)methylamino)-N-(3-methoxy-4-methylphenyl)cyclohexanecarboxamide (200 mg, 450 μmol, 61% yield) as a yellow solid. LCMS ESI m/z 448.7 [M+H]⁺.

### Step 4

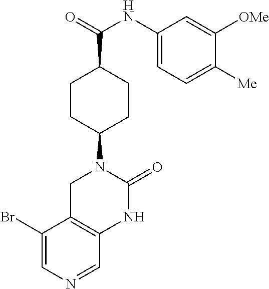 [SMILES: COc1cc(NC(=O)[C@H]2CC[C@@H](N3Cc4c(Br)cncc4NC3=O)CC2)ccc1C]

### (1s,4s)-4-(5-bromo-2-oxo-1,2-dihydropyrido[3,4-d]pyrimidin-3(4H)-yl)-N-(3-methoxy-4-methylphenyl)cyclohexanecarboxamide

To a solution (1s,4s)-4-((3-amino-5-bromopyridin-4-yl)methylamino)-N-(3-methoxy-4-methylphenyl)cyclohexanecarboxamide (180 mg, 402 μmol) in DCM (5 mL) was added triphosgene (107 mg, 362 μmol) and TEA (122 mg, 1.21 mmol, 168 μL). The resulting mixture was stirred at 25° C. for 17 hours. The mixture was quenched with water (10 mL) and extracted with EtOAc (10 mL*3). The organic layers were dried and concentrated. The residue was purified by prep-HPLC to give (1s,4s)-4-(5-bromo-2-oxo-1,2-dihydropyrido[3,4-d]pyrimidin-3(4H)-yl)-N-(3-methoxy-4-methylphenyl)cyclohexanecarboxamide (25 mg, 53 μmol, 13% yield) as a pink solid. LCMS ESI m/z 474.7 [M+H]⁺. ¹H NMR (400 MHz, DMSO-d₆) δ 9.70 (s, 1H), 9.62 (s, 1H), 8.21 (s, 1H), 7.98 (s, 1H), 7.28 (d, J=1.6 Hz, 1H), 7.11 (dd, J=8.0, 1.7 Hz, 1H), 7.02 (d, J=8.2 Hz, 1H), 4.34 (s, 2H), 4.26-4.13 (m, 1H), 3.75 (s, 3H), 2.66-2.64 (m, 1H), 2.22-1.86 (m, 7H), 1.68-1.64 (m, 2H), 1.50-1.46 (m, 2H).

### Example 92

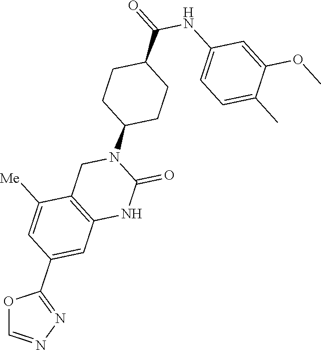 [SMILES: COc1cc(NC(=O)[C@H]2CC[C@@H](N3Cc4c(C)cc(-c5nnco5)cc4NC3=O)CC2)ccc1C]

### (1s,4s)-N-(3-methoxy-4-methylphenyl)-4-(5-methyl-7-(1,3,4-oxadiazol-2-yl)-2-oxo-1,2-dihydroquinazolin-3(4H)-yl)cyclohexanecarboxamide

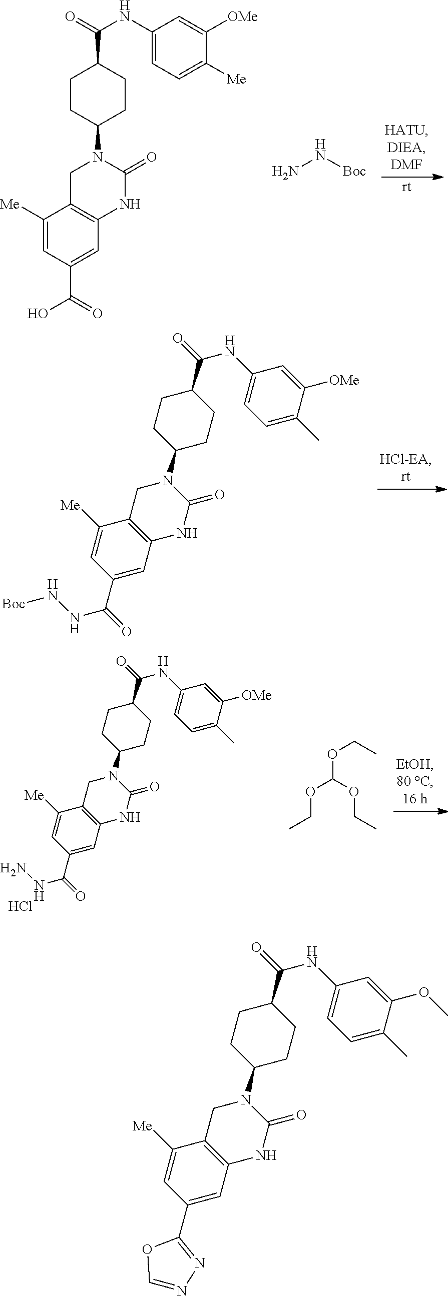

### Step 1

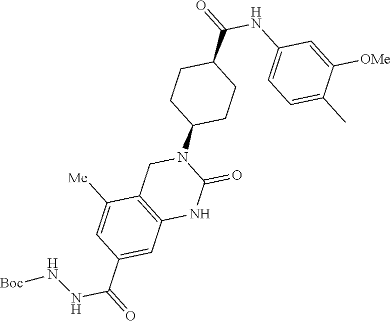 [SMILES: COc1cc(NC(=O)[C@H]2CC[C@@H](N3Cc4c(C)cc(C(=O)NNC(=O)OC(C)(C)C)cc4NC3=O)CC2)ccc1C]

### tert-butyl 2-(3-((1s,4s)-4-(3-methoxy-4-methylphenylcarbamoyl)cyclohexyl)-5-methyl-2-oxo-1,2,3,4-tetrahydroquinazoline-7-carbonyl)hydrazinecarboxylate

A solution of 3-[4-[(3-methoxy-4-methyl-phenyl)carbamoyl]cyclohexyl]-5-methyl-2-oxo-1,4-dihydroquinazoline-7-carboxylic acid (Example 79) (0.40 g, 890 μmol), DIPEA (343 mg, 2.66 mmol), HATU (371 mg, 975 μmol) and tert-butyl hydrazinecarboxylate (174 mg, 1.33 mmol) in DMF (15 mL) was stirred at room temperature overnight. The solution was poured into water and extracted by EtOAc, then washed with brine, dried by Na₂SO₄, filtered and concentrated to give tert-butyl 2-(3-((1s,4s)-4-(3-methoxy-4-methylphenylcarbamoyl)cyclohexyl)-5-methyl-2-oxo-1,2,3,4-tetrahydroquinazoline-7-carbonyl)hydrazinecarboxylate (260 mg, 52% yield) as a white solid. LCMS ESI m/z: 566.8 [M+H]⁺.

### Step 2

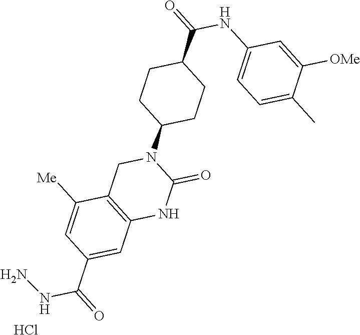 [SMILES: COc1cc(NC(=O)[C@H]2CC[C@@H](N3Cc4c(C)cc(C(=O)NN)cc4NC3=O)CC2)ccc1C ; Cl]

### (1s,4s)-4-(7-(hydrazinecarbonyl)-5-methyl-2-oxo-1,2-dihydroquinazolin-3(4H)-yl)-N-(3-methoxy-4-methylphenyl)cyclohexanecarboxamide hydrochloride

A solution of tert-butyl 2-(3-((1s,4s)-4-(3-methoxy-4-methylphenylcarbamoyl)cyclohexyl)-5-methyl-2-oxo-1,2,3,4-tetrahydroquinazoline-7-carbonyl)hydrazinecarboxylate (250 mg, 440 μmol) in EtOAc/HCl (3 mL) was stirred for 2 hours at 25° C. The mixture was concentrated to get (1s,4s)-4-(7-(hydrazinecarbonyl)-5-methyl-2-oxo-1,2-dihydroquinazolin-3(4H)-yl)-N-(3-methoxy-4-methylphenyl)cyclohexanecarboxamide hydrochloride (0.20 g, 430 μmol, 97% yield) as a yellow solid. The crude product was used directly in the next step. LCMS ESI m/z: 465.8 [M+H]⁺.

### Step 3

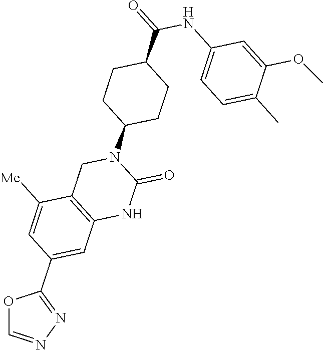 [SMILES: COc1cc(NC(=O)[C@H]2CC[C@@H](N3Cc4c(C)cc(-c5nnco5)cc4NC3=O)CC2)ccc1C]

### (1s,4s)-N-(3-methoxy-4-methylphenyl)-4-(5-methyl-7-(1,3,4-oxadiazol-2-yl)-2-oxo-1,2-dihydroquinazolin-3(4H)-yl)cyclohexanecarboxamide

A solution of (1s,4s)-4-(7-(hydrazinecarbonyl)-5-methyl-2-oxo-1,2-dihydroquinazolin-3(4H)-yl)-N-(3-methoxy-4-methylphenyl)cyclohexanecarboxamide hydrochloride (0.12 g, 260 μmol) and diethoxymethoxyethane (1.15 g, 7.73 mmol, 1.29 mL) in EtOH (6 mL) was stirred for 16 hours at 80° C. The mixture was concentrated and purified by flash chromatography on silica gel (0-10% EtOAc in PE; MeOH in DCM) to get the crude, which was purified by Prep-HPLC to get (1s,4s)-N-(3-methoxy-4-methylphenyl)-4-(5-methyl-7-(1,3,4-oxadiazol-2-yl)-2-oxo-1,2-dihydroquinazolin-3(4H)-yl)cyclohexanecarboxamide (0.0217 g, 45.6 μmol, 17.7% yield) as a white solid. LCMS ESI m/z: 475.8 [M+H]⁺. ¹H NMR (400 MHz, DMSO-d₆) δ 9.69 (s, 1H), 9.40 (s, 1H), 9.29 (s, 1H), 7.38 (s, 1H), 7.28 (d, J=10.0 Hz, 2H), 7.12 (d, J=8.0 Hz, 1H), 7.02 (d, J=8.0 Hz, 1H), 4.32 (s, 2H), 4.25-4.15 (m, 1H), 3.76 (s, 3H), 2.67-2.64 (m, 1H), 2.27 (s, 3H), 2.17-2.10 (m, 2H), 2.09 (s, 3H), 2.05-1.95 (m, 2H), 1.73-1.63 (m, 2H), 1.52-1.44 (m, 2H).

### Example 93

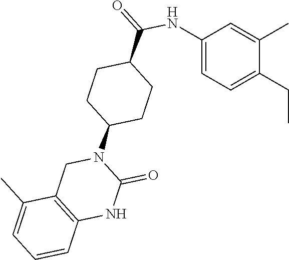 [SMILES: CCc1ccc(NC(=O)[C@H]2CC[C@@H](N3Cc4c(C)cccc4NC3=O)CC2)cc1C]

### (1s,4s)-N-(4-ethyl-3-methylphenyl)-4-(5-methyl-2-oxo-1,2-dihydroquinazolin-3(4H)-yl)cyclohexanecarboxamide

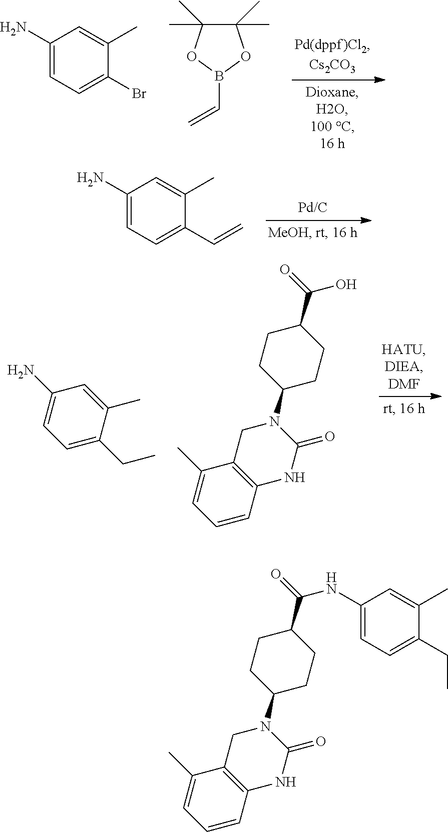

### Step 1

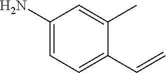

### 3-methyl-4-vinylaniline

To a solution of 4-bromo-3-methyl-aniline (500 mg, 2.69 mmol) and 4,4,5,5-tetramethyl-2-vinyl-1,3,2-dioxaborolane (497 mg, 3.22 mmol) in Water (1 mL) and 1,4-dioxane (5 mL) was added Cs₂CO₃ (1.75 g, 5.37 mmol) and Pd(dppf)Cl₂ (295 mg, 403 μmol). The reaction mixture was stirred at 100° C. for 16 hours under N₂. The mixture was purified by silica gel chromatography eluted with PE: EtOAc (0-30%) to give 3-methyl-4-vinyl-aniline (80 mg, 600 μmol, 22% yield) as yellow liquid. ¹H NMR (400 MHz, DMSO-d₆) δ 7.21 (d, J=8.3 Hz, 1H), 6.76 (dd, J=17.4, 11.0 Hz, 1H), 6.38 (dd, J=8.3, 2.3 Hz, 1H), 6.34 (d, J=2.2 Hz, 1H), 5.39 (dd, J=17.4, 1.7 Hz, 1H), 5.09 (s, 2H), 4.96 (dd, J=11.0, 1.6 Hz, 1H), 2.16 (s, 3H).

### Step 2

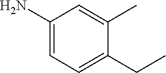

### 4-ethyl-3-methylaniline

To a solution 3-methyl-4-vinyl-aniline (80 mg, 600 μmol) in Methanol (5 mL) was added Pd/C (20 mg). The reaction mixture was stirred at 25° C. for 16 hours under H₂. The mixture was filter and then concentrated in vacuo to give 4-ethyl-3-methyl-aniline (60 mg, 440 μmol, 74% yield) as a yellow solid. ¹H NMR (400 MHz, DMSO-d₆) δ 6.76 (d, J=7.9 Hz, 1H), 6.35 (d, J=2.2 Hz, 1H), 6.32 (dd, J=7.9, 2.4 Hz, 1H), 4.68 (s, 2H), 2.40 (q, J=7.5 Hz, 2H), 2.10 (s, 3H), 1.06 (t, J=7.5 Hz, 3H).

### Step 3

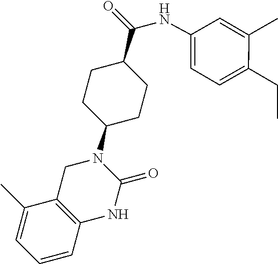

### (1s,4s)-N-(4-ethyl-3-methylphenyl)-4-(5-methyl-2-oxo-1,2-dihydroquinazolin-3(4H)-yl)cyclohexanecarboxamide

Following the synthetic procedure in Example 86, but starting with 4-ethyl-3-methylaniline gave the title compound as a white solid. LCMS (ESI) m/z: 406.3 [M+H]⁺. ¹H NMR (400 MHz, DMSO-d₆) δ 9.61 (s, 1H), 9.05 (s, 1H), 7.42-7.33 (m, 2H), 7.08-6.95 (m, 2H), 6.69 (d, J=7.5 Hz, 1H), 6.59 (d, J=7.9 Hz, 1H), 4.22-4.18 (m, 3H), 2.67-2.64 (m, 1H), 2.57-2.52 (m, 2H), 2.23 (s, 3H), 2.18-1.95 (m, 7H), 1.69-1.63 (m, 2H), 1.48-1.44 (m, 2H), 1.12 (t, J=7.5 Hz, 3H).

### Example 94

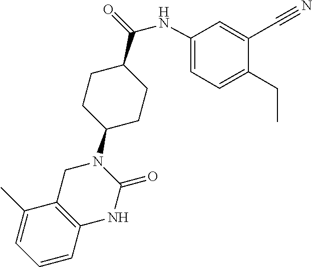 [SMILES: CCc1ccc(NC(=O)[C@H]2CC[C@@H](N3Cc4c(C)cccc4NC3=O)CC2)cc1C#N]

### (1s,4s)-N-(3-cyano-4-ethylphenyl)-4-(5-methyl-2-oxo-1,2-dihydroquinazolin-3(4H)-yl)cyclohexanecarboxamide

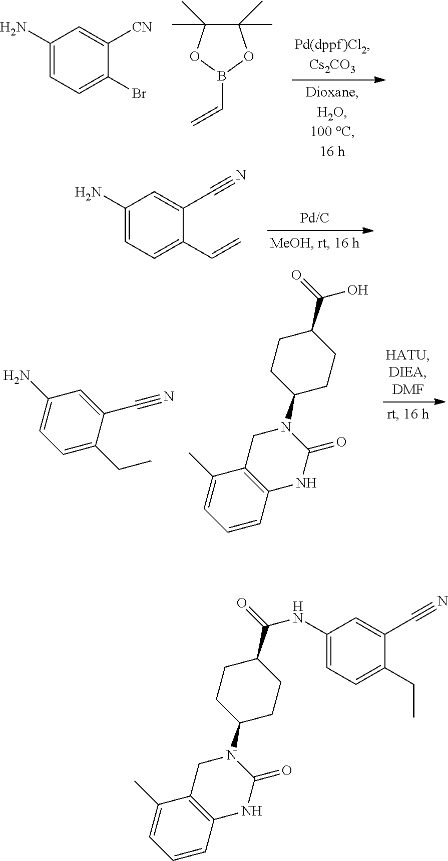

### Step 1, 2 and 3

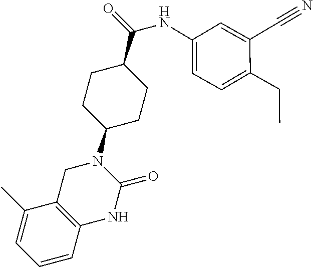 [SMILES: CCc1ccc(NC(=O)[C@H]2CC[C@@H](N3Cc4c(C)cccc4NC3=O)CC2)cc1C#N]

### (1s,4s)-N-(3-cyano-4-ethylphenyl)-4-(5-methyl-2-oxo-1,2-dihydroquinazolin-3(4H)-yl)cyclohexanecarboxamide

Following the synthetic procedure in Example 93, but starting with 5-amino-2-bromobenzonitrile gave the title compound as a yellow solid. LCMS (ESI) m/z: 417.2 [M+H]⁺. ¹H NMR (400 MHz, DMSO-d₆) 010.04 (s, 1H), 9.05 (s, 1H), 8.07 (d, J=2.2 Hz, 1H), 7.76 (dd, J=8.5, 2.3 Hz, 1H), 7.42 (d, J=8.5 Hz, 1H), 6.99 (t, J=7.7 Hz, 1H), 6.69 (d, J=7.5 Hz, 1H), 6.58 (d, J=7.9 Hz, 1H), 4.28-4.10 (m, 3H), 2.75 (q, J=7.6 Hz, 2H), 2.69-2.67 (m, 1H), 2.23-2.07 (m, 5H), 2.04-1.96 (m, 2H), 1.72-1.66 (m, 2H), 1.49-1.45 (m, 2H), 1.21 (t, J=7.6 Hz, 3H).

### Example 95

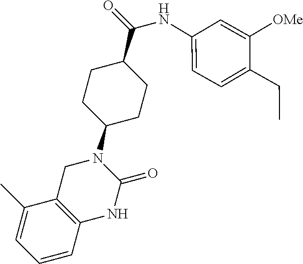 [SMILES: CCc1ccc(NC(=O)[C@H]2CC[C@@H](N3Cc4c(C)cccc4NC3=O)CC2)cc1OC]

### (1s,4s)-N-(4-ethyl-3-methoxyphenyl)-4-(5-methyl-2-oxo-1,2-dihydroquinazolin-3(4H)-yl)cyclohexanecarboxamide

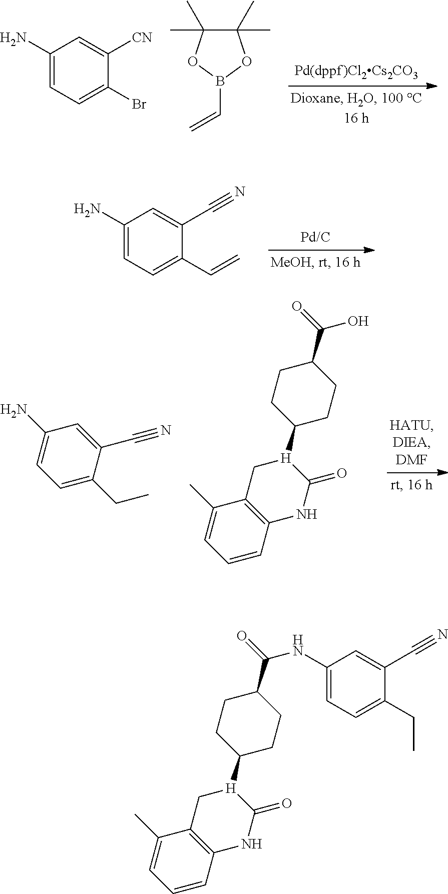

### Step 1, 2 and 3

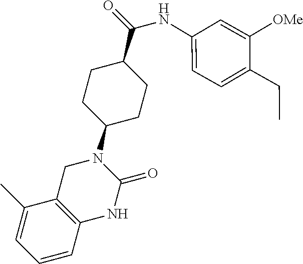 [SMILES: CCc1ccc(NC(=O)[C@H]2CC[C@@H](N3Cc4c(C)cccc4NC3=O)CC2)cc1OC]

### (1s,4s)-N-(4-ethyl-3-methoxyphenyl)-4-(5-methyl-2-oxo-1,2-dihydroquinazolin-3(4H)-yl)cyclohexanecarboxamide

Following the synthetic procedure in Example 93, but starting with 4-bromo-3-methoxyaniline gave the title compound as a yellow solid. LCMS (ESI) m/z: 421.9 [M+H]⁺. ¹H NMR (400 MHz, DMSO-d₆) δ 9.75 (s, 1H), 9.11 (s, 1H), 7.36 (d, J=1.5 Hz, 1H), 7.20 (dd, J=8.1, 1.6 Hz, 1H), 7.13-7.00 (m, 2H), 6.75 (d, J=7.5 Hz, 1H), 6.65 (d, J=7.9 Hz, 1H), 4.30-4.26 (m, 3H), 3.82 (s, 3H), 2.72-2.68 (m, 1H), 2.60-2.52 (m, 5H), 2.20-2.14 (m, 5H), 2.08-2.04 (m, 2H), 1.74-1.68 (m, 2H), 1.55-1.49 (m, 2H), 1.15 (t, J=7.5 Hz, 3H).

### Example 96

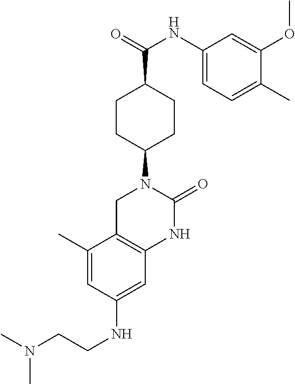

### (1s,4s)-4-(7-(2-(dimethylamino)ethylamino)-5-methyl-2-oxo-1,2-dihydroquinazolin-3(4H)-yl)-N-(3-methoxy-4-methylphenyl)cyclohexanecarboxamide

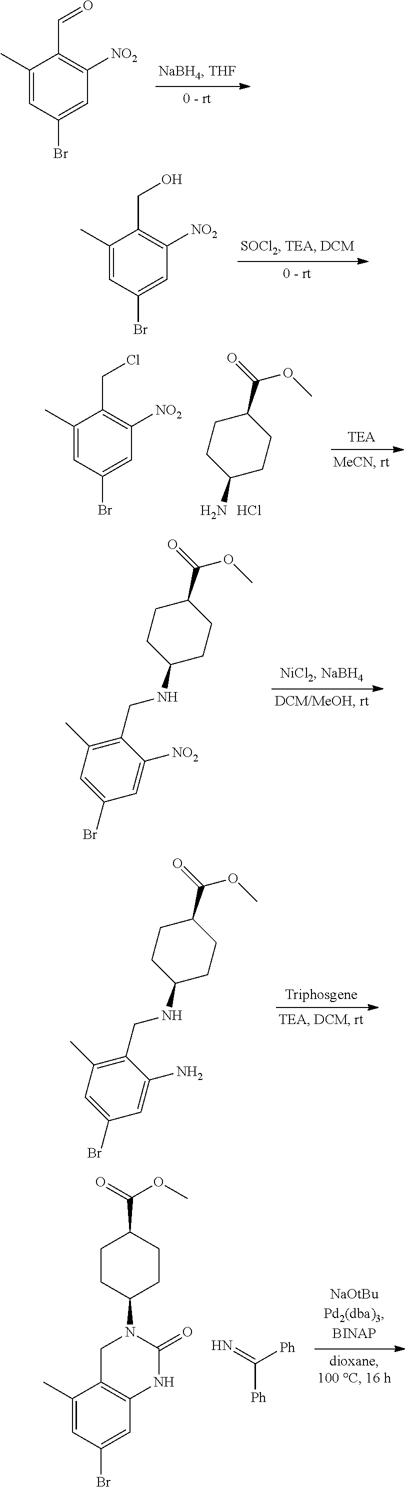

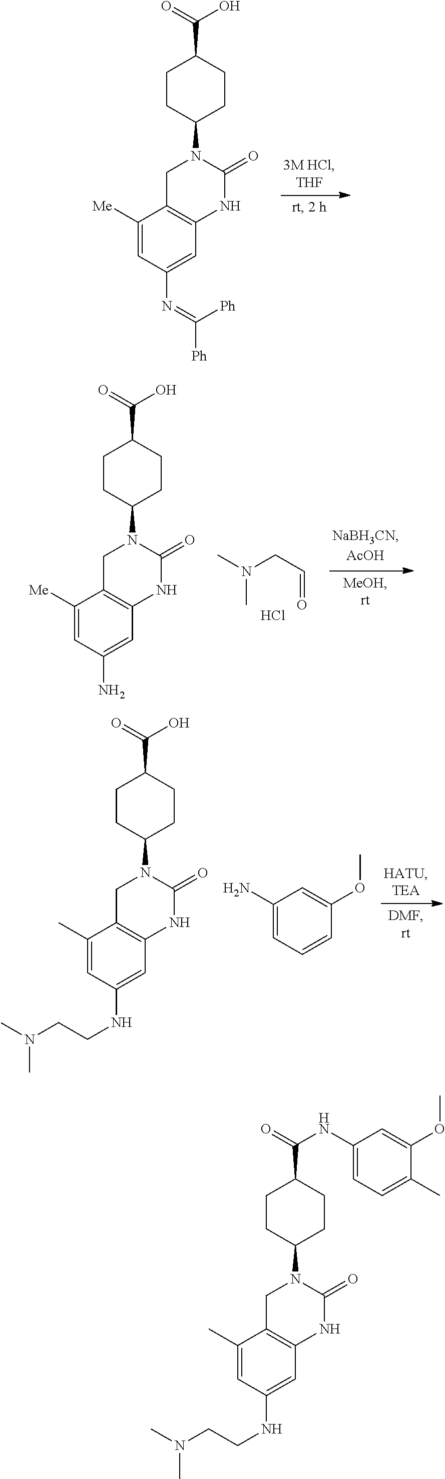

### Step 1

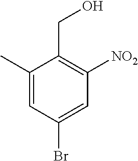

### (4-bromo-2-methyl-6-nitrophenyl)methanol

A solution of 4-bromo-2-methyl-6-nitro-benzaldehyde (13 g, 53 mmol) in THF (200 mL) was added NaBH₄ (1.42 g, 37.3 mmol). The mixture was stirred for 1 hour at 0° C. The reaction mixture was detected by TLC (PE:EtOAc=10:1, R_{f}=0.5). After starting material was consumed, it was quenched by saturation NH₄Cl (30 mL) at 25° C. and extracted with EtOAc (50 mL). The organic extract was dried over Na₂SO₄ and evaporated in vacuo. The residue was purified by flash chromatography on silica gel (0-10% EtOAc in PE) to get (4-bromo-2-methyl-6-nitro-phenyl) methanol (10 g, 41 mmol, 76% yield) as a white solid.

### Step 2

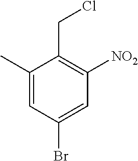

### 5-bromo-2-(chloromethyl)-1-methyl-3-nitrobenzene

To a solution of (4-bromo-2-methyl-6-nitro-phenyl) methanol (10 g, 41 mmol) and thionyl chloride (9.67 g, 81.3 mmol, 5.90 mL) in DCM (150 mL) was added dropwise N,N-diethylethanamine (2.06 g, 20.3 mmol, 2.83 mL) at 0° C. The mixture was warmed to room temperature and stirred for 1 h. The reaction mixture was poured into DCM (50 mL) and washed with NaHCO₃ (3×50 mL). The combined organic extracts were dried over Na₂SO₄ and evaporated in vacuo. The residue was purified by flash chromatography on silica gel (0-10% EtOAc in PE) to get 5-bromo-2-(chloromethyl)-1-methyl-3-nitro-benzene (10 g, 38 mmol, 93% yield) as a yellow oil. ¹H NMR (400 MHz, CDCl₃) δ 7.88 (d, J=1.6 Hz, 1H), 7.63 (d, J=1.6 Hz, 1H), 4.76 (s, 2H), 2.54 (s, 3H).

### Step 3

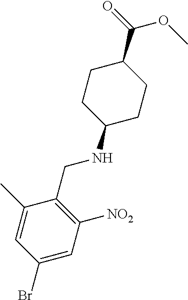

### (1s,4s)-methyl 4-(4-bromo-2-methyl-6-nitrobenzylamino)cyclohexanecarboxylate

A solution of 5-bromo-2-(chloromethyl)-1-methyl-3-nitro-benzene (5.0 g, 19 mmol), TEA (5.7 g, 57 mmol, 7.9 mL) and methyl 4-aminocyclohexanecarboxylate (5.49 g, 28.4 mmol, hydrochloride salt) in CH₃CN (100 mL) was stirred for 48 hours at 35° C. The reaction mixture was poured into EtOAc (50 mL) and washed with H₂O (3×50 mL). The combined organic extracts were dried over Na₂SO₄ and evaporated in vacuo. The residue was purified by flash chromatography on silica gel (0-10% EtOAc in PE) to get (1s,4s)-methyl 4-(4-bromo-2-methyl-6-nitrobenzylamino)cyclohexanecarboxylate (6.0 g, 16 mmol, 82% yield) as a white solid. LCMS ESI m/z: 386.7 [M+H]⁺.

### Step 4

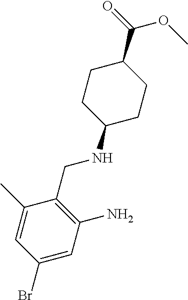

### (1s,4s)-methyl 4-(2-amino-4-bromo-6-methylbenzylamino)cyclohexanecarboxylate

A solution of (1s,4s)-methyl 4-(4-bromo-2-methyl-6-nitrobenzylamino)cyclohexanecarboxylate (5.5 g, 14 mmol) and dichloronickel hexahydrate (1.70 g, 7.14 mmol) in DCM (100 mL)/MeOH (20 mL) was added sodium borohydride (1.62 g, 42.8 mmol) at 0° C. The reaction mixture was stirred for 1 hour at 25° C. NaHCO₃ (50 mL) was added to the reaction mixture, and the solution was extracted with DCM (3×50 mL). The combined organic extracts were dried over Na₂SO₄ and evaporated in vacuo. The residue was purified by flash chromatography on silica gel (0-10% MeOH in DCM) to get (1s,4s)-methyl 4-(2-amino-4-bromo-6-methylbenzylamino)cyclohexanecarboxylate (4.5 g, 13 mmol, 89% yield) as a white solid. LCMS ESI m/z: 356.8 [M+H]⁺.

### Step 5

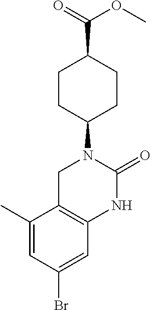

### (1s,4s)-methyl 4-(7-bromo-5-methyl-2-oxo-1,2-dihydroquinazolin-3(4H)-yl)cyclohexanecarboxylate

A solution of (1s,4s)-methyl 4-(2-amino-4-bromo-6-methylbenzylamino)cyclohexanecarboxylate (4.5 g, 13 mmol) and TEA (3.85 g, 38.0 mmol, 5.30 mL) in DCM (130 mL) at 0° C. was added bis(trichloromethyl)carbonate (2.63 g, 8.87 mmol). The mixture was stirred for 1 hour at 0° C. The mixture was poured into DCM (50 mL) and washed with NaHCO₃ (3×50 mL). The combined organic extracts were dried over Na₂SO₄ and evaporated in vacuo. The residue was purified by flash chromatography on silica gel (0-50% EtOAc in PE) to get (1s,4s)-methyl 4-(7-bromo-5-methyl-2-oxo-1,2-dihydroquinazolin-3(4H)-yl)cyclohexanecarboxylate (4.3 g, 11 mmol, 89% yield) as a white solid. LCMS ESI m/z: 382.7 [M+H]⁺.

### Step 6

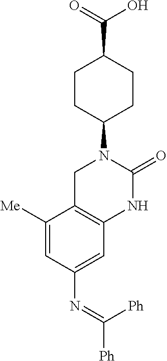 [SMILES: Cc1cc(N=C(c2ccccc2)c2ccccc2)cc2c1CN([C@H]1CC[C@@H](C(=O)O)CC1)C(=O)N2]

### (1s,4s)-4-(7-(diphenylmethyleneamino)-5-methyl-2-oxo-1,2-dihydroquinazolin-3(4H)-yl)cyclohexanecarboxylic Acid

A solution of diphenylmethanimine (4.56 g, 25.2 mmol, 4.23 mL), benzyl-[1-[2-[benzyl(phenyl)phosphanyl]-1-naphthyl]-2-naphthyl]-phenyl-phosphane (410 mg, 629 μmol), (1s,4s)-methyl 4-(7-bromo-5-methyl-2-oxo-1,2-dihydroquinazolin-3(4H)-yl)cyclohexanecarboxylate (2.4 g, 6.3 mmol), tBuONa (3.0 g, 31 mmol) and Pd₂(dba)₃ (410 mg, 0.63 mmol) in dioxane (20 mL) was stirred for 16 hours at 100° C. The reaction mixture was filtered and concentrated. The residue was diluted with EtOAc (50 mL) and washed with H₂O (3×50 mL). The combined organic extracts were dried over Na₂SO₄ and evaporated in vacuo. The residue was purified by flash chromatography on silica gel (0-5% MeOH in DCM) to get (1s,4s)-4-(7-(diphenylmethyleneamino)-5-methyl-2-oxo-1,2-dihydroquinazolin-3(4H)-yl)cyclohexanecarboxylic acid (2.8 g) as a yellow solid. LCMS (ESI) m/z: 586.8 [M+H]⁺. LCMS ESI m/z: 467.8 [M+H]⁺.

### Step 7

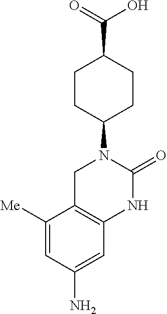 [SMILES: Cc1cc(N)cc2c1CN([C@H]1CC[C@@H](C(=O)O)CC1)C(=O)N2]

### (1s,4s)-4-(7-amino-5-methyl-2-oxo-1,2-dihydroquinazolin-3(4H)-yl)cyclohexanecarboxylic acid

To a solution of (1s,4s)-4-(7-(diphenylmethyleneamino)-5-methyl-2-oxo-1,2-dihydroquinazolin-3(4H)-yl)cyclohexanecarboxylic acid (2.8 g, 6.0 mmol) was added 3M HCl (15 mL). The mixture was stirred for 2 hours at 25° C. The solvent was evaporated in vacuo. The residue was diluted in EtOAc (50 mL) and washed with water (50 mL). The aqueous solution was freeze-dried to get the crude. The crude was stirred with DCM/MeOH (DCM:MeOH=10:1, 50 mL) and filtered through a celite pad. The filtrate was concentrated to give (1s,4s)-4-(7-amino-5-methyl-2-oxo-1,2-dihydroquinazolin-3(4H)-yl)cyclohexanecarboxylic acid (1.0 g, 3.3 mmol, 55% yield) as a yellow solid. LCMS ESI m/z: 303.9 [M+H]⁺.

### Step 8

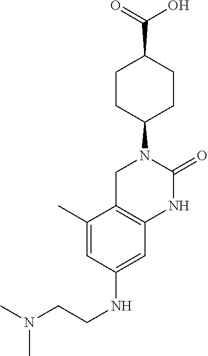 [SMILES: Cc1cc(NCCN(C)C)cc2c1CN([C@H]1CC[C@@H](C(=O)O)CC1)C(=O)N2]

### (1s,4s)-4-(7-(2-(dimethylamino)ethylamino)-5-methyl-2-oxo-1,2-dihydroquinazolin-3(4H)-yl)cyclohexanecarboxylic Acid

To a stirred solution of (1s,4s)-4-(7-amino-5-methyl-2-oxo-1,2-dihydroquinazolin-3(4H)-yl)cyclohexanecarboxylic acid (2.0 g, 6.6 mmol), 2-(dimethylamino)acetaldehyde hydrochloride (1.44 g, 16.5 mmol) and AcOH (396 mg, 6.59 mmol) at 25° C. in MeOH (20 mL) was added sodium cyanoborohydride (1.04 g, 16.5 mmol). The resulting mixture was stirred for 16 hours. The mixture was filtered through a celite pad, and the filtrate was concentrated. The residue was purified by flash chromatography on silica gel (0-20% MeOH in DCM) to get (1s,4s)-4-(7-(2-(dimethylamino)ethylamino)-5-methyl-2-oxo-1,2-dihydroquinazolin-3(4H)-yl)cyclohexanecarboxylic acid (1.0 g, 2.7 mmol, 41% yield) as a yellow solid. LCMS ESI m/z: 374.9 [M+H]⁺.

### Step 9

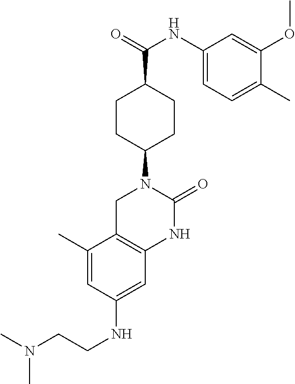

### (1s,4s)-4-(7-(2-(dimethylamino)ethylamino)-5-methyl-2-oxo-1,2-dihydroquinazolin-3(4H)-yl)-N-(3-methoxy-4-methylphenyl)cyclohexanecarboxamide

A solution of (1s,4s)-4-(7-(2-(dimethylamino)ethylamino)-5-methyl-2-oxo-1,2-dihydroquinazolin-3(4H)-yl)cyclohexanecarboxylic acid (0.15 g, 400 μmol), TEA (122 mg, 1.20 mmol, 167 μL), HATU (152 mg, 401 μmol) and 3-methoxy-4-methyl-aniline (55 mg, 400 μmol) in DMF (5 mL) was stirred for 2 hours at 25° C. The reaction mixture was poured into EtOAc (50 mL) and washed with H₂O (3×50 mL). The combined organic extracts were dried over Na₂SO₄ and evaporated in vacuo. The residue was purified by Prep-TLC (DCM:MeOH=5:1) to obtain crude product, which was purified by Prep-HPLC to get (1s,4s)-4-(7-(2-(dimethylamino)ethylamino)-5-methyl-2-oxo-1,2-dihydroquinazolin-3(4H)-yl)-N-(3-methoxy-4-methylphenyl)cyclohexanecarboxamide (0.0085 g, 14 μmol, 3.5% yield, triflouroacetate salt) as a white solid. LCMS ESI m/z: 493.9 [M+H]⁺. ¹H NMR (400 MHz, DMSO-d₆) δ 9.70 (s, 1H), 8.86 (s, 1H), 7.34-7.19 (m, 2H), 7.13-7.09 (m, 1H), 7.07-6.92 (m, 1H), 6.04 (d, J=1.6 Hz, 1H), 5.91 (d, J=2.0 Hz, 1H), 4.25-4.15 (m, 1H), 4.11 (s, 2H), 3.76 (s, 3H), 3.31-3.27 (m, 2H), 3.23-3.15 (m, 2H), 2.81 (s, 6H), 2.66-2.64 (m, 1H), 2.33-1.79 (m, 10H), 1.76-1.60 (m, 2H), 1.50-1.40 (m, 2H).

### Example 97

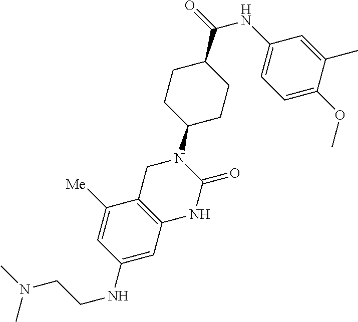

### (1s,4s)-4-(7-(2-(dimethylamino)ethylamino)-5-methyl-2-oxo-1,2-dihydroquinazolin-3(4H)-yl)-N-(4-methoxy-3-methylphenyl)cyclohexanecarboxamide

### Step 1

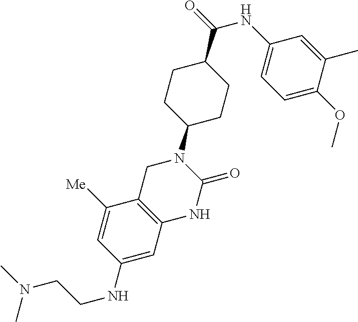

### (1s,4s)-4-(7-(2-(dimethylamino)ethylamino)-5-methyl-2-oxo-1,2-dihydroquinazolin-3(4H)-yl)-N-(4-methoxy-3-methylphenyl)cyclohexanecarboxamide

Following the synthetic procedure in Example 96, but starting with 4-methoxy-3-methylaniline gave the title compound as a white solid. LCMS (ESI) m/z: 493.9 [M+H]⁺. ¹H NMR (400 MHz, DMSO-d₆) δ 9.54 (s, 1H), 8.86 (s, 1H), 7.40-7.35 (m, 2H), 6.85 (d, J=9.6 Hz, 1H), 6.03 (s, 1H), 5.91 (s, 1H), 4.26-4.15 (m, 1H), 4.09 (s, 2H), 3.74 (s, 3H), 3.30-3.28 (m, 2H), 3.20-3.18 (t, J=6.0 Hz, 2H), 2.80 (s, 6H), 2.63-2.61 (m, 1H), 2.25-2.05 (m, 8H), 2.05-1.95 (m, 2H), 1.70-1.58 (m, 2H), 1.48-1.38 (m, 2H).

### Example 98

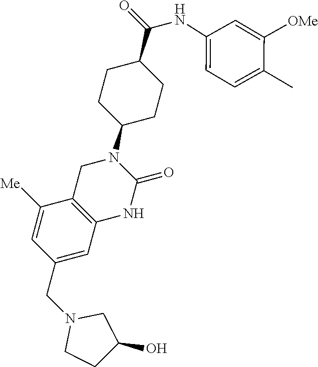 [SMILES: COc1cc(NC(=O)[C@H]2CC[C@@H](N3Cc4c(C)cc(CN5CC[C@H](O)C5)cc4NC3=O)CC2)ccc1C]

### (1R,4s)-4-(7-(((S)-3-hydroxypyrrolidin-1-yl)methyl)-5-methyl-2-oxo-1,2-dihydroquinazolin-3(4H)-yl)-N-(3-methoxy-4-methylphenyl)cyclohexanecarboxamide

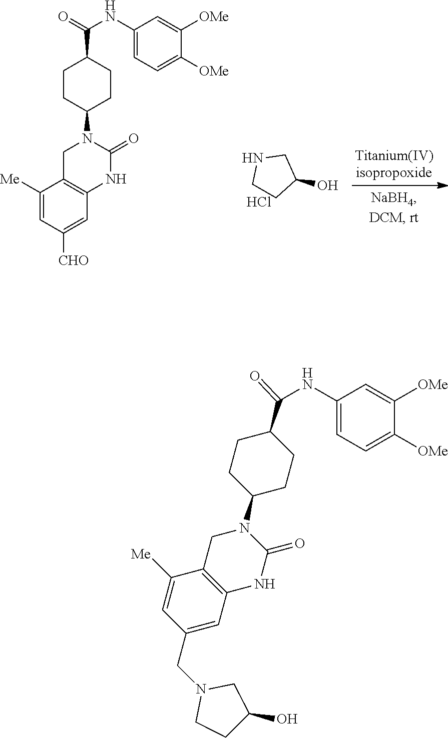 [SMILES: COc1ccc(NC(=O)[C@H]2CC[C@@H](N3Cc4c(C)cc(C=O)cc4NC3=O)CC2)cc1OC ; Cl ; O[C@H]1CCNC1 ; COc1ccc(NC(=O)[C@H]2CC[C@@H](N3Cc4c(C)cc(CN5CC[C@H](O)C5)cc4NC3=O)CC2)cc1OC]

### Step 1

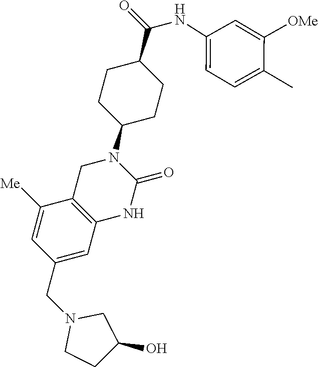 [SMILES: COc1cc(NC(=O)[C@H]2CC[C@@H](N3Cc4c(C)cc(CN5CC[C@H](O)C5)cc4NC3=O)CC2)ccc1C]

### (1R,4s)-4-(7-(((S)-3-hydroxypyrrolidin-1-yl)methyl)-5-methyl-2-oxo-1,2-dihydroquinazolin-3(4H)-yl)-N-(3-methoxy-4-methylphenyl)cyclohexanecarboxamide

To a solution of 4-(7-formyl-5-methyl-2-oxo-1,4-dihydroquinazolin-3-yl)-N-(3-methoxy-4-methyl-phenyl)cyclohexanecarboxamide (350 mg, 804 μmol) in DCM (2 mL) was added (3S)-pyrrolidin-3-ol (109 mg, 884 μmol, hydrochloride salt) and titanium (IV) isopropoxide (1.14 g, 4.02 mmol, 1.20 mL). The reaction mixture was stirred at 25° C. for 17 hours. To the reaction mixture was added NaBH₄ (36 mg, 0.96 mmol). After 30 mins, the mixture was concentrated under reduced pressure. The residue was purified by prep-HPLC to give (1R,4s)-4-(7-(((S)-3-hydroxypyrrolidin-1-yl)methyl)-5-methyl-2-oxo-1,2-dihydroquinazolin-3(4H)-yl)-N-(3-methoxy-4-methylphenyl)cyclohexanecarboxamide (14 mg, 26 μmol, 3.2% yield, formate salt) as a white solid. LCMS ESI m/z 507.3 [M+H]⁺. ¹H NMR (400 MHz, DMSO-d₆) δ 9.70 (s, 1H), 9.11 (s, 1H), 8.15 (s, 1H), 7.29 (d, J=1.6 Hz, 1H), 7.11 (dd, J=8.0, 1.7 Hz, 1H), 7.02 (d, J=8.2 Hz, 1H), 6.71 (s, 1H), 6.60 (s, 1H), 4.95 (b, 1H), 4.30-4.14 (m, 4H), 3.75 (s, 3H), 3.70-3.66 (m, 2H), 2.90-2.77 (m, 2H), 2.67-2.63 (m, 2H), 2.55-2.53 (m, 1H), 2.17-1.94 (m, 10H), 1.69-1.65 (m, 3H), 1.47-1.43 (m, 2H).

### Example 99

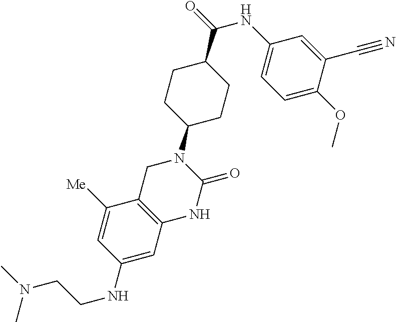 [SMILES: COc1ccc(NC(=O)[C@H]2CC[C@@H](N3Cc4c(C)cc(NCCN(C)C)cc4NC3=O)CC2)cc1C#N]

### (1s,4s)-N-(3-cyano-4-methoxyphenyl)-4-(7-(2-(dimethylamino)ethylamino)-5-methyl-2-oxo-1,2-dihydroquinazolin-3(4H)-yl)cyclohexanecarboxamide

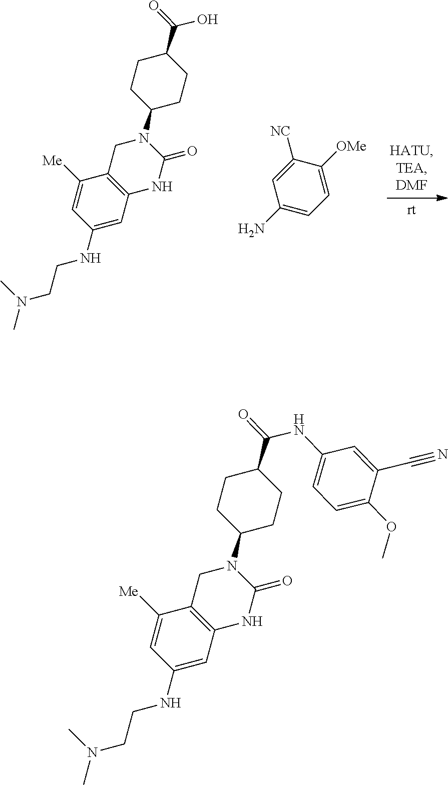 [SMILES: Cc1cc(NCCN(C)C)cc2c1CN([C@H]1CC[C@@H](C(=O)O)CC1)C(=O)N2 ; COc1ccc(N)cc1C#N ; COc1ccc(NC(=O)[C@H]2CC[C@@H](N3Cc4c(C)cc(NCCN(C)C)cc4NC3=O)CC2)cc1C#N]

### Step 1

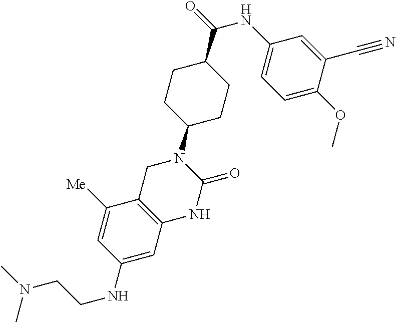 [SMILES: COc1ccc(NC(=O)[C@H]2CC[C@@H](N3Cc4c(C)cc(NCCN(C)C)cc4NC3=O)CC2)cc1C#N]

### (1s,4s)-N-(3-cyano-4-methoxyphenyl)-4-(7-(2-(dimethylamino)ethylamino)-5-methyl-2-oxo-1,2-dihydroquinazolin-3(4H)-yl)cyclohexanecarboxamide

Following the synthetic procedure in Example 96, but starting with 5-amino-2-methoxybenzonitrile gave the title compound as a white solid. LCMS (ESI) m/z: 504.9 [M+H]⁺. ¹H NMR (400 MHz, DMSO-d₆) 9.92 (s, 1H), 8.78 (s, 1H), 8.20 (b, 1H), 7.98 (d, J=2.4 Hz, 1H), 7.78 (dd, J=9.2, 2.4 Hz, 1H), 7.22 (d, J=9.2 Hz, 1H), 5.98 (s, 1H), 5.85 (s, 1H), 4.23-4.03 (m, 1H), 4.08 (s, 2H), 3.88 (s, 3H), 3.01 (t, J=6.4 Hz, 2H), 2.65-2.63 (m, 1H), 2.45 (t, J=6.4 Hz, 2H), 2.21 (s, 6H), 2.14-1.90 (m, 7H), 1.70-1.60 (m, 2H), 1.50-1.40 (m, 2H).

### Example 100

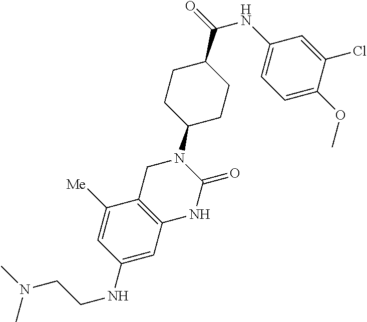 [SMILES: COc1ccc(NC(=O)[C@H]2CC[C@@H](N3Cc4c(C)cc(NCCN(C)C)cc4NC3=O)CC2)cc1Cl]

### (1s,4s)-N-(3-chloro-4-methoxyphenyl)-4-(7-(2-(dimethylamino)ethylamino)-5-methyl-2-oxo-1,2-dihydroquinazolin-3(4H)-yl)cyclohexanecarboxamide

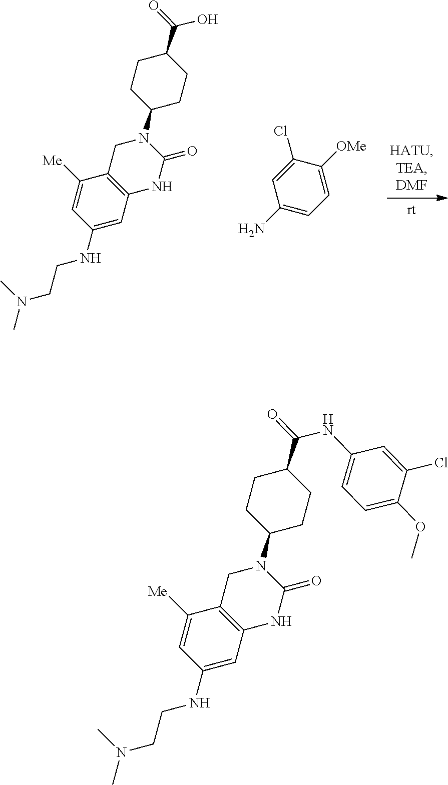 [SMILES: Cc1cc(NCCN(C)C)cc2c1CN([C@H]1CC[C@@H](C(=O)O)CC1)C(=O)N2 ; COc1ccc(N)cc1Cl ; COc1ccc(NC(=O)[C@H]2CC[C@@H](N3Cc4c(C)cc(NCCN(C)C)cc4NC3=O)CC2)cc1Cl]

### Step 1

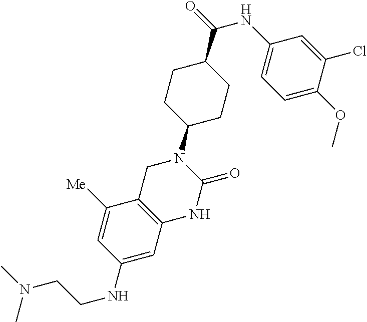 [SMILES: COc1ccc(NC(=O)[C@H]2CC[C@@H](N3Cc4c(C)cc(NCCN(C)C)cc4NC3=O)CC2)cc1Cl]

### (1s,4s)-N-(3-chloro-4-methoxyphenyl)-4-(7-(2-(dimethylamino)ethylamino)-5-methyl-2-oxo-1,2-dihydroquinazolin-3(4H)-yl)cyclohexanecarboxamide

Following the synthetic procedure in Example 96, but starting with 3-chloro-4-methoxyaniline gave the title compound as a white solid. LCMS (ESI) m/z: 513.8 [M+H]⁺. ¹H NMR (400 MHz, DMSO-d₆) δ 9.78 (s, 1H), 8.79 (s, 1H), 8.20 (s, 1H), 7.80 (d, J=2.4 Hz, 1H), 7.46 (dd, J=8.8, 2.4 Hz, 1H), 7.10 (d, J=9.2 Hz, 1H), 5.99 (s, 1H), 5.87 (d, J=1.6 Hz, 1H), 4.25-4.15 (m, 1H), 4.09 (s, 2H), 3.82 (s, 3H), 3.03 (t, J=6.4 Hz, 2H), 2.65-2.63 (m, 1H), 2.50-2.45 (m, 2H), 2.25 (s, 6H), 2.15-1.90 (m, 7H), 1.70-1.60 (m, 2H), 1.50-1.40 (m, 2H).

### Example 101 and Example 102

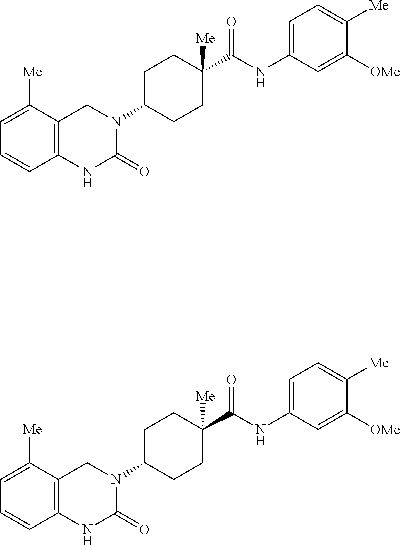 [SMILES: COc1cc(NC(=O)[C@]2(C)CC[C@H](N3Cc4c(C)cccc4NC3=O)CC2)ccc1C ; COc1cc(NC(=O)[C@]2(C)CC[C@@H](N3Cc4c(C)cccc4NC3=O)CC2)ccc1C]

### (1s,4s)-N-(3-methoxy-4-methylphenyl)-1-methyl-4-(5-methyl-2-oxo-1,2-dihydroquinazolin-3(4H)-yl)cyclohexanecarboxamide and (1r,4r)-N-(3-methoxy-4-methylphenyl)-1-methyl-4-(5-methyl-2-oxo-1,2-dihydroquinazolin-3(4H)-yl)cyclohexanecarboxamide

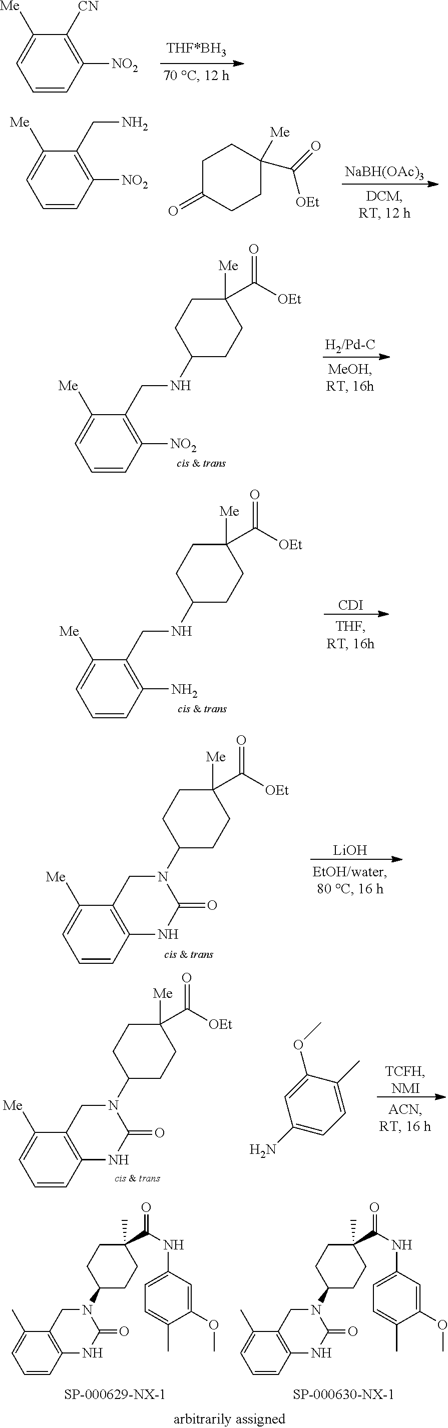

### Step 1

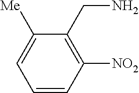 [SMILES: Cc1cccc([N+](=O)[O-])c1CN]

### (2-methyl-6-nitrophenyl)methanamine

A solution of 2-methyl-6-nitro-benzonitrile (3.0 g, 19 mmol) in BH₃-THF (5.43 mL, 55.5 mmol) was stirred at 70° C. for 12 hours. The reaction mixture was worked up with water and EtOAc. The organic layer was concentrated and purified by silica gel chromatograhy to give (2-methyl-6-nitro-phenyl)methanamine (1.2 g, 7.4 mmol, 40% yield) as a white solid. LCMS (ESI) m/z: 167.1 [M+H]⁺.

### Step 2

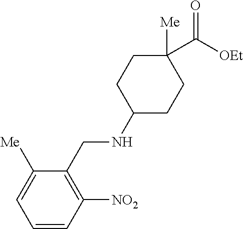 [SMILES: CCOC(=O)C1(C)CCC(NCc2c(C)cccc2[N+](=O)[O-])CC1]

### ethyl 1-methyl-4-(2-methyl-6-nitrobenzylamino)cyclohexanecarboxylate

A solution of (2-methyl-6-nitro-phenyl)methanamine (1.8 g, 11 mmol) in DCM (40 mL) was added ethyl 1-methyl-4-oxo-cyclohexanecarboxylate (2.00 g, 10.8 mmol). The reaction mixture was stirred at 25° C. for 1 hour. Sodium triacetoxyborohydride (4.59 g, 21.7 mmol) was added to the solution, and the mixture was stirred at 25° C. for 12 hours. The reaction mixture was poured into H₂O (10 mL) and extracted with EtOAc (10 mL*3). The combined organic layers were concentrated to the crude, then the crude product was purified by column (PE:EtOAc=2:1) to give ethyl 1-methyl-4-(2-methyl-6-nitrobenzylamino)cyclohexanecarboxylate (2.5 g, 7.5 mmol, 69% yield) as a white solid. LCMS (ESI) m/z: 355.2 [M+H]⁺.

### Step 3

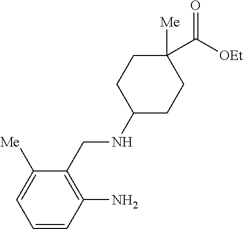 [SMILES: CCOC(=O)C1(C)CCC(NCc2c(C)cccc2N)CC1]

### ethyl 4-(2-amino-6-methylbenzylamino)-1-methylcyclohexanecarboxylate

To a solution of ethyl 1-methyl-4-(2-methyl-6-nitrobenzylamino)cyclohexanecarboxylate (2.8 g, 8.4 mmol) in MeOH (50 mL) was added Pd/C (150 mg). The reaction mixture was stirred under 15 psi hydrogen gas at 25° C. for 16 hours. The mixture was filtered and the filtrate concentrated to obtain ethyl 4-(2-amino-6-methylbenzylamino)-1-methylcyclohexanecarboxylate (2.5 g, 8.2 mmol, 98% yield) as a yellow solid, which was used directly without further purification. LCMS (ESI) m/z: 305.3 [M+H]⁺.

### Step 4

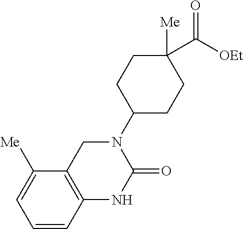 [SMILES: CCOC(=O)C1(C)CCC(N2Cc3c(C)cccc3NC2=O)CC1]

### ethyl 1-methyl-4-(5-methyl-2-oxo-1,2-dihydroquinazolin-3(4H)-yl)cyclohexanecarboxylate

The solution of ethyl 4-(2-amino-6-methylbenzylamino)-1-methylcyclohexanecarboxylate (2.5 g, 8.2 mmol) in THF (50 mL) was added 1,1′-carbonyldiimidazole (3.99 g, 24.6 mmol). The reaction mixture was stirred at 25° C. for 16 hours. The reaction mixture was concentrated, and the residue purified by chromatography on silica gel (PE/DCM=1/1) to give ethyl 1-methyl-4-(5-methyl-2-oxo-1,2-dihydroquinazolin-3(4H)-yl)cyclohexanecarboxylate (2.5 g, 7.6 mmol, 92% yield) as a white solid. LCMS (ESI) m/z: 331.2 [M+H]⁺.

### Step 5

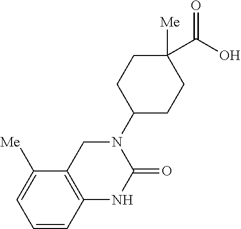 [SMILES: Cc1cccc2c1CN(C1CCC(C)(C(=O)O)CC1)C(=O)N2]

### 1-methyl-4-(5-methyl-2-oxo-1,2-dihydroquinazolin-3(4H)-yl)cyclohexanecarboxylic acid

To a solution of ethyl 1-methyl-4-(5-methyl-2-oxo-1,2-dihydroquinazolin-3(4H)-yl)cyclohexanecarboxylate (1.2 g, 3.6 mmol) in Ethanol (10 mL) was added LiOH (87 mg, 3.6 mmol) in water (10 mL). The reaction mixture was stirred at 80° C. for 16 hours. The mixture was acidified by 1M HCl to give 1-methyl-4-(5-methyl-2-oxo-1,2-dihydroquinazolin-3(4H)-yl)cyclohexanecarboxylic acid (1.0 g, 3.3 mmol, 91% yield) as a white solid. LCMS (ESI) m/z: 303.2 [M+H]⁺.

### Step 6

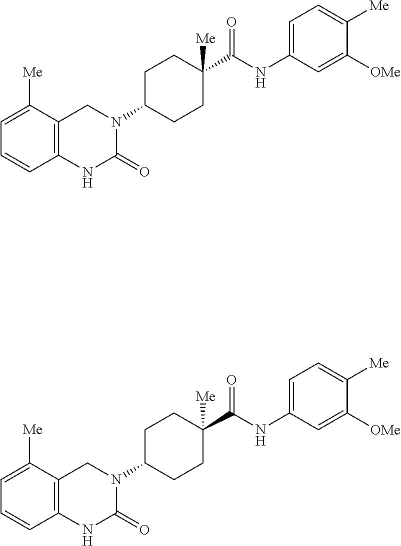 [SMILES: COc1cc(NC(=O)[C@]2(C)CC[C@H](N3Cc4c(C)cccc4NC3=O)CC2)ccc1C ; COc1cc(NC(=O)[C@]2(C)CC[C@@H](N3Cc4c(C)cccc4NC3=O)CC2)ccc1C]

### (1s,4s)-N-(3-methoxy-4-methylphenyl)-1-methyl-4-(5-methyl-2-oxo-1,2-dihydroquinazolin-3(4H)-yl)cyclohexanecarboxamide and (1r,4r)-N-(3-methoxy-4-methylphenyl)-1-methyl-4-(5-methyl-2-oxo-1,2-dihydroquinazolin-3(4H)-yl)cyclohexanecarboxamide

To a solution of 1-methyl-4-(5-methyl-2-oxo-1,2-dihydroquinazolin-3(4H)-yl)cyclohexanecarboxylic acid (100 mg, 330 μmol) in MeCN (2 mL) was added [chloro(dimethylamino)methylene]-dimethyl-ammonium hexafluorophosphate (111 mg, 397 μmol), 1-methylimidazole (95 mg, 1.2 mmol) and 3-methoxy-4-methylaniline (54 mg, 400 μmol), and then stirred at 25° C. for 16 hours. The mixture was filtered and concentrated. The residue was purified by Prep-HPLC to give the mixture of isomers (100 mg, 240 μmol, 72% yield) as a white solid. The isomers were separated by Chiral-SFC to give Peak 1 (1s,4s)-N-(3-methoxy-4-methylphenyl)-1-methyl-4-(5-methyl-2-oxo-1,2-dihydroquinazolin-3(4H)-yl)cyclohexanecarboxamide (20 mg) as a white solid (Example 101) and Peak 2 (1r,4r)-N-(3-methoxy-4-methylphenyl)-1-methyl-4-(5-methyl-2-oxo-1,2-dihydroquinazolin-3(4H)-yl)cyclohexanecarboxamide (25 mg) as a white solid (Example 102). The stereoisomeric assignment of Example 101 as (1s,4s) and Example 102 as (1r,4r) was arbitrary.

Example 101 LC/MS ESI 422.3 [M+H]⁺. ¹H NMR (400 MHz, DMSO-d₆) δ 9.25 (s, 1H), 9.04 (s, 1H), 7.32 (d, J=1.5 Hz, 1H), 7.19 (dd, J=8.1, 1.7 Hz, 1H), 7.06-6.94 (m, 2H), 6.67 (d, J=7.5 Hz, 1H), 6.57 (d, J=7.9 Hz, 1H), 4.22-4.19 (m, 1H), 4.15 (s, 2H), 3.77 (s, 3H), 2.42-1.38 (m, 2H), 2.10 (s, 3H), 2.09 (s, 3H), 1.74-1.70 (m, 2H), 1.52-1.48 (m, 2H), 1.34-1.30 (m, 2H), 1.21 (s, 3H).

Example 102 LC/MS ESI 422.3 [M+H]⁺. ¹H NMR (400 MHz, DMSO-d₆) δ 9.14 (s, 1H), 9.05 (s, 1H), 7.35 (d, J=1.7 Hz, 1H), 7.19 (dd, J=8.1, 1.8 Hz, 1H), 7.04-6.97 (m, 2H), 6.70 (d, J=7.5 Hz, 1H), 6.59 (d, J=7.8 Hz, 1H), 4.34 (s, 2H), 4.16-4.13 (m, 1H), 3.76 (s, 3H), 2.22 (s, 3H), 2.09 (s, 3H), 1.95-1.88 (m, 2H), 1.84-1.80 (m, 4H), 1.55-1.51 (m, 2H), 1.33 (s, 3H).

### Example 103

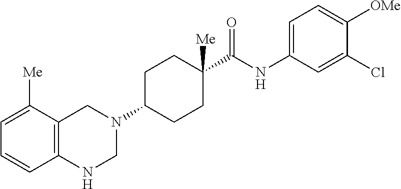 [SMILES: COc1ccc(NC(=O)[C@]2(C)CC[C@H](N3CNc4cccc(C)c4C3)CC2)cc1Cl]

### (1s,4s)-N-(3-chloro-4-methoxyphenyl)-1-methyl-4-(5-methyl-2-oxo-1,2-dihydroquinazolin-3(4H)-yl)cyclohexanecarboxamide

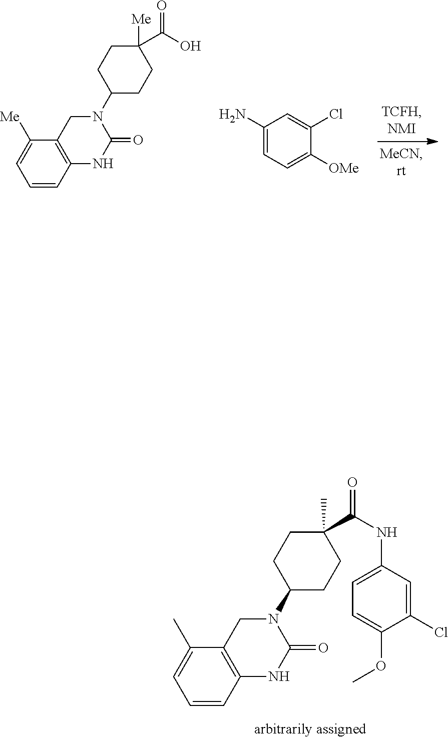 [SMILES: Cc1cccc2c1CN(C1CCC(C)(C(=O)O)CC1)C(=O)N2 ; COc1ccc(N)cc1Cl ; COc1ccc(NC(=O)[C@]2(C)CC[C@H](N3Cc4c(C)cccc4NC3=O)CC2)cc1Cl]

### Step 1

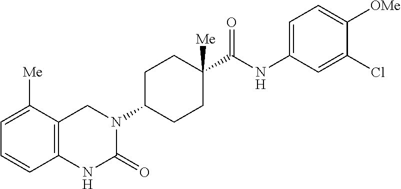 [SMILES: COc1ccc(NC(=O)[C@]2(C)CC[C@H](N3Cc4c(C)cccc4NC3=O)CC2)cc1Cl]

### (1s,4s)-N-(3-chloro-4-methoxyphenyl)-1-methyl-4-(5-methyl-2-oxo-1,2-dihydroquinazolin-3(4H)-yl)cyclohexanecarboxamide

Following the synthetic procedure for Example 101 and Example 102, but starting with 3-chloro-4-methoxyaniline gave the title compound as a white solid, isolated as the first peak from c-SFC. LCMS (ESI) m/z: 441.8 [M+H]⁺. ¹H NMR (400 MHz, DMSO-d₆) δ 9.35 (s, 1H), 9.04 (s, 1H), 7.79 (d, J=2.5 Hz, 1H), 7.54 (dd, J=8.9, 2.5 Hz, 1H), 7.12 (d, J=9.0 Hz, 1H), 6.98 (t, J=7.7 Hz, 1H), 6.67 (d, J=7.5 Hz, 1H), 6.58 (d, J=7.8 Hz, 1H), 4.23-4.12 (m, 3H), 3.83 (s, 3H), 2.40-2.36 (M, 2H), 2.10 (s, 3H), 1.73-1.68 (m, 2H), 1.53-1.48 (m, 2H), 1.35-1.31 (m, 2H), 1.20 (s, 3H). Example 103 was arbitrarily assigned as (1s,4s) stereoisomer.

### Example 104

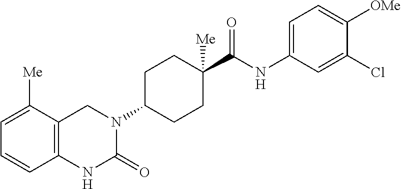 [SMILES: COc1ccc(NC(=O)[C@]2(C)CC[C@@H](N3Cc4c(C)cccc4NC3=O)CC2)cc1Cl]

### (1r,4r)-N-(3-chloro-4-methoxyphenyl)-1-methyl-4-(5-methyl-2-oxo-1,2-dihydroquinazolin-3(4H)-yl)cyclohexanecarboxamide

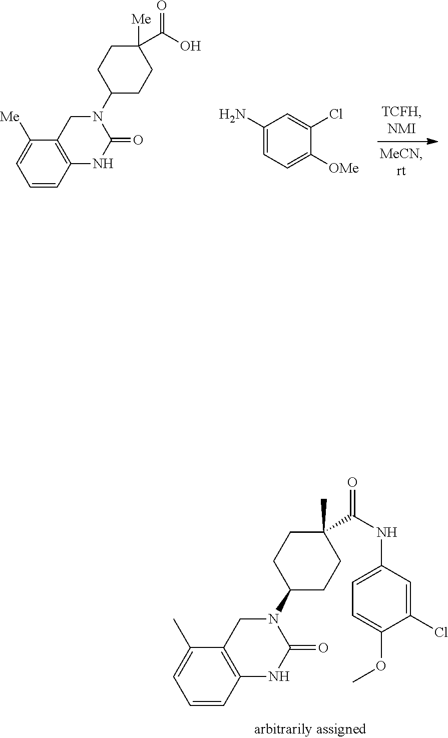 [SMILES: Cc1cccc2c1CN(C1CCC(C)(C(=O)O)CC1)C(=O)N2 ; COc1ccc(N)cc1Cl ; COc1ccc(NC(=O)[C@]2(C)CC[C@@H](N3Cc4c(C)cccc4NC3=O)CC2)cc1Cl]

### Step 1

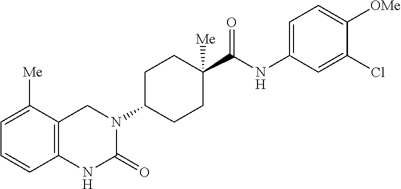 [SMILES: COc1ccc(NC(=O)[C@]2(C)CC[C@@H](N3Cc4c(C)cccc4NC3=O)CC2)cc1Cl]

### (1r,4r)-N-(3-chloro-4-methoxyphenyl)-1-methyl-4-(5-methyl-2-oxo-1,2-dihydroquinazolin-3(4H)-yl)cyclohexanecarboxamide

Following the synthetic procedure for Example 101 and Example 102, but starting with 3-chloro-4-methoxyaniline gave the title compound as a white solid, isolated as the second peak from c-SFC. LCMS (ESI) m/z: 441.8 [M+H]⁺. ¹H NMR (400 MHz, DMSO-d₆) δ 9.24 (s, 1H), 9.05 (s, 1H), 7.80 (d, J=2.5 Hz, 1H), 7.55 (dd, J=9.0, 2.5 Hz, 1H), 7.10 (d, J=9.1 Hz, 1H), 7.00 (t, J=7.7 Hz, 1H), 6.70 (d, J=7.5 Hz, 1H), 6.59 (d, J=7.8 Hz, 1H), 4.33 (s, 2H), 4.19-4.11 (m, 1H), 3.82 (s, 3H), 2.21 (s, 3H), 2.04-1.91 (m, 2H), 1.88-1.78 (m, 4H), 1.55-1.49 (m, 2H), 1.32 (s, 3H). Example 104 was arbitrarily assigned as (1r,4r) stereoisomer.

### Example 105

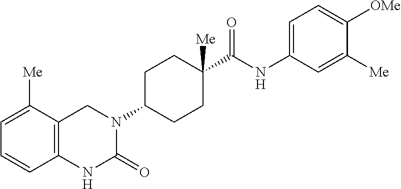 [SMILES: COc1ccc(NC(=O)[C@]2(C)CC[C@H](N3Cc4c(C)cccc4NC3=O)CC2)cc1C]

### (1s,4s)-N-(4-methoxy-3-methylphenyl)-1-methyl-4-(5-methyl-2-oxo-1,2-dihydroquinazolin-3(4H)-yl)cyclohexanecarboxamide

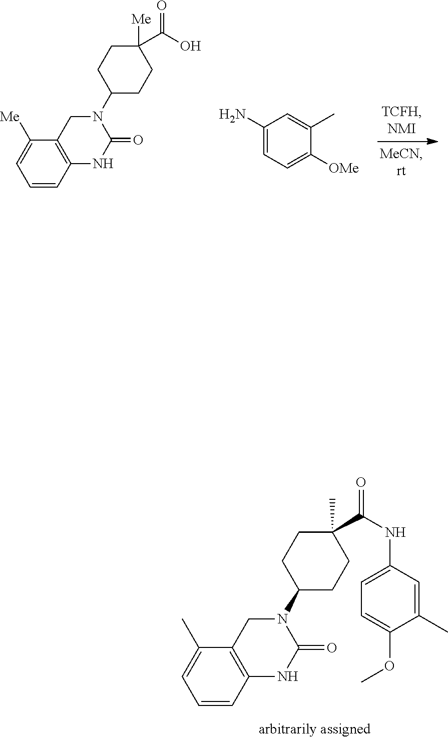

### Step 1

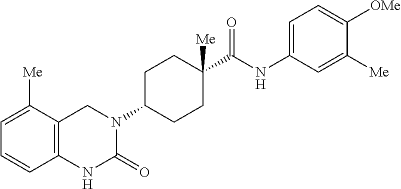 [SMILES: COc1ccc(NC(=O)[C@]2(C)CC[C@H](N3Cc4c(C)cccc4NC3=O)CC2)cc1C]

### (1s,4s)-N-(4-methoxy-3-methylphenyl)-1-methyl-4-(5-methyl-2-oxo-1,2-dihydroquinazolin-3(4H)-yl)cyclohexanecarboxamide

Following the synthetic procedure for Example 101 and Example 102, but starting with 4-methoxy-3-methylaniline gave the title compound as a white solid, isolated as the first peak from c-SFC. LCMS (ESI) m/z: 421.9 [M+H]⁺. ¹H NMR (400 MHz, DMSO-d₆) δ 9.15 (s, 1H), 9.04 (s, 1H), 7.43-7.32 (m, 2H), 6.98 (t, J=7.7 Hz, 1H), 6.87 (d, J=9.1 Hz, 1H), 6.67 (d, J=7.5 Hz, 1H), 6.58 (d, J=7.9 Hz, 1H), 4.25-4.10 (m, 3H), 3.76 (s, 3H), 2.40-2.36 (m, 2H), 2.14 (s, 3H), 2.10 (s, 3H), 1.75-1.68 (m, 2H), 1.53-1.47 (m, 2H), 1.35-1.27 (m, 2H), 1.19 (s, 3H). Example 105 was arbitrarily assigned as (1s,4s) stereoisomer.

### Example 106

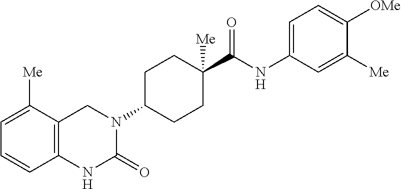 [SMILES: COc1ccc(NC(=O)[C@]2(C)CC[C@@H](N3Cc4c(C)cccc4NC3=O)CC2)cc1C]

### (1r,4r)-N-(4-methoxy-3-methylphenyl)-1-methyl-4-(5-methyl-2-oxo-1,2-dihydroquinazolin-3(4H)-yl)cyclohexanecarboxamide

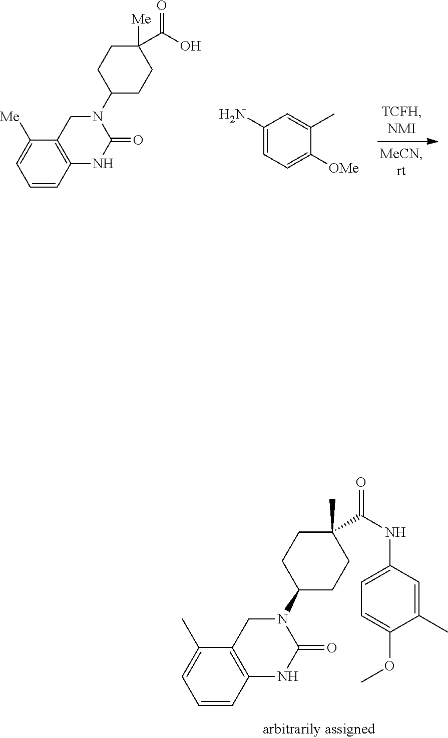

### Step 1

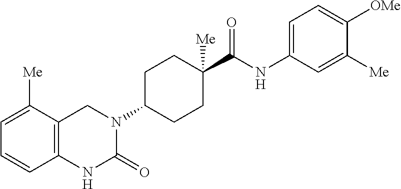 [SMILES: COc1ccc(NC(=O)[C@]2(C)CC[C@@H](N3Cc4c(C)cccc4NC3=O)CC2)cc1C]

### (1r,4r)-N-(4-methoxy-3-methylphenyl)-1-methyl-4-(5-methyl-2-oxo-1,2-dihydroquinazolin-3(4H)-yl)cyclohexanecarboxamide

Following the synthetic procedure for Example 101 and Example 102, but starting with 4-methoxy-3-methylaniline gave the title compound as a white solid, isolated as the second peak from c-SFC. LCMS (ESI) m/z: 421.9 [M+H]⁺. ¹H NMR (400 MHz, DMSO-d₆) δ 9.04 (d, J=7.4 Hz, 2H), 7.44-7.35 (m, 2H), 7.00 (t, J=7.7 Hz, 1H), 6.85 (d, J=8.6 Hz, 1H), 6.70 (d, J=7.5 Hz, 1H), 6.59 (d, J=7.8 Hz, 1H), 4.33 (s, 2H), 4.16-4.13 (m, 1H), 3.75 (s, 3H), 2.21 (s, 3H), 2.13 (s, 3H), 2.01-1.90 (m, 2H), 1.86-1.76 (m, 4H), 1.53-1.48 (m, 2H), 1.31 (s, 3H). Example 106 was arbitrarily assigned as (1r,4r) stereoisomer.

### Example 107

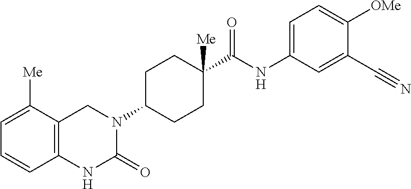 [SMILES: COc1ccc(NC(=O)[C@]2(C)CC[C@H](N3Cc4c(C)cccc4NC3=O)CC2)cc1C#N]

### (1s,4s)-N-(3-cyano-4-methoxyphenyl)-1-methyl-4-(5-methyl-2-oxo-1,2-dihydroquinazolin-3(4H)-yl)cyclohexanecarboxamide

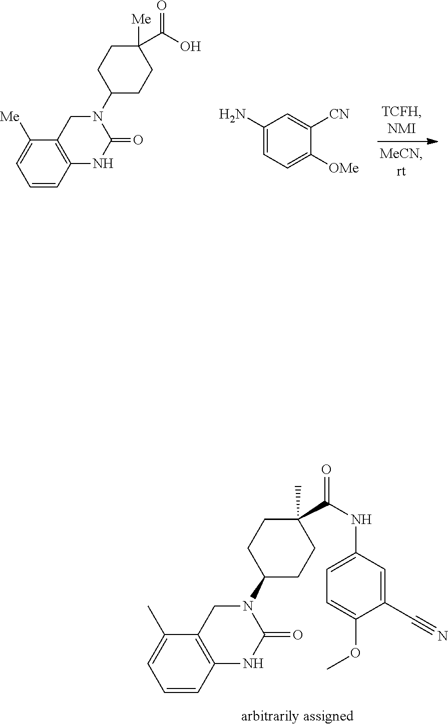 [SMILES: Cc1cccc2c1CN(C1CCC(C)(C(=O)O)CC1)C(=O)N2 ; COc1ccc(N)cc1C#N ; COc1ccc(NC(=O)[C@]2(C)CC[C@H](N3Cc4c(C)cccc4NC3=O)CC2)cc1C#N]

### Step 1

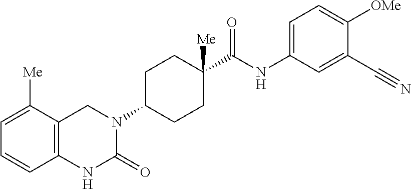 [SMILES: COc1ccc(NC(=O)[C@]2(C)CC[C@H](N3Cc4c(C)cccc4NC3=O)CC2)cc1C#N]

### (1s,4s)-N-(3-cyano-4-methoxyphenyl)-1-methyl-4-(5-methyl-2-oxo-1,2-dihydroquinazolin-3(4H)-yl)cyclohexanecarboxamide

Following the synthetic procedure for Example 101 and Example 102, but starting with 5-amino-2-methoxybenzonitrile gave the title compound as a white solid, isolated as the first peak from c-SFC. LCMS (ESI) m/z: 432.8 [M+H]⁺. ¹H NMR (400 MHz, DMSO-d₆) δ 9.49 (s, 1H), 9.04 (s, 1H), 7.99 (d, J=2.6 Hz, 1H), 7.86 (dd, J=9.2, 2.6 Hz, 1H), 7.25 (d, J=9.2 Hz, 1H), 6.98 (t, J=7.7 Hz, 1H), 6.67 (d, J=7.5 Hz, 1H), 6.57 (d, J=7.9 Hz, 1H), 4.22-4.13 (m, 3H), 3.90 (s, 3H), 2.41-2.36 (m, 2H), 2.10 (s, 3H), 1.78-1.66 (m, 2H), 1.54-1.49 (m, 2H), 1.37-1.31 (m, 2H), 1.20 (s, 3H). Example 107 was arbitrarily assigned as (1s,4s) stereoisomer.

### Example 108

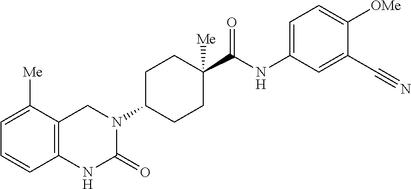 [SMILES: COc1ccc(NC(=O)[C@]2(C)CC[C@@H](N3Cc4c(C)cccc4NC3=O)CC2)cc1C#N]

### (1r,4r)-N-(3-cyano-4-methoxyphenyl)-1-methyl-4-(5-methyl-2-oxo-1,2-dihydroquinazolin-3(4H)-yl)cyclohexanecarboxamide

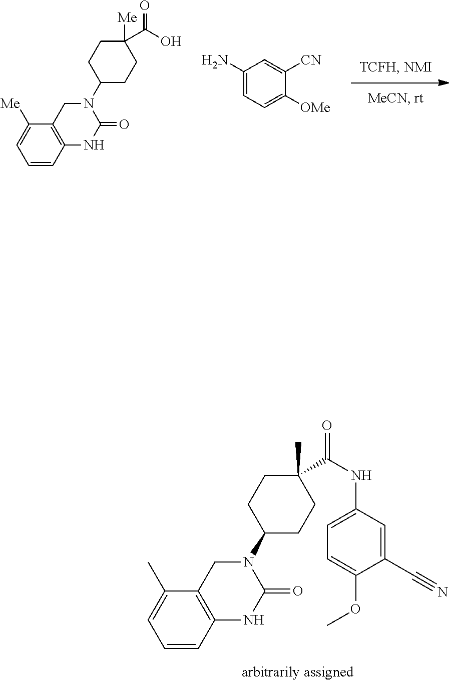 [SMILES: Cc1cccc2c1CN(C1CCC(C)(C(=O)O)CC1)C(=O)N2 ; COc1ccc(N)cc1C#N ; COc1ccc(NC(=O)[C@]2(C)CC[C@@H](N3Cc4c(C)cccc4NC3=O)CC2)cc1C#N]

### Step 1

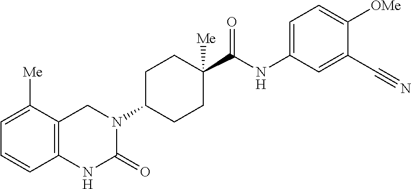 [SMILES: COc1ccc(NC(=O)[C@]2(C)CC[C@@H](N3Cc4c(C)cccc4NC3=O)CC2)cc1C#N]

### (1r,4r)-N-(3-cyano-4-methoxyphenyl)-1-methyl-4-(5-methyl-2-oxo-1,2-dihydroquinazolin-3(4H)-yl)cyclohexanecarboxamide

Following the synthetic procedure for Example 101 and Example 102, but starting with 5-amino-2-methoxybenzonitrile gave the title compound as a white solid, isolated as the second peak from c-SFC. LCMS (ESI) m/z: 432.9 [M+H]⁺. ¹H NMR (400 MHz, DMSO-d₆) δ 9.39 (s, 1H), 9.06 (s, 1H), 7.99 (s, 1H), 7.87 (d, J=9.2 Hz, 1H), 7.23 (d, J=9.2 Hz, 1H), 7.00 (t, J=7.6 Hz, 1H), 6.70 (d, J=7.3 Hz, 1H), 6.59 (d, J=7.8 Hz, 1H), 4.33 (s, 2H), 4.15-4.11 (m, 1H), 3.89 (s, 3H), 2.21 (s, 3H), 1.96-1.88 (m, 2H), 1.84-1.80 (m, 4H), 1.55-1.49 (m, 2H), 1.33 (s, 3H). Example 108 was arbitrarily assigned as (1r,4r) stereoisomer.

### Example 109

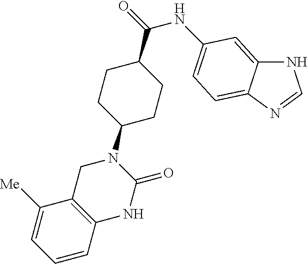 [SMILES: Cc1cccc2c1CN([C@H]1CC[C@@H](C(=O)Nc3ccc4nc[nH]c4c3)CC1)C(=O)N2]

### (1s,4s)-N-(1H-benzo[d]imidazol-6-yl)-4-(5-methyl-2-oxo-1,2-dihydroquinazolin-3(4H)-yl)cyclohexanecarboxamide

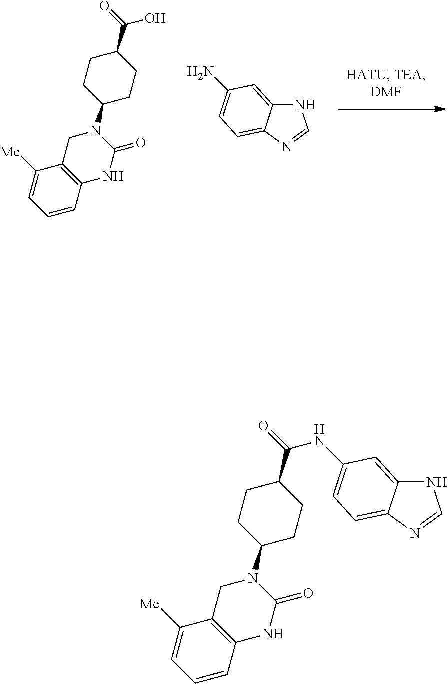 [SMILES: Cc1cccc2c1CN([C@H]1CC[C@@H](C(=O)O)CC1)C(=O)N2 ; Nc1ccc2nc[nH]c2c1 ; Cc1cccc2c1CN([C@H]1CC[C@@H](C(=O)Nc3ccc4nc[nH]c4c3)CC1)C(=O)N2]

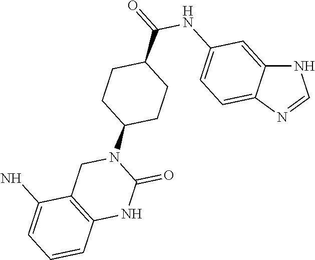 [SMILES: Nc1cccc2c1CN([C@H]1CC[C@@H](C(=O)Nc3ccc4nc[nH]c4c3)CC1)C(=O)N2]

### (1s,4s)-N-(1H-benzo[d]imidazol-6-yl)-4-(5-methyl-2-oxo-1,2-dihydroquinazolin-3(4H)-yl)cyclohexanecarboxamide

Following the general procedure above in Example 86, but starting with 1H-benzo[d]imidazol-6-amine gave the title compound as a white solid. LCMS (ESI) m/z: 404.2 [M+H]⁺. ¹H NMR (400 MHz, DMSO-d₆) δ 12.39 (b, 1H), 9.76 (s, 1H), 9.05 (s, 1H), 8.17-8.12 (m, 1H), 8.07 (d, J=1.6 Hz, 1H), 7.50 (d, J=8.8 Hz, 1H), 7.27 (dd, J=8.8, 1.6 Hz, 1H), 6.98 (t, J=7.6 Hz, 1H), 6.68 (d, J=7.6 Hz, 1H), 6.58 (d, J=8.0 Hz, 1H), 4.30-4.14 (m, 3H), 2.70-1.68 (m, 1H), 2.31-1.93 (m, 7H), 1.70-1.60 (m, 2H), 1.50-1.40 (m, 2H).

### Example 110

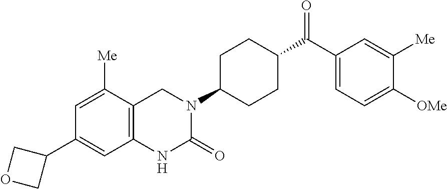 [SMILES: COc1ccc(C(=O)[C@H]2CC[C@H](N3Cc4c(C)cc(C5COC5)cc4NC3=O)CC2)cc1C]

### (1r,4r)-N-(3-methoxy-4-methylphenyl)-4-(5-methyl-7-(oxetan-3-yl)-2-oxo-1,2-dihydroquinazolin-3(4H)-yl)cyclohexanecarboxamide

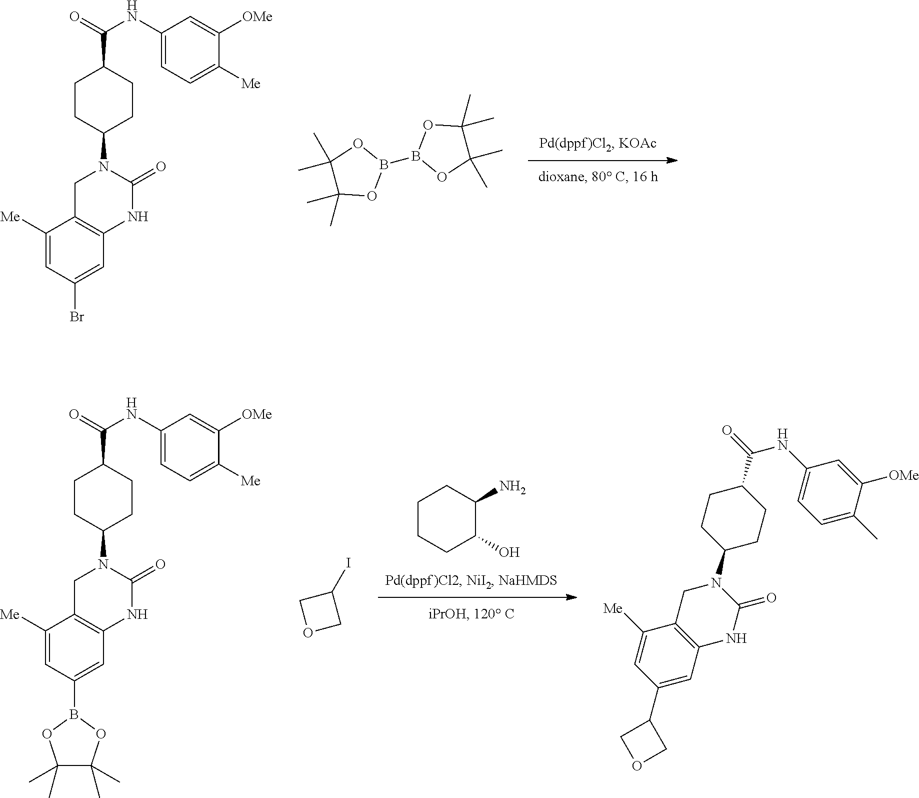

### Step 1

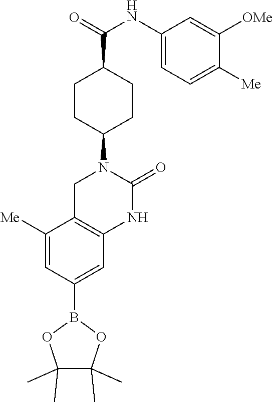 [SMILES: COc1cc(NC(=O)[C@H]2CC[C@@H](N3Cc4c(C)cc(B5OC(C)(C)C(C)(C)O5)cc4NC3=O)CC2)ccc1C]

### (1s,4s)-N-(3-methoxy-4-methylphenyl)-4-(5-methyl-2-oxo-7-(4,4,5,5-tetramethyl-1,3,2-dioxaborolan-2-yl)-1,2-dihydroquinazolin-3(4H)-yl)cyclohexanecarboxamide

A mixture of 4-(7-bromo-5-methyl-2-oxo-1,4-dihydroquinazolin-3-yl)-N-(3-methoxy-4-methyl-phenyl)cyclohexanecarboxamide (Example 74) (1.6 g, 3.3 mmol), 4,4,5,5-tetramethyl-2-(4,4,5,5-tetramethyl-1,3,2-dioxaborolan-2-yl)-1,3,2-dioxaborolane (1.00 g, 3.95 mmol), Pd(dppf)Cl₂ (241 mg, 329 μmol) and KOAc (646 mg, 6.58 mmol) in dioxane (70 mL) was stirred for 16 hours at 80° C. under N₂. The mixture was poured into EtOAc (50 mL) and washed with H₂O (3×50 mL). The combined organic extracts were dried over Na₂SO₄ and evaporated in vacuo. The residue was purified by flash chromatography on silica gel (0-10% MeOH in DCM) to get (1s,4s)-N-(3-methoxy-4-methylphenyl)-4-(5-methyl-2-oxo-7-(4,4,5,5-tetramethyl-1,3,2-dioxaborolan-2-yl)-1,2-dihydroquinazolin-3(4H)-yl)cyclohexanecarboxamide (1.3 g, 2.4 mmol, 74% yield) as a white solid. LCMS ESI m/z: 534.3 [M+H]⁺.

### Step 2

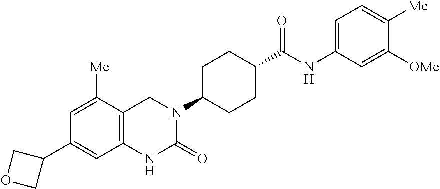 [SMILES: COc1cc(NC(=O)[C@H]2CC[C@H](N3Cc4c(C)cc(C5COC5)cc4NC3=O)CC2)ccc1C]

### (1r,4r)-N-(3-methoxy-4-methylphenyl)-4-(5-methyl-7-(oxetan-3-yl)-2-oxo-1,2-dihydroquinazolin-3(4H)-yl)cyclohexanecarboxamide

A mixture of (1s,4s)-N-(3-methoxy-4-methylphenyl)-4-(5-methyl-2-oxo-7-(4,4,5,5-tetramethyl-1,3,2-dioxaborolan-2-yl)-1,2-dihydroquinazolin-3(4H)-yl)cyclohexanecarboxamide (1.2 g, 2.3 mmol), 3-iodooxetane (828 mg, 4.50 mmol, 387 μL), nickel (II) iodide (70 mg, 220 μmol), (1R,2R)-2-aminocyclohexanol; hydrochloride (34 mg, 220 μmol) and sodium hexamethyldisilazane (830 mg, 4.50 mmol) in isopropyl alcohol (3 mL) was stirred for 16 hours at 120° C. under N₂. To the reaction mixture was added H₂O (50 mL) and extracted with EtOAc (3×50 mL). The combined organic extracts were dried over Na₂SO₄ and evaporated in vacuo. The residue was purified by Prep-TLC (DCM:MeOH=10:1) to get the crude product, which was purified by Prep-HPLC to give (1r,4r)-N-(3-methoxy-4-methylphenyl)-4-(5-methyl-7-(oxetan-3-yl)-2-oxo-1,2-dihydroquinazolin-3(4H)-yl)cyclohexanecarboxamide (0.024 g, 51 μmol, 2.3% yield) as a white solid. LCMS ESI m/z: 463.9 [M+H]⁺. ¹H NMR (400 MHz, DMSO-d₆) δ 9.74 (s, 1H), 9.08 (s, 1H), 7.36 (d, J=1.2 Hz, 1H), 7.10-6.96 (m, 2H), 6.75 (s, 1H), 6.62 (s, 1H), 4.95-4.85 (m, 2H), 4.57-4.44 (m, 2H), 4.28 (s, 2H), 4.18-4.01 (m, 2H), 3.75 (s, 3H), 2.35-2.25 (m, 1H), 2.20 (s, 3H), 2.08 (s, 3H), 2.00-1.90 (m, 2H), 1.80-1.65 (m, 4H), 1.63-1.50 (m, 2H).

### Example 111

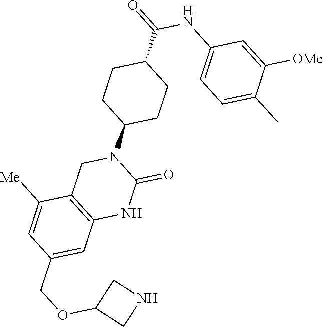 [SMILES: COc1cc(NC(=O)[C@H]2CC[C@H](N3Cc4c(C)cc(COC5CNC5)cc4NC3=O)CC2)ccc1C]

### (1r,4r)-4-(7-((azetidin-3-yloxy)methyl)-5-methyl-2-oxo-1,2-dihydroquinazolin-3(4H)-yl)-N-(3-methoxy-4-methylphenyl)cyclohexanecarboxamide

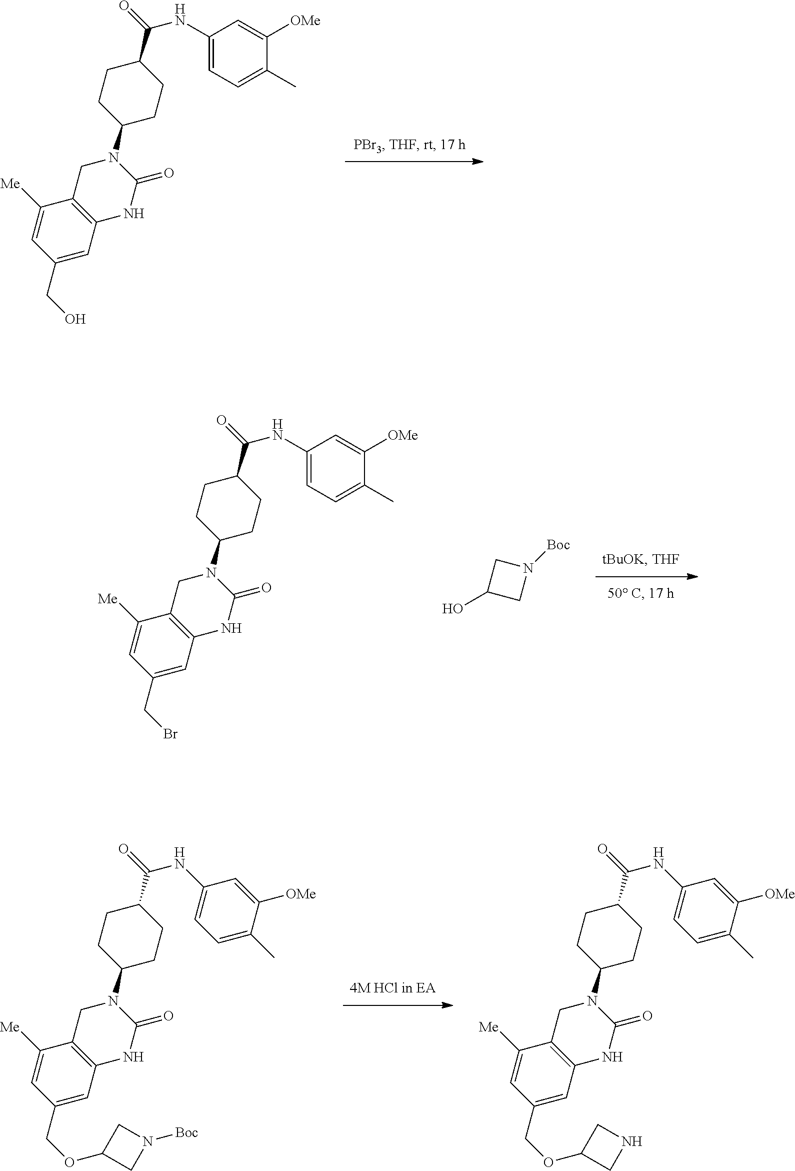 [SMILES: COc1cc(NC(=O)[C@H]2CC[C@@H](N3Cc4c(C)cc(CO)cc4NC3=O)CC2)ccc1C ; COc1cc(NC(=O)[C@H]2CC[C@@H](N3Cc4c(C)cc(CBr)cc4NC3=O)CC2)ccc1C ; CC(C)(C)OC(=O)N1CC(O)C1 ; COc1cc(NC(=O)[C@H]2CC[C@H](N3Cc4c(C)cc(COC5CN(C(=O)OC(C)(C)C)C5)cc4NC3=O)CC2)ccc1C ; COc1cc(NC(=O)[C@H]2CC[C@H](N3Cc4c(C)cc(COC5CNC5)cc4NC3=O)CC2)ccc1C]

### Step 1

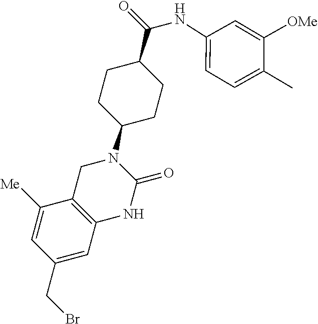 [SMILES: COc1cc(NC(=O)[C@H]2CC[C@@H](N3Cc4c(C)cc(CBr)cc4NC3=O)CC2)ccc1C]

### (1s,4s)-4-(7-(bromomethyl)-5-methyl-2-oxo-1,2-dihydroquinazolin-3(4H)-yl)-N-(3-methoxy-4-methylphenyl)cyclohexanecarboxamide

To a solution of 4-[7-(hydroxymethyl)-5-methyl-2-oxo-1,4-dihydroquinazolin-3-yl]-N-(3-methoxy-4-methyl-phenyl)cyclohexanecarboxamide (400 mg, 914 μmol) in THF (10 mL) was added PBr₃ (371 mg, 1.37 mmol) at room temperature. The resulting mixture was stirred at 25° C. for 17 hours. To the reaction mixture was added EtOAc (50 mL) and washed with H₂O (3×50 mL). The combined organic extracts were dried over Na₂SO₄ and evaporated in vacuo to give (1s,4s)-4-(7-(bromomethyl)-5-methyl-2-oxo-1,4-dihydroquinazolin-3(2H)-yl)-N-(3-methoxy-4-methylphenyl)cyclohexane-1-carboxamide (300 mg, 617 μmol, 67.5% yield) as a yellow solid. LCMS ESI m/z 500.1 [M+H]⁺.

### Step 2

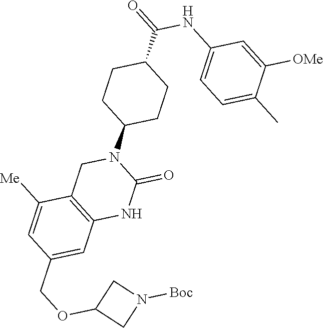 [SMILES: COc1cc(NC(=O)[C@H]2CC[C@H](N3Cc4c(C)cc(COC5CN(C(=O)OC(C)(C)C)C5)cc4NC3=O)CC2)ccc1C]

### tert-butyl 3-((3-((1r,4r)-4-(3-methoxy-4-methylphenylcarbamoyl)cyclohexyl)-5-methyl-2-oxo-1,2,3,4-tetrahydroquinazolin-7-yl)methoxy)azetidine-1-carboxylate

To a solution of (1s,4s)-4-(7-(bromomethyl)-5-methyl-2-oxo-1,2-dihydroquinazolin-3(4H)-yl)-N-(3-methoxy-4-methylphenyl)cyclohexanecarboxamide (500 mg, 999 μmol) in THF (10 mL) was added t-BuOK (336 mg, 3.00 mmol) and tert-butyl 3-hydroxyazetidine-1-carboxylate (346 mg, 2.00 mmol). The mixture was stirred at 50° C. for 17 hours. Epimerization of the amide alpha-carbon was observed during the course of the reaction, as determined by LCMS (vida infra). To the reaction mixture was added water (20 mL) and extracted with EtOAc (20 mL*3). The combined organic extracts were dried over Na₂SO₄ and evaporated in vacuo. The residue was purified by Prep-HPLC to get tert-butyl 3-((3-((1r,4r)-4-(3-methoxy-4-methylphenylcarbamoyl)cyclohexyl)-5-methyl-2-oxo-1,2,3,4-tetrahydroquinazolin-7-yl) methoxy)azetidine-1-carboxylate (100 mg, 0.228 mmol, 22.3% yield) as white solid. LCMS ESI m/z 492.9 [M−100+H]⁺.

### Step 3

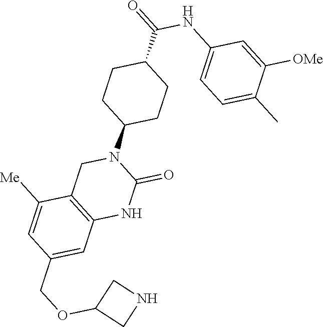 [SMILES: COc1cc(NC(=O)[C@H]2CC[C@H](N3Cc4c(C)cc(COC5CNC5)cc4NC3=O)CC2)ccc1C]

### (1r,4r)-4-(7-((azetidin-3-yloxy)methyl)-5-methyl-2-oxo-1,2-dihydroquinazolin-3(4H)-yl)-N-(3-methoxy-4-methylphenyl)cyclohexanecarboxamide

A solution of tert-butyl 3-[[3-[4-[(3-methoxy-4-methyl-phenyl)carbamoyl]cyclohexyl]-5-methyl-2-oxo-1,4-dihydroquinazolin-7-yl]methoxy]azetidine-1-carboxylate (60 mg, 100 μmol) in HCl/EtOAc (4 M, 10 mL) was stirred at 25° C. for 17 hours. The mixture was evaporated in vacuo, and the residue was purified by Prep-HPLC to get (1r,4r)-4-(7-((azetidin-3-yloxy)methyl)-5-methyl-2-oxo-1,2-dihydroquinazolin-3(4H)-yl)-N-(3-methoxy-4-methylphenyl)cyclohexanecarboxamide (7.0 mg, 14 μmol, 14% yield) as a white solid. LCMS ESI m/z 492.9 [M+H]⁺. ¹H NMR (400 MHz, DMSO-d₆) δ 9.76 (s, 1H), 9.10 (s, 1H), 7.36 (s, 1H), 7.07-6.93 (m, 2H), 6.69 (d, J=17.2 Hz, 1H), 6.58 (d, J=21.1 Hz, 1H), 4.37-4.24 (m, 5H), 4.15-4.10 (m, 1H), 3.81-3.72 (m, 5H), 3.62-3.56 (m, 3H), 2.33-2.28 (m, 1H), 2.19 (s, 3H), 2.07 (s, 3H), 1.96-1.92 (m, 2H), 1.79-1.70 (m, 4H), 1.59-1.50 (m, 2H).

### Example 112

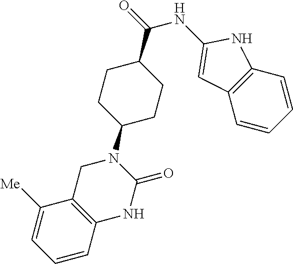 [SMILES: Cc1cccc2c1CN([C@H]1CC[C@@H](C(=O)Nc3cc4ccccc4[nH]3)CC1)C(=O)N2]

### (1s,4s)-N-(1H-indol-2-yl)-4-(5-methyl-2-oxo-1,2-dihydroquinazolin-3(4H)-yl)cyclohexanecarboxamide

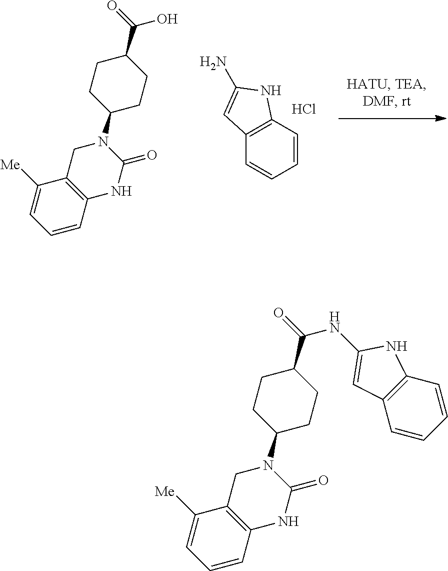 [SMILES: Cc1cccc2c1CN([C@H]1CC[C@@H](C(=O)O)CC1)C(=O)N2 ; Nc1cc2ccccc2[nH]1 ; Cl ; Cc1cccc2c1CN([C@H]1CC[C@@H](C(=O)Nc3cc4ccccc4[nH]3)CC1)C(=O)N2]

### Step 1

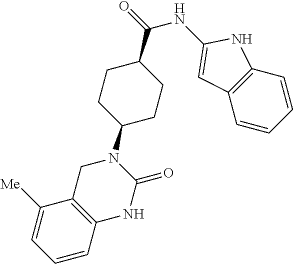 [SMILES: Cc1cccc2c1CN([C@H]1CC[C@@H](C(=O)Nc3cc4ccccc4[nH]3)CC1)C(=O)N2]

### (1s,4s)-N-(1H-indol-2-yl)-4-(5-methyl-2-oxo-1,2-dihydroquinazolin-3(4H)-yl)cyclohexanecarboxamide

Following the synthetic procedure for Example 86, but starting with 1H-indol-2-amine hydrochloride gave the title compound as a white solid. LCMS (ESI) m/z: 402.9 [M+H]⁺. ¹H NMR (400 MHz, DMSO-d₆) δ11.05 (s, 1H), 10.56 (s, 1H), 9.07 (s, 1H), 7.46-7.39 (m, 1H), 7.35 (dd, J=5.6, 2.8 Hz, 1H), 7.05-6.86 (m, 3H), 6.68 (d, J=7.6 Hz, 1H), 6.58 (d, J=8.0 Hz, 1H), 6.00 (d, J=1.6 Hz, 1H), 4.30-4.15 (m, 3H), 2.71-2.69 (m, 1H), 2.29-2.08 (m, 5H), 2.10-1.95 (m, 2H), 1.75-1.65 (m, 2H), 1.55-1.45 (m, 2H).

### Example 113

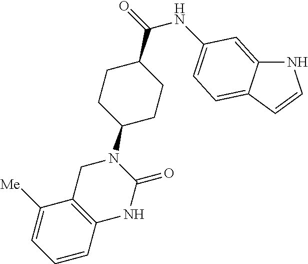 [SMILES: Cc1cccc2c1CN([C@H]1CC[C@@H](C(=O)Nc3ccc4cc[nH]c4c3)CC1)C(=O)N2]

### (1s,4s)-N-(1H-indol-6-yl)-4-(5-methyl-2-oxo-1,2-dihydroquinazolin-3(4H)-yl)cyclohexanecarboxamide

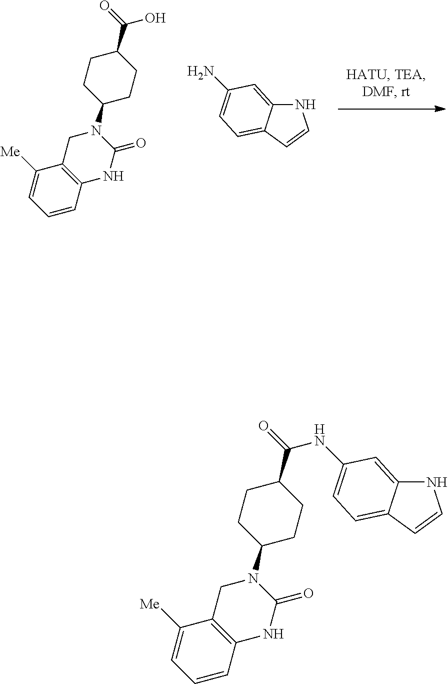 [SMILES: Cc1cccc2c1CN([C@H]1CC[C@@H](C(=O)O)CC1)C(=O)N2 ; Nc1ccc2cc[nH]c2c1 ; Cc1cccc2c1CN([C@H]1CC[C@@H](C(=O)Nc3ccc4cc[nH]c4c3)CC1)C(=O)N2]

### Step 1

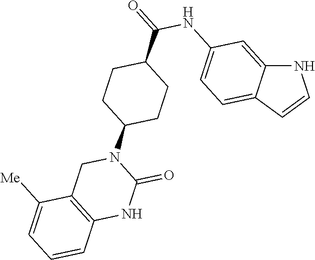 [SMILES: Cc1cccc2c1CN([C@H]1CC[C@@H](C(=O)Nc3ccc4cc[nH]c4c3)CC1)C(=O)N2]

### (1s,4s)-N-(1H-indol-6-yl)-4-(5-methyl-2-oxo-1,2-dihydroquinazolin-3(4H)-yl)cyclohexanecarboxamide

Following the synthetic procedure for Example 86, but starting with 1H-indol-6-amine gave the title compound as a white solid. LCMS (ESI) m/z: 403.2 [M+H]⁺. ¹H NMR (400 MHz, DMSO-d₆) δ 10.95 (s, 1H), 9.65 (s, 1H), 9.06 (s, 1H), 7.98 (s, 1H), 7.41 (d, J=8.5 Hz, 1H), 7.24 (t, J=2.6 Hz, 1H), 7.13-6.93 (m, 2H), 6.69 (d, J=7.5 Hz, 1H), 6.59 (d, J=7.9 Hz, 1H), 6.34 (s, 1H), 4.28-4.20 (m, 3H), 2.70-2.68 (m, 1H), 2.18-2.02 (m, 7H), 1.74-1.63 (m, 2H), 1.54-1.39 (m, 2H).

### Example 114

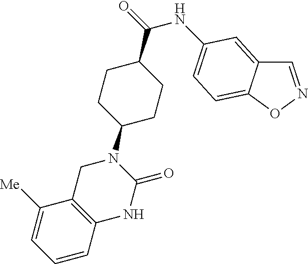 [SMILES: Cc1cccc2c1CN([C@H]1CC[C@@H](C(=O)Nc3ccc4oncc4c3)CC1)C(=O)N2]

### (1s,4s)-N-(benzo[d]isoxazol-5-yl)-4-(5-methyl-2-oxo-1,2-dihydroquinazolin-3(4H)-yl)cyclohexanecarboxamide

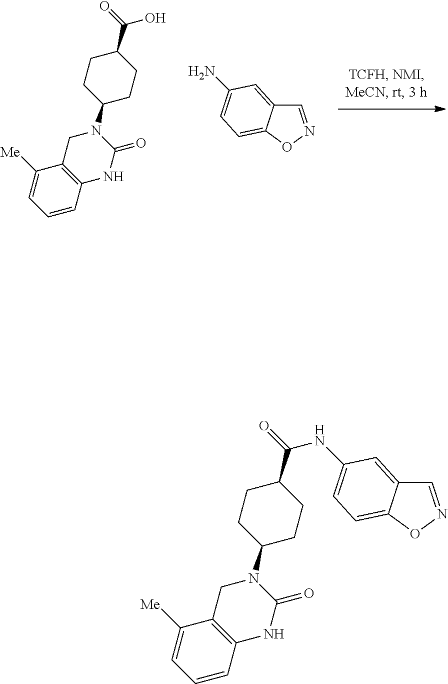 [SMILES: Cc1cccc2c1CN([C@H]1CC[C@@H](C(=O)O)CC1)C(=O)N2 ; Nc1ccc2oncc2c1 ; Cc1cccc2c1CN([C@H]1CC[C@@H](C(=O)Nc3ccc4oncc4c3)CC1)C(=O)N2]

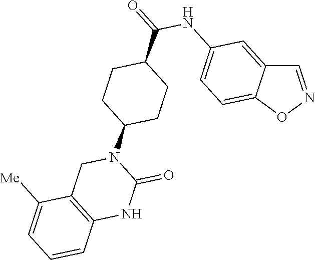 [SMILES: Cc1cccc2c1CN([C@H]1CC[C@@H](C(=O)Nc3ccc4oncc4c3)CC1)C(=O)N2]

### (1s,4s)-N-(benzo[d]isoxazol-5-yl)-4-(5-methyl-2-oxo-1,2-dihydroquinazolin-3(4H)-yl)cyclohexanecarboxamide

To a solution of 4-(5-methyl-2-oxo-1,4-dihydroquinazolin-3-yl)cyclohexanecarboxylic acid (70 mg, 240 μmol) in MeCN (5 mL) was added TCFH (11 mg, 290 μmol), 1-Methylimidazole (80 mg, 970 μmol, 77 μL) and 1,2-benzoxazol-5-amine (33 mg, 240 μmol). The mixture was stirred at 25° C. for 3 hours. To the mixture was added EtOAc (20 mL), washed with water (10 mL*3). The organic layers were dried and concentrated. The residue was purified by prep-HPLC to give (1s,4s)-N-(benzo[d]isoxazol-5-yl)-4-(5-methyl-2-oxo-1,2-dihydroquinazolin-3(4H)-yl)cyclohexanecarboxamide (19 mg, 44 μmol, 18% yield) as a white solid. LCMS ESI m/z: 404.9 [M+H]⁺. ¹H NMR (400 MHz, DMSO-d₆) δ 10.03 (s, 1H), 9.20 (s, 1H), 9.06 (s, 1H), 8.33 (s, 1H), 7.72 (s, 2H), 6.99 (t, J=7.7 Hz, 1H), 6.69 (d, J=7.5 Hz, 1H), 6.59 (d, J=7.9 Hz, 1H), 4.27-4.19 (m, 3H), 2.73-2.71 (m, 1H), 2.17-2.13 (m, 5H), 2.07-1.96 (m, 2H), 1.74-1.66 (m, 2H), 1.51-1.45 (m, 2H).

### Example 115

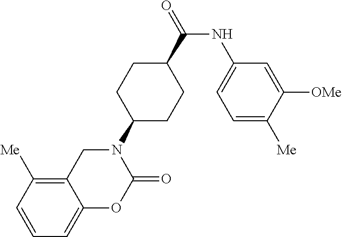 [SMILES: COc1cc(NC(=O)[C@H]2CC[C@@H](N3Cc4c(C)cccc4OC3=O)CC2)ccc1C]

### (1s,4s)-N-(3-methoxy-4-methylphenyl)-4-(5-methyl-2-oxo-2H-benzo[e][1,3]oxazin-3(4H)-yl)cyclohexane-1-carboxamide

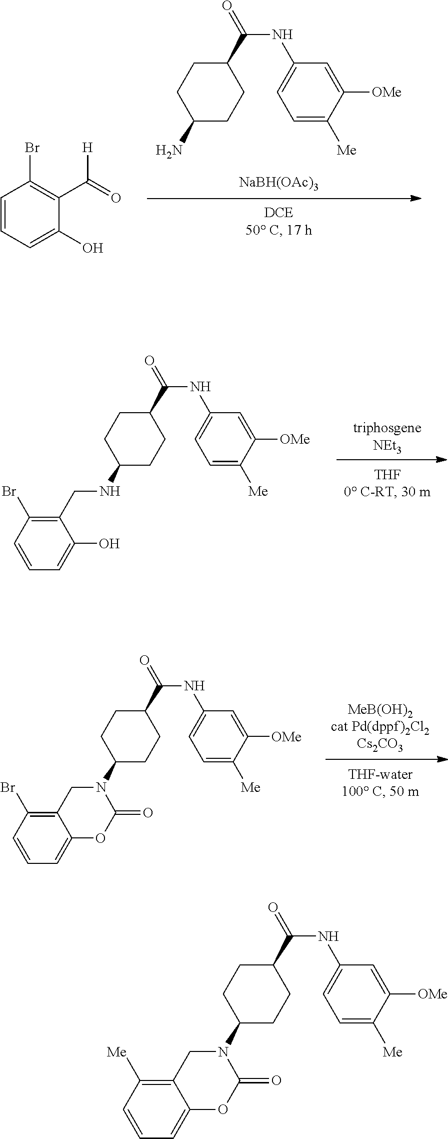

### Step 1

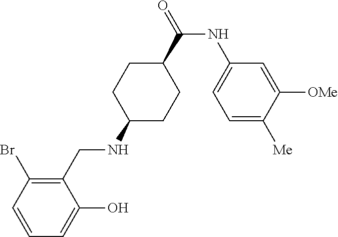 [SMILES: COc1cc(NC(=O)[C@H]2CC[C@@H](NCc3c(O)cccc3Br)CC2)ccc1C]

### (1s,4s)-4-((2-bromo-6-hydroxybenzyl)amino)-N-(3-methoxy-4-methylphenyl)cyclohexane-1-carboxamide

To a solution of 2-bromo-6-hydroxybenzaldehyde (275 mg, 1.34 mmol) and (1s,4s)-4-amino-N-(3-methoxy-4-methylphenyl)cyclohexanecarboxamide (352 mg, 1.34 mmol) in DCE (8.94 mL) was added a drop of acetic acid. The reagents were mixed for 1 hour at 50° C. Then triethylamine (282 mL, 2.01 mmol) was added to the reaction mixture and allowed to stir for additional 1.5 hours at 50° C. Sodium triacetoxyborohydride (849 mg, 4.02 mmol) was added in one portion and the reaction mixture was stirred at 50° C. overnight. A saturated solution of sodium bicarbonate was added to reaction mixture and it was extracted 3 times with ethyl acetate. Organics were combined, washed with brine, dried with anhydrous sodium sulfate, filtered and concentrated to dryness. The crude residue was purified by flash chromatography on silica gel (25 g column, 1 to 60% 20/80 MeOH/DCM in DCM) to yield (1s,4s)-4-((2-bromo-6-hydroxybenzyl)amino)-N-(3-methoxy-4-methylphenyl)cyclohexane-1-carboxamide (57.8 mg, 9.6% yield) as a white solid. LCMS ESI m/z: 447.1/449.1 (Br pattern) [M+H]⁺.

### Step 2

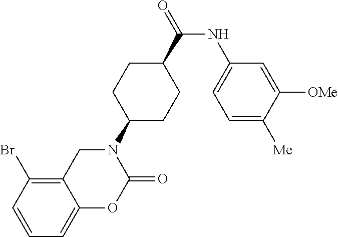 [SMILES: COc1cc(NC(=O)[C@H]2CC[C@@H](N3Cc4c(Br)cccc4OC3=O)CC2)ccc1C]

### (1s,4s)-4-(5-bromo-2-oxo-2H-benzo[e][1,3]oxazin-3(4H)-yl)-N-(3-methoxy-4-methylphenyl)cyclohexane-1-carboxamide

To a solution of (1s,4s)-4-((2-bromo-6-hydroxybenzyl)amino)-N-(3-methoxy-4-methylphenyl)cyclohexane-1-carboxamide (57.8 mg, 129 mmol) in THF (861 mL) at 0° C. was added triethylamine (27.1 mL, 194 mmol) followed by triphosgene (15.6 mg, 51.7 mmol). The resulting mixture was stirred at 0° C. for 5 min then at room temperature for 30 minutes. After 1 hour the reaction was complete by LCMS. The reaction was quenched with brine, extracted with EtOAc (×3) dried over anhydrous Na₂SO₄ and concentrated under reduced pressure to afford (1s,4s)-4-(5-bromo-2-oxo-2H-benzo[e][1,3]oxazin-3(4H)-yl)-N-(3-methoxy-4-methylphenyl)cyclohexane-1-carboxamide (61.2 mg, 129 mmol). LCMS ESI m/z: 474.1/476.0 (Br pattern) [M+H]⁺. The product was used without further purification in the subsequent step.

### Step 3

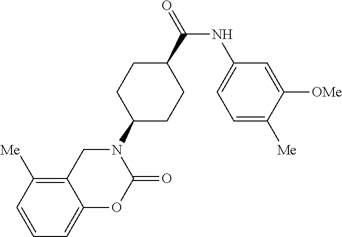 [SMILES: COc1cc(NC(=O)[C@H]2CC[C@@H](N3Cc4c(C)cccc4OC3=O)CC2)ccc1C]

### (1s,4s)-N-(3-methoxy-4-methylphenyl)-4-(5-methyl-2-oxo-2H-benzo[e][1,3]oxazin-3(4H)-yl)cyclohexane-1-carboxamide

A microwave vial was charged with 1,1′-bis(diphenylphosphino)ferrocene dichloropalladium (II) (4.83 mg, 6.46 mmol), cesium carbonate (129 mg, 388 mmol), methylboronic acid (7.74 mg, 129 mmol), (1s,4s)-4-(5-bromo-2-oxo-2H-benzo[e][1,3]oxazin-3(4H)-yl)-N-(3-methoxy-4-methylphenyl)cyclohexane-1-carboxamide (61.2 mg, 129 mmol), THF (1.18 mL) and water (118 mL). The vial was purged with nitrogen, capped and heated to 100° C. in a microwave reactor. After 50 mins, UPLC-MS confirmed the full conversion to desired product. The reaction mixture was cooled to room temperature and diluted with EtOAc (15 mL). This solution was washed with brine, dried over MgSO₄, filtered and the filtrate concentrated under reduced pressure. The residue was purified by flash column chromatography on silica gel using heptane and 0-80% EtOAc (with 1% i-PrOH additive in EtOAc) to afford (1s,4s)-N-(3-methoxy-4-methylphenyl)-4-(5-methyl-2-oxo-2H-benzo[e][1,3]oxazin-3(4H)-yl)cyclohexane-1-carboxamide (29.3 mg, 55% yield) as a white powder. LCMS ESI m/z: 409.2 [M+H]⁺. ¹H NMR (400 MHz, de-DMSO) δ 9.69 (s, 1H), 7.28 (d, J=1.1 Hz, 1H), 7.16 (t, J=8.0 Hz, 1H), 7.10 (dd, J=8.1, 1.3 Hz, 1H), 7.00 (d, J=8.4 Hz, 1H), 6.95 (d, J=7.5 Hz, 1H), 6.80 (d, J=8.1 Hz, 1H), 4.30 (s, 2H), 4.11-3.99 (m, 1H), 3.74 (s, 3H), 2.69-2.60 (m, 1H), 2.20 (s, 3H), 2.12 (d, J=13.8 Hz, 2H), 2.06 (s, 3H), 2.05-1.96 (m, 2H), 1.73-1.61 (m, 2H), 1.60-1.52 (m, 2H) ppm.

### Example 116

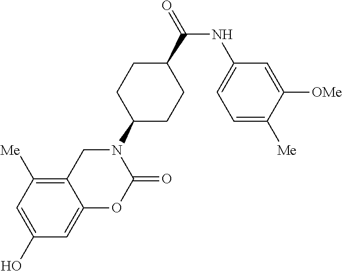 [SMILES: COc1cc(NC(=O)[C@H]2CC[C@@H](N3Cc4c(C)cc(O)cc4OC3=O)CC2)ccc1C]

### (1s,4s)-4-(7-hydroxy-5-methyl-2-oxo-2H-benzo[e][1,3]oxazin-3(4H)-yl)-N-(3-methoxy-4-methylphenyl)cyclohexane-1-carboxamide

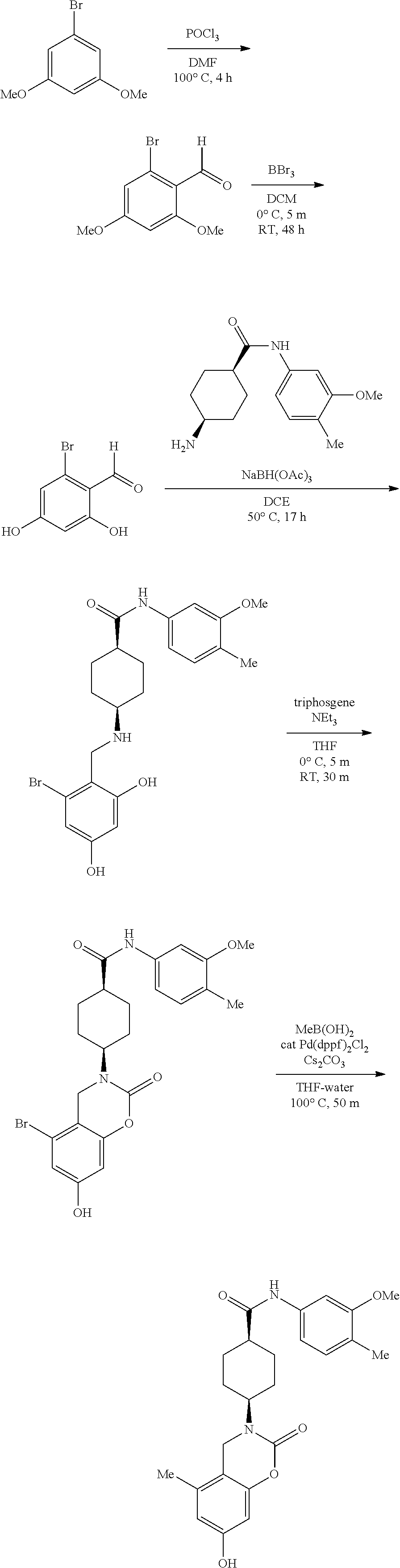

### Step 1

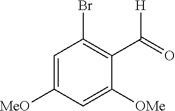 [SMILES: COc1cc(Br)c(C=O)c(OC)c1]

### 2-bromo-4,6-dimethoxybenzaldehyde

To a solution of 1-bromo-3,5-dimethoxybenzene (1.00 g, 4.61 mmol) dissolved in DMF (4.29 mL, 55.3 mmol) at 0° C. was added phosphorus oxychloride (1.30 mL, 13.8 mmol) dropwise, and the resulting mixture was allowed to stir at room temperature for 30 minutes. The mixture was heated to 100° C. for 4 hours then poured into ice cold water and further quenched by the slow addition of solid KOH until pH~14 was obtained. The resulting mixture was stirred for 16 hours at room temperature, filtered and the filter cake was dissolved in diethyl ether and concentrated under reduced pressure to afford 2-bromo-4,6-dimethoxybenzaldehyde (1.13 g, 4.61 mmol, 87% yield). ¹H NMR (400 MHz, CDCl₃) δ 10.31 (s, 1H), 6.78 (d, J=2.2 Hz, 1H), 6.43 (d, J=2.2 Hz, 1H), 3.89 (m, 6H) ppm.

### Step 2

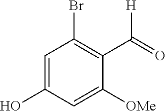 [SMILES: COc1cc(O)cc(Br)c1C=O]

### 2-bromo-4,6-dihydroxybenzaldehyde

To a solution of 2-bromo-4,6-dimethoxybenzaldehyde (50.0 mg, 204 mmol) in DCM (1.02 mL) at 0° C. was added boron tribromide (141 μL, 816 mmol). After 5 min at 0° C., the reaction mixture was stirred at room temperature for 48 hours then added to cold aqueous saturated NaHCO₃ solution. The aqueous mixture was extracted with DCM, dried over Na₂SO₄, filtered and the filtrate concentrated under reduced pressure to afford 2-bromo-4,6-dihydroxybenzaldehyde (49.2 mg, 204 mmol, 80% purity) as a yellow powder. ¹H NMR (400 MHz, CDCl₃) δ 12.40 (s, 1H), 10.11 (s, 1H), 6.70 (d, J=2.2 Hz, 1H), 6.35 (t, J=10.8 Hz, 1H) ppm.

### Step 3

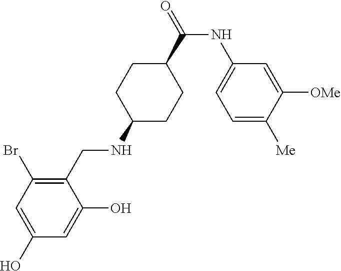 [SMILES: COc1cc(NC(=O)[C@H]2CC[C@@H](NCc3c(O)cc(O)cc3Br)CC2)ccc1C]

### (1s,4s)-4-((2-bromo-4,6-dihydroxybenzyl)amino)-N-(3-methoxy-4-methylphenyl)cyclohexane-1-carboxamide

To a solution of 2-bromo-6-hydroxybenzaldehyde (100 mg, 461 umol) and (1s,4s)-4-amino-N-(3-methoxy-4-methylphenyl)cyclohexanecarboxamide (127 mg, 484 umol) in DCE (3.07 mL) was added one drop of acetic acid. The mixture was stirred for 1 hour at 50° C. Then triethylamine (288 uL, 2.05 mmol) was added and the reaction mixture was stirred for 1.5 hours at 50° C. Sodium triacetoxyborohydride (292 mg, 1.38 mmol) was added in one portion and the resulting reaction mixture was stirred at 50° C. overnight. A saturated solution of sodium bicarbonate was added to reaction mixture and it was extracted 3 times with ethyl acetate. Organics were combined, washed with brine, dried over anhydrous sodium sulfate, filtered and the filtrate concentrated to dryness. The crude residue was purified by flash chromatography on silica gel (25 g column, from 0 to 100% 20/80 MeOH/DCM in DCM) to afford (1s,4s)-4-((2-bromo-4,6-dihydroxybenzyl)amino)-N-(3-methoxy-4-methylphenyl)cyclohexane-1-carboxamide (133 mg, 57% yield) as an off-white solid. LCMS ESI m/z: 463.3/465.5 (Br pattern) [M+H]⁺.

### Step 4

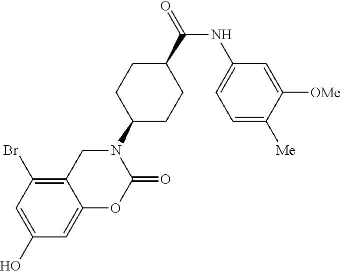 [SMILES: COc1cc(NC(=O)[C@H]2CC[C@@H](N3Cc4c(Br)cc(O)cc4OC3=O)CC2)ccc1C]

### (1s,4s)-4-(5-bromo-7-hydroxy-2-oxo-2H-benzo[e][1,3]oxazin-3(4H)-yl)-N-(3-methoxy-4-methylphenyl)cyclohexane-1-carboxamide

To a solution of (1s,4s)-4-((2-bromo-4,6-dihydroxybenzyl)amino)-N-(3-methoxy-4-methylphenyl)cyclohexane-1-carboxamide (133 mg, 287 umol) in THF (1.91 mL) at 0° C. was added triethylamine (60.3 uL, 431 umol) followed by triphosgene (34.8 mg, 115 umol). The resulting mixture was stirred at 0° C. for 5 min then at room temperature overnight. The reaction was quenched with 1M aqueous hydrochloric acid solution and extracted with EtOAc (×3). The organic extract was dried with anhydrous Na₂SO₄, filtered and the filtrate concentrated under reduced pressure to afford (1s,4s)-4-(5-bromo-7-hydroxy-2-oxo-2H-benzo[e][1,3]oxazin-3(4H)-yl)-N-(3-methoxy-4-methylphenyl)cyclohexane-1-carboxamide (140 mg, 100% yield). LCMS ESI m/z: 489.3/491.3 (Br pattern) [M+H]⁺.

### Step 5

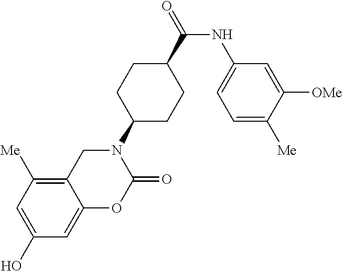 [SMILES: COc1cc(NC(=O)[C@H]2CC[C@@H](N3Cc4c(C)cc(O)cc4OC3=O)CC2)ccc1C]

### (1s,4s)-4-(7-hydroxy-5-methyl-2-oxo-2H-benzo[e][1,3]oxazin-3(4H)-yl)-N-(3-methoxy-4-methylphenyl)cyclohexane-1-carboxamide

A microwave vial was charged with 1,1′-bis(diphenylphosphino)ferrocene dichloropalladium (II) (12.5 mg, 17.1 mmol), cesium carbonate (338 mg, 1.03 mmol), methylboronic acid (25.6 mg, 428 mmol), (1s,4s)-4-(5-bromo-7-hydroxy-2-oxo-2H-benzo[e][1,3]oxazin-3(4H)-yl)-N-(3-methoxy-4-methylphenyl)cyclohexane-1-carboxamide (167 mg, 342 mmol), THF (1.71 mL) and water (171 mL). The vial was purged with nitrogen, capped and the reaction mixture heated to 100° C. After 50 mins, UPLC-MS confirmed the completed conversion to desired product. The reaction mixture was cooled to room temperature and diluted with EtOAc (15 mL). This mixture was washed with brine, dried over MgSO₄ and filtered. The filtrate was concentrated under reduced pressure. The crude residue was purified using reverse phase chromatography (12 g C18 column; eluent gradient 0 to 100% MeCN in 10 mM aqueous ammonium formate; 25 c.v.). Fractions containing desired product were combined and the MeCN was removed under reduced pressure. The resulting aqueous mixture was extracted with EtOAc (×4) and the organic separation dried over Na₂SO₄, filtered and the filtrate concentrated to afford (1s,4s)-4-(7-hydroxy-5-methyl-2-oxo-2H-benzo[e][1,3]oxazin-3(4H)-yl)-N-(3-methoxy-4-methylphenyl)cyclohexane-1-carboxamide (127 mg, 82% yield) as an off-white solid. LCMS ESI m/z: 425.3 [M+H]⁺. ¹H NMR (400 MHz, d₆-DMSO) δ 9.68 (s, 1H), 9.56 (s, 1H), 7.26 (d, J=1.6 Hz, 1H), 7.09 (dd, J=8.0, 1.8 Hz, 1H), 6.99 (d, J=8.2 Hz, 1H), 6.38 (d, J=1.7 Hz, 1H), 6.17 (d, J=2.2 Hz, 1H), 4.17 (s, 2H), 4.11-3.95 (m, 1H), 3.73 (s, 3H), 2.63 (s, 1H), 2.11 (d, J=11.6 Hz, 5H), 2.06 (s, 3H), 1.98 (dd, J=16.6, 7.7 Hz, 2H), 1.64 (t, J=13.4 Hz, 2H), 1.54 (d, J=11.7 Hz, 2H) ppm.

### Example 117

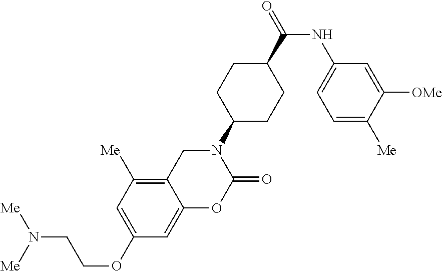 [SMILES: COc1cc(NC(=O)[C@H]2CC[C@@H](N3Cc4c(C)cc(OCCN(C)C)cc4OC3=O)CC2)ccc1C]

### (1s,4s)-4-(7-(2-(dimethylamino)ethoxy)-5-methyl-2-oxo-2H-benzo[e][1,3]oxazin-3(4H)-yl)-N-(3-methoxy-4-methylphenyl)cyclohexane-1-carboxamide

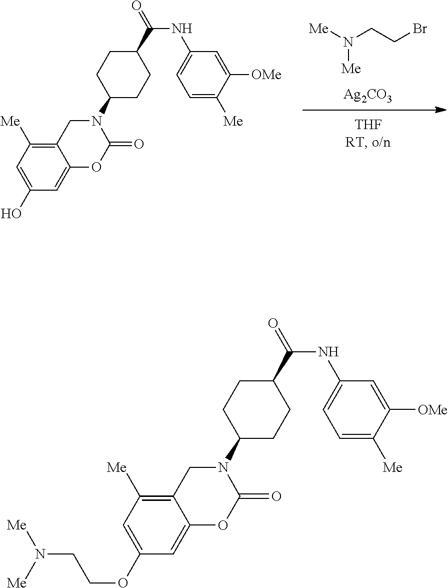

To a microwave tube was added 2-bromo-N,N-dimethylethanamine hydrobromide (10.1 mL, 94.2 mmol), (1s,4s)-4-(7-hydroxy-5-methyl-2-oxo-2H-benzo[e][1,3]oxazin-3(4H)-yl)-N-(3-methoxy-4-methylphenyl)cyclohexane-1-carboxamide (20.0 mg, 47.1 mmol) and THF (471 mL). Then to the resulting mixture at room temperature was added silver carbonate (79.5 mg, 283 mmol). After stirring overnight at room temperature EtOAc was added to the reaction mixture and sonicated to suspend the precipitate in solution. The resulting mixture was filtered through a pad of Celite and the filtrate concentrated under reduced pressure. The crude residue was purified using reverse phase column chromatography (6 g C18 column eluted with gradient of 0 to 80% MeCN in 10 mM ammonium formate in water; 15 c.v.). Fractions containing product were combined and concentrated to remove MeCN. The resulting aqueous mixture was frozen and water removed by lyophilizer to afford (1s,4s)-4-(7-(2-(dimethylamino)ethoxy)-5-methyl-2-oxo-2H-benzo[e][1,3]oxazin-3(4H)-yl)-N-(3-methoxy-4-methylphenyl)cyclohexane-1-carboxamide (1.2 mg, 5.2% yield) as a white solid. LCMS ESI m/z: 496.3 [M+H]⁺. ¹H NMR (400 MHz, d₆-DMSO) δ 9.71-9.68 (m, 1H), 7.27 (s, 1H), 7.11-7.09 (m, 1H), 7.09-7.08 (m, 1H), 7.01 (s, 1H), 7.00-6.98 (m, 1H), 6.57 (s, 1H), 6.42 (s, 1H), 4.22 (s, 2H), 3.98 (s, 2H), 3.73 (s, 3H), 2.65 (s, 4H), 2.56 (s, 4H), 2.31 (s, 2H), 2.17 (s, 7H), 2.15 (s, 3H), 2.11-2.08 (m, 1H), 2.06 (s, 3H), 2.04-1.98 (m, 1H), 1.70-1.60 (m, 1H), 1.60-1.51 (m, 1H), 1.21 (s, 2H) ppm.

### Example 118

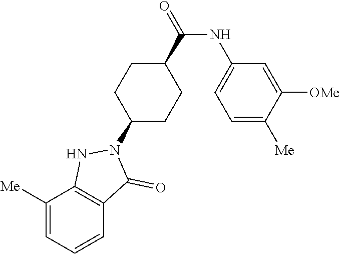 [SMILES: COc1cc(NC(=O)[C@H]2CC[C@@H](n3[nH]c4c(C)cccc4c3=O)CC2)ccc1C]

### (1s,4s)-N-(3-Methoxy-4-methylphenyl)-4-(7-methyl-3-oxo-1,3-dihydro-2H-indazol-2-yl)cyclohexane-1-carboxamide

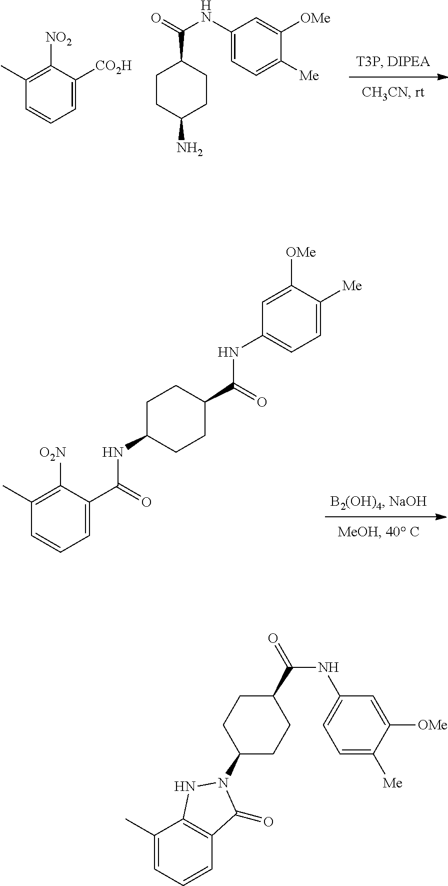

### Step 1

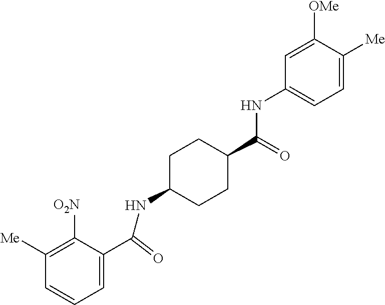 [SMILES: COc1cc(NC(=O)[C@H]2CC[C@@H](NC(=O)c3cccc(C)c3[N+](=O)[O-])CC2)ccc1C]

### N-((1s,4s)-4-((3-Methoxy-4-methylphenyl)carbamoyl)cyclohexyl)-3-methyl-2-nitrobenzamide

To a solution of (1s,4s)-4-amino-N-(3-methoxy-4-methylphenyl)cyclohexane-1-carboxamide (145 mg, 552 μmol) and 3-methyl-2-nitrobenzoic acid (100 mg, 552 μmol) in ACN (2.0 mL) was added N,N-diisopropylethylamine (213 μL, 1.21 mmol) and 1-propylphosphonic acid cyclic anhydride (T3P) (361 μL, 607 μmol). The reaction mixture was stirred at room temperature for 4 hours. The reaction mixture was evaporated in vacuo. The residue was purified by regular phase chromatography (silica gel 25 g, 0 to 20% MeOH in DCM) to obtain N-((1s,4s)-4-((3-methoxy-4-methylphenyl)carbamoyl)cyclohexyl)-3-methyl-2-nitrobenzamide as a yellow solid (186 mg, 79% Yield); LCMS ESI m/z: 426.4 [M+H]⁺.

### Step 2

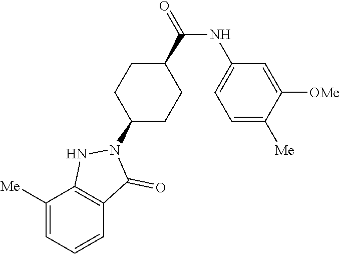 [SMILES: COc1cc(NC(=O)[C@H]2CC[C@@H](n3[nH]c4c(C)cccc4c3=O)CC2)ccc1C]

### (1s,4s)-N-(3-Methoxy-4-methylphenyl)-4-(7-methyl-3-oxo-1,3-dihydro-2H-indazol-2-yl)cyclohexane-1-carboxamide

To a solution of N-((1s,4s)-4-((3-methoxy-4-methylphenyl)carbamoyl)cyclohexyl)-3-methyl-2-nitrobenzamide (166 mg, 390 μmol) in MeOH (5.00 mL) was added tetrahydroxydiboron (180 mg, 1.95 mmol). The reaction mixture was cooled to 0° C. and NaOH [0.8 M in MeOH] (4.88 mL, 3.90 mmol) was added dropwise. The reaction mixture was warmed to 40° C. and stirred overnight. The reaction mixture was evaporated in vacuo and the residue was purified by regular phase chromatography (silica gel 25 g, 0 to 20% MeOH in DCM) to obtain (1s,4s)-N-(3-Methoxy-4-methylphenyl)-4-(7-methyl-3-oxo-1,3-dihydro-2H-indazol-2-yl)cyclohexane-1-carboxamide (65.5 mg, 43% Yield); LCMS ESI m/z: 394.2 [M+H]⁺; ¹H NMR (400 MHz, DMSO-d₆) δ 9.71 (s, 1H), 9.54 (s, 1H), 7.44 (d, J=7.7 Hz, 1H), 7.37 (d, J=1.5 Hz, 1H), 7.28 (d, J=7.1 Hz, 1H), 7.13 (dd, J=8.0, 1.7 Hz, 1H), 7.01 (t, J=7.6 Hz, 2H), 4.28 (t, J=11.0 Hz, 1H), 3.76 (s, 3H), 2.68 (s, 1H), 2.34 (s, 3H), 2.17 (t, J=11.4 Hz, 4H), 2.08 (s, 3H), 1.72 (t, J=11.6 Hz, 2H), 1.62 (d, J=9.5 Hz, 2H).

### Example 119

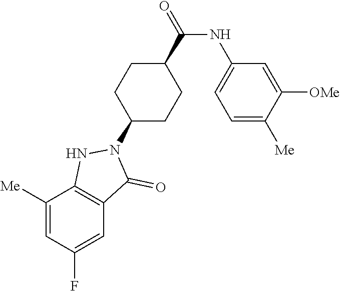 [SMILES: COc1cc(NC(=O)[C@H]2CC[C@@H](n3[nH]c4c(C)cc(F)cc4c3=O)CC2)ccc1C]

### (1s,4s)-4-(5-Fluoro-7-methyl-3-oxo-1,3-dihydro-2H-indazol-2-yl)-N-(3-methoxy-4-methylphenyl)cyclohexane-1-carboxamide

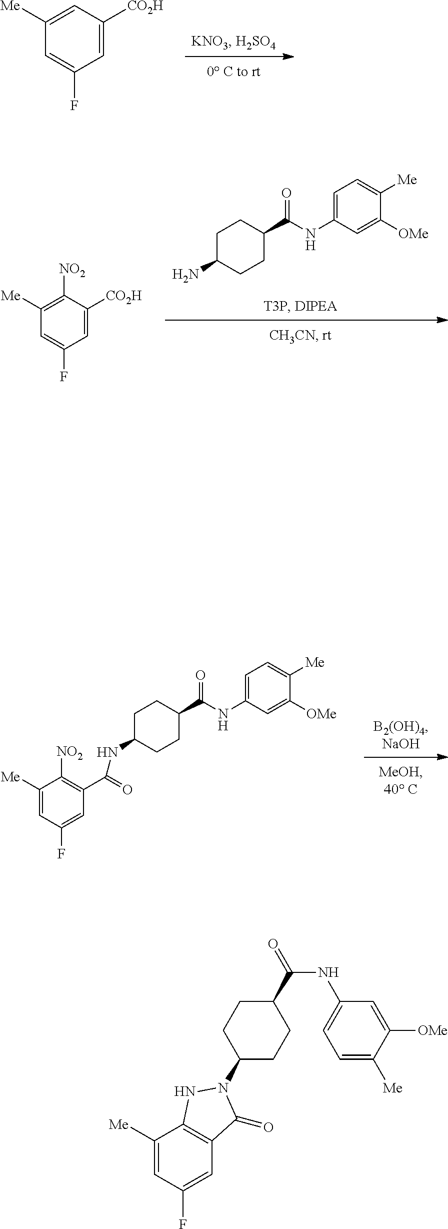

### Step 1

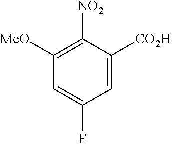 [SMILES: COc1cc(F)cc(C(=O)O)c1[N+](=O)[O-]]

### 5-Fluoro-3-methyl-2-nitrobenzoic Acid

To a solution of 3-fluoro-5-methylbenzoic acid (4.00 g, 26.0 mmol) in H₂SO₄ (30 mL) was added potassium nitrate (2.92 g, 28.5 mmol) at 0° C. The reaction mixture was warmed to room temperature over 90 min. Crushed ice was added to the reaction mixture and the solution was stirred for 30 min leading to formation of white solid. The precipitated solid was vacuum filtered to obtained 5-fluoro-3-methyl-2-nitrobenzoic acid (4.13 g, 80% Yield); LCMS ESI m/z: 198.0 [M−H]⁺.

### Step 2

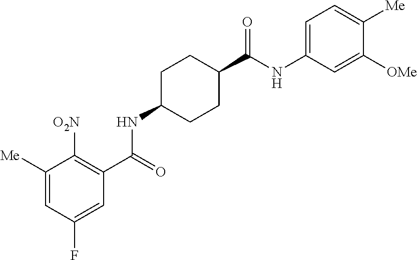 [SMILES: COc1cc(NC(=O)[C@H]2CC[C@@H](NC(=O)c3cc(F)cc(C)c3[N+](=O)[O-])CC2)ccc1C]

### 5-Fluoro-N-((1s,4s)-4-((3-methoxy-4-methylphenyl)carbamoyl)cyclohexyl)-3-methyl-2-nitrobenzamide

To a solution of (1s,4s)-4-amino-N-(3-methoxy-4-methylphenyl)cyclohexane-1-carboxamide (311 mg, 829 μmol) and 5-fluoro-3-methyl-2-nitrobenzoic acid (150 mg, 753 μmol) in ACN (10 mL) was added N,N-diisopropylethylamine (290 μL, 1.66 mmol) and 1-propylphosphonic acid cyclic anhydride (493 μL, 829 μmol). The reaction mixture was stirred at room temperature for 4 h. The reaction mixture was evaporated on vacuo and the residue was purified by regular phase chromatography (silica gel 25 g, 0 to 20% MeOH in DCM) to obtain 5-fluoro-N-((1s,4s)-4-((3-methoxy-4-methylphenyl) carbamoyl)cyclohexyl)-3-methyl-2-nitrobenzamide as a white solid (227 mg, 68% Yield). LCMS ESI m/z: 444.2 [M+H]⁺.

### Step 3

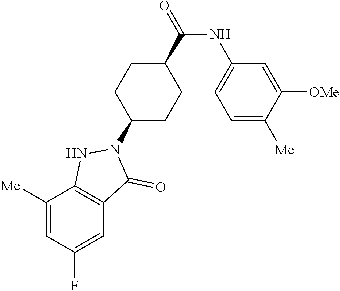 [SMILES: COc1cc(NC(=O)[C@H]2CC[C@@H](n3[nH]c4c(C)cc(F)cc4c3=O)CC2)ccc1C]

### (1s,4s)-4-(5-Fluoro-7-methyl-3-oxo-1,3-dihydro-2H-indazol-2-yl)-N-(3-methoxy-4-methylphenyl)cyclohexane-1-carboxamide

To a solution of 5-fluoro-N-((1s,4s)-4-((3-methoxy-4-methylphenyl)carbamoyl)cyclohexyl)-3-methyl-2-nitrobenzamide (182 mg, 410 μmol) in MeOH (5.00 mL) was added tetrahydroxydiboron (190 mg, 2.05 mmol). The reaction mixture was cooled to 0° C. and NaOH [0.8 M in MeOH] (5.13 mL, 4.10 mmol) was added dropwise. The reaction mixture was warmed to 40° C. and stirred overnight. The reaction mixture was evaporated in vacuo and the residue was purified by regular phase chromatography (Silica gel 25 g, 0 to 20% MeOH in DCM) to obtain (1s,4s)-4-(5-fluoro-7-methyl-3-oxo-1,3-dihydro-2H-indazol-2-yl)-N-(3-methoxy-4-methylphenyl)cyclohexane-1-carboxamide as a white solid (92.4 mg, 55% Yield); LCMS ESI m/z: 412.2 [M+H]⁺; ¹H NMR (400 MHz, DMSO-d₆) δ 9.70 (s, 1H), 9.58 (s, 1H), 7.36 (d, J=1.5 Hz, 1H), 7.21 (t, J=8.4 Hz, 2H), 7.13 (dd, J=8.0, 1.7 Hz, 1H), 7.02 (d, J=8.2 Hz, 1H), 4.29 (t, J=10.9 Hz, 1H), 3.76 (s, 3H), 2.68 (s, 1H), 2.35 (s, 3H), 2.17 (t, J=11.0 Hz, 4H), 2.08 (d, J=4.5 Hz, 3H), 1.72 (t, J=11.5 Hz, 2H), 1.65 (dd, J=12.8, 7.8 Hz, 2H).

### Example 120

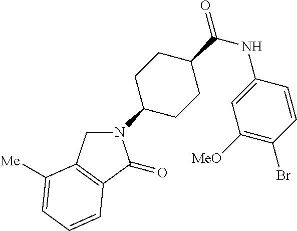 [SMILES: COc1cc(NC(=O)[C@H]2CC[C@@H](N3Cc4c(C)cccc4C3=O)CC2)ccc1Br]

### (1s,4s)-N-(4-Bromo-3-methoxyphenyl)-4-(4-methyl-1-oxoisoindolin-2-yl)cyclohexane-1-carboxamide

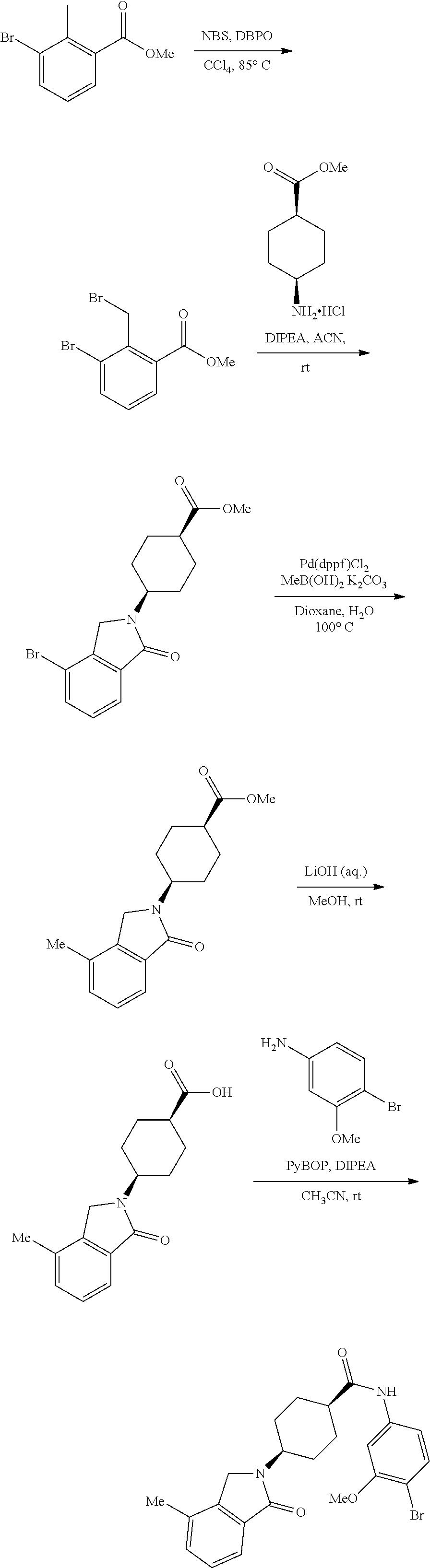

### Step 1

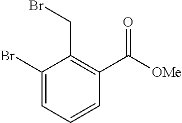 [SMILES: COC(=O)c1cccc(Br)c1CBr]

### Methyl 3-bromo-2-(bromomethyl)benzoate

To a solution of methyl 3-bromo-2-methylbenzoate (10.0 g, 43.7 mmol) in CCl₄ (200 mL) was added benzoyl peroxide (540 mg, 2.18 mmol) and N-bromosuccinimide (8.63 g, 48.0 mmol). The reaction mixture was refluxed/heated at 85° C. for 16 hours. The reaction mixture was cooled down and evaporated in vacuo. The residue was purified by regular phase chromatography (silica gel 100 g, 0 to 10% EtOAc in Heptanes) to obtain methyl 3-bromo-2-(bromomethyl)benzoate (10.9 g, 81% Yield); ¹H NMR (400 MHz, CDCl₃) δ 7.89 (d, J=6.2 Hz, 1H), 7.76 (d, J=6.5 Hz, 1H), 7.32-7.13 (m, 1H), 5.13 (s, 2H), 3.96 (s, 3H).

### Step 2

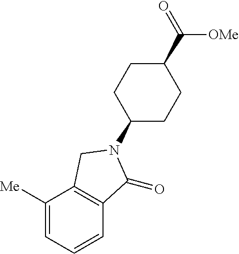 [SMILES: COC(=O)[C@H]1CC[C@@H](N2Cc3c(C)cccc3C2=O)CC1]

### Methyl (1s,4s)-4-(4-bromo-1-oxoisoindolin-2-yl)cyclohexane-1-carboxylate

To a solution of methyl 3-bromo-2-methyl-5-nitrobenzoate (1.76 g, 5.71 mmol) in ACN (50.0 mL) was added cis-4-amino-cyclohexylcarboxylic acid methyl ester hydrochloride (1.03 g, 6.29 mmol) and N,N-diisopropylethylamine (2.20 mL, 12.6 mmol). The reaction mixture was stirred at room temperature for 4 hours. The reaction mixture was evaporated in vacuo and the residue was purified by regular phase chromatography (silica gel 100 g, 0 to 50% EtOAc in Heptanes) to obtain methyl (1s,4s)-4-(4-bromo-1-oxoisoindolin-2-yl)cyclohexane-1-carboxylate (1.26 g, 63% Yield); ¹H NMR (400 MHz, CDCl₃) δ 7.73 (d, J=7.5 Hz, 1H), 7.62-7.48 (m, 1H), 7.30 (t, J=7.7 Hz, 1H), 4.35-4.07 (m, 3H), 3.71 (s, 3H), 2.68 (s, 1H), 2.25 (d, J=8.0 Hz, 2H), 1.84-1.55 (m, 6H).

### Step 3

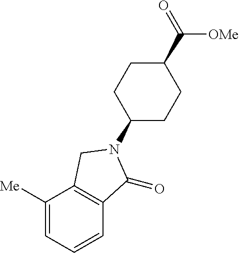 [SMILES: COC(=O)[C@H]1CC[C@@H](N2Cc3c(C)cccc3C2=O)CC1]

### Methyl (1s,4s)-4-(4-methyl-1-oxoisoindolin-2-yl)cyclohexane-1-carboxylate

To a solution of methyl (1s,4s)-4-(4-bromo-1-oxoisoindolin-2-yl)cyclohexane-1-carboxylate (1.26 g, 3.58 mmol) in dioxane (25.0 mL) and water (5.00 mL) was added potassium carbonate (1.51 g, 10.7 mmol), methylboronic acid (257 mg, 4.29 mmol) and 1,1′-bis(diphenylphosphino)ferrocene dichloropalladium (II) (276 mg, 358 μmol). The reaction mixture was heated at 100° C. for 2 hours. The reaction mixture was cooled down and evaporated in vacuo. The residue was purified by regular phase chromatography (silica gel 50 g, 0 to 50% EtOAc in Heptanes) to obtain methyl (1s,4s)-4-(4-methyl-1-oxoisoindolin-2-yl)cyclohexane-1-carboxylate (810 mg, 79% Yield); LCMS ESI m/z: 288.3 [M+H]⁺.

### Step 4

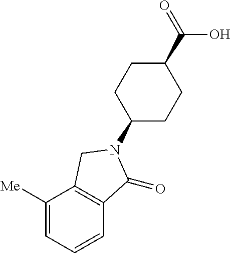 [SMILES: Cc1cccc2c1CN([C@H]1CC[C@@H](C(=O)O)CC1)C2=O]

### (1s,4s)-4-(4-Methyl-1-oxoisoindolin-2-yl)cyclohexane-1-carboxylic acid

To a solution of methyl (1s,4s)-4-(4-methyl-1-oxoisoindolin-2-yl)cyclohexane-1-carboxylate (810 mg, 2.82 mmol) in MeOH (10.0 mL) was added 1M KOH (2.82 mL, 2.82 mmol). The reaction mixture was stirred for 8 hours at room temperature. The reaction mixture was quenched with 1M HCl (bringing to pH 7) and evaporated in vacuo. The residue was purified by regular phase chromatography (silica gel 25 g, 0 to 20% MeOH in DCM) to obtain (1s,4s)-4-(4-methyl-1-oxoisoindolin-2-yl)cyclohexane-1-carboxylic acid (700 mg, 91% Yield); LCMS ESI m/z: 274.2 [M+H]⁺.

### Step 5

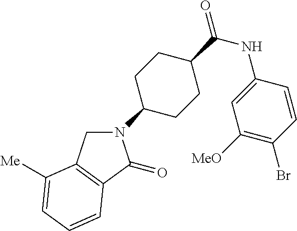 [SMILES: COc1cc(NC(=O)[C@H]2CC[C@@H](N3Cc4c(C)cccc4C3=O)CC2)ccc1Br]

### (1s,4s)-N-(4-Bromo-3-methoxyphenyl)-4-(4-methyl-1-oxoisoindolin-2-yl)cyclohexane-1-carboxamide

To a solution of (1s,4s)-4-(4-methyl-1-oxoisoindolin-2-yl)cyclohexane-1-carboxylic acid (350 mg, 1.28 mmol) in ACN (10.0 mL) was added N,N-diisopropylethylamine (986 UL, 5.63 mmol), 4-bromo-3-methoxyaniline (290 mg, 1.41 mmol) and PyBOP (1.50 g, 2.82 mmol). The reaction mixture was stirred at room temperature for 24 hours. The reaction mixture was evaporated in vacuo and the residue was purified by regular phase chromatography (silica gel 25 g, 0 to 20% MeOH in DCM) to obtain (1s,4s)-N-(4-Bromo-3-methoxyphenyl)-4-(4-methyl-1-oxoisoindolin-2-yl)cyclohexane-1-carboxamide (299 mg, 51% Yield) as a white solid; LCMS ESI m/z: 457.2 and 459.1 [M]⁺ and [M+2H]⁺; ¹H NMR (400 MHz, DMSO-d₆) δ 9.95 (s, 1H), 7.52 (d, J=2.2 Hz, 1H), 7.47 (dd, J=10.0, 6.0 Hz, 2H), 7.38 (dd, J=7.2, 5.5 Hz, 2H), 7.21 (dd, J=8.6, 2.3 Hz, 1H), 4.38 (s, 2H), 4.12 (ddd, J=11.3, 7.8, 3.8 Hz, 1H), 3.82 (s, 3H), 2.70 (s, 1H), 2.34 (s, 3H), 2.13 (d, J=12.4 Hz, 2H), 2.05-1.89 (m, 2H), 1.85-1.67 (m, 2H), 1.62 (dd, J=8.5, 3.8 Hz, 2H).

### Example 121

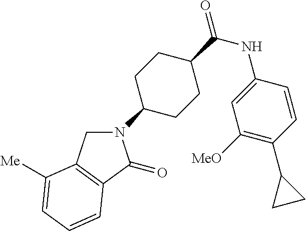 [SMILES: COc1cc(NC(=O)[C@H]2CC[C@@H](N3Cc4c(C)cccc4C3=O)CC2)ccc1C1CC1]

### (1s,4s)-N-(4-Cyclopropyl-3-methoxyphenyl)-4-(4-methyl-1-oxoisoindolin-2-yl)cyclohexane-1-carboxamide

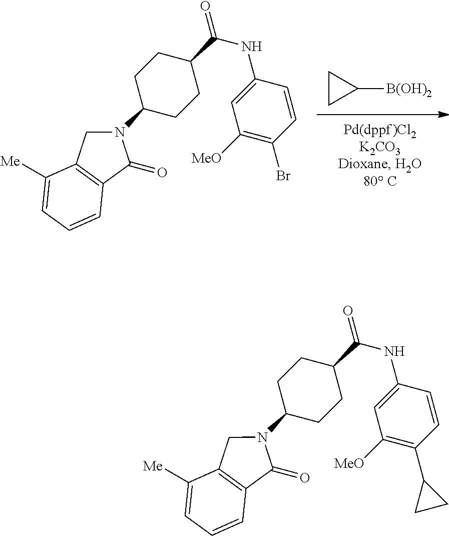

### Step 1

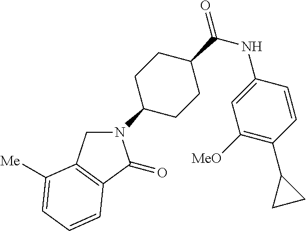 [SMILES: COc1cc(NC(=O)[C@H]2CC[C@@H](N3Cc4c(C)cccc4C3=O)CC2)ccc1C1CC1]

### (1s,4s)-N-(4-Cyclopropyl-3-methoxyphenyl)-4-(4-methyl-1-oxoisoindolin-2-yl)cyclohexane-1-carboxamide

To a solution of (1s,4s)-N-(4-bromo-3-methoxyphenyl)-4-(4-methyl-1-oxoisoindolin-2-yl)cyclohexane-1-carboxamide (50.0 mg, 109 μmol) in dioxane (2.5 mL) and water (500 μL) was added cyclopropylboronic acid (12.5 mg, 142 μmol), potassium carbonate (45.3 mg, 328 μmol) and 1,1′-bis(diphenylphosphino)ferrocene dichloropalladium (II) (12.2 mg, 16.4 μmol). The reaction mixture was stirred at 100° C. for 3 hours. The reaction mixture was evaporated in vacuo and the residue was purified by regular phase chromatography (silica gel 25 g, 0 to 20% MeOH in DCM) to obtain (1s,4s)-N-(4-cyclopropyl-3-methoxyphenyl)-4-(4-methyl-1-oxoisoindolin-2-yl)cyclohexane-1-carboxamide as a white solid (21.1 mg, 46% Yield); LCMS ESI m/z: 419.2 [M+H]⁺; ¹H NMR (400 MHz, DMSO-d₆) δ 9.73 (s, 1H), 7.56-7.43 (m, 1H), 7.37 (dd, J=6.5, 3.1 Hz, 2H), 7.34 (d, J=1.8 Hz, 1H), 7.12 (dd, J=8.3, 1.8 Hz, 1H), 6.74 (d, J=8.3 Hz, 1H), 4.38 (s, 2H), 4.11 (dd, J=13.1, 8.1 Hz, 1H), 3.78 (s, 3H), 3.17 (d, J=5.0 Hz, 1H), 2.67 (s, 1H), 2.33 (s, 3H), 2.13 (d, J=12.2 Hz, 2H), 1.99 (dd, J=9.3, 4.1 Hz, 2H), 1.73 (dd, J=17.2, 8.8 Hz, 2H), 1.61 (dd, J=8.5, 3.7 Hz, 2H), 0.90-0.76 (m, 2H), 0.65-0.44 (m, 2H).

### Example 122

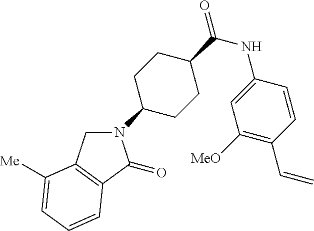 [SMILES: C=Cc1ccc(NC(=O)[C@H]2CC[C@@H](N3Cc4c(C)cccc4C3=O)CC2)cc1OC]

### (1s,4s)-N-(3-Methoxy-4-vinylphenyl)-4-(4-methyl-1-oxoisoindolin-2-yl)cyclohexane-1-carboxamide

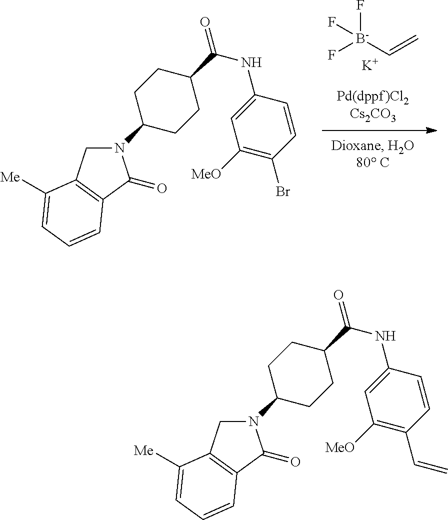

### Step 1

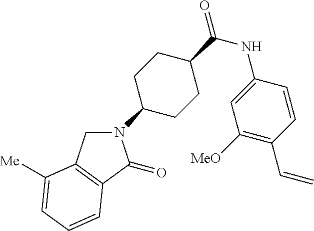 [SMILES: C=Cc1ccc(NC(=O)[C@H]2CC[C@@H](N3Cc4c(C)cccc4C3=O)CC2)cc1OC]

### (1s,4s)-N-(3-Methoxy-4-vinylphenyl)-4-(4-methyl-1-oxoisoindolin-2-yl)cyclohexane-1-carboxamide

To a solution of (1s,4s)-N-(4-bromo-3-methoxyphenyl)-4-(4-methyl-1-oxoisoindolin-2-yl)cyclohexane-1-carboxamide (100 mg, 219 μmol) in dioxane (2 mL) and water (0.2 mL) was added 1,1′-bis(diphenylphosphino)ferrocene dichloropalladium (II) (16.3 mg, 21.9 μmol), cesium carbonate (218 mg, 656 μmol) and potassium vinyltrifluoroborate (33.2 mg, 241 μmol). The tube was charged with N₂ and sealed. The reaction mixture was heated at 65° C. for 4 hours. The reaction mixture was concentrated in vacuo and the crude residue was purified by reverse phase chromatography (C18 25 g, 0 to 100% ACN in ammonium formate) to obtain (1s,4s)-N-(3-methoxy-4-vinylphenyl)-4-(4-methyl-1-oxoisoindolin-2-yl)cyclohexane-1-carboxamide (46.0 mg, 52% Yield); LCMS ESI m/z: 405.2 [M+H]⁺; ¹H NMR (400 MHz, DMSO-d₆) δ 9.88 (s, 1H), 7.48 (dd, J=8.8, 3.6 Hz, 1H), 7.46-7.40 (m, 2H), 7.38 (dd, J=7.3, 5.5 Hz, 2H), 7.23 (dd, J=8.4, 1.6 Hz, 1H), 6.87 (dd, J=17.8, 11.2 Hz, 1H), 5.69 (dd, J=17.8, 1.5 Hz, 1H), 5.14 (dd, J=11.3, 1.4 Hz, 1H), 4.39 (s, 2H), 4.23-4.03 (m, 1H), 3.79 (s, 3H), 2.70 (s, 1H), 2.34 (s, 3H), 2.14 (d, J=12.3 Hz, 2H), 2.08-1.89 (m, 2H), 1.74 (dd, J=17.3, 8.6 Hz, 2H), 1.68-1.52 (m, 2H).

### Example 123

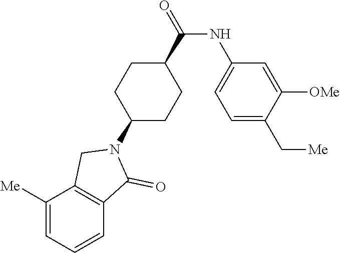 [SMILES: CCc1ccc(NC(=O)[C@H]2CC[C@@H](N3Cc4c(C)cccc4C3=O)CC2)cc1OC]

### (1s,4s)-N-(4-Ethyl-3-methoxyphenyl)-4-(4-methyl-1-oxoisoindolin-2-yl)cyclohexane-1-carboxamide

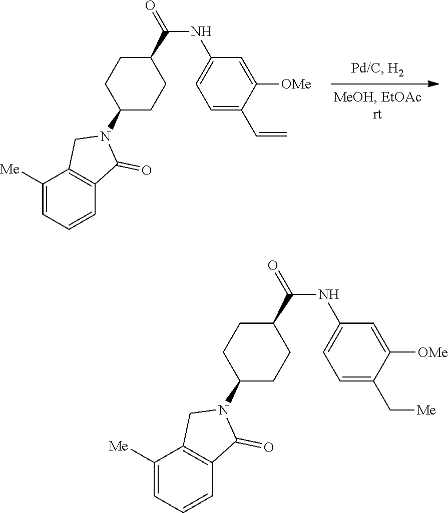 [SMILES: C=Cc1ccc(NC(=O)[C@H]2CC[C@@H](N3Cc4c(C)cccc4C3=O)CC2)cc1OC ; CCc1ccc(NC(=O)[C@H]2CC[C@@H](N3Cc4c(C)cccc4C3=O)CC2)cc1OC]

### Step 1

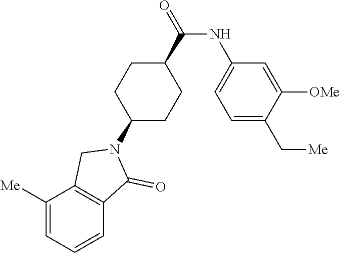 [SMILES: CCc1ccc(NC(=O)[C@H]2CC[C@@H](N3Cc4c(C)cccc4C3=O)CC2)cc1OC]

### (1s,4s)-N-(4-Ethyl-3-methoxyphenyl)-4-(4-methyl-1-oxoisoindolin-2-yl)cyclohexane-1-carboxamide

To a solution of (1s,4s)-N-(3-methoxy-4-vinylphenyl)-4-(4-methyl-1-oxoisoindolin-2-yl)cyclohexane-1-carboxamide (50.0 mg, 124 μmol) in MeOH (1.00 mL) and EtOAc (1.00 mL) was added Palladium/C (26.3 mg, 24.7 μmol). The reaction mixture was charged with H₂ and stirred overnight at room temperature. The reaction mixture was filtered through Celite, concentrated in vacuo and the crude residue was purified by reverse phase chromatography (C18 25 g, 0 to 100% ACN in ammonium formate) to obtain (1s,4s)-N-(4-ethyl-3-methoxyphenyl)-4-(4-methyl-1-oxoisoindolin-2-yl)cyclohexane-1-carboxamide (40.6 mg, 81% Yield); LCMS ESI m/z: 407.4 [M+H]⁺; ¹H NMR (400 MHz, DMSO-d₆) δ 9.74 (s, 1H), 7.48 (dd, J=5.2, 3.3 Hz, 1H), 7.42-7.28 (m, 3H), 7.15 (dd, J=8.1, 1.7 Hz, 1H), 7.02 (d, J=8.1 Hz, 1H), 4.38 (s, 2H), 4.11 (dd, J=13.2, 9.7 Hz, 1H), 3.75 (s, 3H), 3.34 (s, 1H), 2.67 (s, 1H), 2.33 (s, 3H), 2.13 (d, J=12.5 Hz, 2H), 2.05-1.88 (m, 2H), 1.72 (dd, J=17.3, 8.6 Hz, 2H), 1.60 (dd, J=8.5, 3.6 Hz, 2H), 1.29 (s, 1H), 1.08 (t, J=7.5 Hz, 3H).

### Example 124

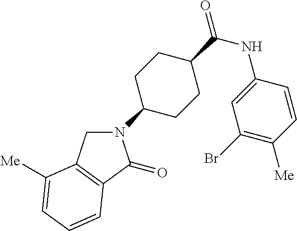 [SMILES: Cc1ccc(NC(=O)[C@H]2CC[C@@H](N3Cc4c(C)cccc4C3=O)CC2)cc1Br]

### (1s,4s)-N-(3-Bromo-4-methylphenyl)-4-(4-methyl-1-oxoisoindolin-2-yl)cyclohexane-1-carboxamide

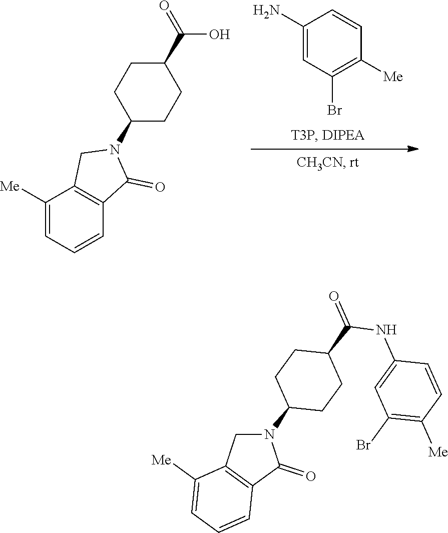 [SMILES: Cc1cccc2c1CN([C@H]1CC[C@@H](C(=O)O)CC1)C2=O ; Cc1ccc(N)cc1Br ; Cc1ccc(NC(=O)[C@H]2CC[C@@H](N3Cc4c(C)cccc4C3=O)CC2)cc1Br]

### Step 1

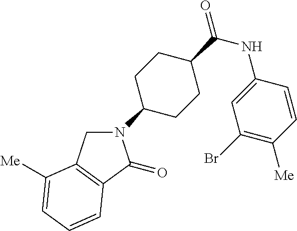 [SMILES: Cc1ccc(NC(=O)[C@H]2CC[C@@H](N3Cc4c(C)cccc4C3=O)CC2)cc1Br]

### (1s,4s)-N-(3-Bromo-4-methylphenyl)-4-(4-methyl-1-oxoisoindolin-2-yl)cyclohexane-1-carboxamide

To a solution of (1s,4s)-4-(4-methyl-1-oxoisoindolin-2-yl)cyclohexane-1-carboxylic acid (100 mg, 366 μmol) in ACN (6.00 mL) was added N,N-diisopropylethylamine (141 μL, 805 μmol), 3-bromo-4-methylaniline (76.2 mg, 410 μmol) and 1-propylphosphonic acid cyclic anhydride 50% w in EtOAc (240 μL, 402 μmol). The reaction mixture was stirred for 24 hours at room temperature. The reaction mixture was concentrated in vacuo and the crude residue was purified by reverse phase chromatography (C18 25 g, 0 to 100% ACN in Ammonium formate) to obtain (1s,4s)-N-(3-bromo-4-methylphenyl)-4-(4-methyl-1-oxoisoindolin-2-yl)cyclohexane-1-carboxamide (77.0 mg, 48% Yield); LCMS ESI m/z: 441.2 and 443.2 [M]⁺ and [M+H]⁺; ¹H NMR (400 MHz, DMSO-d₆) δ 9.90 (s, 1H), 8.06 (d, J=2.1 Hz, 1H), 7.50-7.40 (m, 2H), 7.40-7.33 (m, 2H), 7.27 (d, J=8.4 Hz, 1H), 4.38 (s, 2H), 4.11 (ddd, J=11.4, 7.8, 3.8 Hz, 1H), 2.68 (s, 1H), 2.34 (s, 3H), 2.28 (s, 3H), 2.12 (d, J=12.6 Hz, 2H), 1.99 (dt, J=21.5, 7.5 Hz, 2H), 1.84-1.66 (m, 2H), 1.61 (dd, J=8.6, 3.7 Hz, 2H).

### Example 125

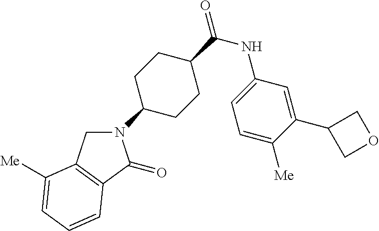 [SMILES: Cc1ccc(NC(=O)[C@H]2CC[C@@H](N3Cc4c(C)cccc4C3=O)CC2)cc1C1COC1]

### (1s,4s)-4-(4-Methyl-1-oxoisoindolin-2-yl)-N-(4-methyl-3-(oxetan-3-yl)phenyl)cyclohexane-1-carboxamide

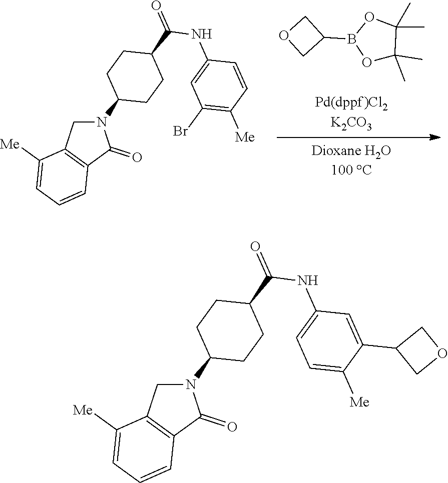 [SMILES: Cc1ccc(NC(=O)[C@H]2CC[C@@H](N3Cc4c(C)cccc4C3=O)CC2)cc1Br ; CC1(C)OB(C2COC2)OC1(C)C ; Cc1ccc(NC(=O)[C@H]2CC[C@@H](N3Cc4c(C)cccc4C3=O)CC2)cc1C1COC1]

### Step 1

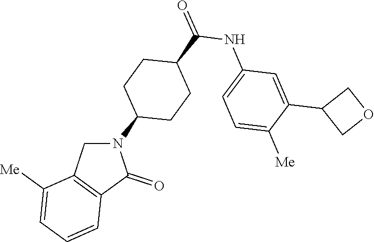 [SMILES: Cc1ccc(NC(=O)[C@H]2CC[C@@H](N3Cc4c(C)cccc4C3=O)CC2)cc1C1COC1]

### (1s,4s)-4-(4-Methyl-1-oxoisoindolin-2-yl)-N-(4-methyl-3-(oxetan-3-yl)phenyl)cyclohexane-1-carboxamide

To a solution of (1s,4s)-N-(3-bromo-4-methylphenyl)-4-(4-methyl-1-oxoisoindolin-2-yl)cyclohexane-1-carboxamide (55.0 mg, 125 μmol) in dioxane (3.00 mL) and water (600 μL) was added 1,1′-bis(diphenylphosphino)ferrocene dichloropalladium (II) (14.0 mg, 18.7 μmol), 4,4,5,5-tetramethyl-2-(oxetan-3-yl)-1,3,2-dioxaborolane (26.6 mg, 137 μmol) and potassium acetate (37.1 mg, 374 μmol). The reaction mixture was stirred at 100° C. for 2 hours. The reaction mixture was purified using reverse phase chromatography (C18 25 g, 0 to 100% ACN in ammonium bicarbonate) to obtain (1s,4s)-4-(4-methyl-1-oxoisoindolin-2-yl)-N-(4-methyl-3-(oxetan-3-yl)phenyl)cyclohexane-1-carboxamide (2.03 mg, 3.9% Yield); LCMS ESI m/z: 419.3 [M+H]⁺; ¹H NMR (400 MHz, DMSO-d₆) δ 9.74 (d, J=21.6 Hz, 1H), 7.66 (t, J=5.2 Hz, 1H), 7.55 (dd, J=25.8, 3.5 Hz, 1H), 7.48 (d, J=5.3 Hz, 1H), 7.38 (dd, J=7.4, 5.4 Hz, 2H), 7.08 (t, J=8.4 Hz, 1H), 4.94 (dd, J=8.4, 5.7 Hz, 2H), 4.64 (dd, J=7.2, 5.8 Hz, 2H), 4.45 (q, J=7.9 Hz, 1H), 4.39 (s, 2H), 4.12 (t, J=11.2 Hz, 1H), 2.69 (s, 1H), 2.29 (d, J=36.5 Hz, 3H), 2.14 (d, J=12.8 Hz, 2H), 2.06 (d, J=9.3 Hz, 3H), 1.99 (dd, J=16.5, 7.1 Hz, 2H), 1.74 (dd, J=17.0, 8.6 Hz, 2H), 1.66-1.53 (m, 2H).

### Example 126

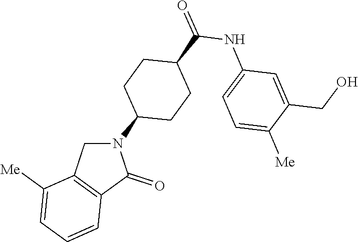 [SMILES: Cc1ccc(NC(=O)[C@H]2CC[C@@H](N3Cc4c(C)cccc4C3=O)CC2)cc1CO]

### (1s,4s)-N-(3-(Hydroxymethyl)-4-methylphenyl)-4-(4-methyl-1-oxoisoindolin-2-yl)cyclohexane-1-carboxamide

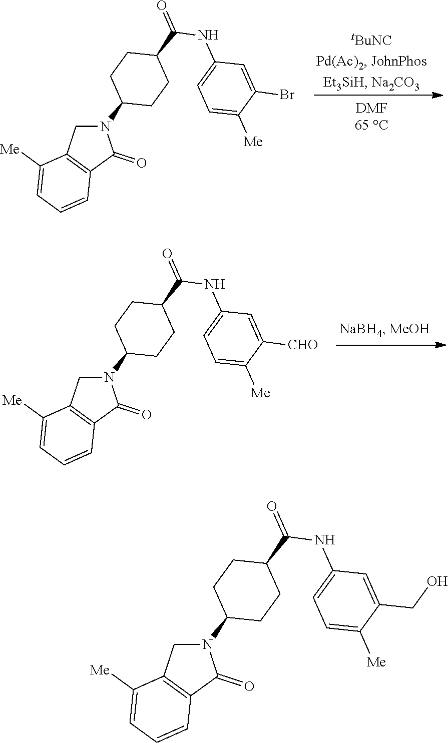 [SMILES: Cc1ccc(NC(=O)[C@H]2CC[C@@H](N3Cc4c(C)cccc4C3=O)CC2)cc1Br ; Cc1ccc(NC(=O)[C@H]2CC[C@@H](N3Cc4c(C)cccc4C3=O)CC2)cc1C=O ; Cc1ccc(NC(=O)[C@H]2CC[C@@H](N3Cc4c(C)cccc4C3=O)CC2)cc1CO]

### Step 1

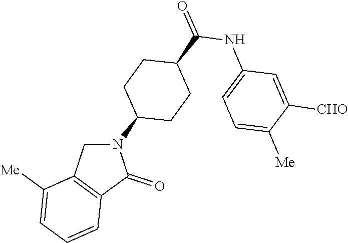 [SMILES: Cc1ccc(NC(=O)[C@H]2CC[C@@H](N3Cc4c(C)cccc4C3=O)CC2)cc1C=O]

### (1s,4s)-N-(3-Formyl-4-methylphenyl)-4-(4-methyl-1-oxoisoindolin-2-yl)cyclohexane-1-carboxamide

To a solution of (1s,4s)-N-(3-bromo-4-methylphenyl)-4-(4-methyl-1-oxoisoindolin-2-yl)cyclohexane-1-carboxamide (150 mg, 340 μmol) in DMF (7.50 mL) was added tert-butyl isocyanide (47.1 μL, 408 μmol), sodium carbonate (43.7 mg, 408 μmol), palladium (II) acetate (3.81 mg, 17.0 μmol), 2-(di-t-butylphosphino) biphenyl (10.2 mg, 34.0 μmol) and triethylsilane (181 μL, 1.12 mmol). The reaction mixture was stirred at 65° C. for 10 hours. The reaction mixture was concentrated in vacuo and the crude residue was purified by reverse phase chromatography (C18 25 g, 0 to 100% ACN in ammonium formate) to obtain (1s,4s)-N-(3-formyl-4-methylphenyl)-4-(4-methyl-1-oxoisoindolin-2-yl)cyclohexane-1-carboxamide (20.0 mg, 15% Yield); LCMS ESI m/z: 391.3 [M+H]⁺.

### Step 2

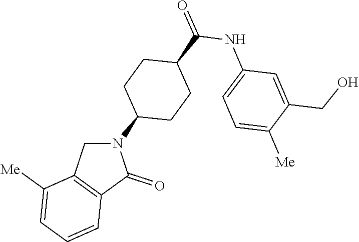 [SMILES: Cc1ccc(NC(=O)[C@H]2CC[C@@H](N3Cc4c(C)cccc4C3=O)CC2)cc1CO]

### (1s,4s)-N-(3-(Hydroxymethyl)-4-methylphenyl)-4-(4-methyl-1-oxoisoindolin-2-yl)cyclohexane-1-carboxamide

To a solution of (1s,4s)-N-(3-formyl-4-methylphenyl)-4-(4-methyl-1-oxoisoindolin-2-yl)cyclohexane-1-carboxamide (20.0 mg, 51.2 μmol) in MeOH (2.00 mL) was added sodium borohydride (2.42 mg, 61.5 μmol). The reaction mixture was stirred at room temperature for 4 h. The reaction mixture was concentrated in vacuo and the crude residue was purified by reverse phase chromatography (C18 25 g, 0 to 100% ACN in ammonium formate) to obtain the (1s,4s)-N-(3-(hydroxymethyl)-4-methylphenyl)-4-(4-methyl-1-oxoisoindolin-2-yl)cyclohexane-1-carboxamide as a white solid (13.3 mg, 66% Yield); LCMS ESI m/z: 393.4 [M+H]⁺; ¹H NMR (400 MHz, DMSO-d₆) δ 9.73 (s, 1H), 7.63 (d, J=1.6 Hz, 1H), 7.54-7.43 (m, 2H), 7.42-7.27 (m, 2H), 7.03 (d, J=8.2 Hz, 1H), 5.13 (s, 1H), 4.45 (s, 2H), 4.38 (s, 2H), 4.22-4.01 (m, 1H), 2.68 (s, 1H), 2.34 (s, 3H), 2.16 (s, 3H), 2.12 (d, J=13.0 Hz, 2H), 2.08-1.94 (m, 2H), 1.72 (dd, J=17.2, 8.7 Hz, 2H), 1.60 (dd, J=8.5, 3.6 Hz, 2H).

### Example 127

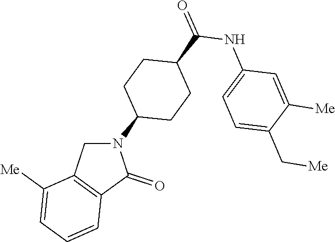 [SMILES: CCc1ccc(NC(=O)[C@H]2CC[C@@H](N3Cc4c(C)cccc4C3=O)CC2)cc1C]

### (1s,4s)-N-(4-Ethyl-3-methylphenyl)-4-(4-methyl-1-oxoisoindolin-2-yl)cyclohexane-1-carboxamide

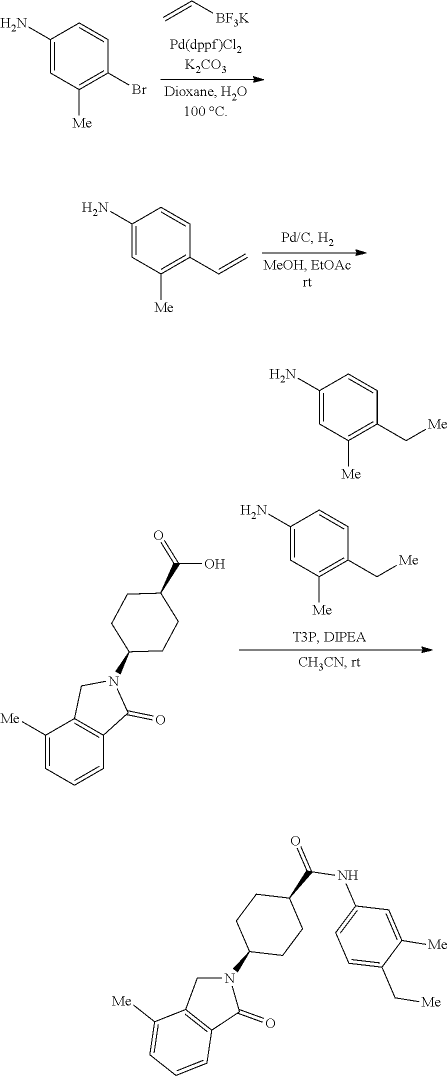

### Step 1

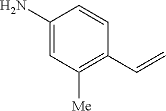

### 3-Methyl-4-vinylaniline

To a solution of 4-bromo-3-methylaniline (2.00 g, 10.7 mmol) in dioxane (50 mL) and water (5 mL), was added 1,1′-bis(diphenylphosphino)ferrocene dichloropalladium (II) (803 mg, 1.07 mmol), cesium carbonate (10.7 g, 32.2 mmol), and potassium vinyltrifluoroborate (1.63 g, 11.8 mmol). The tube was charged with N₂ and sealed. The reaction mixture was heated at 100° C. After 4 hours, the reaction mixture was concentrated in vacuo and the crude residue was purified by reverse phase chromatography (C18 25 g, 0 to 100% ACN in Ammonium formate) to obtain 3-methyl-4-vinylaniline (286 mg, 20% Yield); LCMS ESI m/z: 134.2 [M+H]⁺.

### Step 2

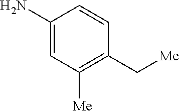

### 4-Ethyl-3-methylaniline

To a solution of 3-methyl-4-vinylaniline (286 mg, 2.15 mmol) in MeOH (2.00 mL) and EtOAc (2.00 mL) was added Palladium/C (229 mg, 215 μmol). The tube was charged with H₂ and sealed. The reaction mixture was stirred overnight at rt. The reaction mixture was filtered through Celite, concentrated in vacuo and the crude residue was purified by reverse phase chromatography (C18 25 g, 0 to 100% ACN in ammonium formate) to obtain 4-ethyl-3-methylaniline (209 mg, 72% Yield); LCMS ESI m/z: 136.2 [M+H]⁺.

### Step 3

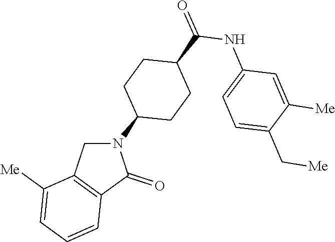 [SMILES: CCc1ccc(NC(=O)[C@H]2CC[C@@H](N3Cc4c(C)cccc4C3=O)CC2)cc1C]

### (1s,4s)-N-(4-Ethyl-3-methylphenyl)-4-(4-methyl-1-oxoisoindolin-2-yl)cyclohexane-1-carboxamide

To a solution of (1s,4s)-4-(4-methyl-1-oxoisoindolin-2-yl)cyclohexane-1-carboxylic acid (100 mg, 366 μmol) in ACN (6.00 mL) was added N,N-diisopropylethylamine (141 μL, 805 μmol), 3-methyl-4-ethylaniline (55.4 mg, 410 μmol) and 1-propylphosphonic acid cyclic anhydride 50% w in EtOAc (240 μL, 402 μmol). The reaction mixture was stirred for 24 hours at rt. The reaction mixture was concentrated in vacuo and the crude residue was purified by reverse phase chromatography (C18 25 g, 0 to 100% ACN in Ammonium formate) to obtain (1s,4s)-N-(4-ethyl-3-methylphenyl)-4-(4-methyl-1-oxoisoindolin-2-yl)cyclohexane-1-carboxamide as a white solid (69.0 mg, 48% Yield); LCMS ESI m/z: 136.2 [M+H]⁺. 1H NMR (400 MHz, DMSO-d₆) δ 9.65 (s, 1H), 7.53-7.46 (m, 1H), 7.44 (d, J=1.4 Hz, 1H), 7.41-7.29 (m, 3H), 7.04 (d, J=8.2 Hz, 1H), 4.37 (s, 2H), 4.11 (ddd, J=11.3, 7.8, 3.8 Hz, 1H), 3.34 (s, 2H), 2.67 (s, 1H), 2.34 (s, 3H), 2.23 (s, 3H), 2.12 (d, J=14.1 Hz, 2H), 2.06-1.91 (m, 2H), 1.72 (dd, J=17.3, 8.7 Hz, 2H), 1.60 (dd, J=8.6, 3.5 Hz, 2H), 1.11 (t, J=7.5 Hz, 3H).

### Example 128

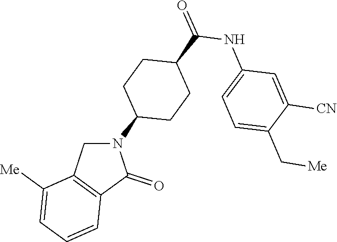 [SMILES: CCc1ccc(NC(=O)[C@H]2CC[C@@H](N3Cc4c(C)cccc4C3=O)CC2)cc1C#N]

### (1s,4s)-N-(3-Cyano-4-ethylphenyl)-4-(4-methyl-1-oxoisoindolin-2-yl)cyclohexane-1-carboxamide

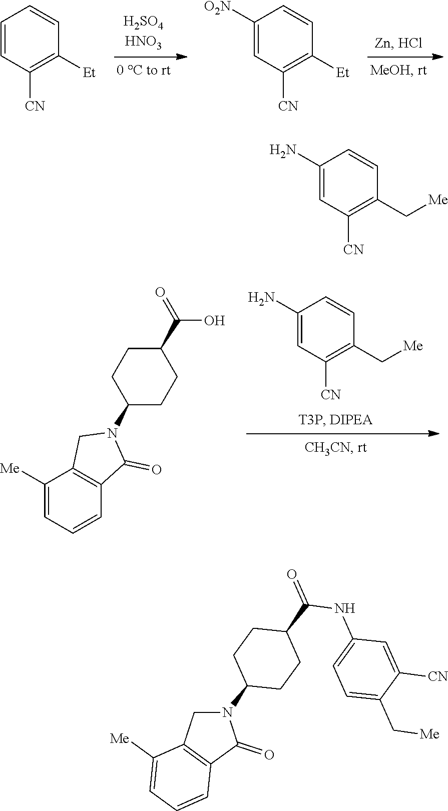

### Step 1

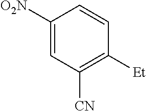 [SMILES: CCc1ccc([N+](=O)[O-])cc1C#N]

### 2-Ethyl-5-nitrobenzonitrile

To a solution of H₂SO₄ (2.4 mL) and HNO₃ (1.2 mL) at 0° C. was added 2-ethylbenzonitrile (1.20 g, 9.15 mmol). The reaction mixture was warmed to room temperature and stirred for 1 h at rt. The reaction mixture was poured onto crushed ice/water and extracted with DCM. The combined organic layers were washed with sodium bicarbonate solution and dried over sodium sulfate. The organic layer was evaporated in vacuo and the residue was purified by regular phase chromatography (Silica gel 25 g, 0 to 20% EtOAc in Heptanes) to obtain 2-ethyl-5-nitrobenzonitrile (1.31 g, 81% Yield); ¹H NMR (400 MHz, CDCl₃) δ 8.43 (s, 1H), 8.34 (d, J=8.4 Hz, 1H), 7.57 (d, J=8.5 Hz, 1H), 2.97 (dd, J=10.3, 4.5 Hz, 2H), 1.33 (dd, J=10.3, 4.8 Hz, 3H).

### Step 2

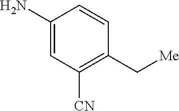 [SMILES: CCc1ccc(N)cc1C#N]

### 5-Amino-2-ethylbenzonitrile

Zinc (1.95 g, 29.7 mmol) was added to a solution of 2-ethyl-5-nitrobenzonitrile (1.31 g, 7.44 mmol) in MeOH (40.0 mL) and HCl (5.97 mL). The reaction mixture was stirred at room temperature for 2 h. Subsequently, 1M NaOH was added to basify the reaction mixture and it was then extracted with EtOAc. The combined organic phases were dried with Na₂SO₄ and concentrated in vacuo. The crude product was purified by silica gel chromatography (heptane/ethyl acetate, 70:30) to give 5-amino-2-ethylbenzonitrile (848 mg, 78% Yield); ¹H NMR (400 MHz, CD₃OD) δ 7.03 (d, J=8.1 Hz, 1H), 6.89-6.82 (m, 2H), 4.80 (s, 2H), 2.63 (q, J=7.6 Hz, 2H), 1.15 (t, J=7.6 Hz, 3H).

### Step 3

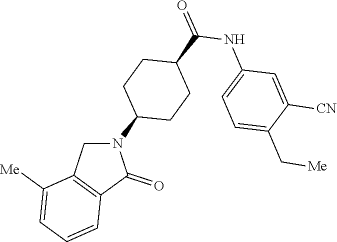 [SMILES: CCc1ccc(NC(=O)[C@H]2CC[C@@H](N3Cc4c(C)cccc4C3=O)CC2)cc1C#N]

### (1s,4s)-N-(3-Cyano-4-ethylphenyl)-4-(4-methyl-1-oxoisoindolin-2-yl)cyclohexane-1-carboxamide

To a solution of (1s,4s)-4-(4-methyl-1-oxoisoindolin-2-yl)cyclohexane-1-carboxylic acid (100 mg, 366 μmol) in ACN (6.00 mL) was added N,N-diisopropylethylamine (141 μL, 805 μmol), 5-amino-2-ethylbenzonitrile (59.9 mg, 410 μmol) and 1-propylphosphonic acid cyclic anhydride 50% w in EtOAc (240 μL, 402 μmol). The reaction mixture was stirred for 24 hours at room temperature. The reaction mixture was concentrated in vacuo and the crude residue was purified by reverse phase chromatography (25 g, 0 to 100% ACN in ammonium formate) to obtain (1s,4s)-N-(3-cyano-4-ethylphenyl)-4-(4-methyl-1-oxoisoindolin-2-yl)cyclohexane-1-carboxamide as a white solid (39.6 mg, 27% Yield). LCMS ESI m/z: 402.3 [M+H]⁺; ¹H NMR (400 MHz, DMSO-d₆) δ 10.08 (s, 1H), 8.12 (d, J=2.1 Hz, 1H), 7.75 (dd, J=8.5, 2.1 Hz, 1H), 7.55-7.45 (m, 1H), 7.42 (d, J=8.5 Hz, 1H), 7.40-7.30 (m, 2H), 4.38 (s, 2H), 4.21-3.91 (m, 1H), 2.75 (dd, J=15.2, 7.6 Hz, 3H), 2.34 (s, 3H), 2.14 (d, J=12.8 Hz, 2H), 2.05-1.89 (m, 2H), 1.75 (dd, J=17.5, 8.7 Hz, 2H), 1.67-1.52 (m, 2H), 1.20 (t, J=7.6 Hz, 3H).

### Example 129

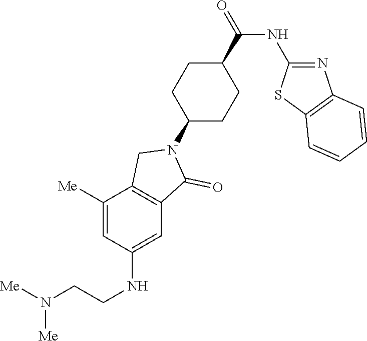 [SMILES: Cc1cc(NCCN(C)C)cc2c1CN([C@H]1CC[C@@H](C(=O)Nc3nc4ccccc4s3)CC1)C2=O]

### (1s,4s)-N-(Benzo[d]thiazol-2-yl)-4-(6-((2-(dimethylamino)ethyl)amino)-4-methyl-1-oxoisoindolin-2-yl)cyclohexane-1-carboxamide

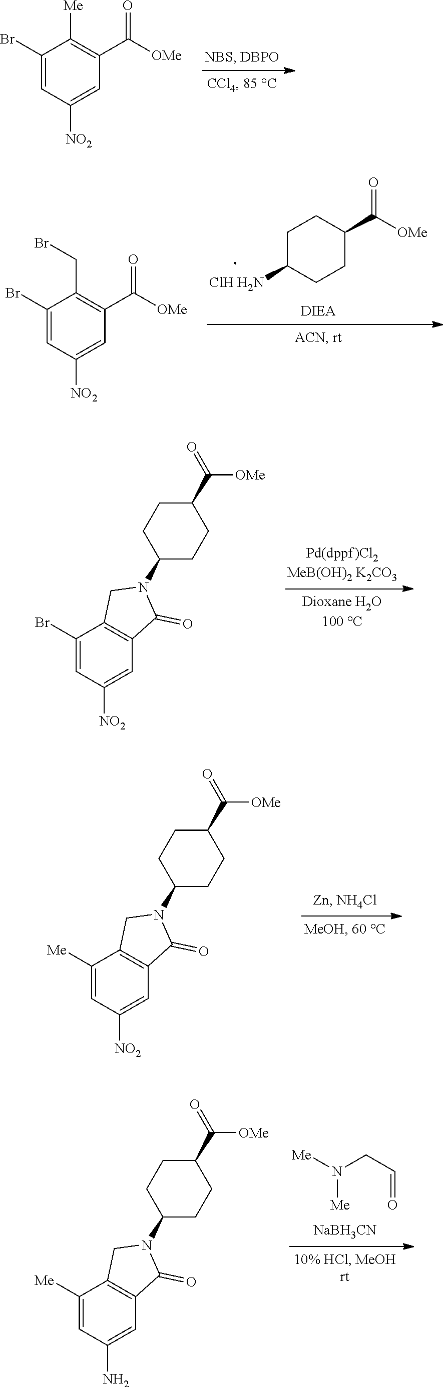

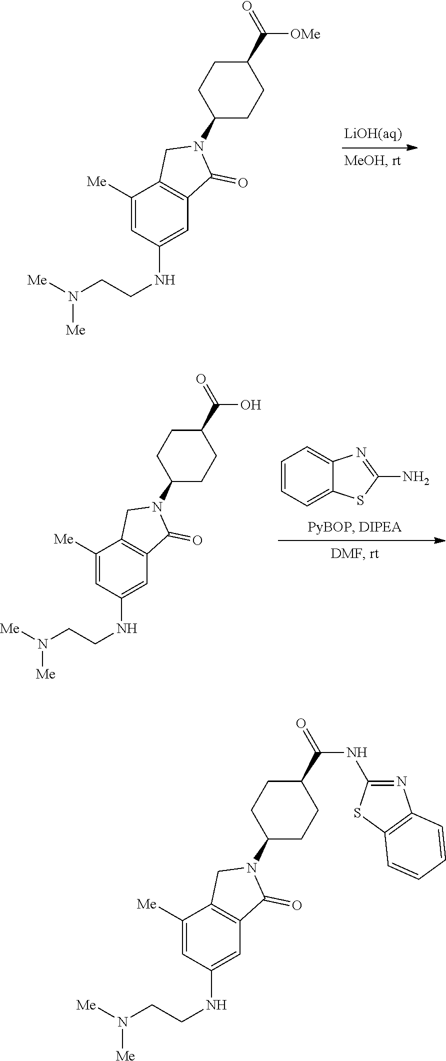

### Step 1

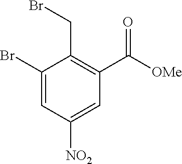 [SMILES: COC(=O)c1cc([N+](=O)[O-])cc(Br)c1CBr]

### Methyl 3-bromo-2-(bromomethyl)-5-nitrobenzoate

To a solution of methyl 3-bromo-2-methyl-5-nitrobenzoate (5.00 g, 18.2 mmol) in CCl₄ (90.0 mL) was added benzoyl peroxide (225 mg, 912 μmol) and N-bromosuccinimide (3.28 g, 18.2 mmol). The reaction mixture was refluxed at 85° C. for 16 h. The reaction mixture was cooled down and evaporated in vacuo. The residue was purified by regular phase chromatography (silica gel 25 g, 0 to 10% EtOAc in Heptanes) to obtain methyl 3-bromo-2-(bromomethyl)-5-nitrobenzoate (3.60 g, 56% Yield). ¹H NMR (400 MHz, CDCl₃) δ 8.73 (d, J=2.4 Hz, 1H), 8.60 (d, J=2.4 Hz, 1H), 5.15 (s, 2H), 4.02 (s, 3H).

### Step 2

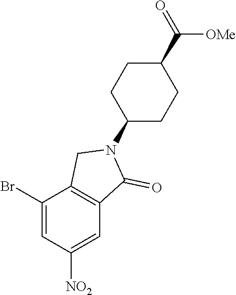 [SMILES: COC(=O)[C@H]1CC[C@@H](N2Cc3c(Br)cc([N+](=O)[O-])cc3C2=O)CC1]

### Methyl (1s,4s)-4-(4-bromo-6-nitro-1-oxoisoindolin-2-yl)cyclohexane-1-carboxylate

To a solution of methyl 3-bromo-2-methyl-5-nitrobenzoate (3.00 g, 8.50 mmol) in ACN (90.0 mL) was added cis-4-amino-cyclohexylcarboxylic acid methyl ester hydrochloride (1.53 g, 9.35 mmol) and N,N-diisopropylethylamine (3.27 mL, 18.7 mmol). The reaction mixture was stirred at room temperature for 4 h. The reaction mixture was evaporated in vacuo and the residue was purified by regular phase chromatography (silica gel 100 g, 0 to 50% EtOAc in Heptanes) to obtain methyl (1s,4s)-4-(4-bromo-6-nitro-1-oxoisoindolin-2-yl)cyclohexane-1-carboxylate (1.79 g, 53% Yield); LCMS ESI m/z: 399.65 [M+2H]⁺.

### Step 3

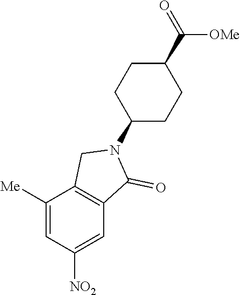 [SMILES: COC(=O)[C@H]1CC[C@@H](N2Cc3c(C)cc([N+](=O)[O-])cc3C2=O)CC1]

### Methyl (1s,4s)-4-(4-methyl-6-nitro-1-oxoisoindolin-2-yl)cyclohexane-1-carboxylate

To a solution of methyl (1s,4s)-4-(4-bromo-6-nitro-1-oxoisoindolin-2-yl)cyclohexane-1-carboxylate (3.00 g, 7.55 mmol) in dioxane (100 mL) and water (20.0 mL) was added potassium carbonate (3.20 g, 22.7 mmol), methylboronic acid (543 mg, 9.06 mmol) and 1,1′-bis(diphenylphosphino)ferrocene dichloropalladium (II) (582 mg, 755 μmol). The reaction mixture was heated at 100° C. for 8 h. The reaction mixture was cooled down and evaporated in vacuo. The residue was purified by regular phase chromatography (silica gel 100 g, 0 to 50% EtOAc in Heptanes) to obtain the product methyl (1s,4s)-4-(4-methyl-6-nitro-1-oxoisoindolin-2-yl)cyclohexane-1-carboxylate (1.43 g, 57% Yield); LCMS ESI m/z: 333.1 [M+H]⁺.

### Step 4

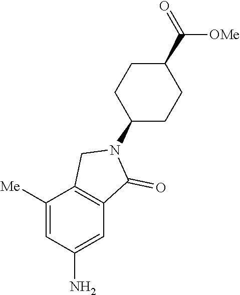 [SMILES: COC(=O)[C@H]1CC[C@@H](N2Cc3c(C)cc(N)cc3C2=O)CC1]

### Methyl (1s,4s)-4-(6-amino-4-methyl-1-oxoisoindolin-2-yl)cyclohexane-1-carboxylate

To a solution of methyl (1s,4s)-4-(4-methyl-6-nitro-1-oxoisoindolin-2-yl)cyclohexane-1-carboxylate (210 mg, 632 μmol) in MeOH (5 mL) was added zinc (169 mg, 2.53 mmol) and ammonium chloride (135 mg, 2.53 mmol). The reaction mixture was heated at 85° C. for 2 h. The reaction mixture was cooled, filtered through celite and evaporated in vacuo to obtain crude residue. The residue was dissolved in ethyl acetate, washed with water, dried over sodium sulfate and evaporated in vacuum to obtain crude methyl (1s,4s)-4-(6-amino-4-methyl-1-oxoisoindolin-2-yl)cyclohexane-1-carboxylate (182 mg, 95%, crude yield); LCMS ESI m/z: 303.1 [M+H]⁺.

### Step 5

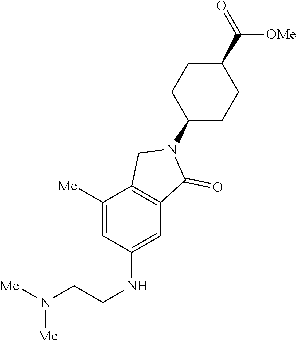 [SMILES: COC(=O)[C@H]1CC[C@@H](N2Cc3c(C)cc(NCCN(C)C)cc3C2=O)CC1]

### Methyl (1s,4s)-4-(6-((2-(dimethylamino)ethyl)amino)-4-methyl-1-oxoisoindolin-2-yl)cyclohexane-1-carboxylate

To a solution of methyl (1s,4s)-4-(6-amino-4-methyl-1-oxoisoindolin-2-yl)cyclohexane-1-carboxylate (140 mg, 463 μmol) in MeOH (5.00 mL) was added 2-(dimethylamino)acetaldehyde (121 mg, 1.39 mmol) and 2 drops of 10% HCl. After 5 min, sodium cyanoborohydride (153 mg, 2.32 mmol) was added and the reaction mixture was stirred for 3 h at rt. The reaction mixture was evaporated in vacuo and purified by regular phase chromatography (silica gel 25 g, 0 to 20% MeOH in DCM) to obtain methyl (1s,4s)-4-(6-((2-(dimethylamino)ethyl)amino)-4-methyl-1-oxoisoindolin-2-yl)cyclohexane-1-carboxylate (189 mg, 84% Yield); LCMS ESI m/z: 374.3 [M+H]⁺.

### Step 6

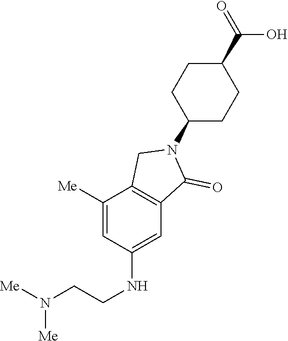 [SMILES: Cc1cc(NCCN(C)C)cc2c1CN([C@H]1CC[C@@H](C(=O)O)CC1)C2=O]

### (1s,4s)-4-(6-((2-(Dimethylamino)ethyl)amino)-4-methyl-1-oxoisoindolin-2-yl)cyclohexane-1-carboxylic acid

To a solution of methyl (1s,4s)-4-(6-((2-(dimethylamino)ethyl)amino)-4-methyl-1-oxoisoindolin-2-yl)cyclohexane-1-carboxylate (189 mg, 506 μmol) in MeOH (2 mL) was added 1M aq. KOH (506 μL, 506 μmol). The reaction mixture was stirred for 8 h at rt. The reaction mixture was quenched with 1M HCl (bringing to pH 7) and evaporated in vacuo. The residue was purified by regular phase chromatography (silica gel 25 g, 0 to 20% MeOH in DCM) to obtain (1s,4s)-4-(6-((2-(dimethylamino)ethyl)amino)-4-methyl-1-oxoisoindolin-2-yl)cyclohexane-1-carboxylic acid (113 mg, 62% Yield); LCMS ESI m/z: 360.3 [M+H]⁺.

### Step 7

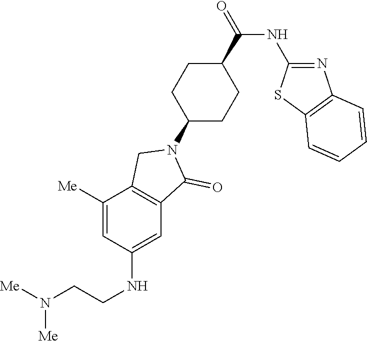 [SMILES: Cc1cc(NCCN(C)C)cc2c1CN([C@H]1CC[C@@H](C(=O)Nc3nc4ccccc4s3)CC1)C2=O]

### (1s,4s)-N-(benzo[d]thiazol-2-yl)-4-(6-((2-(dimethylamino)ethyl)amino)-4-methyl-1-oxoisoindolin-2-yl)cyclohexane-1-carboxamide

To a solution of (1s,4s)-4-(6-((2-(dimethylamino)ethyl)amino)-4-methyl-1-oxoisoindolin-2-yl)cyclohexane-1-carboxylic acid (42.0 mg, 117 μmol) in ACN (2.00 mL) was added 2-aminobenzothiazole (19.9 mg, 129 μmol), N,N-diisopropylethylamine (45.0 μL, 257 μmol) and (benzotriazol-1-yloxy)tripyrrolidinophosphonium hexafluorophosphate (PyBOP) (68.2 mg, 129 μmol). The reaction mixture was stirred overnight at rt. The reaction mixture was concentrated in vacuo and the crude residue was purified by reverse phase chromatography (25 g, 0 to 100% ACN in ammonium formate) to obtain (1s,4s)-N-(benzo[d]thiazol-2-yl)-4-(6-((2-(dimethylamino)ethyl)amino)-4-methyl-1-oxoisoindolin-2-yl)cyclohexane-1-carboxamide as a white solid (7.66 mg, 13% Yield); LCMS ESI m/z: 492.2 [M+H]⁺; ¹H NMR (400 MHz, DMSO-d₆) δ 8.24 (s, 1H), 7.97 (d, J=7.5 Hz, 1H), 7.73 (d, J=7.9 Hz, 1H), 7.55-7.37 (m, 1H), 7.35-7.20 (m, 1H), 6.63 (d, J=7.1 Hz, 2H), 5.58 (s, 1H), 4.20 (s, 2H), 4.09 (dd, J=13.0, 9.4 Hz, 2H), 3.13 (t, J=6.4 Hz, 2H), 2.89 (s, 1H), 2.47 (s, 1H), 2.22 (s, 6H), 2.21 (s, 3H), 2.16 (d, J=12.9 Hz, 2H), 1.89 (t, J=12.0 Hz, 2H), 1.77 (dd, J=17.7, 8.4 Hz, 2H), 1.68-1.54 (m, 2H).

### Example 130

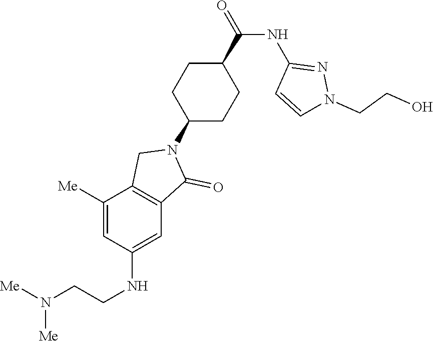 [SMILES: Cc1cc(NCCN(C)C)cc2c1CN([C@H]1CC[C@@H](C(=O)Nc3ccn(CCO)n3)CC1)C2=O]

### (1s,4s)-4-(6-((2-(Dimethylamino)ethyl)amino)-4-methyl-1-oxoisoindolin-2-yl)-N-(1-(2-hydroxyethyl)-1H-pyrazol-3-yl)cyclohexane-1-carboxamide

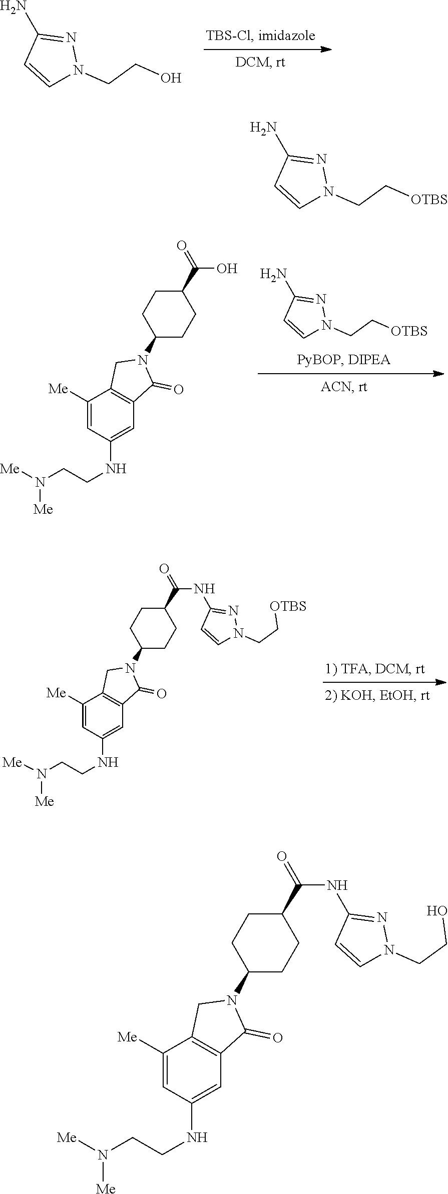 [SMILES: Nc1ccn(CCO)n1 ; CC(C)(C)[Si](C)(C)OCCn1ccc(N)n1 ; Cc1cc(NCCN(C)C)cc2c1CN([C@H]1CC[C@@H](C(=O)O)CC1)C2=O ; CC(C)(C)[Si](C)(C)OCCn1ccc(N)n1 ; Cc1cc(NCCN(C)C)cc2c1CN([C@H]1CC[C@@H](C(=O)Nc3ccn(CCO[Si](C)(C)C(C)(C)C)n3)CC1)C2=O ; Cc1cc(NCCN(C)C)cc2c1CN([C@H]1CC[C@@H](C(=O)Nc3ccn(CCO)n3)CC1)C2=O]

### Step 1

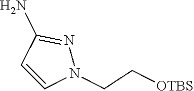 [SMILES: CC(C)(C)[Si](C)(C)OCCn1ccc(N)n1]

### 1-(2-((tert-Butyldimethylsilyl)oxy)ethyl)-1H-pyrazol-3-amine

To a solution of 2-(3-amino-1H-pyrazol-1-yl) ethanol (500 mg, 3.74 mmol) in DCM (5 mL) was added imidazole (617 mg, 8.97 mmol) and TBS-CI (683 mg, 4.48 mmol). The reaction mixture was stirred overnight at rt. The reaction mixture was concentrated in vacuo and the residue was purified by regular phase chromatography (silica gel 25 g, 0 to 20% MeOH in DCM) to obtain 1-(2-((tert-butyldimethylsilyl)oxy)ethyl)-1H-pyrazol-3-amine (857 mg, 95% Yield); LCMS ESI m/z: 242.3 [M+H]⁺.

### Step 2

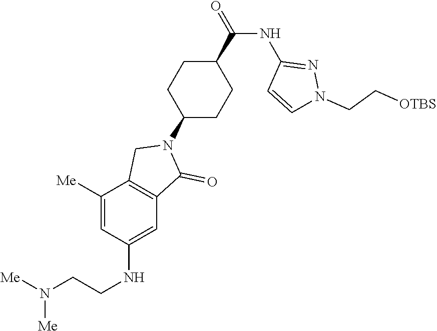 [SMILES: Cc1cc(NCCN(C)C)cc2c1CN([C@H]1CC[C@@H](C(=O)Nc3ccn(CCO[Si](C)(C)C(C)(C)C)n3)CC1)C2=O]

### (1s,4s)-N-(1-(2-((tert-Butyldimethylsilyl)oxy)ethyl)-1H-pyrazol-3-yl)-4-(6-((2-(dimethylamino)ethyl)amino)-4-methyl-1-oxoisoindolin-2-yl)cyclohexane-1-carboxamide

To a solution of (1s,4s)-4-(6-((2-(dimethylamino)ethyl)amino)-4-methyl-1-oxoisoindolin-2-yl)cyclohexane-1-carboxylic acid (50.0 mg, 139 μmol) in ACN (2 mL) was added N,N-diisopropylethylamine (53.6 μL, 306 μmol), (benzotriazol-1-yloxy)tripyrrolidinophosphonium hexafluorophosphate (PyBOP) (81.2 mg, 153 μmol) and 1-(2-((tert-butyldimethylsilyl)oxy)ethyl)-1H-pyrazol-3-amine (36.9 mg, 153 μmol). The reaction mixture was stirred overnight at rt. The reaction mixture was evaporated in vacuo and the residue was purified by regular phase chromatography (silica gel 25 g, 0 to 20% MeOH in DCM) to obtain (1s,4s)-N-(1-(2-((tert-butyldimethylsilyl)oxy)ethyl)-1H-pyrazol-3-yl)-4-(6-((2-(dimethylamino)ethyl)amino)-4-methyl-1-oxoisoindolin-2-yl)cyclohexane-1-carboxamide (41.3 mg, 51% Yield); LCMS ESI m/z: 585.3 [M+H]⁺.

### Step 3

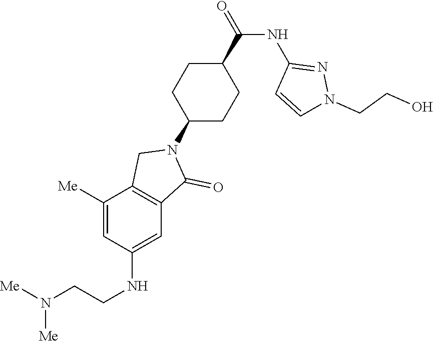 [SMILES: Cc1cc(NCCN(C)C)cc2c1CN([C@H]1CC[C@@H](C(=O)Nc3ccn(CCO)n3)CC1)C2=O]

### (1s,4s)-4-(6-((2-(Dimethylamino)ethyl)amino)-4-methyl-1-oxoisoindolin-2-yl)-N-(1-(2-hydroxyethyl)-1H-pyrazol-3-yl)cyclohexane-1-carboxamide

To a solution of (1s,4s)-N-(1-(2-((tert-butyldimethylsilyl)oxy)ethyl)-1H-pyrazol-3-yl)-4-(6-((2-(dimethylamino)ethyl)amino)-4-methyl-1-oxoisoindolin-2-yl)cyclohexane-1-carboxamide (41.3 mg, 70.9 μmol) in DCM (2.00 mL) was added TFA (2.00 mL). The reaction mixture was stirred overnight at rt. The reaction mixture was concentrated to obtain a crude residue. The residue was diluted with ethyl acetate. Then, saturated sodium bicarbonate was added to the solution and the mixture was extracted with ethyl acetate three times. The combined organic phase was dried over Na₂SO₄, filtered, and concentrated under reduced pressure to give an oil. To the crude residue was added EtOH (2.00 mL) and 1M aq. KOH (70.9 μL, 70.9 μmol). The resulting solution was stirred for 1.5 h at room temperature. The reaction mixture was concentrated in vacuo and the crude residue was purified by reverse phase chromatography (C18 25 g, 0 to 100% ACN in Ammonium formate-30 CV gradient) to obtain (1s,4s)-4-(6-((2-(dimethylamino)ethyl)amino)-4-methyl-1-oxoisoindolin-2-yl)-N-(1-(2-hydroxyethyl)-1H-pyrazol-3-yl)cyclohexane-1-carboxamide (23.0 mg, 69% Yield); LCMS ESI m/z: 469.2 [M+H]⁺; ¹H NMR (400 MHz, DMSO-d₆) δ 10.30 (s, 1H), 8.30 (s, 1H), 7.55 (d, J=1.9 Hz, 1H), 6.62 (d, J=9.3 Hz, 2H), 6.48 (d, J=2.0 Hz, 1H), 5.60 (s, 2H), 4.18 (s, 2H), 4.07 (d, J=11.3 Hz, 1H), 4.01 (t, J=5.5 Hz, 2H), 3.69 (t, J=5.6 Hz, 2H), 3.13 (s, 2H), 2.66 (s, 1H), 2.54 (s, 1H), 2.23 (s, 6H), 2.20 (s, 3H), 2.07 (d, J=12.1 Hz, 2H), 2.00-1.85 (m, 2H), 1.66 (dd, J=17.2, 8.6 Hz, 2H), 1.54 (d, J=9.3 Hz, 2H).

### Example 131

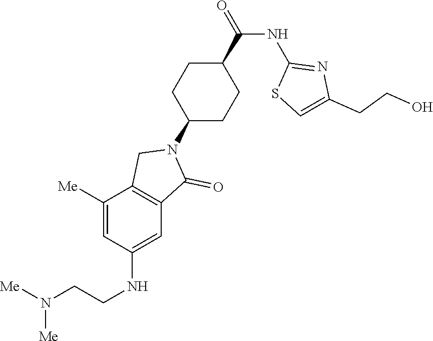 [SMILES: Cc1cc(NCCN(C)C)cc2c1CN([C@H]1CC[C@@H](C(=O)Nc3nc(CCO)cs3)CC1)C2=O]

### (1s,4s)-4-(6-((2-(Dimethylamino)ethyl)amino)-4-methyl-1-oxoisoindolin-2-yl)-N-(4-(2-hydroxyethyl) thiazol-2-yl)cyclohexane-1-carboxamide

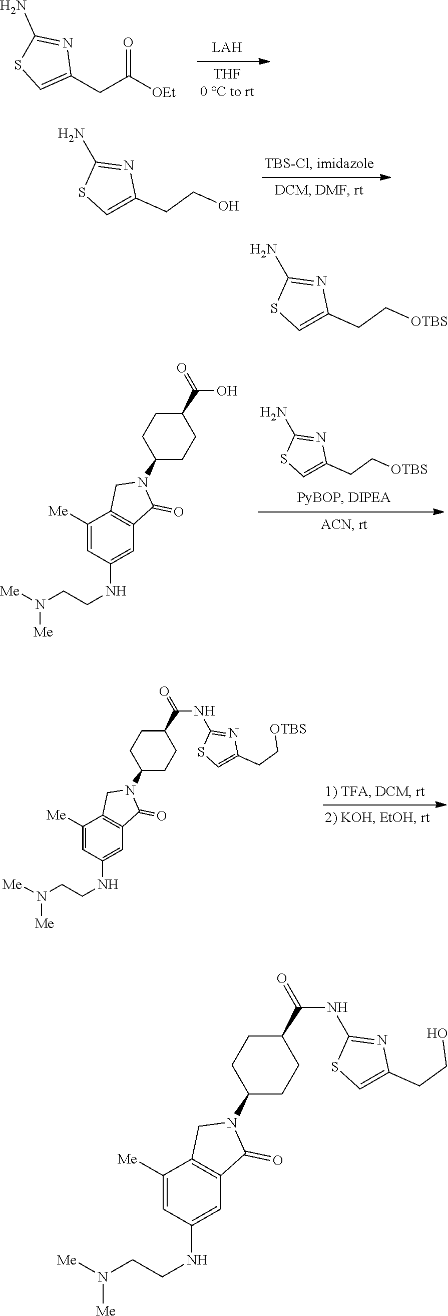

### Step 1

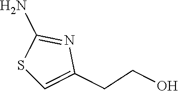

### 2-(2-Aminothiazol-4-yl) ethan-1-ol

To a solution of ethyl 2-aminothiazole-4-acetate (500 mg, 2.63 mmol) in THF (5.00 mL) was added lithium aluminum hydride (126 mg, 3.16 mmol). The reaction mixture was stirred at room temperature for 4 h. The reaction mixture was quenched by water. The crude residue was extracted with EtOAc and evaporated to obtain crude 2-(2-aminothiazol-4-yl) ethan-1-ol (133 mg, 35%, crude yield); LCMS ESI m/z: 144.9 [M+H]⁺.

### Step 2

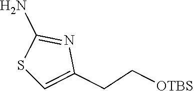 [SMILES: CC(C)(C)[Si](C)(C)OCCc1csc(N)n1]

### 4-(2-((tert-Butyldimethylsilyl)oxy)ethyl)thiazol-2-amine

To a solution of 2-(2-aminothiazol-4-yl) ethan-1-ol (133 mg, 922 μmol) in DCM (1.00 mL) and DMF (1.00 mL) was added imidazole (140 mg, 2.03 mmol) and TBS-CI (154 mg, 1.01 mmol). The reaction mixture was stirred overnight at rt. The reaction mixture was concentrated in vacuo and the residue was purified by regular phase chromatography (silica gel 25 g, 0 to 20% MeOH in DCM) to obtain 4-(2-((tert-butyldimethylsilyl)oxy)ethyl)thiazol-2-amine (160 mg, 67% Yield); LCMS ESI m/z: 259.0 [M+H]⁺.

### Step 3

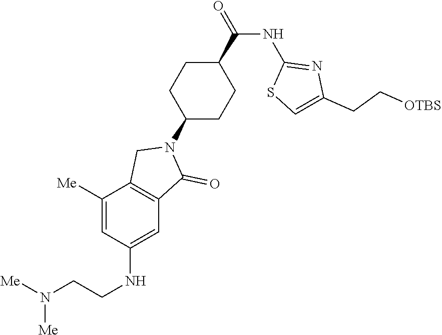 [SMILES: Cc1cc(NCCN(C)C)cc2c1CN([C@H]1CC[C@@H](C(=O)Nc3nc(CCO[Si](C)(C)C(C)(C)C)cs3)CC1)C2=O]

### (1s,4s)-N-(4-(2-((tert-Butyldimethylsilyl)oxy)ethyl)thiazol-2-yl)-4-(6-((2-(dimethylamino)ethyl)amino)-4-methyl-1-oxoisoindolin-2-yl)cyclohexane-1-carboxamide

To a solution of (1s,4s)-4-(6-((2-(dimethylamino)ethyl)amino)-4-methyl-1-oxoisoindolin-2-yl)cyclohexane-1-carboxylic acid (100 mg, 278 μmol) in ACN (4.00 mL) was added N, N-diisopropylethylamine (107 μL, 612 μmol), (benzotriazol-1-yloxy)tripyrrolidinophosphonium hexafluorophosphate (PyBOP) (162 mg, 306 μmol) and 4-(2-((tert-butyldimethylsilyl)oxy)ethyl)thiazol-2-amine (79.1 mg, 306 μmol). The reaction mixture was stirred overnight at rt. The reaction mixture was evaporated in vacuo and the residue was purified by regular phase chromatography (silica gel 25 g, 0 to 20% MeOH in DCM) to obtain (1s,4s)-N-(4-(2-((tert-butyldimethylsilyl)oxy)ethyl)thiazol-2-yl)-4-(6-((2-(dimethylamino)ethyl)amino)-4-methyl-1-oxoisoindolin-2-yl)cyclohexane-1-carboxamide (70.1 mg, 42% Yield); LCMS ESI m/z: 600.3 [M+H]⁺.

### Step 4

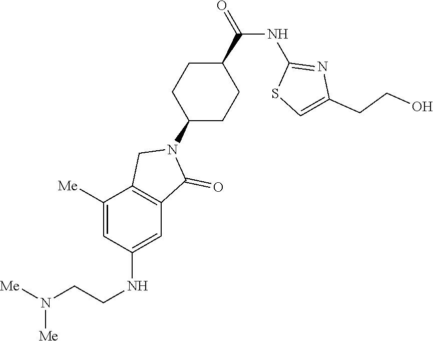 [SMILES: Cc1cc(NCCN(C)C)cc2c1CN([C@H]1CC[C@@H](C(=O)Nc3nc(CCO)cs3)CC1)C2=O]

### (1s,4s)-4-(6-((2-(Dimethylamino)ethyl)amino)-4-methyl-1-oxoisoindolin-2-yl)-N-(4-(2-hydroxyethyl)thiazol-2-yl)cyclohexane-1-carboxamide

The solution of (1s,4s)-N-(4-(2-((tert-butyldimethylsilyl)oxy)ethyl)thiazol-2-yl)-4-(6-((2-(dimethylamino)ethyl)amino)-4-methyl-1-oxoisoindolin-2-yl)cyclohexane-1-carboxamide (70.1 mg, 117 μmol) in DCM (5.0 mL) and TFA (5.0 mL) was stirred overnight at rt. The reaction mixture was concentrated to obtain a crude residue. The residue was diluted with ethyl acetate. Then saturated sodium bicarbonate (aq.) was added to the solution and the mixture was extracted with ethyl acetate three times. The combined organic phase was dried over Na₂SO₄, filtered, and concentrated under reduced pressure to give an oil. To the crude residue was added EtOH (2.00 mL) and 1M aq. KOH (117 μL, 117 μmol). The resulting solution was stirred for 1.5 h at rt. The reaction mixture was concentrated in vacuo and the crude residue was purified by reverse phase chromatography (C18 25 g, 0 to 100% ACN in ammonium formate) to obtain (1s,4s)-4-(6-((2-(dimethylamino)ethyl)amino)-4-methyl-1-oxoisoindolin-2-yl)-N-(4-(2-hydroxyethyl) thiazol-2-yl)cyclohexane-1-carboxamide (35.8 mg, 63% Yield); LCMS ESI m/z: 600.3 [M+H]⁺; ¹H NMR (400 MHz, DMSO-d₆) δ 12.02 (s, 1H), 6.79 (s, 1H), 6.74-6.46 (m, 2H), 5.53 (t, J=5.4 Hz, 1H), 4.62 (t, J=5.3 Hz, 1H), 4.18 (s, 2H), 4.06 (t, J=11.2 Hz, 1H), 3.67 (dd, J=12.4, 7.0 Hz, 2H), 3.10 (dd, J=12.2, 6.3 Hz, 2H), 2.79 (s, 1H), 2.74 (t, J=7.0 Hz, 2H), 2.42 (t, J=6.6 Hz, 2H), 2.20 (s, 3H), 2.17 (s, 6H), 2.10 (d, J=11.3 Hz, 2H), 1.88 (dd, J=22.8, 10.7 Hz, 2H), 1.72 (dd, J=17.6, 8.6 Hz, 2H), 1.58 (d, J=9.2 Hz, 2H).

### Example 132

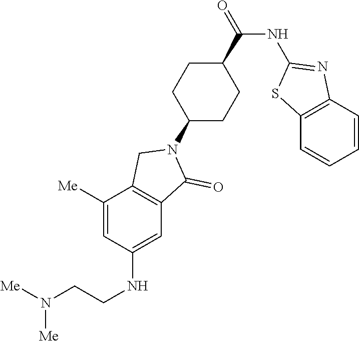 [SMILES: Cc1cc(NCCN(C)C)cc2c1CN([C@H]1CC[C@@H](C(=O)Nc3nc4ccccc4s3)CC1)C2=O]

### (1s,4s)-4-(6-((2-(Dimethylamino)ethyl)amino)-4-methyl-1-oxoisoindolin-2-yl)-N-(1H-indol-2-yl)cyclohexane-1-carboxamide

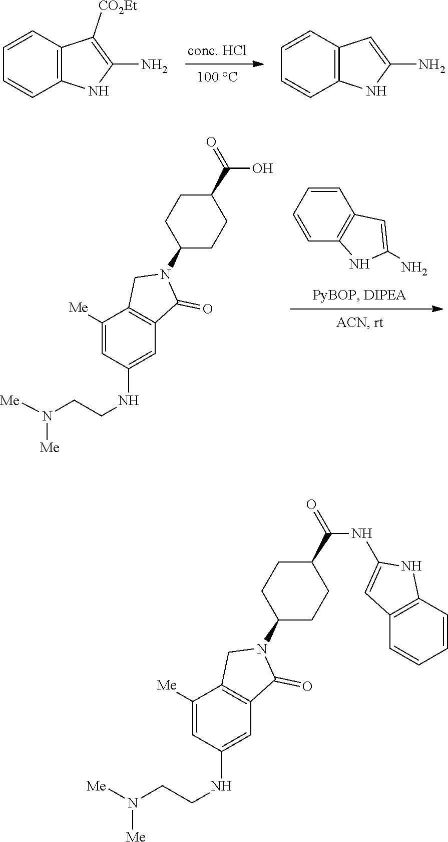 [SMILES: CCOC(=O)c1c(N)[nH]c2ccccc12 ; Nc1cc2ccccc2[nH]1 ; Cc1cc(NCCN(C)C)cc2c1CN([C@H]1CC[C@@H](C(=O)O)CC1)C2=O ; Nc1cc2ccccc2[nH]1 ; Cc1cc(NCCN(C)C)cc2c1CN([C@H]1CC[C@@H](C(=O)Nc3cc4ccccc4[nH]3)CC1)C2=O]

### Step 1

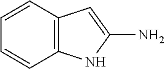

### 1H-Indol-2-amine

The solution of ethyl 2-aminoindole-3-carboxylate (0.600 g, 2.94 mmol) in HCl (2 mL) was heated at 100° C. for 2 h. The reaction mixture was evaporated in vacuo and the crude residue was dissolved in MeOH (10 mL) and evaporated again to obtain crude 1H-indol-2-amine (233 mg, 60% Yield); LCMS ESI m/z: 133.0 [M+H]⁺.

### Step 2

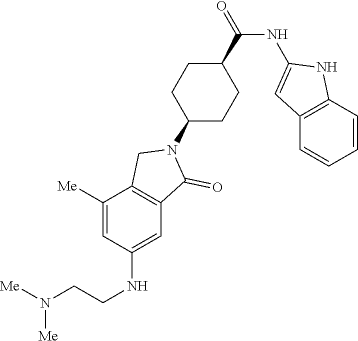 [SMILES: Cc1cc(NCCN(C)C)cc2c1CN([C@H]1CC[C@@H](C(=O)Nc3cc4ccccc4[nH]3)CC1)C2=O]

### (1s,4s)-4-(6-((2-(Dimethylamino)ethyl)amino)-4-methyl-1-oxoisoindolin-2-yl)-N-(1H-indol-2-yl)cyclohexane-1-carboxamide

To a solution of (1s,4s)-4-(6-((2-(dimethylamino)ethyl)amino)-4-methyl-1-oxoisoindolin-2-yl)cyclohexane-1-carboxylic acid (100 mg, 278 μmol) in ACN (4.00 mL) was added N,N-diisopropylethylamine (107 μL, 612 μmol), (benzotriazol-1-yloxy)tripyrrolidinophosphonium hexafluorophosphate (PyBOP) (162 mg, 306 μmol) and 1H-indol-2-amine (40.4 mg, 306 μmol). The reaction mixture was stirred overnight at rt. The reaction mixture was concentrated in vacuo and the crude residue was purified by reverse phase chromatography (C18 25 g, 0 to 100% ACN in ammonium formate) to obtain (1s,4s)-4-(6-((2-(dimethylamino)ethyl)amino)-4-methyl-1-oxoisoindolin-2-yl)-N-(1H-indol-2-yl)cyclohexane-1-carboxamide (3.70 mg, 2.8% Yield); LCMS ESI m/z: 474.5 [M+H]⁺; ¹H NMR (400 MHz, DMSO-d₆) δ 11.25 (s, 1H), 10.76 (s, 1H), 7.67-7.10 (m, 3H), 6.95-6.86 (m, 2H), 6.57 (s, 2H), 6.02 (s, 1H), 4.19 (s, 1H), 4.07 (t, J=11.3 Hz, 2H), 2.73 (s, 2H), 2.52 (s, 1H), 2.14 (d, J=11.7 Hz, 9H), 2.05 (s, 1H), 1.95 (dd, J=24.4, 12.6 Hz, 4H), 1.72 (t, J=13.1 Hz, 3H), 1.58 (d, J=9.4 Hz, 2H).

### Example 133

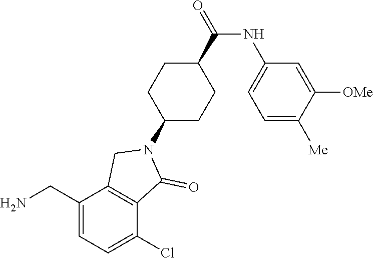 [SMILES: COc1cc(NC(=O)[C@H]2CC[C@@H](N3Cc4c(CN)ccc(Cl)c4C3=O)CC2)ccc1C]

### (1s,4s)-4-(4-(Aminomethyl)-7-chloro-1-oxoisoindolin-2-yl)-N-(3-methoxy-4-methylphenyl)cyclohexane-1-carboxamide

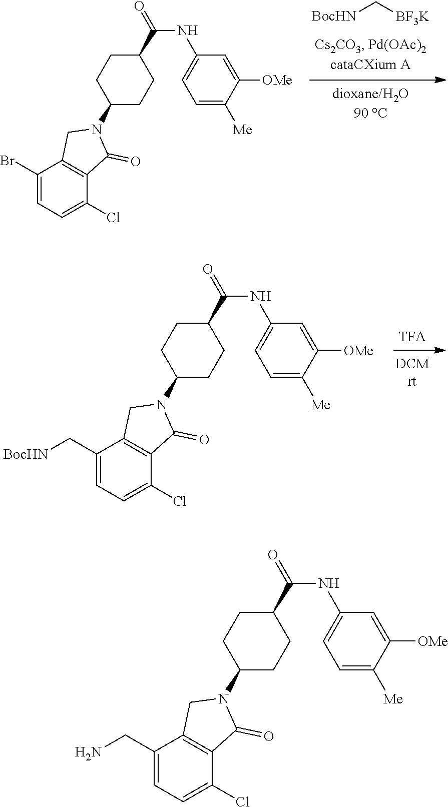

### Step 1

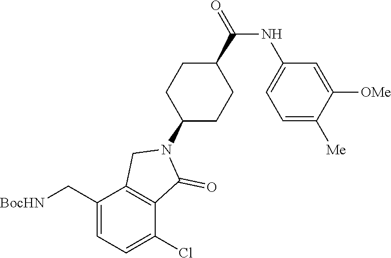 [SMILES: COc1cc(NC(=O)[C@H]2CC[C@@H](N3Cc4c(CNC(=O)OC(C)(C)C)ccc(Cl)c4C3=O)CC2)ccc1C]

### tert-Butyl ((7-chloro-2-((1s,4s)-4-((3-methoxy-4-methylphenyl)carbamoyl)cyclohexyl)-1-oxoisoindolin-4-yl)methyl)carbamate

A microwave vial was charged with potassium (((tert-butoxycarbonyl)amino)methyl)trifluoroborate (68.6 mg, 281 μmol), (1s,4s)-4-(4-bromo-7-chloro-1-oxoisoindolin-2-yl)-N-(3-methoxy-4-methylphenyl)cyclohexanecarboxamide (115 mg, 234 μmol) and cesium carbonate (229 mg, 701 μmol). Water (390 μL) and 1,4-dioxane (1.95 mL) were added, the vial was capped with a septum and the reaction mixture was degassed with a nitrogen flow for 10 minutes. Finally, palladium (II) acetate (5.25 mg, 23.4 μmol) and butyldi-1-adamantylphosphine (17.7 mg, 46.8 μmol) were then added to the vial under nitrogen and the reaction mixture was capped and stirred at 90° C. for 2 h. The reaction mixture was cooled to room temperature, silica gel was added directly and the dry loaded crude mixture was purified by silica gel flash chromatography eluting from 10 to 80% EtOAc in heptanes to yield tert-butyl ((7-chloro-2-((1s,4s)-4-((3-methoxy-4-methylphenyl)carbamoyl)cyclohexyl)-1-oxoisoindolin-4-yl)methyl)carbamate (86.6 mg, 68% Yield) as a white solid; LCMS ESI m/z: 542.0 [M+H]⁺.

### Step 2

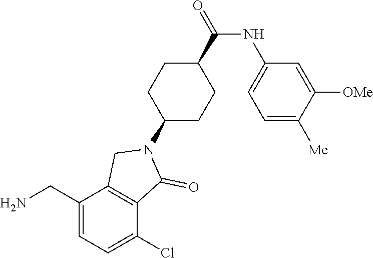 [SMILES: COc1cc(NC(=O)[C@H]2CC[C@@H](N3Cc4c(CN)ccc(Cl)c4C3=O)CC2)ccc1C]

### (1s,4s)-4-(4-(Aminomethyl)-7-chloro-1-oxoisoindolin-2-yl)-N-(3-methoxy-4-methylphenyl)cyclohexane-1-carboxamide

To a solution of tert-butyl ((7-chloro-2-((1s,4s)-4-((3-methoxy-4-methylphenyl)carbamoyl)cyclohexyl)-1-oxoisoindolin-4-yl)methyl)carbamate (16.0 mg, 29.5 μmol) in DCM (200 μL) was added trifluoroacetic acid (200 μL, 2.61 mmol) and the reaction mixture was stirred at room temperature for 1 h. The reaction mixture was concentrated to dryness and the crude residue was purified by reverse phase flash chromatography on C18 (30 g column, from 0 to 60% MeCN in water-10 mM ammonium formate pH=3.8) to yield (1s,4s)-4-(4-(Aminomethyl)-7-chloro-1-oxoisoindolin-2-yl)-N-(3-methoxy-4-methylphenyl)cyclohexane-1-carboxamide (4.10 mg, 31% Yield) as a white solid; LCMS ESI m/z: 442.3 [M+H]⁺; ¹H NMR (400 MHz, DMSO-d₆) δ 9.72 (s, 1H), 8.25 (s, 1H), 7.55 (d, J=8.1 Hz, 1H), 7.44 (d, J=8.1 Hz, 1H), 7.32 (d, J=1.5 Hz, 1H), 7.13 (dd, J=8.1, 1.7 Hz, 1H), 7.02 (d, J=8.2 Hz, 1H), 4.48 (s, 2H), 4.14-4.04 (m, 1H), 3.83 (s, 2H), 3.76 (s, 3H), 2.71-2.63 (m, 1H), 2.13 (d, J=12.5 Hz, 2H), 2.08 (s, 3H), 1.99 (qd, J=12.4, 3.1 Hz, 2H), 1.80-1.67 (m, 2H), 1.61 (dd, J=12.3, 3.3 Hz, 2H).

### Example 134

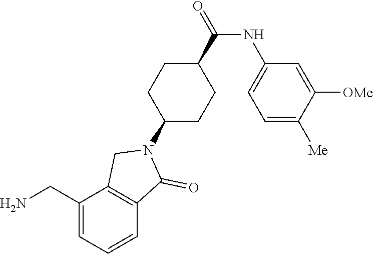 [SMILES: COc1cc(NC(=O)[C@H]2CC[C@@H](N3Cc4c(CN)cccc4C3=O)CC2)ccc1C]

### (1s,4s)-4-(4-(aminomethyl)-1-oxoisoindolin-2-yl)-N-(3-methoxy-4-methylphenyl)cyclohexane-1-carboxamide

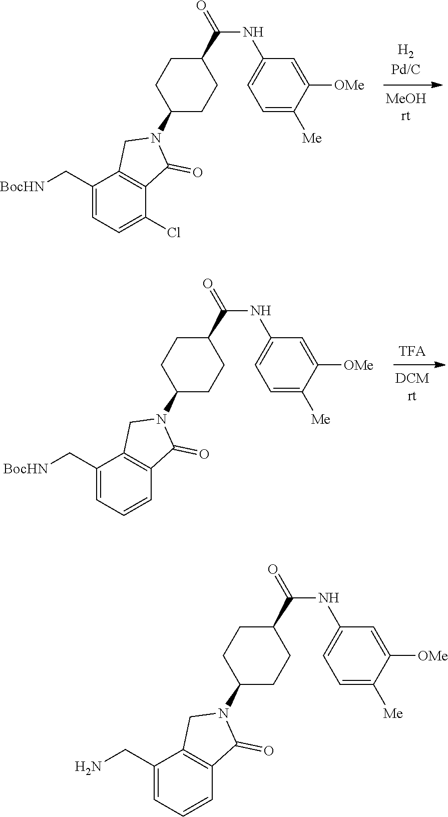 [SMILES: COc1cc(NC(=O)[C@H]2CC[C@@H](N3Cc4c(CNC(=O)OC(C)(C)C)ccc(Cl)c4C3=O)CC2)ccc1C ; COc1cc(NC(=O)[C@H]2CC[C@@H](N3Cc4c(CNC(=O)OC(C)(C)C)cccc4C3=O)CC2)ccc1C ; COc1cc(NC(=O)[C@H]2CC[C@@H](N3Cc4c(CN)cccc4C3=O)CC2)ccc1C]

### Step 1

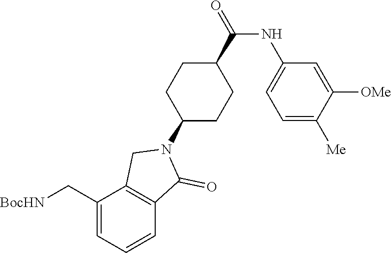 [SMILES: COc1cc(NC(=O)[C@H]2CC[C@@H](N3Cc4c(CNC(=O)OC(C)(C)C)cccc4C3=O)CC2)ccc1C]

### tert-Butyl ((2-((1s,4s)-4-((3-methoxy-4-methylphenyl)carbamoyl)cyclohexyl)-1-oxoisoindolin-4-yl)methyl)carbamate

A solution of tert-butyl ((7-chloro-2-((1s,4s)-4-((3-methoxy-4-methylphenyl)carbamoyl)cyclohexyl)-1-oxoisoindolin-4-yl)methyl)carbamate (35.0 mg, 64.6 μmol) in MeOH (1 mL) was degassed with nitrogen prior to the addition of Palladium/C (6.87 mg, 6.46 μmol). The reaction mixture was capped and purged with hydrogen. The reaction mixture was stirred at room temperature for 3 h under 1 atm of hydrogen (balloon). After full conversion of the starting material, the reaction mixture was purged with nitrogen and filtered using 45 μm filter. The solution was concentrated under reduced pressure and the crude residue was purified by flash chromatography on silica gel (50 g column, from 10 to 80% EtOAc in heptanes) to yield tert-butyl ((2-((1s,4s)-4-((3-methoxy-4-methylphenyl)carbamoyl)cyclohexyl)-1-oxoisoindolin-4-yl)methyl)carbamate (10.9 mg, 33% Yield) as off white solid; LCMS ESI m/z: 508.2 [M+H]⁺.

### Step 2

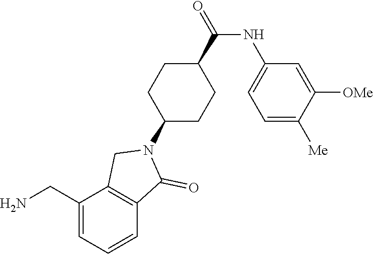 [SMILES: COc1cc(NC(=O)[C@H]2CC[C@@H](N3Cc4c(CN)cccc4C3=O)CC2)ccc1C]

### (1s,4s)-4-(4-(aminomethyl)-1-oxoisoindolin-2-yl)-N-(3-methoxy-4-methylphenyl)cyclohexane-1-carboxamide

To a solution of tert-butyl ((2-((1s,4s)-4-((3-methoxy-4-methylphenyl)carbamoyl)cyclohexyl)-1-oxoisoindolin-4-yl)methyl)carbamate (10.9 mg, 21.5 μmol) in DCM (0.2 mL) was added trifluoroacetic acid (0.2 mL, 2.61 mmol) and the reaction mixture was stirred at room temperature for 1 h. The reaction mixture was concentrated to dryness and the crude residue was purified by reverse phase flash chromatography on C18 (30 g column, from 0 to 60% MeCN in water-10 mM ammonium formate pH=3.8). Pure fractions were directly lyophilized to yield (1s,4s)-4-(4-(aminomethyl)-1-oxoisoindolin-2-yl)-N-(3-methoxy-4-methylphenyl)cyclohexane-1-carboxamide (4.76 mg, 54% Yield) as off white solid; LCMS ESI m/z: 408.4 [M+H]⁺; ¹H NMR (400 MHz, DMSO-d₆) δ 9.72 (s, 1H), 8.30 (s, 1H), 7.56 (t, J=7.0 Hz, 2H), 7.45 (t, J=7.3 Hz, 1H), 7.33 (s, 1H), 7.13 (d, J=8.1 Hz, 1H), 7.02 (d, J=7.8 Hz, 1H), 4.51 (s, 2H), 4.12 (t, J=11.1 Hz, 1H), 3.88 (s, 2H), 3.76 (s, 3H), 2.67 (s, 1H), 2.13 (d, J=13.3 Hz, 2H), 2.08 (s, 3H), 2.02-1.94 (m, 2H), 1.78-1.67 (m, 2H), 1.61 (d, J=10.6 Hz, 2H) (NH₂ signal in line broadening).

### Example 135

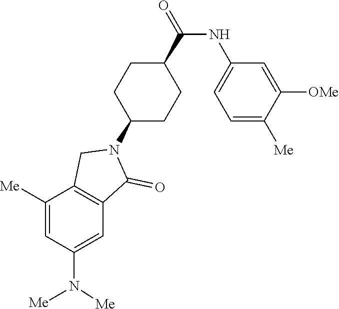 [SMILES: COc1cc(NC(=O)[C@H]2CC[C@@H](N3Cc4c(C)cc(N(C)C)cc4C3=O)CC2)ccc1C]

### (1,4s)-4-(6-(Dimethylamino)-4-methyl-1-oxoisoindolin-2-yl)-N-(3-methoxy-4-methylphenyl)cyclohexane-1-carboxamide

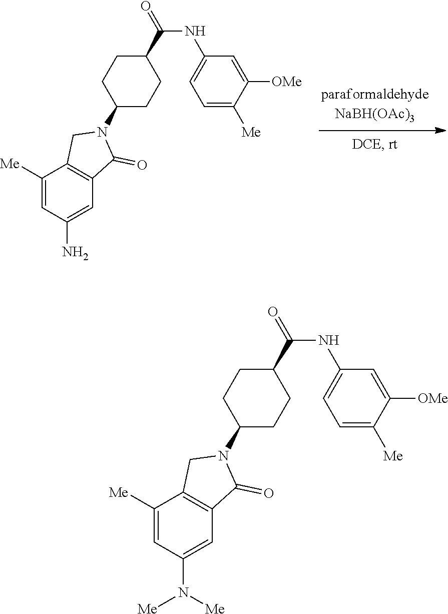 [SMILES: COc1cc(NC(=O)[C@H]2CC[C@@H](N3Cc4c(C)cc(N)cc4C3=O)CC2)ccc1C ; COc1cc(NC(=O)[C@H]2CC[C@@H](N3Cc4c(C)cc(N(C)C)cc4C3=O)CC2)ccc1C]

### Step 1

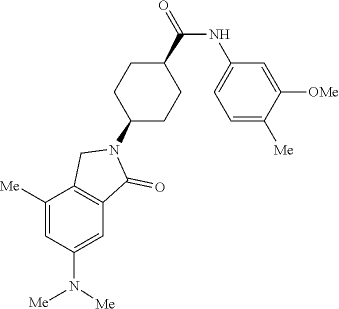 [SMILES: COc1cc(NC(=O)[C@H]2CC[C@@H](N3Cc4c(C)cc(N(C)C)cc4C3=O)CC2)ccc1C]

### (1s,4s)-4-(6-(Dimethylamino)-4-methyl-1-oxoisoindolin-2-yl)-N-(3-methoxy-4-methylphenyl)cyclohexane-1-carboxamide

To a solution of (1s,4s)-4-(6-amino-4-methyl-1-oxoisoindolin-2-yl)-N-(3-methoxy-4-methylphenyl)cyclohexanecarboxamide (1.20 g, 2.94 mmol) in DCE (20 mL) was added paraformaldehyde (243 μL, 8.83 mmol) and sodium triacetoxyborohydride (1.92 g, 8.83 mmol) and the reaction mixture was stirred at 70° C. for 1 h. The reaction mixture was diluted with a saturated solution of sodium bicarbonate and it was extracted 3 times with ethyl acetate. Organics were combined, washed with brine, dried with anhydrous sodium sulfate, filtered and concentrated to dryness. Crude residue was purified by reverse phase flash chromatography on C18 (30 g column, from 2 to 60% MeCN in water-10 mM ammonium formate pH=3.8). Pure fractions were directly lyophilized to yield (1s,4s)-4-(6-(dimethylamino)-4-methyl-1-oxoisoindolin-2-yl)-N-(3-methoxy-4-methylphenyl)cyclohexane-1-carboxamide (298 mg, 23% Yield) as white solid; LCMS ESI m/z: 436.3 [M+H]⁺; ¹H NMR (400 MHz, DMSO-d₆) d 9.71 (s, 1H), 7.30 (d, J=1.5 Hz, 1H), 7.11 (dd, J=8.0, 1.7 Hz, 1H), 7.00 (d, J=8.3 Hz, 1H), 6.76 (d, J=1.6 Hz, 1H), 6.71 (d, J=2.1 Hz, 1H), 4.22 (s, 2H), 4.14-4.02 (m, 1H), 3.74 (s, 3H), 2.89 (s, 6H), 2.67-2.61 (m, 1H), 2.25 (s, 3H), 2.14-2.07 (m, 2H), 2.06 (s, 3H), 2.01-1.88 (m, 2H), 1.76-1.64 (m, 2H), 1.61-1.51 (m, 2H).

### Example 136

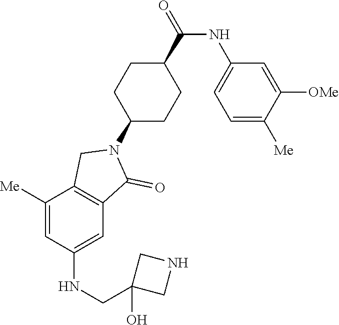 [SMILES: COc1cc(NC(=O)[C@H]2CC[C@@H](N3Cc4c(C)cc(NCC5(O)CNC5)cc4C3=O)CC2)ccc1C]

### (1s,4s)-4-(6-(((3-hydroxyazetidin-3-yl)methyl)amino)-4-methyl-1-oxoisoindolin-2-yl)-N-(3-methoxy-4-methylphenyl)cyclohexanecarboxamide

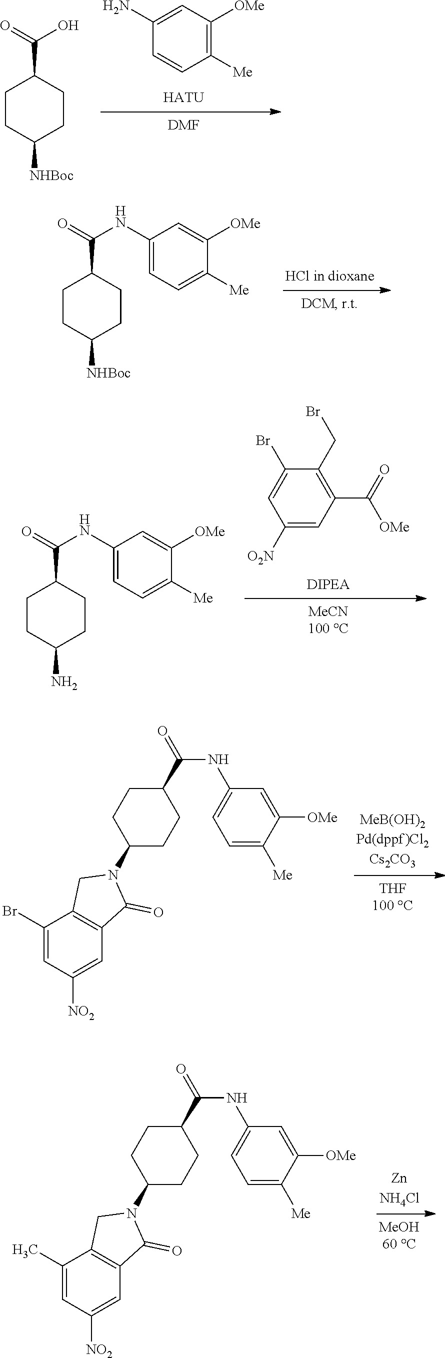

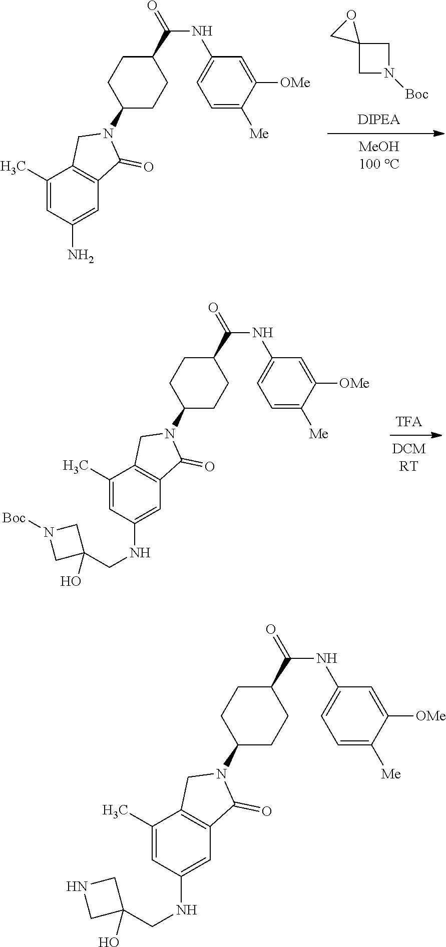 [SMILES: COc1cc(NC(=O)[C@H]2CC[C@@H](N3Cc4c(C)cc(N)cc4C3=O)CC2)ccc1C ; CC(C)(C)OC(=O)N1CC2(CO2)C1 ; COc1cc(NC(=O)[C@H]2CC[C@@H](N3Cc4c(C)cc(NCC5(O)CN(C(=O)OC(C)(C)C)C5)cc4C3=O)CC2)ccc1C ; COc1cc(NC(=O)[C@H]2CC[C@@H](N3Cc4c(C)cc(NCC5(O)CNC5)cc4C3=O)CC2)ccc1C]

### Step 1

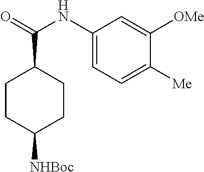 [SMILES: COc1cc(NC(=O)[C@H]2CC[C@@H](NC(=O)OC(C)(C)C)CC2)ccc1C]

### Tert-butyl ((1s,4s)-4-((3-methoxy-4-methylphenyl)carbamoyl)cyclohexyl)carbamate

To a solution of cis-4-(Boc-amino)cyclohexanecarboxylic acid (6.00 g, 24.2 mmol) in DMF (48.3 mL) was added HATU (9.65 g, 25.4 mmol), 3-Methoxy-4-methylaniline (3.38 g, 24.2 mmol) and N,N-Diisopropylethylamine (4.46 mL, 25.4 mmol). The resulting mixture was stirred at room temperature. The reaction mixture was diluted with EtOAc (200 mL). The solution was washed with HCl aq. (50 mL, 1 M) and sat. NaHCO₃ aq. (100 mL) and brine. The organic layer was dried over MgSO₄ and filtered. The solvent was removed under reduced pressure. The residue was saturated with MeOH to provide tert-butyl ((1s,4s)-4-((3-methoxy-4-methylphenyl)carbamoyl)cyclohexyl)carbamate Yield: 76%. LCMS ESI m/z: 263.1 [M-Boc+H].

### Step 2

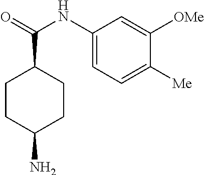 [SMILES: COc1cc(NC(=O)[C@H]2CC[C@@H](N)CC2)ccc1C]

### (1s,4s)-4-amino-N-(3-methoxy-4-methylphenyl)cyclohexanecarboxamide

To a solution of tert-butyl ((1s,4s)-4-((3-methoxy-4-methylphenyl)carbamoyl)cyclohexyl)carbamate (210 mg, 579 μmol) in DCM (1.16 mL) was added hydrochloric acid (724 μL, 2.90 mmol). The resulting mixture was stirred at room temperature. After 30 minutes the solvent was removed under reduced pressure. The residue was suspended in DCM (15 mL) and filtered to afford (1s,4s)-4-amino-N-(3-methoxy-4-methylphenyl)cyclohexanecarboxamide (142 mg, 93%) as a white solid.

### Step 3

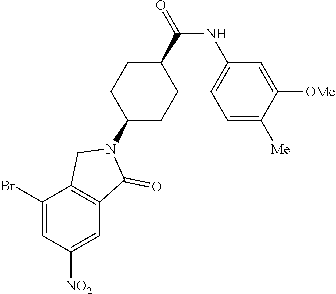 [SMILES: COc1cc(NC(=O)[C@H]2CC[C@@H](N3Cc4c(Br)cc([N+](=O)[O-])cc4C3=O)CC2)ccc1C]

### (1s,4s)-4-(4-bromo-6-nitro-1-oxoisoindolin-2-yl)-N-(3-methoxy-4-methylphenyl)cyclohexanecarboxamide

To a microwave tube was added methyl 3-bromo-2-(bromomethyl)-5-nitrobenzoate (61.0 mg, 233 μmol), (1s,4s)-4-amino-N-(3-methoxy-4-methylphenyl)cyclohexanecarboxamide (82.1 mg, 233 μmol), N, N-diisopropylethylamine (40.5 μL, 230 μmol) and ACN (2.33 mL). The tube was sealed and heated to 100° C. by microwave reactor. After 40 mins the reaction mixture was cooled to room temperature causing the product crashed out. The mixture was filtered to provide (1s,4s)-4-(4-bromo-6-nitro-1-oxoisoindolin-2-yl)-N-(3-methoxy-4-methylphenyl)cyclohexanecarboxamide (88.0 mg, 75%) as a yellow crystal. LCMS ESI m/z: 504.0 [M+H].

### Step 4

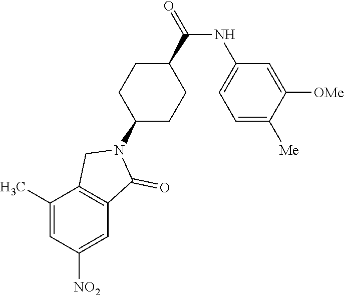 [SMILES: COc1cc(NC(=O)[C@H]2CC[C@@H](N3Cc4c(C)cc([N+](=O)[O-])cc4C3=O)CC2)ccc1C]

### (1s,4s)-N-(3-methoxy-4-methylphenyl)-4-(4-methyl-6-nitro-1-oxoisoindolin-2-yl)cyclohexanecarboxamide

A microwave vial was charged with (1s,4s)-4-(4-bromo-6-nitro-1-oxoisoindolin-2-yl)-N-(3-methoxy-4-methylphenyl)cyclohexanecarboxamide (20.0 mg, 39.8 μmol), 1,1′-bis(diphenylphosphino)ferrocene dichloropalladium (II) (1.49 mg, 1.99 μmol), cesium carbonate (39.7 mg, 119 μmol), methylboronic acid (2.38 mg, 39.8 μmol), THF (1.81 mL) and Water (181 μL). The vial was purged with nitrogen and capped. The reaction mixture was heated to 100° C. After 50 mins the reaction mixture was cooled to room temperature, and diluted with EtOAc (30 mL). This solution was washed with brine, dried over MgSO₄ and filtered. The solvent was removed under reduced pressure to afford (1s,4s)-N-(3-methoxy-4-methylphenyl)-4-(4-methyl-6-nitro-1-oxoisoindolin-2-yl)cyclohexanecarboxamide (10.0 mg, 57%) as a yellow solid. LCMS ESI m/z: 438.2 [M+H].

### Step 5

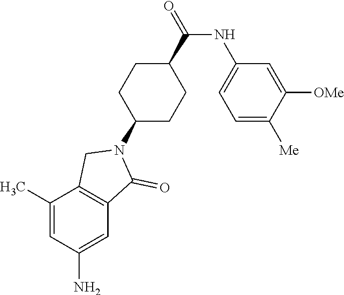 [SMILES: COc1cc(NC(=O)[C@H]2CC[C@@H](N3Cc4c(C)cc(N)cc4C3=O)CC2)ccc1C]

### (1s,4s)-4-(6-amino-4-methyl-1-oxoisoindolin-2-yl)-N-(3-methoxy-4-methylphenyl)cyclohexanecarboxamide

A suspension of (1s,4s)-N-(3-methoxy-4-methylphenyl)-4-(4-methyl-6-nitro-1-oxoisoindolin-2-yl)cyclohexanecarboxamide (100 mg, 229 μmol), zinc (6.30 μL, 686 μmol) and ammonium chloride (36.7 mg, 686 μmol) in MeOH (2.29 mL) was heated to 65° C. After 90 mins, the reaction mixture was cooled to room temperature and diluted with EtOAc (30 mL) and filtered through celite. The solvent was removed under reduced pressure to afford (1s,4s)-4-(6-amino-4-methyl-1-oxoisoindolin-2-yl)-N-(3-methoxy-4-methylphenyl)cyclohexanecarboxamide (71.0 mg, 76%) as a yellow solid. LCMS ESI m/z: 408.3 [M+H].

### Step 6

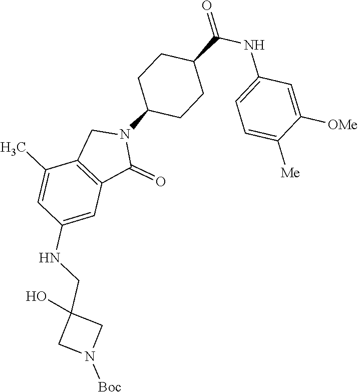 [SMILES: COc1cc(NC(=O)[C@H]2CC[C@@H](N3Cc4c(C)cc(NCC5(O)CN(C(=O)OC(C)(C)C)C5)cc4C3=O)CC2)ccc1C]

### tert-Butyl 3-hydroxy-3-(((2-((1s,4s)-4-((3-methoxy-4-methylphenyl)carbamoyl)cyclohexyl)-7-methyl-3-oxoisoindolin-5-yl)amino)methyl)azetidine-1-carboxylate

To a microwave tube was added (1s,4s)-4-(6-amino-4-methyl-1-oxoisoindolin-2-yl)-N-(3-methoxy-4-methylphenyl)cyclohexanecarboxamide (50.0 mg, 123 μmol), tert-butyl 1-oxa-5-azaspiro[2.3]hexane-5-carboxylate (45.5 mg, 245 μmol), N,N-diisopropylethylamine (42.7 μL, 245 μmol) and MeOH (1.23 mL). The tube was charged with N₂ and capped. The tube was heated to 120° C. using a microwave reactor. After 2 hours, the reaction mixture was cooled to room temperature and reaction mixture was concentrated to dryness under reduced pressure. The crude was purified with flash column chromatography over silica gel using EtOAc (with 10% MeOH additive)/heptane from 0% to 80% to provide tert-butyl 3-hydroxy-3-(((2-((1s,4s)-4-((3-methoxy-4-methylphenyl)carbamoyl)cyclohexyl)-7-methyl-3-oxoisoindolin-5-yl)amino)methyl)azetidine-1-carboxylate (108 mg, 50%) as a colorless liquid. LCMS ESI m/z: 493.2 [M-Boc+H].

### Step 7

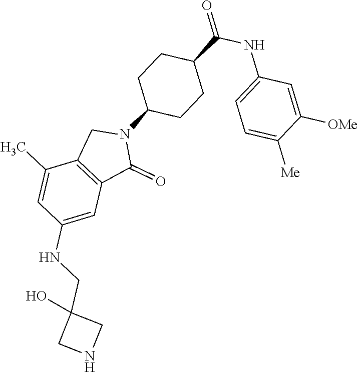 [SMILES: COc1cc(NC(=O)[C@H]2CC[C@@H](N3Cc4c(C)cc(NCC5(O)CNC5)cc4C3=O)CC2)ccc1C]

### (1s,4s)-4-(6-(((3-Hydroxyazetidin-3-yl)methyl)amino)-4-methyl-1-oxoisoindolin-2-yl)-N-(3-methoxy-4-methylphenyl)cyclohexanecarboxamide

A solution of tert-butyl 3-hydroxy-3-(((2-((1s,4s)-4-((3-methoxy-4-methylphenyl)carbamoyl)cyclohexyl)-7-methyl-3-oxoisoindolin-5-yl)amino)methyl)azetidine-1-carboxylate (108 mg, 182 μmol) and trifluoroacetic acid (209 μL, 2.73 mmol) in DCM (1.82 mL) was stirred at room temperature. After 3 hours, the reaction mixture was concentrated under reduced pressure. The residue was purified with reverse phase flash column chromatography using MeCN/AmF from 0% to 40% to afford (1s,4s)-4-(6-(((3-hydroxyazetidin-3-yl)methyl)amino)-4-methyl-1-oxoisoindolin-2-yl)-N-(3-methoxy-4-methylphenyl)cyclohexanecarboxamide (36.0 mg, 40%) as a white solid. LCMS ESI m/z: 493.4 [M+H]. ¹H NMR (400 MHz, DMSO-d₆) δ 9.70 (s, 1H), 8.33 (s, 1H), 7.30 (s, 1H), 7.11 (d, J=8.1 Hz, 1H), 7.00 (d, J=8.1 Hz, 1H), 6.72 (s, 2H), 5.64 (s, 1H), 4.19 (s, 2H), 4.06 (t, J=11.2 Hz, 1H), 3.74 (s, 3H), 3.59 (s, 4H), 3.28 (d, J=5.7 Hz, 2H), 2.67 (d, J=14.0 Hz, 1H), 2.19 (s, 3H), 2.11 (s, 2H), 2.06 (s, 3H), 1.95 (q, J=11.4 Hz, 2H), 1.69 (t, J=13.0 Hz, 2H), 1.56 (d, J=9.2 Hz, 2H) (OH signal in line broadening).

### Example 137

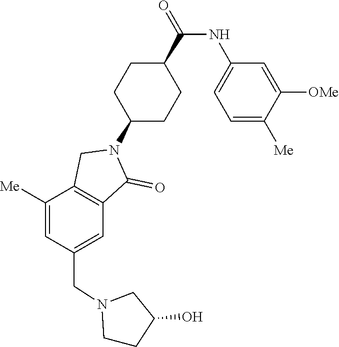 [SMILES: COc1cc(NC(=O)[C@H]2CC[C@@H](N3Cc4c(C)cc(CN5CC[C@@H](O)C5)cc4C3=O)CC2)ccc1C]

### (1S,4s)-4-(6-(((R)-3-Hydroxypyrrolidin-1-yl)methyl)-4-methyl-1-oxoisoindolin-2-yl)-N-(3-methoxy-4-methylphenyl)cyclohexanecarboxamide

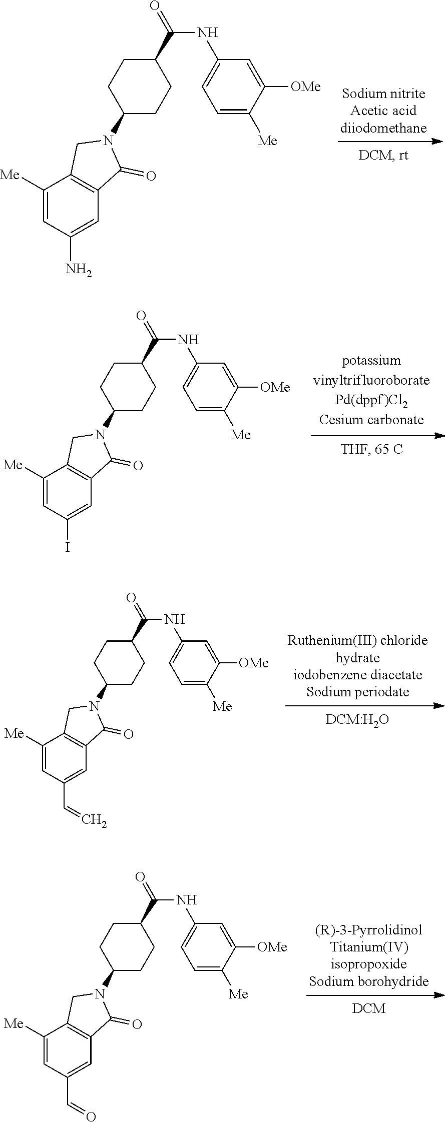 [SMILES: COc1cc(NC(=O)[C@H]2CC[C@@H](N3Cc4c(C)cc(N)cc4C3=O)CC2)ccc1C ; COc1cc(NC(=O)[C@H]2CC[C@@H](N3Cc4c(C)cc(I)cc4C3=O)CC2)ccc1C ; C=Cc1cc(C)c2c(c1)C(=O)N([C@H]1CC[C@@H](C(=O)Nc3ccc(C)c(OC)c3)CC1)C2 ; COc1cc(NC(=O)[C@H]2CC[C@@H](N3Cc4c(C)cc(C=O)cc4C3=O)CC2)ccc1C]

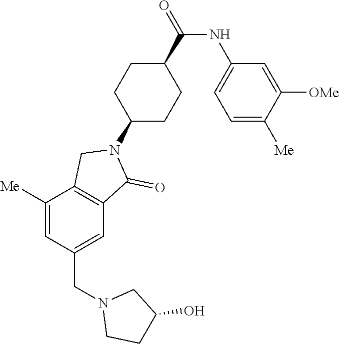 [SMILES: COc1cc(NC(=O)[C@H]2CC[C@@H](N3Cc4c(C)cc(CN5CC[C@@H](O)C5)cc4C3=O)CC2)ccc1C]

### Step 1

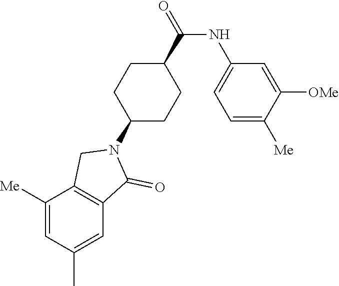 [SMILES: COc1cc(NC(=O)[C@H]2CC[C@@H](N3Cc4c(C)cc(C)cc4C3=O)CC2)ccc1C]

### (1s,4s)-4-(6-Iodo-4-methyl-1-oxoisoindolin-2-yl)-N-(3-methoxy-4-methylphenyl)cyclohexanecarboxamide

To a solution of (1s,4s)-4-(6-amino-4-methyl-1-oxoisoindolin-2-yl)-N-(3-methoxy-4-methylphenyl)cyclohexanecarboxamide (45.0 mg, 110 μmol), sodium nitrite (38.1 mg, 552 μmol), diiodomethane (18.0 μL, 221 μmol) in DCM (2.21 mL) was added acetic acid (127 μL, 2.21 mmol). After 1 hours, the reaction mixture was concentrated under reduced pressure and purified by flash column chromatography over silica gel using EtOAc (with 10% MeOH additive)/Hexane from 0% to 80% to afford (1s,4s)-4-(6-iodo-4-methyl-1-oxoisoindolin-2-yl)-N-(3-methoxy-4-methylphenyl)cyclohexanecarboxamide (45.0 mg, 110 μmol) as a yellow solid. LCMS ESI m/z: 219.0 [M+H].

### Step 2

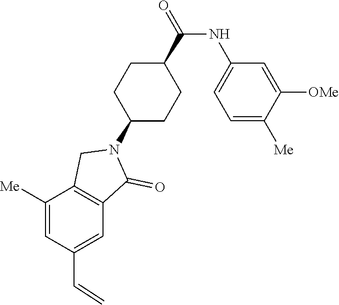 [SMILES: C=Cc1cc(C)c2c(c1)C(=O)N([C@H]1CC[C@@H](C(=O)Nc3ccc(C)c(OC)c3)CC1)C2]

### (1s,4s)-N-(3-Methoxy-4-methylphenyl)-4-(4-methyl-1-oxo-6-vinylisoindolin-2-yl)cyclohexanecarboxamide

To a microwave tube was added (1s,4s)-4-(6-iodo-4-methyl-1-oxoisoindolin-2-yl)-N-(3-methoxy-4-methylphenyl)cyclohexanecarboxamide (100 mg, 193 μmol), 1,1′-Bis(diphenylphosphino)ferrocene dichloropalladium (II) (14.4 mg, 19.3 μmol), Cesium carbonate (192 mg, 579 μmol), potassium vinyltrifluoroborate (29.3 mg, 212 μmol), THF (1.75 mL) and H₂O (175 μL). The tube was charged with N₂ and sealed. The reaction mixture was heated 65° C. After 4 hours, the reaction mixture was concentrated under reduced pressure and purified by flash column chromatography using EtOAc (with 10% MeOH additive)/Hexane from 0% to 80% to afford (1s,4s)-N-(3-methoxy-4-methylphenyl)-4-(4-methyl-1-oxo-6-vinylisoindolin-2-yl)cyclohexanecarboxamide (45.0 mg, 110 μmol) as a yellow solid. LCMS ESI m/z: 419.3 [M+H].

### Step 3

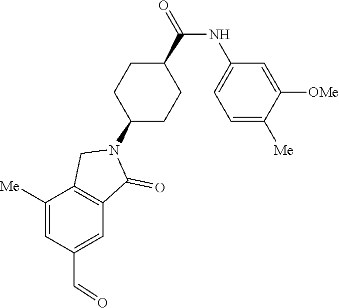 [SMILES: COc1cc(NC(=O)[C@H]2CC[C@@H](N3Cc4c(C)cc(C=O)cc4C3=O)CC2)ccc1C]

### (1s,4s)-4-(6-Formyl-4-methyl-1-oxoisoindolin-2-yl)-N-(3-methoxy-4-methylphenyl)cyclohexanecarboxamide

To a solution of (1s,4s)-N-(3-methoxy-4-methylphenyl)-4-(4-methyl-1-oxo-6-vinylisoindolin-2-yl)cyclohexanecarboxamide (55.0 mg, 131 μmol) in DCM (1.10 mL) and H₂O (219 μL) was added iodobenzene diacetate (130 mg, 394 μmol) and ruthenium (III) chloride hydrate (1.36 mg, 6.57 μmol). After 2 hours, minor conversion was observed. To this mixture was then added sodium periodate (56.8 mg, 263 μmol). After another 2 hours, the reaction mixture was diluted with EtOAc (30 mL). This solution was washed with brine, dried over MgSO₄ and filtered. The solvent was removed under reduced pressure. The residue was purified with flash column chromatography using EtOAc (10% MeOH additive)/Heptane from 0% to 80% to afford (1s,4s)-4-(6-formyl-4-methyl-1-oxoisoindolin-2-yl)-N-(3-methoxy-4-methylphenyl)cyclohexanecarboxamide (12.0 mg, 22%) as a yellow solid. LCMS ESI m/z: 421.2 [M+H].

### Step 4

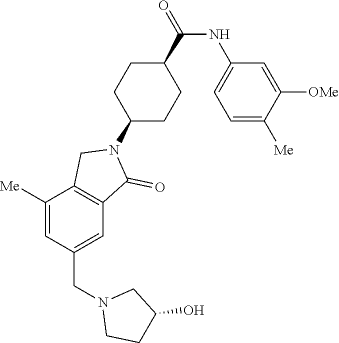 [SMILES: COc1cc(NC(=O)[C@H]2CC[C@@H](N3Cc4c(C)cc(CN5CC[C@@H](O)C5)cc4C3=O)CC2)ccc1C]

### (1s,4s)-4-(6-(((R)-3-Hydroxypyrrolidin-1-yl)methyl)-4-methyl-1-oxoisoindolin-2-yl)-N-(3-methoxy-4-methylphenyl)cyclohexanecarboxamide

To a round bottom flask was added (1s,4s)-4-(6-formyl-4-methyl-1-oxoisoindolin-2-yl)-N-(3-methoxy-4-methylphenyl)cyclohexanecarboxamide (9.00 mg, 21.4 μmol), 3-pyrrolidinol (1.73 μL, 21.4 μmol), titanium (IV) isopropoxide (32.7 μL, 107 μmol) and DCM (1.07 mL). The resulting mixture was stirred at rt. overnight. To this mixture was added sodium borohydride (0.782 μL, 20.5 μmol) and MeOH (0.1 mL). After 30 mins, the reaction mixture was concentrated under reduced pressure. The residue was purified over C18 with ACN/AmF from 0% to 60% to afford (1s,4s)-4-(6-formyl-4-methyl-1-oxoisoindolin-2-yl)-N-(3-methoxy-4 methylphenyl)cyclohexanecarboxamide (4.70 mg, 45%) as a white solid. LCMS ESI m/z: 492.2 [M+H]. ¹H NMR (400 MHz, DMSO-d₆) δ 9.71 (s, 1H), 8.23 (s, 1H), 7.37 (s, 1H), 7.34-7.24 (m, 1H), 7.11 (dd, J=8.0, 1.8 Hz, 1H), 7.00 (d, J=8.1 Hz, 1H), 5.74 (s, 1H), 4.66 (s, 1H), 4.33 (s, 2H), 4.24-4.03 (m, 2H), 3.74 (s, 3H), 3.57 (dd, J=22.2, 13.1 Hz, 3H), 2.62 (dd, J=9.7, 6.3 Hz, 2H), 2.56-2.52 (m, 1H), 2.36 (dd, J=13.9, 8.0 Hz, 1H), 2.30 (s, 3H), 2.26 (dd, J=9.6, 3.7 Hz, 1H), 2.10 (d, J=12.4 Hz, 2H), 2.06 (s, 3H), 2.03-1.93 (m, 3H), 1.70 (ddd, J=13.2, 6.5, 4.1 Hz, 2H), 1.62-1.54 (m, 2H), 1.54-1.44 (m, 1H).

### Example 138

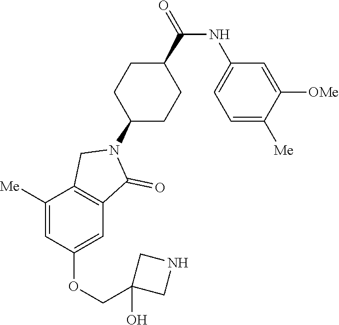 [SMILES: COc1cc(NC(=O)[C@H]2CC[C@@H](N3Cc4c(C)cc(OCC5(O)CNC5)cc4C3=O)CC2)ccc1C]

### 6-(4-(2-Fluoro-5-((4-oxo-7-(prop-1-yn-1-yl)-5-(trifluoromethyl)-3,4-dihydrophthalazin-1-yl)methyl)benzoyl) piperazin-1-yl) nicotinonitrile

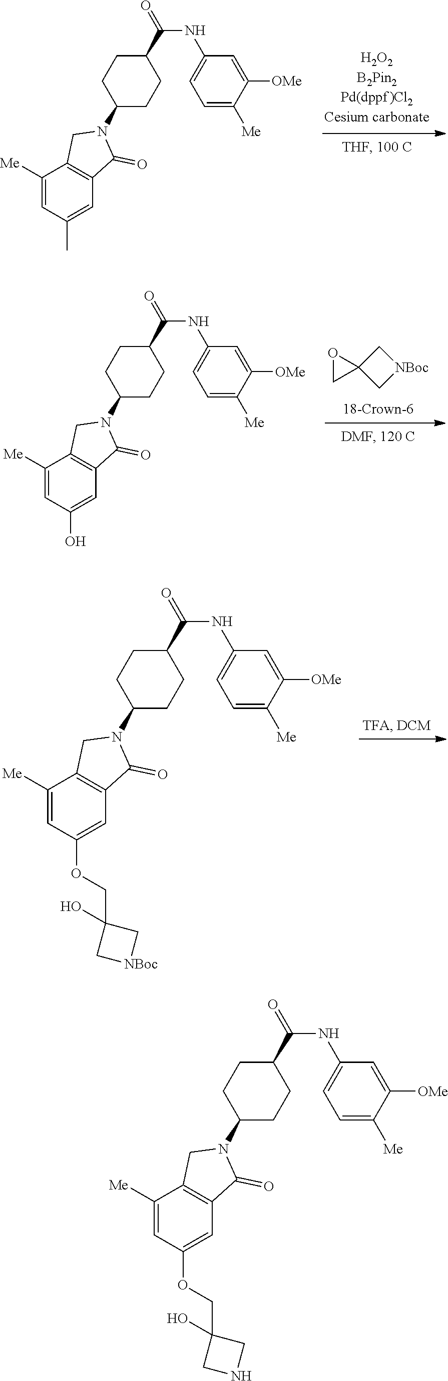 [SMILES: COc1cc(NC(=O)[C@H]2CC[C@@H](N3Cc4c(C)cc(C)cc4C3=O)CC2)ccc1C ; COc1cc(NC(=O)[C@H]2CC[C@@H](N3Cc4c(C)cc(O)cc4C3=O)CC2)ccc1C ; CC(C)(C)OC(=O)N1CC2(CO2)C1 ; COc1cc(NC(=O)[C@H]2CC[C@@H](N3Cc4c(C)cc(OCC5(O)CN(C(=O)OC(C)(C)C)C5)cc4C3=O)CC2)ccc1C ; COc1cc(NC(=O)[C@H]2CC[C@@H](N3Cc4c(C)cc(OCC5(O)CNC5)cc4C3=O)CC2)ccc1C]

### Step 1

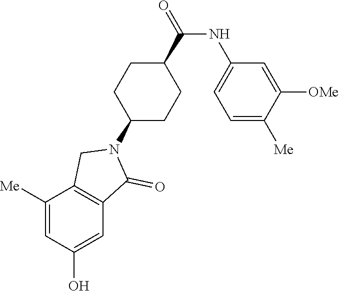 [SMILES: COc1cc(NC(=O)[C@H]2CC[C@@H](N3Cc4c(C)cc(O)cc4C3=O)CC2)ccc1C]

### (1s,4s)-4-(6-Hydroxy-4-methyl-1-oxoisoindolin-2-yl)-N-(3-methoxy-4-methylphenyl)cyclohexanecarboxamide

To a flame-dried round bottom flask was added (1s,4s)-4-(6-iodo-4-methyl-1-oxoisoindolin-2-yl)-N-(3-methoxy-4-methylphenyl)cyclohexanecarboxamide (60.0 mg, 116 μmol), 1,1′-bis(diphenylphosphino)ferrocene dichloropalladium (II) (8.64 mg, 11.6 μmol), bis(pinacolato)diboron (45.5 mg, 174 μmol), cesium carbonate (115 mg, 347 μmol) and THF (1.16 mL). The solvent was charged with nitrogen for 5 minutes. The resulting mixture was heated to reflux. After 3 hours, the reaction mixture was allowed to rt. To this mixture was added hydrogen peroxide (118 μL, 1.16 mmol). After 5 mins, the reaction mixture was diluted with EtOAc (30 mL) and washed with brine. The organic layer was dried over Na₂SO₄ and filtered. The solvent was removed under reduced pressure to afford the crude (1s,4s)-4-(6-hydroxy-4-methyl-1-oxoisoindolin-2-yl)-N-(3-methoxy-4-methylphenyl)cyclohexanecarboxamide (32.0 mg, 68%). LCMS ESI m/z: 409.2 [M+H].

### Step 2

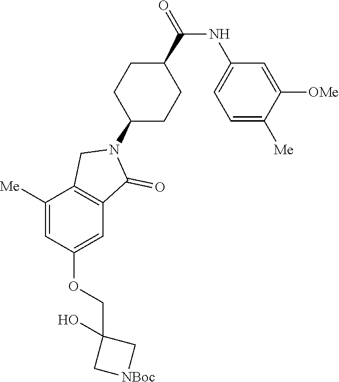 [SMILES: COc1cc(NC(=O)[C@H]2CC[C@@H](N3Cc4c(C)cc(OCC5(O)CN(C(=O)OC(C)(C)C)C5)cc4C3=O)CC2)ccc1C]

### tert-Butyl 3-hydroxy-3-(((2-((1s,4s)-4-((3-methoxy-4-methylphenyl)carbamoyl)cyclohexyl)-7-methyl-3-oxoisoindolin-5-yl)oxy)methyl)azetidine-1-carboxylate

To a microwave tube was added (1s,4s)-4-(6-hydroxy-4-methyl-1-oxoisoindolin-2-yl)-N-(3-methoxy-4-methylphenyl)cyclohexanecarboxamide (32.0 mg, 68%) (30.0 mg, 73.4 μmol), tert-butyl 1-oxa-5-azaspiro[2.3]hexane-5-carboxylate (20.4 mg, 110 μmol), 18-crown-6 (21.6 mg, 80.8 μmol), potassium acetate (8.09 mg, 80.8 μmol), DMF (1.47 mL). The tube was sealed and heated at 120° C. The compound was purified over C18 by reverse phase using ACN/AmF from 0% to 80% to afford tert-butyl 3-hydroxy-3-(((2-((1s,4s)-4-((3-methoxy-4-methylphenyl)carbamoyl)cyclohexyl)-7-methyl-3-oxoisoindolin-5-yl)oxy)methyl)azetidine-1-carboxylate (21.0 mg, 48%) as a white solid. LCMS ESI m/z: 538.1 [M+H].

### Step 3

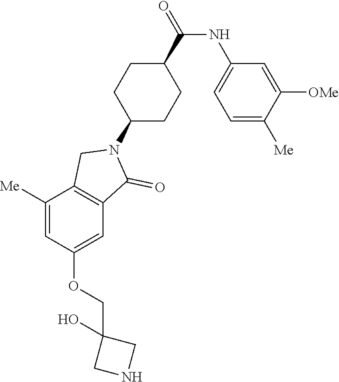 [SMILES: COc1cc(NC(=O)[C@H]2CC[C@@H](N3Cc4c(C)cc(OCC5(O)CNC5)cc4C3=O)CC2)ccc1C]

### (1s,4s)-4-(6-((3-Hydroxyazetidin-3-yl)methoxy)-4-methyl-1-oxoisoindolin-2-yl)-N-(3-methoxy-4-methylphenyl)cyclohexanecarboxamide

A solution of (1s,4s)-4-(6-((3-hydroxyazetidin-3-yl)methoxy)-4-methyl-1-oxoisoindolin-2-yl)-N-(3-methoxy-4-methylphenyl)cyclohexanecarboxamide (7.20 mg, 12.1 μmol) and trifluoroacetic acid (46.4 μL, 606 μmol) in CH₂Cl₂ (1.21 mL) was stirred at r.t. After 90 mins, UPLC-MS confirmed full deprotection. The solvent was removed under reduced pressure. The residue was purified with reverse-phase using ACN/AmF from 0% to 40% to afford (1s,4s)-4-(6-((3-hydroxyazetidin-3-yl)methoxy)-4-methyl-1-oxoisoindolin-2-yl)-N-(3-methoxy-4-methylphenyl)cyclohexanecarboxamide (3.10 mg, 52%) as a white solid. LCMS ESI m/z: 538.1 [M+H]. ¹H NMR (400 MHz, DMSO-d₆) δ 9.71 (s, 1H), 8.28 (s, 1H), 7.30 (d, J=1.6 Hz, 1H), 7.14-7.04 (m, 2H), 7.01-6.99 (m, 2H), 4.29 (s, 2H), 4.08 (s, 4H), 3.82-3.63 (m, 8H), 2.65 (s, 1H), 2.28 (s, 3H), 2.10 (d, J=12.5 Hz, 2H), 2.06 (s, 3H), 2.03-1.88 (m, 2H), 1.77-1.64 (m, 2H), 1.63-1.48 (m, 2H).

### Example 139

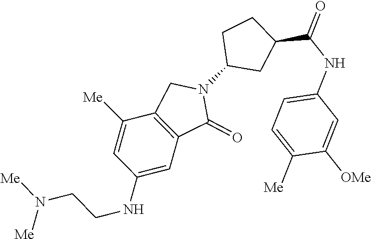 [SMILES: COc1cc(NC(=O)[C@@H]2CC[C@@H](N3Cc4c(C)cc(NCCN(C)C)cc4C3=O)C2)ccc1C]

### 6-(4-(2-Fluoro-5-((4-oxo-7-(prop-1-yn-1-yl)-5-(trifluoromethyl)-3,4-dihydrophthalazin-1-yl)methyl)benzoyl)piperazin-1-yl) nicotinonitrile

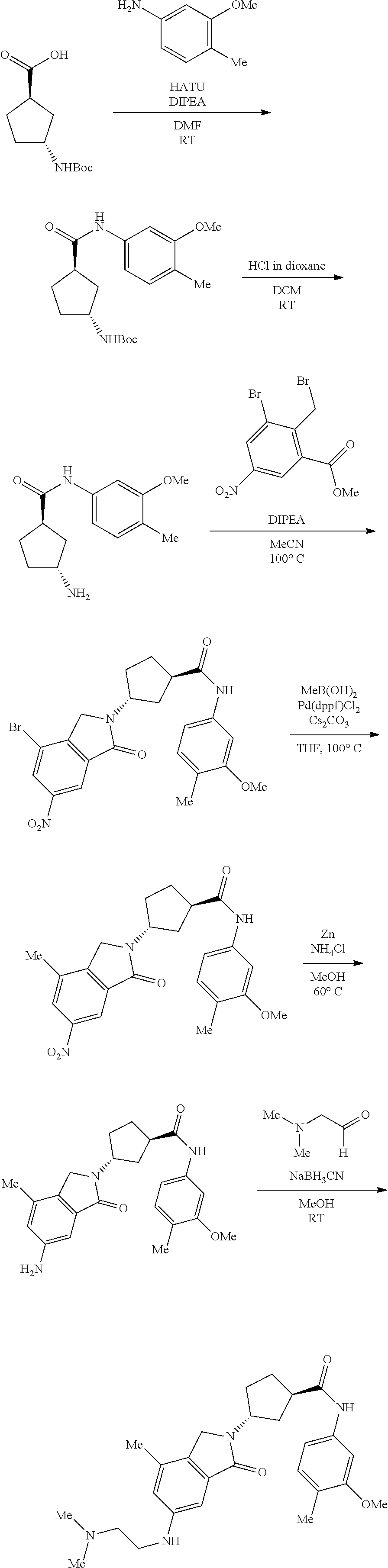

### Step 1

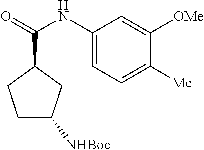 [SMILES: COc1cc(NC(=O)[C@@H]2CC[C@@H](NC(=O)OC(C)(C)C)C2)ccc1C]

### tert-Butyl ((1R,3R)-3-((3-methoxy-4-methylphenyl)carbamoyl)cyclopentyl)carbamate

HATU (528 mg, 1.35 mmol) was added to a stirring solution of (1R,3R)-3-((tert-Butoxycarbonyl)amino)cyclopentanecarboxylic acid (300 mg, 1.28 mmol), 3-Methoxy-4-methylaniline (179 mg, 1.28 mmol) and N,N-diisopropylethylamine (237 μL, 1.35 mmol) in DMF (8.55 mL). The reaction mixture was allowed to stir for 16 h. The reaction was diluted with a saturated solution of NaHCO₃ (50 mL) and extracted into EtOAc (3×50 mL). The organics were combined, washed with H₂O (50 mL), brine (50 mL), dried over Na₂SO₄, filtered and concentrated to a yellow oily solid. The crude material was purified on flash silica column (80 g, dry load, 0-80% EtOAc in Hexanes) to yield the title compound tert-butyl ((1R,3R)-3-((3-methoxy-4-methylphenyl)carbamoyl)cyclopentyl) carbamate (378 mg, 85%) as a white solid. LCMS ESI m/z: 348.4 [M+H].

### Step 2

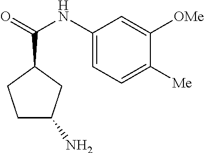 [SMILES: COc1cc(NC(=O)[C@@H]2CC[C@@H](N)C2)ccc1C]

### (1R,3R)-3-Amino-N-(3-methoxy-4-methylphenyl)cyclopentanecarboxamide

To a solution of tert-butyl ((1R,3R)-3-((3-methoxy-4-methylphenyl)carbamoyl)cyclopentyl) carbamate (422 mg, 1.21 mmol) in CH₂Cl₂ (2.42 mL) was added hydrochloric acid (1.51 mL, 6.06 mmol). The resulting mixture was stirred at room temperature. The reaction mixture was allowed to stir overnight. The solvent was removed under reduced pressure to yield the HCl salt as a white solid. The residue was suspended in DCM (50 mL) and neutralized with 1M NaOH to pH 8. The phases were separated and aqeuous was extracted twice more with DCM. The organics were combined, dried over Na₂SO₄, filtered and concentrated to afford (1R,3R)-3-Amino-N-(3-methoxy-4-methylphenyl)cyclopentanecarboxamide (260 mg, 86%) as a a clear oil which solidified upon standing to a white oily solid. LCMS ESI m/z: 247.2 [M+H].

### Step 3

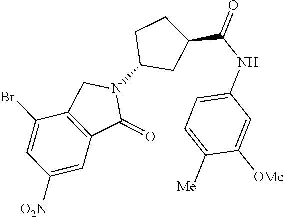 [SMILES: COc1cc(NC(=O)[C@@H]2CC[C@@H](N3Cc4c(Br)cc([N+](=O)[O-])cc4C3=O)C2)ccc1C]

### (1R,3R)-3-(4-Bromo-6-nitro-1-oxoisoindolin-2-yl)-N-(3-methoxy-4-methylphenyl)cyclopentanecarboxamide

To a microwave tube was added methyl 3-bromo-2-(bromomethyl)-5-nitrobenzoate (224 mg, 902 μmol), ACN (1.45 mL), N,N-diisopropylethylamine (26.8 μL, 152 μmol) and (1R,3R)-3-Amino-N-(3-methoxy-4-methylphenyl)cyclopentanecarboxamide (51.2 mg, 145 μmol). The tube was sealed and stirred at room temperature. After 1 hour, the material was dry loaded on silica and purified by flash silica column (60 g, dry load, 0-80% EtOAc in DCM) to yield the title compound (1R,3R)-3-(4-bromo-6-nitro-1-oxoisoindolin-2-yl)-N-(3-methoxy-4-methylphenyl)cyclopentanecarboxamide (353 mg, 80%) as a light yellow solid. LCMS ESI m/z: 489.9 [M+H].

### Step 4

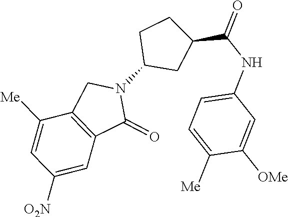 [SMILES: COc1cc(NC(=O)[C@@H]2CC[C@@H](N3Cc4c(C)cc([N+](=O)[O-])cc4C3=O)C2)ccc1C]

### (1R,3R)—N-(3-Methoxy-4-methylphenyl)-3-(4-methyl-6-nitro-1-oxoisoindolin-2-yl)cyclopentanecarboxamide

To a solution of (1R,3R)-3-(4-bromo-6-nitro-1-oxoisoindolin-2-yl)-N-(3-methoxy-4-methylphenyl)cyclopentanecarboxamide (300 mg, 614 μmol) in dioxane (4.29 mL) and water (859 μL) was added potassium carbonate (260 mg, 1.84 mmol) and 1,1′-bis(diphenylphosphino)ferrocene dichloropalladium (II) (47.3 mg, 61.4 μmol) and methylboronic acid (44.1 mg, 737 μmol). The reaction mixture was heated at 100° C. for 7 h. Upon full conversion the reaction mixture was cooled down and evaporated in vacuo. The reaction mixture was cooled down and evaporated in vacuo. The crude material was purified on flash silica (60 g, loaded in DCM SiO₂ precolumn, 0-80% EtOAc in DCM) to yield the title compound (1R,3R)—N-(3-methoxy-4-methylphenyl)-3-(4-methyl-6-nitro-1-oxoisoindolin-2-yl)cyclopentanecarboxamide (162 mg, 55%). LCMS ESI m/z: 424.0 [M+H].

### Step 5

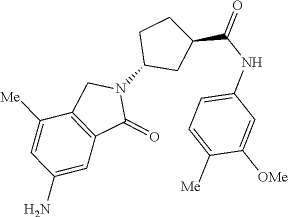 [SMILES: COc1cc(NC(=O)[C@@H]2CC[C@@H](N3Cc4c(C)cc(N)cc4C3=O)C2)ccc1C]

### (1R,3R)-3-(6-Amino-4-methyl-1-oxoisoindolin-2-yl)-N-(3-methoxy-4-methylphenyl)cyclopentanecarboxamide

To a solution of (1R,3R)—N-(3-methoxy-4-methylphenyl)-3-(4-methyl-6-nitro-1-oxoisoindolin-2-yl)cyclopentanecarboxamide (115 mg, 272 μmol) in MeOH (2.16 mL) was added zinc (72.5 mg, 1.09 mmol) and ammonium chloride (58.1 mg, 1.09 mmol). The reaction was left to stir at 60° C. until complete conversion of SM was observed. After complete conversion, the reaction was filtered using a 0.45 μm syringe filter and evaporated under vacuum to afford (1R,3R)-3-(6-amino-4-methyl-1-oxoisoindolin-2-yl)-N-(3-methoxy-4-methylphenyl)cyclopentanecarboxamide (107 mg, 60%) as a yellow oil. LCMS ESI m/z: 394.4 [M+H].

### Step 6

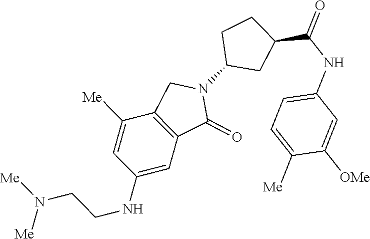 [SMILES: COc1cc(NC(=O)[C@@H]2CC[C@@H](N3Cc4c(C)cc(NCCN(C)C)cc4C3=O)C2)ccc1C]

### (1R,3R)-3-(6-((2-(Dimethylamino)ethyl)amino)-4-methyl-1-oxoisoindolin-2-yl)-N-(3-methoxy-4-methylphenyl)cyclopentanecarboxamide

To a solution of 2-(dimethylamino)acetaldehyde (46.1 mg, 529 μmol) in MeOH was added (1R,3R)-3-(6-amino-4-methyl-1-oxoisoindolin-2-yl)-N-(3-methoxy-4-methylphenyl)cyclopentanecarboxamide (75.0 mg, 191 μmol) and left to stir for 1 hour. Then, sodium cyanoborohydride (32.3 μL, 529 μmol) was quickly added and the reaction left to stir. Upon completion, the reaction mixture was concentrated under vacuum. The residue was dissolved in DMSO and subjected to reverse phase chromatography (5-95% AmF: MeCN over 30 minutes) through wet loading. The fractions containing compound were pooled together and lyophilized to afford (1R,3R)-3-(6-((2-(dimethylamino)ethyl)amino)-4-methyl-1-oxoisoindolin-2-yl)-N-(3-methoxy-4-methylphenyl)cyclopentanecarboxamide (15.0 mg, 18%) as a white powder. LCMS ESI m/z: 465.4 [M+H]. ¹H NMR (400 MHz, DMSO-d₆) δ 9.88 (s, 1H), 9.50 (s, 1H), 7.36 (d, J=1.2 Hz, 1H), 7.04 (m, 2H) 6.73 (s, 1H), 6.68 (s, 1H), 4.74 (p, J=8.0 Hz, 1H), 4.30 (s, 2H), 3.75 (s, 3H), 3.42 (t, J=6.1 Hz, 2H), 3.21 (t, J=6.1 Hz, 2H), 3.06 (dd, J=14.7, 7.7 Hz, 1H), 2.82 (s, 6H), 2.25 (s, 3H), 2.08 (s, 3H), 2.10-2.00 (m, 2H), 2.01-1.91 (m, 2H), 1.79 (q, J=10.7 Hz, 2H).

### Example 140

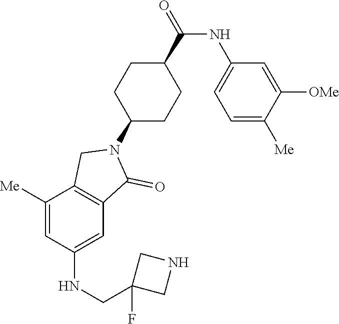 [SMILES: COc1cc(NC(=O)[C@H]2CC[C@@H](N3Cc4c(C)cc(NCC5(F)CNC5)cc4C3=O)CC2)ccc1C]

### 6-(4-(2-Fluoro-5-((4-oxo-7-(prop-1-yn-1-yl)-5-(trifluoromethyl)-3,4-dihydrophthalazin-1-yl)methyl)benzoyl)piperazin-1-yl)nicotinonitrile

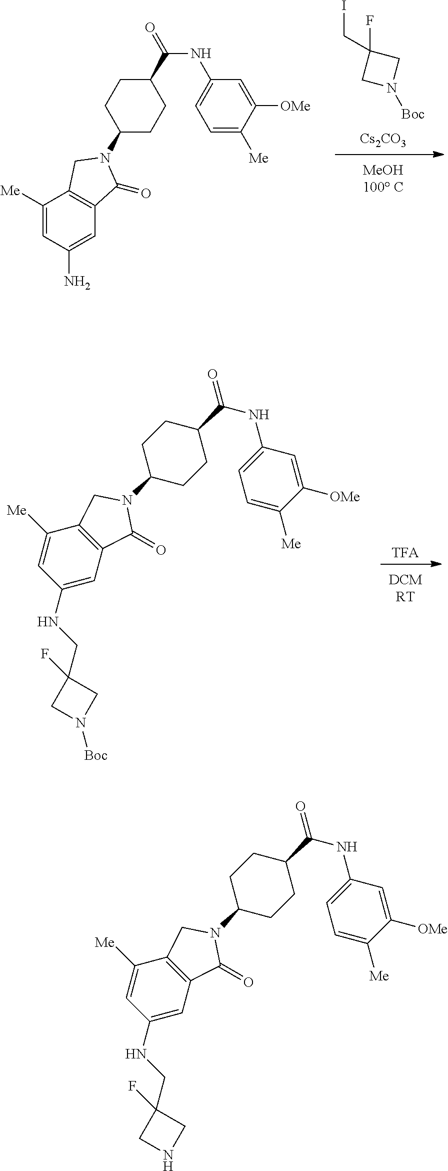

### Step 1

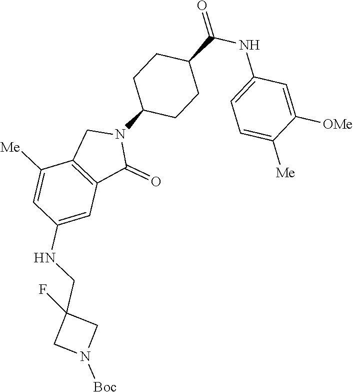 [SMILES: COc1cc(NC(=O)[C@H]2CC[C@@H](N3Cc4c(C)cc(NCC5(F)CN(C(=O)OC(C)(C)C)C5)cc4C3=O)CC2)ccc1C]

### tert-Butyl 3-fluoro-3-(((2-((1s,4s)-4-((3-methoxy-4-methylphenyl)carbamoyl)cyclohexyl)-7-methyl-3-oxoisoindolin-5-yl)amino)methyl)azetidine-1-carboxylate

To a microwave tube was added (1s,4s)-4-(6-amino-4-methyl-1-oxoisoindolin-2-yl)-N-(3-methoxy-4-methylphenyl)cyclohexanecarboxamide, and intermediate from the synthesis of Example 136, (23.0 mg, 56.3 μmol), tert-butyl 3-fluoro-3-(iodomethyl)azetidine-1-carboxylate (26.6 mg, 84.5 μmol), cesium carbonate (18.5 mg, 56.3 μmol), DMF (1.41 mL). The tube was sealed and heated to 120° C. by a microwave reactor. After 1 hour, the reaction mixture was allowed to room temperature. The reaction mixture was purified by reverse phase chromatography using ACN/AmF from 0% to 70% to afford tert-butyl 3-fluoro-3-(((2-((1s,4s)-4-((3-methoxy-4-methylphenyl)carbamoyl)cyclohexyl)-7-methyl-3-oxoisoindolin-5-yl)amino)methyl)azetidine-1-carboxylate (18.0 mg, 54%) as a white solid. LCMS ESI m/z: 540.4 [M-tBu+H].

### Step 2

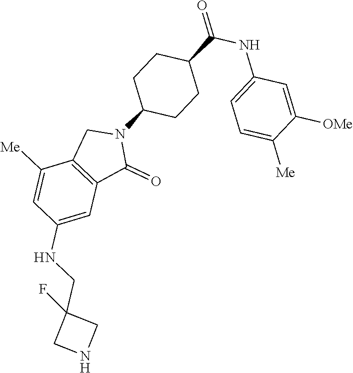 [SMILES: COc1cc(NC(=O)[C@H]2CC[C@@H](N3Cc4c(C)cc(NCC5(F)CNC5)cc4C3=O)CC2)ccc1C]

### (1s,4s)-4-(6-(((3-Fluoroazetidin-3-yl)methyl)amino)-4-methyl-1-oxoisoindolin-2-yl)-N-(3-methoxy-4-methylphenyl)cyclohexanecarboxamide

A solution of tert-butyl 3-fluoro-3-(((2-((1s,4s)-4-((3-methoxy-4-methylphenyl)carbamoyl)cyclohexyl)-7-methyl-3-oxoisoindolin-5-yl)amino)methyl)azetidine-1-carboxylate (18.0 mg, 30.3 μmol) and trifluoroacetic acid (46.8 μL, 605 μmol) in CH₂Cl₂ (1.51 mL) was stirred at rt. After 2 hours, the solvent was removed under reduced pressure. The residue was purified with reverse-phase chromatography over C18 using ACN/AmF from 0% to 60% to afford (1s,4s)-4-(6-(((3-fluoroazetidin-3-yl)methyl)amino)-4-methyl-1-oxoisoindolin-2-yl)-N-(3-methoxy-4-methylphenyl)cyclohexanecarboxamide (10.2 mg, 68%) as a white solid. LCMS ESI m/z: 495.5 [M+H]. ¹H NMR (400 MHz, DMSO-d₆) δ 9.70 (s, 1H), 8.19 (s, 1H), 7.30 (d, J=1.6 Hz, 1H), 7.10 (dd, J=8.0, 1.8 Hz, 1H), 7.00 (d, J=8.1 Hz, 1H), 6.72 (d, J=12.3 Hz, 2H), 5.97 (t, J=6.1 Hz, 1H), 4.19 (s, 2H), 4.05 (tt, J=10.9, 3.3 Hz, 1H), 3.75 (s, 3H), 3.68 (dd, J=19.9, 9.9 Hz, 2H), 3.61-3.53 (m, 4H), 3.49 (d, J=6.1 Hz, 1H), 2.65 (s, 1H), 2.19 (s, 3H), 2.11 (s, 2H), 2.06 (s, 3H), 2.00-1.86 (m, 2H), 1.76-1.62 (m, 2H), 1.61-1.50 (m, 2H).

### Example 141

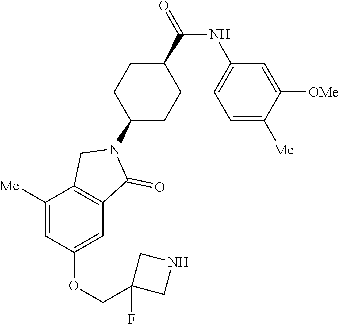 [SMILES: COc1cc(NC(=O)[C@H]2CC[C@@H](N3Cc4c(C)cc(OCC5(F)CNC5)cc4C3=O)CC2)ccc1C]

### (1s,4s)-4-(6-((3-Fluoroazetidin-3-yl)methoxy)-4-methyl-1-oxoisoindolin-2-yl)-N-(3-methoxy-4-methylphenyl)cyclohexanecarboxamide

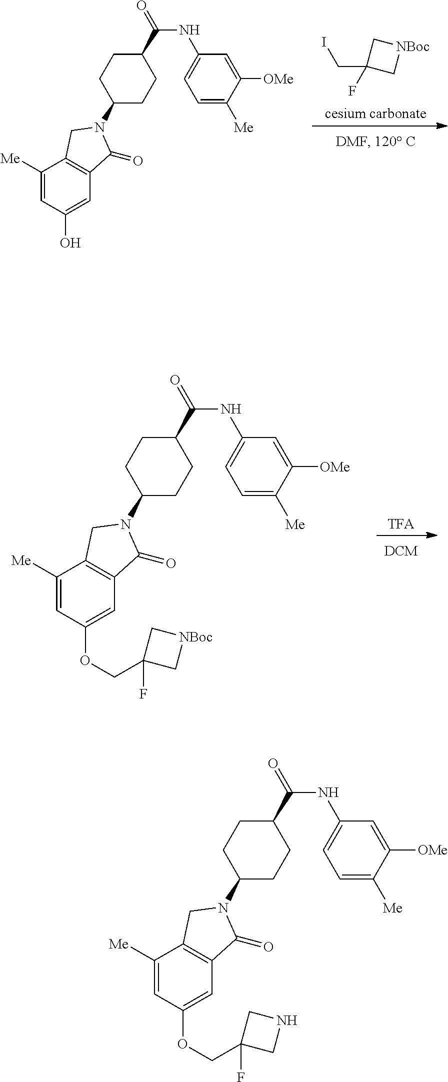

### Step 1

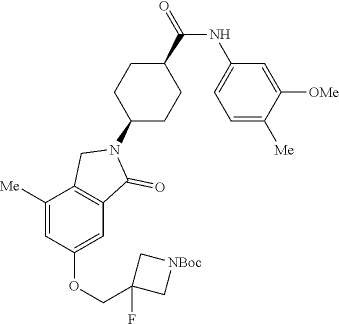 [SMILES: COc1cc(NC(=O)[C@H]2CC[C@@H](N3Cc4c(C)cc(OCC5(F)CN(C(=O)OC(C)(C)C)C5)cc4C3=O)CC2)ccc1C]

### tert-Butyl 3-fluoro-3-(((2-((1s,4s)-4-((3-methoxy-4-methylphenyl)carbamoyl)cyclohexyl)-7-methyl-3-oxoisoindolin-5-yl)oxy)methyl)azetidine-1-carboxylate

To a microwave tube was added (1s,4s)-4-(6-hydroxy-4-methyl-1-oxoisoindolin-2-yl)-N-(3-methoxy-4-methylphenyl)cyclohexanecarboxamide, an intermediate from the synthesis of Example 138, (23.0 mg, 56.3 μmol), tert-butyl 3-fluoro-3-(iodomethyl)azetidine-1-carboxylate (26.6 mg, 84.5 μmol), cesium carbonate (18.5 mg, 56.3 μmol), DMF (1.41 mL). The tube was sealed and heated to 120° C. by a microwave reactor. After 1 hour, the reaction mixture was allowed to room temperature. The reaction mixture was purified by reverse phase chromatography using C18 and ACN/AmF from 0% to 70% to afford tert-butyl 3-fluoro-3-(((2-((1s,4s)-4-((3-methoxy-4-methylphenyl)carbamoyl)cyclohexyl)-7-methyl-3-oxoisoindolin-5-yl)oxy)methyl)azetidine-1-carboxylate (18.0 mg, 54%) as a white solid. LCMS ESI m/z: 540.4 [M+H].

### Step 2

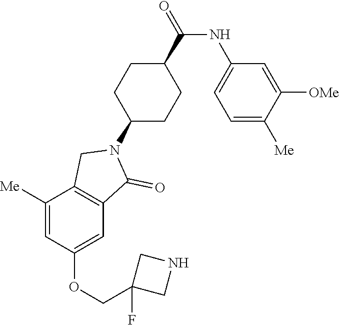 [SMILES: COc1cc(NC(=O)[C@H]2CC[C@@H](N3Cc4c(C)cc(OCC5(F)CNC5)cc4C3=O)CC2)ccc1C]

### (1s,4s)-4-(6-((3-Fluoroazetidin-3-yl)methoxy)-4-methyl-1-oxoisoindolin-2-yl)-N-(3-methoxy-4-methylphenyl)cyclohexanecarboxamide

A solution of tert-butyl 3-fluoro-3-(((2-((1s,4s)-4-((3-methoxy-4-methylphenyl)carbamoyl)cyclohexyl)-7-methyl-3-oxoisoindolin-5-yl)oxy)methyl)azetidine-1-carboxylate (16.0 mg, 26.9 μmol) and trifluoroacetic acid (30.9 μL, 403 μmol) in CH₂Cl₂ (269 μL) was stirred at room temperature. After 1 hour, the solvent was removed under reduced pressure. The residue was purified with ACN/AmF from 0% to 60% to afford (1s,4s)-4-(6-((3-fluoroazetidin-3-yl)methoxy)-4-methyl-1-oxoisoindolin-2-yl)-N-(3-methoxy-4-methylphenyl)cyclohexanecarboxamide (6.00 mg, 45%) as a white solid. LCMS ESI m/z: 496.4 [M+H]. ¹H NMR (400 MHz, DMSO-d₆) δ 9.71 (s, 1H), 8.20 (s, 1H), 7.31 (d, J=1.4 Hz, 1H), 7.11 (dd, J=8.0, 1.5 Hz, 1H), 7.06 (d, J=1.9 Hz, 1H), 7.01-6.99 (m, 2H), 4.39 (s, 1H), 4.33 (s, 1H), 4.30 (s, 2H), 4.15-4.02 (m, 2H), 3.74 (s, 3H), 3.72-3.63 (m, 4H), 2.65 (s, 1H), 2.28 (s, 3H), 2.10 (d, J=14.5 Hz, 2H), 2.06 (s, 3H), 1.97 (dd, J=23.9, 12.3 Hz, 2H), 1.77-1.64 (m, 2H), 1.64-1.53 (m, 2H).

### Example 142

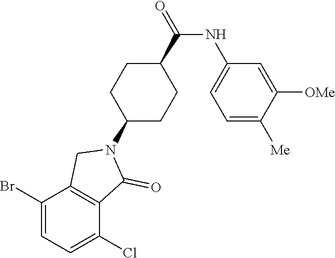 [SMILES: COc1cc(NC(=O)[C@H]2CC[C@@H](N3Cc4c(Br)ccc(Cl)c4C3=O)CC2)ccc1C]

### (1s,4s)-4-(4-Bromo-7-chloro-1-oxoisoindolin-2-yl)-N-(3-methoxy-4-methylphenyl)cyclohexanecarboxamide

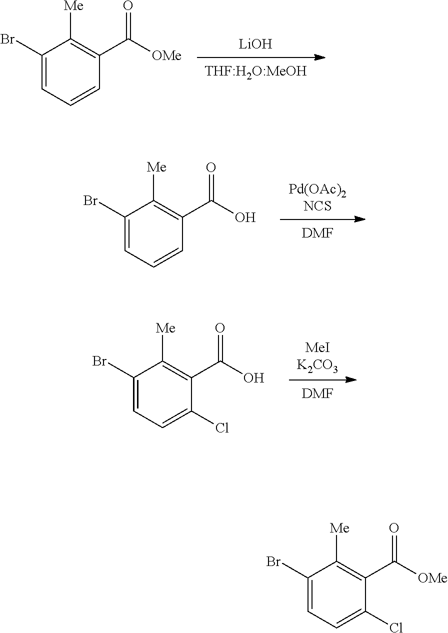

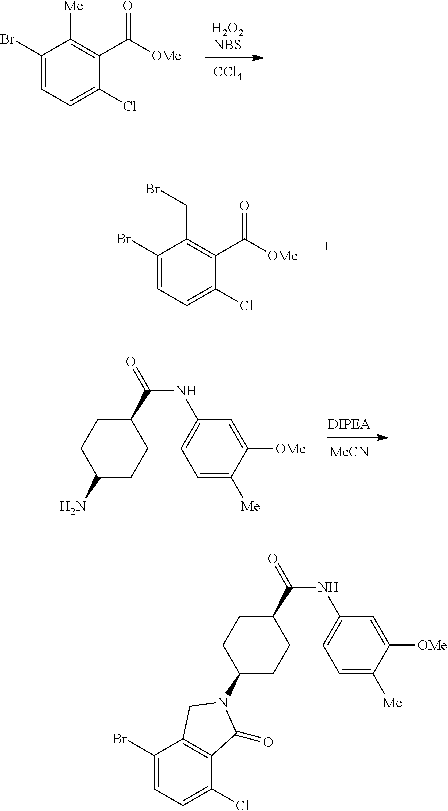

### Step 1

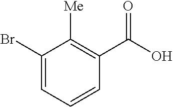 [SMILES: Cc1c(Br)cccc1C(=O)O]

### 3-Bromo-2-methylbenzoic Acid

To a stirred solution of methyl 3-bromo-2-methylbenzoate (3.57 mL, 21.4 mmol) in THF (50 mL) was added lithium hydroxide (5.23 g, 214 mmol) followed by H₂O (4.99 mL) and MeOH (4.99 mL) at 0° C. The mixture was warmed to room temperature slowly and stirred for 2 hours. Additional eq. of LiOH were needed until 10 eq. was reached. The reaction was then concentrated under reduced pressure. Then, 2 M HCl and EtOAc were added and the mixture was extracted x3 using EtOAc and washed with brine. The organic layer was then concentrated to afford 3-bromo-2-methylbenzoic acid (4.50 g, 98%) as a off-white solid. LCMS ESI m/z: 214.9 [M−H].

### Step 2

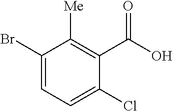 [SMILES: Cc1c(Br)ccc(Cl)c1C(=O)O]

### 3-Bromo-6-chloro-2-methylbenzoic Acid

To a solution of 3-bromo-2-methylbenzoic acid (4.71 mL, 21.9 mmol) in DMF (51.2 mL) were added N-chlorosuccinimide (3.58 g, 26.3 mmol) and palladium (II) acetate (2.51 g, 10.9 mmol). The solution was left to stir at 110° C. for 16 h under a nitrogen atmosphere. After complete conversion was observed by LCMS, to the reaction was added 1:1 EtOAc:Water, and the aqueous layer was extracted ×3 using EtOAc. The combined organic layers were then washed with brine x3, and finally concentrated. The remaining residue was dissolved in DMSO and subjected to reverse phase chromatography using C18 and a 5-95% AmF-MeCN gradient over 20 minutes. The pooled fractions were concentrated under vacuum to afford 3-bromo-6-chloro-2-methylbenzoic acid (3.00 g, 54%) as a red oil. LCMS ESI m/z: 249.0 [M−H].

### Step 3

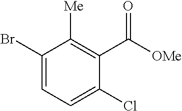 [SMILES: COC(=O)c1c(Cl)ccc(Br)c1C]

### Methyl 3-bromo-6-chloro-2-methylbenzoate

To a solution of 3-Bromo-6-chloro-2-methylbenzoic acid (30.0 mg, 120 μmol) in DMF (1.20 mL) was added potassium carbonate (41.5 mg, 301 μmol). After stirring at room temperature for 30 min, iodomethane (9.07 μL, 144 μmol) was added. After stirring for 16 hr at room temperature, the reaction was diluted with dilute HCl and extracted by EtOAc 3x. The combined organic layer was dried over sodium sulfate, filtered and concentrated to afford methyl 3-bromo-6-chloro-2-methylbenzoate (1.58 mg, 5%). LCMS ESI m/z: No ionization.

### Step 4

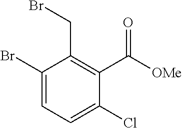 [SMILES: COC(=O)c1c(Cl)ccc(Br)c1CBr]

### Methyl 3-bromo-2-(bromomethyl)-6-chlorobenzoate

To a solution of methyl 3-bromo-2-(bromomethyl)-6-chlorobenzoate (1.50 g, 5.69 mmol) in CCl₄ (28.0 mL) was added n-bromosuccinimide (1.02 g, 5.69 mmol) and finally Luperox A98, benzoyl peroxide, >98% (60.6 μL, 285 μmol). The solution was refluxed for 16 hours. The solvent was then removed under vacuum and the crude was dissolved in minimal DCM and wet-loaded onto a normal phase silica gel column (20 g). The product was purified using Heptanes-EtOAc (20%-100%). The fractions containing product were pooled to afford methyl 3-bromo-2-(bromomethyl)-6-chlorobenzoate (1.46 g, 75%) as a yellow oil. ¹H NMR (400 MHz, DMSO-d₆) δ 7.85 (d, J=8.7 Hz, 1H), 7.54 (d, J=8.7 Hz, 1H), 4.58 (s, 2H), 3.95 (s, 3H).

### Step 5

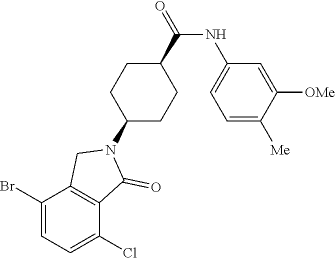 [SMILES: COc1cc(NC(=O)[C@H]2CC[C@@H](N3Cc4c(Br)ccc(Cl)c4C3=O)CC2)ccc1C]

### (1s,4s)-4-(4-Bromo-7-chloro-1-oxoisoindolin-2-yl)-N-(3-methoxy-4-methylphenyl)cyclohexanecarboxamide

To a solution of methyl 3-bromo-2-(bromomethyl)-6-chlorobenzoate (1.46 g, 4.26 mmol) in ACN (71.1 mL) was added (1r,4r)-4-amino-N-(3-methoxy-4-methylphenyl)cyclohexanecarboxamide (1.23 g, 4.69 mmol) and N,N-Diisopropylethylamine (1.12 mL, 6.40 mmol). The solution was left to stir. After complete conversion was observed, the reaction mixture was concentrated under vacuum. The resulting residue was dissolved in DCM and wet loaded onto a normal phase column (40 g) and purified using Heptane:EtOAc (20%-95%). The fractions containing product were pooled and rotavaped to afford (1r,4r)-4-(4-bromo-7-chloro-1-oxoisoindolin-2-yl)-N-(3-methoxy-4-methylphenyl)cyclohexanecarboxamide (1.50 g, 72%) as a light brown solid. LCMS ESI m/z: 491.0 [M−H]. NMR: ¹H NMR (400 MHz, DMSO-d₆) δ 9.74 (s, 1H), 7.78 (d, J=8.4 Hz, 1H), 7.46 (d, J=8.4 Hz, 1H), 7.33 (d, J=1.6 Hz, 1H), 7.11 (dd, J=8.0, 1.8 Hz, 1H), 7.02 (d, J=8.2 Hz, 1H), 4.35 (s, 2H), 4.13-4.04 (m, 1H), 3.76 (s, 3H), 2.66 (s, 1H), 2.14-2.05 (m, 5H), 2.03-1.93 (m, 2H), 1.72 (dd, J=16.0, 7.6 Hz, 2H), 1.68-1.58 (m, 2H).

### Example 143

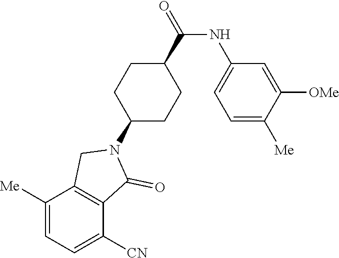 [SMILES: COc1cc(NC(=O)[C@H]2CC[C@@H](N3Cc4c(C)ccc(C#N)c4C3=O)CC2)ccc1C]

### (1s,4s)-4-(7-Cyano-4-methyl-1-oxoisoindolin-2-yl)-N-(3-methoxy-4-methylphenyl)cyclohexanecarboxamide

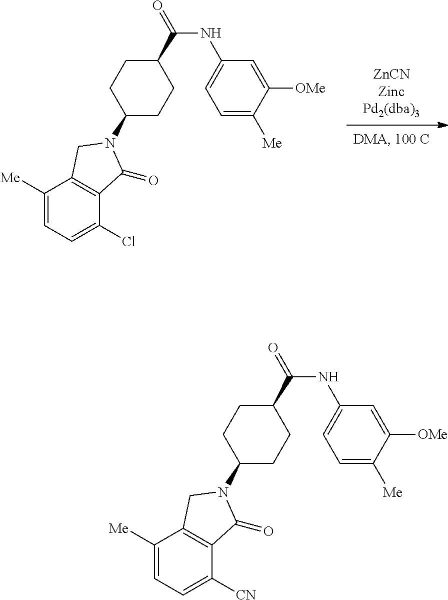 [SMILES: COc1cc(NC(=O)[C@H]2CC[C@@H](N3Cc4c(C)ccc(Cl)c4C3=O)CC2)ccc1C ; "DMA, 100 C" ; "ZnCNZincPd2(dba)3" ; COc1cc(NC(=O)[C@H]2CC[C@@H](N3Cc4c(C)ccc(C#N)c4C3=O)CC2)ccc1C]

### Step 1

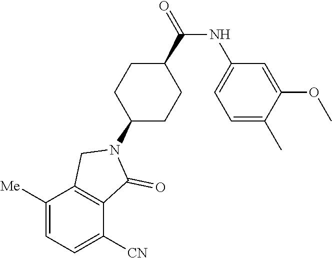 [SMILES: COc1cc(NC(=O)[C@H]2CC[C@@H](N3Cc4c(C)ccc(C#N)c4C3=O)CC2)ccc1C]

### (1s,4s)-4-(7-Cyano-4-methyl-1-oxoisoindolin-2-yl)-N-(3-methoxy-4-methylphenyl)cyclohexanecarboxamide

To a solution of (1s,4s)-4-(7-chloro-4-methyl-1-oxoisoindolin-2-yl)-N-(3-methoxy-4-methylphenyl)cyclohexanecarboxamide (100 mg, 234 μmol) in degassed DMA (4.68 mL) in an inert heat-dried reaction vessel was added 2-dicyclohexylphosphino-2′,4′,6′-triisopropylbiphenyl (25.3 mg, 51.5 μmol), tris(dibenzylideneacetone)-dipalladium (0) (24.3 mg, 25.8 μmol), zinc (1.6 mg, 25.8 μmol) and zinc cyanide (28.6 mg, 246 μmol) in sequential order. The reaction was heated to 105° C. under inert atmosphere. After completion of starting material, the reaction was filtered through a 0.45 μm syringe filter. The solvent was suspended in water and EtOAc and the solution was extracted x3 with EtOAc. The organic layers were pooled and dried under vacuum. The residue was suspended in minimal DCM and wet loaded onto a 60 g silica gel column and purified using Heptanes: EtOAc (20-95%). The fractions containing product were pooled and dried under vacuum to afford (1s,4s)-4-(7-cyano-4-methyl-1-oxoisoindolin-2-yl)-N-(3-methoxy-4-methylphenyl)cyclohexanecarboxamide (50.9 mg, 52%) as a yellow powder. LCMS ESI m/z: 427.2 [M+H]. ¹H NMR (400 MHz, DMSO-d₆) δ 9.72 (s, 1H), 7.84 (d, J=7.7 Hz, 1H), 7.58-7.54 (m, 1H), 7.33 (d, J=1.7 Hz, 1H), 7.13 (dd, J=8.0, 1.8 Hz, 1H), 7.02 (d, J=8.3 Hz, 1H), 4.48 (s, 2H), 4.12 (ddd, J=11.1, 7.6, 3.9 Hz, 1H), 3.76 (s, 3H), 2.68 (s, 1H), 2.42 (s, 3H), 2.13-2.07 (m, 5H), 2.04-1.95 (m, 2H), 1.74 (dd, J=17.2, 8.6 Hz, 2H), 1.65 (dd, J=8.6, 3.7 Hz, 2H).

### Example 144

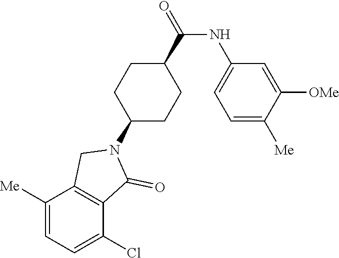 [SMILES: COc1cc(NC(=O)[C@H]2CC[C@@H](N3Cc4c(C)ccc(Cl)c4C3=O)CC2)ccc1C]

### (1s,4s)-4-(7-Chloro-4-methyl-1-oxoisoindolin-2-yl)-N-(3-methoxy-4-methylphenyl)cyclohexanecarboxamide

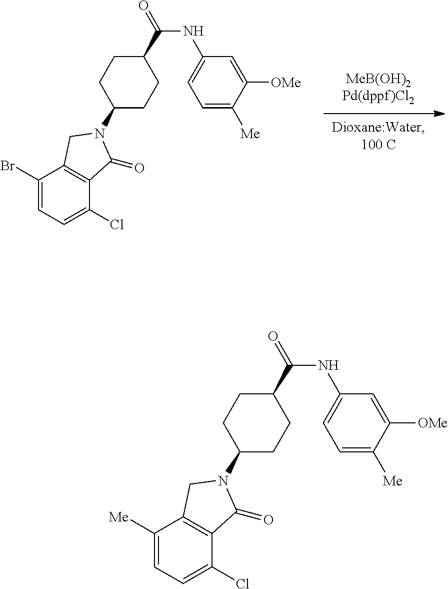 [SMILES: COc1cc(NC(=O)[C@H]2CC[C@@H](N3Cc4c(Br)ccc(Cl)c4C3=O)CC2)ccc1C ; "Dioxane:Water,100 C" ; "MeB(OH)2Pd(dppf)Cl2" ; COc1cc(NC(=O)[C@H]2CC[C@@H](N3Cc4c(C)ccc(Cl)c4C3=O)CC2)ccc1C]

### Step 1

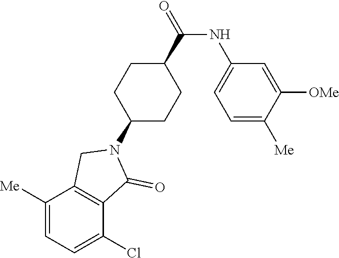 [SMILES: COc1cc(NC(=O)[C@H]2CC[C@@H](N3Cc4c(C)ccc(Cl)c4C3=O)CC2)ccc1C]

### (1s,4s)-4-(7-Chloro-4-methyl-1-oxoisoindolin-2-yl)-N-(3-methoxy-4-methylphenyl)cyclohexanecarboxamide

To a solution of (1s,4s)-4-(7-chloro-4-methyl-1-oxoisoindolin-2-yl)-N-(3-methoxy-4-methylphenyl)cyclohexanecarboxamide (1.20 g, 2.44 mmol) in dioxane (34.6 mL) and water (6.93 mL) was added potassium carbonate (1.01 g, 7.32 mmol) and 1,1′-bis(diphenylphosphino)ferrocene dichloropalladium (II) (268 mg, 366 μmol). The reaction mixture was heated at 100° C. for 5 hours. After complete conversion, the mixture was filtered through a 0.45 μm filter and the solvent was concentrated under vacuum to afford a oily residue. The residue was dissolved in minimal DCM and wet loaded onto a 60 g normal phase silica gel column and purified using 25-100% Heptanes: EtOAc. The fractions containing product were pooled to afford the product as a pink solid (~700 mg). The compound was then columned again using reverse phase chromotography over C18 (wet loaded with DMSO, 5-50% then 50-95% AmF:MeCN). The fractions containing product were pooled and concentrated under vacuum to afford (1s,4s)-4-(7-chloro-4-methyl-1-oxoisoindolin-2-yl)-N-(3-methoxy-4-methylphenyl)cyclohexanecarboxamide (625 mg, 60%) as a white solid. LCMS ESI m/z: 427.2 [M+H]. ¹H NMR (400 MHz, DMSO-d₆) δ 9.71 (s, 1H), 7.36-7.31 (m, 3H), 7.13 (dd, J=8.0, 1.8 Hz, 1H), 7.02 (d, J=8.2 Hz, 1H), 4.37 (s, 2H), 4.09 (ddd, J=11.2, 7.6, 3.9 Hz, 1H), 3.76 (s, 3H), 2.67 (s, 1H), 2.30 (s, 3H), 2.14 (s, 1H), 2.11 (s, 1H), 2.08 (m, 3H), 2.05-1.92 (m, 2H), 1.73 (m, 2H), 1.66-1.57 (m, 2H).

### Example 145

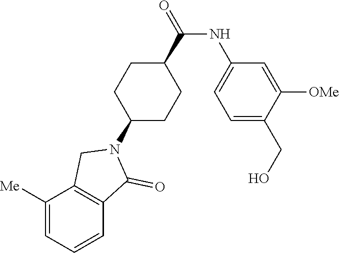 [SMILES: COc1cc(NC(=O)[C@H]2CC[C@@H](N3Cc4c(C)cccc4C3=O)CC2)ccc1CO]

### (1s,4s)-N-(4-(Hydroxymethyl)-3-methoxyphenyl)-4-(4-methyl-1-oxoisoindolin-2-yl)cyclohexanecarboxamide

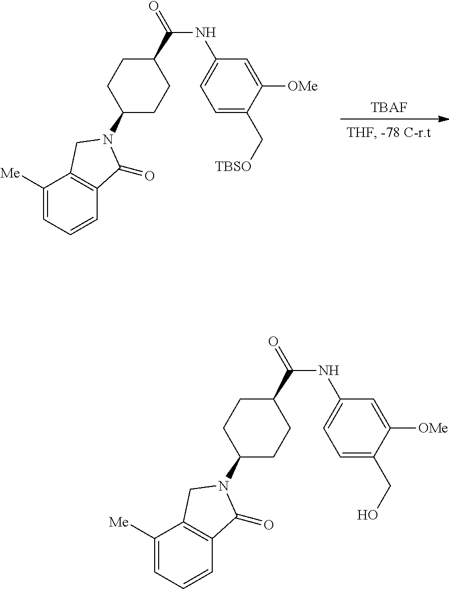 [SMILES: COc1cc(NC(=O)[C@H]2CC[C@@H](N3Cc4c(C)cccc4C3=O)CC2)ccc1CO[Si](C)(C)C(C)(C)C ; "TBAF" ; "THF, -78 C-r.t" ; COc1cc(NC(=O)[C@H]2CC[C@@H](N3Cc4c(C)cccc4C3=O)CC2)ccc1CO]

### Step 1

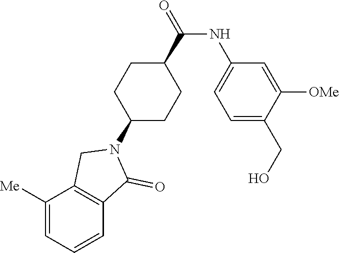 [SMILES: COc1cc(NC(=O)[C@H]2CC[C@@H](N3Cc4c(C)cccc4C3=O)CC2)ccc1CO]

### (1s,4s)-N-(4-(Hydroxymethyl)-3-methoxyphenyl)-4-(4-methyl-1-oxoisoindolin-2-yl)cyclohexanecarboxamide

To a vial with (1s,4s)-N-(4-(((tert-butyldimethylsilyl)oxy)methyl)-3-methoxyphenyl)-4-(4-methyl-1-oxoisoindolin-2-yl)cyclohexanecarboxamide (10.0 mg, 19.1 μmol) was added THF (500 μL) and tetrabutylammonium fluoride 1.0 M in THF (57.4 μL, 57.4 μmol). The resulting mixture was stirred at −78° C. for 1 h. The reaction was then quenched with sat. NaHCO₃ and extracted with EtOAc (×3) then dried over Na₂SO₄ and concentrated under reduced pressure.

The crude product was then purified using reverse phase column chromatography on a C18 10 g column from 0 to 80% ACN: AMF (10 mM). The product was concentrated and lyophilized to afford a pale yellow powder (1.42 mg, 18%). LCMS ESI m/z: 409.5 [M+H]. ¹H NMR (400 MHz, DMSO-d₆) δ 9.79 (s, 1H), 7.53-7.45 (m, 1H), 7.41-7.33 (m, 1H), 7.22 (q, J=8.2 Hz, 2H), 4.90 (d, J=5.5 Hz, 1H), 4.55-4.34 (m, 4H), 4.12 (s, 1H), 3.75 (s, 3H), 2.69 (s, 1H), 2.54 (s, 1H), 2.34 (s, 3H), 2.11 (t, J=12.5 Hz, 2H), 1.99 (t, J=11.8 Hz, 2H), 1.74 (d, J=12.8 Hz, 2H), 1.61 (d, J=9.2 Hz, 3H).

### Example 146

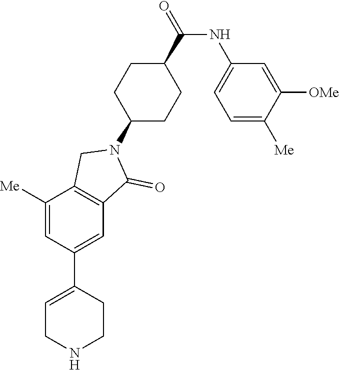 [SMILES: COc1cc(NC(=O)[C@H]2CC[C@@H](N3Cc4c(C)cc(C5=CCNCC5)cc4C3=O)CC2)ccc1C]

### (1s,4s)-N-(3-Methoxy-4-methylphenyl)-4-(4-methyl-1-oxo-6-(1,2,3,6-tetrahydropyridin-4-yl)isoindolin-2-yl)cyclohexanecarboxamide

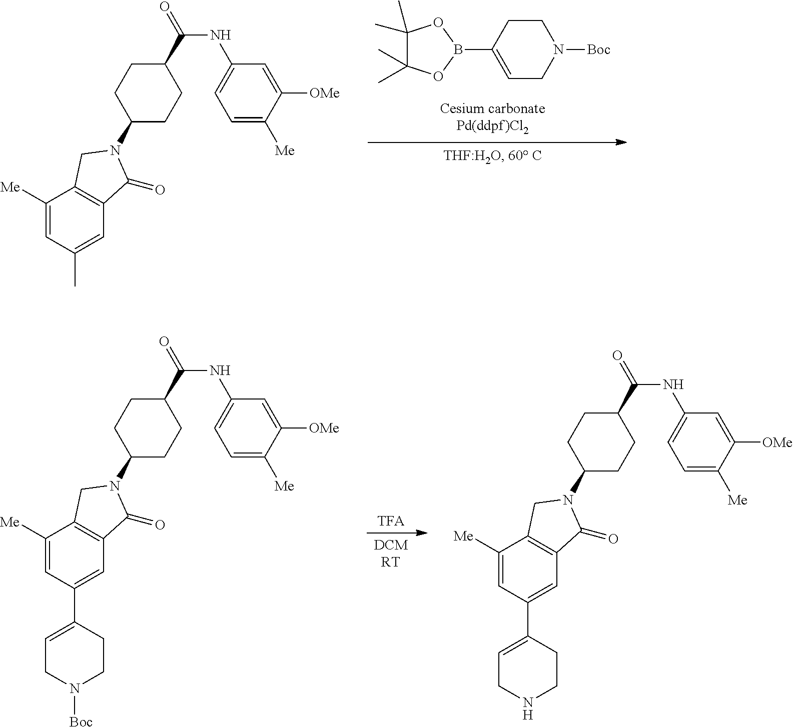

### Step 1

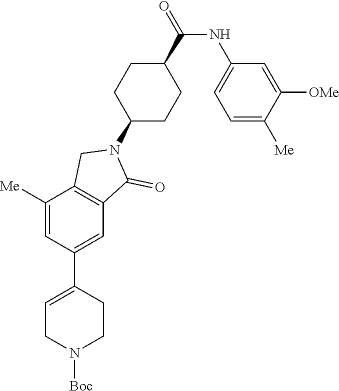 [SMILES: COc1cc(NC(=O)[C@H]2CC[C@@H](N3Cc4c(C)cc(C5=CCN(C(=O)OC(C)(C)C)CC5)cc4C3=O)CC2)ccc1C]

### tert-Butyl 4-(2-((1,4s)-4-((3-methoxy-4-methylphenyl)carbamoyl)cyclohexyl)-7-methyl-3-oxoisoindolin-5-yl)-5,6-dihydropyridine-1 (2H)-carboxylate

A solution of (1s,4s)-4-(6-iodo-4-methyl-1-oxoisoindolin-2-yl)-N-(3-methoxy-4-methylphenyl)cyclohexanecarboxamide, an intermediate from the synthesis of Example 137, (56.0 mg, 108 μmol), (1-(tert-butoxycarbonyl)-1,2,3,6-tetrahydropyridin-4-yl) boronic acid (24.5 mg, 103 μmol), cesium carbonate (108 mg, 324 μmol), 1,1′-bis(diphenylphosphino)ferrocene dichloropalladium (II) (8.07 mg, 10.8 μmol) in THF (2.70 mL)/H₂O (270 μL) stirred at 60° C. under N₂. After 8 hours, the reaction mixture was diluted with EtOAc and washed with brine. The organic phase was dried over Na₂SO₄ and filtered. The solvent was removed under reduced pressure to afford tert-butyl 4-(2-((1s,4s)-4-((3-methoxy-4-methylphenyl)carbamoyl)cyclohexyl)-7-methyl-3-oxoisoindolin-5-yl)-5,6-dihydropyridine-1 (2H)-carboxylate (46.0 mg, 74%) as a pale yellow solid. LCMS ESI m/z: 574.3.

### Step 2

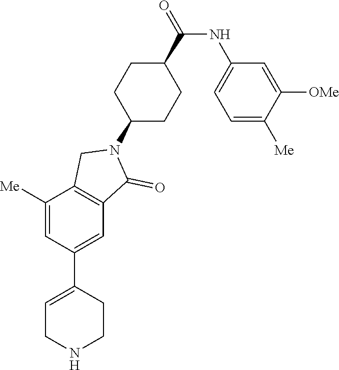 [SMILES: COc1cc(NC(=O)[C@H]2CC[C@@H](N3Cc4c(C)cc(C5=CCNCC5)cc4C3=O)CC2)ccc1C]

### (1s,4s)-4-(6-Formyl-4-methyl-1-oxoisoindolin-2-yl)-N-(3-methoxy-4-methylphenyl)cyclohexanecarboxamide

A solution of tert-butyl 4-(2-((1s,4s)-4-((3-methoxy-4-methylphenyl)carbamoyl)cyclohexyl)-7-methyl-3-oxoisoindolin-5-yl)-5,6-dihydropyridine-1(2H)-carboxylate (30.0 mg, 52.3 μmol) and trifluoroacetic acid (80.9 μL, 1.05 mmol) in DCM (1.05 mL) was stirred at rt. After 30 mins, the reaction mixture was diluted with EtOAc and washed with brine. The organic phase was dried over Na₂SO₄ and filtered. The solvent was removed under reduced pressure. The residue was purified with reverse phase using C18 and ACN/AmF from 0% to 50% to afford (1s,4s)-N-(3-methoxy-4-methylphenyl)-4-(4-methyl-1-oxo-6-(1,2,3,6-tetrahydropyridin-4-yl)isoindolin-2-yl)cyclohexanecarboxamide (46.0 mg, 74%) as a beige solid. LCMS ESI m/z: 474.1. ¹H NMR (400 MHz, DMSO-d₆) δ 9.72 (s, 1H), 8.30 (s, 1H), 7.46 (d, J=2.5 Hz, 2H), 7.31 (d, J=1.6 Hz, 1H), 7.11 (dd, J=8.0, 1.7 Hz, 1H), 7.00 (d, J=8.3 Hz, 1H), 6.25 (s, 1H), 4.37 (s, 2H), 4.10 (t, J=11.1 Hz, 1H), 3.74 (s, 3H), 3.55 (s, 2H), 3.10 (t, J=5.2 Hz, 2H), 2.66 (s, 1H), 2.52 (s, 2H), 2.33 (s, 3H), 2.11 (d, J=12.8 Hz, 2H), 2.06 (s, 3H), 2.04-1.90 (m, 3H), 1.71 (dd, J=17.2, 8.5 Hz, 2H), 1.64-1.53 (m, 2H).

### Example 147

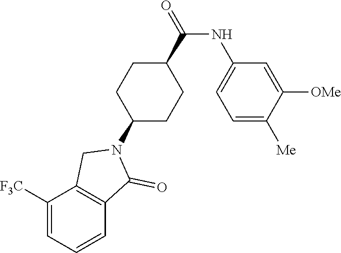 [SMILES: COc1cc(NC(=O)[C@H]2CC[C@@H](N3Cc4c(cccc4C(F)(F)F)C3=O)CC2)ccc1C]

### (1s,4s)-N-(3-Methoxy-4-methylphenyl)-4-(1-oxo-4-(trifluoromethyl)isoindolin-2-yl)cyclohexanecarboxamide

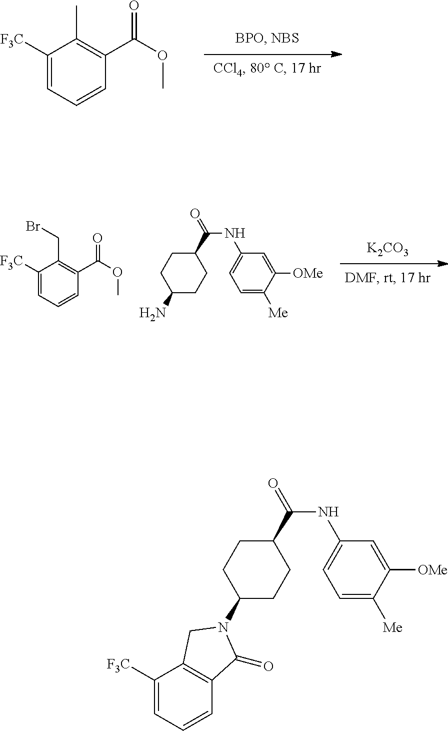

### Step 1

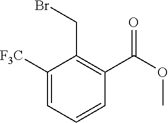 [SMILES: COC(=O)c1cccc(C(F)(F)F)c1CBr]

### Methyl 2-(bromomethyl)-3-(trifluoromethyl)benzoate

To a solution of methyl 2-methyl-3-(trifluoromethyl)benzoate (100 mg, 459 mmol) in CCl₄ (1 mL) was added 1-bromopyrrolidine-2,5-dione (90 mg, 500 mmol, 43 μL) and benzoyl benzenecarboperoxoate (111 mg, 458 mmol). The mixture was stirred at 80° C. for 17 hours. The reaction mixture was concentrated and purified by silica gel chromatography (PE/EtOAc=40/1 to 20/1) to give methyl 2-(bromomethyl)-3-(trifluoromethyl)benzoate (50 mg, 170 mmol, 37% yield) as a yellow oil. ¹H NMR (400 MHz, DMSO-d₆) δ8.10 (d, J=7.7 Hz, 1H), 7.99 (d, J=7.9 Hz, 1H), 7.70 (t, J=7.9 Hz, 1H), 5.04 (s, 2H), 3.93 (s, 3H).

### Step 2

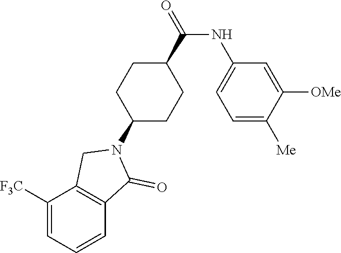 [SMILES: COc1cc(NC(=O)[C@H]2CC[C@@H](N3Cc4c(cccc4C(F)(F)F)C3=O)CC2)ccc1C]

### (1s,4s)-N-(3-Methoxy-4-methylphenyl)-4-(1-oxo-4-(trifluoromethyl)isoindolin-2-yl)cyclohexanecarboxamide

To a solution of methyl 2-(bromomethyl)-3-(trifluoromethyl)benzoate (50 mg, 170 mmol) and 4-amino-N-(3-methoxy-4-methyl-phenyl)cyclohexanecarboxamide (44 mg, 170 mmol) in DMF (1 mL) was added K₂CO₃ (70 mg, 500 mmol). The mixture was stirred at 25° C. for 17 hours. The mixture was poured into EtOAc (20 mL) and washed with water (10 mL*3). The residue was purified by prep-HPLC to give (1s,4s)-N-(3-methoxy-4-methylphenyl)-4-(1-oxo-4-(trifluoromethyl)isoindolin-2-yl)cyclohexanecarboxamide (2.6 mg, 5.8 mmol, 3.5% yield) as a white solid. LCMS ESI m/z 446.9 [M+H]⁺. ¹HNMR (400 MHz, DMSO-d₆) δ 9.72 (s, 1H), 7.99-7.93 (m, 2H), 7.73 (t, J=7.6 Hz, 1H), 7.32 (d, J=1.4 Hz, 1H), 7.11 (d, J=8.0 Hz, 1H), 7.03 (d, J=8.0 Hz, 1H), 4.62 (s, 2H), 4.22-4.02 (m, 1H), 3.76 (s, 3H), 2.68-2.66 (m, 1H), 2.19-1.93 (m, 7H), 1.83-1.56 (m, 4H).

### Example 148

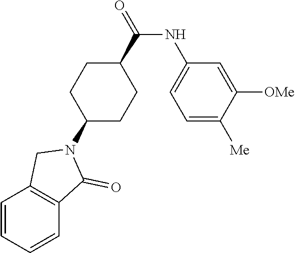 [SMILES: COc1cc(NC(=O)[C@H]2CC[C@@H](N3Cc4ccccc4C3=O)CC2)ccc1C]

### (1s,4s)-N-(3-Methoxy-4-methylphenyl)-4-(1-oxoisoindolin-2-yl)cyclohexanecarboxamide

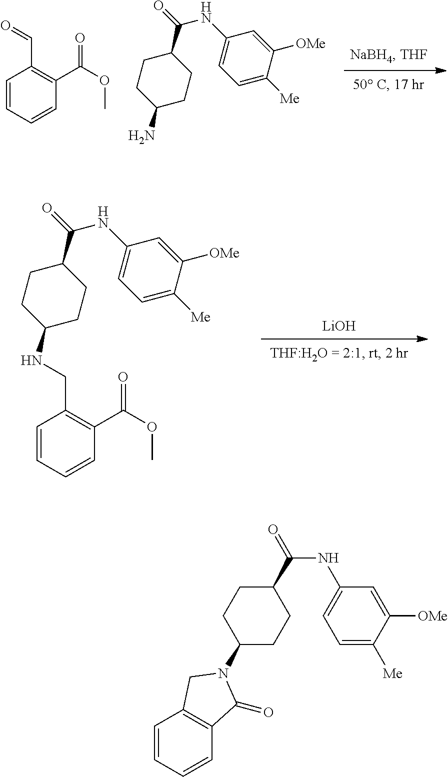

### Step 1

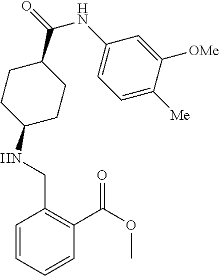 [SMILES: COC(=O)c1ccccc1CN[C@H]1CC[C@@H](C(=O)Nc2ccc(C)c(OC)c2)CC1]

### Methyl 2-(((1s,4s)-4-(3-methoxy-4-methylphenylcarbamoyl)cyclohexylamino)methyl)benzoate

A solution of methyl 2-formylbenzoate (500 mg, 3.05 mmol) and 4-amino-N-(3-methoxy-4-methyl-phenyl)cyclohexanecarboxamide (799 mg, 3.05 mmol) in THF (2 mL) was stirred at 50° C. for 1 hour. The reaction mixture was cooled to room temperature, and NaBH₄ (346 mg, 9.14 mmol) was added to the mixture. The resulting mixture was stirred at room temperature for 16 hours. The mixture was concentrated and purified by purified by silica gel column (PE:EtOAc=30:1 to 1:1) to give methyl 2-(((1s,4s)-4-(3-methoxy-4-methylphenylcarbamoyl)cyclohexylamino)methyl)benzoate (500 mg, 1.2 mmol, 40% yield) as a white solid. LCMS ESI m/z 410.9 [M+H]⁺.

### Step 2

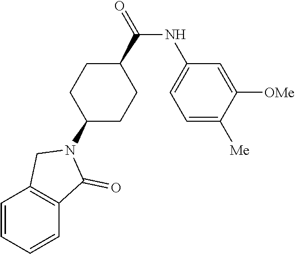 [SMILES: COc1cc(NC(=O)[C@H]2CC[C@@H](N3Cc4ccccc4C3=O)CC2)ccc1C]

### (1s,4s)-N-(3-Methoxy-4-methylphenyl)-4-(1-oxoisoindolin-2-yl)cyclohexanecarboxamide

To a solution of methyl 2-(((1s,4s)-4-(3-methoxy-4-methylphenylcarbamoyl)cyclohexylamino)methyl)benzoate in THF (2 mL) and Water (1 mL) was added LIOH·H₂O (256 mg, 6.09 mmol). The mixture was stirred at 25° C. for 2 hours. The mixture was poured into water (10 mL) and extracted with EtOAc (20 mL). The organic layer was concentrated and purified by prep-HPLC to give (1s,4s)-N-(3-methoxy-4-methylphenyl)-4-(1-oxoisoindolin-2-yl)cyclohexanecarboxamide (20 mg, 53 μmol, 4.3% yield) was obtained as white solid. LCMS ESI m/z 378.9 [M+H]⁺. ¹HNMR (400 MHz, DMSO-d₆) δ 9.71 (s, 1H), 7.66 (d, J=7.5 Hz, 1H), 7.61-7.53 (m, 2H), 7.50-7.43 (m, 1H), 7.34 (d, J=1.4 Hz, 1H), 7.11 (dd, J=8.0, 1.7 Hz, 1H), 7.02 (d, J=8.1 Hz, 1H), 4.45 (s, 2H), 4.21-4.01 (m, 1H), 3.76 (s, 3H), 2.68-2.66 (m, 1H), 2.14-2.07 (m, 5H), 2.04-1.89 (m, 2H), 1.81-1.55 (m, 4H).

### Example 149

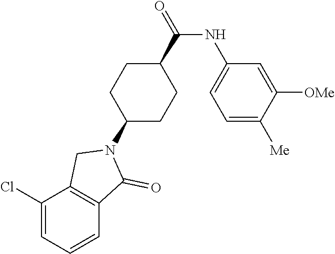 [SMILES: COc1cc(NC(=O)[C@H]2CC[C@@H](N3Cc4c(Cl)cccc4C3=O)CC2)ccc1C]

### (1s,4s)-4-(4-Chloro-1-oxoisoindolin-2-yl)-N-(3-methoxy-4-methylphenyl)cyclohexanecarboxamide

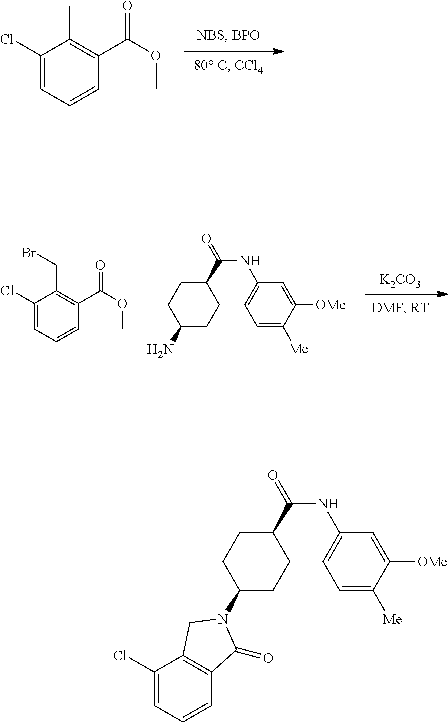

### Step 1

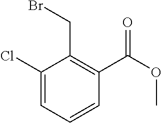

### Methyl 2-(bromomethyl)-3-chlorobenzoate

To a solution of methyl 3-chloro-2-methyl-benzoate (200 mg, 1.08 mmol) was added benzoyl benzenecarboperoxoate (13 mg, 54 μmol) and 1-bromopyrrolidine-2,5-dione (193 mg, 1.08 mmol) and CCl₄ (3 mL). The resulting mixture was stirred at 80° C. for 3 hours. The mixture was diluted with water (20 mL) and extracted with EtOAc (30 mL). The organic layer was dried over anhydrous Na₂SO₄, filtered and concentrated to get crude product, which was purified by silica gel chromatography (PE/EtOAc=40/1 to 20/1) to give methyl 2-(bromomethyl)-3-chlorobenzoate (240 mg, 84% yield) as a yellow oil. ¹H NMR (400 MHz, DMSO-d₆) δ 7.85-7.80 (m, 1H), 7.79-7.72 (m, 1H), 7.53-7.48 (m, 1H), 5.03 (d, J=1.9 Hz, 2H), 3.89 (s, 3H).

### Step 2

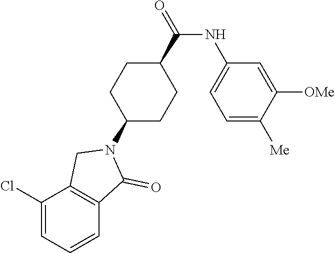 [SMILES: COc1cc(NC(=O)[C@H]2CC[C@@H](N3Cc4c(Cl)cccc4C3=O)CC2)ccc1C]

### (1s,4s)-4-(4-Chloro-1-oxoisoindolin-2-yl)-N-(3-methoxy-4-methylphenyl)cyclohexanecarboxamide

A mixture of methyl 2-(bromomethyl)-3-chlorobenzoate (100 mg, 379 mmol), 4-amino-N-(3-methoxy-4-methyl-phenyl)cyclohexanecarboxamide (100 mg, 379 mmol) and K₂CO₃ (157 mg, 1.14 mmol) in DMF (4 mL) was stirred at 25° C. for 16 hours. To the reaction mixture, EtOAc (20 mL) was added. The solution was washed with water (10 mL*3) and brine. The organic layer was dried, filtered and concentrated to get crude product, which was purified by prep-HPLC to give (1s,4s)-4-(4-chloro-1-oxoisoindolin-2-yl)-N-(3-methoxy-4-methylphenyl)cyclohexanecarboxamide (19 mg, 12% yield) as a white solid. LCMS (ESI) m/z: 412.8 [M+H]⁺. ¹H NMR (400 MHz, DMSO-d₆) δ 9.73 (s, 1H), 7.67 (d, J=7.8 Hz, 2H), 7.54 (t, J=7.7 Hz, 1H), 7.34 (d, J=1.6 Hz, 1H), 7.12 (dd, J=8.0, 1.8 Hz, 1H), 7.02 (d, J=8.1 Hz, 1H), 4.46 (s, 2H), 4.21-4.04 (m, 1H), 3.77 (s, 3H), 2.69-2.66 (m, 1H), 2.16-2.06 (m, 5H), 2.04-2.00 (m, 2H), 1.80-1.57 (m, 4H).

### Example 150

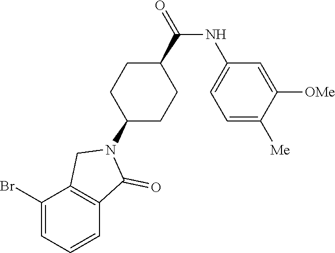 [SMILES: COc1cc(NC(=O)[C@H]2CC[C@@H](N3Cc4c(Br)cccc4C3=O)CC2)ccc1C]

### (1s,4s)-4-(4-Bromo-1-oxoisoindolin-2-yl)-N-(3-methoxy-4-methylphenyl)cyclohexanecarboxamide

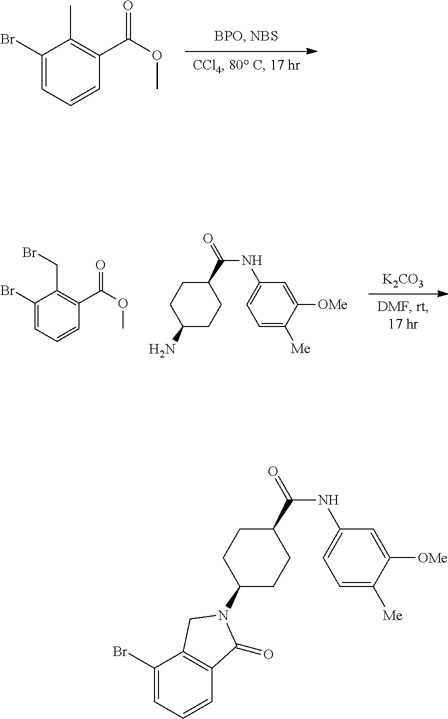

### Step 1

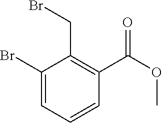

### Methyl 3-bromo-2-(bromomethyl)benzoate

To a solution of methyl 3-bromo-2-methyl-benzoate (5.00 g, 21.8 mmol) in CCl₄ (35 mL) was added BPO (135 mg, 2.18 mmol) and NBS (4.27 g, 24.0 mmol, 2.04 mL) at 80° C. with stirring for 17 hours. The reaction mixture was concentrated and purified by silica gel chromatography (PE/EtOAc=40/1 to 20/1) to give methyl 3-bromo-2-(bromomethyl)benzoate (6.00 g, 19.5 mmol, 89.3% yield) as a yellow oil. ¹HNMR (400 MHz, CDCl₃) δ 7.89 (dd, J=7.8, 1.3 Hz, 1H), 7.76 (dd, J=8.0, 1.3 Hz, 1H), 7.23 (t, J=7.9 Hz, 1H), 5.14 (s, 2H), 3.96 (s, 3H).

### Step 2

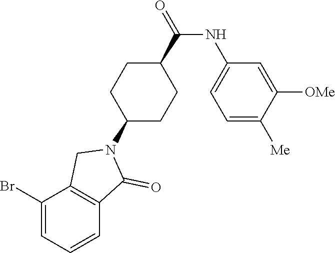 [SMILES: COc1cc(NC(=O)[C@H]2CC[C@@H](N3Cc4c(Br)cccc4C3=O)CC2)ccc1C]

### (1s,4s)-4-(4-Bromo-1-oxoisoindolin-2-yl)-N-(3-methoxy-4-methylphenyl)cyclohexanecarboxamide

To a solution of methyl 3-bromo-2-(bromomethyl)benzoate (300 mg, 974 mmol) in DMF (2 mL) was added 4-amino-N-(3-methoxy-4-methyl-phenyl)cyclohexanecarboxamide (281 mg, 1.07 mmol) and K₂CO₃ (269 mg, 1.95 mmol). The mixture was stirred at 25° C. for 17 hours. To the reaction mixture, EtOAc (40 mL) was added. The solution was washed with water (20 mL*3) and brine. The organic layer was dried, filtered and concentrated to get crude product, which was purified by prep-HPLC to give (1s,4s)-4-(4-bromo-1-oxoisoindolin-2-yl)-N-(3-methoxy-4-methylphenyl)cyclohexanecarboxamide (60 mg, 131 mmol, 13.5% yield) as a white solid. LCMS ESI m/z 457.1 [M+H]⁺. ¹HNMR (400 MHz, DMSO-d₆) δ 9.71 (s, 1H), 7.80 (dd, J=7.9, 0.6 Hz, 1H), 7.70 (d, J=7.0 Hz, 1H), 7.46 (t, J=7.7 Hz, 1H), 7.33 (d, J=1.7 Hz, 1H), 7.12 (dd, J=8.0, 1.8 Hz, 1H), 7.02 (d, J=8.3 Hz, 1H), 4.38 (s, 2H), 4.21-4.03 (m, 1H), 3.76 (s, 3H), 2.68-2.66 (m, 1H), 2.17-2.06 (m, 5H), 1.82-1.55 (m, 4H).

### Example 151

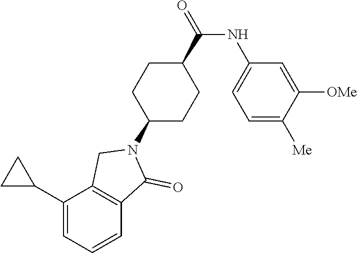 [SMILES: COc1cc(NC(=O)[C@H]2CC[C@@H](N3Cc4c(cccc4C4CC4)C3=O)CC2)ccc1C]

### (1s,4s)-4-(4-Cyclopropyl-1-oxoisoindolin-2-yl)-N-(3-methoxy-4-methylphenyl)cyclohexanecarboxamide

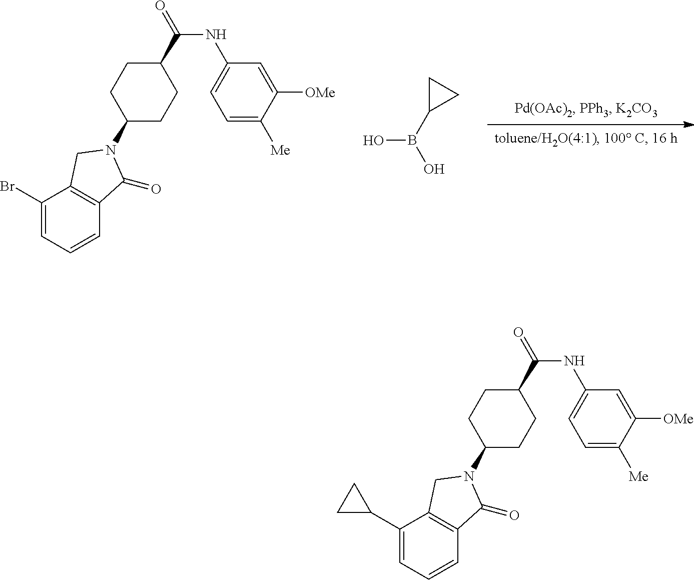

### Step 1

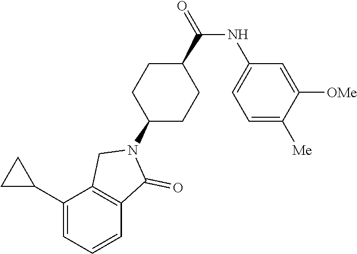 [SMILES: COc1cc(NC(=O)[C@H]2CC[C@@H](N3Cc4c(cccc4C4CC4)C3=O)CC2)ccc1C]

### (1s,4s)-4-(4-Cyclopropyl-1-oxoisoindolin-2-yl)-N-(3-methoxy-4-methylphenyl)cyclohexanecarboxamide

A mixture of (1,4s)-4-(4-bromo-1-oxoisoindolin-2-yl)-N-(3-methoxy-4-methylphenyl)cyclohexanecarboxamide (100 mg, 219 mmol), cyclopropylboronic acid (28.2 mg, 328 mmol), K₂CO₃ (60 mg, 440 mmol), diacetoxypalladium (4.9 mg, 22 mmol), triphenylphosphane (11.5 mg, 43.7 μmol) and water (0.5 mL) in toluene (2 mL) was stirred at 100° C. for 16 hours under N₂. The mixture was filtered and concentrated. The residue was purified by Prep-HPLC to give (1s,4s)-4-(4-cyclopropyl-1-oxoisoindolin-2-yl)-N-(3-methoxy-4-methylphenyl)cyclohexanecarboxamide (5.8 mg, 13.9 mmol, 6.34% yield) as a white solid. LCMS (ESI) m/z: 419.2 [M+H]⁺. ¹H NMR (400 MHz, CDCl₃) δ 7.66 (d, J=8.0 Hz, 1H), 7.37-7.33 (m, 2H), 7.25-7.24 (m, 1H), 7.08-7.03 (m, 2H), 6.83 (dd, J=7.9, 1.8 Hz, 1H), 4.43 (s, 2H), 4.40-4.34 (m, 1H), 3.86 (s, 3H), 2.69-2.67 (m, 1H), 2.26-2.16 (m, 2H), 2.18 (s, 3H), 2.06-2.00 (m, 2H), 1.91-1.80 (m, 5H), 1.03-0.98 (m, 2H), 0.76-0.72 (m, 2H).

### Example 152

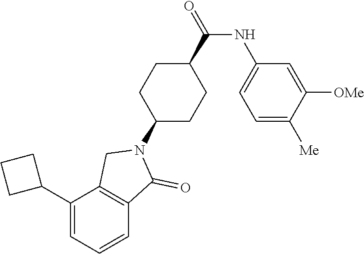 [SMILES: COc1cc(NC(=O)[C@H]2CC[C@@H](N3Cc4c(cccc4C4CCC4)C3=O)CC2)ccc1C]

### (1s,4s)-4-(4-Cyclobutyl-1-oxoisoindolin-2-yl)-N-(3-methoxy-4-methylphenyl)cyclohexanecarboxamide

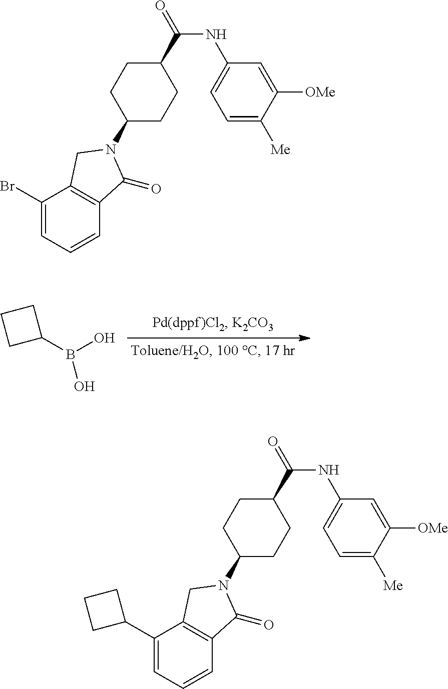 [SMILES: COc1cc(NC(=O)[C@H]2CC[C@@H](N3Cc4c(Br)cccc4C3=O)CC2)ccc1C ; OB(O)C1CCC1 ; COc1cc(NC(=O)[C@H]2CC[C@@H](N3Cc4c(cccc4C4CCC4)C3=O)CC2)ccc1C]

### Step 1

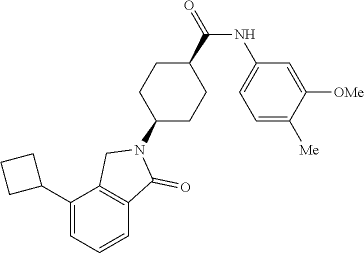 [SMILES: COc1cc(NC(=O)[C@H]2CC[C@@H](N3Cc4c(cccc4C4CCC4)C3=O)CC2)ccc1C]

### (1s,4s)-4-(4-Cyclobutyl-1-oxoisoindolin-2-yl)-N-(3-methoxy-4-methylphenyl)cyclohexanecarboxamide

To a solution of 4-(4-bromo-1-oxo-isoindolin-2-yl)-N-(3-methoxy-4-methyl-phenyl)cyclohexanecarboxamide (250 mg, 547 mmol) and cyclobutylboronic acid (81.9 mg, 820 mmol) in toluene (4 mL) and H₂O (1 mL) was added Pd(dppf)Cl₂ (25 mg, 550 mmol) and K₂CO₃ (500 mg, 547 mmol). The mixture was stirred at 100° C. for 17 hours under N₂. The mixture was poured into water (20 mL) and extracted with EtOAc (20 mL*3). The organic layers were dried and concentrated. The residue was purified by prep-HPLC to (1s,4s)-4-(4-cyclobutyl-1-oxoisoindolin-2-yl)-N-(3-methoxy-4-methylphenyl)cyclohexanecarboxamide (14 mg, 31 mmol, 5.7% yield) as a white solid. LCMS ESI m/z 433.3 [M+H]⁺. ¹H NMR (400 MHz, DMSO-d₆) δ 9.70 (s, 1H), 7.53-7.42 (m, 3H), 7.32 (s, 1H), 7.17-7.09 (m, 1H), 7.02 (d, J=8.1 Hz, 1H), 4.35 (s, 2H), 4.12-4.08 (m, 1H), 3.76 (s, 3H), 3.72-3.61 (m, 1H), 2.68-2.66 (m, 1H), 2.36-2.30 (m, 2H), 2.23-2.06 (m, 7H), 2.05-1.91 (m, 3H), 1.85-1.81 (m, 1H), 1.76-1.69 (m, 2H), 1.63-1.58 (m, 2H).

### Example 153

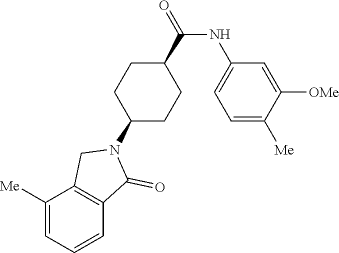 [SMILES: COc1cc(NC(=O)[C@H]2CC[C@@H](N3Cc4c(C)cccc4C3=O)CC2)ccc1C]

### (1s,4s)-N-(3-Methoxy-4-methylphenyl)-4-(4-methyl-1-oxoisoindolin-2-yl)cyclohexanecarboxamide

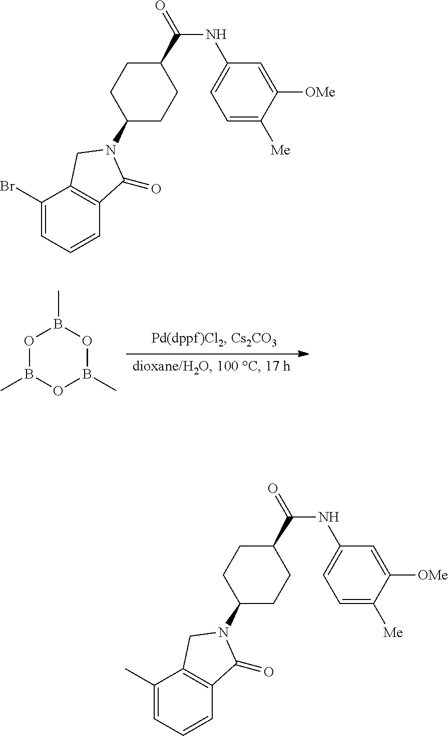

### Step 1

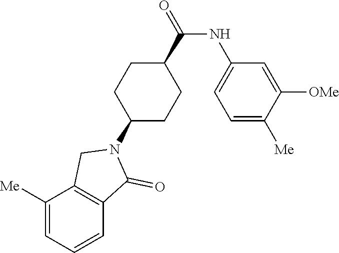 [SMILES: COc1cc(NC(=O)[C@H]2CC[C@@H](N3Cc4c(C)cccc4C3=O)CC2)ccc1C]

### (1s,4s)-N-(3-Methoxy-4-methylphenyl)-4-(4-methyl-1-oxoisoindolin-2-yl)cyclohexanecarboxamide

To a solution of 4-(4-bromo-1-oxo-isoindolin-2-yl)-N-(3-methoxy-4-methyl-phenyl)cyclohexanecarboxamide (100 mg, 219 mmol) and 2,4,6-trimethyl-1,3,5,2,4,6-trioxatriborinane (3.5 M, 125 μL) in H₂O (1 mL) and dioxane (4 mL) was added Cs₂CO₃ (214 mg, 656 mmol) and Pd(dppf)Cl₂ (10 mg, 22 mmol). The mixture was stirred at 100° C. for 17 hours under nitrogen. The mixture was poured into water (20 mL) and extracted with EtOAc (20 mL*3). The organic layers were dried and concentrated. The residue was purified by prep-HPLC to give (1s,4s)-N-(3-methoxy-4-methylphenyl)-4-(4-methyl-1-oxoisoindolin-2-yl)cyclohexanecarboxamide (23 mg, 59 mmol, 27% yield) as a white solid. LCMS ESI m/z 393.2 [M+H]⁺. ¹HNMR (400 MHz, DMSO-d₆) δ 9.71 (s, 1H), 7.54-7.43 (m, 1H), 7.43-7.28 (m, 3H), 7.13 (d, J=7.8 Hz, 1H), 7.02 (d, J=8.0 Hz, 1H), 4.38 (s, 2H), 4.21-4.04 (m, 1H), 3.76 (s, 3H), 2.69-2.67 (m, 1H), 2.33 (s, 3H), 2.19-1.91 (m, 7H), 1.79-1.68 (m, 2H), 1.64-1.59 (m, 2H).

### Example 154

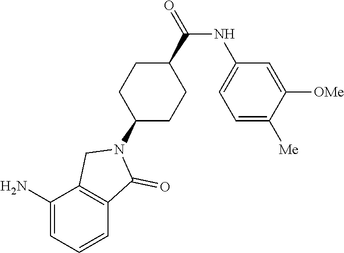 [SMILES: COc1cc(NC(=O)[C@H]2CC[C@@H](N3Cc4c(N)cccc4C3=O)CC2)ccc1C]

### (1s,4s)-4-(4-Amino-1-oxoisoindolin-2-yl)-N-(3-methoxy-4-methylphenyl)cyclohexanecarboxamide

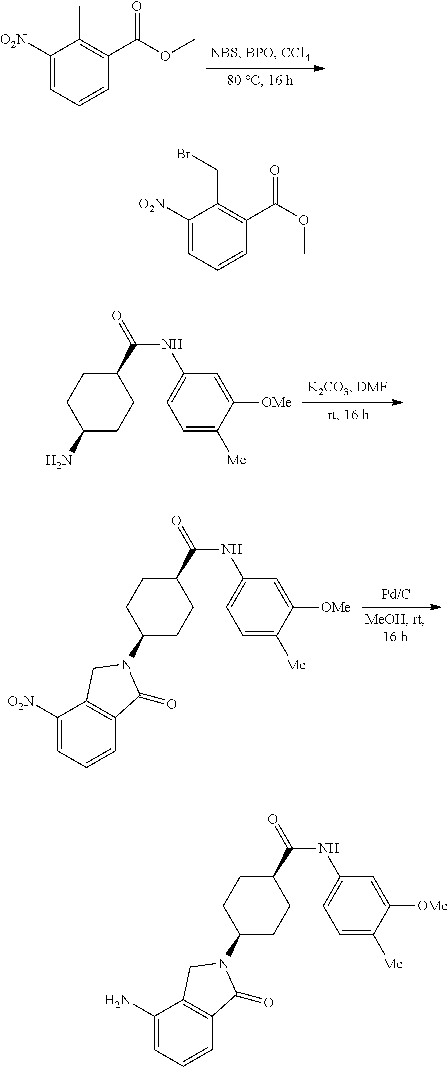 [SMILES: COC(=O)c1cccc([N+](=O)[O-])c1C ; COC(=O)c1cccc([N+](=O)[O-])c1CBr ; COc1cc(NC(=O)[C@H]2CC[C@@H](N)CC2)ccc1C ; COc1cc(NC(=O)[C@H]2CC[C@@H](N3Cc4c(cccc4[N+](=O)[O-])C3=O)CC2)ccc1C ; COc1cc(NC(=O)[C@H]2CC[C@@H](N3Cc4c(N)cccc4C3=O)CC2)ccc1C]

### Step 1 and 2

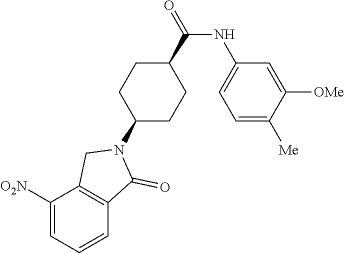 [SMILES: COc1cc(NC(=O)[C@H]2CC[C@@H](N3Cc4c(cccc4[N+](=O)[O-])C3=O)CC2)ccc1C]

### (1s,4s)-N-(3-Methoxy-4-methylphenyl)-4-(4-nitro-1-oxoisoindolin-2-yl)cyclohexanecarboxamide

Following the synthetic procedure for Example 147, but starting with methyl 2-methyl-3-nitrobenzoate gave the title compound as a white solid. LCMS (ESI) m/z: 424.2 [M+H]⁺.

### Step 3

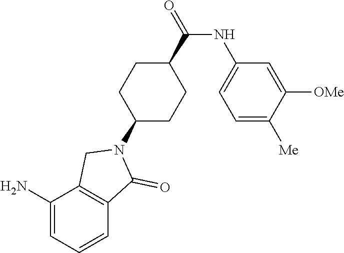 [SMILES: COc1cc(NC(=O)[C@H]2CC[C@@H](N3Cc4c(N)cccc4C3=O)CC2)ccc1C]

### (1s,4s)-4-(4-Amino-1-oxoisoindolin-2-yl)-N-(3-methoxy-4-methylphenyl)cyclohexanecarboxamide

To a solution of (1s,4s)-N-(3-methoxy-4-methylphenyl)-4-(4-nitro-1-oxoisoindolin-2-yl)cyclohexanecarboxamide (200 mg, 472 μmol) in Methanol (10 mL) was added Pd/C (50 mg, wet, 10% Pd). The mixture was stirred at rt for 16 hours under hydrogren atmosphere supplied by a balloon. The mixture was filtered through a celite pad and concentrated. The residue was purified by prep-HPLC to give (1s,4s)-4-(4-amino-1-oxoisoindolin-2-yl)-N-(3-methoxy-4-methylphenyl)cyclohexanecarboxamide (6.8 mg, 17 μmol, 3.67% yield) as a white solid. LCMS ESI m/z: 394.2 [M+H]⁺. ¹H NMR (400 MHz, DMSO-d₆) δ 9.72 (s, 1H), 7.32 (d, J=4.0 Hz, 1H), 7.16-7.10 (m, 2H), 7.02 (d, J=8.0 Hz, 1H), 6.85 (d, J=4.0 Hz, 1H), 6.73 (d, J=4.0 Hz, 1H), 5.54 (s, 2H), 4.18 (s, 2H), 4.12-4.07 (m, 1H), 3.75 (s, 3H), 2.68-2.66 (m, 1H), 2.20-2.08 (m, 5H), 1.98-1.94 (m, 2H), 1.75-1.65 (m, 2H), 1.65-1.58 (m, 2H).

### Example 155

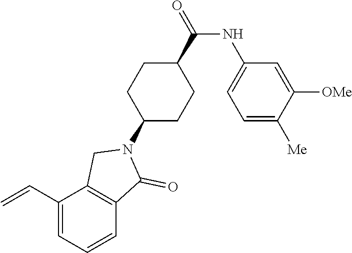 [SMILES: C=Cc1cccc2c1CN([C@H]1CC[C@@H](C(=O)Nc3ccc(C)c(OC)c3)CC1)C2=O]

### (1s,4s)-N-(3-Methoxy-4-methylphenyl)-4-(1-oxo-4-vinylisoindolin-2-yl)cyclohexanecarboxamide

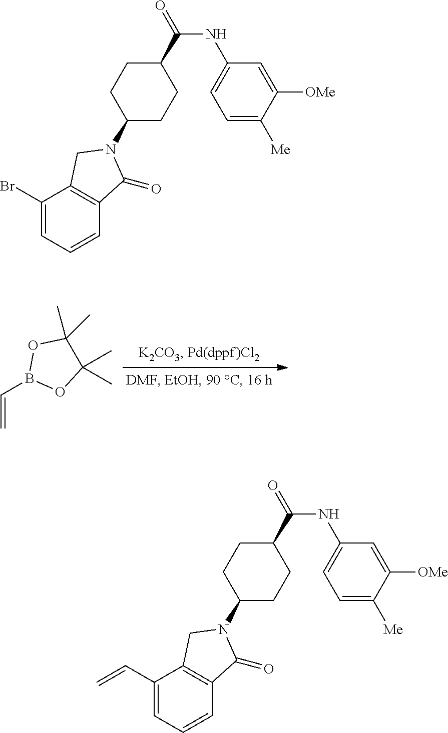 [SMILES: COc1cc(NC(=O)[C@H]2CC[C@@H](N3Cc4c(Br)cccc4C3=O)CC2)ccc1C ; C=CB1OC(C)(C)C(C)(C)O1 ; C=Cc1cccc2c1CN([C@H]1CC[C@@H](C(=O)Nc3ccc(C)c(OC)c3)CC1)C2=O]

### Step 1

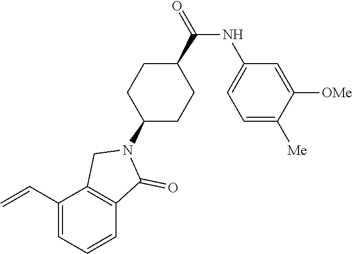 [SMILES: C=Cc1cccc2c1CN([C@H]1CC[C@@H](C(=O)Nc3ccc(C)c(OC)c3)CC1)C2=O]

### (1s,4s)-N-(3-Methoxy-4-methylphenyl)-4-(1-oxo-4-vinylisoindolin-2-yl)cyclohexanecarboxamide

A mixture of 4-(4-bromo-1-oxo-isoindolin-2-yl)-N-(3-methoxy-4-methyl-phenyl)cyclohexanecarboxamide (0.100 g, 219 μmol), 4,4,5,5-tetramethyl-2-vinyl-1,3,2-dioxaborolane (67.4 mg, 437 μmol), potassium carbonate (90.7 mg, 656 μmol, 39.6 μL) and cyclopentyl(diphenyl)phosphane; dichloropalladium; iron (16.0 mg, 21.9 μmol) in DMF (5 mL)/Ethanol (5 mL) was stirred for 16 hours at 90° C. under nitrogen. To the reaction mixture was added EtOAc (50 mL) and washed with H₂O (3×50 mL). The combined organic extracts were dried over Na₂SO₄ and evaporated in vacuo. The residue was purified by Prep-HPLC to get (1s,4s)-N-(3-methoxy-4-methylphenyl)-4-(1-oxo-4-vinylisoindolin-2-yl)cyclohexanecarboxamide (0.070 g, 170 μmol, 79% yield) as a white solid. LCMS ESI m/z: 405.2 [M+H]⁺. ¹H NMR (500 MHz, CDCl₃) δ 7.75 (d, J=7.6 Hz, 1H), 7.61 (d, J=7.6 Hz, 1H), 7.44 (t, J=7.6 Hz, 1H), 7.47-7.27 (m, 2H), 7.06 (d, J=8.0 Hz, 1H), 6.82 (d, J=8.0 Hz, 1H), 6.77-6.67 (m, 1H), 5.76 (d, J=17.6 Hz, 1H), 5.44 (d, J=11.2 Hz, 1H), 4.42 (s, 2H), 4.40-4.30 (m, 1H), 3.86 (s, 3H), 2.69-2.97 (m, 1H), 2.32-2.23 (m, 2H), 2.18 (s, 3H), 2.10-1.97 (m, 2H), 1.94-1.76 (m, 4H).

### Example 156

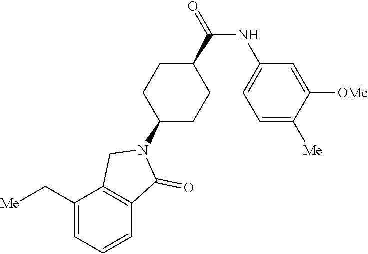 [SMILES: CCc1cccc2c1CN([C@H]1CC[C@@H](C(=O)Nc3ccc(C)c(OC)c3)CC1)C2=O]

### (1s,4s)-4-(4-Ethyl-1-oxoisoindolin-2-yl)-N-(3-methoxy-4-methylphenyl)cyclohexanecarboxamide

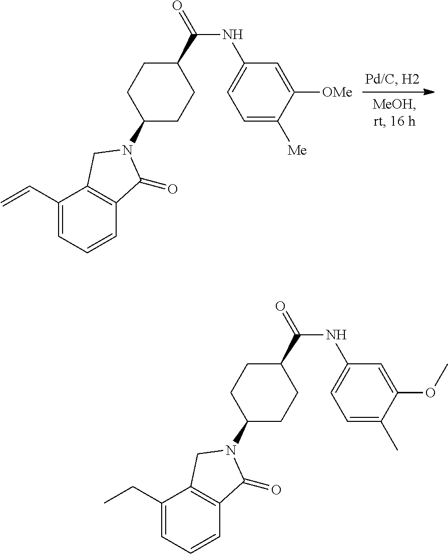 [SMILES: C=Cc1cccc2c1CN([C@H]1CC[C@@H](C(=O)Nc3ccc(C)c(OC)c3)CC1)C2=O ; CCc1cccc2c1CN([C@H]1CC[C@@H](C(=O)Nc3ccc(C)c(OC)c3)CC1)C2=O]

### Step 1

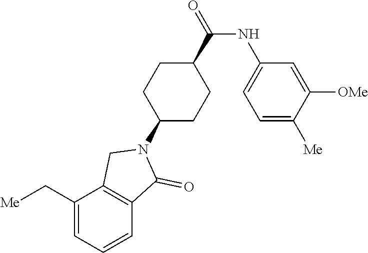 [SMILES: CCc1cccc2c1CN([C@H]1CC[C@@H](C(=O)Nc3ccc(C)c(OC)c3)CC1)C2=O]

### (1s,4s)-4-(4-Ethyl-1-oxoisoindolin-2-yl)-N-(3-methoxy-4-methylphenyl)cyclohexanecarboxamide

Following the synthetic procedure for Example 154, but starting with (1s,4s)-N-(3-methoxy-4-methylphenyl)-4-(1-oxo-4-vinylisoindolin-2-yl)cyclohexanecarboxamide gave the title compound as a white solid. LCMS (ESI) m/z: 407.2 [M+H]⁺. ¹H NMR (400 MHz, CDCl₃) δ 7.68 (d, J=7.2 Hz, 1H), 7.42-7.37 (m, 1H), 7.34 (d, J=7.6 Hz, 2H), 7.32-7.26 (m, 1H), 7.07 (d, J=8.0 Hz, 1H), 6.83 (dd, J=8.0, 2.0 Hz, 1H), 4.43-4.28 (m, 3H), 3.86 (s, 3H), 2.75-2.60 (m, 3H), 2.30-2.22 (m, 2H), 2.18 (s, 3H), 2.10-1.99 (m, 2H), 1.93-1.76 (m, 4H), 1.26 (t, J=7.6 Hz, 3H).

### Example 157

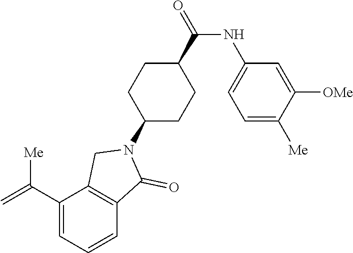 [SMILES: C=C(C)c1cccc2c1CN([C@H]1CC[C@@H](C(=O)Nc3ccc(C)c(OC)c3)CC1)C2=O]

### (1s,4s)-N-(3-Methoxy-4-methylphenyl)-4-(1-oxo-4-(prop-1-en-2-yl)isoindolin-2-yl)cyclohexanecarboxamide

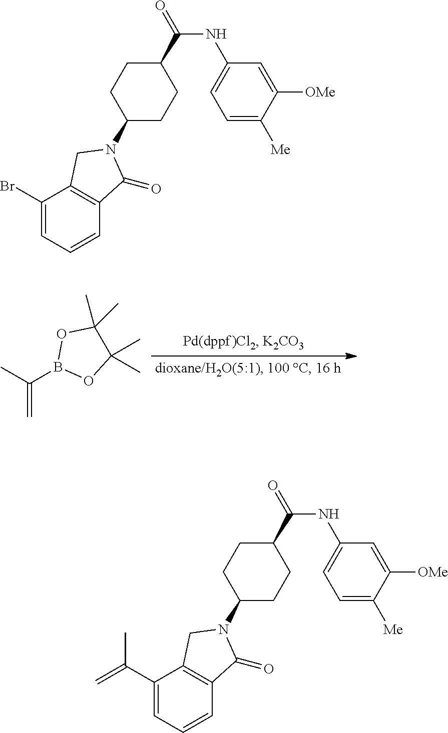

### Step 1

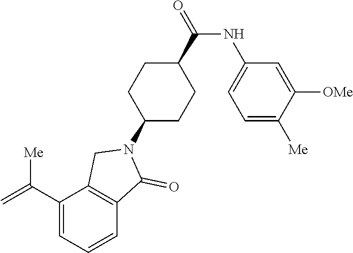 [SMILES: C=C(C)c1cccc2c1CN([C@H]1CC[C@@H](C(=O)Nc3ccc(C)c(OC)c3)CC1)C2=O]

### (1s,4s)-N-(3-Methoxy-4-methylphenyl)-4-(1-oxo-4-(prop-1-en-2-yl)isoindolin-2-yl)cyclohexanecarboxamide

A mixture of (1s,4s)-4-(4-bromo-1-oxoisoindolin-2-yl)-N-(3-methoxy-4-methylphenyl)cyclohexanecarboxamide (100 mg, 219 μmol), 4,4,5,5-tetramethyl-2-(prop-1-en-2-yl)-1,3,2-dioxaborolane (36.7 mg, 219 μmol), K₂CO₃ (90.7 mg, 656 μmol) and Pd(dppf)Cl₂ (24 mg, 33 μmol) in dioxane/H₂O (5/1, 5 mL) was stirred at 100° C. for 16 hours under nitrogen. The mixture was filtered and concentrated. The residue was purified by column chromatography flash, followed by prep-HPLC to give (1s,4s)-N-(3-methoxy-4-methylphenyl)-4-(1-oxo-4-(prop-1-en-2-yl)isoindolin-2-yl)cyclohexanecarboxamide (8.0 mg, 8.0% yield) as a white solid. LCMS (ESI) m/z: 419.2 [M+H]⁺. ¹H NMR (400 MHz, CDCl₃) δ 7.79-7.73 (m, 1H), 7.44 (d, J=4.0 Hz, 2H), 7.34 (s, 1H), 7.07 (d, J=7.9 Hz, 1H), 6.81 (d, J=6.6 Hz, 1H), 5.29 (s, 1H), 5.11 (s, 1H), 4.41 (s, 2H), 4.39-4.29 (m, 1H), 3.86 (s, 3H), 2.72-2.64 (m, 1H), 2.28-2.18 (m, 2H), 2.18 (s, 3H), 2.14 (s, 3H), 2.04-1.87 (m, 2H), 1.91-1.78 (m, 4H).

### Example 158

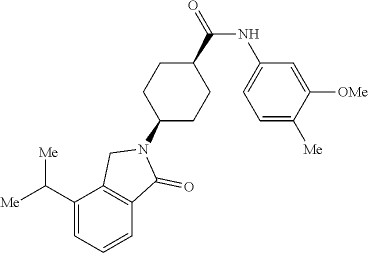 [SMILES: COc1cc(NC(=O)[C@H]2CC[C@@H](N3Cc4c(cccc4C(C)C)C3=O)CC2)ccc1C]

### (1s,4s)-4-(4-Isopropyl-1-oxoisoindolin-2-yl)-N-(3-methoxy-4-methylphenyl)cyclohexanecarboxamide

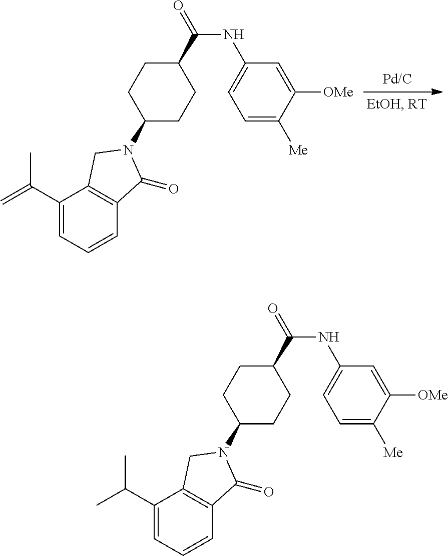 [SMILES: C=C(C)c1cccc2c1CN([C@H]1CC[C@@H](C(=O)Nc3ccc(C)c(OC)c3)CC1)C2=O ; COc1cc(NC(=O)[C@H]2CC[C@@H](N3Cc4c(cccc4C(C)C)C3=O)CC2)ccc1C]

### Step 1

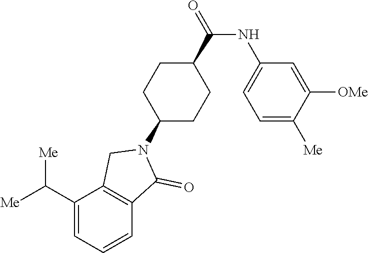 [SMILES: COc1cc(NC(=O)[C@H]2CC[C@@H](N3Cc4c(cccc4C(C)C)C3=O)CC2)ccc1C]

### (1s,4s)-4-(4-Isopropyl-1-oxoisoindolin-2-yl)-N-(3-methoxy-4-methylphenyl)cyclohexanecarboxamide

A mixture of (1s,4s)-N-(3-methoxy-4-methylphenyl)-4-(1-oxo-4-(prop-1-en-2-yl)isoindolin-2-yl)cyclohexanecarboxamide (50.0 mg, 119 μmol) and Pd/C (10 mg, 10% Pd) in EtOH (2 mL) was stirred at room temperature for 16 hours under hydrogen gas atmosphere supplied by balloon. The mixture was filtered through a celite pad and the filtrate was concentrated to get crude product, which was purified by prep-HPLC to give (1s,4s)-4-(4-isopropyl-1-oxoisoindolin-2-yl)-N-(3-methoxy-4-methylphenyl)cyclohexanecarboxamide (2.0 mg, 4.2% yield) as a white solid. LCMS (ESI) m/z: 421.2 [M+H]⁺. ¹H NMR (400 MHz, CDCl₃) δ 7.68 (d, J=5.6 Hz, 1H), 7.41 (s, 2H), 7.34 (s, 1H), 7.07 (d, J=7.4 Hz, 1H), 6.83 (d, J=6.9 Hz, 1H), 4.36-4.33 (m, 3H), 3.86 (s, 3H), 3.02-2.93 (m, 1H), 2.69-2.67 (m, 1H), 2.28-2.64 (m, 2H), 2.18 (s, 3H), 2.06-2.02 (m, 3H), 1.88-1.82 (m, 4H), 1.28 (d, J=6.4 Hz, 6H).

### Example 159 and Example 160

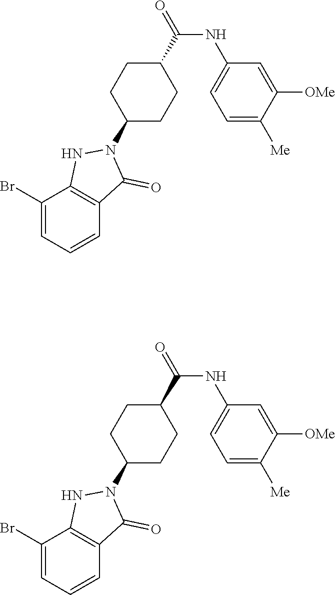 [SMILES: COc1cc(NC(=O)[C@H]2CC[C@H](n3[nH]c4c(Br)cccc4c3=O)CC2)ccc1C ; COc1cc(NC(=O)[C@H]2CC[C@@H](n3[nH]c4c(Br)cccc4c3=O)CC2)ccc1C]

### (1r,4r)-4-(7-Bromo-3-oxo-1H-indazol-2(3H)-yl)-N-(3-methoxy-4-methylphenyl)cyclohexanecarboxamide and (1s,4s)-4-(7-Bromo-3-oxo-1H-indazol-2(3H)-yl)-N-(3-methoxy-4-methylphenyl)cyclohexanecarboxamide

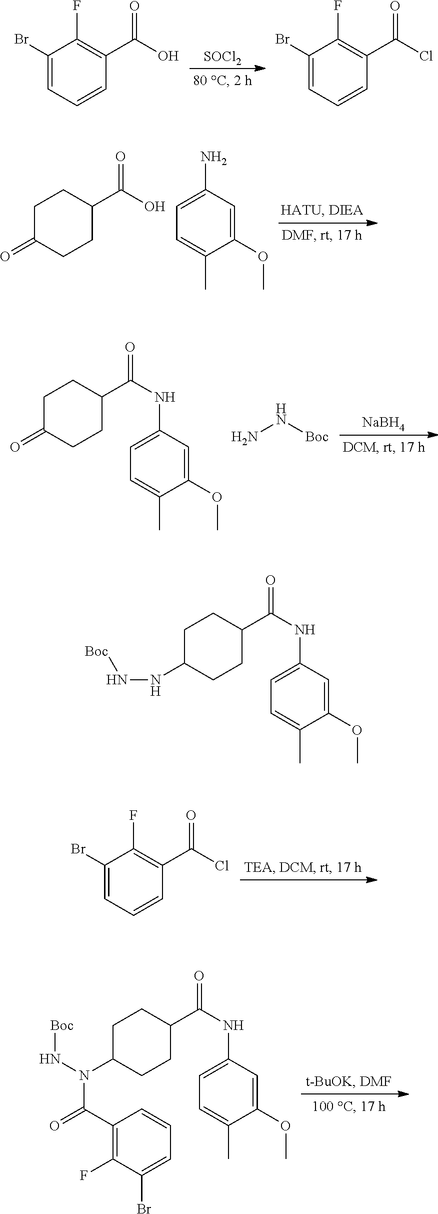

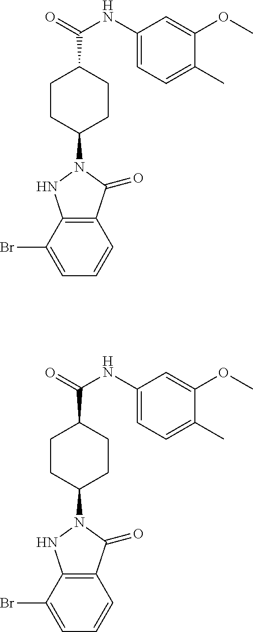 [SMILES: COc1cc(NC(=O)[C@H]2CC[C@H](n3[nH]c4c(Br)cccc4c3=O)CC2)ccc1C ; COc1cc(NC(=O)[C@H]2CC[C@@H](n3[nH]c4c(Br)cccc4c3=O)CC2)ccc1C]

### Intermediate

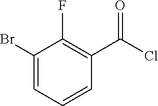 [SMILES: O=C(Cl)c1cccc(Br)c1F]

### 3-Bromo-2-fluorobenzoyl Chloride

A solution of 3-bromo-2-fluoro-benzoic acid (200 mg, 913 μmol) in SOCl₂ (12.5 mL) was stirred at 80° C. for 2 hours. The mixture was concentrated in vacuum to give 3-bromo-2-fluoro-benzoyl chloride (crude) as yellow oil. The crude product was used directly in the next step.

### Step 1

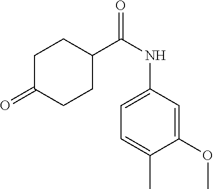 [SMILES: COc1cc(NC(=O)C2CCC(=O)CC2)ccc1C]

### N-(3-Methoxy-4-methylphenyl)-4-oxocyclohexanecarboxamide

To a solution of 4-oxocyclohexanecarboxylic acid (5.00 g, 35.2 mmol) in DMF (50 mL) was added HATU (16.05 g, 42.21 mmol) and DIPEA (13.64 g, 105.5 mmol, 18.38 mL). The mixture was stirred for 1 hour at 25° C., and then 3-methoxy-4-methyl-aniline (5.31 g, 38.7 mmol) was added. The resulting mixture was stirred at 25° C. for another 16 hours. Water (50 mL) was added and the mixture was extracted with EtOAc (50 mL). The organic layer was washed with brine, dried and concentrated. The residue was purified by silica gel chromatography (PE:EtOAc=50:1 to 1:1) to give N-(3-methoxy-4-methyl-phenyl)-4-oxo-cyclohexanecarboxamide (5.00 g, 19.1 mmol, 54.4% yield) as a yellow solid. LCMS (ESI) m/z: 262.0 [M+H]⁺.

### Step 2

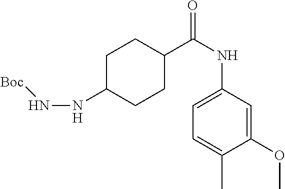 [SMILES: COc1cc(NC(=O)C2CCC(NNC(=O)OC(C)(C)C)CC2)ccc1C]

### tert-Butyl 2-(4-(3-methoxy-4-methylphenylcarbamoyl)cyclohexyl)hydrazinecarboxylate

To a solution of N-(3-methoxy-4-methyl-phenyl)-4-oxo-cyclohexanecarboxamide (200 mg, 0.765 mmol) in DCM (5 mL) was added NH₂NHBoc (152 mg, 1.15 mmol) and NaBH₄ (57.9 mg, 1.53 mmol). The mixture was stirred at 25° C. for 17 hours. The mixture was poured into water (10 mL) and extracted with DCM (10 mL*3). The organic phases were dried and concentrated in vacuo. The residue was purified by silica column (PE:EtOAc=30:1 to 1:1) to give tert-butyl 2-(4-(3-methoxy-4-methylphenylcarbamoyl)cyclohexyl)hydrazinecarboxylate (200 mg, 0.53 mmol, 69% yield) as a yellow solid. LCMS ESI m/z 322.1 [M-55+H]⁺.

### Step 3

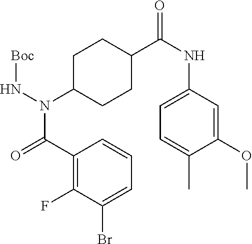 [SMILES: COc1cc(NC(=O)C2CCC(N(NC(=O)OC(C)(C)C)C(=O)c3cccc(Br)c3F)CC2)ccc1C]

### tert-Butyl 2-(3-bromo-2-fluorobenzoyl)-2-(4-(3-methoxy-4-methylphenylcarbamoyl)cyclohexyl)hydrazinecarboxylate

To a solution of tert-butyl 2-(4-(3-methoxy-4-methylphenylcarbamoyl)cyclohexyl)hydrazinecarboxylate (160 mg, 424 μmol) and 3-bromo-2-fluoro-benzoyl chloride (101 mg, 424 μmol) in DCM (3 mL) was added N,N-diethylethanamine (85.8 mg, 848 μmol, 118 μL). The mixture was stirred at 25° C. for 17 hours. The mixture was poured into water (20 mL) and extracted with EtOAc (10 mL*3). The organic phases were dried and concentrated in vacuo. The residue was purified by silica column (PE:EtOAc=30:1 to 1:1) to give tert-butyl N-[(3-bromo-2-fluoro-benzoyl)-[4-[(3-methoxy-4-methyl-phenyl)carbamoyl]cyclohexyl]amino]carbamate (200 mg, 346 μmol, 81.6% yield) as a yellow solid. LCMS ESI m/z 524.1 [M-55+H]⁺.

### Step 4

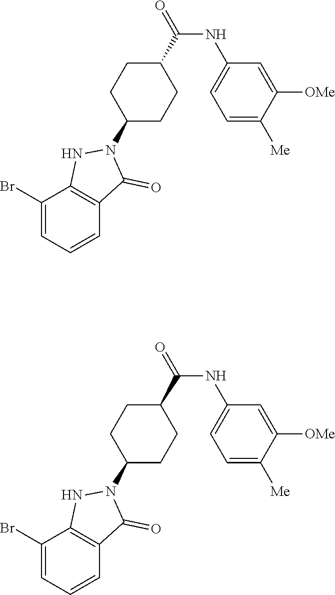 [SMILES: COc1cc(NC(=O)[C@H]2CC[C@H](n3[nH]c4c(Br)cccc4c3=O)CC2)ccc1C ; COc1cc(NC(=O)[C@H]2CC[C@@H](n3[nH]c4c(Br)cccc4c3=O)CC2)ccc1C]

### (1r,4r)-4-(7-Bromo-3-oxo-1H-indazol-2(3H)-yl)-N-(3-methoxy-4-methylphenyl)cyclohexanecarboxamide and (1s,4s)-4-(7-Bromo-3-oxo-1H-indazol-2(3H)-yl)-N-(3-methoxy-4-methylphenyl)cyclohexanecarboxamide

To a solution of tert-butyl 2-(3-bromo-2-fluorobenzoyl)-2-(4-(3-methoxy-4-methylphenylcarbamoyl)cyclohexyl)hydrazinecarboxylate (120 mg, 207 μmol) in DMF (1 mL) was added t-BuOK (69.8 mg, 622 μmol). The mixture was stirred at 100° C. for 17 hours. The mixture was poured into water (10 mL) and extracted with EtOAc (10 mL*3). The organic layers were dried and concentrated. The residue was purified by prep-HPLC to give (1r,4r)-4-(7-bromo-3-oxo-1H-indazol-2(3H)-yl)-N-(3-methoxy-4-methylphenyl)cyclohexanecarboxamide (Example 159) (HPLC Peak 1, 3.4 mg, 3.5% yield) as a white solid and (1s,4s)-4-(7-bromo-3-oxo-1H-indazol-2(3H)-yl)-N-(3-methoxy-4-methylphenyl)cyclohexanecarboxamide (Example 160) (HPLC Peak 2, 5.3 mg, 5.3% yield) as a white solid. Arbitrary isomer assignment of (1r,4r) to Example 159 and (1s,4s) to Example 160.

Example 159: LCMS ESI m/z 460.1 [M+H]⁺. ¹H NMR (400 MHz, DMSO-d₆) δ 9.80 (s, 1H), 7.72-7.63 (m, 2H), 7.35 (s, 1H), 7.08-7.00 (m, 3H), 4.21 (b, 1H), 3.75 (s, 3H), 2.39-2.34 (m, 1H), 2.10-1.83 (m, 9H), 1.65-1.56 (m, 2H).

Example 160: LCMS ESI m/z 460.1 [M+H]⁺. ¹H NMR (400 MHz, DMSO-d₆) δ 10.08 (b, 1H), 9.72 (s, 1H), 7.78-7.57 (m, 2H), 7.39 (d, J=1.6 Hz, 1H), 7.13 (dd, J=8.0, 1.8 Hz, 1H), 7.06-7.00 (m, 2H), 4.29 (b, 1H), 3.76 (s, 3H), 2.68-2.67 (m, 1H), 2.20-2.14 (m, 4H), 2.12-2.04 (m, 3H), 1.81-1.54 (m, 4H).

### Example 161

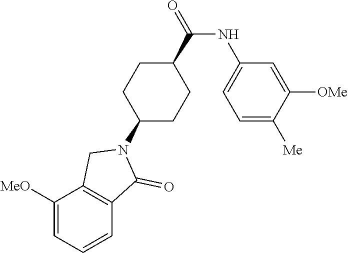 [SMILES: COc1cc(NC(=O)[C@H]2CC[C@@H](N3Cc4c(OC)cccc4C3=O)CC2)ccc1C]

### (1s,4s)-4-(4-Methoxy-1-oxoisoindolin-2-yl)-N-(3-methoxy-4-methylphenyl)cyclohexanecarboxamide

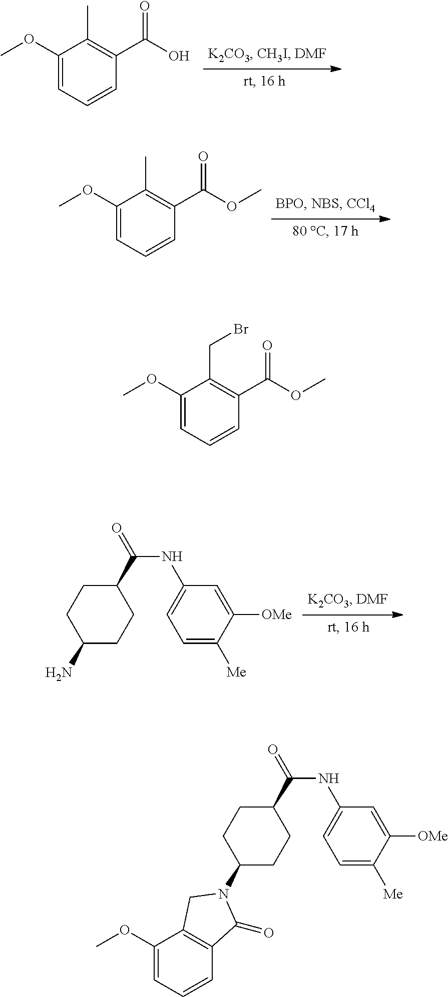

### Step 1

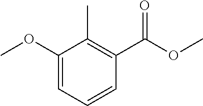

### Methyl 3-methoxy-2-methylbenzoate

A solution of 3-methoxy-2-methyl-benzoic acid (1.00 g, 6.02 mmol), CH₃I (854 mg, 6.02 mmol) and K₂CO₃ (2.50 g, 18.1 mmol) in DMF (10 mL) was stirred at 25° C. for 16 hours. Added EtOAc (50 mL) to the reaction mixture, and the solution was washed with H₂O (3×50 mL). The combined organic extracts were dried over Na₂SO₄ and evaporated in vacuo to get methyl 3-methoxy-2-methylbenzoate (1.1 g, 6.0 mmol) as a white solid.

### Step 2 and 3

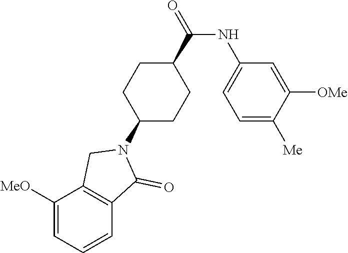 [SMILES: COc1cc(NC(=O)[C@H]2CC[C@@H](N3Cc4c(OC)cccc4C3=O)CC2)ccc1C]

### (1s,4s)-4-(4-Methoxy-1-oxoisoindolin-2-yl)-N-(3-methoxy-4-methylphenyl)cyclohexanecarboxamide

Following the synthetic procedure for Example 147, but starting with methyl 2-methyl-3-nitrobenzoate gave the title compound as a white solid. LCMS (ESI) m/z: 409.2 [M+H]⁺. ¹H NMR (400 MHz, DMSO-d₆) δ 9.70 (s, 1H), 7.47-7.40 (m, 1H), 7.33 (d, J=1.6 Hz, 1H), 7.26 (d, J=8.0 Hz, 1H), 7.19 (d, J=8.0 Hz, 1H), 7.10 (dd, J=1.6, 8.0 Hz, 1H), 7.01 (d, J=8.0 Hz, 1H), 4.34 (s, 2H), 4.15-4.05 (m, 1H), 3.87 (s, 3H), 3.75 (s, 3H), 2.66-2.63 (m, 1H), 2.11-1.85 (m, 7H), 1.81-1.46 (m, 4H).

### Example 162

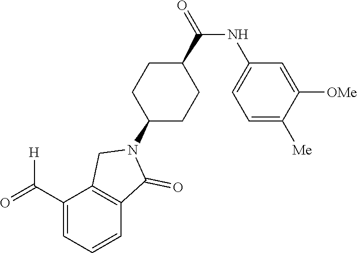 [SMILES: COc1cc(NC(=O)[C@H]2CC[C@@H](N3Cc4c(C=O)cccc4C3=O)CC2)ccc1C]

### (1s,4s)-4-(4-Formyl-1-oxoisoindolin-2-yl)-N-(3-methoxy-4-methylphenyl)cyclohexanecarboxamide

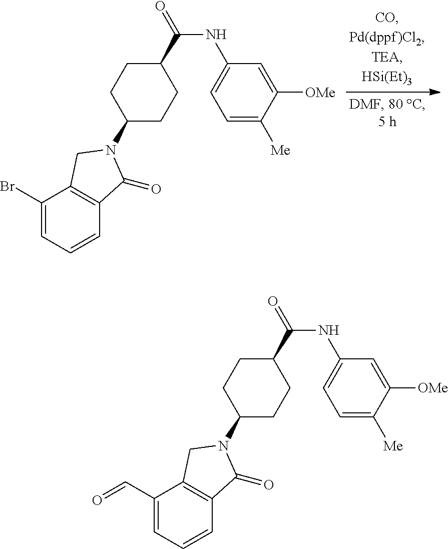 [SMILES: COc1cc(NC(=O)[C@H]2CC[C@@H](N3Cc4c(Br)cccc4C3=O)CC2)ccc1C ; COc1cc(NC(=O)[C@H]2CC[C@@H](N3Cc4c(C=O)cccc4C3=O)CC2)ccc1C]

### Step 1

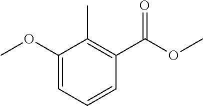

### (1s,4s)-4-(4-Formyl-1-oxoisoindolin-2-yl)-N-(3-methoxy-4-methylphenyl)cyclohexanecarboxamide

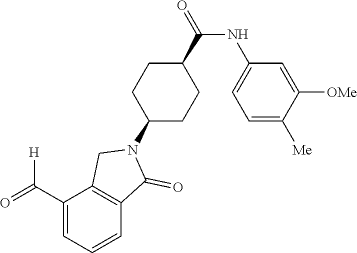 [SMILES: COc1cc(NC(=O)[C@H]2CC[C@@H](N3Cc4c(C=O)cccc4C3=O)CC2)ccc1C]

A solution of 4-(4-bromo-1-oxo-isoindolin-2-yl)-N-(3-methoxy-4-methyl-phenyl)cyclohexanecarboxamide (0.100 g, 219 μmol) and N,N-diethylethanamine (44.3 mg, 437 μmol, 61.0 μL) and cyclopentyl(diphenyl)phosphane; dichloropalladium; iron (16.0 mg, 21.9 μmol) in DMF (5 mL) was stirred for 5 hours at 80° C. under an atmosphere of carbon monoxide supplied by balloon. The mixture was filtered and concentrated. The residue was purified by prep-TLC (PE:EtOAc=1:1) to get (1s,4s)-4-(4-formyl-1-oxoisoindolin-2-yl)-N-(3-methoxy-4-methylphenyl)cyclohexanecarboxamide (3.0 mg, 7.4 μmol, 3.4% yield) as a white solid. LCMS ESI m/z: 407.2 [M+H]⁺. ¹H NMR (400 MHz, CDCl₃) δ 10.16 (s, 1H), 8.10 (d, J=7.6 Hz, 1H), 7.98 (d, J=6.8 Hz, 1H), 7.68 (t, J=7.6 Hz, 1H), 7.47 (s, 1H), 7.21 (b, 1H), 7.06 (d, J=8.0 Hz, 1H), 6.74 (dd, J=8.0, 2.0 Hz, 1H), 4.73 (s, 2H), 4.40-4.25 (m, 1H), 3.89 (s, 2H), 2.68-2.66 (m, 1H), 2.33-2.23 (m, 2H), 2.18 (s, 3H), 2.14-2.04 (m, 2H), 1.92-1.76 (m, 4H).

### Example 163

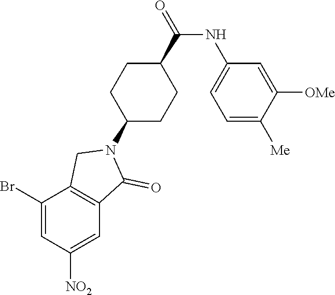 [SMILES: COc1cc(NC(=O)[C@H]2CC[C@@H](N3Cc4c(Br)cc([N+](=O)[O-])cc4C3=O)CC2)ccc1C]

### (1s,4s)-4-(4-Formyl-1-oxoisoindolin-2-yl)-N-(3-methoxy-4-methylphenyl)cyclohexanecarboxamide

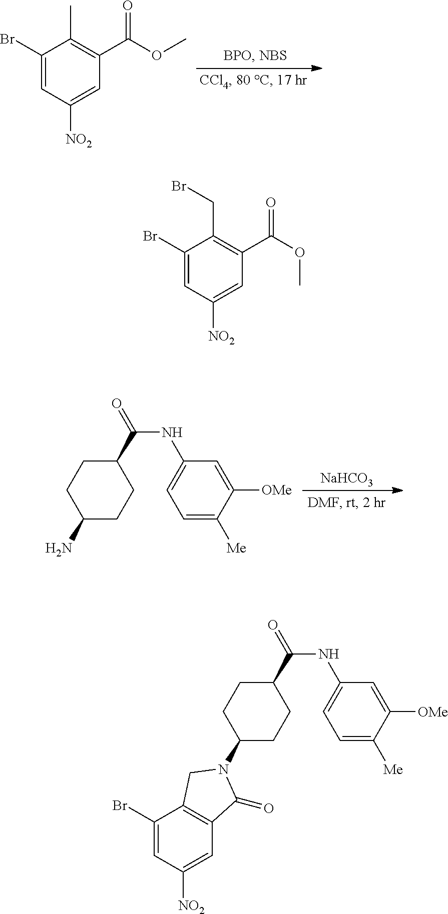

### Step 1

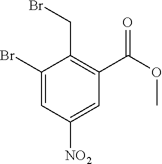 [SMILES: COC(=O)c1cc([N+](=O)[O-])cc(Br)c1CBr]

### Methyl 3-bromo-2-(bromomethyl)-5-nitrobenzoate

To a solution of methyl 3-bromo-2-methyl-5-nitro-benzoate (1.00 g, 3.65 mmol) in CCl₄ (5 mL) was added 1-bromopyrrolidine-2,5-dione (779 mg, 4.38 mmol) and benzoyl benzenecarboperoxoate (88.4 mg, 365 mmol). The mixture was stirred at 80° C. for 17 hours. The mixture was filtered and the filtrate concentrated in vacuo. The residue was purified by silica gel chromatography (PE/EtOAc=40/1 to 20/1) to give methyl 3-bromo-2-(bromomethyl)-5-nitro-benzoate (900 mg, 2.55 mmol, 69.9% yield) as a yellow oil. ¹HNMR (400 MHz, DMSO-d₆) δ 8.69 (d, J=1.9 Hz, 1H), 8.57 (d, J=2.0 Hz, 1H), 5.05 (s, 2H), 3.95 (s, 3H).

### Step 2

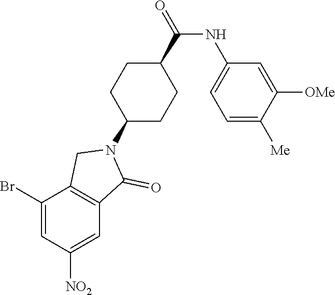 [SMILES: COc1cc(NC(=O)[C@H]2CC[C@@H](N3Cc4c(Br)cc([N+](=O)[O-])cc4C3=O)CC2)ccc1C]

### (1s,4s)-4-(4-Bromo-6-nitro-1-oxoisoindolin-2-yl)-N-(3-methoxy-4-methylphenyl)cyclohexanecarboxamide

To a solution of methyl 3-bromo-2-(bromomethyl)-5-nitro-benzoate (600 mg, 1.70 mmol) and 4-amino-N-(3-methoxy-4-methyl-phenyl)cyclohexanecarboxamide (508 mg, 1.70 mmol, hydrochloride salt) in DMF (1 mL) was added NaHCO₃ (286 mg, 3.40 mmol). The mixture was stirred at 25° C. for 2 hours. The mixture was filtered and concentrated. The residue was purified by prep-HPLC to give (1s,4s)-4-(4-bromo-6-nitro-1-oxoisoindolin-2-yl)-N-(3-methoxy-4-methylphenyl)cyclohexanecarboxamide (65 mg, 130 mmol, 7.6% yield) as a white solid. LCMS ESI m/z 504.1 [M+H]⁺. ¹HNMR (400 MHz, DMSO-d₆) δ 9.72 (s, 1H), 8.63 (d, J=1.9 Hz, 1H), 8.33 (d, J=1.9 Hz, 1H), 7.33 (d, J=1.6 Hz, 1H), 7.12 (dd, J=8.0, 1.7 Hz, 1H), 7.02 (d, J=8.3 Hz, 1H), 4.55 (s, 2H), 4.16-4.11 (m, 1H), 3.76 (s, 3H), 2.68-2.66 (m, 1H), 2.15-1.96 (m, 7H), 1.79-1.63 (m, 4H).

### Example 164

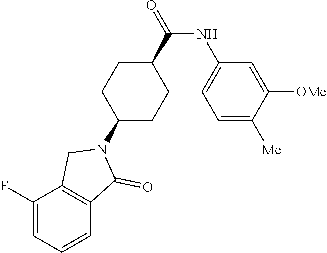 [SMILES: COc1cc(NC(=O)[C@H]2CC[C@@H](N3Cc4c(F)cccc4C3=O)CC2)ccc1C]

### (1s,4s)-4-(4-Fluoro-1-oxoisoindolin-2-yl)-N-(3-methoxy-4-methylphenyl)cyclohexanecarboxamide

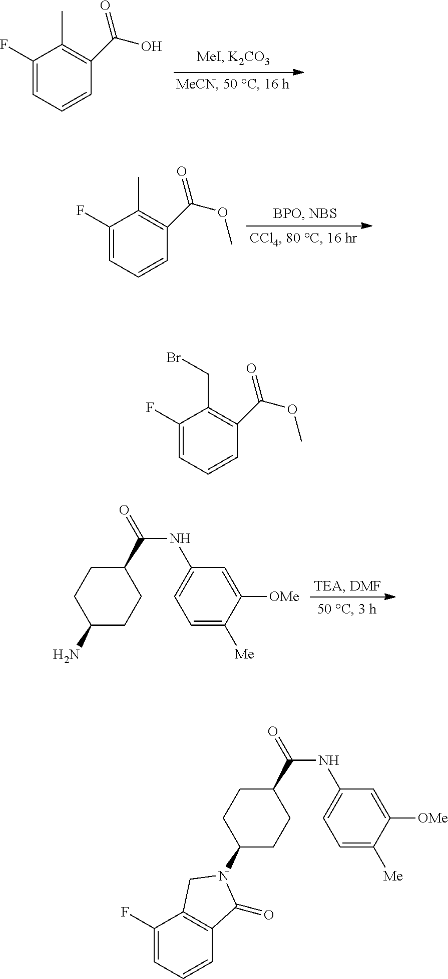

### Step 1

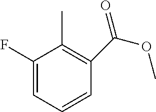

### Methyl 3-fluoro-2-methylbenzoate

To a solution of 3-fluoro-2-methyl-benzoic acid (2.00 g, 13.0 mmol) and potassium carbonate (3.58 g, 26.0 mmol) in MeCN (40 mL) was added iodomethane (2.39 g, 16.9 mmol, 1.05 mL). The mixture was stirred at 50° C. for 16 hours. The mixture was poured into water and extracted with EtOAc. The organic extracts were washed with brine, dried by Na₂SO₄, filtered and the filtrate concentrated to give methyl 3-fluoro-2-methyl-benzoate (1.8 g, 11 mmol, 82% yield) as crude solid. LCMS ESI m/z: 168.1 [M+H]⁺.

### Step 2

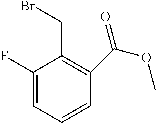

### Methyl 2-(bromomethyl)-3-fluorobenzoate

To a solution of methyl 3-fluoro-2-methyl-benzoate (900 mg, 5.35 mmol) in CCl₄ (20 mL) was added NBS (1.14 g, 6.42 mmol) and benzoyl peroxide (130 mg, 535 mmol). The mixture was stirred at 80° C. for 16 hours. The mixture was filtered and concentrated to give methyl 2-(bromomethyl)-3-fluoro-benzoate (1.0 g, 4.1 mmol, 76% yield) as a yellow oil.

### Step 3

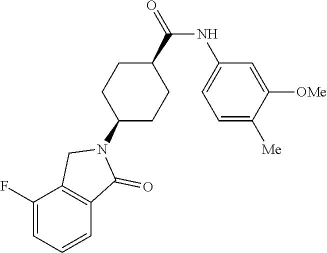 [SMILES: COc1cc(NC(=O)[C@H]2CC[C@@H](N3Cc4c(F)cccc4C3=O)CC2)ccc1C]

### (1s,4s)-4-(4-Fluoro-1-oxoisoindolin-2-yl)-N-(3-methoxy-4-methylphenyl)cyclohexanecarboxamide

To a solution of methyl 2-(bromomethyl)-3-fluoro-benzoate (200 mg, 810 mmol) and 4-amino-N-(3-methoxy-4-methyl-phenyl)cyclohexanecarboxamide (212 mg, 810 mmol) in DMF (4 mL) was added TEA (328 mg, 3.24 mmol). The mixture was stirred at 50° C. for 3 hours. The mixture was poured into water and extracted with EtOAc. The organic extracts were washed with brine, dried by Na₂SO₄, filtered and the filtrate concentrated to give a crude oil. The crude product was purified by prep-HPLC to give (1s,4s)-4-(4-fluoro-1-oxoisoindolin-2-yl)-N-(3-methoxy-4-methylphenyl)cyclohexanecarboxamide (50 mg, 130 mmol, 16% yield) as a white solid. LCMS ESI m/z: 397.2 [M+H]⁺. ¹H NMR (400 MHz, CDCl₃) δ 7.64 (d, J=7.5 Hz, 1H), 7.47-7.40 (m, 1H), 7.38 (d, J=1.7 Hz, 1H), 7.24-7.18 (m, 1H), 7.06 (d, J=8.0 Hz, 1H), 6.79 (dd, J=8.0, 2.0 Hz, 1H), 4.44 (s, 2H), 4.38-4.29 (m, 1H), 3.87 (s, 3H), 2.67-2.65 (m, 1H), 2.28-2.23 (m, 2H), 2.18 (s, 3H), 2.10-1.98 (m, 2H), 1.91-1.76 (m, 4H).

### Example 165

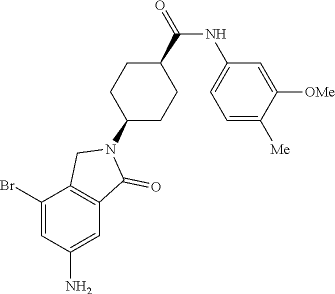 [SMILES: COc1cc(NC(=O)[C@H]2CC[C@@H](N3Cc4c(Br)cc(N)cc4C3=O)CC2)ccc1C]

### (1s,4s)-4-(6-Amino-4-bromo-1-oxoisoindolin-2-yl)-N-(3-methoxy-4-methylphenyl)cyclohexanecarboxamide

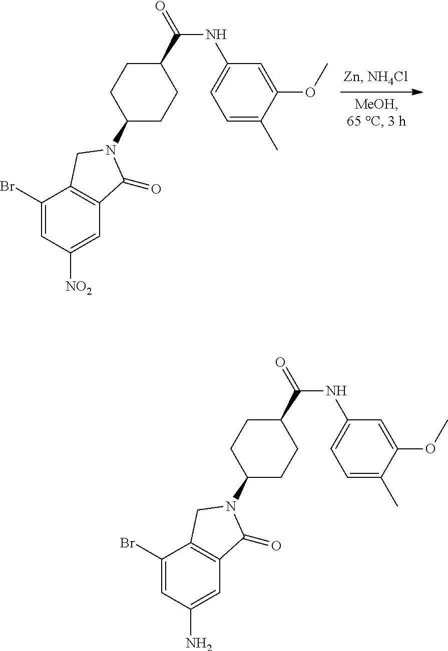 [SMILES: COc1cc(NC(=O)[C@H]2CC[C@@H](N3Cc4c(Br)cc([N+](=O)[O-])cc4C3=O)CC2)ccc1C ; COc1cc(NC(=O)[C@H]2CC[C@@H](N3Cc4c(Br)cc(N)cc4C3=O)CC2)ccc1C]

### Step 1

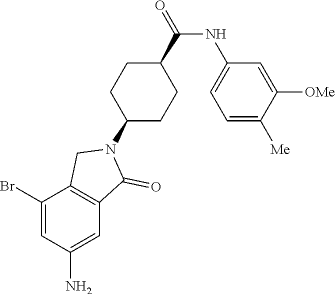 [SMILES: COc1cc(NC(=O)[C@H]2CC[C@@H](N3Cc4c(Br)cc(N)cc4C3=O)CC2)ccc1C]

### (1s,4s)-4-(6-Amino-4-bromo-1-oxoisoindolin-2-yl)-N-(3-methoxy-4-methylphenyl)cyclohexanecarboxamide

To a solution of 4-(4-bromo-6-nitro-1-oxo-isoindolin-2-yl)-N-(3-methoxy-4-methyl-phenyl)cyclohexanecarboxamide (15 mg, 30 mmol) in MeOH (1 mL) was added Zinc (9.7 mg, 150 mmol) and NH₄Cl (8.0 mg, 150 mmol). The mixture was stirred at 65° C. for 3 hours. The mixture was filtered and the filtrate concentrated. The residue was purified by prep-HPLC to give (1s,4s)-4-(6-amino-4-bromo-1-oxoisoindolin-2-yl)-N-(3-methoxy-4-methylphenyl)cyclohexanecarboxamide (3.0 mg, 6.4 mmol, 21% yield) as a white solid. LCMS ESI m/z: 472.1 [M+H]⁺. ¹H NMR (400 MHz, CDCl₃) δ 7.36 (d, J=1.4 Hz, 1H), 7.08-7.02 (m, 2H), 6.96 (d, J=2.0 Hz, 1H), 6.80 (dd, J=7.9, 1.9 Hz, 1H), 4.34-4.25 (m, 1H), 4.19 (s, 2H), 3.86 (s, 5H), 2.67-2.65 (m, 1H), 2.28-2.22 (m, 2H), 2.19 (s, 3H), 2.07-1.98 (m, 2H), 1.89-1.74 (m, 4H).

### Example 166

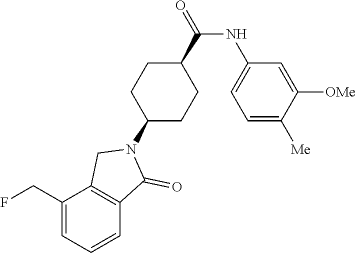 [SMILES: COc1cc(NC(=O)[C@H]2CC[C@@H](N3Cc4c(CF)cccc4C3=O)CC2)ccc1C]

### (1s,4s)-4-(4-(Fluoromethyl)-1-oxoisoindolin-2-yl)-N-(3-methoxy-4-methylphenyl)cyclohexanecarboxamide

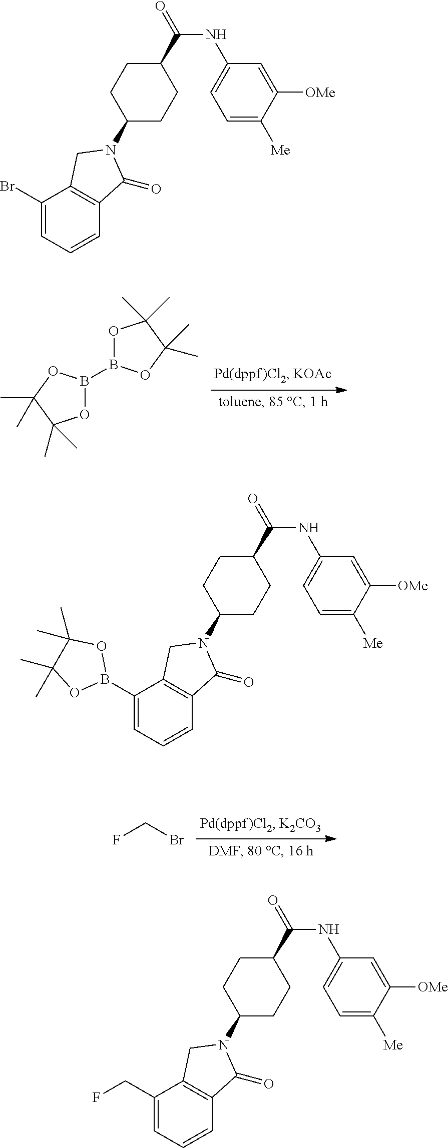

### Step 1

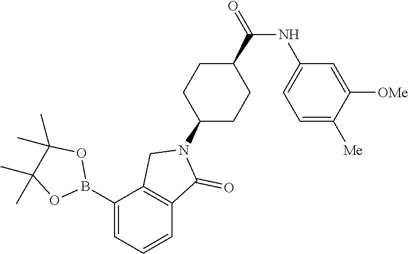 [SMILES: COc1cc(NC(=O)[C@H]2CC[C@@H](N3Cc4c(B5OC(C)(C)C(C)(C)O5)cccc4C3=O)CC2)ccc1C]

### (1s,4s)-N-(3-Methoxy-4-methylphenyl)-4-(1-oxo-4-(4,4,5,5-tetramethyl-1,3,2-dioxaborolan-2-yl)isoindolin-2-yl)cyclohexanecarboxamide

A mixture of (1s,4s)-4-(4-bromo-1-oxoisoindolin-2-yl)-N-(3-methoxy-4-methylphenyl)cyclohexanecarboxamide (125 mg, 273 mmol), 4,4,5,5-tetramethyl-2-(4,4,5,5-tetramethyl-1,3,2-dioxaborolan-2-yl)-1,3,2-dioxaborolane (83.3 mg, 328 mmol), Pd(dppf)Cl₂ (20 mg, 27 mmol) and potassium acetate (54 mg, 550 mmol) in toluene (4 mL) was stirred at 85° C. for 1 hour. The reaction mixture was filtered and the filtrate concentrated. The residue was purified by Prep-HPLC to give (1s,4s)-N-(3-methoxy-4-methylphenyl)-4-(1-oxo-4-(4,4,5,5-tetramethyl-1,3,2-dioxaborolan-2-yl)isoindolin-2-yl)cyclohexanecarboxamide (80 mg, 160 mmol, 58% yield) as yellow oil. LCMS (ESI) m/z: 504.9 [M+H]⁺.

### Step 2

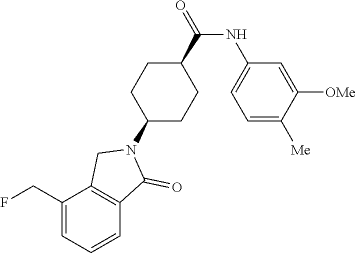 [SMILES: COc1cc(NC(=O)[C@H]2CC[C@@H](N3Cc4c(CF)cccc4C3=O)CC2)ccc1C]

### (1s,4s)-4-(4-(Fluoromethyl)-1-oxoisoindolin-2-yl)-N-(3-methoxy-4-methylphenyl)cyclohexanecarboxamide

A solution of (1s,4s)-N-(3-methoxy-4-methylphenyl)-4-(1-oxo-4-(4,4,5,5-tetramethyl-1,3,2-dioxaborolan-2-yl)isoindolin-2-yl)cyclohexanecarboxamide (0.12 g, 240 mmol), bromo (fluoro) methane (53.7 mg, 476 μmol), potassium carbonate (98.6 mg, 714 μmol) and cyclopentyl(diphenyl)phosphane; dichloropalladium; iron (17.4 mg, 23.8 μmol) in DMF (5.0 mL) was stirred for 16 hours at 80° C. under nitrogen. The reaction mixture was poured into EtOAc (50 mL) and washed with H₂O (3×50 mL). The combined organic extracts were dried over Na₂SO₄ and evaporated in vacuo. The residue was purified by prep-TLC (PE:EtOAc=1:1) and then further purified by prep-HPLC to get (1s,4s)-4-(4-(fluoromethyl)-1-oxoisoindolin-2-yl)-N-(3-methoxy-4-methylphenyl)cyclohexanecarboxamide (0.063 g, 150 μmol, 65% yield) as a white solid. LCMS ESI m/z: 411.2 [M+H]⁺. ¹H NMR (400 MHz, CD₃OD) δ 7.79-7.73 (m, 1H), 7.59-7.50 (m, 2H), 7.29 (d, J=1.6 Hz, 1H), 7.02 (d, J=8.0 Hz, 1H), 6.94 (dd, J=8.0, 2.0 Hz, 1H), 5.62 (d, J=44 Hz, 2H), 4.53 (d, J=32 Hz, 2H), 4.31-4.23 (m, 1H), 3.82 (s, 3H), 2.74-2.72 (m, 1H), 2.24-2.12 (m, 7H), 1.89-1.74 (m, 4H).

### Example 167

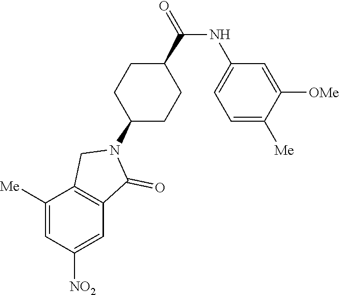 [SMILES: COc1cc(NC(=O)[C@H]2CC[C@@H](N3Cc4c(C)cc([N+](=O)[O-])cc4C3=O)CC2)ccc1C]

### (1s,4s)-N-(3-Methoxy-4-methylphenyl)-4-(4-methyl-6-nitro-1-oxoisoindolin-2-yl)cyclohexanecarboxamide

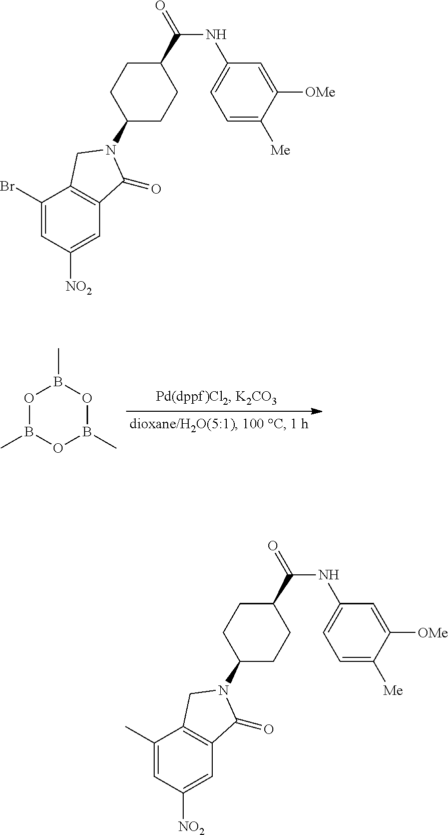

### Step 1

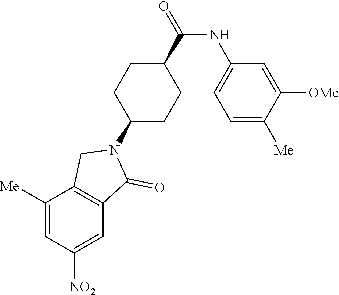 [SMILES: COc1cc(NC(=O)[C@H]2CC[C@@H](N3Cc4c(C)cc([N+](=O)[O-])cc4C3=O)CC2)ccc1C]

### (1s,4s)-N-(3-Methoxy-4-methylphenyl)-4-(4-methyl-6-nitro-1-oxoisoindolin-2-yl)cyclohexanecarboxamide

A mixture of (1s,4s)-4-(4-bromo-6-nitro-1-oxoisoindolin-2-yl)-N-(3-methoxy-4-methylphenyl)cyclohexanecarboxamide (50 mg, 100 mmol), 2,4,6-trimethyl-1,3,5,2,4,6-trioxatriborinane (12.5 mg, 99.5 mmol), K₂CO₃ (41.3 mg, 299 mmol) and Pd(dppf)Cl₂ (7.3 mg, 10 mmol) in dioxane/H₂O (5/1, 2.5 mL) was stirred at 100° C. for 1 hour under nitrogen. The mixture was filtered and the filtrate concentrated. The residue was purified by column chromatography flash, followed by prep-HPLC to give (1s,4s)-N-(3-methoxy-4-methylphenyl)-4-(4-methyl-6-nitro-1-oxoisoindolin-2-yl)cyclohexanecarboxamide (8.5 mg, 19% yield) as a white solid. LCMS (ESI) m/z: 438.2 [M+H]⁺. ¹H NMR (400 MHz, DMSO-d₆) δ 9.72 (s, 1H), 8.29 (s, 1H), 8.18 (s, 1H), 7.34 (s, 1H), 7.14 (d, J=8.0 Hz, 1H), 7.03 (d, J=8.1 Hz, 1H), 4.59 (s, 2H), 4.21-4.07 (m, 1H), 3.77 (s, 3H), 2.71-2.65 (m, 1H), 2.48 (s, 3H), 2.19-1.94 (m, 7H), 1.84-1.53 (m, 4H).

### Example 168

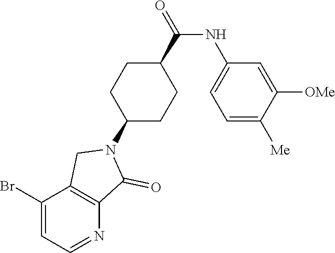 [SMILES: COc1cc(NC(=O)[C@H]2CC[C@@H](N3Cc4c(Br)ccnc4C3=O)CC2)ccc1C]

### (1s,4s)-4-(4-Bromo-7-oxo-5H-pyrrolo[3,4-b]pyridin-6 (7H)-yl)-N-(3-methoxy-4-methylphenyl)cyclohexanecarboxamide

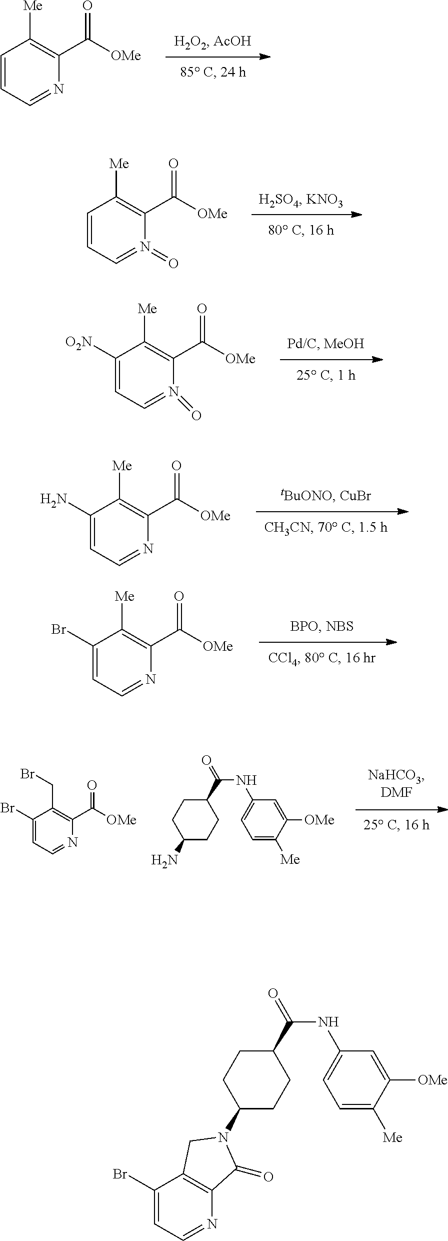

### Step 1

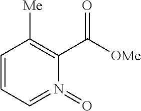 [SMILES: COC(=O)c1c(C)ccc[n+]1[O-]]

### 2-(Methoxycarbonyl)-3-methylpyridine 1-oxide

A solution of aqueous 30% hydrogen peroxide (56 mL, 66 mmol) was added to a solution of methyl 3-methylpicolinate (40.0 g, 264 mmol) in glacial AcOH (150 mL) at room temperature. The mixture was heated in an oil bath for 24 hours, maintaining internal temperature at 85° C. The excess AcOH and water were removed under reduced pressure. The residue was cooled to 0° C. and neutralized with saturated Na₂CO₃ solution, extracted with CH₂Cl₂ and the organic layer was dried (anhydrous Na₂SO₄), concentrated, and purified by chromatography (CH₂Cl₂/MeOH=50/1) to give 2-(methoxycarbonyl)-3-methylpyridine 1-oxide as a faint yellow liquid (29 g, yield 65%). LCMS (ESI) m/z: 168.1 [M+H]⁺.

### Step 2

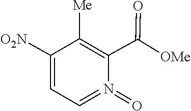 [SMILES: COC(=O)c1c(C)c([N+](=O)[O-])cc[n+]1[O-]]

### 2-(Methoxycarbonyl)-3-methyl-4-nitropyridine 1-oxide

To a solution of 2-(methoxycarbonyl)-3-methylpyridine 1-oxide (25 g, 150 mmol) in H₂SO₄ (200 mL), then added potassium nitrate (121 g, 1.20 mol). The reaction was stirred at 80° C. The reaction mixture was poured into water then added Na₂CO₃ aq. to the solution until pH~10. The separated organic layer was washed with EtOAc (20 mL*3), dried over magnesium sulfate and evaporated to dryness. The crude product was purified by silica gel chromatography eluted with DCM:MeOH=20:1 to give 2-(methoxycarbonyl)-3-methyl-4-nitropyridine 1-oxide (5.3 g, yield 17%). LCMS (ESI) m/z: 213.1 [M+H]⁺.

### Step 3

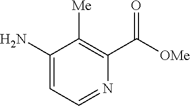 [SMILES: COC(=O)c1nccc(N)c1C]

### Methyl 4-amino-3-methylpicolinate

A mixture of 2-(methoxycarbonyl)-3-methyl-4-nitropyridine 1-oxide (900 mg, 5.21 mmol) and Pd/C (450 mg, 10% Pd) in methanol (60 mL) was stirred for 1 hour at 25° C. TLC (DCM:MeOH=10:1, R_{f}=0.4) showed product was formed. The reaction mixture was filtered through a celite pad and the filtrate was concentrated. The residue was purified by silica gel chromatography (DCM:MeOH=10:1, R_{f}=0.4) to give methyl 4-amino-3-methylpicolinate (0.60 g, 3.6 mmol, 85% yield) as a white solid. ¹H NMR (400 MHz, CDCl₃) δ 8.13 (d, J=5.4 Hz, 1H), 6.60 (d, J=5.4 Hz, 1H), 4.28 (b, 2H), 3.92 (s, 3H), 2.27 (s, 3H).

### Step 4

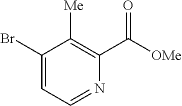 [SMILES: COC(=O)c1nccc(Br)c1C]

### Methyl 4-bromo-3-methylpicolinate

To a solution of tert-butyl nitrite (621 mg, 6.02 mmol, 716 μL) and bromocopper (863 mg, 6.02 mmol) in CH₃CN (30 mL) was stirred for 10 minutes under N₂ at 50° C., methyl 4-amino-3-methyl-pyridine-2-carboxylate (0.5 g, 3.01 mmol) was added. The mixture was stirred for 1 hour at 70° C. The mixture was poured into EtOAc (50 mL) and washed with NH₃·H₂O (50 mL), washed with H₂O (3×50 mL). The combined organic extracts were dried over Na₂SO₄ and evaporated in vacuo. The residue was purified by prep-TLC (PE:EtOAc=3:1) to get methyl 4-bromo-3-methylpicolinate (0.20 g, 870 μmol, 29% yield) as a white solid. LCMS ESI m/z: 309.9 [M+H]⁺.

### Step 5 and 6

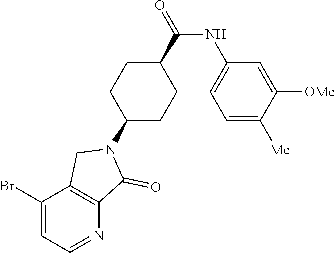 [SMILES: COc1cc(NC(=O)[C@H]2CC[C@@H](N3Cc4c(Br)ccnc4C3=O)CC2)ccc1C]

### (1s,4s)-4-(4-Bromo-7-oxo-5H-pyrrolo[3,4-b]pyridin-6 (7H)-yl)-N-(3-methoxy-4-methylphenyl)cyclohexanecarboxamide

Following the synthetic procedure for Example 163, but starting with methyl 4-bromo-3-methylpicolinate gave the title compound as a white solid. LCMS (ESI) m/z: 458.1 [M+H]⁺. ¹H NMR (400 MHz, CD₃OD) δ 8.56 (d, J=4.8 Hz, 1H), 7.82 (d, J=5.4 Hz, 1H), 7.30 (d, J=2.0 Hz, 1H), 7.04 (d, J=8.0 Hz, 1H), 6.96 (dd, J=8.0, 2.0 Hz, 1H), 4.56 (s, 2H), 4.40-4.30 (m, 1H), 3.81 (s, 3H), 2.68-2.65 (m, 1H), 2.30-2.17 (m, 4H), 2.14 (s, 3H), 1.97-1.69 (m, 4H).

### Example 169

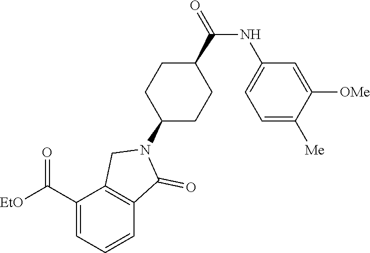 [SMILES: CCOC(=O)c1cccc2c1CN([C@H]1CC[C@@H](C(=O)Nc3ccc(C)c(OC)c3)CC1)C2=O]

### Ethyl 2-((1s,4s)-4-(3-methoxy-4-methylphenylcarbamoyl)cyclohexyl)-1-oxoisoindoline-4-carboxylate

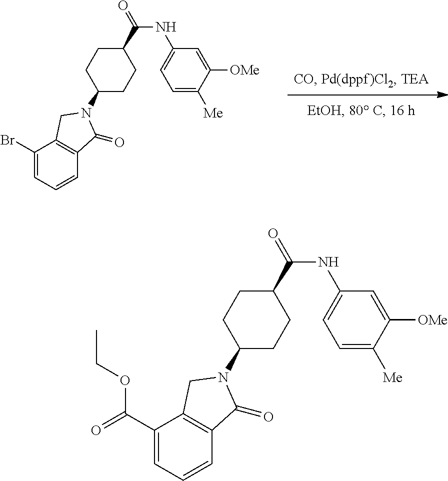 [SMILES: COc1cc(NC(=O)[C@H]2CC[C@@H](N3Cc4c(Br)cccc4C3=O)CC2)ccc1C ; CCOC(=O)c1cccc2c1CN([C@H]1CC[C@@H](C(=O)Nc3ccc(C)c(OC)c3)CC1)C2=O]

### Step 1

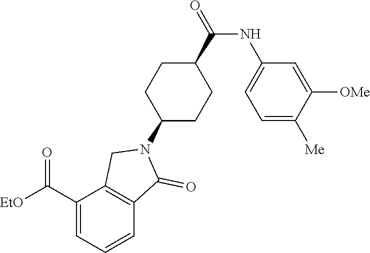 [SMILES: CCOC(=O)c1cccc2c1CN([C@H]1CC[C@@H](C(=O)Nc3ccc(C)c(OC)c3)CC1)C2=O]

### Ethyl 2-((1s,4s)-4-(3-methoxy-4-methylphenylcarbamoyl)cyclohexyl)-1-oxoisoindoline-4-carboxylate

To a solution 4-(4-bromo-1-oxo-isoindolin-2-yl)-N-(3-methoxy-4-methyl-phenyl)cyclohexanecarboxamide (100 mg, 219 mmol) in EtOH (4.0 mL) was added [1,1′-bis(diphenylphosphino)ferrocene]dichloropalladium (II) (48 mg, 66 mmol) and TEA (664 mg, 656 mmol). The mixture was stirred at 80° C. for 16 hours under an atmosphere of carbon monoxide gas supplied by balloon. The mixture was filtered and concentrated to give a crude oil. The crude product was purified by prep-HPLC to give ethyl 2-((1s,4s)-4-(3-methoxy-4-methylphenylcarbamoyl)cyclohexyl)-1-oxoisoindoline-4-carboxylate (25 mg, 55 μmol, 25% yield) as a white solid. LCMS ESI m/z: 450.8 [M+H]⁺. ¹H NMR (400 MHz, CDCl₃) δ 8.18 (dd, J=7.7, 1.1 Hz, 1H), 8.03 (dd, J=7.5, 1.0 Hz, 1H), 7.55 (t, J=7.6 Hz, 1H), 7.46 (d, J=1.4 Hz, 1H), 7.21 (s, 1H), 7.06 (d, J=8.0 Hz, 1H), 6.75 (dd, J=7.9, 2.0 Hz, 1H), 4.71 (s, 2H), 4.41 (q, J=7.1 Hz, 2H), 4.38-4.29 (m, 1H), 3.88 (s, 3H), 2.68-2.66 (m, 1H), 2.29-2.24 (m, 2H), 2.18 (s, 3H), 2.13-2.02 (m, 2H), 1.93-1.79 (m, 4H), 1.42 (t, J=7.1 Hz, 3H).

### Example 170

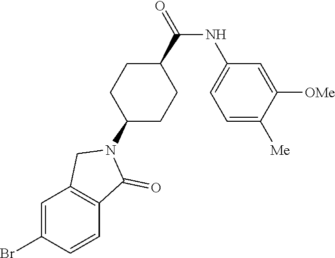 [SMILES: COc1cc(NC(=O)[C@H]2CC[C@@H](N3Cc4cc(Br)ccc4C3=O)CC2)ccc1C]

### (1s,4s)-4-(5-Bromo-1-oxoisoindolin-2-yl)-N-(3-methoxy-4-methylphenyl)cyclohexanecarboxamide

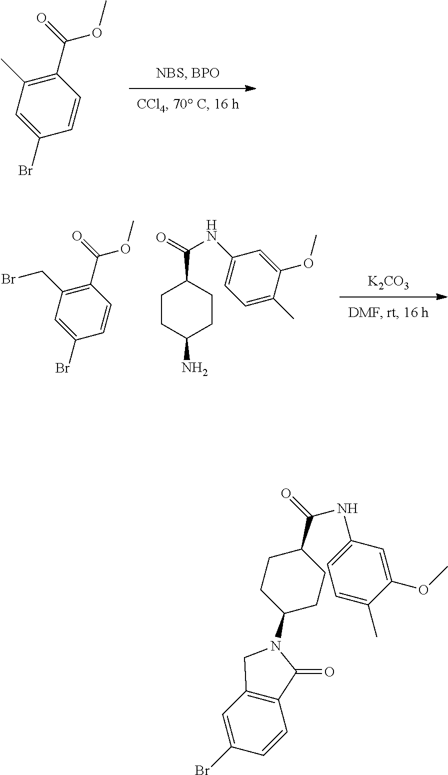

### Step 1

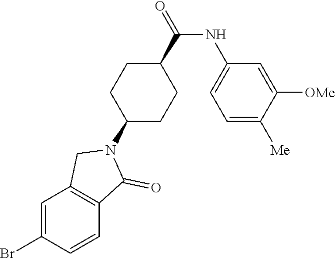 [SMILES: COc1cc(NC(=O)[C@H]2CC[C@@H](N3Cc4cc(Br)ccc4C3=O)CC2)ccc1C]

### (1s,4s)-4-(5-Bromo-1-oxoisoindolin-2-yl)-N-(3-methoxy-4-methylphenyl)cyclohexanecarboxamide

Following the synthetic procedure for Example 147, but starting with methyl 4-bromo-2-methylbenzoate gave the title compound as a white solid. LCMS (ESI) m/z: 457.1 [M+H]⁺. ¹H NMR (400 MHz, CDCl₃) δ 7.73-7.67 (m, 1H), 7.62-7.55 (m, 2H), 7.35 (d, J=1.6 Hz, 1H), 7.21 (s, 1H), 7.06 (d, J=8.0 Hz, 1H), 6.79 (dd, J=7.9, 2.0 Hz, 1H), 4.36 (s, 2H), 4.35-4.32 (M, 1H), 3.86 (s, 3H), 2.66-2.64 (m, 1H), 2.27-2.22 (m, 2H), 2.18 (s, 3H), 2.06-2.00 (m, 2H), 1.89-1.56 (m, 4H).

### Example 171

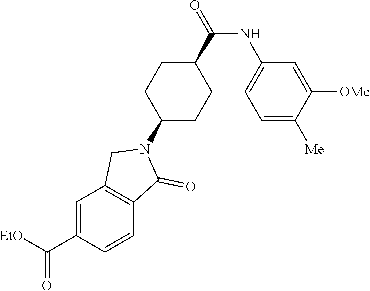 [SMILES: CCOC(=O)c1ccc2c(c1)CN([C@H]1CC[C@@H](C(=O)Nc3ccc(C)c(OC)c3)CC1)C2=O]

### Ethyl 2-((1s,4s)-4-(3-methoxy-4-methylphenylcarbamoyl)cyclohexyl)-1-oxoisoindoline-5-carboxylate

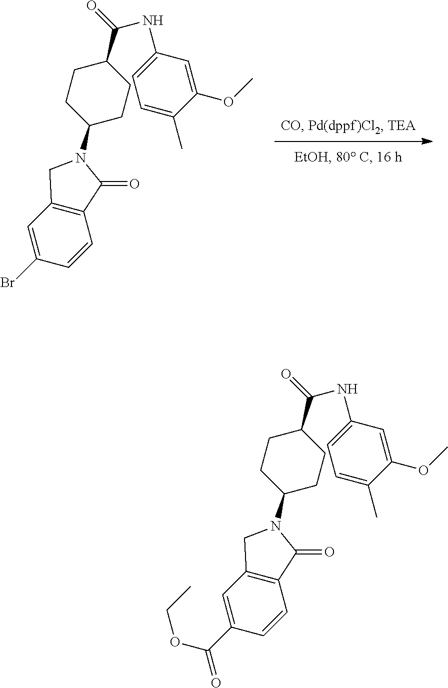 [SMILES: COc1cc(NC(=O)[C@H]2CC[C@@H](N3Cc4cc(Br)ccc4C3=O)CC2)ccc1C ; "CO, Pd(dppf)Cl2, TEA" ; CCOC(=O)c1ccc2c(c1)CN([C@H]1CC[C@@H](C(=O)Nc3ccc(C)c(OC)c3)CC1)C2=O]

### Step 1

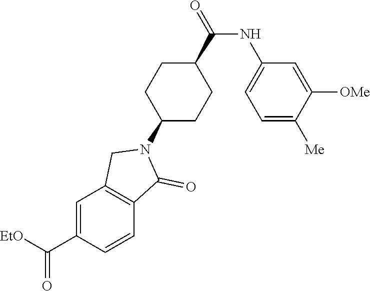 [SMILES: CCOC(=O)c1ccc2c(c1)CN([C@H]1CC[C@@H](C(=O)Nc3ccc(C)c(OC)c3)CC1)C2=O]

### Ethyl 2-((1s,4s)-4-(3-methoxy-4-methylphenylcarbamoyl)cyclohexyl)-1-oxoisoindoline-5-carboxylate

Following the synthetic procedure for Example 169, but starting with (1s,4s)-4-(5-bromo-1-oxoisoindolin-2-yl)-N-(3-methoxy-4-methylphenyl)cyclohexanecarboxamide gave the title compound as a white solid. LCMS (ESI) m/z: 451.2 [M+H]⁺. ¹H NMR (400 MHz, CDCl₃) δ 8.19-8.06 (m, 2H), 7.89 (d, J=7.9 Hz, 1H), 7.36 (s, 1H), 7.21 (b, 1H), 7.06 (d, J=8.0 Hz, 1H), 6.79 (dd, J=7.9, 1.8 Hz, 1H), 4.45-4.33 (m, 5H), 3.86 (s, 3H), 2.68-2.66 (m, 1H), 2.29-2.24 (m, 2H), 2.18 (s, 3H), 2.11-2.01 (m, 2H), 1.88-2.83 (m, 4H), 1.42 (t, J=7.1 Hz, 3H).

### Example 172

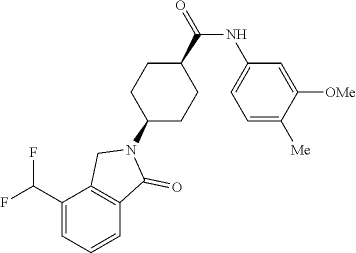 [SMILES: COc1cc(NC(=O)[C@H]2CC[C@@H](N3Cc4c(cccc4C(F)F)C3=O)CC2)ccc1C]

### (1s,4s)-4-(4-(Difluoromethyl)-1-oxoisoindolin-2-yl)-N-(3-methoxy-4-methylphenyl)cyclohexanecarboxamide

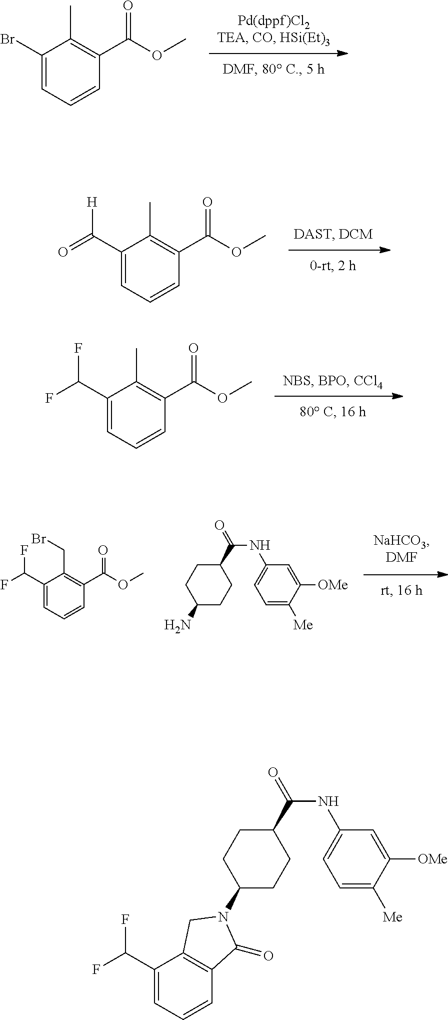

### Step 1

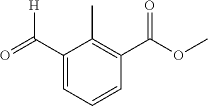

### Methyl 3-formyl-2-methylbenzoate

A solution of methyl 3-bromo-2-methyl-benzoate (0.500 g, 2.18 mmol), N,N-diethylethanamine (442 mg, 4.37 mmol, 608 μL) and cyclopentyl(diphenyl)phosphane; dichloropalladium; iron (160 mg, 218 μmol) in DMF (2 mL) was stirred for 5 h at 80° C. under an atmosphere of carbon monoxide gas supplied by balloon. The mixture was concentrated and the residue purified by flash chromatography on silica gel (0-15% EtOAc in PE) to get methyl 3-formyl-2-methyl-benzoate (0.30 g, 1.7 mmol, 77% yield) as a white solid. ¹H NMR (400 MHz, CDCl₃) δ 10.42 (s, 1H), 8.02-7.93 (m, 2H), 7.42-7.38 (m, 1H), 3.93 (s, 3H), 2.86 (s, 3H)

### Step 2

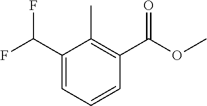

### Methyl 3-(difluoromethyl)-2-methylbenzoate

A solution of methyl 3-formyl-2-methyl-benzoate (0.10 g, 560 μmol) in DCM (2 mL) was added N-ethyl-N-(trifluoro-sulfanyl) ethanamine (452 mg, 2.81 mmol, 371 μL) at 0° C., the mixture was stirred for 2 hours at 25° C. To the reaction mixture was added EtOAc (50 mL) and washed with H₂O (3×50 mL). The combined organic extracts were dried over Na₂SO₄ and evaporated in vacuo. The residue was purified by silica gel chromatography (PE:EtOAc=30:1 to 8:1) to get methyl 3-(difluoromethyl)-2-methylbenzoate (0.080 g, 400 μmol, 71% yield) as yellow oil. ¹H NMR (400 MHz, CDCl₃) δ 7.90 (d, J=7.6 Hz, 1H), 7.68 (d, J=7.6 Hz, 1H), 7.36-7.30 (m, 1H), 6.85 (t, J=55.2 Hz, 1H), 6.71 (s, 4H), 3.90 (s, 3H), 2.60 (s, 3H).

### Step 3 and 4

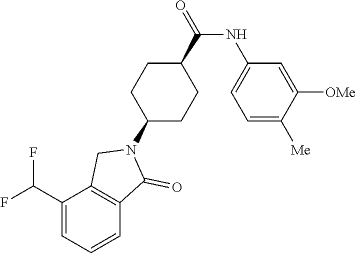 [SMILES: COc1cc(NC(=O)[C@H]2CC[C@@H](N3Cc4c(cccc4C(F)F)C3=O)CC2)ccc1C]

### (1s,4s)-4-(4-(Difluoromethyl)-1-oxoisoindolin-2-yl)-N-(3-methoxy-4-methylphenyl)cyclohexanecarboxamide

Following the synthetic procedure for Example 163, but starting with methyl 3-(difluoromethyl)-2-methylbenzoate gave the title compound as a white solid. LCMS (ESI) m/z: 429.2 [M+H]⁺. ¹H NMR (400 MHz, CDCl₃) δ 7.96 (d, J=7.6 Hz, 1H), 7.62-7.51 (m, 2H), 7.37 (d, J=1.6 Hz, 1H), 7.24 (b, H), 7.06 (d, J=8.0 Hz, 1H), 6.90-6.60 (m, 2H), 4.54 (s, 2H), 4.40-4.32 (m, 1H), 3.87 (s, 3H), 2.68-2.66 (m, 1H), 2.30-2.20 (m, 2H), 2.21 (s, 3H), 2.24-1.99 (m, 2H), 1.99-1.69 (m, 4H).

### Example 173

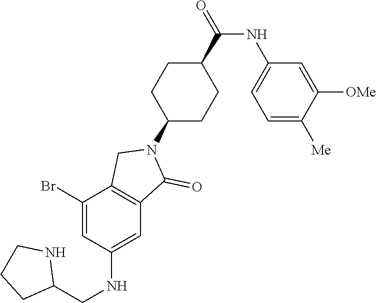 [SMILES: COc1cc(NC(=O)[C@H]2CC[C@@H](N3Cc4c(Br)cc(NCC5CCCN5)cc4C3=O)CC2)ccc1C]

### (1s,4s)-4-(4-Bromo-1-oxo-6-(pyrrolidin-2-ylmethylamino) isoindolin-2-yl)-N-(3-methoxy-4-methylphenyl)cyclohexanecarboxamide

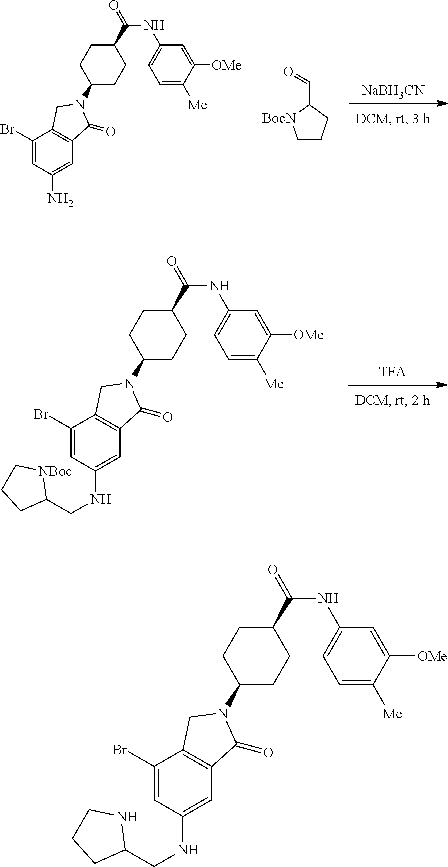 [SMILES: COc1cc(NC(=O)[C@H]2CC[C@@H](N3Cc4c(Br)cc(N)cc4C3=O)CC2)ccc1C ; CC(C)(C)OC(=O)N1CCCC1C=O ; "NaBH3CN" ; "TFA" ; COc1cc(NC(=O)[C@H]2CC[C@@H](N3Cc4c(Br)cc(NCC5CCCN5C(=O)OC(C)(C)C)cc4C3=O)CC2)ccc1C ; COc1cc(NC(=O)[C@H]2CC[C@@H](N3Cc4c(Br)cc(NCC5CCCN5)cc4C3=O)CC2)ccc1C]

### Step 1

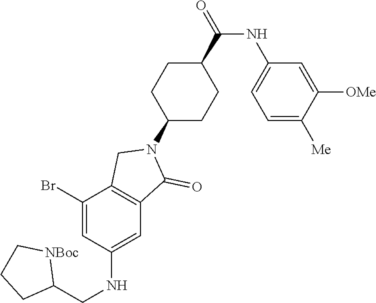 [SMILES: COc1cc(NC(=O)[C@H]2CC[C@@H](N3Cc4c(Br)cc(NCC5CCCN5C(=O)OC(C)(C)C)cc4C3=O)CC2)ccc1C]

### tert-Butyl 2-((7-bromo-2-((1s,4s)-4-(3-methoxy-4-methylphenylcarbamoyl)cyclohexyl)-3-oxoisoindolin-5-ylamino)methyl)pyrrolidine-1-carboxylate

To a solution of 4-(6-amino-4-bromo-1-oxo-isoindolin-2-yl)-N-(3-methoxy-4-methyl-phenyl)cyclohexanecarboxamide (50 mg, 110 mmol) and tert-butyl 2-formylpyrrolidine-1-carboxylate (21 mg, 110 mmol) in DCM (3.0 mL) was added sodium cyanoborohydride (33.3 mg, 529 mmol). The mixture was stirred at 25° C. for 3 hours. The mixture was washed with H₂O, dried by Na₂SO₄, filtered and concentrated to give tert-butyl 2-((7-bromo-2-((1s,4s)-4-(3-methoxy-4-methylphenylcarbamoyl)cyclohexyl)-3-oxoisoindolin-5-ylamino)methyl)pyrrolidine-1-carboxylate (60 mg, 92 mmol, 86% yield) as a white solid. LCMS ESI m/z: 656.3 [M+H]⁺.

### Step 2

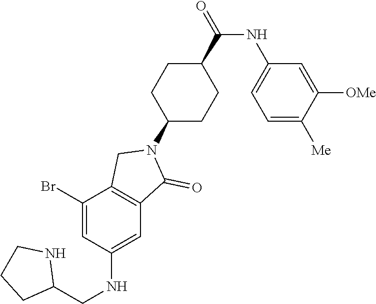 [SMILES: COc1cc(NC(=O)[C@H]2CC[C@@H](N3Cc4c(Br)cc(NCC5CCCN5)cc4C3=O)CC2)ccc1C]

### (1s,4s)-4-(4-Bromo-1-oxo-6-(pyrrolidin-2-ylmethylamino) isoindolin-2-yl)-N-(3-methoxy-4-methylphenyl)cyclohexanecarboxamide

To a solution of tert-butyl 2-((7-bromo-2-((1s,4s)-4-(3-methoxy-4-methylphenylcarbamoyl)cyclohexyl)-3-oxoisoindolin-5-ylamino)methyl)pyrrolidine-1-carboxylate (70 mg, 110 mmol) in DCM (4.0 mL) was added TFA (2.96 g, 26.0 mmol, 2.00 mL). The solution was stirred at 25° C. for 60 mins. The solution was concentrated and purified by prep-HPLC to give (1s,4s)-4-(4-bromo-1-oxo-6-(pyrrolidin-2-ylmethylamino) isoindolin-2-yl)-N-(3-methoxy-4-methylphenyl)cyclohexanecarboxamide (5.0 mg, 9.0 mmol, 8.4% yield) as a white solid. LCMS ESI m/z: 557.2 [M+H]⁺. ¹H NMR (400 MHz, MeOD-d₄) δ 8.53 (b, 1H), 7.29 (d, J=1.8 Hz, 1H), 7.12 (d, J=1.9 Hz, 1H), 7.05-6.98 (m, 2H), 6.95 (dd, J=8.0, 1.9 Hz, 1H), 4.56 (s, 1H), 4.34 (s, 2H), 4.26-4.20 (m, 1H), 3.85-3.77 (m, 4H), 3.48-3.40 (m, 2H), 3.36-3.34 (m, 1H), 3.28-3.26 (m, 1H), 2.74-2.72 (m, 1H), 2.30-2.02 (m, 11H), 1.89-1.73 (m, 5H).

### Example 174

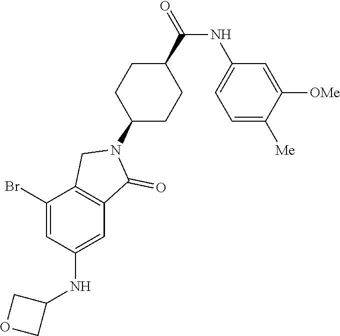 [SMILES: COc1cc(NC(=O)[C@H]2CC[C@@H](N3Cc4c(Br)cc(NC5COC5)cc4C3=O)CC2)ccc1C]

### (1s,4s)-4-(4-Bromo-6-(oxetan-3-ylamino)-1-oxoisoindolin-2-yl)-N-(3-methoxy-4-methylphenyl)cyclohexanecarboxamide

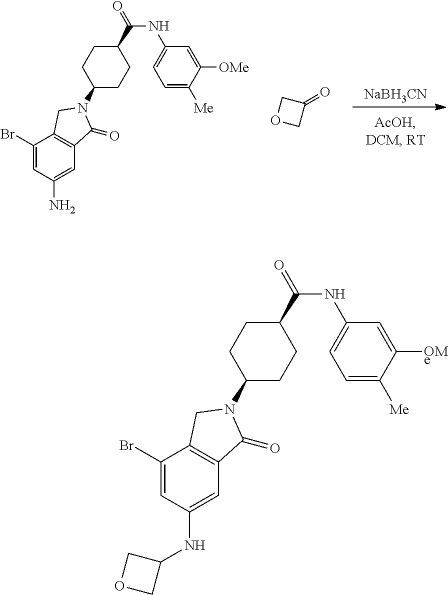 [SMILES: COc1cc(NC(=O)[C@H]2CC[C@@H](N3Cc4c(Br)cc(N)cc4C3=O)CC2)ccc1C ; O=C1COC1 ; "NaBH3CN" ; COc1cc(NC(=O)[C@H]2CC[C@@H](N3Cc4c(Br)cc(NC5COC5)cc4C3=O)CC2)ccc1C]

### Step 1

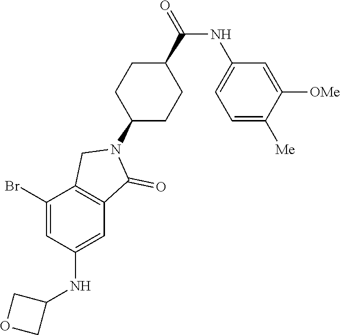 [SMILES: COc1cc(NC(=O)[C@H]2CC[C@@H](N3Cc4c(Br)cc(NC5COC5)cc4C3=O)CC2)ccc1C]

### (1s,4s)-4-(4-Bromo-6-(oxetan-3-ylamino)-1-oxoisoindolin-2-yl)-N-(3-methoxy-4-methylphenyl)cyclohexanecarboxamide

A solution of (1s,4s)-4-(6-amino-4-bromo-1-oxoisoindolin-2-yl)-N-(3-methoxy-4-methylphenyl)cyclohexanecarboxamide (60.0 mg, 127 μmol), oxetan-3-one (91.5 mg, 1.27 mmol) and acetic acid (15 mg, 250 μmol, 15 μL) in DCM (5 mL) was stirred at room temperature for 16 hours. Sodium cyanoborohydride (79.8 mg, 1.27 mmol) was added to the mixture. The resulting mixture was stirred for another 16 hours. The reaction mixture was diluted with water (20 mL) and extracted with EtOAc (20 mL*3). The organic layers were dried over anhydrous Na₂SO₄, filtered and concentrated to get crude product, which was purified by prep-HPLC to give (1s,4s)-4-(4-bromo-6-(oxetan-3-ylamino)-1-oxoisoindolin-2-yl)-N-(3-methoxy-4-methylphenyl)cyclohexanecarboxamide (7.4 mg, 11% yield) as a white solid. LCMS (ESI) m/z: 528.2 [M+H]⁺. ¹H NMR (400 MHz, DMSO-d₆) δ 9.70 (s, 1H), 7.32 (d, J=1.7 Hz, 1H), 7.11 (dd, J=8.0, 1.9 Hz, 1H), 7.02 (d, J=8.5 Hz, 1H), 6.94-6.86 (m, 2H), 6.68 (d, J=1.9 Hz, 1H), 4.87 (t, J=6.5 Hz, 2H), 4.67-4.54 (m, 1H), 4.38 (t, J=6.0 Hz, 2H), 4.19 (s, 2H), 4.12-4.02 (m, 1H), 3.76 (s, 3H), 2.70-2.61 (m, 1H), 2.16-1.87 (m, 7H), 1.78-1.54 (m, 4H).

### Example 175

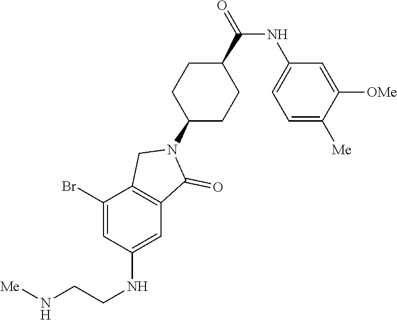 [SMILES: CNCCNc1cc(Br)c2c(c1)C(=O)N([C@H]1CC[C@@H](C(=O)Nc3ccc(C)c(OC)c3)CC1)C2]

### (1s,4s)-4-(4-Bromo-6-(2-(methylamino)ethylamino)-1-oxoisoindolin-2-yl)-N-(3-methoxy-4-methylphenyl)cyclohexanecarboxamide

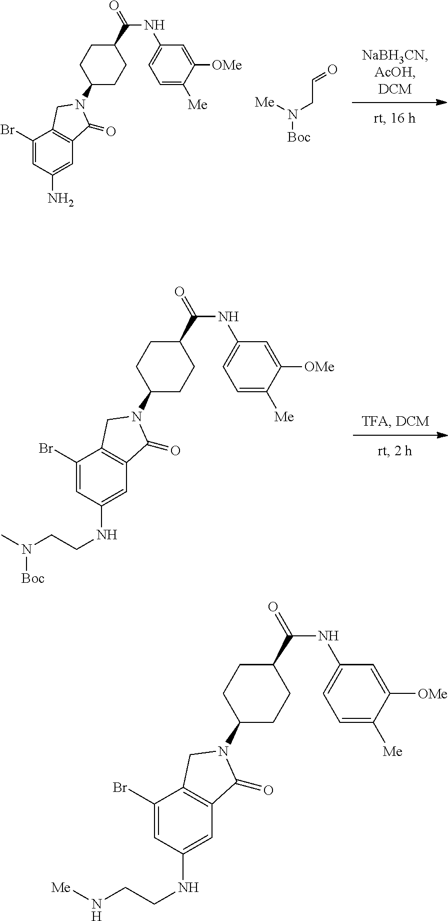

### Step 1

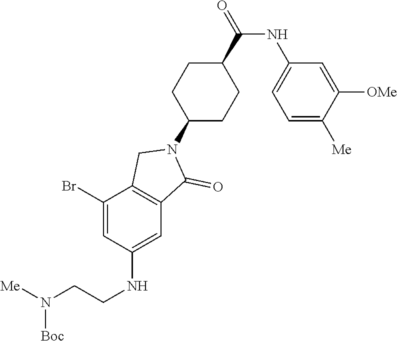 [SMILES: COc1cc(NC(=O)[C@H]2CC[C@@H](N3Cc4c(Br)cc(NCCN(C)C(=O)OC(C)(C)C)cc4C3=O)CC2)ccc1C]

### tert-Butyl 2-(7-bromo-2-((1s,4s)-4-(3-methoxy-4-methylphenylcarbamoyl)cyclohexyl)-3-oxoisoindolin-5-ylamino)ethyl(methyl)carbamate

A solution of 4-(6-amino-4-bromo-1-oxo-isoindolin-2-yl)-N-(3-methoxy-4-methyl-phenyl)cyclohexanecarboxamide (0.050 g, 110 μmol), tert-butyl N-methyl-N-(2-oxoethyl)carbamate (36.7 mg, 212 μmol), acetic acid (13 mg, 210 μmol, 12 μL) and sodium cyanoborohydride (13 mg, 210 μmol) in DCM (2 mL) was stirred for 16 hours at 25° C. To the reaction mixture was added EtOAc (50 mL) and washed with H₂O (3×50 mL). The combined organic extracts were dried over Na₂SO₄ and evaporated in vacuo to get tert-butyl 2-(7-bromo-2-((1s,4s)-4-(3-methoxy-4-methylphenylcarbamoyl)cyclohexyl)-3-oxoisoindolin-5-ylamino)ethyl(methyl)carbamate (0.060 g, 95 μmol, 90% yield) as yellow oil. LCMS ESI m/z: 629.2 [M+H]⁺.

### Step 2

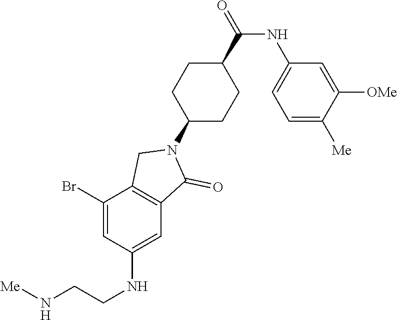 [SMILES: CNCCNc1cc(Br)c2c(c1)C(=O)N([C@H]1CC[C@@H](C(=O)Nc3ccc(C)c(OC)c3)CC1)C2]

### (1s,4s)-4-(4-Bromo-6-(2-(methylamino)ethylamino)-1-oxoisoindolin-2-yl)-N-(3-methoxy-4-methylphenyl)cyclohexanecarboxamide

A solution of tert-butyl 2-(7-bromo-2-((1s,4s)-4-(3-methoxy-4-methylphenylcarbamoyl)cyclohexyl)-3-oxoisoindolin-5-ylamino)ethyl(methyl)carbamate (0.060 g, 95 μmol) in TFA/DCM (1/4, 5 mL) was stirred for 2 hours at 25° C. The solution was concentrated and purified by prep-HPLC to get (1s,4s)-4-(4-bromo-6-(2-(methylamino)ethylamino)-1-oxoisoindolin-2-yl)-N-(3-methoxy-4-methylphenyl)cyclohexanecarboxamide (0.038 g, 72 μmol, 75% yield) as a white solid. LCMS ESI m/z: 529.2 [M+H]⁺. ¹H NMR (400 MHz, CD₃OD) δ 7.28 (s, 1H), 7.15-6.90 (m, 4H), 4.35 (s, 2H), 4.28-4.15 (m, 1H), 3.83 (s, 3H), 3.49-3.45 (m, 2H), 3.22-3.18 (m, 2H), 2.73-2.68 (m, 4H), 2.30-2.10 (m, 7H), 1.90-1.70 (m, 4H).

### Example 176

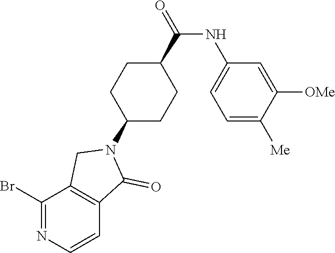 [SMILES: COc1cc(NC(=O)[C@H]2CC[C@@H](N3Cc4c(ccnc4Br)C3=O)CC2)ccc1C]

### (1s,4s)-4-(4-Bromo-1-oxo-1H-pyrrolo[3,4-c]pyridin-2(3H)-yl)-N-(3-methoxy-4-methylphenyl)cyclohexanecarboxamide

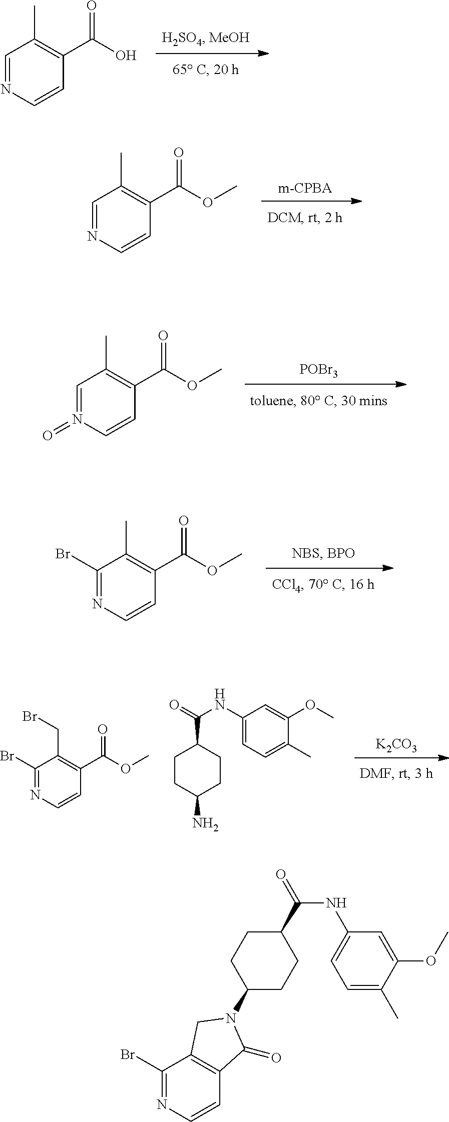

### Step 1

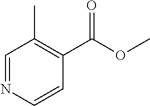

### Methyl 3-methylisonicotinate

To a solution of 3-methylpyridine-4-carboxylic acid (2.00 g, 14.6 mmol) in MeOH (30 mL) was added H₂SO₄ (15 mmol, 5.0 mL). The mixture was stirred at 65° C. for 20 hours. The mixture was concentrated and poured into water. The solution was basified with NaHCO₃ to PH~8 to 9 and extracted with DCM. The organic extracts were washed with brine, dried by Na₂SO₄, filtered and concentrated to give methyl 3-methylpyridine-4-carboxylate (1.7 g, 11 mmol, 77% yield) as a white solid. LCMS ESI m/z: 152.2 [M+H]⁺.

### Step 2

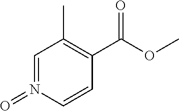

### 4-(Methoxycarbonyl)-3-methylpyridine 1-oxide

A solution of methyl 3-methylpyridine-4-carboxylate (1.7 g, 11 mmol) and m-CPBA (3.88 g, 22.5 mmol) in DCM (20 mL) was stirred at 25° C. for 2 hours. The reaction was quenched by aqueous sodium thiosulfate. The solution was adjusted to PH~7 to 8 with NaHCO₃. The solution was extracted with DCM. The organic extracts were washed with brine, dried by Na₂SO₄, filtered and concentrated to give 4-(methoxycarbonyl)-3-methylpyridine 1-oxide (1.7 g, 10 mmol, 90% yield) as a white solid. LCMS ESI m/z: 168.2 [M+H]⁺.

### Step 3

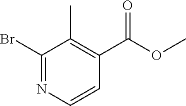

### Methyl 2-bromo-3-methylisonicotinate

To a solution of methyl 3-methyl-1-oxido-pyridin-1-ium-4-carboxylate (1.00 g, 5.98 mmol) in toluene (30 mL) was added phosphorylbromide (3.43 g, 12.0 mmol, 1.22 mL). The mixture was stirred at 80° C. for 30 minutes. The mixture was concentrated to give a crude oil. The crude product was purified by silica gel chromatography to give methyl 2-bromo-3-methyl-pyridine-4-carboxylate (140 mg, 609 μmol, 10.2% yield) as a yellow solid. LCMS ESI m/z: 232.1 [M+H]⁺.

### Step 4 and 5

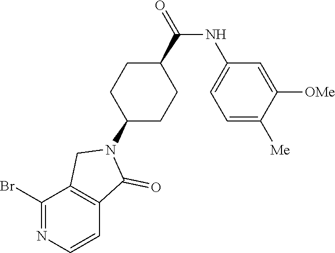 [SMILES: COc1cc(NC(=O)[C@H]2CC[C@@H](N3Cc4c(ccnc4Br)C3=O)CC2)ccc1C]

### (1s,4s)-4-(4-Bromo-1-oxo-1H-pyrrolo[3,4-c]pyridin-2(3H)-yl)-N-(3-methoxy-4-methylphenyl)cyclohexanecarboxamide

Following the synthetic procedure for Example 147, but starting with methyl 2-bromo-3-methylisonicotinate gave the title compound as a white solid. LCMS (ESI) m/z: 460.1 [M+H]⁺. ¹H NMR (400 MHz, CDCl₃) δ 8.52 (d, J=4.9 Hz, 1H), 7.70 (d, J=4.9 Hz, 1H), 7.33 (d, J=1.5 Hz, 1H), 7.20 (b, 1H), 7.07 (d, J=8.0 Hz, 1H), 6.80 (dd, J=7.9, 1.9 Hz, 1H), 4.40-4.30 (m, 3H), 3.87 (s, 3H), 2.69-2.67 (m, 1H), 2.29-2.25 (m, 2H), 2.18 (s, 3H), 2.14-2.02 (m, 2H), 1.92-1.78 (m, 4H).

### Example 177

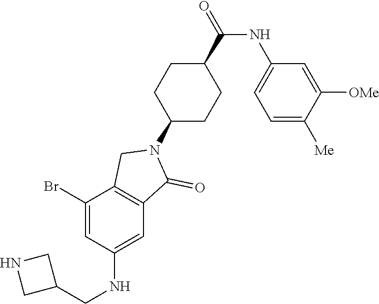 [SMILES: COc1cc(NC(=O)[C@H]2CC[C@@H](N3Cc4c(Br)cc(NCC5CNC5)cc4C3=O)CC2)ccc1C]

### (1s,4s)-4-(6-(Azetidin-3-ylmethylamino)-4-bromo-1-oxoisoindolin-2-yl)-N-(3-methoxy-4-methylphenyl)cyclohexanecarboxamide

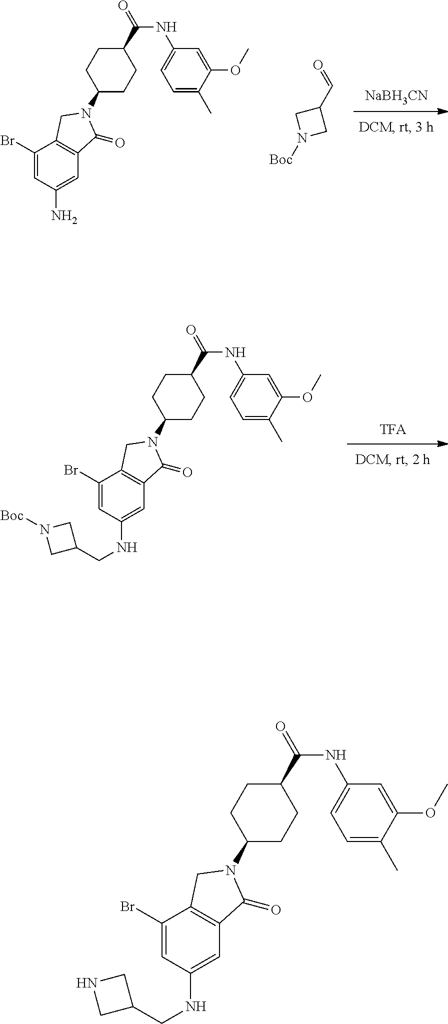 [SMILES: COc1cc(NC(=O)[C@H]2CC[C@@H](N3Cc4c(Br)cc(N)cc4C3=O)CC2)ccc1C ; CC(C)(C)OC(=O)N1CC(C=O)C1 ; "NaBH3CN" ; COc1cc(NC(=O)[C@H]2CC[C@@H](N3Cc4c(Br)cc(NCC5CN(C(=O)OC(C)(C)C)C5)cc4C3=O)CC2)ccc1C ; "TFA" ; COc1cc(NC(=O)[C@H]2CC[C@@H](N3Cc4c(Br)cc(NCC5CNC5)cc4C3=O)CC2)ccc1C]

### Step 1

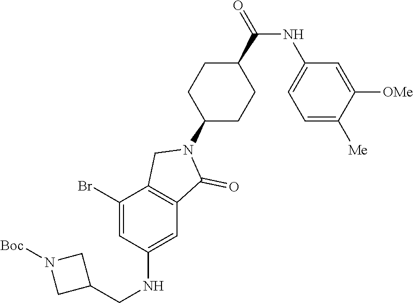 [SMILES: COc1cc(NC(=O)[C@H]2CC[C@@H](N3Cc4c(Br)cc(NCC5CN(C(=O)OC(C)(C)C)C5)cc4C3=O)CC2)ccc1C]

### tert-Butyl 3-((7-bromo-2-((1s,4s)-4-(3-methoxy-4-methylphenylcarbamoyl)cyclohexyl)-3-oxoisoindolin-5-ylamino)methyl)azetidine-1-carboxylate

To a solution of 4-(6-amino-4-bromo-1-oxo-isoindolin-2-yl)-N-(3-methoxy-4-methyl-phenyl)cyclohexanecarboxamide (50 mg, 110 μmol) and tert-butyl 3-formylazetidine-1-carboxylate (19.6 mg, 106 μmol) in DCM (3.0 mL) was added Sodium cyanoborohydride (33.3 mg, 529 μmol). The mixture was stirred at 25° C. for 3 hours. The solution was washed with H₂O, dried by Na₂SO₄, filtered and concentrated to give a crude oil. The crude product was purified by silica gel chromatography to give tert-butyl 3-((7-bromo-2-((1s,4s)-4-(3-methoxy-4-methylphenylcarbamoyl)cyclohexyl)-3-oxoisoindolin-5-ylamino)methyl)azetidine-1-carboxylate (40 mg, 62 μmol, 59% yield) as a yellow solid. LCMS ESI m/z: 585.2 [M+H−56]⁺.

### Step 2

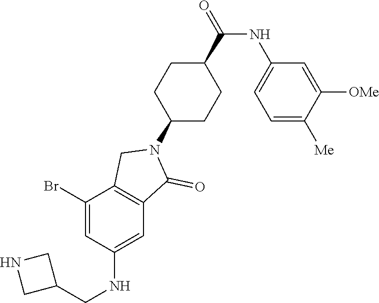 [SMILES: COc1cc(NC(=O)[C@H]2CC[C@@H](N3Cc4c(Br)cc(NCC5CNC5)cc4C3=O)CC2)ccc1C]

### (1s,4s)-4-(6-(Azetidin-3-ylmethylamino)-4-bromo-1-oxoisoindolin-2-yl)-N-(3-methoxy-4-methylphenyl)cyclohexanecarboxamide

To a solution of tert-butyl 3-((7-bromo-2-((1s,4s)-4-(3-methoxy-4-methylphenylcarbamoyl)cyclohexyl)-3-oxoisoindolin-5-ylamino)methyl)azetidine-1-carboxylate (40 mg, 62 μmol) in DCM (4.0 mL) was added TFA (3.0 g, 26 mmol, 2.0 mL). The mixture was stirred at 25° C. for 1 hour. The mixture was concentrated to give a crude oil. The crude product was purified by prep-HPLC to give (1s,4s)-4-(6-(azetidin-3-ylmethylamino)-4-bromo-1-oxoisoindolin-2-yl)-N-(3-methoxy-4-methylphenyl)cyclohexanecarboxamide (8.0 mg, 15 μmol, 24% yield) as a white solid. LCMS ESI m/z: 541.2 [M+H]⁺. ¹H NMR (400 MHz, MeOD-d₄) δ 7.29 (d, J=1.8 Hz, 1H), 7.05-7.00 (m, 2H), 6.98-6.89 (m, 2H), 4.31 (s, 2H), 4.26-4.18 (m, 1H), 3.94-3.73 (m, 5H), 3.61-3.52 (m, 2H), 3.37-2.33 (m, 2H), 3.17-3.00 (m, 1H), 2.74-2.71 (m, 1H), 2.23-2.21 (m, 2H), 2.16-2.07 (m, 5H), 1.90-1.79 (m, 2H), 1.79-1.71 (m, 2H).

### Example 178

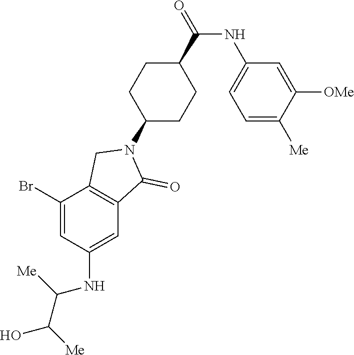 [SMILES: COc1cc(NC(=O)[C@H]2CC[C@@H](N3Cc4c(Br)cc(NC(C)C(C)O)cc4C3=O)CC2)ccc1C]

### (1s,4s)-4-(4-Bromo-6-(3-hydroxybutan-2-ylamino)-1-oxoisoindolin-2-yl)-N-(3-methoxy-4-methylphenyl)cyclohexanecarboxamide

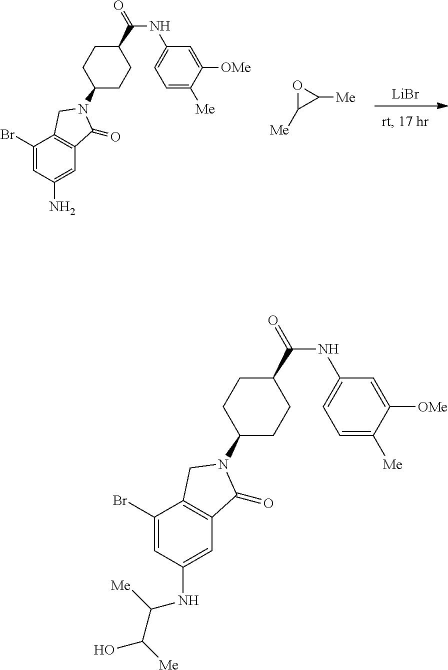 [SMILES: COc1cc(NC(=O)[C@H]2CC[C@@H](N3Cc4c(Br)cc(N)cc4C3=O)CC2)ccc1C ; CC1OC1C ; "LiBr" ; COc1cc(NC(=O)[C@H]2CC[C@@H](N3Cc4c(Br)cc(NC(C)C(C)O)cc4C3=O)CC2)ccc1C]

### Step 1

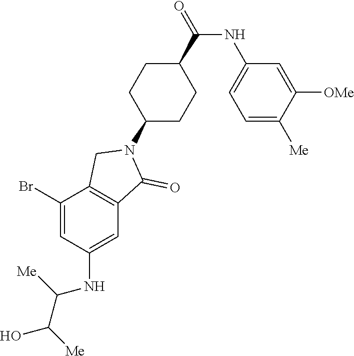 [SMILES: COc1cc(NC(=O)[C@H]2CC[C@@H](N3Cc4c(Br)cc(NC(C)C(C)O)cc4C3=O)CC2)ccc1C]

### (1s,4s)-4-(4-Bromo-6-(3-hydroxybutan-2-ylamino)-1-oxoisoindolin-2-yl)-N-(3-methoxy-4-methylphenyl)cyclohexanecarboxamide

To a solution of 4-(6-amino-4-bromo-1-oxo-isoindolin-2-yl)-N-(3-methoxy-4-methyl-phenyl)cyclohexanecarboxamide (30 mg, 64 μmol) in 2,3-dimethyloxirane (13.7 mg, 191 μmol) was added LiBr (552 μg, 6.35 μmol). The mixture was stirred at 25° C. for 17 hours. The mixture was filtered and the filtrate was concentrated to the crude, which was purified by prep-HPLC to give (1s,4s)-4-(4-bromo-6-(3-hydroxybutan-2-ylamino)-1-oxoisoindolin-2-yl)-N-(3-methoxy-4-methylphenyl)cyclohexanecarboxamide (4.7 mg, 8.6 μmol, 14% yield) as a white solid. LCMS ESI m/z 544.2 [M+H]⁺. ¹HNMR (400 MHz, MeOD-d₄) δ 7.29 (d, J=1.5 Hz, 1H), 7.07-7.00 (m, 2H), 6.95 (dd, J=8.4, 1.7 Hz, 2H), 4.57 (b, 1H), 4.30 (s, 2H), 4.25-4.20 (m, 1H), 3.89-3.74 (m, 4H), 3.52-3.40 (m, 1H), 2.73-2.71 (m, 1H), 2.27-2.04 (m, 7H), 1.97-1.65 (m, 4H), 1.17 (dd, J=6.4, 3.8 Hz, 6H).

### Example 179

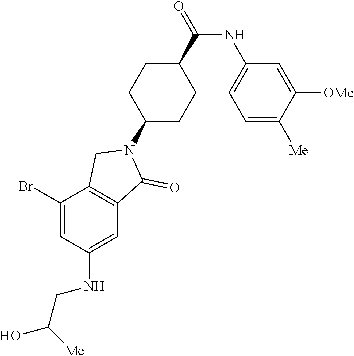 [SMILES: COc1cc(NC(=O)[C@H]2CC[C@@H](N3Cc4c(Br)cc(NCC(C)O)cc4C3=O)CC2)ccc1C]

### (1s,4s)-4-(4-Bromo-6-(2-hydroxypropylamino)-1-oxoisoindolin-2-yl)-N-(3-methoxy-4-methylphenyl)cyclohexanecarboxamide

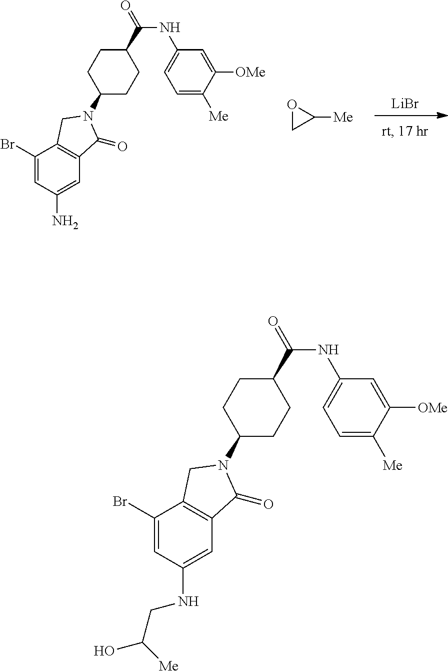

### Step 1

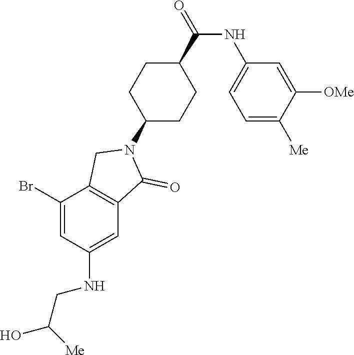 [SMILES: COc1cc(NC(=O)[C@H]2CC[C@@H](N3Cc4c(Br)cc(NCC(C)O)cc4C3=O)CC2)ccc1C]

### (1s,4s)-4-(4-Bromo-6-(2-hydroxypropylamino)-1-oxoisoindolin-2-yl)-N-(3-methoxy-4-methylphenyl)cyclohexanecarboxamide

To a solution of 4-(6-amino-4-bromo-1-oxo-isoindolin-2-yl)-N-(3-methoxy-4-methyl-phenyl)cyclohexanecarboxamide (30 mg, 64 μmol) in 2-methyloxirane (3.69 mg, 63.5 μmol, 4.44 μL) was added LiBr (552 μg, 6.35 μmol). The mixture was stirred at 25° C. for 17 hours. The mixture was filtered and the filtrate was concentrated to the crude, which was purified by prep-HPLC to give (1s,4s)-4-(4-bromo-6-(2-hydroxypropylamino)-1-oxoisoindolin-2-yl)-N-(3-methoxy-4-methylphenyl)cyclohexanecarboxamide (5.0 mg, 9.4 μmol, 15% yield) as a white solid. LCMS ESI m/z 530.2 [M+H]+. ¹HNMR (400 MHz, DMSO-d₆) δ 9.71 (s, 1H), 7.32 (d, J=1.5 Hz, 1H), 7.11 (dd, J=8.0, 1.7 Hz, 1H), 7.01 (d, J=7.8 Hz, 2H), 6.82 (d, J=1.8 Hz, 1H), 6.12 (t, J=5.8 Hz, 1H), 4.73 (d, J=4.6 Hz, 1H), 4.17 (s, 2H), 4.09-4.06 (m, 1H), 3.84-3.69 (m, 4H), 3.05-2.89 (m, 2H), 2.67-2.65 (m, 1H), 2.19-1.89 (m, 7H), 1.79-1.56 (m, 4H), 1.09 (d, J=6.2 Hz, 3H).

### Example 180

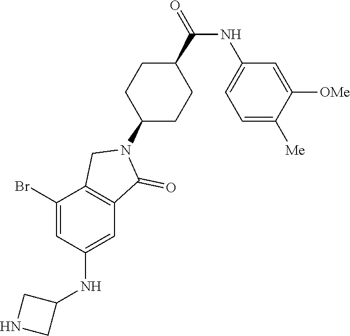 [SMILES: COc1cc(NC(=O)[C@H]2CC[C@@H](N3Cc4c(Br)cc(NC5CNC5)cc4C3=O)CC2)ccc1C]

### (1s,4s)-4-(6-(Azetidin-3-ylamino)-4-bromo-1-oxoisoindolin-2-yl)-N-(3-methoxy-4-methylphenyl)cyclohexanecarboxamide

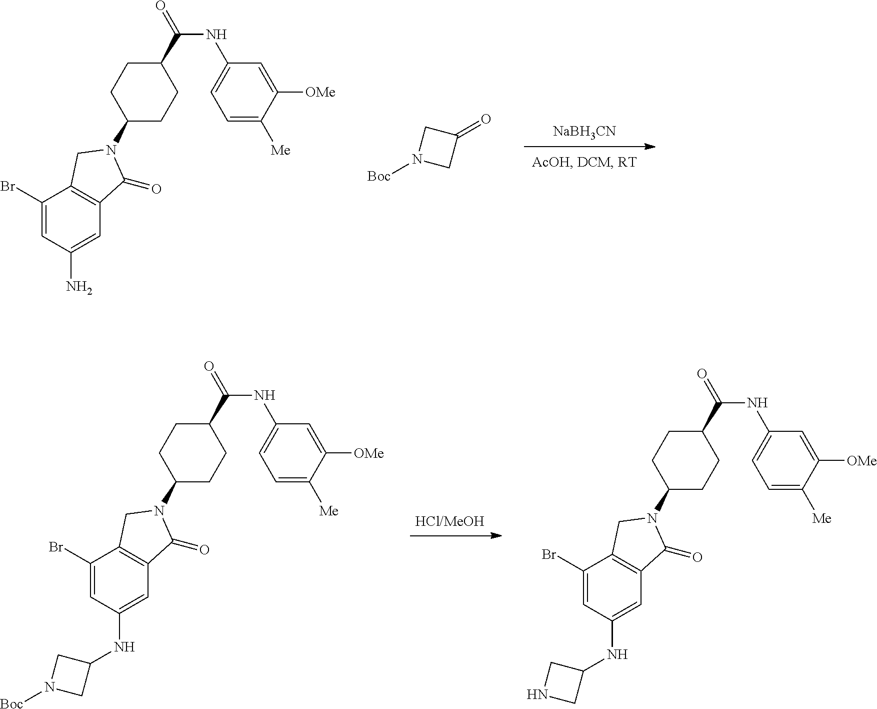 [SMILES: COc1cc(NC(=O)[C@H]2CC[C@@H](N3Cc4c(Br)cc(N)cc4C3=O)CC2)ccc1C ; CC(C)(C)OC(=O)N1CC(=O)C1 ; "NaBH3CN" ; COc1cc(NC(=O)[C@H]2CC[C@@H](N3Cc4c(Br)cc(NC5CN(C(=O)OC(C)(C)C)C5)cc4C3=O)CC2)ccc1C ; "HCl/MeOH" ; COc1cc(NC(=O)[C@H]2CC[C@@H](N3Cc4c(Br)cc(NC5CNC5)cc4C3=O)CC2)ccc1C]

### Step 1

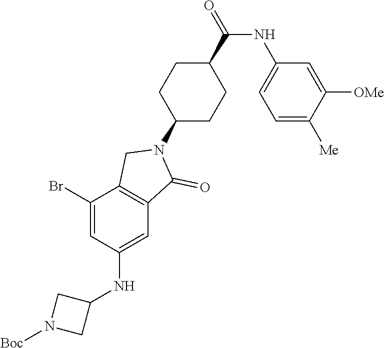 [SMILES: COc1cc(NC(=O)[C@H]2CC[C@@H](N3Cc4c(Br)cc(NC5CN(C(=O)OC(C)(C)C)C5)cc4C3=O)CC2)ccc1C]

### tert-Butyl 3-(7-bromo-2-((1s,4s)-4-(3-methoxy-4-methylphenylcarbamoyl)cyclohexyl)-3-oxoisoindolin-5-ylamino)azetidine-1-carboxylate

A solution of (1s,4s)-4-(6-amino-4-bromo-1-oxoisoindolin-2-yl)-N-(3-methoxy-4-methylphenyl)cyclohexanecarboxamide (60 mg, 130 μmol), tert-butyl 3-oxoazetidine-1-carboxylate (218 mg, 1.27 mmol) and acetic acid (15.3 mg, 254 μmol, 14.5 μL) in DCM (2 mL) was stirred at RT for 1 hour. Sodium cyanoborohydride (79.8 mg, 1.27 mmol) was added to the mixture, The resulting mixture was stirred at RT for another 16 hours. The mixture was diluted with water and extracted with EtOAc. The organic layers was dried over anhydrous Na₂SO₄, filtered and concentrated to get crude product, which was purified by silica gel chromatography to give tert-butyl 3-(7-bromo-2-((1s,4s)-4-(3-methoxy-4-methylphenylcarbamoyl)cyclohexyl)-3-oxoisoindolin-5-ylamino)azetidine-1-carboxylate (24 mg, 30% yield) as a yellow solid. LCMS (ESI) m/z: 571.2 [M−56+H]⁺.

### Step 2

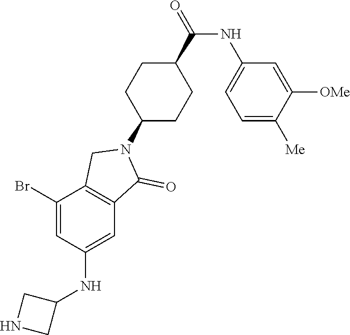 [SMILES: COc1cc(NC(=O)[C@H]2CC[C@@H](N3Cc4c(Br)cc(NC5CNC5)cc4C3=O)CC2)ccc1C]

### (1s,4s)-4-(6-(Azetidin-3-ylamino)-4-bromo-1-oxoisoindolin-2-yl)-N-(3-methoxy-4-methylphenyl)cyclohexanecarboxamide

A solution of tert-butyl 3-(7-bromo-2-((1s,4s)-4-(3-methoxy-4-methylphenylcarbamoyl)cyclohexyl)-3-oxoisoindolin-5-ylamino)azetidine-1-carboxylate (24 mg, 38 μmol) in HCl/MeOH (2 mL, 4M) was stirred at rt for 3 hours. The mixture was concentrated to get crude product, which was purified by prep-HPLC to give (1s,4s)-4-(6-(azetidin-3-ylamino)-4-bromo-1-oxoisoindolin-2-yl)-N-(3-methoxy-4-methylphenyl)cyclohexanecarboxamide (3.3 mg, 16% yield). LCMS (ESI) m/z: 528.5 [M+H]⁺. ¹H NMR (400 MHz, CD₃OD) δ 7.29 (d, J=1.8 Hz, 1H), 7.05-7.02 (m, 2H), 6.96 (dd, J=8.0, 1.7 Hz, 1H), 6.86 (d, J=1.9 Hz, 1H), 4.57-4.53 (m, 2H), 4.46-4.39 (m, 2H), 4.34 (s, 2H), 4.26-4.21 (m, 1H), 4.07-3.97 (m, 2H), 3.83 (s, 3H), 2.81-2.70 (m, 1H), 2.28-2.04 (m, 7H), 1.88-1.74 (m, 4H).

### Example 181

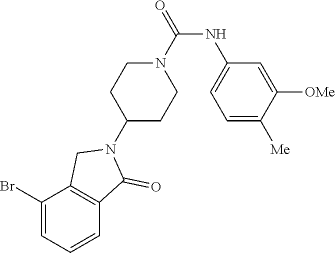 [SMILES: COc1cc(NC(=O)N2CCC(N3Cc4c(Br)cccc4C3=O)CC2)ccc1C]

### 4-(4-Bromo-1-oxoisoindolin-2-yl)-N-(3-methoxy-4-methylphenyl)piperidine-1-carboxamide

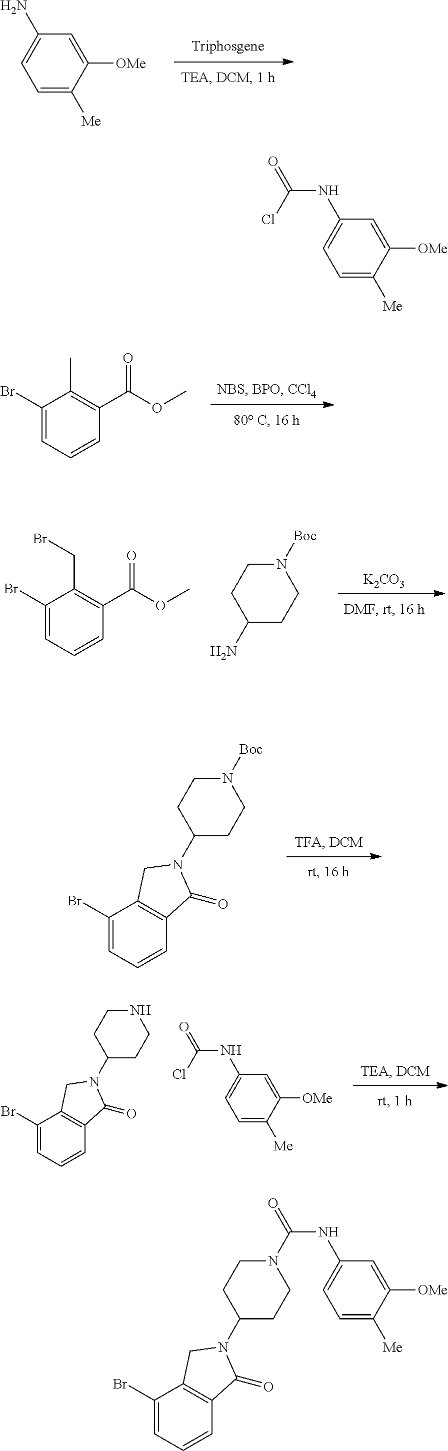

### Intermediate

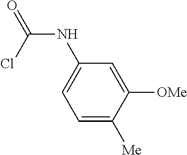 [SMILES: COc1cc(NC(=O)Cl)ccc1C]

### 3-Methoxy-4-methylphenylcarbamic chloride

A solution of 3-methoxy-4-methyl-aniline (0.030 g, 220 μmol), bis(trichloromethyl)carbonate (52 mg, 170 μmol) and N,N-diethylethanamine (44 mg, 440 μmol, 61 μL) in DCM (10 mL) was stirred for 1 hour at 25° C. The solvent was evaporated in vacuo to dryness to get 3-methoxy-4-methylphenylcarbamic chloride (0.043 g, 220 μmol, 98% yield) as a white solid. The residue used directly to the next step.

### Step 1 and 2

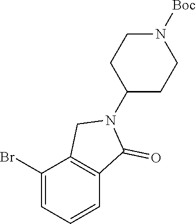 [SMILES: CC(C)(C)OC(=O)N1CCC(N2Cc3c(Br)cccc3C2=O)CC1]

### tert-Butyl 4-(4-bromo-1-oxoisoindolin-2-yl)piperidine-1-carboxylate

Following the synthetic procedure for Example 147, but starting with methyl 3-bromo-2-methylbenzoate gave the title compound as a white solid. LCMS (ESI) m/z: 341.0 [M−56+H]⁺.

### Step 3

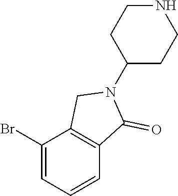 [SMILES: O=C1c2cccc(Br)c2CN1C1CCNCC1]

### 4-Bromo-2-(piperidin-4-yl)isoindolin-1-one

A solution of tert-butyl 4-(4-bromo-1-oxo-isoindolin-2-yl)piperidine-1-carboxylate (0.30 g, 760 μmol) in TFA/DCM (1/4, 10 mL) was stirred for 16 hours at 25° C. The reaction was concentrated. To the resulting residue was added EtOAc (70 mL) and washed with sat. NaHCO₃ aq. (3×50 mL). The combined organic extracts were dried over Na₂SO₄ and evaporated in vacuo. The residue was purified by flash chromatography on silica gel (0-20% MeOH in DCM) to get 4-bromo-2-(4-piperidyl)isoindolin-1-one (0.18 g, 610 μmol, 80.4% yield) as a white solid. LCMS ESI m/z: 297.0 [M+H]⁺.

### Step 4

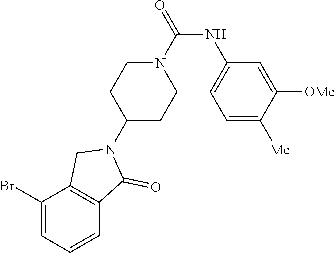 [SMILES: COc1cc(NC(=O)N2CCC(N3Cc4c(Br)cccc4C3=O)CC2)ccc1C]

### 4-(4-Bromo-1-oxoisoindolin-2-yl)-N-(3-methoxy-4-methylphenyl) piperidine-1-carboxamide

A solution of 4-bromo-2-(4-piperidyl)isoindolin-1-one (0.050 g, 170 μmol), N-(3-methoxy-4-methyl-phenyl)carbamoyl chloride (34 mg, 170 μmol) and N,N-diethylethanamine (17 mg, 170 μmol, 24 μL) in DCM (10 mL) was stirred for 1 hour at 25° C. The solvent was evaporated in vacuo to dryness. The residue was purified by Prep-HPLC to get 4-(4-bromo-1-oxoisoindolin-2-yl)-N-(3-methoxy-4-methylphenyl) piperidine-1-carboxamide (0.0073 g, 16 μmol, 9.4% yield) as a white solid. LCMS ESI m/z: 460.1 [M+H]⁺. ¹H NMR (400 MHz, CDCl₃) δ 7.85-7.78 (m, 1H), 7.67 (dd, J=8.0, 0.8 Hz, 1H), 7.38 (t, J=7.6 Hz, 1H), 7.20 (d, J=2.0 Hz, 1H), 7.02 (d, J=8.0 Hz, 1H), 6.64 (dd, J=8.0, 2.0 Hz, 1H), 6.38 (s, 1H), 4.56-4.45 (m, 1H), 4.30-4.15 (m, 4H), 3.83 (s, 3H), 3.16-2.98 (m, 2H), 2.16 (s, 3H), 2.04-1.90 (m, 2H), 1.90-1.70 (m, 2H).

### Example 182

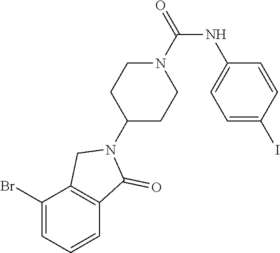 [SMILES: O=C(Nc1ccc(I)cc1)N1CCC(N2Cc3c(Br)cccc3C2=O)CC1]

### 4-(4-Bromo-1-oxoisoindolin-2-yl)-N-(4-iodophenyl) piperidine-1-carboxamide

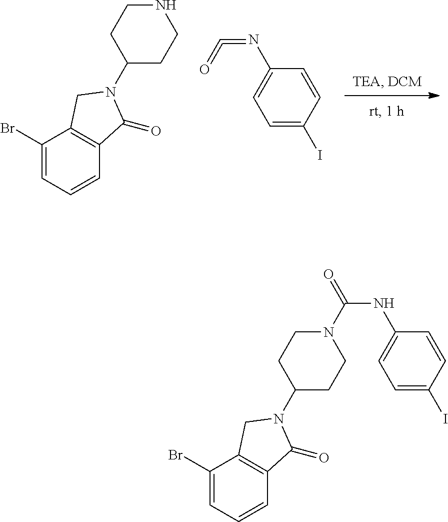

### Step 1

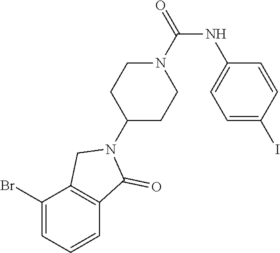 [SMILES: O=C(Nc1ccc(I)cc1)N1CCC(N2Cc3c(Br)cccc3C2=O)CC1]

### 4-(4-Bromo-1-oxoisoindolin-2-yl)-N-(4-iodophenyl) piperidine-1-carboxamide

A solution of 4-bromo-2-(4-piperidyl)isoindolin-1-one (0.030 g, 100 μmol), 1-iodo-4-isocyanato-benzene (24.9 mg, 102 μmol) and N,N-diethylethanamine (20.6 mg, 203 μmol, 28.3 μL) in DCM (5 mL) was stirred for 1 hour at 25° C. To the reaction mixture was added EtOAc (50 mL) and washed with H₂O (3×50 mL). The combined organic extracts were dried over Na₂SO₄ and evaporated in vacuo. The residue was purified by Prep-HPLC to get 4-(4-bromo-1-oxoisoindolin-2-yl)-N-(4-iodophenyl) piperidine-1-carboxamide (0.0055 g, 10 μmol, 10% yield) as a white solid. LCMS ESI m/z: 542.0 [M+H]⁺. ¹H NMR (400 MHz, CDCl₃) δ 7.81 (d, J=7.6 Hz, 1H), 7.71-7.67 (m, 1H), 7.67-7.53 (m, 2H), 7.38 (t, J=7.6 Hz, 1H), 7.25-7.10 (m, 2H), 6.45-6.35 (m, 1H), 4.56-4.45 (m, 1H), 4.30-4.14 (m, 4H), 3.08 (t, J=12.8 Hz, 2H), 2.00-1.90 (m, 2H), 1.90-1.75 (m, 2H).

### Example 183

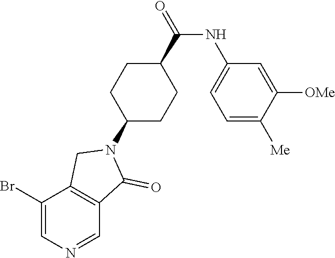 [SMILES: COc1cc(NC(=O)[C@H]2CC[C@@H](N3Cc4c(Br)cncc4C3=O)CC2)ccc1C]

### (1s,4s)-4-(7-bromo-3-oxo-1H-pyrrolo[3,4-c]pyridin-2(3H)-yl)-N-(3-methoxy-4-methylphenyl)cyclohexanecarboxamide

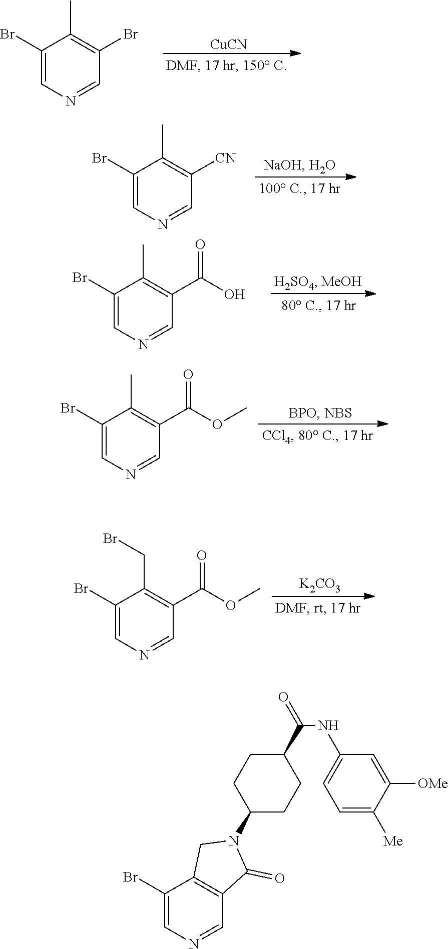

### Step 1

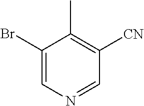

### 5-Bromo-4-methylnicotinonitrile

To a solution of 3,5-dibromo-4-methyl-pyridine (13 g, 52 mmol) in DMF (100 mL) was added CuCN (4.85 g, 51.8 mmol). The mixture was stirred at 150° C. for 17 hours. The mixture was poured into EtOAc (100 mL) washed with water (50 mL*3). The organic phase was concentrated in vacuum. The residue was purified by silica column (PE:EtOAc=30:1 to 1:1) to give 5-bromo-4-methylnicotinonitrile (5.0 g, 25 mmol, 49% yield) as a yellow solid. LCMS ESI m/z 198.9 [M+H]⁺.

### Step 2

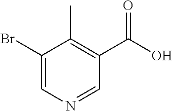

### 5-Bromo-4-methylnicotinic Acid

A mixture of 5-bromo-4-methyl-pyridine-3-carbonitrile (200 mg, 1.02 mmol) and sodium hydroxide (41 mg, 1.0 mmol) in H₂O (8 mL) was stirred at 100° C. for 17 hours. The mixture was concentrated in vacuo, and the residue was used directly in the next step without purification. LCMS ESI m/z 217.9 [M+H]⁺.

### Step 3

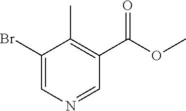

### Methyl 5-bromo-4-methylnicotinate

To a solution of 5-bromo-4-methyl-pyridine-3-carboxylic acid (1.0 g, 4.6 mmol) in Methanol (1 mL) was added H₂SO₄ (1.36 g, 13.9 mmol). The mixture was stirred at 80° C. for 17 hours. The mixture was poured into sat. NaHCO₃ aq. (100 mL) and extracted with EtOAc (50 mL*3). The organic phases were concentrated in vacuo. The residue was used directly in the next step without purification. LCMS ESI m/z 231.9 [M+H]⁺.

### Step 4 and 5

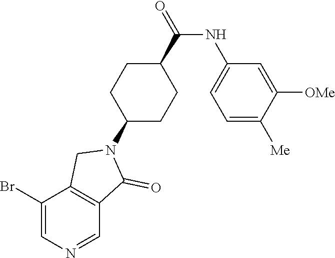 [SMILES: COc1cc(NC(=O)[C@H]2CC[C@@H](N3Cc4c(Br)cncc4C3=O)CC2)ccc1C]

### (1s,4s)-4-(7-Bromo-3-oxo-1H-pyrrolo[3,4-c]pyridin-2(3H)-yl)-N-(3-methoxy-4-methylphenyl)cyclohexanecarboxamide

Following the synthetic procedure for Example 147, but starting with methyl 5-bromo-4-methylnicotinate gave the title compound as a white solid. LCMS (ESI) m/z: 457.7 [M+H]⁺. ¹H NMR (400 MHz, DMSO-d₆) δ9.71 (s, 1H), 8.87 (d, J=4.0 Hz, 2H), 7.33 (d, J=1.7 Hz, 1H), 7.11 (dd, J=8.0, 1.8 Hz, 1H), 7.02 (d, J=8.4 Hz, 1H), 4.50 (s, 2H), 4.19-4.02 (m, 1H), 3.75 (s, 3H), 2.68-2.66 (m, 1H), 2.18-1.93 (m, 7H), 1.79-1.56 (m, 4H).

### Example 184

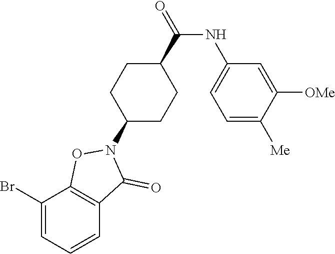 [SMILES: COc1cc(NC(=O)[C@H]2CC[C@@H](n3oc4c(Br)cccc4c3=O)CC2)ccc1C]

### (1s,4s)-4-(7-Bromo-3-oxobenzo[d]isoxazol-2(3H)-yl)-N-(3-methoxy-4-methylphenyl)cyclohexanecarboxamide

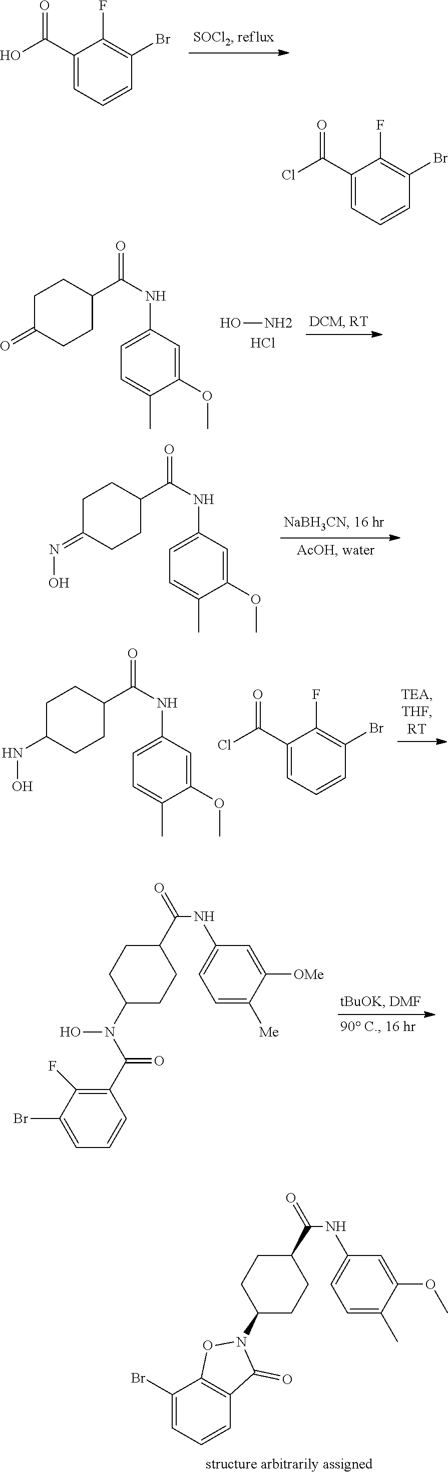

### Intermediate

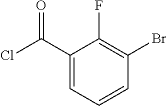 [SMILES: O=C(Cl)c1cccc(Br)c1F]

### 3-Bromo-2-fluorobenzoyl Chloride

A solution of 3-bromo-2-fluoro-benzoic acid (230 mg, 1.05 mmol) in SOCl₂ (5 mL) was stirred at 90° C. for 2 hours. The reaction mixture was concentrated to the crude and directly used to the next step without further purification.

### Step 1

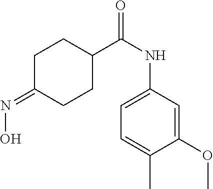 [SMILES: COc1cc(NC(=O)C2CCC(=NO)CC2)ccc1C]

### 4-(Hydroxyimino)-N-(3-methoxy-4-methylphenyl)cyclohexanecarboxamide

A mixture of N-(3-methoxy-4-methyl-phenyl)-4-oxo-cyclohexanecarboxamide (1.8 g, 6.9 mmol) and hydroxylamine; hydrochloride (527 mg, 7.58 mmol) in DCM (15 mL) was stirred at 25° C. for 16 hours. After the reaction was completed, the reaction was filtered and the solids was collected to give 4-(hydroxyimino)-N-(3-methoxy-4-methylphenyl)cyclohexanecarboxamide (1.2 g, 4.3 mmol, 63% yield) as a white solid. LCMS (ESI) m/z: 277.2 [M+H]⁺.

### Step 2

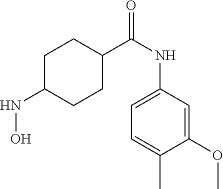 [SMILES: COc1cc(NC(=O)C2CCC(NO)CC2)ccc1C]

### 4-(Hydroxyamino)-N-(3-methoxy-4-methylphenyl)cyclohexanecarboxamide

A solution of 4-hydroxyimino-N-(3-methoxy-4-methyl-phenyl)cyclohexanecarboxamide (1.2 g, 4.3 mmol) in AcOH (12 mL) and water (4 mL) was stirred at 25° C., then added sodium cyanoborohydride (546 mg, 8.69 mmol) in portions. The reaction mixture was stirred at 25° C. for 16 hours. Then poured into water and basified by 1.0 M NaOH solution, extracted with EtOAc, dried and concentrated to give 4-(hydroxyamino)-N-(3-methoxy-4-methylphenyl)cyclohexanecarboxamide (500 mg, 1.80 mmol, 41% yield) as a white solid. LCMS (ESI) m/z: 279.2 [M+H]⁺.

### Step 3

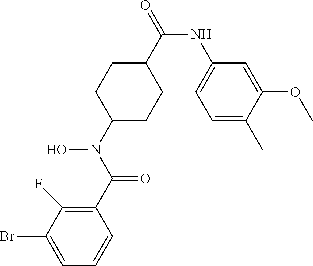 [SMILES: COc1cc(NC(=O)C2CCC(N(O)C(=O)c3cccc(Br)c3F)CC2)ccc1C]

### 3-Bromo-2-fluoro-N-hydroxy-N-(4-(3-methoxy-4-methylphenylcarbamoyl)cyclohexyl)benzamide

A solution of 4-(hydroxyamino)-N-(3-methoxy-4-methylphenyl)cyclohexanecarboxamide (270 mg, 970 μmol) and N,N-diethylethanamine (196 mg, 1.94 mmol, 270 μL) in THF (5 mL) was added dropwise a solution of 3-bromo-2-fluoro-benzoyl chloride (230 mg, 970 μmol) in THF (5 mL) at 0° C. The reaction mixture was stirred at 25° C. for 1 hour. The resulting mixture was poured into water and extracted with EtOAc. The organic layers were dried and concentrated to the crude 3-bromo-2-fluoro-N-hydroxy-N-[4-[(3-methoxy-4-methyl-phenyl)carbamoyl]cyclohexyl]benzamide (250 mg, 522 μmol, 53.8% yield) as a light yellow solid. LCMS (ESI) m/z: 479.1 [M+H]⁺.

### Step 4

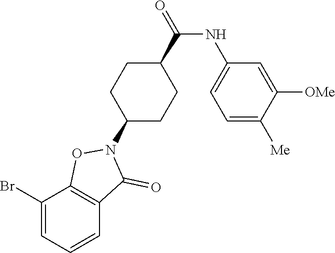 [SMILES: COc1cc(NC(=O)[C@H]2CC[C@@H](n3oc4c(Br)cccc4c3=O)CC2)ccc1C]

### (1s,4s)-4-(7-Bromo-3-oxobenzo[d]isoxazol-2(3H)-yl)-N-(3-methoxy-4-methylphenyl)cyclohexanecarboxamide

A mixture of 3-bromo-2-fluoro-N-hydroxy-N-[4-[(3-methoxy-4-methyl-phenyl)carbamoyl]cyclohexyl]benzamide (100 mg, 209 μmol) and tBuOK (46.8 mg, 417 μmol) in THF (10 mL) was stirred at 90° C. for 16 hour. The reaction mixture was poured into water (30 mL) and extracted with EtOAc (30 mL*3). The organic layers were dried and concentrated. The crude was purified by Prep-HPLC to give (1s,4s)-4-(7-bromo-3-oxobenzo[d]isoxazol-2(3H)-yl)-N-(3-methoxy-4-methylphenyl)cyclohexanecarboxamide (16 mg, 34.8 μmol, 16.7% yield) as a white solid. The compound structure was arbitrarily assigned (1s,4s). LCMS (ESI) m/z: 458.6 [M+H]⁺. ¹H NMR (400 MHz, CD₃OD) δ 7.55 (dd, J=7.8, 1.5 Hz, 1H), 7.38 (dd, J=8.0, 1.5 Hz, 1H), 6.98 (d, J=7.8 Hz, 1H), 6.75 (d, J=1.9 Hz, 1H), 6.66 (dd, J=7.8, 1.9 Hz, 1H), 6.60 (t, J=7.9 Hz, 1H), 4.57-4.54 (m, 1H), 3.74 (s, 3H), 2.74-2.66 (m, 1H), 2.20-1.85 (m, 11H).

### Example 185

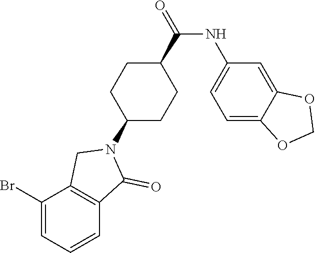 [SMILES: O=C1c2cccc(Br)c2CN1[C@H]1CC[C@@H](C(=O)Nc2ccc3c(c2)OCO3)CC1]

### (1s,4s)-N-(Benzo[d][1,3]dioxol-5-yl)-4-(4-bromo-1-oxoisoindolin-2-yl)cyclohexanecarboxamide

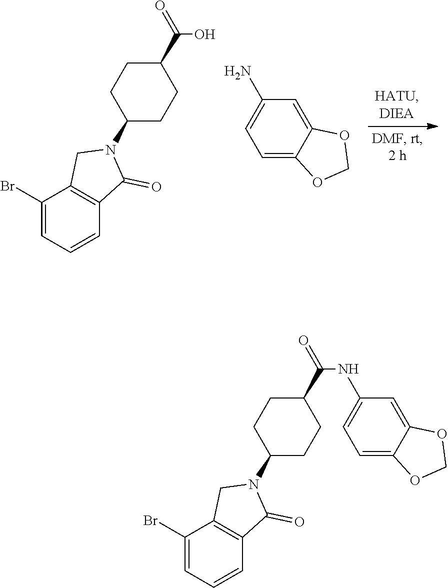 [SMILES: O=C1c2cccc(Br)c2CN1[C@H]1CC[C@@H](C(=O)O)CC1 ; Nc1ccc2c(c1)OCO2 ; O=C1c2cccc(Br)c2CN1[C@H]1CC[C@@H](C(=O)Nc2ccc3c(c2)OCO3)CC1]

### Step 1

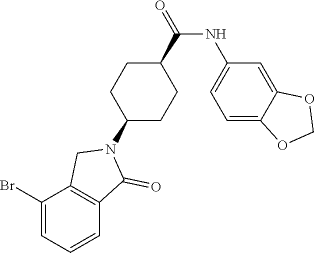 [SMILES: O=C1c2cccc(Br)c2CN1[C@H]1CC[C@@H](C(=O)Nc2ccc3c(c2)OCO3)CC1]

### (1s,4s)-N-(Benzo[d][1,3]dioxol-5-yl)-4-(4-bromo-1-oxoisoindolin-2-yl)cyclohexanecarboxamide

To a solution of 4-(4-bromo-1-oxo-isoindolin-2-yl)cyclohexanecarboxylic acid (40 mg, 120 μmol) in DMF (3 mL) was added HATU (54 mg, 140 μmol) and DIPEA (46 mg, 349 μmol). 30 mins later, benzo[d][1,3]dioxol-5-amine (19 mg, 140 μmol) was added. The mixture was stirred at 25° C. for 2 hours. The mixture was poured into EtOAc (20 mL) and washed with water (10 mL*3). The organic extracts were washed with brine, dried by Na₂SO₄, filtered and concentrated to give a crude oil. The crude product was purified by prep-HPLC to give (1s,4s)-N-(benzo[d][1,3]dioxol-5-yl)-4-(4-bromo-1-oxoisoindolin-2-yl)cyclohexanecarboxamide (10 mg, 22 μmol, 18% yield) as a brown solid. LCMS ESI m/z: 456.6 [M+H]⁺. 1H NMR (400 MHz, DMSO-d₆) δ 9.70 (s, 1H), 7.81 (dd, J=7.9, 0.8 Hz, 1H), 7.70 (dd, J=7.5, 0.7 Hz, 1H), 7.47 (t, J=7.7 Hz, 1H), 7.34 (d, J=2.0 Hz, 1H), 7.00 (dd, J=8.4, 2.1 Hz, 1H), 6.84 (d, J=8.4 Hz, 1H), 5.97 (s, 2H), 4.37 (s, 2H), 4.17-4.05 (m, 1H), 2.65-2.63 (m, 1H), 2.16-1.94 (m, 4H), 1.76-1.58 (m, 4H).

### Example 186

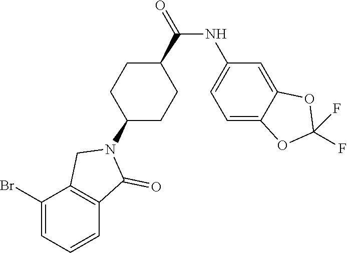 [SMILES: O=C1c2cccc(Br)c2CN1[C@H]1CC[C@@H](C(=O)Nc2ccc3c(c2)OC(F)(F)O3)CC1]

### (1s,4s)-4-(4-Bromo-1-oxoisoindolin-2-yl)-N-(2,2-difluorobenzo[d][1,3]dioxol-5-yl)cyclohexanecarboxamide

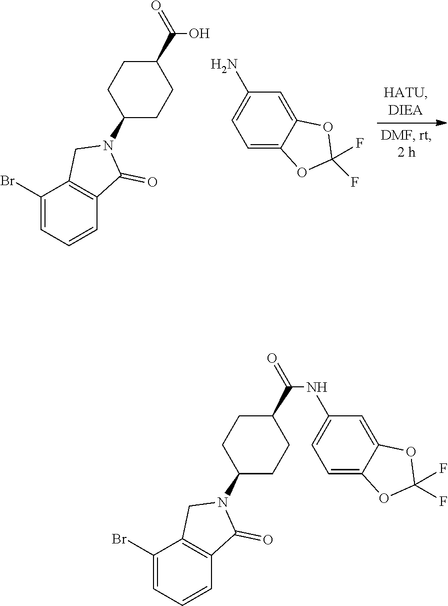

### Step 1

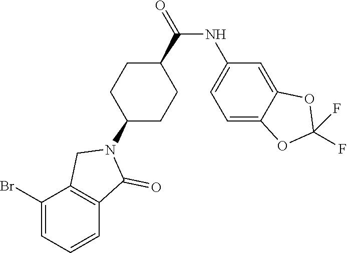 [SMILES: O=C1c2cccc(Br)c2CN1[C@H]1CC[C@@H](C(=O)Nc2ccc3c(c2)OC(F)(F)O3)CC1]

### (1s,4s)-4-(4-Bromo-1-oxoisoindolin-2-yl)-N-(2,2-difluorobenzo[d][1,3]dioxol-5-yl)cyclohexanecarboxamide

Following the synthetic procedure for Example 185, but starting with 2,2-difluorobenzo[d][1,3]dioxol-5-amine gave the title compound as a white solid. LCMS (ESI) m/z: 494.5 [M+H]⁺. ¹H NMR (400 MHz, DMSO-d₆) δ 10.03 (s, 1H), 7.90-7.75 (m, 2H), 7.70 (dd, J=7.5, 0.7 Hz, 1H), 7.47 (t, J=7.7 Hz, 1H), 7.36-7.25 (m, 2H), 4.37 (s, 2H), 4.16-4.06 (m, 1H), 2.70-2.68 (m, 1H), 2.14-2.11 (m, 2H), 2.05-1.93 (m, 2H), 1.80-1.60 (m, 4H).

### Example 187

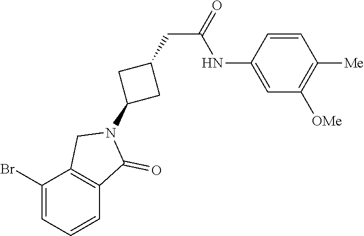 [SMILES: COc1cc(NC(=O)C[C@H]2C[C@H](N3Cc4c(Br)cccc4C3=O)C2)ccc1C]

### 2-((1r,3r)-3-(4-Bromo-1-oxoisoindolin-2-yl)cyclobutyl)-N-(3-methoxy-4-methylphenyl)acetamide

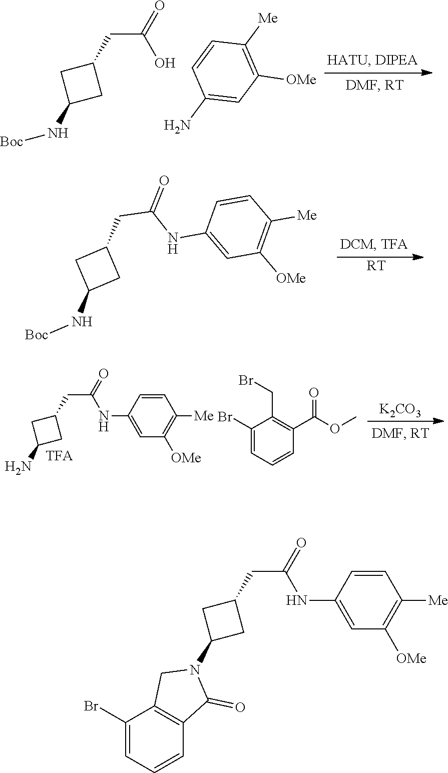

### Step 1

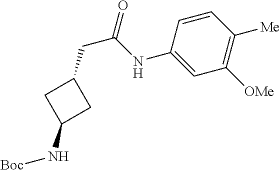 [SMILES: COc1cc(NC(=O)C[C@H]2C[C@H](NC(=O)OC(C)(C)C)C2)ccc1C]

### tert-Butyl (1r,3r)-3-(2-(3-methoxy-4-methylphenylamino)-2-oxoethyl)cyclobutylcarbamate

To a solution of 2-[3-(tert-butoxycarbonylamino)cyclobutyl]acetic acid (500 mg, 2.18 mmol) was added HATU (1.00 g, 2.62 mmol) and N-ethyl-N-isopropyl-propan-2-amine (846 mg, 6.54 mmol, 1.14 mL) and DMF (10 mL). The resulting mixture was stirred at room temperature for 0.5 hours. To the mixture was added 3-methoxy-4-methyl-aniline (299.16 mg, 2.18 mmol) and the reaction was stirred at rt for 3 hours. The mixture was diluted with water and extracted with EtOAc. The organic layers were dried over anhydrous Na₂SO₄, filtered and concentrated to get crude product, which was purified by silica gel chromatography to give tert-butyl (1r,3r)-3-(2-(3-methoxy-4-methylphenylamino)-2-oxoethyl)cyclobutylcarbamate (200 mg, 26% yield). LCMS (ESI) m/z: 292.9 [M−56+H]⁺.

### Step 2

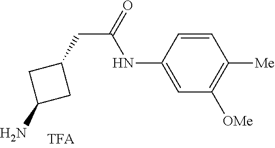 [SMILES: COc1cc(NC(=O)C[C@H]2C[C@H](N)C2)ccc1C]

### 2-((1r,3r)-3-Aminocyclobutyl)-N-(3-methoxy-4-methylphenyl)acetamide

A solution of tert-butyl (1r,3r)-3-(2-(3-methoxy-4-methylphenylamino)-2-oxoethyl)cyclobutylcarbamate (200 mg, 570 μmol) in TFA/DCM (1/3, 5 mL) was stirred at rt for 1 hour. The reaction mixture was concentrated under reduced pressure to give 2-((1r,3r)-3-aminocyclobutyl)-N-(3-methoxy-4-methylphenyl)acetamide as a crude. The crude was use directly to the next step. LCMS (ESI) m/z: 248.9 [M+H]⁺.

### Step 3

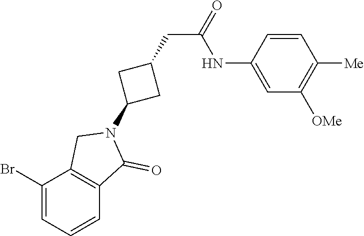 [SMILES: COc1cc(NC(=O)C[C@H]2C[C@H](N3Cc4c(Br)cccc4C3=O)C2)ccc1C]

### 2-((1r,3r)-3-(4-Bromo-1-oxoisoindolin-2-yl)cyclobutyl)-N-(3-methoxy-4-methylphenyl)acetamide

To a solution of 2-((1r,3r)-3-aminocyclobutyl)-N-(3-methoxy-4-methylphenyl)acetamide (200 mg, 805 μmol) was added K₂CO₃ (334 mg, 2.42 mmol) and DMF (5 mL). The resulting mixture was stirred at rt for 16 hours. The reaction mixture was diluted with water (20 mL) and extracted with EtOAc (30 mL). The organic layer was dried over anhydrous Na₂SO₄, filtered and concentrated to get crude product, which was purified by prep-HPLC to give 2-((1r,3r)-3-(4-bromo-1-oxoisoindolin-2-yl)cyclobutyl)-N-(3-methoxy-4-methylphenyl)acetamide (7.4 mg, 2.0% yield) as a white solid. LCMS (ESI) m/z: 445.6 [M+H]⁺. ¹H NMR (400 MHz, DMSO-d₆) δ 9.90 (s, 1H), 7.82 (d, J=7.9 Hz, 1H), 7.69 (d, J=7.0 Hz, 1H), 7.46 (t, J=7.7 Hz, 1H), 7.32 (d, J=1.4 Hz, 1H), 7.07-6.99 (m, 2H), 5.03-4.94 (m, 1H), 4.57 (s, 2H), 3.74 (s, 3H), 2.63-2.57 (m, 5H), 2.08-2.05 (m, 5H).

### Example 188

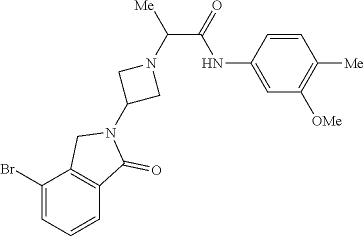 [SMILES: COc1cc(NC(=O)C(C)N2CC(N3Cc4c(Br)cccc4C3=O)C2)ccc1C]

### 2-(3-(4-Bromo-1-oxoisoindolin-2-yl)azetidin-1-yl)-N-(3-methoxy-4-methylphenyl)propanamide

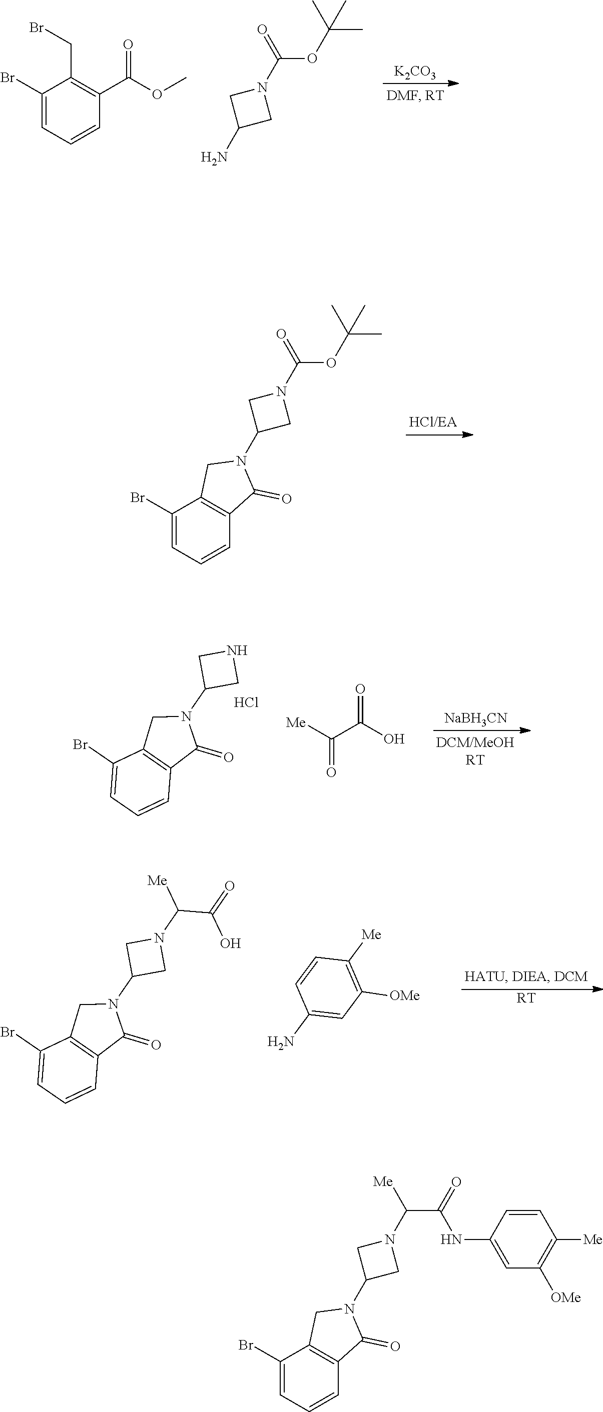

### Step 1

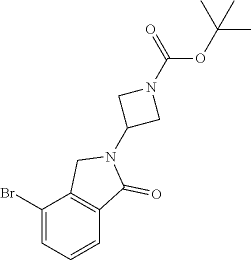 [SMILES: CC(C)(C)OC(=O)N1CC(N2Cc3c(Br)cccc3C2=O)C1]

### tert-Butyl 3-(4-bromo-1-oxoisoindolin-2-yl)azetidine-1-carboxylate

Following the synthetic procedure for Example 187, but starting with tert-butyl 3-aminoazetidine-1-carboxylate gave the title compound as a white solid. LCMS (ESI) m/z: 313.0 [M−56+H]⁺.

### Step 2

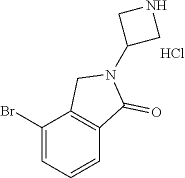 [SMILES: O=C1c2cccc(Br)c2CN1C1CNC1 ; Cl]

### 2-(Azetidin-3-yl)-4-bromoisoindolin-1-one hydrochloride

To a solution of tert-butyl 3-(4-bromo-1-oxo-isoindolin-2-yl)azetidine-1-carboxylate (830 mg, 2.26 mmol) in Ethyl acetate (3 mL) was added HCl-EtOAc (3 mL, 4M). The reaction mixture was stirred at room temperature for 3 hours. The reaction mixture was concentrated to give 2-(azetidin-3-yl)-4-bromoisoindolin-1-one hydrochloride as a crude product, which was used to the next step without further purification. LCMS (ESI) m/z: 267.0 [M+H]⁺.

### Step 3

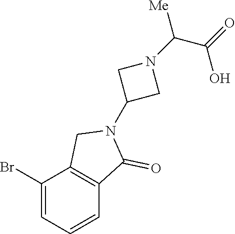 [SMILES: CC(C(=O)O)N1CC(N2Cc3c(Br)cccc3C2=O)C1]

### 2-(3-(4-Bromo-1-oxoisoindolin-2-yl)azetidin-1-yl)propanoic Acid

To a solution of 2-(azetidin-3-yl)-4-bromoisoindolin-1-one hydrochloride (300 mg, 1.12 mmol) and 2-oxopropanoic acid (989 mg, 11.2 mmol) in DCM/MeOH (2/1, 9 mL) was added sodium cyanoborohydride (423 mg, 6.74 mmol). The reaction mixture was stirred at room temperature for 2 hours. The reaction mixture was worked up with water and EtOAc. The organic layers dried and concentrated. The residue was purified by Prep-HPLC to give 2-(3-(4-bromo-1-oxoisoindolin-2-yl)azetidin-1-yl)propanoic acid (70 mg, 18% yield) as a white solid. LCMS (ESI) m/z: 340.7 [M+H]⁺.

### Step 4

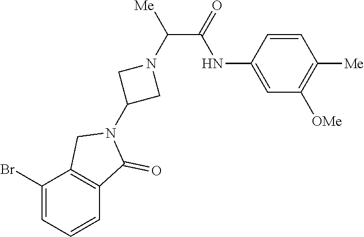 [SMILES: COc1cc(NC(=O)C(C)N2CC(N3Cc4c(Br)cccc4C3=O)C2)ccc1C]

### 2-(3-(4-Bromo-1-oxoisoindolin-2-yl)azetidin-1-yl)-N-(3-methoxy-4-methylphenyl) propanamide

To a solution of 2-(3-(4-bromo-1-oxoisoindolin-2-yl)azetidin-1-yl) propanoic acid (50 mg, 150 μmol) and 3-methoxy-4-methyl-aniline (40 mg, 290 μmol) in DCM (3 mL) was added DIPEA (38 mg, 290 μmol) and HATU (84 mg, 220 μmol). The reaction mixture was stirred at RT for 3 hours. The reaction system was worked up with water and DCM, and then the organic layers were dried and purified by Prep-HPLC to give 2-(3-(4-bromo-1-oxoisoindolin-2-yl)azetidin-1-yl)-N-(3-methoxy-4-methylphenyl) propanamide (31 mg, 67 μmol, 46% yield, 99.6%) as a white solid. LCMS (ESI) m/z: 457.6 [M+H]⁺. ¹H NMR (400 MHz, DMSO-d₆) δ 9.57 (s, 1H), 7.85 (dd, J=7.9, 0.7 Hz, 1H), 7.71 (d, J=7.4 Hz, 1H), 7.48 (t, J=7.7 Hz, 1H), 7.38 (d, J=1.8 Hz, 1H), 7.20 (dd, J=8.0, 1.9 Hz, 1H), 7.04 (d, J=8.4 Hz, 1H), 4.95-4.91 (m, 1H), 4.65 (s, 2H), 3.77 (s, 3H), 3.64-3.60 (m, 2H), 3.54-3.46 (m, 2H), 3.14 (q, J=6.8 Hz, 1H), 2.09 (s, 3H), 1.18 (d, J=6.7 Hz, 3H).

### Example 189

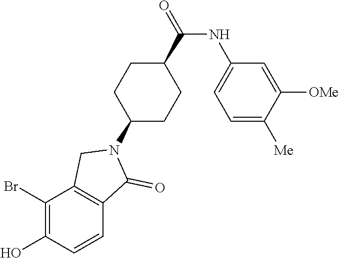 [SMILES: COc1cc(NC(=O)[C@H]2CC[C@@H](N3Cc4c(ccc(O)c4Br)C3=O)CC2)ccc1C]

### (1s,4s)-4-(4-Bromo-5-hydroxy-1-oxoisoindolin-2-yl)-N-(3-methoxy-4-methylphenyl)cyclohexanecarboxamide

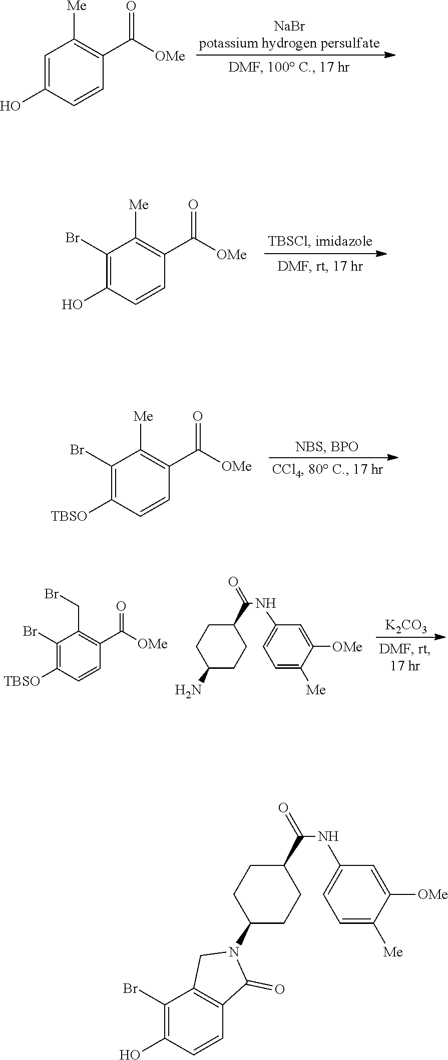

### Step 1

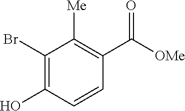 [SMILES: COC(=O)c1ccc(O)c(Br)c1C]

### Methyl 3-bromo-4-hydroxy-2-methylbenzoate

To a solution of methyl 4-hydroxy-2-methyl-benzoate (1.00 g, 6.02 mmol) in DMF (10 mL) was added potassium hydrogen persulfate (4.06 g, 6.62 mmol) and sodium bromide (619 mg, 6.02 mmol). The mixture was stirred at 100° C. for 17 hours. The mixture was poured into EtOAc (50 mL) and washed with water (30 mL*3). The organic layer was dried and concentrated. The residue was purified by prep-HPLC to give methyl 3-bromo-4-hydroxy-2-methylbenzoate (500 mg, 2.04 mmol, 33.9% yield) was obtained as yellow solid. LCMS ESI m/z 247.0 [M+H]⁺.

### Step 2

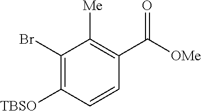 [SMILES: COC(=O)c1ccc(O[Si](C)(C)C(C)(C)C)c(Br)c1C]

### Methyl 3-bromo-4-(tert-butyldimethylsilyloxy)-2-methylbenzoate

A solution of methyl 3-bromo-4-hydroxy-2-methyl-benzoate (300 mg, 1.22 mmol), TBSCl (131 mg, 1.59 mmol) and imidazole (125 mg, 1.84 mmol) in DMF (5 mL) was stirred at 25° C. for 17 hours. The mixture was worked up with EtOAc and H₂O. The organic phase was concentrated in vacuum. The residue was purified by silica column (PE:EtOAc=30:1 to 1:1) to give methyl 3-bromo-4-(tert-butyldimethylsilyloxy)-2-methylbenzoate (100 mg, 278 μmol, 22.7% yield) as colorless oil. ¹H NMR (400 MHz, DMSO-d₆) δ 7.72 (d, J=8.0 Hz, 1H), 6.95 (d, J=8.0 Hz, 1H), 3.80 (s, 3H), 2.59 (s, 3H), 1.01 (s, 9H), 0.27 (s, 6H).

### Step 3

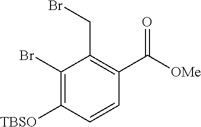 [SMILES: COC(=O)c1ccc(O[Si](C)(C)C(C)(C)C)c(Br)c1CBr]

### Methyl 3-bromo-2-(bromomethyl)-4-(tert-butyldimethylsilyloxy)benzoate

To a solution of methyl 3-bromo-4-[tert-butyl(dimethyl)silyl]oxy-2-methyl-benzoate (370 mg, 1.03 mmol) in CCl₄ (5.0 mL) was added 1-bromopyrrolidine-2,5-dione (220 mg, 1.24 mmol) and benzoyl benzenecarboperoxoate (24.9 mg, 103 μmol). The mixture was stirred at 80° C. for 17 hours. The mixture was filtered and concentrated to give the crude product methyl 3-bromo-2-(bromomethyl)-4-[tert-butyl(dimethyl)silyl]oxy-benzoate (200 mg, 456 μmol, 44.3% yield) as colorless oil.

### Step 4

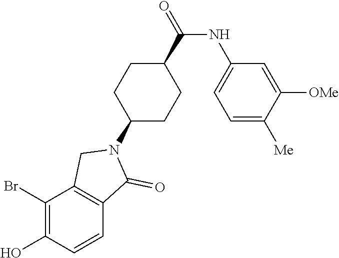 [SMILES: COc1cc(NC(=O)[C@H]2CC[C@@H](N3Cc4c(ccc(O)c4Br)C3=O)CC2)ccc1C]

### (1s,4s)-4-(4-Bromo-5-hydroxy-1-oxoisoindolin-2-yl)-N-(3-methoxy-4-methylphenyl)cyclohexanecarboxamide

To a solution of methyl 3-bromo-2-(bromomethyl)-4-[tert-butyl(dimethyl)silyl]oxy-benzoate (200 mg, 456.39 μmol) and 4-amino-N-(3-methoxy-4-methyl-phenyl)cyclohexanecarboxamide (120 mg, 456 μmol) in DMF (5 mL) was added K₂CO₃ (630 mg, 4.56 mmol). The mixture was stirred at 25° C. for 17 hours. The mixture was filtered and the filtrate was concentrated. The residue was purified by prep-HPLC to give (1s,4s)-4-(4-bromo-5-hydroxy-1-oxoisoindolin-2-yl)-N-(3-methoxy-4-methylphenyl)cyclohexanecarboxamide (60 mg, 130 μmol, 27% yield) as a white solid. LCMS ESI m/z 472.6 [M+H]⁺. ¹HNMR 1H NMR (400 MHz, DMSO-d₆) δ 9.71 (s, 1H), 7.48 (d, J=8.2 Hz, 1H), 7.32 (d, J=1.6 Hz, 1H), 7.10 (d, J=1.8 Hz, 1H), 7.02 (dd, J=8.1, 5.6 Hz, 2H), 4.27 (s, 2H), 4.07-4.02 (m, 1H), 3.76 (s, 3H), 2.67-2.65 (m, 1H), 2.16-1.90 (m, 7H), 1.72-1.58 (m, 4H).

### Example 190

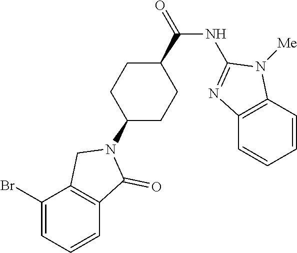 [SMILES: Cn1c(NC(=O)[C@H]2CC[C@@H](N3Cc4c(Br)cccc4C3=O)CC2)nc2ccccc21]

### (1s,4s)-4-(4-Bromo-1-oxoisoindolin-2-yl)-N-(1-methyl-1H-benzo[d]imidazol-2-yl)cyclohexanecarboxamide

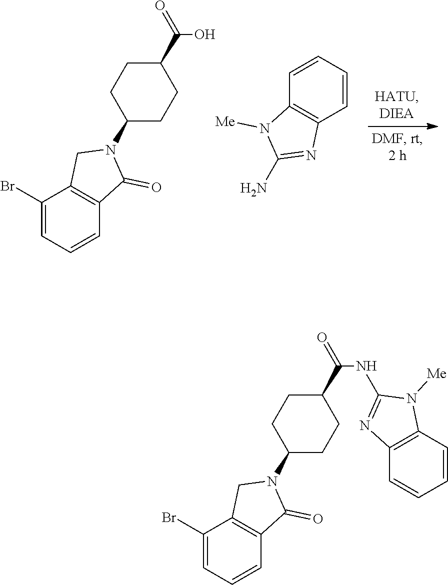 [SMILES: O=C1c2cccc(Br)c2CN1[C@H]1CC[C@@H](C(=O)O)CC1 ; Cn1c(N)nc2ccccc21 ; Cn1c(NC(=O)[C@H]2CC[C@@H](N3Cc4c(Br)cccc4C3=O)CC2)nc2ccccc21]

### Step 1

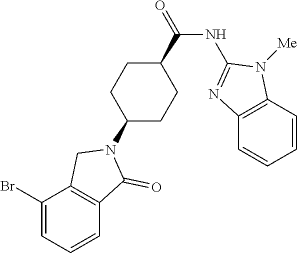 [SMILES: Cn1c(NC(=O)[C@H]2CC[C@@H](N3Cc4c(Br)cccc4C3=O)CC2)nc2ccccc21]

### (1s,4s)-4-(4-Bromo-1-oxoisoindolin-2-yl)-N-(1-methyl-1H-benzo[d]imidazol-2-yl)cyclohexanecarboxamide

Following the synthetic procedure for Example 185, but starting with 1-methyl-1H-benzo[d]imidazol-2-amine gave the title compound as a white solid. LCMS (ESI) m/z: 468.7 [M+H]⁺. ¹H NMR (400 MHz, DMSO-d₆) δ 7.79 (d, J=8.0 Hz, 1H), 7.70 (d, J=7.2 Hz, 1H), 7.59-7.35 (m, 3H), 7.28-7.12 (m, 2H), 4.40-4.25 (m, 2H), 4.20-4.02 (m, 1H), 3.70-3.50 (m, 3H), 2.99-2.75 (m, 1H), 2.72-2.57 (m, 1H), 2.4-2.1 (m, 2H), 2.05-1.85 (m, 2H), 1.85-1.55 (m, 4H).

### Example 191

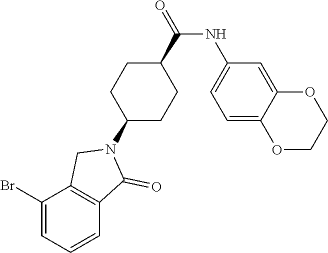 [SMILES: O=C1c2cccc(Br)c2CN1[C@H]1CC[C@@H](C(=O)Nc2ccc3c(c2)OCCO3)CC1]

### (1s,4s)-4-(4-Bromo-1-oxoisoindolin-2-yl)-N-(2,3-dihydrobenzo[b][1,4]dioxin-6-yl)cyclohexanecarboxamide

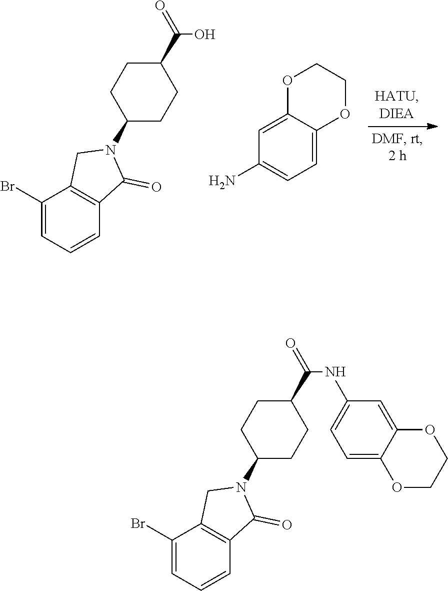 [SMILES: O=C1c2cccc(Br)c2CN1[C@H]1CC[C@@H](C(=O)O)CC1 ; Nc1ccc2c(c1)OCCO2 ; O=C1c2cccc(Br)c2CN1[C@H]1CC[C@@H](C(=O)Nc2ccc3c(c2)OCCO3)CC1]

### Step 1

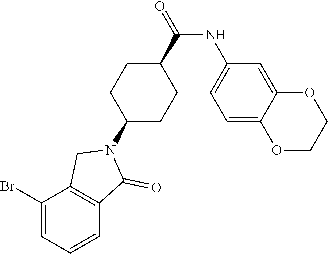 [SMILES: O=C1c2cccc(Br)c2CN1[C@H]1CC[C@@H](C(=O)Nc2ccc3c(c2)OCCO3)CC1]

### (1s,4s)-4-(4-Bromo-1-oxoisoindolin-2-yl)-N-(2,3-dihydrobenzo[b][1,4]dioxin-6-yl)cyclohexanecarboxamide

Following the synthetic procedure for Example 185, but starting with 2,3-dihydrobenzo[b][1,4]dioxin-6-amine gave the title compound as a white solid. LCMS (ESI) m/z: 470.7 [M+H]⁺. ¹H NMR (400 MHz, DMSO-d₆) δ 9.61 (s, 1H), 7.80 (dd, J=8.0, 0.8 Hz, 1H), 7.74-7.64 (m, 1H), 7.46 (t, J=7.6 Hz, 1H), 7.26 (d, J=2.4 Hz, 1H), 7.01 (dd, J=8.8, 2.4 Hz, 1H), 6.76 (d, J=8.8 Hz, 1H), 4.36 (s, 2H), 4.30-4.15 (m, 4H), 4.13-4.00 (m, 1H), 2.64-2.62 (m, 1H), 2.18-1.9 (m, 4H), 1.81-1.57 (m, 4H).

### Example 192

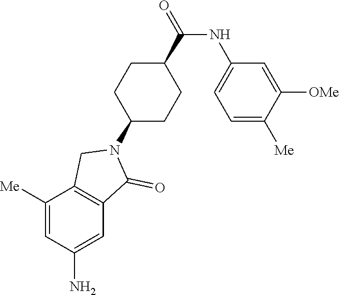 [SMILES: COc1cc(NC(=O)[C@H]2CC[C@@H](N3Cc4c(C)cc(N)cc4C3=O)CC2)ccc1C]

### (1s,4s)-4-(6-Amino-4-methyl-1-oxoisoindolin-2-yl)-N-(3-methoxy-4-methylphenyl)cyclohexanecarboxamide

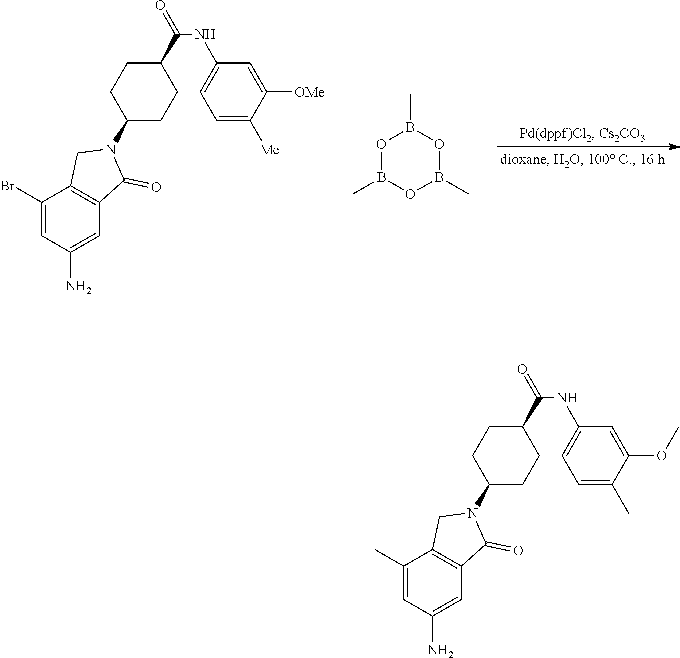

### Step 1

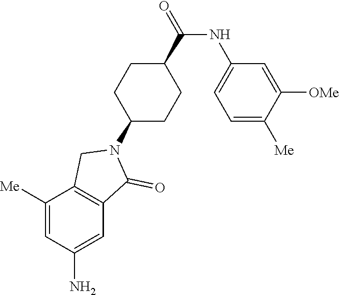 [SMILES: COc1cc(NC(=O)[C@H]2CC[C@@H](N3Cc4c(C)cc(N)cc4C3=O)CC2)ccc1C]

### (1s,4s)-4-(6-Amino-4-methyl-1-oxoisoindolin-2-yl)-N-(3-methoxy-4-methylphenyl)cyclohexanecarboxamide

A mixture of 4-(6-amino-4-bromo-1-oxo-isoindolin-2-yl)-N-(3-methoxy-4-methyl-phenyl)cyclohexanecarboxamide (50 mg, 110 μmol), 2,4,6-trimethyl-1,3,5,2,4,6-trioxatriborinane (26.6 mg, 212 μmol), Pd(dppf)Cl₂ (7.7 mg, 11 μmol) and Cs₂CO₃ (104 mg, 318 μmol) in dioxane (1.0 mL)/H₂O (0.5 mL) was stirred at 100° C. for 16 hours under N₂. The mixture was poured into water and extracted with EtOAc. The organic extracts were washed with brine, dried by Na₂SO₄, filtered and concentrated to give a crude oil. The crude product was purified by silica gel chromatography to give crude product (25 mg) as a yellow solid. The solid was purified by prep-HPLC to give (1s,4s)-4-(6-amino-4-methyl-1-oxoisoindolin-2-yl)-N-(3-methoxy-4-methylphenyl)cyclohexanecarboxamide (5.0 mg, 12 μmol, 11% yield) as a white solid. LCMS ESI m/z: 408.3 [M+H]⁺. ¹H NMR (400 MHz, DMSO-d₆) δ 9.69 (s, 1H), 7.32 (d, J=1.7 Hz, 1H), 7.12 (dd, J=8.0, 1.8 Hz, 1H), 7.02 (d, J=8.5 Hz, 1H), 6.65 (d, J=1.8 Hz, 1H), 6.57 (d, J=1.2 Hz, 1H), 5.16 (s, 2H), 4.18 (s, 2H), 4.11-4.01 (m, 1H), 3.76 (s, 3H), 2.68-2.66 (m, 1H), 2.18 (s, 3H), 2.13-2.06 (m, 5H), 1.99-1.94 (m, 2H), 1.75-1.67 (m, 2H), 1.62-1.52 (m, 2H).

### Example 193

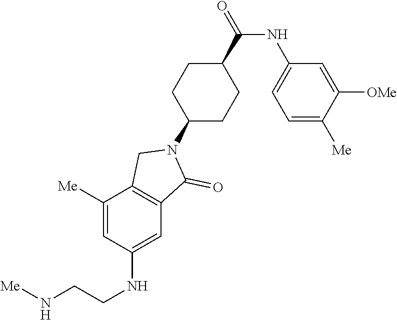 [SMILES: CNCCNc1cc(C)c2c(c1)C(=O)N([C@H]1CC[C@@H](C(=O)Nc3ccc(C)c(OC)c3)CC1)C2]

### (1s,4s)-N-(3-methoxy-4-methylphenyl)-4-(4-methyl-6-(2-(methylamino)ethylamino)-1-oxoisoindolin-2-yl)cyclohexanecarboxamide

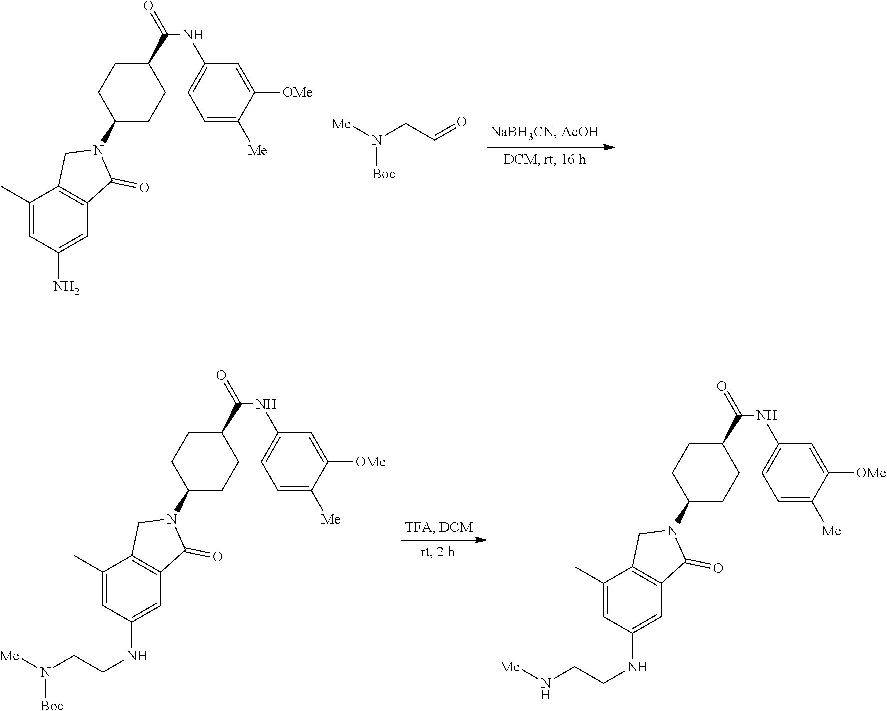 [SMILES: COc1cc(NC(=O)[C@H]2CC[C@@H](N3Cc4c(C)cc(N)cc4C3=O)CC2)ccc1C ; CN(CC=O)C(=O)OC(C)(C)C ; COc1cc(NC(=O)[C@H]2CC[C@@H](N3Cc4c(C)cc(NCCN(C)C(=O)OC(C)(C)C)cc4C3=O)CC2)ccc1C ; CNCCNc1cc(C)c2c(c1)C(=O)N([C@H]1CC[C@@H](C(=O)Nc3ccc(C)c(OC)c3)CC1)C2]

### Step 1 and 2

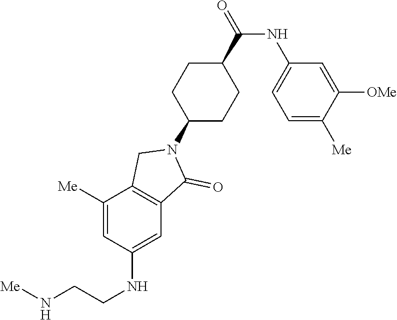 [SMILES: CNCCNc1cc(C)c2c(c1)C(=O)N([C@H]1CC[C@@H](C(=O)Nc3ccc(C)c(OC)c3)CC1)C2]

### (1s,4s)-N-(3-Methoxy-4-methylphenyl)-4-(4-methyl-6-(2-(methylamino)ethylamino)-1-oxoisoindolin-2-yl)cyclohexanecarboxamide

Following the synthetic procedure for Example 175, but starting with (1s,4s)-4-(6-amino-4-methyl-1-oxoisoindolin-2-yl)-N-(3-methoxy-4-methylphenyl)cyclohexanecarboxamide gave the title compound as a white solid. LCMS (ESI) m/z: 464.9 [M+H]⁺. ¹H NMR (400 MHz, DMSO-d₆) δ 9.70 (s, 1H), 7.32 (d, J=1.6 Hz, 1H), 7.12 (dd, J=8.0, 2.0 Hz, 1H), 7.02 (d, J=8.4 Hz, 1H), 6.65-6.55 (m, 2H), 5.68-5.63 (m, 1H), 4.20 (s, 2H), 4.15-4.00 (m, 1H), 3.76 (s, 3H), 3.44-3.40 (m, 1H), 3.40-3.33 (m, 3H), 3.24-3.16 (m, 1H), 3.15-3.07 (m, 2H), 2.83 (s, 1H), 2.70-2.63 (m, 2H), 2.30 (s, 2H), 2.20 (s, 3H), 2.15-2.05 (m, 5H), 2.02-1.85 (m, 2H), 1.77-1.65 (m, 2H), 1.63-1.50 (m, 2H).

### Example 194

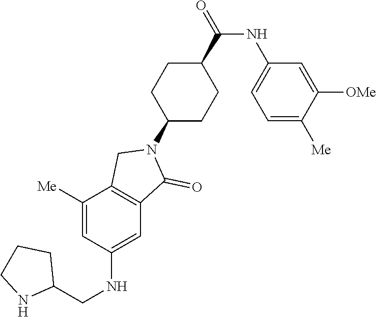 [SMILES: COc1cc(NC(=O)[C@H]2CC[C@@H](N3Cc4c(C)cc(NCC5CCCN5)cc4C3=O)CC2)ccc1C]

### (1s,4s)-N-(3-methoxy-4-methylphenyl)-4-(4-methyl-1-oxo-6-(pyrrolidin-2-ylmethylamino) isoindolin-2-yl)cyclohexanecarboxamide

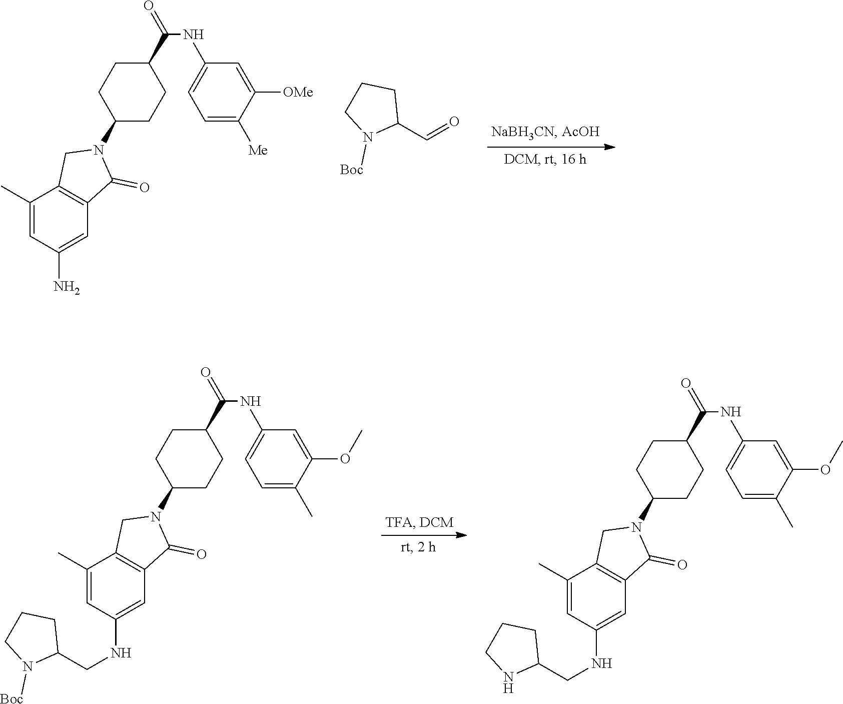 [SMILES: COc1cc(NC(=O)[C@H]2CC[C@@H](N3Cc4c(C)cc(N)cc4C3=O)CC2)ccc1C ; CC(C)(C)OC(=O)N1CCCC1C=O ; COc1cc(NC(=O)[C@H]2CC[C@@H](N3Cc4c(C)cc(NCC5CCCN5C(=O)OC(C)(C)C)cc4C3=O)CC2)ccc1C ; COc1cc(NC(=O)[C@H]2CC[C@@H](N3Cc4c(C)cc(NCC5CCCN5)cc4C3=O)CC2)ccc1C]

### Step 1 and 2

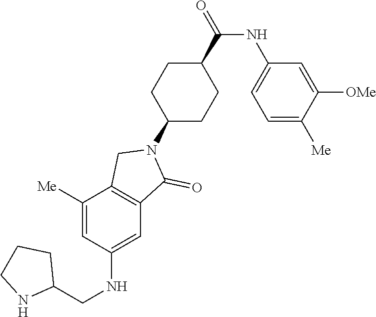 [SMILES: COc1cc(NC(=O)[C@H]2CC[C@@H](N3Cc4c(C)cc(NCC5CCCN5)cc4C3=O)CC2)ccc1C]

### (1s,4s)-N-(3-methoxy-4-methylphenyl)-4-(4-methyl-1-oxo-6-(pyrrolidin-2-ylmethylamino) isoindolin-2-yl)cyclohexanecarboxamide

Following the synthetic procedure for Example 193, but starting with tert-butyl 2-formylpyrrolidine-1-carboxylate gave the title compound as a white solid. LCMS (ESI) m/z: 490.9 [M+H]⁺. ¹H NMR (400 MHz, MeOD-d₄) δ 7.29 (d, J=1.8 Hz, 1H), 7.03 (d, J=8.2 Hz, 1H), 6.96 (dd, J=8.0, 1.9 Hz, 1H), 6.82 (d, J=2.0 Hz, 1H), 6.73 (d, J=1.3 Hz, 1H), 4.34 (s, 2H), 4.28-4.18 (m, 1H), 3.83 (s, 3H), 3.46-3.40 (m, 1H), 3.26-3.15 (m, 2H), 3.08-3.01 (m, 1H), 2.97-2.93 (m, 1H), 2.74-2.72 (m, 1H), 2.28 (s, 3H), 2.23-2.17 (m, 2H), 2.16-2.07 (m, 5H), 2.05-2.00 (m, 1H), 1.92-1.79 (m, 4H), 1.78-1.71 (m, 2H), 1.59-1.54 (m, 1H).

### Example 195

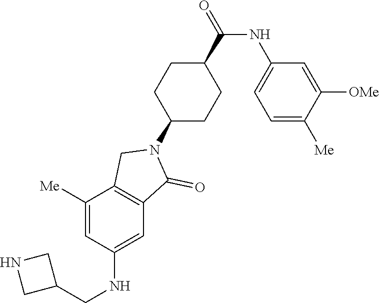 [SMILES: COc1cc(NC(=O)[C@H]2CC[C@@H](N3Cc4c(C)cc(NCC5CNC5)cc4C3=O)CC2)ccc1C]

### (1s,4s)-4-(6-(Azetidin-3-ylmethylamino)-4-methyl-1-oxoisoindolin-2-yl)-N-(3-methoxy-4-methylphenyl)cyclohexanecarboxamide

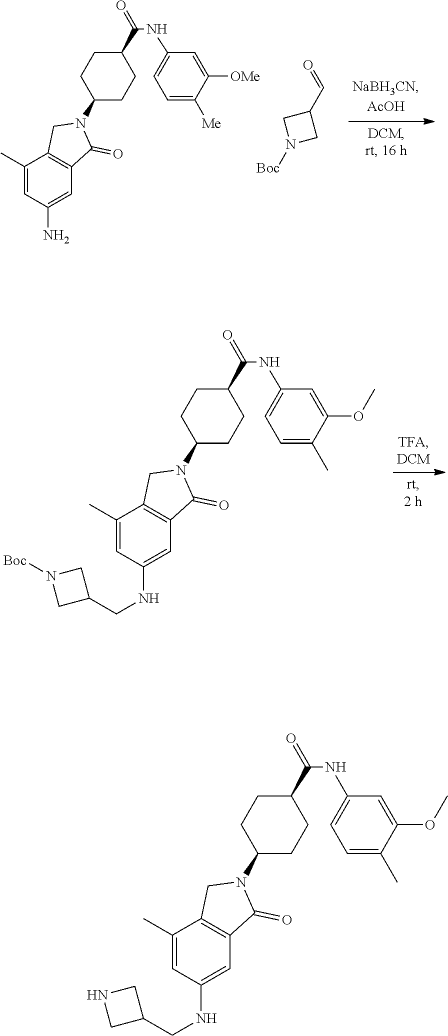 [SMILES: COc1cc(NC(=O)[C@H]2CC[C@@H](N3Cc4c(C)cc(N)cc4C3=O)CC2)ccc1C ; CC(C)(C)OC(=O)N1CC(C=O)C1 ; COc1cc(NC(=O)[C@H]2CC[C@@H](N3Cc4c(C)cc(NCC5CN(C(=O)OC(C)(C)C)C5)cc4C3=O)CC2)ccc1C ; COc1cc(NC(=O)[C@H]2CC[C@@H](N3Cc4c(C)cc(NCC5CNC5)cc4C3=O)CC2)ccc1C]

### Step 1 and 2

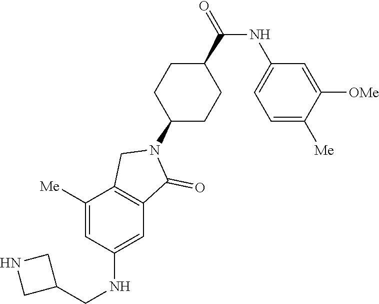 [SMILES: COc1cc(NC(=O)[C@H]2CC[C@@H](N3Cc4c(C)cc(NCC5CNC5)cc4C3=O)CC2)ccc1C]

### (1s,4s)-4-(6-(Azetidin-3-ylmethylamino)-4-methyl-1-oxoisoindolin-2-yl)-N-(3-methoxy-4-methylphenyl)cyclohexanecarboxamide

Following the synthetic procedure for Example 193, but starting with tert-butyl 3-formylazetidine-1-carboxylate gave the title compound as a white solid. LCMS (ESI) m/z: 476.9 [M+H]⁺. ¹H NMR (400 MHz, MeOD-d₄) δ 7.29 (d, J=1.7 Hz, 1H), 7.03 (d, J=8.1 Hz, 1H), 6.96 (dd, J=8.0, 1.9 Hz, 1H), 6.79 (d, J=1.9 Hz, 1H), 6.70 (d, J=1.3 Hz, 1H), 4.34 (s, 2H), 4.26-4.20 (m, 1H), 3.90-3.76 (m, 5H), 3.59-3.55 (m, 2H), 3.37-2.33 (m, 2H), 3.14-3.08 (m, 1H), 2.74-2.72 (m, 1H), 2.27 (s, 3H), 2.23-2.19 (m 2H), 2.16-2.04 (m, 5H), 1.87-1.82 (m, 2H), 1.79-1.72 (m, 2H).

### Example 196

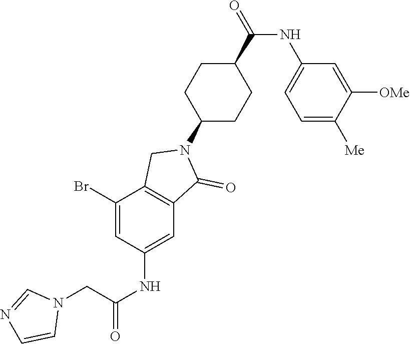 [SMILES: COc1cc(NC(=O)[C@H]2CC[C@@H](N3Cc4c(Br)cc(NC(=O)Cn5ccnc5)cc4C3=O)CC2)ccc1C]

### (1s,4s)-4-(6-(2-(1H-Imidazol-1-yl) acetamido)-4-bromo-1-oxoisoindolin-2-yl)-N-(3-methoxy-4-methylphenyl)cyclohexanecarboxamide

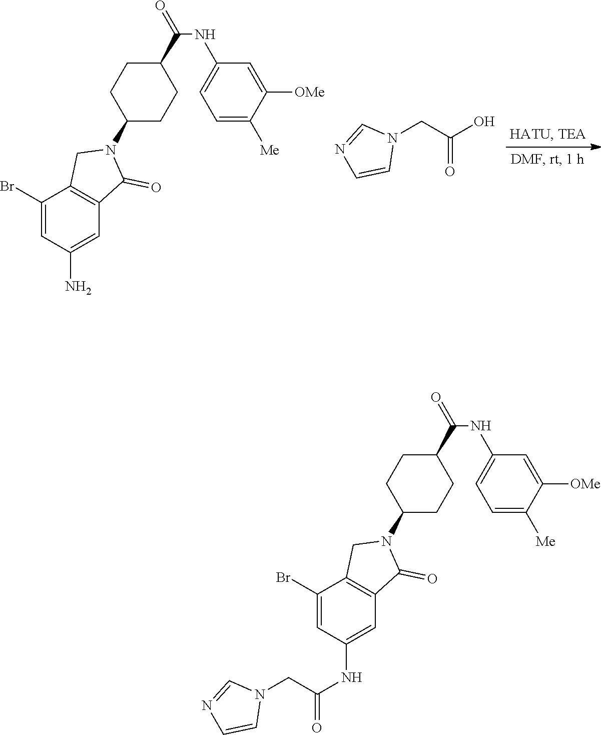 [SMILES: COc1cc(NC(=O)[C@H]2CC[C@@H](N3Cc4c(Br)cc(N)cc4C3=O)CC2)ccc1C ; O=C(O)Cn1ccnc1 ; COc1cc(NC(=O)[C@H]2CC[C@@H](N3Cc4c(Br)cc(NC(=O)Cn5ccnc5)cc4C3=O)CC2)ccc1C]

### Step 1

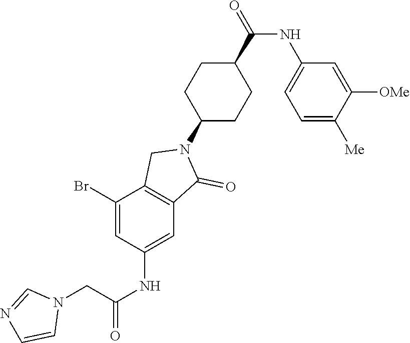 [SMILES: COc1cc(NC(=O)[C@H]2CC[C@@H](N3Cc4c(Br)cc(NC(=O)Cn5ccnc5)cc4C3=O)CC2)ccc1C]

### (1s,4s)-4-(6-(2-(1H-Imidazol-1-yl) acetamido)-4-bromo-1-oxoisoindolin-2-yl)-N-(3-methoxy-4-methylphenyl)cyclohexanecarboxamide

A solution of 2-imidazol-1-ylacetic acid (90 mg, 710 μmol), N,N-diethylethanamine (217 mg, 2.14 mmol, 298 μL) and [dimethylamino (triazolo[4,5-b]pyridin-3-yloxy)methylene]-dimethyl-ammonium; hexafluorophosphate (814 mg, 2.14 mmol) in DMF (1.8 mL) was stirred for 10 minutes, 4-(6-amino-4-bromo-1-oxo-isoindolin-2-yl)-N-(3-methoxy-4-methyl-phenyl)cyclohexanecarboxamide (100 mg, 210 μmol) was added. The resulting mixture was stirred for 1 hour at 25° C. To the reaction mixture was added EtOAc (50 mL) and washed with H₂O (3×50 mL). The combined organic extracts were dried over Na₂SO₄ and evaporated in vacuo. The residue was purified by Prep-HPLC to get (1s,4s)-4-(6-(2-(1H-imidazol-1-yl) acetamido)-4-bromo-1-oxoisoindolin-2-yl)-N-(3-methoxy-4-methylphenyl)cyclohexanecarboxamide (0.0070 g, 12 μmol, 1.7% yield) as a white solid. LCMS ESI m/z: 579.7 [M+H]⁺. ¹H NMR (400 MHz, MeOD-d₄) δ 8.73 (s, 1H), 8.06 (s, 1H), 7.94 (d, J=1.6 Hz, 1H), 7.56 (s, 1H), 7.49 (s, 1H), 7.28 (d, J=2.0 Hz, 1H), 7.03 (d, J=8.4 Hz, 1H), 6.95 (dd, J=8.0, 2.0 Hz, 1H), 5.19 (s, 2H), 4.44 (s, 2H), 4.30-4.20 (m, 1H), 3.83 (s, 3H), 2.74-2.72 (m, 1H), 2.31-2.03 (m, 7H), 1.92-1.70 (m, 4H).

### Example 197

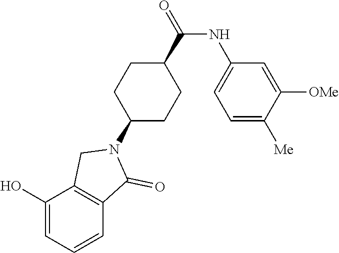 [SMILES: COc1cc(NC(=O)[C@H]2CC[C@@H](N3Cc4c(O)cccc4C3=O)CC2)ccc1C]

### (1s,4s)-4-(4-Hydroxy-1-oxoisoindolin-2-yl)-N-(3-methoxy-4-methylphenyl)cyclohexanecarboxamide

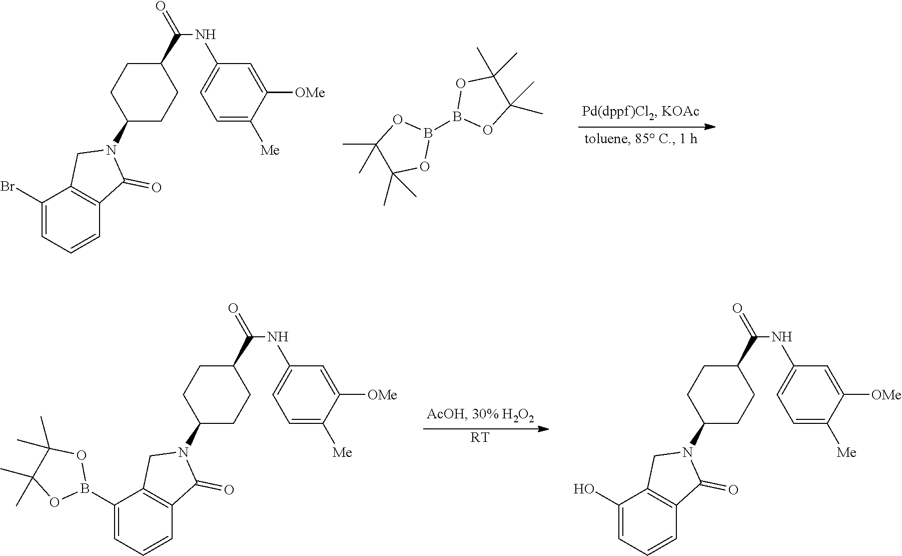

### Step 1

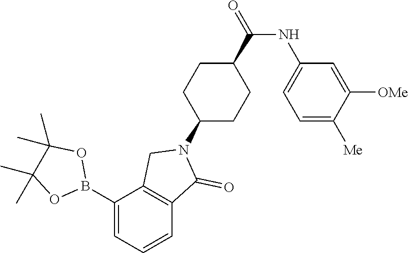 [SMILES: COc1cc(NC(=O)[C@H]2CC[C@@H](N3Cc4c(B5OC(C)(C)C(C)(C)O5)cccc4C3=O)CC2)ccc1C]

### (1s,4s)-N-(3-Methoxy-4-methylphenyl)-4-(1-oxo-4-(4,4,5,5-tetramethyl-1,3,2-dioxaborolan-2-yl)isoindolin-2-yl)cyclohexanecarboxamide

A mixture of (1s,4s)-4-(4-bromo-1-oxoisoindolin-2-yl)-N-(3-methoxy-4-methylphenyl)cyclohexanecarboxamide (125 mg, 273 μmol), 4,4,5,5-tetramethyl-2-(4,4,5,5-tetramethyl-1,3,2-dioxaborolan-2-yl)-1,3,2-dioxaborolane (83.3 mg, 328 μmol), Pd(dppf)Cl₂ (20.0 mg, 27.3 μmol) and KOAc (54 mg, 550 μmol, 34.17 μL) in toluene (4 mL) was stirred at 85° C. for 1 hour. The reaction mixture was filtered and concentrated. The residue was purified by Prep-HPLC to give (1s,4s)-N-(3-methoxy-4-methylphenyl)-4-(1-oxo-4-(4,4,5,5-tetramethyl-1,3,2-dioxaborolan-2-yl)isoindolin-2-yl)cyclohexanecarboxamide (80 mg, 160 μmol, 58% yield) as yellow oil. LCMS (ESI) m/z: 504.9 [M+H]⁺.

### Step 2

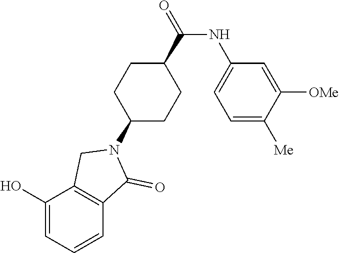 [SMILES: COc1cc(NC(=O)[C@H]2CC[C@@H](N3Cc4c(O)cccc4C3=O)CC2)ccc1C]

### (1s,4s)-4-(4-Hydroxy-1-oxoisoindolin-2-yl)-N-(3-methoxy-4-methylphenyl)cyclohexanecarboxamide

To a solution of (1s,4s)-N-(3-methoxy-4-methylphenyl)-4-(1-oxo-4-(4,4,5,5-tetramethyl-1,3,2-dioxaborolan-2-yl)isoindolin-2-yl)cyclohexanecarboxamide (50 mg, 99 μmol) in AcOH (1 mL) was added hydrogen peroxide (150 mg, 1.4 mmol, 140 μL, 30% wt.) at 0° C. The reaction was gradually warmed to RT and stirred for 70 minutes. The reaction mixture was extracted with EtOAc, washed with water, the organic layer was concentrated to the crude and purified by Prep-HPLC to give (1s,4s)-4-(4-hydroxy-1-oxoisoindolin-2-yl)-N-(3-methoxy-4-methylphenyl)cyclohexanecarboxamide (11 mg, 28 μmol, 28% yield) as a white solid. LCMS (ESI) m/z: 394.9 [M+H]⁺. ¹H NMR (400 MHz, DMSO-d₆) δ 10.03 (b, 1H), 9.72 (s, 1H), 7.34 (d, J=1.7 Hz, 1H), 7.29 (t, J=7.7 Hz, 1H), 7.15-7.07 (m, 2H), 7.02 (d, J=8.2 Hz, 1H), 6.95 (d, J=7.9 Hz, 1H), 4.31 (s, 2H), 4.18-4.03 (m, 1H), 3.76 (s, 3H), 2.68-2.66 (m, 1H), 2.12-2.10 (m, 2H), 2.05-1.92 (m, 2H), 1.79-1.56 (m, 4H).

### Example 198

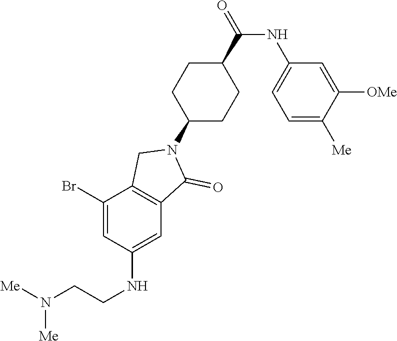 [SMILES: COc1cc(NC(=O)[C@H]2CC[C@@H](N3Cc4c(Br)cc(NCCN(C)C)cc4C3=O)CC2)ccc1C]

### (1s,4s)-4-(4-Bromo-6-(2-(dimethylamino)ethylamino)-1-oxoisoindolin-2-yl)-N-(3-methoxy-4-methylphenyl)cyclohexanecarboxamide

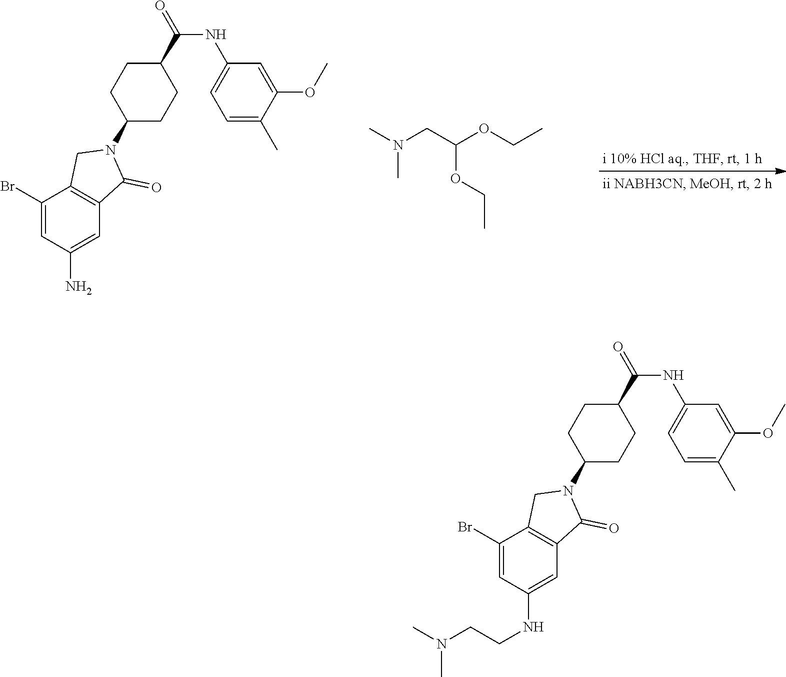

### Step 1

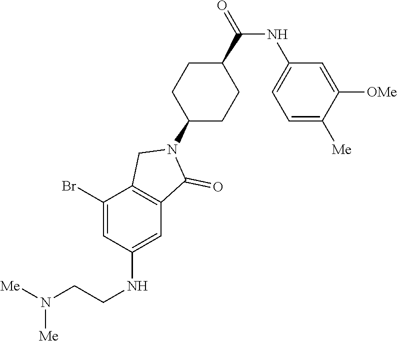 [SMILES: COc1cc(NC(=O)[C@H]2CC[C@@H](N3Cc4c(Br)cc(NCCN(C)C)cc4C3=O)CC2)ccc1C]

### (1s,4s)-4-(4-Bromo-6-(2-(dimethylamino)ethylamino)-1-oxoisoindolin-2-yl)-N-(3-methoxy-4-methylphenyl)cyclohexanecarboxamide

To a solution of 2,2-diethoxy-N,N-dimethyl-ethanamine (75 mg, 470 μmol, 86 μL) in THF (10 mL) was added 10% HCl aq. (8.0 mL, 100 μmol). The mixture was stirred at 25° C. for 60 minutes. The reaction mixture was concentrated to give a crude oil. Then the oil diluted in MeOH (10 mL), 4-(6-amino-4-bromo-1-oxo-isoindolin-2-yl)-N-(3-methoxy-4-methyl-phenyl)cyclohexanecarboxamide (200 mg, 420 μmol) and sodium cyanoborohydride (133 mg, 2.12 mmol) was added. The mixture was stirred at 25° C. for 2 hours. The reaction solution was mixed with saturated sodium carbonate aqueous solution (20 ml), extracted with EtOAc (30 mL*3). The organic extracts were washed with brine, dried by Na₂SO₄, filtered and concentrated to give a crude oil. The crude product was purified by silica gel chromatography to give a yellow solid (80 mg). The yellow solid was purified by prep-HPLC to give (1s,4s)-4-(4-bromo-6-(2-(dimethylamino)ethylamino)-1-oxoisoindolin-2-yl)-N-(3-methoxy-4-methylphenyl)cyclohexanecarboxamide (20 mg, 36 μmol, 8.6% yield) as a white solid. LCMS ESI m/z: 542.8 [M+H]⁺. ¹H NMR (400 MHz, DMSO-d₆) δ 9.70 (s, 1H), 7.32 (d, J=1.7 Hz, 1H), 7.11 (dd, J=8.0, 1.8 Hz, 1H), 7.04-7.00 (m, 2H), 6.81 (d, J=1.9 Hz, 1H), 6.00 (t, J=5.4 Hz, 1H), 4.18 (s, 2H), 4.11-4.03 (m, 1H), 3.76 (s, 3H), 3.12 (dd, J=12.0, 6.4 Hz, 2H), 2.67-2.65 (m, 1H), 2.42 (t, J=6.5 Hz, 2H), 2.18 (s, 6H), 2.11-2.06 (m, 5H), 2.03-1.92 (m, 2H), 1.74-1.69 (m, 2H), 1.65-1.57 (m, 2H).

### Example 199

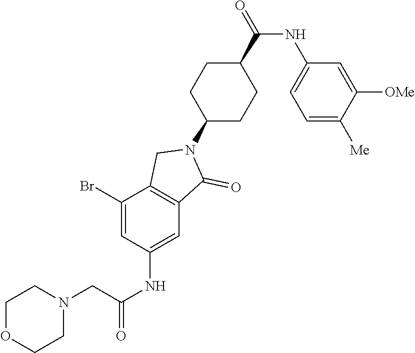 [SMILES: COc1cc(NC(=O)[C@H]2CC[C@@H](N3Cc4c(Br)cc(NC(=O)CN5CCOCC5)cc4C3=O)CC2)ccc1C]

### (1s,4s)-4-(4-Bromo-6-(2-morpholinoacetamido)-1-oxoisoindolin-2-yl)-N-(3-methoxy-4-methylphenyl)cyclohexanecarboxamide

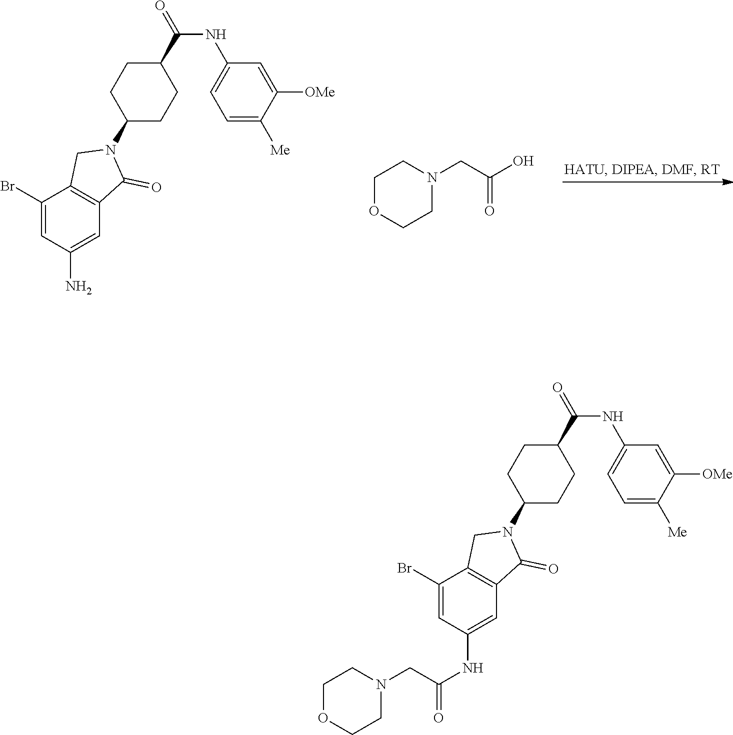 [SMILES: COc1cc(NC(=O)[C@H]2CC[C@@H](N3Cc4c(Br)cc(N)cc4C3=O)CC2)ccc1C ; O=C(O)CN1CCOCC1 ; COc1cc(NC(=O)[C@H]2CC[C@@H](N3Cc4c(Br)cc(NC(=O)CN5CCOCC5)cc4C3=O)CC2)ccc1C]

### Step 1

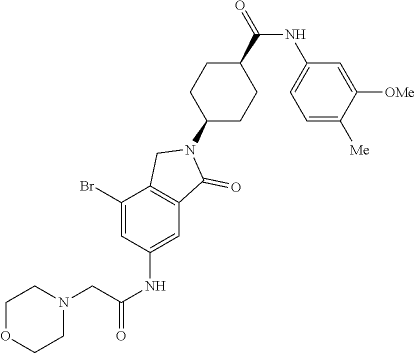 [SMILES: COc1cc(NC(=O)[C@H]2CC[C@@H](N3Cc4c(Br)cc(NC(=O)CN5CCOCC5)cc4C3=O)CC2)ccc1C]

### (1s,4s)-4-(4-Bromo-6-(2-morpholinoacetamido)-1-oxoisoindolin-2-yl)-N-(3-methoxy-4-methylphenyl)cyclohexanecarboxamide

Following the synthetic procedure for Example 196, but starting with 2-morpholinoacetic acid gave the title compound as a white solid. LCMS (ESI) m/z: 599.8 [M+H]⁺. ¹H NMR (400 MHz, DMSO-d₆) 010.07 (s, 1H), 9.71 (s, 1H), 8.13 (d, J=1.5 Hz, 1H), 8.05 (d, J=1.4 Hz, 1H), 7.32 (s, 1H), 7.12 (dd, J=8.0, 1.5 Hz, 1H), 7.02 (d, J=8.1 Hz, 1H), 4.33 (s, 2H), 4.12-4.08 (m, 1H), 3.76 (s, 3H), 3.69-3.61 (m, 4H), 3.16 (s, 2H), 2.68-2.66 (m, 1H), 2.52-2.50 (m, 4H), 2.16-1.94 (m, 7H), 1.78-1.58 (m, 4H).

### Example 200

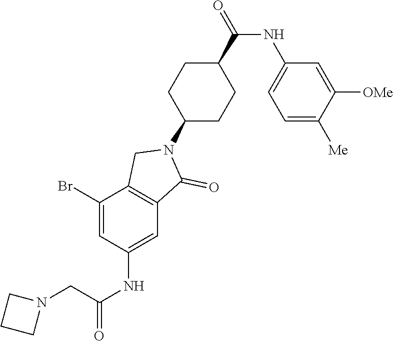 [SMILES: COc1cc(NC(=O)[C@H]2CC[C@@H](N3Cc4c(Br)cc(NC(=O)CN5CCC5)cc4C3=O)CC2)ccc1C]

### (1s,4s)-4-(6-(2-(Azetidin-1-yl) acetamido)-4-bromo-1-oxoisoindolin-2-yl)-N-(3-methoxy-4-methylphenyl)cyclohexanecarboxamide

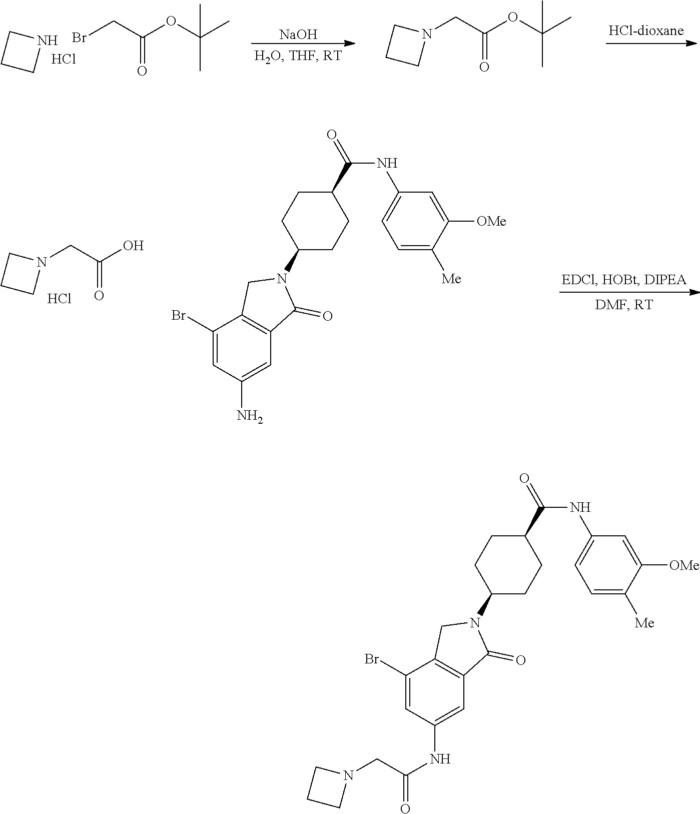

### Step 1

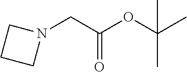

### tert-Butyl 2-(azetidin-1-yl)acetate

To a solution of azetidine (500 mg, 5.34 mmol, hydrochloride salt) and tert-butyl 2-bromoacetate (520 mg, 2.7 mmol, 390 μL) in H₂O (3 mL) and THF (10 mL) was added NaOH (210 mg, 5.3 mmol) at 25° C. with stirring for 3 hours. The reaction mixture was added with water and ethyl acetate, and the organic layer was separated. The aqueous layer was extracted three times with ethyl acetate, the combined organic layer was washed three times with saturated brine, and the solvent was evaporated under reduced pressure to obtain tert-butyl 2-(azetidin-1-yl)acetate (800 mg, 87% yield) as a yellow oil. ¹H NMR (400 MHz, CDCl₃) δ 3.24 (t, J=7.0 Hz, 4H), 3.05 (s, 2H), 2.08-2.00 (m, 2H), 1.38 (s, 9H).

### Step 2

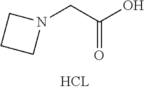

### 2-(Azetidin-1-yl)acetic acid hydrochloride

Tert-Butyl 2-(azetidin-1-yl)acetate (350 mg) prepared in Step 1 above was added with 4 N hydrochloric acid solution in 1,4-dioxane (5 ml). The mixture was stirred at room temperature for 20 hours. The deposited solids were collected by filtration, washed with diethyl ether, and then dried under reduced pressure to obtain 2-(azetidin-1-yl) acetic acid hydrochloride (300 mg) as a white solid without further purification. ¹H NMR (400 MHz, CDCl₃) δ 13.68 (b, 1H), 10.76 (b, 2H), 4.15-4.10 (m, 4H), 2.37-2.32 (m, 2H).

### Step 3

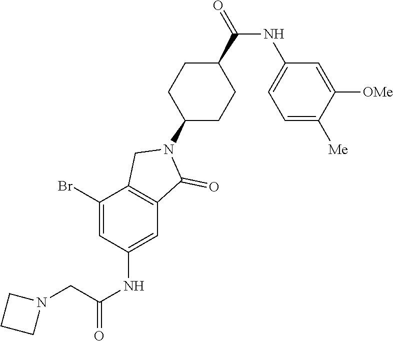 [SMILES: COc1cc(NC(=O)[C@H]2CC[C@@H](N3Cc4c(Br)cc(NC(=O)CN5CCC5)cc4C3=O)CC2)ccc1C]

### (1s,4s)-4-(6-(2-(Azetidin-1-yl) acetamido)-4-bromo-1-oxoisoindolin-2-yl)-N-(3-methoxy-4-methylphenyl)cyclohexanecarboxamide

A solution of 2-(azetidin-1-yl) acetic acid hydrochloride (24 mg, 210 μmol), (1s,4s)-4-(6-amino-4-bromo-1-oxoisoindolin-2-yl)-N-(3-methoxy-4-methylphenyl)cyclohexanecarboxamide (100 mg, 210 μmol), EDCl (49 mg, 250 μmol), DIPEA (82 mg, 640 μmol, 110 μL) and HOBt (34 mg, 250 μmol) in DMF (2 mL) was stirred at rt for 8 hours. The reaction mixture diluted in water (10 mL) and extracted with EtOAc (20 mL). The organic layer was dried over anhydrous Na₂SO₄, filtered and concentrated to get crude product, which was purified by prep-HPLC to give (1s,4s)-4-(6-(2-(azetidin-1-yl) acetamido)-4-bromo-1-oxoisoindolin-2-yl)-N-(3-methoxy-4-methylphenyl)cyclohexanecarboxamide (2.5 mg, 2.0% yield) as a white solid. LCMS (ESI) m/z: 568.7 [M+H]⁺. ¹H NMR (400 MHz, DMSO-d₆) δ 10.05 (b, 1H), 9.72 (b, 1H), 8.13 (s, 1H), 8.05 (s, 1H), 7.33 (s, 1H), 7.12 (d, J=7.6 Hz, 1H), 7.03 (d, J=7.7 Hz, 1H), 4.33 (s, 2H), 4.12-4.08 (m, 1H), 3.77 (s, 3H), 3.31-3.28 (m, 4H), 3.23 (s, 2H), 2.68-2.66 (m, 1H), 2.09-2.03 (m, 9H), 1.72-1.65 (m, 4H).

### Example 201

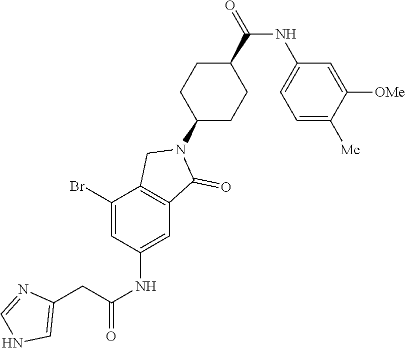 [SMILES: COc1cc(NC(=O)[C@H]2CC[C@@H](N3Cc4c(Br)cc(NC(=O)Cc5c[nH]cn5)cc4C3=O)CC2)ccc1C]

### (1s,4s)-4-(6-(2-(1H-Imidazol-4-yl) acetamido)-4-bromo-1-oxoisoindolin-2-yl)-N-(3-methoxy-4-methylphenyl)cyclohexanecarboxamide

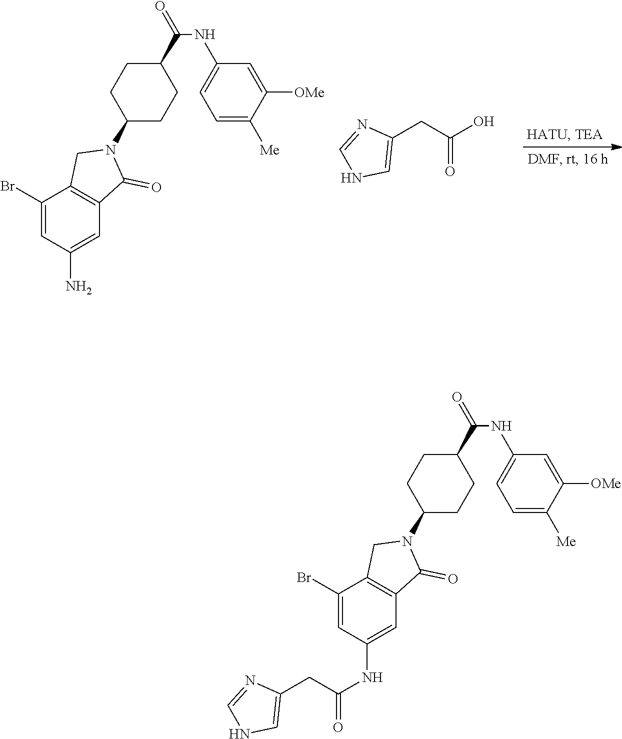 [SMILES: COc1cc(NC(=O)[C@H]2CC[C@@H](N3Cc4c(Br)cc(N)cc4C3=O)CC2)ccc1C ; O=C(O)Cc1c[nH]cn1 ; COc1cc(NC(=O)[C@H]2CC[C@@H](N3Cc4c(Br)cc(NC(=O)Cc5c[nH]cn5)cc4C3=O)CC2)ccc1C]

### Step 1

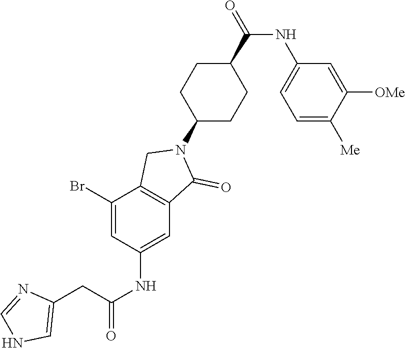 [SMILES: COc1cc(NC(=O)[C@H]2CC[C@@H](N3Cc4c(Br)cc(NC(=O)Cc5c[nH]cn5)cc4C3=O)CC2)ccc1C]

### (1s,4s)-4-(6-(2-(1H-Imidazol-4-yl) acetamido)-4-bromo-1-oxoisoindolin-2-yl)-N-(3-methoxy-4-methylphenyl)cyclohexanecarboxamide

Following the synthetic procedure for Example 196, but starting with 2-(1H-imidazol-4-yl) acetic acid gave the title compound as a white solid. LCMS (ESI) m/z: 581.7 [M+H]⁺. ¹H NMR (400 MHz, DMSO-d₆) 011.92 (s, 1H), 10.46 (s, 1H), 9.66 (s, 1H), 8.05 (d, J=1.6 Hz, 1H), 7.94 (d, J=1.6 Hz, 1H), 7.57 (s, 1H), 7.32 (d, J=1.6 Hz, 1H), 7.11 (dd, J=8.0, 1.7 Hz, 1H), 7.02 (d, J=8.2 Hz, 2H), 4.32 (s, 2H), 4.12-4.07 (m, 1H), 3.76 (s, 3H), 3.61-3.57 (m, 2H), 2.67-2.65 (m, 1H), 2.16-1.93 (m, 7H), 1.71-1.62 (m, 4H).

### Example 202

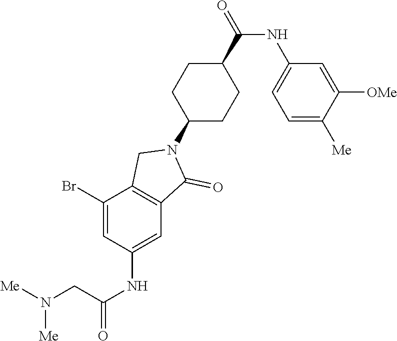 [SMILES: COc1cc(NC(=O)[C@H]2CC[C@@H](N3Cc4c(Br)cc(NC(=O)CN(C)C)cc4C3=O)CC2)ccc1C]

### (1s,4s)-4-(4-bromo-6-(2-(dimethylamino)acetamido)-1-oxoisoindolin-2-yl)-N-(3-methoxy-4-methylphenyl)cyclohexanecarboxamide

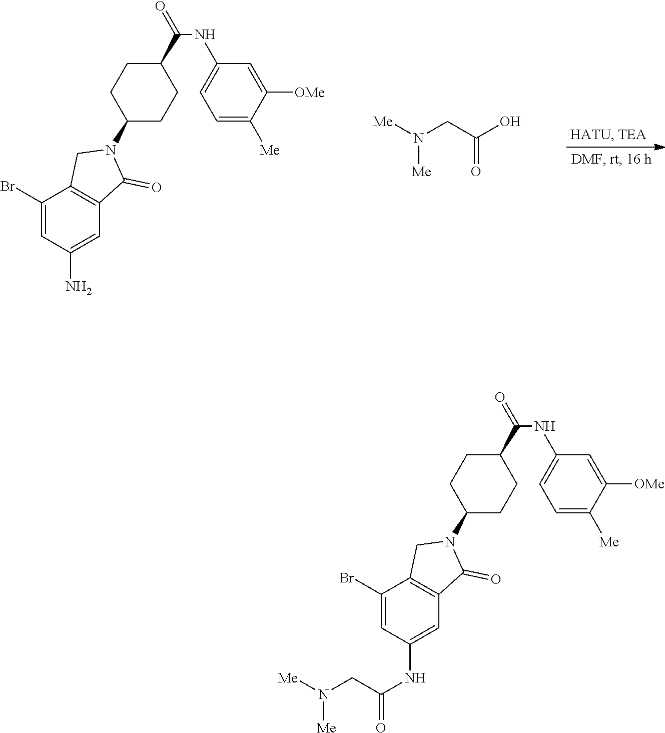 [SMILES: COc1cc(NC(=O)[C@H]2CC[C@@H](N3Cc4c(Br)cc(N)cc4C3=O)CC2)ccc1C ; CN(C)CC(=O)O ; COc1cc(NC(=O)[C@H]2CC[C@@H](N3Cc4c(Br)cc(NC(=O)CN(C)C)cc4C3=O)CC2)ccc1C]

### Step 1

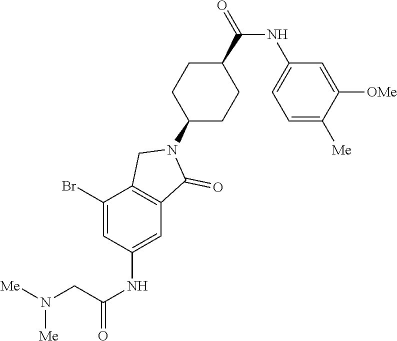 [SMILES: COc1cc(NC(=O)[C@H]2CC[C@@H](N3Cc4c(Br)cc(NC(=O)CN(C)C)cc4C3=O)CC2)ccc1C]

### (1s,4s)-4-(4-Bromo-6-(2-(dimethylamino)acetamido)-1-oxoisoindolin-2-yl)-N-(3-methoxy-4-methylphenyl)cyclohexanecarboxamide

Following the synthetic procedure for Example 196, but starting with 2-(dimethylamino)acetic acid gave the title compound as a white solid. LCMS (ESI) m/z: 558.7 [M+H]⁺. ¹H NMR (400 MHz, DMSO-d₆) δ 10.15 (s, 1H), 9.73 (s, 1H), 8.13 (d, J=1.6 Hz, 1H), 8.107 (d, J=1.6 Hz, 1H), 7.33 (d, J=1.5 Hz, 1H), 7.12 (dd, J=8.0, 1.7 Hz, 1H), 7.02 (d, J=8.2 Hz, 1H), 4.32 (s, 2H), 4.11-4.07 (m, 1H), 3.76 (s, 3H), 3.11 (s, 2H), 2.68-2.66 (m, 1H), 2.28 (s, 6H), 2.15-1.92 (m, 7H), 1.74-1.61 (m, 4H).

### Example 203

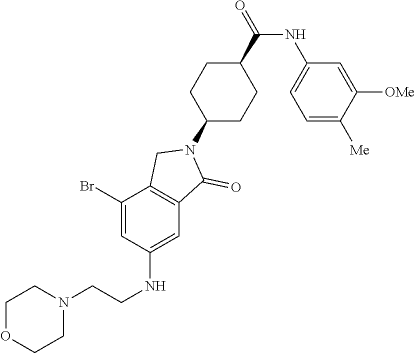 [SMILES: COc1cc(NC(=O)[C@H]2CC[C@@H](N3Cc4c(Br)cc(NCCN5CCOCC5)cc4C3=O)CC2)ccc1C]

### (1s,4s)-4-(4-Bromo-6-(2-morpholinoethylamino)-1-oxoisoindolin-2-yl)-N-(3-methoxy-4-methylphenyl)cyclohexanecarboxamide

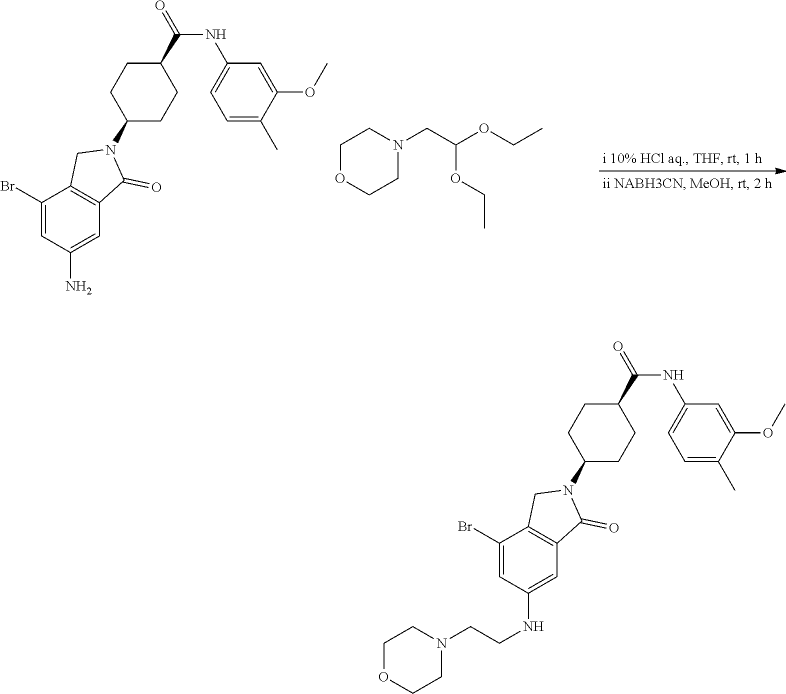 [SMILES: COc1cc(NC(=O)[C@H]2CC[C@@H](N3Cc4c(Br)cc(N)cc4C3=O)CC2)ccc1C ; CCOC(CN1CCOCC1)OCC ; COc1cc(NC(=O)[C@H]2CC[C@@H](N3Cc4c(Br)cc(NCCN5CCOCC5)cc4C3=O)CC2)ccc1C]

### Step 1

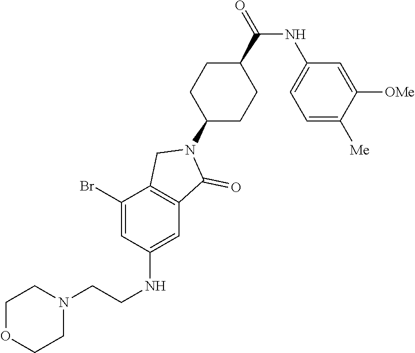 [SMILES: COc1cc(NC(=O)[C@H]2CC[C@@H](N3Cc4c(Br)cc(NCCN5CCOCC5)cc4C3=O)CC2)ccc1C]

### (1s,4s)-4-(4-Bromo-6-(2-morpholinoethylamino)-1-oxoisoindolin-2-yl)-N-(3-methoxy-4-methylphenyl)cyclohexanecarboxamide

Following the synthetic procedure for Example 198, but starting with 4-(2,2-diethoxyethyl)morpholine gave the title compound as a white solid. LCMS (ESI) m/z: 586.7 [M+H]⁺. ¹H NMR (400 MHz, DMSO-d₆) δ 9.72 (s, 1H), 7.32 (d, J=1.7 Hz, 1H), 7.12 (dd, J=8.0, 1.8 Hz, 1H), 7.05-7.01 (m, 2H), 6.83 (d, J=1.9 Hz, 1H), 6.03 (t, J=5.4 Hz, 1H), 4.19 (s, 2H), 4.09-4.05 (m, 1H), 3.76 (s, 3H), 3.62-3.56 (m, 4H), 3.17 (dd, J=12.3, 6.4 Hz, 2H), 2.67-2.65 (m, 1H), 2.50-2.47 (m, 2H), 2.47-2.36 (m, 4H), 2.12-2.07 (m, 5H), 2.01-1.96 (m, 2H), 1.75-1.70 (m, 2H), 1.65-1.57 (m, 2H).

### Example 204

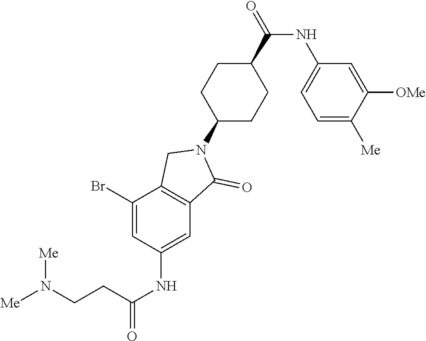 [SMILES: COc1cc(NC(=O)[C@H]2CC[C@@H](N3Cc4c(Br)cc(NC(=O)CCN(C)C)cc4C3=O)CC2)ccc1C]

### (1s,4s)-4-(4-Bromo-6-(3-(dimethylamino) propanamido)-1-oxoisoindolin-2-yl)-N-(3-methoxy-4-methylphenyl)cyclohexanecarboxamide

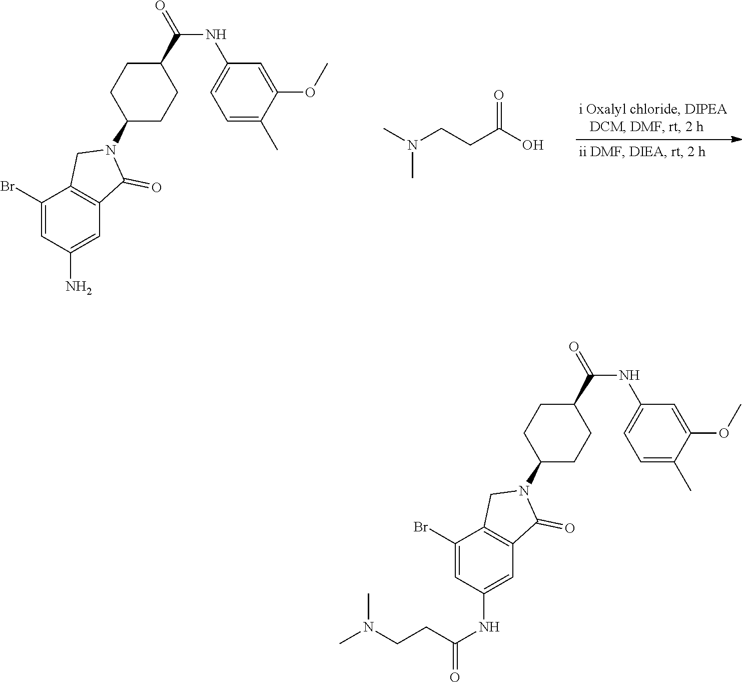

### Step 1

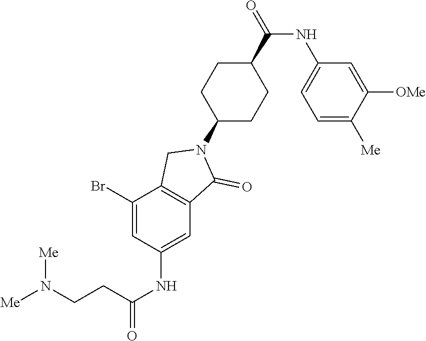 [SMILES: COc1cc(NC(=O)[C@H]2CC[C@@H](N3Cc4c(Br)cc(NC(=O)CCN(C)C)cc4C3=O)CC2)ccc1C]

### (1s,4s)-4-(4-Bromo-6-(3-(dimethylamino) propanamido)-1-oxoisoindolin-2-yl)-N-(3-methoxy-4-methylphenyl)cyclohexanecarboxamide

To a solution of 3-(dimethylamino) propanoic acid (300 mg, 2.6 mmol) and Oxalyl chloride (490 mg, 3.8 mmol, 340 μL) in DCM (6 mL) was added DMF (0.10 mL) at 0° C. under N₂. The mixture was stirred at 25° C. for 2 hours. The mixture was concentrated to give 3-(dimethylamino) propanoyl chloride (300 mg, crude) as a yellow solid. To a solution of 4-(6-amino-4-bromo-1-oxo-isoindolin-2-yl)-N-(3-methoxy-4-methyl-phenyl)cyclohexanecarboxamide (100 mg, 220 μmol) and 3-(dimethylamino) propanoyl chloride (300 mg, 2.2 mmol) in DMF (3.0 mL) was added DIPEA (290 mg, 2.2 mmol, 390 μL). The mixture was stirred at 25° C. for 2 hours. The mixture was poured into water and extracted with EtOAc. The organic extracts were washed with brine, dried by Na₂SO₄, filtered and concentrated to give a crude oil. The residue was purified by prep-HPLC to give (1s,4s)-4-(4-bromo-6-(3-(dimethylamino) propanamido)-1-oxoisoindolin-2-yl)-N-(3-methoxy-4-methylphenyl)cyclohexanecarboxamide (10 mg, 17 μmol, 7.6% yield) as a white solid. LCMS ESI m/z: 572.7 [M+H]⁺. ¹H NMR (400 MHz, DMSO-d₆) δ 10.36 (s, 1H), 9.71 (s, 1H), 8.03 (d, J=1.7 Hz, 1H), 7.92 (d, J=1.7 Hz, 1H), 7.32 (d, J=1.8 Hz, 1H), 7.11 (dd, J=8.0, 1.8 Hz, 1H), 7.02 (d, J=8.5 Hz, 1H), 4.32 (s, 2H), 4.11-4.07 (m, 1H), 3.76 (s, 3H), 2.67-2.65 (m, 1H), 2.58-2.54 (m, 2H), 2.49-2.45 (m, 2H), 2.17 (s, 6H), 2.12-2.08 (m, 5H), 2.06-1.95 (m, 2H), 1.76-1.59 (m, 4H).

### Example 205

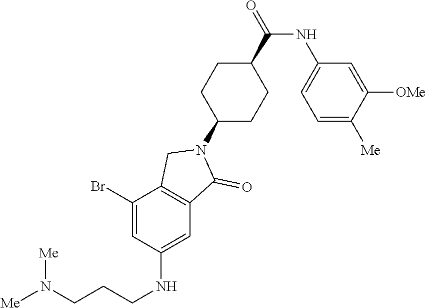 [SMILES: COc1cc(NC(=O)[C@H]2CC[C@@H](N3Cc4c(Br)cc(NCCCN(C)C)cc4C3=O)CC2)ccc1C]

### (1s,4s)-4-(4-Bromo-6-(3-(dimethylamino) propylamino)-1-oxoisoindolin-2-yl)-N-(3-methoxy-4-methylphenyl)cyclohexanecarboxamide

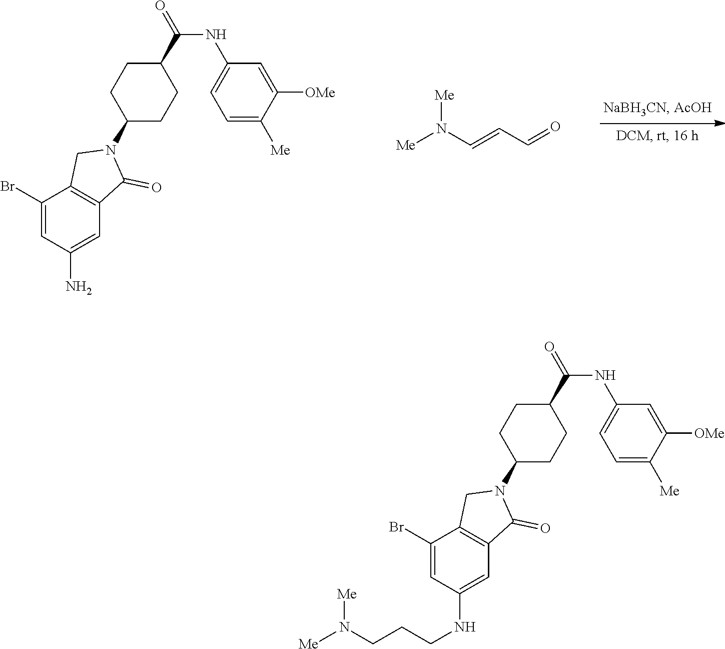 [SMILES: COc1cc(NC(=O)[C@H]2CC[C@@H](N3Cc4c(Br)cc(N)cc4C3=O)CC2)ccc1C ; CN(C)/C=C/C=O ; COc1cc(NC(=O)[C@H]2CC[C@@H](N3Cc4c(Br)cc(NCCCN(C)C)cc4C3=O)CC2)ccc1C]

### Step 1

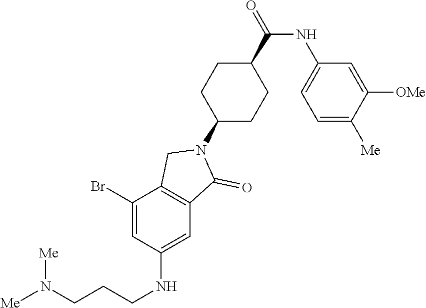 [SMILES: COc1cc(NC(=O)[C@H]2CC[C@@H](N3Cc4c(Br)cc(NCCCN(C)C)cc4C3=O)CC2)ccc1C]

### (1s,4s)-4-(4-Bromo-6-(3-(dimethylamino) propylamino)-1-oxoisoindolin-2-yl)-N-(3-methoxy-4-methylphenyl)cyclohexanecarboxamide

A solution of 4-(6-amino-4-bromo-1-oxo-isoindolin-2-yl)-N-(3-methoxy-4-methyl-phenyl)cyclohexanecarboxamide (120 mg, 250 μmol), (E)-3-(dimethylamino) prop-2-enal (730 mg, 7.4 mmol, 740 μL), acetic acid (290 mg, 4.9 mmol, 280 μL) and sodium cyanoborohydride (150 mg, 2.5 mmol) in DCM (20 mL) was stirred for 16 hours at 25° C. New spot was detected in TLC (DCM:MeOH=7:1, R_{f}=0.4). To the reaction mixture was added EtOAc (50 mL) and washed with H₂O (3×50 mL). The combined organic extracts were dried over Na₂SO₄ and evaporated in vacuo. The residue was purified by prep-TLC (DCM:MeOH=7:1, R_{f}=0.4) to get crude product, which was purified by Prep-HPLC to get (1s,4s)-4-(4-bromo-6-(3-(dimethylamino) propylamino)-1-oxoisoindolin-2-yl)-N-(3-methoxy-4-methylphenyl)cyclohexanecarboxamide (5.1 mg, 9.2 μmol, 3.7% yield) as white solid. LCMS ESI m/z: 556.8 [M+H]⁺. ¹H NMR (400 MHz, MeOD-d₄) δ 7.28 (d, J=1.6 Hz, 1H), 7.06-7.00 (m, 2H), 6.97-6.90 (m, 2H), 4.32 (s, 2H), 4.25-4.15 (m, 1H), 3.83 (s, 3H), 3.23 (t, J=6.8 Hz, 2H), 3.10-3.00 (m, 2H), 2.77-2.73 (m, 7H), 2.24-2.07 (m, 7H), 2.02-1.92 (m, 2H), 1.90-1.80 (m, 2H), 1.80-1.70 (m, 2H).

### Example 206

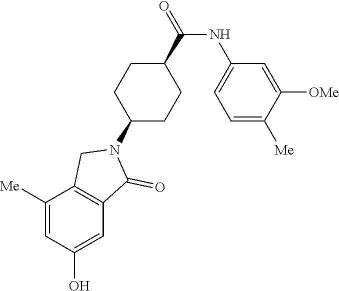 [SMILES: COc1cc(NC(=O)[C@H]2CC[C@@H](N3Cc4c(C)cc(O)cc4C3=O)CC2)ccc1C]

### (1s,4s)-4-(6-Hydroxy-4-methyl-1-oxoisoindolin-2-yl)-N-(3-methoxy-4-methylphenyl)cyclohexanecarboxamide

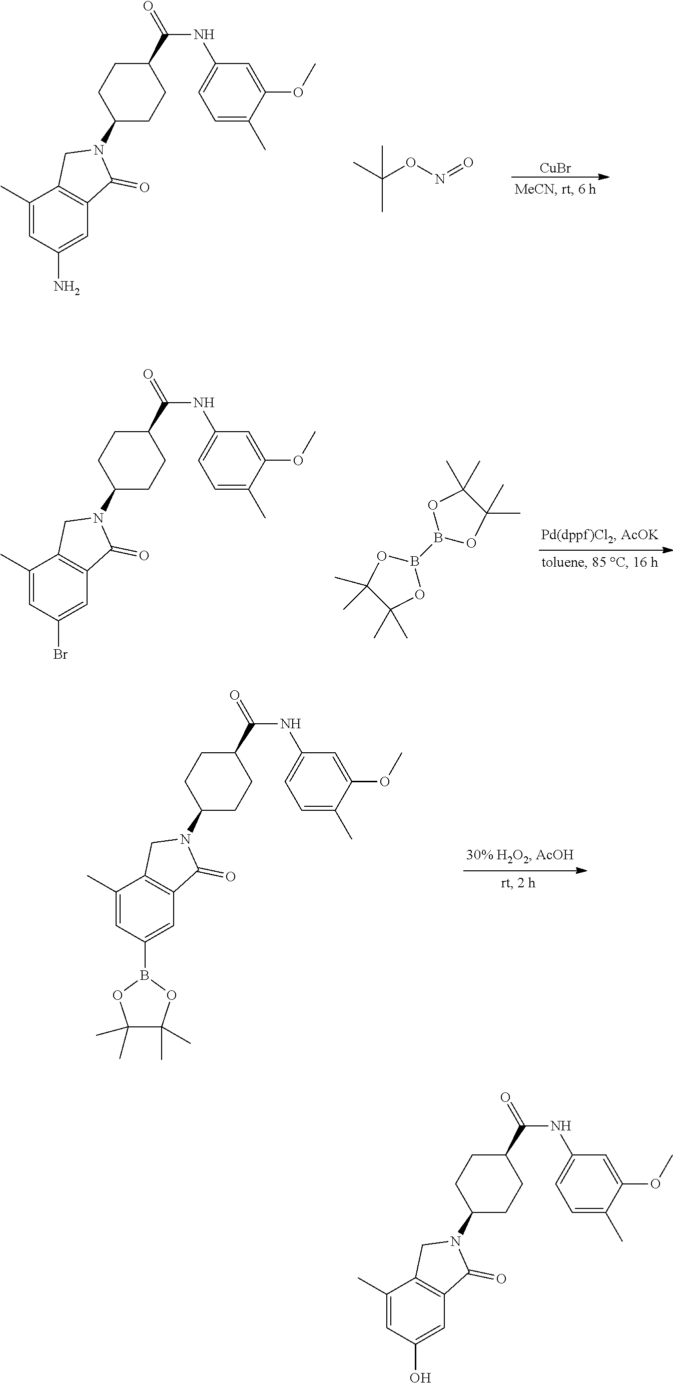

### Step 1

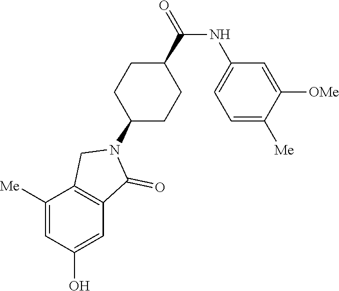 [SMILES: COc1cc(NC(=O)[C@H]2CC[C@@H](N3Cc4c(C)cc(O)cc4C3=O)CC2)ccc1C]

### (1s,4s)-4-(6-Bromo-4-methyl-1-oxoisoindolin-2-yl)-N-(3-methoxy-4-methylphenyl)cyclohexanecarboxamide

A mixture of 4-(6-amino-4-methyl-1-oxo-isoindolin-2-yl)-N-(3-methoxy-4-methyl-phenyl)cyclohexanecarboxamide (250 mg, 610 μmol) and copper (I) bromide (130 mg, 920 μmol,) in MeCN (5.0 mL) was added tert-butyl nitrite (95 mg, 920 μmol, 110 μL). The mixture was stirred at 25° C. for 6 hours. To the reaction solution was added HCl aq. (1M, 5.0 mL), then extracted with dichloromethane (50 mL) and washed with water (20 mL×3). The organic phases were dried over anhydrous sodium sulfate, filtered, and then evaporated to dryness. The residue was purified by silica gel chromatography to give (1s,4s)-4-(6-bromo-4-methyl-1-oxoisoindolin-2-yl)-N-(3-methoxy-4-methylphenyl)cyclohexanecarboxamide (100 mg, 170 μmol, 35% yield) as a yellow solid. LCMS ESI m/z: 473.1 [M+H]⁺.

### Step 2 and 3

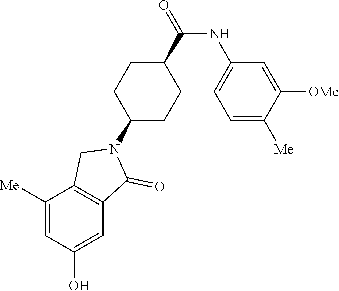 [SMILES: COc1cc(NC(=O)[C@H]2CC[C@@H](N3Cc4c(C)cc(O)cc4C3=O)CC2)ccc1C]

### (1s,4s)-4-(6-Hydroxy-4-methyl-1-oxoisoindolin-2-yl)-N-(3-methoxy-4-methylphenyl)cyclohexanecarboxamide

Following the synthetic procedure for Example 197, but starting with (1s,4s)-4-(6-bromo-4-methyl-1-oxoisoindolin-2-yl)-N-(3-methoxy-4-methylphenyl)cyclohexanecarboxamide gave the title compound as a white solid. LCMS (ESI) m/z: 408.9 [M+H]⁺. ¹H NMR (400 MHz, DMSO-d₆) δ 9.70 (s, 1H), 9.58 (b, 1H), 7.32 (d, J=1.7 Hz, 1H), 7.13 (dd, J=8.0, 1.8 Hz, 1H), 7.03 (d, J=8.1 Hz, 1H), 6.82 (d, J=2.0 Hz, 1H), 6.78 (d, J=1.5 Hz, 1H), 4.25 (s, 2H), 4.12-4.04 (m, 1H), 3.76 (s, 3H), 2.68-2.66 (m, 1H), 2.25 (s, 3H), 2.12-2.08 (m, 5H), 2.03-1.92 (m, 2H), 1.75-1.69 (m, 2H), 1.64-1.55 (m, 2H).

### Example 207

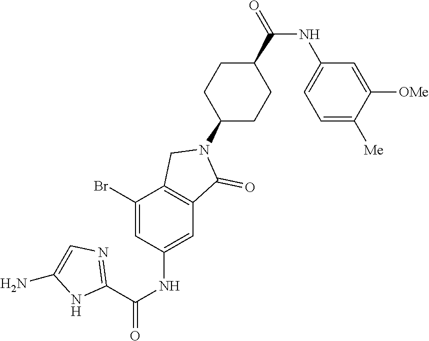 [SMILES: COc1cc(NC(=O)[C@H]2CC[C@@H](N3Cc4c(Br)cc(NC(=O)c5ncc(N)[nH]5)cc4C3=O)CC2)ccc1C]

### 2-Amino-N-(7-bromo-2-((1s,4s)-4-(3-methoxy-4-methylphenylcarbamoyl)cyclohexyl)-3-oxoisoindolin-5-yl)-1H-imidazole-4-carboxamide

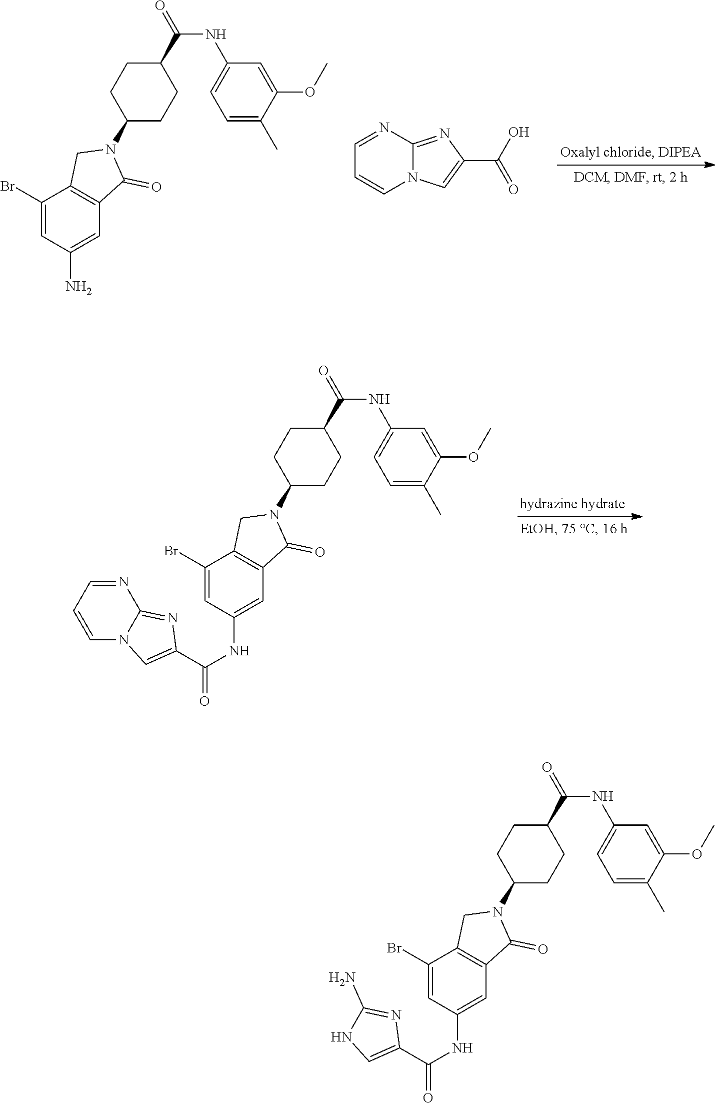 [SMILES: COc1cc(NC(=O)[C@H]2CC[C@@H](N3Cc4c(Br)cc(N)cc4C3=O)CC2)ccc1C ; O=C(O)c1cn2cccnc2n1 ; COc1cc(NC(=O)[C@H]2CC[C@@H](N3Cc4c(Br)cc(NC(=O)c5cn6cccnc6n5)cc4C3=O)CC2)ccc1C ; COc1cc(NC(=O)[C@H]2CC[C@@H](N3Cc4c(Br)cc(NC(=O)c5c[nH]c(N)n5)cc4C3=O)CC2)ccc1C]

### Step 1

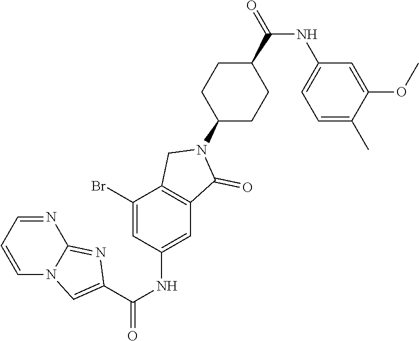 [SMILES: COc1cc(NC(=O)[C@H]2CC[C@@H](N3Cc4c(Br)cc(NC(=O)c5cn6cccnc6n5)cc4C3=O)CC2)ccc1C]

### N-(7-Bromo-2-((1s,4s)-4-(3-methoxy-4-methylphenylcarbamoyl)cyclohexyl)-3-oxoisoindolin-5-yl) imidazo[1,2-a]pyrimidine-2-carboxamide

To a solution of imidazo[1,2-a]pyrimidine-2-carboxylic acid (350 mg, 2.15 mmol) in DCM (10 mL) was added Oxalyl chloride (820 mg, 6.4 mmol, 560 μL) and DMF (0.10 mL) at 0° C. under N₂. The mixture was stirred at 25° C. for 2 hours. The mixture was concentrated to give imidazo[1,2-a]pyrimidine-2-carbonyl chloride (350 mg, crude) as a yellow oil. To a solution of 4-(6-amino-4-bromo-1-oxo-isoindolin-2-yl)-N-(3-methoxy-4-methyl-phenyl)cyclohexanecarboxamide (180 mg, 390 μmol), imidazo[1,2-a]pyrimidine-2-carbonyl chloride (350 mg, 1.9 mmol) in DMF (5 mL) was added DIPEA (400 mg, 3.01 mmol, 540 μL). The mixture was stirred at 25° C. for 2 hours. The mixture was poured into water and extracted with EtOAc. The organic extracts were washed with brine, dried by Na₂SO₄, filtered and concentrated to give a crude oil. The crude product was purified by Prep-TLC to give N-(7-bromo-2-((1s,4s)-4-(3-methoxy-4-methylphenylcarbamoyl)cyclohexyl)-3-oxoisoindolin-5-yl) imidazo[1,2-a]pyrimidine-2-carboxamide (100 mg, 97 μmol, 25% yield, 60% purity) as a yellow solid. LCMS ESI m/z: 616.6 [M+H]⁺.

### Step 2

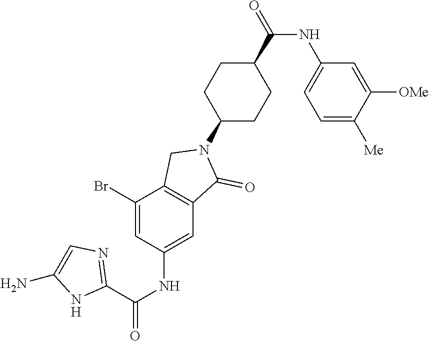 [SMILES: COc1cc(NC(=O)[C@H]2CC[C@@H](N3Cc4c(Br)cc(NC(=O)c5ncc(N)[nH]5)cc4C3=O)CC2)ccc1C]

### 2-Amino-N-(7-bromo-2-((1s,4s)-4-(3-methoxy-4-methylphenylcarbamoyl)cyclohexyl)-3-oxoisoindolin-5-yl)-1H-imidazole-4-carboxamide

To a solution of N-[7-bromo-2-[4-[(3-methoxy-4-methyl-phenyl)carbamoyl]cyclohexyl]-3-oxo-isoindolin-5-yl]imidazo[1,2-a]pyrimidine-2-carboxamide (100 mg, 160 μmol) in EtOH (4.0 mL) was added hydrazine monohydrate (12 mg, 240 μmol, 12 μL). The mixture was stirred at 75° C. for 2 hours. The mixture was concentrated to give a crude oil. The crude product was purified by prep-HPLC to give 2-amino-N-(7-bromo-2-((1s,4s)-4-(3-methoxy-4-methylphenylcarbamoyl)cyclohexyl)-3-oxoisoindolin-5-yl)-1H-imidazole-4-carboxamide (23 mg, 39 μmol, 24% yield) as a white solid. LCMS ESI m/z: 583.1 [M+H]⁺. ¹H NMR (400 MHz, DMSO-d₆) δ 11.99 (b, 1H), 10.31 (s, 1H), 9.73 (s, 1H), 8.19 (d, J=1.7 Hz, 1H), 8.13 (d, J=1.7 Hz, 1H), 7.65 (s, 1H), 7.33 (d, J=1.7 Hz, 1H), 7.12 (dd, J=8.0, 1.8 Hz, 1H), 7.02 (d, J=8.1 Hz, 1H), 6.61 (b, 1H), 4.34 (s, 2H), 4.12-4.08 (m, 1H), 3.76 (s, 3H), 2.68-2.66 (m, 1H), 2.12-2.08 (m, 5H), 2.04-2.00 (m, 2H), 1.77-1.60 (m, 4H).

### Example 208

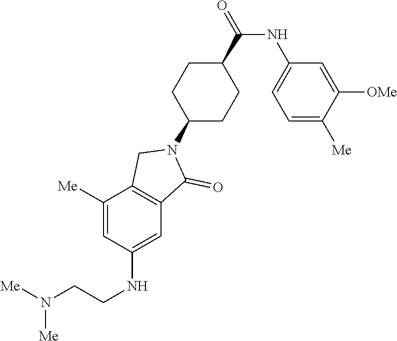 [SMILES: COc1cc(NC(=O)[C@H]2CC[C@@H](N3Cc4c(C)cc(NCCN(C)C)cc4C3=O)CC2)ccc1C]

### (1s,4s)-4-(6-(2-(Dimethylamino)ethylamino)-4-methyl-1-oxoisoindolin-2-yl)-N-(3-methoxy-4-methylphenyl)cyclohexanecarboxamide

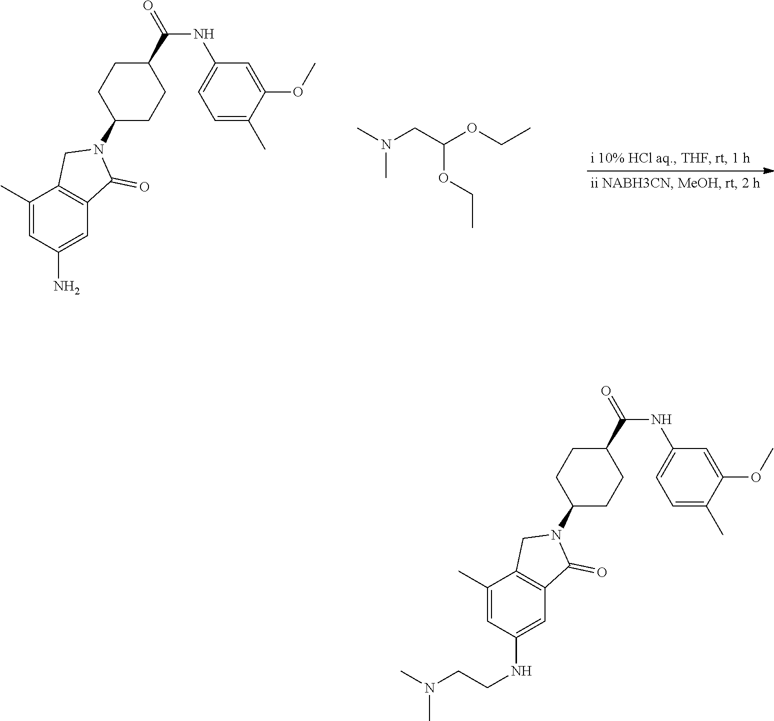

### Step 1

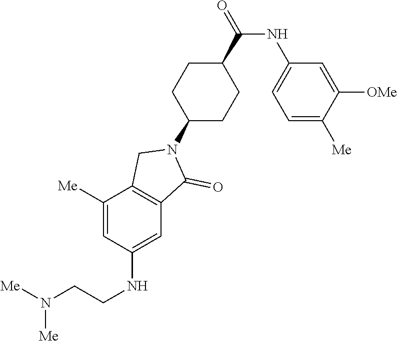 [SMILES: COc1cc(NC(=O)[C@H]2CC[C@@H](N3Cc4c(C)cc(NCCN(C)C)cc4C3=O)CC2)ccc1C]

### (1s,4s)-4-(6-(2-(Dimethylamino)ethylamino)-4-methyl-1-oxoisoindolin-2-yl)-N-(3-methoxy-4-methylphenyl)cyclohexanecarboxamide

Following the synthetic procedure for Example 198, but starting with (1s,4s)-4-(6-amino-4-methyl-1-oxoisoindolin-2-yl)-N-(3-methoxy-4-methylphenyl)cyclohexanecarboxamide gave the title compound as a white solid. LCMS (ESI) m/z: 478.9 [M+H]⁺. ¹H NMR (400 MHz, DMSO-d₆) δ 9.70 (s, 1H), 7.32 (d, J=1.7 Hz, 1H), 7.12 (dd, J=8.0, 1.8 Hz, 1H), 7.02 (d, J=8.1 Hz, 1H), 6.67-6.57 (m, 2H), 5.51 (t, J=5.5 Hz, 1H), 4.20 (s, 2H), 4.12-4.03 (m, 1H), 3.76 (s, 3H), 3.10 (dd, J=12.2, 6.3 Hz, 2H), 2.68-2.66 (m, 1H), 2.42 (t, J=6.6 Hz, 2H), 2.21 (s, 3H), 2.17 (s, 6H), 2.13-2.08 (m, 5H), 1.99-1.95 (m, 2H), 1.74-1.69 (m, 2H), 1.62-1.53 (m, 2H).

### Example 209

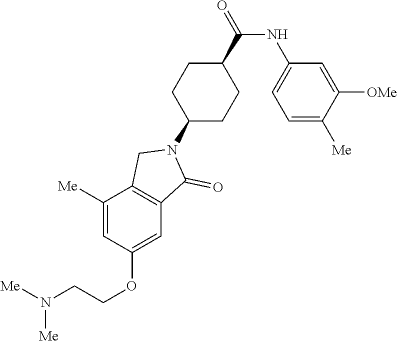 [SMILES: COc1cc(NC(=O)[C@H]2CC[C@@H](N3Cc4c(C)cc(OCCN(C)C)cc4C3=O)CC2)ccc1C]

### (1s,4s)-4-(6-(2-(Dimethylamino)ethoxy)-4-methyl-1-oxoisoindolin-2-yl)-N-(3-methoxy-4-methylphenyl)cyclohexanecarboxamide

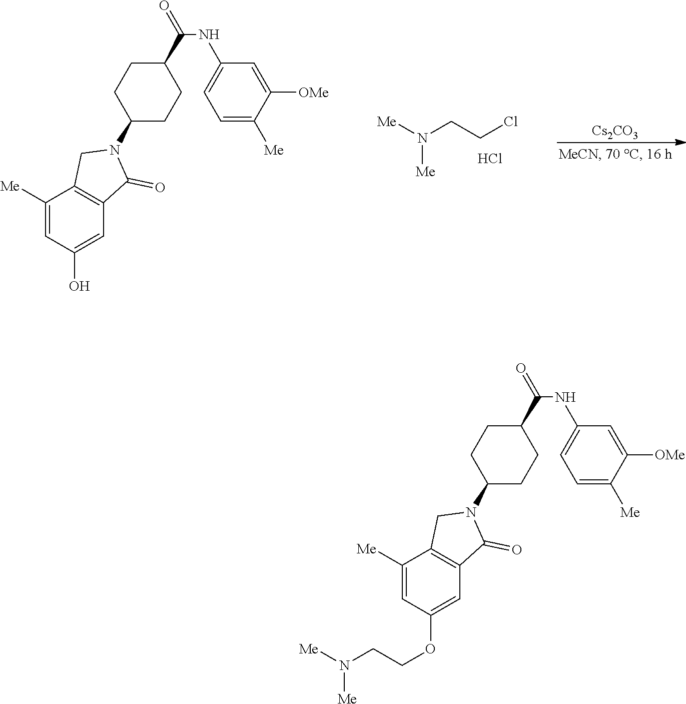

### Step 1

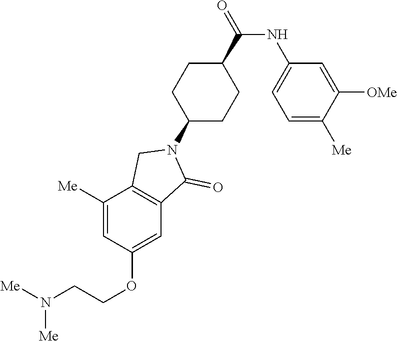 [SMILES: COc1cc(NC(=O)[C@H]2CC[C@@H](N3Cc4c(C)cc(OCCN(C)C)cc4C3=O)CC2)ccc1C]

### (1s,4s)-4-(6-(2-(Dimethylamino)ethoxy)-4-methyl-1-oxoisoindolin-2-yl)-N-(3-methoxy-4-methylphenyl)cyclohexanecarboxamide

A solution of 4-(6-hydroxy-4-methyl-1-oxo-isoindolin-2-yl)-N-(3-methoxy-4-methyl-phenyl)cyclohexanecarboxamide (50 mg, 120 μmol), 2-chloro-N,N-dimethyl-ethanamine (21 mg, 150 μmol, hydrochloride salt) and Cs₂CO₃ (80 mg, 240 μmol) in MeCN (2 mL) was stirred at 70° C. for 16 hours. The mixture was filtered and concentrated. The residue was purified by prep-HPLC to give (1s,4s)-4-(6-(2-(dimethylamino)ethoxy)-4-methyl-1-oxoisoindolin-2-yl)-N-(3-methoxy-4-methylphenyl)cyclohexanecarboxamide (11 mg, 22 μmol, 18% yield) as a white solid. LCMS ESI m/z 480.3 [M+H]⁺. ¹HNMR ¹H NMR (400 MHz, DMSO-d₆) δ 9.70 (s, 1H), 7.32 (d, J=1.7 Hz, 1H), 7.12 (dd, J=8.0, 1.8 Hz, 1H), 7.05-6.91 (m, 3H), 4.30 (s, 2H), 4.12-4.06 (m, 3H), 3.76 (s, 3H), 2.68-2.66 (m, 1H), 2.61 (t, J=5.8 Hz, 2H), 2.29 (s, 3H), 2.21 (s, 6H), 2.16-1.91 (m, 7H), 1.75-1.70 (m, 2H), 1.65-1.54 (m, 2H).

### Example 210

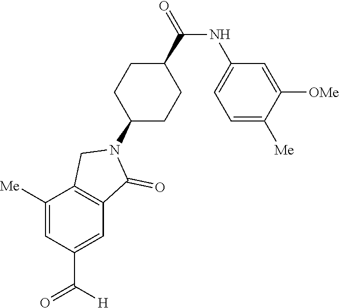 [SMILES: COc1cc(NC(=O)[C@H]2CC[C@@H](N3Cc4c(C)cc(C=O)cc4C3=O)CC2)ccc1C]

### (1s,4s)-4-(6-Formyl-4-methyl-1-oxoisoindolin-2-yl)-N-(3-methoxy-4-methylphenyl)cyclohexanecarboxamide

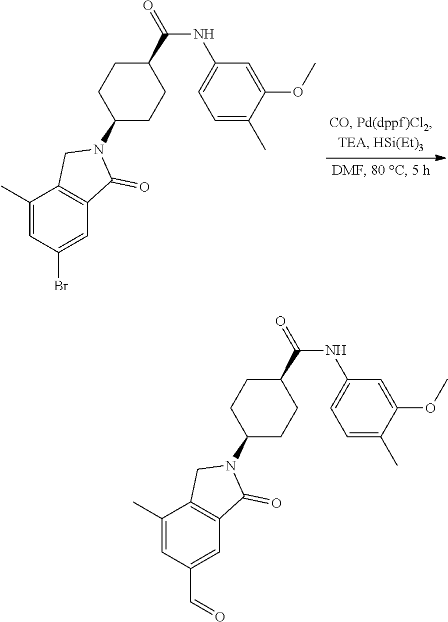

### Step 1

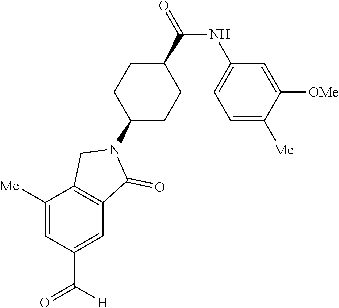 [SMILES: COc1cc(NC(=O)[C@H]2CC[C@@H](N3Cc4c(C)cc(C=O)cc4C3=O)CC2)ccc1C]

### (1s,4s)-4-(6-Formyl-4-methyl-1-oxoisoindolin-2-yl)-N-(3-methoxy-4-methylphenyl)cyclohexanecarboxamide

Following the synthetic procedure for Example 162, but starting with (1s,4s)-4-(6-bromo-4-methyl-1-oxoisoindolin-2-yl)-N-(3-methoxy-4-methylphenyl)cyclohexanecarboxamide gave the title compound as a white solid. LCMS (ESI) m/z: 420.9 [M+H]⁺. ¹H NMR (400 MHz, DMSO-d₆) δ 10.07 (s, 1H), 9.71 (s, 1H), 8.02 (s, 1H), 7.90 (s, 1H), 7.33 (d, J=1.6 Hz, 1H), 7.13 (dd, J=8.0, 1.8 Hz, 1H), 7.02 (d, J=8.3 Hz, 1H), 4.53 (s, 2H), 4.16-4.12 (m, 1H), 3.76 (s, 3H), 2.69-2.67 (m, 1H), 2.43 (s, 3H), 2.16-2.12 (m, 2H), 2.09-1.97 (m, 5H), 1.78-1.74 (m, 2H), 1.66-1.59 (m, 2H).

### Example 211

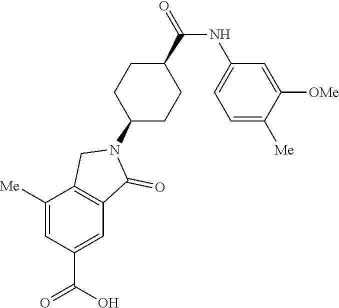 [SMILES: COc1cc(NC(=O)[C@H]2CC[C@@H](N3Cc4c(C)cc(C(=O)O)cc4C3=O)CC2)ccc1C]

### 2-((1s,4s)-4-(3-Methoxy-4-methylphenylcarbamoyl)cyclohexyl)-7-methyl-3-oxoisoindoline-5-carboxylic Acid

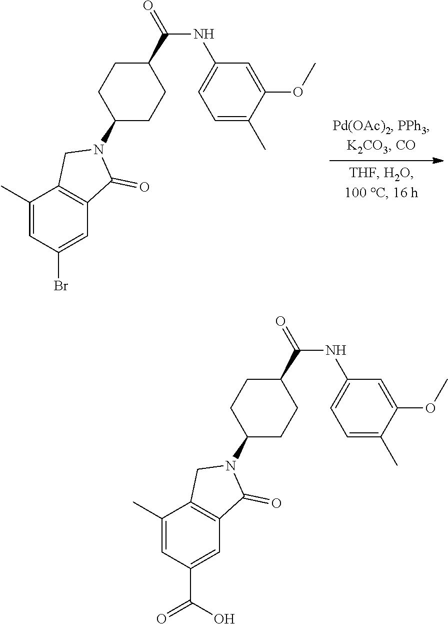

### Step 1

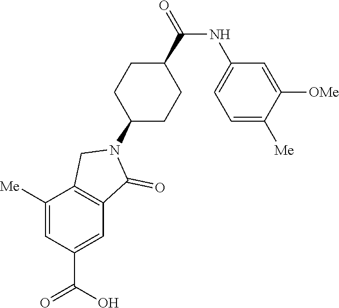 [SMILES: COc1cc(NC(=O)[C@H]2CC[C@@H](N3Cc4c(C)cc(C(=O)O)cc4C3=O)CC2)ccc1C]

### 2-((1s,4s)-4-(3-Methoxy-4-methylphenylcarbamoyl)cyclohexyl)-7-methyl-3-oxoisoindoline-5-carboxylic Acid

A mixture of 4-(6-bromo-4-methyl-1-oxo-isoindolin-2-yl)-N-(3-methoxy-4-methyl-phenyl)cyclohexanecarboxamide (120 mg, 250 μmol) in THF (6 mL) was added Pd(OAc)₂ (17 mg, 76 μmol), PPh₃ (41 mg, 150 μmol) and K₂CO₃ (110 mg, 760 μmol). The mixture was stirred at 100° C. for 16 hours under an atmosphere of carbon monoxide supplied by balloon. The mixture was poured into 1M HCl and extracted with EtOAc. The organic extracts were washed with brine, dried by Na₂SO₄, filtered and concentrated to give a crude oil. The crude product was purified by silica gel chromatography to give a yellow solid. The yellow solid was further purified by prep-HPLC to give 2-((1s,4s)-4-(3-methoxy-4-methylphenylcarbamoyl)cyclohexyl)-7-methyl-3-oxoisoindoline-5-carboxylic acid (15 mg, 34 μmol, 14% yield) as a white solid. LCMS ESI m/z: 436.9 [M+H]⁺. ¹H NMR (400 MHz, DMSO-d₆) δ 9.72 (s, 1H), 7.98 (s, 1H), 7.94 (s, 1H), 7.33 (d, J=1.7 Hz, 1H), 7.13 (dd, J=8.0, 1.8 Hz, 1H), 7.02 (d, J=8.5 Hz, 1H), 4.47 (s, 2H), 4.15-4.11 (m, 1H), 3.76 (s, 3H), 2.69-2.67 (m, 1H), 2.39 (s, 3H), 2.17-2.07 (m, 5H), 2.05-1.96 (m, 2H), 1.75-1.72 (m, 2H), 1.65-1.61 (m, 2H).

### Example 212

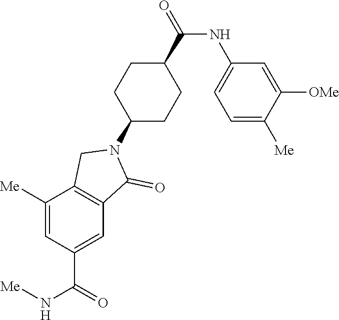 [SMILES: CNC(=O)c1cc(C)c2c(c1)C(=O)N([C@H]1CC[C@@H](C(=O)Nc3ccc(C)c(OC)c3)CC1)C2]

### 2-((1s,4s)-4-(3-Methoxy-4-methylphenylcarbamoyl)cyclohexyl)-N,7-dimethyl-3-oxoisoindoline-5-carboxamide

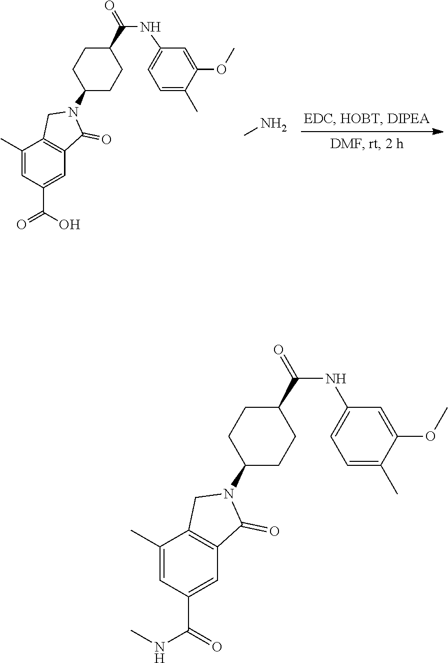

### Step 1

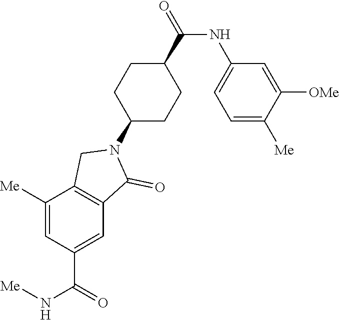 [SMILES: CNC(=O)c1cc(C)c2c(c1)C(=O)N([C@H]1CC[C@@H](C(=O)Nc3ccc(C)c(OC)c3)CC1)C2]

### 2-((1s,4s)-4-(3-Methoxy-4-methylphenylcarbamoyl)cyclohexyl)-N,7-dimethyl-3-oxoisoindoline-5-carboxamide

A mixture of 2-[4-[(3-methoxy-4-methyl-phenyl)carbamoyl]cyclohexyl]-7-methyl-3-oxo-isoindoline-5-carboxylic acid (70 mg, 160 μmol), DIPEA (62 mg, 480 μmol) and methanamine (7.47 mg, 240.55 μmol) in DMF (2.0 mL) was added EDC (30.74 mg, 160.37 μmol) and HOBT (65 mg, 480 μmol). The mixture was stirred at 25° C. for 2 hours. The mixture was poured into water and extracted with EtOAc. The organic extracts were washed with brine, dried by Na₂SO₄, filtered and concentrated to give a crude oil. The crude product was purified by prep-HPLC to give 2-((1s,4s)-4-(3-methoxy-4-methylphenylcarbamoyl)cyclohexyl)-N, 7-dimethyl-3-oxoisoindoline-5-carboxamide (11 mg, 24 μmol, 15% yield) as a white solid. LCMS ESI m/z: 449.9 [M+H]⁺. ¹H NMR (400 MHz, DMSO-d₆) δ 9.71 (s, 1H), 8.57 (d, J=4.4 Hz, 1H), 7.99 (s, 1H), 7.87 (s, 1H), 7.33 (d, J=1.3 Hz, 1H), 7.13 (dd, J=8.0, 1.6 Hz, 1H), 7.02 (d, J=8.1 Hz, 1H), 4.45 (s, 2H), 4.15-4.11 (m, 1H), 3.76 (s, 3H), 2.78 (d, J=4.4 Hz, 3H), 2.69-2.67 (m, 1H), 2.38 (s, 3H), 2.17-2.07 (m, 5H), 2.05-1.96 (m, 2H), 1.76-1.71 (m, 2H), 1.65-1.59 (m, 2H).

### Example 213

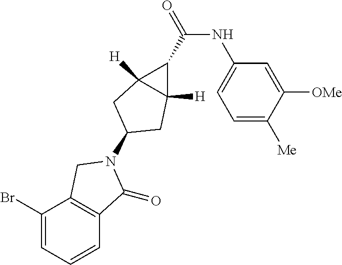 [SMILES: COc1cc(NC(=O)[C@@H]2[C@@H]3C[C@@H](N4Cc5c(Br)cccc5C4=O)C[C@@H]32)ccc1C]

### (1R,5S,6s)-3-(4-Bromo-1-oxoisoindolin-2-yl)-N-(3-methoxy-4-methylphenyl)bicyclo[3.1.0]hexane-6-carboxamide

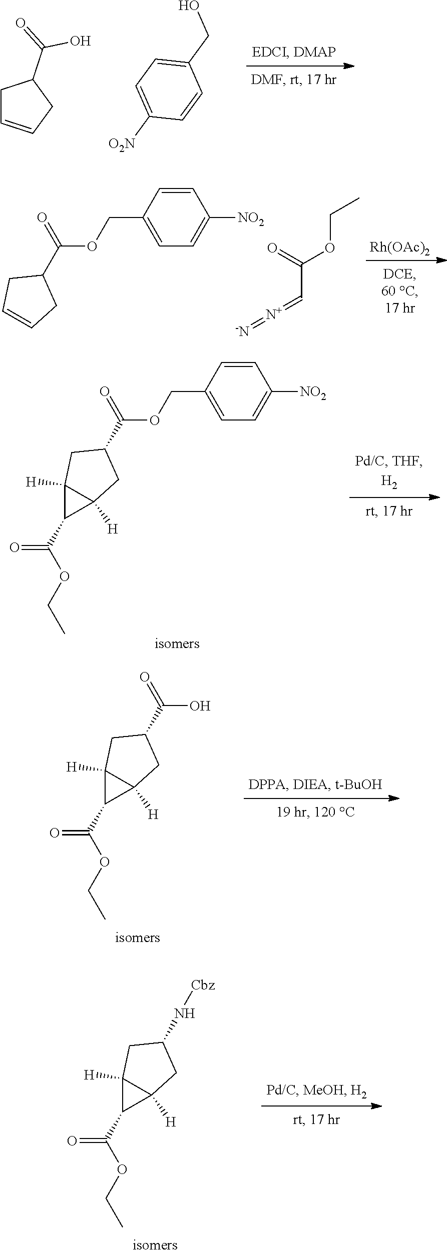

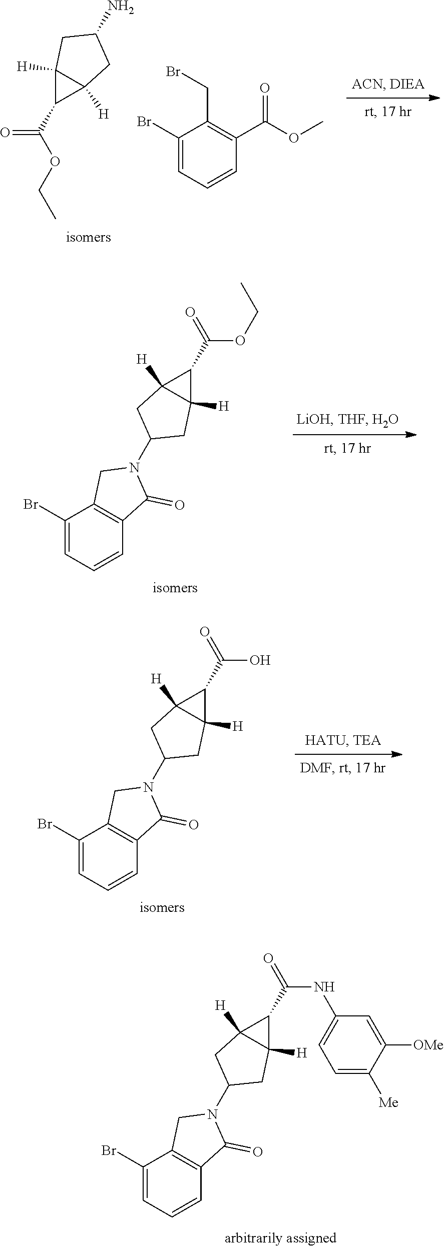

### Step 1

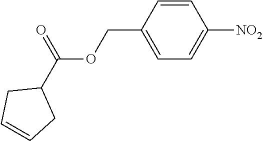 [SMILES: O=C(OCc1ccc([N+](=O)[O-])cc1)C1CC=CC1]

### 4-Nitrobenzyl cyclopent-3-enecarboxylate

To a solution of (4-nitrophenyl) methanol (7.58 g, 49.5 mmol) in DMF (8 mL) was added DMAP (8.1 g, 66 mmol) and EDCl (9.5 g, 50 mmol). The mixture was stirred at RT. Then cyclopent-3-ene-1-carboxylic acid (3.7 g, 33 mmol) was added to the solution with stirring at room temperature for 17 hours. The reaction was worked up with water and EtOAc. The organic layer was dried and concentrated. The residue was purified by column chromatography to give (4-nitrophenyl)methyl cyclopent-3-ene-1-carboxylate (7.0 g, 28 mmol, 86% yield) as a light-yellow oil. ¹HNMR (400 MHz, CDCl₃) δ 8.28-8.15 (m, 2H), 7.51 (d, J=8.8 Hz, 2H), 5.66 (s, 2H), 5.22 (s, 2H), 3.22-3.17 (m, 1H), 2.74-2.61 (m, 4H).

### Step 2

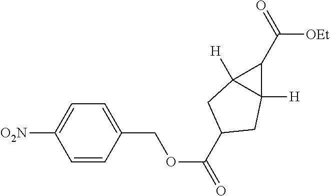 [SMILES: CCOC(=O)C1C2CC(C(=O)OCc3ccc([N+](=O)[O-])cc3)CC21]

### 6-Ethyl 3-(4-nitrobenzyl) bicyclo[3.1.0]hexane-3,6-dicarboxylate

To a solution of (4-nitrophenyl)methyl cyclopent-3-ene-1-carboxylate (7.0 g, 28 mmol) in 1,2-dichloroethane (200 mL) was added diacetoxyrhodium (130 mg, 570 μmol). The mixture was heated to 60° C. under N₂. Then ethyl 2-diazoacetate (16 g, 140 mmol) was added dropwise to the reaction mixture. The resulting mixture was stirred at 60° C. for another 17 hours. The mixture was filtered through a celite of pad and the filtrate was concentrated. The residue was purified by silica column (PE:EtOAc=30:1 to 10:1) to give a mixture of isomers 6-ethyl 3-(4-nitrobenzyl) bicyclo[3.1.0]hexane-3,6-dicarboxylate (800 mg, 2.5 mmol, 8.9% yield) as colorless oil. ¹HNMR (400 MHz, DMSO-d₆) δ 8.27-8.19 (m, 2H), 7.61 (d, J=8.8 Hz, 2H), 5.23 (s, 2H), 4.07 (q, J=7.1 Hz, 2H), 2.95-2.90 (m, 1H), 2.20-2.10 (m, 4H), 1.89-1.78 (m, 2H), 1.76-1.61 (m, 1H), 1.21 (t, J=7.1 Hz, 3H).

### Step 3

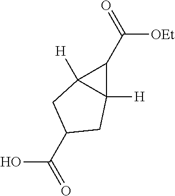 [SMILES: CCOC(=O)C1C2CC(C(=O)O)CC21]

### 6-(Ethoxycarbonyl)bicyclo[3.1.0]hexane-3-carboxylic Acid

To a solution of 6-ethyl 3-(4-nitrobenzyl) bicyclo[3.1.0]hexane-3,6-dicarboxylate (1.5 g, 4.5 mmol) in THF was added Pd/C (550 mg, 10% Pd wt.). The mixture was stirred at 25° C. under atmosphere of hydrogen gas supplied by a balloon for 17 hours. The mixture was filtered through a celite pad and concentrated. The residue was purified by silica gel chromatography to give a mixture of isomers 6-(ethoxycarbonyl)bicyclo[3.1.0]hexane-3-carboxylic acid (600 mg, 3.0 mmol, 67% yield) obtained as a yellow solid. ¹H NMR (400 MHz, DMSO-d₆) δ 12.06 (br, 1H), 4.06 (q, J=7.1 Hz, 2H), 2.64 (p, J=8.8 Hz, 1H), 2.16-1.95 (m, 4H), 1.81-1.78 (m, 2H), 1.71-1.58 (m, 1H), 1.19 (t, J=7.1 Hz, 3H).

### Step 4

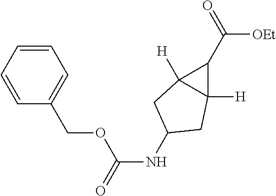 [SMILES: CCOC(=O)C1C2CC(NC(=O)OCc3ccccc3)CC21]

### Ethyl 3-(benzyloxycarbonylamino)bicyclo[3.1.0]hexane-6-carboxylate

A solution of isomers 6-(ethoxycarbonyl)bicyclo[3.1.0]hexane-3-carboxylic acid (600 mg, 3.0 mmol), [azido(phenoxy)phosphoryl]oxybenzene (1.0 mg, 3.6 mmol, 790 μL) and N-ethyl-N-isopropyl-propan-2-amine (470 mg, 3.6 mmol, 630 μL) in toluene (500 μL) was stirred at 120° C. for 2 hours, then phenylmethanol (390 mg, 3.6 mmol, 380 μL) was added. The mixture was stirred at 120° C. for 17 hours. The mixture was poured into water (50 mL) and extracted with EtOAc (30 mL*3). The organic phases were dried and concentrated in vacuo. The residue was purified by silica gel chromatography to give a mixture of isomers ethyl 3-(benzyloxycarbonylamino) bicyclo[3.1.0]hexane-6-carboxylate (600 mg, 1.98 mmol, 65.34% yield) as yellow oil. LCMS ESI m/z 304.2 [M+H]⁺.

### Step 5

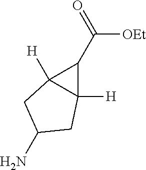 [SMILES: CCOC(=O)C1C2CC(N)CC21]

### Ethyl 3-aminobicyclo[3.1.0]hexane-6-carboxylate

A mixture of isomers ethyl 3-(benzyloxycarbonylamino) bicycle [3.1.0]hexane-6-carboxylate (600 mg, 2.0 mmol) and Pd/C (60 mg, 10% Pd wt.) and in MeOH (10 mL) was stirred at 25° C. for 17 hours under an atmosphere of hydrogen gas supplied by a balloon. The mixture was filtered through a celite pad and concentrated to give the crude product isomers ethyl 3-aminobicyclo[3.1.0]hexane-6-carboxylate as a yellow solid. The crude was used directly in next step without purification. LCMS ESI m/z 170.1 [M+H]⁺.

### Step 6

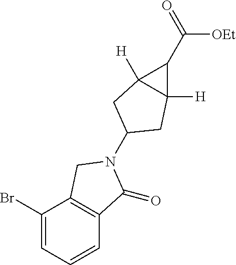 [SMILES: CCOC(=O)C1C2CC(N3Cc4c(Br)cccc4C3=O)CC21]

### Ethyl 3-(4-bromo-1-oxoisoindolin-2-yl)bicyclo[3.1.0]hexane-6-carboxylate

A mixture of isomers ethyl 3-aminobicyclo[3.1.0]hexane-6-carboxylate (600 mg, 3.6 mmol), methyl 3-bromo-2-(bromomethyl)benzoate (1.6 g, 5.3 mmol) and DIPEA (1.4 g, 11 mmol) in ACN (10 mL) was stirred at 25° C. for 17 hours. The mixture was poured into water (30 mL) and extracted with EtOAc (30 mL*3). The organic layers were dried and concentrated. The residue was purified by silica gel chromatography to give isomers ethyl 3-(4-bromo-1-oxoisoindolin-2-yl)bicyclo[3.1.0]hexane-6-carboxylate (400 mg, 1.1 mmol, 31% yield) as yellow oil. LCMS ESI m/z 364.0 [M+H]⁺.

### Step 7

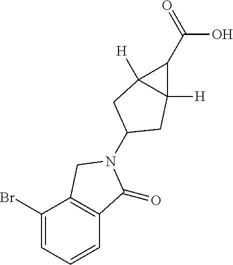 [SMILES: O=C(O)C1C2CC(N3Cc4c(Br)cccc4C3=O)CC21]

### 3-(4-Bromo-1-oxoisoindolin-2-yl)bicyclo[3.1.0]hexane-6-carboxylic Acid

To a solution of isomers ethyl 3-(4-bromo-1-oxo-isoindolin-2-yl)bicycle [3.1.0]hexane-6-carboxylate (700 mg, 1.60 mmol) in THF (10 mL) and H₂O (10 mL) was added LiOH (410 mg, 18 mmol). The mixture was stirred at 25° C. for 17 hours. To the mixture was added 1M HCl aq. until the solution reached pH~2. The solution was extracted with EtOAc (30 mL*3). The organic phases were dried and concentrated in vacuo. The residue was purified by silica gel chromatography to give isomers 3-(4-bromo-1-oxoisoindolin-2-yl)bicyclo[3.1.0]hexane-6-carboxylic acid (500 mg, 1.5 mmol, 94% yield) as a yellow solid. LCMS ESI m/z 335.7 [M+H].

### Step 8

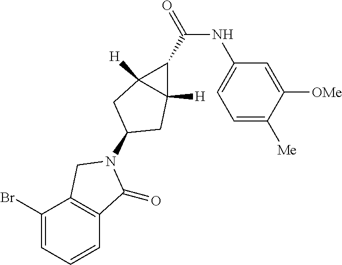 [SMILES: COc1cc(NC(=O)[C@@H]2[C@@H]3C[C@@H](N4Cc5c(Br)cccc5C4=O)C[C@@H]32)ccc1C]

### (1R,5S,6s)-3-(4-Bromo-1-oxoisoindolin-2-yl)-N-(3-methoxy-4-methylphenyl)bicyclo[3.1.0]hexane-6-carboxamide

To a solution of 3-(4-bromo-1-oxo-isoindolin-2-yl)bicyclo[3.1.0]hexane-6-carboxylic acid (60 mg, 0.17 mmol) in DMF (10 mL) was added HATU (81 mg, 0.21 mmol), TEA (54 mg, 0.53 mmol) and 3-methoxy-4-methyl-aniline (27 mg, 0.19 mmol). The mixture was stirred at 25° C. for 17 hours. The mixture was poured into water (20 mL) and extracted with EtOAc (30 mL). The organic layer was dried and concentrated. The residue was purified by prep-HPLC to give single isomer (1R,5S,6s)-3-(4-bromo-1-oxoisoindolin-2-yl)-N-(3-methoxy-4-methylphenyl)bicyclo[3.1.0]hexane-6-carboxamide (20 mg, 44 μmol, 25% yield) as a white solid. The stereochemistry was arbitrarily assigned (1R,5S,6s) and the isolated compound was racemic. LCMS ESI m/z 455.1 [M+H]⁺. ¹H NMR (400 MHz, DMSO-d₆) δ 9.94 (s, 1H), 7.79 (dd, J=7.9, 0.7 Hz, 1H), 7.64 (d, J=6.9 Hz, 1H), 7.44 (t, J=7.7 Hz, 1H), 7.31 (d, J=1.5 Hz, 1H), 7.09 (d, J=1.7 Hz, 1H), 7.02 (d, J=8.1 Hz, 1H), 4.79-4.75 (m, 1H), 4.44 (s, 2H), 3.77 (s, 3H), 2.29-2.06 (m, 7H), 1.92-1.82 (m, 2H), 1.79-1.75 (m, 1H).

### Example 214

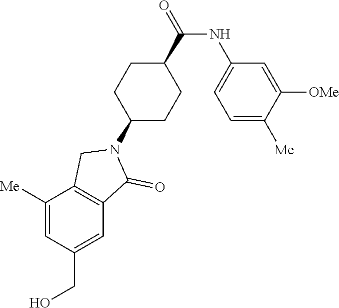 [SMILES: COc1cc(NC(=O)[C@H]2CC[C@@H](N3Cc4c(C)cc(CO)cc4C3=O)CC2)ccc1C]

### (1s,4s)-4-(6-(Hydroxymethyl)-4-methyl-1-oxoisoindolin-2-yl)-N-(3-methoxy-4-methylphenyl)cyclohexanecarboxamide

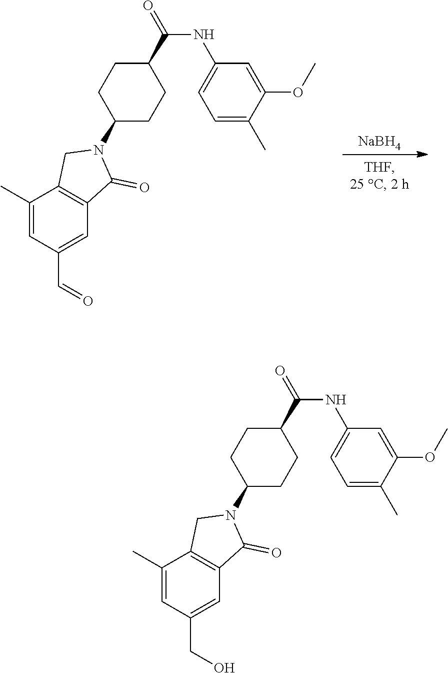

### Step 1

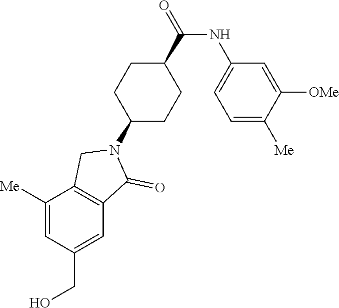 [SMILES: COc1cc(NC(=O)[C@H]2CC[C@@H](N3Cc4c(C)cc(CO)cc4C3=O)CC2)ccc1C]

### (1s,4s)-4-(6-(Hydroxymethyl)-4-methyl-1-oxoisoindolin-2-yl)-N-(3-methoxy-4-methylphenyl)cyclohexanecarboxamide

A solution of 4-(6-formyl-4-methyl-1-oxo-isoindolin-2-yl)-N-(3-methoxy-4-methyl-phenyl)cyclohexanecarboxamide (75 mg, 180 μmol) in THF (0.5 mL) was added NaBH₄ (34 mg, 890 μmol). The mixture was stirred at 25° C. for 2 hours. The mixture was poured into water and extracted with EtOAc. The organic extracts were washed with brine, dried by Na₂SO₄, filtered and concentrated to give a crude oil. The crude product was purified by prep-HPLC to give (1s,4s)-4-(6-(hydroxymethyl)-4-methyl-1-oxoisoindolin-2-yl)-N-(3-methoxy-4-methylphenyl)cyclohexanecarboxamide (16 mg, 38 μmol, 21% yield, 100%) as a white solid. LCMS ESI m/z: 423.3 [M+H]⁺. ¹H NMR (400 MHz, DMSO-d₆) δ 9.70 (s, 1H), 7.43 (s, 1H), 7.35-7.28 (m, 2H), 7.13 (dd, J=8.0, 1.6 Hz, 1H), 7.02 (d, J=8.1 Hz, 1H), 5.25 (br, 1H), 4.54 (s, 2H), 4.36 (s, 2H), 4.13-4.09 (m, 1H), 3.76 (s, 3H), 2.69-2.67 (m, 1H), 2.32 (s, 3H), 2.19-2.07 (m, 5H), 2.04-1.96 (m, 2H), 1.77-1.67 (m, 2H), 1.63-1.58 (m, 2H).

### Example 215

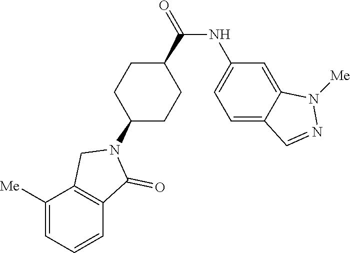 [SMILES: Cc1cccc2c1CN([C@H]1CC[C@@H](C(=O)Nc3ccc4cnn(C)c4c3)CC1)C2=O]

### (1s,4s)-4-(4-Methyl-1-oxoisoindolin-2-yl)-N-(1-methyl-1H-indazol-6-yl)cyclohexanecarboxamide

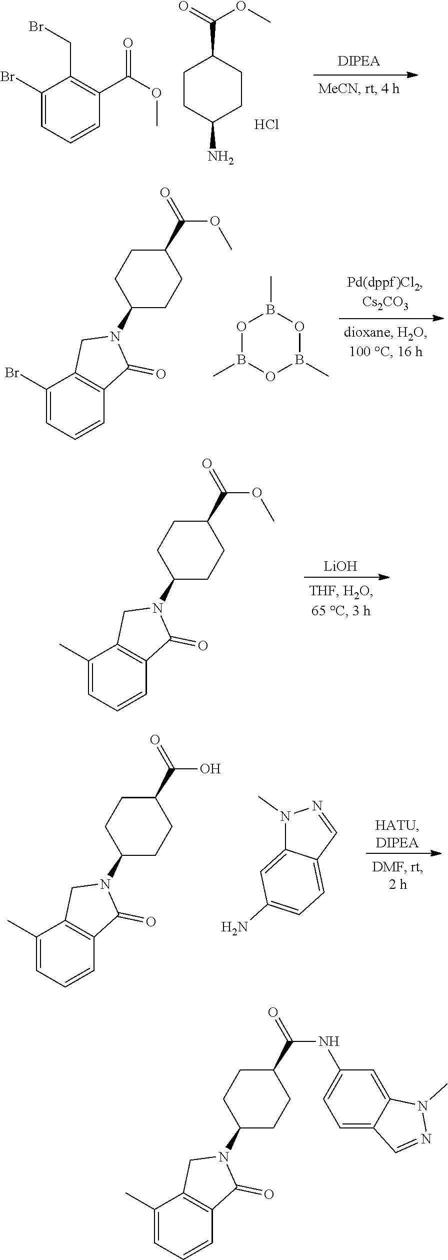

### Step 1

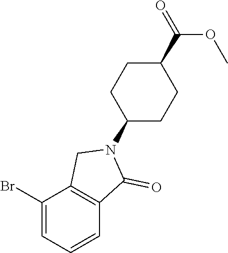 [SMILES: COC(=O)[C@H]1CC[C@@H](N2Cc3c(Br)cccc3C2=O)CC1]

### (1s,4s)-Methyl 4-(4-bromo-1-oxoisoindolin-2-yl)cyclohexanecarboxylate

To a solution of methyl 3-bromo-2-(bromomethyl)benzoate (5.0 g, 16 mmol) and methyl 4-aminocyclohexanecarboxylate (2.55 g, 16.2 mmol) in MeCN (100 mL) was added DIPEA (6.29 g, 48.7 mmol, 8.48 mL). The mixture was stirred at 25° C. for 4 hours. The mixture was worked up with water and EtOAc. The organic layer was dried and concentrated to give a crude oil. The crude product was purified by silica gel chromatography to give (1s,4s)-methyl 4-(4-bromo-1-oxoisoindolin-2-yl)cyclohexanecarboxylate (4.0 g, 10 mmol, 63% yield) as a yellow solid. LCMS ESI m/z: 354.1 [M+H]⁺.

### Step 2

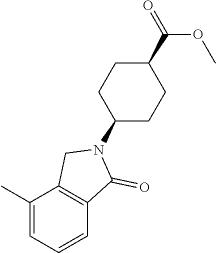

### (1s,4s)-Methyl 4-(4-methyl-1-oxoisoindolin-2-yl)cyclohexanecarboxylate

A mixture of methyl 4-(4-bromo-1-oxo-isoindolin-2-yl)cyclohexanecarboxylate (2.4 g, 6.8 mmol), 2,4,6-trimethyl-1,3,5,2,4,6-trioxatriborinane (1.7 g, 14 mmol), Pd(dppf)Cl₂ (500 mg, 680 μmol) and Cs₂CO₃ (6.7 g, 20 mmol) in dioxane (30 mL)/H₂O (10 mL) was stirred at 100° C. for 16 hours under nitrogen. The mixture was poured into water and extracted with EtOAc. The organic extracts were washed with brine, dried by Na₂SO₄, filtered and concentrated to give a crude oil. The crude product was purified by silica gel chromatography to give (1s,4s)-methyl 4-(4-methyl-1-oxoisoindolin-2-yl)cyclohexanecarboxylate (1.6 g, 5.0 mmol, 74% yield, 90% purity) as a yellow solid. LCMS ESI m/z: 288.2 [M+H]⁺.

### Step 3

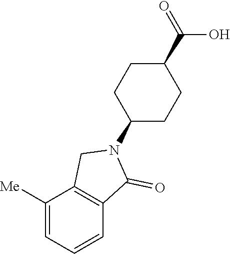 [SMILES: Cc1cccc2c1CN([C@H]1CC[C@@H](C(=O)O)CC1)C2=O]

### (1s,4s)-4-(4-Methyl-1-oxoisoindolin-2-yl)cyclohexanecarboxylic Acid

A mixture of methyl 4-(4-methyl-1-oxo-isoindolin-2-yl)cyclohexanecarboxylate (1.6 g, 5.6 mmol), LiOH (670 mg, 28 mmol) in THF (20 mL)/H₂O (5 mL) was stirred at 65° C. for 3 hours. The mixture was acidified with 1M HCl and extracted with EtOAc. The organic extracts were washed with brine, dried by Na₂SO₄, filtered and concentrated to give (1s,4s)-4-(4-methyl-1-oxoisoindolin-2-yl)cyclohexanecarboxylic acid (1.2 g, 4.0 mmol, 71% yield) as a yellow solid. LCMS ESI m/z: 274.2 [M+H]⁺.

### Step 4

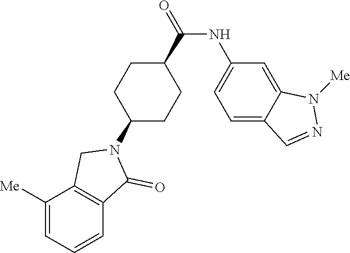 [SMILES: Cc1cccc2c1CN([C@H]1CC[C@@H](C(=O)Nc3ccc4cnn(C)c4c3)CC1)C2=O]

### (1s,4s)-4-(4-Methyl-1-oxoisoindolin-2-yl)-N-(1-methyl-1H-indazol-6-yl)cyclohexanecarboxamide

A mixture of (1s,4s)-4-(4-methyl-1-oxoisoindolin-2-yl)cyclohexanecarboxylic acid (50 mg, 180 μmol), HATU (83 mg, 220 μmol) in DMF (2.0 mL) was stirred at 25° C. for 30 minutes. Then 1-methylindazol-6-amine (40 mg, 270 μmol) was added. The solution was stirred at 25° C. for 2 hours. The mixture was poured into water and extracted with EtOAc. The organic extracts were washed with brine, dried by Na₂SO₄, filtered and concentrated to give a crude oil. The crude product was purified by prep-HPLC to give (1s,4s)-4-(4-methyl-1-oxoisoindolin-2-yl)-N-(1-methyl-1H-indazol-6-yl)cyclohexanecarboxamide (20 mg, 50 μmol, 27% yield) as a white solid. LCMS ESI m/z: 402.9 [M+H]⁺. 1H NMR (400 MHz, DMSO-d₆) δ 10.00 (s, 1H), 8.19 (s, 1H), 7.93 (d, J=0.9 Hz, 1H), 7.65 (d, J=8.6 Hz, 1H), 7.51-7.46 (m, 1H), 7.40-7.36 (m, 2H), 7.16 (dd, J=8.7, 1.6 Hz, 1H), 4.40 (s, 2H), 4.18-4.10 (m, 1H), 3.98 (s, 3H), 2.77-2.75 (m, 1H), 2.34 (s, 3H), 2.19-2.15 (m, 2H), 2.09-2.01 (m, 2H), 1.79-1.74 (m, 2H), 1.68-1.60 (m, 2H).

### Example 216

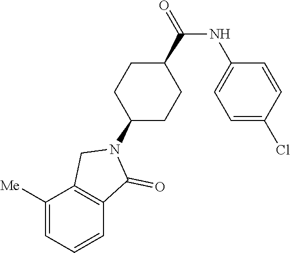 [SMILES: Cc1cccc2c1CN([C@H]1CC[C@@H](C(=O)Nc3ccc(Cl)cc3)CC1)C2=O]

### (1s,4s)-4-(4-Methyl-1-oxoisoindolin-2-yl)-N-(1-methyl-1H-indazol-6-yl)cyclohexanecarboxamide

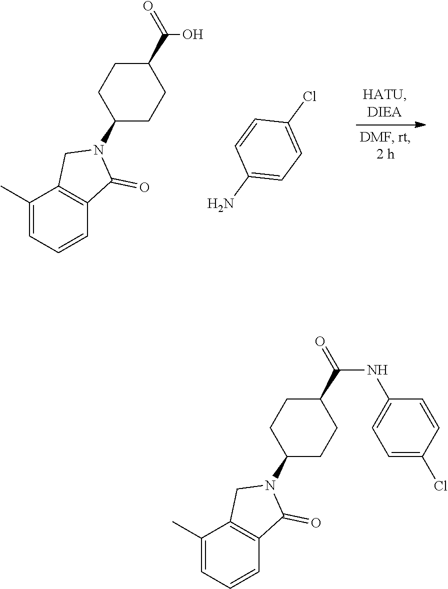 [SMILES: Cc1cccc2c1CN([C@H]1CC[C@@H](C(=O)O)CC1)C2=O ; Nc1ccc(Cl)cc1 ; Cc1cccc2c1CN([C@H]1CC[C@@H](C(=O)Nc3ccc(Cl)cc3)CC1)C2=O]

### Step 1

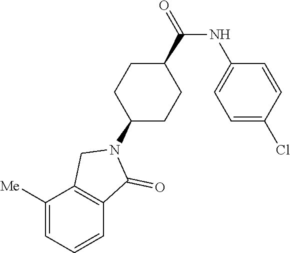 [SMILES: Cc1cccc2c1CN([C@H]1CC[C@@H](C(=O)Nc3ccc(Cl)cc3)CC1)C2=O]

### (1s,4s)-N-(4-Chlorophenyl)-4-(4-methyl-1-oxoisoindolin-2-yl)cyclohexanecarboxamide

Following the synthetic procedure for Example 215, but starting with 4-chloroaniline gave the title compound as a white solid. LC/MS 383.2 [M+H]⁺. ¹H NMR (400 MHz, CDCl₃) δ 7.67 (d, J=7.3 Hz, 1H), 7.51 (d, J=8.8 Hz, 2H), 7.39-7.35 (m, 2H), 7.33-7.28 (m, 3H), 4.37-4.33 (m, 1H), 4.29 (s, 2H), 2.69-2.67 (m, 1H), 2.34 (s, 3H), 2.24-2.20 (m, 2H), 2.13-2.01 (m, 2H), 1.93-1.75 (m, 4H).

### Example 217

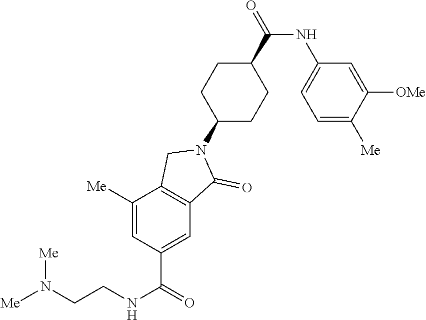 [SMILES: COc1cc(NC(=O)[C@H]2CC[C@@H](N3Cc4c(C)cc(C(=O)NCCN(C)C)cc4C3=O)CC2)ccc1C]

### N-(2-(Dimethylamino)ethyl)-2-((1s,4s)-4-(3-methoxy-4-methylphenylcarbamoyl)cyclohexyl)-7-methyl-3-oxoisoindoline-5-carboxamide

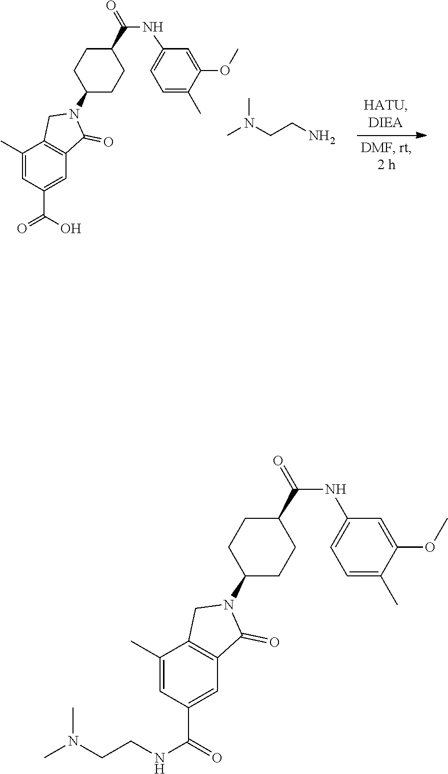

### Step 1

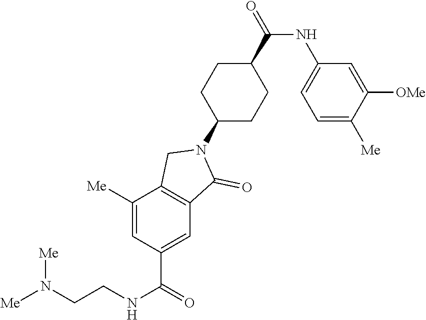 [SMILES: COc1cc(NC(=O)[C@H]2CC[C@@H](N3Cc4c(C)cc(C(=O)NCCN(C)C)cc4C3=O)CC2)ccc1C]

### N-(2-(Dimethylamino)ethyl)-2-((1s,4s)-4-(3-methoxy-4-methylphenylcarbamoyl)cyclohexyl)-7-methyl-3-oxoisoindoline-5-carboxamide

Following the synthetic procedure for Example 215, but starting with 2-((1s,4s)-4-(3-methoxy-4-methylphenylcarbamoyl)cyclohexyl)-7-methyl-3-oxoisoindoline-5-carboxylic acid and N1,N1-dimethylethane-1,2-diamine gave the title compound as a white solid. LC/MS 506.8 [M+H]⁺. ¹H NMR (400 MHz, DMSO-d₆) δ 9.72 (s, 1H), 8.54 (t, J=5.3 Hz, 1H), 8.01 (s, 1H), 7.87 (s, 1H), 7.33 (s, 1H), 7.13 (d, J=7.8 Hz, 1H), 7.03 (d, J=8.1 Hz, 1H), 4.46 (s, 2H), 4.16-4.12 (m, 1H), 3.76 (s, 3H), 2.70-2.68 (m, 1H), 2.52-2.48 (m, 2H), 2.44-2.36 (m, 5H), 2.18 (s, 6H), 2.16-2.07 (m, 5H), 2.06-1.96 (m, 2H), 1.78-1.69 (m, 2H), 1.66-1.59 (m, 2H).

### Example 218

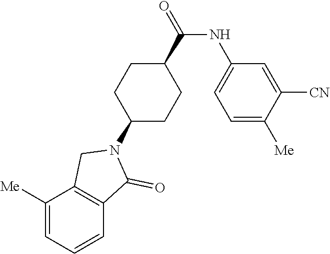 [SMILES: Cc1ccc(NC(=O)[C@H]2CC[C@@H](N3Cc4c(C)cccc4C3=O)CC2)cc1C#N]

### (1s,4s)-N-(3-Cyano-4-methylphenyl)-4-(4-methyl-1-oxoisoindolin-2-yl)cyclohexanecarboxamide

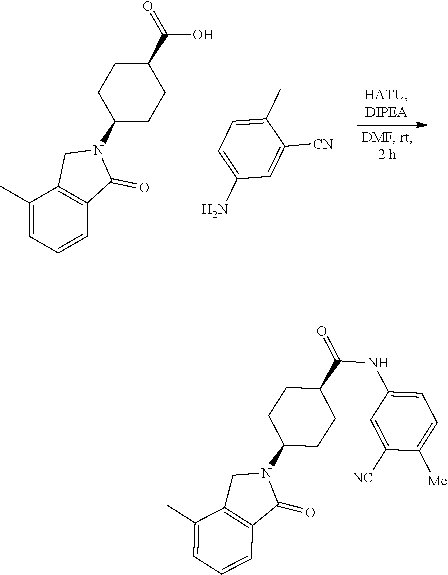

### Step 1

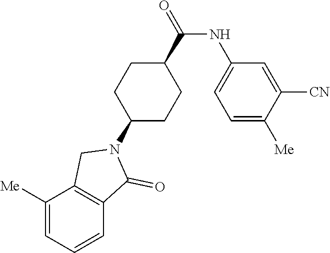 [SMILES: Cc1ccc(NC(=O)[C@H]2CC[C@@H](N3Cc4c(C)cccc4C3=O)CC2)cc1C#N]

### (1s,4s)-N-(3-Cyano-4-methylphenyl)-4-(4-methyl-1-oxoisoindolin-2-yl)cyclohexanecarboxamide

Following the general synthetic procedure for Example 215, but starting with 5-amino-2-methylbenzonitrile gave the title compound as a white solid. LC/MS ESI 377.0 [M+H]⁺. ¹H NMR (400 MHz, DMSO-d₆) δ9.64 (s, 1H), 7.53 (d, J=9.0 Hz, 2H), 7.50-7.45 (m, 1H), 7.38-7.35 (m, 2H), 6.89-6.83 (m, 2H), 4.38 (s, 2H), 4.13-4.09 (m, 1H), 3.72 (s, 3H), 2.67-2.65 (m, 1H), 2.34 (s, 3H), 2.16-1.95 (m, 4H), 1.77-1.57 (m, 4H).

### Example 219

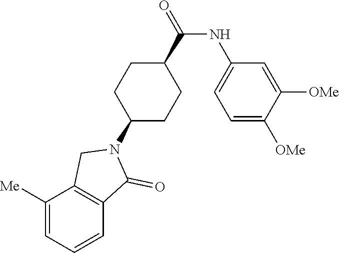 [SMILES: COc1ccc(NC(=O)[C@H]2CC[C@@H](N3Cc4c(C)cccc4C3=O)CC2)cc1OC]

### (1s,4s)-N-(3,4-dimethoxyphenyl)-4-(4-methyl-1-oxoisoindolin-2-yl)cyclohexanecarboxamide

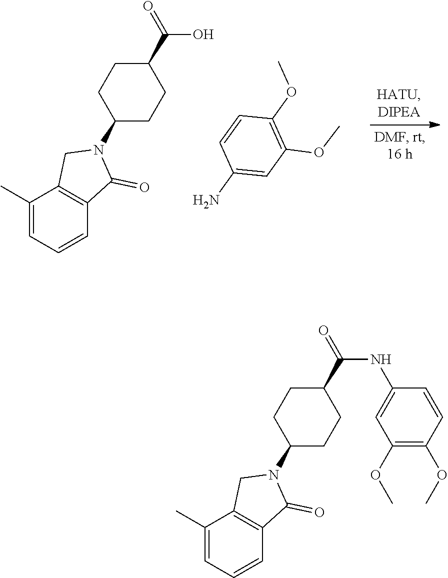 [SMILES: Cc1cccc2c1CN([C@H]1CC[C@@H](C(=O)O)CC1)C2=O ; COc1ccc(N)cc1OC ; COc1ccc(NC(=O)[C@H]2CC[C@@H](N3Cc4c(C)cccc4C3=O)CC2)cc1OC]

### Step 1

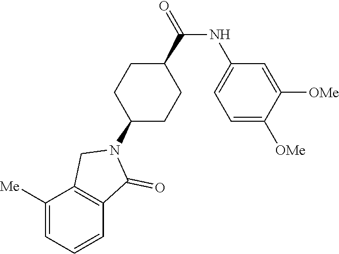 [SMILES: COc1ccc(NC(=O)[C@H]2CC[C@@H](N3Cc4c(C)cccc4C3=O)CC2)cc1OC]

### (1s,4s)-N-(3,4-Dimethoxyphenyl)-4-(4-methyl-1-oxoisoindolin-2-yl)cyclohexanecarboxamide

Following the synthetic procedure for Example 215, but starting with 5-amino-2-methylbenzonitrile gave the title compound as a white solid. LC/MS 409.3 [M+H]⁺. ¹H NMR (400 MHz, CDCl₃) δ 7.67 (d, J=7.4 Hz, 1H), 7.39-7.28 (m, 3H), 7.21 (b, 1H), 6.93-6.89 (m, 1H), 6.83 (d, J=8.6 Hz, 1H), 4.38-4.34 (m, 1H), 4.29 (s, 2H), 3.91 (s, 3H), 3.87 (s, 3H), 2.68-2.66 (m, 1H), 2.33 (s, 3H), 2.27-2.24 (m, 2H), 2.13-1.98 (m, 2H), 1.94-1.75 (m, 4H).

### Example 220

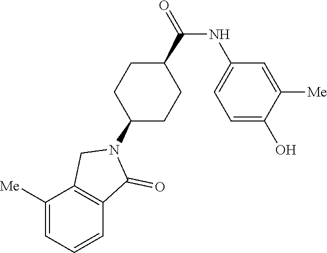 [SMILES: Cc1cc(NC(=O)[C@H]2CC[C@@H](N3Cc4c(C)cccc4C3=O)CC2)ccc1O]

### (1s,4s)-N-(4-Hydroxy-3-methylphenyl)-4-(4-methyl-1-oxoisoindolin-2-yl)cyclohexanecarboxamide

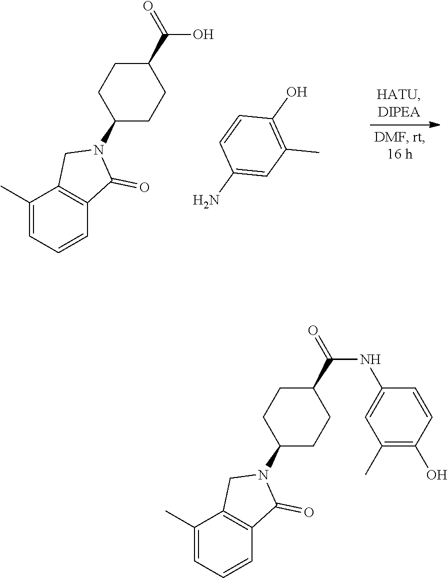

### Step 1

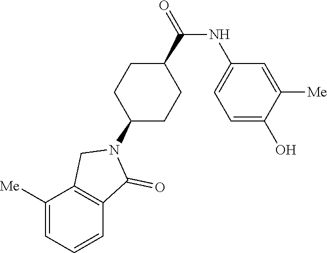 [SMILES: Cc1cc(NC(=O)[C@H]2CC[C@@H](N3Cc4c(C)cccc4C3=O)CC2)ccc1O]

### (1s,4s)-N-(4-Hydroxy-3-methylphenyl)-4-(4-methyl-1-oxoisoindolin-2-yl)cyclohexanecarboxamide

Following the synthetic procedure for Example 215, but starting with 4-amino-2-methylphenol gave the title compound as a white solid. LCMS ESI m/z: 378.9 [M+H]⁺. ¹H NMR (400 MHz, DMSO-d₆) δ 9.44 (s, 1H), 8.99 (s, 1H), 7.50-7.45 (m, 1H), 7.40-7.29 (m, 3H), 7.25-7.15 (m, 1H), 6.68 (d, J=8.8 Hz, 1H), 4.37 (s, 2H), 4.15-4.0 (m, 1H), 2.64-2.62 (m, 1H), 2.34 (s, 3H), 2.17-1.93 (m, 7H), 1.76-1.50 (m, 4H).

### Example 221

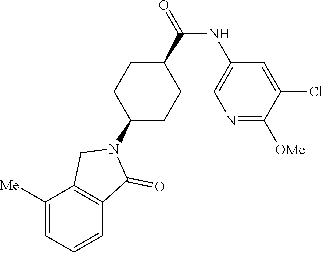 [SMILES: COc1ncc(NC(=O)[C@H]2CC[C@@H](N3Cc4c(C)cccc4C3=O)CC2)cc1Cl]

### (1s,4s)-N-(5-Chloro-6-methoxypyridin-3-yl)-4-(4-methyl-1-oxoisoindolin-2-yl)cyclohexanecarboxamide

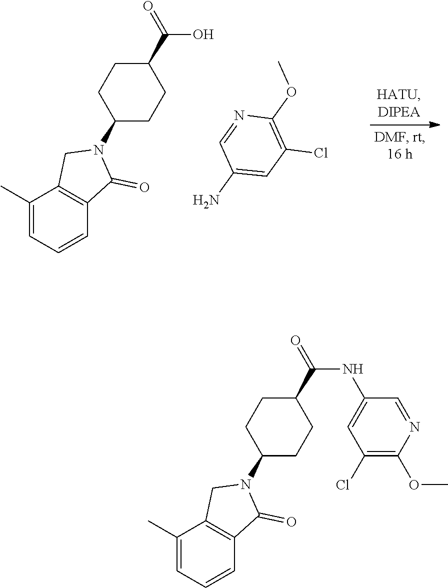

### Step 1

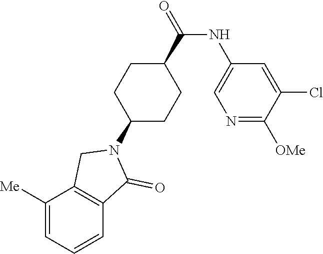 [SMILES: COc1ncc(NC(=O)[C@H]2CC[C@@H](N3Cc4c(C)cccc4C3=O)CC2)cc1Cl]

### (1s,4s)-N-(5-Chloro-6-methoxypyridin-3-yl)-4-(4-methyl-1-oxoisoindolin-2-yl)cyclohexanecarboxamide

Following the synthetic procedure for Example 215, but starting with 5-chloro-6-methoxypyridin-3-amine gave the title compound as a white solid. LCMS ESI m/z: 413.8 [M+H]⁺. ¹H NMR (400 MHz, MeOD-d₄) δ 8.22 (d, J=2.4 Hz, 1H), 8.13 (d, J=2.4 Hz, 1H), 7.63-7.53 (m, 1H), 7.39 (d, J=4.8 Hz, 2H), 4.47 (s, 2H), 4.30-4.26 (m, 1H), 3.97 (s, 3H), 2.76-2.74 (m, 1H), 2.39 (s, 3H), 2.28-2.06 (m, 4H), 1.93-1.72 (m, 4H).

### Example 222

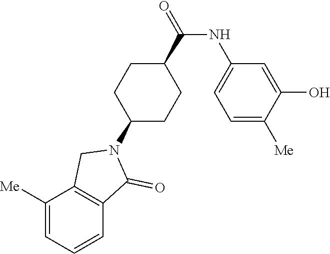 [SMILES: Cc1ccc(NC(=O)[C@H]2CC[C@@H](N3Cc4c(C)cccc4C3=O)CC2)cc1O]

### (1s,4s)-N-(3-Hydroxy-4-methylphenyl)-4-(4-methyl-1-oxoisoindolin-2-yl)cyclohexanecarboxamide

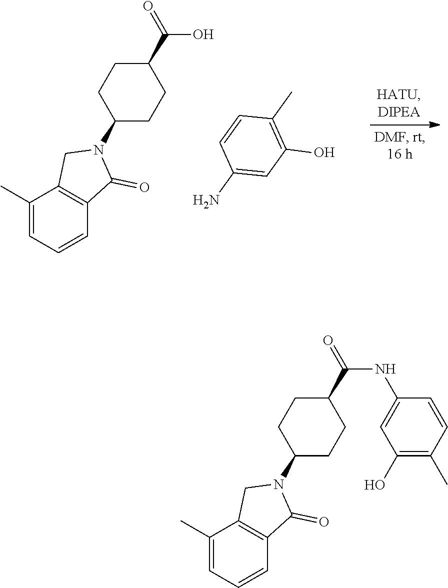

### Step 1

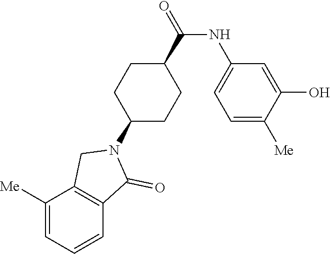 [SMILES: Cc1ccc(NC(=O)[C@H]2CC[C@@H](N3Cc4c(C)cccc4C3=O)CC2)cc1O]

### (1s,4s)-N-(3-Hydroxy-4-methylphenyl)-4-(4-methyl-1-oxoisoindolin-2-yl)cyclohexanecarboxamide

Following the synthetic procedure for Example 215, but starting with 5-amino-2-methylphenol gave the title compound as a white solid. LC/MS ESI 379.2 [M+H]⁺. ¹H NMR (400 MHz, DMSO-d₆) δ 9.57 (s, 1H), 9.19 (s, 1H), 7.51-7.43 (m, 1H), 7.38-7.34 (m, 2H), 7.28 (d, J=1.7 Hz, 1H), 6.90-6.86 (m, 2H), 4.35 (s, 2H), 4.12-4.08 (m, 1H), 2.66-2.64 (m, 1H), 2.32 (s, 3H), 2.14-1.92 (m, 7H), 1.76-1.52 (m, 4H).

### Example 223

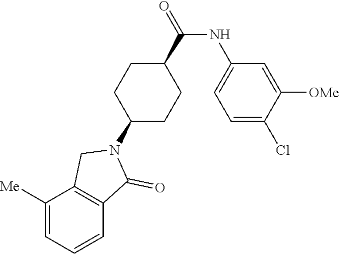 [SMILES: COc1cc(NC(=O)[C@H]2CC[C@@H](N3Cc4c(C)cccc4C3=O)CC2)ccc1Cl]

### (1s,4s)-N-(4-Chloro-3-methoxyphenyl)-4-(4-methyl-1-oxoisoindolin-2-yl)cyclohexanecarboxamide

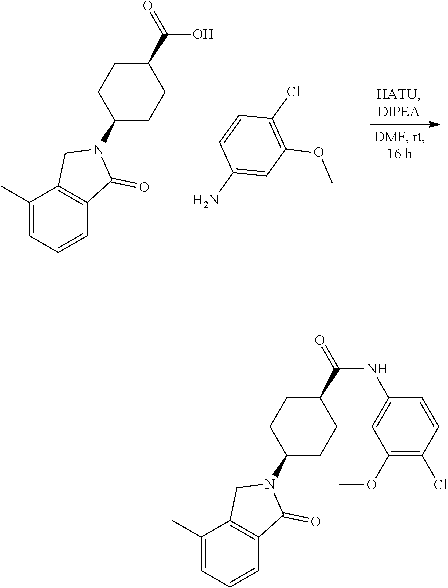

### Step 1

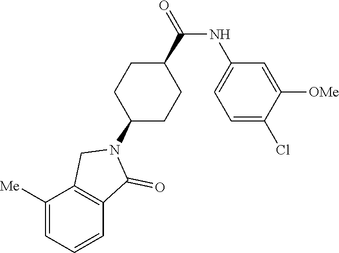 [SMILES: COc1cc(NC(=O)[C@H]2CC[C@@H](N3Cc4c(C)cccc4C3=O)CC2)ccc1Cl]

### (1s,4s)-N-(4-chloro-3-methoxyphenyl)-4-(4-methyl-1-oxoisoindolin-2-yl)cyclohexanecarboxamide

Following the synthetic procedure for Example 215, but starting with 4-chloro-3-methoxyaniline gave the title compound as a white solid. LC/MS ESI 392.9 [M+H]⁺. ¹H NMR (400 MHz, DMSO-d₆) δ 9.92 (s, 1H), 7.55 (d, J=2.2 Hz, 1H), 7.51-7.46 (m, 1H), 7.39-7.35 (m, 2H), 7.32 (d, J=8.6 Hz, 1H), 7.25 (dd, J=8.6, 2.2 Hz, 1H), 4.38 (s, 2H), 4.14-4.10 (m, 1H), 3.83 (s, 3H), 2.71-2.69 (m, 1H), 2.34 (s, 3H), 2.16-2.12 (m, 2H), 2.02-1.96 (m, 2H), 1.76-1.73 (m, 2H), 1.63-1.59 (m, 2H).

### Example 224

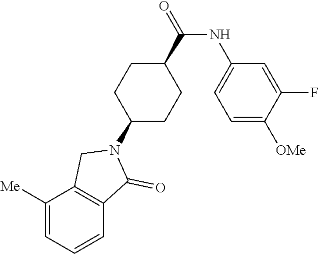 [SMILES: COc1ccc(NC(=O)[C@H]2CC[C@@H](N3Cc4c(C)cccc4C3=O)CC2)cc1F]

### (1s,4s)-N-(3-Fluoro-4-methoxyphenyl)-4-(4-methyl-1-oxoisoindolin-2-yl)cyclohexanecarboxamide

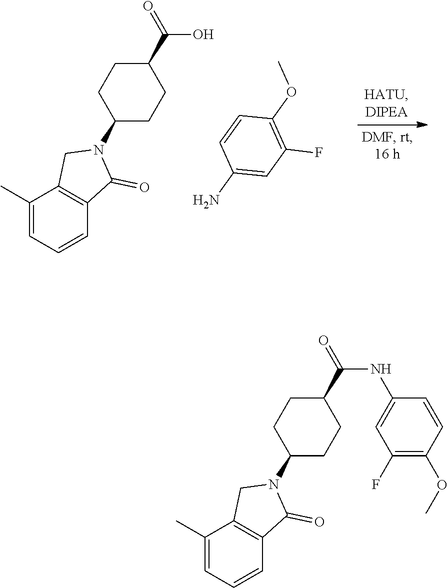

### Step 1

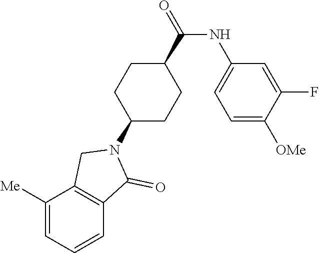 [SMILES: COc1ccc(NC(=O)[C@H]2CC[C@@H](N3Cc4c(C)cccc4C3=O)CC2)cc1F]

### (1s,4s)-N-(3-Fluoro-4-methoxyphenyl)-4-(4-methyl-1-oxoisoindolin-2-yl)cyclohexanecarboxamide

Following the synthetic procedure for Example 215, but starting with 3-fluoro-4-methoxyaniline gave the title compound as a white solid. LC/MS ESI 392.9 [M+H]⁺. ¹H NMR (400 MHz, DMSO-d₆) δ 9.81 (s, 1H), 7.64 (dd, J=13.9, 2.4 Hz, 1H), 7.50-7.46 (m, 1H), 7.40-7.36 (m, 2H), 7.31-7.28 (m, 1H), 7.10 (t, J=9.3 Hz, 1H), 4.38 (s, 2H), 4.16-4.06 (m, 1H), 3.80 (s, 3H), 2.67-2.65 (m, 1H), 2.34 (s, 3H), 2.15-2.11 (m, 2H), 2.02-1.97 (m, 2H), 1.76-1.71 (m, 2H), 1.64-1.57 (m, 2H).

### Example 225

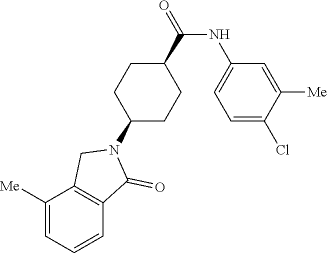 [SMILES: Cc1cc(NC(=O)[C@H]2CC[C@@H](N3Cc4c(C)cccc4C3=O)CC2)ccc1Cl]

### (1s,4s)-N-(4-Chloro-3-methylphenyl)-4-(4-methyl-1-oxoisoindolin-2-yl)cyclohexanecarboxamide

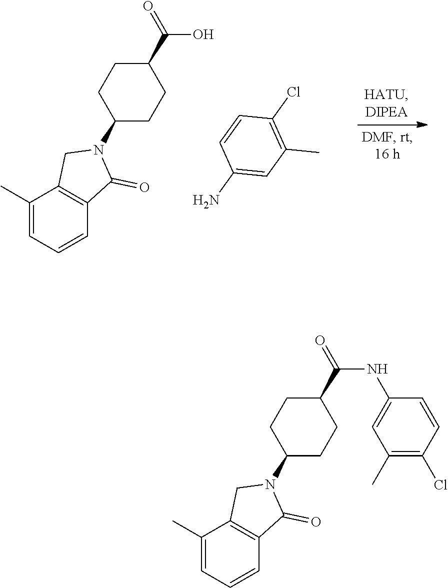

### Step 1

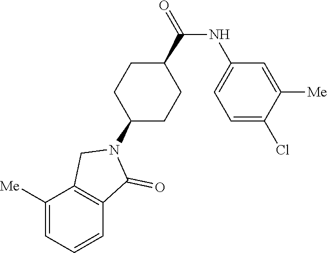 [SMILES: Cc1cc(NC(=O)[C@H]2CC[C@@H](N3Cc4c(C)cccc4C3=O)CC2)ccc1Cl]

### (1s,4s)-N-(4-Chloro-3-methylphenyl)-4-(4-methyl-1-oxoisoindolin-2-yl)cyclohexanecarboxamide

Following the synthetic procedure for Example 215, but starting with 4-chloro-3-methylaniline gave the title compound as a white solid. LC/MS ESI 396.8 [M+H]⁺. ¹H NMR (400 MHz, DMSO-d₆) δ 9.85 (s, 1H), 7.67 (d, J=2.3 Hz, 1H), 7.49-7.45 (m, 2H), 7.39-7.36 (m, 2H), 7.32 (d, J=8.7 Hz, 1H), 4.38 (s, 2H), 4.13-4.09 (m, 1H), 2.70-2.68 (m, 1H), 2.34 (s, 3H), 2.30 (s, 3H), 2.14-2.11 (m, 2H), 2.03-1.93 (m, 2H), 1.76-1.72 (m, 2H), 1.64-1.57 (m, 2H).

### Example 226

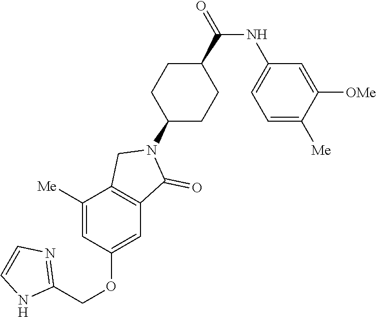 [SMILES: COc1cc(NC(=O)[C@H]2CC[C@@H](N3Cc4c(C)cc(OCc5ncc[nH]5)cc4C3=O)CC2)ccc1C]

### (1s,4s)-4-(6-((1H-Imidazol-2-yl)methoxy)-4-methyl-1-oxoisoindolin-2-yl)-N-(3-methoxy-4-methylphenyl)cyclohexanecarboxamide

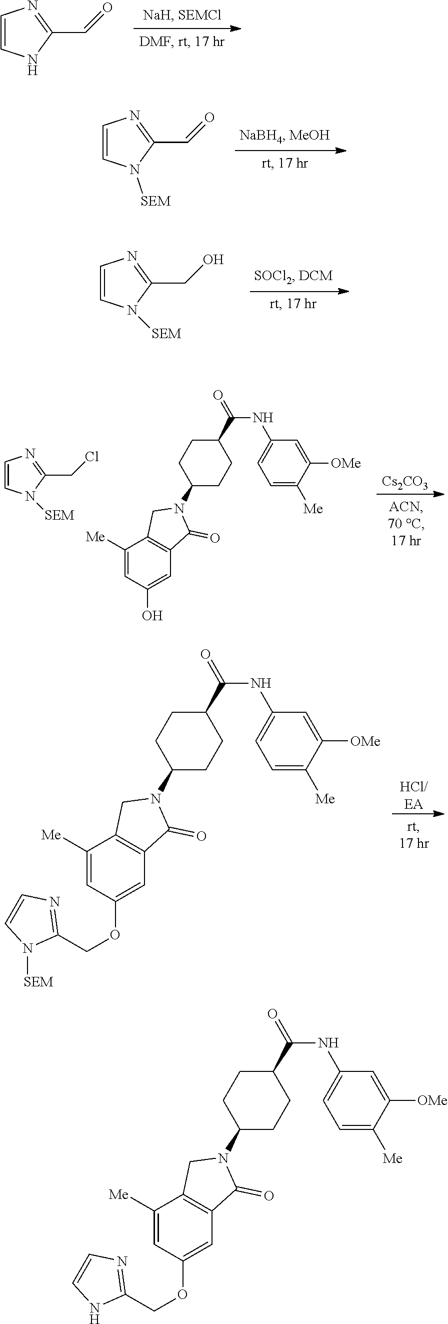

### Step 1

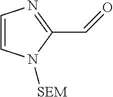 [SMILES: C[Si](C)(C)CCOCn1ccnc1C=O]

### 1-((2-(Trimethylsilyl)ethoxy)methyl)-1H-imidazole-2-carbaldehyde

To a solution of 1H-imidazole-2-carbaldehyde (300 mg, 3.12 mmol) in DMF (2.7 mL) was added NaH (150 mg, 3.78 mmol, 60% wt in mineral oil) and 2-(trimethylsilyl)ethoxy methyl chloride (570 mg, 3.4 mmol, 610 μL). The mixture was stirred at 25° C. for 17 hours. The mixture was quenched with sat. NH₄Cl aq. (30 mL) and extracted with EtOAc (30 mL). The organic phase was concentrated in vacuum to give 1-(2-trimethylsilylethoxymethyl) imidazole-2-carbaldehyde (500 mg, 2.2 mmol, 71% yield) as a yellow oil. ¹HNMR (400 MHz, DMSO-d₆) δ 9.80 (d, J=0.8 Hz, 1H), 7.84 (s, 1H), 7.39 (d, J=0.8 Hz, 1H), 5.76 (s, 2H), 3.57 (t, J=8.0 Hz, 2H), 0.90 (t, J=8.0 Hz, 2H), 0.01 (s, 9H).

### Step 2

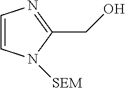 [SMILES: C[Si](C)(C)CCOCn1ccnc1CO]

### 1-((2-(Trimethylsilyl)ethoxy)methyl)-1H-imidazol-2-yl)methanol

To a solution of 1-(2-trimethylsilylethoxymethyl) imidazole-2-carbaldehyde (600 mg, 2.7 mmol) in MeOH (5 mL) was added NaBH₄ (300 mg, 8.0 mmol). The mixture was stirred at 25° C. for 17 hours. The mixture was quenched with water (20 mL) and extracted with EtOAc (20 mL*3). The organic phases were concentrated in vacuo to give (1-((2-(trimethylsilyl)ethoxy)methyl)-1H-imidazol-2-yl)methanol (200 mg, 880 μmol, 33% yield) as yellow oil. ¹HNMR (400 MHz, DMSO-d₆) δ 7.22 (d, J=1.2 Hz, 1H), 6.83 (d, J=1.2 Hz, 1H), 5.37 (s, 2H), 4.51 (s, 2H), 3.51-3.46 (m, 2H), 0.87-0.81 (m, 2H), −0.05 (s, 9H).

### Step 3

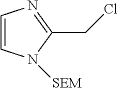 [SMILES: C[Si](C)(C)CCOCn1ccnc1CCl]

### 2-(Chloromethyl)-1-((2-(trimethylsilyl)ethoxy)methyl)-1H-imidazole

A solution of [1-(2-trimethylsilylethoxymethyl) imidazol-2-yl]methanol (360 mg, 1.6 mmol) in DCM (10 mL) was stirred at 25° C. for 17 hours. The mixture was concentrated in vacuo to give 2-(chloromethyl)-1-((2-(trimethylsilyl)ethoxy)methyl)-1H-imidazole, which was used directly in the next step without further purification.

### Step 4

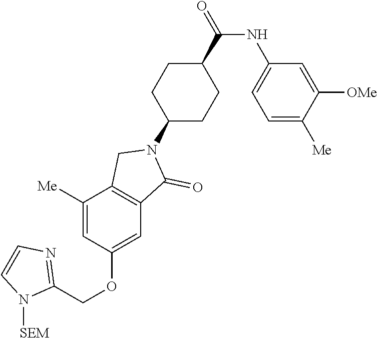 [SMILES: COc1cc(NC(=O)[C@H]2CC[C@@H](N3Cc4c(C)cc(OCc5nccn5COCC[Si](C)(C)C)cc4C3=O)CC2)ccc1C]

### (1s,4s)-N-(3-Methoxy-4-methylphenyl)-4-(4-methyl-1-oxo-6-((1-((2-(trimethylsilyl)ethoxy)methyl)-1H-imidazol-2-yl)methoxy)isoindolin-2-yl)cyclohexane-1-carboxamide

To a solution of 2-[[2-(chloromethyl) imidazol-1-yl]methoxy]ethyl-trimethyl-silane (60 mg, 240 μmol) in MeCN (10 mL) was added Cs₂CO₃ (160 mg, 490 μmol) and (1s,4s)-N-(3-methoxy-4-methylphenyl)-4-(4-methyl-1-oxo-6-((1-((2-(trimethylsilyl)ethoxy)methyl)-1H-imidazol-2-yl)methoxy)isoindolin-2-yl)cyclohexane-1-carboxamide (99 mg, 240 μmol). The reaction was stirred at 70° C. for 17 hours. The mixture was poured into water (20 mL) and extracted with EtOAc (20 mL*3). The organic layers were dried and concentrated in vacuum to give (1s,4s)-N-(3-methoxy-4-methylphenyl)-4-(4-methyl-1-oxo-6-((1-((2-(trimethylsilyl)ethoxy)methyl)-1H-imidazol-2-yl)methoxy)isoindolin-2-yl)cyclohexanecarboxamide (70 mg, 110 μmol, 47% yield) as a white solid. LCMS ESI m/z 618.8 [M+H]⁺.

### Step 5

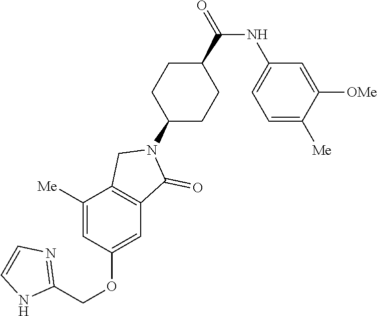 [SMILES: COc1cc(NC(=O)[C@H]2CC[C@@H](N3Cc4c(C)cc(OCc5ncc[nH]5)cc4C3=O)CC2)ccc1C]

### (1s,4s)-4-(6-((1H-Imidazol-2-yl)methoxy)-4-methyl-1-oxoisoindolin-2-yl)-N-(3-methoxy-4-methylphenyl)cyclohexane-1-carboxamide

A solution of (1s,4s)-N-(3-methoxy-4-methylphenyl)-4-(4-methyl-1-oxo-6-((1-((2-(trimethylsilyl)ethoxy)methyl)-1H-imidazol-2-yl)methoxy)isoindolin-2-yl)cyclohexanecarboxamide (70 mg, 110 μmol) in HCl/EtOAc (5 mL) was stirred at 25° C. for 17 hours. The mixture was concentrated and purified by prep-HPLC to give (1s,4s)-4-(6-((1H-imidazol-2-yl)methoxy)-4-methyl-1-oxoisoindolin-2-yl)-N-(3-methoxy-4-methylphenyl)cyclohexanecarboxamide (15 mg, 30 μmol, 27% yield) as a pink solid. LCMS ESI m/z 488.9 [M+H]⁺. ¹HNMR (400 MHz, DMSO-d₆) δ 12.28 (br, 1H), 9.70 (s, 1H), 7.32 (d, J=1.7 Hz, 1H), 7.18-7.09 (m, 3H), 7.03 (dd, J=8.2, 4.9 Hz, 2H), 6.90 (br, 1H), 5.08 (s, 2H), 4.30 (s, 2H), 4.17-4.04 (m, 1H), 3.76 (s, 3H), 2.68-2.66 (m, 1H), 2.29 (s, 3H), 2.18-2.06 (m, 5H), 2.03-1.96 (m, 2H), 1.76-1.71 (m, 2H), 1.64-1.58 (m, 2H).

### Example 227

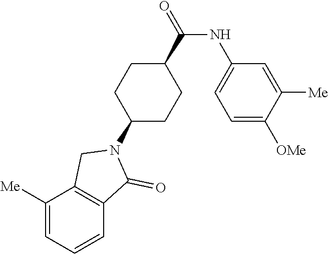 [SMILES: COc1ccc(NC(=O)[C@H]2CC[C@@H](N3Cc4c(C)cccc4C3=O)CC2)cc1C]

### (1s,4s)-N-(4-Methoxy-3-methylphenyl)-4-(4-methyl-1-oxoisoindolin-2-yl)cyclohexanecarboxamide

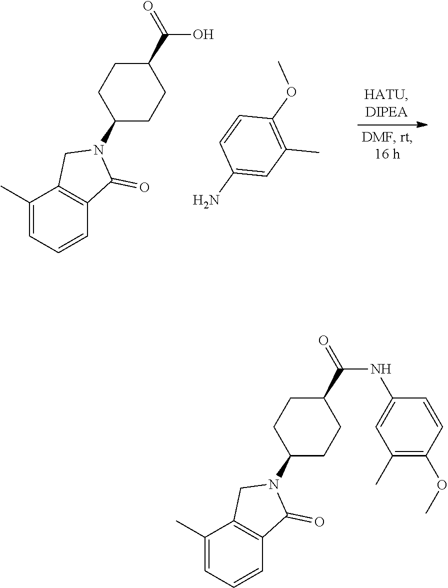

### Step 1

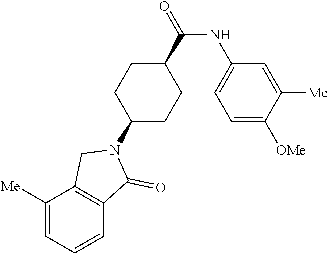 [SMILES: COc1ccc(NC(=O)[C@H]2CC[C@@H](N3Cc4c(C)cccc4C3=O)CC2)cc1C]

### (1s,4s)-N-(4-Methoxy-3-methylphenyl)-4-(4-methyl-1-oxoisoindolin-2-yl)cyclohexanecarboxamide

Following the synthetic procedure for Example 215, but starting with 4-methoxy-3-methylaniline gave the title compound as a white solid. LC/MS ESI 392.9 [M+H]⁺. ¹H NMR (400 MHz, DMSO-d₆) 09.56 (s, 1H), 7.50-7.47 (m, 1H), 7.43-7.41 (m, 1H), 7.41-7.35 (m, 3H), 6.85 (d, J=8.8 Hz, 1H), 4.38 (s, 2H), 4.14-4.11 (m, 1H), 3.75 (s, 3H), 2.66-2.64 (m, 1H), 2.34 (s, 3H), 2.13 (s, 3H), 2.12-1.95 (m, 4H), 1.78-1.55 (m, 4H).

### Example 228

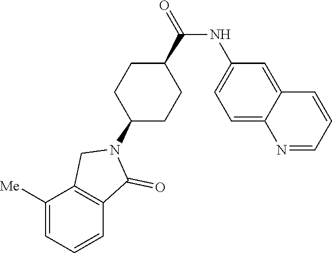 [SMILES: Cc1cccc2c1CN([C@H]1CC[C@@H](C(=O)Nc3ccc4ncccc4c3)CC1)C2=O]

### (1s,4s)-4-(4-Methyl-1-oxoisoindolin-2-yl)-N-(quinolin-6-yl)cyclohexanecarboxamide

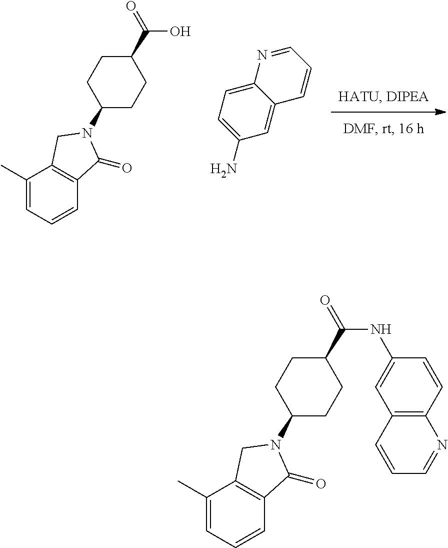 [SMILES: Cc1cccc2c1CN([C@H]1CC[C@@H](C(=O)O)CC1)C2=O ; Nc1ccc2ncccc2c1 ; Cc1cccc2c1CN([C@H]1CC[C@@H](C(=O)Nc3ccc4ncccc4c3)CC1)C2=O]

### Step 1

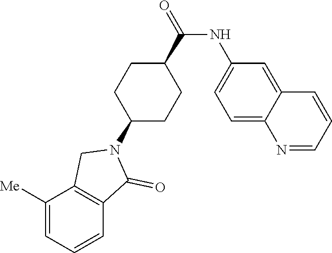 [SMILES: Cc1cccc2c1CN([C@H]1CC[C@@H](C(=O)Nc3ccc4ncccc4c3)CC1)C2=O]

### (1s,4s)-4-(4-Methyl-1-oxoisoindolin-2-yl)-N-(quinolin-6-yl)cyclohexanecarboxamide

Following the synthetic procedure for Example 215, but starting with quinolin-6-amine gave the title compound as a yellow solid. LC/MS ESI 399.9 [M+H]⁺. ¹H NMR (400 MHz, DMSO-d₆) δ 10.16 (s, 1H), 8.78 (dd, J=4.2, 1.6 Hz, 1H), 8.48 (d, J=2.2 Hz, 1H), 8.29 (d, J=7.7 Hz, 1H), 7.96 (d, J=9.1 Hz, 1H), 7.83 (dd, J=9.1, 2.3 Hz, 1H), 7.51-7.45 (m, 2H), 7.40-7.35 (m, 2H), 4.40 (s, 2H), 4.20-4.11 (m, 1H), 2.81-2.79 (s, 1H), 2.35 (s, 3H), 2.20-2.17 (m, 2H), 2.11-2.01 (m, 2H), 1.81-1.78 (m, 2H), 1.69-1.60 (m, 2H).

### Example 229

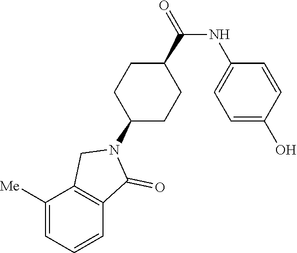 [SMILES: Cc1cccc2c1CN([C@H]1CC[C@@H](C(=O)Nc3ccc(O)cc3)CC1)C2=O]

### (1s,4s)-N-(4-Hydroxyphenyl)-4-(4-methyl-1-oxoisoindolin-2-yl)cyclohexanecarboxamide

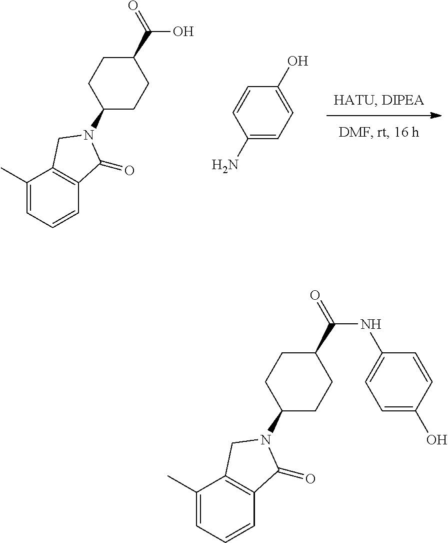 [SMILES: Cc1cccc2c1CN([C@H]1CC[C@@H](C(=O)O)CC1)C2=O ; Nc1ccc(O)cc1 ; Cc1cccc2c1CN([C@H]1CC[C@@H](C(=O)Nc3ccc(O)cc3)CC1)C2=O]

### Step 1

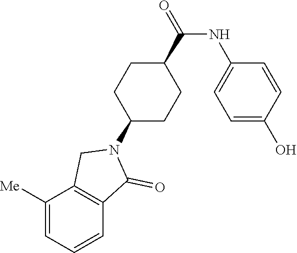 [SMILES: Cc1cccc2c1CN([C@H]1CC[C@@H](C(=O)Nc3ccc(O)cc3)CC1)C2=O]

### (1s,4s)-N-(4-Hydroxyphenyl)-4-(4-methyl-1-oxoisoindolin-2-yl)cyclohexanecarboxamide

Following the synthetic procedure for Example 215, but starting with 4-aminophenol gave the title compound as a brown solid. LC/MS ESI 364.8 [M+H]⁺. ¹H NMR (400 MHz, DMSO-d₆) δ 9.49 (s, 1H), 9.09 (s, 1H), 7.50-7.42 (m, 1H), 7.41-7.31 (m, 4H), 6.70-6.61 (m, 2H), 4.35 (s, 2H), 4.15-4.03 (m, 1H), 2.62-2.60 (m, 1H), 2.32 (s, 3H), 2.17-1.88 (m, 4H), 1.77-1.51 (m, 4H).

### Example 230

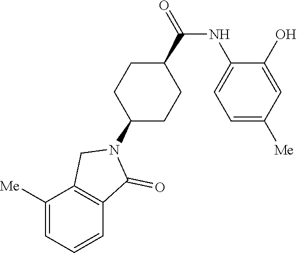 [SMILES: Cc1ccc(NC(=O)[C@H]2CC[C@@H](N3Cc4c(C)cccc4C3=O)CC2)c(O)c1]

### (1s,4s)-N-(2-Hydroxy-4-methylphenyl)-4-(4-methyl-1-oxoisoindolin-2-yl)cyclohexanecarboxamide

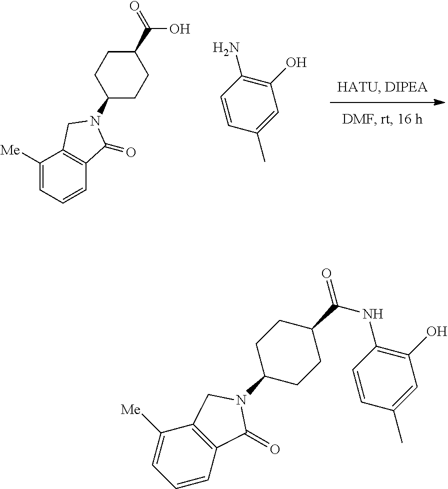

### Step 1

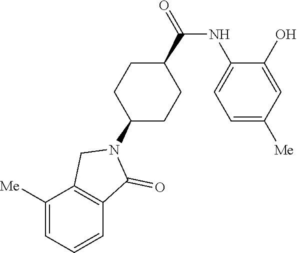 [SMILES: Cc1ccc(NC(=O)[C@H]2CC[C@@H](N3Cc4c(C)cccc4C3=O)CC2)c(O)c1]

### (1s,4s)-N-(2-Hydroxy-4-methylphenyl)-4-(4-methyl-1-oxoisoindolin-2-yl)cyclohexanecarboxamide

Following the synthetic procedure for Example 215, but starting with 2-amino-5-methylphenol gave the title compound as a white solid. LC/MS ESI 378.9 [M+H]⁺. ¹H NMR (400 MHz, DMSO-d₆) δ 9.55 (br, 1H), 9.09 (s, 1H), 7.59-7.43 (m, 2H), 7.42-7.29 (m, 2H), 6.68 (d, J=1.3 Hz, 1H), 6.59 (dd, J=8.1, 1.3 Hz, 1H), 4.36 (s, 2H), 4.17-4.05 (m, 1H), 2.83-2.81 (m, 1H), 2.33 (s, 3H), 2.20 (s, 3H), 2.15-2.10 (m, 2H), 2.05-1.91 (m, 2H), 1.76-1.70 (m, 2H), 1.64-1.58 (m, 2H).

### Example 231

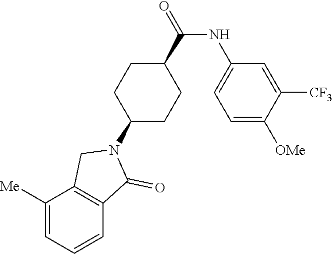 [SMILES: COc1ccc(NC(=O)[C@H]2CC[C@@H](N3Cc4c(C)cccc4C3=O)CC2)cc1C(F)(F)F]

### (1s,4s)-N-(4-Methoxy-3-(trifluoromethyl)phenyl)-4-(4-methyl-1-oxoisoindolin-2-yl)cyclohexanecarboxamide

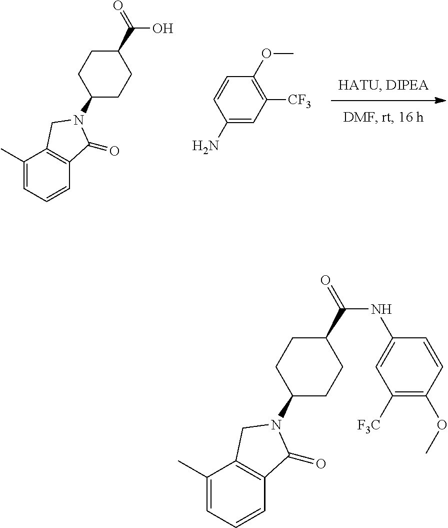 [SMILES: Cc1cccc2c1CN([C@H]1CC[C@@H](C(=O)O)CC1)C2=O ; COc1ccc(N)cc1C(F)(F)F ; COc1ccc(NC(=O)[C@H]2CC[C@@H](N3Cc4c(C)cccc4C3=O)CC2)cc1C(F)(F)F]

### Step 1

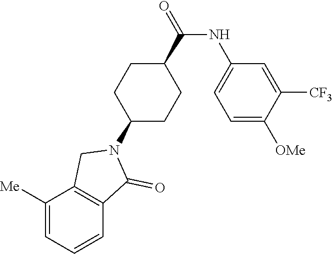 [SMILES: COc1ccc(NC(=O)[C@H]2CC[C@@H](N3Cc4c(C)cccc4C3=O)CC2)cc1C(F)(F)F]

### (1s,4s)-N-(4-Methoxy-3-(trifluoromethyl)phenyl)-4-(4-methyl-1-oxoisoindolin-2-yl)cyclohexanecarboxamide

Following the synthetic procedure for Example 215, but starting with 4-methoxy-3-(trifluoromethyl) aniline gave the title compound as a white solid. LC/MS ESI 446.8 [M+H]⁺. ¹H NMR (400 MHz, DMSO-d₆) δ 9.91 (s, 1H), 8.02 (d, J=2.6 Hz, 1H), 7.81 (dd, J=9.0, 2.5 Hz, 1H), 7.50-7.47 (m, 1H), 7.70-7.36 (m, 2H), 7.23 (d, J=9.0 Hz, 1H), 4.38 (s, 2H), 4.13-7.11 (m, 1H), 3.85 (s, 3H), 2.68-2.66 (m, 1H), 2.34 (s, 3H), 2.15-2.13 (m, 2H), 2.00-1.96 (m, 2H), 1.76-1.72 (m, 2H), 1.66-1.56 (m, 2H).

### Example 232

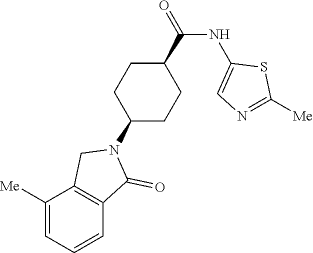 [SMILES: Cc1ncc(NC(=O)[C@H]2CC[C@@H](N3Cc4c(C)cccc4C3=O)CC2)s1]

### (1s,4s)-4-(4-Methyl-1-oxoisoindolin-2-yl)-N-(2-methylthiazol-5-yl)cyclohexanecarboxamide

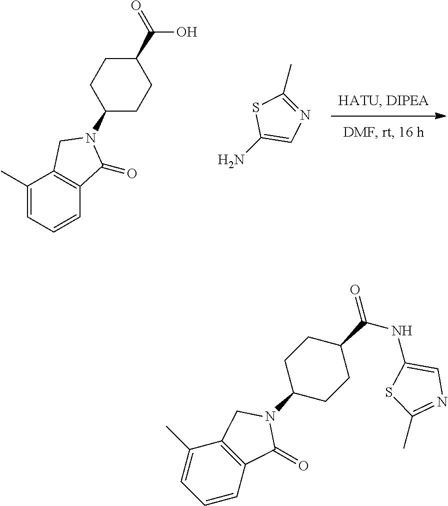

### Step 1

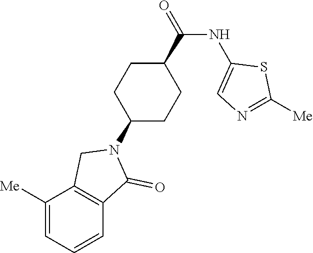 [SMILES: Cc1ncc(NC(=O)[C@H]2CC[C@@H](N3Cc4c(C)cccc4C3=O)CC2)s1]

### (1s,4s)-4-(4-Methyl-1-oxoisoindolin-2-yl)-N-(2-methylthiazol-5-yl)cyclohexanecarboxamide

Following the synthetic procedure for Example 215, but starting with 2-methylthiazol-5-amine gave the title compound as a white solid. LC/MS ESI 369.8 [M+H]⁺. ¹H NMR (400 MHz, DMSO-d₆) 011.05 (s, 1H), 7.49-7.46 (m, 1H), 7.38-7.35 (m, 2H), 7.32 (s, 1H), 4.36 (s, 2H), 4.16-4.05 (m, 1H), 2.74-2.72 (m, 1H), 2.53 (s, 3H), 2.33 (s, 3H), 2.16-2.12 (m, 2H), 1.95-1.85 (m, 2H), 1.77-1.73 (m, 2H), 1.63-1.60 (m, 2H).

### Example 233

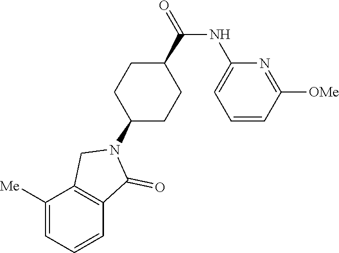 [SMILES: COc1cccc(NC(=O)[C@H]2CC[C@@H](N3Cc4c(C)cccc4C3=O)CC2)n1]

### (1s,4s)-N-(6-Methoxypyridin-2-yl)-4-(4-methyl-1-oxoisoindolin-2-yl)cyclohexanecarboxamide

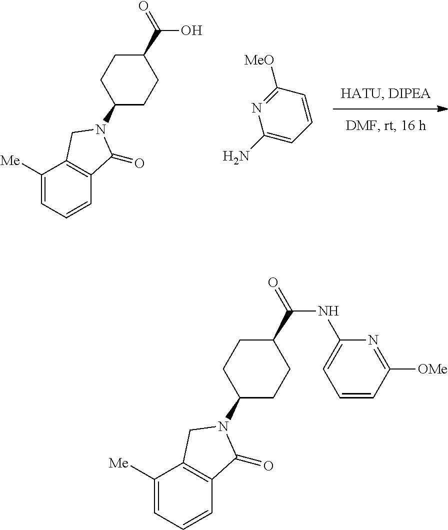 [SMILES: Cc1cccc2c1CN([C@H]1CC[C@@H](C(=O)O)CC1)C2=O ; COc1cccc(N)n1 ; COc1cccc(NC(=O)[C@H]2CC[C@@H](N3Cc4c(C)cccc4C3=O)CC2)n1]

### Step 1

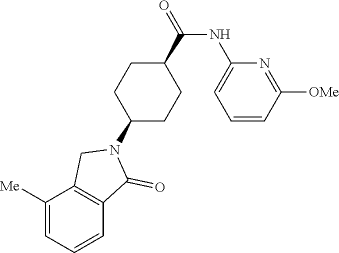 [SMILES: COc1cccc(NC(=O)[C@H]2CC[C@@H](N3Cc4c(C)cccc4C3=O)CC2)n1]

### (1s,4s)-N-(6-Methoxypyridin-2-yl)-4-(4-methyl-1-oxoisoindolin-2-yl)cyclohexanecarboxamide

Following the synthetic procedure for Example 215, but starting with 6-methoxypyridin-2-amine gave the title compound as a white solid. LC/MS ESI 379.9 [M+H]⁺. ¹H NMR (400 MHz, DMSO-d₆) δ 10.14 (s, 1H), 7.78-7.60 (m, 2H), 7.52-7.44 (m, 1H), 7.40-7.32 (m, 2H), 6.50 (d, J=7.6 Hz, 1H), 4.38 (s, 2H), 4.17-4.05 (m, 1H), 3.84 (s, 3H), 2.86-2.84 (m, 1H), 2.34 (s, 3H), 2.16-1.93 (m, 4H), 1.80-1.51 (m, 4H).

### Example 234

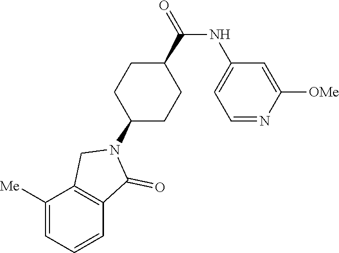 [SMILES: COc1cc(NC(=O)[C@H]2CC[C@@H](N3Cc4c(C)cccc4C3=O)CC2)ccn1]

### (1s,4s)-N-(2-Methoxypyridin-4-yl)-4-(4-methyl-1-oxoisoindolin-2-yl)cyclohexanecarboxamide

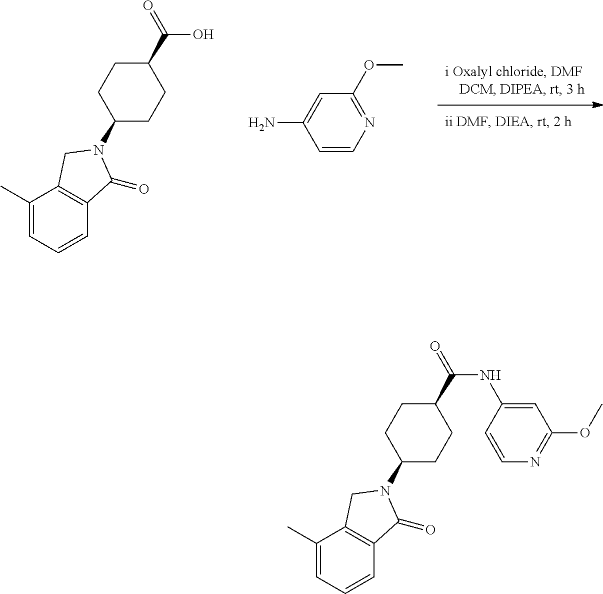

### Step 1

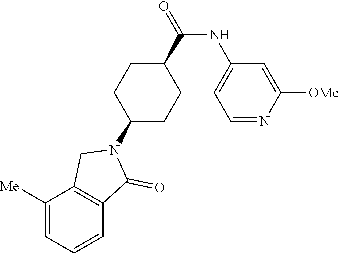 [SMILES: COc1cc(NC(=O)[C@H]2CC[C@@H](N3Cc4c(C)cccc4C3=O)CC2)ccn1]

### (1s,4s)-N-(2-Methoxypyridin-4-yl)-4-(4-methyl-1-oxoisoindolin-2-yl)cyclohexanecarboxamide

To a solution of 4-(4-methyl-1-oxo-isoindolin-2-yl)cyclohexanecarboxylic acid (200 mg, 732 μmol) in DCM (5.0 mL) was added oxalyl chloride (186 mg, 1.46 mmol, 128 μL) and one drop of DMF at 0° C. under nitrogen. The mixture was stirred at 25° C. for 3 hours. The solution was concentrated to give 4-(4-methyl-1-oxo-isoindolin-2-yl)cyclohexanecarbonyl chloride (200 mg, crude) as a yellow oil. To a solution of 4-(4-methyl-1-oxo-isoindolin-2-yl)cyclohexanecarbonyl chloride (200 mg, 685 μmol) and 2-methoxypyridin-4-amine (111 mg, 891 μmol) in DMF (5.0 mL) was added DIPEA (443 mg, 3.43 mmol, 597 μL). The mixture was stirred at 25° C. for 2 hours. The mixture was poured into water and extracted with EtOAc. The organic extracts were washed with brine, dried by Na₂SO₄, filtered and concentrated to give a crude oil. The crude product was purified by silica gel chromatography to give a yellow solid (50 mg). The yellow solid was purified by prep-HPLC to give (1s,4s)-N-(2-methoxypyridin-4-yl)-4-(4-methyl-1-oxoisoindolin-2-yl)cyclohexanecarboxamide (10 mg, 26 μmol, 3.8% yield) as a white solid. LCMS ESI m/z: 379.9 [M+H]⁺. ¹H NMR (400 MHz, DMSO) δ 10.14 (s, 1H), 8.01 (d, J=5.7 Hz, 1H), 7.50-7.46 (m, 1H), 7.40-7.34 (m, 2H), 7.19 (d, J=1.5 Hz, 1H), 7.15 (dd, J=5.7, 1.8 Hz, 1H), 4.38 (s, 2H), 4.13-4.09 (m, 1H), 3.82 (s, 3H), 2.73-2.71 (m, 1H), 2.34 (s, 3H), 2.15-2.09 (m, 2H), 2.00-1.90 (m, 2H), 1.78-1.73 (m, 2H), 1.64-1.58 (m, 2H).

### Example 235

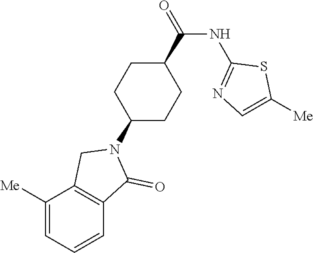 [SMILES: Cc1cnc(NC(=O)[C@H]2CC[C@@H](N3Cc4c(C)cccc4C3=O)CC2)s1]

### (1s,4s)-4-(4-Methyl-1-oxoisoindolin-2-yl)-N-(5-methylthiazol-2-yl)cyclohexanecarboxamide

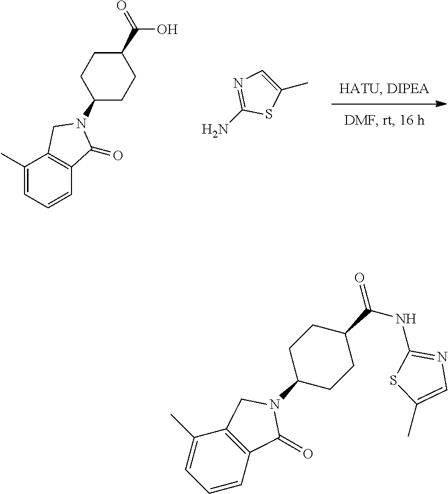

### Step 1

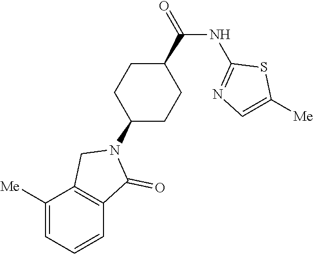 [SMILES: Cc1cnc(NC(=O)[C@H]2CC[C@@H](N3Cc4c(C)cccc4C3=O)CC2)s1]

### (1s,4s)-4-(4-Methyl-1-oxoisoindolin-2-yl)-N-(5-methylthiazol-2-yl)cyclohexanecarboxamide

Following the synthetic procedure for Example 215, but starting with 5-methylthiazol-2-amine gave the title compound as a white solid. LC/MS ESI 369.8 [M+H]⁺. ¹H NMR (400 MHz, DMSO-d₆) δ 11.84 (b, 1H), 7.51-7.44 (m, 1H), 7.40-7.33 (m, 2H), 7.12 (d, J=1.3 Hz, 1H), 4.37 (s, 2H), 4.13-4.09 (m, 1H), 2.82-2.79 (m, 1H), 2.34 (2s, 6H), 2.15-2.06 (m, 2H), 1.98-1.86 (m, 2H), 1.76-1.73 (m, 2H), 1.66-1.58 (m, 2H).

### Example 236

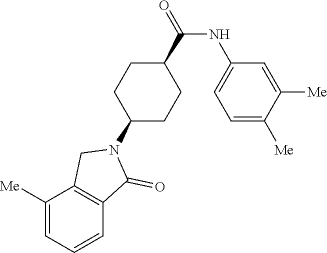 [SMILES: Cc1ccc(NC(=O)[C@H]2CC[C@@H](N3Cc4c(C)cccc4C3=O)CC2)cc1C]

### (1s,4s)-N-(3,4-Dimethylphenyl)-4-(4-methyl-1-oxoisoindolin-2-yl)cyclohexanecarboxamide

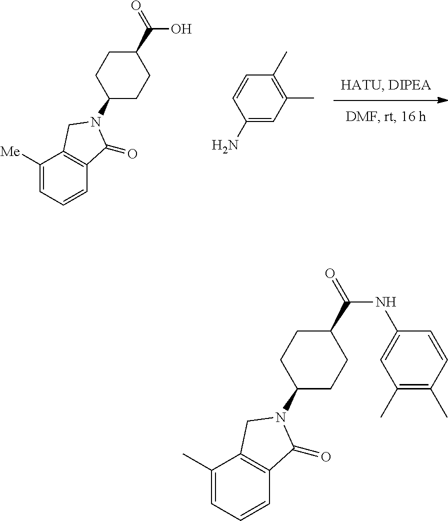

### Step 1

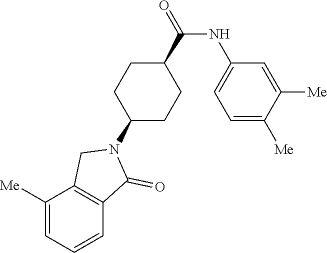 [SMILES: Cc1ccc(NC(=O)[C@H]2CC[C@@H](N3Cc4c(C)cccc4C3=O)CC2)cc1C]

### (1s,4s)-N-(3,4-Dimethylphenyl)-4-(4-methyl-1-oxoisoindolin-2-yl)cyclohexanecarboxamide

Following the synthetic procedure for Example 215, but starting with 3,4-dimethylaniline gave the title compound as a white solid. LC/MS ESI 376.9 [M+H]⁺. ¹H NMR (400 MHz, DMSO-d₆) δ 9.62 (s, 1H), 7.50-7.46 (m, 1H), 7.43 (s, 1H), 7.39-7.37 (m, 2H), 7.35-7.32 (m, 1H), 7.04 (d, J=8.2 Hz, 1H), 4.38 (s, 2H), 4.16-4.07 (m, 1H), 2.68-2.66 (m, 1H), 2.34 (s, 3H), 2.19 (s, 3H), 2.16 (s, 3H), 2.14-1.96 (m, 4H), 1.72-1.59 (m, 4H).

### Example 237

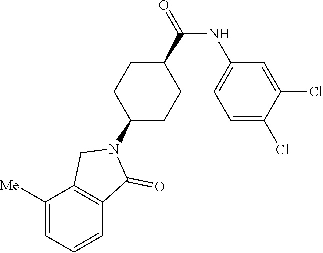 [SMILES: Cc1cccc2c1CN([C@H]1CC[C@@H](C(=O)Nc3ccc(Cl)c(Cl)c3)CC1)C2=O]

### (1s,4s)-N-(3,4-Dichlorophenyl)-4-(4-methyl-1-oxoisoindolin-2-yl)cyclohexanecarboxamide

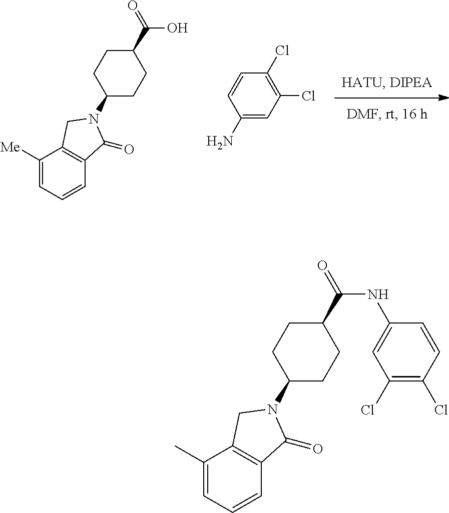 [SMILES: Cc1cccc2c1CN([C@H]1CC[C@@H](C(=O)O)CC1)C2=O ; Nc1ccc(Cl)c(Cl)c1 ; Cc1cccc2c1CN([C@H]1CC[C@@H](C(=O)Nc3ccc(Cl)c(Cl)c3)CC1)C2=O]

### Step 1

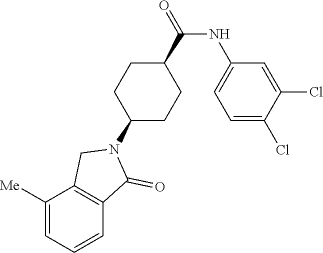 [SMILES: Cc1cccc2c1CN([C@H]1CC[C@@H](C(=O)Nc3ccc(Cl)c(Cl)c3)CC1)C2=O]

### (1s,4s)-N-(3,4-Dichlorophenyl)-4-(4-methyl-1-oxoisoindolin-2-yl)cyclohexanecarboxamide

Following the synthetic procedure for Example 215, but starting with 3,4-dichloroaniline gave the title compound as a white solid. LC/MS ESI 416.7 [M+H]⁺. ¹H NMR (400 MHz, DMSO-d₆) δ 10.11 (s, 1H), 8.08 (d, J=2.0 Hz, 1H), 7.60-7.51 (m, 2H), 7.50-7.44 (m, 1H), 7.42-7.30 (m, 2H), 4.38 (s, 2H), 4.14-4.10 (m, 1H), 2.71-2.69 (m, 1H), 2.34 (s, 3H), 2.16-1.92 (m, 4H), 1.80-1.56 (m, 4H).

### Example 238

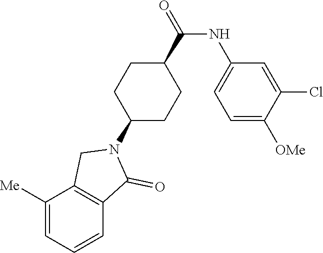 [SMILES: COc1ccc(NC(=O)[C@H]2CC[C@@H](N3Cc4c(C)cccc4C3=O)CC2)cc1Cl]

### (1s,4s)-N-(3-Chloro-4-methoxyphenyl)-4-(4-methyl-1-oxoisoindolin-2-yl)cyclohexanecarboxamide

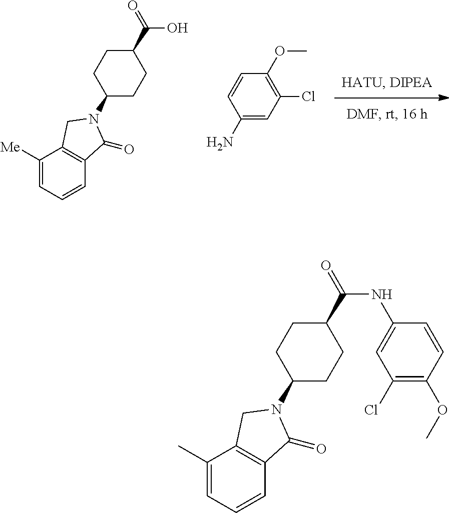

### Step 1

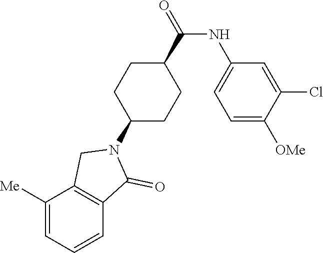 [SMILES: COc1ccc(NC(=O)[C@H]2CC[C@@H](N3Cc4c(C)cccc4C3=O)CC2)cc1Cl]

### (1s,4s)-N-(3-Chloro-4-methoxyphenyl)-4-(4-methyl-1-oxoisoindolin-2-yl)cyclohexanecarboxamide

Following the synthetic procedure for Example 215, but starting with 3-chloro-4-methoxyaniline gave the title compound as a white solid. LC/MS ESI 412.8 [M+H]⁺. ¹H NMR (400 MHz, DMSO-d₆) δ 9.80 (s, 1H), 7.86 (d, J=2.5 Hz, 1H), 7.49-7.44 (m, 2H), 7.39-7.37 (m, 2H), 7.10 (d, J=9.0 Hz, 1H), 4.38 (s, 2H), 4.14-4.09 (m, 1H), 3.82 (s, 3H), 2.68-2.66 (m, 1H), 2.34 (s, 3H), 2.14-1.96 (m, 4H), 1.78-1.56 (m, 4H).

### Example 239

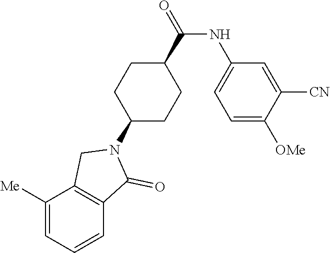 [SMILES: COc1ccc(NC(=O)[C@H]2CC[C@@H](N3Cc4c(C)cccc4C3=O)CC2)cc1C#N]

### (1s,4s)-N-(3-Cyano-4-methoxyphenyl)-4-(4-methyl-1-oxoisoindolin-2-yl)cyclohexanecarboxamide

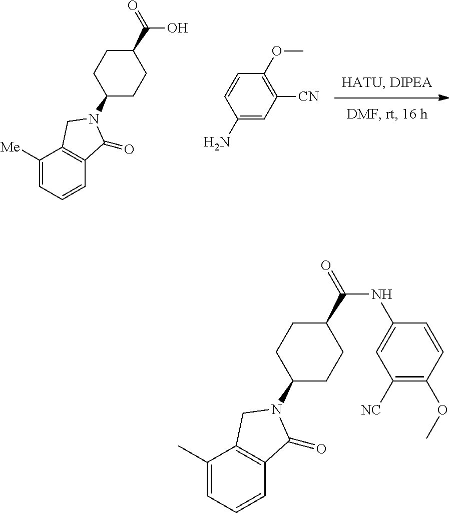 [SMILES: Cc1cccc2c1CN([C@H]1CC[C@@H](C(=O)O)CC1)C2=O ; COc1ccc(N)cc1C#N ; COc1ccc(NC(=O)[C@H]2CC[C@@H](N3Cc4c(C)cccc4C3=O)CC2)cc1C#N]

### Step 1

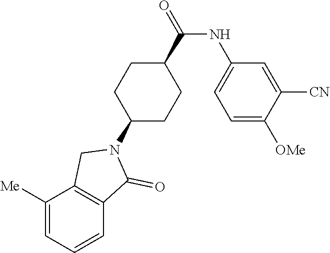 [SMILES: COc1ccc(NC(=O)[C@H]2CC[C@@H](N3Cc4c(C)cccc4C3=O)CC2)cc1C#N]

### (1s,4s)-N-(3-Cyano-4-methoxyphenyl)-4-(4-methyl-1-oxoisoindolin-2-yl)cyclohexanecarboxamide

Following the synthetic procedure for Example 215, but starting with 5-amino-2-methoxybenzonitrile gave the title compound as a white solid. LC/MS ESI 403.9 [M+H]⁺. ¹H NMR (400 MHz, DMSO-d₆) δ9.95 (s, 1H), 8.04 (d, J=2.6 Hz, 1H), 7.78 (dd, J=9.2, 2.6 Hz, 1H), 7.51-7.45 (m, 1H), 7.41-7.35 (m, 2H), 7.23 (d, J=9.2 Hz, 1H), 4.38 (s, 2H), 4.15-4.11 (m, 1H), 3.88 (s, 3H), 2.69-2.67 (m, 1H), 2.34 (s, 3H), 2.15-2.13 (m, 2H), 2.01-1.96 (m, 2H), 1.76-1.72 (m, 2H), 1.64-1.60 (m, 2H).

### Example 240

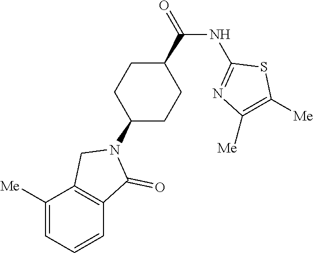 [SMILES: Cc1cccc2c1CN([C@H]1CC[C@@H](C(=O)Nc3nc(C)c(C)s3)CC1)C2=O]

### (1s,4s)-N-(4,5-Dimethylthiazol-2-yl)-4-(4-methyl-1-oxoisoindolin-2-yl)cyclohexanecarboxamide

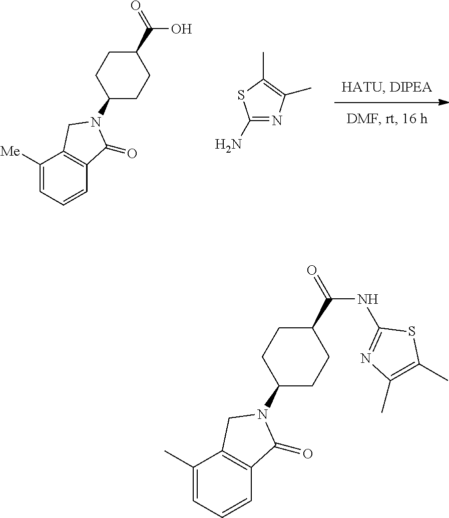

### Step 1

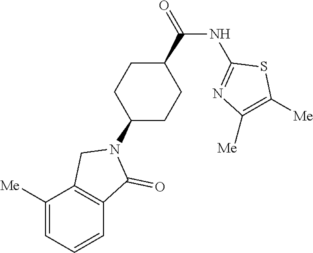 [SMILES: Cc1cccc2c1CN([C@H]1CC[C@@H](C(=O)Nc3nc(C)c(C)s3)CC1)C2=O]

### (1s,4s)-N-(4,5-Dimethylthiazol-2-yl)-4-(4-methyl-1-oxoisoindolin-2-yl)cyclohexanecarboxamide

Following the synthetic procedure for Example 215, but starting with 4,5-dimethylthiazol-2-amine gave the title compound as a white solid. LC/MS ESI 383.8 [M+H]⁺. ¹H NMR (400 MHz, DMSO-d₆) 011.79 (b, 1H), 7.51-7.45 (m, 1H), 7.40-7.36 (m, 2H), 4.37 (s, 2H), 4.14-4.08 (m, 1H), 2.79-2.77 (m, 1H), 2.34 (s, 3H), 2.24 (s, 3H), 2.15 (s, 3H), 2.13-2.11 (m, 2H), 1.94-1.90 (m, 2H), 1.75-1.71 (m, 2H), 1.66-1.55 (m, 2H).

### Example 241

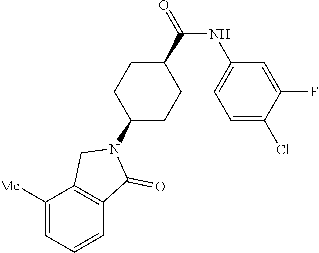 [SMILES: Cc1cccc2c1CN([C@H]1CC[C@@H](C(=O)Nc3ccc(Cl)c(F)c3)CC1)C2=O]

### (1s,4s)-N-(4-Chloro-3-fluorophenyl)-4-(4-methyl-1-oxoisoindolin-2-yl)cyclohexanecarboxamide

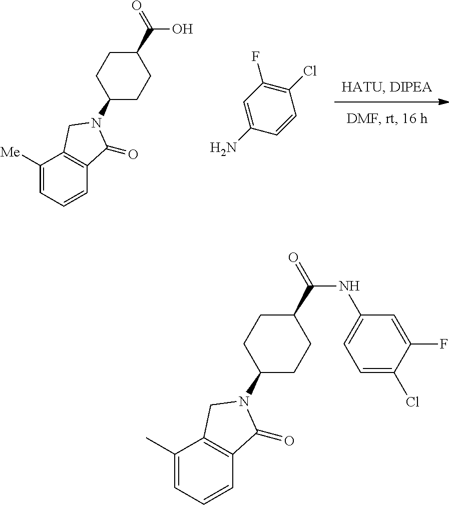 [SMILES: Cc1cccc2c1CN([C@H]1CC[C@@H](C(=O)O)CC1)C2=O ; Nc1ccc(Cl)c(F)c1 ; Cc1cccc2c1CN([C@H]1CC[C@@H](C(=O)Nc3ccc(Cl)c(F)c3)CC1)C2=O]

### Step 1

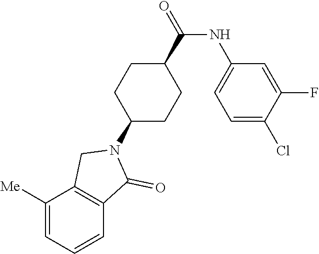 [SMILES: Cc1cccc2c1CN([C@H]1CC[C@@H](C(=O)Nc3ccc(Cl)c(F)c3)CC1)C2=O]

### (1s,4s)-N-(4-Chloro-3-fluorophenyl)-4-(4-methyl-1-oxoisoindolin-2-yl)cyclohexanecarboxamide

Following the synthetic procedure for Example 215, but starting with 4-chloro-3-fluoroaniline gave the title compound as a white solid. LC/MS ESI 400.8 [M+H]⁺. ¹H NMR (400 MHz, DMSO-d₆) δ 10.14 (s, 1H), 7.86 (dd, J=12.2, 2.2 Hz, 1H), 7.55-7.46 (m, 2H), 7.70-7.36 (m, 3H), 4.38 (s, 2H), 4.15-4.09 (m, 1H), 2.72-2.69 (m, 1H), 2.34 (s, 3H), 2.15-2.11 (m, 2H), 2.01-1.90 (m, 2H), 1.78-1.75 (m, 2H), 1.65-1.61 (m, 2H).

### Example 242

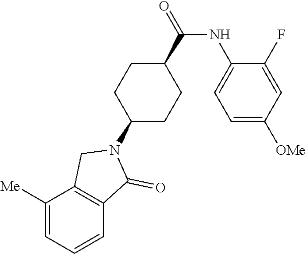 [SMILES: COc1ccc(NC(=O)[C@H]2CC[C@@H](N3Cc4c(C)cccc4C3=O)CC2)c(F)c1]

### (1s,4s)-N-(2-Fluoro-4-methoxyphenyl)-4-(4-methyl-1-oxoisoindolin-2-yl)cyclohexanecarboxamide

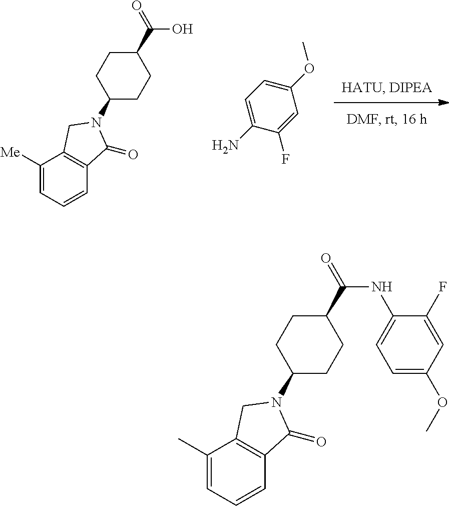

### Step 1

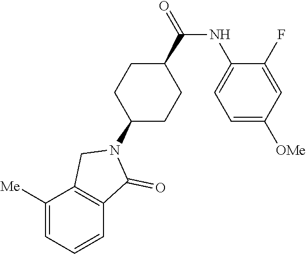 [SMILES: COc1ccc(NC(=O)[C@H]2CC[C@@H](N3Cc4c(C)cccc4C3=O)CC2)c(F)c1]

### (1s,4s)-N-(2-Fluoro-4-methoxyphenyl)-4-(4-methyl-1-oxoisoindolin-2-yl)cyclohexanecarboxamide

Following the synthetic procedure for Example 215, but starting with 2-fluoro-4-methoxyaniline gave the title compound as a white solid. LC/MS ESI 397.3 [M+H]⁺. ¹H NMR (400 MHz, DMSO-d₆) δ 9.40 (s, 1H), 7.53-7.47 (m, 2H), 7.40-7.35 (m, 2H), 6.88 (dd, J=12.4, 2.8 Hz, 1H), 6.78-6.73 (m, 1H), 4.35 (s, 2H), 4.14-4.09 (m, 1H), 3.76 (s, 3H), 2.75-2.73 (m, 1H), 2.33 (s, 3H), 2.15-2.12 (m, 2H), 2.02-1.93 (m, 2H), 1.72-1.67 (m, 2H), 1.63-1.59 (m, 2H).

### Example 243

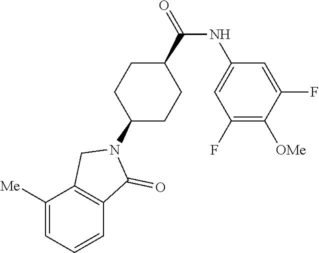 [SMILES: COc1c(F)cc(NC(=O)[C@H]2CC[C@@H](N3Cc4c(C)cccc4C3=O)CC2)cc1F]

### (1s,4s)-N-(3,5-Difluoro-4-methoxyphenyl)-4-(4-methyl-1-oxoisoindolin-2-yl)cyclohexanecarboxamide

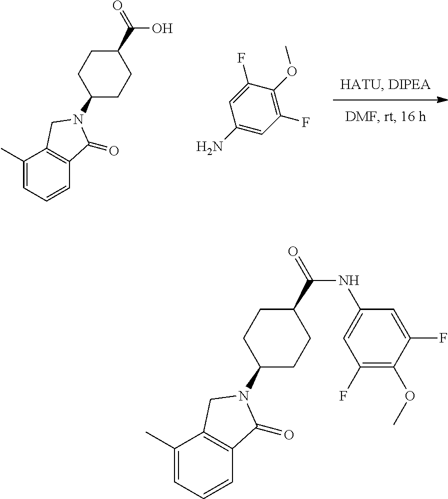 [SMILES: Cc1cccc2c1CN([C@H]1CC[C@@H](C(=O)O)CC1)C2=O ; COc1c(F)cc(N)cc1F ; COc1c(F)cc(NC(=O)[C@H]2CC[C@@H](N3Cc4c(C)cccc4C3=O)CC2)cc1F]

### Step 1

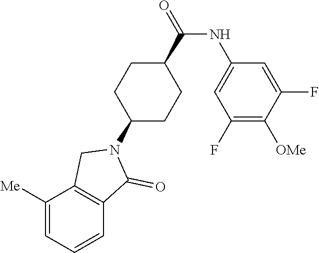 [SMILES: COc1c(F)cc(NC(=O)[C@H]2CC[C@@H](N3Cc4c(C)cccc4C3=O)CC2)cc1F]

### (1s,4s)-N-(3,5-Difluoro-4-methoxyphenyl)-4-(4-methyl-1-oxoisoindolin-2-yl)cyclohexanecarboxamide

Following the synthetic procedure for Example 215, but starting with 3,5-difluoro-4-methoxyaniline gave the title compound as a white solid. LC/MS ESI 415.2 [M+H]⁺. ¹H NMR (400 MHz, DMSO-d₆) δ 10.04 (s, 1H), 7.50-7.35 (m, 5H), 4.38 (s, 2H), 4.13-4.07 (m, 1H), 3.86 (s, 3H), 2.69-2.67 (m, 1H), 2.34 (s, 3H), 2.14-2.11 (m, 2H), 1.99-1.90 (m, 2H), 1.78-1.72 (m, 2H), 1.63-1.60 (m, 2H).

### Example 244

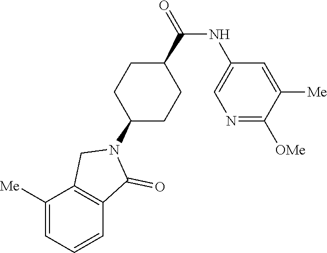 [SMILES: COc1ncc(NC(=O)[C@H]2CC[C@@H](N3Cc4c(C)cccc4C3=O)CC2)cc1C]

### (1s,4s)-N-(6-Methoxy-5-methylpyridin-3-yl)-4-(4-methyl-1-oxoisoindolin-2-yl)cyclohexanecarboxamide

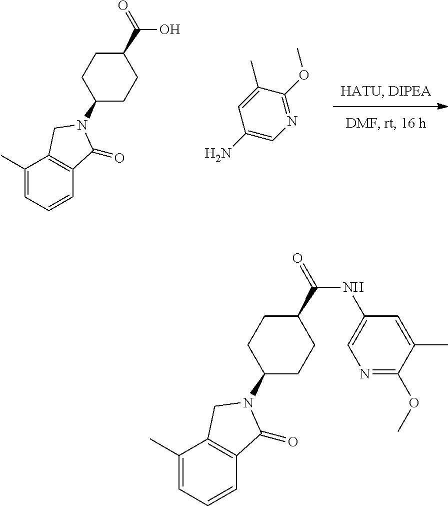

### Step 1

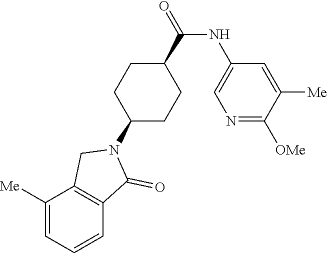 [SMILES: COc1ncc(NC(=O)[C@H]2CC[C@@H](N3Cc4c(C)cccc4C3=O)CC2)cc1C]

### (1s,4s)-N-(6-Methoxy-5-methylpyridin-3-yl)-4-(4-methyl-1-oxoisoindolin-2-yl)cyclohexanecarboxamide

Following the synthetic sequence for Example 215, but starting with 6-methoxy-5-methylpyridin-3-amine gave the title compound as a white solid. LC/MS ESI 394.2 [M+H]⁺. ¹H NMR (400 MHz, DMSO-d₆) δ 9.74 (s, 1H), 8.17 (d, J=2.3 Hz, 1H), 7.83 (d, J=1.8 Hz, 1H), 7.49-7.47 (m, 1H), 7.39-7.36 (m, 2H), 4.38 (s, 2H), 4.14-4.10 (m, 1H), 3.84 (s, 3H), 2.69-2.66 (m, 1H), 2.34 (s, 3H), 2.14-2.12 (m, 5H), 2.00-1.96 (m, 2H), 1.76-1.71 (m, 2H), 1.65-1.58 (m, 2H).

### Example 245

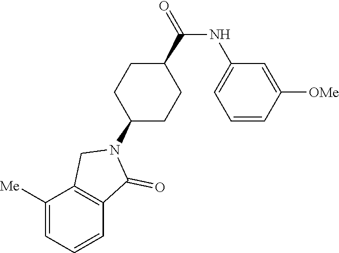 [SMILES: COc1cccc(NC(=O)[C@H]2CC[C@@H](N3Cc4c(C)cccc4C3=O)CC2)c1]

### (1s,4s)-N-(3-Methoxyphenyl)-4-(4-methyl-1-oxoisoindolin-2-yl)cyclohexanecarboxamide

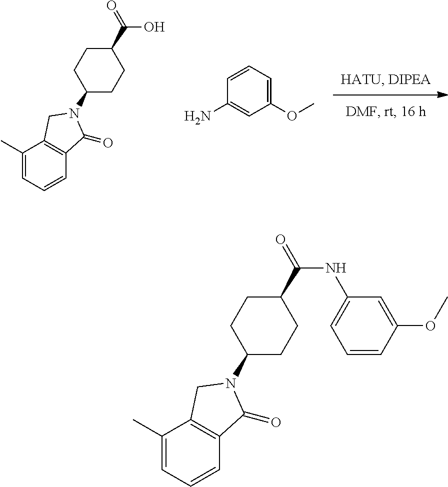

### Step 1

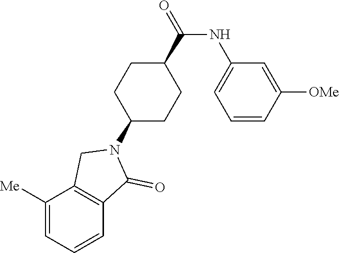 [SMILES: COc1cccc(NC(=O)[C@H]2CC[C@@H](N3Cc4c(C)cccc4C3=O)CC2)c1]

### (1s,4s)-N-(3-Methoxyphenyl)-4-(4-methyl-1-oxoisoindolin-2-yl)cyclohexanecarboxamide

Following the synthetic sequence for Example 215, but starting with 3-methoxyaniline gave the title compound as a white solid. LC/MS ESI 379.2 [M+H]⁺. ¹H NMR (400 MHz, DMSO-d₆) δ 9.77 (s, 1H), 7.49-7.47 (m, 1H), 7.40-7.34 (m, 3H), 7.24-7.16 (m, 2H), 6.64-6.58 (m, 1H), 4.38 (s, 2H), 4.16-4.07 (m, 1H), 3.73 (s, 3H), 2.69-2.67 (m, 1H), 2.34 (s, 3H), 2.14-2.11 (m, 2H), 2.01-1.96 (m, 2H), 1.76-1.73 (m, 2H), 1.63-1.59 (m, 2H).

### Example 246

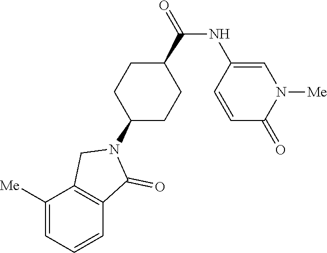 [SMILES: Cc1cccc2c1CN([C@H]1CC[C@@H](C(=O)Nc3ccc(=O)n(C)c3)CC1)C2=O]

### (1s,4s)-4-(4-Methyl-1-oxoisoindolin-2-yl)-N-(1-methyl-6-oxo-1,6-dihydropyridin-3-yl)cyclohexanecarboxamide

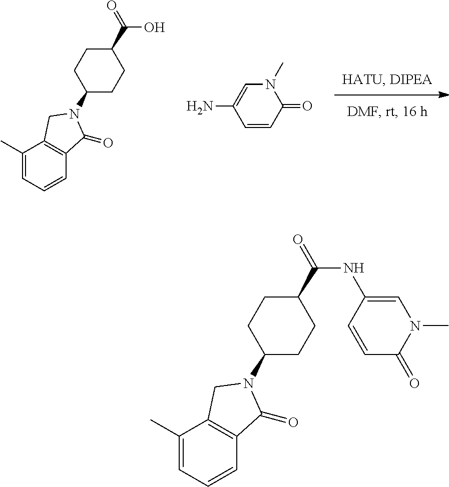

### Step 1

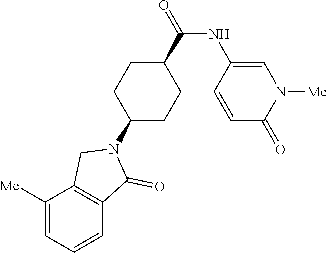 [SMILES: Cc1cccc2c1CN([C@H]1CC[C@@H](C(=O)Nc3ccc(=O)n(C)c3)CC1)C2=O]

### (1s,4s)-4-(4-Methyl-1-oxoisoindolin-2-yl)-N-(1-methyl-6-oxo-1,6-dihydropyridin-3-yl)cyclohexanecarboxamide

Following the synthetic procedure for Example 215, but starting with 5-amino-1-methylpyridin-2 (1H)-one gave the title compound as a white solid. LC/MS ESI 380.3 [M+H]⁺. ¹H NMR (400 MHz, DMSO-d₆) δ 9.55 (s, 1H), 8.16 (d, J=2.8 Hz, 1H), 7.51-7.46 (m, 1H), 7.42 (dd, J=9.6, 2.9 Hz, 1H), 7.38 (d, J=5.1 Hz, 2H), 6.39 (d, J=9.6 Hz, 1H), 4.36 (s, 2H), 4.13-4.08 (m, 1H), 3.43 (s, 3H), 2.65-2.63 (m, 1H), 2.34 (s, 3H), 2.13-2.09 (m, 2H), 2.01-1.92 (m, 2H), 1.75-1.72 (m, 2H), 1.62-1.58 (m, 2H).

### Example 247

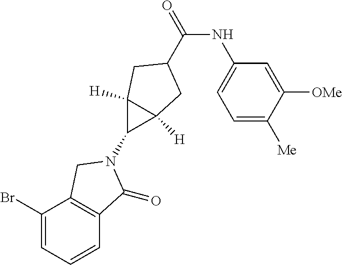 [SMILES: COc1cc(NC(=O)C2C[C@@H]3[C@H](C2)[C@H]3N2Cc3c(Br)cccc3C2=O)ccc1C]

### (1R,5S,6r)-6-(4-Bromo-1-oxoisoindolin-2-yl)-N-(3-methoxy-4-methylphenyl)bicyclo[3.1.0]hexane-3-carboxamide

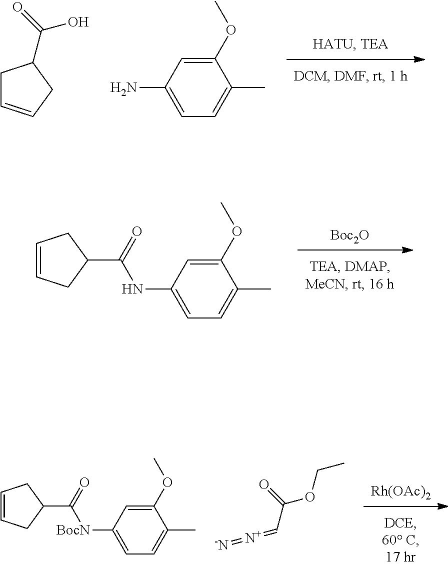

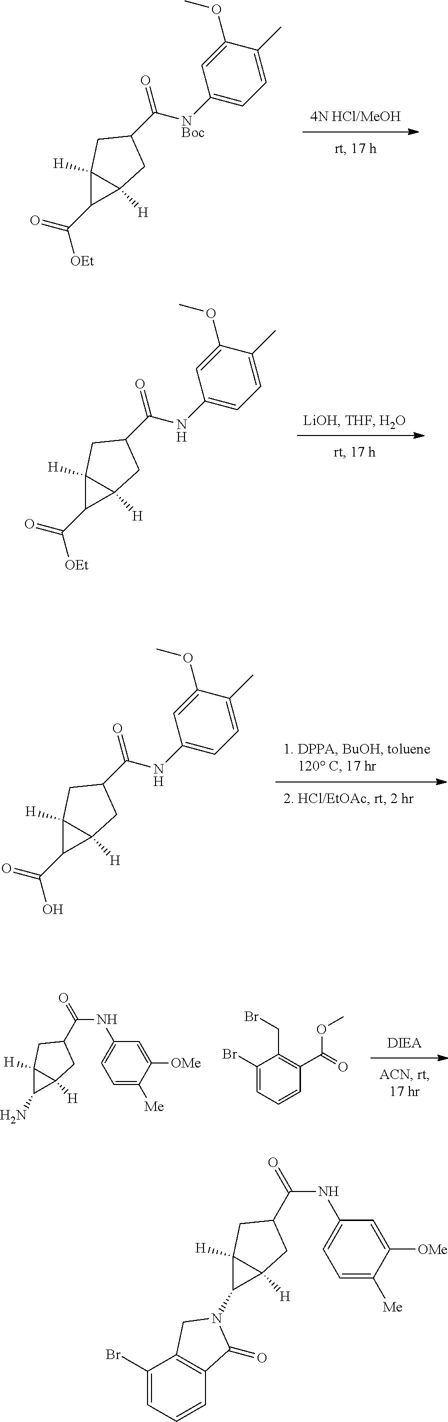 [SMILES: CCOC(=O)C1[C@H]2CC(C(=O)N(C(=O)OC(C)(C)C)c3ccc(C)c(OC)c3)C[C@@H]12 ; CCOC(=O)C1[C@H]2CC(C(=O)Nc3ccc(C)c(OC)c3)C[C@@H]12 ; COc1cc(NC(=O)C2C[C@@H]3C(C(=O)O)[C@@H]3C2)ccc1C ; COc1cc(NC(=O)C2C[C@@H]3[C@@H](N)[C@@H]3C2)ccc1C ; COC(=O)c1cccc(Br)c1CBr ; COc1cc(NC(=O)C2C[C@@H]3[C@H](C2)[C@H]3N2Cc3c(Br)cccc3C2=O)ccc1C]

### Step 1

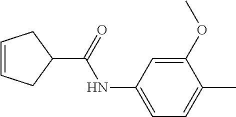 [SMILES: COc1cc(NC(=O)C2CC=CC2)ccc1C]

### N-(3-Methoxy-4-methylphenyl)cyclopent-3-enecarboxamide

A solution of 3-methoxy-4-methyl-aniline (5.00 g, 36.5 mmol), [dimethylamino (triazolo[4,5-b]pyridin-3-yloxy)methylene]-dimethyl-ammonium; hexafluorophosphate (15.24 g, 40.09 mmol) and N,N-diethylethanamine (11.1 g, 109 mmol, 15.2 mL) in DCM (20 mL) and DMF (20 mL) was stirred for 10 minutes. Cyclopent-3-ene-1-carboxylic acid (4.29 g, 38.3 mmol) was added, the mixture was stirred for 1 hour at 25° C. To the solution was added EtOAc (80 mL) and washed with H₂O (3×50 mL). The combined organic extracts were dried over Na₂SO₄ and evaporated in vacuo to get N-(3-methoxy-4-methyl-phenyl)cyclopent-3-ene-1-carboxamide (7.9 g, 34 mmol, 94% yield) as a brown solid. LC/MS ESI 232.2 [M+H]⁺.

### Step 2

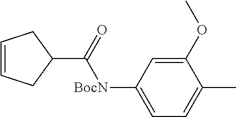 [SMILES: COc1cc(N(C(=O)OC(C)(C)C)C(=O)C2CC=CC2)ccc1C]

### tert-Butyl cyclopent-3-enecarbonyl(3-methoxy-4-methylphenyl)carbamate

A solution of N-(3-methoxy-4-methyl-phenyl)cyclopent-3-ene-1-carboxamide (6.00 g, 25.9 mmol) and tert-butoxycarbonyl tert-butyl carbonate (22.65 g, 103.8 mmol, 23.81 mL) and TEA (7.88 g, 77.8 mmol, 10.9 mL) and DMAP (317 mg, 2.59 mmol) in CH₃CN (20 mL) was stirred for 16 hours at 25° C. To the reaction mixture added EtOAc (50 mL) and washed with H₂O (3×50 mL). The combined organic extracts were dried over Na₂SO₄ and evaporated in vacuo. The residue was purified by flash chromatography on silica gel (0-10% EtOAc in PE) to get tert-butyl N-(cyclopent-3-ene-1-carbonyl)-N-(3-methoxy-4-methyl-phenyl)carbamate (8.00 g, 24.1 mmol, 93.1% yield) as a white solid. ¹H NMR (400 MHz, CDCl₃) δ 7.26 (d, J=1.8 Hz, 1H), 7.11 (d, J=4.0 Hz, 1H), 7.62-7.58 (m, 1H), 6.54 (s, 1H), 5.64 (s, 2H), 4.02-3.98 (m, 1H), 3.80 (s, 3H), 2.80-2.65 (m, 4H), 2.21 (s, 3H), 1.41 (s, 9H).

### Step 3

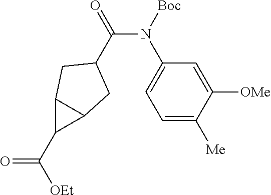 [SMILES: CCOC(=O)C1C2CC(C(=O)N(C(=O)OC(C)(C)C)c3ccc(C)c(OC)c3)CC21]

### Ethyl 3-(tert-butoxycarbonyl(3-methoxy-4-methylphenyl)carbamoyl)bicyclo[3.1.0]hexane-6-carboxylate

To a solution of tert-butyl N-(cyclopent-3-ene-1-carbonyl)-N-(3-methoxy-4-methyl-phenyl)carbamate (6.00 g, 18.1 mmol) in 1,2-dichloroethane (100 mL) was added rhodium (II) acetate dimer (160 mg, 360 μmol) under nitrogen. The mixture was heated to 60° C., then added ethyl diazoacetate (10.3 g, 90.5 mmol) dropwise to the reaction mixture. The resulting mixture was stirred at 85° C. for another 17 hours. The mixture was filtered through a celite pad and concentrated in vacuo. The residue was purified by silica gel chromatography (PE:EtOAc=30:1 to 10:1) to give ethyl 3-[tert-butoxycarbonyl-(3-methoxy-4-methyl-phenyl)carbamoyl]-bicyclo[3.1.0]hexane-6-carboxylate (2.5 g, 6.0 mmol, 33% yield) as yellow oil—a mixture of stereoisomers. LC/MS ESI 318.2 [M−100+H]⁺.

### Step 4

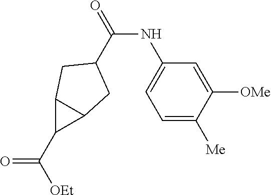 [SMILES: CCOC(=O)C1C2CC(C(=O)Nc3ccc(C)c(OC)c3)CC21]

### Ethyl 3-(3-methoxy-4-methylphenylcarbamoyl) bicyclo[3.1.0]hexane-6-carboxylate

A solution of ethyl 3-[tert-butoxycarbonyl-(3-methoxy-4-methyl-phenyl)carbamoyl]bicyclo[3.1.0]hexane-6-carboxylate (1.00 g, 2.40 mmol) in HCl/EtOAc (4M, 10 mL) was stirred at 25° C. for 17 hours. The mixture was concentrated in vacuum to give ethyl 3-(3-methoxy-4-methylphenylcarbamoyl) bicyclo[3.1.0]hexane-6-carboxylate (crude) as a white solid. LCMS ESI m/z 317.9 [M+H]⁺

### Step 5

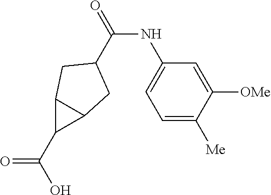 [SMILES: COc1cc(NC(=O)C2CC3C(C2)C3C(=O)O)ccc1C]

### 3-(3-Methoxy-4-methylphenylcarbamoyl) bicyclo[3.1.0]hexane-6-carboxylic acid

To a solution of ethyl 3-[(3-methoxy-4-methyl-phenyl)carbamoyl]bicyclo[3.1.0]hexane-6-carboxylate (700 mg, 2.21 mmol) in THF/H₂O (1/1, 5 mL) was added LiOH (254 mg, 11.0 mmol). The mixture was stirred at 25° C. for 17 hours. To the mixture was added 1 M HCl to adjust the solution to PH~6. The mixture was extracted with EtOAc (20 mL*3). The organic phases were concentrated to give 3-(3-methoxy-4-methylphenylcarbamoyl) bicyclo[3.1.0]hexane-6-carboxylic acid (500 mg, 1.7 mmol, 78% yield) as a white solid. LCMS ESI m/z 289.9 [M+H]⁺

### Step 6

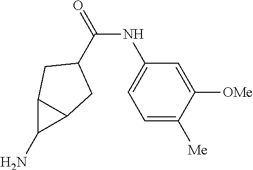 [SMILES: COc1cc(NC(=O)C2CC3C(N)C3C2)ccc1C]

### 6-Amino-N-(3-methoxy-4-methylphenyl)bicyclo[3.1.0]hexane-3-carboxamide

To a solution of 3-[(3-methoxy-4-methyl-phenyl)carbamoyl]bicycle [3.1.0]hexane-6-carboxylic acid (300 mg, 1.04 mmol), N-ethyl-N-isopropyl-propan-2-amine (161 mg, 1.24 mmol, 217 L) and [azido (phenoxy)phosphoryl]oxybenzene (342 mg, 1.24 mmol, 269 μL) in toluene (15 mL) and BuOH (10 mL) was stirred at 120° C. for 17 hours. The reaction mixture was poured into water and extracted with EtOAc. The organic phase was concentrated in vacuo. To the residue was added HCl/EtOAc (4M, 5 mL) and the resulting mixture was stirred at room temperature for 2 hours. To the mixture was added sat. NaHCO₃ solution to adjust pH~7, and extracted with EtOAc. The organic layers were dried and concentrated. The residue was purified by silica gel chromatography to give 6-amino-N-(3-methoxy-4-methylphenyl)bicyclo[3.1.0]hexane-3-carboxamide (140 mg, 538 μmol) as a yellow solid. LCMS ESI m/z 260.9 [M+H]⁺

### Step 7

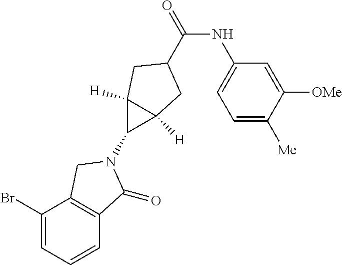 [SMILES: COc1cc(NC(=O)C2C[C@@H]3[C@H](C2)[C@H]3N2Cc3c(Br)cccc3C2=O)ccc1C]

### (1R,5S,6r)-6-(4-Bromo-1-oxoisoindolin-2-yl)-N-(3-methoxy-4-methylphenyl)bicyclo[3.1.0]hexane-3-carboxamide

To a solution of 6-amino-N-(3-methoxy-4-methyl-phenyl)bicyclo[3.1.0]hexane-3-carboxamide (115 mg, 441.75 mmol) and methyl 3-bromo-2-(bromomethyl)benzoate (150 mg, 486 mmol) in MeCN (10 mL) was added DIPEA (114 mg, 883 mmol). The mixture was stirred at 25° C. for 2 hours. The mixture was poured into water (10 mL) and extracted with EtOAc (15 mL). The organic layer was dried and concentrated. The residue was purified by prep-HPLC to give racemic (1R,5S,6r)-6-(4-bromo-1-oxoisoindolin-2-yl)-N-(3-methoxy-4-methylphenyl)bicyclo[3.1.0]hexane-3-carboxamide (10 mg, 22 mmol, 5.0% yield) as a white solid. The product was isolated as a single isomer and arbitrarily assigned (1R,5S,6r). LCMS ESI m/z 454.8 [M+H]⁺. ¹HNMR (400 MHz, DMSO-d₆) δ 9.92 (s, 1H), 7.87 (dd, J=7.9, 0.6 Hz, 1H), 7.75 (d, J=8.0 Hz, 1H), 7.53 (t, J=7.7 Hz, 1H), 7.38 (d, J=1.3 Hz, 1H), 7.16-7.00 (m, 2H), 4.42 (s, 2H), 3.81 (s, 3H), 2.76-2.74 (m, 1H), 2.63-2.60 (m, 1H), 2.26-2.21 (m, 2H), 2.18-2.04 (m, 5H), 1.99-1.97 (m, 2H).

### Example 248

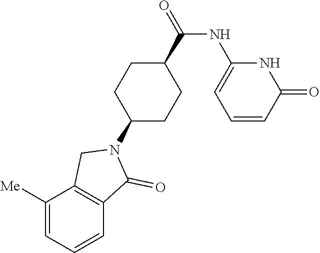 [SMILES: Cc1cccc2c1CN([C@H]1CC[C@@H](C(=O)Nc3cccc(=O)[nH]3)CC1)C2=O]

### (1s,4s)-4-(4-Methyl-1-oxoisoindolin-2-yl)-N-(6-oxo-1,6-dihydropyridin-2-yl)cyclohexanecarboxamide

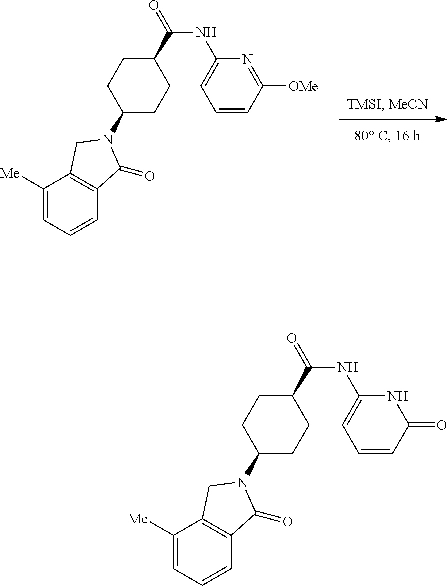 [SMILES: COc1cccc(NC(=O)[C@H]2CC[C@@H](N3Cc4c(C)cccc4C3=O)CC2)n1 ; Cc1cccc2c1CN([C@H]1CC[C@@H](C(=O)Nc3cccc(=O)[nH]3)CC1)C2=O]

### Step 1

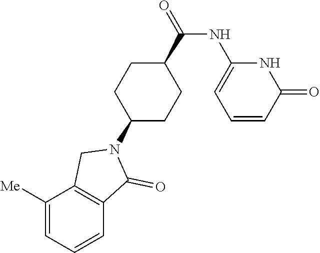 [SMILES: Cc1cccc2c1CN([C@H]1CC[C@@H](C(=O)Nc3cccc(=O)[nH]3)CC1)C2=O]

### (1s,4s)-4-(4-Methyl-1-oxoisoindolin-2-yl)-N-(6-oxo-1,6-dihydropyridin-2-yl)cyclohexanecarboxamide

A mixture of N-(6-methoxy-2-pyridyl)-4-(4-methyl-1-oxo-isoindolin-2-yl)cyclohexanecarboxamide (0.080 g, 210 μmol) and iodo (trimethyl) silane (422 mg, 2.11 mmol, 293 μL) in CH₃CN (10 mL) was stirred for 16 hours at 80° C. The solvent was evaporated in vacuo to dryness, then added H₂O (50 mL), extracted with EtOAc (3×50 mL). The combined organic extracts were dried over Na₂SO₄ and evaporated in vacuo. The residue was purified by flash chromatography on silica gel (0-10% MeOH in DCM) and concentrated. The residue was further purified by Prep-HPLC to get (1s,4s)-4-(4-methyl-1-oxoisoindolin-2-yl)-N-(6-oxo-1,6-dihydropyridin-2-yl)cyclohexanecarboxamide (0.0105 g, 28.7 μmol, 13.6% yield) as a white solid. LCMS ESI m/z: 365.9 [M+H]⁺. ¹H NMR (400 MHz, CDCl₃) δ 10.08 (s, 1H), 7.65 (d, J=7.2 Hz, 1H), 7.55-7.45 (m, 1H), 7.40-7.30 (m, 2H), 6.69 (d, J=7.6 Hz, 1H), 6.23 (d, J=8.8 Hz, 1H), 4.32-4.28 (m, 3H), 2.78-2.76 (m, 1H), 2.36 (s, 3H), 2.32-2.20 (m, 2H), 2.10-1.95 (m, 2H), 1.90-1.70 (m, 4H).

### Example 249

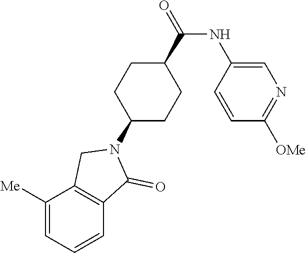 [SMILES: COc1ccc(NC(=O)[C@H]2CC[C@@H](N3Cc4c(C)cccc4C3=O)CC2)cn1]

### (1s,4s)-N-(6-Methoxypyridin-3-yl)-4-(4-methyl-1-oxoisoindolin-2-yl)cyclohexanecarboxamide

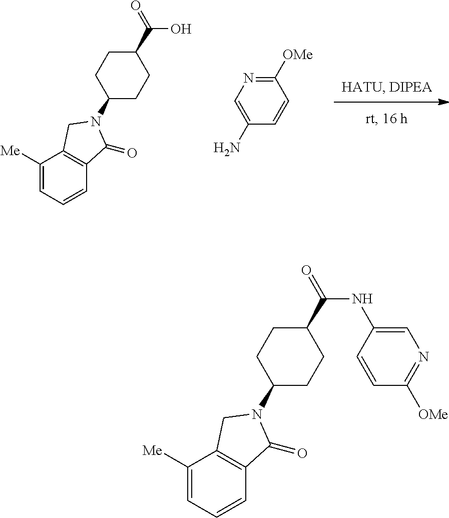 [SMILES: Cc1cccc2c1CN([C@H]1CC[C@@H](C(=O)O)CC1)C2=O ; COc1ccc(N)cn1 ; COc1ccc(NC(=O)[C@H]2CC[C@@H](N3Cc4c(C)cccc4C3=O)CC2)cn1]

### Step 1

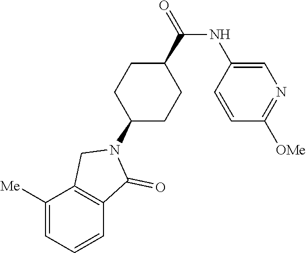 [SMILES: COc1ccc(NC(=O)[C@H]2CC[C@@H](N3Cc4c(C)cccc4C3=O)CC2)cn1]

### (1s,4s)-N-(6-Methoxypyridin-3-yl)-4-(4-methyl-1-oxoisoindolin-2-yl)cyclohexanecarboxamide

Following the synthetic procedure for Example 215, but starting with 6-methoxypyridin-3-amine gave the title compound as a white solid. LC/MS ESI 379.9 [M+H]⁺. ¹H NMR (400 MHz, DMSO-d₆) δ 9.85 (s, 1H), 8.37 (d, J=2.4 Hz, 1H), 8.05-7.89 (m, 1H), 7.55-7.44 (m, 1H), 7.44-7.29 (m, 2H), 6.80 (d, J=8.8 Hz, 1H), 4.38 (s, 2H), 4.20-4.05 (m, 1H), 3.82 (s, 3H), 2.71-2.69 (m, 1H), 2.34 (s, 3H), 2.25-2.10 (m, 2H), 2.05-1.90 (m 2H), 1.81-1.54 (m, 4H).

### Example 250

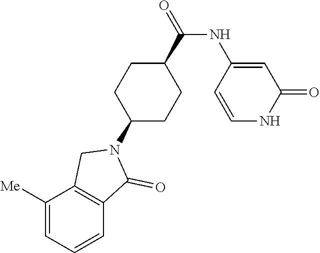 [SMILES: Cc1cccc2c1CN([C@H]1CC[C@@H](C(=O)Nc3cc[nH]c(=O)c3)CC1)C2=O]

### (1s,4s)-4-(4-methyl-1-oxoisoindolin-2-yl)-N-(2-oxo-1,2-dihydropyridin-4-yl)cyclohexanecarboxamide

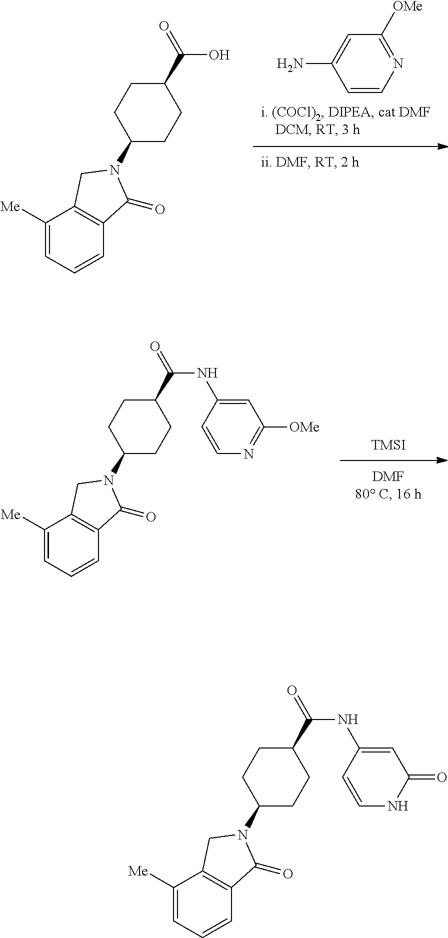 [SMILES: Cc1cccc2c1CN([C@H]1CC[C@@H](C(=O)O)CC1)C2=O ; "TMSI" ; COc1cc(N)ccn1 ; COc1cc(NC(=O)[C@H]2CC[C@@H](N3Cc4c(C)cccc4C3=O)CC2)ccn1 ; Cc1cccc2c1CN([C@H]1CC[C@@H](C(=O)Nc3cc[nH]c(=O)c3)CC1)C2=O]

### Step 1

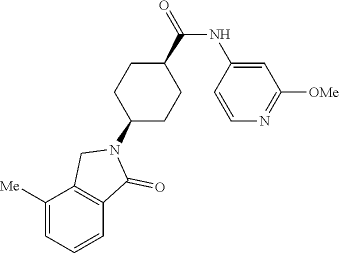 [SMILES: COc1cc(NC(=O)[C@H]2CC[C@@H](N3Cc4c(C)cccc4C3=O)CC2)ccn1]

### (1s,4s)-N-(2-Methoxypyridin-4-yl)-4-(4-methyl-1-oxoisoindolin-2-yl)cyclohexanecarboxamide

To a solution of 4-(4-methyl-1-oxo-isoindolin-2-yl)cyclohexanecarboxylic acid (350 mg, 1.28 mmol) in DCM (5.0 mL) was added oxalyl chloride (325 mg, 2.56 mmol, 223 mL) and one drop of DMF at 0° C. under nitrogen. The mixture was stirred at 25° C. for 3 hours. The mixture was concentrated to give 4-(4-methyl-1-oxo-isoindolin-2-yl)cyclohexanecarbonyl chloride (350 mg, crude) as a yellow oil. To a solution of 4-(4-methyl-1-oxo-isoindolin-2-yl)cyclohexanecarbonyl chloride (350 mg, 1.2 mmol) and 2-methoxypyridin-4-amine (194 mg, 1.56 mmol) in DMF (5 mL) was added DIPEA (775 mg, 6.00 mmol, 1.04 mL). The mixture was stirred at 25° C. for 2 hours. The mixture was poured into water (10 mL) and extracted with EtOAc (10 mL*2). The organic extracts were washed with brine, dried by Na₂SO₄, filtered and concentrated to give a crude oil. The crude product was purified by silica gel chromatography to give (1s,4s)-N-(2-methoxypyridin-4-yl)-4-(4-methyl-1-oxoisoindolin-2-yl)cyclohexanecarboxamide (90 mg, 210 mmol, 18% yield) as a yellow solid. LCMS ESI m/z: 379.9 [M+H]⁺.

### Step 2

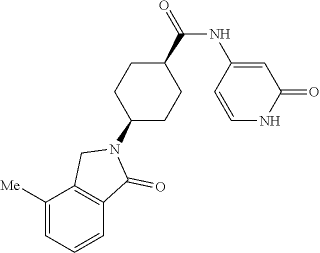 [SMILES: Cc1cccc2c1CN([C@H]1CC[C@@H](C(=O)Nc3cc[nH]c(=O)c3)CC1)C2=O]

### (1s,4s)-4-(4-Methyl-1-oxoisoindolin-2-yl)-N-(2-oxo-1,2-dihydropyridin-4-yl)cyclohexanecarboxamide

A solution of N-(2-methoxy-4-pyridyl)-4-(4-methyl-1-oxo-isoindolin-2-yl)cyclohexanecarboxamide (80 mg, 210 mmol) and TMSI (420 mg, 2.1 mmol) in DMF (3 mL) was stirred at 80° C. for 16 hours. The mixture was poured into water (10 mL) and extracted with EtOAc (10 mL*3). The organic extracts were washed with brine, dried by Na₂SO₄, filtered and concentrated to give a crude oil. The crude product was purified by silica gel chromatography to give a yellow solid (80 mg). The yellow solid was purified by prep-HPLC to give (1s,4s)-4-(4-methyl-1-oxoisoindolin-2-yl)-N-(2-oxo-1,2-dihydropyridin-4-yl)cyclohexanecarboxamide (19 mg, 52 mmol, 25% yield) as a white solid. LCMS ESI m/z: 365.9 [M+H]⁺. ¹H NMR (400 MHz, DMSO-d₆) δ 11.12 (br, 1H), 9.91 (s, 1H), 7.51-7.46 (m, 1H), 7.41-7.36 (m, 2H), 7.27 (d, J=7.2 Hz, 1H), 6.78 (d, J=2.0 Hz, 1H), 6.37 (dd, J=7.2, 2.1 Hz, 1H), 4.39 (s, 2H), 4.15-4.07 (m, 1H), 2.71-2.69 (m, 1H), 2.35 (s, 3H), 2.12-2.08 (m, 2H), 1.99-1.91 (m, 2H), 1.77-1.71 (m, 2H), 1.66-1.58 (m, 2H).

### Example 251

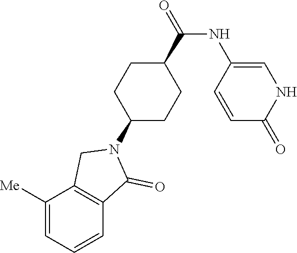 [SMILES: Cc1cccc2c1CN([C@H]1CC[C@@H](C(=O)Nc3ccc(=O)[nH]c3)CC1)C2=O]

### (1s,4s)-4-(4-Methyl-1-oxoisoindolin-2-yl)-N-(6-oxo-1,6-dihydropyridin-3-yl)cyclohexanecarboxamide

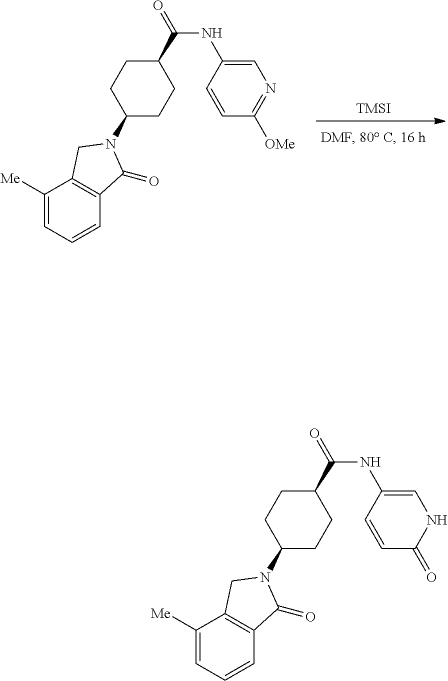 [SMILES: "TMSI" ; COc1ccc(NC(=O)[C@H]2CC[C@@H](N3Cc4c(C)cccc4C3=O)CC2)cn1 ; Cc1cccc2c1CN([C@H]1CC[C@@H](C(=O)Nc3ccc(=O)[nH]c3)CC1)C2=O]

### Step 1

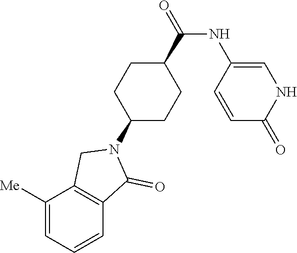 [SMILES: Cc1cccc2c1CN([C@H]1CC[C@@H](C(=O)Nc3ccc(=O)[nH]c3)CC1)C2=O]

### (1s,4s)-4-(4-Methyl-1-oxoisoindolin-2-yl)-N-(6-oxo-1,6-dihydropyridin-3-yl)cyclohexanecarboxamide

Following the synthetic procedure for Example 250, but starting with (1s,4s)-N-(6-methoxypyridin-3-yl)-4-(4-methyl-1-oxoisoindolin-2-yl)cyclohexanecarboxamide gave the title compound as a white solid. LC/MS ESI 366.0 [M+H]⁺. ¹H NMR (400 MHz, DMSO-d₆) δ 11.33 (br, 1H), 9.53 (s, 1H), 7.86 (d, J=2.4 Hz, 1H), 7.55-7.45 (m, 2H), 7.42-7.30 (m, 2H), 6.35 (d, J=9.6 Hz, 1H), 4.37 (s, 2H), 4.15-4.05 (m, 1H), 2.64-2.62 (m, 1H), 2.34 (s, 3H), 2.20-2.05 (m, 2H), 2.03-1.89 (m, 2H), 1.78-1.54 (m, 4H).

### Example 252

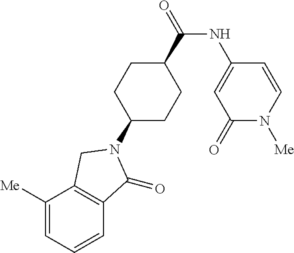 [SMILES: Cc1cccc2c1CN([C@H]1CC[C@@H](C(=O)Nc3ccn(C)c(=O)c3)CC1)C2=O]

### (1s,4s)-4-(4-Methyl-1-oxoisoindolin-2-yl)-N-(1-methyl-2-oxo-1,2-dihydropyridin-4-yl)cyclohexanecarboxamide

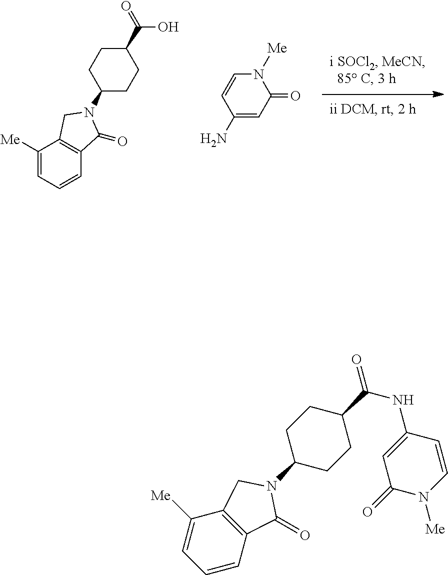 [SMILES: Cc1cccc2c1CN([C@H]1CC[C@@H](C(=O)O)CC1)C2=O ; Cn1ccc(N)cc1=O ; Cc1cccc2c1CN([C@H]1CC[C@@H](C(=O)Nc3ccn(C)c(=O)c3)CC1)C2=O]

### Step 1

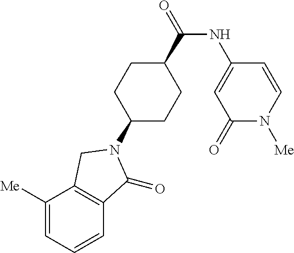 [SMILES: Cc1cccc2c1CN([C@H]1CC[C@@H](C(=O)Nc3ccn(C)c(=O)c3)CC1)C2=O]

### (1s,4s)-4-(4-Methyl-1-oxoisoindolin-2-yl)-N-(1-methyl-2-oxo-1,2-dihydropyridin-4-yl)cyclohexanecarboxamide

To a solution of 4-(4-methyl-1-oxo-isoindolin-2-yl)cyclohexanecarboxylic acid (0.20 g, 730 mmol) in CH₃CN (5.1 mL) was added SOCl₂ (0.20 mL). The mixture was stirred at 85° C. for 3 hours. The reaction solution was concentrated to give 4-(4-methyl-1-oxo-isoindolin-2-yl)cyclohexanecarbonyl chloride (0.16 g, 550 mmol, 75% yield) as a yellow oil. To a solution of 4-(4-methyl-1-oxo-isoindolin-2-yl)cyclohexanecarbonyl chloride (0.16 g, 550 μmol) and 4-amino-1-methyl-pyridin-2-one (106 mg, 658 μmol, hydrochloride salt) in DCM (850 μL) was added DIPEA (142 mg, 1.10 mmol, 191 μL). The mixture was stirred for 2 hours at 25° C. To the reaction mixture was added EtOAc (50 mL) and washed with H₂O (3×50 mL). The combined organic extracts were dried over Na₂SO₄ and evaporated in vacuo. The residue was purified by flash chromatography on silica gel (0-10% EtOAc in PE; MeOH in DCM) to get product that was further purified by Prep-HPLC to get (1s,4s)-4-(4-methyl-1-oxoisoindolin-2-yl)-N-(1-methyl-2-oxo-1,2-dihydropyridin-4-yl)cyclohexanecarboxamide (0.012 g, 32 μmol, 5.8% yield) as a white solid. LCMS ESI m/z: 379.9 [M+H]⁺. ¹H NMR (400 MHz, MeOD-d₄) δ 7.62-7.50 (m, 2H), 7.38 (d, J=4.4 Hz, 2H), 7.02 (d, J=2.4 Hz, 1H), 6.70-6.60 (m, 1H), 4.46 (s, 2H), 4.30-4.20 (m, 1H), 3.51 (s, 3H), 2.75-2.73 (m, 1H), 2.38 (s, 3H), 2.25-2.05 (m, 4H), 1.89-1.70 (m, 4H).

### Example 253

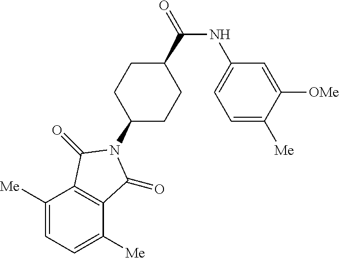 [SMILES: COc1cc(NC(=O)[C@H]2CC[C@@H](N3C(=O)c4c(C)ccc(C)c4C3=O)CC2)ccc1C]

### (1s,4s)-4-(4,7-Dimethyl-1,3-dioxoisoindolin-2-yl)-N-(3-methoxy-4-methylphenyl)cyclohexanecarboxamide

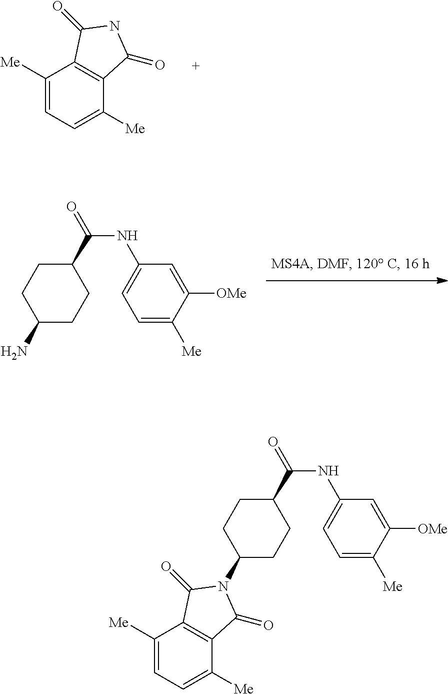 [SMILES: Cc1ccc(C)c2c1C(=O)NC2=O ; COc1cc(NC(=O)[C@H]2CC[C@@H](N)CC2)ccc1C ; COc1cc(NC(=O)[C@H]2CC[C@@H](N3C(=O)c4c(C)ccc(C)c4C3=O)CC2)ccc1C]

### Step 1

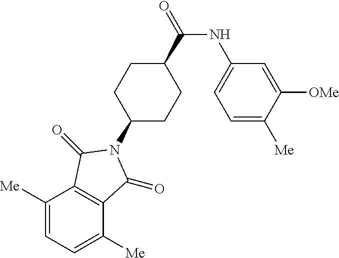 [SMILES: COc1cc(NC(=O)[C@H]2CC[C@@H](N3C(=O)c4c(C)ccc(C)c4C3=O)CC2)ccc1C]

### (1s,4s)-4-(4,7-Dimethyl-1,3-dioxoisoindolin-2-yl)-N-(3-methoxy-4-methylphenyl)cyclohexanecarboxamide

A mixture of 4,7-dimethylisobenzofuran-1,3-dione (100 mg, 568 mmol), 4-amino-N-(3-methoxy-4-methyl-phenyl)cyclohexanecarboxamide (149 mg, 568 mmol) and 4 angstrom molecular sieves (MS4A, 50 mg) was stirred at 120° C. under argon gas atmosphere for 16 hours. The reaction mixture was filtered and concentrated. Then the crude was purified by Prep-TLC (PE:EtOAc=3:1) to give (1s,4s)-4-(4,7-dimethyl-1,3-dioxoisoindolin-2-yl)-N-(3-methoxy-4-methylphenyl)cyclohexanecarboxamide (56 mg, 130 mmol, 23% yield) as a white solid. LC/MS ESI 421.2 [M+H]⁺. ¹H NMR (400 MHz, DMSO-d₆) 09.60 (s, 1H), 7.44 (s, 2H), 7.35 (d, J=1.6 Hz, 1H), 7.13 (dd, J=8.0, 1.7 Hz, 1H), 7.03 (d, J=8.1 Hz, 1H), 4.02-3.96 (m, 1H), 3.78 (s, 3H), 2.68-2.66 (m, 1H), 2.55 (s, 6H), 2.46-2.43 (m, 2H), 2.22-2.18 (m, 2H), 2.09 (s, 3H), 1.71-1.58 (m, 2H), 1.54-1.52 (m, 2H).

### Example 254

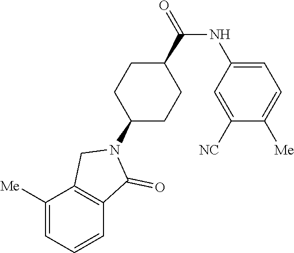 [SMILES: Cc1ccc(NC(=O)[C@H]2CC[C@@H](N3Cc4c(C)cccc4C3=O)CC2)cc1C#N]

### (1s,4s)-N-(3-Cyano-4-methylphenyl)-4-(4-methyl-1-oxoisoindolin-2-yl)cyclohexanecarboxamide

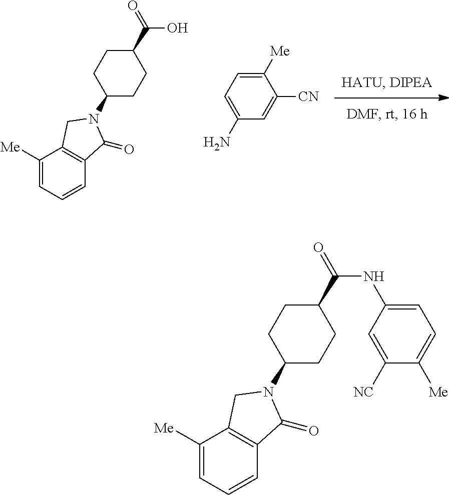 [SMILES: Cc1cccc2c1CN([C@H]1CC[C@@H](C(=O)O)CC1)C2=O ; Cc1ccc(N)cc1C#N ; Cc1ccc(NC(=O)[C@H]2CC[C@@H](N3Cc4c(C)cccc4C3=O)CC2)cc1C#N]

### Step 1

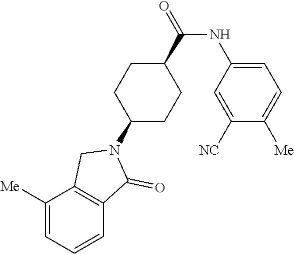 [SMILES: Cc1ccc(NC(=O)[C@H]2CC[C@@H](N3Cc4c(C)cccc4C3=O)CC2)cc1C#N]

### (1s,4s)-N-(3-Cyano-4-methylphenyl)-4-(4-methyl-1-oxoisoindolin-2-yl)cyclohexanecarboxamide

Following the synthetic procedure for Example 215, but starting with 5-amino-1-methylpyridin-2(1H)-one gave the title compound as a white solid. LC/MS ESI 387.9 [M+H]⁺. ¹H NMR (400 MHz, DMSO-d₆) δ 10.05 (s, 1H), 8.11 (d, J=2.2 Hz, 1H), 7.72 (dd, J=8.4, 2.2 Hz, 1H), 7.52-7.45 (m, 1H), 7.43-7.34 (m, 3H), 4.38 (s, 2H), 4.15-4.09 (m, 1H), 2.71-2.70 (m, 1H), 2.43 (s, 3H), 2.34 (s, 3H), 2.18-2.08 (m, 2H), 2.00-1.95 (m, 2H), 1.77-1.74 (m, 2H), 1.66-1.58 (m, 2H).

### Example 255

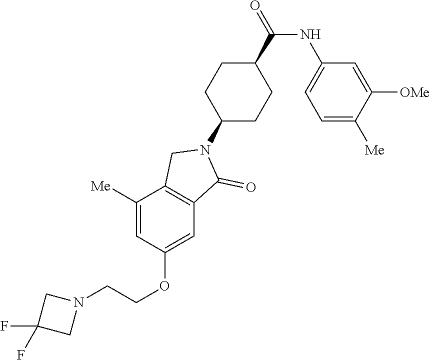 [SMILES: COc1cc(NC(=O)[C@H]2CC[C@@H](N3Cc4c(C)cc(OCCN5CC(F)(F)C5)cc4C3=O)CC2)ccc1C]

### (1s,4s)-N-(3-Cyano-4-methylphenyl)-4-(4-methyl-1-oxoisoindolin-2-yl)cyclohexanecarboxamide

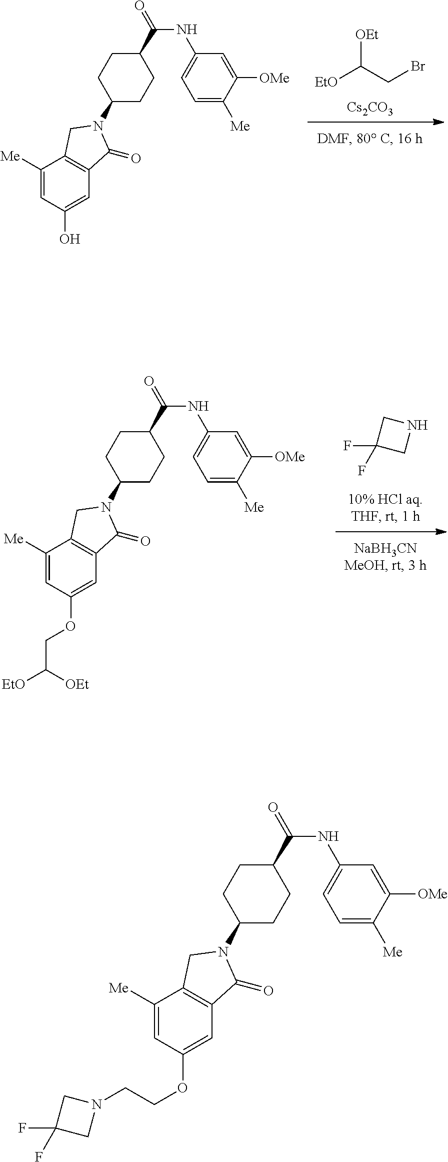

### Step 1

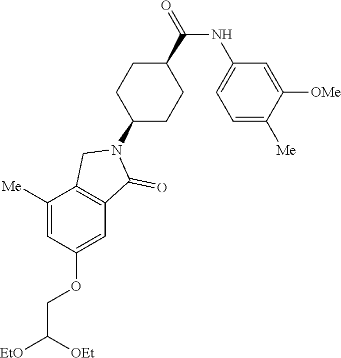 [SMILES: CCOC(COc1cc(C)c2c(c1)C(=O)N([C@H]1CC[C@@H](C(=O)Nc3ccc(C)c(OC)c3)CC1)C2)OCC]

### (1s,4s)-4-(6-(2,2-Diethoxyethoxy)-4-methyl-1-oxoisoindolin-2-yl)-N-(3-methoxy-4-methylphenyl)cyclohexanecarboxamide

A solution of 4-(6-hydroxy-4-methyl-1-oxo-isoindolin-2-yl)-N-(3-methoxy-4-methyl-phenyl)cyclohexanecarboxamide (400 mg, 980 mmol) and Cs₂CO₃ (960 mg, 2.9 mmol) in DMF (0.50 mL) was stirred at 80° C. for 16 hours. The mixture was poured into water and extracted with EtOAc. The organic extracts were washed with brine, dried by Na₂SO₄, filtered and concentrated to give a crude oil. The crude product was purified by silica gel chromatography to give (1s,4s)-4-(6-(2,2-diethoxyethoxy)-4-methyl-1-oxoisoindolin-2-yl)-N-(3-methoxy-4-methylphenyl)cyclohexanecarboxamide (400 mg, 530 mmol, 55% yield, 70% purity) as a yellow oil. LCMS ESI m/z: 524.2 [M+H]⁺.

### Step 2

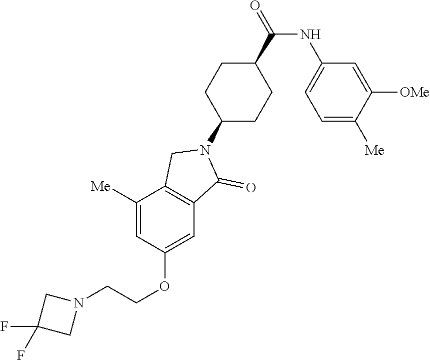 [SMILES: COc1cc(NC(=O)[C@H]2CC[C@@H](N3Cc4c(C)cc(OCCN5CC(F)(F)C5)cc4C3=O)CC2)ccc1C]

### (1s,4s)-4-(6-(2-(3,3-Difluoroazetidin-1-yl) ethoxy)-4-methyl-1-oxoisoindolin-2-yl)-N-(3-methoxy-4-methylphenyl)cyclohexanecarboxamide

A solution of 4-[6-(2,2-diethoxyethoxy)-4-methyl-1-oxo-isoindolin-2-yl]-N-(3-methoxy-4-methyl-phenyl)cyclohexanecarboxamide (80 mg, 150 mmol) and 10% HCl aq. (4.0 mL) in THF (4.0 mL) was stirred at 25° C. for 1 hour. The mixture was concentrated to give a white solid. The crude was dissolved in MeOH (5.0 mL), 3,3-difluoroazetidine (20 mg, 160 mmol, hydrochloride salt) and sodium cyanoborohydride (49 mg, 780 mmol) was added to the reaction mixture. The mixture was stirred at 25° C. for 3 hours. The mixture was mixed with saturated sodium carbonate aqueous solution (20 mL), extracted with EtOAc. The organic extracts were washed with brine, dried by Na₂SO₄, filtered and concentrated to give a crude oil. The crude product was purified by silica gel chromatography to give 4-[6-[2-(3,3-difluoroazetidin-1-yl) ethoxy]-4-methyl-1-oxo-isoindolin-2-yl]-N-(3-methoxy-4-methyl-phenyl)cyclohexanecarboxamide (32 mg, 59 mmol, 38% yield) as a white solid. LCMS ESI m/z: 527.8 [M+H]⁺. ¹H NMR (400 MHz, DMSO-d₆) δ 9.70 (s, 1H), 7.32 (d, J=1.7 Hz, 1H), 7.12 (dd, J=8.0, 1.9 Hz, 1H), 7.07-6.92 (m, 3H), 4.30 (s, 2H), 4.12-4.08 (m, 1H), 4.04 (t, J=5.3 Hz, 2H), 3.76 (s, 3H), 3.66 (t, J=12.5 Hz, 4H), 2.91 (t, J=5.2 Hz, 2H), 2.68-2.66 (m, 1H), 2.29 (s, 3H), 2.16-2.05 (m, 5H), 2.02-1.95 (m, 2H), 1.75-1.71 (m, 2H), 1.64-1.56 (m, 2H).

### Example 256

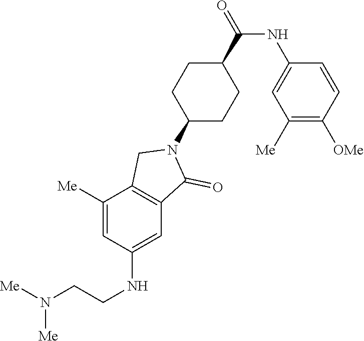 [SMILES: COc1ccc(NC(=O)[C@H]2CC[C@@H](N3Cc4c(C)cc(NCCN(C)C)cc4C3=O)CC2)cc1C]

### (1s,4s)-4-(6-(2-(Dimethylamino)ethylamino)-4-methyl-1-oxoisoindolin-2-yl)-N-(4-methoxy-3-methylphenyl)cyclohexanecarboxamide

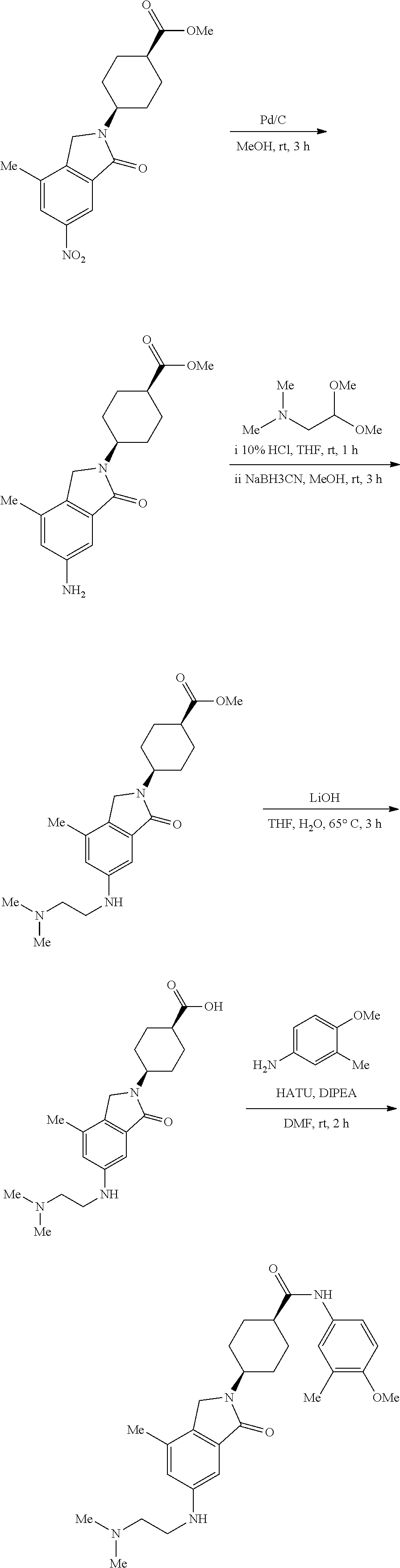

### Step 1

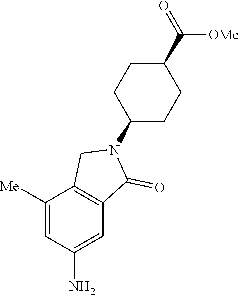 [SMILES: COC(=O)[C@H]1CC[C@@H](N2Cc3c(C)cc(N)cc3C2=O)CC1]

### (1s,4s)-Methyl 4-(6-amino-4-methyl-1-oxoisoindolin-2-yl)cyclohexanecarboxylate

A solution of methyl 4-(4-methyl-6-nitro-1-oxo-isoindolin-2-yl)cyclohexanecarboxylate (400 mg, 1.20 mmol) and Pd/C (440 mg, 10% Pd wt) in MeOH (10 mL) was stirred at 25° C. for 3 hours. The mixture was filtered and concentrated to give methyl 4-(6-amino-4-methyl-1-oxo-isoindolin-2-yl)cyclohexanecarboxylate (360 mg, crude) as a yellow solid. LCMS ESI m/z: 303.2 [M+H]⁺.

### Step 2

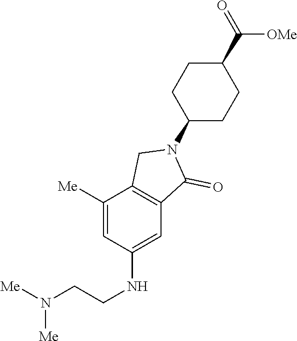 [SMILES: COC(=O)[C@H]1CC[C@@H](N2Cc3c(C)cc(NCCN(C)C)cc3C2=O)CC1]

### (1s,4s)-Methyl 4-(6-(2-(dimethylamino)ethylamino)-4-methyl-1-oxoisoindolin-2-yl)cyclohexanecarboxylate

To a solution of 2,2-diethoxy-N,N-dimethyl-ethanamine (600 mg, 3.72 mmol, 685 mL) in THF (10 mL) was added 10% HCl aq (8.0 mL). The mixture was stirred at 25° C. for 60 minutes. It was concentrated to give 2-(dimethylamino)acetaldehyde (310 mg, crude) as a yellow oil. To a solution of methyl 4-(6-amino-4-methyl-1-oxo-isoindolin-2-yl)cyclohexanecarboxylate (360 mg, 1.19 mmol) and 2-(dimethylamino)acetaldehyde (311 mg, 3.57 mmol) in MeOH (15 mL) was added sodium cyanoborohydride (374 mg, 5.95 mmol). The mixture was stirred at 25° C. for 3 hours. The reaction solution was mixed with saturated sodium carbonate aqueous solution (40 mL), extracted with EtOAc. The organic extracts were washed with brine, dried by Na₂SO₄, filtered and concentrated to give a crude oil. The crude product was purified by silica gel chromatography to give methyl 4-[6-[2-(dimethylamino)ethylamino]-4-methyl-1-oxo-isoindolin-2-yl]cyclohexanecarboxylate (300 mg, 720 mmol, 61% yield, 90% purity) as a yellow solid. LCMS ESI m/z: 374.0 [M+H]⁺.

### Step 3

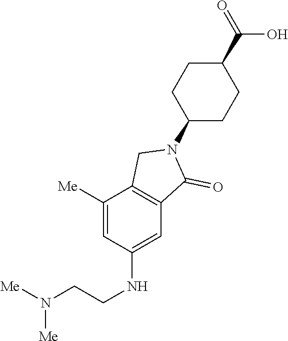 [SMILES: Cc1cc(NCCN(C)C)cc2c1CN([C@H]1CC[C@@H](C(=O)O)CC1)C2=O]

### (1s,4s)-4-(6-(2-(Dimethylamino)ethylamino)-4-methyl-1-oxoisoindolin-2-yl)cyclohexanecarboxylic acid

To a solution of methyl 4-[6-[2-(dimethylamino)ethylamino]-4-methyl-1-oxo-isoindolin-2-yl]cyclohexanecarboxylate (300 mg, 800 mmol) in THF (9 mL)/water (5 mL) was added LiOH (190 mg, 8.0 mmol). The mixture was stirred at 65° C. for 3 hours. The reaction solution was cooled to room temperature, 1 M HCl was added to the reaction mixture, until the solution reached pH~2 to 3. The solution was extracted with EtOAc. The organic extracts were washed with brine, dried by Na₂SO₄, filtered and concentrated to give (1s,4s)-4-(6-(2-(dimethylamino)ethylamino)-4-methyl-1-oxoisoindolin-2-yl)cyclohexanecarboxylic acid (250 mg, crude) as a yellow oil, which was used in the subsequent step without further purification. LCMS ESI m/z: 360.2 [M+H]⁺.

### Step 4

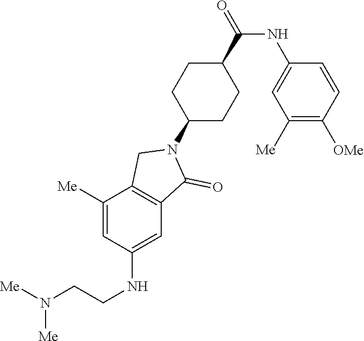 [SMILES: COc1ccc(NC(=O)[C@H]2CC[C@@H](N3Cc4c(C)cc(NCCN(C)C)cc4C3=O)CC2)cc1C]

### (1s,4s)-4-(6-(2-(Dimethylamino)ethylamino)-4-methyl-1-oxoisoindolin-2-yl)-N-(4-methoxy-3-methylphenyl)cyclohexanecarboxamide

To a solution of 4-[6-[2-(dimethylamino)ethylamino]-4-methyl-1-oxo-isoindolin-2-yl]cyclohexanecarboxylic acid (300 mg, 760 mmol, hydrochloride salt) and HATU (350 mg, 910 mmol) in DMF (8 mL) was added DIPEA (290 mg, 2.3 mmol, 400 μL), 30 minutes later, 4-methoxy-3-methyl-aniline (156 mg, 1.14 mmol) was added. The mixture was stirred at 25° C. for 2 hours. The mixture was poured into water and extracted with EtOAc. The organic extracts were washed with brine, dried by Na₂SO₄, filtered and concentrated to give a crude oil. The crude product was purified by silica gel chromatography to give a yellow solid (120 mg, 80% purity). The solid was purified by prep-HPLC to give (1s,4s)-4-(6-(2-(dimethylamino)ethylamino)-4-methyl-1-oxoisoindolin-2-yl)-N-(4-methoxy-3-methylphenyl)cyclohexanecarboxamide (30 mg, 63 mmol, 8.3% yield) as a yellow solid. LCMS ESI m/z: 478.9 [M+H]⁺. ¹H NMR (400 MHz, DMSO-d₆) δ 9.55 (s, 1H), 7.44-7.35 (m, 2H), 6.85 (d, J=8.8 Hz, 1H), 6.66-6.59 (m, 2H), 5.51 (t, J=5.2 Hz, 1H), 4.19 (s, 2H), 4.09-4.05 (m, 1H), 3.74 (s, 3H), 3.14-3.07 (m, 2H), 2.65-2.63 (m, 1H), 2.43 (t, J=6.6 Hz, 2H), 2.21 (s, 3H), 2.18 (s, 6H), 2.12-2.09 (m, 5H), 1.99-1.94 (m, 2H), 1.73-1.67 (m, 2H), 1.61-1.53 (m, 2H).

### Example 257

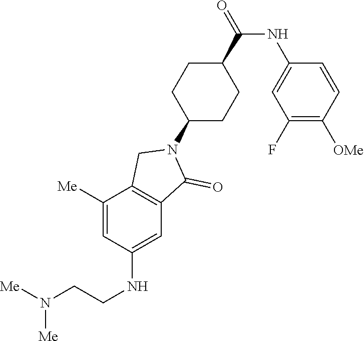 [SMILES: COc1ccc(NC(=O)[C@H]2CC[C@@H](N3Cc4c(C)cc(NCCN(C)C)cc4C3=O)CC2)cc1F]

### (1s,4s)-4-(6-(2-(Dimethylamino)ethylamino)-4-methyl-1-oxoisoindolin-2-yl)-N-(3-fluoro-4-methoxyphenyl)cyclohexanecarboxamide

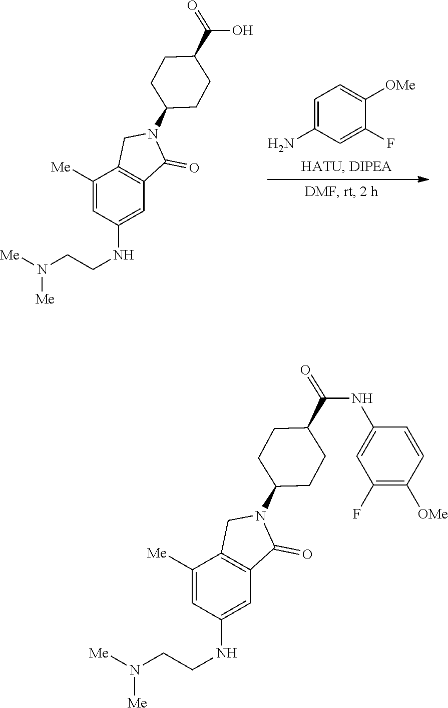 [SMILES: Cc1cc(NCCN(C)C)cc2c1CN([C@H]1CC[C@@H](C(=O)O)CC1)C2=O ; COc1ccc(N)cc1F ; COc1ccc(NC(=O)[C@H]2CC[C@@H](N3Cc4c(C)cc(NCCN(C)C)cc4C3=O)CC2)cc1F]

### Step 1

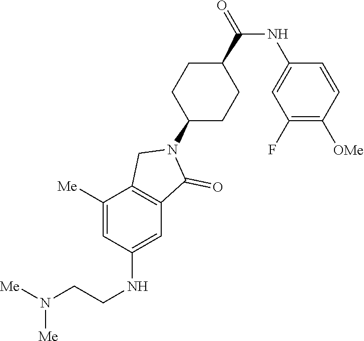 [SMILES: COc1ccc(NC(=O)[C@H]2CC[C@@H](N3Cc4c(C)cc(NCCN(C)C)cc4C3=O)CC2)cc1F]

### (1s,4s)-4-(6-(2-(Dimethylamino)ethylamino)-4-methyl-1-oxoisoindolin-2-yl)-N-(3-fluoro-4-methoxyphenyl)cyclohexanecarboxamide

Following the synthetic procedure for Example 256, but starting with 3-fluoro-4-methoxyaniline gave the title compound as a white solid. LC/MS ESI 482.8 [M+H]⁺. ¹H NMR (400 MHz, DMSO-d₆) δ 9.80 (s, 1H), 7.64 (dd, J=13.9, 2.4 Hz, 1H), 7.28 (dd, J=8.9, 1.5 Hz, 1H), 7.10 (t, J=9.3 Hz, 1H), 6.65-6.60 (m, 2H), 5.51 (t, J=5.5 Hz, 1H), 4.19 (s, 2H), 4.11-4.03 (m, 1H), 3.80 (s, 3H), 3.10 (dd, J=12.3, 6.4 Hz, 2H), 2.66-2.63 (m, 1H), 2.42 (t, J=6.6 Hz, 2H), 2.21 (s, 3H), 2.17 (s, 6H), 2.13-2.09 (m, 2H), 2.00-1.89 (m, 2H), 1.74-1.68 (m, 2H), 1.60-1.56 (m, 2H).

### Example 258

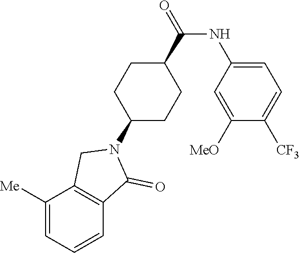 [SMILES: COc1cc(NC(=O)[C@H]2CC[C@@H](N3Cc4c(C)cccc4C3=O)CC2)ccc1C(F)(F)F]

### (1s,4s)-N-(3-Methoxy-4-(trifluoromethyl)phenyl)-4-(4-methyl-1-oxoisoindolin-2-yl)cyclohexanecarboxamide

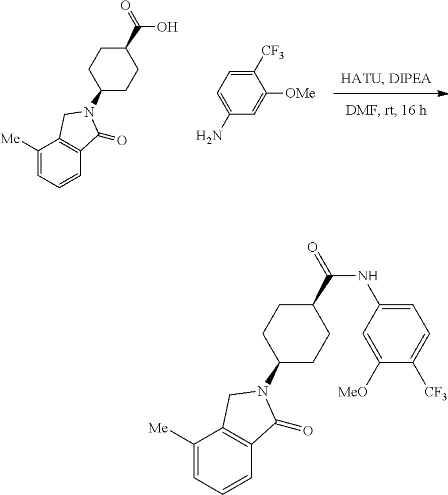 [SMILES: Cc1cccc2c1CN([C@H]1CC[C@@H](C(=O)O)CC1)C2=O ; COc1cc(N)ccc1C(F)(F)F ; COc1cc(NC(=O)[C@H]2CC[C@@H](N3Cc4c(C)cccc4C3=O)CC2)ccc1C(F)(F)F]

### Step 1

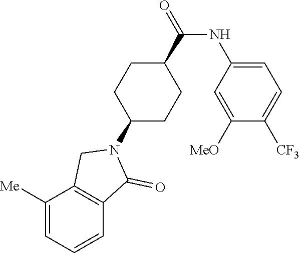 [SMILES: COc1cc(NC(=O)[C@H]2CC[C@@H](N3Cc4c(C)cccc4C3=O)CC2)ccc1C(F)(F)F]

### (1s,4s)-N-(3-Methoxy-4-(trifluoromethyl)phenyl)-4-(4-methyl-1-oxoisoindolin-2-yl)cyclohexanecarboxamide

Following the synthetic procedure for Example 215, but starting with 3-methoxy-4-(trifluoromethyl) aniline gave the title compound as a white solid. LC/MS 447.2 [M+H]⁺. ¹H NMR (400 MHz, DMSO-d₆) δ10.14 (s, 1H), 7.61 (s, 1H), 7.54-7.52 (m, 1H), 7.50-7.45 (m, 1H), 7.40-7.34 (m, 3H), 4.39 (s, 2H), 4.18-4.07 (m, 1H), 3.86 (s, 3H), 2.73-2.72 (m, 1H), 2.34 (s, 3H), 2.17-2.14 (m, 2H), 2.00-1.94 (m, 2H), 1.76-1.73 (m, 2H), 1.64-1.61 (m, 2H).

### Example 259

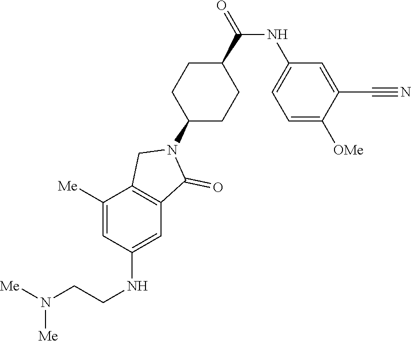 [SMILES: COc1ccc(NC(=O)[C@H]2CC[C@@H](N3Cc4c(C)cc(NCCN(C)C)cc4C3=O)CC2)cc1C#N]

### (1s,4s)-N-(3-Cyano-4-methoxyphenyl)-4-(6-(2-(dimethylamino)ethylamino)-4-methyl-1-oxoisoindolin-2-yl)cyclohexanecarboxamide

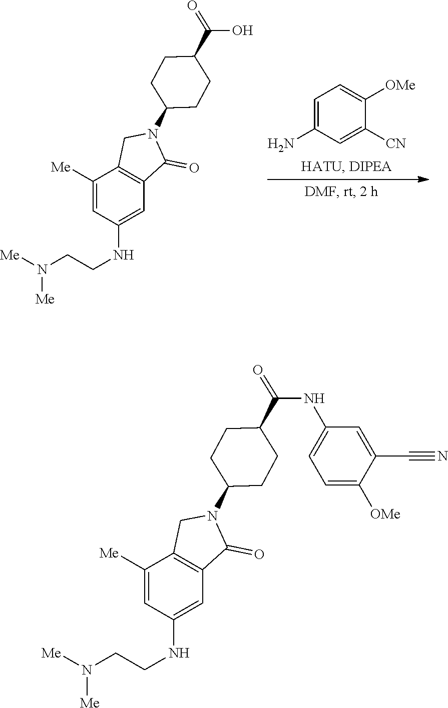 [SMILES: Cc1cc(NCCN(C)C)cc2c1CN([C@H]1CC[C@@H](C(=O)O)CC1)C2=O ; COc1ccc(N)cc1C#N ; COc1ccc(NC(=O)[C@H]2CC[C@@H](N3Cc4c(C)cc(NCCN(C)C)cc4C3=O)CC2)cc1C#N]

### Step 1

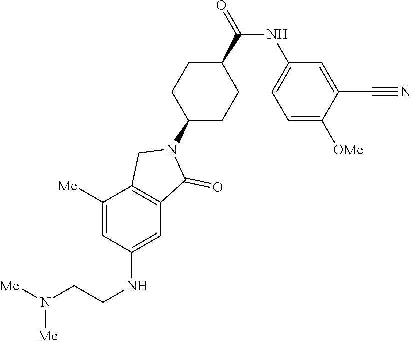 [SMILES: COc1ccc(NC(=O)[C@H]2CC[C@@H](N3Cc4c(C)cc(NCCN(C)C)cc4C3=O)CC2)cc1C#N]

### (1s,4s)-N-(3-Cyano-4-methoxyphenyl)-4-(6-(2-(dimethylamino)ethylamino)-4-methyl-1-oxoisoindolin-2-yl)cyclohexanecarboxamide

Following the synthetic procedure for Example 256, but starting with 5-amino-2-methoxybenzonitrile gave the title compound as a white solid. LC/MS ESI 490.3 [M+H]⁺. ¹H NMR (400 MHz, DMSO-d₆) δ 9.94 (s, 1H), 8.03 (d, J=2.6 Hz, 1H), 7.78 (dd, J=9.2, 2.6 Hz, 1H), 7.22 (d, J=9.2 Hz, 1H), 6.65-6.58 (m, 2H), 5.51 (t, J=5.5 Hz, 1H), 4.19 (s, 2H), 4.10-4.04 (m, 1H), 3.88 (s, 3H), 3.10 (dd, J=12.3, 6.4 Hz, 2H), 2.68-2.66 (m, 1H), 2.42 (t, J=6.6 Hz, 2H), 2.21 (s, 3H), 2.17 (s, 6H), 2.14-2.10 (m, 2H), 1.98-1.89 (m, 2H), 1.72-1.69 (m, 2H), 1.60-1.56 (m, 2H).

### Example 260

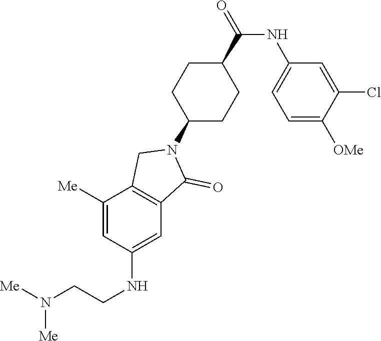 [SMILES: COc1ccc(NC(=O)[C@H]2CC[C@@H](N3Cc4c(C)cc(NCCN(C)C)cc4C3=O)CC2)cc1Cl]

### (1s,4s)-N-(3-Chloro-4-methoxyphenyl)-4-(6-(2-(dimethylamino)ethylamino)-4-methyl-1-oxoisoindolin-2-yl)cyclohexanecarboxamide

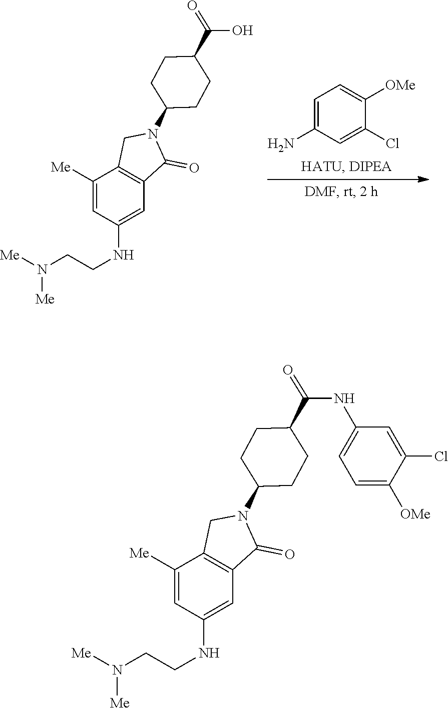 [SMILES: Cc1cc(NCCN(C)C)cc2c1CN([C@H]1CC[C@@H](C(=O)O)CC1)C2=O ; COc1ccc(N)cc1Cl ; COc1ccc(NC(=O)[C@H]2CC[C@@H](N3Cc4c(C)cc(NCCN(C)C)cc4C3=O)CC2)cc1Cl]

### Step 1

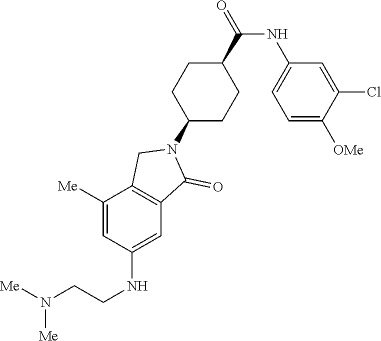 [SMILES: COc1ccc(NC(=O)[C@H]2CC[C@@H](N3Cc4c(C)cc(NCCN(C)C)cc4C3=O)CC2)cc1Cl]

### (1s,4s)-N-(3-Chloro-4-methoxyphenyl)-4-(6-(2-(dimethylamino)ethylamino)-4-methyl-1-oxoisoindolin-2-yl)cyclohexanecarboxamide

Following the synthetic procedure for Example 256, but starting with 3-chloro-4-methoxyaniline gave the title compound as a white solid. LC/MS ESI 499.2 [M+H]⁺. ¹H NMR (400 MHz, DMSO-d₆) δ 9.79 (s, 1H), 7.85 (d, J=2.5 Hz, 1H), 7.45 (dd, J=8.9, 2.5 Hz, 1H), 7.10 (d, J=9.0 Hz, 1H), 6.62 (d, J=11.3 Hz, 2H), 5.51 (t, J=5.5 Hz, 1H), 4.19 (s, 2H), 4.08-4.06 (m, 1H), 3.81 (s, 3H), 3.11-3.08 (m, 2H), 2.66-2.64 (m, 1H), 2.42 (t, J=6.6 Hz, 2H), 2.21 (s, 3H), 2.17 (s, 6H), 2.12-2.09 (m, 2H), 1.97-1.93 (m, 2H), 1.72-1.60 (m, 2H), 1.59-1.56 (m, 2H).

### Example 261

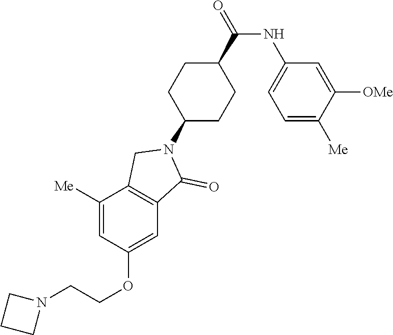 [SMILES: COc1cc(NC(=O)[C@H]2CC[C@@H](N3Cc4c(C)cc(OCCN5CCC5)cc4C3=O)CC2)ccc1C]

### (1s,4s)-4-(6-(2-(Azetidin-1-yl) ethoxy)-4-methyl-1-oxoisoindolin-2-yl)-N-(3-methoxy-4-methylphenyl)cyclohexanecarboxamide

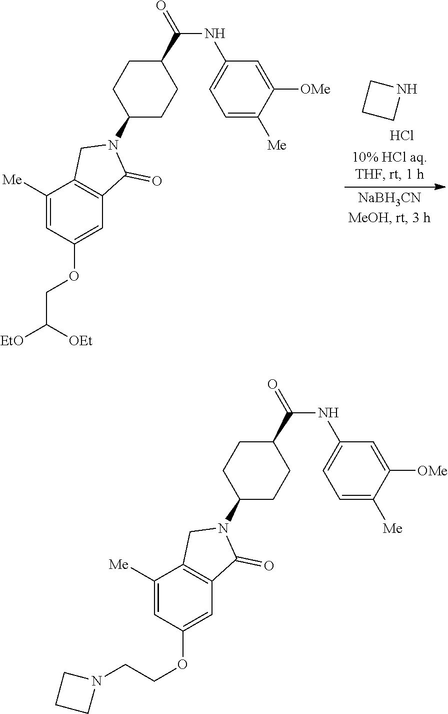

### Step 1

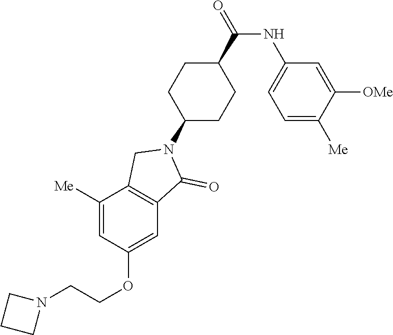 [SMILES: COc1cc(NC(=O)[C@H]2CC[C@@H](N3Cc4c(C)cc(OCCN5CCC5)cc4C3=O)CC2)ccc1C]

### (1s,4s)-4-(6-(2-(Azetidin-1-yl) ethoxy)-4-methyl-1-oxoisoindolin-2-yl)-N-(3-methoxy-4-methylphenyl)cyclohexanecarboxamide

Following the synthetic procedure for Example 255, but starting with azetidine hydrochloride gave the title compound as a white solid. LC/MS ESI 491.9 [M+H]⁺. ¹H NMR (400 MHz, DMSO-d₆) δ 9.70 (s, 1H), 7.32 (d, J=1.6 Hz, 1H), 7.12 (dd, J=8.0, 1.8 Hz, 1H), 7.03-6.93 (m, 3H), 4.29 (s, 2H), 4.12-4.08 (m, 1H), 3.94 (t, J=5.6 Hz, 2H), 3.76 (s, 3H), 3.17 (t, J=6.9 Hz, 4H), 2.70-2.66 (m, 3H), 2.29 (s, 3H), 2.12-2.05 (m, 5H), 2.01-1.93 (m, 4H), 1.75-1.70 (m, 2H), 1.62-1.57 (m, 2H).

### Example 263

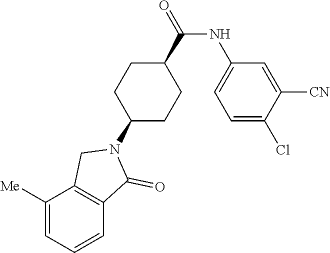 [SMILES: Cc1cccc2c1CN([C@H]1CC[C@@H](C(=O)Nc3ccc(Cl)c(C#N)c3)CC1)C2=O]

### (1s,4s)-N-(4-Chloro-3-cyanophenyl)-4-(4-methyl-1-oxoisoindolin-2-yl)cyclohexanecarboxamide

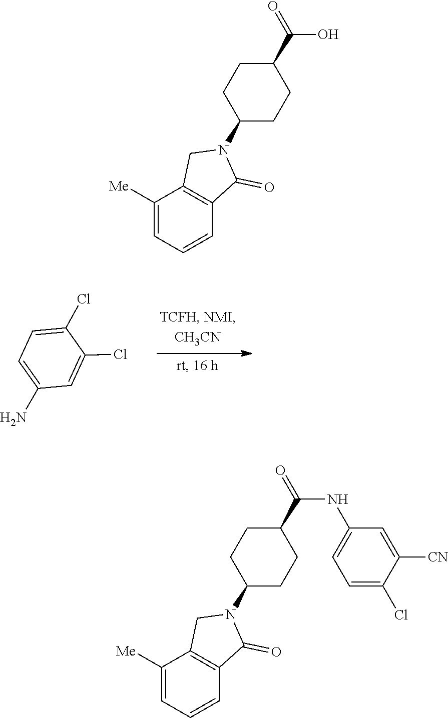 [SMILES: Cc1cccc2c1CN([C@H]1CC[C@@H](C(=O)O)CC1)C2=O ; Nc1ccc(Cl)c(Cl)c1 ; Cc1cccc2c1CN([C@H]1CC[C@@H](C(=O)Nc3ccc(Cl)c(C#N)c3)CC1)C2=O]

### Step 1

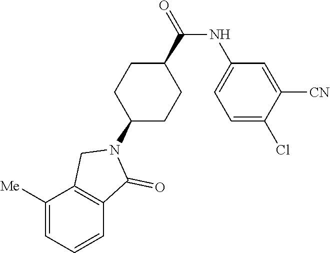 [SMILES: Cc1cccc2c1CN([C@H]1CC[C@@H](C(=O)Nc3ccc(Cl)c(C#N)c3)CC1)C2=O]

### (1s,4s)-N-(4-Chloro-3-cyanophenyl)-4-(4-methyl-1-oxoisoindolin-2-yl)cyclohexanecarboxamide

To a solution of 4-(4-methyl-1-oxo-isoindolin-2-yl)cyclohexanecarboxylic acid (0.10 g, 370 mmol) and 5-amino-2-chloro-benzonitrile (73 mg, 480 mmol) and 1-methylimidazole (90 mg, 1.1 mmol, 87 μL) in CH₃CN (10 mL) was added [chloro(dimethylamino)-methylene]-dimethyl-ammonium; hexafluorophosphate (120 mg, 440 mmol) and then stirred at 25° C. for 16 hours. The reaction mixture was poured into H₂O (50 mL) and extracted with EtOAc (3×50 mL). The combined organic extracts were dried over Na₂SO₄ and evaporated in vacuo. The mixture was recrystallization by EtOH (3 mL) and CH₃CN (3 mL) to give (1s,4s)-N-(4-chloro-3-cyanophenyl)-4-(4-methyl-1-oxoisoindolin-2-yl)cyclohexanecarboxamide (0.023 g, 56 mmol, 15% yield) as a white solid. LCMS ESI m/z: 407.8 [M+H]⁺. ¹H NMR (400 MHz, DMSO-d₆) δ 10.26 (s, 1H), 8.28 (d, J=2.4 Hz, 1H), 7.90-7.80 (m, 1H), 7.71-7.65 (m, 1H), 7.54-7.44 (m, 1H), 7.40-7.35 (m, 2H), 4.38 (s, 2H), 4.18-4.08 (m, 1H), 2.76-2.70 (m, 1H), 2.34 (s, 3H), 2.20-2.10 (m, 2H), 2.03-1.89 (m, 2H), 1.80-1.70 (m, 2H), 1.67-1.57 (m, 2H).

### Example 264

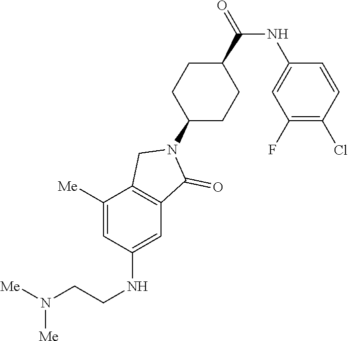 [SMILES: Cc1cc(NCCN(C)C)cc2c1CN([C@H]1CC[C@@H](C(=O)Nc3ccc(Cl)c(F)c3)CC1)C2=O]

### (1s,4s)-N-(4-Chloro-3-fluorophenyl)-4-(6-(2-(dimethylamino)ethylamino)-4-methyl-1-oxoisoindolin-2-yl)cyclohexanecarboxamide

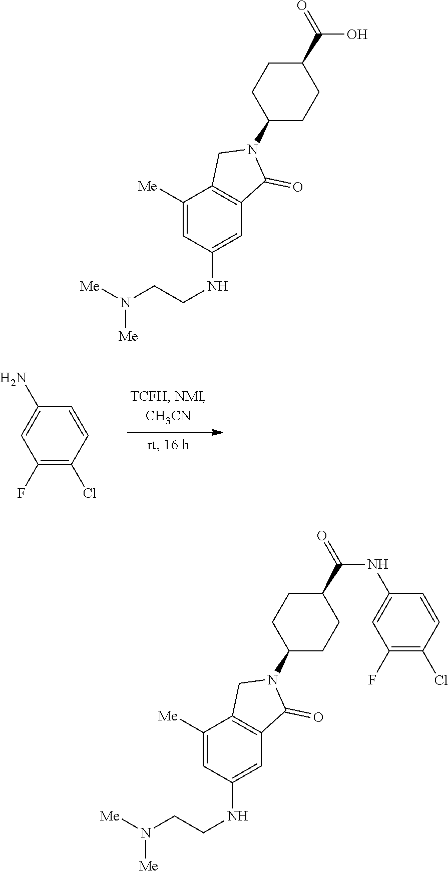 [SMILES: Cc1cc(NCCN(C)C)cc2c1CN([C@H]1CC[C@@H](C(=O)O)CC1)C2=O ; Nc1ccc(Cl)c(F)c1 ; Cc1cc(NCCN(C)C)cc2c1CN([C@H]1CC[C@@H](C(=O)Nc3ccc(Cl)c(F)c3)CC1)C2=O]

### Step 1

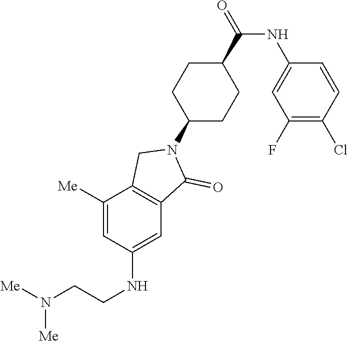 [SMILES: Cc1cc(NCCN(C)C)cc2c1CN([C@H]1CC[C@@H](C(=O)Nc3ccc(Cl)c(F)c3)CC1)C2=O]

### (1s,4s)-N-(4-Chloro-3-fluorophenyl)-4-(6-(2-(dimethylamino)ethylamino)-4-methyl-1-oxoisoindolin-2-yl)cyclohexanecarboxamide

Following the synthetic procedure for Example 263, but starting with (1s,4s)-4-(6-(2-(dimethylamino)ethylamino)-4-methyl-1-oxoisoindolin-2-yl)cyclohexanecarboxylic acid and 4-chloro-3-fluoroaniline gave the title compound as a white solid. LC/MS ESI 487.2 [M+H]⁺. 1H-NMR (400 MHz, DMSO-d₆) δ 10.09 (s, 1H), 7.79 (dd, J=12.1, 2.3 Hz, 1H), 7.45 (t, J=8.7 Hz, 1H), 7.33 (dd, J=8.8, 1.6 Hz, 1H), 6.61-6.54 (m, 2H), 5.46 (t, J=5.4 Hz, 1H), 4.14 (s, 2H), 4.04-4.01 (m, 1H), 3.05 (dd, J=12.2, 6.3 Hz, 2H), 2.66-2.64 (m, 1H), 2.37 (t, J=6.6 Hz, 2H), 2.15 (s, 3H), 2.12-2.0 (m, 6H), 2.07-2.04 (m, 2H), 1.92-1.82 (m, 2H), 1.70-1.67 (m, 2H), 1.55-1.52 (m, 2H).

### Example 265

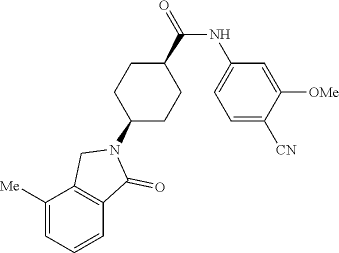 [SMILES: COc1cc(NC(=O)[C@H]2CC[C@@H](N3Cc4c(C)cccc4C3=O)CC2)ccc1C#N]

### (1s,4s)-N-(4-Cyano-3-methoxyphenyl)-4-(4-methyl-1-oxoisoindolin-2-yl)cyclohexanecarboxamide

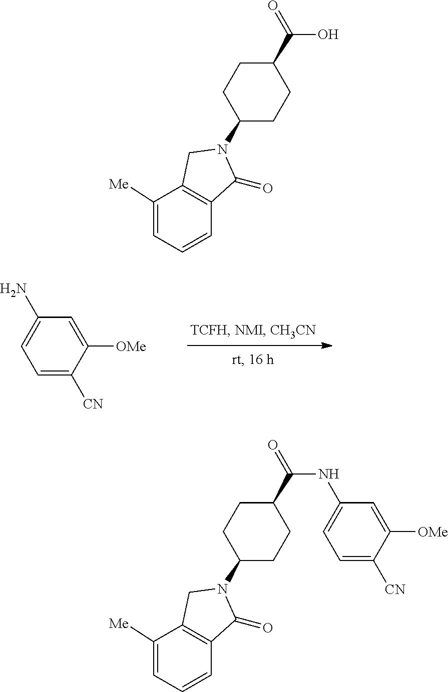 [SMILES: Cc1cccc2c1CN([C@H]1CC[C@@H](C(=O)O)CC1)C2=O ; COc1cc(N)ccc1C#N ; COc1cc(NC(=O)[C@H]2CC[C@@H](N3Cc4c(C)cccc4C3=O)CC2)ccc1C#N]

### Step 1

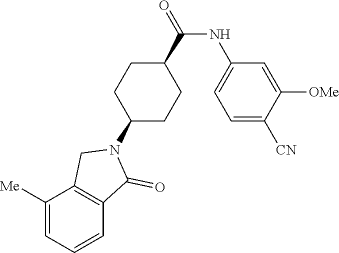 [SMILES: COc1cc(NC(=O)[C@H]2CC[C@@H](N3Cc4c(C)cccc4C3=O)CC2)ccc1C#N]

### (1s,4s)-N-(4-Cyano-3-methoxyphenyl)-4-(4-methyl-1-oxoisoindolin-2-yl)cyclohexanecarboxamide

Following the synthetic procedure for Example 263, but starting with 4-amino-2-methoxybenzonitrile gave the title compound as a light-yellow solid. LC/MS ESI 404.2 [M+H]⁺. ¹H NMR (400 MHz, DMSO-d₆) δ 10.24 (s, 1H), 7.64-7.62 (m, 2H), 7.51-7.45 (m, 1H), 7.41-7.32 (m, 3H), 4.39 (s, 2H), 4.15-4.10 (m, 1H), 3.89 (s, 3H), 2.75-2.73 (m, 1H), 2.34 (s, 3H), 2.16-2.13 (m, 2H), 2.04-1.91 (m, 2H), 1.80-1.73 (m, 2H), 1.64-1.62 (m, 2H).

### Example 266

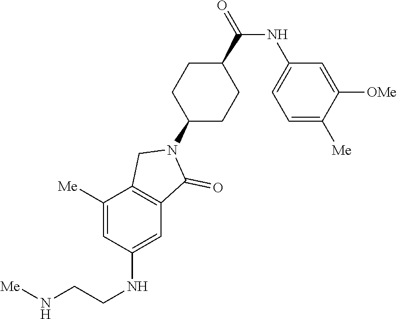 [SMILES: CNCCNc1cc(C)c2c(c1)C(=O)N([C@H]1CC[C@@H](C(=O)Nc3ccc(C)c(OC)c3)CC1)C2]

### (1s,4s)-N-(3-Methoxy-4-methylphenyl)-4-(4-methyl-6-(2-(methylamino)ethoxy)-1-oxoisoindolin-2-yl)cyclohexanecarboxamide

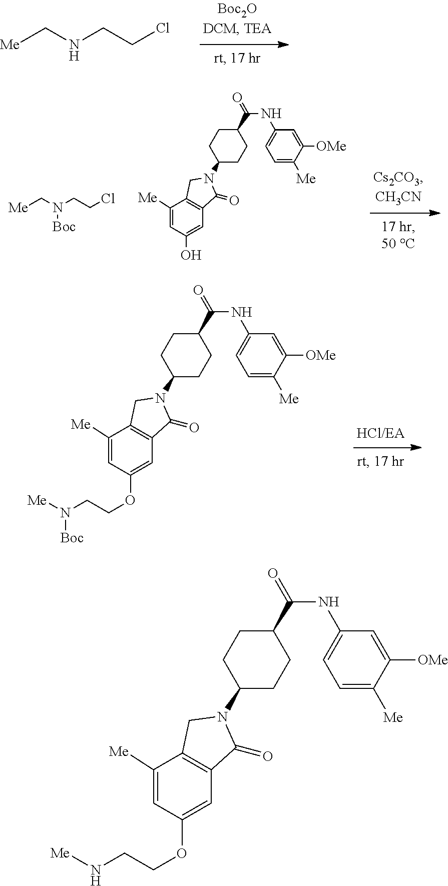 [SMILES: CCNCCCl ; CCN(CCCl)C(=O)OC(C)(C)C ; COc1cc(NC(=O)[C@H]2CC[C@@H](N3Cc4c(C)cc(O)cc4C3=O)CC2)ccc1C ; COc1cc(NC(=O)[C@H]2CC[C@@H](N3Cc4c(C)cc(OCCN(C)C(=O)OC(C)(C)C)cc4C3=O)CC2)ccc1C ; CNCCOc1cc(C)c2c(c1)C(=O)N([C@H]1CC[C@@H](C(=O)Nc3ccc(C)c(OC)c3)CC1)C2]

### Step 1

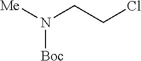 [SMILES: CN(CCCl)C(=O)OC(C)(C)C]

### tert-Butyl 2-chloroethyl(methyl)carbamate

To a solution of 2-chloro-N-methyl-ethanamine (1.00 g, 7.69 mmol, hydrochloride salt) in DCM (11 mL) at 25° C. was added TEA (1.56 g, 15.4 mmol, 2.14 mL) and Boc₂O (2.0 g, 9.2 mmol). The mixture was stirred for 1 hour at 25° C. To the reaction mixture was added EtOAc (50 mL) and washed with H₂O (3×50 mL). The combined organic extracts were dried over Na₂SO₄ and evaporated in vacuo. The residue was purified by flash chromatography on silica gel (0-10% EtOAc in PE) to get to get tert-butyl 2-chloroethyl(methyl)carbamate (1.2 g, 6.4 mmol, 83% yield) as a colorless oil. ¹HNMR (400 MHz, CDCl₃) δ 3.61-3.51 (m, 4H), 2.94 (s, 3H), 1.47 (s, 9H).

### Step 2

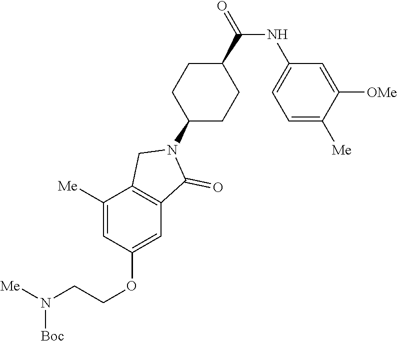 [SMILES: COc1cc(NC(=O)[C@H]2CC[C@@H](N3Cc4c(C)cc(OCCN(C)C(=O)OC(C)(C)C)cc4C3=O)CC2)ccc1C]

### tert-Butyl 2-(2-((1s,4s)-4-(3-methoxy-4-methylphenylcarbamoyl)cyclohexyl)-7-methyl-3-oxoisoindolin-5-yloxy)ethyl(methyl)carbamate

To a solution of 4-(6-hydroxy-4-methyl-1-oxo-isoindolin-2-yl)-N-(3-m ethoxy-4-methyl-phenyl)cyclohexanecarboxamide (100 mg, 240 mmol) in MeCN (10 mL) was added tert-butyl N-(2-chloroethyl)-N-methyl-carbamate (47 mg, 240 mmol) and Cs₂CO₃ (160 mg, 490 mmol). The mixture was stirred at 50° C. for 17 hours. The mixture was filtered and concentrated in vacuum. The residue was purified by prep-TLC to give tert-butyl(2-((2-((1s,4s)-4-((3-methoxy-4-methylphenyl)carbamoyl)cyclohexyl)-7-methyl-3-oxoisoindolin-5-yl)oxy)ethyl)(methyl)carbamate was obtained (70 mg, 200.4 mmol) as a yellow solid. LCMS ESI m/z 509.9 [M−56+H]⁺.

### Step 3

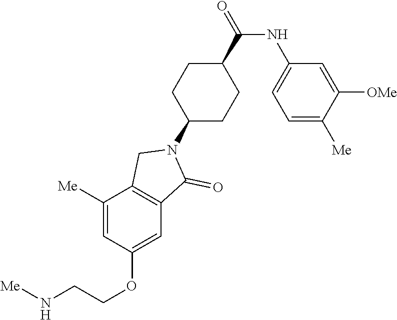 [SMILES: CNCCOc1cc(C)c2c(c1)C(=O)N([C@H]1CC[C@@H](C(=O)Nc3ccc(C)c(OC)c3)CC1)C2]

### (1s,4s)-N-(3-Methoxy-4-methylphenyl)-4-(4-methyl-6-(2-(methylamino)ethoxy)-1-oxoisoindolin-2-yl)cyclohexanecarboxamide

A solution of tert-butyl N-[2-[2-[4-[(3-methoxy-4-methyl-phenyl)carbamoyl]cyclohexyl]-7-methyl-3-oxo-isoindolin-5-yl]oxyethyl]-N-methyl-carbamate (120 mg, 210 mmol) in HCl/EtOAc (4M, 5 mL) was stirred at 25° C. for 17 hours. The mixture was concentrated and purified by prep-HPLC to give (1s,4s)-N-(3-methoxy-4-methylphenyl)-4-(4-methyl-6-(2-(methylamino)ethoxy)-1-oxoisoindolin-2-yl)cyclohexanecarboxamide (25 mg, 54 mmol, 25.31% yield) as a white solid. LCMS ESI m/z 466.3 [M+H]⁺. ¹H NMR (400 MHz, DMSO-d₆) δ 9.71 (s, 1H), 7.32 (d, J=1.7 Hz, 1H), 7.12 (dd, J=8.1, 1.8 Hz, 1H), 7.06-6.92 (m, 3H), 4.30 (s, 2H), 4.15-4.02 (m, 3H), 3.76 (s, 3H), 2.85 (t, J=5.5 Hz, 2H), 2.68-2.66 (m, 1H), 2.35 (s, 3H), 2.29 (s, 3H), 2.19-2.05 (m, 5H), 2.01-1.92 (m, 2H), 1.76-1.69 (m, 2H), 1.65-1.53 (m, 2H).

### Example 267, Example 268, Example 269 and Example 270

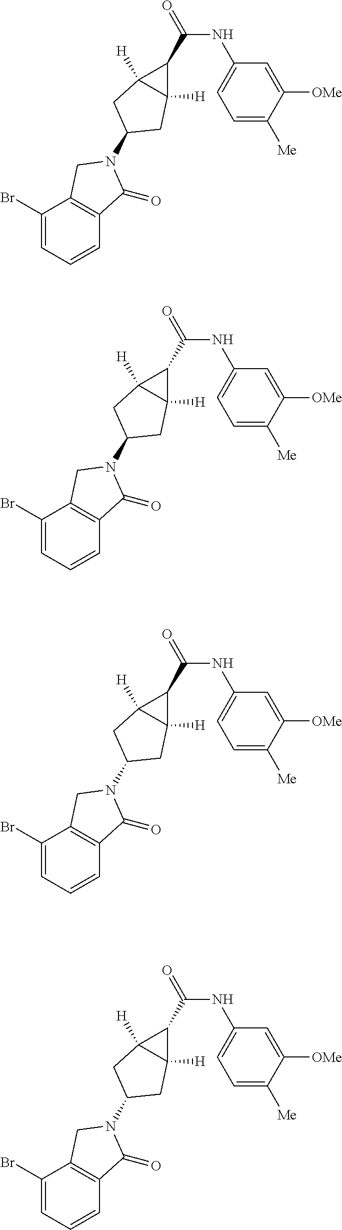 [SMILES: COc1cc(NC(=O)[C@@H]2[C@@H]3C[C@H](N4Cc5c(Br)cccc5C4=O)C[C@@H]32)ccc1C ; COc1cc(NC(=O)[C@H]2[C@@H]3C[C@H](N4Cc5c(Br)cccc5C4=O)C[C@@H]32)ccc1C ; COc1cc(NC(=O)[C@@H]2[C@@H]3C[C@@H](N4Cc5c(Br)cccc5C4=O)C[C@@H]32)ccc1C ; COc1cc(NC(=O)[C@H]2[C@@H]3C[C@@H](N4Cc5c(Br)cccc5C4=O)C[C@@H]32)ccc1C]

### (1R,3r,5S,6s)-3-(4-Bromo-1-oxoisoindolin-2-yl)-N-(3-methoxy-4-methylphenyl)bicyclo[3.1.0]hexane-6-carboxamide, (1R,3r,5S,6r)-3-(4-Bromo-1-oxoisoindolin-2-yl)-N-(3-methoxy-4-methylphenyl)bicyclo[3.1.0]hexane-6-carboxamide, (1R,3s,5S,6s)-3-(4-Bromo-1-oxoisoindolin-2-yl)-N-(3-methoxy-4-methylphenyl)bicyclo[3.1.0]hexane-6-carboxamide and (1R,3s,5S,6r)-3-(4-Bromo-1-oxoisoindolin-2-yl)-N-(3-methoxy-4-methylphenyl)bicyclo[3.1.0]hexane-6-carboxamide

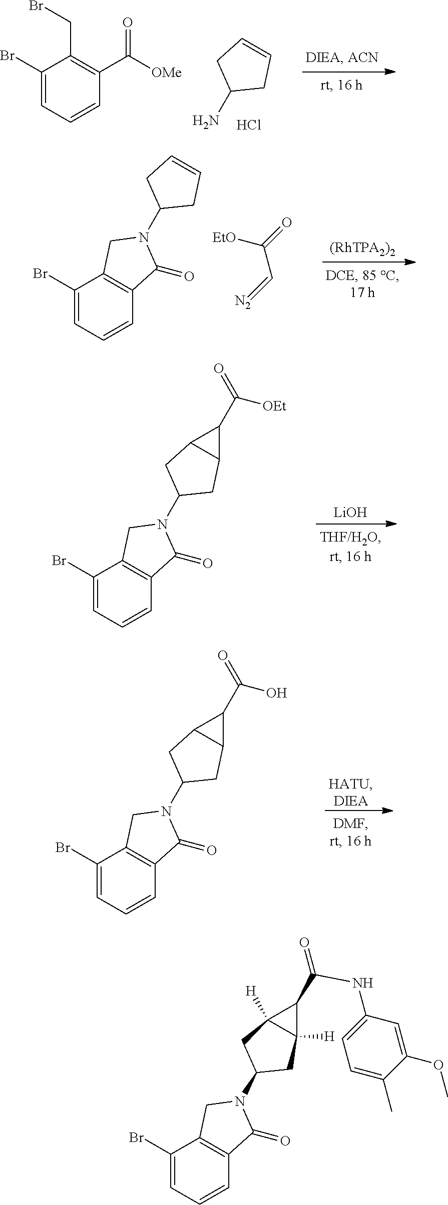

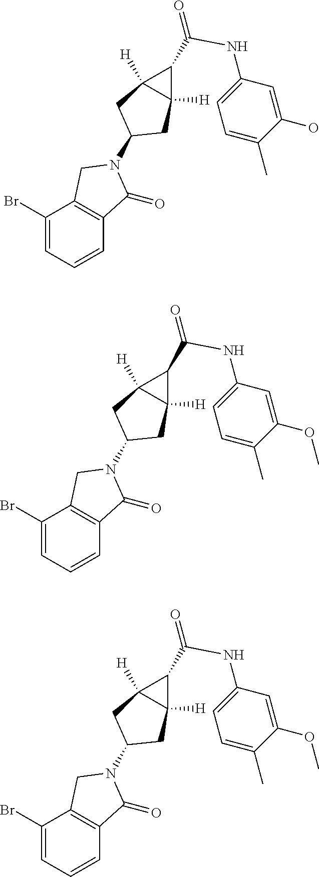 [SMILES: Cc1ccc(NC(=O)[C@H]2[C@@H]3C[C@H](N4Cc5c(Br)cccc5C4=O)C[C@@H]32)cc1O ; COc1cc(NC(=O)[C@@H]2[C@@H]3C[C@@H](N4Cc5c(Br)cccc5C4=O)C[C@@H]32)ccc1C ; COc1cc(NC(=O)[C@H]2[C@@H]3C[C@@H](N4Cc5c(Br)cccc5C4=O)C[C@@H]32)ccc1C]

### Step 1

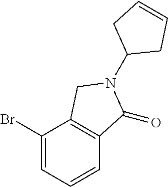 [SMILES: O=C1c2cccc(Br)c2CN1C1CC=CC1]

### 4-Bromo-2-(cyclopent-3-enyl)isoindolin-1-one

A solution of methyl 3-bromo-2-(bromomethyl)benzoate (7.00 g, 22.6 mmol), cyclopent-3-enamine hydrochloride (2.70 g, 22.6 mmol) and DIPEA (8.7 g, 68 mmol) in MeCN (30 mL) was stirred at room temperature for 16 hours. The reaction mixture was poured into water (30 mL) and extracted with EtOAc (40 mL). The organic layer was dried and concentrated. The residue was purified by silica gel chromatography (PE/EtOAc=40/1 to 20/1) to give 4-bromo-2-(cyclopent-3-enyl)isoindolin-1-one (3.8 g, 61% yield) as a white solid. LCMS ESI m/z 280.0 [M+H]⁺.

### Step 2

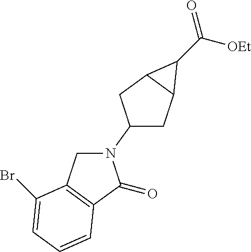 [SMILES: CCOC(=O)C1C2CC(N3Cc4c(Br)cccc4C3=O)CC21]

### Ethyl 3-(4-bromo-1-oxoisoindolin-2-yl)bicyclo[3.1.0]hexane-6-carboxylate

A solution 4-bromo-2-cyclopent-3-en-1-yl-isoindolin-1-one (700 mg, 2.52 mmol) and (RhTPA₂)₂ (68.2 mg, 50.3 mmol) in 1,2-dichloroethane (20 mL) was heated to 85° C. under N₂. Ethyl 2-diazoacetate (1.44 g, 12.6 mmol) was added dropwise to the reaction mixture, and the mixture was stirred at 85° C. for 17 hours under N₂. The mixture was concentrated to the crude, which was purified by silica column (PE/EtOAc=40/1 to 10/1) to give ethyl 3-(4-bromo-1-oxoisoindolin-2-yl)bicyclo[3.1.0]hexane-6-carboxylate (700 mg, 1.9 mmol, 76% yield) as a white solid. LCMS ESI m/z 366.0 [M+H]⁺

### Step 3

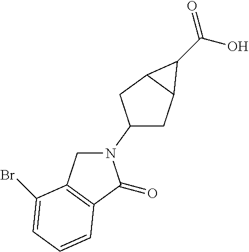 [SMILES: O=C(O)C1C2CC(N3Cc4c(Br)cccc4C3=O)CC21]

### 3-(4-Bromo-1-oxoisoindolin-2-yl)bicyclo[3.1.0]hexane-6-carboxylic acid

To a solution of ethyl 3-(4-bromo-1-oxo-isoindolin-2-yl)bicycle [3.1.0]hexane-6-carboxylate (700 mg, 1.60 mmol) in THF (10 mL) and H₂O (10 mL) was added LiOH (410 mg, 17.9 mmol). The mixture was stirred at 25° C. for 17 hours. 1 M HCl was added to the reaction mixture until the solution reached PH~2. The solution was extracted with EtOAc (30 mL*3). The organic phases were concentrated in vacuum to give 3-(4-bromo-1-oxoisoindolin-2-yl)bicyclo[3.1.0]hexane-6-carboxylic acid (500 mg, 1.5 mmol, 94% yield) as a yellow solid. LCMS ESI m/z 335.7 [M+H]⁺

### Step 4

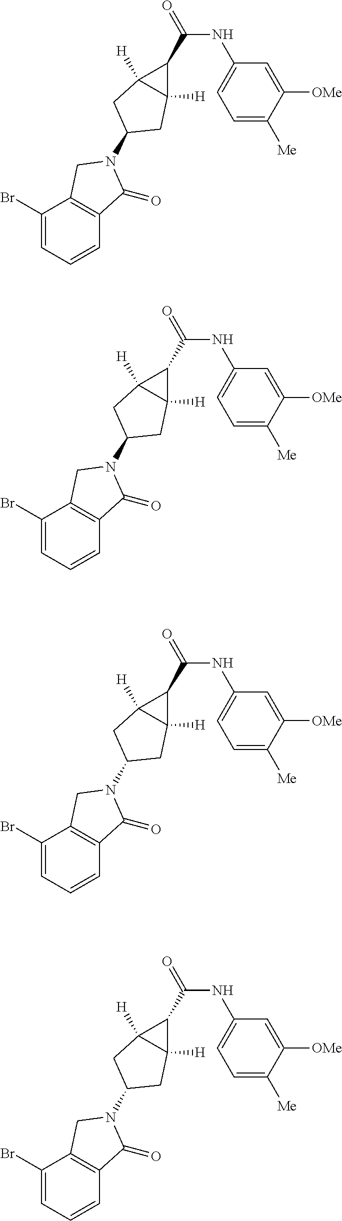 [SMILES: COc1cc(NC(=O)[C@@H]2[C@@H]3C[C@H](N4Cc5c(Br)cccc5C4=O)C[C@@H]32)ccc1C ; COc1cc(NC(=O)[C@H]2[C@@H]3C[C@H](N4Cc5c(Br)cccc5C4=O)C[C@@H]32)ccc1C ; COc1cc(NC(=O)[C@@H]2[C@@H]3C[C@@H](N4Cc5c(Br)cccc5C4=O)C[C@@H]32)ccc1C ; COc1cc(NC(=O)[C@H]2[C@@H]3C[C@@H](N4Cc5c(Br)cccc5C4=O)C[C@@H]32)ccc1C]

### (1R,3r,5S,6s)-3-(4-Bromo-1-oxoisoindolin-2-yl)-N-(3-methoxy-4-methylphenyl)bicyclo[3.1.0]hexane-6-carboxamide, (1R,3r,5S,6r)-3-(4-Bromo-1-oxoisoindolin-2-yl)-N-(3-methoxy-4-methylphenyl)bicyclo[3.1.0]hexane-6-carboxamide, (1R,3s,5S,6s)-3-(4-Bromo-1-oxoisoindolin-2-yl)-N-(3-methoxy-4-methylphenyl)bicyclo[3.1.0]hexane-6-carboxamide and (1R,3s,5S,6r)-3-(4-Bromo-1-oxoisoindolin-2-yl)-N-(3-methoxy-4-methylphenyl)bicyclo[3.1.0]hexane-6-carboxamide

To a solution of 3-(4-bromo-1-oxo-isoindolin-2-yl)bicyclo[3.1.0]hexane-6-carboxylic acid (600 mg, 1.78 mmol) in DMF (10 mL) was added HATU (814.34 mg, 2.14 mmol), TEA (541.80 mg, 5.35 mmol, 746.28 μL) and 3-methoxy-4-methyl-aniline (269.31 mg, 1.96 mmol). The mixture was stirred at 25° C. for 17 hr. The mixture was poured into EtOAc (50 mL) and washed with water (30 mL*3). The organic layer was dried and concentrated. The residue was purified by Pre-TLC and prep-HPLC to give (1R,3r,5S,6s)-3-(4-bromo-1-oxoisoindolin-2-yl)-N-(3-methoxy-4-methylphenyl)bicyclo[3.1.0]hexane-6-carboxamide (Example 267) (28 mg, 61.4 μmol, 3.4% yield) as a yellow solid, (1R,3r,5S,6r)-3-(4-bromo-1-oxoisoindolin-2-yl)-N-(3-methoxy-4-methylphenyl)bicyclo[3.1.0]hexane-6-carboxamide (Example 268) (20 mg, 43.92 μmol, 2.46% yield) as a white solid, (1R,3s,5S,6s)-3-(4-bromo-1-oxoisoindolin-2-yl)-N-(3-methoxy-4-methylphenyl)bicyclo[3.1.0]hexane-6-carboxamide (Example 269) (28 mg, 43.9 μmol, 3.4% yield) as a white solid and (1R,3s,5S,6r)-3-(4-bromo-1-oxoisoindolin-2-yl)-N-(3-methoxy-4-methylphenyl)bicyclo[3.1.0]hexane-6-carboxamide (Example 270) (14 mg, 30.7 μmol, 1.7% yield) as a white solid. These compounds were racemic and stereochemistry was arbitrarily assigned.

Example 267: LCMS ESI m/z 454.7 [M+H]⁺. ¹H NMR (400 MHz, DMSO-d₆) δ 10.02 (s, 1H), 7.81 (d, J=7.4 Hz, 1H), 7.69 (d, J=7.3 Hz, 1H), 7.47 (t, J=7.7 Hz, 1H), 7.31 (d, J=1.2 Hz, 1H), 7.03-6.93 (m, 2H), 4.51-4.29 (m, 3H), 3.73 (s, 3H), 2.18-2.12 (m, 2H), 2.07 (s, 3H), 2.05-1.96 (m, 2H), 1.92-1.88 (m, 1H), 1.83 (s, 2H).

Example 268: LCMS ESI m/z 454.7 [M+H]⁺. ¹H NMR (400 MHz, DMSO-d₆) δ 10.13 (s, 1H), 7.88-7.76 (m, 1H), 7.69 (d, J=7.4 Hz, 1H), 7.47 (t, J=7.7 Hz, 1H), 7.30 (s, 1H), 7.00 (s, 2H), 4.88-4.84 (m, 1H), 4.38 (s, 2H), 3.73 (s, 3H), 2.42-2.32 (m, 2H), 2.07 (s, 3H), 1.92-1.68 (m, 5H).

Example 269: LCMS ESI m/z 454.7 [M+H]⁺. ¹H NMR (400 MHz, DMSO-d₆) δ 9.94 (s, 1H), 7.79 (dd, J=7.9, 0.7 Hz, 1H), 7.71-7.59 (m, 1H), 7.44 (t, J=7.7 Hz, 1H), 7.31 (d, J=1.5 Hz, 1H), 7.10 (dd, J=8.0, 1.7 Hz, 1H), 7.02 (d, J=8.1 Hz, 1H), 4.81-4.74 (m, 1H), 4.44 (s, 2H), 3.77 (s, 3H), 2.29-2.05 (m, 7H), 1.92-1.81 (m, 2H), 1.80-1.70 (m, 1H).

Example 270: LCMS ESI m/z 454.7 [M+H]⁺. ¹H NMR (400 MHz, DMSO-d₆) δ 10.19 (s, 1H), 7.80 (d, J=7.8 Hz, 1H), 7.68 (d, J=7.4 Hz, 1H), 7.53 (s, 1H), 7.45 (t, J=7.7 Hz, 1H), 7.06-6.95 (m, 2H), 5.16-4.95 (m, 1H), 4.51 (s, 2H), 3.77 (s, 3H), 2.27-2.16 m, 2H), 2.11-2.04 (m, 5H), 1.89 (s, 3H).

### Example 271

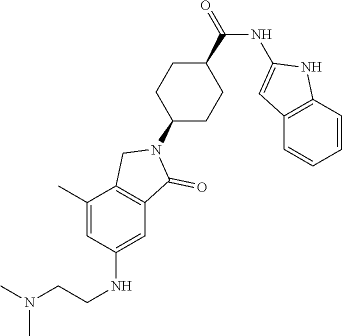 [SMILES: Cc1cc(NCCN(C)C)cc2c1CN([C@H]1CC[C@@H](C(=O)Nc3cc4ccccc4[nH]3)CC1)C2=O]

### (1s,4s)-4-(6-(2-(dimethylamino)ethylamino)-4-methyl-1-oxoisoindolin-2-yl)-N-(1H-indol-2-yl)cyclohexanecarboxamide

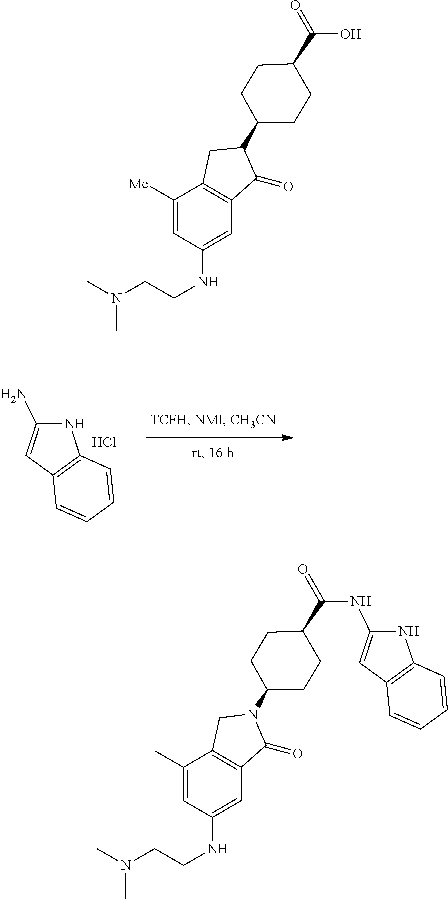 [SMILES: Cc1cc(NCCN(C)C)cc2c1CC([C@H]1CC[C@@H](C(=O)O)CC1)C2=O ; Nc1cc2ccccc2[nH]1 ; Cl ; Cc1cc(NCCN(C)C)cc2c1CN([C@H]1CC[C@@H](C(=O)Nc3cc4ccccc4[nH]3)CC1)C2=O]

### Step 1

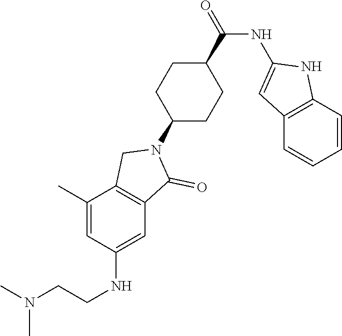 [SMILES: Cc1cc(NCCN(C)C)cc2c1CN([C@H]1CC[C@@H](C(=O)Nc3cc4ccccc4[nH]3)CC1)C2=O]

### (1s,4s)-4-(6-(2-(dimethylamino)ethylamino)-4-methyl-1-oxoisoindolin-2-yl)-N-(1H-indol-2-yl)cyclohexanecarboxamide

To a solution of (1s,4s)-4-(6-(2-(dimethylamino)ethylamino)-4-methyl-1-oxoisoindolin-2-yl)cyclohexanecarboxylic acid (100 mg, 278.19 μmol), 1H-indol-2-amine (44.12 mg, 261.65 μmol, HCl) in MeCN (5 mL) was added TCFH (93.63 mg, 333.83 μmol) and NMI (45.62 mg, 556.38 μmol). The mixture was stirred at rt overnight. The solution was poured into water (20 mL) and extracted with EtOAc (20 mL). The organic layer was dried and concentrated. The residue was purified by Prep-HPLC to give (1s,4s)-4-(6-(2-(dimethylamino)ethylamino)-4-methyl-1-oxoisoindolin-2-yl)-N-(1H-indol-2-yl)cyclohexanecarboxamide (40 mg, 84.46 μmol, 30.3% yield) as a white solid. LC/MS ESI 473.9 [M+H]⁺. ¹H NMR (400 MHz, DMSO-d₆) δ 11.02 (s, 1H), 10.56 (s, 1H), 7.42-7.37 (m, 1H), 7.35-7.32 (m, 1H), 6.95-6.87 (m, 2H), 6.62 (d, J=6.8 Hz, 2H), 6.00 (d, J=1.5 Hz, 1H), 5.53 (b, 1H), 4.17 (s, 2H), 4.12-4.09 (m, 1H), 3.12-3.10 (m, 2H), 2.75-2.73 (m, 1H), 2.47-2.44 (m, 2H), 2.19-2.14 (m, 11H), 2.00-1.91 (m, 2H), 1.74 (t, J=13.2 Hz, 2H), 2.14-1.98 (m, 2H).

### Example 272

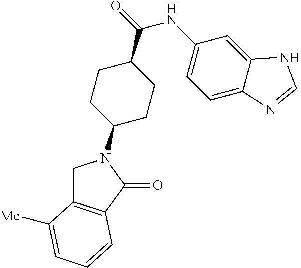 [SMILES: Cc1cccc2c1CN([C@H]1CC[C@@H](C(=O)Nc3ccc4nc[nH]c4c3)CC1)C2=O]

### (1s,4s)-N-(1H-benzo[d]imidazol-6-yl)-4-(4-methyl-1-oxoisoindolin-2-yl)cyclohexanecarboxamide

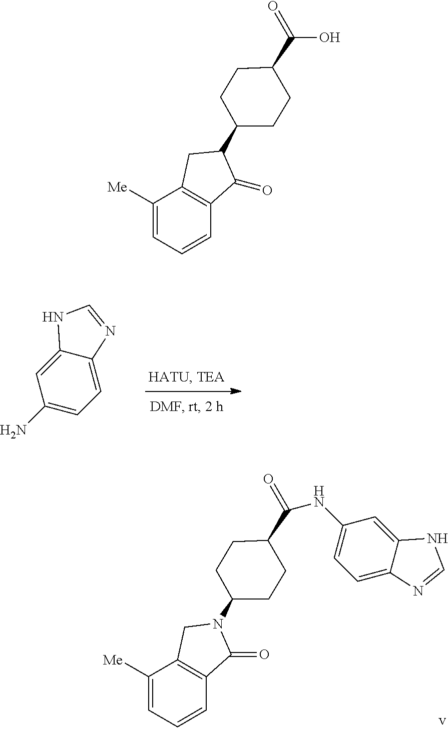 [SMILES: Cc1cccc2c1CC([C@H]1CC[C@@H](C(=O)O)CC1)C2=O ; Nc1ccc2nc[nH]c2c1 ; Cc1cccc2c1CN([C@H]1CC[C@@H](C(=O)Nc3ccc4nc[nH]c4c3)CC1)C2=O]

### Step 1

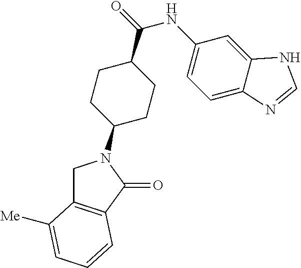 [SMILES: Cc1cccc2c1CN([C@H]1CC[C@@H](C(=O)Nc3ccc4nc[nH]c4c3)CC1)C2=O]

### (1s,4s)-N-(1H-benzo[d]imidazol-6-yl)-4-(4-methyl-1-oxoisoindolin-2-yl)cyclohexanecarboxamide

To a solution of 4-(4-methyl-1-oxo-isoindolin-2-yl)cyclohexanecarboxylic acid (0.06 g, 219.52 μmol), HATU (91.81 mg, 241.47 μmol) and TEA (44.43 mg, 439.04 μmol, 61.19 μL) in DMF (2.99 mL) was added 3H-benzimidazol-5-amine (32.15 mg, 241.47 μmol). The mixture was stirred for 1 h at 25° C. To the reaction mixture was added H₂O (50 mL) and extracted with EtOAc (3×50 mL). The combined organic extracts were dried over Na₂SO₄ and evaporated in vacuo. The residue was purified by Prep-HPLC to get (1s,4s)-N-(1H-benzo[d]imidazol-6-yl)-4-(4-methyl-1-oxoisoindolin-2-yl)cyclohexanecarboxamide (0.0177 g, 45.56 μmol, 20.76% yield) as a white solid. LCMS ESI m/z: 389.2 [M+H]⁺. ¹H NMR (400 MHz, DMSO-d₆) δ 12.32 (br, 1H), 9.78 (s, 1H), 8.13 (s, 1H), 8.08 (s, 1H), 7.55-7.47 (m, 2H), 7.40-7.35 (m, 2H), 7.33-7.27 (m, 1H), 4.38 (s, 2H), 4.21-4.04 (m, 1H), 2.72-2.70 (m, 1H), 2.33 (s, 3H), 2.22-2.12 (m, 2H), 2.10-2.00 (m, 2H), 1.80-1.70 (m, 2H), 1.65-1.52 (m, 2H).

### Example 273

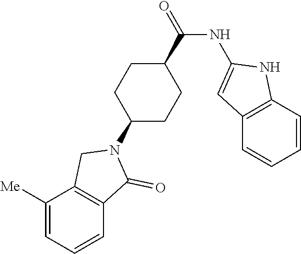 [SMILES: Cc1cccc2c1CN([C@H]1CC[C@@H](C(=O)Nc3cc4ccccc4[nH]3)CC1)C2=O]

### (1s,4s)-N-(1H-indol-2-yl)-4-(4-methyl-1-oxoisoindolin-2-yl)cyclohexanecarboxamide

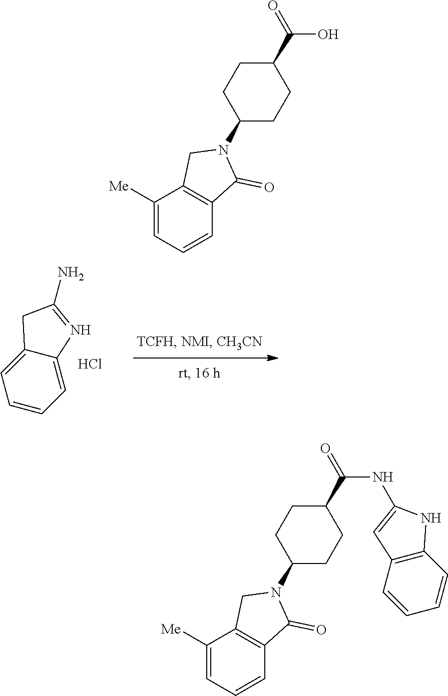 [SMILES: Cc1cccc2c1CN([C@H]1CC[C@@H](C(=O)O)CC1)C2=O ; NC1=Nc2ccccc2C1 ; Cl ; Cc1cccc2c1CN([C@H]1CC[C@@H](C(=O)Nc3cc4ccccc4[nH]3)CC1)C2=O]

### Step 1

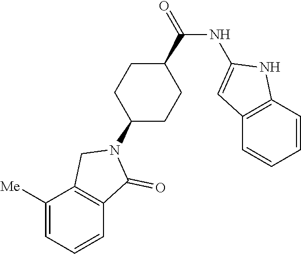 [SMILES: Cc1cccc2c1CN([C@H]1CC[C@@H](C(=O)Nc3cc4ccccc4[nH]3)CC1)C2=O]

### (1s,4s)-N-(1H-indol-2-yl)-4-(4-methyl-1-oxoisoindolin-2-yl)cyclohexanecarboxamide

To a solution of (1s,4s)-4-(4-methyl-1-oxoisoindolin-2-yl)cyclohexanecarboxylic acid (50 mg, 182.93 μmol), 1H-indol-2-amine (29.01 mg, 172.05 μmol, HCl) in MeCN (5 mL), was added 1-Methylimidazole (52.57 mg, 640.26 μmol, 50.98 μL) and TCFH (61.59 mg, 219.51 mmol). The reaction mixture was stirred at rt overnight. The solution poured into water (20 mL) and extracted with EtOAc (30 mL). The organic layer was washed with brine, dried by Na₂SO₄ and concentrated. The residue was purified by Prep-HPLC to give (1s,4s)-N-(1H-indol-2-yl)-4-(4-methyl-1-oxoisoindolin-2-yl)cyclohexanecarboxamide (25 mg, 64.52 μmol, 35.2% yield) as a white solid. LC/MS ESI 387.8 [M+H]⁺. ¹H NMR (400 MHz, DMSO-d₆) δ 11.03 (s, 1H), 10.57 (s, 1H), 7.51-7.46 (m, 1H), 7.43-7.39 (m, 1H), 7.39-7.33 (m, 3H), 6.98-6.89 (m, 2H), 6.02 (d, J=1.5 Hz, 1H), 4.37 (s, 2H), 4.18-4.12 (m, 1H), 2.78-2.76 (m, 1H), 2.33 (s, 3H), 2.20-2.17 (m, 2H), 2.07-1.95 (m, 2H), 1.80-1.74 (m, 2H), 1.66-1.64 (m, 2H).

### Example 274

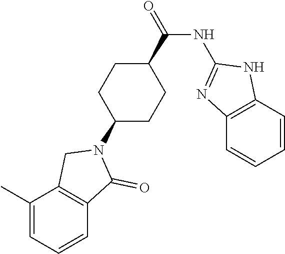 [SMILES: Cc1cccc2c1CN([C@H]1CC[C@@H](C(=O)Nc3nc4ccccc4[nH]3)CC1)C2=O]

### (1s,4s)-N-(1H-benzo[d]imidazol-2-yl)-4-(4-methyl-1-oxoisoindolin-2-yl)cyclohexanecarboxamide

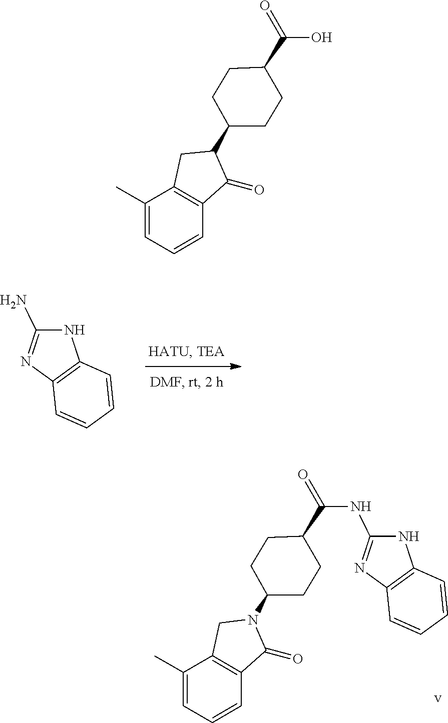 [SMILES: Cc1cccc2c1CC([C@H]1CC[C@@H](C(=O)O)CC1)C2=O ; Nc1nc2ccccc2[nH]1 ; Cc1cccc2c1CN([C@H]1CC[C@@H](C(=O)Nc3nc4ccccc4[nH]3)CC1)C2=O]

### Step 1

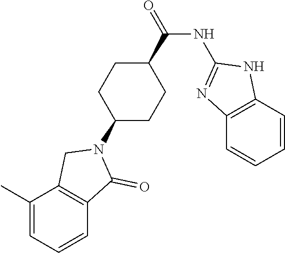 [SMILES: Cc1cccc2c1CN([C@H]1CC[C@@H](C(=O)Nc3nc4ccccc4[nH]3)CC1)C2=O]

### (1s,4s)-N-(1H-benzo[d]imidazol-2-yl)-4-(4-methyl-1-oxoisoindolin-2-yl)cyclohexanecarboxamide

Following the general procedure above in Example 272, but starting with 1H-benzo[d]imidazol-2-amine gave the title compound as a light-yellow solid. LC/MS ESI 389.2 [M+H]⁺. ¹H NMR (400 MHz, DMSO-d₆) δ 12.12 (br, 1H), 11.47 (br, 1H), 7.53-7.33 (m, 5H), 7.11-7.04 (m, 2H), 4.36 (s, 2H), 4.19-4.11 (m, 1H), 2.87 (s, 1H), 2.32 (s, 3H), 2.21-2.16 (m, 2H), 2.11-1.95 (m, 2H), 1.85-1.71 (m, 2H), 1.71-1.59 (m, 2H).

### Example 275

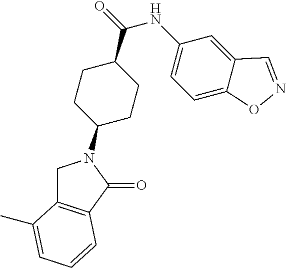 [SMILES: Cc1cccc2c1CN([C@H]1CC[C@@H](C(=O)Nc3ccc4oncc4c3)CC1)C2=O]

### (1s,4s)-N-(benzo[d]isoxazol-5-yl)-4-(4-methyl-1-oxoisoindolin-2-yl)cyclohexanecarboxamide

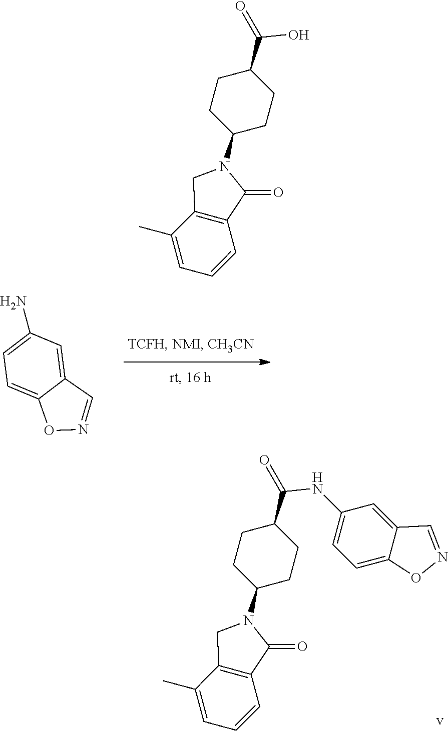 [SMILES: Cc1cccc2c1CN([C@H]1CC[C@@H](C(=O)O)CC1)C2=O ; Nc1ccc2oncc2c1 ; Cc1cccc2c1CN([C@H]1CC[C@@H](C(=O)Nc3ccc4oncc4c3)CC1)C2=O]

### Step 1

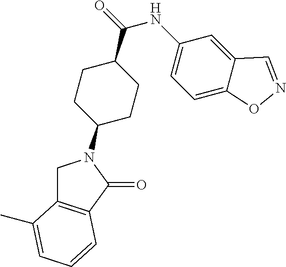 [SMILES: Cc1cccc2c1CN([C@H]1CC[C@@H](C(=O)Nc3ccc4oncc4c3)CC1)C2=O]

### (1s,4s)-N-(benzo[d]isoxazol-5-yl)-4-(4-methyl-1-oxoisoindolin-2-yl)cyclohexanecarboxamide

Following the general procedure above in Example 273, but starting with benzo[d]isoxazol-5-amine gave the title compound as a white solid. LC/MS ESI 389.9 [M+H]⁺. ¹H NMR (400 MHz, DMSO-d₆) δ 9.80 (s, 1H), 7.91 (s, 1H), 7.57 (dd, J=9.0, 2.6 Hz, 1H), 7.50-7.45 (m, 1H), 7.39-7.33 (m, 2H), 6.94 (d, J=9.0 Hz, 1H), 4.38 (s, 2H), 4.15-4.07 (m, 1H), 2.66 (s, 1H), 2.34 (s, 3H), 2.16-2.09 (m, 2H), 2.02-1.95 (m, 2H), 1.75-1.67 (m, 2H), 1.65-1.58 (m, 2H).

### Example 276

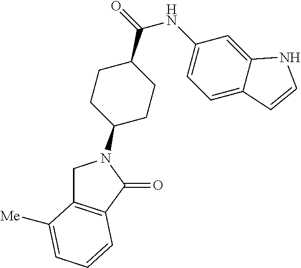 [SMILES: Cc1cccc2c1CN([C@H]1CC[C@@H](C(=O)Nc3ccc4cc[nH]c4c3)CC1)C2=O]

### (1s,4s)-N-(1H-indol-6-yl)-4-(4-methyl-1-oxoisoindolin-2-yl)cyclohexanecarboxamide

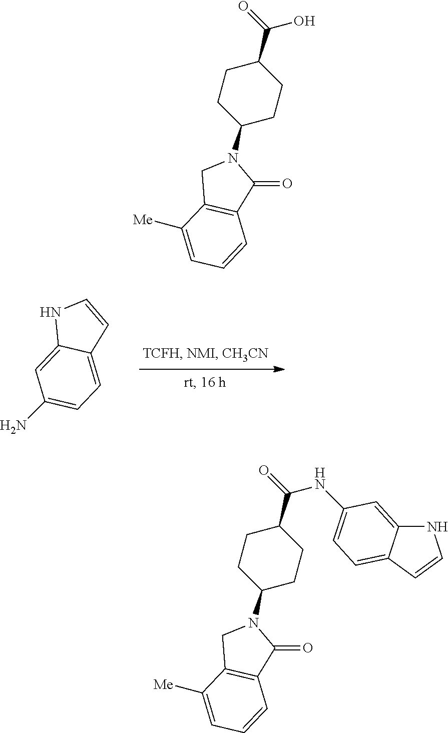 [SMILES: Cc1cccc2c1CN([C@H]1CC[C@@H](C(=O)O)CC1)C2=O ; Nc1ccc2cc[nH]c2c1 ; Cc1cccc2c1CN([C@H]1CC[C@@H](C(=O)Nc3ccc4cc[nH]c4c3)CC1)C2=O]

### Step 1

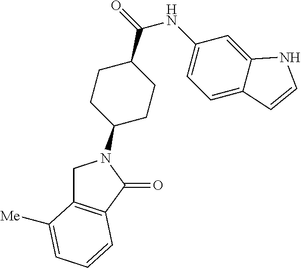 [SMILES: Cc1cccc2c1CN([C@H]1CC[C@@H](C(=O)Nc3ccc4cc[nH]c4c3)CC1)C2=O]

### (1s,4s)-N-(1H-indol-6-yl)-4-(4-methyl-1-oxoisoindolin-2-yl)cyclohexanecarboxamide

Following the general procedure above in Example 273, but starting with 1H-indol-6-amine gave the title compound as a white solid. LC/MS ESI 388.2 [M+H]⁺. ¹H NMR (400 MHz, DMSO-d₆) δ 10.95 (s, 1H), 9.68 (s, 1H), 8.00 (s, 1H), 7.49 (dd, J=5.6, 3.0 Hz, 1H), 7.45-7.31 (m, 3H), 7.27-7.20 (m, 1H), 7.07 (dd, J=8.5, 1.7 Hz, 1H), 6.34 (s, 1H), 4.39 (s, 2H), 4.23-4.05 (m, 1H), 2.73-1.71 (m, 1H), 2.34 (s, 3H), 2.17-1.13 (m, 2H), 2.11-2.02 (m, 2H), 1.78-1.71 (m, 2H), 1.67-1.56 (m, 2H).

### Example 277

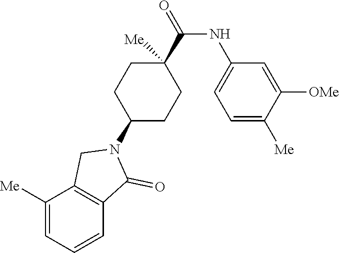 [SMILES: COc1cc(NC(=O)[C@]2(C)CC[C@H](N3Cc4c(C)cccc4C3=O)CC2)ccc1C]

### (1s,4s)-N-(3-methoxy-4-methylphenyl)-1-methyl-4-(4-methyl-1-oxoisoindolin-2-yl)cyclohexane-1-carboxamide

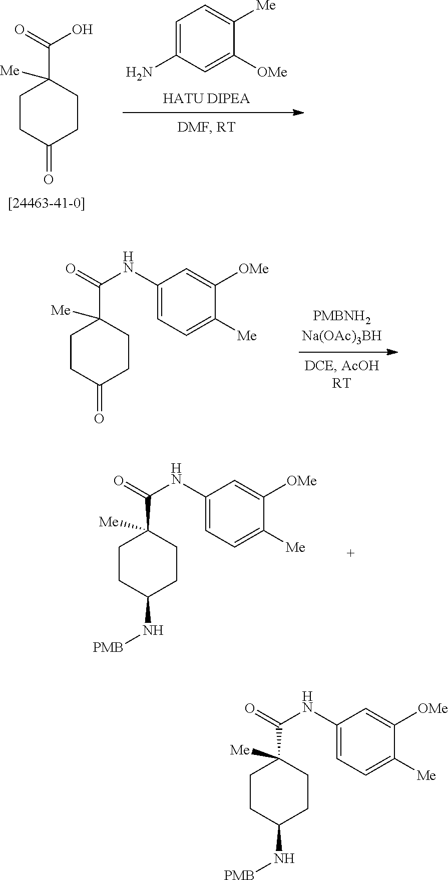

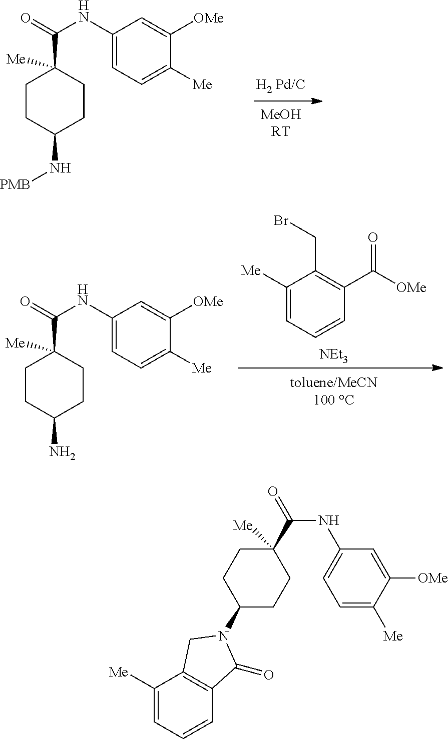

### Step 1

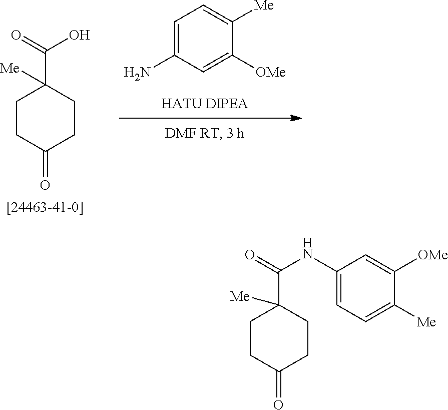 [SMILES: CC1(C(=O)O)CCC(=O)CC1 ; COc1cc(N)ccc1C ; COc1cc(NC(=O)C2(C)CCC(=O)CC2)ccc1C]

### N-(3-methoxy-4-methylphenyl)-1-methyl-4-oxocyclohexane-1-carboxamide

To a solution of 1-methyl-4-oxo-cyclohexanecarboxylic acid (1.10 g, 7.04 mmol), 3-methoxy-4-methyl-aniline (1.06 g, 7.75 mmol) and HATU (2.95 g, 7.75 mmol) dissolved in DMF (6 mL) and cooled by water/ice bath was added N,N-diisopropylethylamine (1.82 g, 14.1 mmol) all at once. The cold bath was removed and reaction stirred to room temperature. After 3 hours at RT LC/MS indicated single peak with mass corresponding to desired product predominated. The reaction solution was partitioned between 50 mL each diethyl ether and water. The aqueous separation was extracted with a second volume of 50 ml diethyl ether. The combined organic layers were washed with 50 ml water, 50 ml 4% aqueous hydrochloric acid and 50 ml saturated aqueous sodium bicarbonate solution then stirred over anhydrous magnesium sulfate, filtered and the filtrate concentrated under reduced pressure to afford N-(3-methoxy-4-methylphenyl)-1-methyl-4-oxocyclohexane-1-carboxamide as a peach-colored foam (1.63 g, 5.93 mmol). The product was used in the next step without further purification. LC/MS ESI 276.3 [M+H]⁺

### Step 2

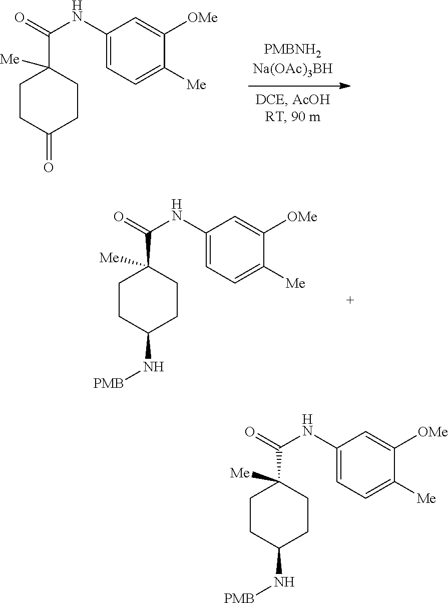

### (1s,4s)-N-(3-Methoxy-4-methylphenyl)-4-((4-methoxybenzyl)amino)-1-methylcyclohexane-1-carboxamide and (1r,4r)-N-(3-methoxy-4-methylphenyl)-4-((4-methoxybenzyl)amino)-1-methylcyclohexane-1-carboxamide

To solution of N-(3-methoxy-4-methyl-phenyl)-1-methyl-4-oxo-cyclohexanecarboxamide (1.63 g, 5.92 mmol) and (4-methoxyphenyl)methanamine (1.02 g, 7.40 mmol) dissolved in 1,2-dichloroethane (40 mL) and acetic acid (2.5 mL) at room temperature was added solid Sodium triacetoxyborohydride (3.2 g, 15 mmol) all at once. The reaction was stirred at ambient temperature for 90 minutes, after which time LCMS indicated single peak with mass corresponding to expected product predominated. The reaction was poured into 50 mL water and to the resulting biphasic mixture was added 2.5 grams sodium hydroxide dissolved in 10 ml water. The biphasic mixture was stirred vigorously for 30 minutes at ambient temperature. The separated organic extract was washed with 50 ml brine then stirred over anhydrous magnesium sulfate, filtered and the filtrate concentrated under reduced pressure to afford a peach-colored viscous oil/glass. The product was isolated by chromatography on ISCO system by dissolving the crude material in minimum volume of DCM and adsorbing onto a 25 gram Biotage Sfar silica gel column then eluting with 2-10% methanol gradient in dichloromethane with 0.5% v/v concentrated aqueous ammonium hydroxide additive and collecting 16×150 mm test tube fractions.

Fractions from Peak 1 were combined and concentrated to afford (1s,4s)-N-(3-methoxy-4-methylphenyl)-4-((4-methoxybenzyl)amino)-1-methylcyclohexane-1-carboxamide as a colorless foam (arbitrarily assigned (1s,4s), 0.29 g, 0.73 mmol). LC/MS ESI 397.2 [M+H]⁺

Fractions from Peak 2 were combined and concentrated to afford (1r,4r)-N-(3-methoxy-4-methylphenyl)-4-((4-methoxybenzyl)amino)-1-methylcyclohexane-1-carboxamide (arbitrarily assigned (1r,4r), 0.38 g, 0.96 mmol) as a colorless foam. LC/MS ESI 397.2 [M+H]⁺

### Step 3

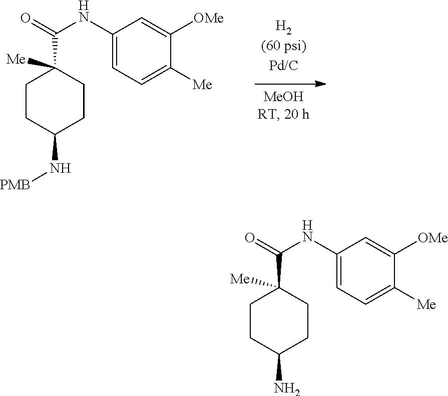

### (1s,4s)-4-amino-N-(3-methoxy-4-methylphenyl)-1-methylcyclohexane-1-carboxamide

A mixture of (1s,4s)-N-(3-methoxy-4-methylphenyl)-4-((4-methoxybenzyl)amino)-1-methylcyclohexane-1-carboxamide (0.29 g, 730 μmol) and palladium (10% on carbon, Type 487, dry, 0.10 g, 940 μmol) in methanol (8 mL) was charged into a septum-capped 20 ml vial with stir bar and purged with nitrogen for five minutes before placing in steel bomb. The septum cap was pierced with a needle and the steel bomb sealed. The sealed bomb was pressurized with hydrogen gas to 60 psig and left to stir under pressure at ambient temperature overnight. After 20 hours LCMS indicated no starting material remained and a single peak with mass corresponding to expected product predominated. The reaction mixture was purged with nitrogen and then filtered through a pad of celite. The filtrate was concentrated under reduced pressure to afford (1s,4s)-4-amino-N-(3-methoxy-4-methylphenyl)-1-methylcyclohexane-1-carboxamide as a colorless solid (0.150 g, 540 μmol) LC/MS ESI 277.3 [M+H]⁺

### Step 4

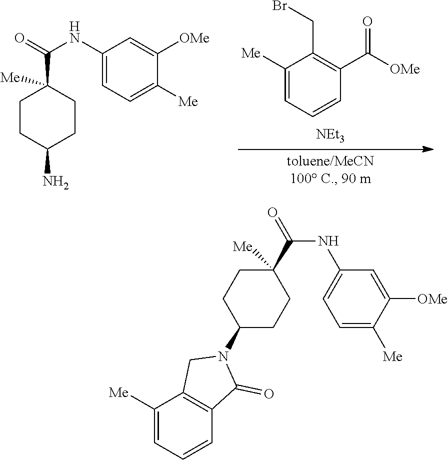 [SMILES: COc1cc(NC(=O)[C@]2(C)CC[C@H](N)CC2)ccc1C ; COC(=O)c1cccc(C)c1CBr ; COc1cc(NC(=O)[C@]2(C)CC[C@H](N3Cc4c(C)cccc4C3=O)CC2)ccc1C]

### (1s,4s)-N-(3-methoxy-4-methylphenyl)-1-methyl-4-(4-methyl-1-oxoisoindolin-2-yl)cyclohexane-1-carboxamide

A glass vial charged with methyl 2-(bromomethyl)-3-methylbenzoate (100 mg, 411 mmol), (1s,4s)-4-amino-N-(3-methoxy-4-methylphenyl)-1-methylcyclohexane-1-carboxamide (125 mg, 452 mmol) and triethylamine (50 mg, 490 mmol) in 3 mL toluene and 1 mL acetonitrile was sealed and heated to 100° C. for ninety minutes, after which time LCMS indicated no ester starting material remained and a peak with mass corresponding to desired product predominated. The reaction was equilibrated to room temperature and partitioned between 25 mL water and 25 mL ethyl acetate. The aqueous separation was extracted again with 25 mL ethyl acetate. The combined organic layers were washed with 25 mL brine, stirred over anhydrous magnesium sulfate, filtered and the filtrate concentrated under reduced pressure to afford crude product as a pink foam. The product was isolated by chromatography on ISCO CombiFlash Companion with a 10 gram Biotage Sfar silica gel column eluting with 25% ethyl acetate in hexane. Fractions containing product were combined and concentrated under reduced pressure to afford (1s,4s)-N-(3-methoxy-4-methylphenyl)-1-methyl-4-(4-methyl-1-oxoisoindolin-2-yl)cyclohexane-1-carboxamide as a colorless foamy solid (96 mg, 240 mmol). LC/MS ESI 407.2 [M+H]⁺. ¹H NMR (400 MHz, CHLOROFORM-d) δ ppm 1.24 (s, 3H) 1.40-1.55 (m, 2H) 1.60-1.84 (m, 4H) 2.11 (s, 3H) 2.23 (s, 3H) 2.28 (br d, J=12.30 Hz, 2H) 3.79 (s, 3H) 4.11 (s, 2H) 4.26 (tt, J=11.80, 4.14 Hz, 1H) 6.78 (dd, J=8.03, 2.01 Hz, 1H) 7.00 (d, J=8.28 Hz, 1H) 7.19-7.25 (m, 1H) 7.25-7.32 (m, 1H) 7.37 (d, J=2.01 Hz, 1H) 7.44 (s, 1H) 7.59 (d, J=7.53 Hz, 1H).

### Example 278

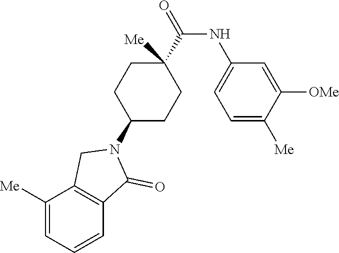 [SMILES: COc1cc(NC(=O)[C@]2(C)CC[C@@H](N3Cc4c(C)cccc4C3=O)CC2)ccc1C]

### (1r,4r)-N-(3-methoxy-4-methylphenyl)-1-methyl-4-(4-methyl-1-oxoisoindolin-2-yl)cyclohexane-1-carboxamide

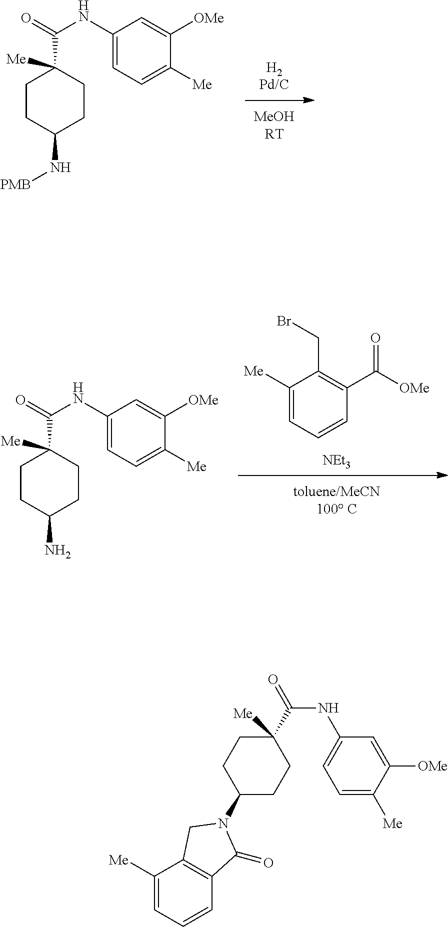

### Step 1

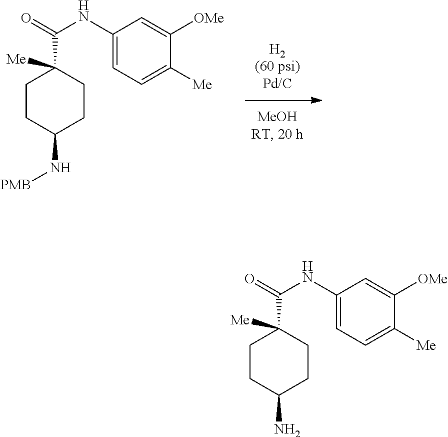

### (1r,4r)-4-amino-N-(3-methoxy-4-methylphenyl)-1-methylcyclohexane-1-carboxamide

A mixture of (1s,4s)-N-(3-methoxy-4-methylphenyl)-4-((4-methoxybenzyl)amino)-1-methylcyclohexane-1-carboxamide (0.38 g, 960 μmol) and palladium (10% on carbon, Type 487, dry, 0.10 g, 940 μmol) in methanol (10 mL) was charged into a septum-capped 20 ml vial with stir bar and purged with nitrogen for five minutes before placing in a steel bomb. The septum cap was pierced with a needle and the steel bomb sealed. The sealed bomb was pressurized with hydrogen gas to 60 psig and left to stir under pressure at ambient temperature overnight. After 20 hours LCMS indicated no starting material remained and a single peak with mass corresponding to expected product predominated. The reaction mixture was purged with nitrogen and then filtered through a pad of celite. The filtrate was concentrated under reduced pressure to afford (1r,4r)-4-amino-N-(3-methoxy-4-methylphenyl)-1-methylcyclohexane-1-carboxamide as a colorless solid (0.180 g, 650 μmol) LC/MS ESI 277.3 [M+H]⁺

### Step 2

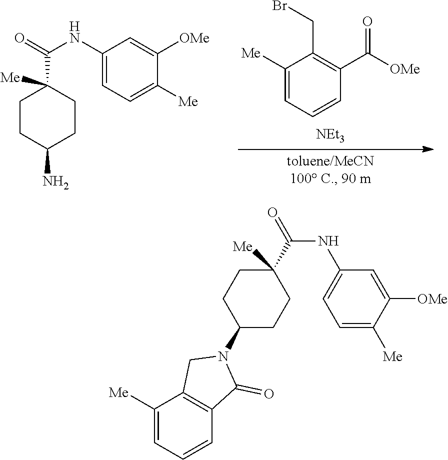 [SMILES: COc1cc(NC(=O)[C@]2(C)CC[C@@H](N)CC2)ccc1C ; COC(=O)c1cccc(C)c1CBr ; COc1cc(NC(=O)[C@]2(C)CC[C@@H](N3Cc4c(C)cccc4C3=O)CC2)ccc1C]

### (1r,4r)-N-(3-methoxy-4-methylphenyl)-1-methyl-4-(4-methyl-1-oxoisoindolin-2-yl)cyclohexane-1-carboxamide

A glass vial charged with methyl 2-(bromomethyl)-3-methylbenzoate (100 mg, 411 mmol), (1r,4r)-4-amino-N-(3-methoxy-4-methylphenyl)-1-methylcyclohexane-1-carboxamide (125 mg, 452 mmol) and triethylamine (50 mg, 490 mmol) in 3 mL toluene and 1 mL acetonitrile was sealed and heated to 100° C. for ninety minutes, after which time LCMS indicated no ester starting material remained and a peak with mass corresponding to desired product predominated. The reaction was equilibrated to room temperature and partitioned between 25 mL water and 25 mL ethyl acetate. The aqueous separation was extracted again with 25 mL ethyl acetate. The combined organic layers were washed with 25 mL brine, stirred over anhydrous magnesium sulfate, filtered and the filtrate concentrated under reduced pressure to afford crude product as a pink foam. The product was isolated by chromatography on ISCO CombiFlash Companion with a 10 gram Biotage Sfar silica gel column eluting with 25% ethyl acetate in hexane. Fractions containing product were combined and concentrated under reduced pressure to afford (1r,4r)-N-(3-methoxy-4-methylphenyl)-1-methyl-4-(4-methyl-1-oxoisoindolin-2-yl)cyclohexane-1-carboxamide as a colorless foamy solid (120 mg, 295 mmol). LC/MS ESI 407.2 [M+H]⁺. ¹H NMR (400 MHz, CHLOROFORM-d) δ ppm 1.24 (s, 3H) 1.40-1.55 (m, 2H) 1.60-1.84 (m, 4H) 2.11 (s, 3H) 2.23 (s, 3H) 2.28 (br d, J=12.30 Hz, 2H) 3.79 (s, 3H) 4.11 (s, 2H) 4.26 (tt, J=11.80, 4.14 Hz, 1H) 6.78 (dd, J=8.03, 2.01 Hz, 1H) 7.00 (d, J=8.28 Hz, 1H) 7.19-7.25 (m, 1H) 7.25-7.32 (m, 1H) 7.37 (d, J=2.01 Hz, 1H) 7.44 (s, 1H) 7.59 (d, J=7.53 Hz, 1H).

### Results

TABLE 3
Example Compounds

| Ex | Structure |
|---|---|
| 1 | 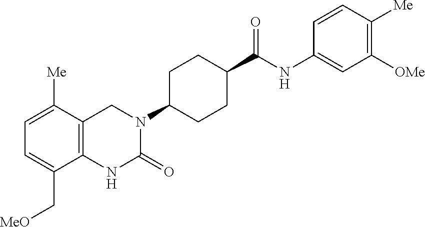 [SMILES: COCc1ccc(C)c2c1NC(=O)N([C@H]1CC[C@@H](C(=O)Nc3ccc(C)c(OC)c3)CC1)C2] |
| 2 | 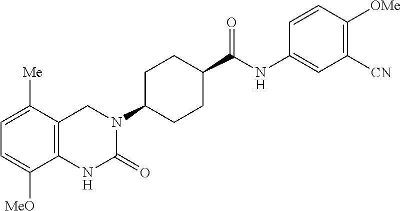 [SMILES: COc1ccc(NC(=O)[C@H]2CC[C@@H](N3Cc4c(C)ccc(OC)c4NC3=O)CC2)cc1C#N] |
| 3 | 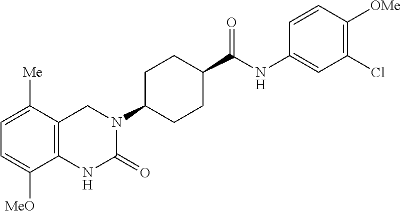 [SMILES: COc1ccc(NC(=O)[C@H]2CC[C@@H](N3Cc4c(C)ccc(OC)c4NC3=O)CC2)cc1Cl] |
| 4 | 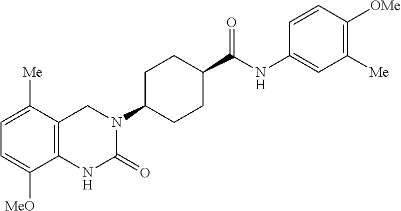 [SMILES: COc1ccc(NC(=O)[C@H]2CC[C@@H](N3Cc4c(C)ccc(OC)c4NC3=O)CC2)cc1C] |
| 5 | 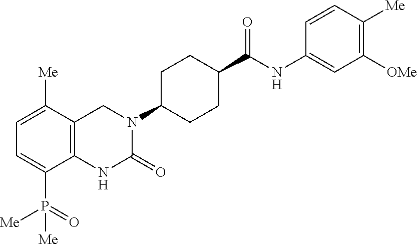 [SMILES: COc1cc(NC(=O)[C@H]2CC[C@@H](N3Cc4c(C)ccc(P(C)(C)=O)c4NC3=O)CC2)ccc1C] |
| 6 | 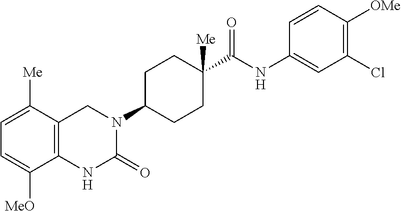 [SMILES: COc1ccc(NC(=O)[C@]2(C)CC[C@@H](N3Cc4c(C)ccc(OC)c4NC3=O)CC2)cc1Cl] |
| 7 | 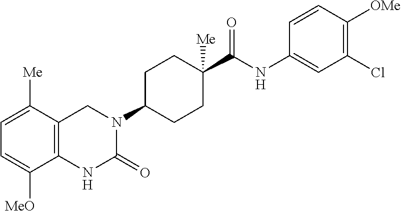 [SMILES: COc1ccc(NC(=O)[C@]2(C)CC[C@H](N3Cc4c(C)ccc(OC)c4NC3=O)CC2)cc1Cl] |
| 8 | 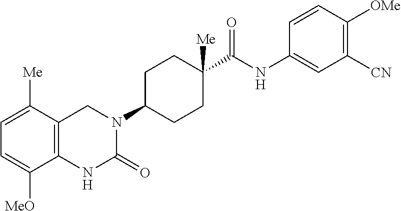 [SMILES: COc1ccc(NC(=O)[C@]2(C)CC[C@@H](N3Cc4c(C)ccc(OC)c4NC3=O)CC2)cc1C#N] |
| 9 | 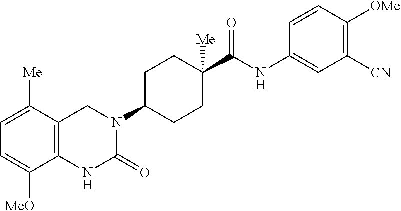 [SMILES: COc1ccc(NC(=O)[C@]2(C)CC[C@H](N3Cc4c(C)ccc(OC)c4NC3=O)CC2)cc1C#N] |
| 10 | 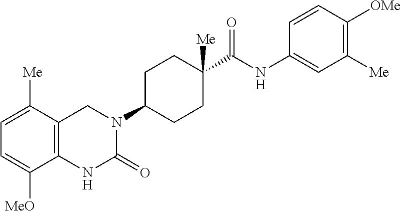 [SMILES: COc1ccc(NC(=O)[C@]2(C)CC[C@@H](N3Cc4c(C)ccc(OC)c4NC3=O)CC2)cc1C] |
| 11 | 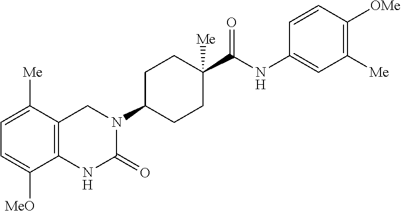 [SMILES: COc1ccc(NC(=O)[C@]2(C)CC[C@H](N3Cc4c(C)ccc(OC)c4NC3=O)CC2)cc1C] |
| 12 | 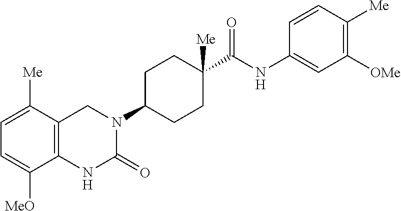 [SMILES: COc1cc(NC(=O)[C@]2(C)CC[C@@H](N3Cc4c(C)ccc(OC)c4NC3=O)CC2)ccc1C] |
| 13 | 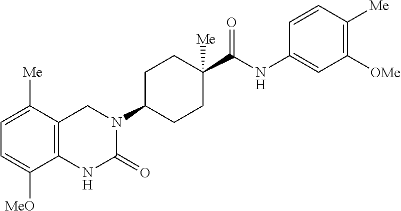 [SMILES: COc1cc(NC(=O)[C@]2(C)CC[C@H](N3Cc4c(C)ccc(OC)c4NC3=O)CC2)ccc1C] |
| 14 | 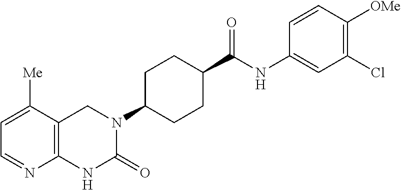 [SMILES: COc1ccc(NC(=O)[C@H]2CC[C@@H](N3Cc4c(C)ccnc4NC3=O)CC2)cc1Cl] |
| 15 | 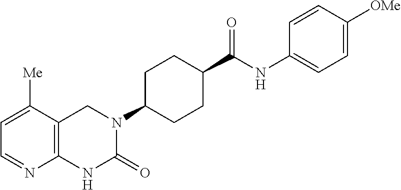 [SMILES: COc1ccc(NC(=O)[C@H]2CC[C@@H](N3Cc4c(C)ccnc4NC3=O)CC2)cc1] |
| 16 | 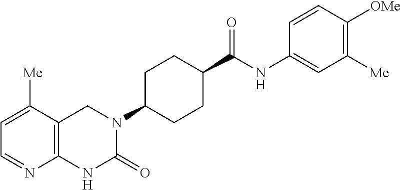 [SMILES: COc1ccc(NC(=O)[C@H]2CC[C@@H](N3Cc4c(C)ccnc4NC3=O)CC2)cc1C] |
| 17 | 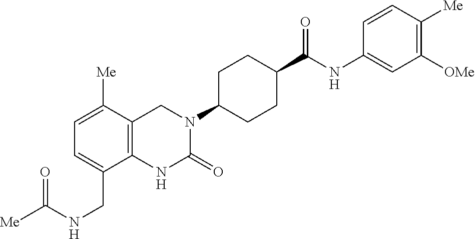 [SMILES: COc1cc(NC(=O)[C@H]2CC[C@@H](N3Cc4c(C)ccc(CNC(C)=O)c4NC3=O)CC2)ccc1C] |
| 18 | 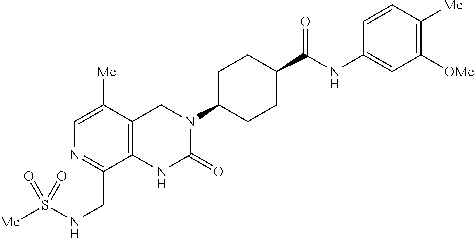 [SMILES: COc1cc(NC(=O)[C@H]2CC[C@@H](N3Cc4c(C)cnc(CNS(C)(=O)=O)c4NC3=O)CC2)ccc1C] |
| 19 | 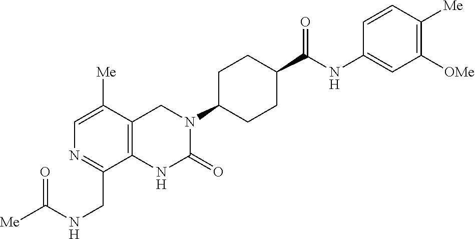 [SMILES: COc1cc(NC(=O)[C@H]2CC[C@@H](N3Cc4c(C)cnc(CNC(C)=O)c4NC3=O)CC2)ccc1C] |
| 20 | 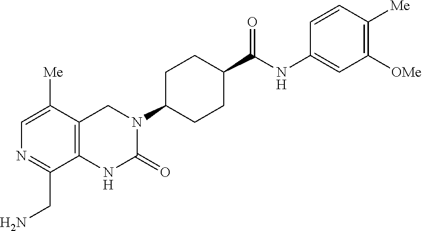 [SMILES: COc1cc(NC(=O)[C@H]2CC[C@@H](N3Cc4c(C)cnc(CN)c4NC3=O)CC2)ccc1C] |
| 21 | 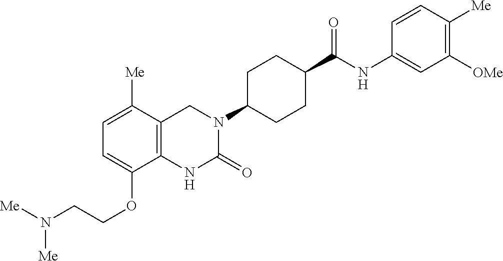 [SMILES: COc1cc(NC(=O)[C@H]2CC[C@@H](N3Cc4c(C)ccc(OCCN(C)C)c4NC3=O)CC2)ccc1C] |
| 22 | 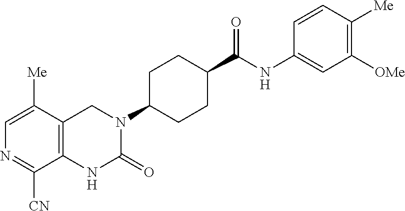 [SMILES: COc1cc(NC(=O)[C@H]2CC[C@@H](N3Cc4c(C)cnc(C#N)c4NC3=O)CC2)ccc1C] |
| 23 | 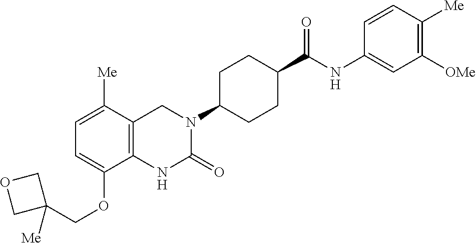 [SMILES: COc1cc(NC(=O)[C@H]2CC[C@@H](N3Cc4c(C)ccc(OCC5(C)COC5)c4NC3=O)CC2)ccc1C] |
| 24 | 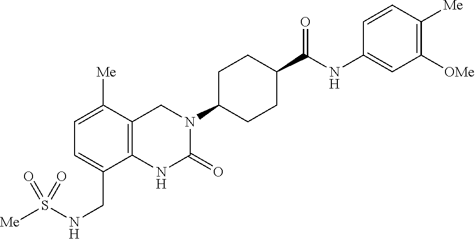 [SMILES: COc1cc(NC(=O)[C@H]2CC[C@@H](N3Cc4c(C)ccc(CNS(C)(=O)=O)c4NC3=O)CC2)ccc1C] |
| 25 | 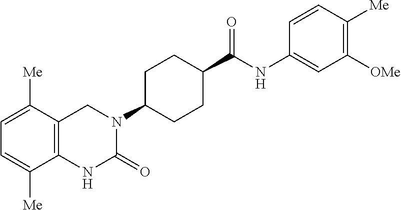 [SMILES: COc1cc(NC(=O)[C@H]2CC[C@@H](N3Cc4c(C)ccc(C)c4NC3=O)CC2)ccc1C] |
| 26 | 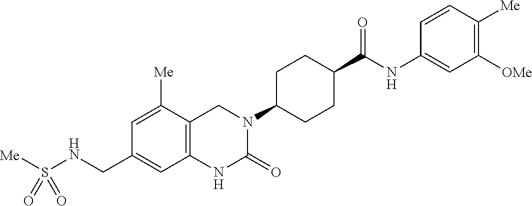 [SMILES: COc1cc(NC(=O)[C@H]2CC[C@@H](N3Cc4c(C)cc(CNS(C)(=O)=O)cc4NC3=O)CC2)ccc1C] |
| 27 | 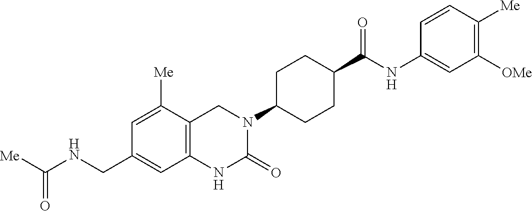 [SMILES: COc1cc(NC(=O)[C@H]2CC[C@@H](N3Cc4c(C)cc(CNC(C)=O)cc4NC3=O)CC2)ccc1C] |
| 28 | 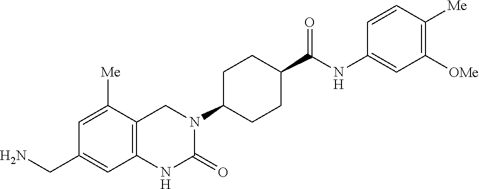 [SMILES: COc1cc(NC(=O)[C@H]2CC[C@@H](N3Cc4c(C)cc(CN)cc4NC3=O)CC2)ccc1C] |
| 29 | 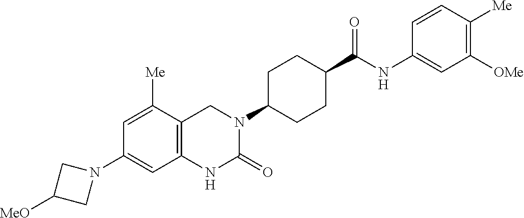 [SMILES: COc1cc(NC(=O)[C@H]2CC[C@@H](N3Cc4c(C)cc(N5CC(OC)C5)cc4NC3=O)CC2)ccc1C] |
| 30 | 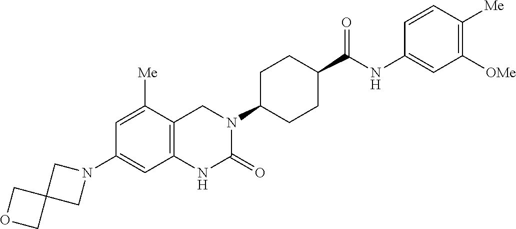 [SMILES: COc1cc(NC(=O)[C@H]2CC[C@@H](N3Cc4c(C)cc(N5CC6(COC6)C5)cc4NC3=O)CC2)ccc1C] |
| 31 | 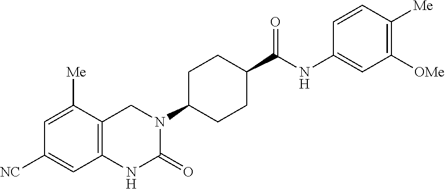 [SMILES: COc1cc(NC(=O)[C@H]2CC[C@@H](N3Cc4c(C)cc(C#N)cc4NC3=O)CC2)ccc1C] |
| 32 | 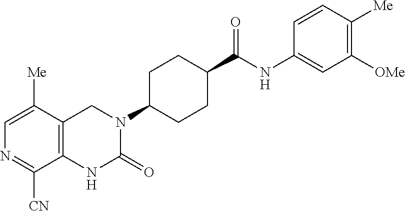 [SMILES: COc1cc(NC(=O)[C@H]2CC[C@@H](N3Cc4c(C)cnc(C#N)c4NC3=O)CC2)ccc1C] |
| 33 | 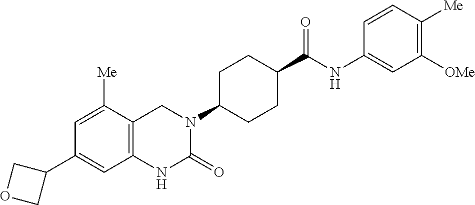 [SMILES: COc1cc(NC(=O)[C@H]2CC[C@@H](N3Cc4c(C)cc(C5COC5)cc4NC3=O)CC2)ccc1C] |
| 34 | 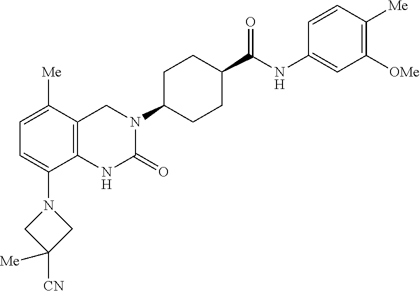 [SMILES: COc1cc(NC(=O)[C@H]2CC[C@@H](N3Cc4c(C)ccc(N5CC(C)(C#N)C5)c4NC3=O)CC2)ccc1C] |
| 35 | 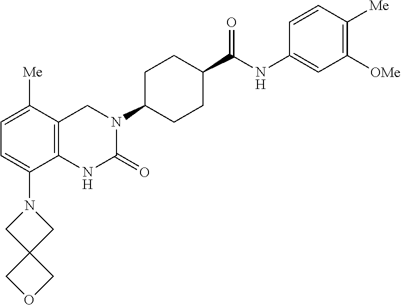 [SMILES: COc1cc(NC(=O)[C@H]2CC[C@@H](N3Cc4c(C)ccc(N5CC6(COC6)C5)c4NC3=O)CC2)ccc1C] |
| 36 | 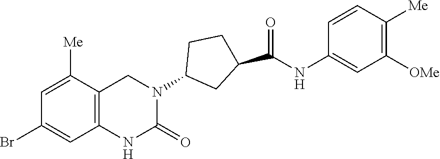 [SMILES: COc1cc(NC(=O)[C@@H]2CC[C@@H](N3Cc4c(C)cc(Br)cc4NC3=O)C2)ccc1C] |
| 37 | 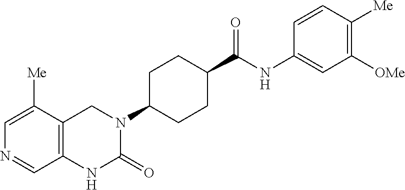 [SMILES: COc1cc(NC(=O)[C@H]2CC[C@@H](N3Cc4c(C)cncc4NC3=O)CC2)ccc1C] |
| 38 | 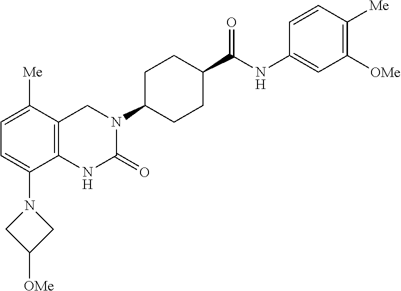 [SMILES: COc1cc(NC(=O)[C@H]2CC[C@@H](N3Cc4c(C)ccc(N5CC(OC)C5)c4NC3=O)CC2)ccc1C] |
| 39 | 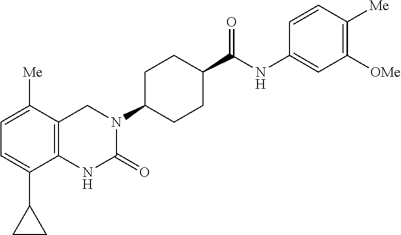 [SMILES: COc1cc(NC(=O)[C@H]2CC[C@@H](N3Cc4c(C)ccc(C5CC5)c4NC3=O)CC2)ccc1C] |
| 40 | 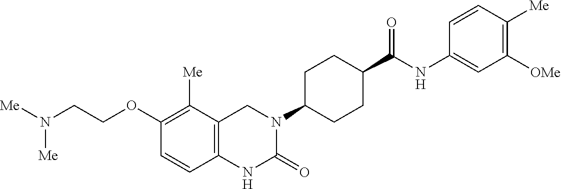 [SMILES: COc1cc(NC(=O)[C@H]2CC[C@@H](N3Cc4c(ccc(OCCN(C)C)c4C)NC3=O)CC2)ccc1C] |
| 41 | 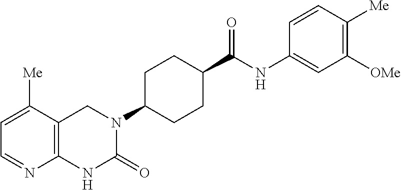 [SMILES: COc1cc(NC(=O)[C@H]2CC[C@@H](N3Cc4c(C)ccnc4NC3=O)CC2)ccc1C] |
| 42 | 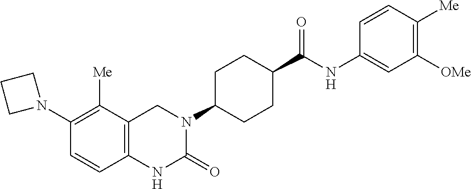 [SMILES: COc1cc(NC(=O)[C@H]2CC[C@@H](N3Cc4c(ccc(N5CCC5)c4C)NC3=O)CC2)ccc1C] |
| 43 | 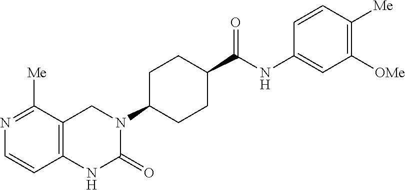 [SMILES: COc1cc(NC(=O)[C@H]2CC[C@@H](N3Cc4c(ccnc4C)NC3=O)CC2)ccc1C] |
| 44 | 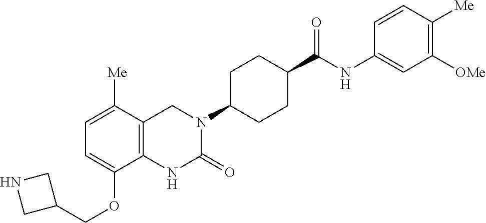 [SMILES: COc1cc(NC(=O)[C@H]2CC[C@@H](N3Cc4c(C)ccc(OCC5CNC5)c4NC3=O)CC2)ccc1C] |
| 45 | 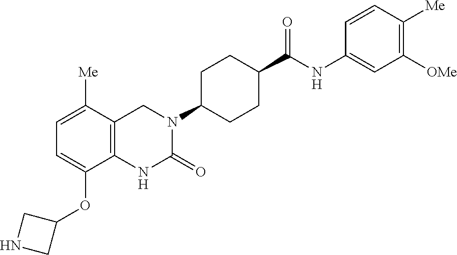 [SMILES: COc1cc(NC(=O)[C@H]2CC[C@@H](N3Cc4c(C)ccc(OC5CNC5)c4NC3=O)CC2)ccc1C] |
| 46 | 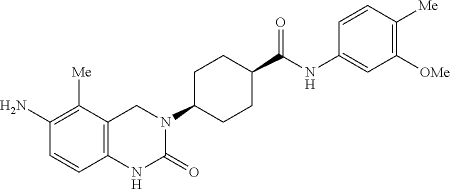 [SMILES: COc1cc(NC(=O)[C@H]2CC[C@@H](N3Cc4c(ccc(N)c4C)NC3=O)CC2)ccc1C] |
| 47 | 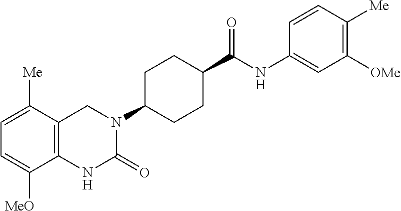 [SMILES: COc1cc(NC(=O)[C@H]2CC[C@@H](N3Cc4c(C)ccc(OC)c4NC3=O)CC2)ccc1C] |
| 48 | 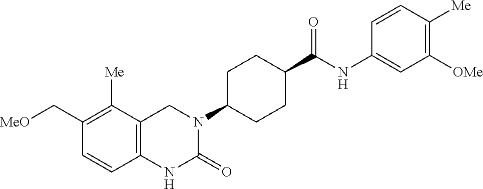 [SMILES: COCc1ccc2c(c1C)CN([C@H]1CC[C@@H](C(=O)Nc3ccc(C)c(OC)c3)CC1)C(=O)N2] |
| 49 | 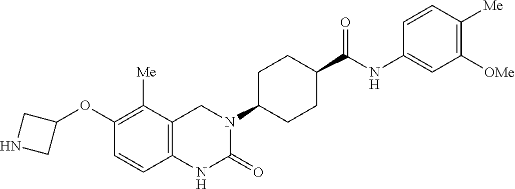 [SMILES: COc1cc(NC(=O)[C@H]2CC[C@@H](N3Cc4c(ccc(OC5CNC5)c4C)NC3=O)CC2)ccc1C] |
| 50 | 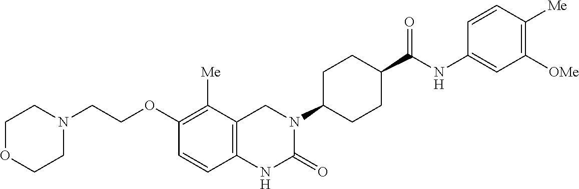 [SMILES: COc1cc(NC(=O)[C@H]2CC[C@@H](N3Cc4c(ccc(OCCN5CCOCC5)c4C)NC3=O)CC2)ccc1C] |
| 51 | 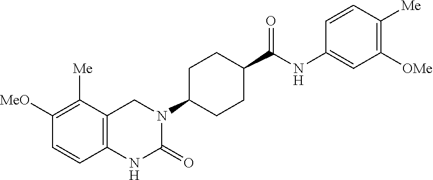 [SMILES: COc1cc(NC(=O)[C@H]2CC[C@@H](N3Cc4c(ccc(OC)c4C)NC3=O)CC2)ccc1C] |
| 52 | 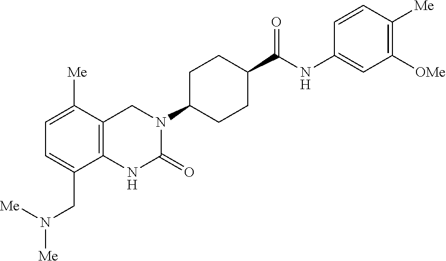 [SMILES: COc1cc(NC(=O)[C@H]2CC[C@@H](N3Cc4c(C)ccc(CN(C)C)c4NC3=O)CC2)ccc1C] |
| 53 | 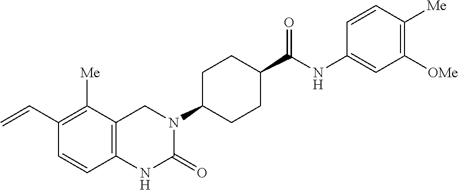 [SMILES: C=Cc1ccc2c(c1C)CN([C@H]1CC[C@@H](C(=O)Nc3ccc(C)c(OC)c3)CC1)C(=O)N2] |
| 54 | 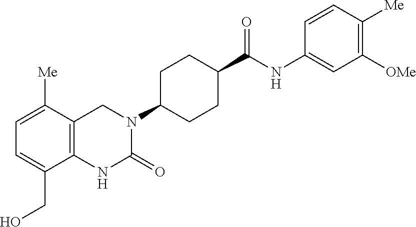 [SMILES: COc1cc(NC(=O)[C@H]2CC[C@@H](N3Cc4c(C)ccc(CO)c4NC3=O)CC2)ccc1C] |
| 55 | 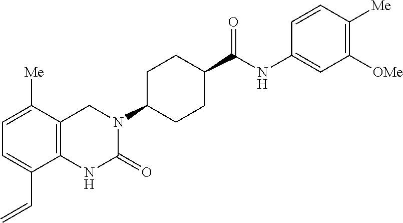 [SMILES: C=Cc1ccc(C)c2c1NC(=O)N([C@H]1CC[C@@H](C(=O)Nc3ccc(C)c(OC)c3)CC1)C2] |
| 56 | 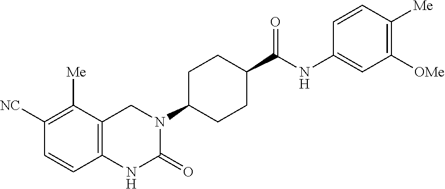 [SMILES: COc1cc(NC(=O)[C@H]2CC[C@@H](N3Cc4c(ccc(C#N)c4C)NC3=O)CC2)ccc1C] |
| 57 | 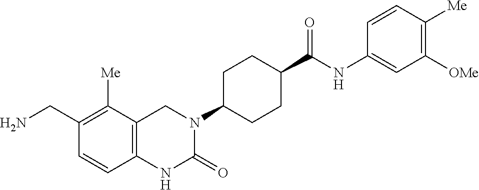 [SMILES: COc1cc(NC(=O)[C@H]2CC[C@@H](N3Cc4c(ccc(CN)c4C)NC3=O)CC2)ccc1C] |
| 58 | 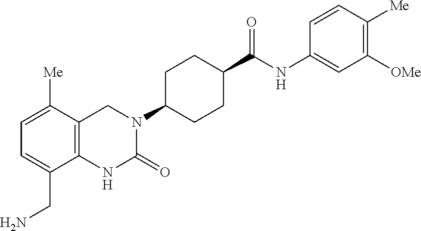 [SMILES: COc1cc(NC(=O)[C@H]2CC[C@@H](N3Cc4c(C)ccc(CN)c4NC3=O)CC2)ccc1C] |
| 59 | 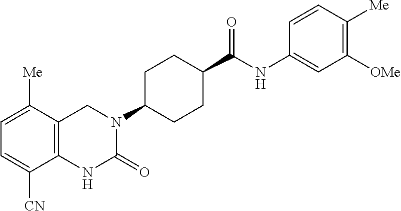 [SMILES: COc1cc(NC(=O)[C@H]2CC[C@@H](N3Cc4c(C)ccc(C#N)c4NC3=O)CC2)ccc1C] |
| 60 | 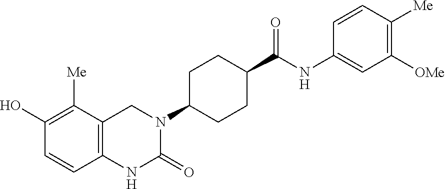 [SMILES: COc1cc(NC(=O)[C@H]2CC[C@@H](N3Cc4c(ccc(O)c4C)NC3=O)CC2)ccc1C] |
| 61 | 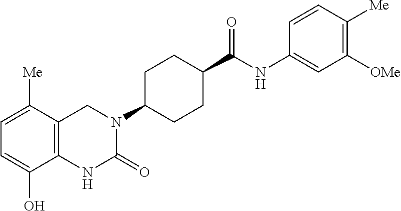 [SMILES: COc1cc(NC(=O)[C@H]2CC[C@@H](N3Cc4c(C)ccc(O)c4NC3=O)CC2)ccc1C] |
| 62 | 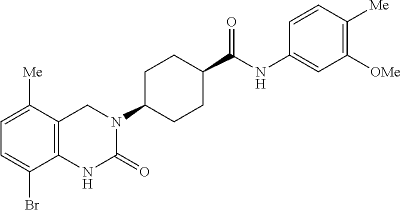 [SMILES: COc1cc(NC(=O)[C@H]2CC[C@@H](N3Cc4c(C)ccc(Br)c4NC3=O)CC2)ccc1C] |
| 63 | 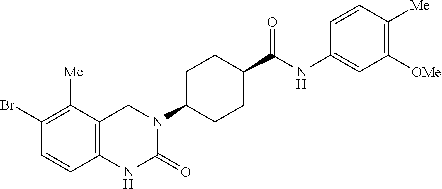 [SMILES: COc1cc(NC(=O)[C@H]2CC[C@@H](N3Cc4c(ccc(Br)c4C)NC3=O)CC2)ccc1C] |
| 64 | 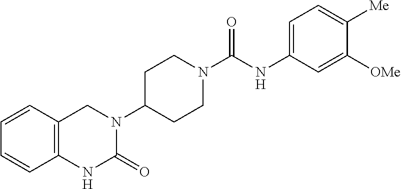 [SMILES: COc1cc(NC(=O)N2CCC(N3Cc4ccccc4NC3=O)CC2)ccc1C] |
| 65 | 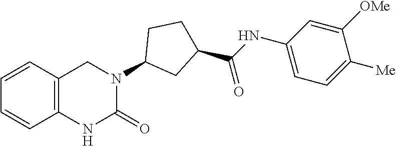 [SMILES: COc1cc(NC(=O)[C@@H]2CC[C@H](N3Cc4ccccc4NC3=O)C2)ccc1C] |
| 66 | 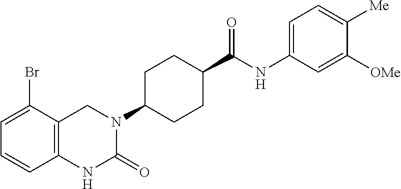 [SMILES: COc1cc(NC(=O)[C@H]2CC[C@@H](N3Cc4c(Br)cccc4NC3=O)CC2)ccc1C] |
| 67 | 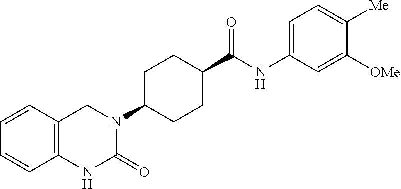 [SMILES: COc1cc(NC(=O)[C@H]2CC[C@@H](N3Cc4ccccc4NC3=O)CC2)ccc1C] |
| 68 | 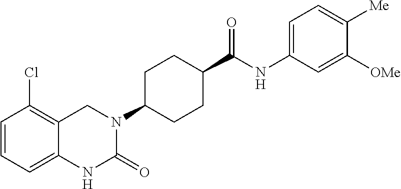 [SMILES: COc1cc(NC(=O)[C@H]2CC[C@@H](N3Cc4c(Cl)cccc4NC3=O)CC2)ccc1C] |
| 69 | 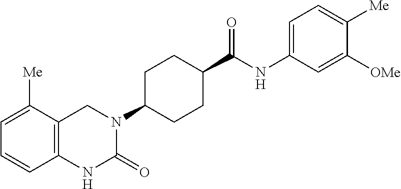 [SMILES: COc1cc(NC(=O)[C@H]2CC[C@@H](N3Cc4c(C)cccc4NC3=O)CC2)ccc1C] |
| 70 | 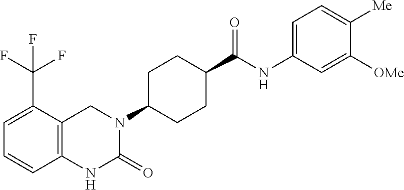 [SMILES: COc1cc(NC(=O)[C@H]2CC[C@@H](N3Cc4c(cccc4C(F)(F)F)NC3=O)CC2)ccc1C] |
| 71 | 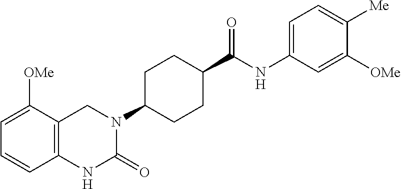 [SMILES: COc1cc(NC(=O)[C@H]2CC[C@@H](N3Cc4c(cccc4OC)NC3=O)CC2)ccc1C] |
| 72 | 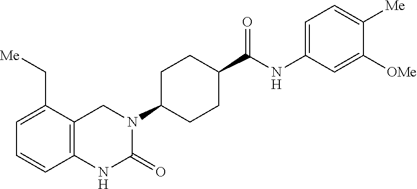 [SMILES: CCc1cccc2c1CN([C@H]1CC[C@@H](C(=O)Nc3ccc(C)c(OC)c3)CC1)C(=O)N2] |
| 73 | 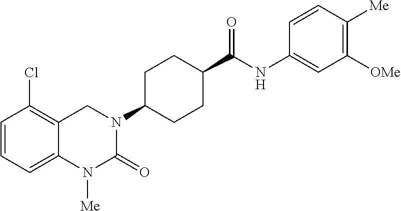 [SMILES: COc1cc(NC(=O)[C@H]2CC[C@@H](N3Cc4c(Cl)cccc4N(C)C3=O)CC2)ccc1C] |
| 74 | 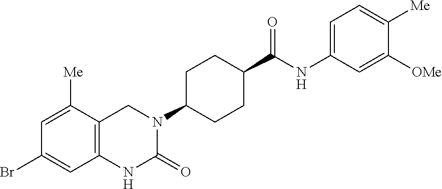 [SMILES: COc1cc(NC(=O)[C@H]2CC[C@@H](N3Cc4c(C)cc(Br)cc4NC3=O)CC2)ccc1C] |
| 75 | 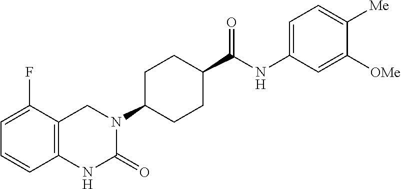 [SMILES: COc1cc(NC(=O)[C@H]2CC[C@@H](N3Cc4c(F)cccc4NC3=O)CC2)ccc1C] |
| 76 | 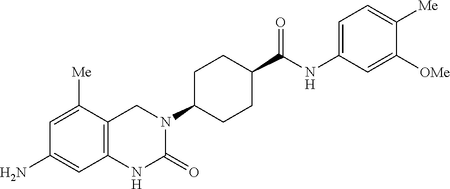 [SMILES: COc1cc(NC(=O)[C@H]2CC[C@@H](N3Cc4c(C)cc(N)cc4NC3=O)CC2)ccc1C] |
| 77 | 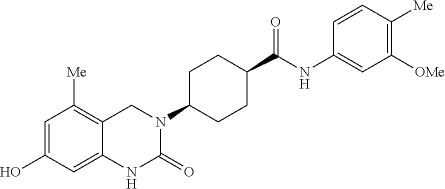 [SMILES: COc1cc(NC(=O)[C@H]2CC[C@@H](N3Cc4c(C)cc(O)cc4NC3=O)CC2)ccc1C] |
| 78 | 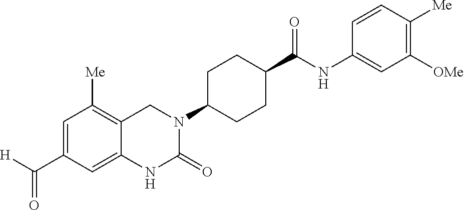 [SMILES: COc1cc(NC(=O)[C@H]2CC[C@@H](N3Cc4c(C)cc(C=O)cc4NC3=O)CC2)ccc1C] |
| 79 | 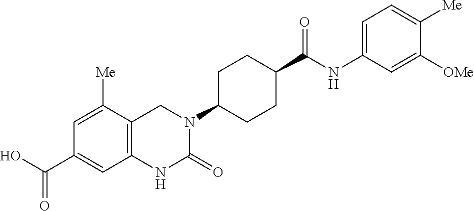 [SMILES: COc1cc(NC(=O)[C@H]2CC[C@@H](N3Cc4c(C)cc(C(=O)O)cc4NC3=O)CC2)ccc1C] |
| 80 | 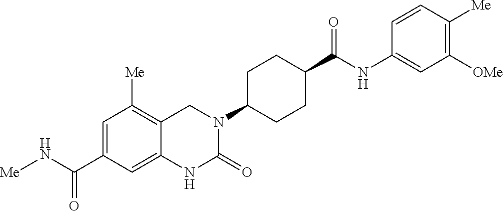 [SMILES: CNC(=O)c1cc(C)c2c(c1)NC(=O)N([C@H]1CC[C@@H](C(=O)Nc3ccc(C)c(OC)c3)CC1)C2] |
| 81 | 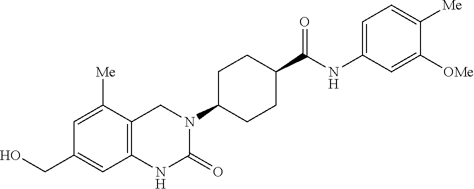 [SMILES: COc1cc(NC(=O)[C@H]2CC[C@@H](N3Cc4c(C)cc(CO)cc4NC3=O)CC2)ccc1C] |
| 82 | 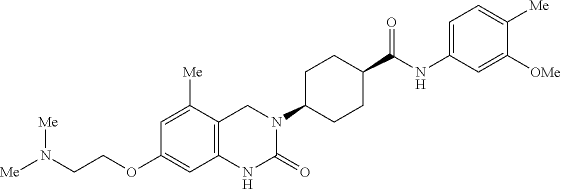 [SMILES: COc1cc(NC(=O)[C@H]2CC[C@@H](N3Cc4c(C)cc(OCCN(C)C)cc4NC3=O)CC2)ccc1C] |
| 83 | 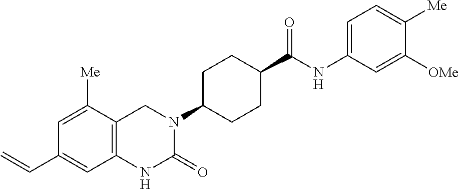 [SMILES: C=Cc1cc(C)c2c(c1)NC(=O)N([C@H]1CC[C@@H](C(=O)Nc3ccc(C)c(OC)c3)CC1)C2] |
| 84 | 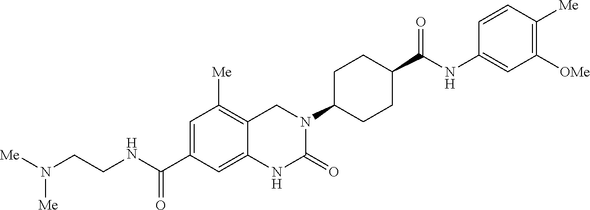 [SMILES: COc1cc(NC(=O)[C@H]2CC[C@@H](N3Cc4c(C)cc(C(=O)NCCN(C)C)cc4NC3=O)CC2)ccc1C] |
| 85 | 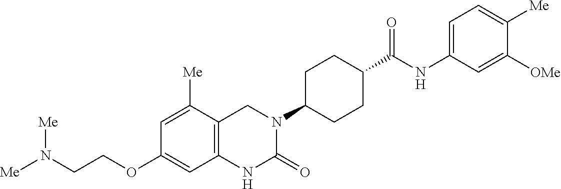 [SMILES: COc1cc(NC(=O)[C@H]2CC[C@H](N3Cc4c(C)cc(OCCN(C)C)cc4NC3=O)CC2)ccc1C] |
| 86 | 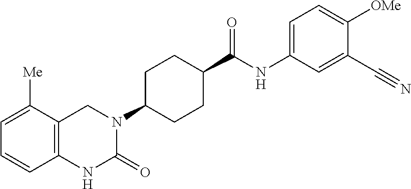 [SMILES: COc1ccc(NC(=O)[C@H]2CC[C@@H](N3Cc4c(C)cccc4NC3=O)CC2)cc1C#N] |
| 87 | 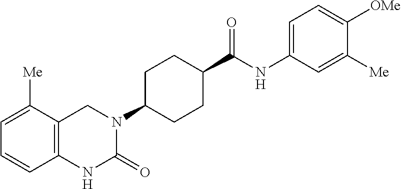 [SMILES: COc1ccc(NC(=O)[C@H]2CC[C@@H](N3Cc4c(C)cccc4NC3=O)CC2)cc1C] |
| 88 | 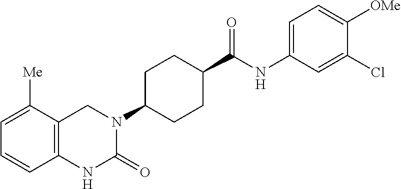 [SMILES: COc1ccc(NC(=O)[C@H]2CC[C@@H](N3Cc4c(C)cccc4NC3=O)CC2)cc1Cl] |
| 89 | 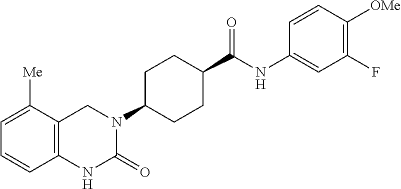 [SMILES: COc1ccc(NC(=O)[C@H]2CC[C@@H](N3Cc4c(C)cccc4NC3=O)CC2)cc1F] |
| 90 | 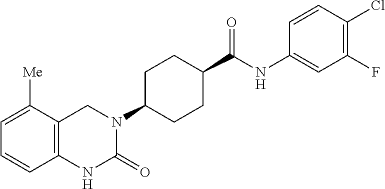 [SMILES: Cc1cccc2c1CN([C@H]1CC[C@@H](C(=O)Nc3ccc(Cl)c(F)c3)CC1)C(=O)N2] |
| 91 | 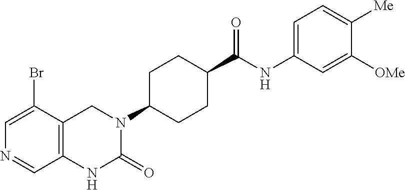 [SMILES: COc1cc(NC(=O)[C@H]2CC[C@@H](N3Cc4c(Br)cncc4NC3=O)CC2)ccc1C] |
| 92 | 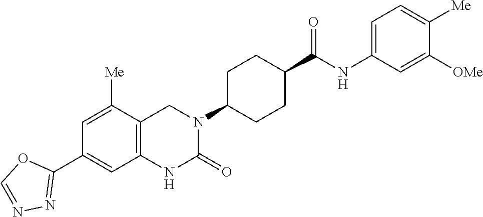 [SMILES: COc1cc(NC(=O)[C@H]2CC[C@@H](N3Cc4c(C)cc(-c5nnco5)cc4NC3=O)CC2)ccc1C] |
| 93 | 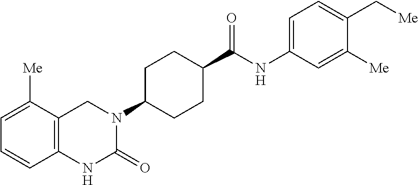 [SMILES: CCc1ccc(NC(=O)[C@H]2CC[C@@H](N3Cc4c(C)cccc4NC3=O)CC2)cc1C] |
| 94 | 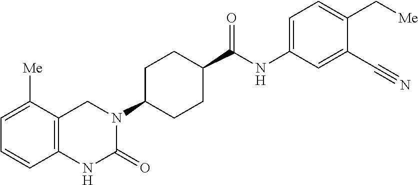 [SMILES: CCc1ccc(NC(=O)[C@H]2CC[C@@H](N3Cc4c(C)cccc4NC3=O)CC2)cc1C#N] |
| 95 | 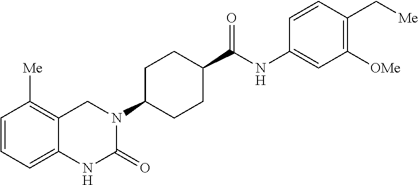 [SMILES: CCc1ccc(NC(=O)[C@H]2CC[C@@H](N3Cc4c(C)cccc4NC3=O)CC2)cc1OC] |
| 96 | 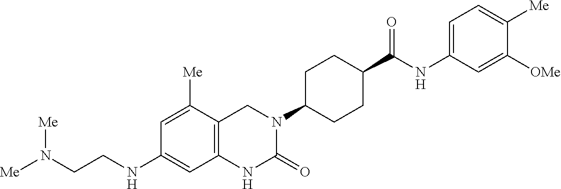 [SMILES: COc1cc(NC(=O)[C@H]2CC[C@@H](N3Cc4c(C)cc(NCCN(C)C)cc4NC3=O)CC2)ccc1C] |
| 97 | 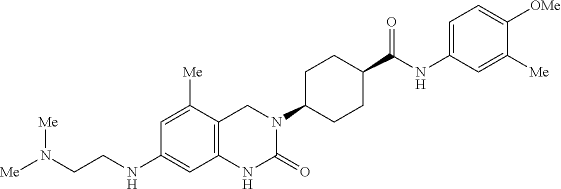 [SMILES: COc1ccc(NC(=O)[C@H]2CC[C@@H](N3Cc4c(C)cc(NCCN(C)C)cc4NC3=O)CC2)cc1C] |
| 98 | 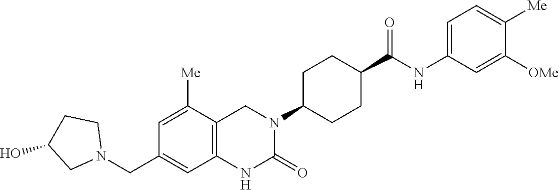 [SMILES: COc1cc(NC(=O)[C@H]2CC[C@@H](N3Cc4c(C)cc(CN5CC[C@@H](O)C5)cc4NC3=O)CC2)ccc1C] |
| 99 | 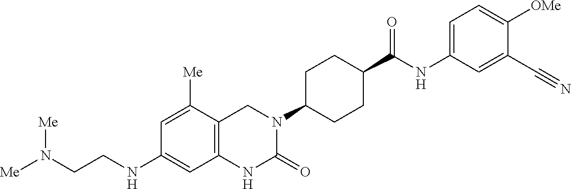 [SMILES: COc1ccc(NC(=O)[C@H]2CC[C@@H](N3Cc4c(C)cc(NCCN(C)C)cc4NC3=O)CC2)cc1C#N] |
| 100 | 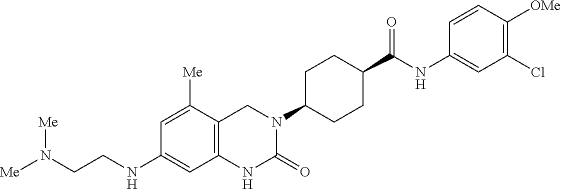 [SMILES: COc1ccc(NC(=O)[C@H]2CC[C@@H](N3Cc4c(C)cc(NCCN(C)C)cc4NC3=O)CC2)cc1Cl] |
| 101 | 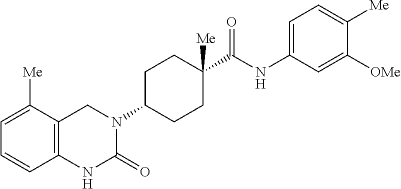 [SMILES: COc1cc(NC(=O)[C@]2(C)CC[C@H](N3Cc4c(C)cccc4NC3=O)CC2)ccc1C] |
| 102 | 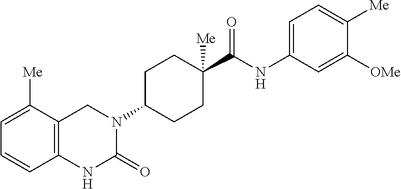 [SMILES: COc1cc(NC(=O)[C@]2(C)CC[C@@H](N3Cc4c(C)cccc4NC3=O)CC2)ccc1C] |
| 103 | 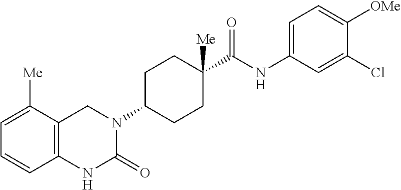 [SMILES: COc1ccc(NC(=O)[C@]2(C)CC[C@H](N3Cc4c(C)cccc4NC3=O)CC2)cc1Cl] |
| 104 | 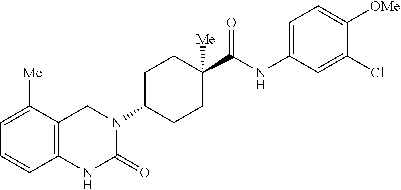 [SMILES: COc1ccc(NC(=O)[C@]2(C)CC[C@@H](N3Cc4c(C)cccc4NC3=O)CC2)cc1Cl] |
| 105 | 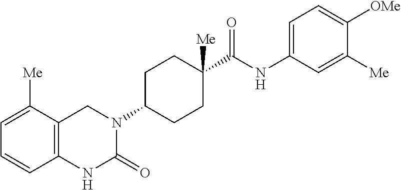 [SMILES: COc1ccc(NC(=O)[C@]2(C)CC[C@H](N3Cc4c(C)cccc4NC3=O)CC2)cc1C] |
| 106 | 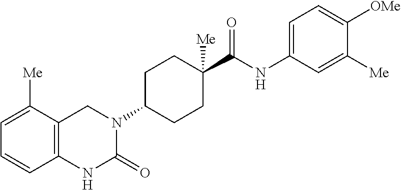 [SMILES: COc1ccc(NC(=O)[C@]2(C)CC[C@@H](N3Cc4c(C)cccc4NC3=O)CC2)cc1C] |
| 107 | 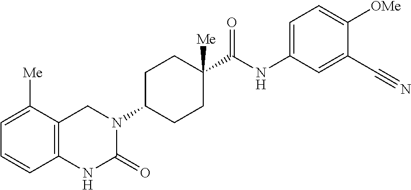 [SMILES: COc1ccc(NC(=O)[C@]2(C)CC[C@H](N3Cc4c(C)cccc4NC3=O)CC2)cc1C#N] |
| 108 | 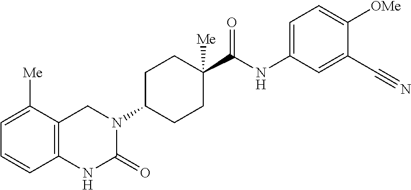 [SMILES: COc1ccc(NC(=O)[C@]2(C)CC[C@@H](N3Cc4c(C)cccc4NC3=O)CC2)cc1C#N] |
| 109 | 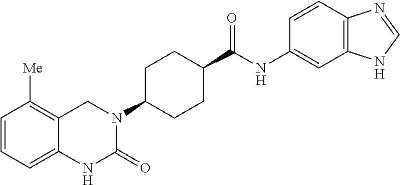 [SMILES: Cc1cccc2c1CN([C@H]1CC[C@@H](C(=O)Nc3ccc4nc[nH]c4c3)CC1)C(=O)N2] |
| 110 | 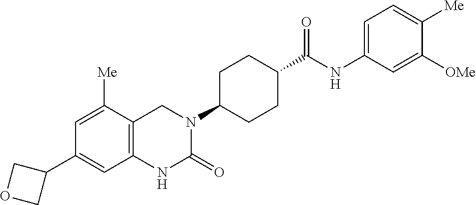 [SMILES: COc1cc(NC(=O)[C@H]2CC[C@H](N3Cc4c(C)cc(C5COC5)cc4NC3=O)CC2)ccc1C] |
| 111 | 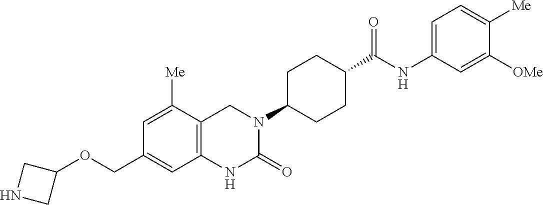 [SMILES: COc1cc(NC(=O)[C@H]2CC[C@H](N3Cc4c(C)cc(COC5CNC5)cc4NC3=O)CC2)ccc1C] |
| 112 | 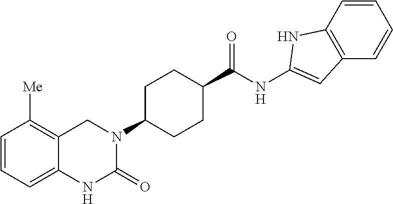 [SMILES: Cc1cccc2c1CN([C@H]1CC[C@@H](C(=O)Nc3cc4ccccc4[nH]3)CC1)C(=O)N2] |
| 113 | 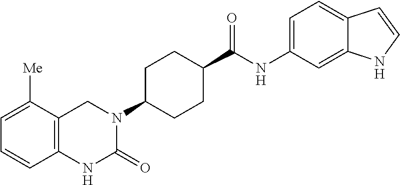 [SMILES: Cc1cccc2c1CN([C@H]1CC[C@@H](C(=O)Nc3ccc4cc[nH]c4c3)CC1)C(=O)N2] |
| 114 | 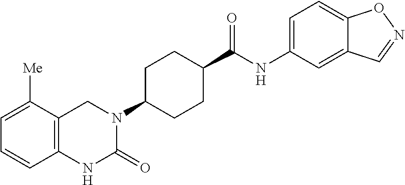 [SMILES: Cc1cccc2c1CN([C@H]1CC[C@@H](C(=O)Nc3ccc4oncc4c3)CC1)C(=O)N2] |
| 115 | 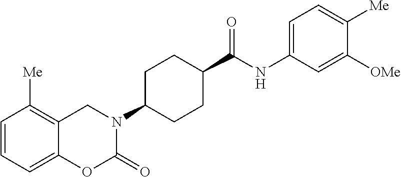 [SMILES: COc1cc(NC(=O)[C@H]2CC[C@@H](N3Cc4c(C)cccc4OC3=O)CC2)ccc1C] |
| 116 | 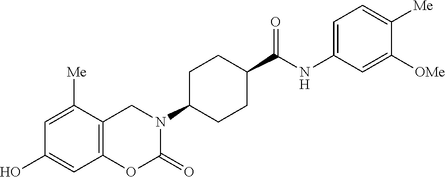 [SMILES: COc1cc(NC(=O)[C@H]2CC[C@@H](N3Cc4c(C)cc(O)cc4OC3=O)CC2)ccc1C] |
| 117 | 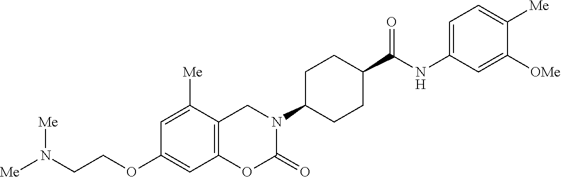 [SMILES: COc1cc(NC(=O)[C@H]2CC[C@@H](N3Cc4c(C)cc(OCCN(C)C)cc4OC3=O)CC2)ccc1C] |
| 118 | 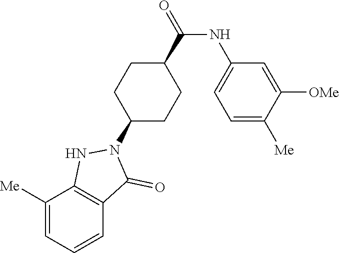 [SMILES: COc1cc(NC(=O)[C@H]2CC[C@@H](n3[nH]c4c(C)cccc4c3=O)CC2)ccc1C] |
| 119 | 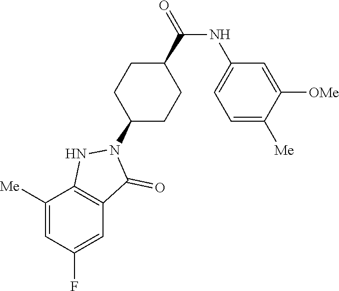 [SMILES: COc1cc(NC(=O)[C@H]2CC[C@@H](n3[nH]c4c(C)cc(F)cc4c3=O)CC2)ccc1C] |
| 120 | 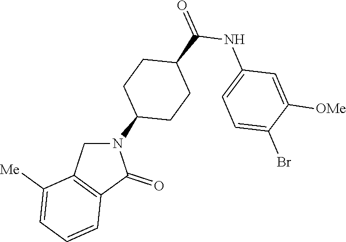 [SMILES: COc1cc(NC(=O)[C@H]2CC[C@@H](N3Cc4c(C)cccc4C3=O)CC2)ccc1Br] |
| 121 | 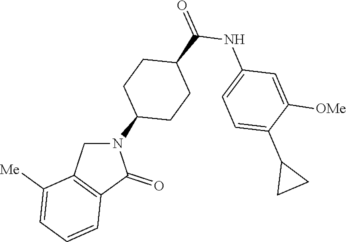 [SMILES: COc1cc(NC(=O)[C@H]2CC[C@@H](N3Cc4c(C)cccc4C3=O)CC2)ccc1C1CC1] |
| 122 | 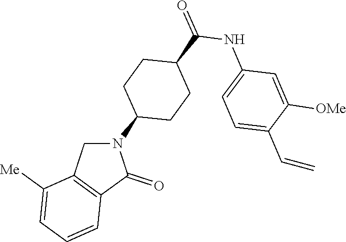 [SMILES: C=Cc1ccc(NC(=O)[C@H]2CC[C@@H](N3Cc4c(C)cccc4C3=O)CC2)cc1OC] |
| 123 | 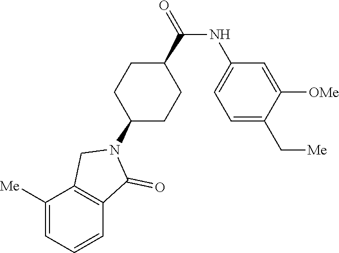 [SMILES: CCc1ccc(NC(=O)[C@H]2CC[C@@H](N3Cc4c(C)cccc4C3=O)CC2)cc1OC] |
| 124 | 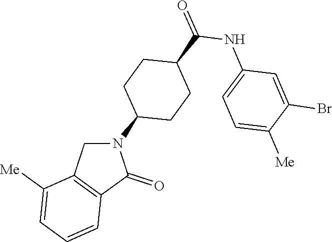 [SMILES: Cc1ccc(NC(=O)[C@H]2CC[C@@H](N3Cc4c(C)cccc4C3=O)CC2)cc1Br] |
| 125 | 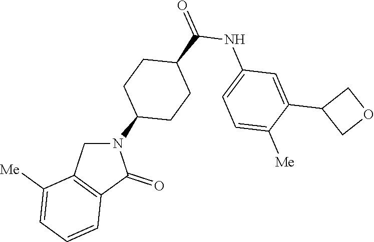 [SMILES: Cc1ccc(NC(=O)[C@H]2CC[C@@H](N3Cc4c(C)cccc4C3=O)CC2)cc1C1COC1] |
| 126 | 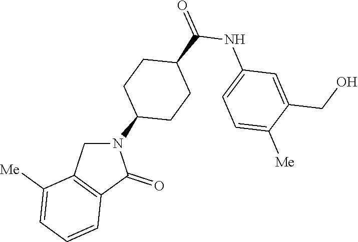 [SMILES: Cc1ccc(NC(=O)[C@H]2CC[C@@H](N3Cc4c(C)cccc4C3=O)CC2)cc1CO] |
| 127 | 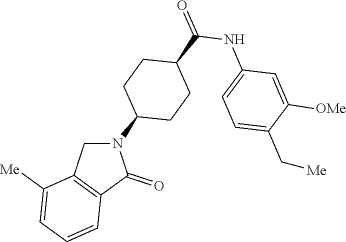 [SMILES: CCc1ccc(NC(=O)[C@H]2CC[C@@H](N3Cc4c(C)cccc4C3=O)CC2)cc1OC] |
| 128 | 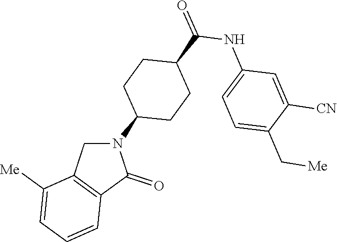 [SMILES: CCc1ccc(NC(=O)[C@H]2CC[C@@H](N3Cc4c(C)cccc4C3=O)CC2)cc1C#N] |
| 129 | 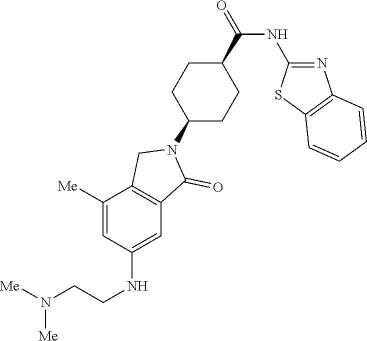 [SMILES: Cc1cc(NCCN(C)C)cc2c1CN([C@H]1CC[C@@H](C(=O)Nc3nc4ccccc4s3)CC1)C2=O] |
| 130 | 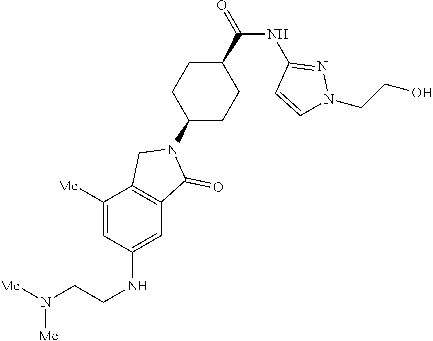 [SMILES: Cc1cc(NCCN(C)C)cc2c1CN([C@H]1CC[C@@H](C(=O)Nc3ccn(CCO)n3)CC1)C2=O] |
| 131 | 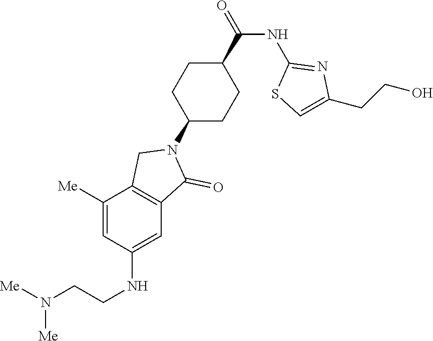 [SMILES: Cc1cc(NCCN(C)C)cc2c1CN([C@H]1CC[C@@H](C(=O)Nc3nc(CCO)cs3)CC1)C2=O] |
| 132 | 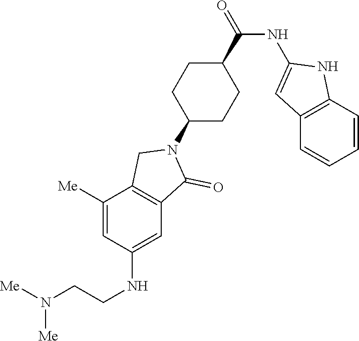 [SMILES: Cc1cc(NCCN(C)C)cc2c1CN([C@H]1CC[C@@H](C(=O)Nc3cc4ccccc4[nH]3)CC1)C2=O] |
| 133 | 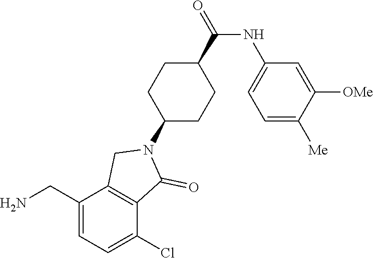 [SMILES: COc1cc(NC(=O)[C@H]2CC[C@@H](N3Cc4c(CN)ccc(Cl)c4C3=O)CC2)ccc1C] |
| 134 | 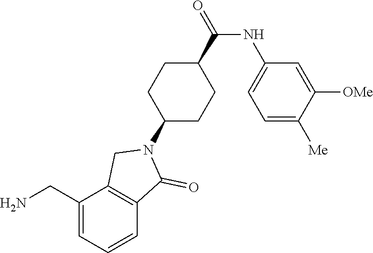 [SMILES: COc1cc(NC(=O)[C@H]2CC[C@@H](N3Cc4c(CN)cccc4C3=O)CC2)ccc1C] |
| 135 | 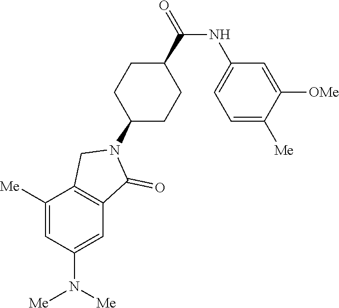 [SMILES: COc1cc(NC(=O)[C@H]2CC[C@@H](N3Cc4c(C)cc(N(C)C)cc4C3=O)CC2)ccc1C] |
| 136 | 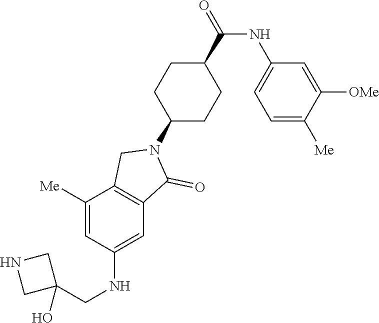 [SMILES: COc1cc(NC(=O)[C@H]2CC[C@@H](N3Cc4c(C)cc(NCC5(O)CNC5)cc4C3=O)CC2)ccc1C] |
| 137 | 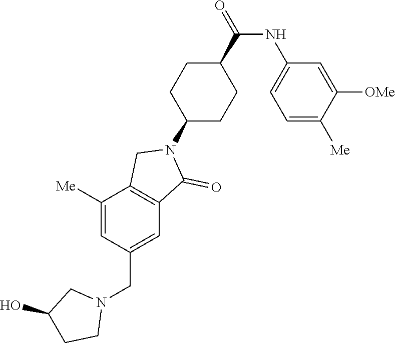 [SMILES: COc1cc(NC(=O)[C@H]2CC[C@@H](N3Cc4c(C)cc(CN5CC[C@@H](O)C5)cc4C3=O)CC2)ccc1C] |
| 138 | 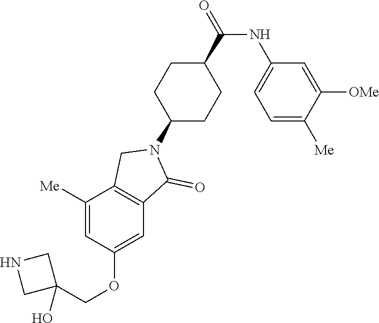 [SMILES: COc1cc(NC(=O)[C@H]2CC[C@@H](N3Cc4c(C)cc(OCC5(O)CNC5)cc4C3=O)CC2)ccc1C] |
| 139 | 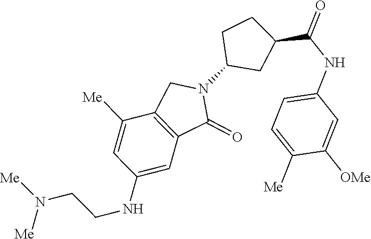 [SMILES: COc1cc(NC(=O)[C@@H]2CC[C@@H](N3Cc4c(C)cc(NCCN(C)C)cc4C3=O)C2)ccc1C] |
| 140 | 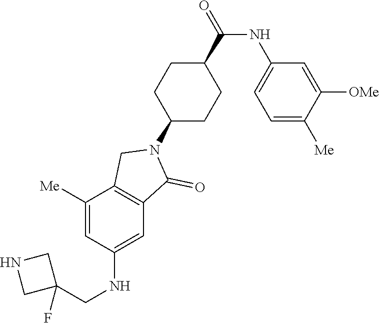 [SMILES: COc1cc(NC(=O)[C@H]2CC[C@@H](N3Cc4c(C)cc(NCC5(F)CNC5)cc4C3=O)CC2)ccc1C] |
| 141 | 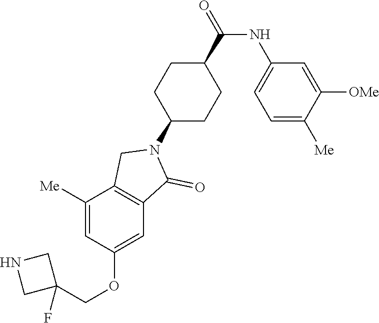 [SMILES: COc1cc(NC(=O)[C@H]2CC[C@@H](N3Cc4c(C)cc(OCC5(F)CNC5)cc4C3=O)CC2)ccc1C] |
| 142 | 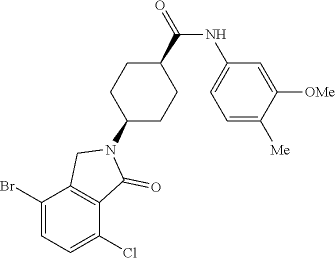 [SMILES: COc1cc(NC(=O)[C@H]2CC[C@@H](N3Cc4c(Br)ccc(Cl)c4C3=O)CC2)ccc1C] |
| 143 | 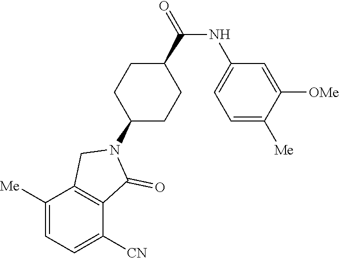 [SMILES: COc1cc(NC(=O)[C@H]2CC[C@@H](N3Cc4c(C)ccc(C#N)c4C3=O)CC2)ccc1C] |
| 144 | 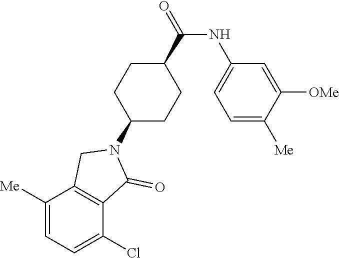 [SMILES: COc1cc(NC(=O)[C@H]2CC[C@@H](N3Cc4c(C)ccc(Cl)c4C3=O)CC2)ccc1C] |
| 145 | 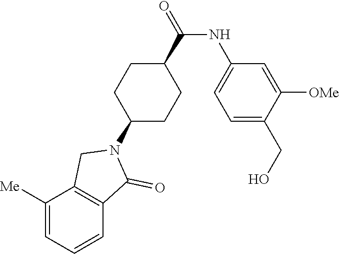 [SMILES: COc1cc(NC(=O)[C@H]2CC[C@@H](N3Cc4c(C)cccc4C3=O)CC2)ccc1CO] |
| 146 | 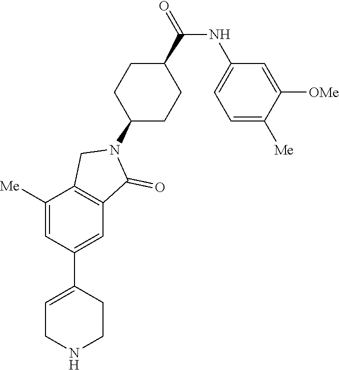 [SMILES: COc1cc(NC(=O)[C@H]2CC[C@@H](N3Cc4c(C)cc(C5=CCNCC5)cc4C3=O)CC2)ccc1C] |
| 147 | 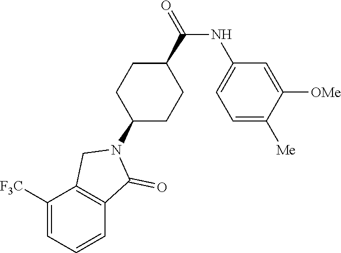 [SMILES: COc1cc(NC(=O)[C@H]2CC[C@@H](N3Cc4c(cccc4C(F)(F)F)C3=O)CC2)ccc1C] |
| 148 | 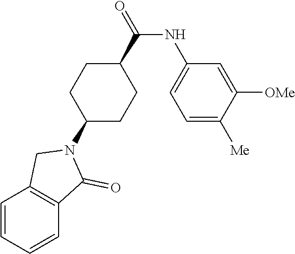 [SMILES: COc1cc(NC(=O)[C@H]2CC[C@@H](N3Cc4ccccc4C3=O)CC2)ccc1C] |
| 149 | 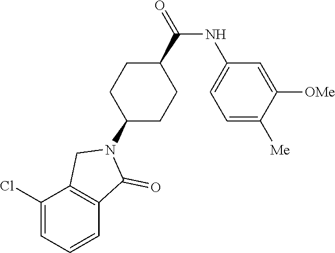 [SMILES: COc1cc(NC(=O)[C@H]2CC[C@@H](N3Cc4c(Cl)cccc4C3=O)CC2)ccc1C] |
| 150 | 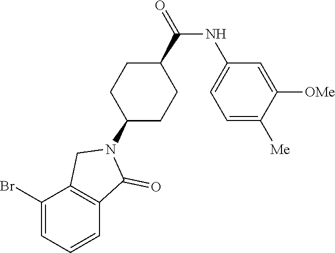 [SMILES: COc1cc(NC(=O)[C@H]2CC[C@@H](N3Cc4c(Br)cccc4C3=O)CC2)ccc1C] |
| 151 | 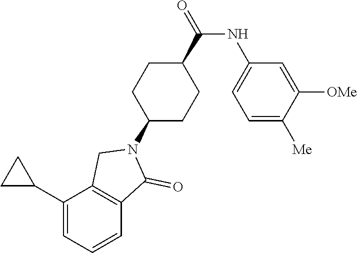 [SMILES: COc1cc(NC(=O)[C@H]2CC[C@@H](N3Cc4c(cccc4C4CC4)C3=O)CC2)ccc1C] |
| 152 | 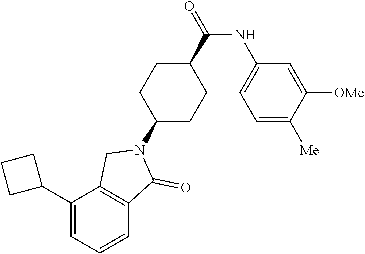 [SMILES: COc1cc(NC(=O)[C@H]2CC[C@@H](N3Cc4c(cccc4C4CCC4)C3=O)CC2)ccc1C] |
| 153 | 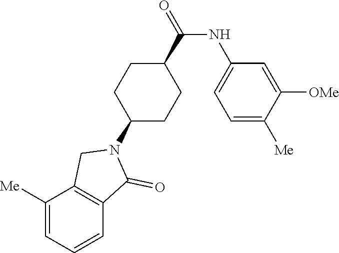 [SMILES: COc1cc(NC(=O)[C@H]2CC[C@@H](N3Cc4c(C)cccc4C3=O)CC2)ccc1C] |
| 154 | 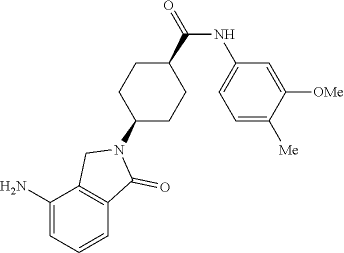 [SMILES: COc1cc(NC(=O)[C@H]2CC[C@@H](N3Cc4c(N)cccc4C3=O)CC2)ccc1C] |
| 155 | 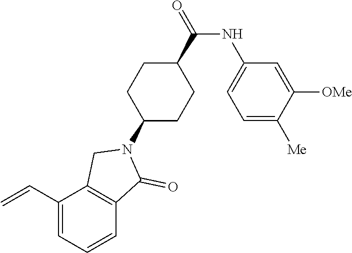 [SMILES: C=Cc1cccc2c1CN([C@H]1CC[C@@H](C(=O)Nc3ccc(C)c(OC)c3)CC1)C2=O] |
| 156 | 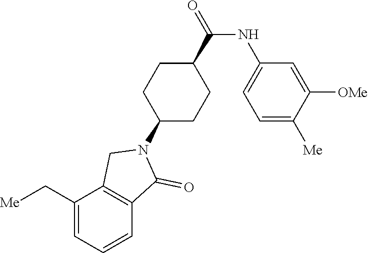 [SMILES: CCc1cccc2c1CN([C@H]1CC[C@@H](C(=O)Nc3ccc(C)c(OC)c3)CC1)C2=O] |
| 157 | 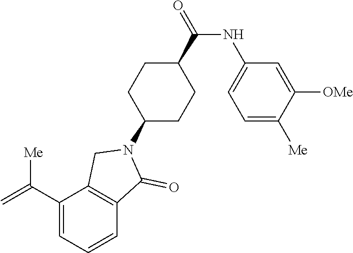 [SMILES: C=C(C)c1cccc2c1CN([C@H]1CC[C@@H](C(=O)Nc3ccc(C)c(OC)c3)CC1)C2=O] |
| 158 | 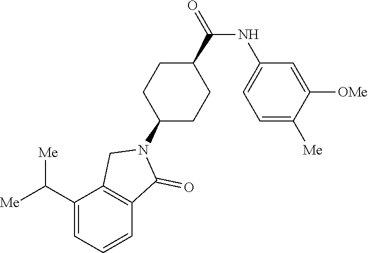 [SMILES: COc1cc(NC(=O)[C@H]2CC[C@@H](N3Cc4c(cccc4C(C)C)C3=O)CC2)ccc1C] |
| 159 | 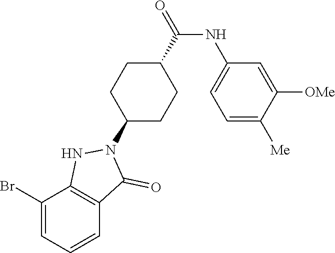 [SMILES: COc1cc(NC(=O)[C@H]2CC[C@H](n3[nH]c4c(Br)cccc4c3=O)CC2)ccc1C] |
| 160 | 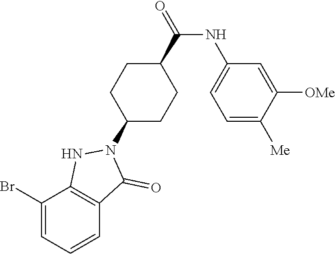 [SMILES: COc1cc(NC(=O)[C@H]2CC[C@@H](n3[nH]c4c(Br)cccc4c3=O)CC2)ccc1C] |
| 161 | 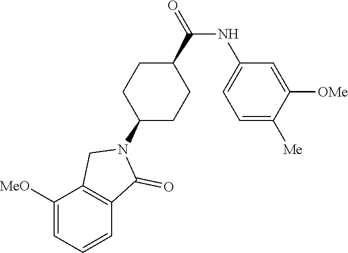 [SMILES: COc1cc(NC(=O)[C@H]2CC[C@@H](N3Cc4c(OC)cccc4C3=O)CC2)ccc1C] |
| 162 | 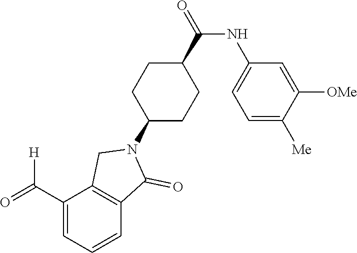 [SMILES: COc1cc(NC(=O)[C@H]2CC[C@@H](N3Cc4c(C=O)cccc4C3=O)CC2)ccc1C] |
| 163 | 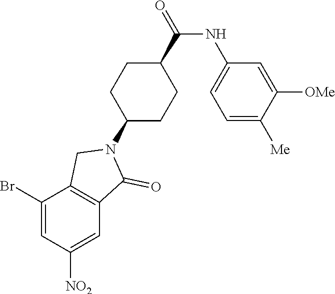 [SMILES: COc1cc(NC(=O)[C@H]2CC[C@@H](N3Cc4c(Br)cc([N+](=O)[O-])cc4C3=O)CC2)ccc1C] |
| 164 | 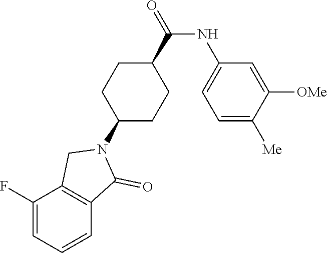 [SMILES: COc1cc(NC(=O)[C@H]2CC[C@@H](N3Cc4c(F)cccc4C3=O)CC2)ccc1C] |
| 165 | 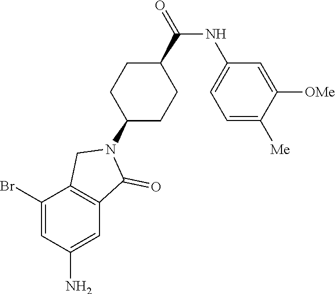 [SMILES: COc1cc(NC(=O)[C@H]2CC[C@@H](N3Cc4c(Br)cc(N)cc4C3=O)CC2)ccc1C] |
| 166 | 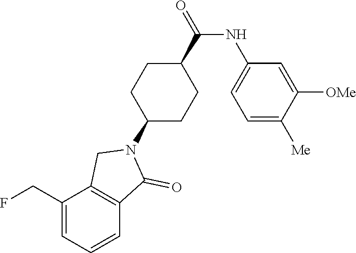 [SMILES: COc1cc(NC(=O)[C@H]2CC[C@@H](N3Cc4c(CF)cccc4C3=O)CC2)ccc1C] |
| 167 | 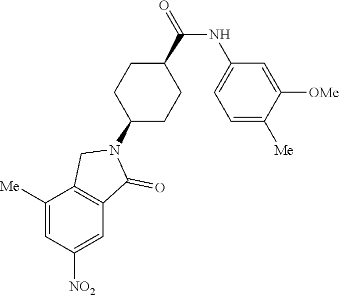 [SMILES: COc1cc(NC(=O)[C@H]2CC[C@@H](N3Cc4c(C)cc([N+](=O)[O-])cc4C3=O)CC2)ccc1C] |
| 168 | 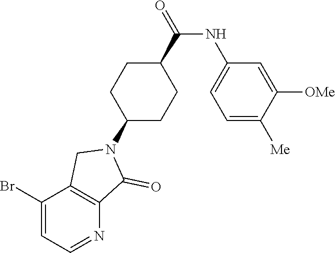 [SMILES: COc1cc(NC(=O)[C@H]2CC[C@@H](N3Cc4c(Br)ccnc4C3=O)CC2)ccc1C] |
| 169 | 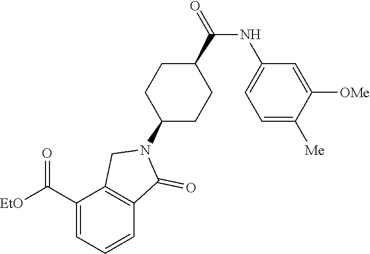 [SMILES: CCOC(=O)c1cccc2c1CN([C@H]1CC[C@@H](C(=O)Nc3ccc(C)c(OC)c3)CC1)C2=O] |
| 170 | 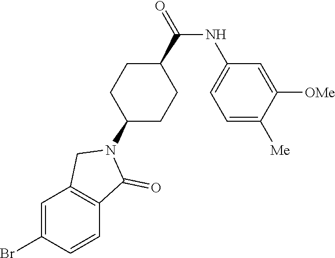 [SMILES: COc1cc(NC(=O)[C@H]2CC[C@@H](N3Cc4cc(Br)ccc4C3=O)CC2)ccc1C] |
| 171 | 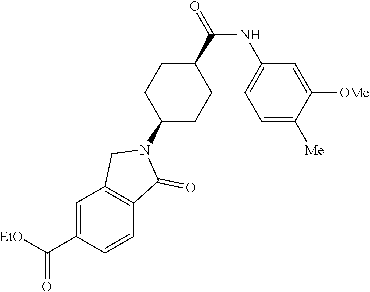 [SMILES: CCOC(=O)c1ccc2c(c1)CN([C@H]1CC[C@@H](C(=O)Nc3ccc(C)c(OC)c3)CC1)C2=O] |
| 172 | 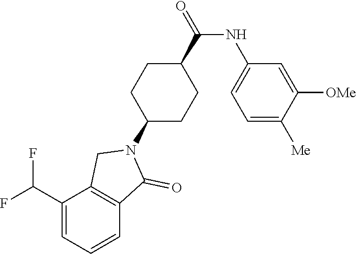 [SMILES: COc1cc(NC(=O)[C@H]2CC[C@@H](N3Cc4c(cccc4C(F)F)C3=O)CC2)ccc1C] |
| 173 | 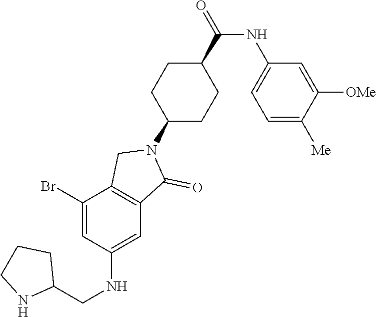 [SMILES: COc1cc(NC(=O)[C@H]2CC[C@@H](N3Cc4c(Br)cc(NCC5CCCN5)cc4C3=O)CC2)ccc1C] |
| 174 | 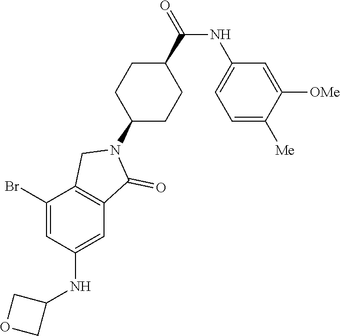 [SMILES: COc1cc(NC(=O)[C@H]2CC[C@@H](N3Cc4c(Br)cc(NC5COC5)cc4C3=O)CC2)ccc1C] |
| 175 | 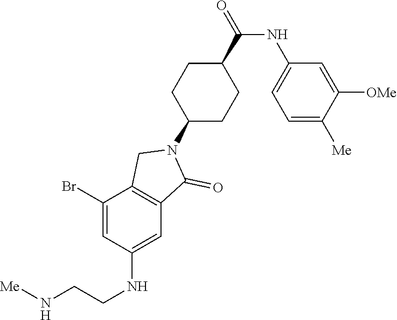 [SMILES: CNCCNc1cc(Br)c2c(c1)C(=O)N([C@H]1CC[C@@H](C(=O)Nc3ccc(C)c(OC)c3)CC1)C2] |
| 176 | 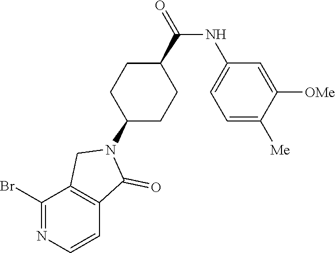 [SMILES: COc1cc(NC(=O)[C@H]2CC[C@@H](N3Cc4c(ccnc4Br)C3=O)CC2)ccc1C] |
| 177 | 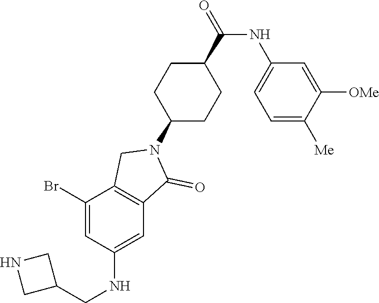 [SMILES: COc1cc(NC(=O)[C@H]2CC[C@@H](N3Cc4c(Br)cc(NCC5CNC5)cc4C3=O)CC2)ccc1C] |
| 178 | 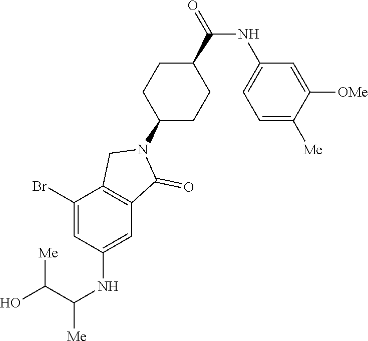 [SMILES: COc1cc(NC(=O)[C@H]2CC[C@@H](N3Cc4c(Br)cc(NC(C)C(C)O)cc4C3=O)CC2)ccc1C] |
| 179 | 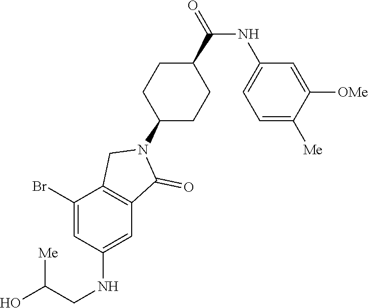 [SMILES: COc1cc(NC(=O)[C@H]2CC[C@@H](N3Cc4c(Br)cc(NCC(C)O)cc4C3=O)CC2)ccc1C] |
| 180 | 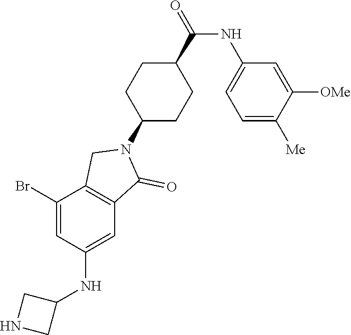 [SMILES: COc1cc(NC(=O)[C@H]2CC[C@@H](N3Cc4c(Br)cc(NC5CNC5)cc4C3=O)CC2)ccc1C] |
| 181 | 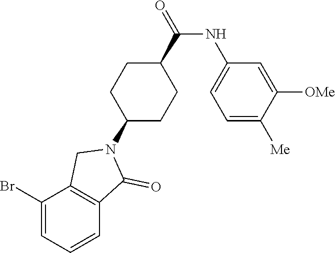 [SMILES: COc1cc(NC(=O)[C@H]2CC[C@@H](N3Cc4c(Br)cccc4C3=O)CC2)ccc1C] |
| 182 | 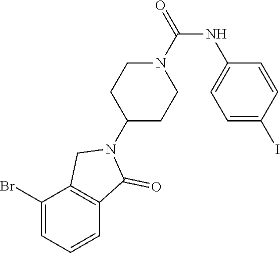 [SMILES: O=C(Nc1ccc(I)cc1)N1CCC(N2Cc3c(Br)cccc3C2=O)CC1] |
| 183 | 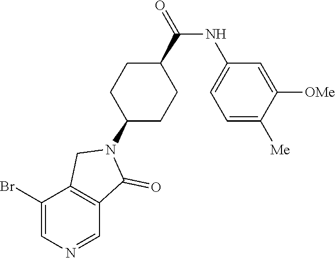 [SMILES: COc1cc(NC(=O)[C@H]2CC[C@@H](N3Cc4c(Br)cncc4C3=O)CC2)ccc1C] |
| 184 | 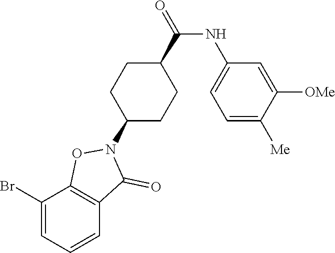 [SMILES: COc1cc(NC(=O)[C@H]2CC[C@@H](n3oc4c(Br)cccc4c3=O)CC2)ccc1C] |
| 185 | 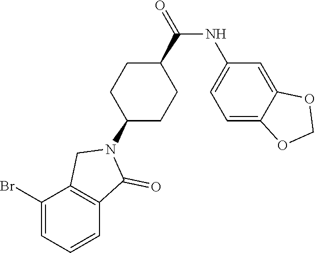 [SMILES: O=C1c2cccc(Br)c2CN1[C@H]1CC[C@@H](C(=O)Nc2ccc3c(c2)OCO3)CC1] |
| 186 | 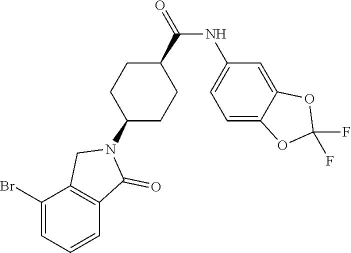 [SMILES: O=C1c2cccc(Br)c2CN1[C@H]1CC[C@@H](C(=O)Nc2ccc3c(c2)OC(F)(F)O3)CC1] |
| 187 | 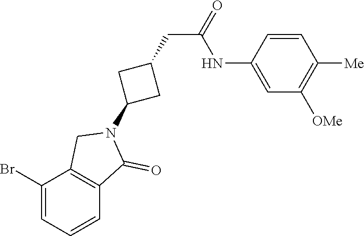 [SMILES: COc1cc(NC(=O)C[C@H]2C[C@H](N3Cc4c(Br)cccc4C3=O)C2)ccc1C] |
| 188 | 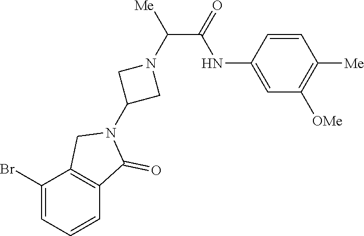 [SMILES: COc1cc(NC(=O)C(C)N2CC(N3Cc4c(Br)cccc4C3=O)C2)ccc1C] |
| 189 | 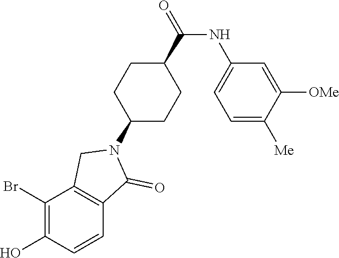 [SMILES: COc1cc(NC(=O)[C@H]2CC[C@@H](N3Cc4c(ccc(O)c4Br)C3=O)CC2)ccc1C] |
| 190 | 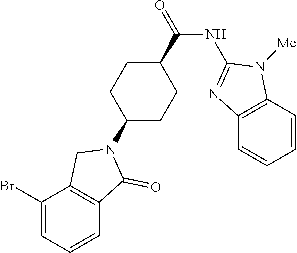 [SMILES: Cn1c(NC(=O)[C@H]2CC[C@@H](N3Cc4c(Br)cccc4C3=O)CC2)nc2ccccc21] |
| 191 | 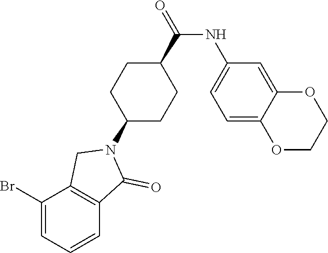 [SMILES: O=C1c2cccc(Br)c2CN1[C@H]1CC[C@@H](C(=O)Nc2ccc3c(c2)OCCO3)CC1] |
| 192 | 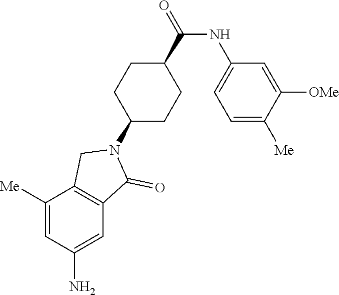 [SMILES: COc1cc(NC(=O)[C@H]2CC[C@@H](N3Cc4c(C)cc(N)cc4C3=O)CC2)ccc1C] |
| 193 | 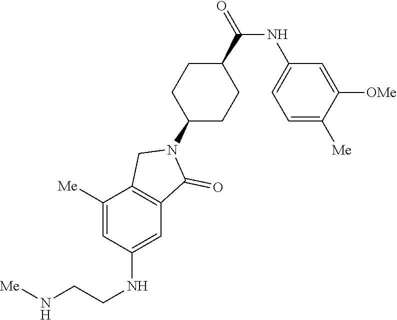 [SMILES: CNCCNc1cc(C)c2c(c1)C(=O)N([C@H]1CC[C@@H](C(=O)Nc3ccc(C)c(OC)c3)CC1)C2] |
| 194 | 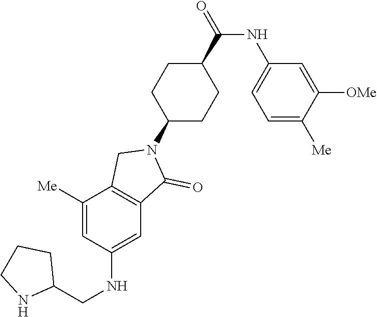 [SMILES: COc1cc(NC(=O)[C@H]2CC[C@@H](N3Cc4c(C)cc(NCC5CCCN5)cc4C3=O)CC2)ccc1C] |
| 195 | 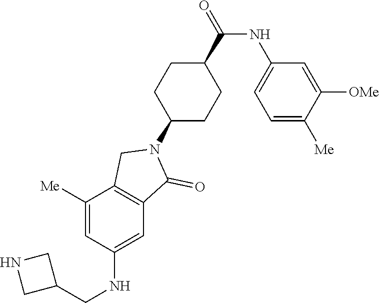 [SMILES: COc1cc(NC(=O)[C@H]2CC[C@@H](N3Cc4c(C)cc(NCC5CNC5)cc4C3=O)CC2)ccc1C] |
| 196 | 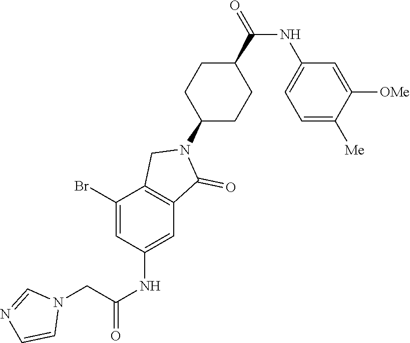 [SMILES: COc1cc(NC(=O)[C@H]2CC[C@@H](N3Cc4c(Br)cc(NC(=O)Cn5ccnc5)cc4C3=O)CC2)ccc1C] |
| 197 | 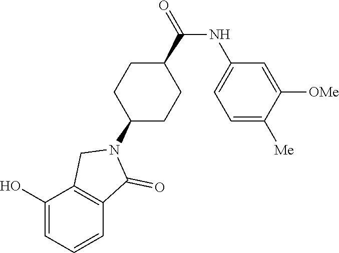 [SMILES: COc1cc(NC(=O)[C@H]2CC[C@@H](N3Cc4c(O)cccc4C3=O)CC2)ccc1C] |
| 198 | 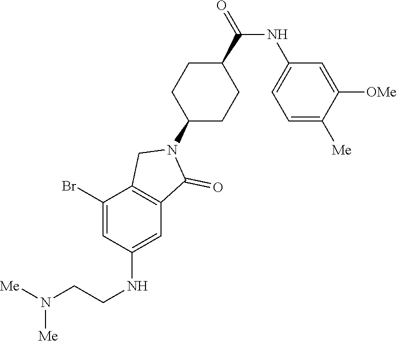 [SMILES: COc1cc(NC(=O)[C@H]2CC[C@@H](N3Cc4c(Br)cc(NCCN(C)C)cc4C3=O)CC2)ccc1C] |
| 199 | 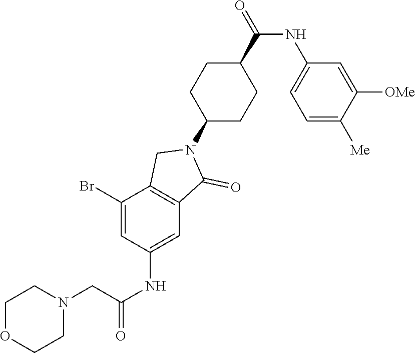 [SMILES: COc1cc(NC(=O)[C@H]2CC[C@@H](N3Cc4c(Br)cc(NC(=O)CN5CCOCC5)cc4C3=O)CC2)ccc1C] |
| 200 | 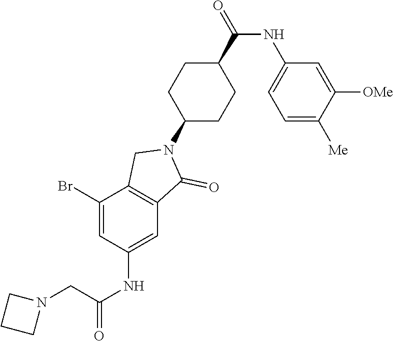 [SMILES: COc1cc(NC(=O)[C@H]2CC[C@@H](N3Cc4c(Br)cc(NC(=O)CN5CCC5)cc4C3=O)CC2)ccc1C] |
| 201 | 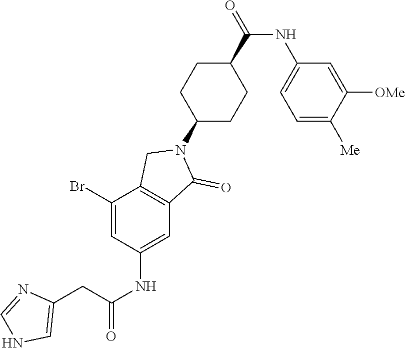 [SMILES: COc1cc(NC(=O)[C@H]2CC[C@@H](N3Cc4c(Br)cc(NC(=O)Cc5c[nH]cn5)cc4C3=O)CC2)ccc1C] |
| 202 | 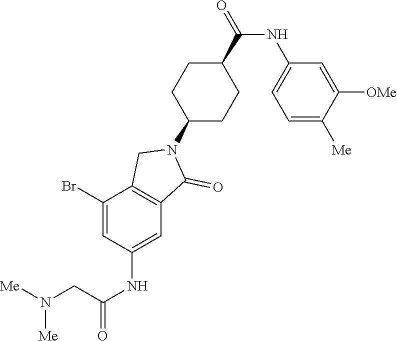 [SMILES: COc1cc(NC(=O)[C@H]2CC[C@@H](N3Cc4c(Br)cc(NC(=O)CN(C)C)cc4C3=O)CC2)ccc1C] |
| 203 | 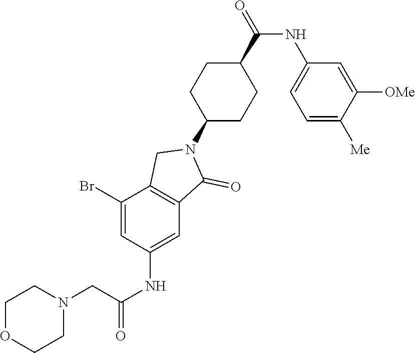 [SMILES: COc1cc(NC(=O)[C@H]2CC[C@@H](N3Cc4c(Br)cc(NC(=O)CN5CCOCC5)cc4C3=O)CC2)ccc1C] |
| 204 | 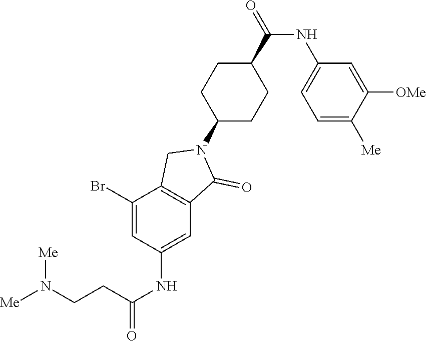 [SMILES: COc1cc(NC(=O)[C@H]2CC[C@@H](N3Cc4c(Br)cc(NC(=O)CCN(C)C)cc4C3=O)CC2)ccc1C] |
| 205 | 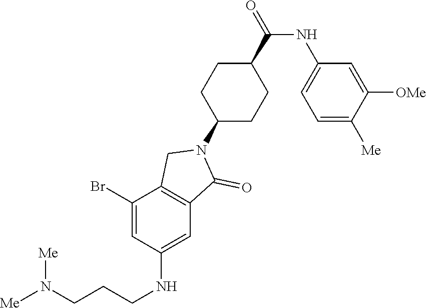 [SMILES: COc1cc(NC(=O)[C@H]2CC[C@@H](N3Cc4c(Br)cc(NCCCN(C)C)cc4C3=O)CC2)ccc1C] |
| 206 | 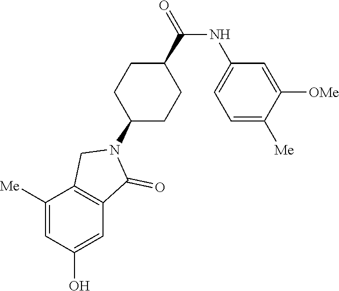 [SMILES: COc1cc(NC(=O)[C@H]2CC[C@@H](N3Cc4c(C)cc(O)cc4C3=O)CC2)ccc1C] |
| 207 | 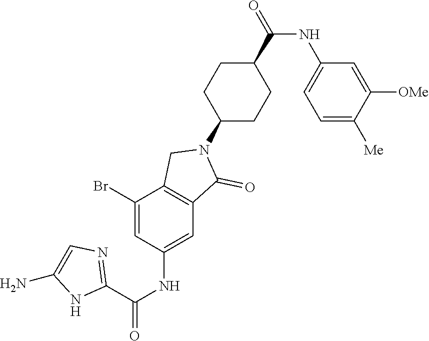 [SMILES: COc1cc(NC(=O)[C@H]2CC[C@@H](N3Cc4c(Br)cc(NC(=O)c5ncc(N)[nH]5)cc4C3=O)CC2)ccc1C] |
| 208 | 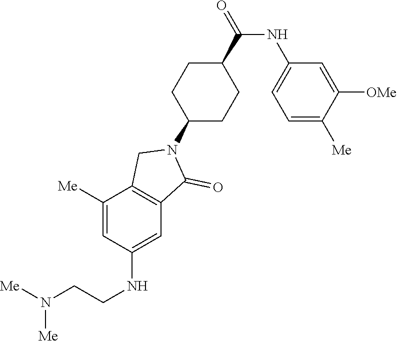 [SMILES: COc1cc(NC(=O)[C@H]2CC[C@@H](N3Cc4c(C)cc(NCCN(C)C)cc4C3=O)CC2)ccc1C] |
| 209 | 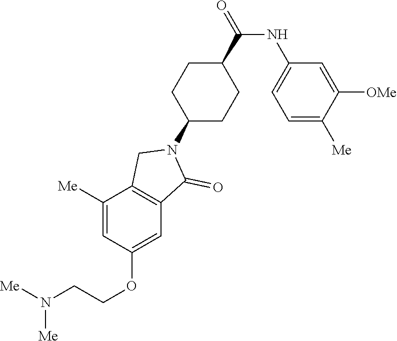 [SMILES: COc1cc(NC(=O)[C@H]2CC[C@@H](N3Cc4c(C)cc(OCCN(C)C)cc4C3=O)CC2)ccc1C] |
| 210 | 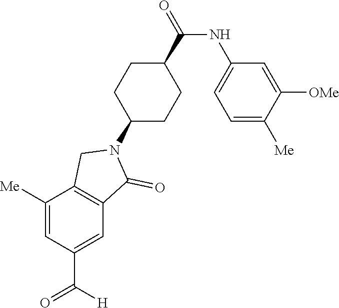 [SMILES: COc1cc(NC(=O)[C@H]2CC[C@@H](N3Cc4c(C)cc(C=O)cc4C3=O)CC2)ccc1C] |
| 211 | 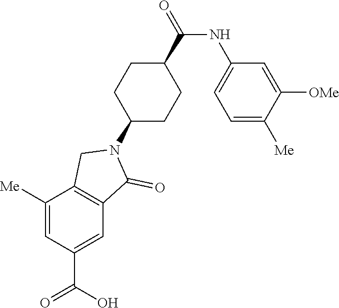 [SMILES: COc1cc(NC(=O)[C@H]2CC[C@@H](N3Cc4c(C)cc(C(=O)O)cc4C3=O)CC2)ccc1C] |
| 212 | 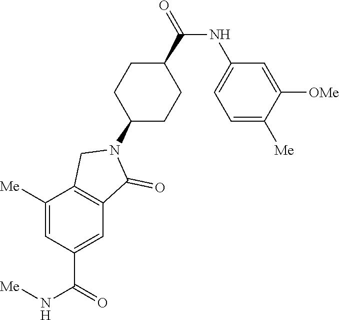 [SMILES: CNC(=O)c1cc(C)c2c(c1)C(=O)N([C@H]1CC[C@@H](C(=O)Nc3ccc(C)c(OC)c3)CC1)C2] |
| 213 | 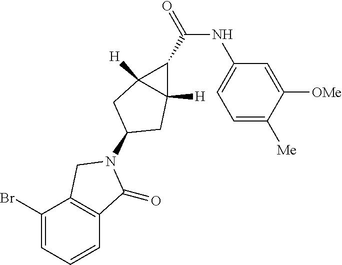 [SMILES: COc1cc(NC(=O)[C@@H]2[C@@H]3C[C@@H](N4Cc5c(Br)cccc5C4=O)C[C@@H]32)ccc1C] |
| 214 | 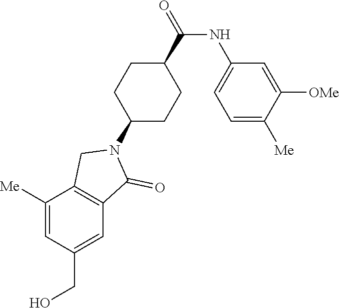 [SMILES: COc1cc(NC(=O)[C@H]2CC[C@@H](N3Cc4c(C)cc(CO)cc4C3=O)CC2)ccc1C] |
| 215 | 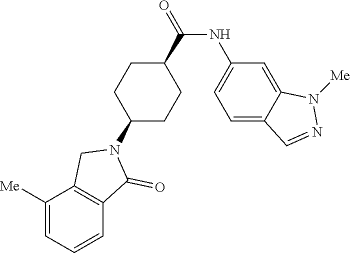 [SMILES: Cc1cccc2c1CN([C@H]1CC[C@@H](C(=O)Nc3ccc4cnn(C)c4c3)CC1)C2=O] |
| 216 | 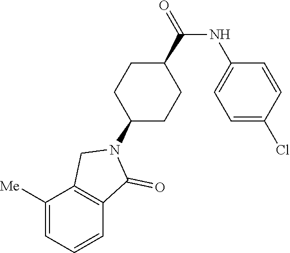 [SMILES: Cc1cccc2c1CN([C@H]1CC[C@@H](C(=O)Nc3ccc(Cl)cc3)CC1)C2=O] |
| 217 | 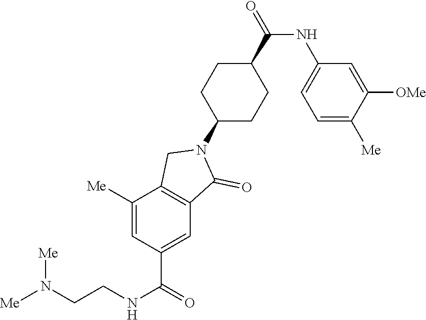 [SMILES: COc1cc(NC(=O)[C@H]2CC[C@@H](N3Cc4c(C)cc(C(=O)NCCN(C)C)cc4C3=O)CC2)ccc1C] |
| 218 | 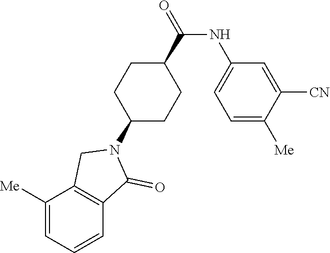 [SMILES: Cc1ccc(NC(=O)[C@H]2CC[C@@H](N3Cc4c(C)cccc4C3=O)CC2)cc1C#N] |
| 219 | 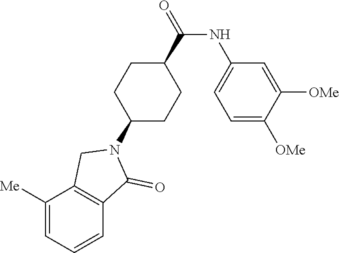 [SMILES: COc1ccc(NC(=O)[C@H]2CC[C@@H](N3Cc4c(C)cccc4C3=O)CC2)cc1OC] |
| 220 | 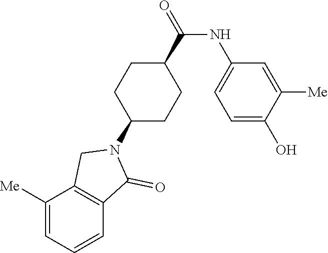 [SMILES: Cc1cc(NC(=O)[C@H]2CC[C@@H](N3Cc4c(C)cccc4C3=O)CC2)ccc1O] |
| 221 | 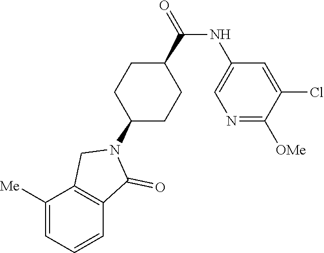 [SMILES: COc1ncc(NC(=O)[C@H]2CC[C@@H](N3Cc4c(C)cccc4C3=O)CC2)cc1Cl] |
| 222 | 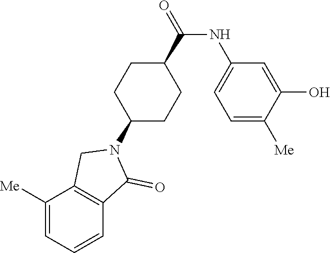 [SMILES: Cc1ccc(NC(=O)[C@H]2CC[C@@H](N3Cc4c(C)cccc4C3=O)CC2)cc1O] |
| 223 | 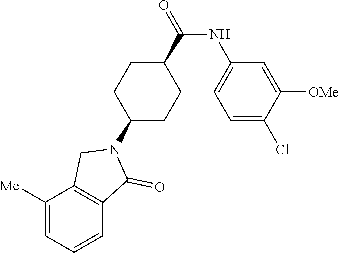 [SMILES: COc1cc(NC(=O)[C@H]2CC[C@@H](N3Cc4c(C)cccc4C3=O)CC2)ccc1Cl] |
| 224 | 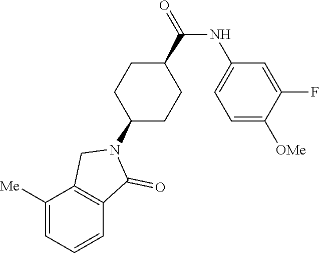 [SMILES: COc1ccc(NC(=O)[C@H]2CC[C@@H](N3Cc4c(C)cccc4C3=O)CC2)cc1F] |
| 225 | 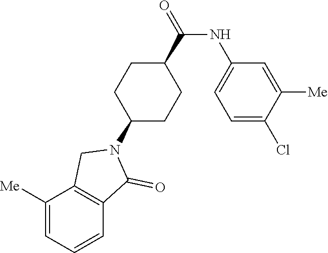 [SMILES: Cc1cc(NC(=O)[C@H]2CC[C@@H](N3Cc4c(C)cccc4C3=O)CC2)ccc1Cl] |
| 226 | 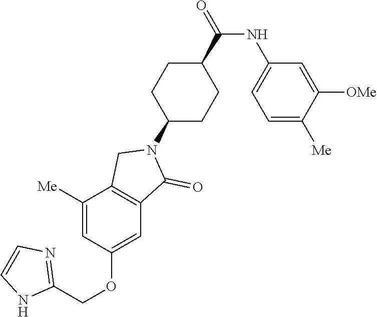 [SMILES: COc1cc(NC(=O)[C@H]2CC[C@@H](N3Cc4c(C)cc(OCc5ncc[nH]5)cc4C3=O)CC2)ccc1C] |
| 227 | 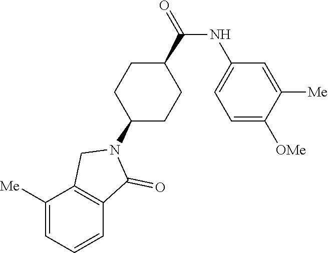 [SMILES: COc1ccc(NC(=O)[C@H]2CC[C@@H](N3Cc4c(C)cccc4C3=O)CC2)cc1C] |
| 228 | 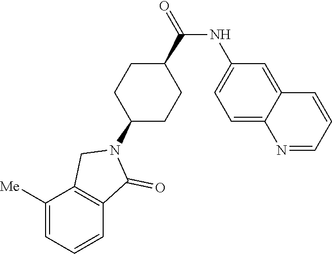 [SMILES: Cc1cccc2c1CN([C@H]1CC[C@@H](C(=O)Nc3ccc4ncccc4c3)CC1)C2=O] |
| 229 | 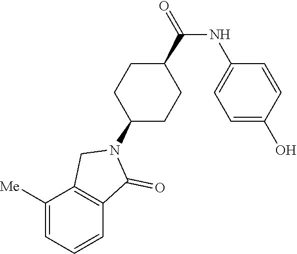 [SMILES: Cc1cccc2c1CN([C@H]1CC[C@@H](C(=O)Nc3ccc(O)cc3)CC1)C2=O] |
| 230 | 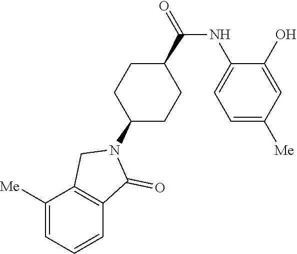 [SMILES: Cc1ccc(NC(=O)[C@H]2CC[C@@H](N3Cc4c(C)cccc4C3=O)CC2)c(O)c1] |
| 231 | 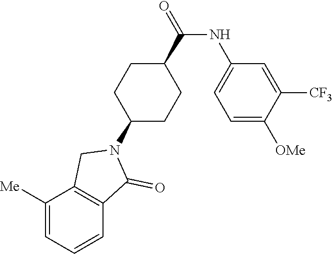 [SMILES: COc1ccc(NC(=O)[C@H]2CC[C@@H](N3Cc4c(C)cccc4C3=O)CC2)cc1C(F)(F)F] |
| 232 | 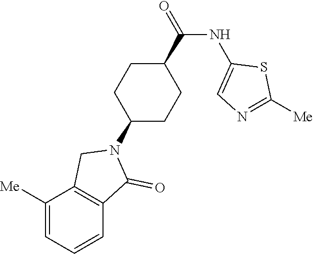 [SMILES: Cc1ncc(NC(=O)[C@H]2CC[C@@H](N3Cc4c(C)cccc4C3=O)CC2)s1] |
| 233 | 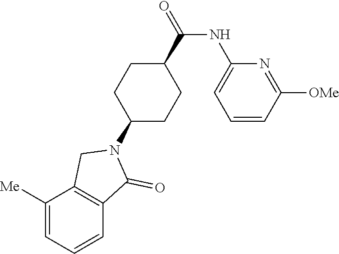 [SMILES: COc1cccc(NC(=O)[C@H]2CC[C@@H](N3Cc4c(C)cccc4C3=O)CC2)n1] |
| 234 | 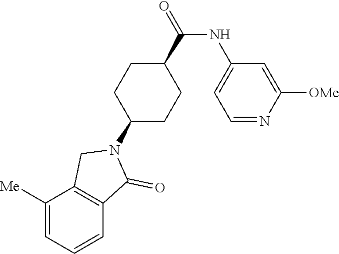 [SMILES: COc1cc(NC(=O)[C@H]2CC[C@@H](N3Cc4c(C)cccc4C3=O)CC2)ccn1] |
| 235 | 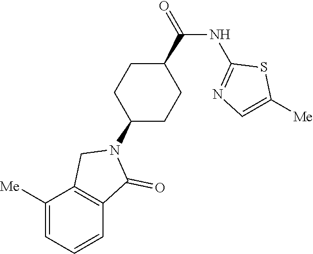 [SMILES: Cc1cnc(NC(=O)[C@H]2CC[C@@H](N3Cc4c(C)cccc4C3=O)CC2)s1] |
| 236 | 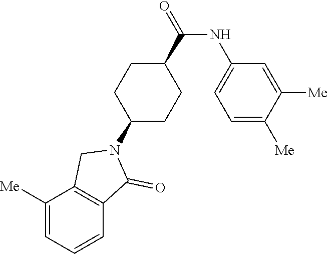 [SMILES: Cc1ccc(NC(=O)[C@H]2CC[C@@H](N3Cc4c(C)cccc4C3=O)CC2)cc1C] |
| 237 | 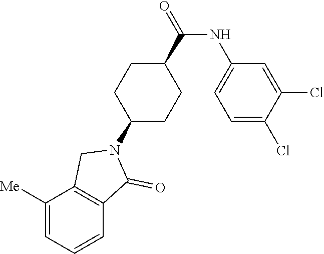 [SMILES: Cc1cccc2c1CN([C@H]1CC[C@@H](C(=O)Nc3ccc(Cl)c(Cl)c3)CC1)C2=O] |
| 238 | 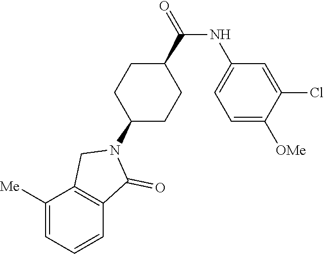 [SMILES: COc1ccc(NC(=O)[C@H]2CC[C@@H](N3Cc4c(C)cccc4C3=O)CC2)cc1Cl] |
| 239 | 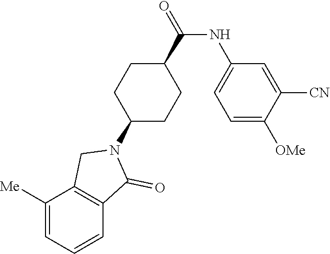 [SMILES: COc1ccc(NC(=O)[C@H]2CC[C@@H](N3Cc4c(C)cccc4C3=O)CC2)cc1C#N] |
| 240 | 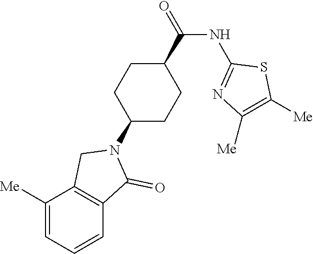 [SMILES: Cc1cccc2c1CN([C@H]1CC[C@@H](C(=O)Nc3nc(C)c(C)s3)CC1)C2=O] |
| 241 | 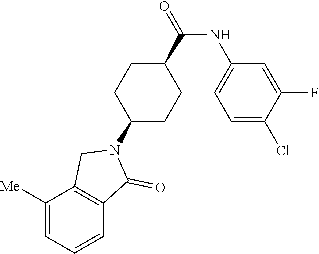 [SMILES: Cc1cccc2c1CN([C@H]1CC[C@@H](C(=O)Nc3ccc(Cl)c(F)c3)CC1)C2=O] |
| 242 | 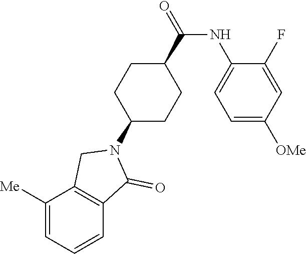 [SMILES: COc1ccc(NC(=O)[C@H]2CC[C@@H](N3Cc4c(C)cccc4C3=O)CC2)c(F)c1] |
| 243 | 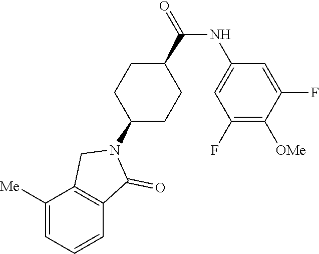 [SMILES: COc1c(F)cc(NC(=O)[C@H]2CC[C@@H](N3Cc4c(C)cccc4C3=O)CC2)cc1F] |
| 244 | 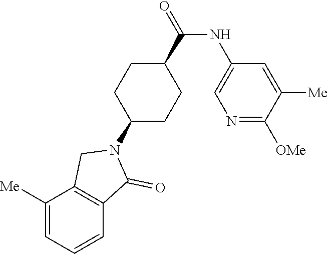 [SMILES: COc1ncc(NC(=O)[C@H]2CC[C@@H](N3Cc4c(C)cccc4C3=O)CC2)cc1C] |
| 245 | 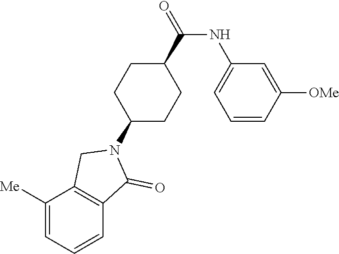 [SMILES: COc1cccc(NC(=O)[C@H]2CC[C@@H](N3Cc4c(C)cccc4C3=O)CC2)c1] |
| 246 | 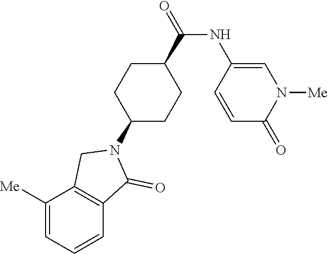 [SMILES: Cc1cccc2c1CN([C@H]1CC[C@@H](C(=O)Nc3ccc(=O)n(C)c3)CC1)C2=O] |
| 247 | 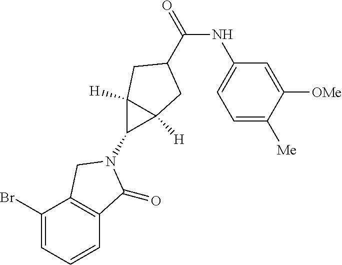 [SMILES: COc1cc(NC(=O)C2C[C@@H]3[C@H](C2)[C@H]3N2Cc3c(Br)cccc3C2=O)ccc1C] |
| 248 | 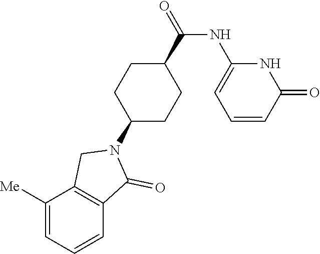 [SMILES: Cc1cccc2c1CN([C@H]1CC[C@@H](C(=O)Nc3cccc(=O)[nH]3)CC1)C2=O] |
| 249 | 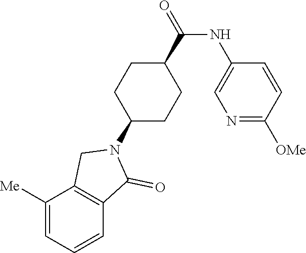 [SMILES: COc1ccc(NC(=O)[C@H]2CC[C@@H](N3Cc4c(C)cccc4C3=O)CC2)cn1] |
| 250 | 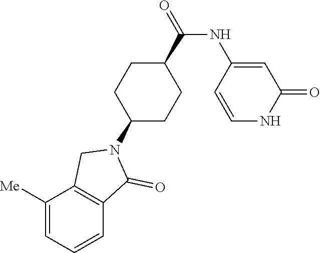 [SMILES: Cc1cccc2c1CN([C@H]1CC[C@@H](C(=O)Nc3cc[nH]c(=O)c3)CC1)C2=O] |
| 251 | 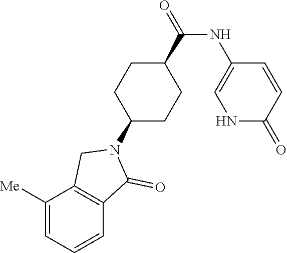 [SMILES: Cc1cccc2c1CN([C@H]1CC[C@@H](C(=O)Nc3ccc(=O)[nH]c3)CC1)C2=O] |
| 252 | 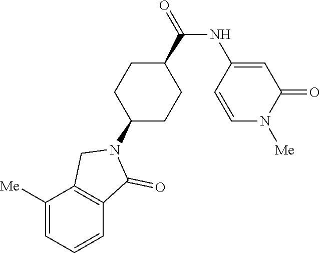 [SMILES: Cc1cccc2c1CN([C@H]1CC[C@@H](C(=O)Nc3ccn(C)c(=O)c3)CC1)C2=O] |
| 253 | 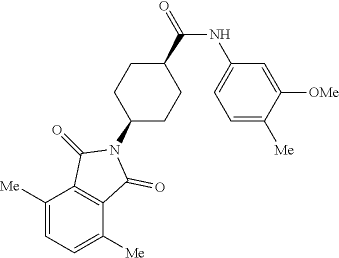 [SMILES: COc1cc(NC(=O)[C@H]2CC[C@@H](N3C(=O)c4c(C)ccc(C)c4C3=O)CC2)ccc1C] |
| 254 | 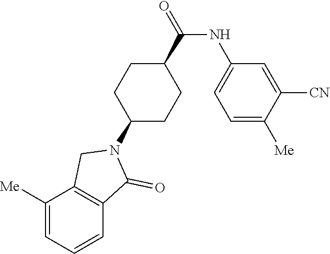 [SMILES: Cc1ccc(NC(=O)[C@H]2CC[C@@H](N3Cc4c(C)cccc4C3=O)CC2)cc1C#N] |
| 255 | 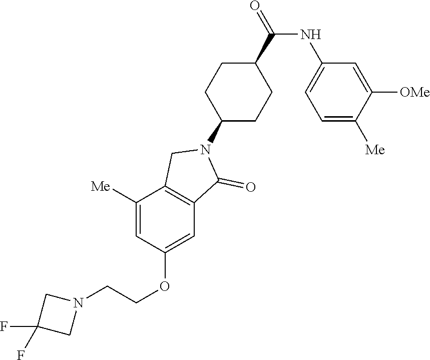 [SMILES: COc1cc(NC(=O)[C@H]2CC[C@@H](N3Cc4c(C)cc(OCCN5CC(F)(F)C5)cc4C3=O)CC2)ccc1C] |
| 256 | 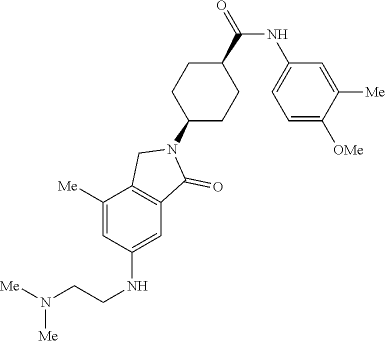 [SMILES: COc1ccc(NC(=O)[C@H]2CC[C@@H](N3Cc4c(C)cc(NCCN(C)C)cc4C3=O)CC2)cc1C] |
| 257 | 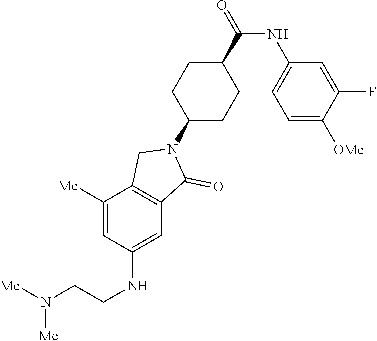 [SMILES: COc1ccc(NC(=O)[C@H]2CC[C@@H](N3Cc4c(C)cc(NCCN(C)C)cc4C3=O)CC2)cc1F] |
| 258 | 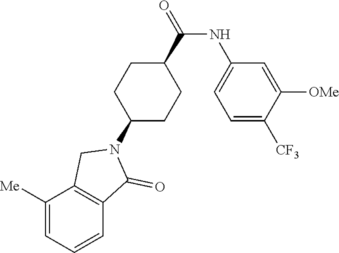 [SMILES: COc1cc(NC(=O)[C@H]2CC[C@@H](N3Cc4c(C)cccc4C3=O)CC2)ccc1C(F)(F)F] |
| 259 | 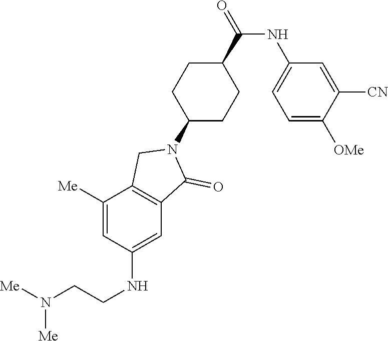 [SMILES: COc1ccc(NC(=O)[C@H]2CC[C@@H](N3Cc4c(C)cc(NCCN(C)C)cc4C3=O)CC2)cc1C#N] |
| 260 | 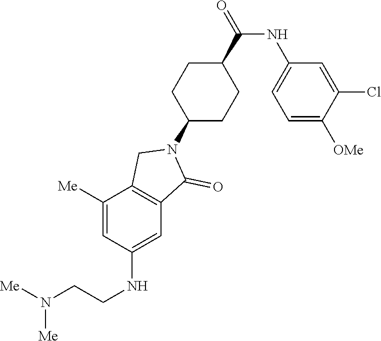 [SMILES: COc1ccc(NC(=O)[C@H]2CC[C@@H](N3Cc4c(C)cc(NCCN(C)C)cc4C3=O)CC2)cc1Cl] |
| 261 | 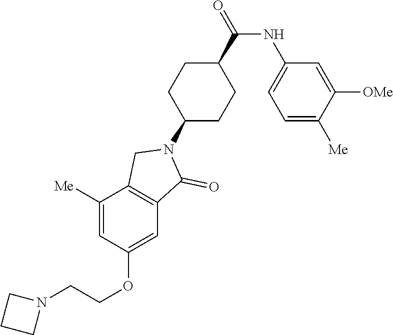 [SMILES: COc1cc(NC(=O)[C@H]2CC[C@@H](N3Cc4c(C)cc(OCCN5CCC5)cc4C3=O)CC2)ccc1C] |
| 263 | 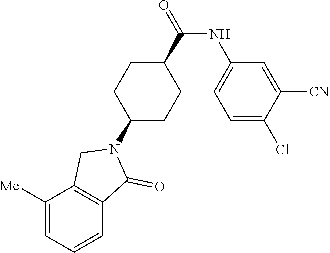 [SMILES: Cc1cccc2c1CN([C@H]1CC[C@@H](C(=O)Nc3ccc(Cl)c(C#N)c3)CC1)C2=O] |
| 264 | 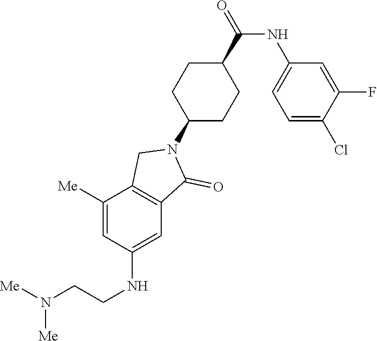 [SMILES: Cc1cc(NCCN(C)C)cc2c1CN([C@H]1CC[C@@H](C(=O)Nc3ccc(Cl)c(F)c3)CC1)C2=O] |
| 265 | 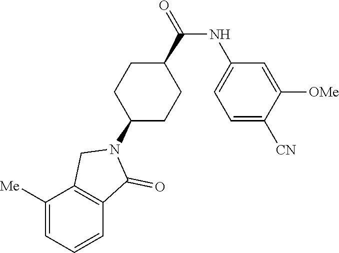 [SMILES: COc1cc(NC(=O)[C@H]2CC[C@@H](N3Cc4c(C)cccc4C3=O)CC2)ccc1C#N] |
| 266 | 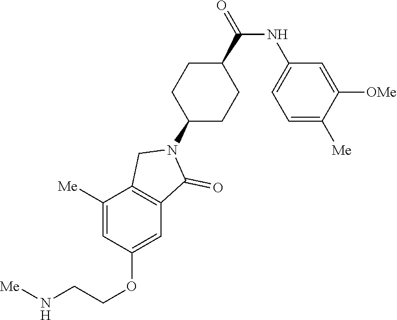 [SMILES: CNCCOc1cc(C)c2c(c1)C(=O)N([C@H]1CC[C@@H](C(=O)Nc3ccc(C)c(OC)c3)CC1)C2] |
| 267 | 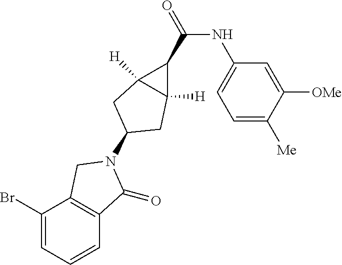 [SMILES: COc1cc(NC(=O)[C@@H]2[C@@H]3C[C@H](N4Cc5c(Br)cccc5C4=O)C[C@@H]32)ccc1C] |
| 268 | 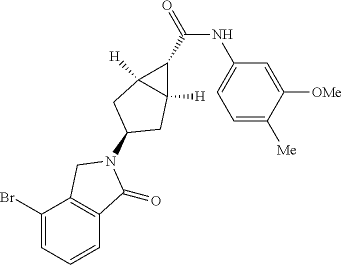 [SMILES: COc1cc(NC(=O)[C@H]2[C@@H]3C[C@H](N4Cc5c(Br)cccc5C4=O)C[C@@H]32)ccc1C] |
| 269 | 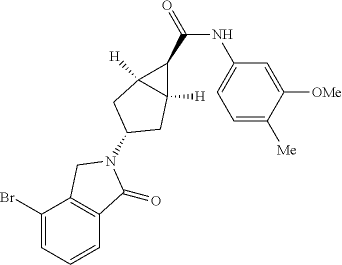 [SMILES: COc1cc(NC(=O)[C@@H]2[C@@H]3C[C@@H](N4Cc5c(Br)cccc5C4=O)C[C@@H]32)ccc1C] |
| 270 | 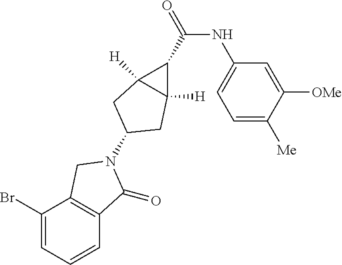 [SMILES: COc1cc(NC(=O)[C@H]2[C@@H]3C[C@@H](N4Cc5c(Br)cccc5C4=O)C[C@@H]32)ccc1C] |
| 271 | 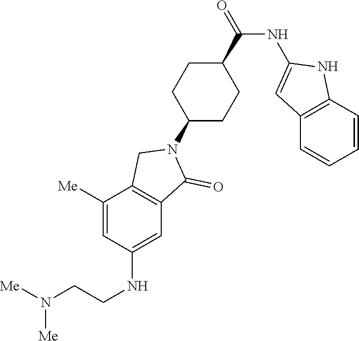 [SMILES: Cc1cc(NCCN(C)C)cc2c1CN([C@H]1CC[C@@H](C(=O)Nc3cc4ccccc4[nH]3)CC1)C2=O] |
| 272 | 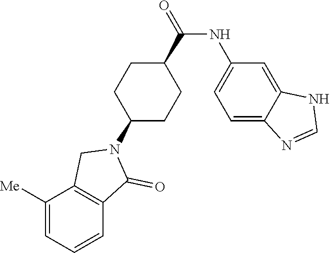 [SMILES: Cc1cccc2c1CN([C@H]1CC[C@@H](C(=O)Nc3ccc4nc[nH]c4c3)CC1)C2=O] |
| 273 | 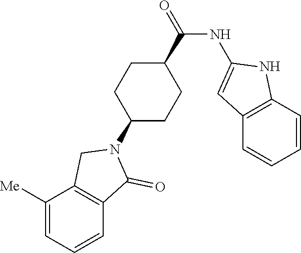 [SMILES: Cc1cccc2c1CN([C@H]1CC[C@@H](C(=O)Nc3cc4ccccc4[nH]3)CC1)C2=O] |
| 274 | 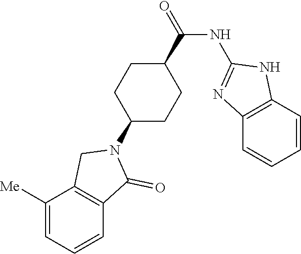 [SMILES: Cc1cccc2c1CN([C@H]1CC[C@@H](C(=O)Nc3nc4ccccc4[nH]3)CC1)C2=O] |
| 275 | 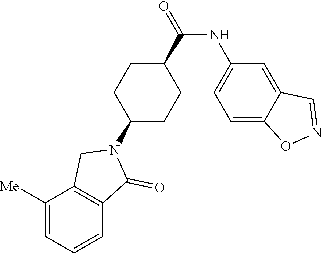 [SMILES: Cc1cccc2c1CN([C@H]1CC[C@@H](C(=O)Nc3ccc4oncc4c3)CC1)C2=O] |
| 276 | 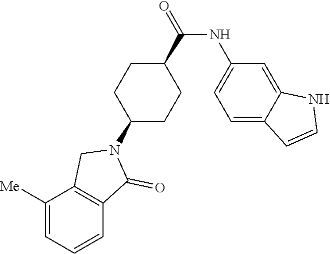 [SMILES: Cc1cccc2c1CN([C@H]1CC[C@@H](C(=O)Nc3ccc4cc[nH]c4c3)CC1)C2=O] |
| 277 | 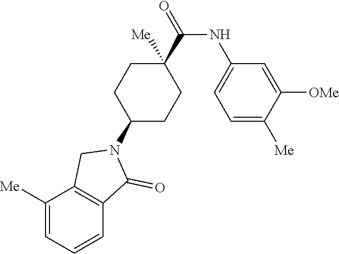 [SMILES: COc1cc(NC(=O)[C@]2(C)CC[C@H](N3Cc4c(C)cccc4C3=O)CC2)ccc1C] |
| 278 | 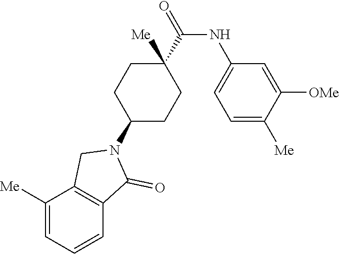 [SMILES: COc1cc(NC(=O)[C@]2(C)CC[C@@H](N3Cc4c(C)cccc4C3=O)CC2)ccc1C] |

### Biological Testing Methods

### Biological Example 1: Biochemical Assay to Measure Enzymatic Activity of hOGG1 (OGG1/APEX IC₅₀)

The reported fluorescence-based assay (Donley et al ACS Chem Biol 2015; 10 (10), 2334-2343) was modified to include hAPE1/APEX1 protein. The oligonucleotide substrate with 8-oxo-dG lesion at X was prepared (LGC Biosearch, Petaluma, CA):

5' TAMRA-TCA CCX TCG TAC GAC TC
3' BHQ2---AGT GGC AGC ATG CTG AG

Protein His-hOGG1 (12-323) was prepared by Chempartner Corporation via E. coliexpression and purification by Ni-NTA and S200 column to afford 95% purity by SDS-PAGE and SEC-HPLC analysis and hAPE1/APEX1 was purchased (SinoBiological). The assay was run in 384-well plates following the general procedure: (1) compound dilution; (2) addition of hOGG1/APEX1 to compound wells; (3) addition of DNA substrate to compound wells; (4) read Ex520/Em600 (TAMRA) fluorescence signal after 40 minutes; (5) calculate % inhibition/compound concentration; (6) calculate dose-response curve and IC50 value.

### Compound Dilution:

(1) 20 mM of compound solution was prepared in DMSO;
(2) Compounds were diluted to 10 mM in DMSO in ECHO Source plate (Labcyte PP-0200), by mixing 25 mL stock solution with 25 mL DMSO;
(3) 4× dilution of compounds, for 10 points, by mixing 10 mL solution with 30 mL DMSO; and
(4) Dispensed 150 nL compounds to assay plate (Corning 3573) in column 3-22. Dispensed 150 nL DMSO to Min and Max control wells in column 1-2 and column 23-24.

### Fluorescence Assay:

(1) Assay buffer was prepared (20 mM Tris pH 7.5, 100 mM KCl, 0.1% BSA, 0.01% Tween).
(2) 15 mL buffer was added to Min control wells (Column 1-2).
(3) Enzyme was diluted to 2× with buffer (2 nM OGG1+4 nM APEX1). Dispensed 15 mL in assay wells (except Min control wells). Shake at 500 rpm at RT for 20 min.
(4) DNA substrate diluted to 2× with buffer (12 nM). Dispensed 15 mL in all wells. Final concentration: 1 nM OGG1+2 nM APEX+6 nM dsDNA. Shake at RT for 40 min.
(5) Ex520/Em600 (TAMRA) was read.

### Data Handling:

(1) Calculated respective mean of Max and Min control wells.
(2) Converted data to percentage inhibition.

Inhibition (%)=(Max mean−value)/(Max mean−Min mean)×100%
(3) Calculated sigmoidal curve (3-parameter) and IC₅₀ (using GraphPad Prism software).

### Results

Results of the example compounds from Table 3 are shown in Tables 4A, 4B, and 4C. The table legend for the OGG1/APEX IC₅₀ Ranges are as follows:
+++IC₅₀<100 nM
++100 nM<IC₅₀≤1000 nM
+1000 nM<IC₅₀≤10000 nM
−10000 nM<IC₅₀

TABLE 4A

| | OGG1/APEX |
|---|---|
| Example | IC₅₀ (nM) |
| 1 | + |
| 2 | ++ |
| 3 | ++ |
| 4 | ++ |
| 5 | ++ |
| 7 | +++ |
| 9 | +++ |
| 11 | +++ |
| 13 | ++ |
| 14 | ++ |
| 15 | + |
| 16 | +++ |
| 17 | +++ |
| 18 | +++ |
| 19 | +++ |
| 20 | +++ |
| 21 | +++ |
| 22 | +++ |
| 23 | ++ |
| 24 | +++ |
| 25 | +++ |
| 26 | +++ |
| 27 | +++ |
| 28 | +++ |
| 29 | +++ |
| 30 | +++ |
| 31 | +++ |
| 32 | +++ |
| 33 | +++ |
| 34 | ++ |
| 35 | ++ |
| 36 | + |
| 37 | +++ |
| 38 | +++ |
| 39 | +++ |
| 40 | + |
| 41 | +++ |
| 42 | ++ |
| 43 | +++ |
| 44 | +++ |
| 45 | +++ |
| 46 | + |
| 47 | +++ |
| 48 | + |
| 49 | ++ |
| 50 | + |
| 51 | ++ |
| 52 | +++ |
| 53 | + |
| 54 | +++ |
| 55 | +++ |
| 56 | ++ |
| 57 | + |
| 58 | +++ |
| 59 | +++ |
| 60 | +++ |
| 61 | +++ |
| 62 | +++ |
| 63 | + |
| 64 | ++ |
| 65 | + |
| 66 | ++ |
| 67 | ++ |
| 68 | ++ |
| 69 | +++ |
| 70 | ++ |
| 71 | + |
| 72 | ++ |
| 73 | ++ |
| 74 | ++ |
| 75 | ++ |
| 76 | +++ |
| 77 | +++ |
| 78 | +++ |
| 79 | ++ |
| 80 | +++ |
| 81 | +++ |
| 82 | +++ |
| 83 | ++ |
| 84 | +++ |
| 85 | ++ |
| 86 | +++ |
| 87 | +++ |
| 88 | +++ |
| 89 | ++ |
| 90 | ++ |
| 91 | +++ |
| 92 | +++ |
| 93 | ++ |
| 94 | ++ |
| 95 | ++ |
| 96 | +++ |
| 97 | +++ |
| 98 | +++ |
| 99 | +++ |
| 100 | +++ |
| 101 | +++ |
| 102 | + |
| 103 | +++ |
| 104 | + |
| 105 | +++ |
| 106 | + |
| 107 | +++ |
| 108 | − |
| 109 | + |

TABLE 4B

| | OGG1/APEX |
|---|---|
| Example | IC₅₀ (nM) |
| 110 | + |
| 111 | − |
| 112 | +++ |
| 113 | ++ |
| 114 | + |
| 115 | ++ |
| 116 | ++ |
| 117 | ++ |
| 118 | +++ |
| 119 | ++ |
| 120 | ++ |
| 121 | + |
| 122 | ++ |
| 123 | ++ |
| 124 | ++ |
| 125 | − |
| 126 | + |
| 127 | + |
| 128 | ++ |
| 129 | ++ |
| 130 | + |
| 131 | + |
| 132 | +++ |
| 133 | − |
| 134 | − |
| 135 | +++ |
| 136 | +++ |
| 137 | +++ |
| 138 | +++ |
| 139 | − |
| 140 | +++ |
| 141 | +++ |
| 142 | ++ |
| 143 | +++ |
| 144 | +++ |
| 145 | + |
| 146 | +++ |
| 147 | + |
| 148 | + |
| 149 | ++ |
| 150 | ++ |
| 151 | + |
| 152 | - |
| 153 | ++ |
| 154 | + |
| 155 | ++ |
| 156 | + |
| 157 | − |
| 158 | − |
| 159 | − |
| 160 | ++ |
| 161 | + |
| 162 | + |
| 163 | ++ |
| 164 | + |
| 165 | ++ |
| 166 | + |
| 167 | +++ |
| 168 | ++ |
| 169 | − |
| 170 | − |
| 171 | − |
| 172 | + |
| 173 | +++ |
| 174 | ++ |
| 175 | +++ |
| 176 | ++ |
| 177 | +++ |
| 178 | + |
| 179 | ++ |
| 180 | +++ |
| 181 | + |
| 182 | − |
| 183 | ++ |
| 184 | − |
| 185 | + |
| 186 | − |
| 187 | − |
| 188 | − |
| 189 | ++ |
| 190 | + |
| 191 | + |
| 192 | +++ |
| 193 | +++ |
| 194 | +++ |
| 195 | +++ |
| 196 | ++ |
| 197 | + |
| 198 | +++ |
| 199 | ++ |
| 200 | ++ |
| 201 | ++ |
| 202 | ++ |
| 203 | ++ |
| 204 | +++ |
| 205 | +++ |
| 206 | +++ |
| 207 | ++ |
| 208 | +++ |
| 209 | +++ |
| 210 | ++ |
| 211 | ++ |
| 212 | ++ |
| 213 | + |
| 214 | ++ |
| 215 | + |
| 216 | ++ |
| 217 | +++ |

TABLE 4C

| | OGG1/APEX |
|---|---|
| Example | IC₅₀ (nM) |
| 218 | + |
| 219 | ++ |
| 220 | ++ |
| 221 | ++ |
| 222 | + |
| 223 | ++ |
| 224 | ++ |
| 225 | ++ |
| 226 | ++ |
| 227 | ++ |
| 228 | + |
| 229 | + |
| 230 | + |
| 231 | ++ |
| 232 | − |
| 233 | + |
| 234 | − |
| 235 | − |
| 236 | + |
| 237 | ++ |
| 238 | ++ |
| 239 | ++ |
| 240 | − |
| 241 | ++ |
| 242 | + |
| 243 | ++ |
| 244 | ++ |
| 245 | + |
| 246 | − |
| 247 | − |
| 248 | − |
| 249 | + |
| 250 | − |
| 251 | − |
| 252 | − |
| 253 | ++ |
| 254 | ++ |
| 255 | ++ |
| 256 | +++ |
| 257 | +++ |
| 258 | ++ |
| 259 | +++ |
| 260 | +++ |
| 261 | +++ |
| 263 | ++ |
| 264 | +++ |
| 265 | − |
| 266 | +++ |
| 267 | − |
| 268 | ++ |
| 269 | + |
| 270 | − |
| 271 | +++ |
| 272 | + |
| 273 | ++ |
| 274 | ++ |
| 275 | ++ |
| 276 | + |
| 277 | ++ |
| 278 | + |

### Biological Example 2: Cellular Thermal Shift Assay to Measure Compound Affinity for ^{nLuc}OGG1 in HeLa Cells (CETSA DTₘ)

Cell Culture and Transfection of Hela Cells with ^{nLuc}OGG1: For all tests, Hela cells were maintained in growth medium, Dulbecco's Minimum Eagle Medium (Multicell: Cat #319-005-CL) supplemented with 10% heat-inactivated fetal bovine serum (Gibco: Cat #12484-028) at 37° C. and 5% CO₂ in a humidified tissue culture incubator. Transient transfections were performed using the Lonza 4-D Nucleofector X unit according to the manufacturer's protocol (Lonza Cat #V4XC-1024). Briefly, for each transfection, 1.2×10⁶ cells were centrifuged at 1000 RPM for 5 minutes at room temperature before being diluted in 100 μL SE solution containing 18% v/v supplement and 1.5 μg pNF1-N-empty or pNF1-N-OGG1a vector. The total volume was transferred to a 100 μL nucleofection cuvette prior to transfer to the apparatus where HeLa cell nucleofection protocol CN-114 was executed. Following nucleofection, 500 μL complete media was added to the cells in the cuvette before transferring to a 6-well dish containing 1.5 mL of complete media. Cells were left to incubate for 48 hours before performing all cellular thermal shift experiments.

Cellular Thermal Shift Assays for Target Engagement (CETSA): Forty-eight hours following nucleofection, cells were washed once with DPBS−/−(Multicell: Cat #311-425-CL) and lifted with 0.25% Trypsin-EDTA (Multicell: Cat #325-043-CL) prior to dilution in growth medium and centrifugation at 1000 RPM for 5 minutes at room temperature. The resulting cell pellet was then resuspended in growth medium, cells were counted and the volume adjusted to 500 k cells/mL (2×).

For delta Tm determination, OGG1 inhibitor compounds were dissolved in DMSO to a final working concentration of 20 mM. Working solutions were then diluted in complete media to 20 μM or 0.1% DMSO for the vehicle control (2×). 2×OGG1 inhibitor compound solutions or 0.1% DMSO control were then pre-dispensed into MicroAmp Optical 96-well PCR reaction plates (Applied Biosystems: Cat #4346906) at 50 μL/well. Next, 50 μL or 25 k cells/well from the 2× cell suspension was added to the pre-loaded PCR plates. All plates were prepared as duplicates, covered with adhesive plate sealers (Perkin Elmer: Cat #6050185) and left to incubate at 37° C. for 60 minutes. During incubation, sufficient NanoGlo luciferase assay reagents (Promega: Cat #N1120) were left to equilibrate to room temperature in the dark. Immediately before heating the plates, the NanoGlo substrate was mixed with the NanoGlo buffer at a 1:50 ratio. The thermal cycler (Veriti 96-Well Thermal Cycler, Thermo Fisher Scientific) was allowed to reach the designated temperature prior to placement of the PCR plate into the heating block. For Tm determination experiments, duplicate plates containing a fixed concentration of test compound (10 μM final) as well as vehicle control were prepared to perform a 12-point temperature gradient spanning 37-70° C. for 3 minutes. Therefore, two thermal cycler protocols were set up covering two 6-step temperature gradients ranging from 37-52° C. and 52-70° C. Plates were then allowed to cool at room temperature for 5 minutes after the heat treatment followed by transfer of 75 μL of the reaction into a 96-well white polystyrene microplate (Corning: Cat #CLS3903) and addition of 75 μL of the prepared NanoGlo mixture. Microplates were then left to shake in the dark for 5 minutes before total luminescence was measured on a SpectraMax IX3 plate reader. The luminescence value obtained at each temperature was normalized to the luminescence obtained at 37° C. and the ratio obtained was plotted against the temperature. A Boltzman Sigmoidal non-linear regression was applied to the resulting data to determine Tm values and the difference between the Tm value for a given OGG1 inhibitor relative to the DMSO vehicle control was the delta Tm.

Surface Plasma Resonance with hOGG1 to Measure Compound Affinity (OGG1 SPR K_{d}); All the SPR studies were performed on a Biacore T-200 instrument (Cytiva life sciences) using series S NTA sensor chip (cat #BR-1005-32, Cytiva life sciences). Recombinant his-tagged OGG1 (mol. wt. 36003; ChemPartner, Shanghai, China) was immobilized on NTA sensor chip via Ni²⁺/NTA chelation using NTA reagent kit (Cytiva life sciences; cat #289950) as per manufacturer's recommended protocol. The running buffer during immobilization contained HEPES (20 mM, pH 7.4), 150 mM NaCl, 0.5 mM TCEP (tris(2-carboxyethyl)phosphine), and 0.005% Tween-20. NTA sensor chip (experimental cell) was first conditioned by injecting regeneration solution (350 mM EDTA, flow rate 30 μL/min, 2 injections 60 each). Additional wash with 50 mM NaOH was used whenever the chip was regenerated (flow rate 30 μL/min, 1 injection of 60 s). The sensor chip was then activated with 0.5 mM NiCl₂ (flow rate 10 μL/min, 2 injection of 60 s, leading to a baseline increase of ~40 RU), and the excess Ni²⁺ was removed using immobilization buffer containing 3 mM EDTA (flow rate 10 μL/min, 1 injection of 60 s). Finally, OGG1 (diluted to 6.2 μg/mL in running buffer) was injected on the activated sensor chip at a flow rate of 10 μL/min until immobilization level reached ~6000 RU. Any loosely bound protein was removed by passing the immobilization buffer overnight at 20 μL/min. At this point, the drift in baseline was ~0.01 RU/s and the final immobilization level was ~2500 RU. Reference cell was left untreated. SPR binding experiments were performed using binding buffer (20 mM HEPES (pH 7.4), 150 mM NaCl, 0.5 mM TCEP, 0.005% Tween-20, and 2% DMSO) at a flow rate of 30 μL/min. Single-cycle kinetics approach was used for binding analysis which consisted of sequential injections of increasing concentrations of test compound (a 3-fold serial dilution with 5 concentrations; highest 750 or 1500 nM; with final DMSO concentration at 2%) over the immobilized protein without regeneration step between each sample injection. Each injection was 45 seconds long (contact time), followed by a final dissociation of 600 sec. The Biacore T-200 Evaluation software 3.1 (GE Healthcare) was used to determine kinetic parameters from SPR data. All the reported sensograms were reference subtracted and corrected for refractive index contributions from solvent using 8-point solvent correction (1.4 to 2.8% DMSO in binding buffer). Data were fit to both steady state and 1:1 kinetic model to obtain Kₒₙ, K_{off} and binding affinity (K_{d}).

### Results

Results of some example compounds from Table 3 are shown in Table 5. The table legend for the CETSA DTₘ Ranges are as follows:
+++10° C.<DTₘ
++8° C.<DTₘ≤10° C.
+6° C.<DTₘ≤8° C.
−DTₘ≤6° C.

The table legend for the OGG1 SPR K_{D} Ranges are as follows:
+++K_{D}≤10 nM
++10 nM<K_{D}≤100 nM
+100 nM<K_{D}≤1000 nM
−1000 nM<K_{D}

TABLE 5

| | CETSA | OGG1 |
|---|---|---|
| | DTₘ | SPR K_{D} |
| Example | (° C.) | (nM) |
| 2 | + | ++ |
| 3 | + | ++ |
| 4 | + | ++ |
| 5 | + | +++ |
| 6 | − | − |
| 7 | ++ | ++ |
| 8 | − | − |
| 9 | ++ | +++ |
| 10 | − | − |
| 11 | ++ | +++ |
| 12 | − | − |
| 13 | ++ | ++ |
| 14 | + | ++ |
| 15 | + | ++ |
| 16 | ++ | +++ |
| 17 | +++ | +++ |
| 19 | ++ | +++ |
| 28 | +++ | +++ |
| 30 | ++ | + |
| 35 | + | ++ |
| 41 | + | ++ |
| 44 | ++ | ++ |
| 45 | ++ | ++ |
| 47 | + | ++ |
| 58 | ++ | ++ |
| 59 | ++ | ++ |
| 67 | + | ++ |
| 92 | ++ | ++ |
| 97 | ++ | ++ |
| 99 | ++ | ++ |
| 103 | ++ | ++ |
| 132 | +++ | ++ |
| 135 | + | ++ |
| 143 | + | ++ |
| 193 | +++ | ++ |
| 227 | + | ++ |
| 239 | + | ++ |
| 255 | + | ++ |
| 256 | +++ | +++ |
| 259 | ++ | ++ |
| 260 | +++ | ++ |
| 271 | +++ | ++ |
| 277 | ++ | ++ |

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1. A compound of Formula (I): 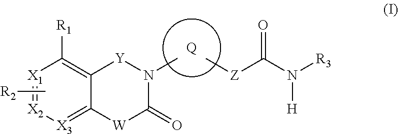
or a stereoisomer, or a pharmaceutically acceptable salt thereof, wherein:
X₁, X₂ and X₃ are each independently CH, C, or N;
R₁ is R, halo, tert-butyl, cyclopropyl, cyclobutyl, CF₃, CHF₂, CH₂F, OH, CN, CHO, C(O)Me, C(O)OMe, C(O)NH₂, CHCH₂, NR′R″, R′NR″R′″;
R₂ is absent, C₁-C₃ alkyl, C₁-C₃ alkene, NR′R″, NHR′NR″R′″, R′NR″R′″, NHC(O)R′NR″R′″, OR, CN, NO₂, C(O)R, R′OR″, R′NR″C(O)R′″, C(O)OR, C(O)NR′R″, C(O)NHR′NR″R′″, P(O)R′R″, R′NR″S(O)₂R′″, —O—R′NR″R′″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹;
Y is CR′R″, C(O), O, S, or NR;
W is CH₂, O, S, NR or a single bond;
Q is A², optionally comprising one or more of O, N, or NR;
Z is CH₂, S, O, NR or a single bond;
R₃ is A³, B², or a fused bicyclic aromatic ring system, wherein the fused bicyclic aromatic ring system optionally comprises one or more of N, NR, O, or S;
A¹, A², and A³ are each independently a 3- to 7-membered carbocyclic or heterocyclic ring;
B¹ and B² are each independently a 5- or 6-membered aromatic or heteroaromatic ring;
wherein R, R′, R″, and R′″ are each independently H, C₁₋₆ alkyl, or C₁₋₆ alkene;
wherein the R, R′, R″, R′″, A¹, A², A³, B¹, B², and the fused bicyclic aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

2. The compound of claim 1, or a stereoisomer or a pharmaceutically acceptable salt thereof, wherein the compound is of Formula (II): 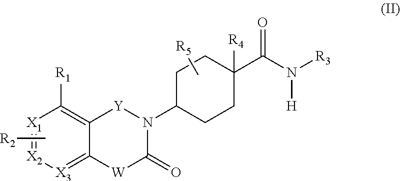
or a stereoisomer, or a pharmaceutically acceptable salt thereof, wherein:
X₁, X₂ and X₃ are independently CH, C, or N;
R₁ is R, halo, tert-butyl, cyclopropyl, cyclobutyl, CF₃, CHF₂, CH₂F, OH, CN, CHO, C(O)Me, C(O)OMe, C(O)NH₂, CHCH₂, NR′R″, R′NR″R′″;
R₂ is absent, C₁-C₃ alkyl, C₁-C₃ alkene, NR′R″, NHR′NR″R′″, R′NR″R′″, NHC(O)R′NR″R′″, OR, CN, NO₂, C(O)R, R′OR″, R′NR″C(O)R′″, C(O)OR, C(O)NR′R″, C(O)NHR′NR″R′″, P(O)R′R″, R′NR″S(O)₂R′″, —O—R′NR″R′″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹;
Y is CR′R″, C(O), O, S, or NR;
W is CH₂, O, S, NR or a single bond;
R⁵ is H₂, C₁₋₆ alkyl, C₁₋₆ alkene, OR, NR′R″, halo, or CN;
R⁴ is H, C₁₋₆ alkyl, OR, NR′R″, halo, CN;
R₃ is A³, B², or a fused bicyclic aromatic ring system, wherein the fused bicyclic aromatic ring system optionally comprises one or more of N, NR, O, or S;
A¹ and A³ are each independently a 3- to 7-membered carbocyclic or heterocyclic ring; and
B¹ and B² are each independently a 5- or 6-membered aromatic or heteroaromatic ring;
wherein R₄ and/or R₅ are optional;
wherein R, R′, R″, and R′″ are each independently H, C₁₋₆ alkyl, or C₁₋₆ alkene; and
wherein the R, R′, R″, R′″, A¹, A³, B¹, B², and the fused aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

3. The compound of claim 1, or a stereoisomer or a pharmaceutically acceptable salt thereof, wherein the compound is of Formula (IIIa) or (IIIb): 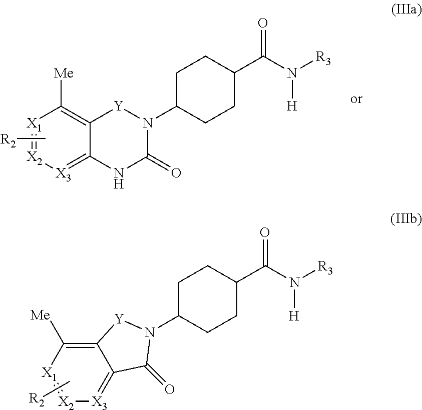
or a stereoisomer, or a pharmaceutically acceptable salt thereof, wherein:
X₁, X₂ and X₃ are independently CH, C, or N;
R₂ is absent, C₁-C₃ alkyl, C₁-C₃ alkene, NR′R″, NHR′NR″R′″, R′NR″R″′, NHC(O)R′NR″R′″, OR, CN, NO₂, C(O)R, R′OR″, R′NR″C(O)R′″, C(O)OR, C(O)NR′R″, C(O)NHR′NR″R′″, P(O)R′R″, R′NR″S(O)₂R′″, —O—R′NR″R′″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹;
Y is CR′R″, C(O), O, S, or NR;
R₃ is A³, B², or a fused bicyclic aromatic ring system, wherein the fused bicyclic aromatic ring system optionally comprises one or more of N, NR, O, or S;
A¹ and A³ are each independently a 3- to 7-membered carbocyclic or heterocyclic ring; and
B¹ and B² are each independently a 5- or 6-membered aromatic or heteroaromatic ring;
wherein R, R′, R″, and R′″ are each independently H, C₁₋₆ alkyl, or C₁₋₆ alkene; and
wherein the R, R′, R″, R′″, A¹, A³, B¹, B², and the fused bicyclic aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

4. The compound of claim 1, or a stereoisomer or a pharmaceutically acceptable salt thereof, wherein the compound is of Formula (IVa) or (IVb): 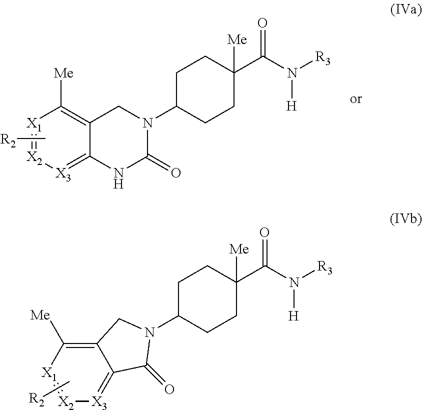
wherein:
X₁, X₂ and X₃ are independently CH, C, or N
R₂ is absent, C₁-C₃ alkyl, C₁-C₃ alkene, NR′R″, NHR′NR″R′″, R′NR″R″′, NHC(O)R′NR″R″′, OR, CN, NO₂, C(O)R, R′OR″, R′NR″C(O)R′″, C(O)OR, C(O)NR′R″, C(O)NHR′NR″R′″, P(O)R′R″, R′NR″S(O)₂R′″, —O—R′NR″R′″, halo, CF₃, A¹, B¹, —ORA¹, —ORB¹, —OA¹, —OB¹, ROA¹, ROB¹, NR′R″A¹, NR′R″B¹, N(R)B¹, N(R)A¹, NR′C(O)R″A¹, NR′C(O)R″B¹, N(R)C(O)A¹, N(R)C(O)B¹, RA¹, RB¹:
R₃ is A³, B², or a fused bicyclic aromatic ring system, wherein the fused bicyclic aromatic ring system optionally comprises one or more of N, NR, O, or S;
A¹ and A³ are each independently a 3- to 7-membered carbocyclic or heterocyclic ring; and
B¹ and B² are each independently a 5- or 6-membered aromatic or heteroaromatic ring;
wherein R, R′, R″, and R′″ are each independently H, C₁₋₆ alkyl, or C₁₋₆ alkene; and
wherein the R, R′, R″, R′″, A¹, A³, B¹, B², and the fused bicyclic aromatic ring system is unsubstituted or substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring.

5. The compound of claim 1, or a stereoisomer or a pharmaceutically acceptable salt thereof, wherein the compound of Formula (IIIa) or Formula (IIIb) comprises:

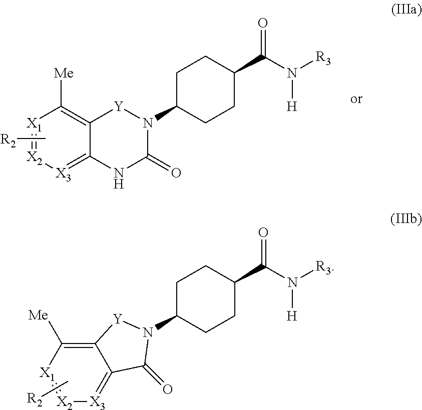

6. The compound of claim 1, or a stereoisomer or a pharmaceutically acceptable salt thereof, wherein the compound of Formula (IVa) or Formula (IVb) comprises:

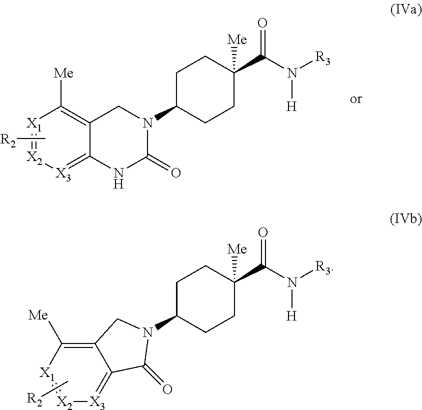

7. The compound of claim 1, or a stereoisomer, or a pharmaceutically acceptable salt thereof, wherein at least two of X₁, X₂, and X₃ are CH or N;
optionally, R₁ is H, C₁₋₆ alkyl, or halo;
optionally, R₂ is absent, NR′R″, OR, CN, a 5- or 6-membered heteroaromatic ring.

8. (canceled)

9. (canceled)

10. The compound of claim 7, or a stereoisomer, or a pharmaceutically acceptable salt thereof, wherein R₂ is attached to X₂ or X₃.

11. The compound of claim 1, or a stereoisomer, or a pharmaceutically acceptable salt thereof, wherein the A¹ or B¹ of R₂ is 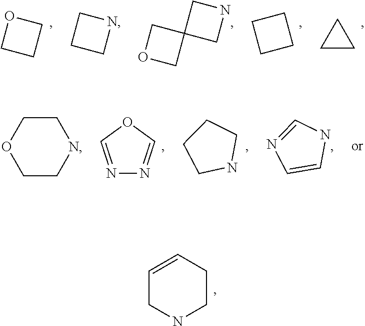
optionally substituted with one or two substituents selected from the group consisting of C₁₋₆ alkyl, —O—C₁₋₆ alkyl, CN, OH, halo, and NH₂;
optionally, Y is CR′R″ or CH₂;
optionally, Q is

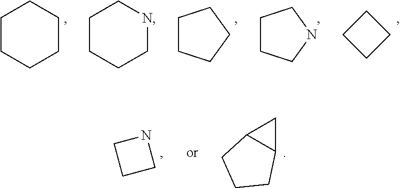

12. (canceled)

13. The compound of claim 3, or a stereoisomer, or a pharmaceutically acceptable salt thereof, wherein in Formula (IIIa) the Y is NR, or wherein in Formula (IIIb) the Y is NR, O, or S.

14. (canceled)

15. The compound of claim 2, or a stereoisomer, or a pharmaceutically acceptable salt thereof, wherein R₄ is a C₁₋₃ alkyl.

16. The compound of claim 1, or a stereoisomer, or a pharmaceutically acceptable salt thereof, wherein Z is a single bond;
optionally, the 3- to 7-membered carbocyclic or heterocyclic ring comprises a spirocyclic ring;
optionally, the A³ or B² of R₃ is substituted with one or more substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring;
optionally, A² is substituted with one or two substituents selected form the group consisting of CN, CF₃, OR, ═O, ROH, NR′R″, C₁₋₆ alkyl, —O—C₁₋₆ alkyl, C₁₋₆ alkene, F, Cl, Br, I, and a 3- to 7-membered carbocyclic or heterocyclic ring.

17. (canceled)

18. (canceled)

19. The compound of claim 16, or a stereoisomer, or a pharmaceutically acceptable salt thereof, wherein R₃ is the 5- or 6-membered aromatic or heteroaromatic ring or fused bicyclic aromatic ring system substituted with one or two substituents selected form the group consisting of CN, CF₃, OR, ═O, ROH, NR′R″, C₁₋₆ alkyl, —O—C₁₋₆ alkyl, C₁₋₆ alkene, halo, and a 3- to 7-membered carbocyclic or heterocyclic ring;
optionally, R₃ is 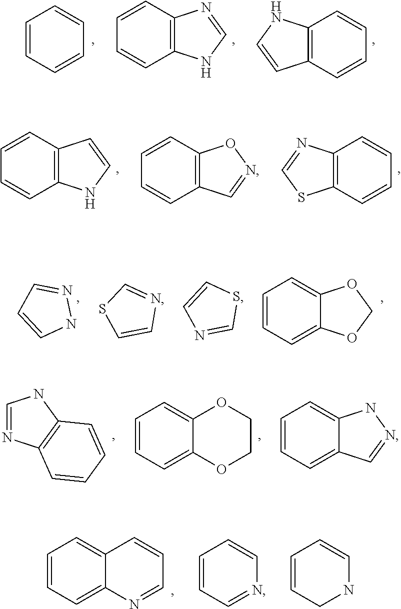
and is unsubstituted or substituted with one, two, or three substituents selected from the group consisting of CN, CF₃, OR, ═O, C₁₋₆ alkyl, —O—C₁₋₆ alkyl, C₁₋₆ alkene, ROH, halo, and a 3- to 7-membered carbocyclic or heterocyclic ring.

20. (canceled)

21. (canceled)

22. The compound of claim 1, or a stereoisomer, or a pharmaceutically acceptable salt thereof, selected from the group consisting of

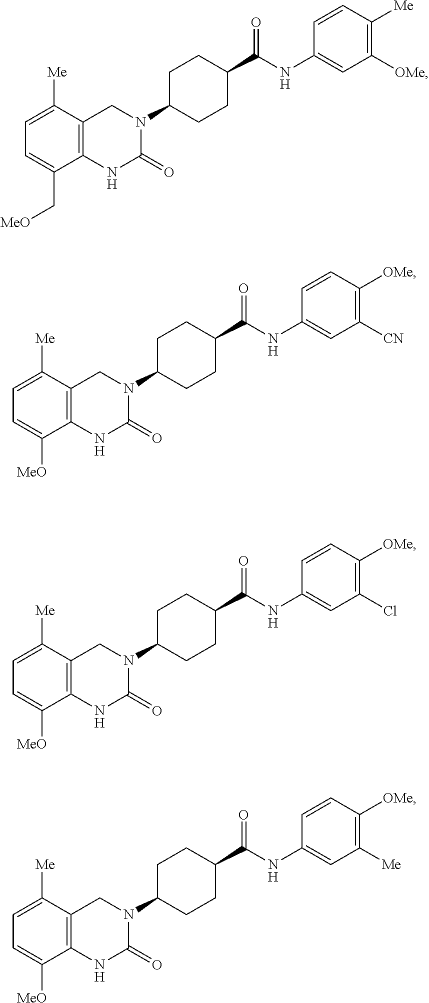 [SMILES: COCc1ccc(C)c2c1NC(=O)N([C@H]1CC[C@@H](C(=O)Nc3ccc(C)c(OC)c3)CC1)C2 ; COc1ccc(NC(=O)[C@H]2CC[C@@H](N3Cc4c(C)ccc(OC)c4NC3=O)CC2)cc1C#N ; COc1ccc(NC(=O)[C@H]2CC[C@@H](N3Cc4c(C)ccc(OC)c4NC3=O)CC2)cc1Cl ; COc1ccc(NC(=O)[C@H]2CC[C@@H](N3Cc4c(C)ccc(OC)c4NC3=O)CC2)cc1C]

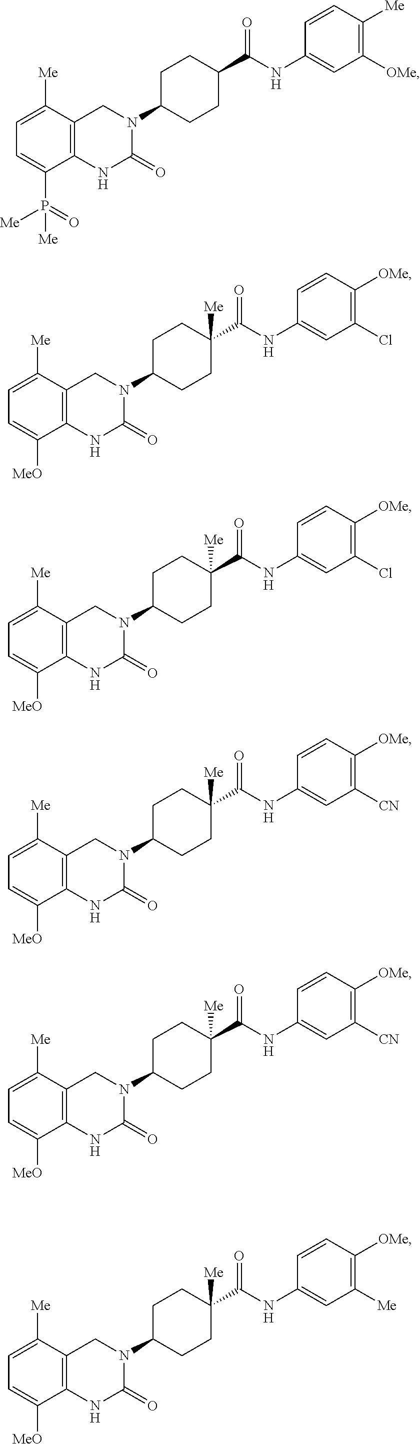 [SMILES: COc1cc(NC(=O)[C@H]2CC[C@@H](N3Cc4c(C)ccc(P(C)(C)=O)c4NC3=O)CC2)ccc1C ; COc1ccc(NC(=O)[C@]2(C)CC[C@@H](N3Cc4c(C)ccc(OC)c4NC3=O)CC2)cc1Cl ; COc1ccc(NC(=O)[C@]2(C)CC[C@H](N3Cc4c(C)ccc(OC)c4NC3=O)CC2)cc1Cl ; COc1ccc(NC(=O)[C@]2(C)CC[C@@H](N3Cc4c(C)ccc(OC)c4NC3=O)CC2)cc1C#N ; COc1ccc(NC(=O)[C@]2(C)CC[C@H](N3Cc4c(C)ccc(OC)c4NC3=O)CC2)cc1C#N ; COc1ccc(NC(=O)[C@]2(C)CC[C@@H](N3Cc4c(C)ccc(OC)c4NC3=O)CC2)cc1C]

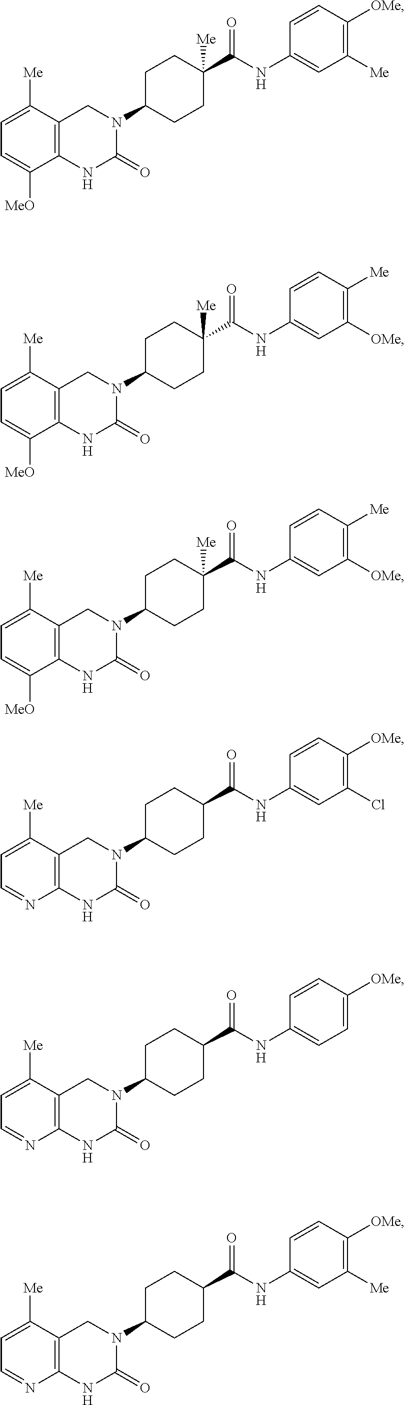 [SMILES: COc1ccc(NC(=O)[C@]2(C)CC[C@H](N3Cc4c(C)ccc(OC)c4NC3=O)CC2)cc1C ; COc1cc(NC(=O)[C@]2(C)CC[C@@H](N3Cc4c(C)ccc(OC)c4NC3=O)CC2)ccc1C ; COc1cc(NC(=O)[C@]2(C)CC[C@H](N3Cc4c(C)ccc(OC)c4NC3=O)CC2)ccc1C ; COc1ccc(NC(=O)[C@H]2CC[C@@H](N3Cc4c(C)ccnc4NC3=O)CC2)cc1Cl ; COc1ccc(NC(=O)[C@H]2CC[C@@H](N3Cc4c(C)ccnc4NC3=O)CC2)cc1 ; COc1ccc(NC(=O)[C@H]2CC[C@@H](N3Cc4c(C)ccnc4NC3=O)CC2)cc1C]

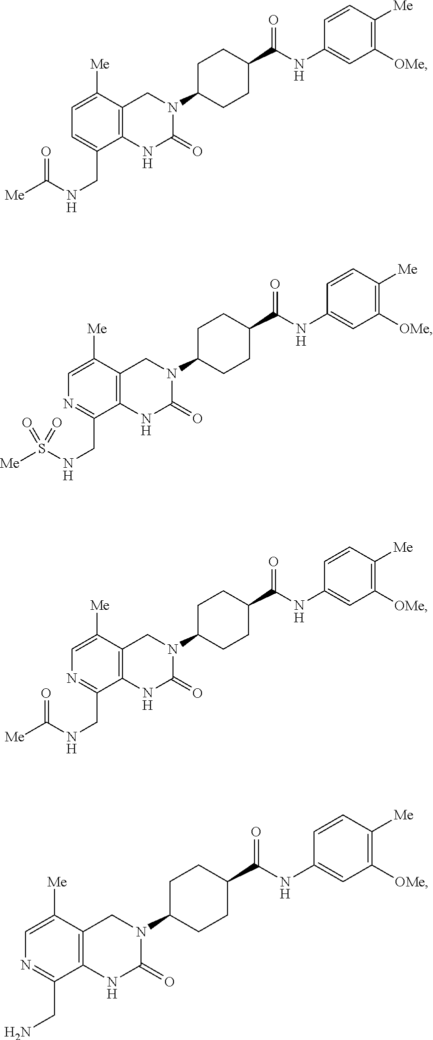 [SMILES: COc1cc(NC(=O)[C@H]2CC[C@@H](N3Cc4c(C)ccc(CNC(C)=O)c4NC3=O)CC2)ccc1C ; COc1cc(NC(=O)[C@H]2CC[C@@H](N3Cc4c(C)cnc(CNS(C)(=O)=O)c4NC3=O)CC2)ccc1C ; COc1cc(NC(=O)[C@H]2CC[C@@H](N3Cc4c(C)cnc(CNC(C)=O)c4NC3=O)CC2)ccc1C ; COc1cc(NC(=O)[C@H]2CC[C@@H](N3Cc4c(C)cnc(CN)c4NC3=O)CC2)ccc1C]

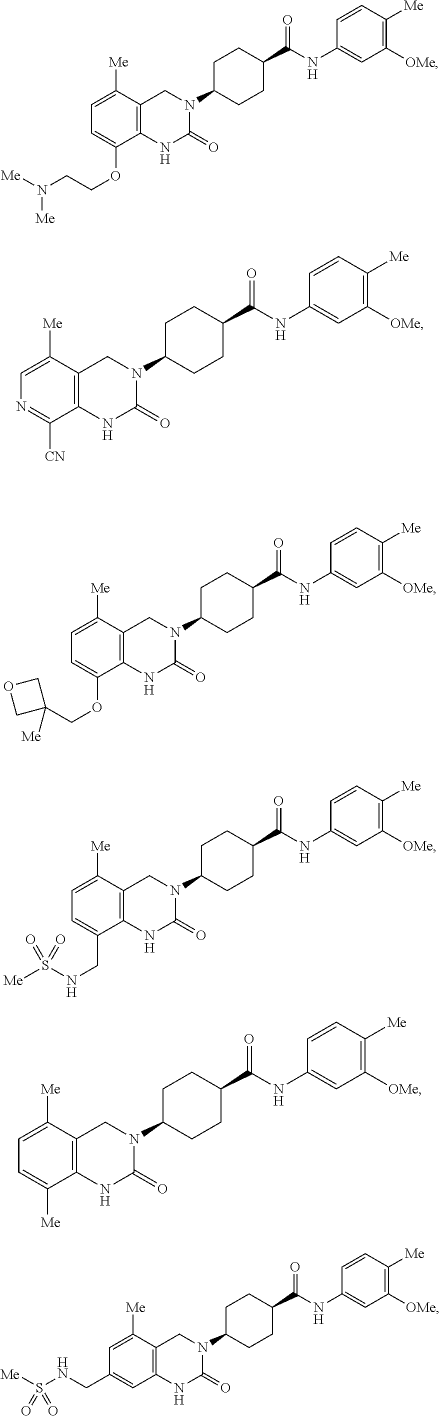 [SMILES: COc1cc(NC(=O)[C@H]2CC[C@@H](N3Cc4c(C)ccc(OCCN(C)C)c4NC3=O)CC2)ccc1C ; COc1cc(NC(=O)[C@H]2CC[C@@H](N3Cc4c(C)cnc(C#N)c4NC3=O)CC2)ccc1C ; COc1cc(NC(=O)[C@H]2CC[C@@H](N3Cc4c(C)ccc(OCC5(C)COC5)c4NC3=O)CC2)ccc1C ; COc1cc(NC(=O)[C@H]2CC[C@@H](N3Cc4c(C)ccc(CNS(C)(=O)=O)c4NC3=O)CC2)ccc1C ; COc1cc(NC(=O)[C@H]2CC[C@@H](N3Cc4c(C)ccc(C)c4NC3=O)CC2)ccc1C ; COc1cc(NC(=O)[C@H]2CC[C@@H](N3Cc4c(C)cc(CNS(C)(=O)=O)cc4NC3=O)CC2)ccc1C]

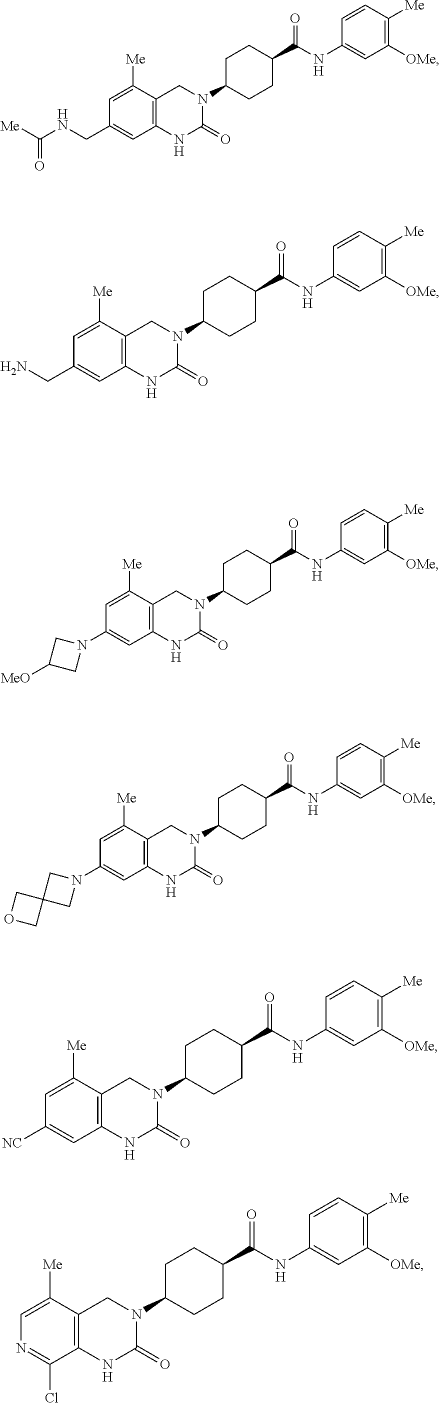 [SMILES: COc1cc(NC(=O)[C@H]2CC[C@@H](N3Cc4c(C)cc(CNC(C)=O)cc4NC3=O)CC2)ccc1C ; COc1cc(NC(=O)[C@H]2CC[C@@H](N3Cc4c(C)cc(CN)cc4NC3=O)CC2)ccc1C ; COc1cc(NC(=O)[C@H]2CC[C@@H](N3Cc4c(C)cc(N5CC(OC)C5)cc4NC3=O)CC2)ccc1C ; COc1cc(NC(=O)[C@H]2CC[C@@H](N3Cc4c(C)cc(N5CC6(COC6)C5)cc4NC3=O)CC2)ccc1C ; COc1cc(NC(=O)[C@H]2CC[C@@H](N3Cc4c(C)cc(C#N)cc4NC3=O)CC2)ccc1C ; COc1cc(NC(=O)[C@H]2CC[C@@H](N3Cc4c(C)cnc(Cl)c4NC3=O)CC2)ccc1C]

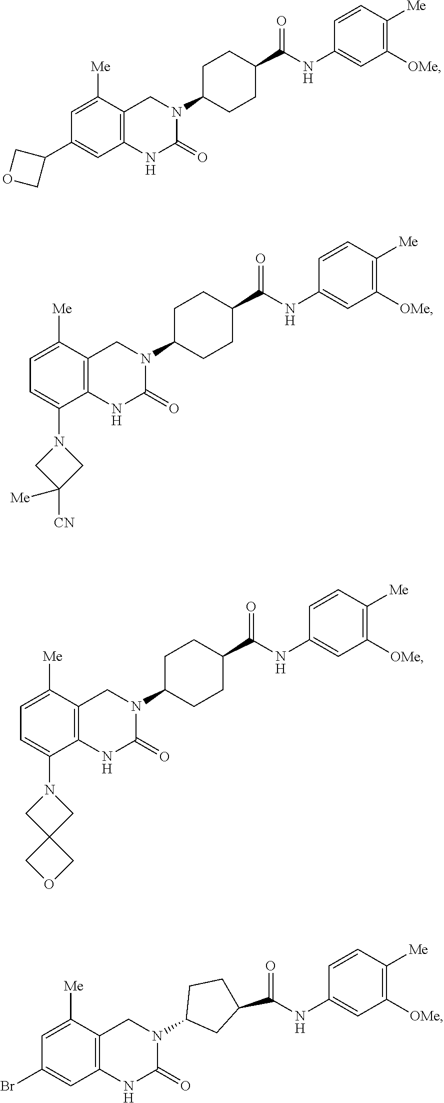 [SMILES: COc1cc(NC(=O)[C@H]2CC[C@@H](N3Cc4c(C)cc(C5COC5)cc4NC3=O)CC2)ccc1C ; COc1cc(NC(=O)[C@H]2CC[C@@H](N3Cc4c(C)ccc(N5CC(C)(C#N)C5)c4NC3=O)CC2)ccc1C ; COc1cc(NC(=O)[C@H]2CC[C@@H](N3Cc4c(C)ccc(N5CC6(COC6)C5)c4NC3=O)CC2)ccc1C ; COc1cc(NC(=O)[C@@H]2CC[C@@H](N3Cc4c(C)cc(Br)cc4NC3=O)C2)ccc1C]

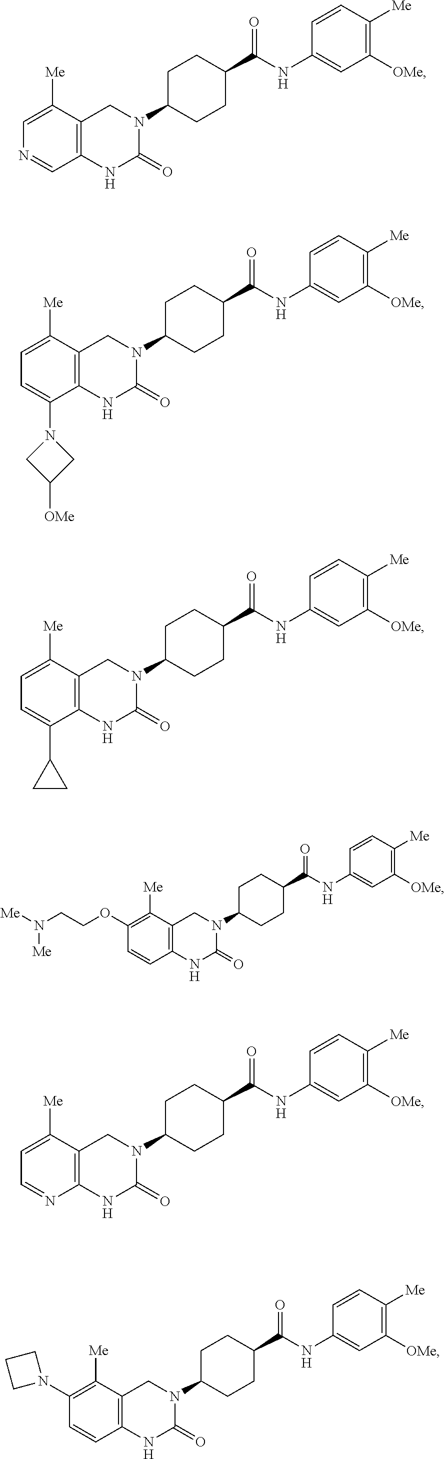 [SMILES: COc1cc(NC(=O)[C@H]2CC[C@@H](N3Cc4c(C)cncc4NC3=O)CC2)ccc1C ; COc1cc(NC(=O)[C@H]2CC[C@@H](N3Cc4c(C)ccc(N5CC(OC)C5)c4NC3=O)CC2)ccc1C ; COc1cc(NC(=O)[C@H]2CC[C@@H](N3Cc4c(C)ccc(C5CC5)c4NC3=O)CC2)ccc1C ; COc1cc(NC(=O)[C@H]2CC[C@@H](N3Cc4c(ccc(OCCN(C)C)c4C)NC3=O)CC2)ccc1C ; COc1cc(NC(=O)[C@H]2CC[C@@H](N3Cc4c(C)ccnc4NC3=O)CC2)ccc1C ; COc1cc(NC(=O)[C@H]2CC[C@@H](N3Cc4c(ccc(N5CCC5)c4C)NC3=O)CC2)ccc1C]

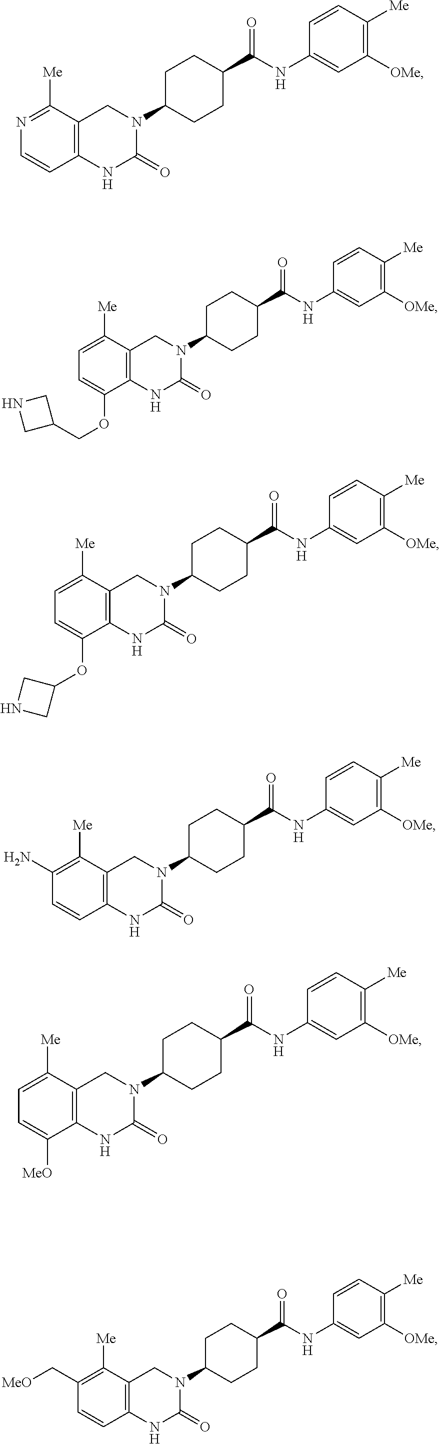 [SMILES: COc1cc(NC(=O)[C@H]2CC[C@@H](N3Cc4c(ccnc4C)NC3=O)CC2)ccc1C ; COc1cc(NC(=O)[C@H]2CC[C@@H](N3Cc4c(C)ccc(OCC5CNC5)c4NC3=O)CC2)ccc1C ; COc1cc(NC(=O)[C@H]2CC[C@@H](N3Cc4c(C)ccc(OC5CNC5)c4NC3=O)CC2)ccc1C ; COc1cc(NC(=O)[C@H]2CC[C@@H](N3Cc4c(ccc(N)c4C)NC3=O)CC2)ccc1C ; COc1cc(NC(=O)[C@H]2CC[C@@H](N3Cc4c(C)ccc(OC)c4NC3=O)CC2)ccc1C ; COCc1ccc2c(c1C)CN([C@H]1CC[C@@H](C(=O)Nc3ccc(C)c(OC)c3)CC1)C(=O)N2]

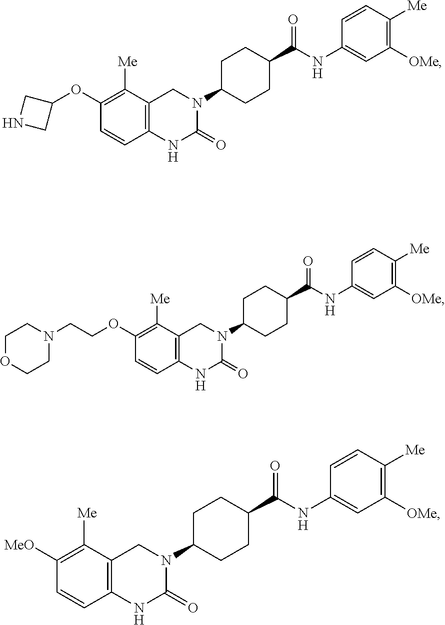 [SMILES: COc1cc(NC(=O)[C@H]2CC[C@@H](N3Cc4c(ccc(OC5CNC5)c4C)NC3=O)CC2)ccc1C ; COc1cc(NC(=O)[C@H]2CC[C@@H](N3Cc4c(ccc(OCCN5CCOCC5)c4C)NC3=O)CC2)ccc1C ; COc1cc(NC(=O)[C@H]2CC[C@@H](N3Cc4c(ccc(OC)c4C)NC3=O)CC2)ccc1C]

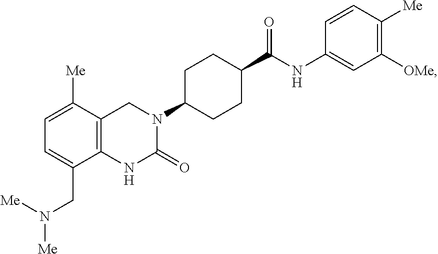 [SMILES: COc1cc(NC(=O)[C@H]2CC[C@@H](N3Cc4c(C)ccc(CN(C)C)c4NC3=O)CC2)ccc1C]

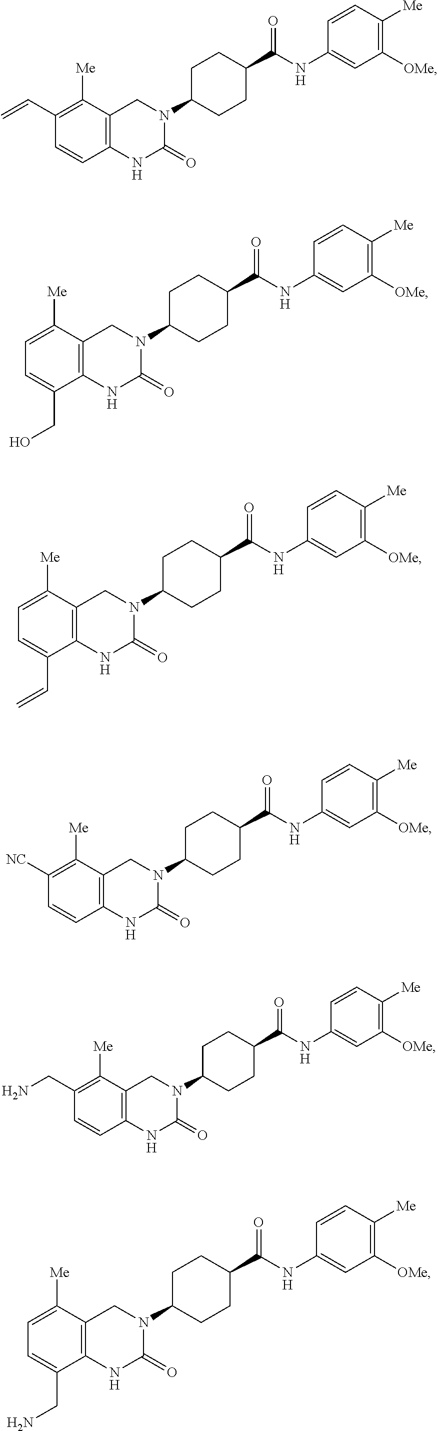 [SMILES: C=Cc1ccc2c(c1C)CN([C@H]1CC[C@@H](C(=O)Nc3ccc(C)c(OC)c3)CC1)C(=O)N2 ; COc1cc(NC(=O)[C@H]2CC[C@@H](N3Cc4c(C)ccc(CO)c4NC3=O)CC2)ccc1C ; C=Cc1ccc(C)c2c1NC(=O)N([C@H]1CC[C@@H](C(=O)Nc3ccc(C)c(OC)c3)CC1)C2 ; COc1cc(NC(=O)[C@H]2CC[C@@H](N3Cc4c(ccc(C#N)c4C)NC3=O)CC2)ccc1C ; COc1cc(NC(=O)[C@H]2CC[C@@H](N3Cc4c(ccc(CN)c4C)NC3=O)CC2)ccc1C ; COc1cc(NC(=O)[C@H]2CC[C@@H](N3Cc4c(C)ccc(CN)c4NC3=O)CC2)ccc1C]

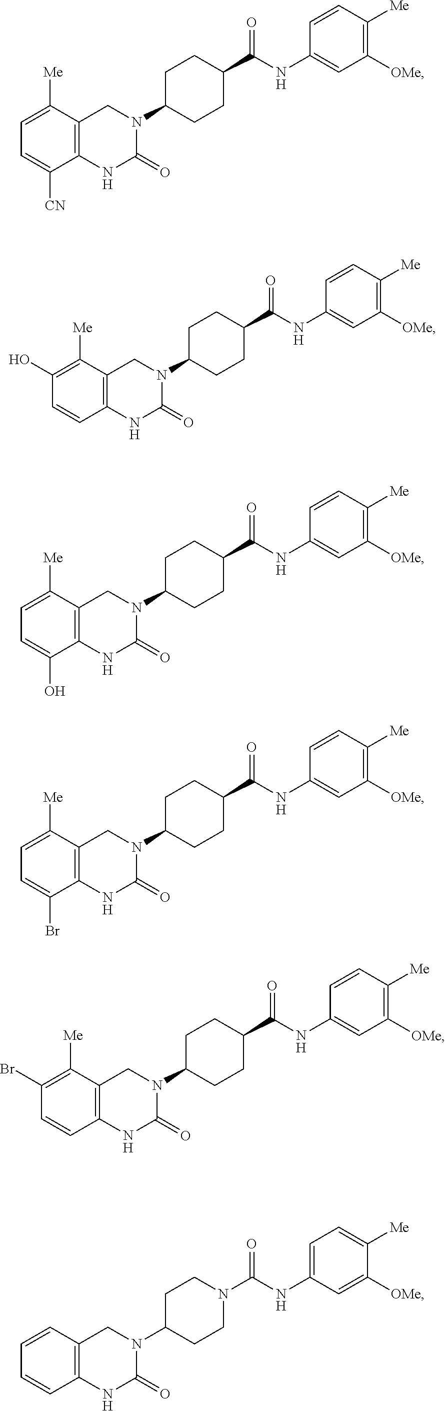 [SMILES: COc1cc(NC(=O)[C@H]2CC[C@@H](N3Cc4c(C)ccc(C#N)c4NC3=O)CC2)ccc1C ; COc1cc(NC(=O)[C@H]2CC[C@@H](N3Cc4c(ccc(O)c4C)NC3=O)CC2)ccc1C ; COc1cc(NC(=O)[C@H]2CC[C@@H](N3Cc4c(C)ccc(O)c4NC3=O)CC2)ccc1C ; COc1cc(NC(=O)[C@H]2CC[C@@H](N3Cc4c(C)ccc(Br)c4NC3=O)CC2)ccc1C ; COc1cc(NC(=O)[C@H]2CC[C@@H](N3Cc4c(ccc(Br)c4C)NC3=O)CC2)ccc1C ; COc1cc(NC(=O)N2CCC(N3Cc4ccccc4NC3=O)CC2)ccc1C]

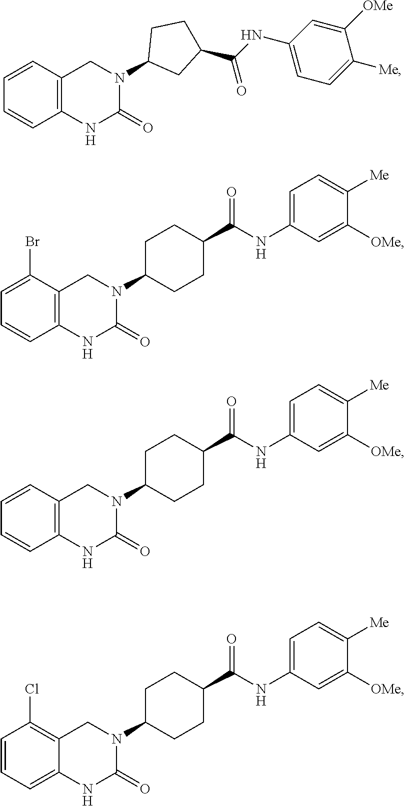 [SMILES: COc1cc(NC(=O)[C@@H]2CC[C@H](N3Cc4ccccc4NC3=O)C2)ccc1C ; COc1cc(NC(=O)[C@H]2CC[C@@H](N3Cc4c(Br)cccc4NC3=O)CC2)ccc1C ; COc1cc(NC(=O)[C@H]2CC[C@@H](N3Cc4ccccc4NC3=O)CC2)ccc1C ; COc1cc(NC(=O)[C@H]2CC[C@@H](N3Cc4c(Cl)cccc4NC3=O)CC2)ccc1C]

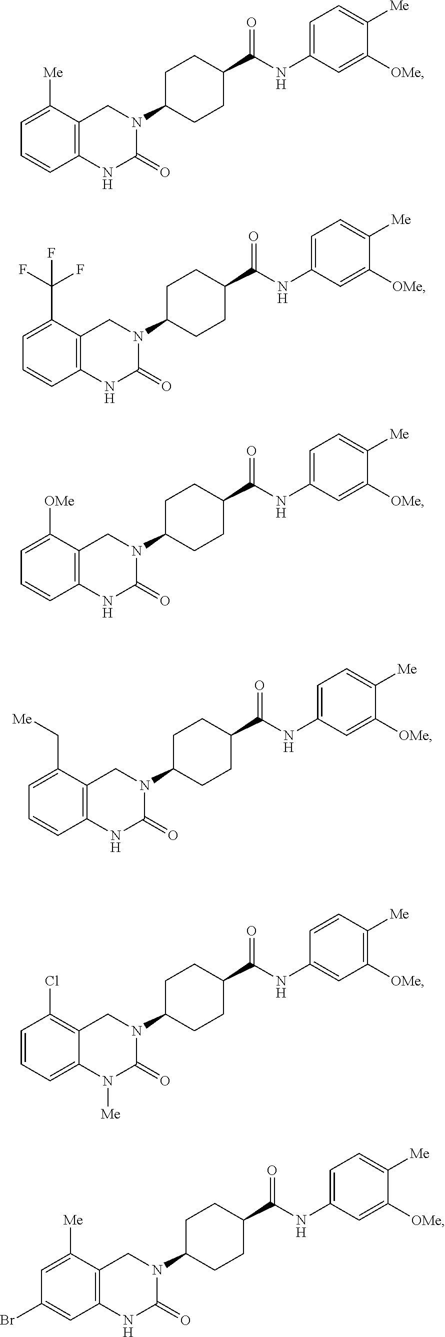 [SMILES: COc1cc(NC(=O)[C@H]2CC[C@@H](N3Cc4c(C)cccc4NC3=O)CC2)ccc1C ; COc1cc(NC(=O)[C@H]2CC[C@@H](N3Cc4c(cccc4C(F)(F)F)NC3=O)CC2)ccc1C ; COc1cc(NC(=O)[C@H]2CC[C@@H](N3Cc4c(cccc4OC)NC3=O)CC2)ccc1C ; CCc1cccc2c1CN([C@H]1CC[C@@H](C(=O)Nc3ccc(C)c(OC)c3)CC1)C(=O)N2 ; COc1cc(NC(=O)[C@H]2CC[C@@H](N3Cc4c(Cl)cccc4N(C)C3=O)CC2)ccc1C ; COc1cc(NC(=O)[C@H]2CC[C@@H](N3Cc4c(C)cc(Br)cc4NC3=O)CC2)ccc1C]

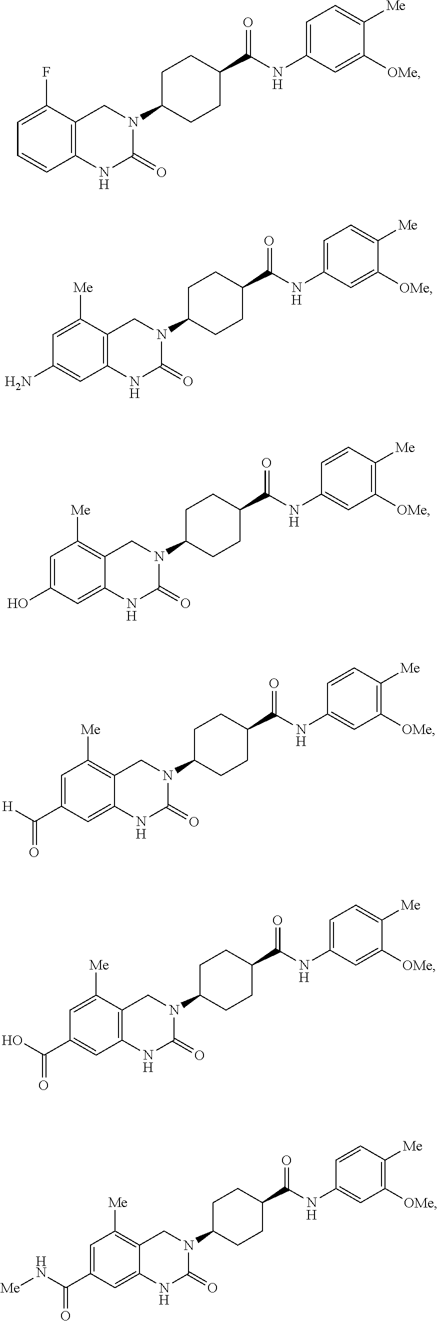 [SMILES: COc1cc(NC(=O)[C@H]2CC[C@@H](N3Cc4c(F)cccc4NC3=O)CC2)ccc1C ; COc1cc(NC(=O)[C@H]2CC[C@@H](N3Cc4c(C)cc(N)cc4NC3=O)CC2)ccc1C ; COc1cc(NC(=O)[C@H]2CC[C@@H](N3Cc4c(C)cc(O)cc4NC3=O)CC2)ccc1C ; COc1cc(NC(=O)[C@H]2CC[C@@H](N3Cc4c(C)cc(C=O)cc4NC3=O)CC2)ccc1C ; COc1cc(NC(=O)[C@H]2CC[C@@H](N3Cc4c(C)cc(C(=O)O)cc4NC3=O)CC2)ccc1C ; CNC(=O)c1cc(C)c2c(c1)NC(=O)N([C@H]1CC[C@@H](C(=O)Nc3ccc(C)c(OC)c3)CC1)C2]

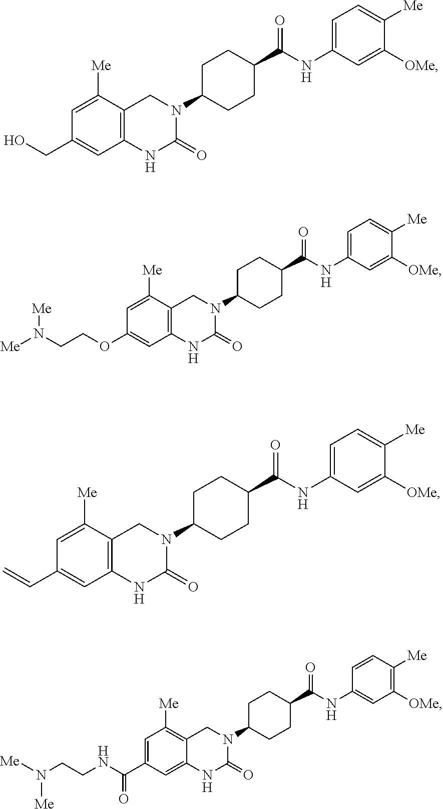 [SMILES: COc1cc(NC(=O)[C@H]2CC[C@@H](N3Cc4c(C)cc(CO)cc4NC3=O)CC2)ccc1C ; COc1cc(NC(=O)[C@H]2CC[C@@H](N3Cc4c(C)cc(OCCN(C)C)cc4NC3=O)CC2)ccc1C ; C=Cc1cc(C)c2c(c1)NC(=O)N([C@H]1CC[C@@H](C(=O)Nc3ccc(C)c(OC)c3)CC1)C2 ; COc1cc(NC(=O)[C@H]2CC[C@@H](N3Cc4c(C)cc(C(=O)NCCN(C)C)cc4NC3=O)CC2)ccc1C]

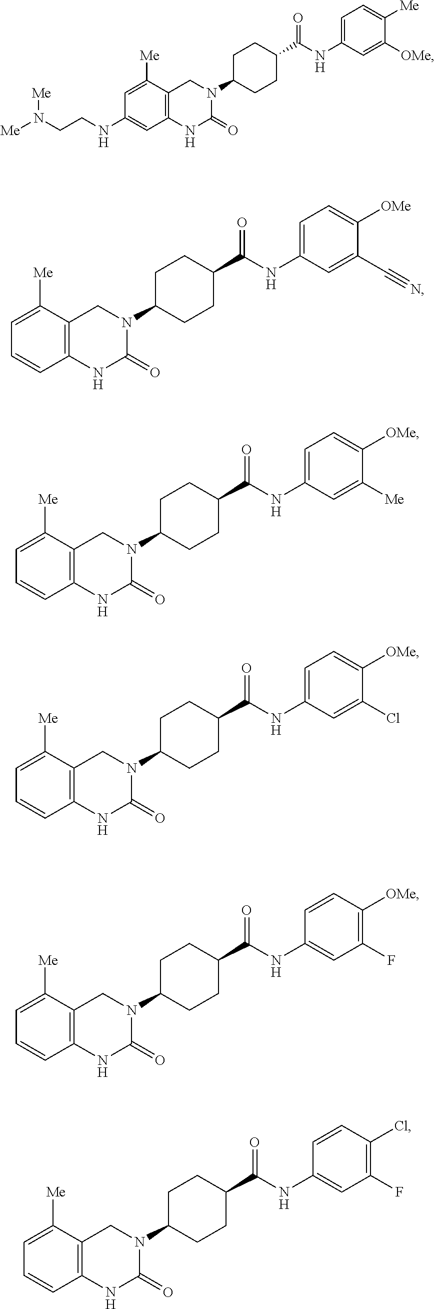 [SMILES: COc1cc(NC(=O)[C@H]2CC[C@H](N3Cc4c(C)cc(NCCN(C)C)cc4NC3=O)CC2)ccc1C ; COc1ccc(NC(=O)[C@H]2CC[C@@H](N3Cc4c(C)cccc4NC3=O)CC2)cc1C#N ; COc1ccc(NC(=O)[C@H]2CC[C@@H](N3Cc4c(C)cccc4NC3=O)CC2)cc1C ; COc1ccc(NC(=O)[C@H]2CC[C@@H](N3Cc4c(C)cccc4NC3=O)CC2)cc1Cl ; COc1ccc(NC(=O)[C@H]2CC[C@@H](N3Cc4c(C)cccc4NC3=O)CC2)cc1F ; Cc1cccc2c1CN([C@H]1CC[C@@H](C(=O)Nc3ccc(Cl)c(F)c3)CC1)C(=O)N2]

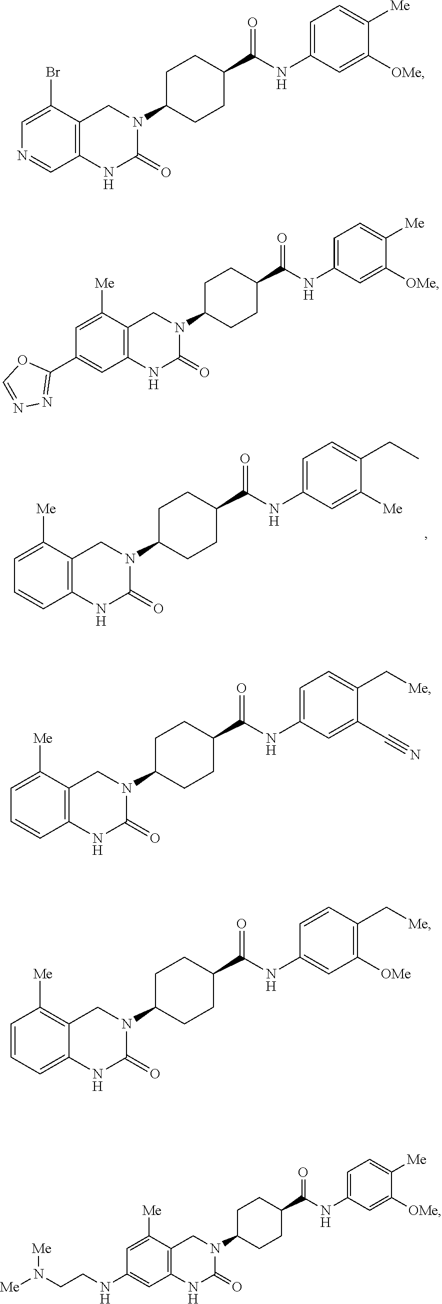 [SMILES: COc1cc(NC(=O)[C@H]2CC[C@@H](N3Cc4c(Br)cncc4NC3=O)CC2)ccc1C ; COc1cc(NC(=O)[C@H]2CC[C@@H](N3Cc4c(C)cc(-c5nnco5)cc4NC3=O)CC2)ccc1C ; CCc1ccc(NC(=O)[C@H]2CC[C@@H](N3Cc4c(C)cccc4NC3=O)CC2)cc1C ; CCc1ccc(NC(=O)[C@H]2CC[C@@H](N3Cc4c(C)cccc4NC3=O)CC2)cc1C#N ; CCc1ccc(NC(=O)[C@H]2CC[C@@H](N3Cc4c(C)cccc4NC3=O)CC2)cc1OC ; COc1cc(NC(=O)[C@H]2CC[C@@H](N3Cc4c(C)cc(NCCN(C)C)cc4NC3=O)CC2)ccc1C]

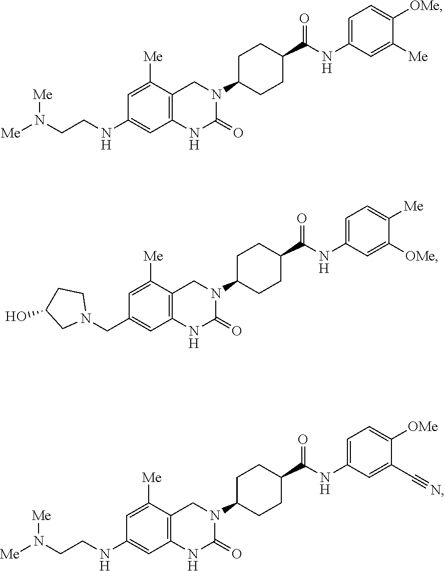 [SMILES: COc1ccc(NC(=O)[C@H]2CC[C@@H](N3Cc4c(C)cc(NCCN(C)C)cc4NC3=O)CC2)cc1C ; COc1cc(NC(=O)[C@H]2CC[C@@H](N3Cc4c(C)cc(CN5CC[C@@H](O)C5)cc4NC3=O)CC2)ccc1C ; COc1ccc(NC(=O)[C@H]2CC[C@@H](N3Cc4c(C)cc(NCCN(C)C)cc4NC3=O)CC2)cc1C#N]

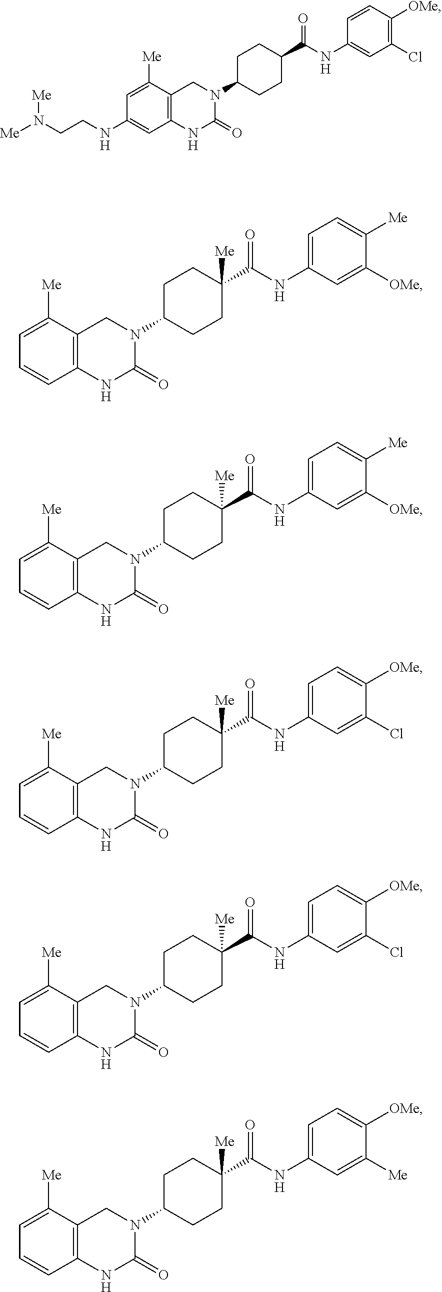 [SMILES: COc1ccc(NC(=O)[C@H]2CC[C@@H](N3Cc4c(C)cc(NCCN(C)C)cc4NC3=O)CC2)cc1Cl ; COc1cc(NC(=O)[C@]2(C)CC[C@H](N3Cc4c(C)cccc4NC3=O)CC2)ccc1C ; COc1cc(NC(=O)[C@]2(C)CC[C@@H](N3Cc4c(C)cccc4NC3=O)CC2)ccc1C ; COc1ccc(NC(=O)[C@]2(C)CC[C@H](N3Cc4c(C)cccc4NC3=O)CC2)cc1Cl ; COc1ccc(NC(=O)[C@]2(C)CC[C@@H](N3Cc4c(C)cccc4NC3=O)CC2)cc1Cl ; COc1ccc(NC(=O)[C@]2(C)CC[C@H](N3Cc4c(C)cccc4NC3=O)CC2)cc1C]

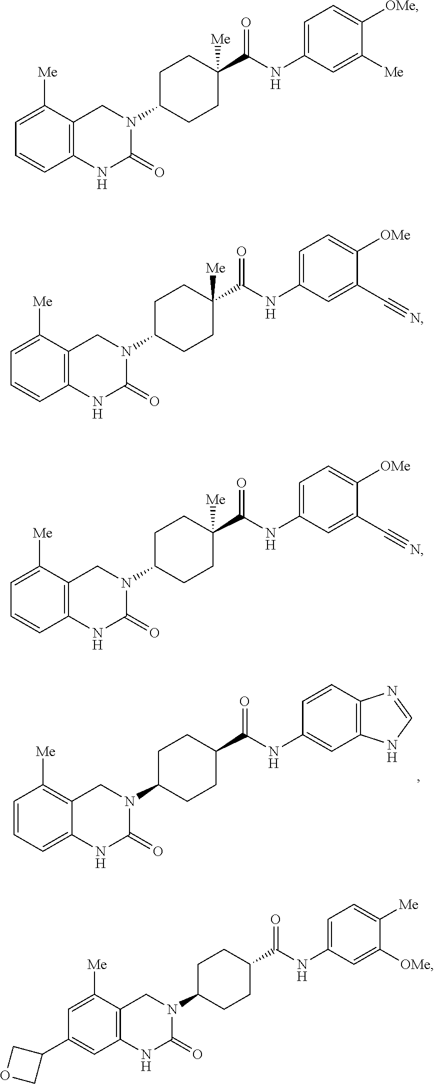 [SMILES: COc1ccc(NC(=O)[C@]2(C)CC[C@@H](N3Cc4c(C)cccc4NC3=O)CC2)cc1C ; COc1ccc(NC(=O)[C@]2(C)CC[C@H](N3Cc4c(C)cccc4NC3=O)CC2)cc1C#N ; COc1ccc(NC(=O)[C@]2(C)CC[C@@H](N3Cc4c(C)cccc4NC3=O)CC2)cc1C#N ; Cc1cccc2c1CN([C@H]1CC[C@@H](C(=O)Nc3ccc4nc[nH]c4c3)CC1)C(=O)N2 ; COc1cc(NC(=O)[C@H]2CC[C@H](N3Cc4c(C)cc(C5COC5)cc4NC3=O)CC2)ccc1C]

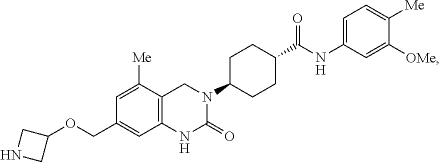 [SMILES: COc1cc(NC(=O)[C@H]2CC[C@H](N3Cc4c(C)cc(COC5CNC5)cc4NC3=O)CC2)ccc1C]

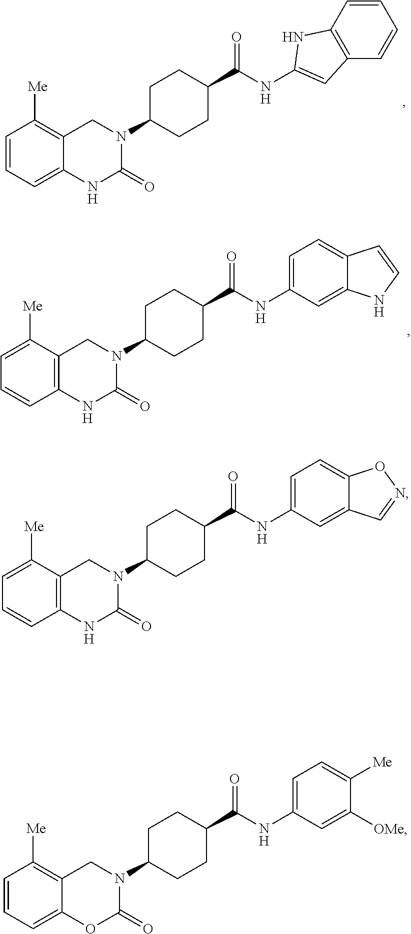 [SMILES: Cc1cccc2c1CN([C@H]1CC[C@@H](C(=O)Nc3cc4ccccc4[nH]3)CC1)C(=O)N2 ; Cc1cccc2c1CN([C@H]1CC[C@@H](C(=O)Nc3ccc4cc[nH]c4c3)CC1)C(=O)N2 ; Cc1cccc2c1CN([C@H]1CC[C@@H](C(=O)Nc3ccc4oncc4c3)CC1)C(=O)N2 ; COc1cc(NC(=O)[C@H]2CC[C@@H](N3Cc4c(C)cccc4OC3=O)CC2)ccc1C]

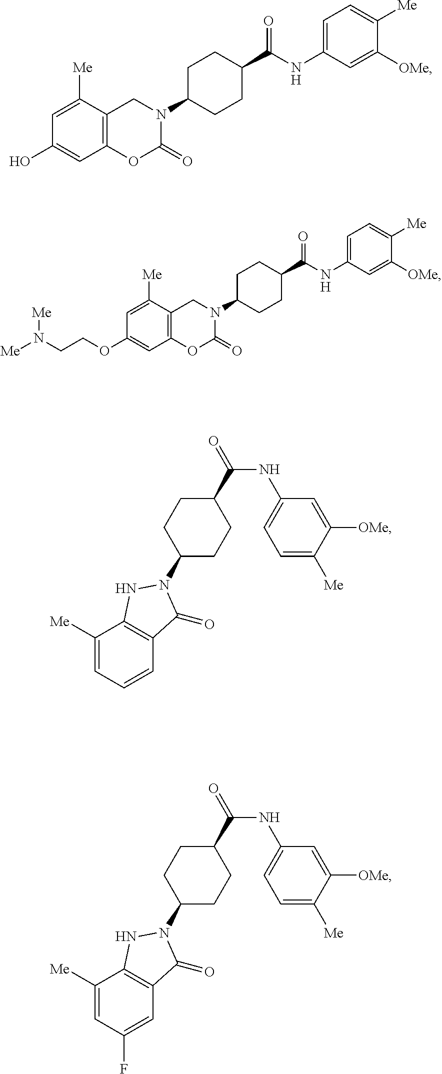 [SMILES: COc1cc(NC(=O)[C@H]2CC[C@@H](N3Cc4c(C)cc(O)cc4OC3=O)CC2)ccc1C ; COc1cc(NC(=O)[C@H]2CC[C@@H](N3Cc4c(C)cc(OCCN(C)C)cc4OC3=O)CC2)ccc1C ; COc1cc(NC(=O)[C@H]2CC[C@@H](n3[nH]c4c(C)cccc4c3=O)CC2)ccc1C ; COc1cc(NC(=O)[C@H]2CC[C@@H](n3[nH]c4c(C)cc(F)cc4c3=O)CC2)ccc1C]

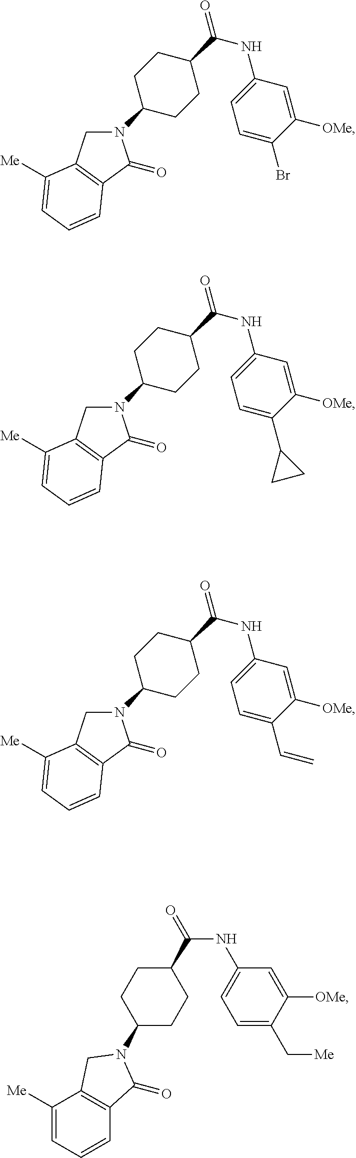 [SMILES: COc1cc(NC(=O)[C@H]2CC[C@@H](N3Cc4c(C)cccc4C3=O)CC2)ccc1Br ; COc1cc(NC(=O)[C@H]2CC[C@@H](N3Cc4c(C)cccc4C3=O)CC2)ccc1C1CC1 ; C=Cc1ccc(NC(=O)[C@H]2CC[C@@H](N3Cc4c(C)cccc4C3=O)CC2)cc1OC ; CCc1ccc(NC(=O)[C@H]2CC[C@@H](N3Cc4c(C)cccc4C3=O)CC2)cc1OC]

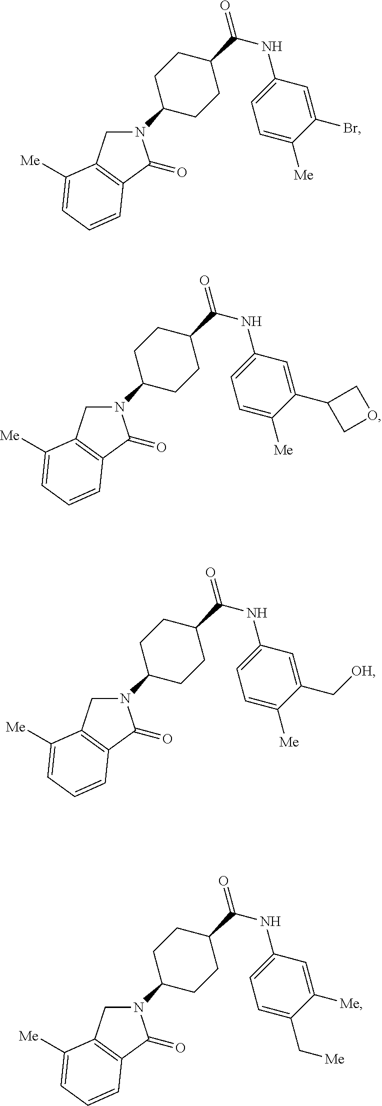 [SMILES: Cc1ccc(NC(=O)[C@H]2CC[C@@H](N3Cc4c(C)cccc4C3=O)CC2)cc1Br ; Cc1ccc(NC(=O)[C@H]2CC[C@@H](N3Cc4c(C)cccc4C3=O)CC2)cc1C1COC1 ; Cc1ccc(NC(=O)[C@H]2CC[C@@H](N3Cc4c(C)cccc4C3=O)CC2)cc1CO ; CCc1ccc(NC(=O)[C@H]2CC[C@@H](N3Cc4c(C)cccc4C3=O)CC2)cc1C]

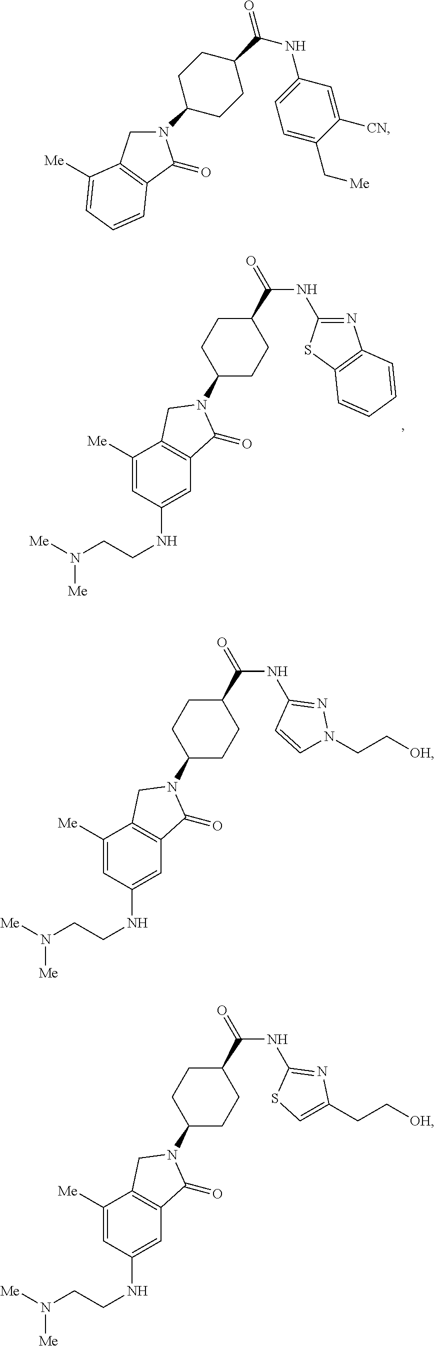 [SMILES: CCc1ccc(NC(=O)[C@H]2CC[C@@H](N3Cc4c(C)cccc4C3=O)CC2)cc1C#N ; Cc1cc(NCCN(C)C)cc2c1CN([C@H]1CC[C@@H](C(=O)Nc3nc4ccccc4s3)CC1)C2=O ; Cc1cc(NCCN(C)C)cc2c1CN([C@H]1CC[C@@H](C(=O)Nc3ccn(CCO)n3)CC1)C2=O ; Cc1cc(NCCN(C)C)cc2c1CN([C@H]1CC[C@@H](C(=O)Nc3nc(CCO)cs3)CC1)C2=O]

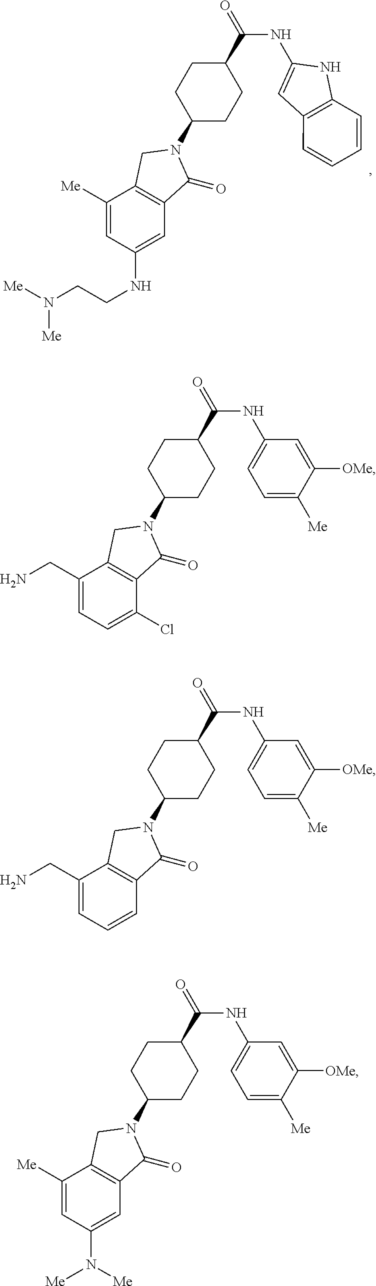 [SMILES: Cc1cc(NCCN(C)C)cc2c1CN([C@H]1CC[C@@H](C(=O)Nc3cc4ccccc4[nH]3)CC1)C2=O ; COc1cc(NC(=O)[C@H]2CC[C@@H](N3Cc4c(CN)ccc(Cl)c4C3=O)CC2)ccc1C ; COc1cc(NC(=O)[C@H]2CC[C@@H](N3Cc4c(CN)cccc4C3=O)CC2)ccc1C ; COc1cc(NC(=O)[C@H]2CC[C@@H](N3Cc4c(C)cc(N(C)C)cc4C3=O)CC2)ccc1C]

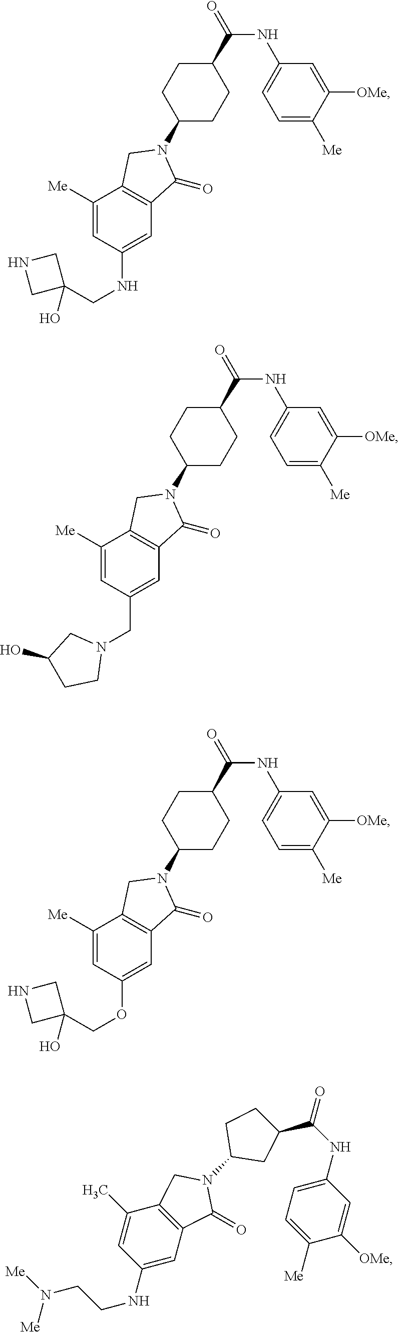 [SMILES: COc1cc(NC(=O)[C@H]2CC[C@@H](N3Cc4c(C)cc(NCC5(O)CNC5)cc4C3=O)CC2)ccc1C ; COc1cc(NC(=O)[C@H]2CC[C@@H](N3Cc4c(C)cc(CN5CC[C@@H](O)C5)cc4C3=O)CC2)ccc1C ; COc1cc(NC(=O)[C@H]2CC[C@@H](N3Cc4c(C)cc(OCC5(O)CNC5)cc4C3=O)CC2)ccc1C ; COc1cc(NC(=O)[C@@H]2CC[C@@H](N3Cc4c(C)cc(NCCN(C)C)cc4C3=O)C2)ccc1C]

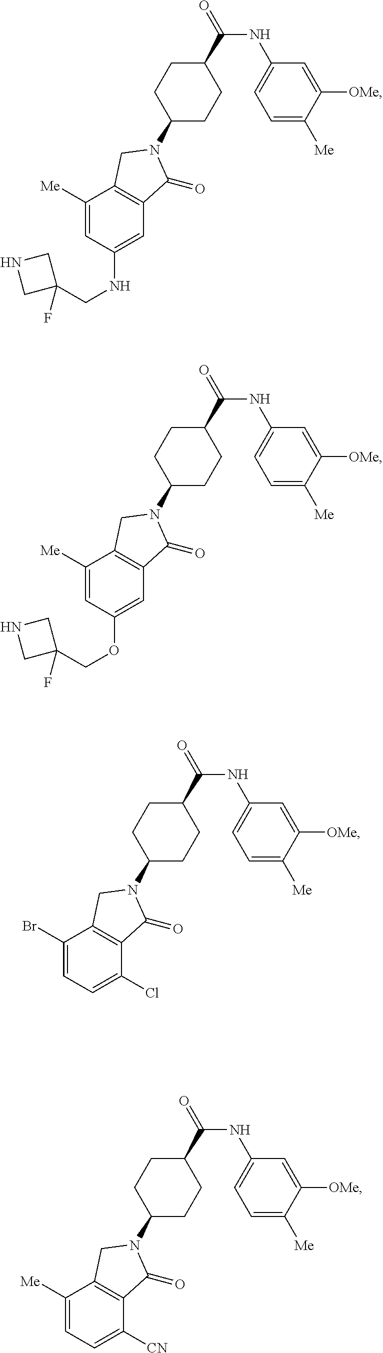 [SMILES: COc1cc(NC(=O)[C@H]2CC[C@@H](N3Cc4c(C)cc(NCC5(F)CNC5)cc4C3=O)CC2)ccc1C ; COc1cc(NC(=O)[C@H]2CC[C@@H](N3Cc4c(C)cc(OCC5(F)CNC5)cc4C3=O)CC2)ccc1C ; COc1cc(NC(=O)[C@H]2CC[C@@H](N3Cc4c(Br)ccc(Cl)c4C3=O)CC2)ccc1C ; COc1cc(NC(=O)[C@H]2CC[C@@H](N3Cc4c(C)ccc(C#N)c4C3=O)CC2)ccc1C]

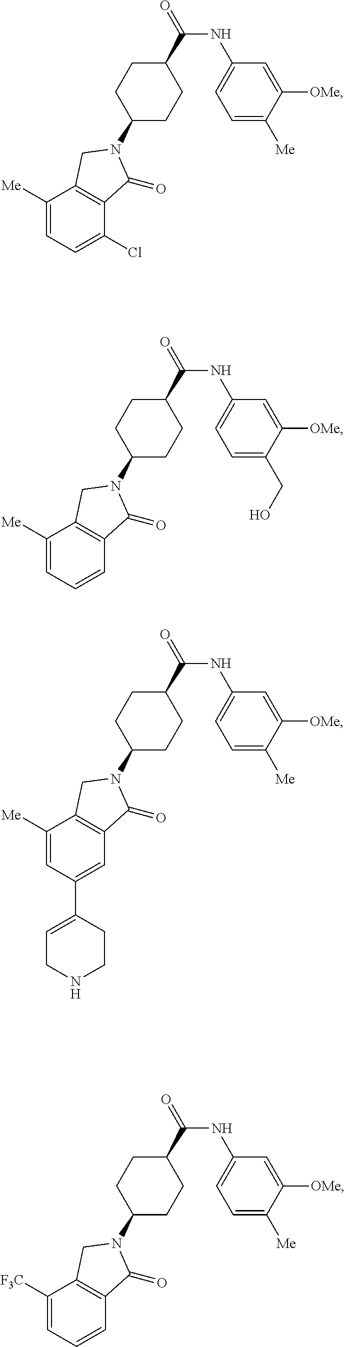 [SMILES: COc1cc(NC(=O)[C@H]2CC[C@@H](N3Cc4c(C)ccc(Cl)c4C3=O)CC2)ccc1C ; COc1cc(NC(=O)[C@H]2CC[C@@H](N3Cc4c(C)cccc4C3=O)CC2)ccc1CO ; COc1cc(NC(=O)[C@H]2CC[C@@H](N3Cc4c(C)cc(C5=CCNCC5)cc4C3=O)CC2)ccc1C ; COc1cc(NC(=O)[C@H]2CC[C@@H](N3Cc4c(cccc4C(F)(F)F)C3=O)CC2)ccc1C]

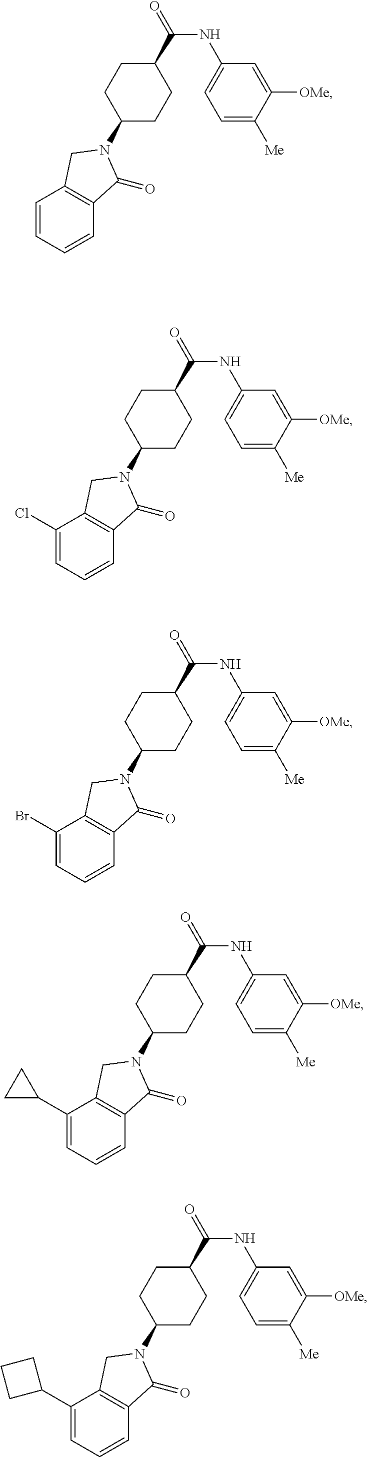 [SMILES: COc1cc(NC(=O)[C@H]2CC[C@@H](N3Cc4ccccc4C3=O)CC2)ccc1C ; COc1cc(NC(=O)[C@H]2CC[C@@H](N3Cc4c(Cl)cccc4C3=O)CC2)ccc1C ; COc1cc(NC(=O)[C@H]2CC[C@@H](N3Cc4c(Br)cccc4C3=O)CC2)ccc1C ; COc1cc(NC(=O)[C@H]2CC[C@@H](N3Cc4c(cccc4C4CC4)C3=O)CC2)ccc1C ; COc1cc(NC(=O)[C@H]2CC[C@@H](N3Cc4c(cccc4C4CCC4)C3=O)CC2)ccc1C]

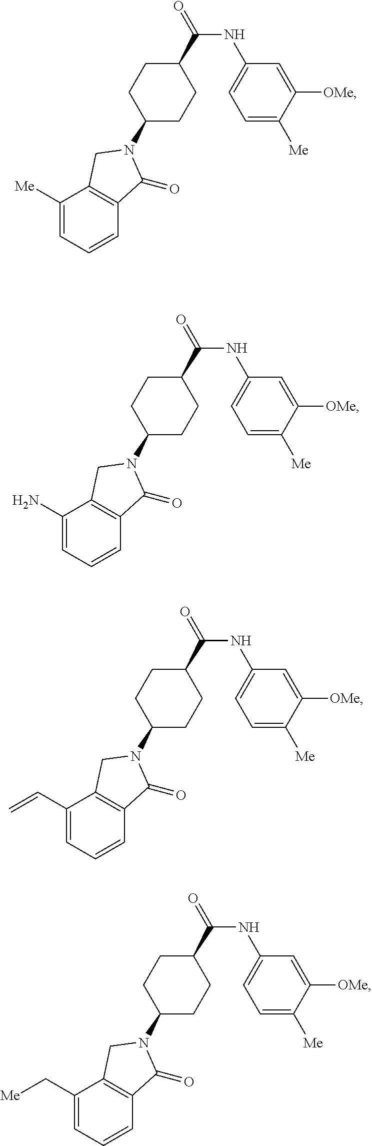 [SMILES: COc1cc(NC(=O)[C@H]2CC[C@@H](N3Cc4c(C)cccc4C3=O)CC2)ccc1C ; COc1cc(NC(=O)[C@H]2CC[C@@H](N3Cc4c(N)cccc4C3=O)CC2)ccc1C ; C=Cc1cccc2c1CN([C@H]1CC[C@@H](C(=O)Nc3ccc(C)c(OC)c3)CC1)C2=O ; CCc1cccc2c1CN([C@H]1CC[C@@H](C(=O)Nc3ccc(C)c(OC)c3)CC1)C2=O]

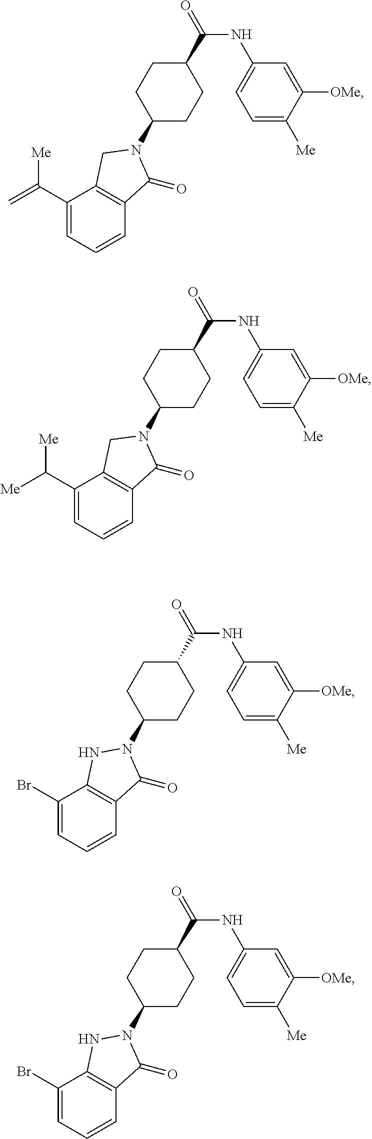 [SMILES: C=C(C)c1cccc2c1CN([C@H]1CC[C@@H](C(=O)Nc3ccc(C)c(OC)c3)CC1)C2=O ; COc1cc(NC(=O)[C@H]2CC[C@@H](N3Cc4c(cccc4C(C)C)C3=O)CC2)ccc1C ; COc1cc(NC(=O)[C@H]2CC[C@H](n3[nH]c4c(Br)cccc4c3=O)CC2)ccc1C ; COc1cc(NC(=O)[C@H]2CC[C@@H](n3[nH]c4c(Br)cccc4c3=O)CC2)ccc1C]

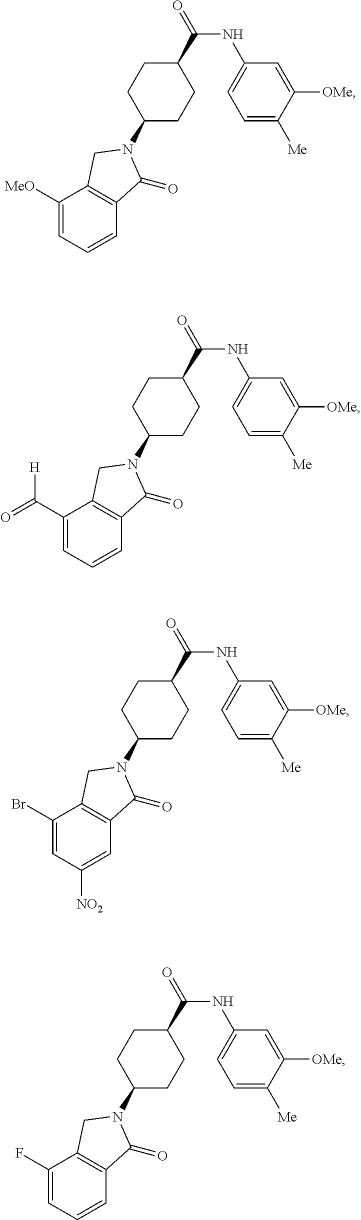 [SMILES: COc1cc(NC(=O)[C@H]2CC[C@@H](N3Cc4c(OC)cccc4C3=O)CC2)ccc1C ; COc1cc(NC(=O)[C@H]2CC[C@@H](N3Cc4c(C=O)cccc4C3=O)CC2)ccc1C ; COc1cc(NC(=O)[C@H]2CC[C@@H](N3Cc4c(Br)cc([N+](=O)[O-])cc4C3=O)CC2)ccc1C ; COc1cc(NC(=O)[C@H]2CC[C@@H](N3Cc4c(F)cccc4C3=O)CC2)ccc1C]

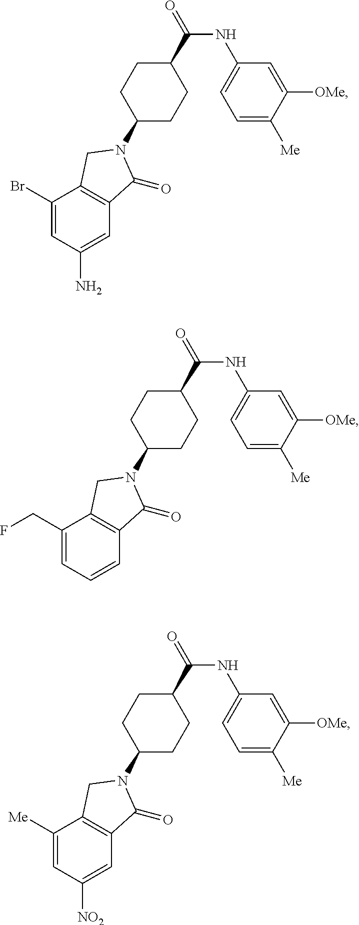 [SMILES: COc1cc(NC(=O)[C@H]2CC[C@@H](N3Cc4c(Br)cc(N)cc4C3=O)CC2)ccc1C ; COc1cc(NC(=O)[C@H]2CC[C@@H](N3Cc4c(CF)cccc4C3=O)CC2)ccc1C ; COc1cc(NC(=O)[C@H]2CC[C@@H](N3Cc4c(C)cc([N+](=O)[O-])cc4C3=O)CC2)ccc1C]

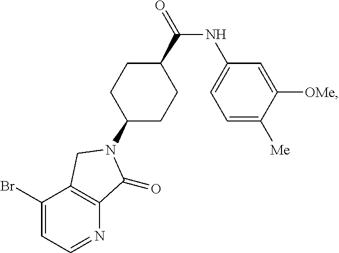 [SMILES: COc1cc(NC(=O)[C@H]2CC[C@@H](N3Cc4c(Br)ccnc4C3=O)CC2)ccc1C]

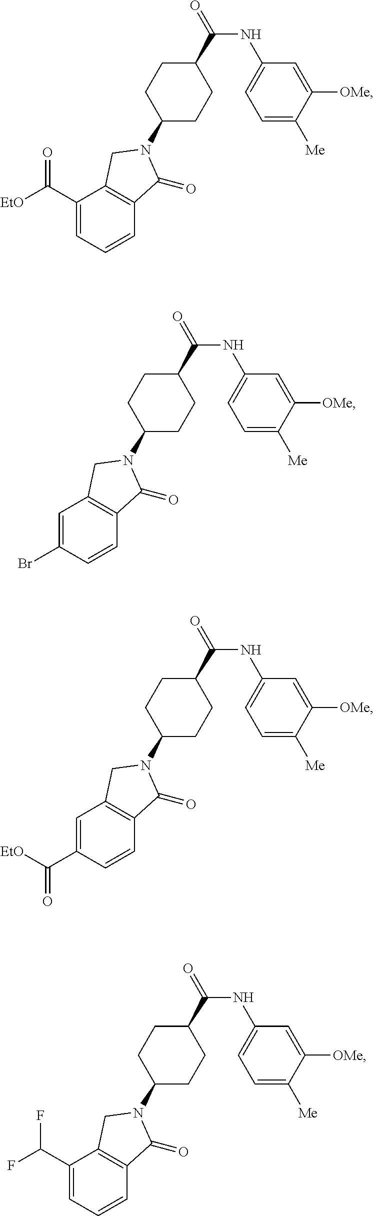 [SMILES: CCOC(=O)c1cccc2c1CN([C@H]1CC[C@@H](C(=O)Nc3ccc(C)c(OC)c3)CC1)C2=O ; COc1cc(NC(=O)[C@H]2CC[C@@H](N3Cc4cc(Br)ccc4C3=O)CC2)ccc1C ; CCOC(=O)c1ccc2c(c1)CN([C@H]1CC[C@@H](C(=O)Nc3ccc(C)c(OC)c3)CC1)C2=O ; COc1cc(NC(=O)[C@H]2CC[C@@H](N3Cc4c(cccc4C(F)F)C3=O)CC2)ccc1C]

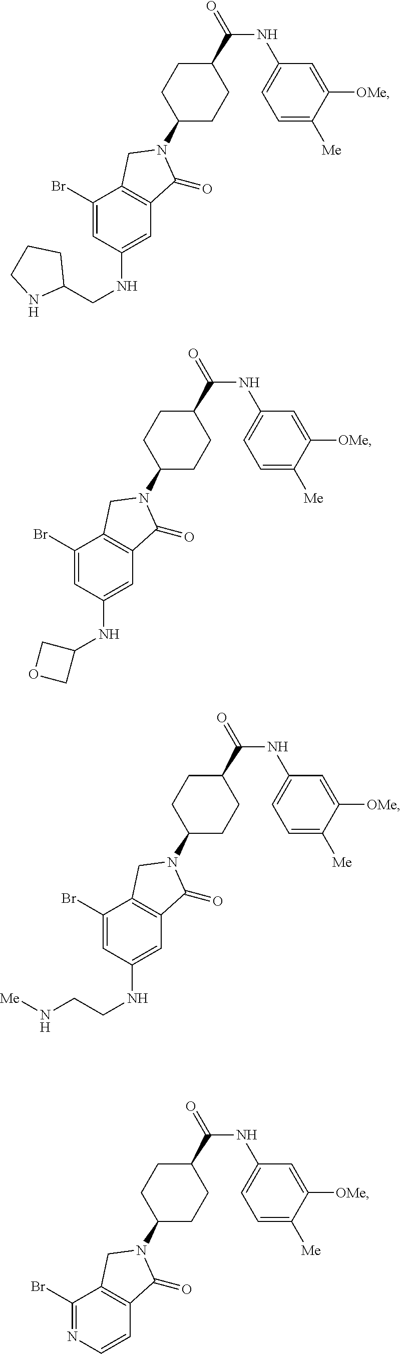 [SMILES: COc1cc(NC(=O)[C@H]2CC[C@@H](N3Cc4c(Br)cc(NCC5CCCN5)cc4C3=O)CC2)ccc1C ; COc1cc(NC(=O)[C@H]2CC[C@@H](N3Cc4c(Br)cc(NC5COC5)cc4C3=O)CC2)ccc1C ; CNCCNc1cc(Br)c2c(c1)C(=O)N([C@H]1CC[C@@H](C(=O)Nc3ccc(C)c(OC)c3)CC1)C2 ; COc1cc(NC(=O)[C@H]2CC[C@@H](N3Cc4c(ccnc4Br)C3=O)CC2)ccc1C]

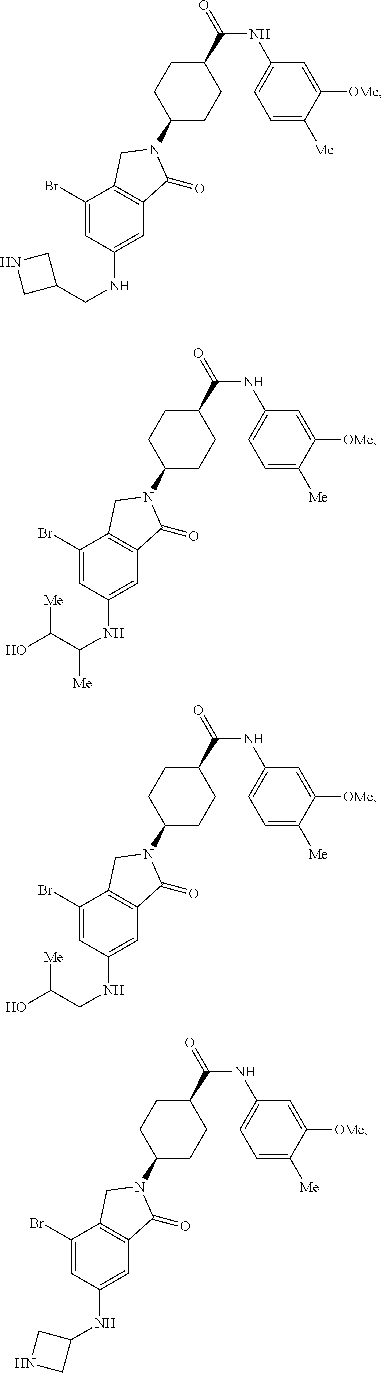 [SMILES: COc1cc(NC(=O)[C@H]2CC[C@@H](N3Cc4c(Br)cc(NCC5CNC5)cc4C3=O)CC2)ccc1C ; COc1cc(NC(=O)[C@H]2CC[C@@H](N3Cc4c(Br)cc(NC(C)C(C)O)cc4C3=O)CC2)ccc1C ; COc1cc(NC(=O)[C@H]2CC[C@@H](N3Cc4c(Br)cc(NCC(C)O)cc4C3=O)CC2)ccc1C ; COc1cc(NC(=O)[C@H]2CC[C@@H](N3Cc4c(Br)cc(NC5CNC5)cc4C3=O)CC2)ccc1C]

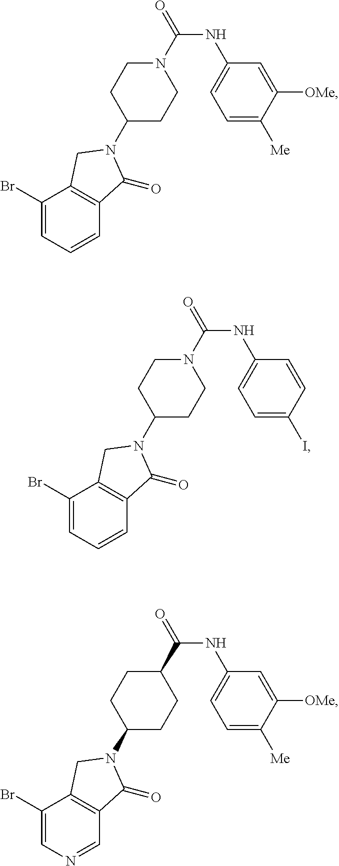 [SMILES: COc1cc(NC(=O)N2CCC(N3Cc4c(Br)cccc4C3=O)CC2)ccc1C ; O=C(Nc1ccc(I)cc1)N1CCC(N2Cc3c(Br)cccc3C2=O)CC1 ; COc1cc(NC(=O)[C@H]2CC[C@@H](N3Cc4c(Br)cncc4C3=O)CC2)ccc1C]

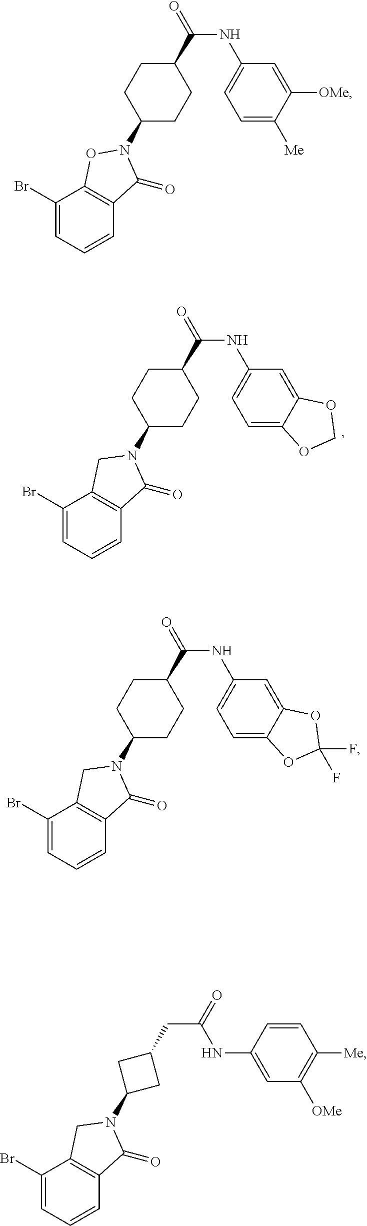 [SMILES: COc1cc(NC(=O)[C@H]2CC[C@@H](n3oc4c(Br)cccc4c3=O)CC2)ccc1C ; O=C1c2cccc(Br)c2CN1[C@H]1CC[C@@H](C(=O)Nc2ccc3c(c2)OCO3)CC1 ; O=C1c2cccc(Br)c2CN1[C@H]1CC[C@@H](C(=O)Nc2ccc3c(c2)OC(F)(F)O3)CC1 ; COc1cc(NC(=O)C[C@H]2C[C@H](N3Cc4c(Br)cccc4C3=O)C2)ccc1C]

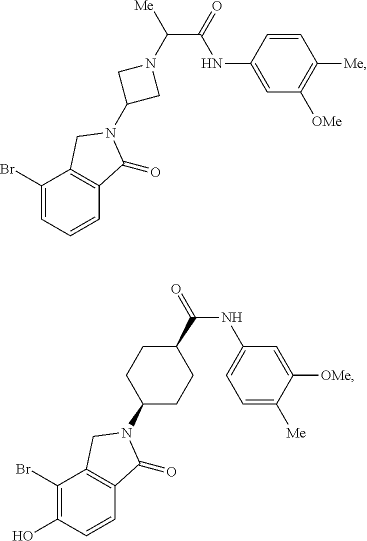 [SMILES: COc1cc(NC(=O)C(C)N2CC(N3Cc4c(Br)cccc4C3=O)C2)ccc1C ; COc1cc(NC(=O)[C@H]2CC[C@@H](N3Cc4c(ccc(O)c4Br)C3=O)CC2)ccc1C]

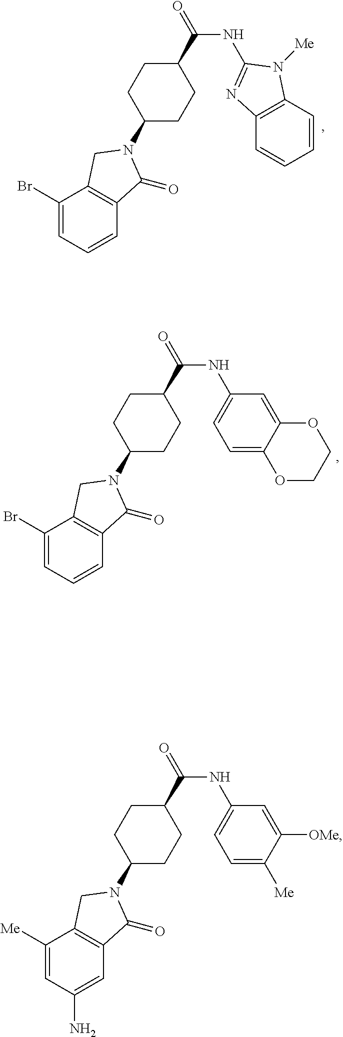 [SMILES: Cn1c(NC(=O)[C@H]2CC[C@@H](N3Cc4c(Br)cccc4C3=O)CC2)nc2ccccc21 ; O=C1c2cccc(Br)c2CN1[C@H]1CC[C@@H](C(=O)Nc2ccc3c(c2)OCCO3)CC1 ; COc1cc(NC(=O)[C@H]2CC[C@@H](N3Cc4c(C)cc(N)cc4C3=O)CC2)ccc1C]

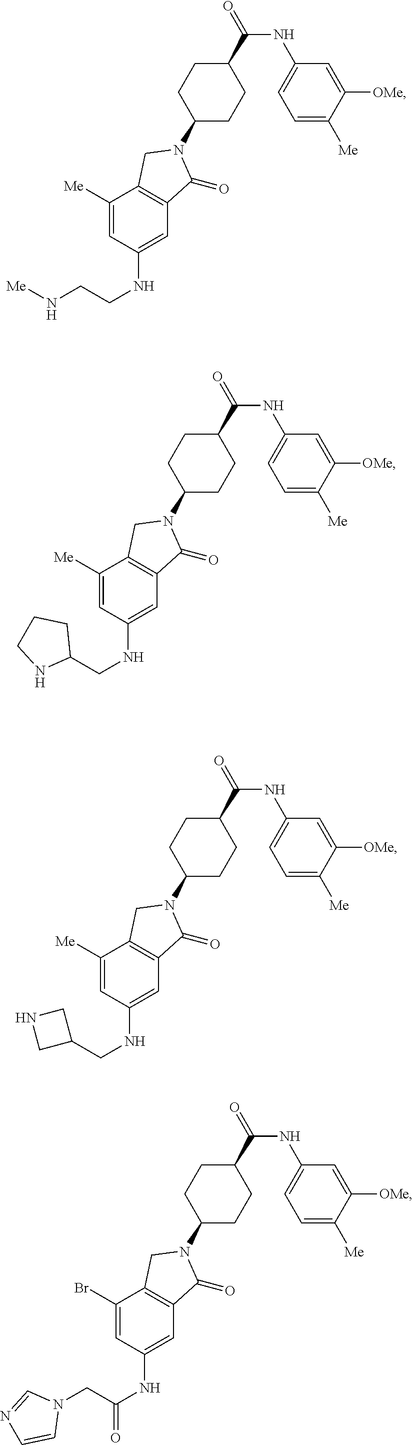 [SMILES: CNCCNc1cc(C)c2c(c1)C(=O)N([C@H]1CC[C@@H](C(=O)Nc3ccc(C)c(OC)c3)CC1)C2 ; COc1cc(NC(=O)[C@H]2CC[C@@H](N3Cc4c(C)cc(NCC5CCCN5)cc4C3=O)CC2)ccc1C ; COc1cc(NC(=O)[C@H]2CC[C@@H](N3Cc4c(C)cc(NCC5CNC5)cc4C3=O)CC2)ccc1C ; COc1cc(NC(=O)[C@H]2CC[C@@H](N3Cc4c(Br)cc(NC(=O)Cn5ccnc5)cc4C3=O)CC2)ccc1C]

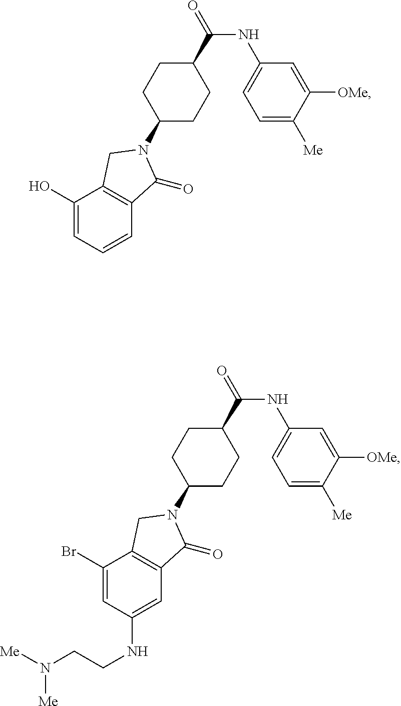 [SMILES: COc1cc(NC(=O)[C@H]2CC[C@@H](N3Cc4c(O)cccc4C3=O)CC2)ccc1C ; COc1cc(NC(=O)[C@H]2CC[C@@H](N3Cc4c(Br)cc(NCCN(C)C)cc4C3=O)CC2)ccc1C]

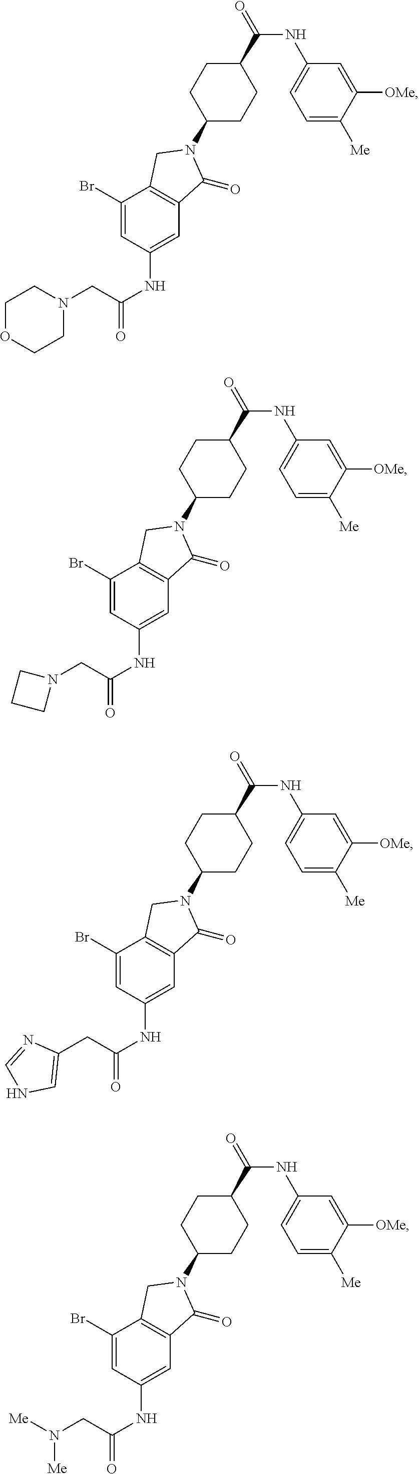 [SMILES: COc1cc(NC(=O)[C@H]2CC[C@@H](N3Cc4c(Br)cc(NC(=O)CN5CCOCC5)cc4C3=O)CC2)ccc1C ; COc1cc(NC(=O)[C@H]2CC[C@@H](N3Cc4c(Br)cc(NC(=O)CN5CCC5)cc4C3=O)CC2)ccc1C ; COc1cc(NC(=O)[C@H]2CC[C@@H](N3Cc4c(Br)cc(NC(=O)Cc5c[nH]cn5)cc4C3=O)CC2)ccc1C ; COc1cc(NC(=O)[C@H]2CC[C@@H](N3Cc4c(Br)cc(NC(=O)CN(C)C)cc4C3=O)CC2)ccc1C]

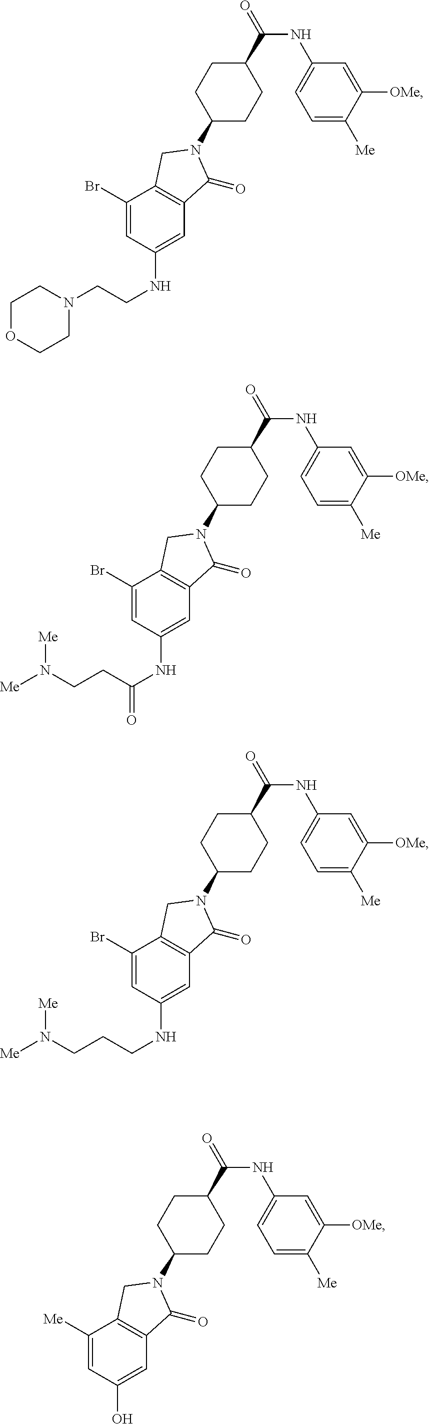 [SMILES: COc1cc(NC(=O)[C@H]2CC[C@@H](N3Cc4c(Br)cc(NCCN5CCOCC5)cc4C3=O)CC2)ccc1C ; COc1cc(NC(=O)[C@H]2CC[C@@H](N3Cc4c(Br)cc(NC(=O)CCN(C)C)cc4C3=O)CC2)ccc1C ; COc1cc(NC(=O)[C@H]2CC[C@@H](N3Cc4c(Br)cc(NCCCN(C)C)cc4C3=O)CC2)ccc1C ; COc1cc(NC(=O)[C@H]2CC[C@@H](N3Cc4c(C)cc(O)cc4C3=O)CC2)ccc1C]

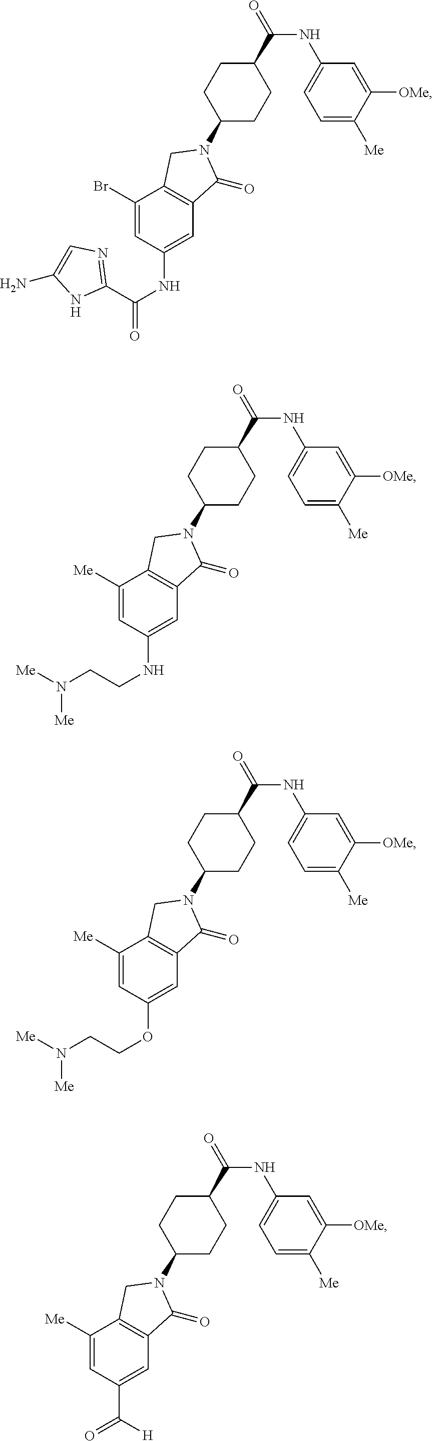 [SMILES: COc1cc(NC(=O)[C@H]2CC[C@@H](N3Cc4c(Br)cc(NC(=O)c5ncc(N)[nH]5)cc4C3=O)CC2)ccc1C ; COc1cc(NC(=O)[C@H]2CC[C@@H](N3Cc4c(C)cc(NCCN(C)C)cc4C3=O)CC2)ccc1C ; COc1cc(NC(=O)[C@H]2CC[C@@H](N3Cc4c(C)cc(OCCN(C)C)cc4C3=O)CC2)ccc1C ; COc1cc(NC(=O)[C@H]2CC[C@@H](N3Cc4c(C)cc(C=O)cc4C3=O)CC2)ccc1C]

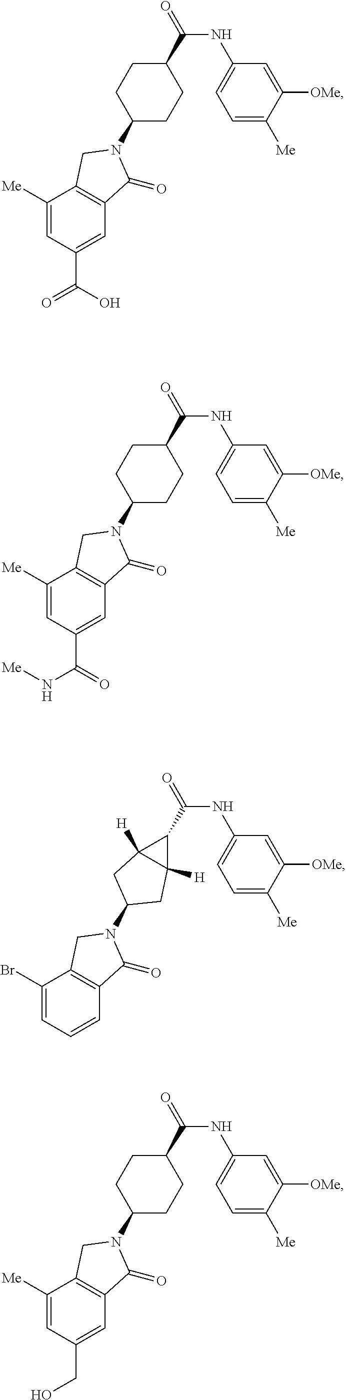 [SMILES: COc1cc(NC(=O)[C@H]2CC[C@@H](N3Cc4c(C)cc(C(=O)O)cc4C3=O)CC2)ccc1C ; CNC(=O)c1cc(C)c2c(c1)C(=O)N([C@H]1CC[C@@H](C(=O)Nc3ccc(C)c(OC)c3)CC1)C2 ; COc1cc(NC(=O)[C@@H]2[C@@H]3C[C@@H](N4Cc5c(Br)cccc5C4=O)C[C@@H]32)ccc1C ; COc1cc(NC(=O)[C@H]2CC[C@@H](N3Cc4c(C)cc(CO)cc4C3=O)CC2)ccc1C]

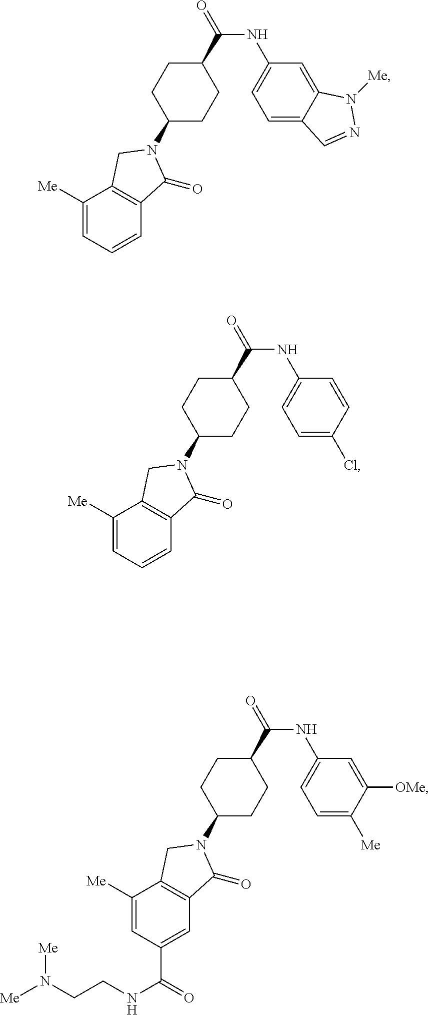 [SMILES: Cc1cccc2c1CN([C@H]1CC[C@@H](C(=O)Nc3ccc4cnn(C)c4c3)CC1)C2=O ; Cc1cccc2c1CN([C@H]1CC[C@@H](C(=O)Nc3ccc(Cl)cc3)CC1)C2=O ; COc1cc(NC(=O)[C@H]2CC[C@@H](N3Cc4c(C)cc(C(=O)NCCN(C)C)cc4C3=O)CC2)ccc1C]

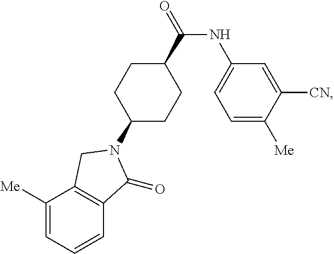 [SMILES: Cc1ccc(NC(=O)[C@H]2CC[C@@H](N3Cc4c(C)cccc4C3=O)CC2)cc1C#N]

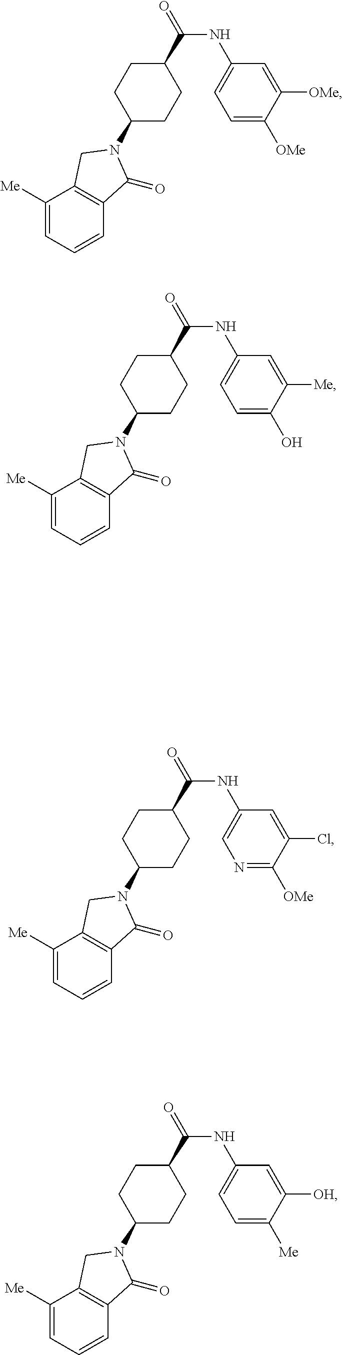 [SMILES: COc1ccc(NC(=O)[C@H]2CC[C@@H](N3Cc4c(C)cccc4C3=O)CC2)cc1OC ; Cc1cc(NC(=O)[C@H]2CC[C@@H](N3Cc4c(C)cccc4C3=O)CC2)ccc1O ; COc1ncc(NC(=O)[C@H]2CC[C@@H](N3Cc4c(C)cccc4C3=O)CC2)cc1Cl ; Cc1ccc(NC(=O)[C@H]2CC[C@@H](N3Cc4c(C)cccc4C3=O)CC2)cc1O]

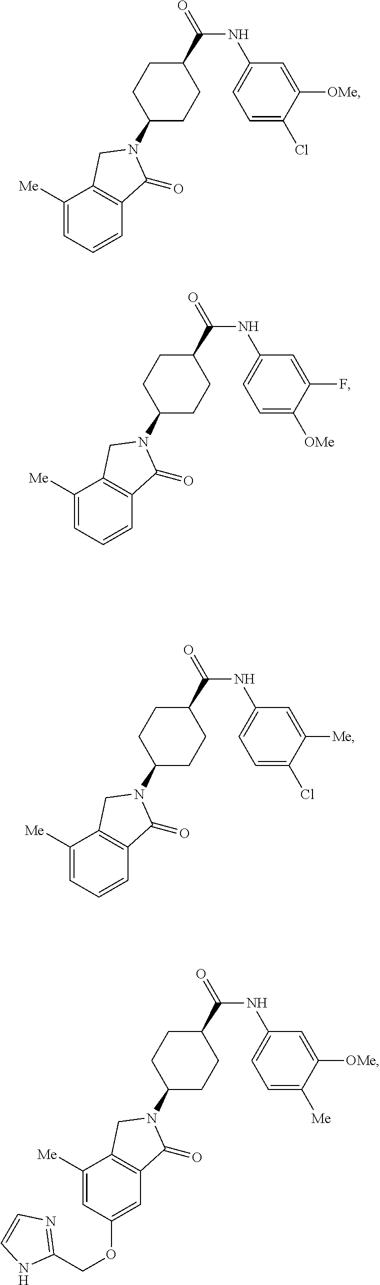 [SMILES: COc1cc(NC(=O)[C@H]2CC[C@@H](N3Cc4c(C)cccc4C3=O)CC2)ccc1Cl ; COc1ccc(NC(=O)[C@H]2CC[C@@H](N3Cc4c(C)cccc4C3=O)CC2)cc1F ; Cc1cc(NC(=O)[C@H]2CC[C@@H](N3Cc4c(C)cccc4C3=O)CC2)ccc1Cl ; COc1cc(NC(=O)[C@H]2CC[C@@H](N3Cc4c(C)cc(OCc5ncc[nH]5)cc4C3=O)CC2)ccc1C]

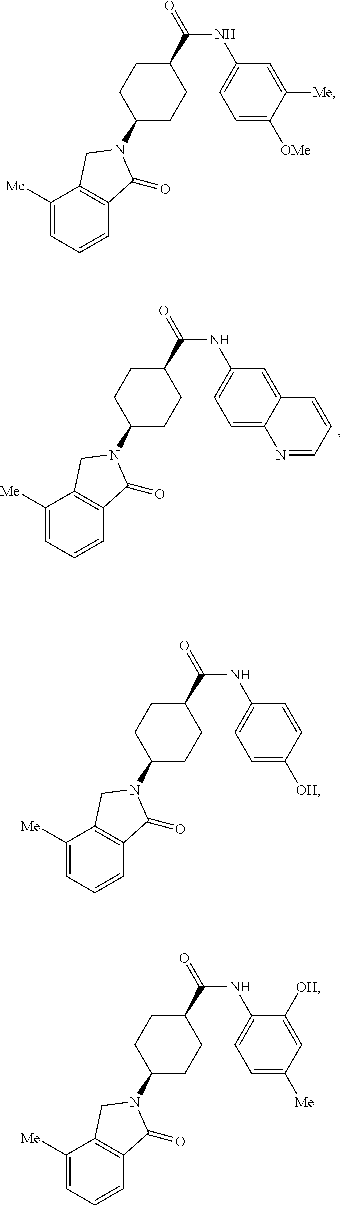 [SMILES: COc1ccc(NC(=O)[C@H]2CC[C@@H](N3Cc4c(C)cccc4C3=O)CC2)cc1C ; Cc1cccc2c1CN([C@H]1CC[C@@H](C(=O)Nc3ccc4ncccc4c3)CC1)C2=O ; Cc1cccc2c1CN([C@H]1CC[C@@H](C(=O)Nc3ccc(O)cc3)CC1)C2=O ; Cc1ccc(NC(=O)[C@H]2CC[C@@H](N3Cc4c(C)cccc4C3=O)CC2)c(O)c1]

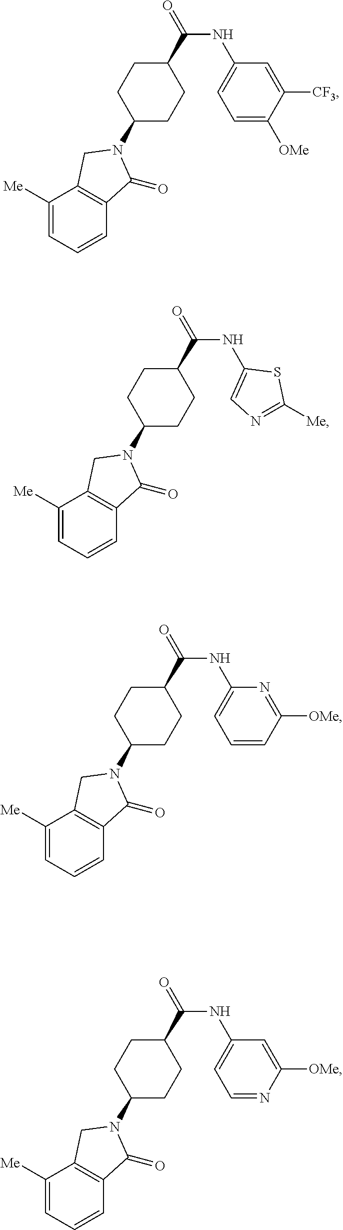 [SMILES: COc1ccc(NC(=O)[C@H]2CC[C@@H](N3Cc4c(C)cccc4C3=O)CC2)cc1C(F)(F)F ; Cc1ncc(NC(=O)[C@H]2CC[C@@H](N3Cc4c(C)cccc4C3=O)CC2)s1 ; COc1cccc(NC(=O)[C@H]2CC[C@@H](N3Cc4c(C)cccc4C3=O)CC2)n1 ; COc1cc(NC(=O)[C@H]2CC[C@@H](N3Cc4c(C)cccc4C3=O)CC2)ccn1]

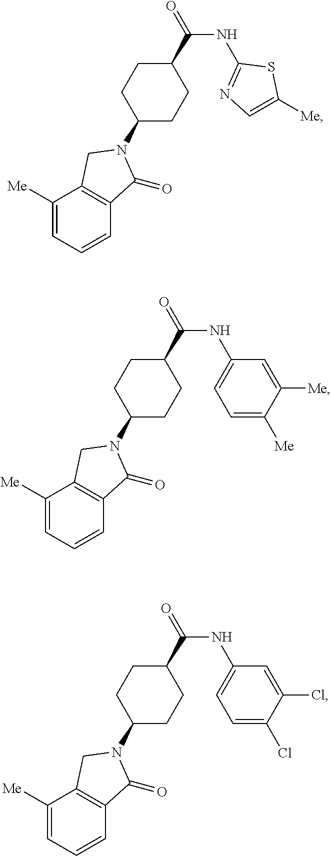 [SMILES: Cc1cnc(NC(=O)[C@H]2CC[C@@H](N3Cc4c(C)cccc4C3=O)CC2)s1 ; Cc1ccc(NC(=O)[C@H]2CC[C@@H](N3Cc4c(C)cccc4C3=O)CC2)cc1C ; Cc1cccc2c1CN([C@H]1CC[C@@H](C(=O)Nc3ccc(Cl)c(Cl)c3)CC1)C2=O]

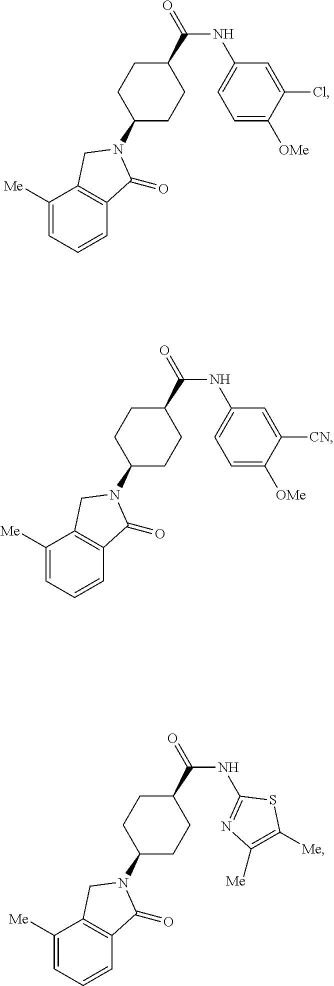 [SMILES: COc1ccc(NC(=O)[C@H]2CC[C@@H](N3Cc4c(C)cccc4C3=O)CC2)cc1Cl ; COc1ccc(NC(=O)[C@H]2CC[C@@H](N3Cc4c(C)cccc4C3=O)CC2)cc1C#N ; Cc1cccc2c1CN([C@H]1CC[C@@H](C(=O)Nc3nc(C)c(C)s3)CC1)C2=O]

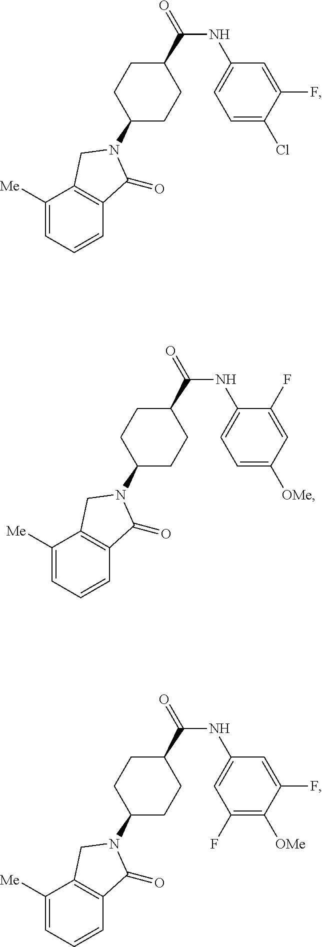 [SMILES: Cc1cccc2c1CN([C@H]1CC[C@@H](C(=O)Nc3ccc(Cl)c(F)c3)CC1)C2=O ; COc1ccc(NC(=O)[C@H]2CC[C@@H](N3Cc4c(C)cccc4C3=O)CC2)c(F)c1 ; COc1c(F)cc(NC(=O)[C@H]2CC[C@@H](N3Cc4c(C)cccc4C3=O)CC2)cc1F]

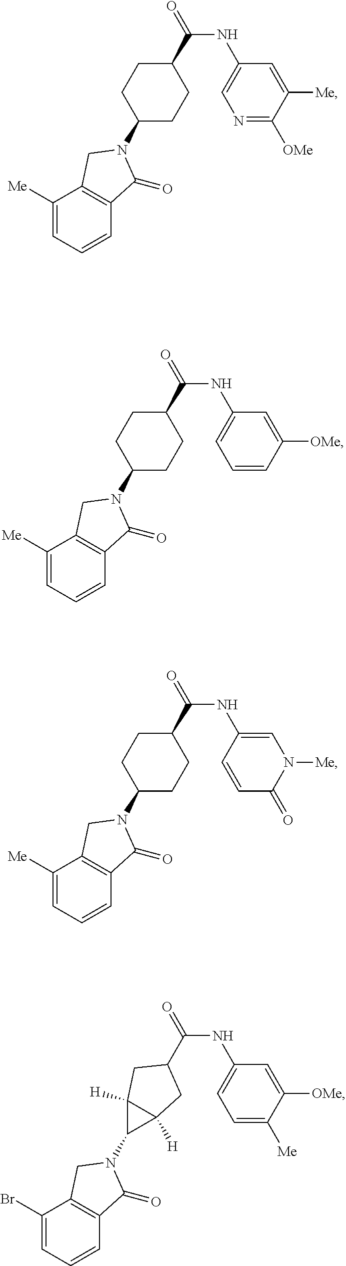 [SMILES: COc1ncc(NC(=O)[C@H]2CC[C@@H](N3Cc4c(C)cccc4C3=O)CC2)cc1C ; COc1cccc(NC(=O)[C@H]2CC[C@@H](N3Cc4c(C)cccc4C3=O)CC2)c1 ; Cc1cccc2c1CN([C@H]1CC[C@@H](C(=O)Nc3ccc(=O)n(C)c3)CC1)C2=O ; COc1cc(NC(=O)C2C[C@@H]3[C@H](C2)[C@H]3N2Cc3c(Br)cccc3C2=O)ccc1C]

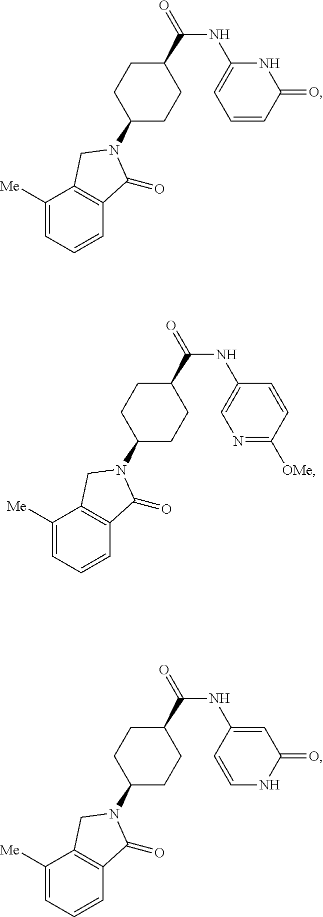 [SMILES: Cc1cccc2c1CN([C@H]1CC[C@@H](C(=O)Nc3cccc(=O)[nH]3)CC1)C2=O ; COc1ccc(NC(=O)[C@H]2CC[C@@H](N3Cc4c(C)cccc4C3=O)CC2)cn1 ; Cc1cccc2c1CN([C@H]1CC[C@@H](C(=O)Nc3cc[nH]c(=O)c3)CC1)C2=O]

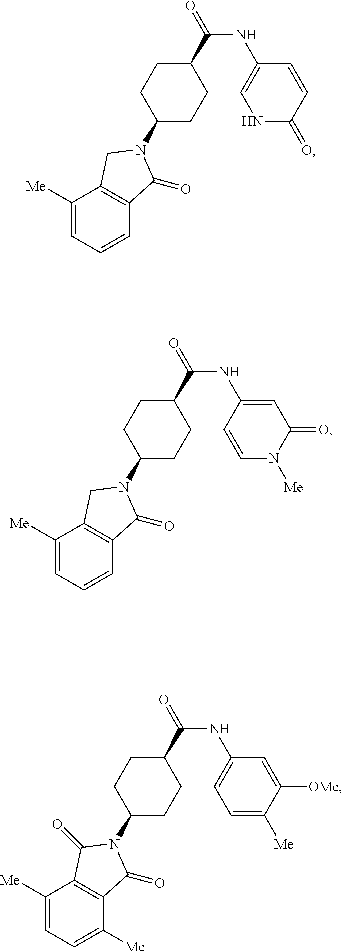 [SMILES: Cc1cccc2c1CN([C@H]1CC[C@@H](C(=O)Nc3ccc(=O)[nH]c3)CC1)C2=O ; Cc1cccc2c1CN([C@H]1CC[C@@H](C(=O)Nc3ccn(C)c(=O)c3)CC1)C2=O ; COc1cc(NC(=O)[C@H]2CC[C@@H](N3C(=O)c4c(C)ccc(C)c4C3=O)CC2)ccc1C]

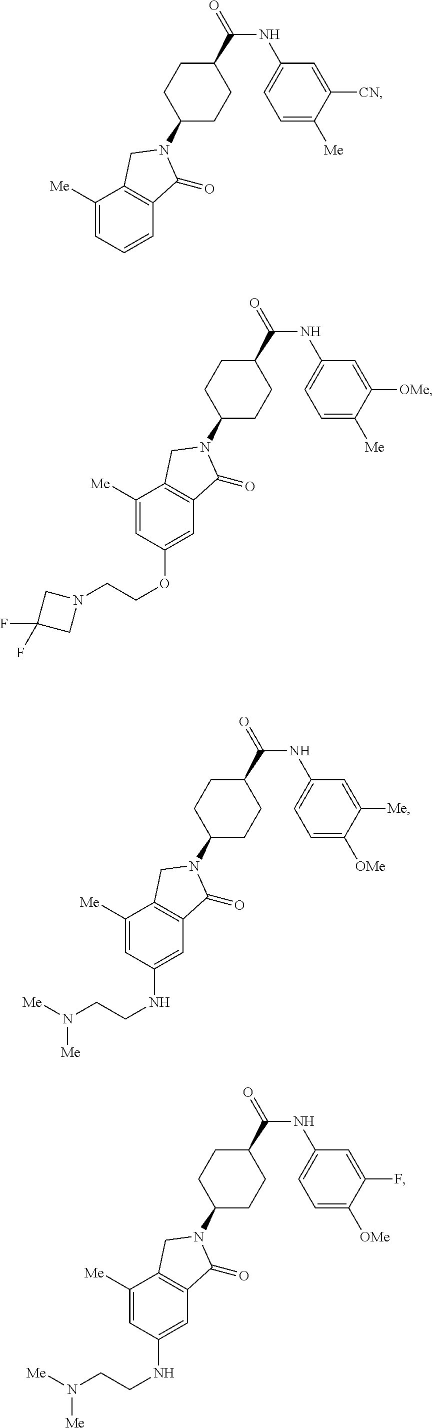 [SMILES: Cc1ccc(NC(=O)[C@H]2CC[C@@H](N3Cc4c(C)cccc4C3=O)CC2)cc1C#N ; COc1cc(NC(=O)[C@H]2CC[C@@H](N3Cc4c(C)cc(OCCN5CC(F)(F)C5)cc4C3=O)CC2)ccc1C ; COc1ccc(NC(=O)[C@H]2CC[C@@H](N3Cc4c(C)cc(NCCN(C)C)cc4C3=O)CC2)cc1C ; COc1ccc(NC(=O)[C@H]2CC[C@@H](N3Cc4c(C)cc(NCCN(C)C)cc4C3=O)CC2)cc1F]

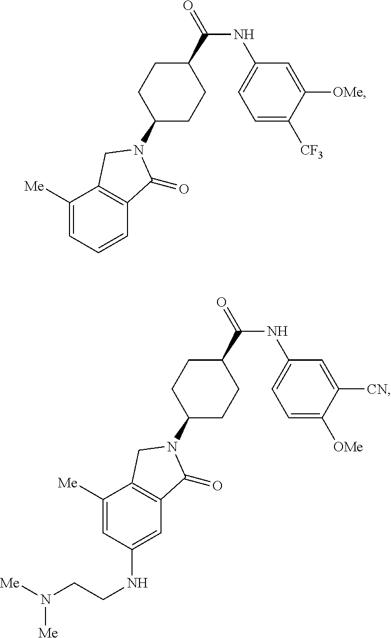 [SMILES: COc1cc(NC(=O)[C@H]2CC[C@@H](N3Cc4c(C)cccc4C3=O)CC2)ccc1C(F)(F)F ; COc1ccc(NC(=O)[C@H]2CC[C@@H](N3Cc4c(C)cc(NCCN(C)C)cc4C3=O)CC2)cc1C#N]

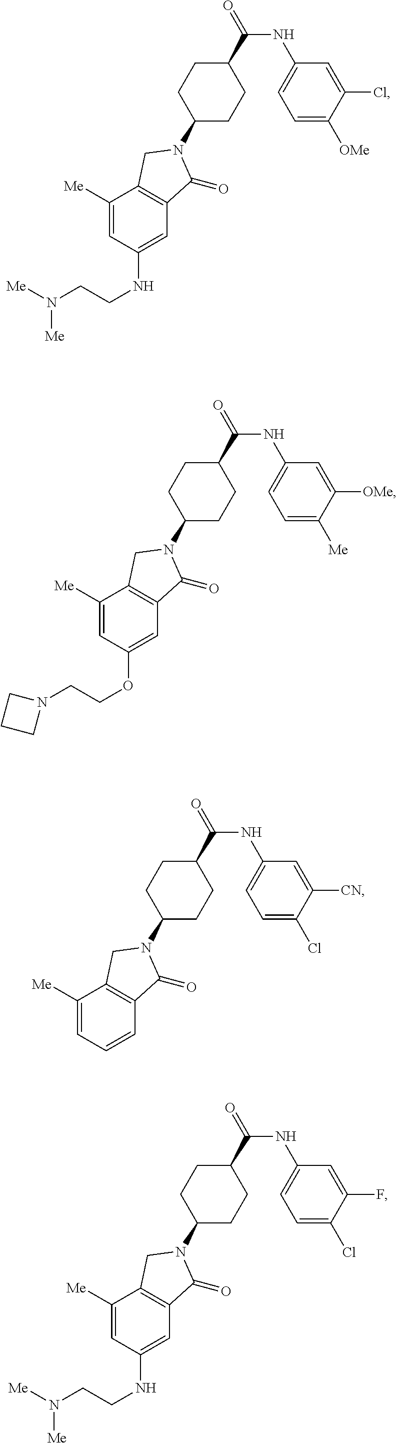 [SMILES: COc1ccc(NC(=O)[C@H]2CC[C@@H](N3Cc4c(C)cc(NCCN(C)C)cc4C3=O)CC2)cc1Cl ; COc1cc(NC(=O)[C@H]2CC[C@@H](N3Cc4c(C)cc(OCCN5CCC5)cc4C3=O)CC2)ccc1C ; Cc1cccc2c1CN([C@H]1CC[C@@H](C(=O)Nc3ccc(Cl)c(C#N)c3)CC1)C2=O ; Cc1cc(NCCN(C)C)cc2c1CN([C@H]1CC[C@@H](C(=O)Nc3ccc(Cl)c(F)c3)CC1)C2=O]

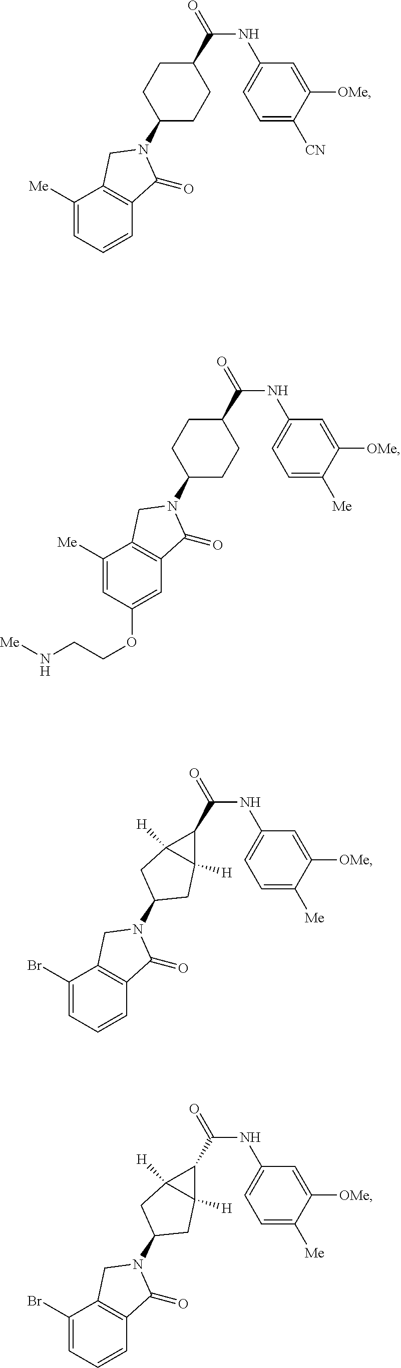 [SMILES: COc1cc(NC(=O)[C@H]2CC[C@@H](N3Cc4c(C)cccc4C3=O)CC2)ccc1C#N ; CNCCOc1cc(C)c2c(c1)C(=O)N([C@H]1CC[C@@H](C(=O)Nc3ccc(C)c(OC)c3)CC1)C2 ; COc1cc(NC(=O)[C@@H]2[C@@H]3C[C@H](N4Cc5c(Br)cccc5C4=O)C[C@@H]32)ccc1C ; COc1cc(NC(=O)[C@H]2[C@@H]3C[C@H](N4Cc5c(Br)cccc5C4=O)C[C@@H]32)ccc1C]

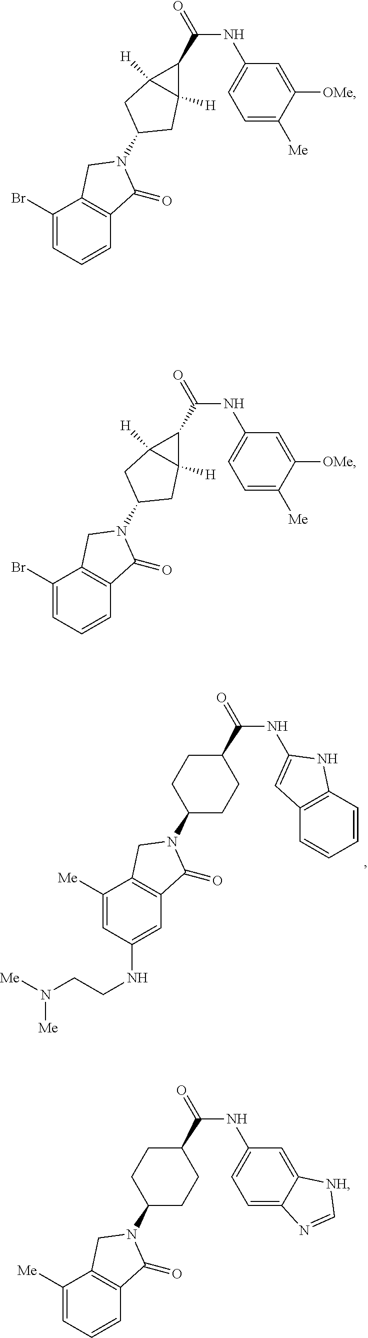 [SMILES: COc1cc(NC(=O)[C@@H]2[C@@H]3C[C@@H](N4Cc5c(Br)cccc5C4=O)C[C@@H]32)ccc1C ; COc1cc(NC(=O)[C@H]2[C@@H]3C[C@@H](N4Cc5c(Br)cccc5C4=O)C[C@@H]32)ccc1C ; Cc1cc(NCCN(C)C)cc2c1CN([C@H]1CC[C@@H](C(=O)Nc3cc4ccccc4[nH]3)CC1)C2=O ; Cc1cccc2c1CN([C@H]1CC[C@@H](C(=O)Nc3ccc4nc[nH]c4c3)CC1)C2=O]

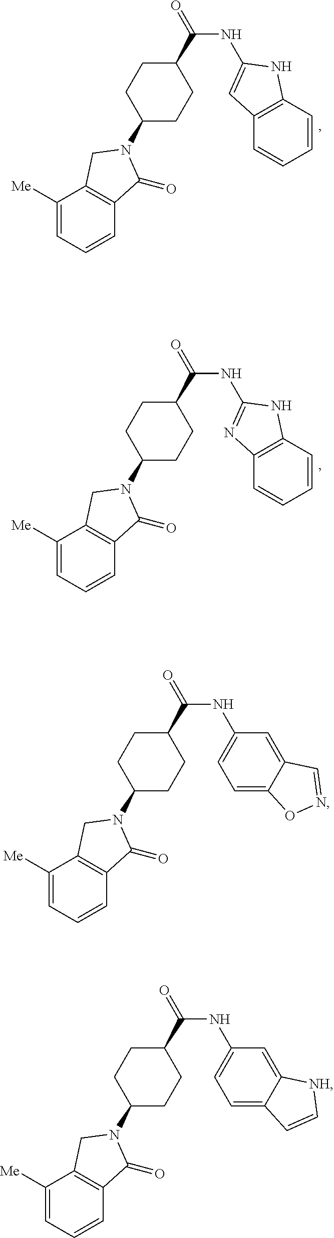 [SMILES: Cc1cccc2c1CN([C@H]1CC[C@@H](C(=O)Nc3cc4ccccc4[nH]3)CC1)C2=O ; Cc1cccc2c1CN([C@H]1CC[C@@H](C(=O)Nc3nc4ccccc4[nH]3)CC1)C2=O ; Cc1cccc2c1CN([C@H]1CC[C@@H](C(=O)Nc3ccc4oncc4c3)CC1)C2=O ; Cc1cccc2c1CN([C@H]1CC[C@@H](C(=O)Nc3ccc4cc[nH]c4c3)CC1)C2=O]

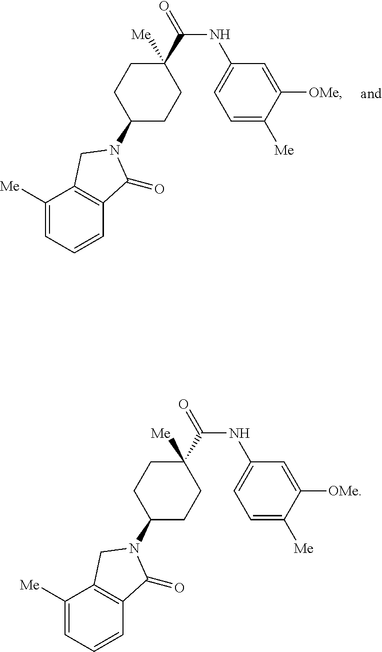 [SMILES: COc1cc(NC(=O)[C@]2(C)CC[C@H](N3Cc4c(C)cccc4C3=O)CC2)ccc1C ; COc1cc(NC(=O)[C@]2(C)CC[C@@H](N3Cc4c(C)cccc4C3=O)CC2)ccc1C]

23. The compound of claim 1, or a stereoisomer, or a pharmaceutically acceptable salt thereof, selected from the group consisting of

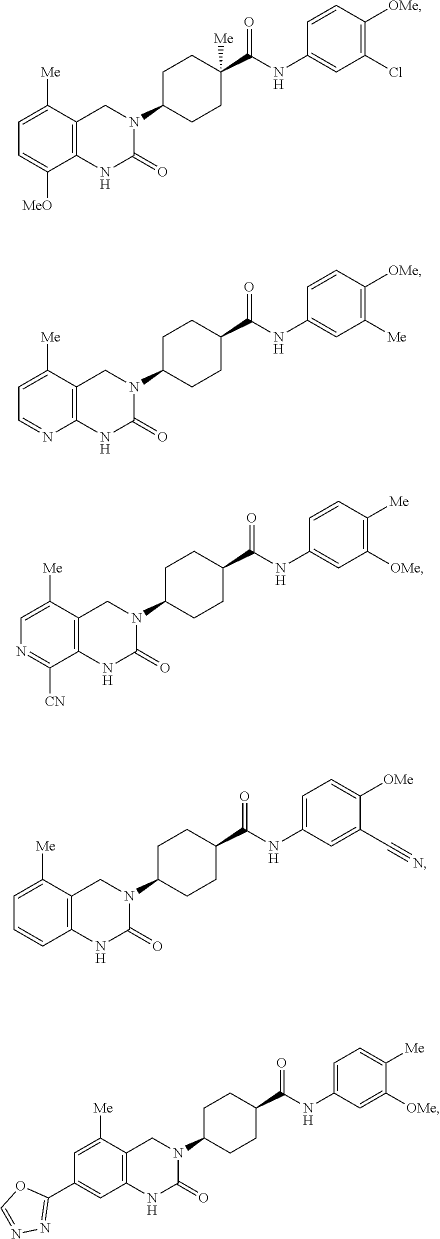 [SMILES: COc1ccc(NC(=O)[C@]2(C)CC[C@H](N3Cc4c(C)ccc(OC)c4NC3=O)CC2)cc1Cl ; COc1ccc(NC(=O)[C@H]2CC[C@@H](N3Cc4c(C)ccnc4NC3=O)CC2)cc1C ; COc1cc(NC(=O)[C@H]2CC[C@@H](N3Cc4c(C)cnc(C#N)c4NC3=O)CC2)ccc1C ; COc1ccc(NC(=O)[C@H]2CC[C@@H](N3Cc4c(C)cccc4NC3=O)CC2)cc1C#N ; COc1cc(NC(=O)[C@H]2CC[C@@H](N3Cc4c(C)cc(-c5nnco5)cc4NC3=O)CC2)ccc1C]

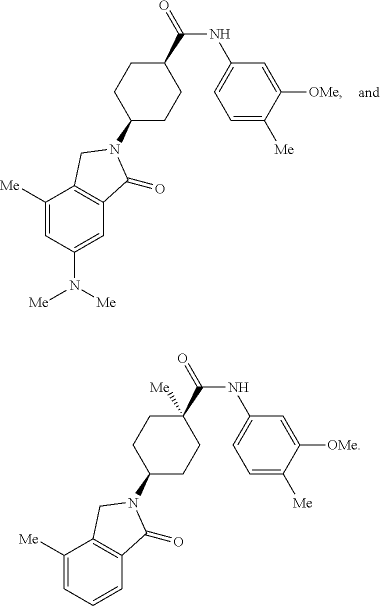 [SMILES: COc1cc(NC(=O)[C@H]2CC[C@@H](N3Cc4c(C)cc(N(C)C)cc4C3=O)CC2)ccc1C ; COc1cc(NC(=O)[C@]2(C)CC[C@H](N3Cc4c(C)cccc4C3=O)CC2)ccc1C]

24-32. (canceled)

33. A method of treating a disorder in a subject, comprising: administering to the subject a compound of claim 1, or a stereoisomer or a pharmaceutically acceptable salt thereof;
wherein the disorder is mediated by OGG1.

34. The method of claim 33, wherein the disorder is selected from the group consisting of cancer, cardiovascular disorder, neurological disorder, inflammatory disorder, autoimmune disorder, and infectious disease.

35. The method of claim 33, wherein the disorder is cancer, and the cancer is of solid organ origin or of hematopoietic origin, wherein the solid organ is selected from the group consisting of the brain, breast, colon, endometrium, esophagus, head and neck, upper gastrointestinal tract, respiratory tract, lung, kidney, liver, lower gastrointestinal tract, small intestine, large intestine, ovary, pancreas, prostate, stomach, testes, and urinary tract; or
the cancer is leukemia or lymphoma, wherein the leukemia is acute lymphocytic leukemia (ALL), acute myelogenous leukemia (AML), chronic lymphocytic leukemia (CLL), or chronic myelogenous leukemia (CML), and the lymphoma is Hodgkin's lymphoma, non-Hodgkin's lymphoma, multiple myeloma, B-cell lymphoma, diffuse large B-cell lymphoma (DLBCL), chronic lymphocytic lymphoma (CLL), T-cell lymphoma, hairy cell lymphoma, or Burkett's lymphoma; or
the cancer is selected from the group consisting of cancer of the bladder, bone cancer, cancer of the cervix, cancer of the epithelium, cancer of the gallbladder, cancer of the rectum, skin cancer, thyroid cancer, and cancer of the uterus.

36-40. (canceled)

41. The method of claim 33, wherein the disorder is the inflammatory disorder, and the inflammatory disorder is selected from the group consisting of inflammatory bowel disease (IBD), atherosclerosis, rheumatoid arthritis, fibrosis, Alzheimer's disease, and Parkinson's disease.
